(19) United States
(12) Patent Application Publication
(10) Pub. No.: US 2026/0070917 A1
(43) Pub. Date: Mar. 12, 2026

(54) CYCLIN DEPENDENT KINASE DEGRADERS AND METHODS OF USE THEREOF

(71) Applicant: Differentiated Therapeutics Inc, Oceanside, CA (US)

(72) Inventors: Timothy A. DWIGHT, Quincy, MA (US); Bryce K. ALLEN, Miami, FL (US)

(21) Appl. No.: 19/298,091

(22) Filed: Aug. 12, 2025

Related U.S. Application Data
(63) Continuation of application No. PCT/US2024/015618, filed on Feb. 13, 2024.
(60) Provisional application No. 63/445,272, filed on Feb. 13, 2023.
(60) Provisional application No. 63/445,271, filed on Feb. 13, 2023.
(60) Provisional application No. 63/509,837, filed on Jun. 23, 2023.

(51) Int. Cl. C07D 487/10; A61K 31/527; A61K 31/551; C07D 519/00

(52) U.S. Cl. CPC C07D 487/10; A61K 31/527; A61K 31/551; C07D 519/00

(57) ABSTRACT

The present disclosure relates to novel compounds and pharmaceutical compositions thereof, and methods for degrading CDK2 and/or CCNE1 with the compounds and compositions of the disclosure. The present disclosure further relates to, but is not limited to, methods for treating disorders associated with CDK2 and/or CCNE1 with the compounds and compositions of the disclosure.

### CROSS-REFERENCE TO RELATED APPLICATIONS

This application claims the benefit of PCT/US2024/015618 filed on Feb. 13, 2024, which claims the benefit of U.S. Provisional Patent Application No. 63/445,271, filed Feb. 13, 2023, U.S. Provisional Patent Application No. 63/445,272, filed Feb. 13, 2023, and U.S. Provisional Patent Application No. 63/509,837, filed Jun. 23, 2023, the entire contents of which are incorporated by reference herein.

### TECHNICAL FIELD OF THE INVENTION

The present invention relates to compounds and methods useful for the modulation of cyclin dependent kinase 2 (CDK2) and/or cyclin E (CCNE1 and/or CCNE2) via ubiquitination and/or degradation by compounds according to the present invention. The invention also provides pharmaceutically acceptable compositions comprising compounds of the present invention and methods of using said compositions in the treatment of various disorders.

### BACKGROUND OF THE INVENTION

Cyclin-dependent kinases (CDKs) are a family of serine/threonine kinases. Heterodimerized with regulatory subunits known as cyclins, CDKs become fully activated and regulate key cellular processes including cell cycle progression and cell division (Morgan, D. O., Annu Rev Cell Dev Biol, 1997. 13: 261-91). Uncontrolled proliferation is a hallmark of cancer cells. The deregulation of the CDK activity is associated with abnormal regulation of cell-cycle, and is detected in virtually all forms of human cancers (Sherr, C. J., Science, 1996. 274(5293): 1672-7).

CDK2 is of particular interest because deregulation of CDK2 activity occurs frequently in a variety of human cancers. CDK2 plays a crucial role in promoting G1/S transition and S phase progression. In complex with cyclin E (CCNE), CDK2 phosphorylates retinoblastoma pocket protein family members (p107, p130, pRb), leading to de-repression of E2F transcription factors, expression of G1/S transition related genes and transition from G1 to S phase (Henley, S. A. and F. A. Dick, Cell Div, 2012, 7(1): p. 10). This in turn enables activation of CDK2/cyclin A, which phosphorylates endogenous substrates that permit DNA synthesis, replication and centrosome duplication (Ekholm, S. V. and S. I. Reed, Curr Opin Cell Biol, 2000. 12(6): 676-84). It has been reported that the CDK2 pathway influences tumorigenesis mainly through amplification and/or overexpression of CCNE1 and mutations that inactivate CDK2 endogenous inhibitors (e.g., p27), respectively (Xu, X., et al., Biochemistry, 1999. 38(27): 8713-22).

CCNE1 copy-number gain and overexpression have been identified in ovarian, gastric, endometrial, breast and other cancers and been associated with poor outcomes in these tumors (Keyomarsi, K., et al., N Engl J Med, 2002. 347(20): 1566-75; Nakayama, N., et al., Cancer, 2010. 116(11): 2621-34; Au-Yeung, G., et al., Clin Cancer Res, 2017. 23(7): 1862-1874; Rosen, D. G., et al., Cancer, 2006. 106(9): 1925-32). Amplification and/or overexpression of CCNE1 also reportedly contribute to trastuzumab resistance in HER2+ breast cancer and resistance to CDK4/6 inhibitors in estrogen receptor-positive breast cancer (Scaltriti, M., et al., Proc Natl Acad Sci USA, 2011. 108(9): 3761-6; Herrera-Abreu, M. T., et al., Cancer Res, 2016. 76(8): 2301-13). Various approaches targeting CDK2 have been shown to induce cell cycle arrest and tumor growth inhibition (Chen, Y N., et al., Proc Natl Acad Sci USA, 1999. 96(8): 4325-9; Mendoza, N., et al., Cancer Res, 2003. 63(5): 1020-4). Inhibition of CDK2 also reportedly restores sensitivity to trastuzumab treatment in resistant HER2+ breast tumors in a preclinical model (Scaltriti, supra).

Ubiquitin-Proteasome Pathway (UPP) is a critical pathway that regulates key regulator proteins and degrades misfolded or abnormal proteins. UPP is central to multiple cellular processes, and if defective or imbalanced, it leads to pathogenesis of a variety of diseases. The covalent attachment of ubiquitin to specific protein substrates is achieved through the action of E3 ubiquitin ligases.

There are over 600 E3 ubiquitin ligases which facilitate the ubiquitination of different proteins in vivo, which can be divided into four families: HECT-domain E3s, U-box E3s, monomeric RING E3s and multi-subunit E3s. See generally Li et al. (PLOS One, 2008, 3, 1487) titled “Genome-wide and functional annotation of human E3 ubiquitin ligases identifies MULAN, a mitochondrial E3 that regulates the organelle's dynamics and signaling.”; Bemdsen et al. (Nat. Struct. Mol. Biol., 2014, 21, 301-307) titled “New insights into ubiquitin E3 ligase mechanism”; Deshaies et al. (Ann. Rev. Biochem., 2009, 78, 399-434) titled “RING domain E3 ubiquitin ligases.”; Spratt et al. (Biochem. 2014, 458, 421-437) titled “RBR E3 ubiquitin ligases: new structures, new insights, new questions.”; and Wang et al. (Nat. Rev. Cancer., 2014, 14, 233-347) titled “Roles of F-box proteins in cancer.”

The UPP is used to induce selective protein degradation, including use of fusion proteins to artificially ubiquitinate target proteins and synthetic small-molecule probes to induce proteasome-dependent degradation. Bifunctional compounds composed of a target protein binding ligand and an E3 ubiquitin ligase ligand induce proteasome-mediated degradation of selected proteins via their recruitment to E3 ubiquitin ligase and subsequent ubiquitination. These drug-like molecules offer the possibility of temporal control over protein expression. Such compounds are capable of inducing the inactivation of a protein of interest upon addition to cells or administration to an animal or human, and could be useful as biochemical reagents and lead to a new paradigm for the treatment of diseases by removing pathogenic or oncogenic proteins (Crews C, Chemistry & Biology, 2010, 17(6):551-555; Schnnekloth J S Jr., Chembiochem, 2005, 6(1):40-46).

An ongoing need exists in the art for effective treatments for disease, especially cancers. As such, small molecule therapeutic agents that leverage UPP mediated protein degradation to target cancer-associated proteins such as cyclin-dependent kinase 2 (“CDK2”), cyclin E (“CCNE1” and/or “CCNE2”) or CDK2 and CCNE (CCNE1 and/or CCNE2) protein hold promise as therapeutic agents. Accordingly, there remains a need to find compounds that are CDK2 degraders, CCNE (CCNE1 and/or CCNE2) degraders or dual CDK2 and CCNE (CCNE1 and/or CCNE2) degraders useful as therapeutic agents.

### SUMMARY OF THE INVENTION

In an embodiment, provided is a compound of Formula A:

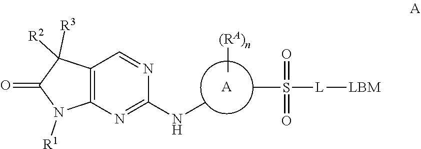

or a pharmaceutically acceptable salt thereof, wherein:
Ring A is phenyl, 3-14 membered cycloalkyl or 4-14 membered heterocyclyl containing 1-3 heteroatoms independently selected from N, O and S;
R¹ is selected from —C₁₋₆ alkyl, —C₂₋₆ alkenyl, —C₂₋₆ alkynyl, —C₁₋₆ haloalkyl, —C₁₋₆ heteroalkyl, —C₃₋₁₄ cycloalkyl, 6-14 membered aryl, 4-14 membered heterocyclyl, 5-14 membered heteroaryl, —C₁₋₄ alkyl-C₃₋₁₄ cycloalkyl, —C₁₋₄ alkyl-(6-14 membered aryl), —C₁₋₄ alkyl-(4-14 membered heterocyclyl), and —C₁₋₄ alkyl (5-14 membered heteroaryl), wherein each heterocyclyl and heteroaryl contains 1-3 heteroatoms independently selected from N, O and S, and wherein each alkyl, alkenyl, alkynyl, cycloalkyl, aryl, heterocyclyl, and heteroaryl is substituted with 0, 1, 2, 3 or 4 instances of R⁶;
each R² and R³ is independently selected from —C₁₋₆ alkyl, —C₁₋₆ haloalkyl, —C₁₋₆ heteroalkyl, —C₂₋₆ alkenyl, —C₂₋₆ alkynyl, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, and wherein each alkyl, alkenyl, alkynyl, cycloalkyl, aryl, heterocyclyl, and heteroaryl is substituted with 0, 1, 2, 3 or 4 instances of R^{B};
or R² and R³, together with the carbon atom to which they are attached, form Ring B, wherein Ring B is a 3-7 membered cycloalkyl ring or a 4-7 membered heterocyclyl ring containing 1 or 3 heteroatoms independently selected from N, O and S, wherein Ring B is substituted with 0, 1, 2, 3, or 4 instances of R^{B};
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(═O)—, —C(═O)O—, —C(═O)—, —S(═O)—, —S(═O)₂—, —NRS(═O)₂—, —S(═O)₂NR—, —NRC(═O)—, —C(═O)NR—, —OC(═O)NR— or —NRC(═O)O—, wherein:
each -Cy- is independently a bivalent ring selected from phenylene, an 8-10 membered bicyclic arylene, a 4-7 membered monocyclic carbocyclylene, a 5-11 membered spiro carbocyclylene, a 4-10 membered bicyclic carbocyclylene, a 5-10 membered bridged carbocyclylene, a 4-7 membered monocyclic heterocyclylene having 1-2 heteroatoms independently selected from nitrogen, oxygen, and sulfur, a 5-11 membered spiro heterocyclylene having 1-2 heteroatoms independently selected from nitrogen, oxygen, and sulfur, an 4-10 membered bicyclic heterocyclylene having 1-2 heteroatoms independently selected from nitrogen, oxygen, and sulfur, a 5-10 membered bridged bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur, wherein each phenylene, arylene, carbocyclylene, heterocyclylene and heteroarylene is substituted with 0, 1, 2, 3, or 4 instances of R^{C};
LBM is selected from

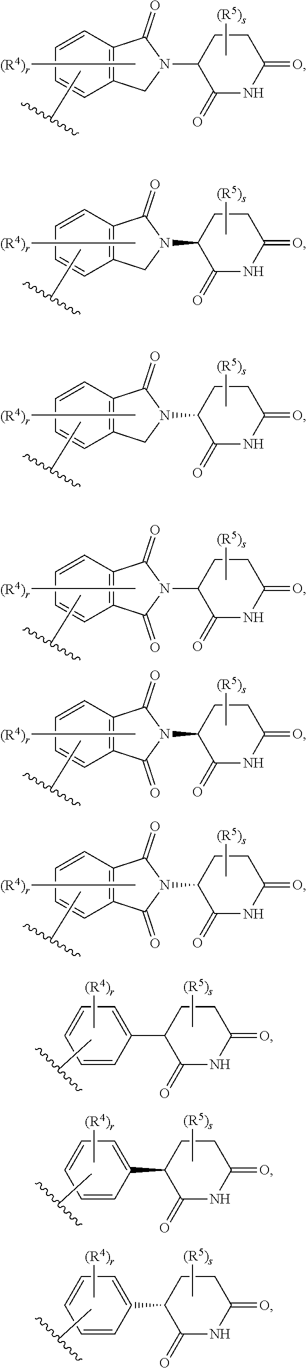

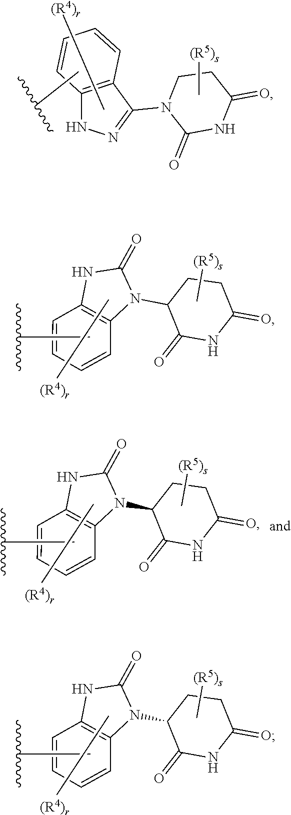

each instance of R^{A}, R^{B}, R^{C}, R⁴, R⁵ and R⁶ is independently selected from oxo, deuterium, halogen, —C₁₋₆ alkyl, —C₁₋₆ heteroalkyl, —C₁₋₆ haloalkyl, —C₂₋₆ alkenyl, —C₂₋₆ alkynyl, —CN, —NO₂, —OR, —SR, —NR₂, —S(O)₂R, —S(O)₂NR₂, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S;
each instance of R is independently hydrogen, —C₁₋₆ alkyl, —C₁₋₆ haloalkyl, —C₁₋₆ heteroalkyl, —C₂₋₆ alkenyl, —C₂₋₆ alkynyl, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, or two R groups attached to the same nitrogen are optionally taken together with nitrogen to which they are attached to form an optionally substituted 4-7 heterocyclyl having 0, 1 or 2 additional heteroatoms independently selected from N, O and S;
n is 0, 1, 2, 3, or 4;
r is 0, 1, 2, 3, or 4; and
s is 0, 1, 2, 3, or 4.

In an embodiment, provided is a compound of Formula I or Formula I-1:

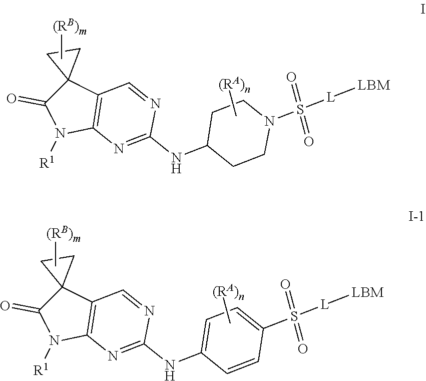

or a pharmaceutically acceptable salt thereof, wherein:
R¹ is selected from —C₁₋₆ alkyl, —C₂₋₆ alkenyl, —C₂₋₆ alkynyl, —C₁₋₆ haloalkyl, —C₁₋₆ heteroalkyl, —C₃₋₁₄ cycloalkyl, 6-14 membered aryl, 4-14 membered heterocyclyl, 5-14 membered heteroaryl, —C₁₋₄ alkyl-C₃₋₁₄ cycloalkyl, —C₁₋₄ alkyl-(6-14 membered aryl), —C₁₋₄ alkyl-(4-14 membered heterocyclyl), and —C₁₋₄ alkyl (5-14 membered heteroaryl), wherein each heterocyclyl and heteroaryl contains 1-3 heteroatoms independently selected from N, O and S, and wherein each alkyl, alkenyl, alkynyl, cycloalkyl, aryl, heterocyclyl, and heteroaryl is substituted with 0, 1, 2, 3 or 4 instances of R⁶;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(═O)—, —C(═O)O—, —C(═O)—, —S(═O)—, —S(═O)₂—, —NRS(═O)₂—, —S(═O)₂NR—, —NRC(═O)—, —C(═O)NR—, —OC(═O)NR— or —NRC(═O)O—, wherein:
each -Cy- is independently a bivalent ring selected from phenylene, an 8-10 membered bicyclic arylene, a 4-7 membered monocyclic carbocyclylene, a 5-11 membered spiro carbocyclylene, a 4-10 membered bicyclic carbocyclylene, a 5-10 membered bridged carbocyclylene, a 4-7 membered monocyclic heterocyclylene having 1-2 heteroatoms independently selected from nitrogen, oxygen, and sulfur, a 5-11 membered spiro heterocyclylene having 1-2 heteroatoms independently selected from nitrogen, oxygen, and sulfur, an 4-10 membered bicyclic heterocyclylene having 1-2 heteroatoms independently selected from nitrogen, oxygen, and sulfur, a 5-10 membered bridged bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur, wherein each phenylene, arylene, carbocyclylene, heterocyclylene and heteroarylene is substituted with 0, 1, 2, 3, or 4 instances of R^{C};
   LBM is selected from

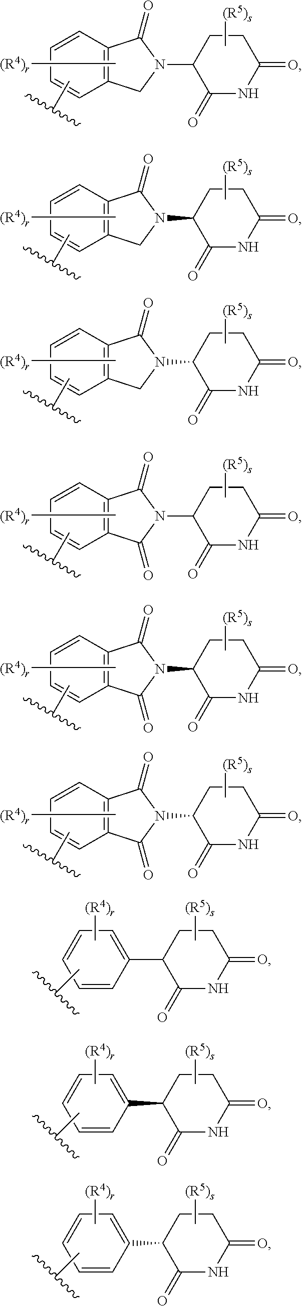

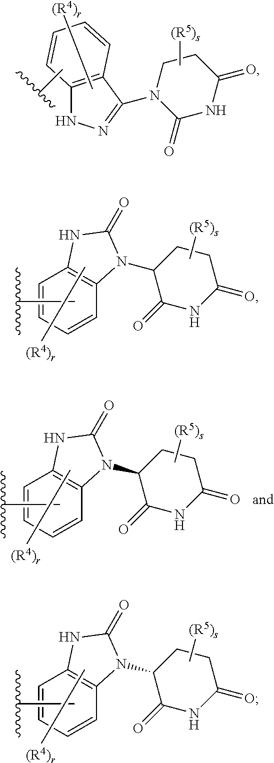

each instance of R^{A}, R^{B}, R^{C}, R⁴, R⁵ and R⁶ is independently selected from oxo, deuterium, halogen, —C₁₋₆ alkyl, —C₁₋₆ heteroalkyl, —C₁₋₆ haloalkyl, —C₂₋₆ alkenyl, —C₂₋₆ alkynyl, —CN, —NO₂, —OR, —SR, —NR₂, —S(O)₂R, —S(O)₂NR₂, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S;
each instance of R is independently hydrogen, —C₁₋₆ alkyl, —C₁₋₆ haloalkyl, —C₁₋₆ heteroalkyl, —C₂₋₆ alkenyl, —C₂₋₆ alkynyl, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, or two R groups attached to the same nitrogen are optionally taken together with nitrogen to which they are attached to form an optionally substituted 4-7 heterocyclyl having 0, 1 or 2 additional heteroatoms independently selected from N, O and S;
m is 0, 1, 2, 3, or 4;
n is 0, 1, 2, 3, or 4;
r is 0, 1, 2, 3, or 4; and
s is 0, 1, 2, 3, or 4.

In an embodiment, provided is a pharmaceutical composition comprising a compound of Formula A, Formula I or Formula I-1 as described herein, or a pharmaceutically acceptable salt thereof, and a pharmaceutically acceptable carrier, adjuvant, or diluent.

In an embodiment, provided is a method of inhibiting CDK2 and/or CCNE (CCNE1 and/or CCNE2) signaling in a sample, e.g., in vivo or in vitro, by contacting CDK2 and/or CCNE (CCNE1 and/or CCNE2) with a compound or pharmaceutically acceptable salt thereof or composition as described herein.

In an embodiment, provided is a use of a compound or pharmaceutically acceptable salt thereof or composition as described herein in a method of inhibiting CDK2 and/or CCNE (CCNE1 and/or CCNE2) signaling in a sample, e.g., in vivo or in vitro, by contacting CDK2 and/or CCNE (CCNE1 and/or CCNE2) with the compound or composition.

In an embodiment, provided is a use of a compound or pharmaceutically acceptable salt thereof or composition as described herein in the manufacturing of a medicament for inhibiting CDK2 and/or CCNE (CCNE1 and/or CCNE2) signaling in a sample, e.g., in vivo or in vitro.

In an embodiment, provided is a compound or pharmaceutically acceptable salt thereof or composition as described herein for use in a method of inhibiting CDK2 and/or CCNE (CCNE1 and/or CCNE2) signaling in a sample, e.g., in vivo or in vitro, by contacting CDK2 and/or CCNE (CCNE1 and/or CCNE2) with the compound or composition.

In an embodiment, provided is a compound or pharmaceutically acceptable salt thereof or composition as described herein for use in manufacturing of a medicament for inhibiting CDK2 and/or CCNE (CCNE1 and/or CCNE2) signaling in a sample, e.g., in vivo or in vitro, by contacting CDK2 and/or CCNE (CCNE1 and/or CCNE2) with the compound or composition.

In an embodiment, provided is a method of inhibiting CDK2 signaling in a sample, e.g., in vivo or in vitro, by contacting CDK2 with a compound or pharmaceutically acceptable salt thereof or composition as described herein.

In an embodiment, provided is a use of a compound or pharmaceutically acceptable salt thereof or composition as described herein in a method of inhibiting CDK2 signaling in a sample, e.g., in vivo or in vitro, by contacting CDK2 with a compound or pharmaceutically acceptable salt thereof or composition as described herein.

In an embodiment, provided is a use of a compound or pharmaceutically acceptable salt thereof or composition as described herein in the manufacturing of a medicament for inhibiting CDK2 signaling in a sample, e.g., in vivo or in vitro.

In an embodiment, provided is a compound or pharmaceutically acceptable salt thereof or composition as described herein for use in a method of inhibiting CDK2 signaling in a sample, e.g., in vivo or in vitro, by contacting CDK2 with a compound or pharmaceutically acceptable salt thereof or composition as described herein.

In an embodiment, provided is a compound or pharmaceutically acceptable salt thereof or composition as described herein for use in the manufacturing of a medicament for inhibiting CDK2 signaling in a sample, e.g., in vivo or in vitro.

In an embodiment, provided is a method of inhibiting CCNE (CCNE1 and/or CCNE2) signaling in a sample, e.g., in vivo or in vitro, by contacting CCNE (CCNE1 and/or CCNE2) with a compound or pharmaceutically acceptable salt thereof or composition as described herein.

In an embodiment, provided is a use of a compound or pharmaceutically acceptable salt thereof or composition as described herein in a method of inhibiting CCNE (CCNE1 and/or CCNE2) signaling in a sample, e.g., in vivo or in vitro, by contacting CCNE (CCNE1 and/or CCNE2) with a compound or pharmaceutically acceptable salt thereof or composition as described herein.

In an embodiment, provided is a use of a compound or pharmaceutically acceptable salt thereof or composition as described herein in the manufacturing of a medicament for inhibiting CCNE (CCNE1 and/or CCNE2) signaling in a sample, e.g., in vivo or in vitro.

In an embodiment, provided is a compound or pharmaceutically acceptable salt thereof or composition as described herein for use in a method of inhibiting CCNE (CCNE1 and/or CCNE2) signaling in a sample, e.g., in vivo or in vitro, by contacting CCNE (CCNE1 and/or CCNE2) with a compound or pharmaceutically acceptable salt thereof or composition as described herein.

In an embodiment, provided is a compound or pharmaceutically acceptable salt thereof or composition as described herein for use in the manufacturing of a medicament for inhibiting CCNE (CCNE1 and/or CCNE2) signaling in a sample, e.g., in vivo or in vitro.

In an embodiment, provided is a method of inhibiting CDK2 and CCNE (CCNE1 and/or CCNE2) signaling in a sample, e.g., in vivo or in vitro, by contacting CDK2 and/or CCNE (CCNE1 and/or CCNE2) with a compound or pharmaceutically acceptable salt thereof or composition as described herein.

In an embodiment, provided is a use of a compound or pharmaceutically acceptable salt thereof or composition as described herein in a method of inhibiting CDK2 and CCNE (CCNE1 and/or CCNE2) signaling in a sample, e.g., in vivo or in vitro, by contacting CDK2 and/or CCNE (CCNE1 and/or CCNE2) with a compound or pharmaceutically acceptable salt thereof or composition as described herein.

In an embodiment, provided is a use of a compound or pharmaceutically acceptable salt thereof or composition as described herein in the manufacturing of a medicament for inhibiting CDK2 and CCNE (CCNE1 and/or CCNE2) signaling in a sample, e.g., in vivo or in vitro.

In an embodiment, provided is a compound or pharmaceutically acceptable salt thereof or composition as described herein for use in a method of inhibiting CDK2 and CCNE (CCNE1 and/or CCNE2) signaling in a sample, e.g., in vivo or in vitro, by contacting CDK2 and/or CCNE (CCNE1 and/or CCNE2) with a compound or pharmaceutically acceptable salt thereof or composition as described herein.

In an embodiment, provided is a compound or pharmaceutically acceptable salt thereof or composition as described herein for use in the manufacturing of a medicament for inhibiting CDK2 and CCNE (CCNE1 and/or CCNE2) signaling in a sample, e.g., in vivo or in vitro.

In an embodiment, provided is a method of treating a CDK2 and/or CCNE (CCNE1 and/or CCNE2)-mediated disorder in a patient in need thereof, comprising administering to the patient a compound or pharmaceutically acceptable salt thereof or composition as described herein.

In an embodiment, provided is a use of a compound or pharmaceutically acceptable salt thereof or composition as described herein in a method of treating a CDK2 and/or CCNE (CCNE1 and/or CCNE2)-mediated disorder in a patient in need thereof, comprising administering to the patient a compound or pharmaceutically acceptable salt thereof or composition as described herein.

In an embodiment, provided is a use of a compound or pharmaceutically acceptable salt thereof or composition as described herein in the manufacturing of a medicament for treating a CDK2 and/or CCNE (CCNE1 and/or CCNE2)-mediated disorder in a patient in need thereof.

In an embodiment, provided is a compound or pharmaceutically acceptable salt thereof or composition as described herein for use in a method of treating a CDK2 and/or CCNE (CCNE1 and/or CCNE2)-mediated disorder in a patient in need thereof, comprising administering to the patient a compound or pharmaceutically acceptable salt thereof or composition as described herein.

In an embodiment, provided is a compound or pharmaceutically acceptable salt thereof or composition as described herein for use in the manufacturing of a medicament for treating a CDK2 and/or CCNE (CCNE1 and/or CCNE2)-mediated disorder in a patient in need thereof.

In an embodiment, provided is a method of treating a CDK2-mediated disorder in a patient in need thereof, comprising administering to the patient a compound or pharmaceutically acceptable salt thereof or composition as described herein.

In an embodiment, provided is a use of a compound or pharmaceutically acceptable salt thereof or composition as described herein in a method of treating a CDK2-mediated disorder in a patient in need thereof, comprising administering to the patient a compound or pharmaceutically acceptable salt thereof or composition as described herein.

In an embodiment, provided is a use of a compound or pharmaceutically acceptable salt thereof or composition as described herein in the manufacturing of a medicament for treating a CDK2-mediated disorder in a patient in need thereof.

In an embodiment, provided is a compound or pharmaceutically acceptable salt thereof or composition as described herein for use in a method of treating a CDK2-mediated disorder in a patient in need thereof, comprising administering to the patient a compound or pharmaceutically acceptable salt thereof or composition as described herein.

In an embodiment, provided is a compound or pharmaceutically acceptable salt thereof or composition as described herein for use in the manufacturing of a medicament for treating a CDK2-mediated disorder in a patient in need thereof.

In an embodiment, provided is a method of treating a CCNE (CCNE1 and/or CCNE2)-mediated disorder in a patient in need thereof, comprising administering to the patient a compound or pharmaceutically acceptable salt thereof or composition as described herein.

In an embodiment, provided is a use of a compound or pharmaceutically acceptable salt thereof or composition as described herein in a method of treating a CCNE (CCNE1 and/or CCNE2)-mediated disorder in a patient in need thereof, comprising administering to the patient a compound or pharmaceutically acceptable salt thereof or composition as described herein.

In an embodiment, provided is a use of a compound or pharmaceutically acceptable salt thereof or composition as described herein in the manufacturing of a medicament for treating a CCNE (CCNE1 and/or CCNE2)-mediated disorder in a patient in need thereof.

In an embodiment, provided is a compound or pharmaceutically acceptable salt thereof or composition as described herein for use in a method of treating a CCNE (CCNE1 and/or CCNE2)-mediated disorder in a patient in need thereof, comprising administering to the patient a compound or pharmaceutically acceptable salt thereof or composition as described herein.

In an embodiment, provided is a compound or pharmaceutically acceptable salt thereof or composition as described herein for use in the manufacturing of a medicament for treating a CCNE (CCNE1 and/or CCNE2)-mediated disorder in a patient in need thereof.

In an embodiment, provided is a method of treating a CDK2 and CCNE (CCNE1 and/or CCNE2)-mediated disorder in a patient in need thereof, comprising administering to the patient a compound or pharmaceutically acceptable salt thereof or composition as described herein.

In an embodiment, provided is a use of a compound or pharmaceutically acceptable salt thereof or composition as described herein in a method of treating a CDK2 and CCNE (CCNE1 and/or CCNE2)-mediated disorder in a patient in need thereof, comprising administering to the patient a compound or pharmaceutically acceptable salt thereof or composition as described herein.

In an embodiment, provided is a use of a compound or pharmaceutically acceptable salt thereof or composition as described herein in the manufacturing of a medicament for treating a CDK2 and CCNE (CCNE1 and/or CCNE2)-mediated disorder in a patient in need thereof.

In an embodiment, provided is compound or pharmaceutically acceptable salt thereof or composition as described herein for use in a method of treating a CDK2 and CCNE (CCNE1 and/or CCNE2)-mediated disorder in a patient in need thereof, comprising administering to the patient a compound or pharmaceutically acceptable salt thereof or composition as described herein.

In an embodiment, provided is a compound or pharmaceutically acceptable salt thereof or composition as described herein for use in the manufacturing of a medicament for treating a CDK2 and CCNE (CCNE1 and/or CCNE2)-mediated disorder in a patient in need thereof.

### DETAILED DESCRIPTION

### Definitions

As used in the present disclosure, the following words and phrases are generally intended to have the meanings as set forth below unless expressly indicated otherwise or the context in which they are used indicates otherwise.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein are constructed according to the standard rules of chemical valency known in the chemical arts.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. Where elements are presented as lists, e.g., in Markush group format, each subgroup of the elements is also disclosed, and any element(s) can be removed from the group. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., to at least one) of the grammatical object of the article, unless the context is inappropriate. By way of example, in certain contexts, “an element” means one element and/or in certain contexts more than one element. By way of another example, in certain contexts “a compound” means one compound and/or in certain contexts more than one compound (e.g., a mixture of two or more compounds).

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred from the context.

At various places in the present specification, variables or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### Chemical Definitions

These and other exemplary substituents are described in more detail in the Detailed Description, Examples, and Claims. The invention is not intended to be limited in any manner by the above exemplary listing of substituents.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). Additionally encompassed are compounds described herein as individual isomers substantially free of other isomers, and alternatively, as mixtures of various isomers.

The “enantiomeric excess” (“e.e.”) or “% enantiomeric excess” (“% e.e.”) of a composition as used herein refers to an excess of one enantiomer relative to the other enantiomer present in the composition. For example, a composition can contain 90% of one enantiomer, e.g., the S enantiomer, and 10% of the other enantiomer, i.e., the R enantiomer.

$$e . e . = ( 90 - 10 ) / 100 = 80 \% .$$

Thus, a composition containing 90% of one enantiomer and 10% of the other enantiomer is said to have an enantiomeric excess of 80%.

The “diastereomeric excess” (“d.e.”) or “% diastereomeric excess” (“% d.e.”) of a composition as used herein refers to an excess of one diastereomer relative to one or more different diastereomers present in the composition. For example, a composition can contain 90% of one diastereomer, and 10% of one or more different diastereomers.

$$d . e . = ( 90 - 10 ) / 100 = 80 \% .$$

Thus, a composition containing 90% of one diastereomers and 10% of one or more different diastereomers is said to have a diastereomeric excess of 80%.

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

In a formula, [symbol] is a single bond where the stereochemistry of the moieties immediately attached thereto is not specified.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e. at least one) of the grammatical objects of the article. By way of example “an analogue” means one analogue or more than one analogue.

The term “unsaturated bond” refers to a double or triple bond.

The term “unsaturated” or “partially unsaturated” refers to a moiety that includes at least one double or triple bond.

The term “saturated” refers to a moiety that does not contain a double or triple bond, i.e., the moiety only contains single bonds.

Affixing the suffix “-ene” to a group indicates the group is a divalent moiety, e.g. alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

The term “azido” refers to the radical —N₃.

“Aliphatic” refers to an alkyl, alkenyl, alkynyl, or carbocyclyl group, as defined herein.

“Cycloalkylalkyl” refers to an alkyl radical in which the alkyl group is substituted with a cycloalkyl group. Typical cycloalkylalkyl groups include, but are not limited to, cyclopropylmethyl, cyclobutylmethyl, cyclopentylmethyl, cyclohexylmethyl, cycloheptylmethyl, cyclooctylmethyl, cyclopropylethyl, cyclobutylethyl, cyclopentylethyl, cyclohexylethyl, cycloheptylethyl, and cyclooctylethyl, and the like.

“Heterocyclylalkyl” refers to an alkyl radical in which the alkyl group is substituted with a heterocyclyl group. Typical heterocyclylalkyl groups include, but are not limited to, pyrrolidinylmethyl, piperidinylmethyl, piperazinylmethyl, morpholinylmethyl, pyrrolidinylethyl, piperidinylethyl, piperazinylethyl, morpholinylethyl, and the like.

“Aralkyl” or “arylalkyl” is a subset of alkyl and aryl, as defined herein, and refers to an optionally substituted alkyl group substituted by an optionally substituted aryl group.

“Alkyl” refers to a radical of a straight-chain or branched saturated hydrocarbon group having from 1 to 20 carbon atoms (“C₁₋₂₀ alkyl”). In an embodiment, an alkyl group has 1 to 12 carbon atoms (“C₁₋₁₂ alkyl”). In an embodiment, an alkyl group has 1 to 10 carbon atoms (“C₁₋₁₀ alkyl”). In an embodiment, an alkyl group has 1 to 9 carbon atoms (“C₁₋₉ alkyl”). In an embodiment, an alkyl group has 1 to 8 carbon atoms (“C₁₋₈ alkyl”). In an embodiment, an alkyl group has 1 to 7 carbon atoms (“C₁₋₇ alkyl”). In an embodiment, an alkyl group has 1 to 6 carbon atoms (“C₁₋₆ alkyl”, also referred to herein as “lower alkyl”). In an embodiment, an alkyl group has 1 to 5 carbon atoms (“C₁₋₅ alkyl”). In an embodiment, an alkyl group has 1 to 4 carbon atoms (“C₁₋₄ alkyl”). In an embodiment, an alkyl group has 1 to 3 carbon atoms (“C₁₋₃ alkyl”). In an embodiment, an alkyl group has 1 to 2 carbon atoms (“C₁₋₂ alkyl”). In an embodiment, an alkyl group has 1 carbon atom (“C₁ alkyl”). In an embodiment, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₅). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), ⁱPr (—CH(CH₃)₂), ⁿPr (—CH₂CH₂CH₃), ⁿBu (—CH₂CH₂CH₂CH₃), or ^{t}Bu (—CH₂CH(CH₃)₂).

“Alkylene” refers to an alkyl group wherein two hydrogens are removed to provide a divalent radical, and which may be substituted or unsubstituted. Unsubstituted alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted alkylene groups, e.g., substituted with one or more alkyl (methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), and the like. When a range or number of carbons is provided for a particular alkylene group, it is understood that the range or number refers to the range or number of carbons in the linear carbon divalent chain. Alkylene groups may be substituted or unsubstituted with one or more substituents as described herein.

“Alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g. 1, 2, 3, or 4 carbon-carbon triple bonds) (“C₂₋₂₀ alkenyl”). In certain embodiments, alkenyl does not contain any triple bonds. In an embodiment, an alkenyl group has 2 to 10 carbon atoms (“C₂₋₁₀ alkenyl”). In an embodiment, an alkenyl group has 2 to 9 carbon atoms (“C₂₋₉ alkenyl”). In an embodiment, an alkenyl group has 2 to 8 carbon atoms (“C₂₋₈ alkenyl”). In an embodiment, an alkenyl group has 2 to 7 carbon atoms (“C₂₋₇ alkenyl”). In an embodiment, an alkenyl group has 2 to 6 carbon atoms (“C₂₋₆ alkenyl”). In an embodiment, an alkenyl group has 2 to 5 carbon atoms (“C₂₋₅ alkenyl”). In an embodiment, an alkenyl group has 2 to 4 carbon atoms (“C₂₋₄ alkenyl”). In an embodiment, an alkenyl group has 2 to 3 carbon atoms (“C₂₋₃ alkenyl”). In an embodiment, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In an embodiment, an alkynyl group has 2 to 10 carbon atoms (“C₂₋₁₀ alkynyl”). In an embodiment, an alkynyl group has 2 to 9 carbon atoms (“C₂₋₉ alkynyl”). In an embodiment, an alkynyl group has 2 to 8 carbon atoms (“C₂₋₈ alkynyl”). In an embodiment, an alkynyl group has 2 to 7 carbon atoms (“C₂₋₇ alkynyl”). In an embodiment, an alkynyl group has 2 to 6 carbon atoms (“C₂₋₆ alkynyl”). In an embodiment, an alkynyl group has 2 to 5 carbon atoms (“C₂₋₅ alkynyl”). In an embodiment, an alkynyl group has 2 to 4 carbon atoms (“C₂₋₄ alkynyl”). In an embodiment, an alkynyl group has 2 to 3 carbon atoms (“C₂₋₃ alkynyl”). In an embodiment, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In an embodiment, a heteroalkyl group is a saturated group having 1 to 9 carbon atoms and 1, 2, 3, or 4 heteroatoms (“heteroC₁₋₉ alkyl”). In an embodiment, a heteroalkyl group is a saturated group having 1 to 8 carbon atoms and 1, 2, 3, or 4 heteroatoms (“heteroC₁₋₈ alkyl”). In an embodiment, a heteroalkyl group is a saturated group having 1 to 7 carbon atoms and 1, 2, 3, or 4 heteroatoms (“heteroC₁₋₇ alkyl”). In an embodiment, a heteroalkyl group is a group having 1 to 6 carbon atoms and 1, 2, or 3 heteroatoms (“heteroC₁₋₆ alkyl”). In an embodiment, a heteroalkyl group is a saturated group having 1 to 5 carbon atoms and 1 or 2 heteroatoms (“heteroC₁₋₅ alkyl”). In an embodiment, a heteroalkyl group is a saturated group having 1 to 4 carbon atoms and 1 or 2 heteroatoms (“heteroC₁₋₄ alkyl”). In an embodiment, a heteroalkyl group is a saturated group having 1 to 3 carbon atoms and 1 heteroatom (“heteroC₁₋₃ alkyl”). In an embodiment, a heteroalkyl group is a saturated group having 1 to 2 carbon atoms and 1 heteroatom (“heteroC₁₋₂ alkyl”). In an embodiment, a heteroalkyl group is a saturated group having 1 carbon atom and 1 heteroatom (“heteroC₁ alkyl”). In an embodiment, a heteroalkyl group is a saturated group having 2 to 6 carbon atoms and 1 or 2 heteroatoms (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl. Exemplary heteroalkyl groups include: —CH₂OH, —CH₂OCH₃, —CH₂NH₂, —CH₂NH(CH₃), —CH₂N(CH₃)₂, —CH₂CH₂OH, —CH₂CH₂OCH₃, —CH₂CH₂NH₂, —CH₂CH₂NH(CH₃), —CH₂CH₂N(CH₃)₂.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In an embodiment, an aryl group has six ring carbon atoms (“C₆ aryl”; e.g. phenyl). In an embodiment, an aryl group has ten ring carbon atoms (“C₁₀ aryl”; e.g., naphthyl such as 1-naphthyl and 2-naphthyl). In an embodiment, an aryl group has fourteen ring carbon atoms (“C₁₋₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Particularly aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

In certain embodiments, an aryl group is substituted with one or more of groups selected from halo, C₁-C₈ alkyl, C₁-C₈ haloalkyl, cyano, hydroxy, C₁-C₈ alkoxy, and amino.

Examples of representative substituted aryls include the following

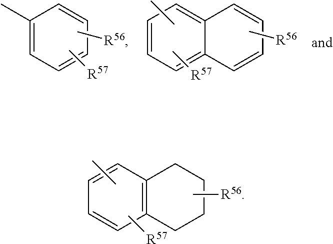

wherein one of R⁵⁶ and R⁵⁷ may be hydrogen and at least one of R⁵⁶ and R⁵⁷ is each independently selected from C₁-C₈ alkyl, C₁-C₈ haloalkyl, 4-10 membered heterocyclyl, alkanoyl, C₁-C₈ alkoxy, heteroaryloxy, alkylamino, arylamino, heteroarylamino, —NR⁵⁸COR⁵⁹, —NR⁵⁸SOR⁵⁹NR⁵⁸SO₂R⁵⁹, —COOalkyl, —COOaryl, —CONR⁵⁸R⁵⁹, —CONR⁵⁸OR⁵⁹, —NR⁵⁸R⁵⁹, —SO₂NR⁵⁸R⁵⁹, —S-alkyl, —SOalkyl, —SO₂alkyl, —Saryl, —SOaryl, —SO₂aryl; or R⁵⁶ and R⁵⁷ may be joined to form a cyclic ring (saturated or unsaturated) from 5 to 8 atoms, optionally containing one or more heteroatoms selected from the group consisting of N, O, or S. R⁶⁰ and R⁶¹ are independently hydrogen, —C₁-C₈ alkyl, —C₁-C₄haloalkyl, —C₃-C₁₀ cycloalkyl, 4-10 membered heterocyclyl, C₆-C₁₀ aryl, substituted C₆-C₁₀ aryl, 5-10 membered heteroaryl, or substituted 5-10 membered heteroaryl.

“Fused aryl” refers to an aryl having two of its ring carbons in common with a second aryl or heteroaryl ring or with a carbocyclyl or heterocyclyl ring.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, In such instances, unless otherwise specified, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In an embodiment, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In an embodiment, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In an embodiment, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In an embodiment, the 5-6 membered heteroaryl has 1-3 ring heteroatoms selected from nitrogen, oxygen, and sulfur. In an embodiment, the 5-6 membered heteroaryl has 1-2 ring heteroatoms selected from nitrogen, oxygen, and sulfur. In an embodiment, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl. In an embodiment, a heteroaryl group is a bicyclic 8-12 membered aromatic ring system having ring carbon atoms and 1-6 ring heteroatoms provided in the aromatic ring system, wherein each heteroatom is independently selected from nitrogen, oxygen, and sulfur (“8-12 membered bicyclic heteroaryl”). In an embodiment, a heteroaryl group is an 8-10 membered bicyclic aromatic ring system having ring carbon atoms and 1-6 ring heteroatoms provided in the aromatic ring system, wherein each heteroatom is independently selected from nitrogen, oxygen, and sulfur (“8-10 membered bicyclic heteroaryl”). In an embodiment, a heteroaryl group is a 9-10 membered bicyclic aromatic ring system having ring carbon atoms and 1-6 ring heteroatoms provided in the aromatic ring system, wherein each heteroatom is independently selected from nitrogen, oxygen, and sulfur (“9-10 membered bicyclic heteroaryl”). Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following:

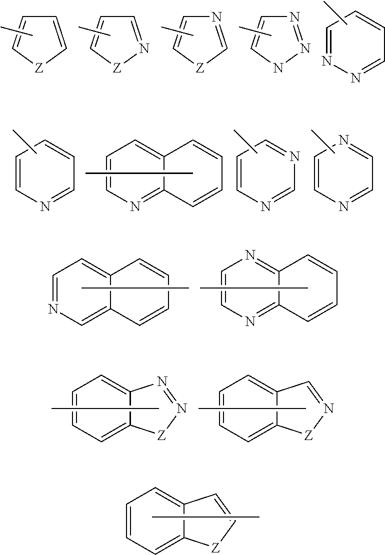

wherein each Z is selected from carbonyl, N, NR⁶⁵, O, and S; and R⁶⁵ is independently hydrogen, —C₁-C₈ alkyl, C₃-C₁₀ cycloalkyl, 4-10 membered heterocyclyl, C₆-C₁₀ aryl, and 5-10 membered heteroaryl.

In the structures described herein, a substituent attached to a polycyclic (e.g., bicyclic or tricyclic) cycloalkyl, heterocyclyl, aryl or heteroaryl with a bond that spans two or more rings is understood to mean that the substituent can be attached at any position in each of the rings.

“Heteroaralkyl” or “heteroarylalkyl” is a subset of “alkyl” and refers to an alkyl group substituted by a heteroaryl group, wherein the point of attachment is on the alkyl moiety.

The term “carbocyclyl” or “carbocyclic” refers to a radical of a non-aromatic monocyclic, bicyclic, or tricyclic or polycyclic hydrocarbon ring system having from 3 to 14 ring carbon atoms (“C₃₋₁₄ carbocyclyl”) and zero heteroatoms in the non-aromatic ring system. Carbocyclyl groups include fully saturated ring systems (e.g., cycloalkyls), and partially saturated ring systems. In an embodiment, a carbocyclyl group has 3 to 10 ring carbon atoms (“C₃₋₁₀ carbocyclyl”). In an embodiment, a carbocyclyl group has 3 to 8 ring carbon atoms (“C₃₋₈ carbocyclyl”). In an embodiment, a carbocyclyl group has 3 to 7 ring carbon atoms (“C₃₋₇ carbocyclyl”). In an embodiment, a carbocyclyl group has 3 to 6 ring carbon atoms (“C₃₋₆ carbocyclyl”). In an embodiment, a carbocyclyl group has 4 to 6 ring carbon atoms (“C₄₋₆ carbocyclyl”). In an embodiment, a carbocyclyl group has 5 to 6 ring carbon atoms (“C₅₋₆ carbocyclyl”). In an embodiment, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like.

As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

The term “cycloalkyl” as employed herein includes saturated cyclic, bicyclic, tricyclic, or polycyclic hydrocarbon groups having 3 to 14 carbons containing the indicated number of rings and carbon atoms (for example a C₃-C₁₄ monocyclic, C₄-C₁₄ bicyclic, C₅-C₁₄ tricyclic, or C₆-C₁₄ polycyclic cycloalkyl). In an embodiment “cycloalkyl” is a monocyclic cycloalkyl. In an embodiment, a monocyclic cycloalkyl has 3-14 ring carbon atoms. (“C₃₋₁₄ monocyclic cycloalkyl”). In an embodiment, a monocyclic cycloalkyl group has 3 to 10 ring carbon atoms (“C₃₋₁₀ monocyclic cycloalkyl”). In an embodiment, a monocyclic cycloalkyl group has 3 to 8 ring carbon atoms (“C₃₋₈ monocyclic cycloalkyl”). In an embodiment, a monocyclic cycloalkyl group has 3 to 6 ring carbon atoms (“C₃₋₆ monocyclic cycloalkyl”). In an embodiment, a monocyclic cycloalkyl group has 4 to 6 ring carbon atoms (“C₄₋₆ monocyclic cycloalkyl”). In an embodiment, a monocyclic cycloalkyl group has 5 to 6 ring carbon atoms (“C₅₋₆ monocyclic cycloalkyl”). In an embodiment, a monocyclic cycloalkyl group has 5 to 10 ring carbon atoms (“C₅₋₁₀ monocyclic cycloalkyl”). Examples of monocyclic C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈).

In an embodiment “cycloalkyl” is a bicyclic cycloalkyl. In an embodiment, a bicyclic cycloalkyl has 4-14 ring carbon atoms. (“C₄₋₁₄ bicyclic cycloalkyl”). In an embodiment, a bicyclic cycloalkyl group has 4 to 12 ring carbon atoms (“C₄₋₁₂ bicyclic cycloalkyl”). In an embodiment, a bicyclic cycloalkyl group has 4 to 10 ring carbon atoms (“C₄₋₁₀ bicyclic cycloalkyl”). In an embodiment, a bicyclic cycloalkyl group has 5 to 10 ring carbon atoms (“C₅₋₁₀ bicyclic cycloalkyl”). In an embodiment, a bicyclic cycloalkyl group has 6 to 10 ring carbon atoms (“C₆₋₁₀ bicyclic cycloalkyl”). In an embodiment, a bicyclic cycloalkyl group has 8 to 10 ring carbon atoms (“C₈₋₁₀ bicyclic cycloalkyl”). In an embodiment, a bicyclic cycloalkyl group has 7 to 9 ring carbon atoms (“C₇₋₉ bicyclic cycloalkyl”). Examples of bicyclic cycloalkyls include bicyclo[1.1.0]butane (C₄), bicyclo[1.1.1]pentane (C₅), spiro[2.2]pentane (C₅), bicyclo[2.1.0]pentane (C₅), bicyclo[2.1.1]hexane (C₆), bicyclo[3.1.0]hexane (C₆), spiro[2.3]hexane (C₆), bicyclo[2.2.1]heptane (norbornane) (C₇), bicyclo[3.2.0]heptane (C₇), bicyclo[3.1.1]heptane (C₇), bicyclo[3.1.1]heptane (C₇), bicyclo[4.1.0]heptane (C₇), spiro[2.4]heptane (C₇), spiro [3.3]heptane (C₇), bicyclo[2.2.2]octane (C₈), bicyclo[4.1.1]octane (C₈)octahydropentalene (C₈), bicyclo[3.2.1]octane (C₈), bicyclo[4.2.0]octane (C₈), spiro[2.5]octane (C₈), spiro[3.4]octane (C₈), bicyclo[3.3.1]nonane (C₉), octahydro-1H-indene (C₉), bicyclo[4.2.1]nonane (C₉), spiro[3.5]nonane (C₉), spiro[4.4]nonane (C₉), bicyclo[3.3.2]decane (C₁₀), bicyclo[4.3.1]decane (C₁₀), spiro[4.5]decane (C₁₀), bicyclo[3.3.3]undecane (C₁₁), decahydronaphthalene (C₁₀), bicyclo[4.3.2]undecane (C₁₁), spiro[5.5]undecane (C₁₁) and bicyclo[4.3.3]dodecane (C₁₂)

In an embodiment “cycloalkyl” is a tricyclic cycloalkyl. In an embodiment, a tricyclic cycloalkyl has 6-14 ring carbon atoms. (“C₆₋₁₄ tricyclic cycloalkyl”). In an embodiment, a tricyclic cycloalkyl group has 8 to 12 ring carbon atoms (“C₈₋₁₂ tricyclic cycloalkyl”). In an embodiment, a tricyclic cycloalkyl group has 10 to 12 ring carbon atoms (“C₁₀₋₁₂ tricyclic cycloalkyl. Examples of tricyclic cycloalkyls include adamantine (C₁₂). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In an embodiment, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In an embodiment, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In an embodiment, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In an embodiment, the 5-6 membered heterocyclyl has 1-3 ring heteroatoms selected from nitrogen, oxygen, and sulfur. In an embodiment, the 5-6 membered heterocyclyl has 1-2 ring heteroatoms selected from nitrogen, oxygen, and sulfur. In an embodiment, the 5-6 membered heterocyclyl has one ring heteroatom selected from nitrogen, oxygen, and sulfur.

Exemplary 3-membered heterocyclyl groups containing one heteroatom include, without limitation, aziridinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like. “Nitrogen-containing heterocyclyl” group means a 4- to 7-membered non-aromatic cyclic group containing at least one nitrogen atom, for example, but without limitation, morpholine, piperidine (e.g., 2-piperidinyl, 3-piperidinyl and 4-piperidinyl), pyrrolidine (e.g., 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl, cycloalkyl, e.g., heterocyclyl, aryl, e.g., heteroaryl, cycloalkenyl, e.g., cycloheteroalkenyl, and the like having from 1 to 5, and particularly from 1 to 3 heteroatoms.

“Acyl” refers to a radical —C(═O)R²⁰, where R²⁰ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. “Alkanoyl” is an acyl group wherein R²⁰ is a group other than hydrogen. Representative acyl groups include, but are not limited to, formyl (—CHO), acetyl (—C(═O)CH₃), cyclohexylcarbonyl, cyclohexylmethylcarbonyl, benzoyl (—C(═O)Ph), benzylcarbonyl (—C(═O)CH₂Ph), —C(═O)—C₁-C₈ alkyl, —C(═O)—(CH₂)ₜ(C₆-C₁₀ aryl), —C(═O)—(CH₂)ₜ(5-10 membered heteroaryl), —C(═O)—(CH₂)ₜ(C₃-C₁₀ cycloalkyl), and —C(═O)—(CH₂)ₜ(4-10 membered heterocyclyl), wherein t is an integer from 0 to 4. In certain embodiments, R²¹ is C₁-C₈ alkyl, substituted with halo or hydroxy; or C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

The term aminoalkyl refers to a substituted alkyl group wherein one or more of the hydrogen atoms are independently replaced by an —NH₂ group.

The term hydroxyalkyl refers to a substituted alkyl group wherein one or more of the hydrogen atoms are independently replaced by an —OH group.

The terms “alkylamino” and “dialkylamino” refer to —NH(alkyl) and —N(alkyl)₂ radicals, respectively. In an embodiment the alkylamino is a —NH(C₁-C₄ alkyl). In an embodiment the alkylamino is methylamino, ethylamino, propylamino, isopropylamino, n-butylamino, iso-butylamino, sec-butylamino or tert-butylamino. In an embodiment the dialkylamino is —N(C₁-C₆ alkyl)₂. In an embodiment the dialkylamino is a dimethylamino, a methylethylamino, a diethylamino, a methylpropylamino, a methylisopropylamino, a methylbutylamino, a methylisobutylamino or a methyltertbutylamino.

The term “aryloxy” refers to an —O-aryl radical. In an embodiment the aryloxy group is phenoxy.

The term “haloalkoxy” refers to alkoxy structures that are substituted with one or more halo groups or with combinations thereof. For example, the term “fluoroalkoxy” includes haloalkoxy groups, in which the halo is fluorine. In an embodiment haloalkoxy groups are difluoromethoxy and trifluoromethoxy.

“Alkoxy” refers to the group —OR²⁹ where R²⁹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e. with between 1 and 6 carbon atoms. Further particular alkoxy groups have between 1 and 4 carbon atoms.

In certain embodiments, R²⁹ is a group that has 1 or more substituents, for instance from 1 to 5 substituents, and particularly from 1 to 3 substituents, in particular 1 substituent, selected from the group consisting of amino, substituted amino, C₆-C₁₀ aryl, aryloxy, carboxyl, cyano, C₃-C₁₀ cycloalkyl, 4-10 membered heterocyclyl, halogen, 5-10 membered heteroaryl, hydroxyl, nitro, thioalkoxy, thioaryloxy, thiol, alkyl-S(O)—, aryl-S(O)—, alkyl-S(O)₂— and aryl-S(O)₂—. Exemplary ‘substituted alkoxy’ groups include, but are not limited to, —O—(CH₂)ₜ(C₆-C₁₀ aryl), —O—(CH₂)ₜ(5-10 membered heteroaryl), —O—(CH₂)ₜ(C₃-C₁₀ cycloalkyl), and —O—(CH₂)ₜ(4-10 membered heterocyclyl), wherein t is an integer from 0 to 4 and any aryl, heteroaryl, cycloalkyl or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Particular exemplary ‘substituted alkoxy’ groups are —OCF₃, —OCH₂CF₃, —OCH₂Ph, —OCH₂-cyclopropyl, —OCH₂CH₂OH, and —OCH₂CH₂N(CH₃)₂.

“Amino” refers to the radical —NH₂.

“Oxo group” refers to —C(═O)—.

“Substituted amino” refers to an amino group of the formula —N(R³⁸)₂ wherein R³⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or an amino protecting group, wherein at least one of R³⁸ is not a hydrogen. In certain embodiments, each R³⁸ is independently selected from hydrogen, —C₁-C₈ alkyl, —C₃-C₈ alkenyl, —C₃-C₈ alkynyl, C₆-C₁₀ aryl, 5-10 membered heteroaryl, 4-10 membered heterocyclyl, or C₃-C₁₀ cycloalkyl; or C₁-C₈ alkyl, substituted with halo or hydroxy; C₃-C₈ alkenyl, substituted with halo or hydroxy; C₃-C₈ alkynyl, substituted with halo or hydroxy, or —(CH₂)ₜ(C₆-C₁₀ aryl), —(CH₂)ₜ(5-10 membered heteroaryl), —(CH₂)ₜ(C₃-C₁₀ cycloalkyl), or —(CH₂)ₜ(4-10 membered heterocyclyl), wherein t is an integer between 0 and 8, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; or both R³⁸ groups are joined to form an alkylene group.

Exemplary “substituted amino” groups include, but are not limited to, —NR³⁹—C₁-C₈ alkyl, —NR³⁹—(CH₂)ₜ(C₆-C₁₀ aryl), —NR³⁹—(CH₂)ₜ(5-10 membered heteroaryl), —NR³⁹—(CH₂)ₜ(C₃-C₁₀ cycloalkyl), and —NR³⁹—(CH₂)ₜ(4-10 membered heterocyclyl), wherein t is an integer from 0 to 4, for instance 1 or 2, each R³⁹ independently represents H or C₁-C₈ alkyl; and any alkyl groups present, may themselves be substituted by halo, substituted or unsubstituted amino, or hydroxy; and any aryl, heteroaryl, cycloalkyl, or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. For the avoidance of doubt the term ‘substituted amino’ includes the groups alkylamino, substituted alkylamino, alkylarylamino, substituted alkylarylamino, arylamino, substituted arylamino, dialkylamino, and substituted dialkylamino as defined below. Substituted amino encompasses both monosubstituted amino and disubstituted amino groups.

In certain embodiments, the substituent present on the nitrogen atom is a nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂—CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —C₁₋₁₀ alkyl (e.g., aralkyl, heteroaralkyl), —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.
each instance of R^{aa} is, independently, selected from —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion.
each instance of R^{cc} is, independently, selected from hydrogen, —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OS₁(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, —C₁₋₆ alkyl, —C₁₋₆ perhaloalkyl, —C₂₋₆ alkenyl, —C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X is a counterion;
each instance of R^{ee} is, independently, selected from —C₁₋₆ alkyl, —C₁₋₆ perhaloalkyl, —C₂₋₆ alkenyl, —C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, —C₁₋₆ alkyl, —C₁₋₆ perhaloalkyl, —C₂₋₆ alkenyl, —C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃, —C(═S)N(C₁₋₆ alkyl)₂, —C(═S)NH(C₁₋₆ alkyl), —C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, —C₁₋₆ alkyl, —C₁₋₆ perhaloalkyl, —C₂₋₆ alkenyl, —C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluorenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl (o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate. Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), (3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R)₃⁺X⁻, —P(OR^{cc})₂, —P(OR)₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂—P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

The term “leaving group” is given its ordinary meaning in the art of synthetic organic chemistry and refers to an atom or a group capable of being displaced by a nucleophile. Examples of suitable leaving groups include, but are not limited to, halogen (such as F, —Cl, —Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In certain embodiments, the leaving group is halogen, alkanesulfonyloxy, arenesulfonyloxy, diazonium, alkyl diazenes, aryl diazenes, alkyl triazenes, aryl triazenes, nitro, alkyl nitrate, aryl nitrate, alkyl phosphate, aryl phosphate, alkyl carbonyl oxy, aryl carbonyl oxy, alkoxcarbonyl oxy, aryoxcarbonyl oxy ammonia, alkyl amines, aryl amines, hydroxyl group, alkyloxy group, or aryloxy. In some cases, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some cases, the leaving group is a brosylate, such as p-bromobenzenesulfonyloxy. In some cases, the leaving group is a nosylate, such as 2-nitrobenzenesulfonyloxy. In an embodiment, the leaving group is a sulfonate-containing group. In an embodiment, the leaving group is a tosylate group. The leaving group may also be a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

“Carboxy” refers to the radical —C(═O)OH.

“Cyano” refers to the radical —CN.

“Halo” or “halogen” refers to fluoro (F), chloro (Cl), bromo (Br), and iodo (I). In certain embodiments, the halo group is either fluoro or chloro.

“Haloalkyl” refers to an alkyl radical in which the alkyl group is substituted with one or more halogens. Typical haloalkyl groups include, but are not limited to, trifluoromethyl (—CF₃), difluoromethyl (—CHF₂), fluoromethyl (—CH₂F), chloromethyl (—CH₂Cl), dichloromethyl (—CHCl₂), tribromomethyl (—CH₂Br), and the like.

“Hydroxy” refers to the radical —OH.

“Nitro” refers to the radical —NO₂.

“Thioketo” refers to the group ═S.

Alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted,” whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. Any and all such combinations are contemplated in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —S(═O)(═NR^{bb})R^{aa}, OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), —C₁₋₁₀ alkyl, —C₁₋₁₀ haloalkyl, —C₂₋₁₀ alkenyl, —C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups; or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
each instance of R^{aa} is, independently, selected from C₁₋₁₀ alkyl, —C₁₋₁₀ haloalkyl, —C₂₋₁₀ alkenyl, —C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, —C₁₋₁₀ alkyl, —C₁₋₁₀ haloalkyl, —C₂₋₁₀ alkenyl, —C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, —C₁₋₁₀ alkyl, —C₁₋₁₀ haloalkyl, —C₂₋₁₀ alkenyl, —C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ee})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ee}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ee})₂, —OC(═NR^{ff})N(R^{ff})₂, —NR^{ff}C(═NR^{ff})N(R^{ff})₂, —NR^{ee}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, —C₁₋₆ alkyl, —C₁₋₆ haloalkyl, —C₂₋₆ alkenyl, —C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from C₁₋₆ alkyl, —C₁₋₆ haloalkyl, —C₂₋₆ alkenyl, —C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, —C₁₋₆ alkyl, —C₁₋₆ haloalkyl, —C₂₋₆ alkenyl, —C₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, —C(═S)NH(C₁₋₆ alkyl), —C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, —C₁₋₆ alkyl, —C₁₋₆ haloalkyl, —C₂₋₆ alkenyl, —C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

A “counterion” or “anionic counterion” is a negatively charged group associated with a cationic quaternary amino group in order to maintain electronic neutrality. Exemplary counterions include halide ions (e.g., F—, Cl—, Br, I—), NO₃—, ClO₄—, OH—, H₂PO₄—, HSO₄, SO₄⁻² sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quaternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NRC)OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, —C₁₋₁₀ alkyl, —C₁₋₁₀ haloalkyl, —C₂₋₁₀ alkenyl, —C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

These and other exemplary substituents are described in more detail in the Detailed Description, Examples, and Claims. The invention is not intended to be limited in any manner by the above exemplary listing of substituents.

### Other Definitions

As used herein, “pharmaceutical composition” or “pharmaceutical formulation” refer to the combination of a therapeutically active agent with a pharmaceutically acceptable excipient, inert or active, making the composition especially suitable for diagnostic or therapeutic use in vivo or ex vivo.

“Pharmaceutically acceptable” refers to compounds, molecular entities, compositions, materials and/or dosage forms that do not produce an adverse, allergic or other untoward reaction when administered to an animal, or human, as appropriate; or means approved or approvable by a regulatory agency of the federal or a state government or the corresponding agency in countries other than the United States, or that is listed in the U.S. Pharmacopoeia or other generally recognized pharmacopoeia for use in animals, and more particularly, in humans.

As used herein, “pharmaceutically acceptable salt” refers to any salt of an acidic or a basic group that may be present in a compound of the present disclosure (e.g., the compound of Formula A, Formula I or Formula I-1), which salt is compatible with pharmaceutical administration.

As is known to those of skill in the art, “salts” of compounds may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic and benzenesulfonic acid. Other acids, such as oxalic, while not in themselves pharmaceutically acceptable, may be employed in the preparation of salts useful as intermediates in obtaining the compounds described herein and their pharmaceutically acceptable acid addition salts.

Examples of bases include, but are not limited to, alkali metal (e.g., sodium and potassium) hydroxides, alkaline earth metal (e.g., magnesium and calcium) hydroxides, ammonia, and compounds of formula NW₄⁺, wherein W is C1-4 alkyl, and the like.

Examples of salts include, but are not limited, to acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present disclosure compounded with a suitable cation such as Na+, K+, Ca2+, NH4+, and NW4+ (where W can be a C1-4 alkyl group), and the like.

For therapeutic use, salts of the compounds of the present disclosure are contemplated as being pharmaceutically acceptable. However, salts of acids and bases that are non-pharmaceutically acceptable may also find use, for example, in the preparation or purification of a pharmaceutically acceptable compound.

As used herein, “pharmaceutically acceptable excipient” refers to a substance that aids the administration of an active agent to and/or absorption by a subject and can be included in the compositions of the present disclosure without causing a significant adverse toxicological effect on the patient. Non-limiting examples of pharmaceutically acceptable excipients include binders, diluents, carriers, adjuvants, fillers (e.g., brittle diluents or fillers and ductile diluents or fillers), disintegrants, lubricants, coatings, sweeteners, flavors, gelatins, carbohydrates such as lactose, amylose or starch, fatty acid esters, hydroxypropylmethylcellulose, polyvinyl pyrrolidine, and colors, and the like. For examples of excipients, see Gennaro, Remington's Pharmaceutical Sciences, 18th Ed., Mack Publ. Co., Easton, PA (1990) or Shesky, Hancock, Moss and Goldfarb, Handbook of Pharmaceutical Excipients, 9th Ed. Pharmaceutical Press, London, UK (2020).

Examples of diluents or fillers include, but are not limited to, a sugar (e.g., mannitol, lactose, sorbitol, lactitol, erythritol, sucrose, fructose, glucose, agarose, maltose, isomalt, polydextrose, and combinations thereof), an inorganic material (e.g., dibasic calcium phosphate, hydroxyapatite, sodium carbonate, sodium bicarbonate, calcium carbonate, calcium sulfate, magnesium carbonate, magnesium oxide, bentonite, kaolin), calcium lactate, a starch (e.g., a pregelatinized starch), a microcrystalline cellulose, a silicified microcrystalline cellulose, a polysaccharide, a cellulose (e.g., a hydroxypropylcellulose, a hypromellose, a carboxymethylcellulose, a methylcellulose, a hydroxypropylmethylcellulose, a hydroxyethylcellulose), a dextrin, a maltodextrin, an alginate, a collagen, a polyvinylpyrrolidone, a polyvinylacrylate, polyethylene oxide, and polyethylene glycol. Sugar is defined herein to include sugar alcohols.

Examples of disintegrants include, but are not limited to, alginic acid, an alginate, primogel, a cellulose (e.g., hydroxypropylcellulose), polacrillin potassium, sodium starch glycolate, sodium croscarmellose, a polyplasdone (e.g., a crospovidone), and a starch (e.g., corn starch, pregelatinized starch, hydroxypropyl starch, and carboxymethyl starch).

Examples of binders include, but are not limited to, a hydroxypropylcellulose, hydroxyethylcellulose, a hydroxypropylmethycellulose (e.g., a low viscosity hydroxypropylmethycellulose), a sugar, a polyvinylpyrrolidone, a polyvinyl alcohol, a polyvinyl acetate, a polydextrose, a chitosan, a carrageenan, carbophil, a microcrystalline cellulose, gum tragacanth, guar gum, gellan gum, gelatin, and a starch (e.g., corn starch).

Examples of wetting agents include, but are not limited to, a poloxamer (e.g., poloxamer 407), sodium dodecyl sulfate, sodium lauryl sulfate (SLS), sodium stearyl fumarate (SSF), a polydimethylsiloxane, a polysorbate (e.g., polyoxyethylene 20 sorbitan mono-oleate (Tween® 20)), sorbitan monooleate, sorbitan trioleate, sorbitan laurate, sorbitan stearate, sorbitan monopalmitate, lecithin, sodium taurocholate, ursodeoxycholate, polyethoxylated castor oil, cetyl trimethylammonium bromide, nonoxynol, \acute{α}-tocopherol polyethylene glycol 1000 succinate, and docusate sodium.

Examples of lubricants and glidants include, but are not limited to, a wax, a glyceride, a light mineral oil, a polyethylene glycol, sodium stearyl fumarate, magnesium stearate, stearic acid, hydrogenated oil (e.g., hydrogenated vegetable oil), an alkyl sulfate, sodium benzoate, sodium acetate, glyceryl behenate, palmitic acid, and coconut oil. Examples of glidants include, but are not limited to, colloidal silicon dioxide, colloidal silicon dioxide, talc, kaolin, bentonite, and activated carbon/charcoal.

Examples of colorants include, but are not limited to, titanium dioxide, aluminum lakes, iron oxides and carbon black.

Examples of coatings include but are not limited to, a film forming polymer (e.g., a hypromellose, a methyl cellulose, an ethylcellulose, cellulose acetate, a hydroxypropylmethyl cellulose, a hydroxypropyl cellulose, hydroxypropylmethyl cellulose acetate succinate, cellulose acetate phthalate, a polyvinylpyrrolidone, polyvinyl alcohol, a Eudragit/acrylate) and a plasticizer (e.g., triacetin, polyethylene glycol, propylene glycol).

Pharmaceutical compositions for oral administration (e.g., pharmaceutical compositions of the compound of Formula A, Formula I or Formula I-1 described herein) can take the form of bulk liquid solutions or suspensions or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include pills, tablets, capsules or the like in the case of solid compositions.

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

As used herein, “solid dosage form” means a pharmaceutical dose(s) in solid form, e.g., tablets, capsules, granules, powders, minitabs, sachets, stickpacks, reconstitutable powders, dry powder inhalers, lozenges, and chewables.

As used herein, “administering” means oral administration, administration as a pulmonary, suppository, intramuscular administration, intrathecal administration, intranasal administration or subcutaneous administration, or the implantation of a slow-release device, e.g., a mini-osmotic pump, to a subject. Administration is by any route, including transmucosal (e.g., buccal, sublingual, palatal, gingival, nasal, vaginal, rectal, or). Parenteral administration includes, e.g., intramuscular and subcutaneous. Other modes of delivery include, but are not limited to, the use of liposomal formulations, etc. By “co-administer” it is meant that a composition described herein is administered at the same time, just prior to, or just after the administration of one or more additional therapies (e.g., anti-cancer agent, chemotherapeutic, or treatment for a neurodegenerative disease). The compound of Formula A, Formula I or Formula I-1 can be administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation).

The terms “disease,” “disorder,” and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”). In an embodiment, the compounds provided herein are contemplated to be used in methods of therapeutic treatment wherein the action occurs while a subject is suffering from the specified disease, disorder or condition and results in a reduction in the severity of the disease, disorder or condition, or retardation or slowing of the progression of the disease, disorder or condition. In an alternate embodiment, the compounds provided herein are contemplated to be used in methods of prophylactic treatment wherein the action occurs before a subject begins to suffer from the specified disease, disorder or condition and results in preventing a disease, disorder or condition, or one or more symptoms associated with the disease, disorder or condition, or preventing the recurrence of the disease, disorder or condition.

In general, the “effective amount” of a compound refers to an amount sufficient to elicit the desired biological response e.g., to treat a disease or disorder described herein. As will be appreciated by those of ordinary skill in this art, the effective amount of a compound of the disclosure may vary depending on such factors as the desired biological endpoint, the pharmacokinetics of the compound, the disease being treated, the mode of administration, and the age, health, and condition of the subject. An effective amount encompasses therapeutic and prophylactic treatment (i.e., encompasses a “therapeutically effective amount” and a “prophylactically effective amount”).

As used herein, and unless otherwise specified, a “therapeutically effective amount” of a compound is an amount sufficient to provide a therapeutic benefit in the therapeutic treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the therapeutic treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

As used herein, the term “selective” refers to a compound that is at least about 3-fold more potent (e.g., 3-fold, 5-fold, 10-fold, 20-fold, 30-fold, 50-fold, 100-fold, 500-fold, 1000 fold) against one target compared to other targets. For example, a CDK2 degrader that is selective over CCNE (CCNE1 and/or CCNE2) is at least 3-fold more potent (e.g., 3-fold, 5-fold, 10-fold, 20-fold, 30-fold, 50-fold, 100-fold, 500-fold, 1000 fold) more potent against CDK2 than against CCNE (CCNE1 and/or CCNE2). For example, a CCNE (CCNE1 and/or CCNE2) degrader that is selective over CDK2 is at least 3-fold potent (e.g., 3-fold, 5-fold, 10-fold, 20-fold, 30-fold, 50-fold, 100-fold, 500-fold, 1000 fold) more potent against CCNE (CCNE1 and/or CCNE2) than against CDK2. The difference in potency can be determined, for example, by comparing the DC₅₀ values against different targets.

### Compounds

Provided herein are compounds of Formula A, Formula I or Formula I-1. Unless the context requires otherwise, reference throughout this specification to “a compound as described herein,” “a compound of Formula A, Formula I and/or Formula I-1” or “compounds of Formula A, Formula I and/or Formula I-1” refers to all embodiments of Formula A, Formula I and Formula I-1 including, for example, compounds of Formulae A, B, C, D, E, F, G, H, J, K, L, M, N, O, I-A, I-B, I-C, I-1-A, I-1-B, II, II-A, II-B, II-C, II-1, II-1-A, II-1-B, III, III-A, III-B, III-C, III-D, III-E, III-F, III-G, III-1, III-1-A, III-1-B, III-1-C, III-1-D, III-1-E, IV, IV-A, IV-B, IV-C, IV-1, IV-1-A, IV-1-B, V, V-A, V-B, V-C, V-D, V-E, V-F, V-G, V-1, V-1-A, V-1-B, V-1-C, V-1-D, V-1-E, as well as the compounds of Table 1. In an embodiment, provided are compounds of Formula A, Formula I and Formula I-1 or pharmaceutically acceptable salts thereof. In an embodiment, the compounds of Formula A, Formula I and Formula I-1 are provided as pharmaceutically acceptable salts. In an embodiment, the compounds of Formula A, Formula I and Formula I-1 are provided as the corresponding free base (i.e., are not salts).

Included herein, when chemically relevant, are all stereoisomers of the compounds, including diastereomers and enantiomers. Also included are mixtures of possible stereoisomers in any ratio, including, but not limited to, racemic mixtures. Unless stereochemistry is explicitly indicated in a structure, the structure is intended to embrace all possible stereoisomers of the compound depicted. If stereochemistry is explicitly indicated for one portion or portions of a molecule, but not for another portion or portions of a molecule, the structure is intended to embrace all possible stereoisomers for the portion or portions where stereochemistry is not explicitly indicated.

In an embodiment, provided is a compound of Formula A:

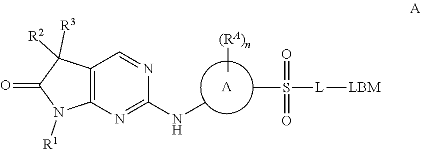

or a pharmaceutically acceptable salt thereof, wherein:
Ring A is phenyl, 3-14 membered cycloalkyl or 4-14 membered heterocyclyl containing 1-3 heteroatoms independently selected from N, O and S;
R¹ is selected from —C₁₋₆ alkyl, —C₂₋₆ alkenyl, —C₂₋₆ alkynyl, —C₁₋₆ haloalkyl, —C₁₋₆ heteroalkyl, —C₃₋₁₄ cycloalkyl, 6-14 membered aryl, 4-14 membered heterocyclyl, 5-14 membered heteroaryl, —C₁₋₄ alkyl-C₃₋₁₄ cycloalkyl, —C₁₋₄ alkyl-(6-14 membered aryl), —C₁₋₄ alkyl-(4-14 membered heterocyclyl), and —C₁₋₄ alkyl (5-14 membered heteroaryl), wherein each heterocyclyl and heteroaryl contains 1-3 heteroatoms independently selected from N, O and S, and wherein each alkyl, alkenyl, alkynyl, cycloalkyl, aryl, heterocyclyl, and heteroaryl is substituted with 0, 1, 2, 3 or 4 instances of R⁶;
each R² and R³ is independently selected from —C₁₋₆ alkyl, —C₁₋₆ haloalkyl, —C₁₋₆ heteroalkyl, —C₂₋₆ alkenyl, —C₂₋₆ alkynyl, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, and wherein each alkyl, alkenyl, alkynyl, cycloalkyl, aryl, heterocyclyl, and heteroaryl is substituted with 0, 1, 2, 3 or 4 instances of R^{B};
or R² and R³, together with the carbon atom to which they are attached, form Ring B, wherein Ring B is a 3-7 membered cycloalkyl ring or a 4-7 membered heterocyclyl ring containing 1 or 3 heteroatoms independently selected from N, O and S, wherein Ring B is substituted with 0, 1, 2, 3, or 4 instances of R^{B};
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(═O)—, —C(═O)O—, —C(═O)—, —S(═O)—, —S(═O)₂—, —NRS(═O)₂—, —S(═O)₂NR—, —NRC(═O)—, —C(═O)NR—, —OC(═O)NR— or —NRC(═O)O—, wherein:
each -Cy- is independently a bivalent ring selected from phenylene, an 8-10 membered bicyclic arylene, a 4-7 membered monocyclic carbocyclylene, a 5-11 membered spiro carbocyclylene, a 4-10 membered bicyclic carbocyclylene, a 5-10 membered bridged carbocyclylene, a 4-7 membered monocyclic heterocyclylene having 1-2 heteroatoms independently selected from nitrogen, oxygen, and sulfur, a 5-11 membered spiro heterocyclylene having 1-2 heteroatoms independently selected from nitrogen, oxygen, and sulfur, an 4-10 membered bicyclic heterocyclylene having 1-2 heteroatoms independently selected from nitrogen, oxygen, and sulfur, a 5-10 membered bridged bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur, wherein each phenylene, arylene, carbocyclylene, heterocyclylene and heteroarylene is substituted with 0, 1, 2, 3, or 4 instances of R^{C};
LBM is selected from

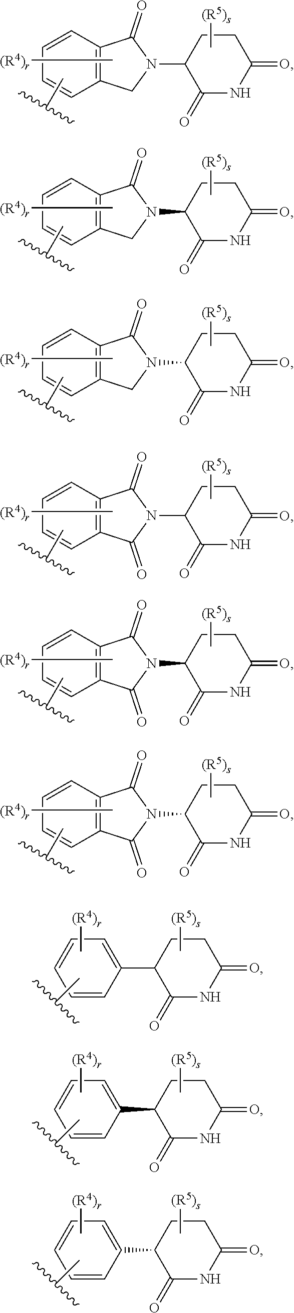

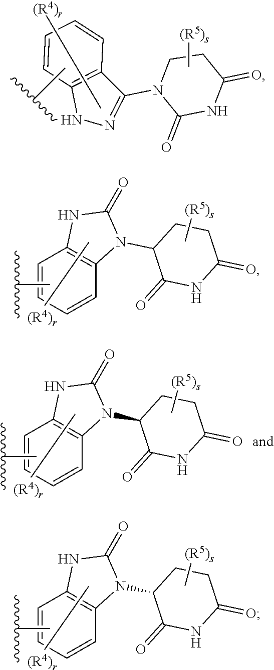

each instance of R^{A}, R^{B}, R^{C}, R⁴, R⁵ and R⁶ is independently selected from oxo, deuterium, halogen, —C₁₋₆ alkyl, —C₁₋₆ heteroalkyl, —C₁₋₆ haloalkyl, —C₂₋₆ alkenyl, —C₂₋₆ alkynyl, —CN, —NO₂, —OR, —SR, —NR₂, —S(O)₂R, —S(O)₂NR₂, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S;
each instance of R is independently hydrogen, —C₁₋₆ alkyl, —C₁₋₆ haloalkyl, —C₁₋₆ heteroalkyl, —C₂₋₆ alkenyl, —C₂₋₆ alkynyl, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, or two R groups attached to the same nitrogen are optionally taken together with nitrogen to which they are attached to form an optionally substituted 4-7 heterocyclyl having 0, 1 or 2 additional heteroatoms independently selected from N, O and S;
n is 0, 1, 2, 3, or 4;
r is 0, 1, 2, 3, or 4; and
s is 0, 1, 2, 3, or 4.

In an embodiment, provided is a compound of Formula A:

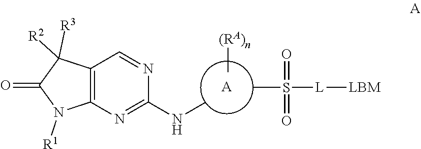

or a pharmaceutically acceptable salt thereof, wherein:
Ring A is 3-14 membered cycloalkyl or 4-14 membered heterocyclyl containing 1-3 heteroatoms independently selected from N, O and S;
R¹ is selected from —C₁₋₆ alkyl, —C₂₋₆ alkenyl, —C₂₋₆ alkynyl, —C₁₋₆ haloalkyl, —C₁₋₆ heteroalkyl, —C₃₋₁₄ cycloalkyl, 6-14 membered aryl, 4-14 membered heterocyclyl, 5-14 membered heteroaryl, —C₁₋₄ alkyl-C₃₋₁₄ cycloalkyl, —C₁₋₄ alkyl-(6-14 membered aryl), —C₁₋₄ alkyl-(4-14 membered heterocyclyl), and —C₁₋₄ alkyl (5-14 membered heteroaryl), wherein each heterocyclyl and heteroaryl contains 1-3 heteroatoms independently selected from N, O and S, and wherein each alkyl, alkenyl, alkynyl, cycloalkyl, aryl, heterocyclyl, and heteroaryl is substituted with 0, 1, 2, 3 or 4 instances of R⁶;
each R² and R³ is independently selected from —C₁₋₆ alkyl, —C₁₋₆ haloalkyl, —C₁₋₆ heteroalkyl, —C₂₋₆ alkenyl, —C₂₋₆ alkynyl, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, and wherein each alkyl, alkenyl, alkynyl, cycloalkyl, aryl, heterocyclyl, and heteroaryl is substituted with 0, 1, 2, 3 or 4 instances of R^{B};
or R² and R³, together with the carbon atom to which they are attached, form Ring B, wherein Ring B is a 3-7 membered cycloalkyl ring or a 4-7 membered heterocyclyl ring containing 1 or 3 heteroatoms independently selected from N, O and S, wherein Ring B is substituted with 0, 1, 2, 3, or 4 instances of R^{B};
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(═O)—, —C(═O)O—, —C(═O)—, —S(═O)—, —S(═O)₂—, —NRS(═O)₂—, —S(═O)₂NR—, —NRC(═O)—, —C(═O)NR—, —OC(═O)NR— or —NRC(═O)O—, wherein:
each -Cy- is independently a bivalent ring selected from phenylene, an 8-10 membered bicyclic arylene, a 4-7 membered monocyclic carbocyclylene, a 5-11 membered spiro carbocyclylene, a 4-10 membered bicyclic carbocyclylene, a 5-10 membered bridged carbocyclylene, a 4-7 membered monocyclic heterocyclylene having 1-2 heteroatoms independently selected from nitrogen, oxygen, and sulfur, a 5-11 membered spiro heterocyclylene having 1-2 heteroatoms independently selected from nitrogen, oxygen, and sulfur, an 4-10 membered bicyclic heterocyclylene having 1-2 heteroatoms independently selected from nitrogen, oxygen, and sulfur, a 5-10 membered bridged bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur, wherein each phenylene, arylene, carbocyclylene, heterocyclylene and heteroarylene is substituted with 0, 1, 2, 3, or 4 instances of R^{C};
LBM is selected from

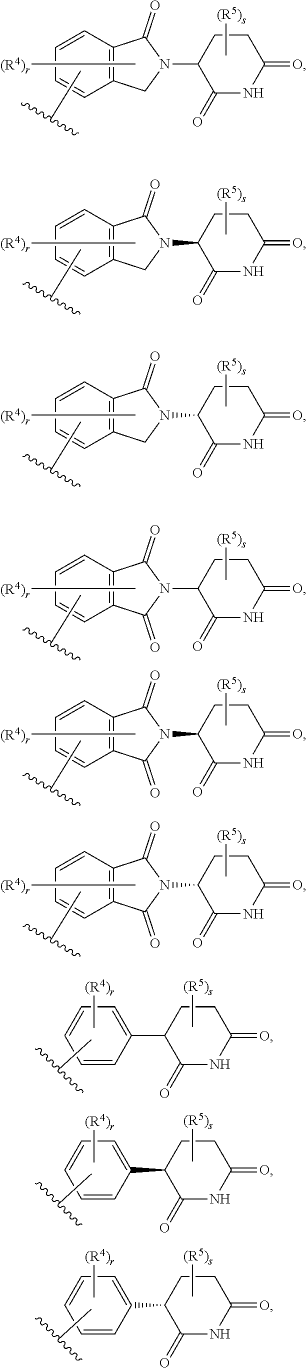

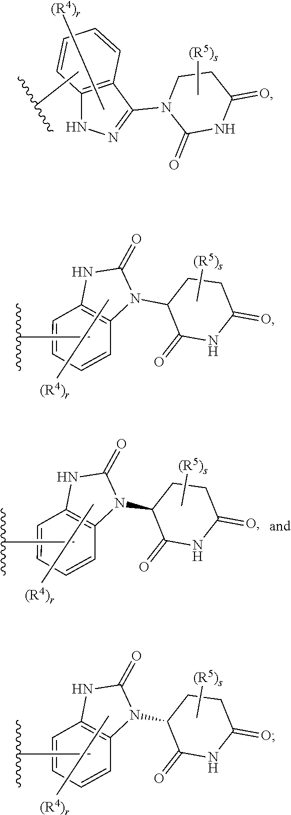

each instance of R^{A}, R^{B}, R^{C}, R⁴, R⁵ and R⁶ is independently selected from oxo, deuterium, halogen, —C₁₋₆ alkyl, —C₁₋₆ heteroalkyl, —C₁₋₆ haloalkyl, —C₂₋₆ alkenyl, —C₂₋₆ alkynyl, —CN, —NO₂, —OR, —SR, —NR₂, —S(O)₂R, —S(O)₂NR₂, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S;
each instance of R is independently hydrogen, —C₁₋₆ alkyl, —C₁₋₆ haloalkyl, —C₁₋₆ heteroalkyl, —C₂₋₆ alkenyl, —C₂₋₆ alkynyl, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, or two R groups attached to the same nitrogen are optionally taken together with nitrogen to which they are attached to form an optionally substituted 4-7 heterocyclyl having 0, 1 or 2 additional heteroatoms independently selected from N, O and S;
n is 0, 1, 2, 3, or 4;
r is 0, 1, 2, 3, or 4; and
s is 0, 1, 2, 3, or 4.

In an embodiment, provided herein is a compound of or Formula I-1:

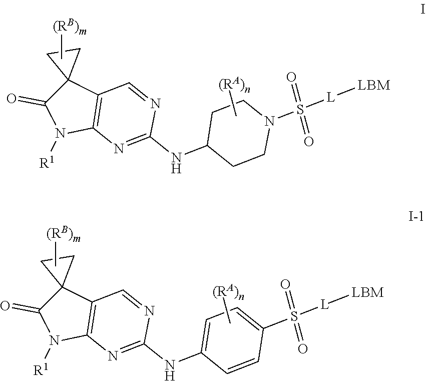

or a pharmaceutically acceptable salt thereof, wherein:
R¹ is selected from —C₁₋₆ alkyl, —C₂₋₆ alkenyl, —C₂₋₆ alkynyl, —C₁₋₆ haloalkyl, —C₁₋₆ heteroalkyl, —C₃₋₁₄ cycloalkyl, 6-14 membered aryl, 4-14 membered heterocyclyl, 5-14 membered heteroaryl, —C₁₋₄ alkyl-C₃₋₁₄ cycloalkyl, —C₁₋₄ alkyl-(6-14 membered aryl), —C₁₋₄ alkyl-(4-14 membered heterocyclyl), and —C₁₋₄ alkyl (5-14 membered heteroaryl), wherein each heterocyclyl and heteroaryl contains 1-3 heteroatoms independently selected from N, O and S, and wherein each alkyl, alkenyl, alkynyl, cycloalkyl, aryl, heterocyclyl, and heteroaryl is substituted with 0, 1, 2, 3 or 4 instances of R⁶;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(═O)—, —C(═O)O—, —C(═O)—, —S(═O)—, —S(═O)₂—, —NRS(═O)₂—, —S(═O)₂NR—, —NRC(═O)—, —C(═O)NR—, —OC(═O)NR— or —NRC(═O)O—, wherein:
each -Cy- is independently a bivalent ring selected from phenylene, an 8-10 membered bicyclic arylene, a 4-7 membered monocyclic carbocyclylene, a 5-11 membered spiro carbocyclylene, a 4-10 membered bicyclic carbocyclylene, a 5-10 membered bridged carbocyclylene, a 4-7 membered monocyclic heterocyclylene having 1-2 heteroatoms independently selected from nitrogen, oxygen, and sulfur, a 5-11 membered spiro heterocyclylene having 1-2 heteroatoms independently selected from nitrogen, oxygen, and sulfur, an 4-10 membered bicyclic heterocyclylene having 1-2 heteroatoms independently selected from nitrogen, oxygen, and sulfur, a 5-10 membered bridged bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur, wherein each phenylene, arylene, carbocyclylene, heterocyclylene and heteroarylene is substituted with 0, 1, 2, 3, or 4 instances of R^{C};
   LBM is selected from

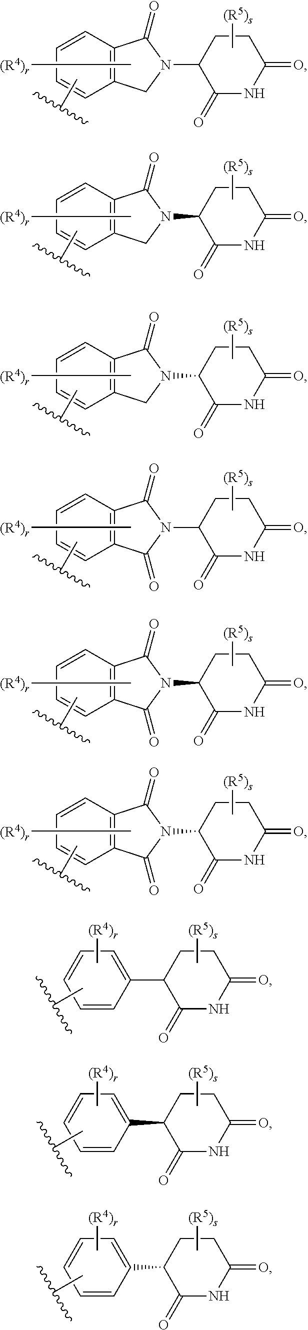

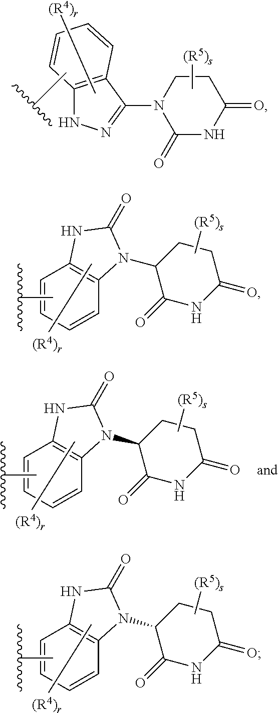

each instance of R^{A}, R^{B}, R^{C}, R⁴, R⁵ and R⁶ is independently selected from oxo, deuterium, halogen, —C₁₋₆ alkyl, —C₁₋₆ heteroalkyl, —C₁₋₆ haloalkyl, —C₂₋₆ alkenyl, —C₂₋₆ alkynyl, —CN, —NO₂, —OR, —SR, —NR₂, —S(O)₂R, —S(O)₂NR₂, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S;
each instance of R is independently hydrogen, —C₁₋₆ alkyl, —C₁₋₆ haloalkyl, —C₁₋₆ heteroalkyl, —C₂₋₆ alkenyl, —C₂₋₆ alkynyl, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, or two R groups attached to the same nitrogen are optionally taken together with nitrogen to which they are attached to form an optionally substituted 4-7 heterocyclyl having 0, 1 or 2 additional heteroatoms independently selected from N, O and S;
m is 0, 1, 2, 3, or 4;
n is 0, 1, 2, 3, or 4;
r is 0, 1, 2, 3, or 4; and
s is 0, 1, 2, 3, or 4.

In an embodiment, provided is a compound of Formula I or Formula I-1:

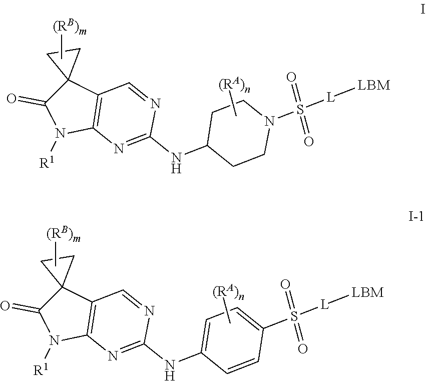

or a pharmaceutically acceptable salt thereof, wherein R¹, R^{A}, L, LBM, m and n are as defined in any of the embodiments described herein.

In an embodiment, the compound is of Formula I.

In an embodiment, the compound is of Formula I-1.

As generally defined herein, Ring B is a 3-7 membered cycloalkyl ring or a 4-7 membered heterocyclyl ring containing 1 or 3 heteroatoms independently selected from N, O and S, wherein Ring B is substituted with 0, 1, 2, 3, or 4 instances of R^{B}, wherein R^{B} is as defined in any of the embodiments described herein. In an embodiment, Ring B is unsubstituted.

In an embodiment, Ring B is a 3-7 membered cycloalkyl ring substituted with 0, 1, 2, 3, or 4 instances of R^{B}, wherein R^{B} is as defined in any of the embodiments described herein. In an embodiment, Ring B is an unsubstituted 3-7 membered cycloalkyl ring. In an embodiment, Ring B is a 3-7 membered monocyclic cycloalkyl ring substituted with 0, 1, 2, 3, or 4 instances of R^{B}, wherein R^{B} is as defined in any of the embodiments described herein. In an embodiment, Ring B is an unsubstituted 3-7 membered monocyclic cycloalkyl ring.

In an embodiment, Ring B is selected from cyclopropyl and cyclobutyl.

In an embodiment, Ring B is selected from cyclopropyl, cyclobutyl, cyclopentyl and cyclohexyl substituted with 0, 1, 2, 3, or 4 instances of R^{B}, wherein R^{B} is as defined in any of the embodiments described herein. In an embodiment, Ring B is selected from cyclopropyl and cyclobutyl substituted with 0, 1, 2, 3, or 4 instances of R^{B}, wherein R^{B} is as defined in any of the embodiments described herein. In an embodiment, Ring B is selected from unsubstituted cyclopropyl, cyclobutyl, cyclopentyl and cyclohexyl. In an embodiment, Ring B is selected from unsubstituted cyclopropyl and cyclobutyl. In an embodiment, Ring B is cyclopropyl substituted with 0, 1, 2, 3, or 4 instances of R^{B}, wherein R^{B} is as defined in any of the embodiments described herein. In an embodiment, Ring B is cyclopropyl substituted with 0, 1 or 2 instances of R^{B}, wherein R^{B} is as defined in any of the embodiments described herein. In an embodiment, Ring B is unsubstituted cyclopropyl.

In an embodiment, Ring B is cyclobutyl substituted with 0, 1, 2, 3, or 4 instances of R^{B}, wherein R^{B} is as defined in any of the embodiments described herein. In an embodiment, Ring B is unsubstituted cyclobutyl. In an embodiment, Ring B is cyclopentyl substituted with 0, 1, 2, 3, or 4 instances of R^{B}, wherein R^{B} is as defined in any of the embodiments described herein. In an embodiment, Ring B is unsubstituted cyclopentyl. In an embodiment, Ring B is cyclohexyl substituted with 0, 1, 2, 3, or 4 instances of R^{B}, wherein R^{B} is as defined in any of the embodiments described herein. In an embodiment, Ring B is unsubstituted cyclohexyl.

In an embodiment, Ring B is a 4-7 membered heterocyclyl ring containing 1 or 3 heteroatoms independently selected from N, O and S (e.g., azetidinyl, oxetanyl, pyrrolidinyl, tetrahydrofuranyl, piperidinyl, morpholinyl, piperazinyl, tetrahydropyranyl), wherein Ring B is substituted with 0, 1, 2, 3, or 4 instances of R^{B}. In an embodiment, Ring B is an unsubstituted 4-7 membered heterocyclyl ring containing 1 or 3 heteroatoms independently selected from N, O and S (e.g., azetidinyl, oxetanyl, pyrrolidinyl, tetrahydrofuranyl, piperidinyl, morpholinyl, piperazinyl, tetrahydropyranyl).

As generally defined herein, Ring A is phenyl, 3-14 membered cycloalkyl or 4-14 membered heterocyclyl containing 1-3 heteroatoms independently selected from N, O and S.

In an embodiment, Ring A is 3-14 membered cycloalkyl or 4-14 membered heterocyclyl containing 1-3 heteroatoms independently selected from N, O and S.

In an embodiment, Ring A is a monocyclic 3-7 membered cycloalkyl (e.g., cyclopropane, cyclobutane, cyclopentane, cyclohexane, cycloheptane). In an embodiment, Ring A is selected from cyclopropane, cyclobutane, cyclopentane, cyclohexane and cycloheptane.

In an embodiment, Ring A is a monocyclic 4-7 membered heterocyclyl containing 1-2 heteroatoms independently selected from N, O and S (including S(O) and S(O)₂). In an embodiment, Ring A is a monocyclic 4-7 membered heterocyclyl containing 1-2 heteroatoms independently selected from N and O. In an embodiment, Ring A is a monocyclic 4-7 membered heterocyclyl containing 1-2 nitrogen atoms. In an embodiment, Ring A is a monocyclic 4-7 membered heterocyclyl containing 1 nitrogen atom. In an embodiment, Ring A is a monocyclic 4-7 membered heterocyclyl containing 2 nitrogen atoms.

In an embodiment, Ring A is selected from azetidine, pyrrolidine, piperidine, azepane, oxetane, tetrahydrofuran, tetrahydropyran, piperazine, morpholine and diazepane.

In an embodiment, Ring A is selected from azetidine, pyrrolidine, piperidine, and azepane.

In an embodiment, Ring A is azetidine. In an embodiment, Ring A is pyrrolidine. In an embodiment, Ring A is piperidine. In an embodiment, Ring A is azepane. In an embodiment, Ring A is piperidin-4-yl. In an embodiment, Ring A is

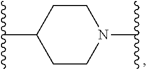 [SMILES: *C1CCN(*)CC1]

wherein the nitrogen atom is attached to the S(═O)₂ group and the 4-position carbon is attached to the NH of the compound of Formula I.

In an embodiment, Ring A is phenyl. In an embodiment, Ring A is

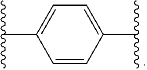 [SMILES: *c1ccc(*)cc1]

As generally defined herein, R¹ is selected from —C₁₋₆ alkyl, —C₂₋₆ alkenyl, —C₂₋₆ alkynyl, —C₁₋₆ haloalkyl, —C₁₋₆ heteroalkyl, —C₃₋₁₄ cycloalkyl, 6-14 membered aryl, 4-14 membered heterocyclyl, 5-14 membered heteroaryl, —C₁₋₄ alkyl-C₃₋₁₄ cycloalkyl, —C₁₋₄ alkyl-(6-14 membered aryl), —C₁₋₄ alkyl-(4-14 membered heterocyclyl), and —C₁₋₄ alkyl (5-14 membered heteroaryl), wherein each heterocyclyl and heteroaryl contains 1-3 heteroatoms independently selected from N, O and S, and wherein each alkyl, alkenyl, alkynyl, cycloalkyl, aryl, heterocyclyl, and heteroaryl is substituted with 0, 1, 2, 3 or 4 instances of R⁶, wherein each R⁶ is as defined herein.

In an embodiment, R¹ is selected from —C₁₋₆ haloalkyl, —C₃₋₇ cycloalkyl, phenyl, 4-7 membered heterocyclyl (e.g., heterocyclyl containing 1 or 2 atoms independently selected from N, O and S, including S(O) and S(O)₂), and 5-6 membered heteroaryl (e.g., heteroaryl containing 1, 2 or 3 heteroatoms independently selected from N, O and S), each of which is substituted with 0, 1, 2, 3 or 4 instances of R⁶, wherein each instance of R⁶ is as defined in any of the embodiments described herein.

In an embodiment, R¹ is selected from C₁₋₆ haloalkyl, C₃₋₇ cycloalkyl, and phenyl, each of which is substituted with 0, 1, 2, 3 or 4 instances of R⁶, wherein each instance of R⁶ is as defined in any of the embodiments described herein.

In an embodiment, R¹ is selected from —C₃₋₇ cycloalkyl, phenyl and —C₁₋₄ alkyl-C₃₋₁₄ cycloalkyl, each of which is substituted with 0, 1, 2, 3 or 4 instances of R⁶, wherein each instance of R⁶ is as defined in any of the embodiments described herein.

In an embodiment, R¹ is cyclopentyl, cyclohexyl, phenyl or —CH(CH₃)-cyclopropyl, each substituted with 0, 1, 2, 3 or 4 instances of R⁶, wherein each instance of R⁶ is as defined in any of the embodiments described herein.

In an embodiment, R¹ is cyclopentyl, cyclohexyl, phenyl or —CH(CH₃)-cyclopropyl, each substituted with 0, 1 or 2 instances of R⁶, wherein each instance of R⁶ is as defined in any of the embodiments described herein.

In an embodiment, R¹ is cyclopentyl or cyclohexyl, each substituted with 0, 1, 2, 3 or 4 instances of R⁶, wherein each instance of R⁶ is as defined in any of the embodiments described herein.

In an embodiment, R¹ is C₃₋₇ cycloalkyl substituted with 0, 1, 2, 3 or 4 instances of R⁶ wherein each instance of R⁶ is as defined in any of the embodiments described herein.

In an embodiment, R¹ is monocyclic C₃₋₇ cycloalkyl substituted with 0, 1, 2, 3 or 4 instances of R⁶ wherein each instance of R⁶ is as defined in any of the embodiments described herein.

In an embodiment, R¹ is cyclopropyl, cyclobutyl, cyclopentyl, cyclohexyl or cycloheptyl substituted with 0, 1, 2, 3 or 4 instances of R⁶ wherein each instance of R⁶ is as defined in any of the embodiments described herein.

In an embodiment, R¹ is cyclopentyl substituted with 0, 1, 2, 3 or 4 instances of R⁶ wherein each instance of R⁶ is as defined in any of the embodiments described herein.

In an embodiment, R¹ is cyclohexyl substituted with 0, 1, 2, 3 or 4 instances of R⁶ wherein each instance of R⁶ is as defined in any of the embodiments described herein. In an embodiment, R¹ is selected from 1,1-difluorobutan-2-yl, cyclopentyl, 2-methylcyclopentyl, 3-hydroxycyclohexyl, 2-hydroxy-2-methylcyclopentyl, 2-methylphenyl, 2-chloro-5-fluorophenyl 1,5-dimethyl-1H-pyrazol-4-yl, 7-chloro-1,2,3,4-tetrahydroisoquinolin-6-yl, and 1-cyclopropylethyl.

In an embodiment, R¹ is selected from cyclopentyl, 2-methylcyclopentyl, 3-hydroxycyclohexyl, 2-methylphenyl, and 1,1-difluorobutan-2-yl.

In an embodiment, R¹ is selected from cyclopentyl, 2-methylcyclopentyl, 2-methylphenyl, and 1,1-difluorobutan-2-yl. In an embodiment, R¹ is selected from 2-methylcyclopentyl and 3-hydroxycyclohexyl.

In an embodiment, R¹ is selected from

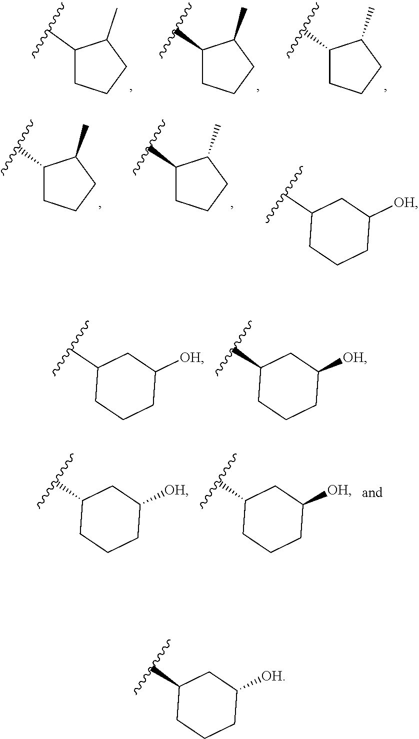

In an embodiment, R¹ is selected from:

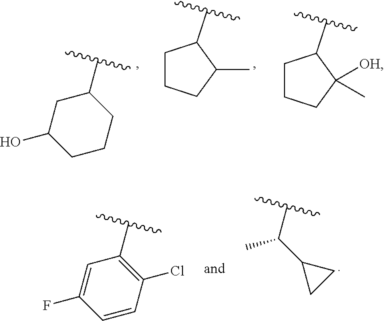

In an embodiment, R¹ is selected from:

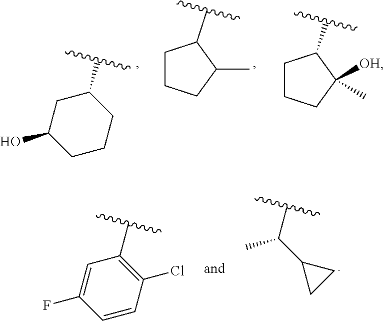

In an embodiment, R¹ is selected from:

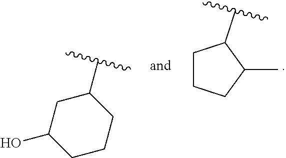

In an embodiment, R¹ is selected from:

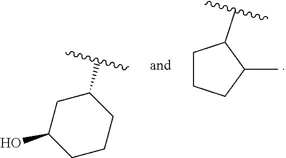

In an embodiment, R¹ is 2-methylcyclopentyl. In an embodiment, R¹ is selected from

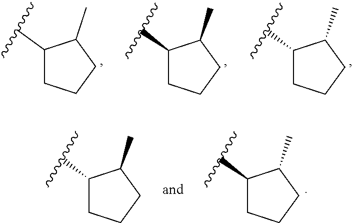

In an embodiment, R¹ is

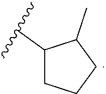 [SMILES: *C1CCCC1C]

In an embodiment, R¹ is

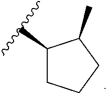 [SMILES: *[C@@H]1CCC[C@@H]1C]

In an embodiment, R¹ is

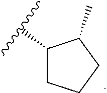 [SMILES: *[C@H]1CCC[C@H]1C]

In an embodiment, R¹ is

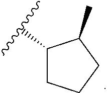 [SMILES: *[C@H]1CCC[C@@H]1C]

In an embodiment, R¹ is

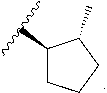 [SMILES: *[C@@H]1CCC[C@H]1C]

In an embodiment, R¹ is 3-hydroxycyclohexyl. In an embodiment, R¹ is selected from

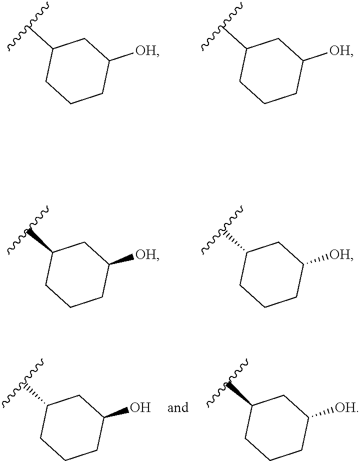 [SMILES: *C1CCCC(O)C1 ; *C1CCCC(O)C1 ; *[C@@H]1CCC[C@H](O)C1 ; *[C@H]1CCC[C@@H](O)C1 ; *[C@H]1CCC[C@H](O)C1 ; *[C@@H]1CCC[C@@H](O)C1]

In an embodiment, R¹ is

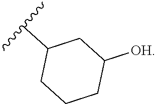 [SMILES: *C1CCCC(O)C1]

In an embodiment, R¹ is

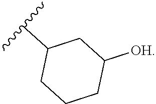 [SMILES: *C1CCCC(O)C1]

In an embodiment, R¹ is

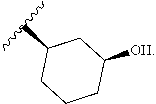 [SMILES: *[C@@H]1CCC[C@H](O)C1]

In an embodiment, R¹ is

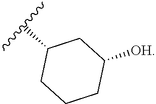 [SMILES: *[C@H]1CCC[C@@H](O)C1]

In an embodiment, R¹ is

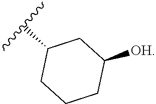 [SMILES: *[C@H]1CCC[C@H](O)C1]

In an embodiment, R¹ is

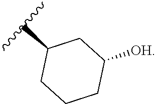 [SMILES: *[C@@H]1CCC[C@@H](O)C1]

In an embodiment, R¹ is —C₁₋₆ haloalkyl (e.g., —CH₂CF₃, CH₂CHF₂, CH₂CH₂F).

In an embodiment, R¹ is phenyl, substituted with 0, 1, 2, 3 or 4 instances of R⁶, wherein each instance of R⁶ is as defined in any of the embodiments described herein.

In an embodiment, R¹ is 4-7 membered heterocyclyl containing 1 or 2 atoms independently selected from N, O and S, including S(O) and S(O)₂ (e.g., oxetanyl, azetidinyl, tetrahydrofuranyl, pyrolidinyl, tetrahydropyranyl, tetrahydrothiopyranyl, morpholinyl, piperidinyl, piperazinyl, azepanyl), each of which is substituted with 0, 1, 2, 3 or 4 instances of R⁶, wherein each instance of R⁶ is as defined in any of the embodiments described herein.

In an embodiment, R¹ is 5-6 membered monocyclic heteroaryl containing 1, 2 or 3 heteroatoms independently selected from N, O and S (e.g., furanyl, pyrrolyl, thiophenyl, thiazolyl, oxazolyl, isothiazolyl, isoxazolyl, pyrazolyl, imidazolyl, triazolyl, thiadiazolyl, oxadiazolyl, pyridinyl, pyrimidinyl, pyrazinyl, diazinyl, triazinyl), each of which is substituted with 0, 1, 2, 3 or 4 instances of R⁶, wherein each instance of R⁶ is as defined in any of the embodiments described herein.

As generally defined herein, each R² and R³ is independently selected from —C₁₋₆ alkyl, —C₁₋₆ haloalkyl, —C₁₋₆ heteroalkyl, —C₂₋₆ alkenyl, —C₂₋₆ alkynyl, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, and wherein each alkyl, alkenyl, alkynyl, cycloalkyl, aryl, heterocyclyl, and heteroaryl is substituted with 0, 1, 2, 3 or 4 instances of R^{B}, wherein each R^{B} is as defined herein.

In an embodiment, each R² and R³ is independently —C₁₋₆ alkyl. In an embodiment, each R² and R³ is independently selected from -Me, -Et-, —Pr, —ⁱPr and ^{t}Bu. In an embodiment, each R² and R³ is -Me.

In an embodiment, R² and R³, together with the carbon atom to which they are attached, form Ring B.

As generally defined herein, L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(═O)—, —C(═O)O—, —C(═O)—, —S(═O)—, —S(═O)₂—, —NRS(═O)₂—, —S(═O)₂NR—, —NRC(═O)—, —C(═O)NR—, —OC(═O)NR— or —NRC(═O)O—, wherein each -Cy- and —R are as defined in any of the embodiments described herein.

In an embodiment, L is selected from:

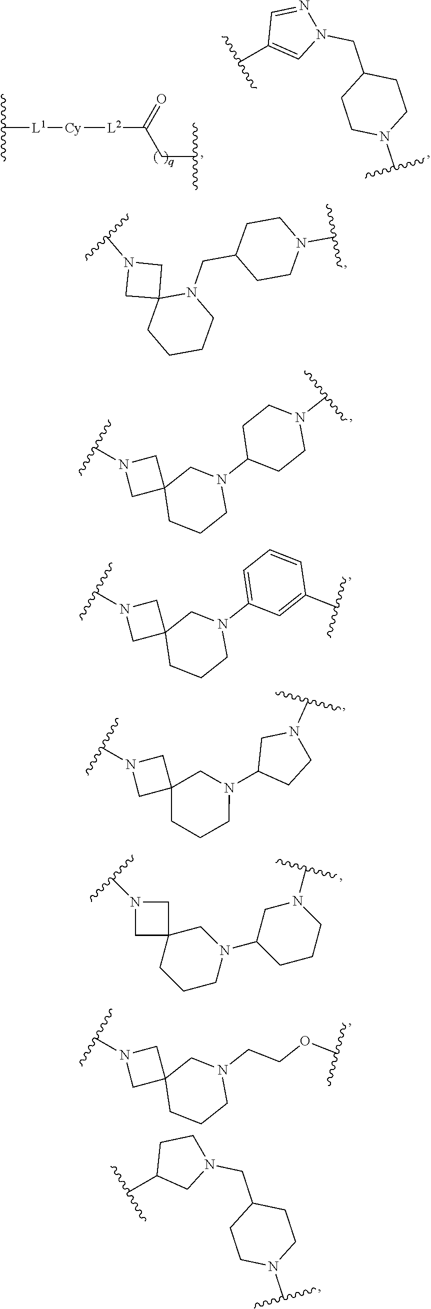

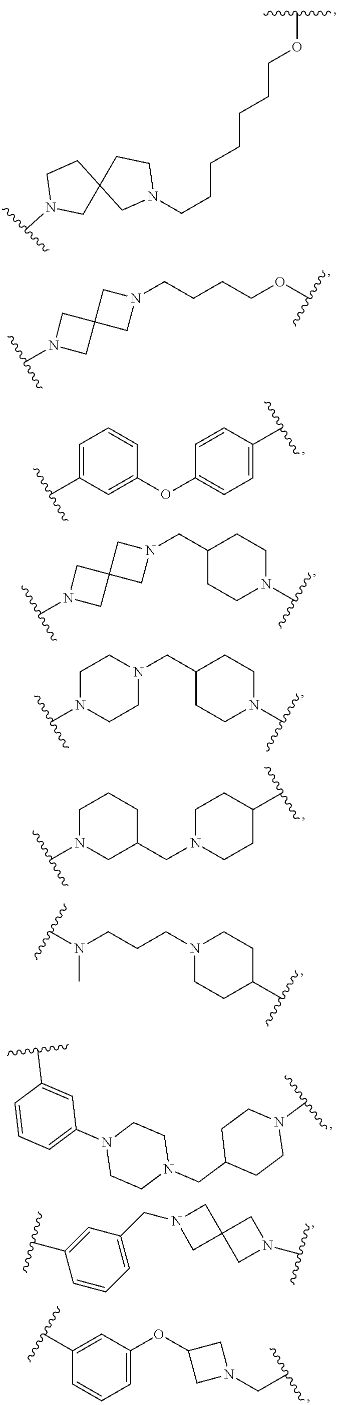

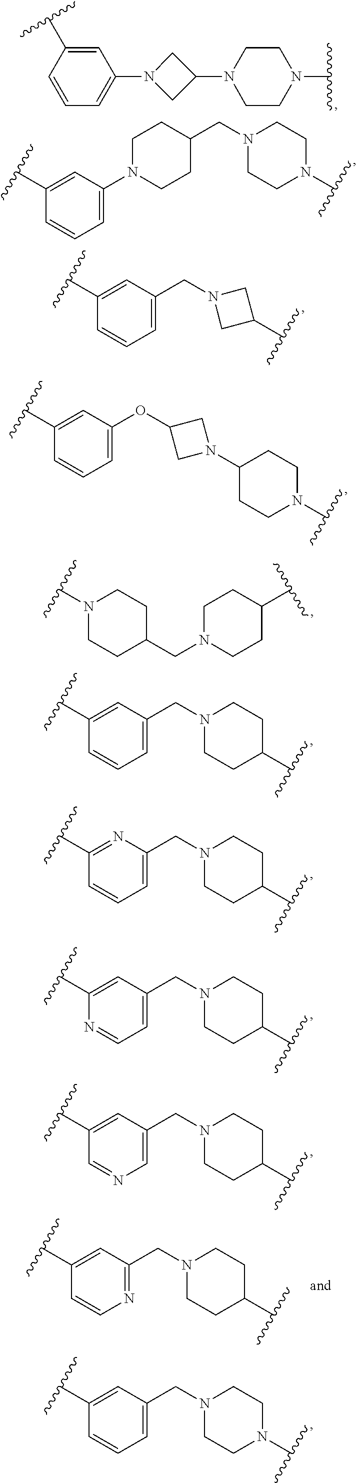

and each substituted with 0, 1, 2 or 3 instances of R⁷, wherein each R⁷ is independently as defined in any of the embodiments described herein;
wherein the left side attachment point connects to the —S(O)₂— group and the right side attachment point connects to LBM;
L¹ and L² are each independently selected from a bond and —N(R′), wherein R′ is selected from H and C₁₋₆ alkyl; and
q is 0, 1, 2, 3, 4, 5, 6, 7, 8, 9 or 10.

In an embodiment, L Is selected from:

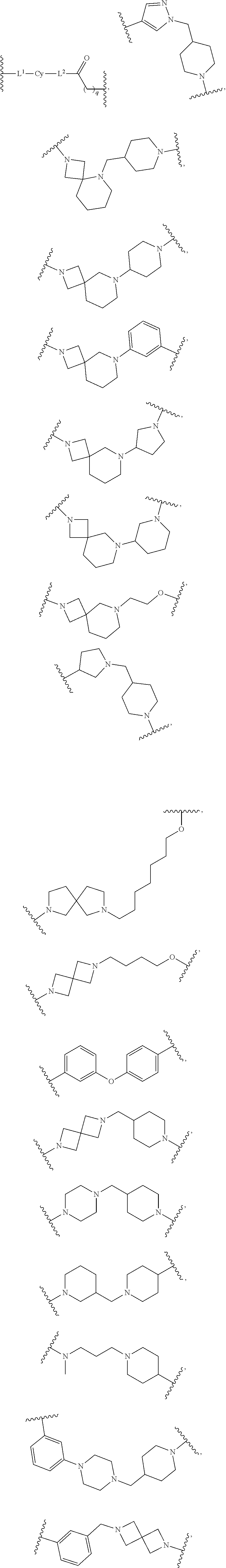

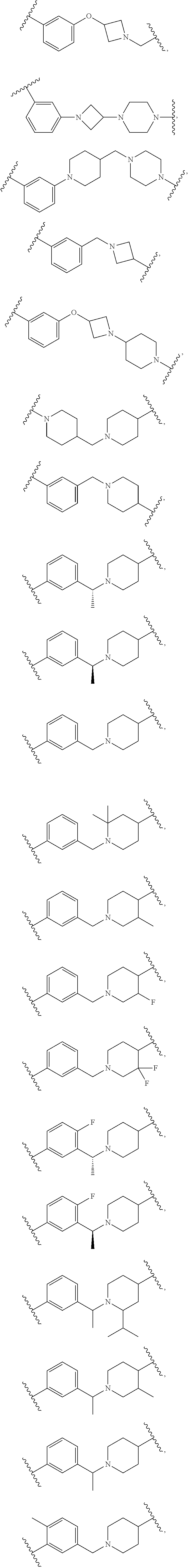

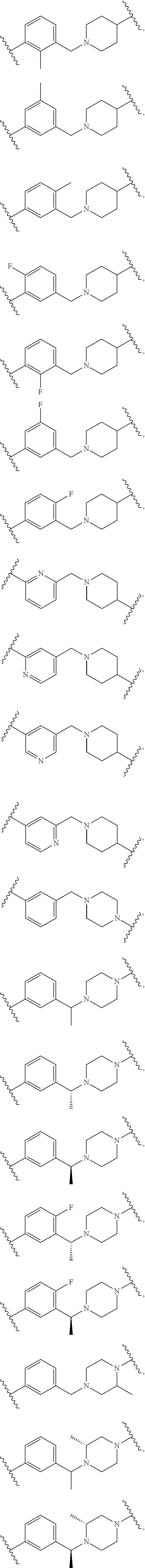

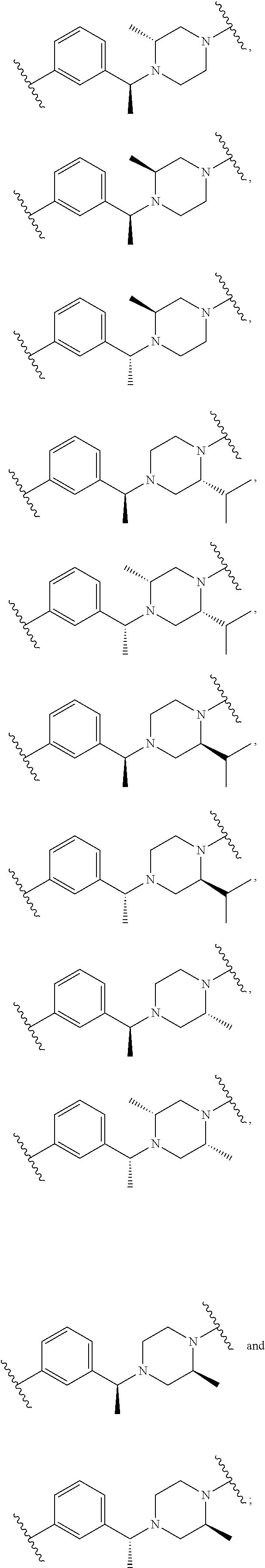

wherein the left side attachment point connects to the —S(O)₂— group and the right side attachment point connects to LBM;
L¹ and L² are each independently selected from a bond and —N(R′), wherein R′ is selected from H and C₁₋₆ alkyl; and
q is 0, 1, 2, 3, 4, 5, 6, 7, 8, 9 or 10.

In an embodiment, L is selected from:

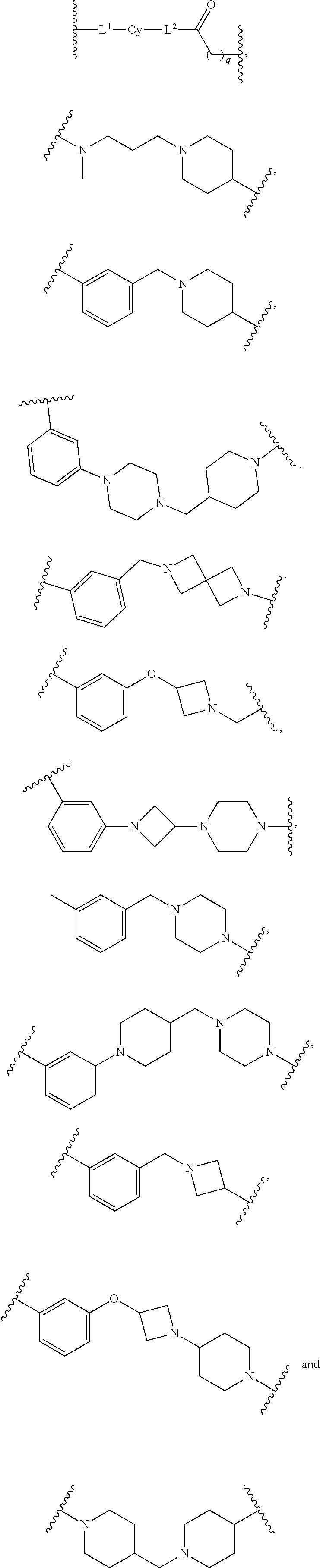

wherein the left side attachment point connects to the —S(O)₂— group and the right side attachment point connects to LBM and L¹, L², -Cy- and q are as defined in any of the embodiments described herein.

In an embodiment, L is selected from:

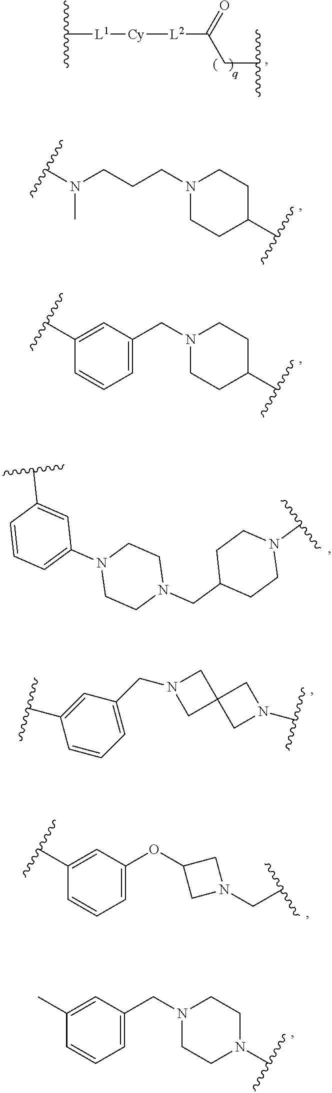

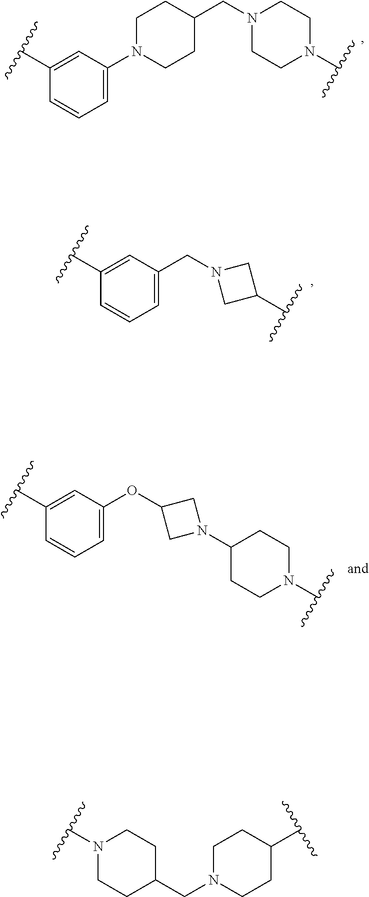 [SMILES: *c1cccc(N2CCC(CN3CCN(*)CC3)CC2)c1 ; *c1cccc(CN2CC(*)C2)c1 ; *c1cccc(OC2CN(C3CCN(*)CC3)C2)c1 ; *C1CCN(CC2CCN(*)CC2)CC1]

wherein the left side attachment point connects to the —S(O)₂— group and the right side attachment point connects to LBM and L¹, L², -Cy- and q are as defined in any of the embodiments described herein.

In an embodiment, L is selected from:

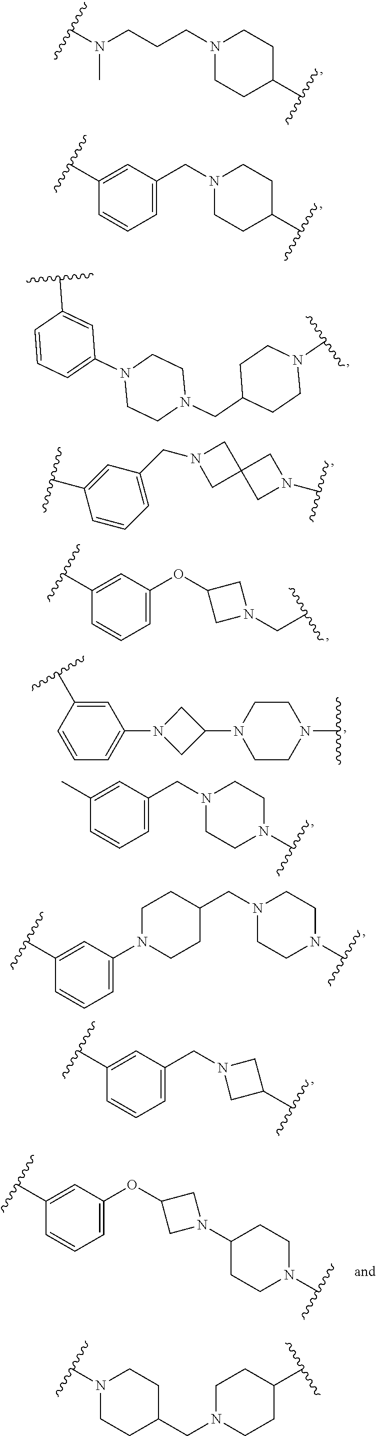

wherein the left side attachment point connects to the —S(O)₂— group and the right side attachment point connects to LBM.

In an embodiment, L is selected from:

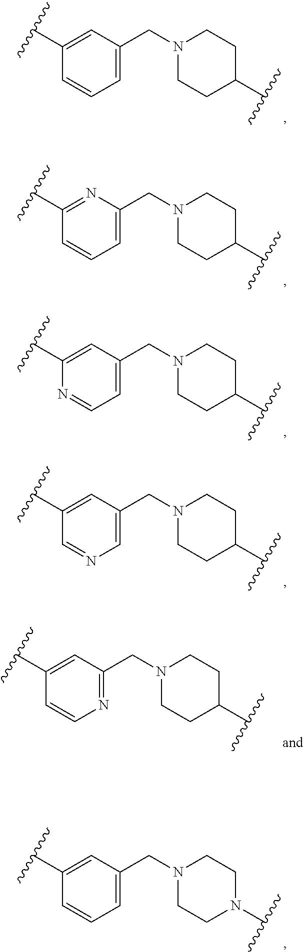 [SMILES: *c1cccc(CN2CCC(*)CC2)c1 ; *c1cccc(CN2CCC(*)CC2)n1 ; *c1cc(CN2CCC(*)CC2)ccn1 ; *c1cncc(CN2CCC(*)CC2)c1 ; *c1ccnc(CN2CCC(*)CC2)c1 ; *c1cccc(CN2CCN(*)CC2)c1]

and, each substituted with 0, 1, 2 or 3 instances of R⁷, wherein R⁷ is as defined in any of the embodiments described herein; wherein the left side attachment point connects to the —S(O)₂— group and the right side attachment point connects to LBM.

In an embodiment, L is

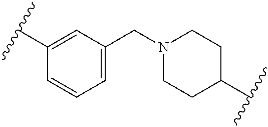 [SMILES: *c1cccc(CN2CCC(*)CC2)c1]

substituted with 0, 1, 2 or 3 instances of R⁷, wherein R⁷ is as defined in any of the embodiments described herein; wherein the left side attachment point connects to the —S(O)₂— group and the right side attachment point connects to LBM. In an embodiment, L is

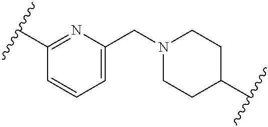 [SMILES: *c1cccc(CN2CCC(*)CC2)n1]

substituted with 0, 1, 2 or 3 instances of R⁷, wherein R⁷ is as defined in any of the embodiments described herein; wherein the left side attachment point connects to the —S(O)₂-group and the right side attachment point connects to LBM. In an embodiment, L is

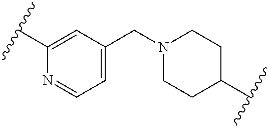 [SMILES: *c1cc(CN2CCC(*)CC2)ccn1]

substituted 0, 1, 2 or 3 instances of R⁷, wherein R⁷ is as defined in any of the embodiments described herein; wherein the left side attachment point connects to the —S(O)₂— group and the right side attachment point connects to LBM. In an embodiment, L is

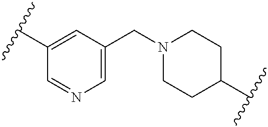 [SMILES: *c1cncc(CN2CCC(*)CC2)c1]

substituted with 0, 1, 2 or 3 instances of R⁷, wherein R⁷ is as defined in any of the embodiments described herein; wherein the left side attachment point connects to the —S(O)₂— group and the right side attachment point connects to LBM. In an embodiment, L is

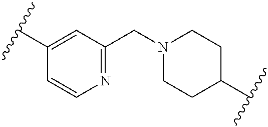 [SMILES: *c1ccnc(CN2CCC(*)CC2)c1]

substituted with 0, 1, 2 or 3 instances of R⁷, wherein R⁷ is as defined in any of the embodiments described herein; wherein the left side attachment point connects to the —S(O)₂— group and the right side attachment point connects to LBM. In an embodiment, L is

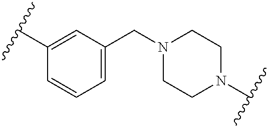 [SMILES: *c1cccc(CN2CCN(*)CC2)c1]

substituted with 0, 1, 2 or 3 instances of R⁷, wherein R⁷ is as defined in any of the embodiments described herein; wherein the left side attachment point connects to the —S(O)₂— group and the right side attachment point connects to LBM. In an embodiment, L is

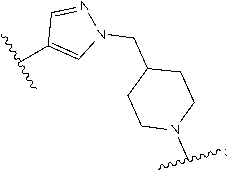 [SMILES: *c1cnn(CC2CCN(*)CC2)c1]

wherein the left side attachment point connects to the —S(O)₂— group and the right side attachment point connects to LBM. In an embodiment, L is

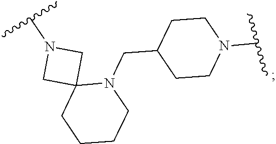 [SMILES: *N1CCC(CN2CCCCC23CN(*)C3)CC1]

wherein the left side attachment point connects to the —S(O)₂— group and the right side attachment point connects to LBM. In an embodiment, L is

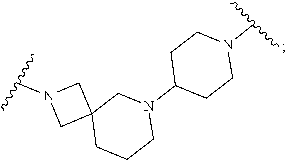 [SMILES: *N1CCC(N2CCCC3(CN(*)C3)C2)CC1]

wherein the left side attachment point connects to the —S(O)₂— group and the right side attachment point connects to LBM. In an embodiment, L is

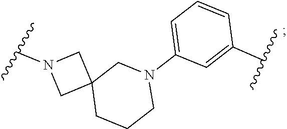 [SMILES: *c1cccc(N2CCCC3(CN(*)C3)C2)c1]

wherein the left side attachment point connects to the —S(O)₂— group and the right side attachment point connects to LBM. In an embodiment, L is

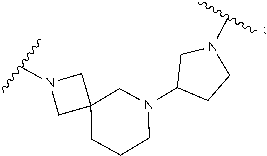 [SMILES: *N1CCC(N2CCCC3(CN(*)C3)C2)C1]

wherein the left side attachment point connects to the —S(O)₂— group and the right side attachment point connects to LBM. In an embodiment, L is

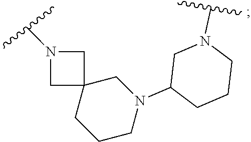 [SMILES: *N1CCCC(N2CCCC3(CN(*)C3)C2)C1]

wherein the left side attachment point connects to the —S(O)₂— group and the right side attachment point connects to LBM. In an embodiment, L is

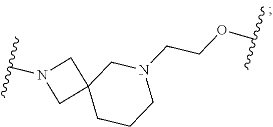 [SMILES: *OCCN1CCCC2(CN(*)C2)C1]

wherein the left side attachment point connects to the —S(O)₂-group and the right side attachment point connects to LBM. In an embodiment, L is

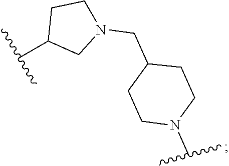 [SMILES: *C1CCN(CC2CCN(*)CC2)C1]

wherein the left side attachment point connects to the —S(O)₂— group and the right side attachment point connects to LBM. In an embodiment, L is

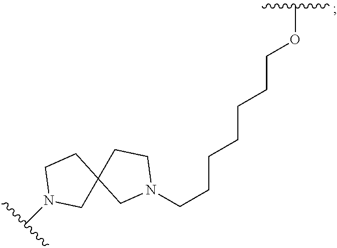 [SMILES: *OCCCCCCCN1CCC2(CCN(*)C2)C1]

wherein the left side attachment point connects to the —S(O)₂-group and the right side attachment point connects to LBM. In an embodiment, L is

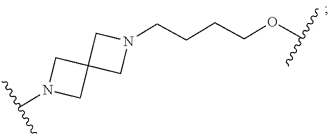 [SMILES: *OCCCCN1CC2(CN(*)C2)C1]

wherein the left side attachment point connects to the —S(O)₂-group and the right side attachment point connects to LBM. In an embodiment, L is

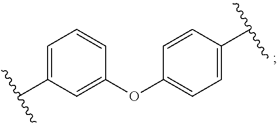 [SMILES: *c1ccc(Oc2cccc(*)c2)cc1]

wherein the left side attachment point connects to the —S(O)₂— group and the right side attachment point connects to LBM. In an embodiment, L is

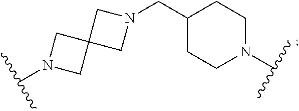 [SMILES: *N1CCC(CN2CC3(CN(*)C3)C2)CC1]

wherein the left side attachment point connects to the —S(O)₂-group and the right side attachment point connects to LBM. In an embodiment, L is

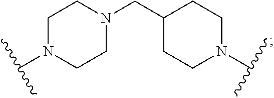 [SMILES: *N1CCC(CN2CCN(*)CC2)CC1]

wherein the left side attachment point connects to the —S(O)₂— group and the right side attachment point connects to LBM. In an embodiment, L is

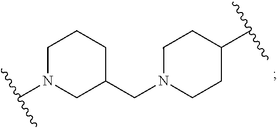 [SMILES: *C1CCN(CC2CCCN(*)C2)CC1]

wherein the left side attachment point connects to the —S(O)₂— group and the right side attachment point connects to LBM. In an embodiment, L is

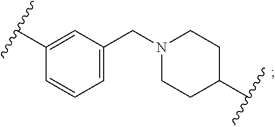 [SMILES: *c1cccc(CN2CCC(*)CC2)c1]

wherein the left side attachment point connects to the —S(O)₂— group and the right side attachment point connects to LBM. In an embodiment, L is

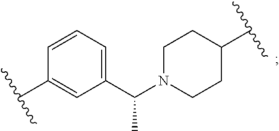 [SMILES: *c1cccc([C@@H](C)N2CCC(*)CC2)c1]

wherein the left side attachment point connects to the —S(O)₂— group and the right side attachment point connects to LBM. The compound of any one of claims 1-115, or a pharmaceutically acceptable salt thereof, wherein L is

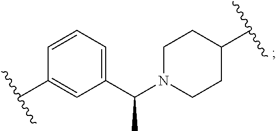 [SMILES: *c1cccc([C@H](C)N2CCC(*)CC2)c1]

wherein the left side attachment point connects to the —S(O)₂— group and the right side attachment point connects to LBM. In an embodiment, L is

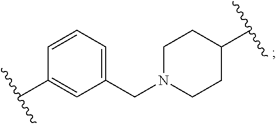 [SMILES: *c1cccc(CN2CCC(*)CC2)c1]

wherein the left side attachment point connects to the —S(O)₂— group and the right side attachment point connects to LBM. In an embodiment, L is

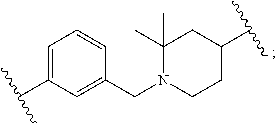 [SMILES: *c1cccc(CN2CCC(*)CC2(C)C)c1]

wherein the left side attachment point connects to the —S(O)₂— group and the right side attachment point connects to LBM. In an embodiment, L is

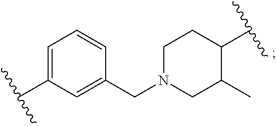 [SMILES: *c1cccc(CN2CCC(*)C(C)C2)c1]

wherein the left side attachment point connects to the —S(O)₂— group and the right side attachment point connects to LBM. In an embodiment, L is

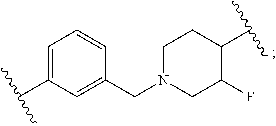 [SMILES: *c1cccc(CN2CCC(*)C(F)C2)c1]

wherein the left side attachment point connects to the —S(O)₂— group and the right side attachment point connects to LBM. In an embodiment, L is

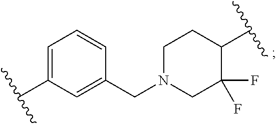 [SMILES: *c1cccc(CN2CCC(*)C(F)(F)C2)c1]

wherein the left side attachment point connects to the —S(O)₂— group and the right side attachment point connects to LBM. In an embodiment, L is

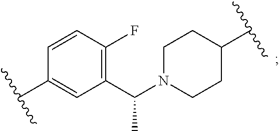 [SMILES: *c1ccc(F)c([C@@H](C)N2CCC(*)CC2)c1]

wherein the left side attachment point connects to the —S(O)₂-group and the right side attachment point connects to LBM. In an embodiment, L is

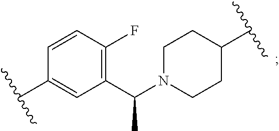 [SMILES: *c1ccc(F)c([C@H](C)N2CCC(*)CC2)c1]

wherein the left side attachment point connects to the —S(O)₂— group and the right side attachment point connects to LBM. In an embodiment, L is

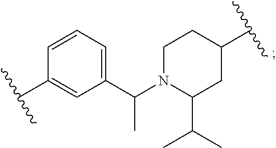 [SMILES: *c1cccc(C(C)N2CCC(*)CC2C(C)C)c1]

wherein the left side attachment point connects to the —S(O)₂— group and the right side attachment point connects to LBM. In an embodiment, L is

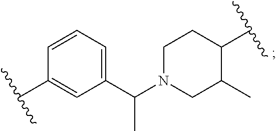 [SMILES: *c1cccc(C(C)N2CCC(*)C(C)C2)c1]

wherein the left side attachment point connects to the —S(O)₂— group and the right side attachment point connects to LBM. In an embodiment, L is

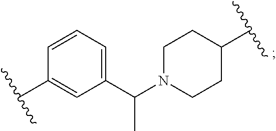 [SMILES: *c1cccc(C(C)N2CCC(*)CC2)c1]

wherein the left side attachment point connects to the —S(O)₂— group and the right side attachment point connects to LBM. In an embodiment, L is

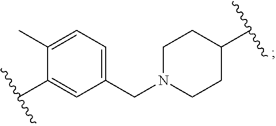 [SMILES: *c1cc(CN2CCC(*)CC2)ccc1C]

wherein the left side attachment point connects to the —S(O)₂— group and the right side attachment point connects to LBM. In an embodiment, L is

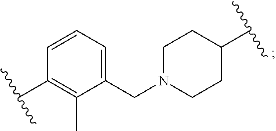 [SMILES: *c1cccc(CN2CCC(*)CC2)c1C]

wherein the left side attachment point connects to the —S(O)₂— group and the right side attachment point connects to LBM. In an embodiment, L is

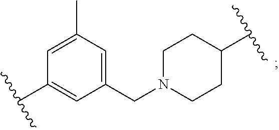 [SMILES: *c1cc(C)cc(CN2CCC(*)CC2)c1]

wherein the left side attachment point connects to the —S(O)₂— group and the right side attachment point connects to LBM. In an embodiment, L is

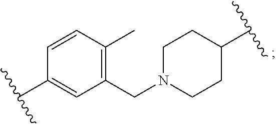 [SMILES: *c1ccc(C)c(CN2CCC(*)CC2)c1]

wherein the left side attachment point connects to the —S(O)₂— group and the right side attachment point connects to LBM. In an embodiment, L is

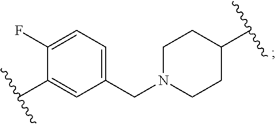 [SMILES: *c1cc(CN2CCC(*)CC2)ccc1F]

wherein the left side attachment point connects to the —S(O)₂— group and the right side attachment point connects to LBM. In an embodiment, L is

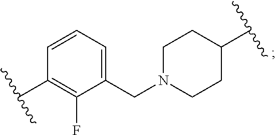 [SMILES: *c1cccc(CN2CCC(*)CC2)c1F]

wherein the left side attachment point connects to the —S(O)₂— group and the right side attachment point connects to LBM. In an embodiment, L is

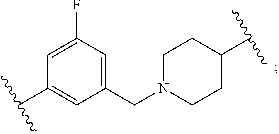 [SMILES: *c1cc(F)cc(CN2CCC(*)CC2)c1]

wherein the left side attachment point connects to the —S(O)₂— group and the right side attachment point connects to LBM. In an embodiment, L is

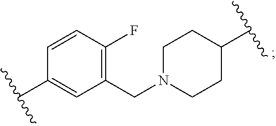 [SMILES: *c1ccc(F)c(CN2CCC(*)CC2)c1]

wherein the left side attachment point connects to the —S(O)₂— group and the right side attachment point connects to LBM. In an embodiment, L is

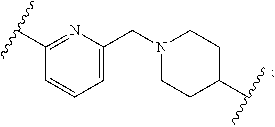 [SMILES: *c1cccc(CN2CCC(*)CC2)n1]

wherein the left side attachment point connects to the —S(O)₂— group and the right side attachment point connects to LBM. In an embodiment, L is

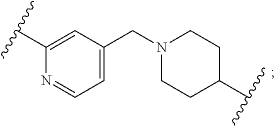 [SMILES: *c1cc(CN2CCC(*)CC2)ccn1]

wherein the left side attachment point connects to the —S(O)₂— group and the right side attachment point connects to LBM. In an embodiment, L is

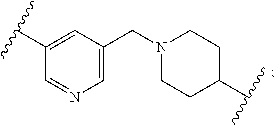 [SMILES: *c1cncc(CN2CCC(*)CC2)c1]

wherein the left side attachment point connects to the —S(O)₂— group and the right side attachment point connects to LBM. In an embodiment, L is

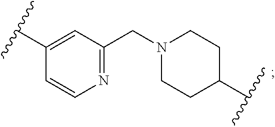 [SMILES: *c1ccnc(CN2CCC(*)CC2)c1]

wherein the left side attachment point connects to the —S(O)₂— group and the right side attachment point connects to LBM. In an embodiment, L is

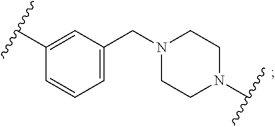 [SMILES: *c1cccc(CN2CCN(*)CC2)c1]

wherein the left side attachment point connects to the —S(O)₂— group and the right side attachment point connects to LBM. In an embodiment, L is

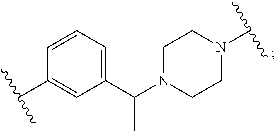 [SMILES: *c1cccc(C(C)N2CCN(*)CC2)c1]

wherein the left side attachment point connects to the —S(O)₂— group and the right side attachment point connects to LBM. In an embodiment, L is

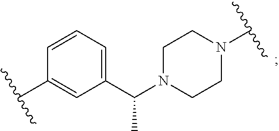 [SMILES: *c1cccc([C@@H](C)N2CCN(*)CC2)c1]

wherein the left side attachment point connects to the —S(O)₂— group and the right side attachment point connects to LBM. In an embodiment, L is

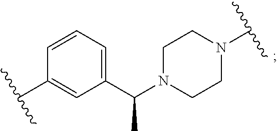 [SMILES: *c1cccc([C@H](C)N2CCN(*)CC2)c1]

wherein the left side attachment point connects to the —S(O)₂— group and the right side attachment point connects to LBM. In an embodiment, L is

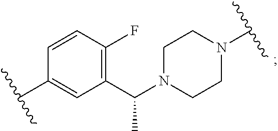 [SMILES: *c1ccc(F)c([C@@H](C)N2CCN(*)CC2)c1]

wherein the left side attachment point connects to the —S(O)₂— group and the right side attachment point connects to LBM. In an embodiment, L is

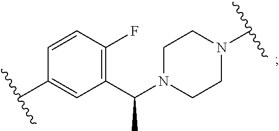 [SMILES: *c1ccc(F)c([C@H](C)N2CCN(*)CC2)c1]

wherein the left side attachment point connects to the —S(O)₂— group and the right side attachment point connects to LBM. In an embodiment, L is

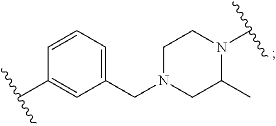 [SMILES: *c1cccc(CN2CCN(*)C(C)C2)c1]

wherein the left side attachment point connects to the —S(O)₂— group and the right side attachment point connects to LBM. In an embodiment, L is

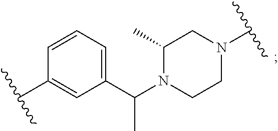 [SMILES: *c1cccc(C(C)N2CCN(*)C[C@H]2C)c1]

wherein the left side attachment point connects to the —S(O)₂— group and the right side attachment point connects to LBM. In an embodiment, L is

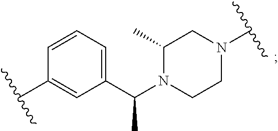 [SMILES: *c1cccc([C@H](C)N2CCN(*)C[C@H]2C)c1]

wherein the left side attachment point connects to the —S(O)₂— group and the right side attachment point connects to LBM. In an embodiment, L is

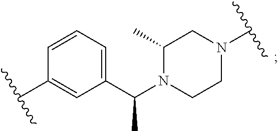 [SMILES: *c1cccc([C@H](C)N2CCN(*)C[C@H]2C)c1]

wherein the left side attachment point connects to the —S(O)₂— group and the right side attachment point connects to LBM. In an embodiment, L is

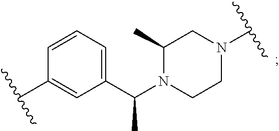 [SMILES: *c1cccc([C@H](C)N2CCN(*)C[C@@H]2C)c1]

wherein the left side attachment point connects to the —S(O)₂— group and the right side attachment point connects to LBM. In an embodiment, L is

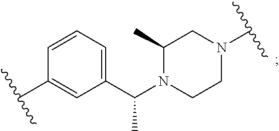 [SMILES: *c1cccc([C@@H](C)N2CCN(*)C[C@@H]2C)c1]

wherein the left side attachment point connects to the —S(O)₂— group and the right side attachment point connects to LBM. In an embodiment, L is

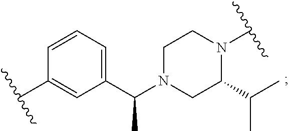 [SMILES: *c1cccc([C@H](C)N2CCN(*)[C@H](C(C)C)C2)c1]

wherein the left side attachment point connects to the —S(O)₂-group and the right side attachment point connects to LBM. In an embodiment, L is

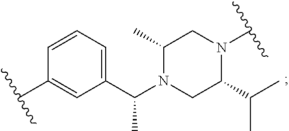 [SMILES: *c1cccc([C@@H](C)N2C[C@@H](C(C)C)N(*)C[C@H]2C)c1]

wherein the left side attachment point connects to the —S(O)₂-group and the right side attachment point connects to LBM. In an embodiment, L is

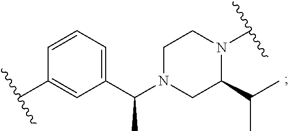 [SMILES: *c1cccc([C@H](C)N2CCN(*)[C@@H](C(C)C)C2)c1]

wherein the left side attachment point connects to the —S(O)₂-group and the right side attachment point connects to LBM. In an embodiment, L is

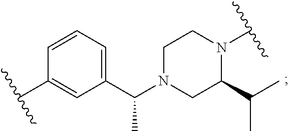 [SMILES: *c1cccc([C@@H](C)N2CCN(*)[C@@H](C(C)C)C2)c1]

wherein the left side attachment point connects to the —S(O)₂-group and the right side attachment point connects to LBM. In an embodiment, L is

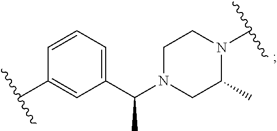 [SMILES: *c1cccc([C@H](C)N2CCN(*)[C@H](C)C2)c1]

wherein the left side attachment point connects to the —S(O)₂— group and the right side attachment point connects to LBM. In an embodiment, L is

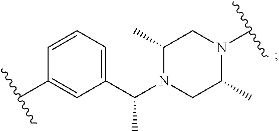 [SMILES: *c1cccc([C@@H](C)N2C[C@@H](C)N(*)C[C@H]2C)c1]

wherein the left side attachment point connects to the —S(O)₂— group and the right side attachment point connects to LBM. In an embodiment, L is

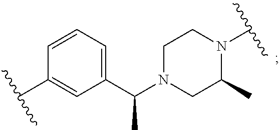 [SMILES: *c1cccc([C@H](C)N2CCN(*)[C@@H](C)C2)c1]

wherein the left side attachment point connects to the —S(O)₂— group and the right side attachment point connects to LBM. In an embodiment, L is

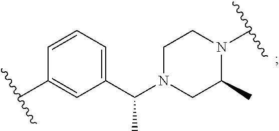 [SMILES: *c1cccc([C@@H](C)N2CCN(*)[C@@H](C)C2)c1]

wherein the left side attachment point connects to the —S(O)₂— group and the right side attachment point connects to LBM. In an embodiment, L is

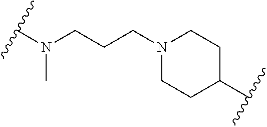 [SMILES: *C1CCN(CCCN(*)C)CC1]

wherein the left side attachment point connects to the —S(O)₂— group and the right side attachment point connects to LBM. In an embodiment, L is

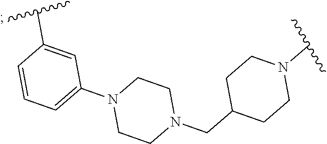 [SMILES: *c1cccc(N2CCN(CC3CCN(*)CC3)CC2)c1]

N wherein the left side attachment point connects to the —S(O)₂-group and the right side attachment point connects to LBM. In an embodiment, L is

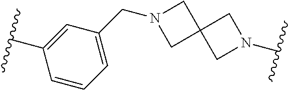 [SMILES: *c1cccc(CN2CC3(CN(*)C3)C2)c1]

wherein the left side attachment point connects to the —S(O)₂-group and the right side attachment point connects to LBM. In an embodiment, L is

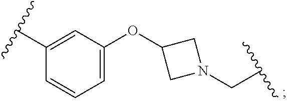 [SMILES: *CN1CC(Oc2cccc(*)c2)C1]

wherein the left side attachment point connects to the —S(O)₂— group and the right side attachment point connects to LBM. In an embodiment, L is

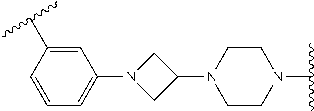 [SMILES: *c1cccc(N2CC(N3CCN(*)CC3)C2)c1]

wherein the left side attachment point connects to the —S(O)₂-group and the right side attachment point connects to LBM. In an embodiment, L is

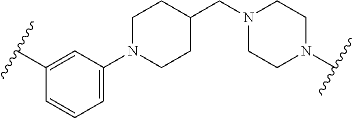 [SMILES: *c1cccc(N2CCC(CN3CCN(*)CC3)CC2)c1]

wherein the left side attachment point connects to the —S(O)₂-group and the right side attachment point connects to LBM. In an embodiment, L is

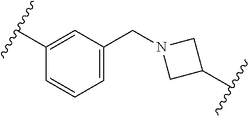 [SMILES: *c1cccc(CN2CC(*)C2)c1]

wherein the left side attachment point connects to the —S(O)₂— group and the right side attachment point connects to LBM. In an embodiment, L is

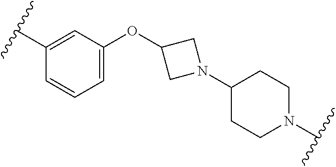 [SMILES: *c1cccc(OC2CN(C3CCN(*)CC3)C2)c1]

wherein the left side attachment point connects to the —S(O)₂-group and the right side attachment point connects to LBM. In an embodiment, L is

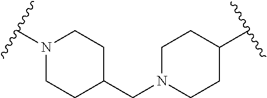 [SMILES: *C1CCN(CC2CCN(*)CC2)CC1]

wherein the left side attachment point connects to the —S(O)₂— group and the right side attachment point connects to LBM.
In an embodiment, L is

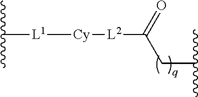

wherein L¹, -Cy-, L² and q are as defined in any of the embodiments described herein and wherein the left side attachment point connects to the —S(O)₂— group and the right side attachment point connects to LBM.

In an embodiment,

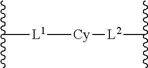

is selected from

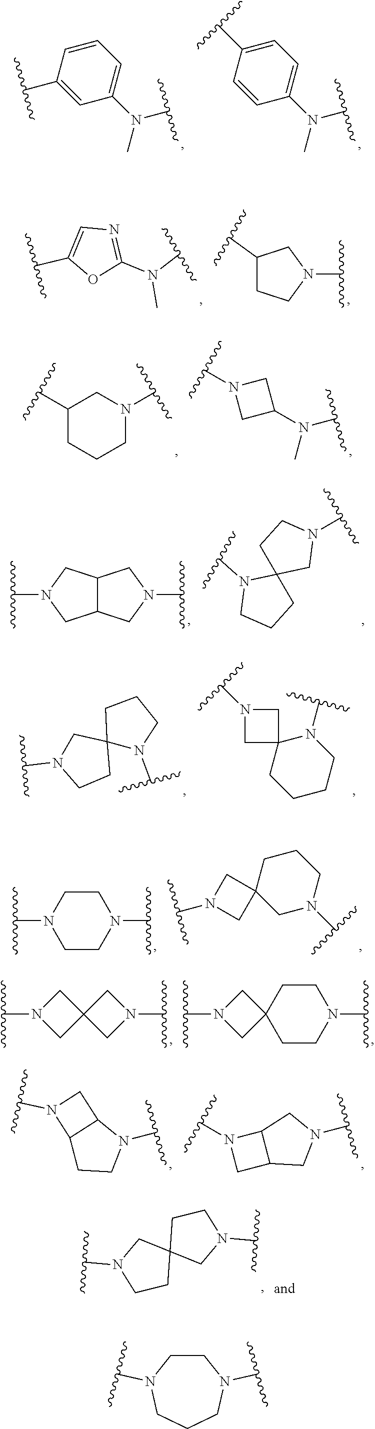

wherein the left attachment point connects to —S(O)₂— and the right attachment point connects to LBM.

In an embodiment,

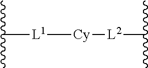

is selected from

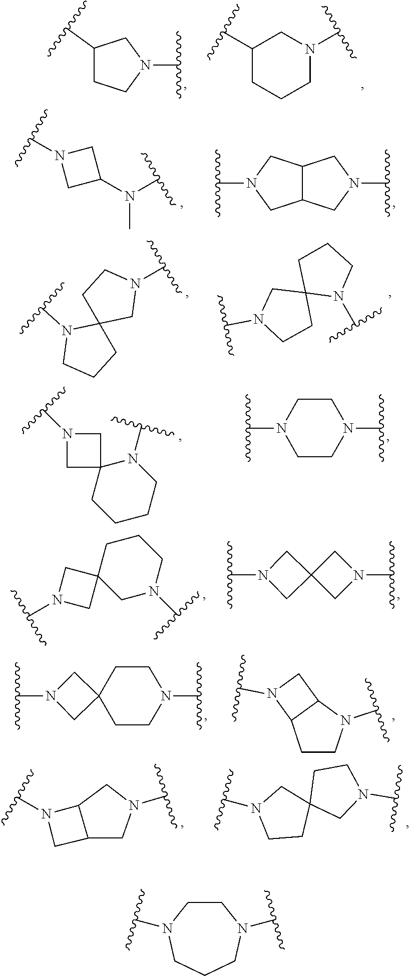

wherein the left attachment point connects to —S(O)₂— and the right attachment point connects to LBM.

In an embodiment,

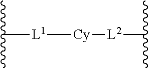

is

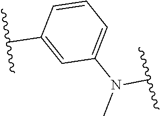 [SMILES: *c1cccc(N(*)C)c1]

wherein the left attachment point connects to —S(O)₂— and the right attachment point connects to LBM. In an embodiment,

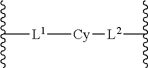

is

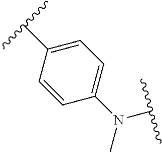 [SMILES: *c1ccc(N(*)C)cc1]

wherein the left attachment point connects to —S(O)₂— and the right attachment point connects to LBM. In an embodiment,

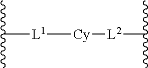

is

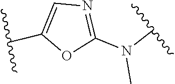 [SMILES: *c1cnc(N(*)C)o1]

wherein the left attachment point connects to —S(O)₂— and the right attachment point connects to LBM. In an embodiment,

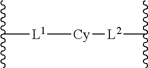

is

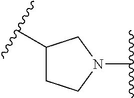 [SMILES: *C1CCN(*)C1]

wherein the left attachment point connects to —S(O)₂— and the right attachment point connects to LBM. In an embodiment,

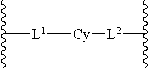

is

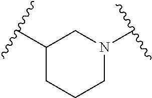 [SMILES: *C1CCCN(*)C1]

wherein the left attachment point connects to —S(O)₂— and the right attachment point connects to LBM. In an embodiment,

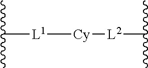

is

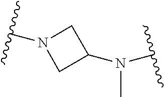 [SMILES: *N1CC(N(*)C)C1]

wherein the left attachment point connects to —S(O)₂— and the right attachment point connects to LBM. In an embodiment,

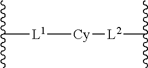

is

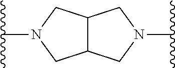 [SMILES: *N1CC2CN(*)CC2C1]

wherein the left attachment point connects to —S(O)₂— and the right attachment point connects to LBM. In an embodiment,

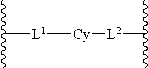

is

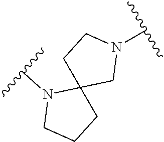 [SMILES: *N1CCC2(CCCN2*)C1]

wherein the left attachment point connects to —S(O)₂— and the right attachment point connects to LBM. In an embodiment,

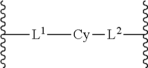

is

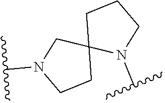 [SMILES: *N1CCC2(CCCN2*)C1]

wherein the left attachment point connects to —S(O)₂— and the right attachment point connects to LBM. In an embodiment,

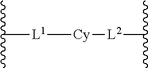

is

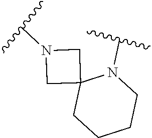 [SMILES: *N1CC2(CCCCN2*)C1]

wherein the left attachment point connects to —S(O)₂— and the right attachment point connects to LBM. In an embodiment,

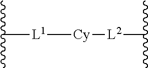

is

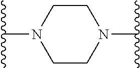 [SMILES: *N1CCN(*)CC1]

wherein the left attachment point connects to —S(O)₂— and the right attachment point connects to LBM. In an embodiment,

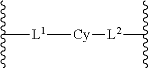

is

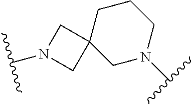 [SMILES: *N1CCCC2(C1)CN(*)C2]

wherein the left attachment point connects to —S(O)₂— and the right attachment point connects to LBM. In an embodiment,

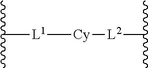

is

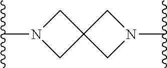 [SMILES: *N1CC2(C1)CN(*)C2]

wherein the left attachment point connects to —S(O)₂— and the right attachment point connects to LBM. In an embodiment,

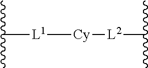

is

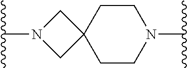 [SMILES: *N1CCC2(CC1)CN(*)C2]

wherein the left attachment point connects to —S(O)₂— and the right attachment point connects to LBM. In an embodiment,

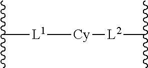

is

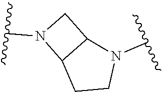 [SMILES: *N1CCC2C1CN2*]

wherein the left attachment point connects to —S(O)₂— and the right attachment point connects to LBM. In an embodiment,

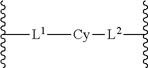

is

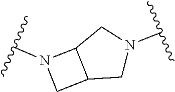 [SMILES: *N1CC2CN(*)C2C1]

wherein the left attachment point connects to —S(O)₂— and the right attachment point connects to LBM. In an embodiment,

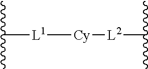

is

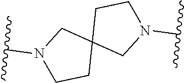 [SMILES: *N1CCC2(CCN(*)C2)C1]

wherein the left attachment point connects to —S(O)₂— and the right attachment point connects to LBM. In an embodiment,

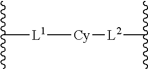

is

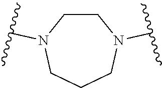 [SMILES: *N1CCCN(*)CC1]

wherein the left attachment point connects to —S(O)₂— and the right attachment point connects to LBM.

In an embodiment, L is selected from

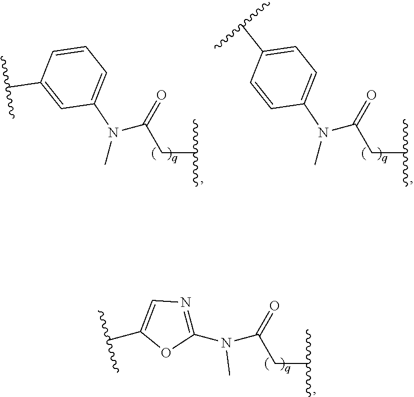

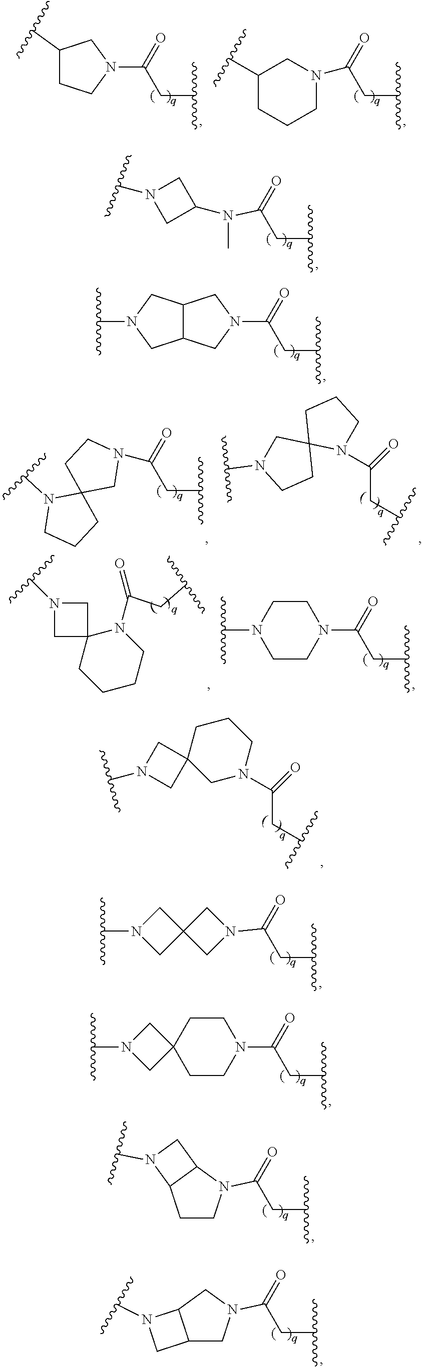

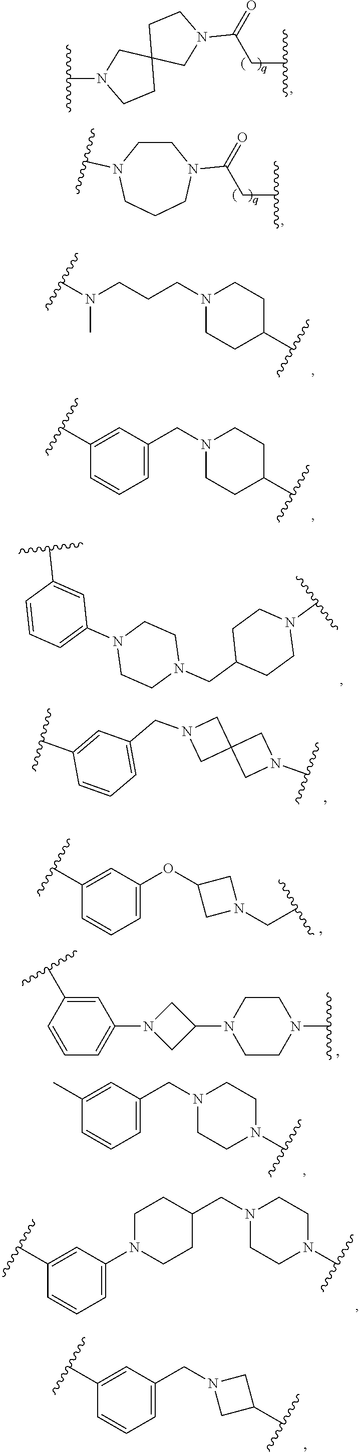

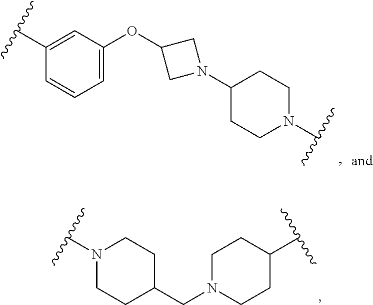

wherein q is as defined in any of the embodiments described herein and wherein the left attachment point connects to —S(O)₂— and the right attachment point connects to LBM.

In an embodiment, L is selected from

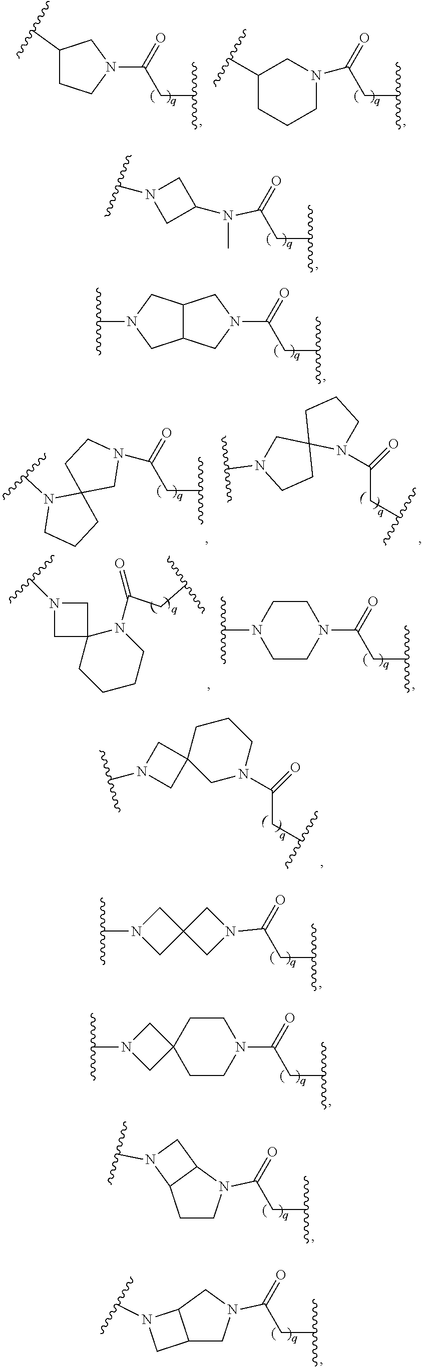

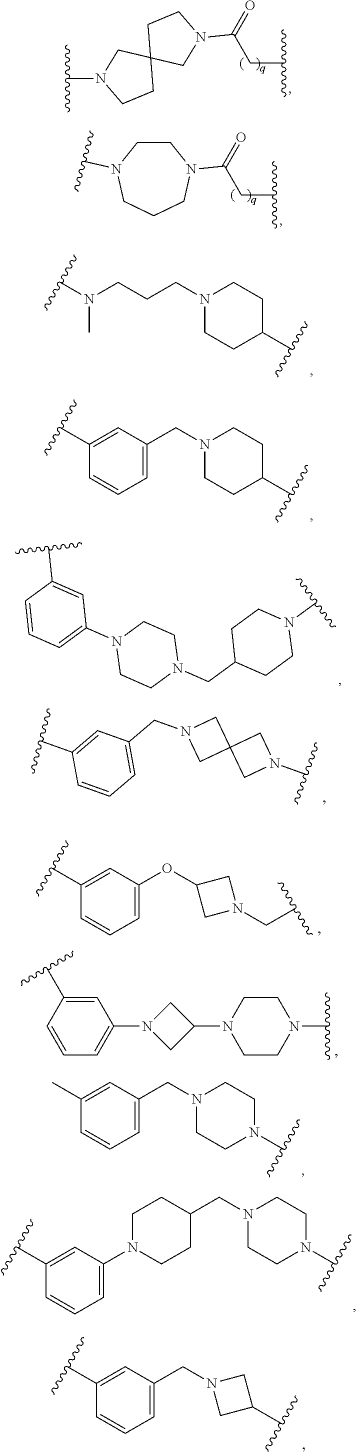

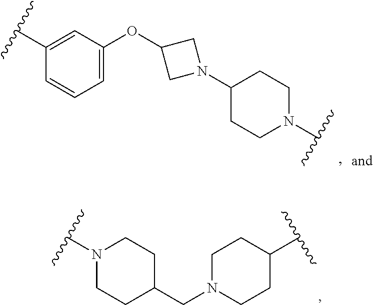

wherein q is as defined in any of the embodiments described herein and wherein the left attachment point connects to —S(O)₂— and the right attachment point connects to LBM.

In an embodiment, L is selected from

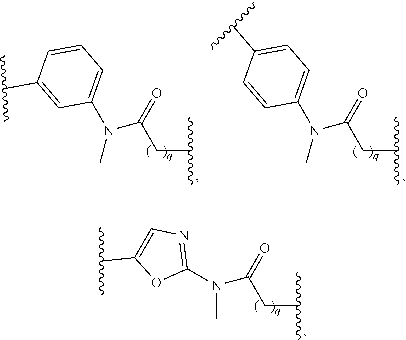

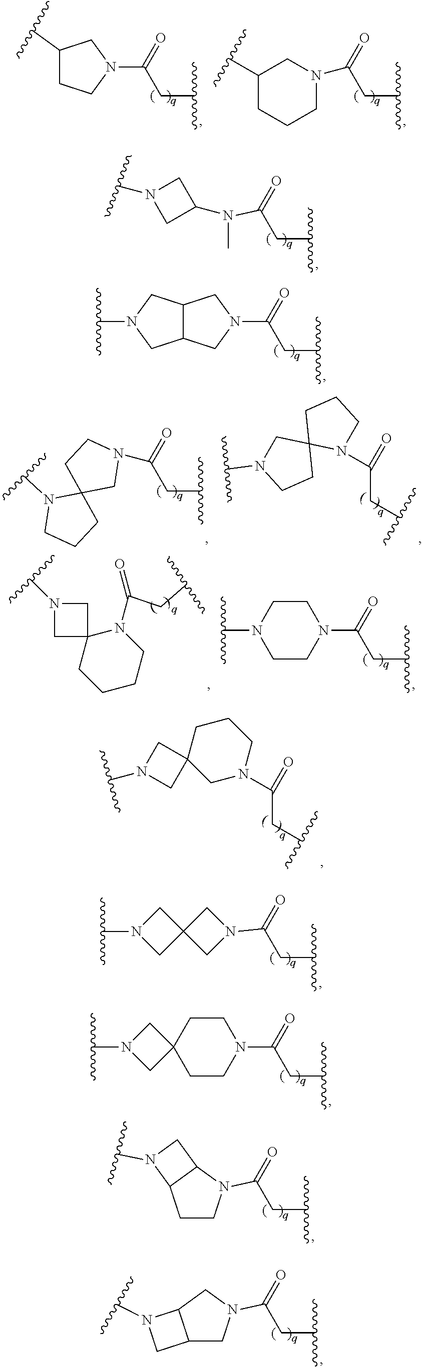

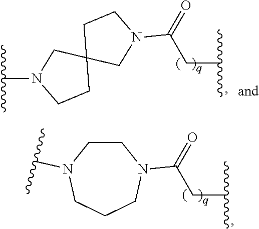

wherein q is as defined in any of the embodiments described herein and wherein the left attachment point connects to —S(O)₂— and the right attachment point connects to LBM.

In an embodiment, L is selected from

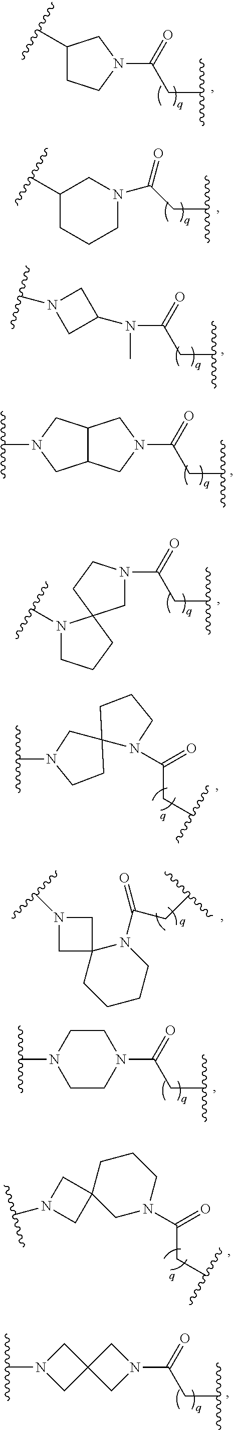

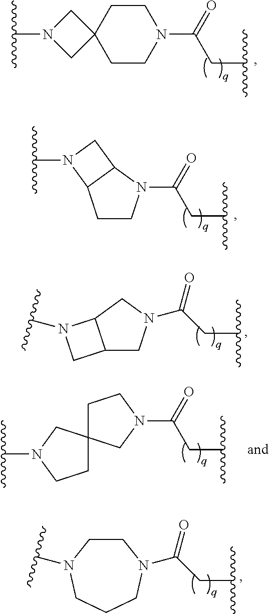

wherein q is as defined in any of the embodiments described herein and wherein the left attachment point connects to —S(O)₂— and the right attachment point connects to LBM.

In an embodiment, L is

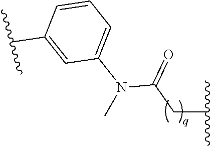

wherein q is as defined in any of the embodiments described herein and wherein the left attachment point connects to —S(O)₂— and the right attachment point connects to LBM. In an embodiment, L is

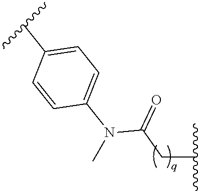

wherein q is as defined in any of the embodiments described herein and wherein the left attachment point connects to —S(O)₂— and the right attachment point connects to LBM. In an embodiment, L is

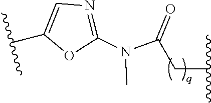

wherein q is as defined in any of the embodiments described herein and wherein the left attachment point connects to —S(O)₂— and the right attachment point connects to LBM. In an embodiment, L is

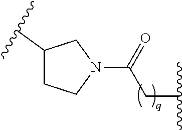

wherein q is as defined in any of the embodiments described herein and wherein the left attachment point connects to —S(O)₂— and the right attachment point connects to LBM. In an embodiment, L is

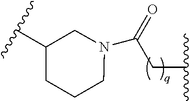

wherein q is as defined in any of the embodiments described herein and wherein the left attachment point connects to —S(O)₂— and the right attachment point connects to LBM. In an embodiment, L is

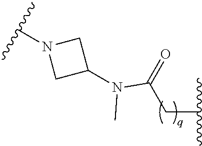

wherein q is as defined in any of the embodiments described herein and wherein the left attachment point connects to —S(O)₂— and the right attachment point connects to LBM. In an embodiment, L is

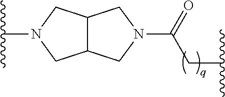

wherein q is as defined in any of the embodiments described herein and wherein the left attachment point connects to —S(O)₂— and the right attachment point connects to LBM. In an embodiment, L is

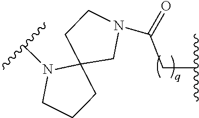

wherein q is as defined in any of the embodiments described herein and wherein the left attachment point connects to —S(O)₂— and the right attachment point connects to LBM. In an embodiment, L is

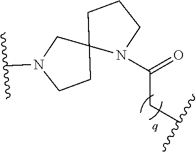

wherein q is as defined in any of the embodiments described herein and wherein the left attachment point connects to —S(O)₂— and the right attachment point connects to LBM. In an embodiment, L is

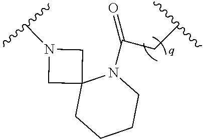

wherein q is as defined in any of the embodiments described herein and wherein the left attachment point connects to —S(O)₂— and the right attachment point connects to LBM. In an embodiment, L is

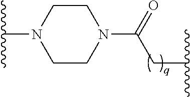

wherein q is as defined in any of the embodiments described herein and wherein the left attachment point connects to —S(O)₂— and the right attachment point connects to LBM. In an embodiment, L is

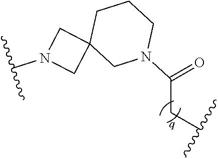

wherein q is as defined in any of the embodiments described herein and wherein the left attachment point connects to —S(O)₂— and the right attachment point connects to LBM. In an embodiment, L is

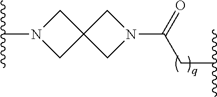

wherein q is as defined in any of the embodiments described herein and wherein the left attachment point connects to —S(O)₂— and the right attachment point connects to LBM. In an embodiment, L is

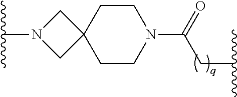

wherein q is as defined in any of the embodiments described herein. In an embodiment, L is

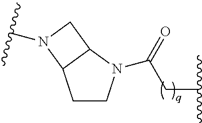

wherein q is as defined in any of the embodiments described herein and wherein the left attachment point connects to —S(O)₂— and the right attachment point connects to LBM. In an embodiment, L is

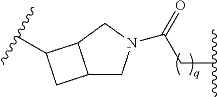

wherein q is as defined in any of the embodiments described herein and wherein the left attachment point connects to —S(O)₂— and the right attachment point connects to LBM. In an embodiment, L is

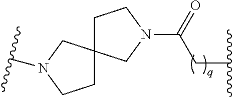

wherein q is as defined in any of the embodiments described herein and wherein the left attachment point connects to —S(O)₂— and the right attachment point connects to LBM. In an embodiment, L is

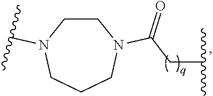

wherein q is as defined in any of the embodiments described herein and wherein the left attachment point connects to —S(O)₂— and the right attachment point connects to LBM.

As generally defined herein, each -Cy- is independently a bivalent ring selected from phenylene, an 8-10 membered bicyclic arylene, a 4-7 membered monocyclic carbocyclylene, a 5-11 membered spiro carbocyclylene, a 4-10 membered bicyclic carbocyclylene, a 5-10 membered bridged carbocyclylene, a 4-7 membered monocyclic heterocyclylene having 1-2 heteroatoms independently selected from nitrogen, oxygen, and sulfur, a 5-11 membered spiro heterocyclylene having 1-2 heteroatoms independently selected from nitrogen, oxygen, and sulfur, an 4-10 membered bicyclic heterocyclylene having 1-2 heteroatoms independently selected from nitrogen, oxygen, and sulfur, a 5-10 membered bridged bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur, wherein each phenylene, arylene, carbocyclylene, heterocyclylene and heteroarylene is substituted with 0, 1, 2, 3, or 4 instances of Re, wherein Re is as defined in any of the embodiments described herein.

In an embodiment, each -Cy- is independently a bivalent ring selected from phenylene, a 4-7 membered monocyclic heterocyclylene having 1-2 heteroatoms independently selected from nitrogen, oxygen, and sulfur, a 5-11 membered spiro heterocyclylene having 1-2 heteroatoms independently selected from nitrogen, oxygen, and sulfur, a 4-10 membered bicyclic heterocyclylene having 1-2 heteroatoms independently selected from nitrogen, oxygen, and sulfur and a 5-6 membered heteroarylene having 1-4 heteroatoms independently selected from nitrogen, oxygen, and sulfur, wherein each phenylene, heterocyclylene and heteroarylene is substituted with 0, 1, 2, 3, or 4 instances of R^{C}, wherein R^{C} is as defined in any of the embodiments described herein.

In an embodiment, each -Cy- is independently a phenylene substituted with 0, 1, 2, 3, or 4 instances of R^{C}, wherein R^{C} is as defined in any of the embodiments described herein. In an embodiment, each -Cy- is independently a 4-7 membered monocyclic heterocyclylene having 1-2 heteroatoms independently selected from nitrogen, oxygen, and sulfur, substituted with 0, 1, 2, 3, or 4 instances of R^{C}, wherein R^{C} is as defined in any of the embodiments described herein. In an embodiment, each -Cy- is a 5-11 membered spiro heterocyclylene having 1-2 heteroatoms independently selected from nitrogen, oxygen, and sulfur, substituted with 0, 1, 2, 3, or 4 instances of R^{C}, wherein R^{C} is as defined in any of the embodiments described herein. In an embodiment, each -Cy- is a 4-10 membered bicyclic heterocyclylene having 1-2 heteroatoms independently selected from nitrogen, oxygen, and sulfur, substituted with 0, 1, 2, 3, or 4 instances of R^{C}, wherein R^{C} is as defined in any of the embodiments described herein. In an embodiment, each -Cy- is a 5-6 membered heteroarylene having 1-4 heteroatoms independently selected from nitrogen, oxygen, and sulfur, substituted with 0, 1, 2, 3, or 4 instances of R^{C}, wherein R^{C} is as defined in any of the embodiments described herein.

In an embodiment, Cy is selected from:

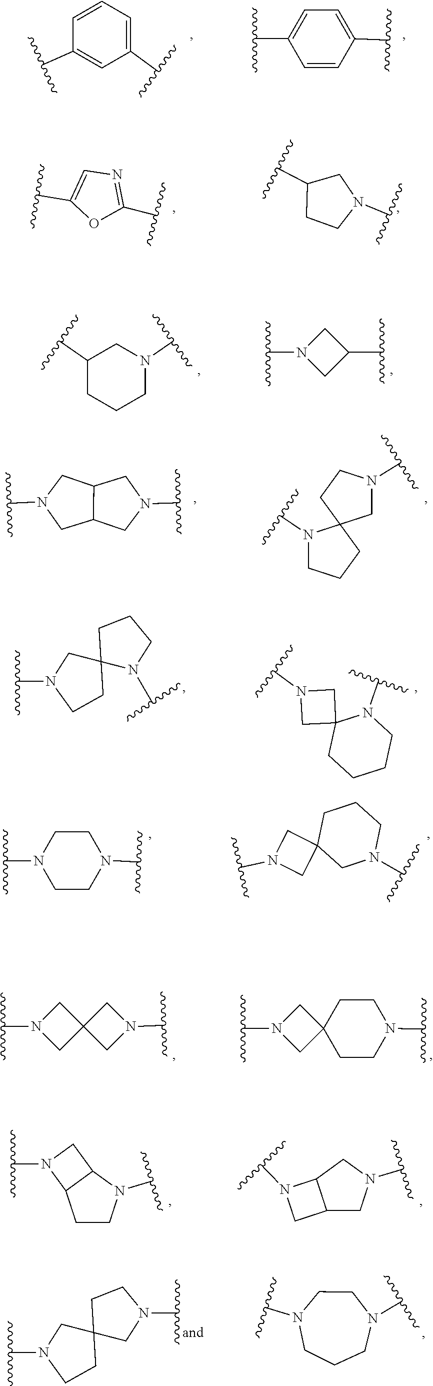

and each substituted with 0, 1, 2, 3, or 4 instances of R^{C}, wherein R^{C} is as defined in any of the embodiments described herein and wherein the left side attachment point connects to L¹ group and the right side attachment point connects to L².

In an embodiment, Cy is selected from:

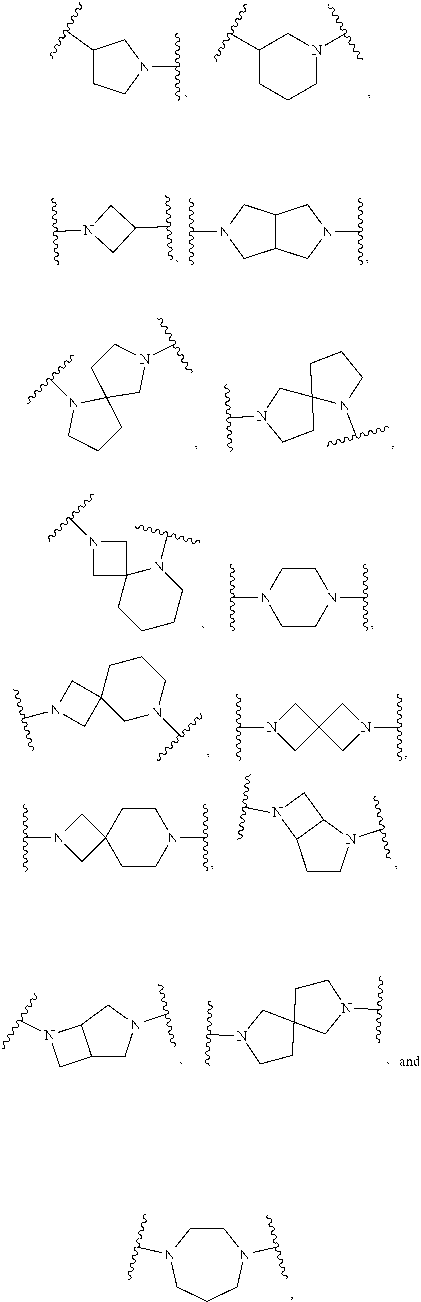

each substituted with 0, 1, 2, 3, or 4 instances of R^{C}, wherein R^{C} is as defined in any of the embodiments described herein and wherein the left side attachment point connects to L¹ group and the right side attachment point connects to L².

In an embodiment, -Cy- is unsubstituted. In an embodiment, -Cy- is substituted with 0 or 1 instances of R^{C}. In an embodiment, -Cy- is substituted with 0, 1 or 2 instances of R^{C}. In an embodiment, -Cy- is substituted with 0, 1, 2 or 3 instances of R^{C}. In an embodiment, -Cy- is substituted with 1 instance of R^{C}. In an embodiment, -Cy- is substituted with 2 instances of R^{C}. In an embodiment, -Cy- is substituted with 3 instances of R^{C}. In an embodiment, -Cy- is substituted with 4 instances of R^{C}.

In an embodiment, Cy is selected from:

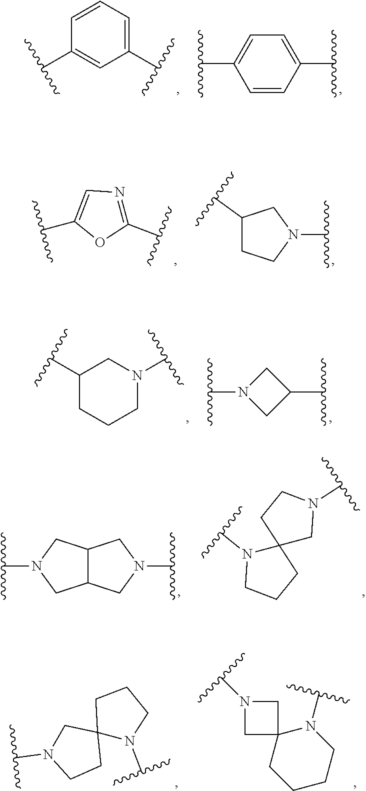

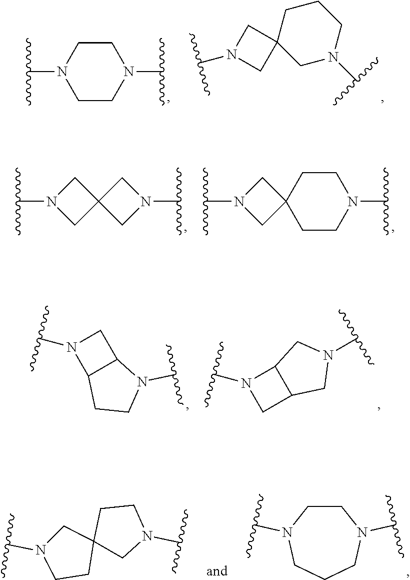

each not further substituted wherein the left side attachment point connects to L¹ group and the right side attachment point connects to L².

In an embodiment, Cy is selected from:

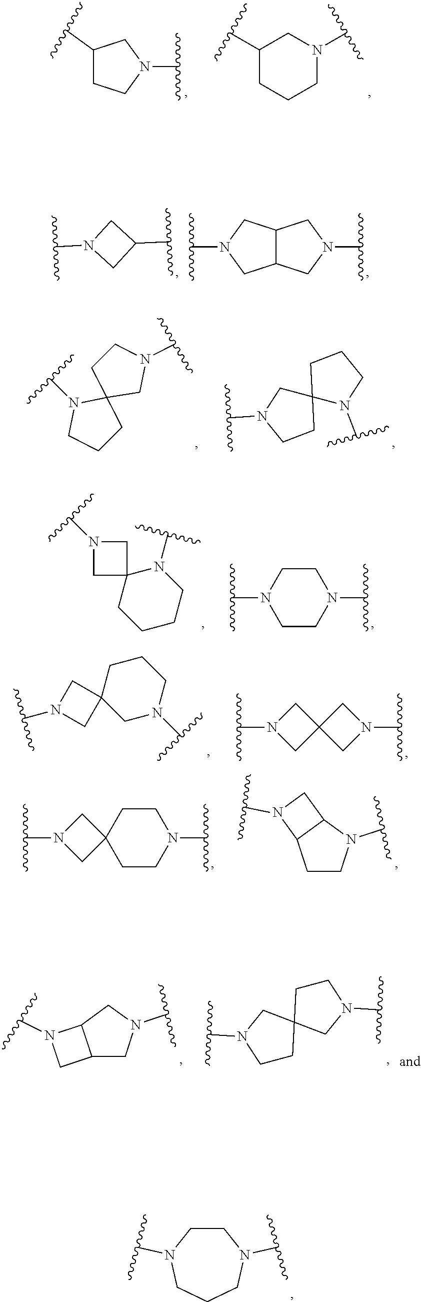

each not further substituted wherein the left side attachment point connects to L¹ group and the right side attachment point connects to L².

In an embodiment, Cy is

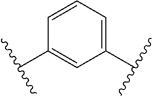 [SMILES: *c1cccc(*)c1]

wherein the left side attachment point connects to L¹ group and the right side attachment point connects to L². In an embodiment, Cy is

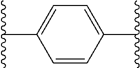 [SMILES: *c1ccc(*)cc1]

wherein the left side attachment point connects to L¹ group and the right side attachment point connects to L². In an embodiment, Cy is

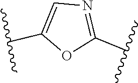 [SMILES: *c1cnc(*)o1]

wherein the left side attachment point connects to L¹ group and the right side attachment point connects to L². In an embodiment, Cy is

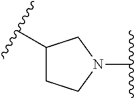 [SMILES: *C1CCN(*)C1]

wherein the left side attachment point connects to L¹ group and the right side attachment point connects to L². In an embodiment, Cy is

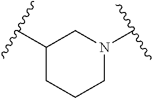 [SMILES: *C1CCCN(*)C1]

wherein the left side attachment point connects to L¹ group and the right side attachment point connects to L². In an embodiment, Cy is

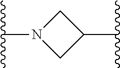 [SMILES: *C1CN(*)C1]

wherein the left side attachment point connects to L¹ group and the right side attachment point connects to L². In an embodiment, Cy is

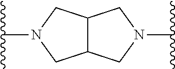 [SMILES: *N1CC2CN(*)CC2C1]

wherein the left side attachment point connects to L¹ group and the right side attachment point connects to L². In an embodiment, Cy is

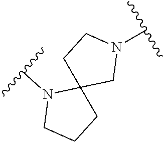 [SMILES: *N1CCC2(CCCN2*)C1]

wherein the left side attachment point connects to L¹ group and the right side attachment point connects to L². In an embodiment, Cy is

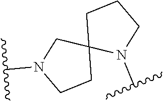 [SMILES: *N1CCC2(CCCN2*)C1]

wherein the left side attachment point connects to L¹ group and the right side attachment point connects to L². In an embodiment, Cy is

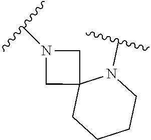 [SMILES: *N1CC2(CCCCN2*)C1]

wherein the left side attachment point connects to L¹ group and the right side attachment point connects to L². In an embodiment, Cy is

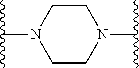 [SMILES: *N1CCN(*)CC1]

wherein the left side attachment point connects to L¹ group and the right side attachment point connects to L². In an embodiment, Cy is

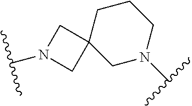 [SMILES: *N1CCCC2(C1)CN(*)C2]

wherein the left side attachment point connects to L¹ group and the right side attachment point connects to L². In an embodiment, Cy is

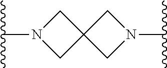 [SMILES: *N1CC2(C1)CN(*)C2]

wherein the left side attachment point connects to L¹ group and the right side attachment point connects to L². In an embodiment, Cy is

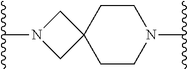 [SMILES: *N1CCC2(CC1)CN(*)C2]

wherein the left side attachment point connects to L¹ group and the right side attachment point connects to L². In an embodiment, Cy is

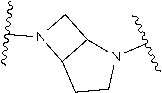 [SMILES: *N1CCC2C1CN2*]

wherein the left side attachment point connects to L¹ group and the right side attachment point connects to L². In an embodiment, Cy is

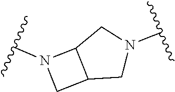 [SMILES: *N1CC2CN(*)C2C1]

wherein the left side attachment point connects to L¹ group and the right side attachment point connects to L². In an embodiment, Cy is

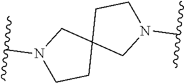 [SMILES: *N1CCC2(CCN(*)C2)C1]

wherein the left side attachment point connects to L¹ group and the right side attachment point connects to L². In an embodiment, Cy is

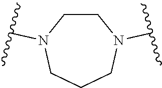 [SMILES: *N1CCCN(*)CC1]

wherein the left side attachment point connects to L¹ group and the right side attachment point connects to L².

As generally defined herein, LBM is selected from

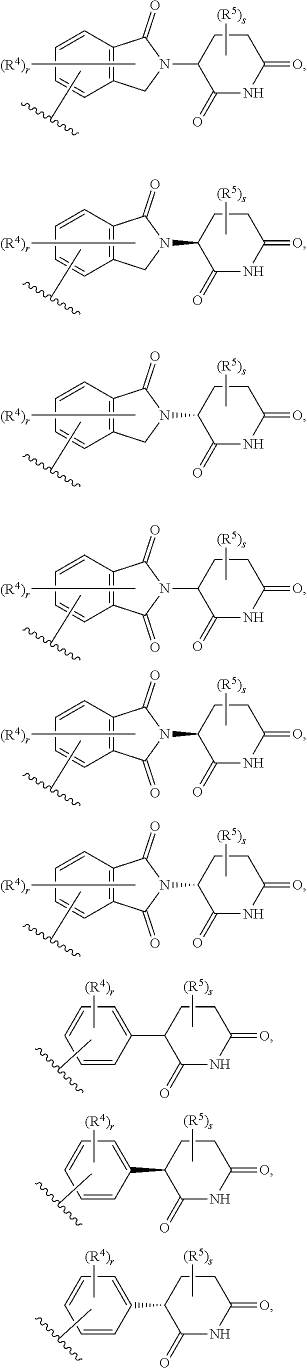

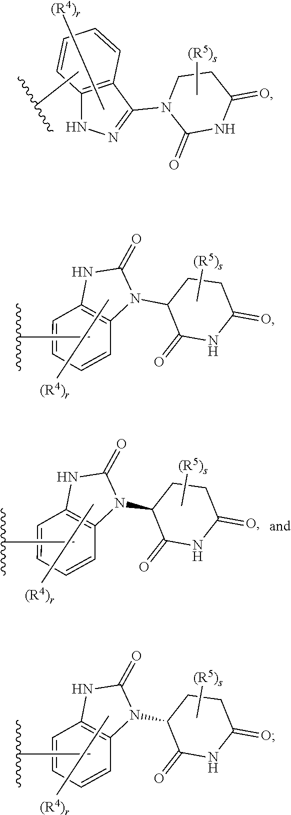

wherein each R⁴, R⁵, r and s is as defined in any of the embodiments described herein.

In an embodiment, LBM is selected from

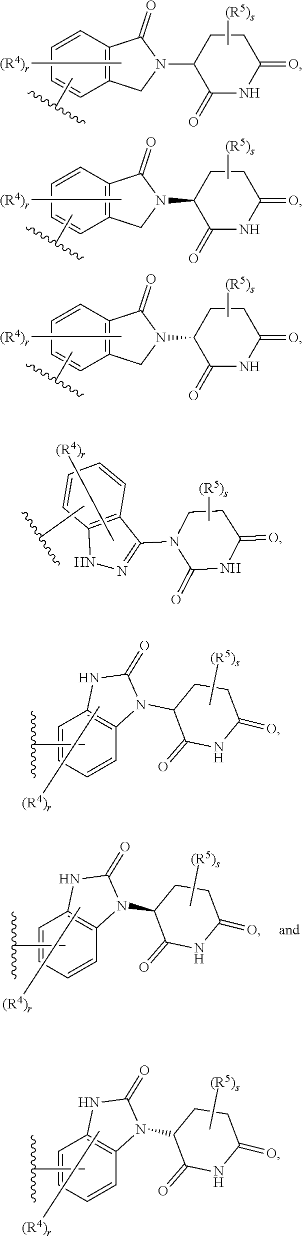

wherein each R⁴, R⁵, r and s is as defined in any of the embodiments described herein.

In an embodiment, LBM is

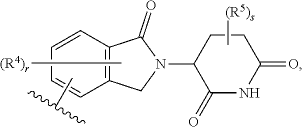

wherein each R⁴, R⁵, r and s is as defined in any of the embodiments described herein. In an embodiment, LBM is

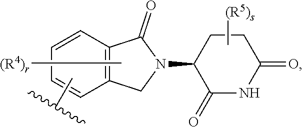

wherein each R⁴, R, r and s is as defined in any of the embodiments described herein. In an embodiment, LBM is

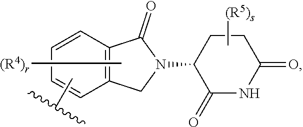

wherein each R⁴, R⁵, r and s is as defined in any of the embodiments described herein. In an embodiment, LBM is

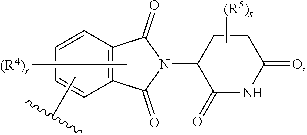

wherein each R⁴, R, r and s is as defined in any of the embodiments described herein. In an embodiment, LBM is

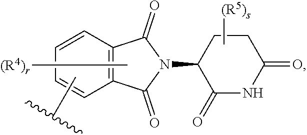

wherein each R⁴, R⁵, r and s is as defined in any of the embodiments described herein. In an embodiment, LBM is

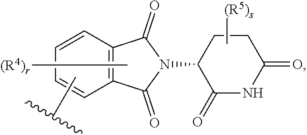

wherein each R⁴, R⁵, r and s is as defined in any of the embodiments described herein. In an embodiment, LBM is

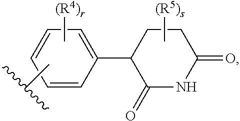

wherein each R⁴, R⁵, r and s is as defined in any of the embodiments described herein. In an embodiment, LBM is

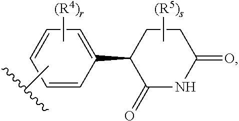

wherein each R⁴, R⁵, r and s is as defined in any of the embodiments described herein. In an embodiment, LBM is

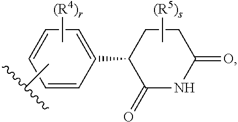

wherein each R⁴, R⁵, r and s is as defined in any of the embodiments described herein. In an embodiment, LBM is

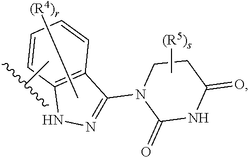

wherein each R⁴, R⁵, r and s is as defined in any of the embodiments described herein. In an embodiment, LBM is

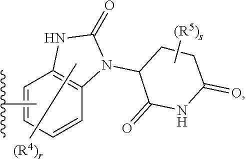

wherein each R⁴, R⁵, r and s is as defined in any of the embodiments described herein. In an embodiment, LBM is

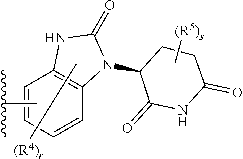

wherein each R⁴, R, r and s is as defined in any of the embodiments described herein. In an embodiment, LBM is

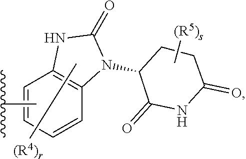

wherein each R⁴, R⁵, r and s is as defined in any of the embodiments described herein.

In an embodiment, LBM is selected from

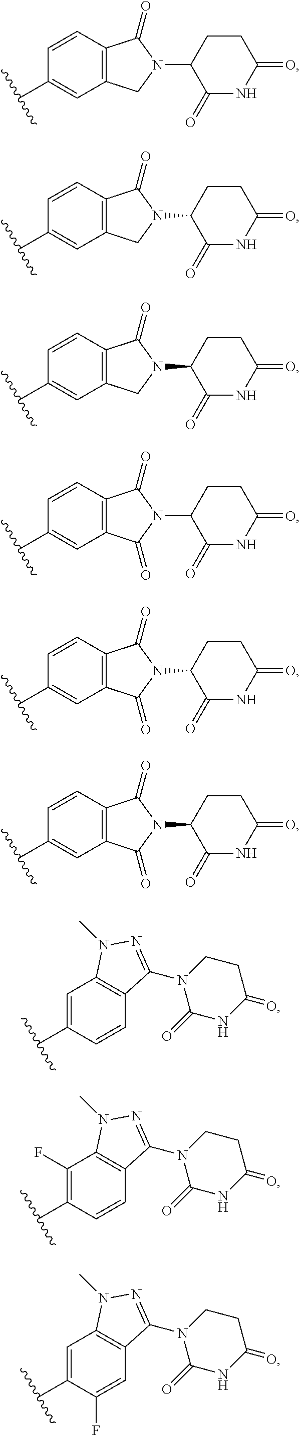

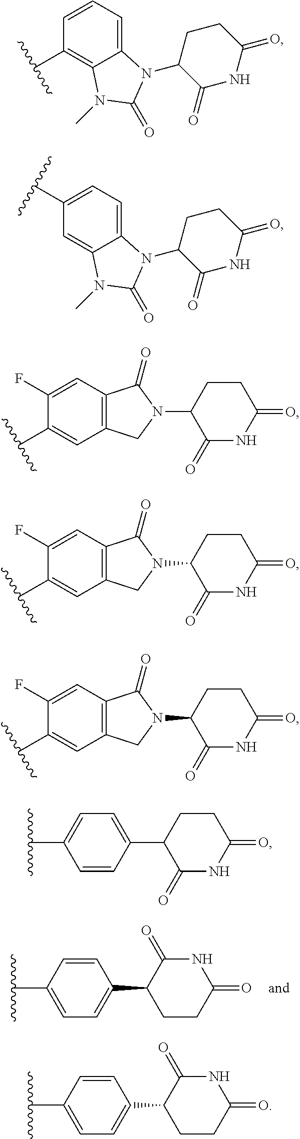

In an embodiment, LBM is selected from

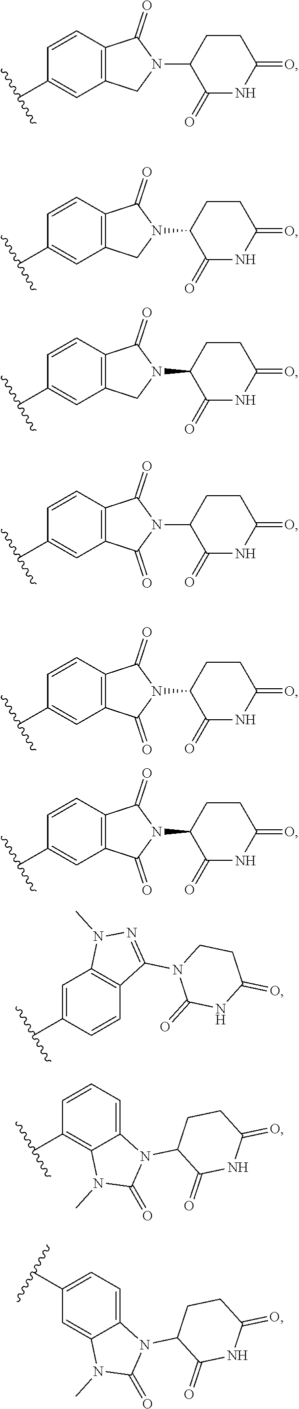

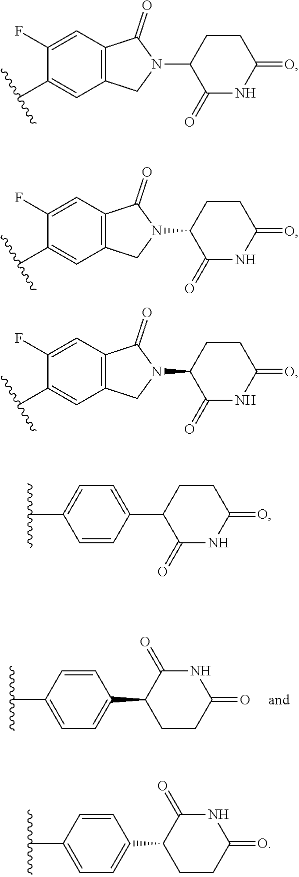

In an embodiment, LBM is selected from

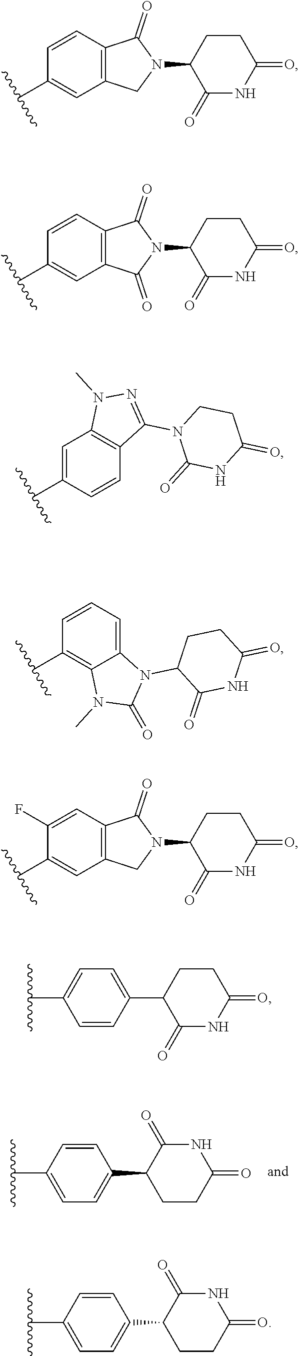

In an embodiment, LBM is selected from

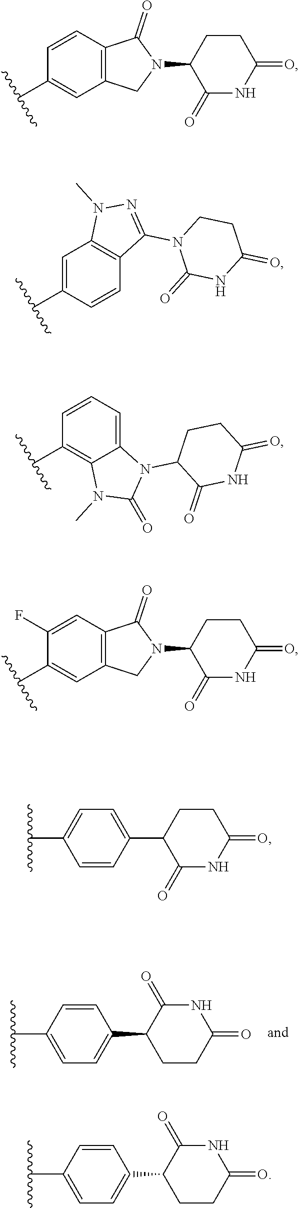

In an embodiment, LBM is selected from

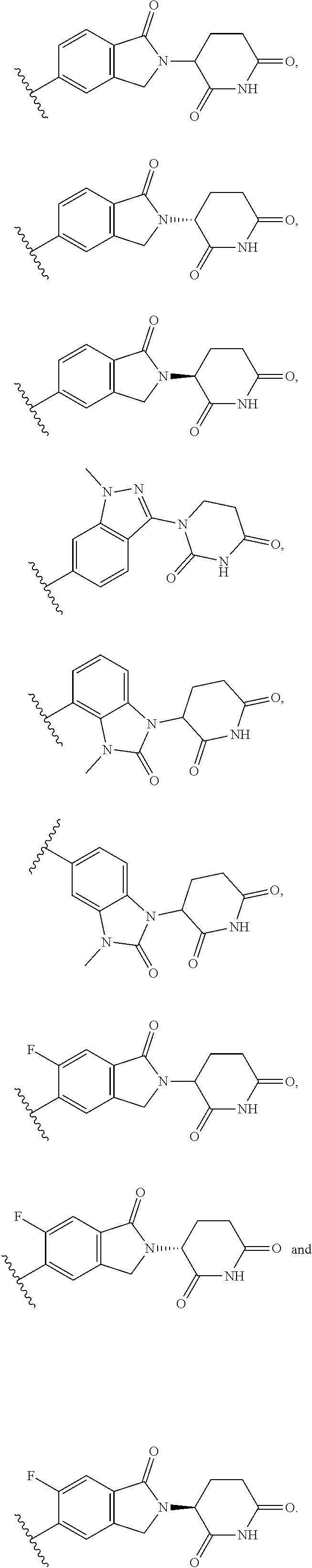

In an embodiment, LBM is selected from

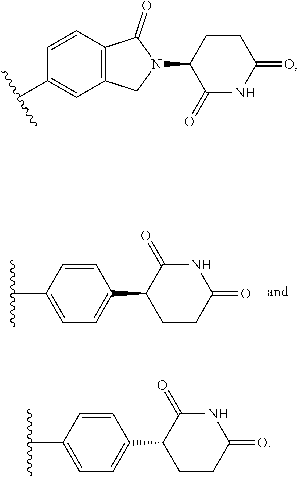 [SMILES: *c1ccc2c(c1)CN([C@H]1CCC(=O)NC1=O)C2=O ; *c1ccc([C@@H]2CCC(=O)NC2=O)cc1 ; *c1ccc([C@H]2CCC(=O)NC2=O)cc1]

In an embodiment, LBM is selected from

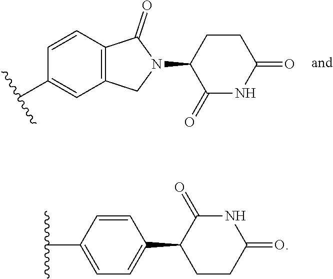 [SMILES: *c1ccc2c(c1)CN([C@H]1CCC(=O)NC1=O)C2=O ; *c1ccc([C@@H]2CCC(=O)NC2=O)cc1]

In an embodiment, LBM is selected from

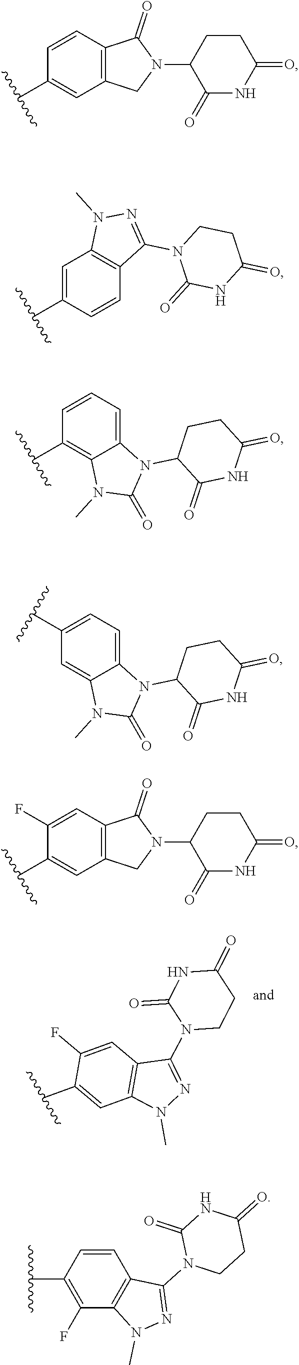

In an embodiment, LBM is

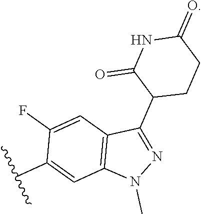 [SMILES: *c1cc2c(cc1F)c(C1CCC(=O)NC1=O)nn2C]

In an embodiment, LBM is

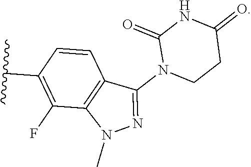 [SMILES: *c1ccc2c(N3CCC(=O)NC3=O)nn(C)c2c1F]

In an embodiment, LBM is

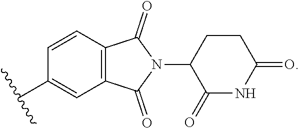 [SMILES: *c1ccc2c(c1)C(=O)N(C1CCC(=O)NC1=O)C2=O]

In an embodiment, LBM is

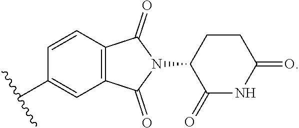 [SMILES: *c1ccc2c(c1)C(=O)N([C@@H]1CCC(=O)NC1=O)C2=O]

In an embodiment, LBM is

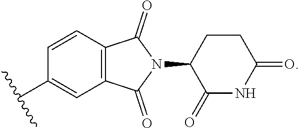 [SMILES: *c1ccc2c(c1)C(=O)N([C@H]1CCC(=O)NC1=O)C2=O]

In an embodiment, LBM is

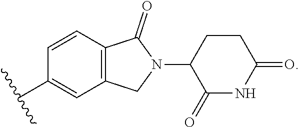 [SMILES: *c1ccc2c(c1)CN(C1CCC(=O)NC1=O)C2=O]

In an embodiment, LBM is

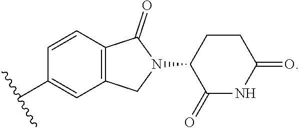 [SMILES: *c1ccc2c(c1)CN([C@@H]1CCC(=O)NC1=O)C2=O]

In an embodiment, LBM is

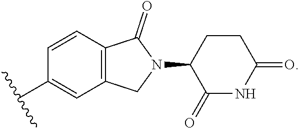 [SMILES: *c1ccc2c(c1)CN([C@H]1CCC(=O)NC1=O)C2=O]

In an embodiment, LBM is

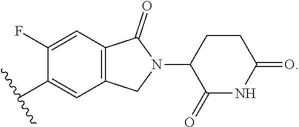 [SMILES: *c1cc2c(cc1F)C(=O)N(C1CCC(=O)NC1=O)C2]

In an embodiment, LBM is

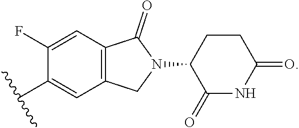 [SMILES: *c1cc2c(cc1F)C(=O)N([C@@H]1CCC(=O)NC1=O)C2]

In an embodiment, LBM is

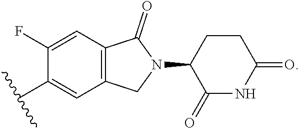 [SMILES: *c1cc2c(cc1F)C(=O)N([C@H]1CCC(=O)NC1=O)C2]

In an embodiment, LBM is

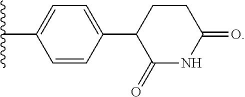 [SMILES: *c1ccc(C2CCC(=O)NC2=O)cc1]

In an embodiment, LBM is

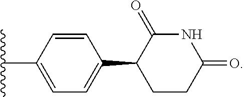 [SMILES: *c1ccc([C@@H]2CCC(=O)NC2=O)cc1]

In an embodiment, LBM is

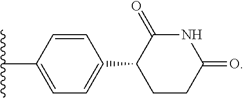 [SMILES: *c1ccc([C@H]2CCC(=O)NC2=O)cc1]

In an embodiment, LBM is

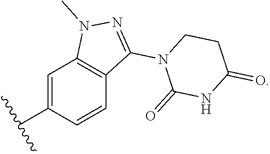 [SMILES: *c1ccc2c(N3CCC(=O)NC3=O)nn(C)c2c1]

In an embodiment, LBM is

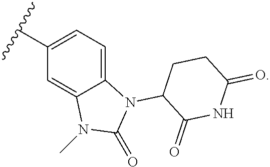 [SMILES: *c1ccc2c(c1)n(C)c(=O)n2C1CCC(=O)NC1=O]

In an embodiment, LBM is

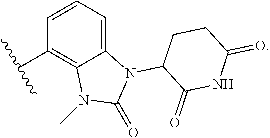 [SMILES: *c1cccc2c1n(C)c(=O)n2C1CCC(=O)NC1=O]

As generally defined herein, each instance of R^{A} is independently selected from oxo, deuterium, halogen, —C₁₋₆ alkyl, —C₁₋₆ heteroalkyl, —C₁₋₆ haloalkyl, —C₂₋₆ alkenyl, —C₂₋₆ alkynyl, —CN, —NO₂, —OR, —SR, —NR₂, —S(O)₂R, —S(O)₂NR₂, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, wherein each R is as defined in any of the embodiments described herein.

In an embodiment, each instance of R^{A} is independently selected from oxo, deuterium, halogen, —C₁₋₆ alkyl, —C₁₋₆ heteroalkyl, —C₁₋₆ haloalkyl, —C₂₋₆ alkenyl, —C₂₋₆ alkynyl, —CN, —OR, —SR, —NR₂, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, wherein each R is as defined in any of the embodiments described herein.

In an embodiment, each instance of R^{A} is independently selected from oxo, deuterium, halogen, —C₁₋₆ alkyl, —C₁₋₆ heteroalkyl, —C₁₋₆ haloalkyl, —C₂₋₆ alkynyl, —CN, —OR, —NR₂, —C₃₋₇ cycloalkyl and 4-7 membered heterocyclyl, wherein said each heterocyclyl and heteroaryl contains 1 or 2 heteroatoms independently selected from N and O, wherein each R is as defined in any of the embodiments described herein.

In an embodiment, each instance of R^{A} is independently selected from oxo, deuterium, halogen, —C₁₋₆ alkyl, —C₁₋₆ haloalkyl, —OR, —C₃₋₇ cycloalkyl and 4-7 membered heterocyclyl, wherein said each heterocyclyl and heteroaryl contains 1 or 2 heteroatoms independently selected from N and O, wherein each R is as defined in any of the embodiments described herein.

In an embodiment, each instance of R^{A} is independently selected from oxo, deuterium, —F, —Cl, -Me, -Et, —Pr, —ⁱPr, -^{t}Bu, —CF₃, —OH, —OMe, cyclopropyl, cyclobutyl, azetidinyl and oxetanyl.

In an embodiment, each instance of R^{A} is independently selected from oxo, deuterium, —F, and -Me.

In an embodiment, each instance of R^{A} is independently selected from halogen and —C₁₋₆ alkyl.

In an embodiment, each instance of R^{A} is independently selected from —F and -Me.

In an embodiment, R^{A} is —F.

In an embodiment, the moiety represented by

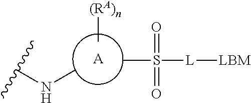

or by

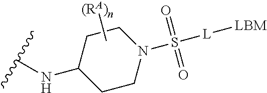

is selected from

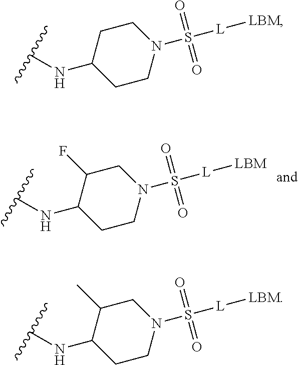

In an embodiment, the moiety represented by

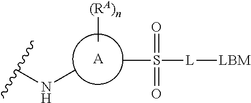

or by

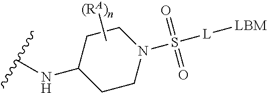

is

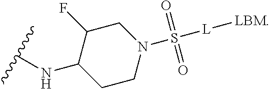

In an embodiment, the moiety represented by

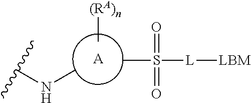

or by

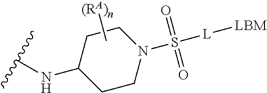

is

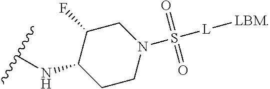

In an embodiment, the —NH— and F— are in a cis configuration.

In an embodiment, the —NH— and F— are in a trans configuration.

In an embodiment, the moiety represented by

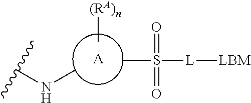

or by

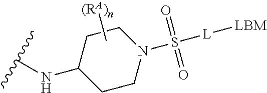

is

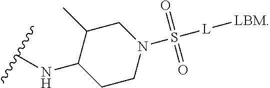

In an embodiment, the —NH— and Me- are in a cis configuration.

In an embodiment, the —NH— and Me- are in a trans configuration.

In an embodiment, the moiety represented by

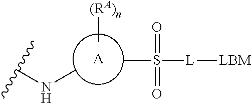

or by

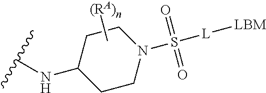

is selected from

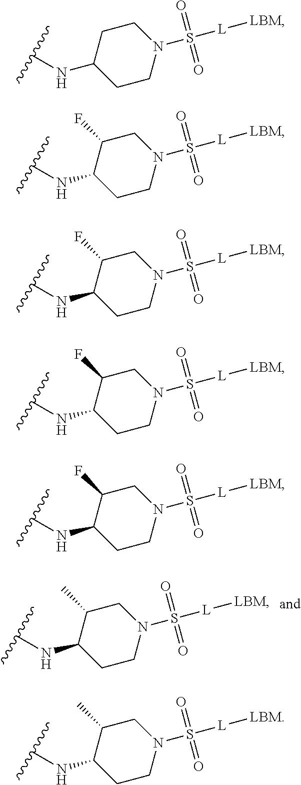

In an embodiment, the moiety represented by

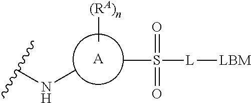

or by

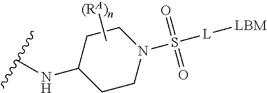

is selected from

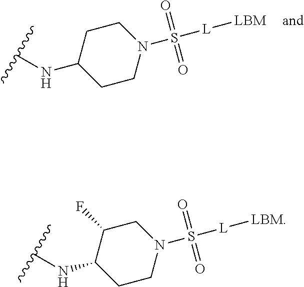

In an embodiment, the moiety represented by

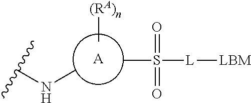

or by

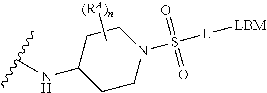

is

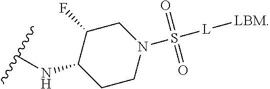

In an embodiment, the moiety represented by

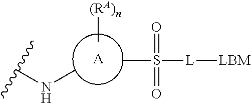

or by

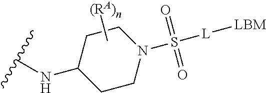

is

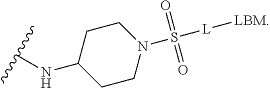

As generally defined herein, each instance of R^{B} Is independently selected from oxo, deuterium, halogen, —C₁₋₆ alkyl, —C₁₋₆ heteroalkyl, —C₁₋₆ haloalkyl, —C₂₋₆ alkenyl, —C₂₋₆ alkynyl, —CN, —NO₂, —OR, —SR, —NR₂, —S(O)₂R, —S(O)₂NR₂, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, wherein each R is as defined in any of the embodiments described herein.

In an embodiment, each instance of R^{B} is independently selected from oxo, deuterium, halogen, —C₁₋₆ alkyl, —C₁₋₆ heteroalkyl, —C₁₋₆ haloalkyl, —C₂₋₆ alkenyl, —C₂₋₆ alkynyl, —CN, —OR, —SR, —NR₂, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, wherein each R is as defined in any of the embodiments described herein.

In an embodiment, each instance of R^{B} is independently selected from oxo, deuterium, halogen, —C₁₋₆ alkyl, —C₁₋₆ heteroalkyl, —C₁₋₆ haloalkyl, —C₂₋₆ alkynyl, —CN, —OR, —NR₂, —C₃₋₇ cycloalkyl and 4-7 membered heterocyclyl, wherein said each heterocyclyl and heteroaryl contains 1 or 2 heteroatoms independently selected from N and O, wherein each R is as defined in any of the embodiments described herein.

In an embodiment, each instance of R^{B} is independently selected from oxo, deuterium, halogen, —C₁₋₆ alkyl, —C₁₋₆ haloalkyl, —OR, —C₃₋₇ cycloalkyl and 4-7 membered heterocyclyl, wherein said each heterocyclyl and heteroaryl contains 1 or 2 heteroatoms independently selected from N and O, wherein each R is as defined in any of the embodiments described herein.

In an embodiment, each instance of R^{B} is independently selected from deuterium, —F, —Cl, -Me, -Et, —Pr, —ⁱPr, -^{t}Bu, —CF₃, —OH, —OMe, cyclopropyl, cyclobutyl, azetidinyl and oxetanyl.

In an embodiment, each instance of R^{B} is independently selected from deuterium, —F, and -Me.

As generally defined herein, each instance of R^{C} is independently selected from oxo, deuterium, halogen, —C₁₋₆ alkyl, —C₁₋₆ heteroalkyl, —C₁₋₆ haloalkyl, —C₂₋₆ alkenyl, —C₂₋₆ alkynyl, —CN, —NO₂, —OR, —SR, —NR₂, —S(O)₂R, —S(O)₂NR₂, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, wherein each R is as defined in any of the embodiments described herein.

In an embodiment, each instance of R^{C} is independently selected from oxo, deuterium, halogen, —C₁₋₆ alkyl, —C₁₋₆ heteroalkyl, —C₁₋₆ haloalkyl, —C₂₋₆ alkenyl, —C₂₋₆ alkynyl, —CN, —OR, —SR, —NR₂, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, wherein each R is as defined in any of the embodiments described herein.

In an embodiment, each instance of R^{C} is independently selected from oxo, deuterium, halogen, —C₁₋₆ alkyl, —C₁₋₆ heteroalkyl, —C₁₋₆ haloalkyl, —C₂₋₆ alkynyl, —CN, —OR, —NR₂, —C₃₋₇ cycloalkyl and 4-7 membered heterocyclyl, wherein said each heterocyclyl and heteroaryl contains 1 or 2 heteroatoms independently selected from N and O, wherein each R is as defined in any of the embodiments described herein.

In an embodiment, each instance of R^{C} is independently selected from oxo, deuterium, halogen, —C₁₋₆ alkyl, —C₁₋₆ haloalkyl, —OR, —C₃₋₇ cycloalkyl and 4-7 membered heterocyclyl, wherein said each heterocyclyl and heteroaryl contains 1 or 2 heteroatoms independently selected from N and O, wherein each R is as defined in any of the embodiments described herein.

In an embodiment, each instance of R^{C} is independently selected from oxo, deuterium, —F, —Cl, -Me, -Et, —Pr, —ⁱPr, -Bu, —CF₃, —OH, —OMe, cyclopropyl, cyclobutyl, azetidinyl and oxetanyl.

In an embodiment, each instance of R^{C} is independently selected from oxo, deuterium, —F, and -Me.

As generally defined herein, each instance of R⁴ is independently selected from oxo, deuterium, halogen, —C₁₋₆ alkyl, —C₁₋₆ heteroalkyl, —C₁₋₆ haloalkyl, —C₂₋₆ alkenyl, —C₂₋₆ alkynyl, —CN, —NO₂, —OR, —SR, —NR₂, —S(O)₂R, —S(O)₂NR₂, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, wherein each R is as defined in any of the embodiments described herein.

In an embodiment, each instance of R⁴ is independently selected from oxo, deuterium, halogen, —C₁₋₆ alkyl, —C₁₋₆ heteroalkyl, —C₁₋₆ haloalkyl, —C₂₋₆ alkenyl, —C₂₋₆ alkynyl, —CN, —OR, —SR, —NR₂, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, wherein each R is as defined in any of the embodiments described herein.

In an embodiment, each R⁴ is independently selected from deuterium, halogen, —C₁₋₆ alkyl, —C₁₋₆ haloalkyl, —CN, —OR, —NR₂, —S(O)₂R, —S(O)₂NR₂, —S(O)R, —S(O)NR₂, —S(O)(NR)R, —C(O)NR₂, —C(O)N(R)OR, —OC(O)NR₂, —N(R)C(O)OR, —N(R)C(O)R, —N(R)C(O)NR₂, —N(R)S(O)₂NR₂ and —N(R)S(O)₂R, wherein R is H or C₁₋₆ alkyl.

In an embodiment, each instance of R⁴ is independently selected from oxo, deuterium, halogen, —C₁₋₆ alkyl, —C₁₋₆ heteroalkyl, —C₁₋₆ haloalkyl, —C₂₋₆ alkynyl, —CN, —OR, —NR₂, —C₃₋₇ cycloalkyl and 4-7 membered heterocyclyl, wherein said each heterocyclyl and heteroaryl contains 1 or 2 heteroatoms independently selected from N and O, wherein each R is as defined in any of the embodiments described herein.

In an embodiment, each instance of R⁴ is independently selected from oxo, deuterium, halogen, —C₁₋₆ alkyl, —C₁₋₆ haloalkyl, —OR, —C₃₋₇ cycloalkyl and 4-7 membered heterocyclyl, wherein said each heterocyclyl and heteroaryl contains 1 or 2 heteroatoms independently selected from N and O, wherein each R is as defined in any of the embodiments described herein.

In an embodiment, each R⁴ is independently selected from -Me, -Et, —F, —Cl, —CF₃, —CN, —OH, —OMe, —NH₂, —NHMe and —NMe₂.

In an embodiment, each instance of R⁴ is independently selected from deuterium, —F, —Cl, -Me, -Et, —Pr, —ⁱPr, -^{t}Bu, —CF₃, —OH, —OMe, cyclopropyl, cyclobutyl, azetidinyl and oxetanyl.

In an embodiment, each instance of R⁴ is independently selected from deuterium, —F, and -Me. In an embodiment, each R⁴ is independently selected from -Me and —F.

As generally defined herein, each instance of R⁵ is independently selected from oxo, deuterium, halogen, —C₁₋₆ alkyl, —C₁₋₆ heteroalkyl, —C₁₋₆ haloalkyl, —C₂₋₆ alkenyl, —C₂₋₆ alkynyl, —CN, —NO₂, —OR, —SR, —NR₂, —S(O)₂R, —S(O)₂NR₂, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, wherein each R is as defined in any of the embodiments described herein.

In an embodiment, each instance of R⁵ is independently selected from oxo, deuterium, halogen, —C₁₋₆ alkyl, —C₁₋₆ heteroalkyl, —C₁₋₆ haloalkyl, —C₂₋₆ alkynyl, —CN, —OR, —NR₂, —C₃₋₇ cycloalkyl and 4-7 membered heterocyclyl, wherein said each heterocyclyl and heteroaryl contains 1 or 2 heteroatoms independently selected from N and O, wherein each R is as defined in any of the embodiments described herein.

In an embodiment, each instance of R⁵ is independently selected from oxo, deuterium, halogen, —C₁₋₆ alkyl, —C₁₋₆ haloalkyl, —OR, —C₃₋₇ cycloalkyl and 4-7 membered heterocyclyl, wherein said each heterocyclyl and heteroaryl contains 1 or 2 heteroatoms independently selected from N and O, wherein each R is as defined in any of the embodiments described herein.

In an embodiment, each R⁵ is independently selected from -Me, -Et, —F, —Cl, —CF₃, —CN, —OH, —OMe, —NH₂, —NHMe and —NMe₂.

In an embodiment, each instance of R⁵ is independently selected from deuterium, —F, —Cl, -Me, -Et, —Pr, —ⁱPr, -^{t}Bu, —CF₃, —OH, —OMe, cyclopropyl, cyclobutyl, azetidinyl and oxetanyl.

In an embodiment, each instance of R⁵ is independently selected from deuterium, —F, and -Me.

In an embodiment, each instance of R⁵ is independently selected from —F, and -Me.

As generally defined herein, each instance of R⁶ is independently selected from oxo, deuterium, halogen, —C₁₋₆ alkyl, —C₁₋₆ heteroalkyl, —C₁₋₆ haloalkyl, —C₂₋₆ alkenyl, —C₂₋₆ alkynyl, —CN, —NO₂, —OR, —SR, —NR₂, —S(O)₂R, —S(O)₂NR₂, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, wherein each R is as defined in any of the embodiments described herein.

In an embodiment, each instance of R⁶ is independently selected from oxo, deuterium, halogen, —C₁₋₆ alkyl, —C₁₋₆ heteroalkyl, —C₁₋₆ haloalkyl, —C₂₋₆ alkenyl, —C₂₋₆ alkynyl, —CN, —OR, —SR, —NR₂, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, wherein each R is as defined in any of the embodiments described herein.

In an embodiment, each instance of R⁶ is independently selected from oxo, deuterium, halogen, —C₁₋₆ alkyl, —C₁₋₆ heteroalkyl, —C₁₋₆ haloalkyl, —C₂₋₆ alkynyl, —CN, —OR, —NR₂, —C₃₋₇ cycloalkyl and 4-7 membered heterocyclyl, wherein said each heterocyclyl and heteroaryl contains 1 or 2 heteroatoms independently selected from N and O, wherein each R is as defined in any of the embodiments described herein.

In an embodiment, each instance of R⁶ is independently selected from oxo, deuterium, halogen, —C₁₋₆ alkyl, —C₁₋₆ haloalkyl, —OR, —C₃₋₇ cycloalkyl and 4-7 membered heterocyclyl, wherein said each heterocyclyl and heteroaryl contains 1 or 2 heteroatoms independently selected from N and O, wherein each R is as defined in any of the embodiments described herein.

In an embodiment, R⁶ is selected from halo, —OH and —C₁₋₆ alkyl.

In an embodiment, each instance of R⁶ is independently selected from oxo, deuterium, —F, —Cl, -Me, -Et, —Pr, —ⁱPr, -^{t}Bu, —CF₃, —OH, —OMe, cyclopropyl, cyclobutyl, azetidinyl and oxetanyl.

In an embodiment, R⁶ is selected from —F, —Cl, —OH, -Me, -Et-, —ⁱPr.

In an embodiment, each instance of R⁶ is independently selected from oxo, deuterium, —F, and -Me. In an embodiment, each instance of R⁶ is independently selected from —OH, —F and -Me

In an embodiment, R⁶ is selected from —F and -Me. In an embodiment, each instance of R⁶ is independently selected from —OH and -Me

In an embodiment, R⁶ is -Me.

In an embodiment, R⁶ is —OH.

As generally defined herein, each R⁷ is independently selected from —C₁₄ alkyl and halo. In an embodiment, each R⁷ is independently selected from -Me, —ⁱPr and —F. In an embodiment, each R⁷ is independently selected from -Me and —F. In an embodiment, R⁷ is -Me. In an embodiment, R⁷ is —F

As generally defined herein, each instance of R is independently hydrogen, —C₁₋₆ alkyl, —C₁₋₆ haloalkyl, —C₁₋₆ heteroalkyl, —C₂₋₆ alkenyl, —C₂₋₆ alkynyl, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, or two R groups attached to the same nitrogen are optionally taken together with nitrogen to which they are attached to form an optionally substituted 4-7 heterocyclyl having 0, 1 or 2 additional heteroatoms independently selected from N, O and S.

In an embodiment, each instance of R is independently hydrogen, —C₁₋₆ alkyl, —C₁₋₆ haloalkyl, —C₁₋₆ heteroalkyl, —C₃₋₇ cycloalkyl and 4-7 membered heterocyclyl containing 1-3 heteroatoms independently selected from N, O and S, or two R groups attached to the same nitrogen are optionally taken together with nitrogen to which they are attached to form a 4-7-membered heterocyclyl having 0, 1 or 2 additional heteroatoms independently selected from N, O and S, substituted with 0, 1, 2 or 3 substituents independently selected from —F, -Me, oxo, —OH— or —OMe.

In an embodiment, each instance of R is independently hydrogen, —C₁₋₆ alkyl, —C₁₋₆ haloalkyl, —C₁₋₆ heteroalkyl or —C₃₋₇ cycloalkyl or two R groups attached to the same nitrogen are optionally taken together with nitrogen to which they are attached to form a 4-7-membered heterocyclyl having 0, 1 or 2 additional heteroatoms independently selected from N, O and S, substituted with 0, 1, 2 or 3 substituents independently selected from —F, -Me, oxo, —OH— or —OMe.

In an embodiment, two R groups attached to the same nitrogen are optionally taken together with nitrogen to which they are attached to form an azetidine, pyrrolidine, piperidine, piperazine, morpholine or azepane, each substituted with 0, 1, 2 or 3 substituents independently selected from —F, -Me, oxo, —OH— or —OMe.

In an embodiment, each instance of R is independently selected from hydrogen and —C₁₋₆ alkyl. In an embodiment, R is H. In an embodiment R is —C₁₋₆ alkyl. In an embodiment, R is selected from H and -Me. In an embodiment, R is -Me.

As generally defined herein, each L¹ is independently selected from a bond and —N(R′), wherein R′ is as defined in any of the embodiments described herein. In an embodiment, L¹ is a bond. In an embodiment, L¹ is —N(R′), wherein R′ is as defined in any of the embodiments described herein. In an embodiment, L¹ is selected from a bond, —NH— and —NMe-. In an embodiment, L¹ is selected from a bond and —NH—. In an embodiment, L¹ is selected from a bond —NMe-. In an embodiment, L¹ is —NH—. In an embodiment, L¹ is —NMe-.

As generally defined herein, each L² independently selected from a bond and —N(R′), wherein R′ is as defined in any of the embodiments described herein.

In an embodiment, L² is a bond. In an embodiment, L² is —N(R′), wherein R′ is as defined in any of the embodiments described herein. In an embodiment, L² is selected from a bond, —NH— and —NMe-. In an embodiment, L² is selected from a bond and —NH—. In an embodiment, L² is selected from a bond —NMe-. In an embodiment, L² is —NH—. In an embodiment, L² is —NMe-.

In an embodiment, L¹ is a bond and L² is a bond or N(R′)—. In an embodiment, L¹ is a bond and L² is a bond or NMe-.

In an embodiment, L¹ is a bond and L² is a bond or —NH—.

As generally defined herein, each R′ is independently selected from H and C₁₋₆ alkyl.

As generally defined herein, n is 0, 1, 2, 3, or 4. In an embodiment, n is 0, 1, 2 or 3. In an embodiment, n is 0, 1 or 2. In an embodiment, n is 0 or 1. In an embodiment, n is 0. In an embodiment, n is 1. In an embodiment, n is 2. In an embodiment, n is 3. In an embodiment, n is 4.

As generally defined herein, r is 0, 1, 2, 3, or 4. In an embodiment, r is 0, 1, 2 or 3. In an embodiment, r is 0, 1 or 2. In an embodiment, r is 0 or 1. In an embodiment, r is 0. In an embodiment, r is 1. In an embodiment, r is 2. In an embodiment, r is 3. In an embodiment, r is 4.

As generally defined herein, s is 0, 1, 2, 3, or 4. In an embodiment, s is 0, 1, 2 or 3. In an embodiment, s is 0, 1 or 2. In an embodiment, s is 0 or 1. In an embodiment, s is 0. In an embodiment, s is 1. In an embodiment, s is 2. In an embodiment, s is 3. In an embodiment, s is 4.

As generally defined herein, m is 0, 1, 2, 3, or 4. In an embodiment, m is 0, 1, 2 or 3. In an embodiment, m is 0, 1 or 2. In an embodiment, m is 0 or 1. In an embodiment, m is 0. In an embodiment, m is 1. In an embodiment, m is 2. In an embodiment, m is 3. In an embodiment, m is 4.

As generally defined herein, p is 0, 1, 2, 3, or 4. In an embodiment, p is 0, 1, 2 or 3. In an embodiment, p is 0, 1 or 2. In an embodiment, p is 0 or 1. In an embodiment, p is 0. In an embodiment, p is 1. In an embodiment, p is 2. In an embodiment, p is 3. In an embodiment, p is 4.

As generally defined herein, q is 0, 1, 2, 3, 4, 5, 6, 7, 8, 9 or 10. In an embodiment, q is 1, 2, 3, 4, 5, 7 or 9. In an embodiment, q is 0. In an embodiment, q is 1. In an embodiment, q is 2. In an embodiment, q is 3. In an embodiment, q is 4. In an embodiment, q is 5. In an embodiment, q is 6. In an embodiment, q is 7. In an embodiment, q is 8. In an embodiment, q is 9. In an embodiment, q is 10.

In an embodiment, provided is a compound of Formula B:

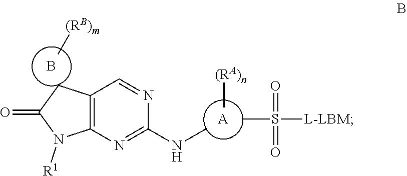

wherein Ring B, R¹, R^{A}, R^{B}, L, LBM, m and n are as defined in any of the embodiments described herein.

In an embodiment, provided is a compound of Formula C:

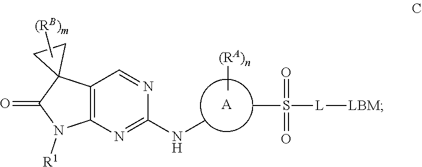

wherein R¹, R^{A}, R^{B}, L, LBM, m and n are as defined in any of the embodiments described herein.

In an embodiment, provided is a compound of Formula D:

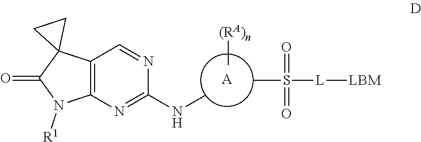

wherein R¹, R^{A}, L, LBM and n are as defined in any of the embodiments described herein.

In an embodiment, provided is a compound of Formula E:

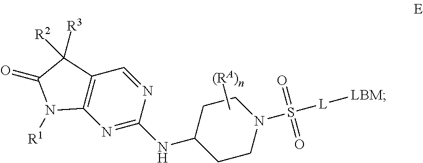

wherein R¹, R², R³, R^{A}, L, LBM and n are as defined in any of the embodiments described herein.

In an embodiment, provided is a compound of Formula F:

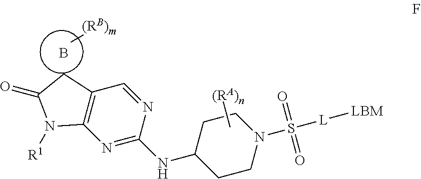

wherein Ring B, R¹, R^{A}, R^{B}, L, LBM, m and n are as defined in any of the embodiments described herein.

In an embodiment, provided is a compound of Formula G:

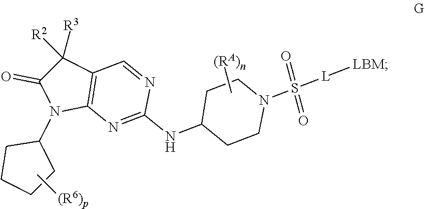

wherein R², R³, R⁶, R^{A}, L, LBM, p and n are as defined in any of the embodiments described herein.

In an embodiment, provided is a compound of Formula H:

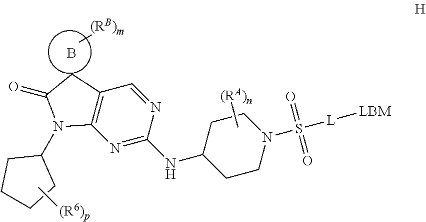

wherein Ring B, R⁶, R^{A}, R^{B}, L, LBM, m, p and n are as defined in any of the embodiments described herein.

In an embodiment, provided is a compound of Formula J:

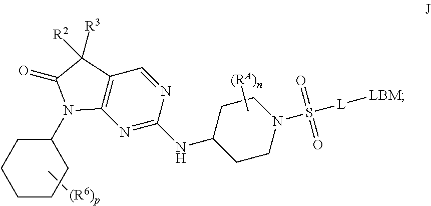

wherein R², R³, R⁶, R^{A}, L, LBM, p and n are as defined in any of the embodiments described herein.

In an embodiment, provided is a compound of Formula K:

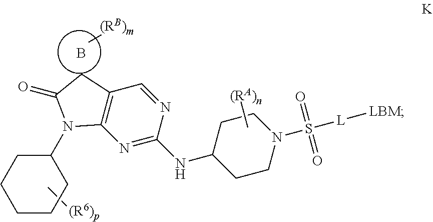

wherein Ring B, R⁶, R^{A}, R^{B}, L, LBM, m, p and n are as defined in any of the embodiments described herein.

In an embodiment, provided is a compound of Formula L:

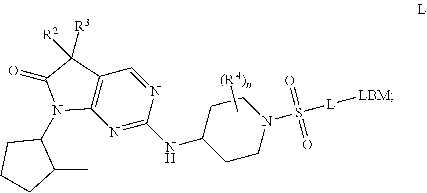

wherein R², R³, R^{A}, L, LBM and n are as defined in any of the embodiments described herein. In an embodiment, provided is a compound of Formula M:

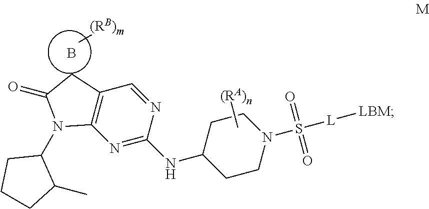

wherein Ring B, R^{A}, R^{B}, L, LBM, m and n are as defined in any of the embodiments described herein.

In an embodiment, provided is a compound of Formula N:

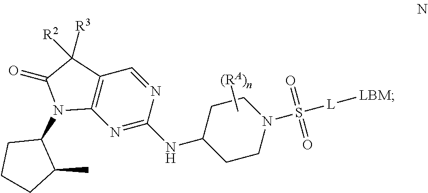

wherein R², R³, R^{A}, L, LBM and n are as defined in any of the embodiments described herein.

In an embodiment, provided is a compound of Formula 0:

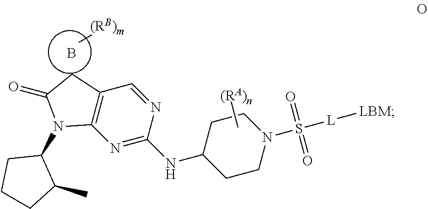

wherein Ring B, R^{A}, R^{B}, L, LBM, m and n are as defined in any of the embodiments described herein.

In an embodiment, provided is a compound of Formula I-A:

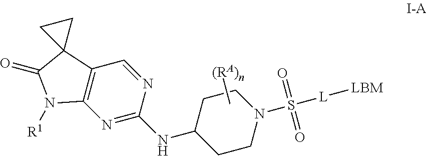

wherein each R¹, R^{A}, L, LBM and n are as defined in any of the embodiments described herein.

In an embodiment, provided is a compound of Formula I-B:

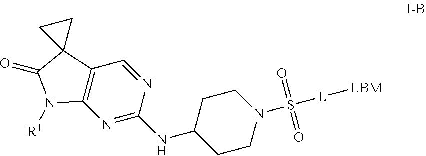

wherein each R¹, L and LBM are as defined in any of the embodiments described herein.

In an embodiment, provided is a compound of Formula I-C:

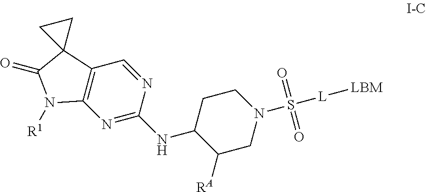

wherein R¹, L and LBM are as defined in any of the embodiments described herein.

In an embodiment, provided is a compound of Formula I-1-A:

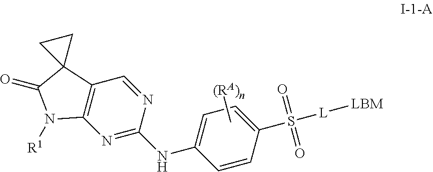

wherein each R¹, R^{A}, L, LBM and n are as defined in any of the embodiments described herein.

In an embodiment, provided is a compound of Formula I-B:

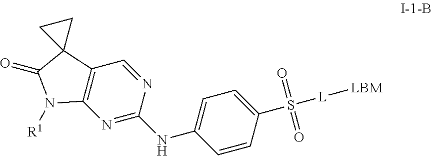

wherein each R¹, L and LBM are as defined in any of the embodiments described herein.

In an embodiment, provided is a compound of Formula II:

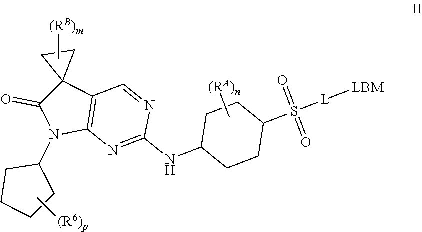

wherein each R⁶, R^{A}, R^{B}, L, LBM, m, n and p are as defined in any of the embodiments described herein.

In an embodiment, provided is a compound of Formula II-A:

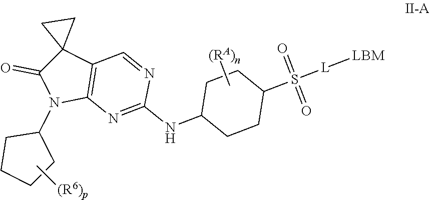

wherein each R⁶, R^{A}, L, LBM, n and p are as defined in any of the embodiments described herein.

In an embodiment, provided is a compound of Formula II-B:

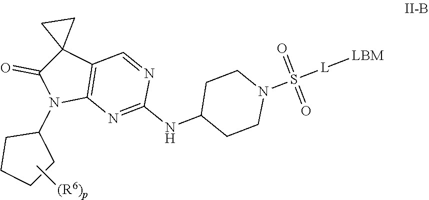

wherein each R⁶, L, LBM and p are as defined in any of the embodiments described herein.

In an embodiment, provided is a compound of Formula II-C:

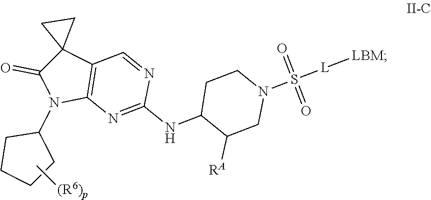

wherein R⁶, R^{A}, p, L and LBM are as defined in any of the embodiments described herein.

In an embodiment, provided is a compound of Formula II-1:

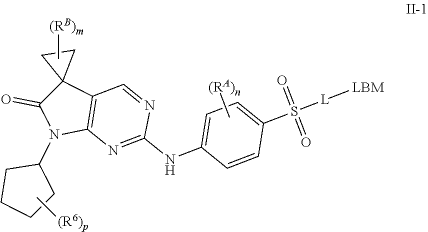

wherein each R⁶, R^{A}, R^{B}, L, LBM, m, n and p are as defined in any of the embodiments described herein.

In an embodiment, provided is a compound of Formula II-1-A:

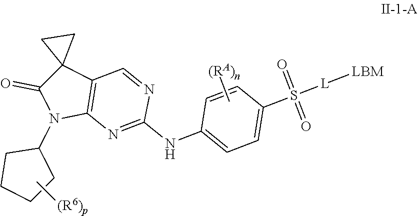

wherein each R⁶, R^{A}, L, LBM, n and p are as defined in any of the embodiments described herein.

In an embodiment, provided is a compound of Formula II-1-B:

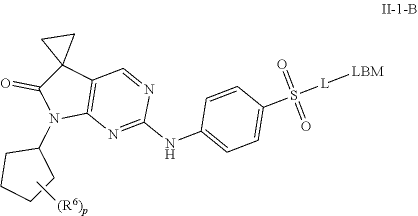

wherein each R⁶, L, LBM and p are as defined in any of the embodiments described herein.

In an embodiment, provided is a compound of Formula III:

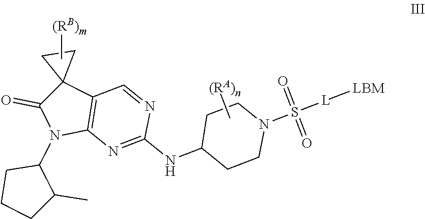

wherein each R^{A}, R^{B}, L, LBM, m and n are as defined in any of the embodiments described herein.

In an embodiment, provided is a compound of Formula III-A:

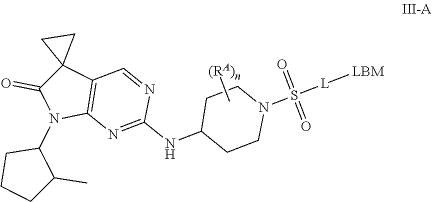

wherein each R^{A}, L, LBM and n are as defined in any of the embodiments described herein.

In an embodiment, provided is a compound of Formula III-B:

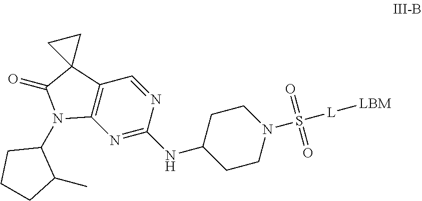

wherein each L and LBM are as defined in any of the embodiments described herein.

In an embodiment, provided is a compound of Formula III-C:

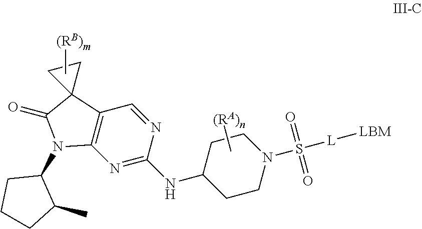

wherein each R^{A}, R^{B}, L, LBM, m and n are as defined in any of the embodiments described herein.

In an embodiment, provided is a compound of Formula III-D:

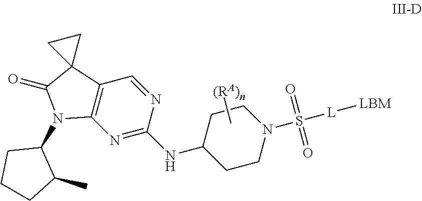

wherein each R^{A}, L, LBM and n are as defined in any of the embodiments described herein.

In an embodiment, provided is a compound of Formula III-E:

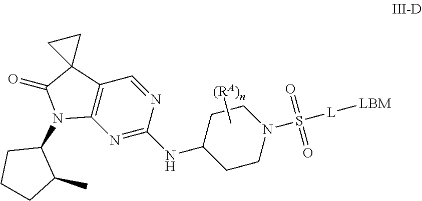

wherein each L and LBM are as defined in any of the embodiments described herein.

In an embodiment, compound is of Formula III-F:

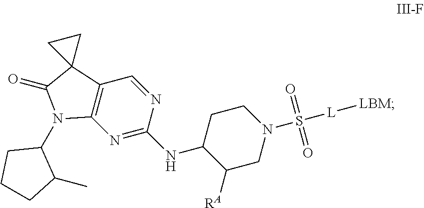

wherein each R^{A}, L and LBM are as defined in any of the embodiments described herein.

In an embodiment, provided is a compound of Formula III-G:

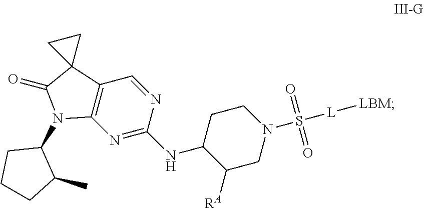

wherein each R^{A}, L and LBM are as defined in any of the embodiments described herein.

In an embodiment, provided is a compound of Formula III-1:

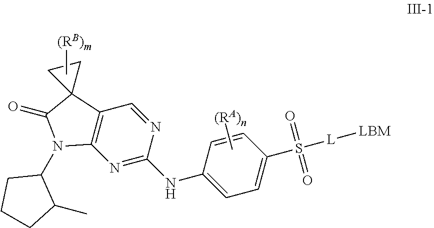

wherein each R^{A}, R^{B}, L, LBM, m and n are as defined in any of the embodiments described herein.

In an embodiment, provided is a compound of Formula III-1-A:

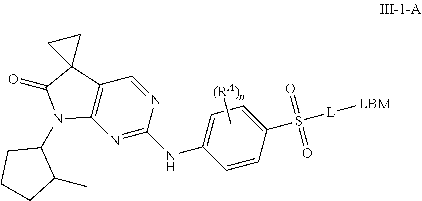

wherein each R^{A}, L, LBM and n are as defined in any of the embodiments described herein.

In an embodiment, provided is a compound of Formula III-1-B:

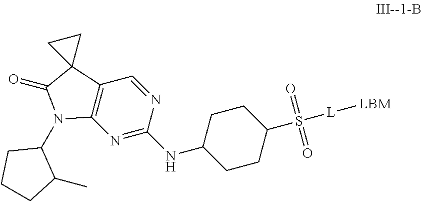

wherein each L and LBM are as defined in any of the embodiments described herein.

In an embodiment, provided is a compound of Formula III-1-C:

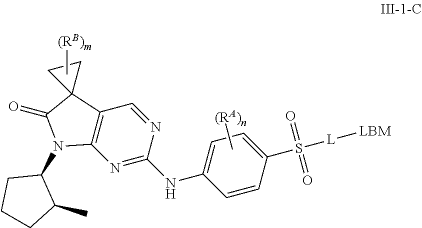

wherein each R^{A}, R^{B}, L, LBM, m and n are as defined in any of the embodiments described herein.

In an embodiment, provided is a compound of Formula III-1-D:

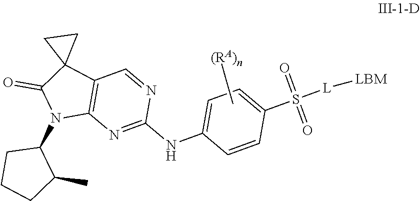

wherein each R^{A}, L, LBM and n are as defined in any of the embodiments described herein.

In an embodiment, provided is a compound of Formula III-1-E:

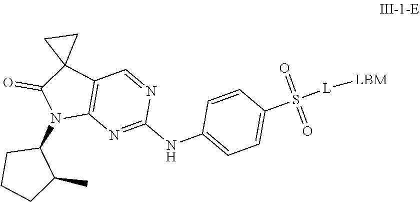

wherein each L and LBM are as defined in any of the embodiments described herein.

In an embodiment, provided is a compound of Formula IV:

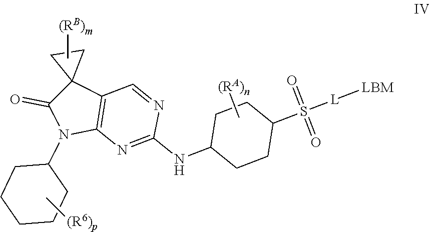

wherein each R^{B}, R⁶, R^{A}, L, LBM, m, n and p are as defined in any of the embodiments described herein.

In an embodiment, provided is a compound of Formula IV-A:

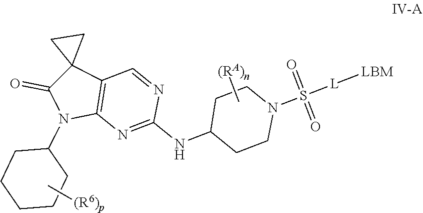

wherein each R⁶, R^{A}, L, LBM, n and p are as defined in any of the embodiments described herein.

In an embodiment, provided is a compound of Formula IV-B:

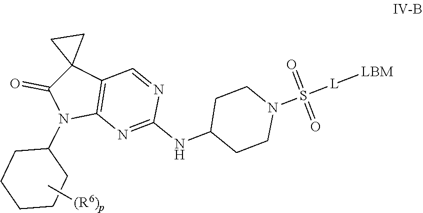

wherein each R⁶, L, LBM and p are as defined in any of the embodiments described herein.

In an embodiment, provided is a compound of Formula IV-C:

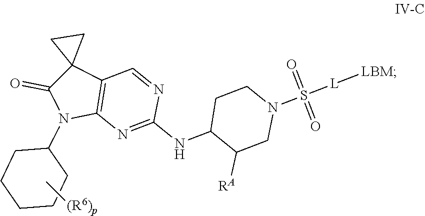

wherein R⁶, R^{A}, p, L and LBM are as defined in any of the embodiments described herein.

In an embodiment, provided is a compound of Formula IV-1:

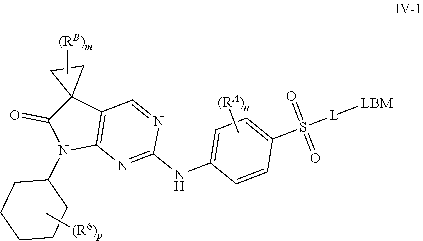

wherein each R^{B}, R⁶, R^{A}, L, LBM, m, n and p are as defined in any of the embodiments described herein.

In an embodiment, provided is a compound of Formula IV-1-A:

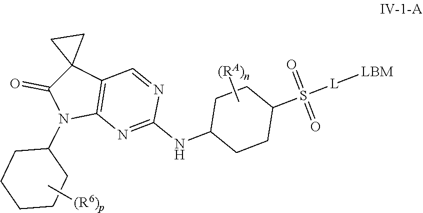

wherein each R⁶, R^{A}, L, LBM, n and p are as defined in any of the embodiments described herein.

In an embodiment, provided is a compound of Formula IV-1-B:

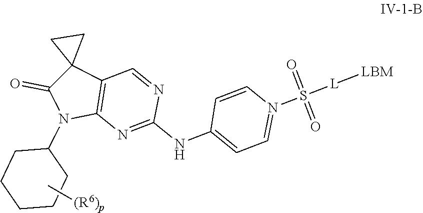

wherein each R⁶, L, LBM and p are as defined in any of the embodiments described herein.

In an embodiment, provided is a compound of Formula V:

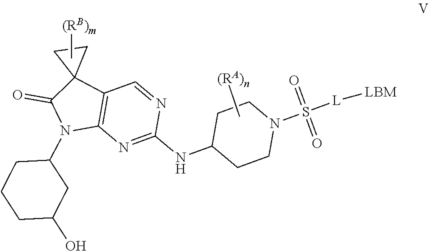

wherein each R^{A}, R^{B}, L, LBM, m and n are as defined in any of the embodiments described herein.

In an embodiment, provided is a compound of Formula V-A:

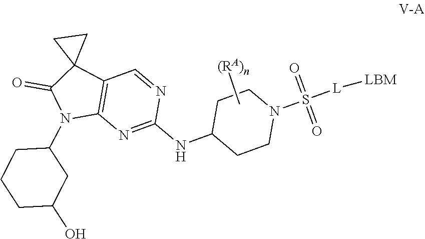

wherein each R^{A}, L, LBM and n are as defined in any of the embodiments described herein.

In an embodiment, provided is a compound of Formula V-B:

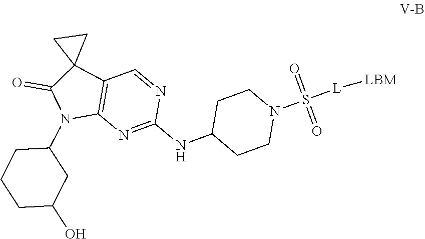

wherein each L and LBM are as defined in any of the embodiments described herein.

In an embodiment, provided is a compound of Formula V-C:

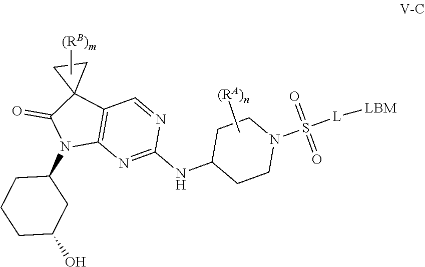

wherein each R^{A}, R^{B}, L, LBM, m and n are as defined in any of the embodiments described herein.

In an embodiment, provided is a compound of Formula V-D:

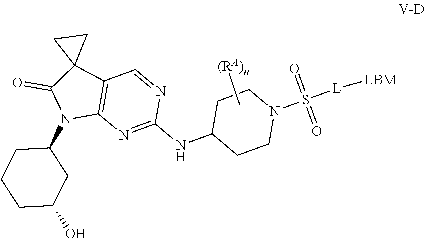

wherein each R^{A}, L, LBM and n are as defined in any of the embodiments described herein.

In an embodiment, provided is a compound of Formula V-E:

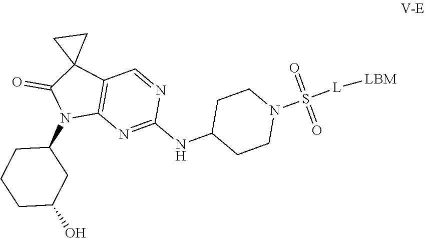

wherein each L and LBM are as defined in any of the embodiments described herein.

In an embodiment, provided is a compound of Formula V-F:

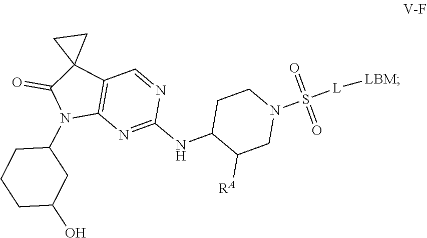

wherein each R^{A}, L and LBM are as defined in any of the embodiments described herein.

In an embodiment, provided is a compound of Formula V-G:

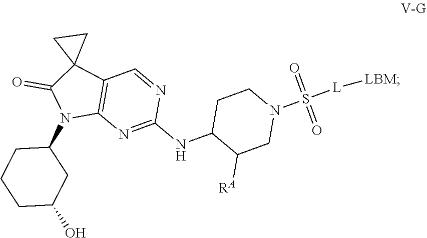

wherein each R^{A}, L and LBM are as defined in any of the embodiments described herein.

In an embodiment, provided is a compound of Formula V-1:

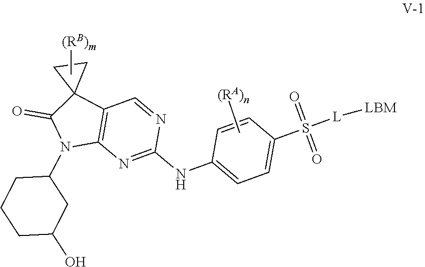

wherein each R^{A}, R^{B}, L, LBM, m and n are as defined in any of the embodiments described herein.

In an embodiment, provided is a compound of Formula V-1-A:

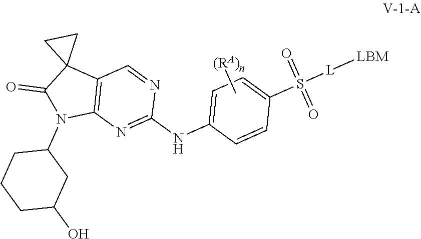

wherein each R^{A}, L, LBM and n are as defined in any of the embodiments described herein.

In an embodiment, provided is a compound of Formula V-1-B:

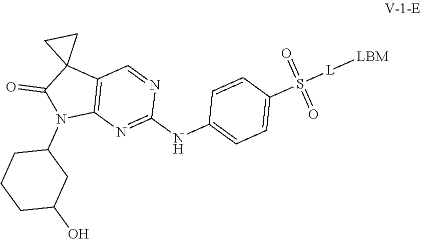

wherein each L and LBM are as defined in any of the embodiments described herein.

In an embodiment, provided is a compound of Formula V-1-C:

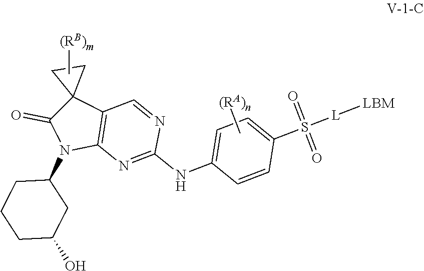

wherein each R^{A}, R^{B}, L, LBM, m and n are as defined in any of the embodiments described herein.

In an embodiment, provided is a compound of Formula V-1-D:

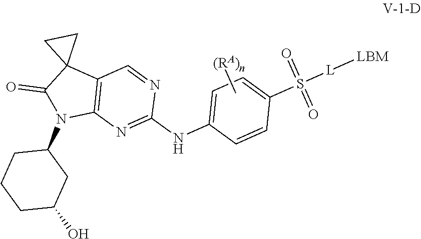

wherein each R^{A}, L, LBM and n are as defined in any of the embodiments described herein.

In an embodiment, provided is a compound of Formula V-1-E:

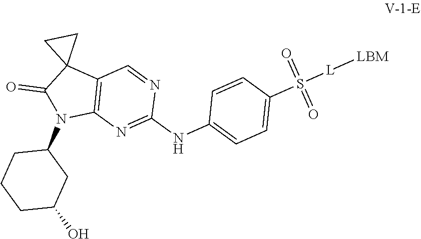

wherein each L and LBM are as defined in any of the embodiments described herein.

In an embodiment of a compound of Formula A, Formula I or Formula I-1, the compound is selected from the compounds disclosed in Table 1, or a pharmaceutically acceptable salt thereof, or elsewhere in the specification and figures.

In an embodiment, provided herein is a composition comprising a compound described herein and a pharmaceutically acceptable excipient.

In an embodiment, the compound is a compound identified in Table 1 below or a pharmaceutically acceptable salt thereof.

Unless otherwise indicated, the absolute stereochemistry of all chiral atoms is as depicted. Compounds marked with (or) or (rel) in Table 1 and the Examples section are single enantiomers wherein the absolute stereochemistry was arbitrarily assigned (e.g., based on chiral SFC elution as described in the Examples section). Compounds marked with (and) or (rac) are mixtures of enantiomers wherein the relative stereochemistry is as shown. Compounds that have a stereogenic center where the configuration is not indicated in the structure as depicted and that have no designation in the stereochemistry column of Table 1 are mixtures of enantiomers at that center. Compounds that have a stereogenic center where the configuration is indicated in the structure as depicted and have no designation in the stereochemistry column of Table 1 or that are marked with (abs) are single enantiomers wherein the absolute stereochemistry is as indicated.

A person of skill in the art would be able to separate racemic compounds into the respective enantiomers using methods known in the art, such as chiral chromatography, chiral recrystallization and the like. References to compounds that are racemic mixtures are meant to also include the individual enantiomers contained in the mixture.

TABLE 1
Exemplary compounds

| Structure | Nr |
|---|---|
| 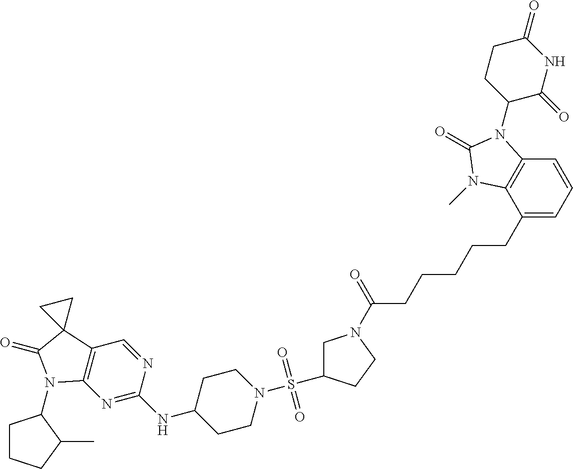 [SMILES: CC1CCCC1N1C(=O)C2(CC2)c2cnc(NC3CCN(S(=O)(=O)C4CCN(C(=O)CCCCCc5cccc6c5n(C)c(=O)n6C5CCC(=O)NC5=O)C4)CC3)nc21] | 1 |
| 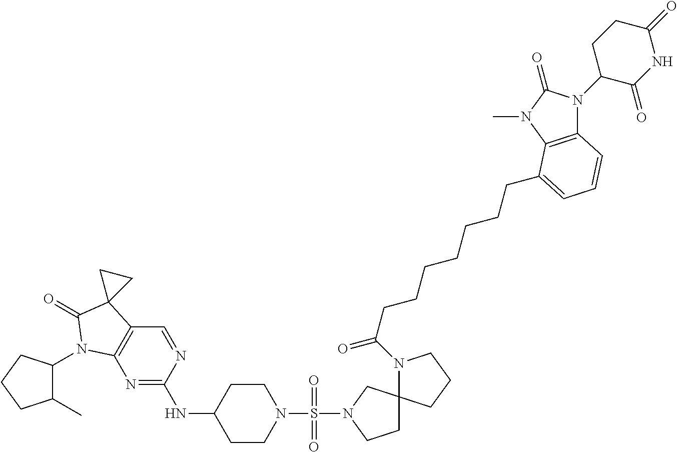 [SMILES: CC1CCCC1N1C(=O)C2(CC2)c2cnc(NC3CCN(S(=O)(=O)N4CCC5(CCCN5C(=O)CCCCCCCc5cccc6c5n(C)c(=O)n6C5CCC(=O)NC5=O)C4)CC3)nc21] | 2 |
| 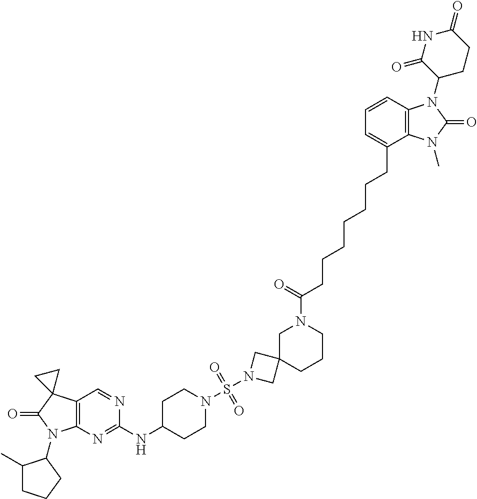 [SMILES: CC1CCCC1N1C(=O)C2(CC2)c2cnc(NC3CCN(S(=O)(=O)N4CC5(CCCN(C(=O)CCCCCCCc6cccc7c6n(C)c(=O)n7C6CCC(=O)NC6=O)C5)C4)CC3)nc21] | 3 |
| 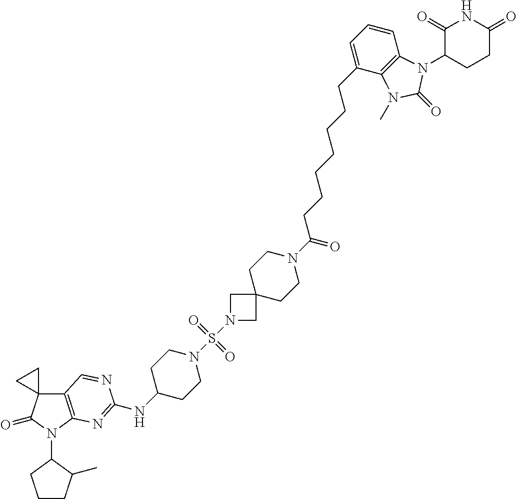 [SMILES: CC1CCCC1N1C(=O)C2(CC2)c2cnc(NC3CCN(S(=O)(=O)N4CC5(CCN(C(=O)CCCCCCCc6cccc7c6n(C)c(=O)n7C6CCC(=O)NC6=O)CC5)C4)CC3)nc21] | 4 |
| 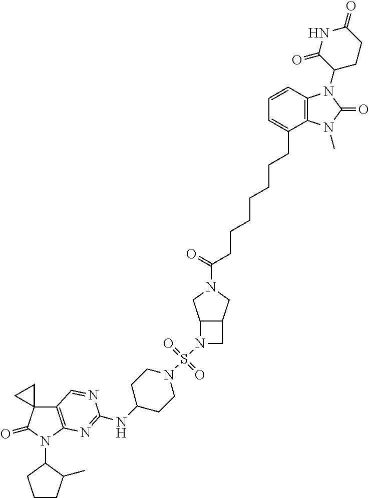 [SMILES: CC1CCCC1N1C(=O)C2(CC2)c2cnc(NC3CCN(S(=O)(=O)N4CC5CN(C(=O)CCCCCCCc6cccc7c6n(C)c(=O)n7C6CCC(=O)NC6=O)CC54)CC3)nc21] | 5 |
| 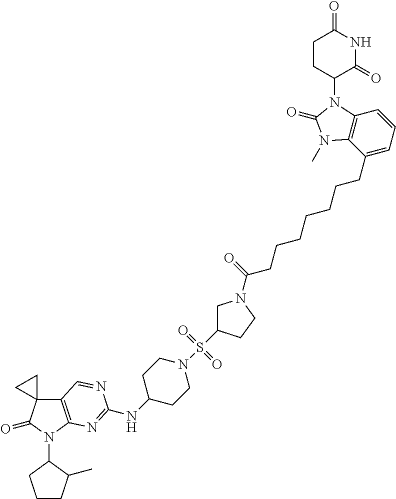 [SMILES: CC1CCCC1N1C(=O)C2(CC2)c2cnc(NC3CCN(S(=O)(=O)C4CCN(C(=O)CCCCCCCc5cccc6c5n(C)c(=O)n6C5CCC(=O)NC5=O)C4)CC3)nc21] | 6 |
| 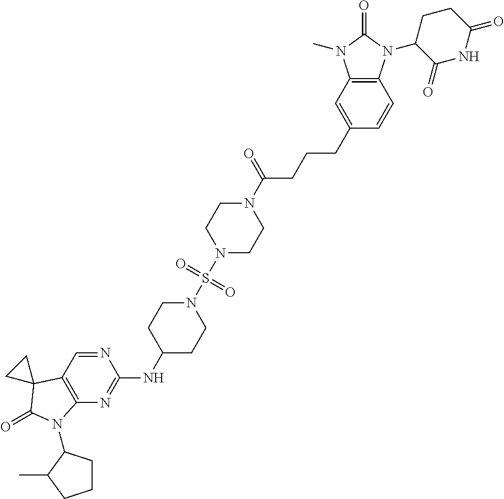 [SMILES: CC1CCCC1N1C(=O)C2(CC2)c2cnc(NC3CCN(S(=O)(=O)N4CCN(C(=O)CCCc5ccc6c(c5)n(C)c(=O)n6C5CCC(=O)NC5=O)CC4)CC3)nc21] | 7 |
| 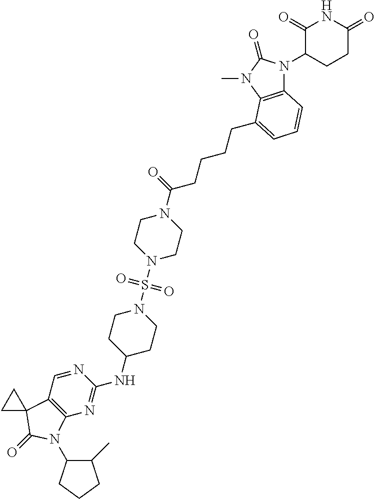 [SMILES: CC1CCCC1N1C(=O)C2(CC2)c2cnc(NC3CCN(S(=O)(=O)N4CCN(C(=O)CCCCc5cccc6c5n(C)c(=O)n6C5CCC(=O)NC5=O)CC4)CC3)nc21] | 8 |
| 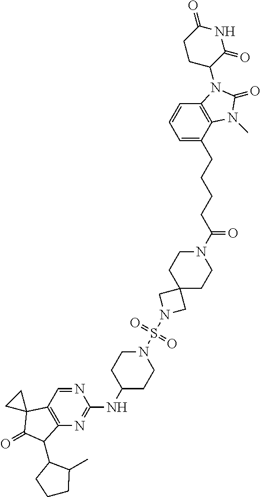 [SMILES: CC1CCCC1C1C(=O)C2(CC2)c2cnc(NC3CCN(S(=O)(=O)N4CC5(CCN(C(=O)CCCCc6cccc7c6n(C)c(=O)n7C6CCC(=O)NC6=O)CC5)C4)CC3)nc21] | 9 |
| 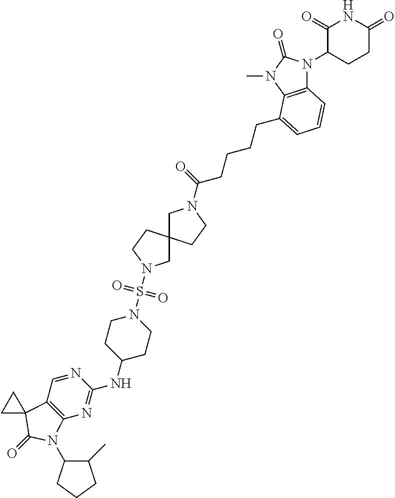 [SMILES: CC1CCCC1N1C(=O)C2(CC2)c2cnc(NC3CCN(S(=O)(=O)N4CCC5(CCN(C(=O)CCCCc6cccc7c6n(C)c(=O)n7C6CCC(=O)NC6=O)C5)C4)CC3)nc21] | 10 |
| 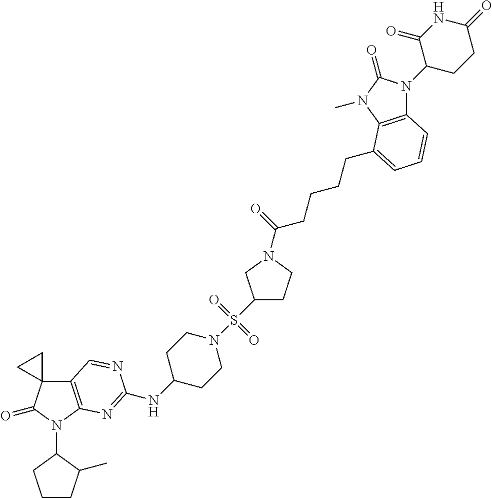 [SMILES: CC1CCCC1N1C(=O)C2(CC2)c2cnc(NC3CCN(S(=O)(=O)C4CCN(C(=O)CCCCc5cccc6c5n(C)c(=O)n6C5CCC(=O)NC5=O)C4)CC3)nc21] | 11 |
| 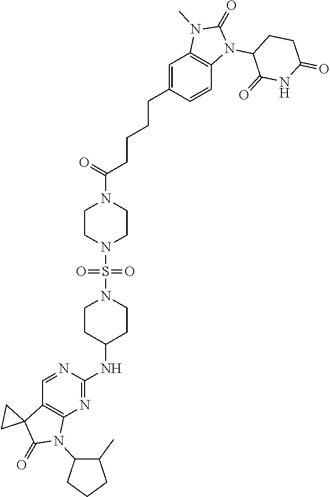 [SMILES: CC1CCCC1N1C(=O)C2(CC2)c2cnc(NC3CCN(S(=O)(=O)N4CCN(C(=O)CCCCc5ccc6c(c5)n(C)c(=O)n6C5CCC(=O)NC5=O)CC4)CC3)nc21] | 12 |
| 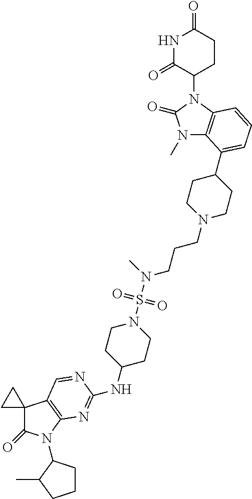 [SMILES: CC1CCCC1N1C(=O)C2(CC2)c2cnc(NC3CCN(S(=O)(=O)N(C)CCCN4CCC(c5cccc6c5n(C)c(=O)n6C5CCC(=O)NC5=O)CC4)CC3)nc21] | 13 |
| 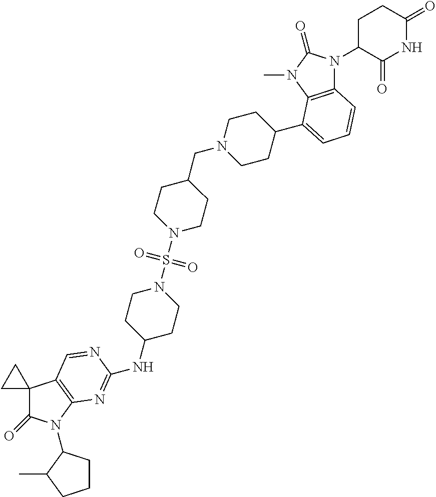 [SMILES: CC1CCCC1N1C(=O)C2(CC2)c2cnc(NC3CCN(S(=O)(=O)N4CCC(CN5CCC(c6cccc7c6n(C)c(=O)n7C6CCC(=O)NC6=O)CC5)CC4)CC3)nc21] | 14 |
| 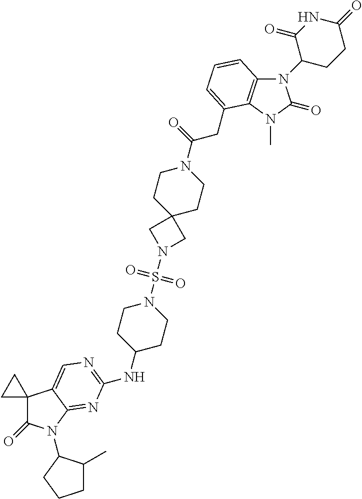 [SMILES: CC1CCCC1N1C(=O)C2(CC2)c2cnc(NC3CCN(S(=O)(=O)N4CC5(CCN(C(=O)Cc6cccc7c6n(C)c(=O)n7C6CCC(=O)NC6=O)CC5)C4)CC3)nc21] | 15 |
| 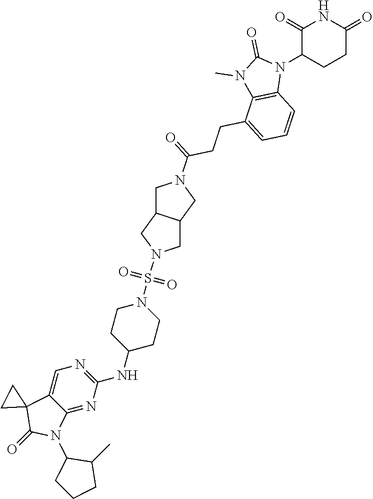 [SMILES: CC1CCCC1N1C(=O)C2(CC2)c2cnc(NC3CCN(S(=O)(=O)N4CC5CN(C(=O)CCc6cccc7c6n(C)c(=O)n7C6CCC(=O)NC6=O)CC5C4)CC3)nc21] | 16 |
| 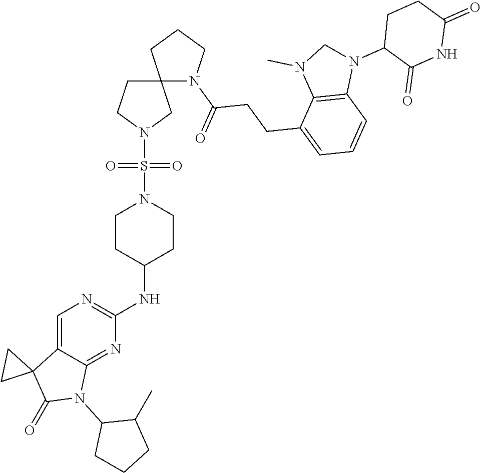 [SMILES: CC1CCCC1N1C(=O)C2(CC2)c2cnc(NC3CCN(S(=O)(=O)N4CCC5(CCCN5C(=O)CCc5cccc6c5N(C)CN6C5CCC(=O)NC5=O)C4)CC3)nc21] | 17 |
| 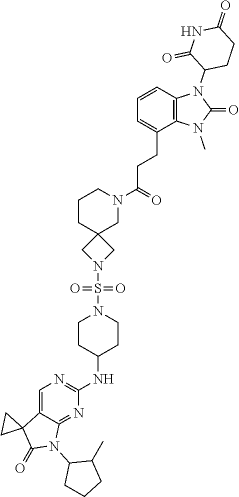 [SMILES: CC1CCCC1N1C(=O)C2(CC2)c2cnc(NC3CCN(S(=O)(=O)N4CC5(CCCN(C(=O)CCc6cccc7c6n(C)c(=O)n7C6CCC(=O)NC6=O)C5)C4)CC3)nc21] | 18 |
| 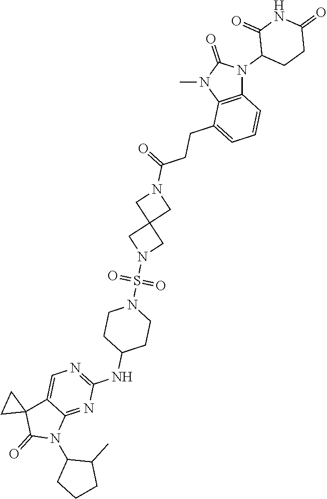 [SMILES: CC1CCCC1N1C(=O)C2(CC2)c2cnc(NC3CCN(S(=O)(=O)N4CC5(CN(C(=O)CCc6cccc7c6n(C)c(=O)n7C6CCC(=O)NC6=O)C5)C4)CC3)nc21] | 19 |
| 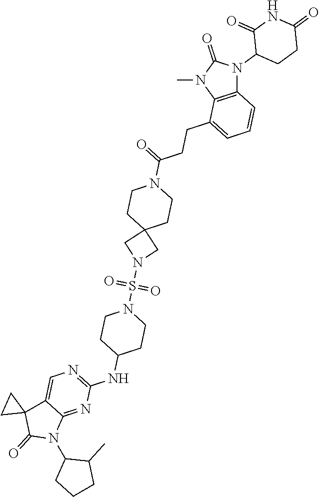 [SMILES: CC1CCCC1N1C(=O)C2(CC2)c2cnc(NC3CCN(S(=O)(=O)N4CC5(CCN(C(=O)CCc6cccc7c6n(C)c(=O)n7C6CCC(=O)NC6=O)CC5)C4)CC3)nc21] | 20 |
| 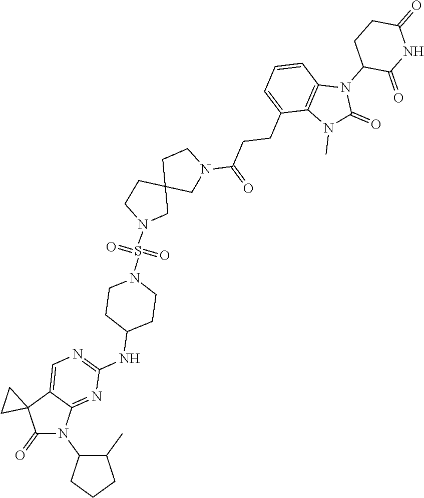 [SMILES: CC1CCCC1N1C(=O)C2(CC2)c2cnc(NC3CCN(S(=O)(=O)N4CCC5(CCN(C(=O)CCc6cccc7c6n(C)c(=O)n7C6CCC(=O)NC6=O)C5)C4)CC3)nc21] | 21 |
| 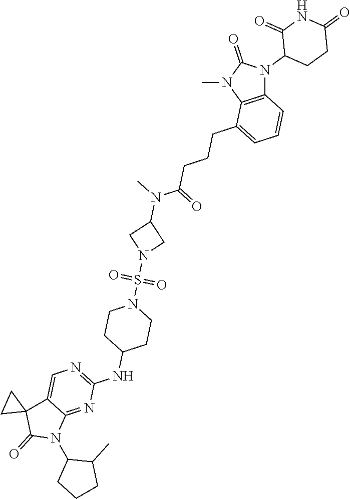 [SMILES: CC1CCCC1N1C(=O)C2(CC2)c2cnc(NC3CCN(S(=O)(=O)N4CC(N(C)C(=O)CCCc5cccc6c5n(C)c(=O)n6C5CCC(=O)NC5=O)C4)CC3)nc21] | 22 |
| 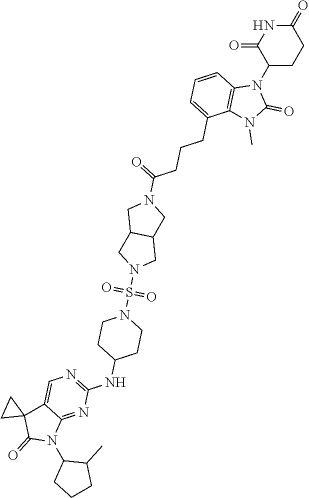 [SMILES: CC1CCCC1N1C(=O)C2(CC2)c2cnc(NC3CCN(S(=O)(=O)N4CC5CN(C(=O)CCCc6cccc7c6n(C)c(=O)n7C6CCC(=O)NC6=O)CC5C4)CC3)nc21] | 23 |
| 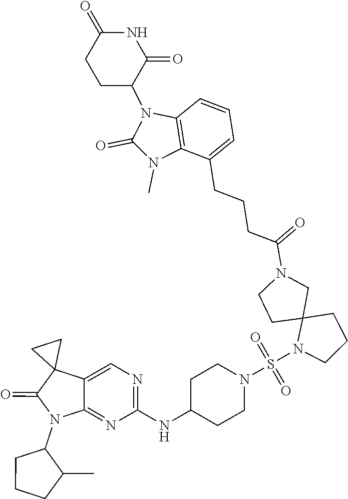 [SMILES: CC1CCCC1N1C(=O)C2(CC2)c2cnc(NC3CCN(S(=O)(=O)N4CCCC45CCN(C(=O)CCCc4cccc6c4n(C)c(=O)n6C4CCC(=O)NC4=O)C5)CC3)nc21] | 24 |
| 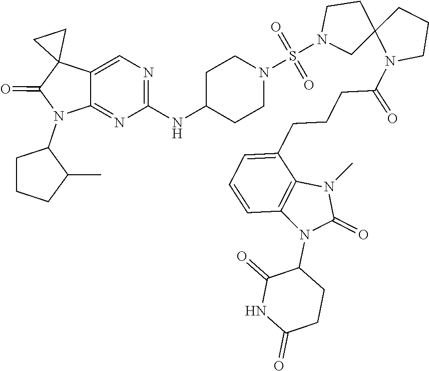 [SMILES: CC1CCCC1N1C(=O)C2(CC2)c2cnc(NC3CCN(S(=O)(=O)N4CCC5(CCCN5C(=O)CCCc5cccc6c5n(C)c(=O)n6C5CCC(=O)NC5=O)C4)CC3)nc21] | 25 |
| 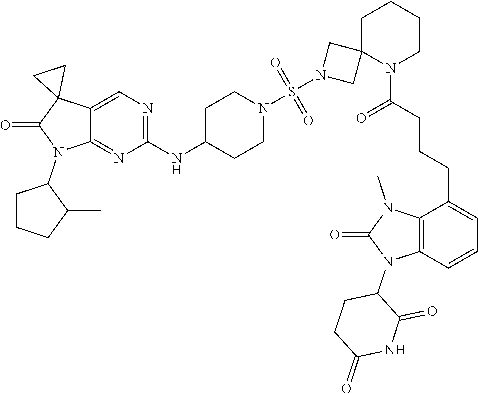 [SMILES: CC1CCCC1N1C(=O)C2(CC2)c2cnc(NC3CCN(S(=O)(=O)N4CC5(CCCCN5C(=O)CCCc5cccc6c5n(C)c(=O)n6C5CCC(=O)NC5=O)C4)CC3)nc21] | 26 |
| 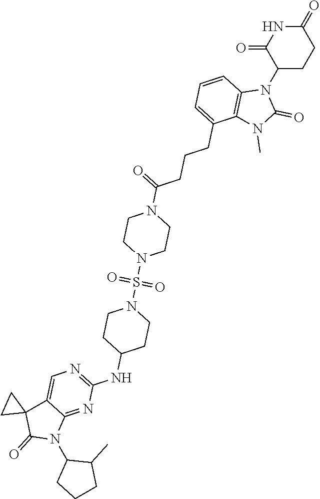 [SMILES: CC1CCCC1N1C(=O)C2(CC2)c2cnc(NC3CCN(S(=O)(=O)N4CCN(C(=O)CCCc5cccc6c5n(C)c(=O)n6C5CCC(=O)NC5=O)CC4)CC3)nc21] | 27 |
| 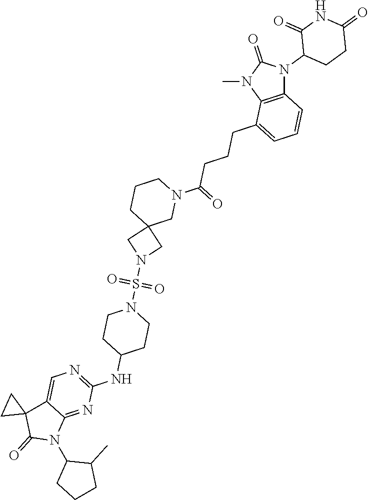 [SMILES: CC1CCCC1N1C(=O)C2(CC2)c2cnc(NC3CCN(S(=O)(=O)N4CC5(CCCN(C(=O)CCCc6cccc7c6n(C)c(=O)n7C6CCC(=O)NC6=O)C5)C4)CC3)nc21] | 28 |
| 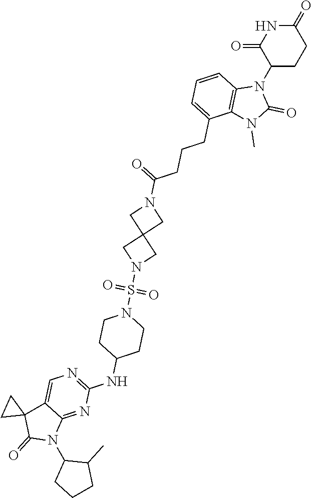 [SMILES: CC1CCCC1N1C(=O)C2(CC2)c2cnc(NC3CCN(S(=O)(=O)N4CC5(CN(C(=O)CCCc6cccc7c6n(C)c(=O)n7C6CCC(=O)NC6=O)C5)C4)CC3)nc21] | 29 |
| 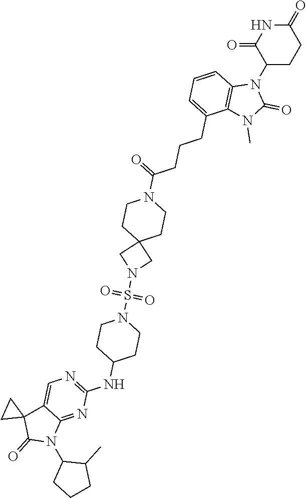 [SMILES: CC1CCCC1N1C(=O)C2(CC2)c2cnc(NC3CCN(S(=O)(=O)N4CC5(CCN(C(=O)CCCc6cccc7c6n(C)c(=O)n7C6CCC(=O)NC6=O)CC5)C4)CC3)nc21] | 30 |
| 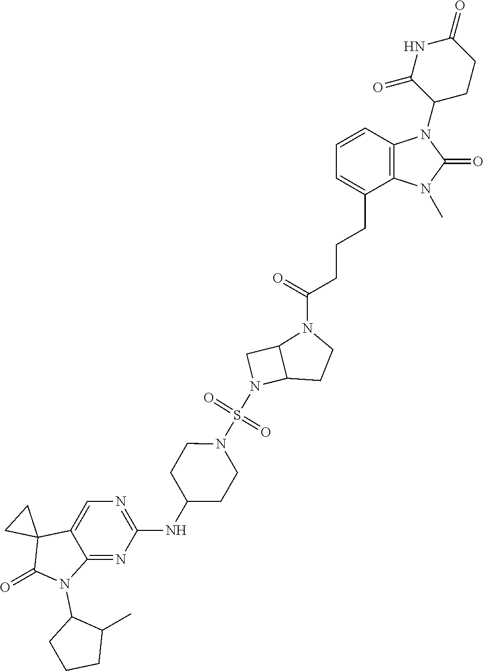 [SMILES: CC1CCCC1N1C(=O)C2(CC2)c2cnc(NC3CCN(S(=O)(=O)N4CC5C4CCN5C(=O)CCCc4cccc5c4n(C)c(=O)n5C4CCC(=O)NC4=O)CC3)nc21] | 31 |
| 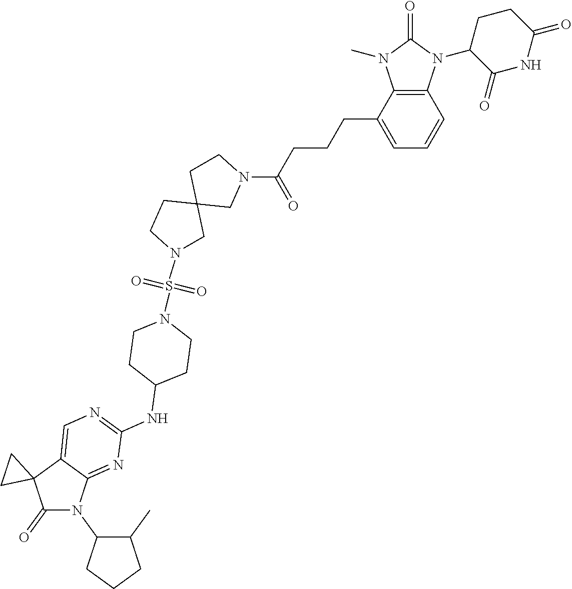 [SMILES: CC1CCCC1N1C(=O)C2(CC2)c2cnc(NC3CCN(S(=O)(=O)N4CCC5(CCN(C(=O)CCCc6cccc7c6n(C)c(=O)n7C6CCC(=O)NC6=O)C5)C4)CC3)nc21] | 32 |
| 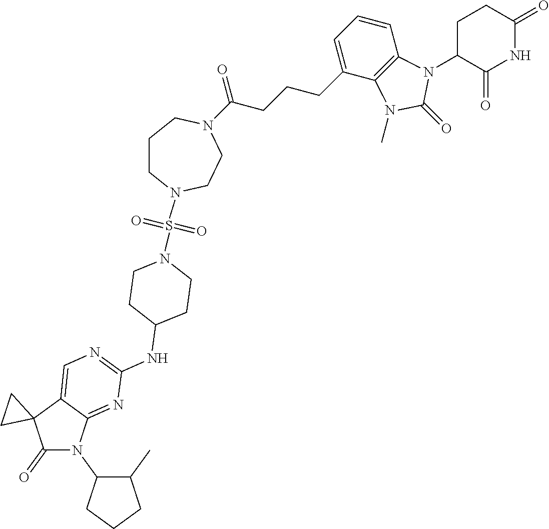 [SMILES: CC1CCCC1N1C(=O)C2(CC2)c2cnc(NC3CCN(S(=O)(=O)N4CCCN(C(=O)CCCc5cccc6c5n(C)c(=O)n6C5CCC(=O)NC5=O)CC4)CC3)nc21] | 33 |
| 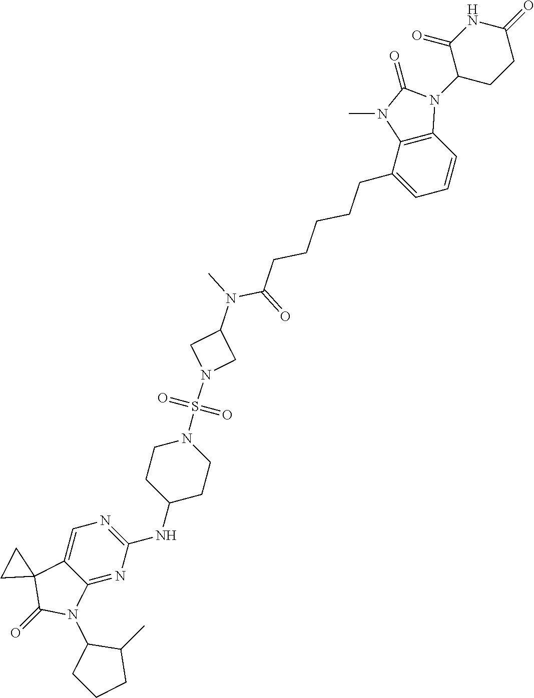 [SMILES: CC1CCCC1N1C(=O)C2(CC2)c2cnc(NC3CCN(S(=O)(=O)N4CC(N(C)C(=O)CCCCCc5cccc6c5n(C)c(=O)n6C5CCC(=O)NC5=O)C4)CC3)nc21] | 34 |
| 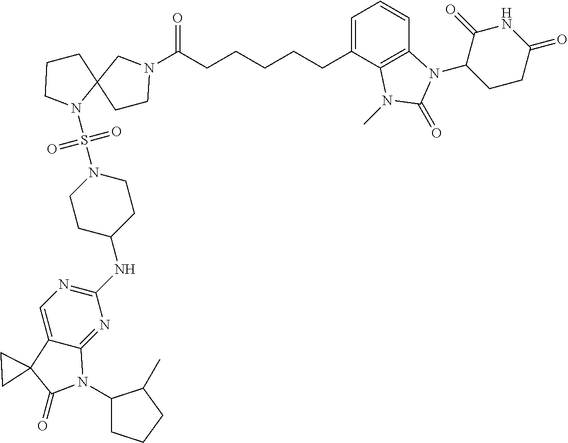 [SMILES: CC1CCCC1N1C(=O)C2(CC2)c2cnc(NC3CCN(S(=O)(=O)N4CCCC45CCN(C(=O)CCCCCc4cccc6c4n(C)c(=O)n6C4CCC(=O)NC4=O)C5)CC3)nc21] | 35 |
| 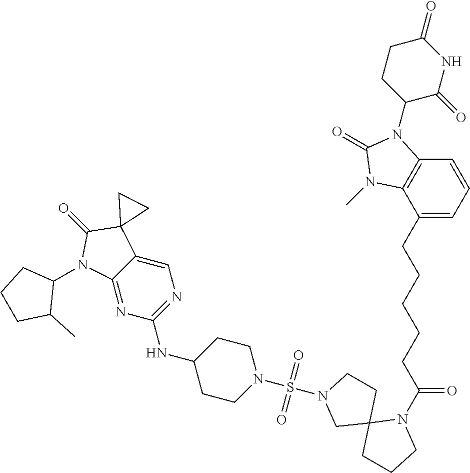 [SMILES: CC1CCCC1N1C(=O)C2(CC2)c2cnc(NC3CCN(S(=O)(=O)N4CCC5(CCCN5C(=O)CCCCCc5cccc6c5n(C)c(=O)n6C5CCC(=O)NC5=O)C4)CC3)nc21] | 36 |
| 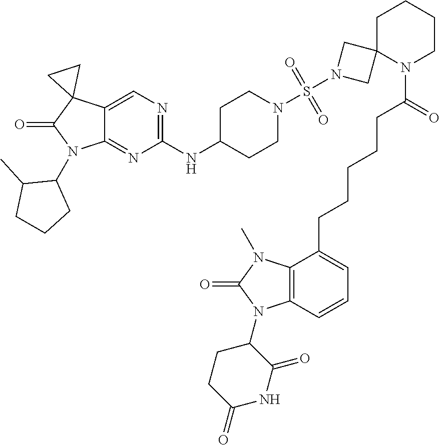 [SMILES: CC1CCCC1N1C(=O)C2(CC2)c2cnc(NC3CCN(S(=O)(=O)N4CC5(CCCCN5C(=O)CCCCCc5cccc6c5n(C)c(=O)n6C5CCC(=O)NC5=O)C4)CC3)nc21] | 37 |
| 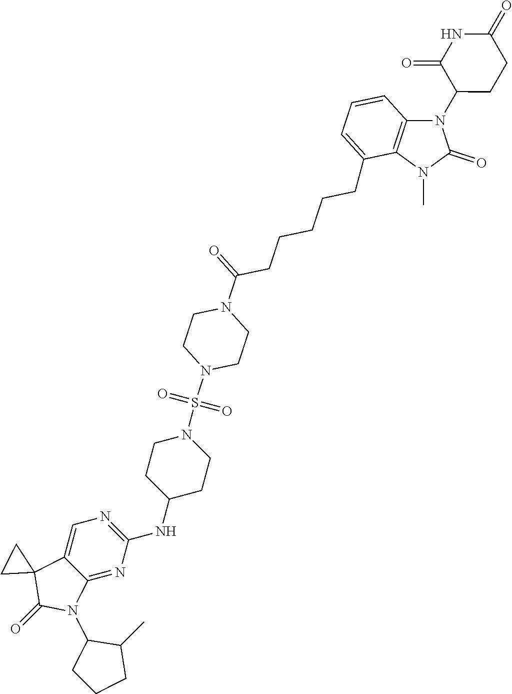 [SMILES: CC1CCCC1N1C(=O)C2(CC2)c2cnc(NC3CCN(S(=O)(=O)N4CCN(C(=O)CCCCCc5cccc6c5n(C)c(=O)n6C5CCC(=O)NC5=O)CC4)CC3)nc21] | 38 |
| 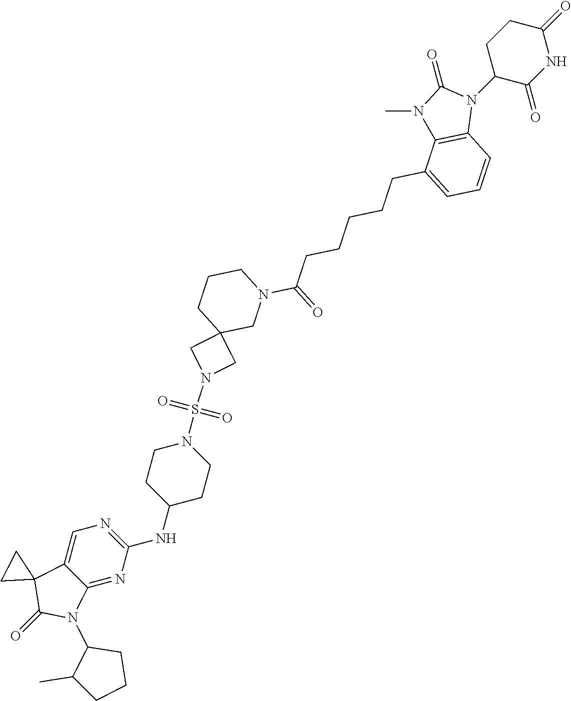 [SMILES: CC1CCCC1N1C(=O)C2(CC2)c2cnc(NC3CCN(S(=O)(=O)N4CC5(CCCN(C(=O)CCCCCc6cccc7c6n(C)c(=O)n7C6CCC(=O)NC6=O)C5)C4)CC3)nc21] | 39 |
| 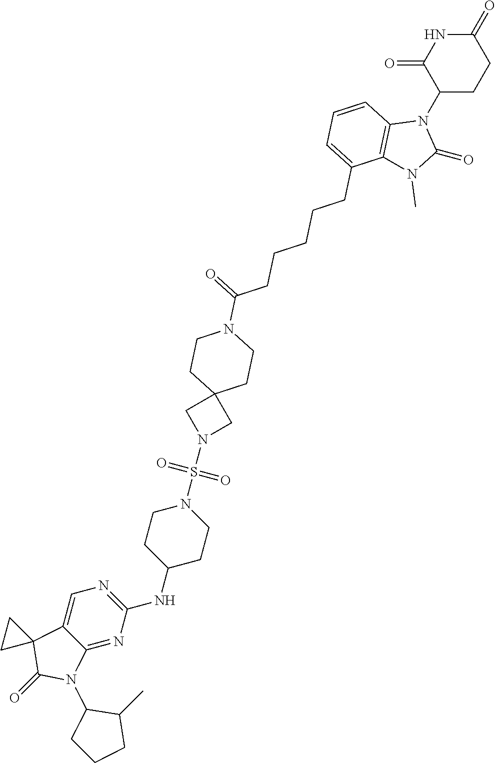 [SMILES: CC1CCCC1N1C(=O)C2(CC2)c2cnc(NC3CCN(S(=O)(=O)N4CC5(CCN(C(=O)CCCCCc6cccc7c6n(C)c(=O)n7C6CCC(=O)NC6=O)CC5)C4)CC3)nc21] | 40 |
| 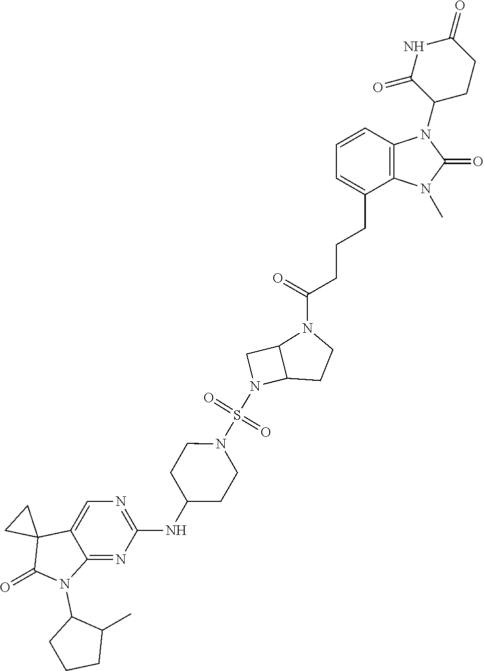 [SMILES: CC1CCCC1N1C(=O)C2(CC2)c2cnc(NC3CCN(S(=O)(=O)N4CC5C4CCN5C(=O)CCCc4cccc5c4n(C)c(=O)n5C4CCC(=O)NC4=O)CC3)nc21] | 41 |
| 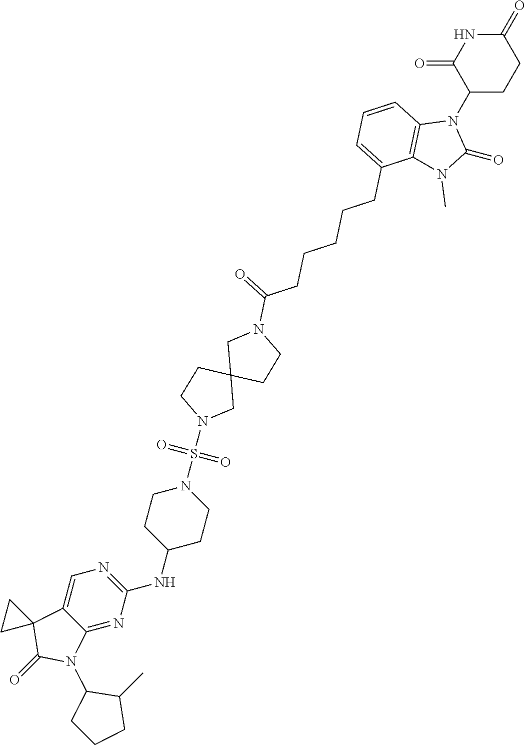 [SMILES: CC1CCCC1N1C(=O)C2(CC2)c2cnc(NC3CCN(S(=O)(=O)N4CCC5(CCN(C(=O)CCCCCc6cccc7c6n(C)c(=O)n7C6CCC(=O)NC6=O)C5)C4)CC3)nc21] | 42 |
| 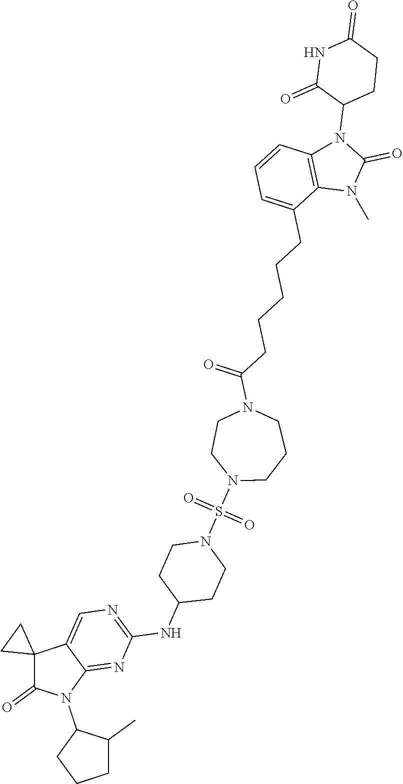 [SMILES: CC1CCCC1N1C(=O)C2(CC2)c2cnc(NC3CCN(S(=O)(=O)N4CCCN(C(=O)CCCCCc5cccc6c5n(C)c(=O)n6C5CCC(=O)NC5=O)CC4)CC3)nc21] | 43 |
| 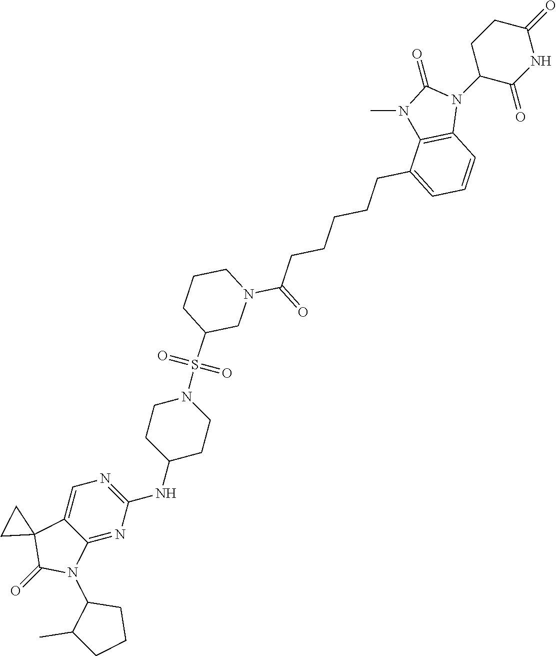 [SMILES: CC1CCCC1N1C(=O)C2(CC2)c2cnc(NC3CCN(S(=O)(=O)C4CCCN(C(=O)CCCCCc5cccc6c5n(C)c(=O)n6C5CCC(=O)NC5=O)C4)CC3)nc21] | 44 |
| 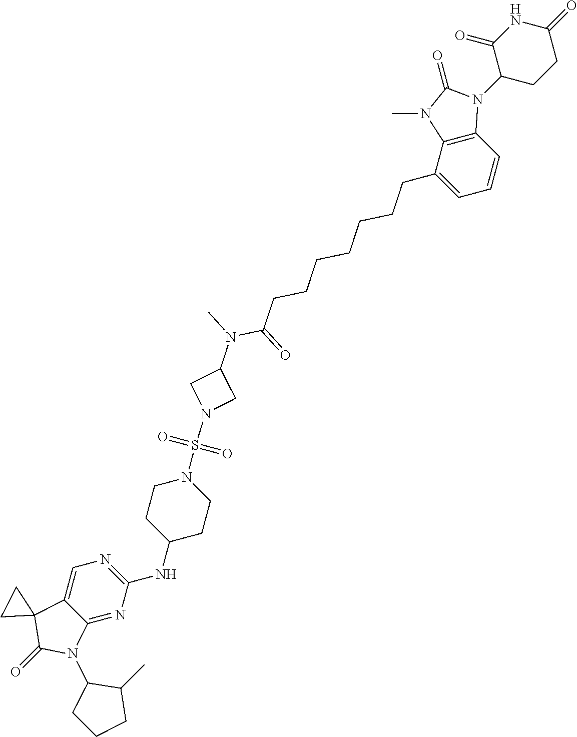 [SMILES: CC1CCCC1N1C(=O)C2(CC2)c2cnc(NC3CCN(S(=O)(=O)N4CC(N(C)C(=O)CCCCCCCc5cccc6c5n(C)c(=O)n6C5CCC(=O)NC5=O)C4)CC3)nc21] | 45 |
| 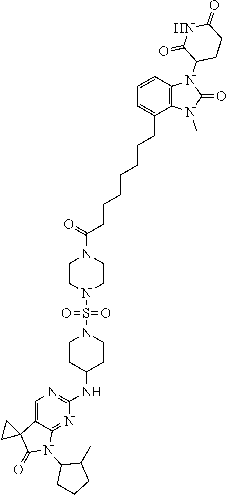 [SMILES: CC1CCCC1N1C(=O)C2(CC2)c2cnc(NC3CCN(S(=O)(=O)N4CCN(C(=O)CCCCCCCc5cccc6c5n(C)c(=O)n6C5CCC(=O)NC5=O)CC4)CC3)nc21] | 46 |
| 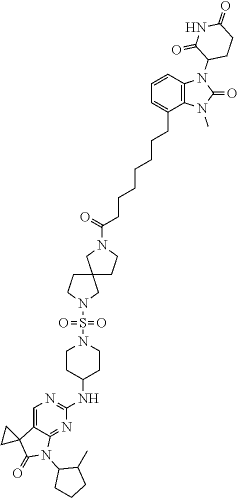 [SMILES: CC1CCCC1N1C(=O)C2(CC2)c2cnc(NC3CCN(S(=O)(=O)N4CCC5(CCN(C(=O)CCCCCCCc6cccc7c6n(C)c(=O)n7C6CCC(=O)NC6=O)C5)C4)CC3)nc21] | 47 |
| 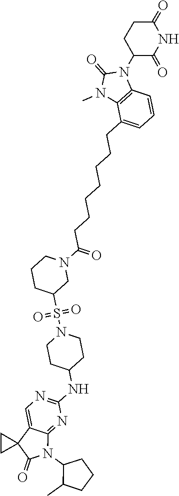 [SMILES: CC1CCCC1N1C(=O)C2(CC2)c2cnc(NC3CCN(S(=O)(=O)C4CCCN(C(=O)CCCCCCCc5cccc6c5n(C)c(=O)n6C5CCC(=O)NC5=O)C4)CC3)nc21] | 48 |
| 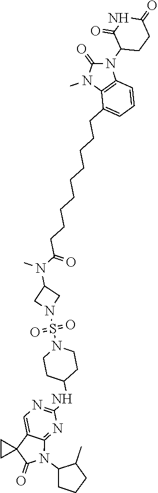 [SMILES: CC1CCCC1N1C(=O)C2(CC2)c2cnc(NC3CCN(S(=O)(=O)N4CC(N(C)C(=O)CCCCCCCCCc5cccc6c5n(C)c(=O)n6C5CCC(=O)NC5=O)C4)CC3)nc21] | 49 |
| 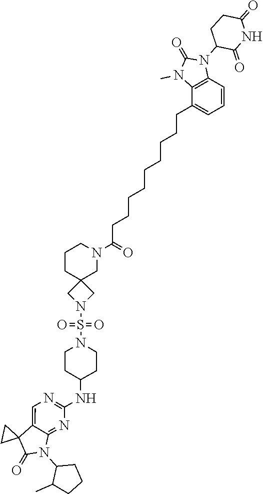 [SMILES: CC1CCCC1N1C(=O)C2(CC2)c2cnc(NC3CCN(S(=O)(=O)N4CC5(CCCN(C(=O)CCCCCCCCCc6cccc7c6n(C)c(=O)n7C6CCC(=O)NC6=O)C5)C4)CC3)nc21] | 50 |
| 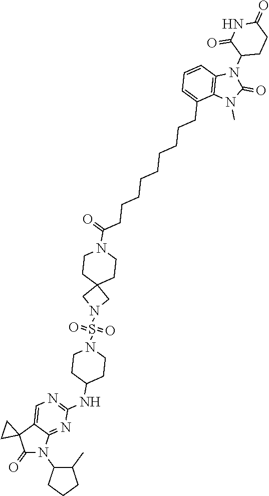 [SMILES: CC1CCCC1N1C(=O)C2(CC2)c2cnc(NC3CCN(S(=O)(=O)N4CC5(CCN(C(=O)CCCCCCCCCc6cccc7c6n(C)c(=O)n7C6CCC(=O)NC6=O)CC5)C4)CC3)nc21] | 51 |
| 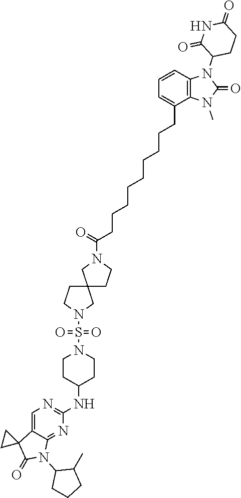 [SMILES: CC1CCCC1N1C(=O)C2(CC2)c2cnc(NC3CCN(S(=O)(=O)N4CCC5(CCN(C(=O)CCCCCCCCCc6cccc7c6n(C)c(=O)n7C6CCC(=O)NC6=O)C5)C4)CC3)nc21] | 52 |
| 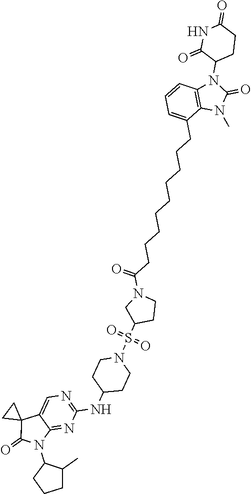 [SMILES: CC1CCCC1N1C(=O)C2(CC2)c2cnc(NC3CCN(S(=O)(=O)C4CCN(C(=O)CCCCCCCCCc5cccc6c5n(C)c(=O)n6C5CCC(=O)NC5=O)C4)CC3)nc21] | 53 |
| 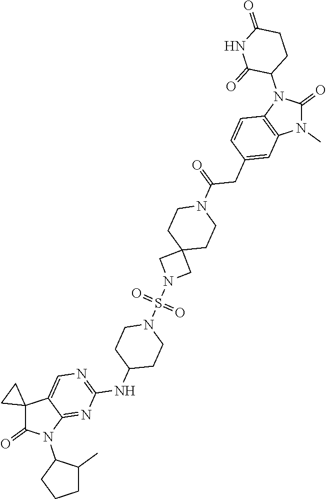 [SMILES: CC1CCCC1N1C(=O)C2(CC2)c2cnc(NC3CCN(S(=O)(=O)N4CC5(CCN(C(=O)Cc6ccc7c(c6)n(C)c(=O)n7C6CCC(=O)NC6=O)CC5)C4)CC3)nc21] | 54 |
| 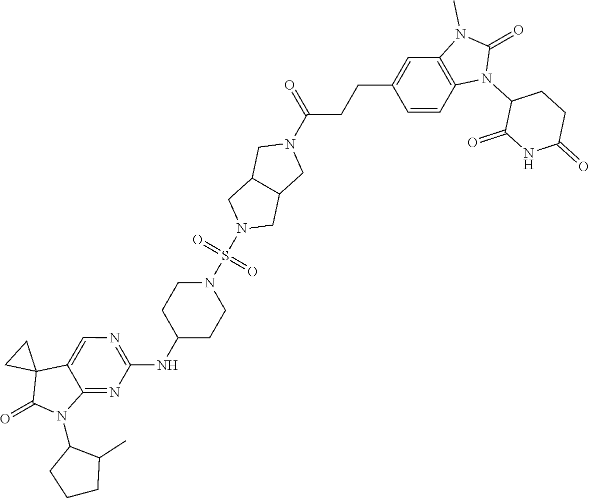 [SMILES: CC1CCCC1N1C(=O)C2(CC2)c2cnc(NC3CCN(S(=O)(=O)N4CC5CN(C(=O)CCc6ccc7c(c6)n(C)c(=O)n7C6CCC(=O)NC6=O)CC5C4)CC3)nc21] | 55 |
| 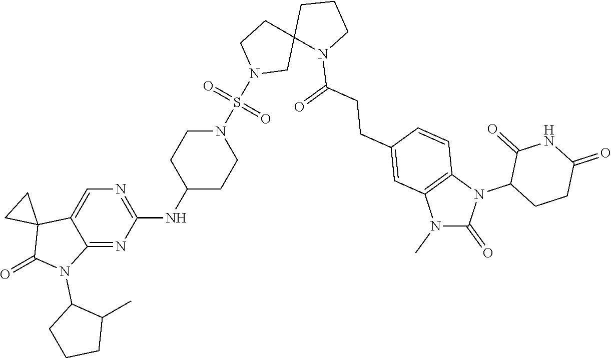 [SMILES: CC1CCCC1N1C(=O)C2(CC2)c2cnc(NC3CCN(S(=O)(=O)N4CCC5(CCCN5C(=O)CCc5ccc6c(c5)n(C)c(=O)n6C5CCC(=O)NC5=O)C4)CC3)nc21] | 56 |
| 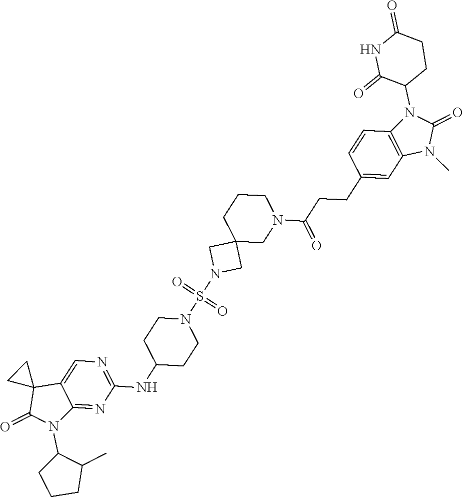 [SMILES: CC1CCCC1N1C(=O)C2(CC2)c2cnc(NC3CCN(S(=O)(=O)N4CC5(CCCN(C(=O)CCc6ccc7c(c6)n(C)c(=O)n7C6CCC(=O)NC6=O)C5)C4)CC3)nc21] | 57 |
| 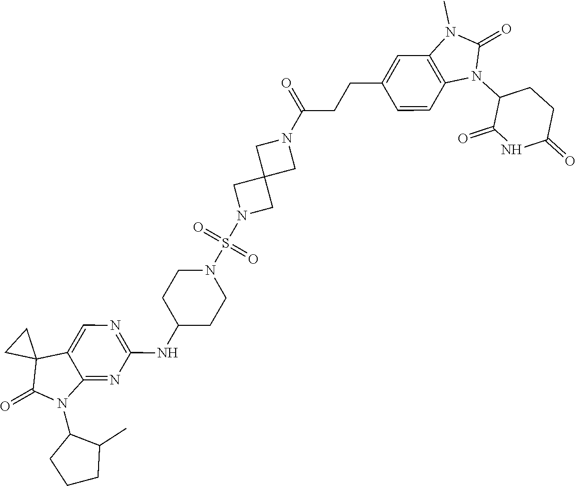 [SMILES: CC1CCCC1N1C(=O)C2(CC2)c2cnc(NC3CCN(S(=O)(=O)N4CC5(CN(C(=O)CCc6ccc7c(c6)n(C)c(=O)n7C6CCC(=O)NC6=O)C5)C4)CC3)nc21] | 58 |
| 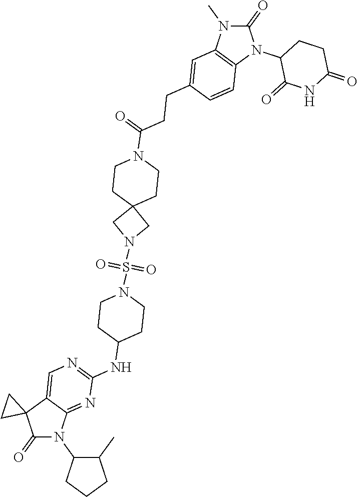 [SMILES: CC1CCCC1N1C(=O)C2(CC2)c2cnc(NC3CCN(S(=O)(=O)N4CC5(CCN(C(=O)CCc6ccc7c(c6)n(C)c(=O)n7C6CCC(=O)NC6=O)CC5)C4)CC3)nc21] | 59 |
| 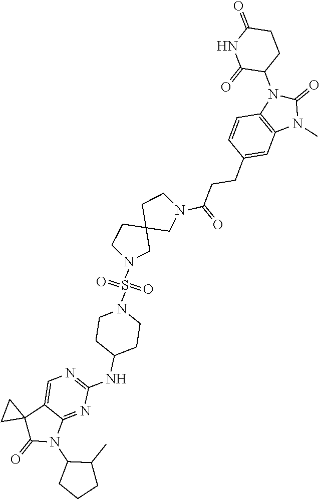 [SMILES: CC1CCCC1N1C(=O)C2(CC2)c2cnc(NC3CCN(S(=O)(=O)N4CCC5(CCN(C(=O)CCc6ccc7c(c6)n(C)c(=O)n7C6CCC(=O)NC6=O)C5)C4)CC3)nc21] | 60 |
| 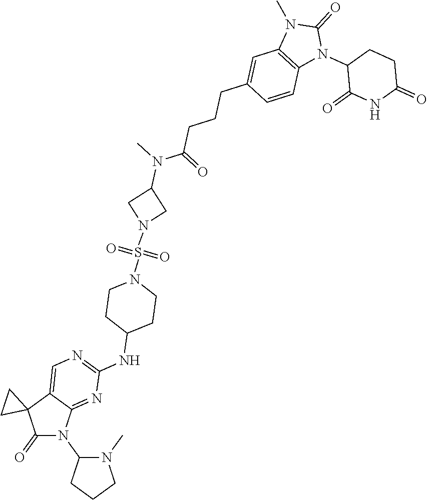 [SMILES: CN1CCCC1N1C(=O)C2(CC2)c2cnc(NC3CCN(S(=O)(=O)N4CC(N(C)C(=O)CCCc5ccc6c(c5)n(C)c(=O)n6C5CCC(=O)NC5=O)C4)CC3)nc21] | 61 |
| 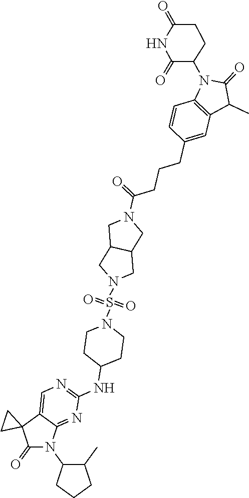 [SMILES: CC1C(=O)N(C2CCC(=O)NC2=O)c2ccc(CCCC(=O)N3CC4CN(S(=O)(=O)N5CCC(Nc6ncc7c(n6)N(C6CCCC6C)C(=O)C76CC6)CC5)CC4C3)cc21] | 62 |
| 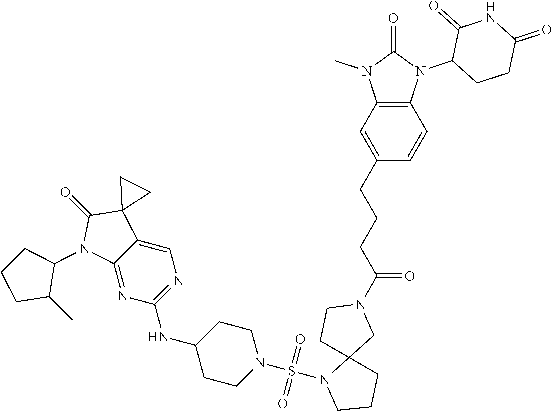 [SMILES: CC1CCCC1N1C(=O)C2(CC2)c2cnc(NC3CCN(S(=O)(=O)N4CCCC45CCN(C(=O)CCCc4ccc6c(c4)n(C)c(=O)n6C4CCC(=O)NC4=O)C5)CC3)nc21] | 63 |
| 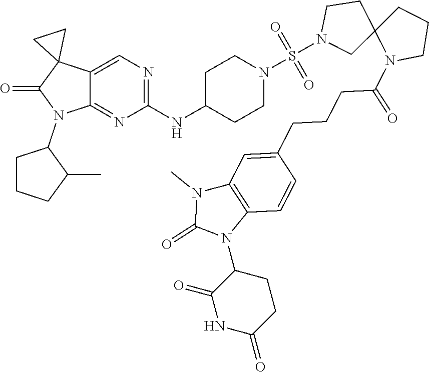 [SMILES: CC1CCCC1N1C(=O)C2(CC2)c2cnc(NC3CCN(S(=O)(=O)N4CCC5(CCCN5C(=O)CCCc5ccc6c(c5)n(C)c(=O)n6C5CCC(=O)NC5=O)C4)CC3)nc21] | 64 |
| 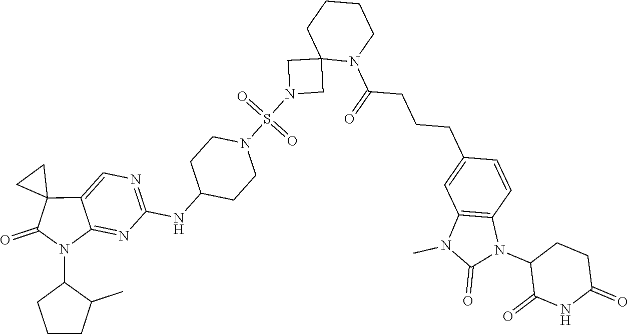 [SMILES: CC1CCCC1N1C(=O)C2(CC2)c2cnc(NC3CCN(S(=O)(=O)N4CC5(CCCCN5C(=O)CCCc5ccc6c(c5)n(C)c(=O)n6C5CCC(=O)NC5=O)C4)CC3)nc21] | 65 |
| 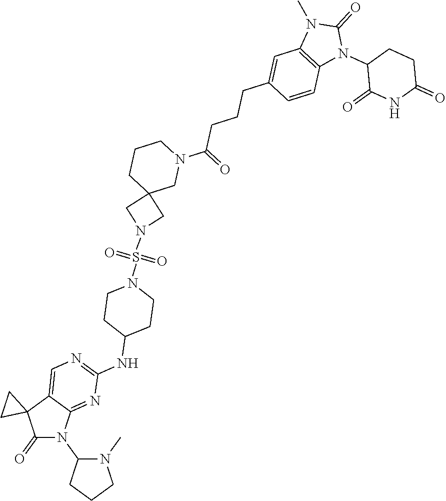 [SMILES: CN1CCCC1N1C(=O)C2(CC2)c2cnc(NC3CCN(S(=O)(=O)N4CC5(CCCN(C(=O)CCCc6ccc7c(c6)n(C)c(=O)n7C6CCC(=O)NC6=O)C5)C4)CC3)nc21] | 66 |
| 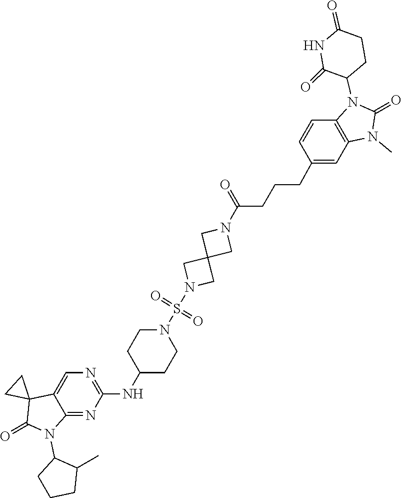 [SMILES: CC1CCCC1N1C(=O)C2(CC2)c2cnc(NC3CCN(S(=O)(=O)N4CC5(CN(C(=O)CCCc6ccc7c(c6)n(C)c(=O)n7C6CCC(=O)NC6=O)C5)C4)CC3)nc21] | 67 |
| 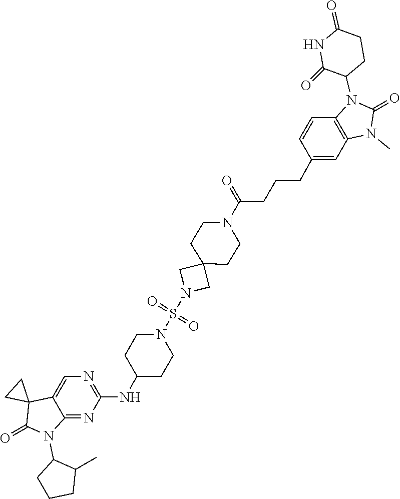 [SMILES: CC1CCCC1N1C(=O)C2(CC2)c2cnc(NC3CCN(S(=O)(=O)N4CC5(CCN(C(=O)CCCc6ccc7c(c6)n(C)c(=O)n7C6CCC(=O)NC6=O)CC5)C4)CC3)nc21] | 68 |
| 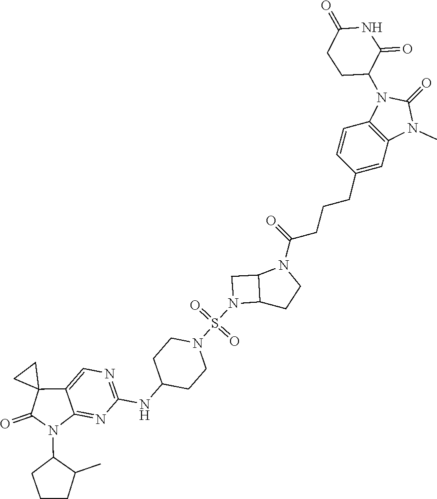 [SMILES: CC1CCCC1N1C(=O)C2(CC2)c2cnc(NC3CCN(S(=O)(=O)N4CC5C4CCN5C(=O)CCCc4ccc5c(c4)n(C)c(=O)n5C4CCC(=O)NC4=O)CC3)nc21] | 69 |
| 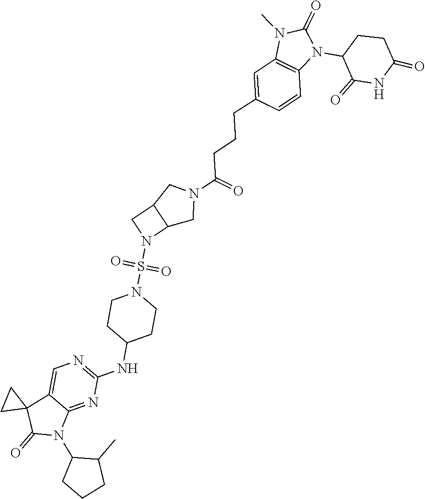 [SMILES: CC1CCCC1N1C(=O)C2(CC2)c2cnc(NC3CCN(S(=O)(=O)N4CC5CN(C(=O)CCCc6ccc7c(c6)n(C)c(=O)n7C6CCC(=O)NC6=O)CC54)CC3)nc21] | 70 |
| 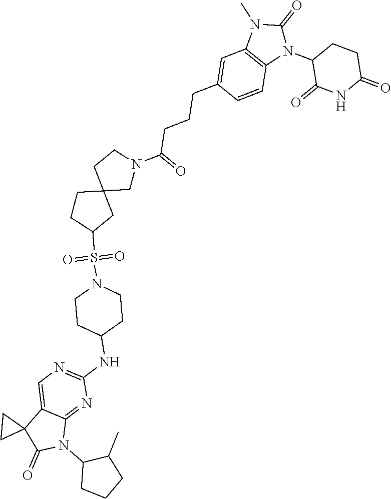 [SMILES: CC1CCCC1N1C(=O)C2(CC2)c2cnc(NC3CCN(S(=O)(=O)C4CCC5(CCN(C(=O)CCCc6ccc7c(c6)n(C)c(=O)n7C6CCC(=O)NC6=O)C5)C4)CC3)nc21] | 71 |
| 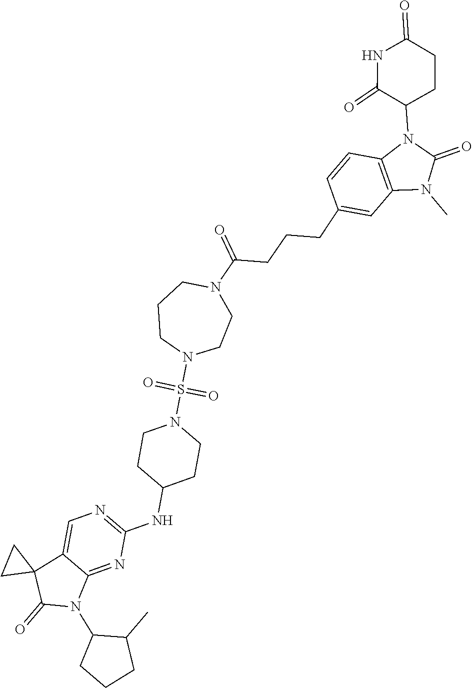 [SMILES: CC1CCCC1N1C(=O)C2(CC2)c2cnc(NC3CCN(S(=O)(=O)N4CCCN(C(=O)CCCc5ccc6c(c5)n(C)c(=O)n6C5CCC(=O)NC5=O)CC4)CC3)nc21] | 72 |
| 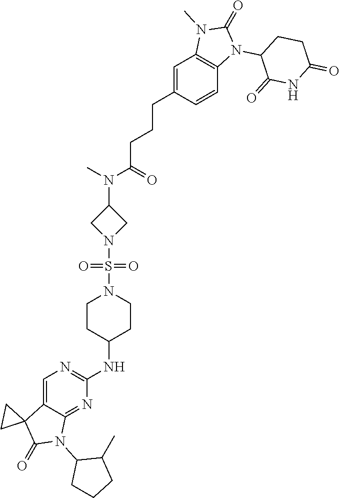 [SMILES: CC1CCCC1N1C(=O)C2(CC2)c2cnc(NC3CCN(S(=O)(=O)N4CC(N(C)C(=O)CCCc5ccc6c(c5)n(C)c(=O)n6C5CCC(=O)NC5=O)C4)CC3)nc21] | 73 |
| 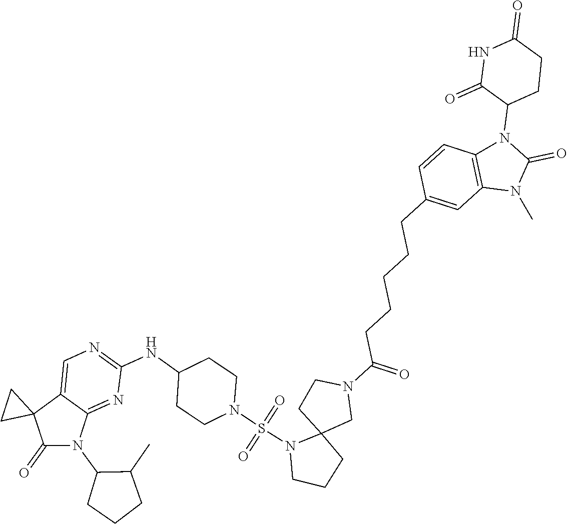 [SMILES: CC1CCCC1N1C(=O)C2(CC2)c2cnc(NC3CCN(S(=O)(=O)N4CCCC45CCN(C(=O)CCCCCc4ccc6c(c4)n(C)c(=O)n6C4CCC(=O)NC4=O)C5)CC3)nc21] | 74 |
| 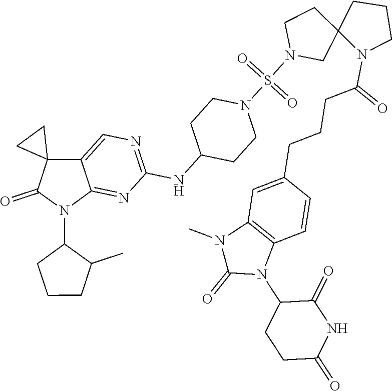 [SMILES: CC1CCCC1N1C(=O)C2(CC2)c2cnc(NC3CCN(S(=O)(=O)N4CCC5(CCCN5C(=O)CCCc5ccc6c(c5)n(C)c(=O)n6C5CCC(=O)NC5=O)C4)CC3)nc21] | 75 |
| 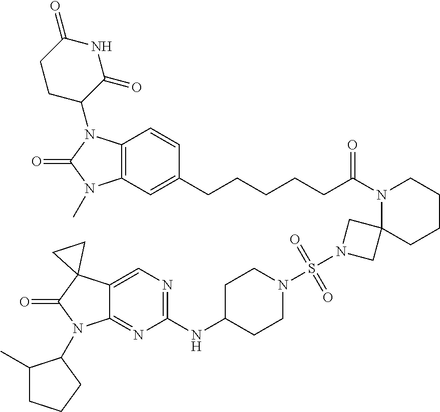 [SMILES: CC1CCCC1N1C(=O)C2(CC2)c2cnc(NC3CCN(S(=O)(=O)N4CC5(CCCCN5C(=O)CCCCCc5ccc6c(c5)n(C)c(=O)n6C5CCC(=O)NC5=O)C4)CC3)nc21] | 76 |
| 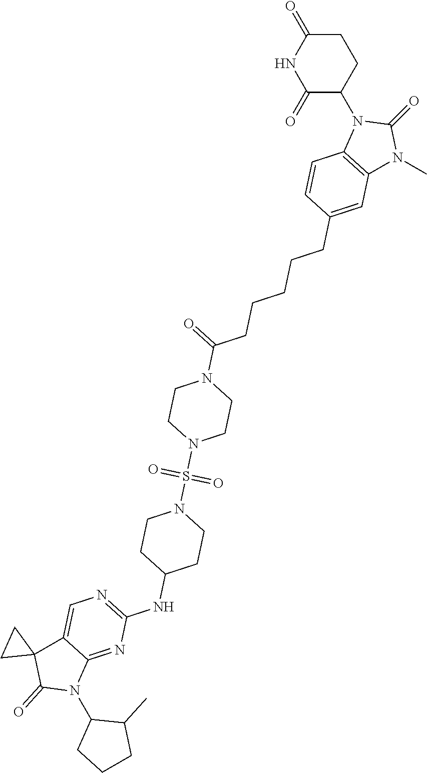 [SMILES: CC1CCCC1N1C(=O)C2(CC2)c2cnc(NC3CCN(S(=O)(=O)N4CCN(C(=O)CCCCCc5ccc6c(c5)n(C)c(=O)n6C5CCC(=O)NC5=O)CC4)CC3)nc21] | 77 |
| 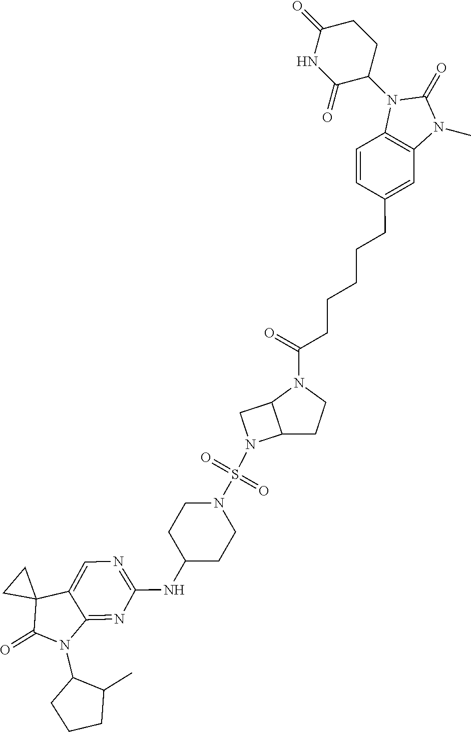 [SMILES: CC1CCCC1N1C(=O)C2(CC2)c2cnc(NC3CCN(S(=O)(=O)N4CC5C4CCN5C(=O)CCCCCc4ccc5c(c4)n(C)c(=O)n5C4CCC(=O)NC4=O)CC3)nc21] | 78 |
| 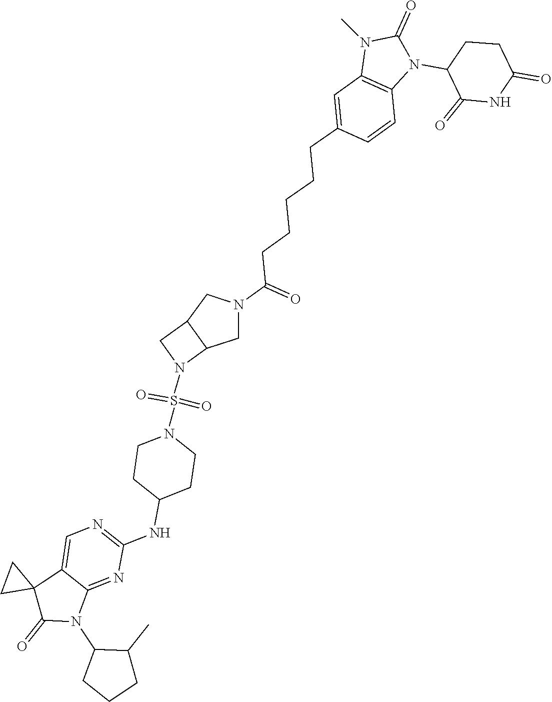 [SMILES: CC1CCCC1N1C(=O)C2(CC2)c2cnc(NC3CCN(S(=O)(=O)N4CC5CN(C(=O)CCCCCc6ccc7c(c6)n(C)c(=O)n7C6CCC(=O)NC6=O)CC54)CC3)nc21] | 79 |
| 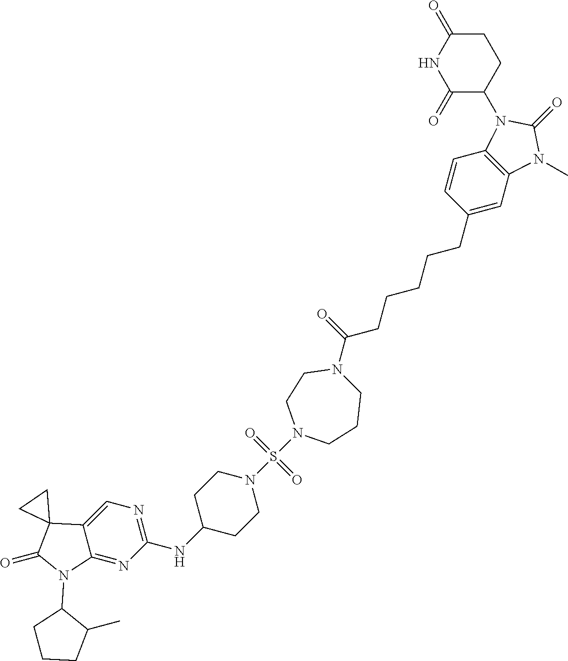 [SMILES: CC1CCCC1N1C(=O)C2(CC2)c2cnc(NC3CCN(S(=O)(=O)N4CCCN(C(=O)CCCCCc5ccc6c(c5)n(C)c(=O)n6C5CCC(=O)NC5=O)CC4)CC3)nc21] | 80 |
| 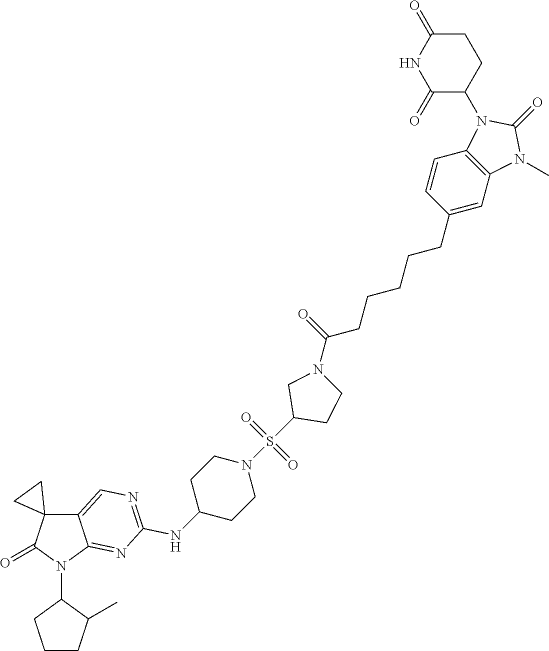 [SMILES: CC1CCCC1N1C(=O)C2(CC2)c2cnc(NC3CCN(S(=O)(=O)C4CCN(C(=O)CCCCCc5ccc6c(c5)n(C)c(=O)n6C5CCC(=O)NC5=O)C4)CC3)nc21] | 81 |
| 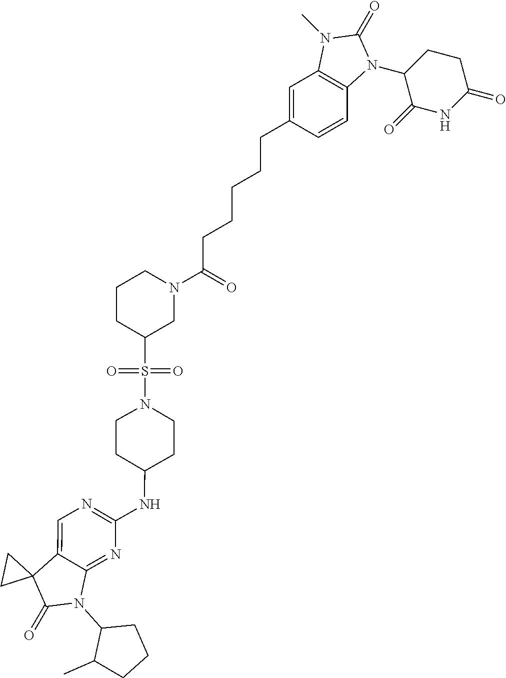 [SMILES: CC1CCCC1N1C(=O)C2(CC2)c2cnc(NC3CCN(S(=O)(=O)C4CCCN(C(=O)CCCCCc5ccc6c(c5)n(C)c(=O)n6C5CCC(=O)NC5=O)C4)CC3)nc21] | 82 |
| 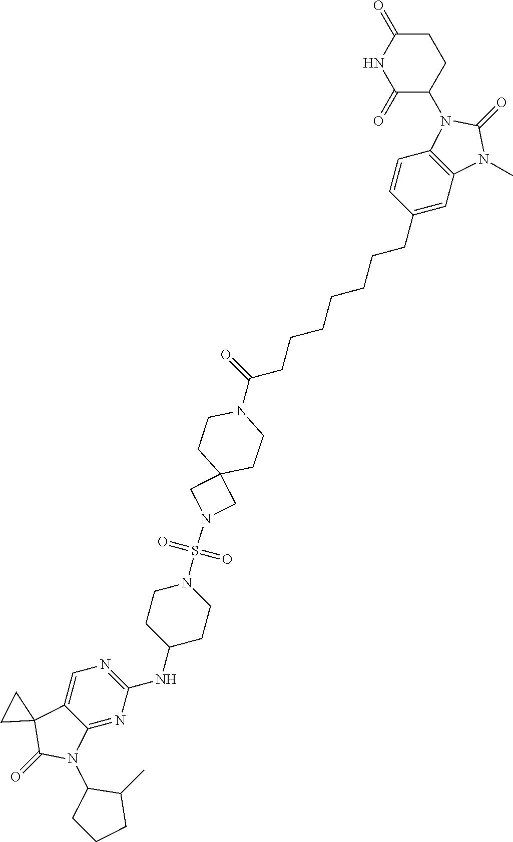 [SMILES: CC1CCCC1N1C(=O)C2(CC2)c2cnc(NC3CCN(S(=O)(=O)N4CC5(CCN(C(=O)CCCCCCCc6ccc7c(c6)n(C)c(=O)n7C6CCC(=O)NC6=O)CC5)C4)CC3)nc21] | 83 |
| 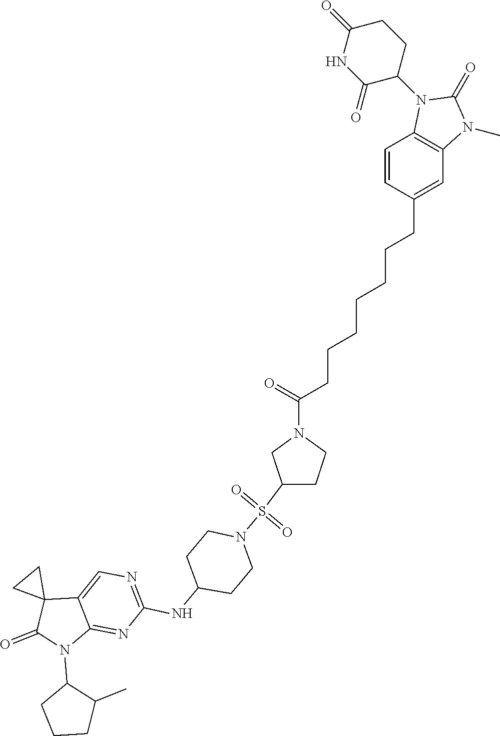 [SMILES: CC1CCCC1N1C(=O)C2(CC2)c2cnc(NC3CCN(S(=O)(=O)C4CCN(C(=O)CCCCCCCc5ccc6c(c5)n(C)c(=O)n6C5CCC(=O)NC5=O)C4)CC3)nc21] | 84 |
| 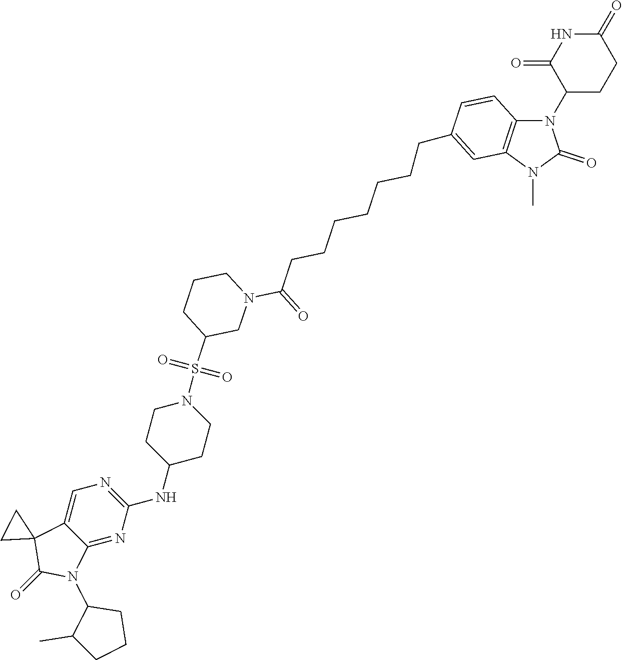 [SMILES: CC1CCCC1N1C(=O)C2(CC2)c2cnc(NC3CCN(S(=O)(=O)C4CCCN(C(=O)CCCCCCCc5ccc6c(c5)n(C)c(=O)n6C5CCC(=O)NC5=O)C4)CC3)nc21] | 85 |
| 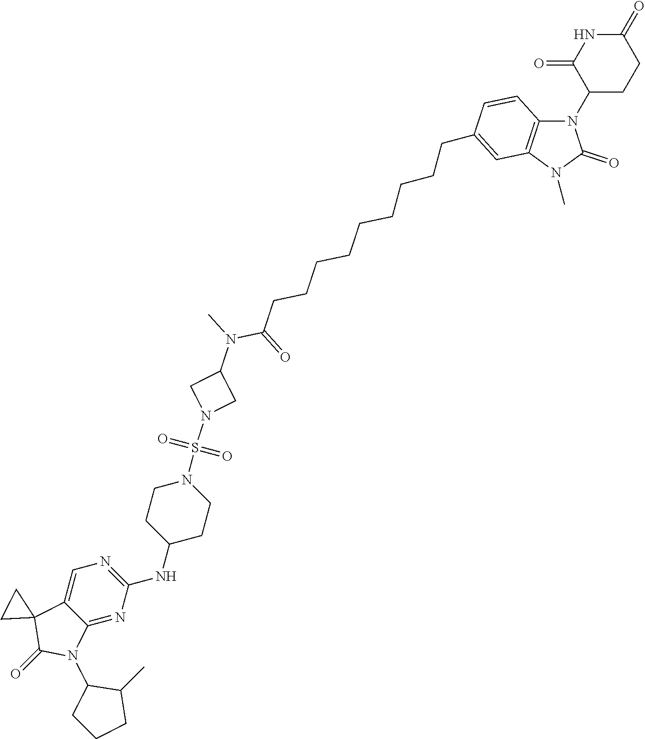 [SMILES: CC1CCCC1N1C(=O)C2(CC2)c2cnc(NC3CCN(S(=O)(=O)N4CC(N(C)C(=O)CCCCCCCCCc5ccc6c(c5)n(C)c(=O)n6C5CCC(=O)NC5=O)C4)CC3)nc21] | 86 |
| 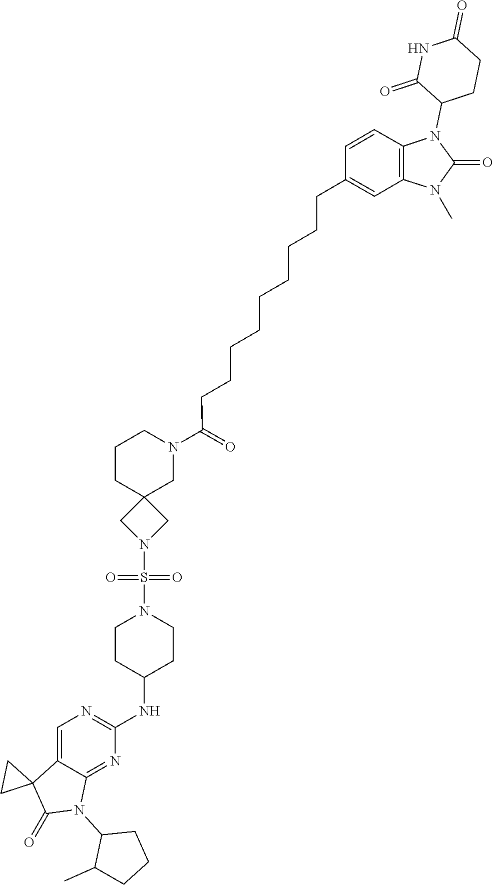 [SMILES: CC1CCCC1N1C(=O)C2(CC2)c2cnc(NC3CCN(S(=O)(=O)N4CC5(CCCN(C(=O)CCCCCCCCCc6ccc7c(c6)n(C)c(=O)n7C6CCC(=O)NC6=O)C5)C4)CC3)nc21] | 87 |
| 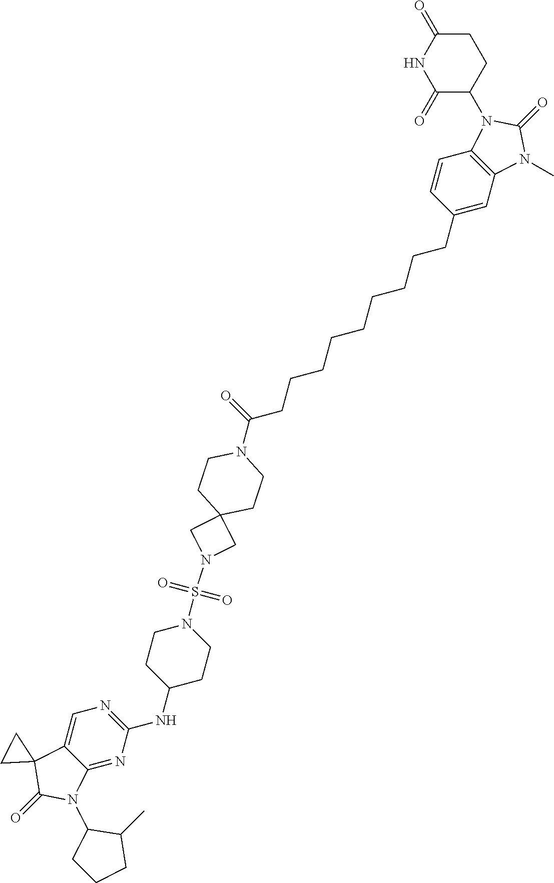 [SMILES: CC1CCCC1N1C(=O)C2(CC2)c2cnc(NC3CCN(S(=O)(=O)N4CC5(CCN(C(=O)CCCCCCCCCc6ccc7c(c6)n(C)c(=O)n7C6CCC(=O)NC6=O)CC5)C4)CC3)nc21] | 88 |
| 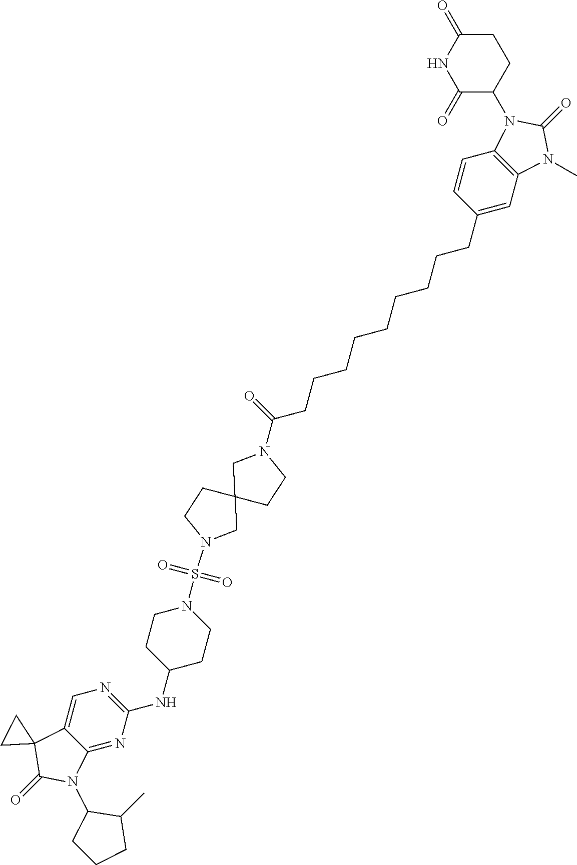 [SMILES: CC1CCCC1N1C(=O)C2(CC2)c2cnc(NC3CCN(S(=O)(=O)N4CCC5(CCN(C(=O)CCCCCCCCCc6ccc7c(c6)n(C)c(=O)n7C6CCC(=O)NC6=O)C5)C4)CC3)nc21] | 89 |
| 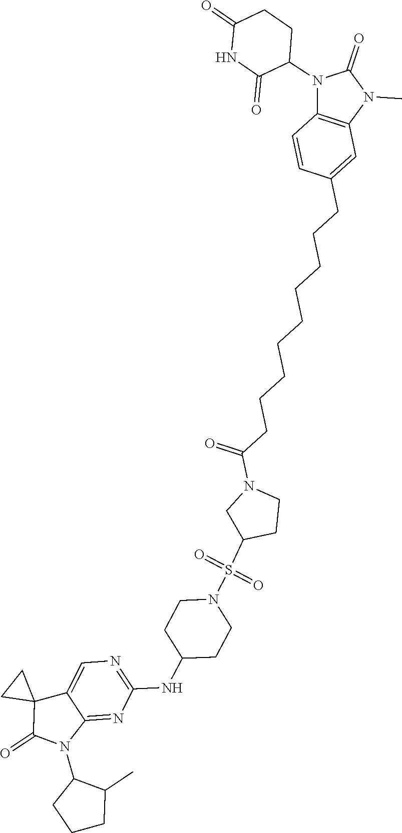 [SMILES: CC1CCCC1N1C(=O)C2(CC2)c2cnc(NC3CCN(S(=O)(=O)C4CCN(C(=O)CCCCCCCCCc5ccc6c(c5)n(C)c(=O)n6C5CCC(=O)NC5=O)C4)CC3)nc21] | 90 |
| 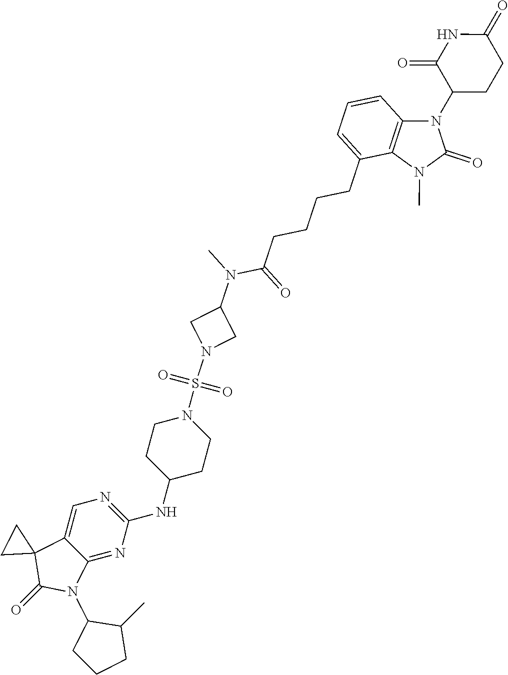 [SMILES: CC1CCCC1N1C(=O)C2(CC2)c2cnc(NC3CCN(S(=O)(=O)N4CC(N(C)C(=O)CCCCc5cccc6c5n(C)c(=O)n6C5CCC(=O)NC5=O)C4)CC3)nc21] | 91 |
| 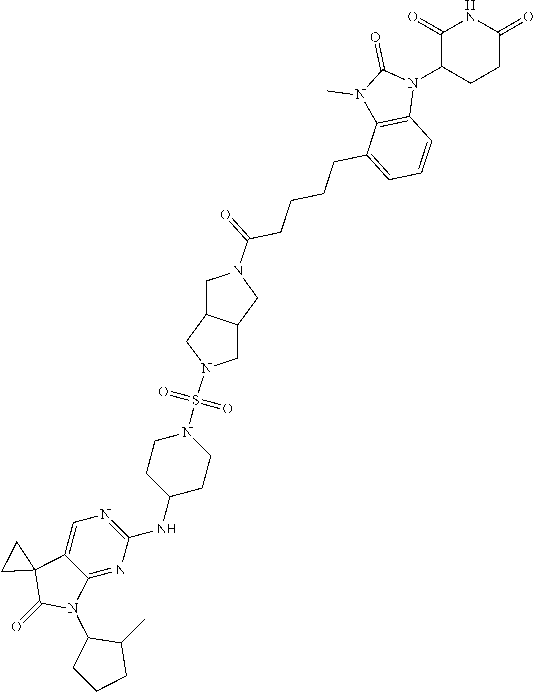 [SMILES: CC1CCCC1N1C(=O)C2(CC2)c2cnc(NC3CCN(S(=O)(=O)N4CC5CN(C(=O)CCCCc6cccc7c6n(C)c(=O)n7C6CCC(=O)NC6=O)CC5C4)CC3)nc21] | 92 |
| 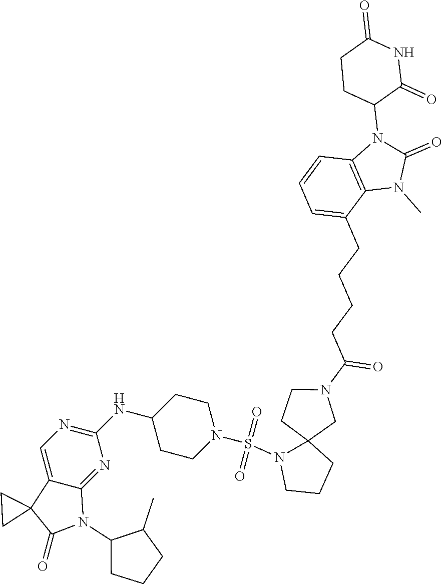 [SMILES: CC1CCCC1N1C(=O)C2(CC2)c2cnc(NC3CCN(S(=O)(=O)N4CCCC45CCN(C(=O)CCCCc4cccc6c4n(C)c(=O)n6C4CCC(=O)NC4=O)C5)CC3)nc21] | 93 |
| 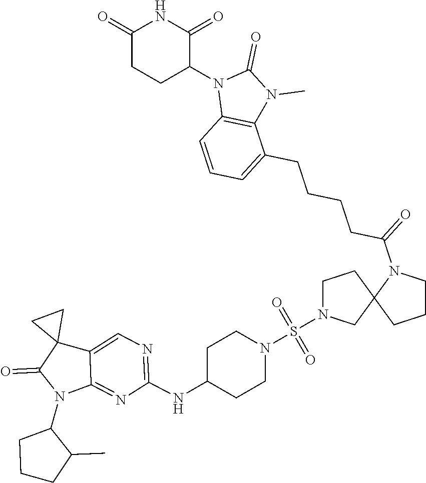 [SMILES: CC1CCCC1N1C(=O)C2(CC2)c2cnc(NC3CCN(S(=O)(=O)N4CCC5(CCCN5C(=O)CCCCc5cccc6c5n(C)c(=O)n6C5CCC(=O)NC5=O)C4)CC3)nc21] | 94 |
| 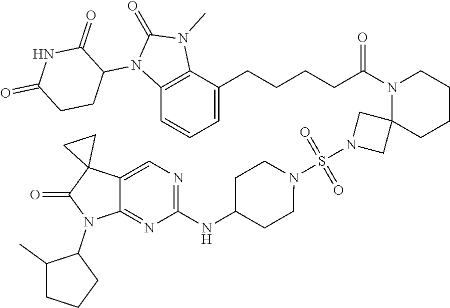 [SMILES: CC1CCCC1N1C(=O)C2(CC2)c2cnc(NC3CCN(S(=O)(=O)N4CC5(CCCCN5C(=O)CCCCc5cccc6c5n(C)c(=O)n6C5CCC(=O)NC5=O)C4)CC3)nc21] | 95 |
| 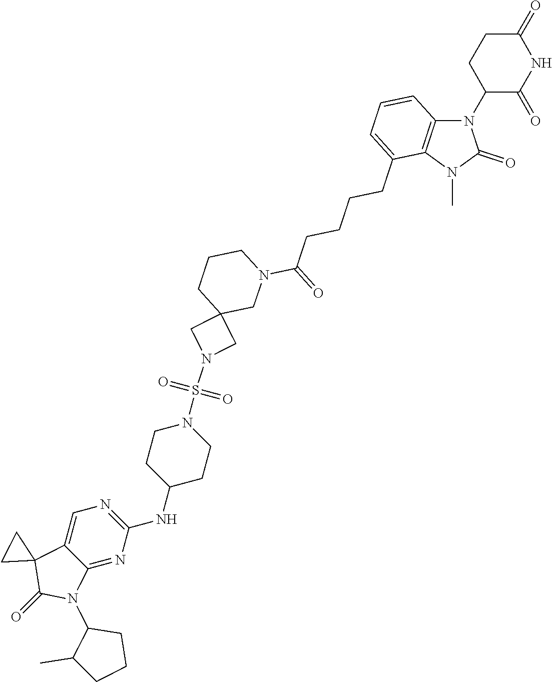 [SMILES: CC1CCCC1N1C(=O)C2(CC2)c2cnc(NC3CCN(S(=O)(=O)N4CC5(CCCN(C(=O)CCCCc6cccc7c6n(C)c(=O)n7C6CCC(=O)NC6=O)C5)C4)CC3)nc21] | 96 |
| 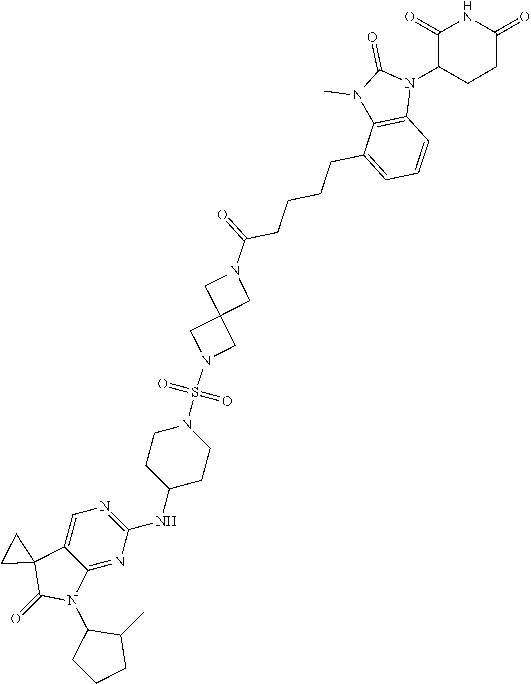 [SMILES: CC1CCCC1N1C(=O)C2(CC2)c2cnc(NC3CCN(S(=O)(=O)N4CC5(CN(C(=O)CCCCc6cccc7c6n(C)c(=O)n7C6CCC(=O)NC6=O)C5)C4)CC3)nc21] | 97 |
| 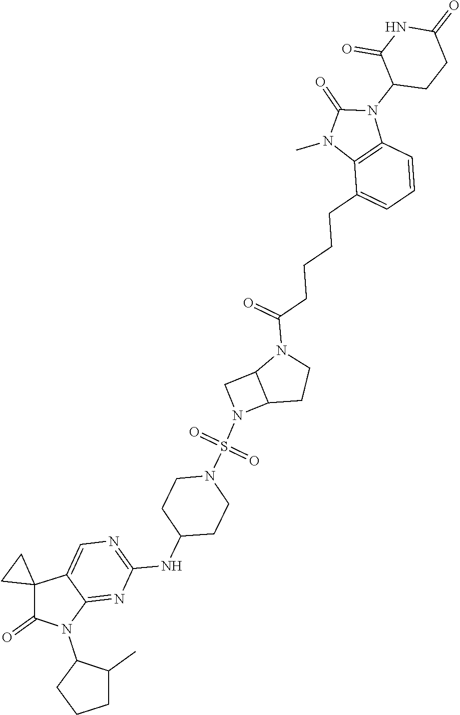 [SMILES: CC1CCCC1N1C(=O)C2(CC2)c2cnc(NC3CCN(S(=O)(=O)N4CC5C4CCN5C(=O)CCCCc4cccc5c4n(C)c(=O)n5C4CCC(=O)NC4=O)CC3)nc21] | 98 |
| 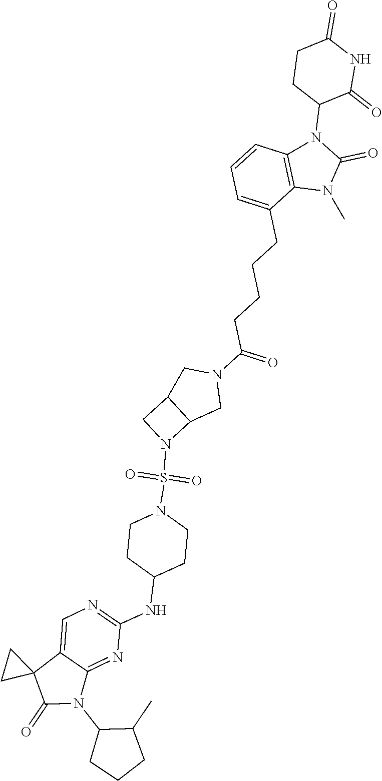 [SMILES: CC1CCCC1N1C(=O)C2(CC2)c2cnc(NC3CCN(S(=O)(=O)N4CC5CN(C(=O)CCCCc6cccc7c6n(C)c(=O)n7C6CCC(=O)NC6=O)CC54)CC3)nc21] | 99 |
| 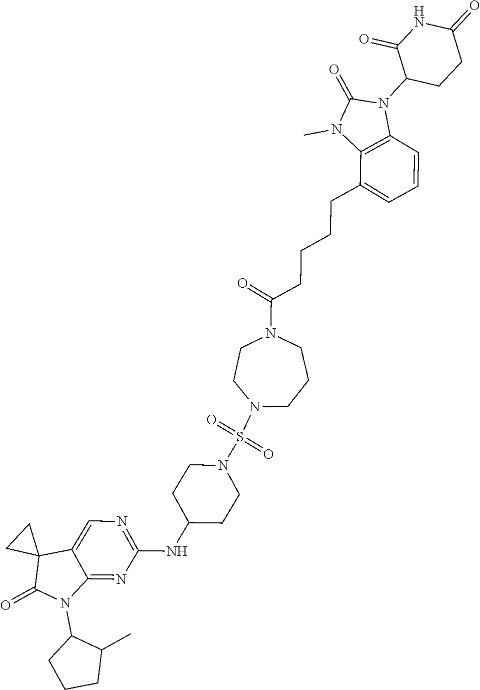 [SMILES: CC1CCCC1N1C(=O)C2(CC2)c2cnc(NC3CCN(S(=O)(=O)N4CCCN(C(=O)CCCCc5cccc6c5n(C)c(=O)n6C5CCC(=O)NC5=O)CC4)CC3)nc21] | 100 |
| 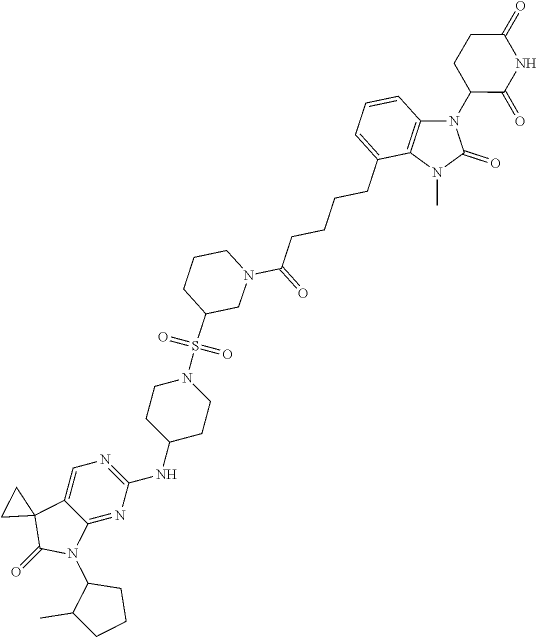 [SMILES: CC1CCCC1N1C(=O)C2(CC2)c2cnc(NC3CCN(S(=O)(=O)C4CCCN(C(=O)CCCCc5cccc6c5n(C)c(=O)n6C5CCC(=O)NC5=O)C4)CC3)nc21] | 101 |
| 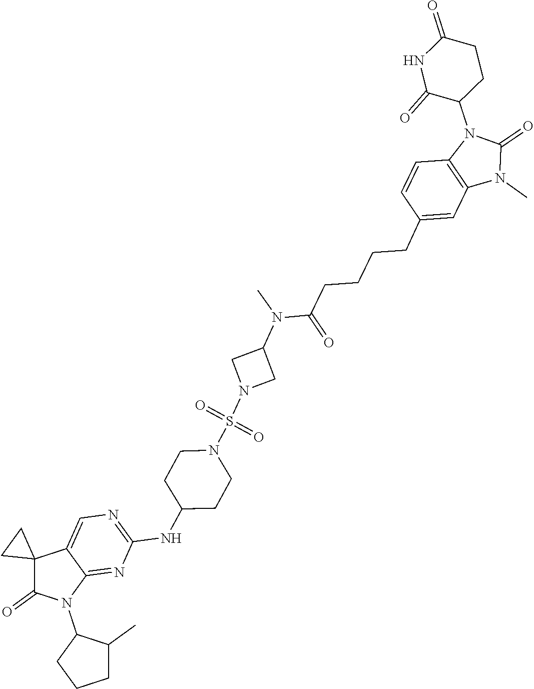 [SMILES: CC1CCCC1N1C(=O)C2(CC2)c2cnc(NC3CCN(S(=O)(=O)N4CC(N(C)C(=O)CCCCc5ccc6c(c5)n(C)c(=O)n6C5CCC(=O)NC5=O)C4)CC3)nc21] | 102 |
| 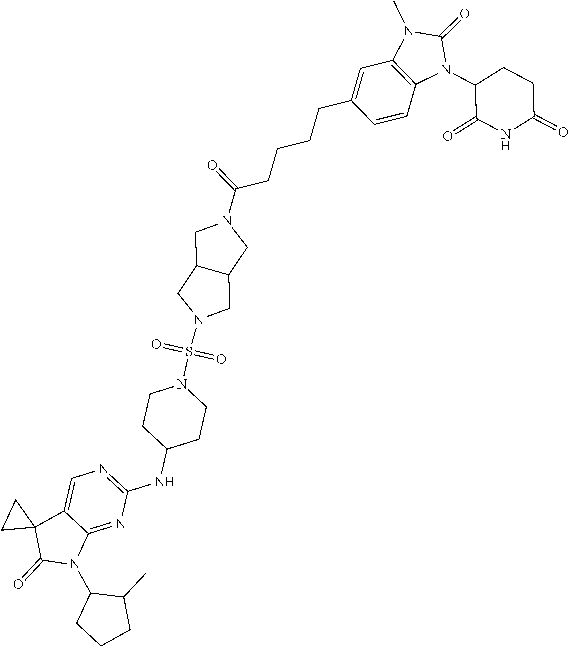 [SMILES: CC1CCCC1N1C(=O)C2(CC2)c2cnc(NC3CCN(S(=O)(=O)N4CC5CN(C(=O)CCCCc6ccc7c(c6)n(C)c(=O)n7C6CCC(=O)NC6=O)CC5C4)CC3)nc21] | 103 |
| 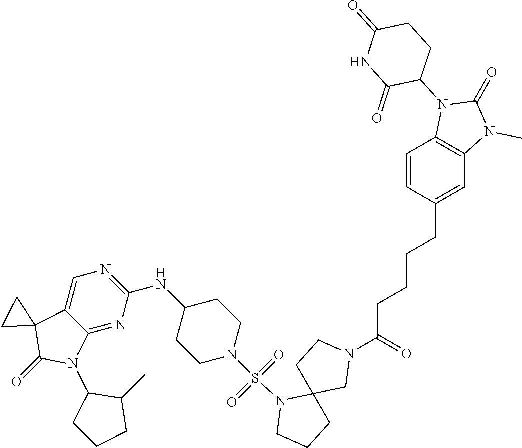 [SMILES: CC1CCCC1N1C(=O)C2(CC2)c2cnc(NC3CCN(S(=O)(=O)N4CCCC45CCN(C(=O)CCCCc4ccc6c(c4)n(C)c(=O)n6C4CCC(=O)NC4=O)C5)CC3)nc21] | 104 |
| 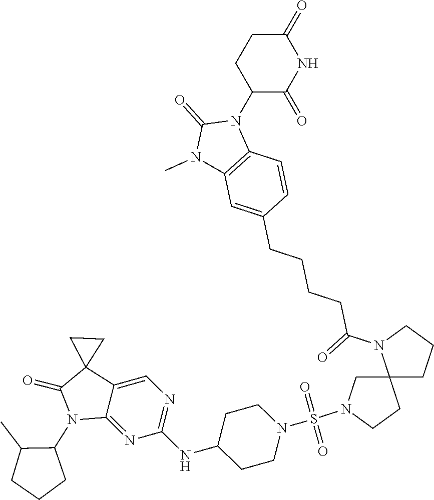 [SMILES: CC1CCCC1N1C(=O)C2(CC2)c2cnc(NC3CCN(S(=O)(=O)N4CCC5(CCCN5C(=O)CCCCc5ccc6c(c5)n(C)c(=O)n6C5CCC(=O)NC5=O)C4)CC3)nc21] | 105 |
| 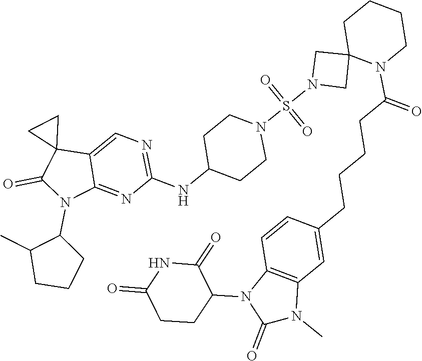 [SMILES: CC1CCCC1N1C(=O)C2(CC2)c2cnc(NC3CCN(S(=O)(=O)N4CC5(CCCCN5C(=O)CCCCc5ccc6c(c5)n(C)c(=O)n6C5CCC(=O)NC5=O)C4)CC3)nc21] | 106 |
| 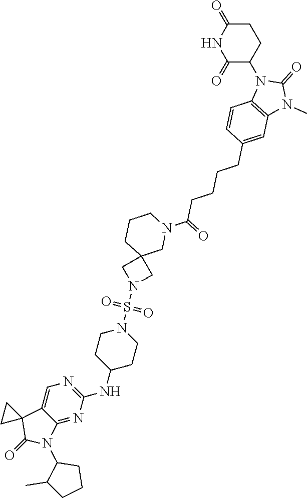 [SMILES: CC1CCCC1N1C(=O)C2(CC2)c2cnc(NC3CCN(S(=O)(=O)N4CC5(CCCN(C(=O)CCCCc6ccc7c(c6)n(C)c(=O)n7C6CCC(=O)NC6=O)C5)C4)CC3)nc21] | 107 |
| 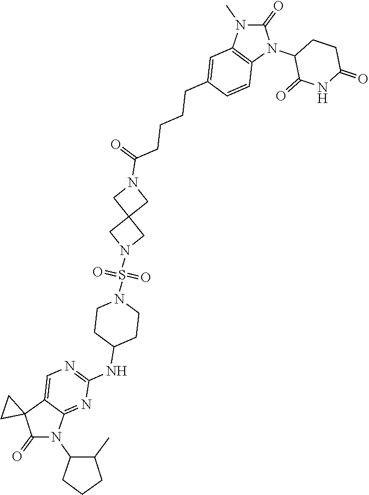 [SMILES: CC1CCCC1N1C(=O)C2(CC2)c2cnc(NC3CCN(S(=O)(=O)N4CC5(CN(C(=O)CCCCc6ccc7c(c6)n(C)c(=O)n7C6CCC(=O)NC6=O)C5)C4)CC3)nc21] | 108 |
| 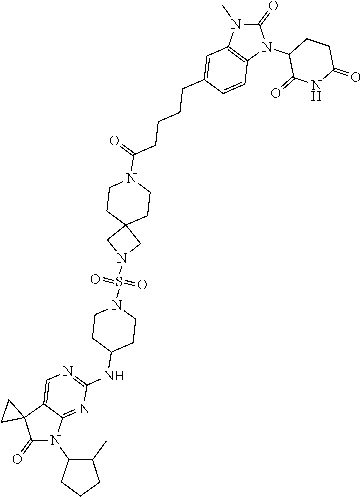 [SMILES: CC1CCCC1N1C(=O)C2(CC2)c2cnc(NC3CCN(S(=O)(=O)N4CC5(CCN(C(=O)CCCCc6ccc7c(c6)n(C)c(=O)n7C6CCC(=O)NC6=O)CC5)C4)CC3)nc21] | 109 |
| 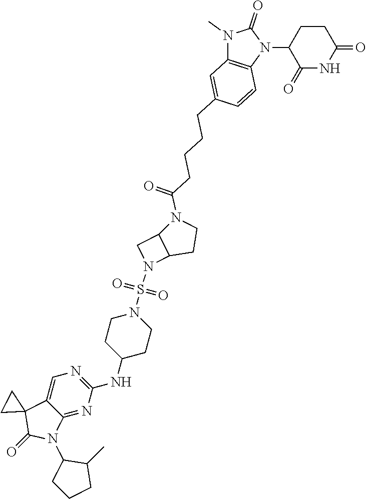 [SMILES: CC1CCCC1N1C(=O)C2(CC2)c2cnc(NC3CCN(S(=O)(=O)N4CC5C4CCN5C(=O)CCCCc4ccc5c(c4)n(C)c(=O)n5C4CCC(=O)NC4=O)CC3)nc21] | 110 |
| 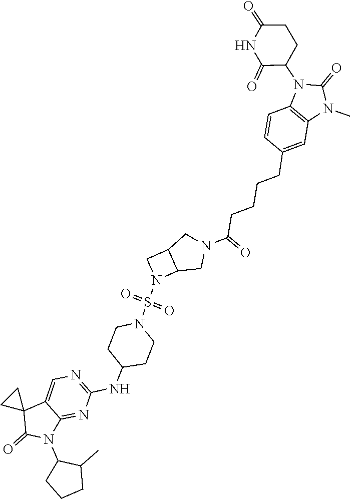 [SMILES: CC1CCCC1N1C(=O)C2(CC2)c2cnc(NC3CCN(S(=O)(=O)N4CC5CN(C(=O)CCCCc6ccc7c(c6)n(C)c(=O)n7C6CCC(=O)NC6=O)CC54)CC3)nc21] | 111 |
| 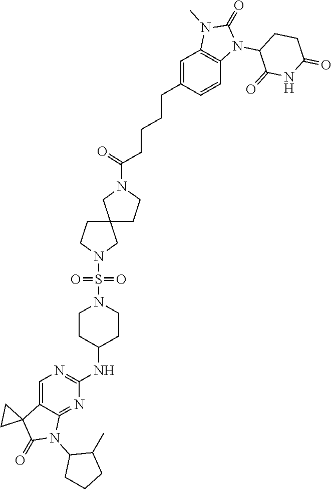 [SMILES: CC1CCCC1N1C(=O)C2(CC2)c2cnc(NC3CCN(S(=O)(=O)N4CCC5(CCN(C(=O)CCCCc6ccc7c(c6)n(C)c(=O)n7C6CCC(=O)NC6=O)C5)C4)CC3)nc21] | 112 |
| 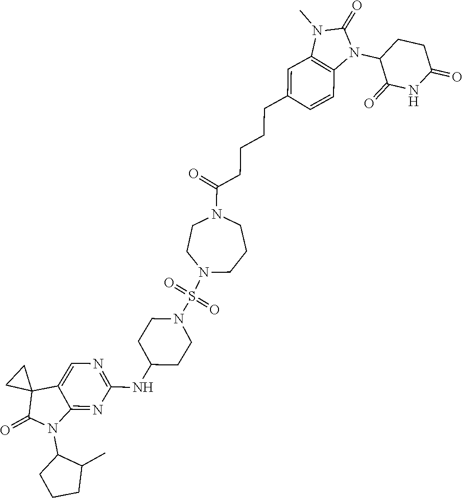 [SMILES: CC1CCCC1N1C(=O)C2(CC2)c2cnc(NC3CCN(S(=O)(=O)N4CCCN(C(=O)CCCCc5ccc6c(c5)n(C)c(=O)n6C5CCC(=O)NC5=O)CC4)CC3)nc21] | 113 |
| 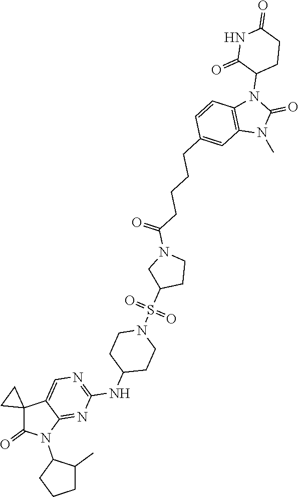 [SMILES: CC1CCCC1N1C(=O)C2(CC2)c2cnc(NC3CCN(S(=O)(=O)C4CCN(C(=O)CCCCc5ccc6c(c5)n(C)c(=O)n6C5CCC(=O)NC5=O)C4)CC3)nc21] | 114 |
| 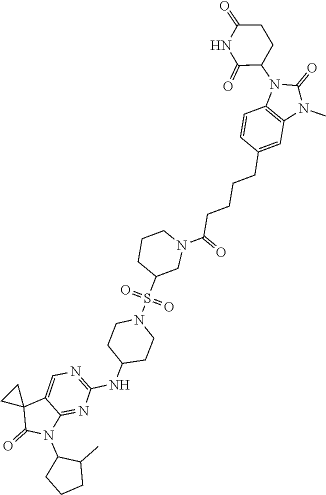 [SMILES: CC1CCCC1N1C(=O)C2(CC2)c2cnc(NC3CCN(S(=O)(=O)C4CCCN(C(=O)CCCCc5ccc6c(c5)n(C)c(=O)n6C5CCC(=O)NC5=O)C4)CC3)nc21] | 115 |
| 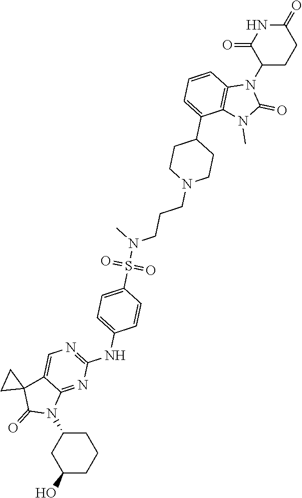 [SMILES: CN(CCCN1CCC(c2cccc3c2n(C)c(=O)n3C2CCC(=O)NC2=O)CC1)S(=O)(=O)c1ccc(Nc2ncc3c(n2)N([C@@H]2CCC[C@@H](O)C2)C(=O)C32CC2)cc1] | 116 |
| 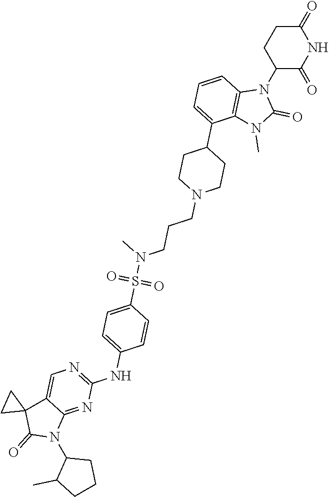 [SMILES: CC1CCCC1N1C(=O)C2(CC2)c2cnc(Nc3ccc(S(=O)(=O)N(C)CCCN4CCC(c5cccc6c5n(C)c(=O)n6C5CCC(=O)NC5=O)CC4)cc3)nc21] | 117 |
| 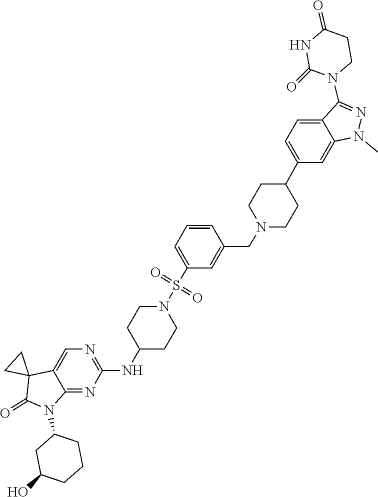 [SMILES: Cn1nc(N2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)c4)CC3)cc21] | 118 |
| 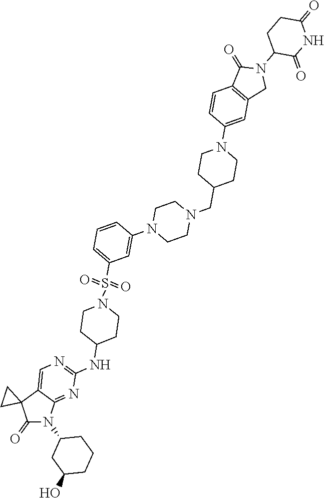 [SMILES: O=C1CCC(N2Cc3cc(N4CCC(CN5CCN(c6cccc(S(=O)(=O)N7CCC(Nc8ncc9c(n8)N([C@@H]8CCC[C@@H](O)C8)C(=O)C98CC8)CC7)c6)CC5)CC4)ccc3C2=O)C(=O)N1] | 119 |
| 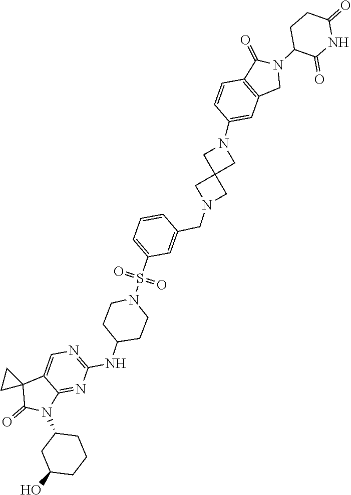 [SMILES: O=C1CCC(N2Cc3cc(N4CC5(CN(Cc6cccc(S(=O)(=O)N7CCC(Nc8ncc9c(n8)N([C@@H]8CCC[C@@H](O)C8)C(=O)C98CC8)CC7)c6)C5)C4)ccc3C2=O)C(=O)N1] | 120 |
| 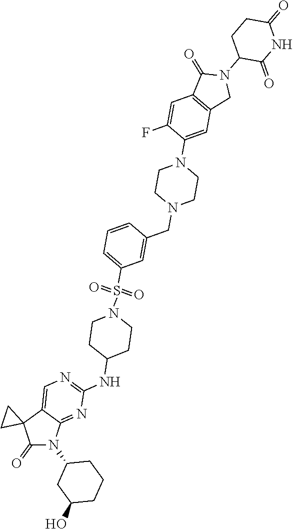 [SMILES: O=C1CCC(N2Cc3cc(N4CCN(Cc5cccc(S(=O)(=O)N6CCC(Nc7ncc8c(n7)N([C@@H]7CCC[C@@H](O)C7)C(=O)C87CC7)CC6)c5)CC4)c(F)cc3C2=O)C(=O)N1] | 121 |
| 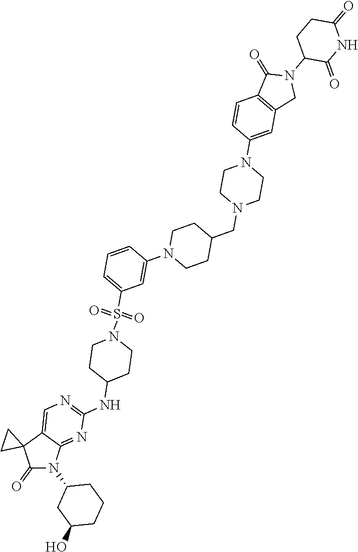 [SMILES: O=C1CCC(N2Cc3cc(N4CCN(CC5CCN(c6cccc(S(=O)(=O)N7CCC(Nc8ncc9c(n8)N([C@@H]8CCC[C@@H](O)C8)C(=O)C98CC8)CC7)c6)CC5)CC4)ccc3C2=O)C(=O)N1] | 122 |
| 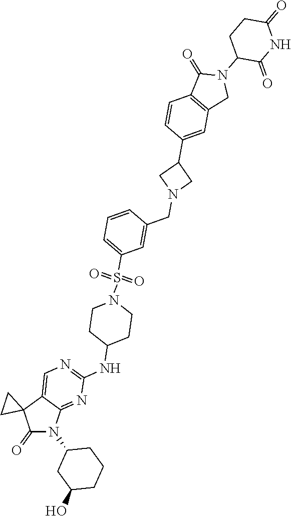 [SMILES: O=C1CCC(N2Cc3cc(C4CN(Cc5cccc(S(=O)(=O)N6CCC(Nc7ncc8c(n7)N([C@@H]7CCC[C@@H](O)C7)C(=O)C87CC7)CC6)c5)C4)ccc3C2=O)C(=O)N1] | 123 |
| 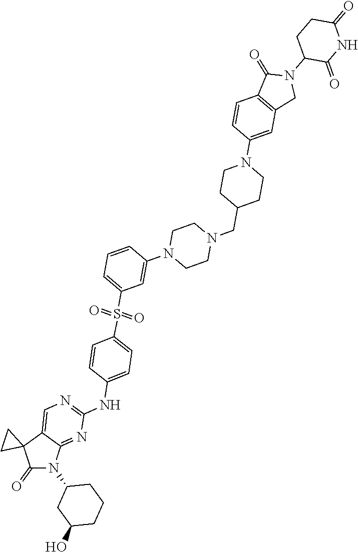 [SMILES: O=C1CCC(N2Cc3cc(N4CCC(CN5CCN(c6cccc(S(=O)(=O)c7ccc(Nc8ncc9c(n8)N([C@@H]8CCC[C@@H](O)C8)C(=O)C98CC8)cc7)c6)CC5)CC4)ccc3C2=O)C(=O)N1] | 124 |
| 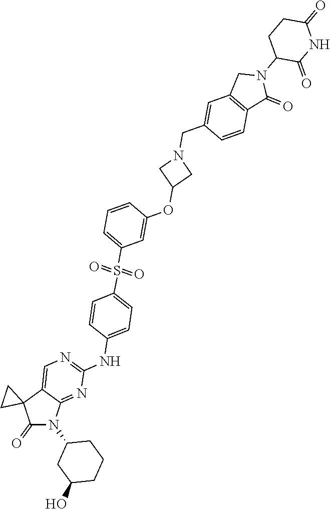 [SMILES: O=C1CCC(N2Cc3cc(CN4CC(Oc5cccc(S(=O)(=O)c6ccc(Nc7ncc8c(n7)N([C@@H]7CCC[C@@H](O)C7)C(=O)C87CC7)cc6)c5)C4)ccc3C2=O)C(=O)N1] | 125 |
| 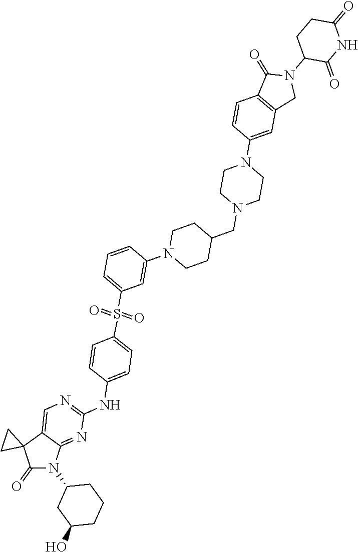 [SMILES: O=C1CCC(N2Cc3cc(N4CCN(CC5CCN(c6cccc(S(=O)(=O)c7ccc(Nc8ncc9c(n8)N([C@@H]8CCC[C@@H](O)C8)C(=O)C98CC8)cc7)c6)CC5)CC4)ccc3C2=O)C(=O)N1] | 126 |
| 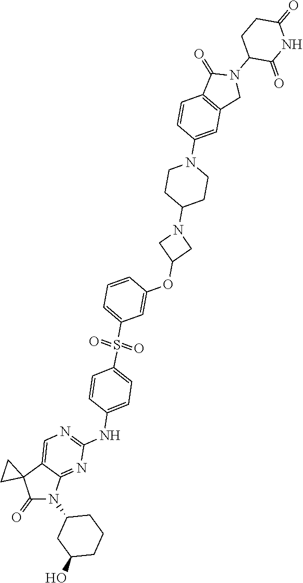 [SMILES: O=C1CCC(N2Cc3cc(N4CCC(N5CC(Oc6cccc(S(=O)(=O)c7ccc(Nc8ncc9c(n8)N([C@@H]8CCC[C@@H](O)C8)C(=O)C98CC8)cc7)c6)C5)CC4)ccc3C2=O)C(=O)N1] | 127 |
| 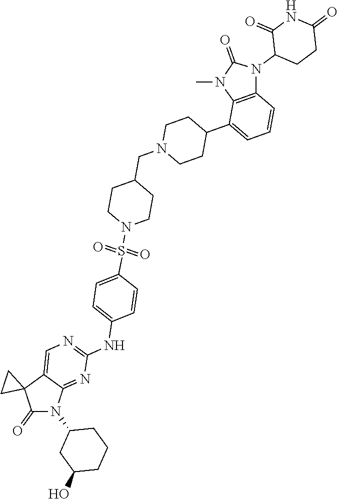 [SMILES: Cn1c(=O)n(C2CCC(=O)NC2=O)c2cccc(C3CCN(CC4CCN(S(=O)(=O)c5ccc(Nc6ncc7c(n6)N([C@@H]6CCC[C@@H](O)C6)C(=O)C76CC6)cc5)CC4)CC3)c21] | 128 |
| 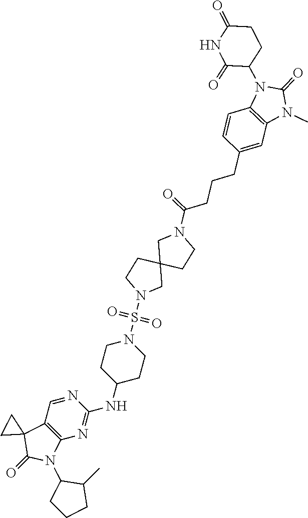 [SMILES: CC1CCCC1N1C(=O)C2(CC2)c2cnc(NC3CCN(S(=O)(=O)N4CCC5(CCN(C(=O)CCCc6ccc7c(c6)n(C)c(=O)n7C6CCC(=O)NC6=O)C5)C4)CC3)nc21] | 129 |
| 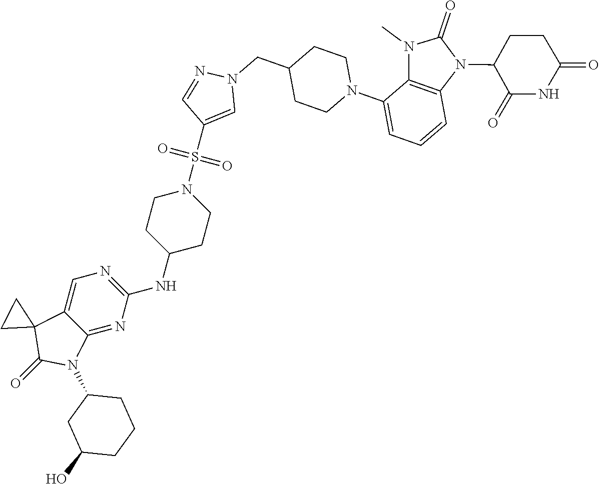 [SMILES: Cn1c(=O)n(C2CCC(=O)NC2=O)c2cccc(N3CCC(Cn4cc(S(=O)(=O)N5CCC(Nc6ncc7c(n6)N([C@@H]6CCC[C@@H](O)C6)C(=O)C76CC6)CC5)cn4)CC3)c21] | 130 |
| 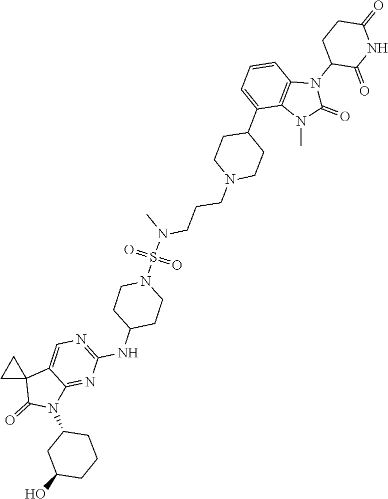 [SMILES: CN(CCCN1CCC(c2cccc3c2n(C)c(=O)n3C2CCC(=O)NC2=O)CC1)S(=O)(=O)N1CCC(Nc2ncc3c(n2)N([C@@H]2CCC[C@@H](O)C2)C(=O)C32CC2)CC1] | 131 |
| 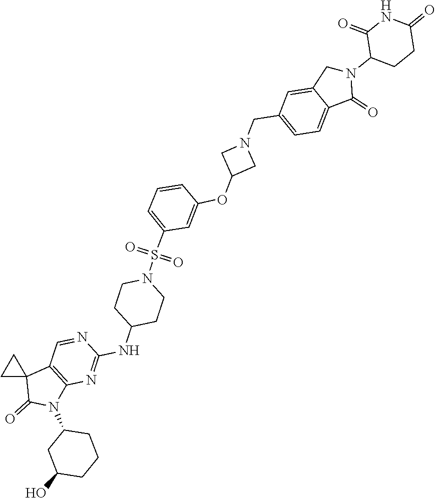 [SMILES: O=C1CCC(N2Cc3cc(CN4CC(Oc5cccc(S(=O)(=O)N6CCC(Nc7ncc8c(n7)N([C@@H]7CCC[C@@H](O)C7)C(=O)C87CC7)CC6)c5)C4)ccc3C2=O)C(=O)N1] | 132 |
| 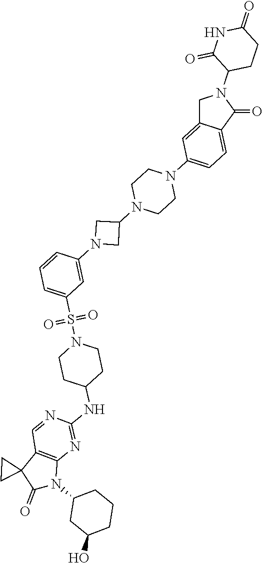 [SMILES: O=C1CCC(N2Cc3cc(N4CCN(C5CN(c6cccc(S(=O)(=O)N7CCC(Nc8ncc9c(n8)N([C@@H]8CCC[C@@H](O)C8)C(=O)C98CC8)CC7)c6)C5)CC4)ccc3C2=O)C(=O)N1] | 133 |
| 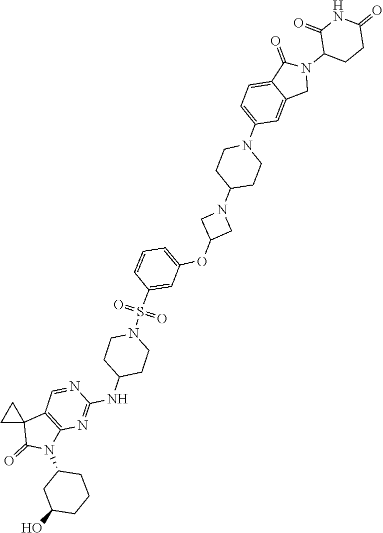 [SMILES: O=C1CCC(N2Cc3cc(N4CCC(N5CC(Oc6cccc(S(=O)(=O)N7CCC(Nc8ncc9c(n8)N([C@@H]8CCC[C@@H](O)C8)C(=O)C98CC8)CC7)c6)C5)CC4)ccc3C2=O)C(=O)N1] | 134 |
| 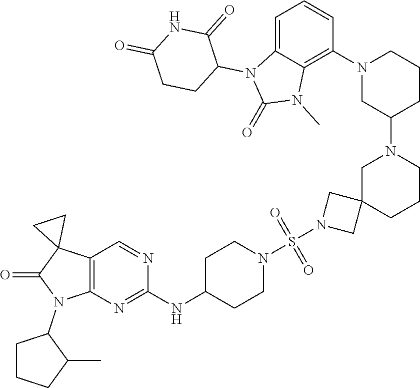 [SMILES: CC1CCCC1N1C(=O)C2(CC2)c2cnc(NC3CCN(S(=O)(=O)N4CC5(CCCN(C6CCCN(c7cccc8c7n(C)c(=O)n8C7CCC(=O)NC7=O)C6)C5)C4)CC3)nc21] | 135 |
| 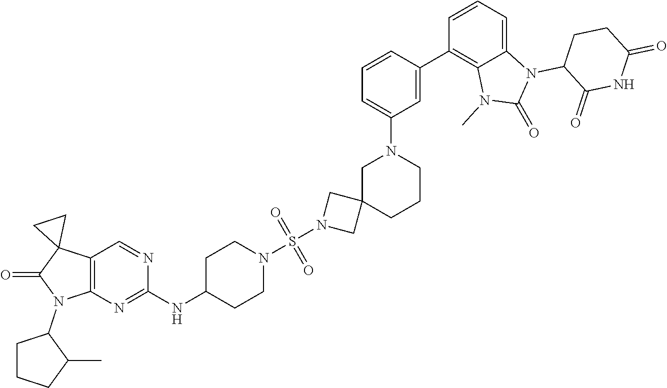 [SMILES: CC1CCCC1N1C(=O)C2(CC2)c2cnc(NC3CCN(S(=O)(=O)N4CC5(CCCN(c6cccc(-c7cccc8c7n(C)c(=O)n8C7CCC(=O)NC7=O)c6)C5)C4)CC3)nc21] | 136 |
| 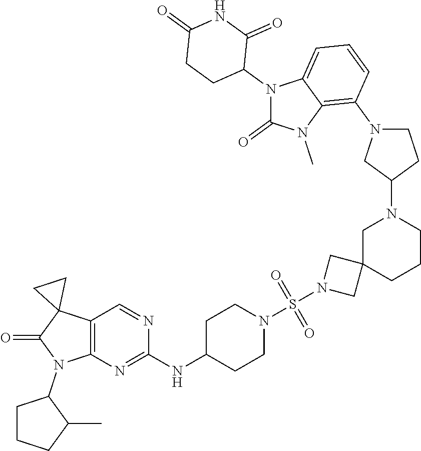 [SMILES: CC1CCCC1N1C(=O)C2(CC2)c2cnc(NC3CCN(S(=O)(=O)N4CC5(CCCN(C6CCN(c7cccc8c7n(C)c(=O)n8C7CCC(=O)NC7=O)C6)C5)C4)CC3)nc21] | 137 |
| 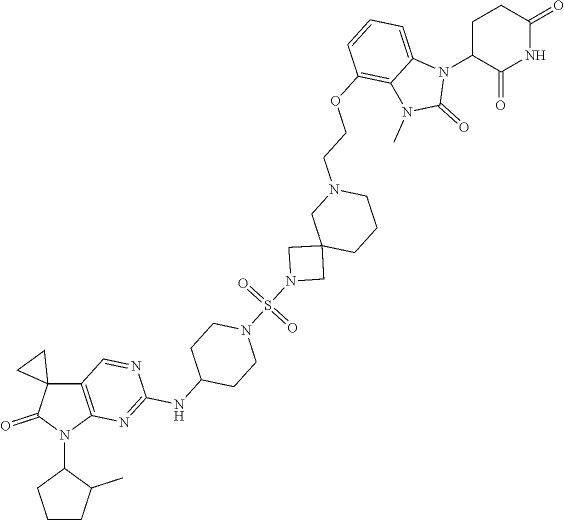 [SMILES: CC1CCCC1N1C(=O)C2(CC2)c2cnc(NC3CCN(S(=O)(=O)N4CC5(CCCN(CCOc6cccc7c6n(C)c(=O)n7C6CCC(=O)NC6=O)C5)C4)CC3)nc21] | 138 |
| 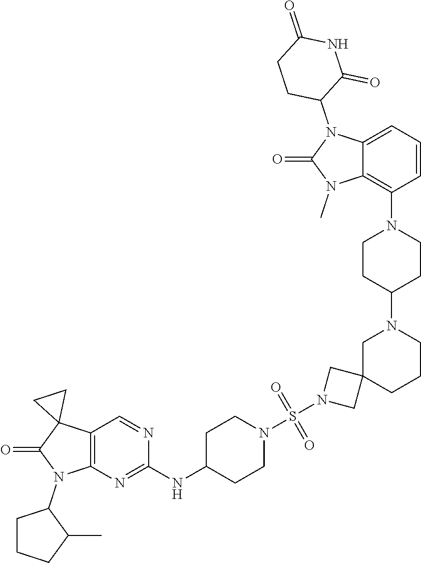 [SMILES: CC1CCCC1N1C(=O)C2(CC2)c2cnc(NC3CCN(S(=O)(=O)N4CC5(CCCN(C6CCN(c7cccc8c7n(C)c(=O)n8C7CCC(=O)NC7=O)CC6)C5)C4)CC3)nc21] | 139 |
| 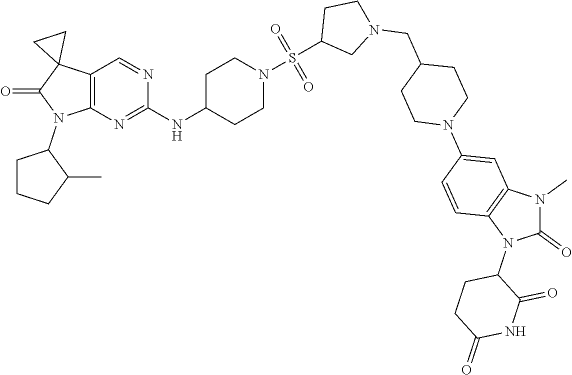 [SMILES: CC1CCCC1N1C(=O)C2(CC2)c2cnc(NC3CCN(S(=O)(=O)C4CCN(CC5CCN(c6ccc7c(c6)n(C)c(=O)n7C6CCC(=O)NC6=O)CC5)C4)CC3)nc21] | 140 |
| 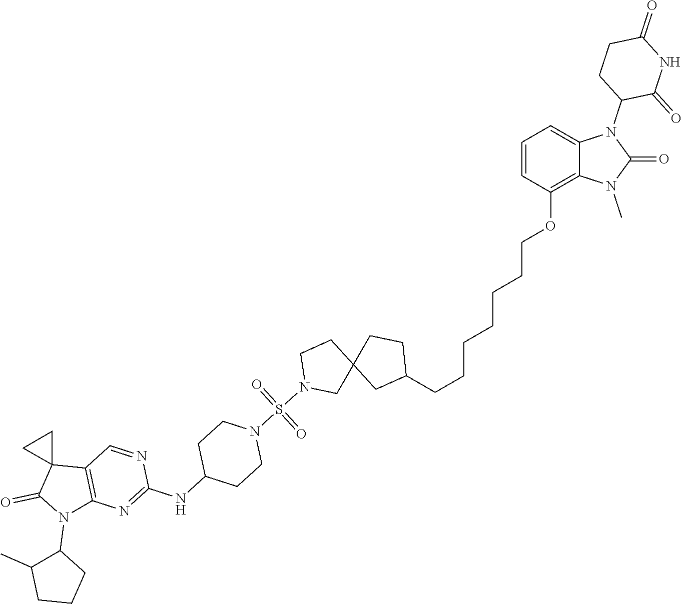 [SMILES: CC1CCCC1N1C(=O)C2(CC2)c2cnc(NC3CCN(S(=O)(=O)N4CCC5(CCC(CCCCCCCOc6cccc7c6n(C)c(=O)n7C6CCC(=O)NC6=O)C5)C4)CC3)nc21] | 141 |
| 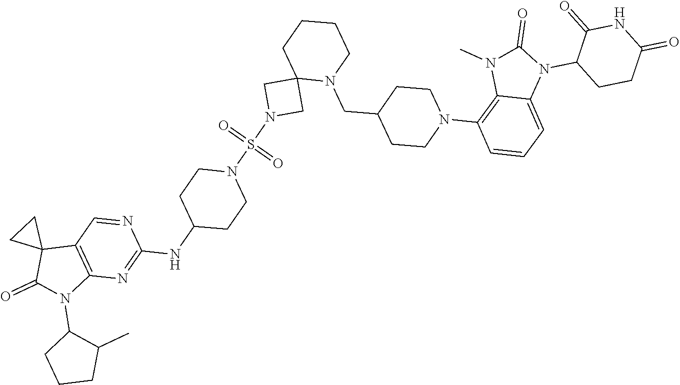 [SMILES: CC1CCCC1N1C(=O)C2(CC2)c2cnc(NC3CCN(S(=O)(=O)N4CC5(CCCCN5CC5CCN(c6cccc7c6n(C)c(=O)n7C6CCC(=O)NC6=O)CC5)C4)CC3)nc21] | 142 |
| 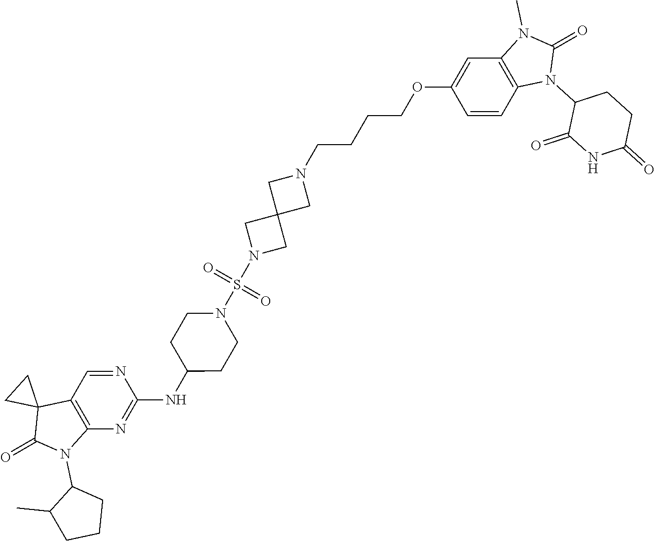 [SMILES: CC1CCCC1N1C(=O)C2(CC2)c2cnc(NC3CCN(S(=O)(=O)N4CC5(CN(CCCCOc6ccc7c(c6)n(C)c(=O)n7C6CCC(=O)NC6=O)C5)C4)CC3)nc21] | 143 |
| 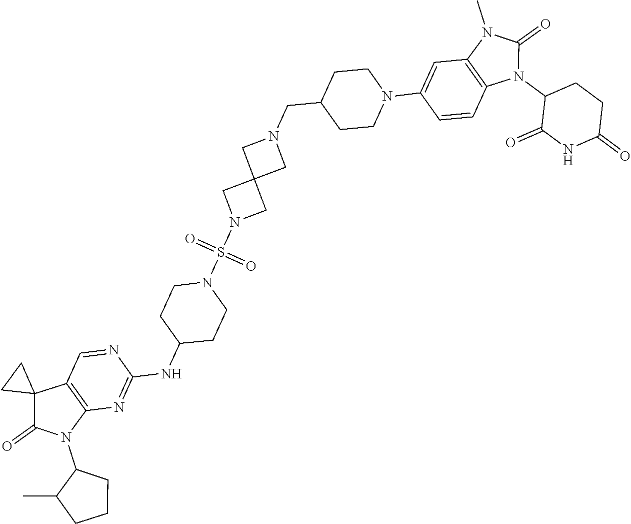 [SMILES: CC1CCCC1N1C(=O)C2(CC2)c2cnc(NC3CCN(S(=O)(=O)N4CC5(CN(CC6CCN(c7ccc8c(c7)n(C)c(=O)n8C7CCC(=O)NC7=O)CC6)C5)C4)CC3)nc21] | 144 |
| 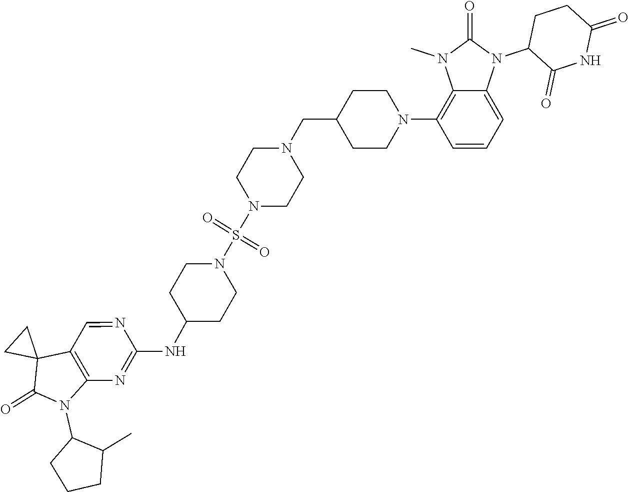 [SMILES: CC1CCCC1N1C(=O)C2(CC2)c2cnc(NC3CCN(S(=O)(=O)N4CCN(CC5CCN(c6cccc7c6n(C)c(=O)n7C6CCC(=O)NC6=O)CC5)CC4)CC3)nc21] | 145 |
| 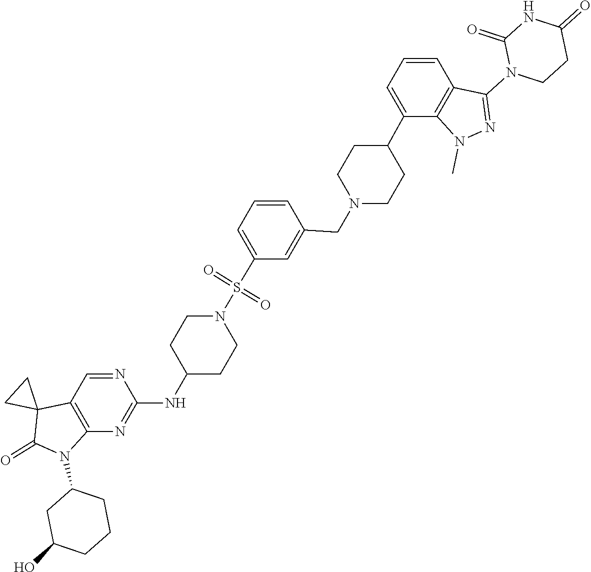 [SMILES: Cn1nc(N2CCC(=O)NC2=O)c2cccc(C3CCN(Cc4cccc(S(=O)(=O)N5CCC(Nc6ncc7c(n6)N([C@@H]6CCC[C@@H](O)C6)C(=O)C76CC6)CC5)c4)CC3)c21] | 146 |
| 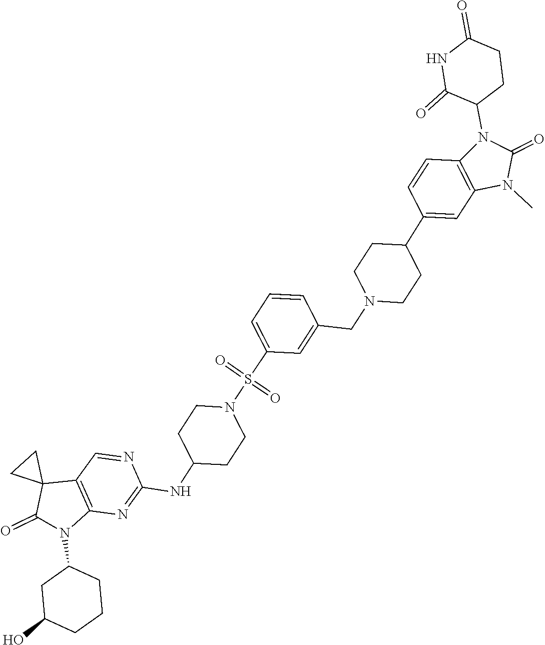 [SMILES: Cn1c(=O)n(C2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)c4)CC3)cc21] | 147 |
| 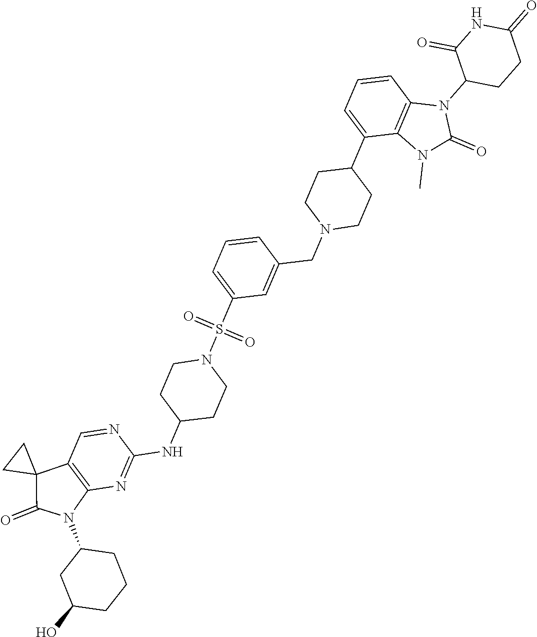 [SMILES: Cn1c(=O)n(C2CCC(=O)NC2=O)c2cccc(C3CCN(Cc4cccc(S(=O)(=O)N5CCC(Nc6ncc7c(n6)N([C@@H]6CCC[C@@H](O)C6)C(=O)C76CC6)CC5)c4)CC3)c21] | 148 |
| 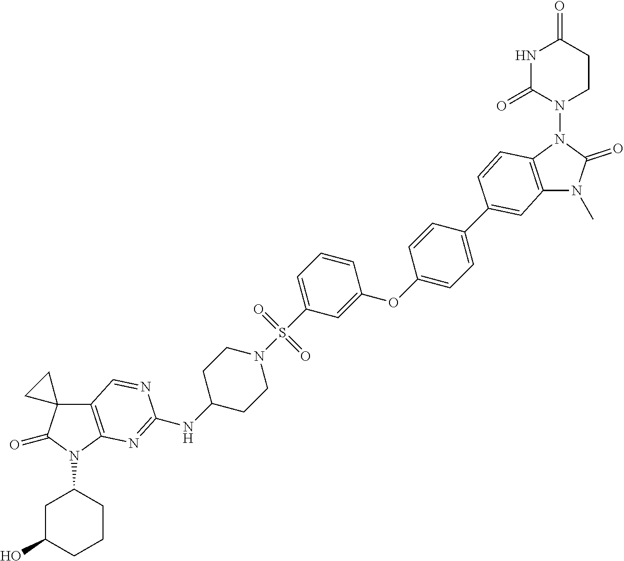 [SMILES: Cn1c(=O)n(N2CCC(=O)NC2=O)c2ccc(-c3ccc(Oc4cccc(S(=O)(=O)N5CCC(Nc6ncc7c(n6)N([C@@H]6CCC[C@@H](O)C6)C(=O)C76CC6)CC5)c4)cc3)cc21] | 149 |
| 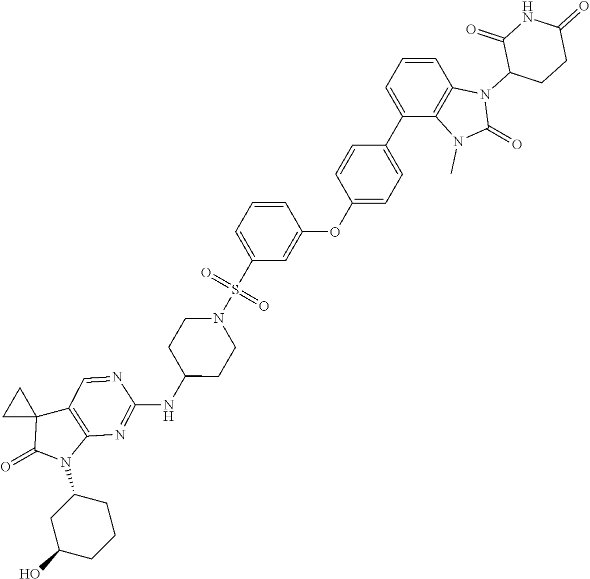 [SMILES: Cn1c(=O)n(C2CCC(=O)NC2=O)c2cccc(-c3ccc(Oc4cccc(S(=O)(=O)N5CCC(Nc6ncc7c(n6)N([C@@H]6CCC[C@@H](O)C6)C(=O)C76CC6)CC5)c4)cc3)c21] | 150 |
| 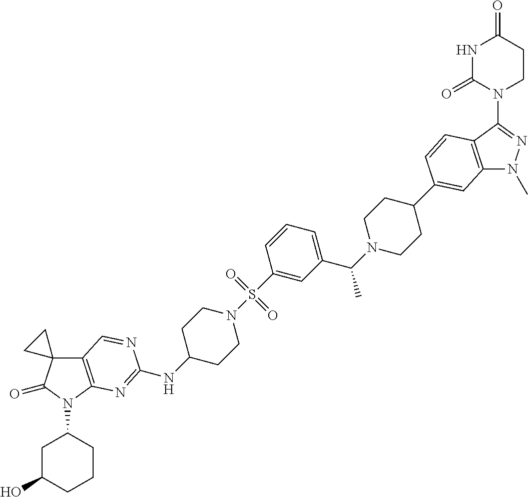 [SMILES: C[C@H](c1cccc(S(=O)(=O)N2CCC(Nc3ncc4c(n3)N([C@@H]3CCC[C@@H](O)C3)C(=O)C43CC3)CC2)c1)N1CCC(c2ccc3c(N4CCC(=O)NC4=O)nn(C)c3c2)CC1] | 151 |
| 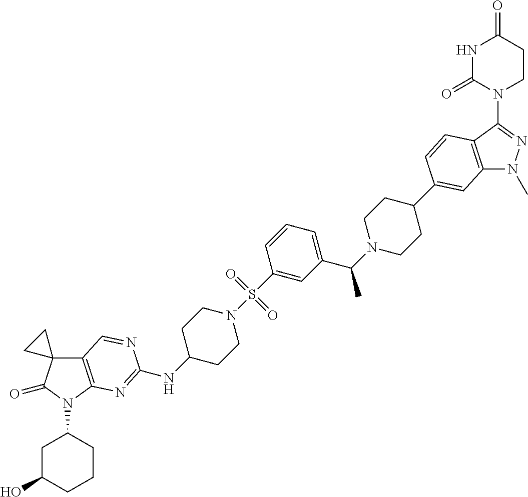 [SMILES: C[C@@H](c1cccc(S(=O)(=O)N2CCC(Nc3ncc4c(n3)N([C@@H]3CCC[C@@H](O)C3)C(=O)C43CC3)CC2)c1)N1CCC(c2ccc3c(N4CCC(=O)NC4=O)nn(C)c3c2)CC1] | 152 |
| 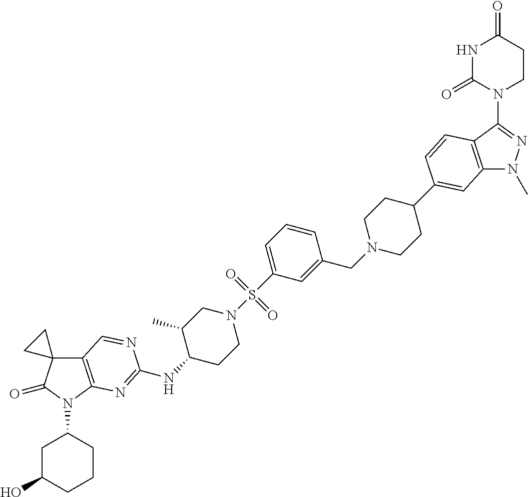 [SMILES: C[C@@H]1CN(S(=O)(=O)c2cccc(CN3CCC(c4ccc5c(N6CCC(=O)NC6=O)nn(C)c5c4)CC3)c2)CC[C@@H]1Nc1ncc2c(n1)N([C@@H]1CCC[C@@H](O)C1)C(=O)C21CC1] | 153 |
| 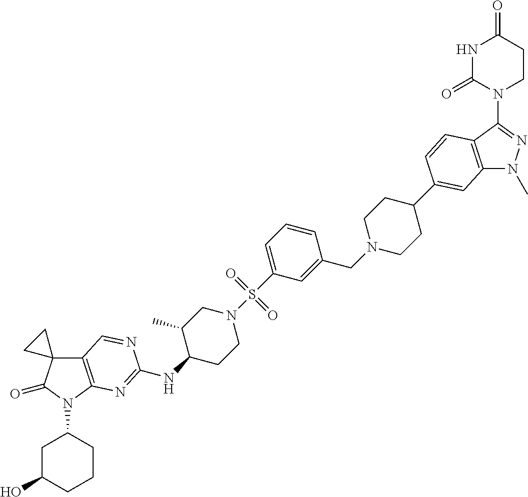 [SMILES: C[C@@H]1CN(S(=O)(=O)c2cccc(CN3CCC(c4ccc5c(N6CCC(=O)NC6=O)nn(C)c5c4)CC3)c2)CC[C@H]1Nc1ncc2c(n1)N([C@@H]1CCC[C@@H](O)C1)C(=O)C21CC1] | 154 |
| 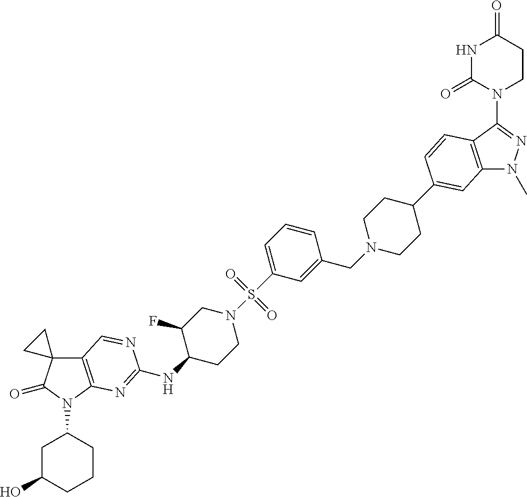 [SMILES: Cn1nc(N2CCC(=O)NC2=O)c2ccc(C3CCN(Cc4cccc(S(=O)(=O)N5CC[C@@H](Nc6ncc7c(n6)N([C@@H]6CCC[C@@H](O)C6)C(=O)C76CC6)[C@@H](F)C5)c4)CC3)cc21] | 155 |
| 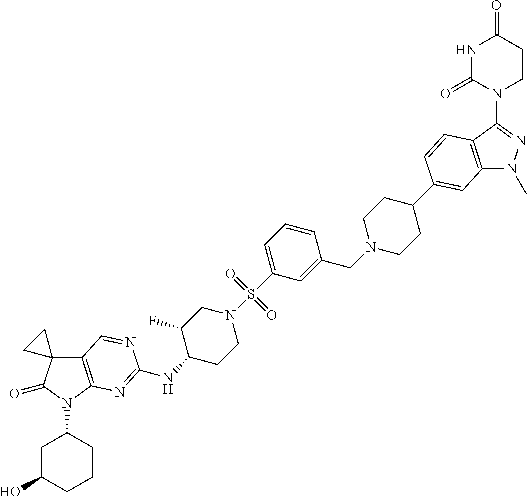 [SMILES: Cn1nc(N2CCC(=O)NC2=O)c2ccc(C3CCN(Cc4cccc(S(=O)(=O)N5CC[C@H](Nc6ncc7c(n6)N([C@@H]6CCC[C@@H](O)C6)C(=O)C76CC6)[C@H](F)C5)c4)CC3)cc21] | 156 |
| 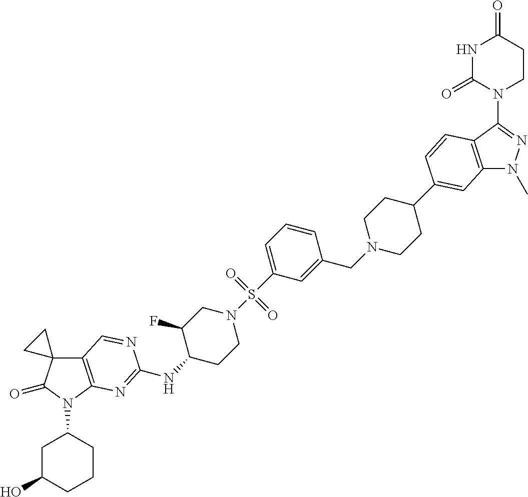 [SMILES: Cn1nc(N2CCC(=O)NC2=O)c2ccc(C3CCN(Cc4cccc(S(=O)(=O)N5CC[C@H](Nc6ncc7c(n6)N([C@@H]6CCC[C@@H](O)C6)C(=O)C76CC6)[C@@H](F)C5)c4)CC3)cc21] | 157 |
| 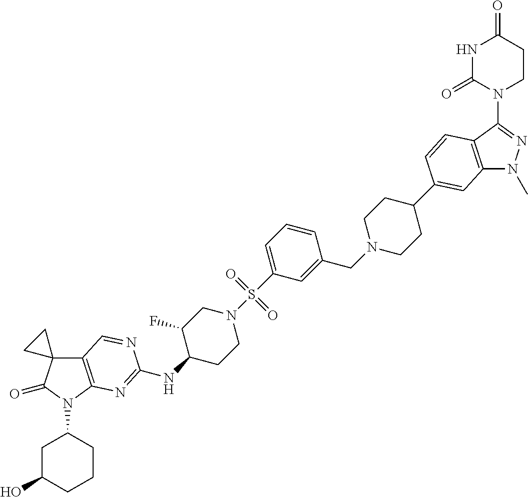 [SMILES: Cn1nc(N2CCC(=O)NC2=O)c2ccc(C3CCN(Cc4cccc(S(=O)(=O)N5CC[C@@H](Nc6ncc7c(n6)N([C@@H]6CCC[C@@H](O)C6)C(=O)C76CC6)[C@H](F)C5)c4)CC3)cc21] | 158 |
| 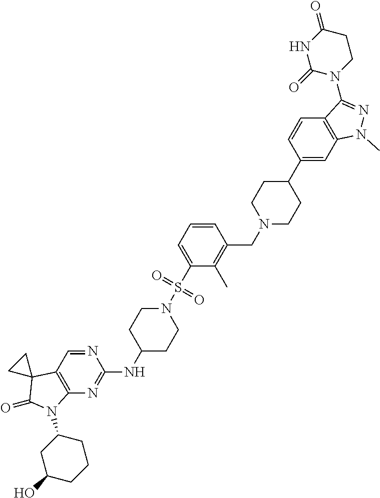 [SMILES: Cc1c(CN2CCC(c3ccc4c(N5CCC(=O)NC5=O)nn(C)c4c3)CC2)cccc1S(=O)(=O)N1CCC(Nc2ncc3c(n2)N([C@@H]2CCC[C@@H](O)C2)C(=O)C32CC2)CC1] | 159 |
| 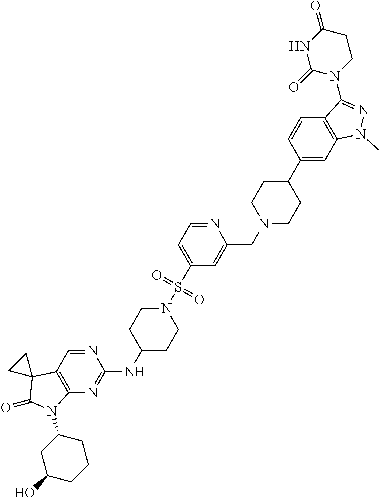 [SMILES: Cn1nc(N2CCC(=O)NC2=O)c2ccc(C3CCN(Cc4cc(S(=O)(=O)N5CCC(Nc6ncc7c(n6)N([C@@H]6CCC[C@@H](O)C6)C(=O)C76CC6)CC5)ccn4)CC3)cc21] | 160 |
| 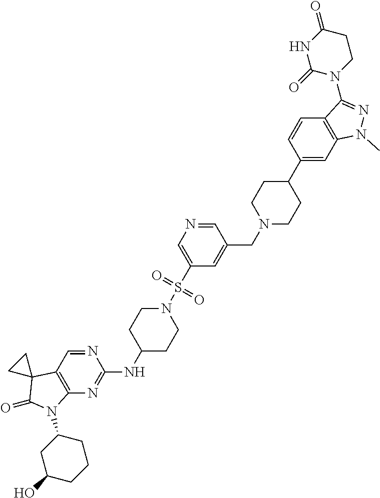 [SMILES: Cn1nc(N2CCC(=O)NC2=O)c2ccc(C3CCN(Cc4cncc(S(=O)(=O)N5CCC(Nc6ncc7c(n6)N([C@@H]6CCC[C@@H](O)C6)C(=O)C76CC6)CC5)c4)CC3)cc21] | 161 |
| 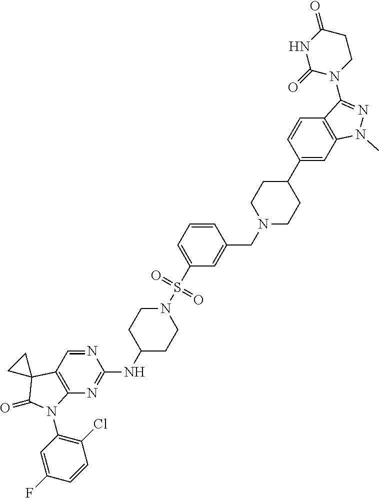 [SMILES: Cn1nc(N2CCC(=O)NC2=O)c2ccc(C3CCN(Cc4cccc(S(=O)(=O)N5CCC(Nc6ncc7c(n6)N(c6cc(F)ccc6Cl)C(=O)C76CC6)CC5)c4)CC3)cc21] | 162 |
| 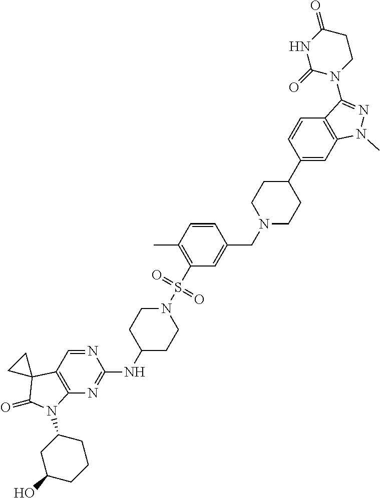 [SMILES: Cc1ccc(CN2CCC(c3ccc4c(N5CCC(=O)NC5=O)nn(C)c4c3)CC2)cc1S(=O)(=O)N1CCC(Nc2ncc3c(n2)N([C@@H]2CCC[C@@H](O)C2)C(=O)C32CC2)CC1] | 163 |
| 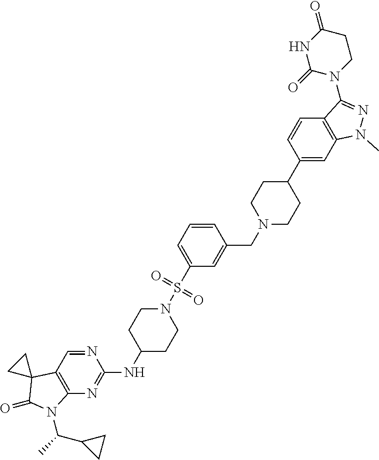 [SMILES: C[C@@H](C1CC1)N1C(=O)C2(CC2)c2cnc(NC3CCN(S(=O)(=O)c4cccc(CN5CCC(c6ccc7c(N8CCC(=O)NC8=O)nn(C)c7c6)CC5)c4)CC3)nc21] | 164 |
| 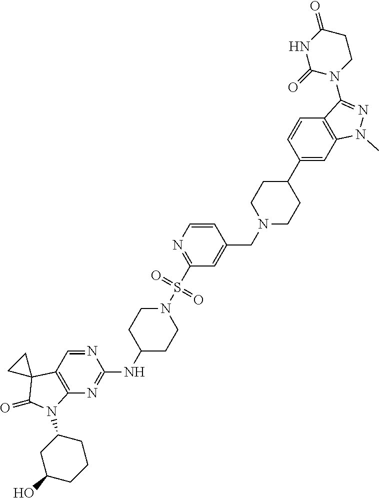 [SMILES: Cn1nc(N2CCC(=O)NC2=O)c2ccc(C3CCN(Cc4ccnc(S(=O)(=O)N5CCC(Nc6ncc7c(n6)N([C@@H]6CCC[C@@H](O)C6)C(=O)C76CC6)CC5)c4)CC3)cc21] | 165 |
| 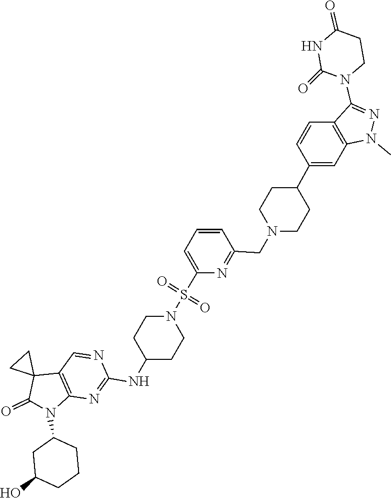 [SMILES: Cn1nc(N2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)n4)CC3)cc21] | 166 |
| 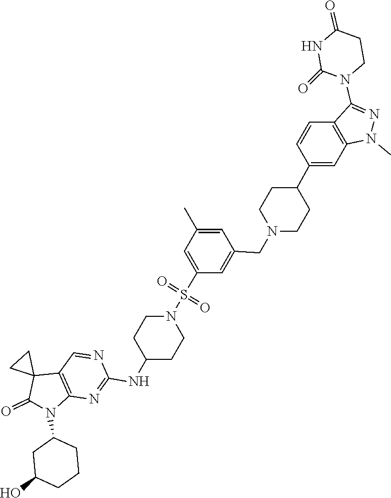 [SMILES: Cc1cc(CN2CCC(c3ccc4c(N5CCC(=O)NC5=O)nn(C)c4c3)CC2)cc(S(=O)(=O)N2CCC(Nc3ncc4c(n3)N([C@@H]3CCC[C@@H](O)C3)C(=O)C43CC3)CC2)c1] | 167 |
| 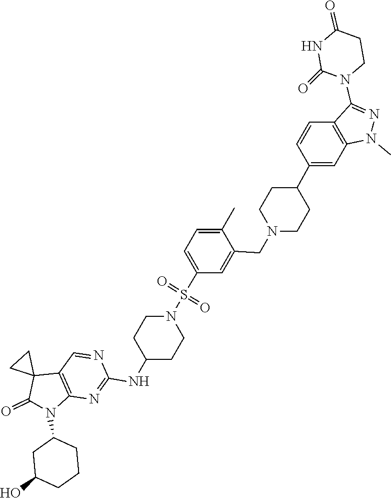 [SMILES: Cc1ccc(S(=O)(=O)N2CCC(Nc3ncc4c(n3)N([C@@H]3CCC[C@@H](O)C3)C(=O)C43CC3)CC2)cc1CN1CCC(c2ccc3c(N4CCC(=O)NC4=O)nn(C)c3c2)CC1] | 168 |
| 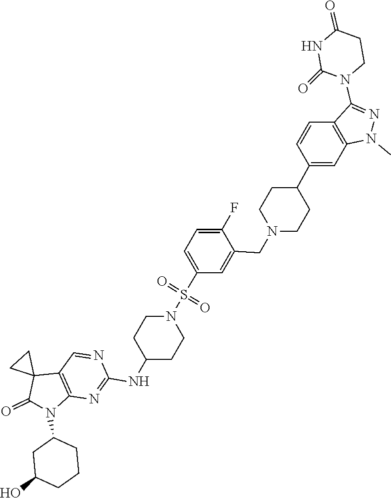 [SMILES: Cn1nc(N2CCC(=O)NC2=O)c2ccc(C3CCN(Cc4cc(S(=O)(=O)N5CCC(Nc6ncc7c(n6)N([C@@H]6CCC[C@@H](O)C6)C(=O)C76CC6)CC5)ccc4F)CC3)cc21] | 169 |
| 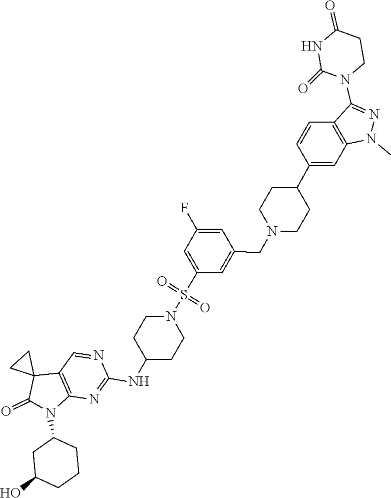 [SMILES: Cn1nc(N2CCC(=O)NC2=O)c2ccc(C3CCN(Cc4cc(F)cc(S(=O)(=O)N5CCC(Nc6ncc7c(n6)N([C@@H]6CCC[C@@H](O)C6)C(=O)C76CC6)CC5)c4)CC3)cc21] | 170 |
| 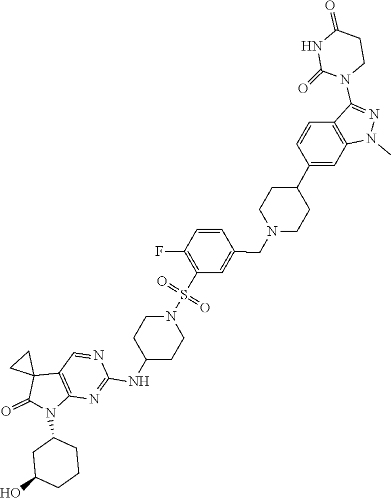 [SMILES: Cn1nc(N2CCC(=O)NC2=O)c2ccc(C3CCN(Cc4ccc(F)c(S(=O)(=O)N5CCC(Nc6ncc7c(n6)N([C@@H]6CCC[C@@H](O)C6)C(=O)C76CC6)CC5)c4)CC3)cc21] | 171 |
| 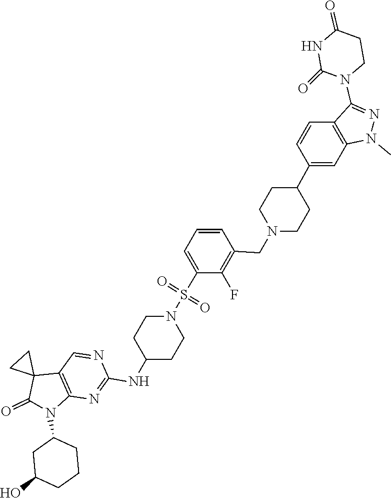 [SMILES: Cn1nc(N2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)c4F)CC3)cc21] | 172 |
| 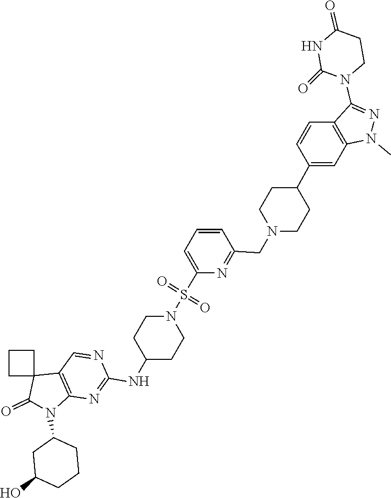 [SMILES: Cn1nc(N2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CCC6)CC5)n4)CC3)cc21] | 174 |
| 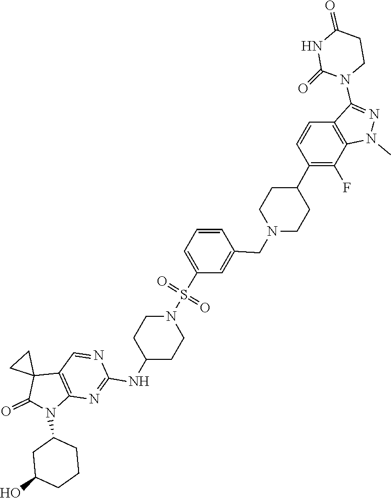 [SMILES: Cn1nc(N2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)c4)CC3)c(F)c21] | 175 |
| 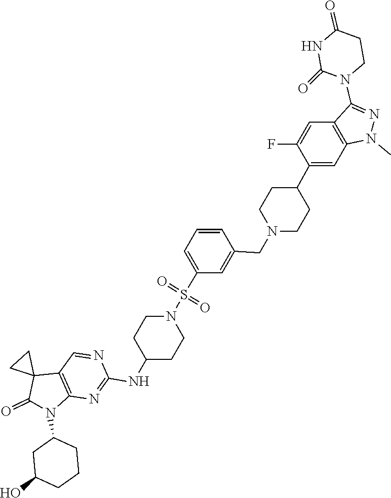 [SMILES: Cn1nc(N2CCC(=O)NC2=O)c2cc(F)c(C3CCN(Cc4cccc(S(=O)(=O)N5CCC(Nc6ncc7c(n6)N([C@@H]6CCC[C@@H](O)C6)C(=O)C76CC6)CC5)c4)CC3)cc21] | 176 |
| 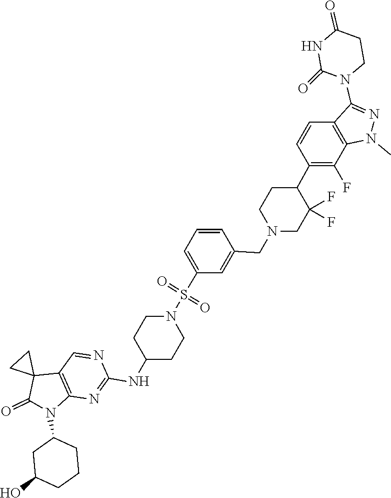 [SMILES: Cn1nc(N2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)c4)CC3(F)F)c(F)c21] | 177 |
| 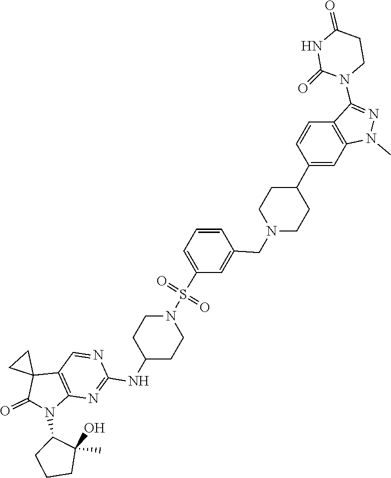 [SMILES: Cn1nc(N2CCC(=O)NC2=O)c2ccc(C3CCN(Cc4cccc(S(=O)(=O)N5CCC(Nc6ncc7c(n6)N([C@H]6CCC[C@]6(C)O)C(=O)C76CC6)CC5)c4)CC3)cc21] | 178 |
| 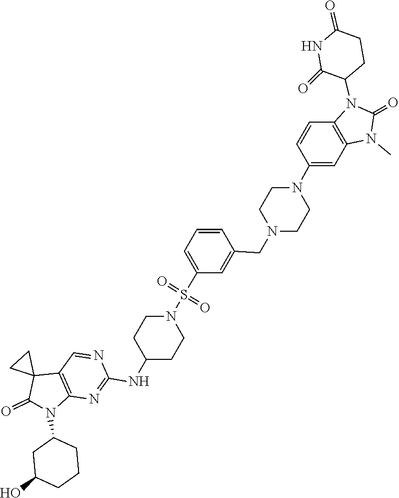 [SMILES: Cn1c(=O)n(C2CCC(=O)NC2=O)c2ccc(N3CCN(Cc4cccc(S(=O)(=O)N5CCC(Nc6ncc7c(n6)N([C@@H]6CCC[C@@H](O)C6)C(=O)C76CC6)CC5)c4)CC3)cc21] | 179 |
| 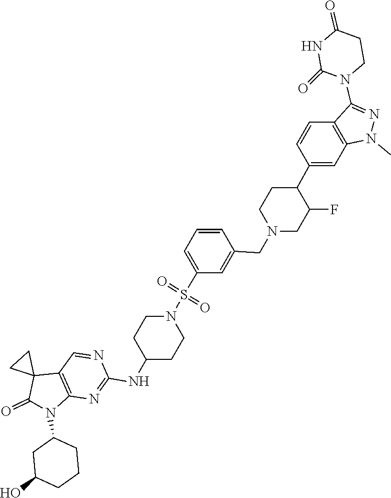 [SMILES: Cn1nc(N2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)c4)CC3F)cc21] | 180 |
| 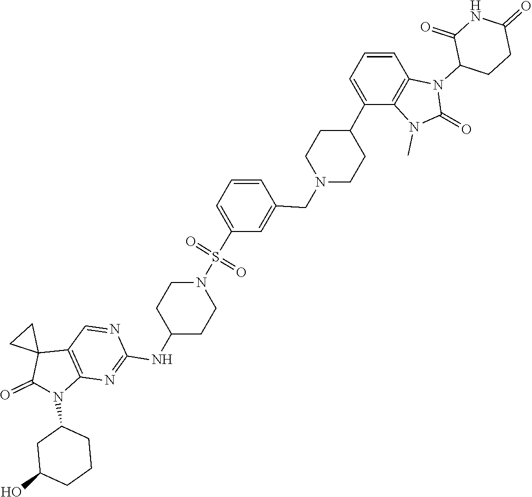 [SMILES: Cn1c(=O)n(C2CCC(=O)NC2=O)c2cccc(C3CCN(Cc4cccc(S(=O)(=O)N5CCC(Nc6ncc7c(n6)N([C@@H]6CCC[C@@H](O)C6)C(=O)C76CC6)CC5)c4)CC3)c21] | 181 |
| 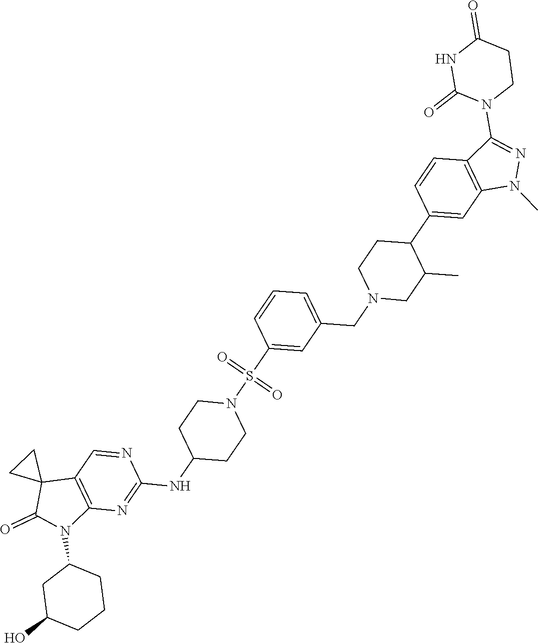 [SMILES: CC1CN(Cc2cccc(S(=O)(=O)N3CCC(Nc4ncc5c(n4)N([C@@H]4CCC[C@@H](O)C4)C(=O)C54CC4)CC3)c2)CCC1c1ccc2c(N3CCC(=O)NC3=O)nn(C)c2c1] | 182 |
| 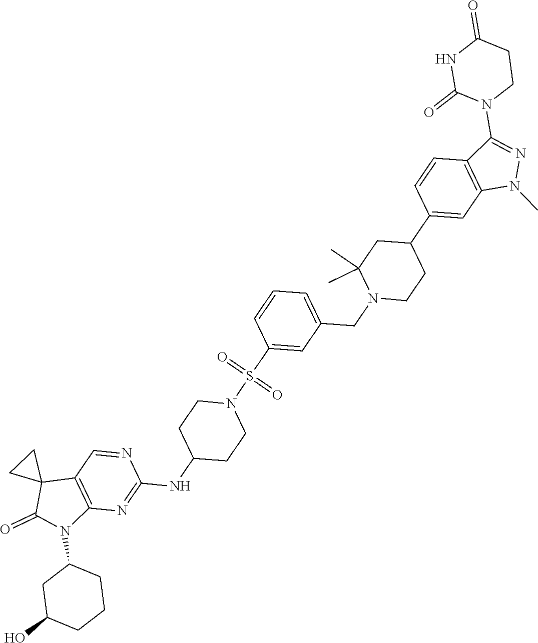 [SMILES: Cn1nc(N2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)c4)C(C)(C)C3)cc21] | 183 |
| 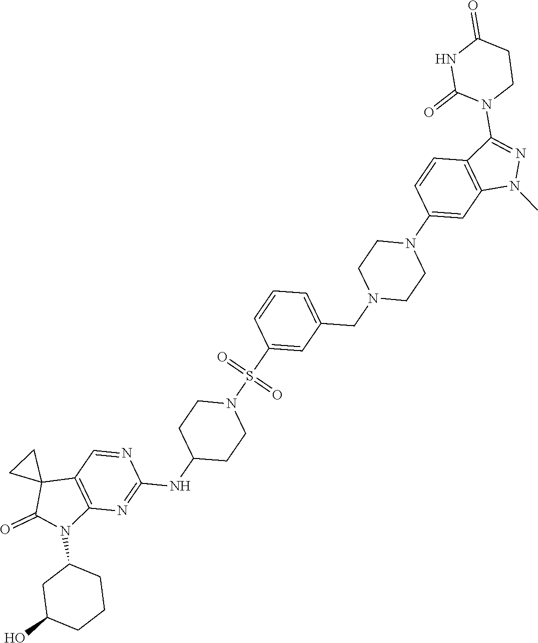 [SMILES: Cn1nc(N2CCC(=O)NC2=O)c2ccc(N3CCN(Cc4cccc(S(=O)(=O)N5CCC(Nc6ncc7c(n6)N([C@@H]6CCC[C@@H](O)C6)C(=O)C76CC6)CC5)c4)CC3)cc21] | 184 |
| 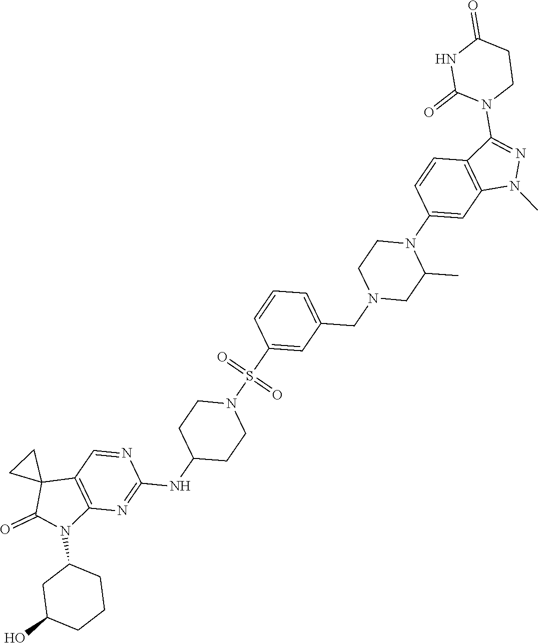 [SMILES: CC1CN(Cc2cccc(S(=O)(=O)N3CCC(Nc4ncc5c(n4)N([C@@H]4CCC[C@@H](O)C4)C(=O)C54CC4)CC3)c2)CCN1c1ccc2c(N3CCC(=O)NC3=O)nn(C)c2c1] | 185 |
| 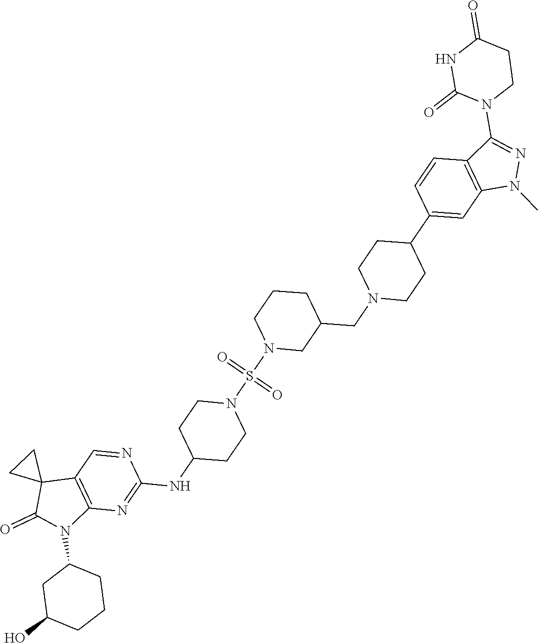 [SMILES: Cn1nc(N2CCC(=O)NC2=O)c2ccc(C3CCN(CC4CCCN(S(=O)(=O)N5CCC(Nc6ncc7c(n6)N([C@@H]6CCC[C@@H](O)C6)C(=O)C76CC6)CC5)C4)CC3)cc21] | 186 |
| 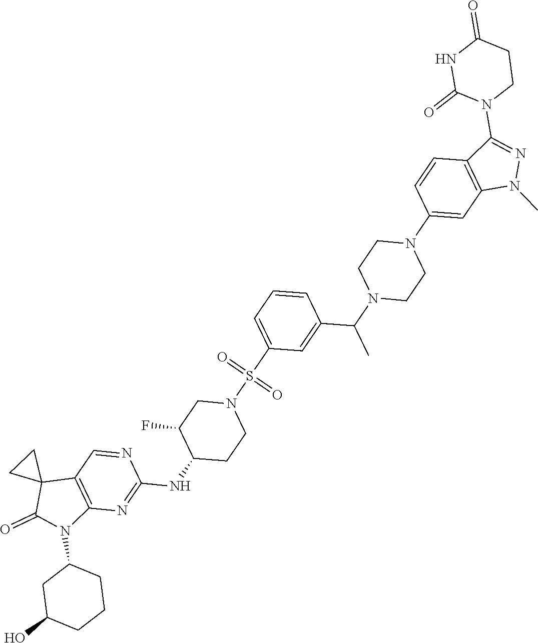 [SMILES: CC(c1cccc(S(=O)(=O)N2CC[C@H](Nc3ncc4c(n3)N([C@@H]3CCC[C@@H](O)C3)C(=O)C43CC3)[C@H](F)C2)c1)N1CCN(c2ccc3c(N4CCC(=O)NC4=O)nn(C)c3c2)CC1] | 187 |
| 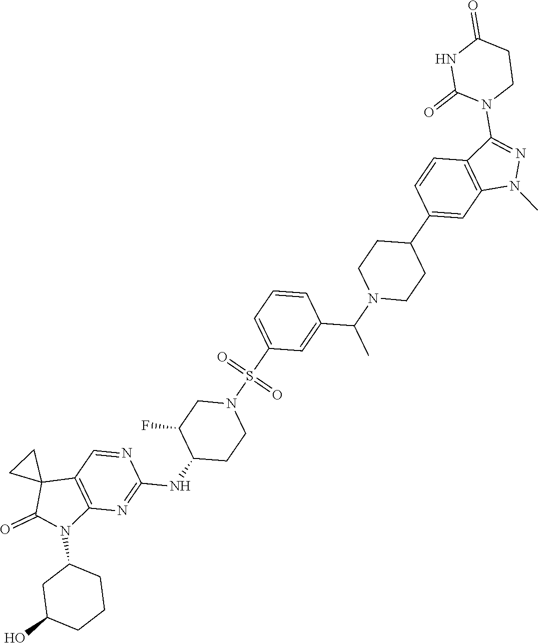 [SMILES: CC(c1cccc(S(=O)(=O)N2CC[C@H](Nc3ncc4c(n3)N([C@@H]3CCC[C@@H](O)C3)C(=O)C43CC3)[C@H](F)C2)c1)N1CCC(c2ccc3c(N4CCC(=O)NC4=O)nn(C)c3c2)CC1] | 188 |
| 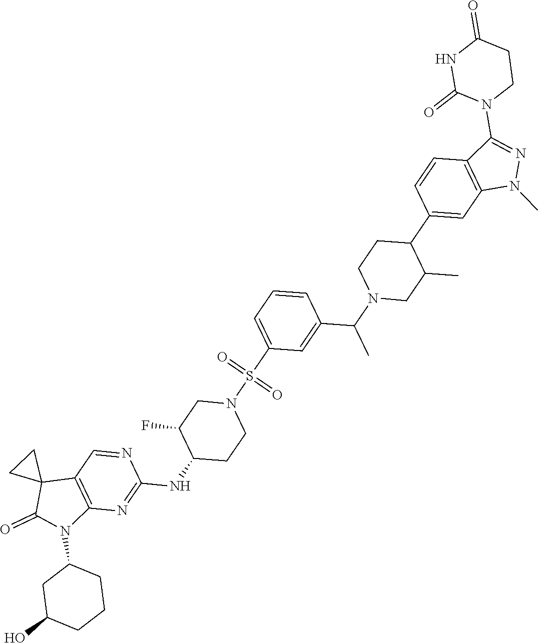 [SMILES: CC1CN(C(C)c2cccc(S(=O)(=O)N3CC[C@H](Nc4ncc5c(n4)N([C@@H]4CCC[C@@H](O)C4)C(=O)C54CC4)[C@H](F)C3)c2)CCC1c1ccc2c(N3CCC(=O)NC3=O)nn(C)c2c1] | 189 |
| 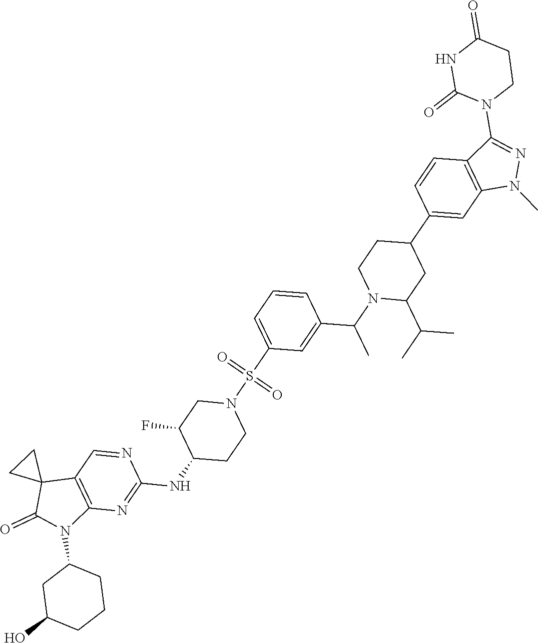 [SMILES: CC(C)C1CC(c2ccc3c(N4CCC(=O)NC4=O)nn(C)c3c2)CCN1C(C)c1cccc(S(=O)(=O)N2CC[C@H](Nc3ncc4c(n3)N([C@@H]3CCC[C@@H](O)C3)C(=O)C43CC3)[C@H](F)C2)c1] | 190 |
| 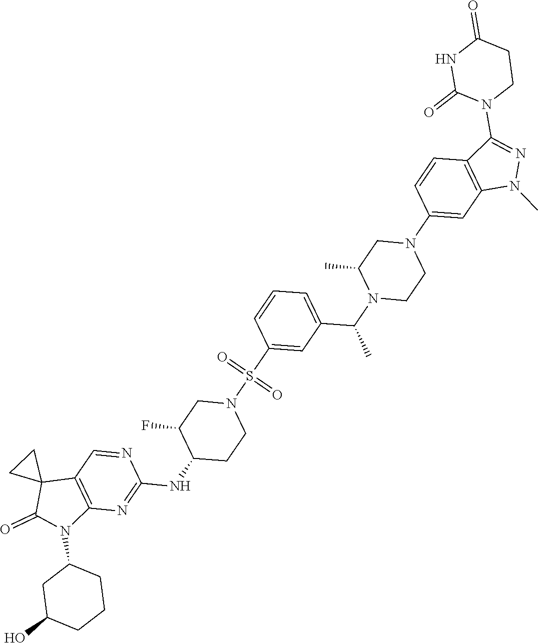 [SMILES: C[C@@H]1CN(c2ccc3c(N4CCC(=O)NC4=O)nn(C)c3c2)CCN1[C@H](C)c1cccc(S(=O)(=O)N2CC[C@H](Nc3ncc4c(n3)N([C@@H]3CCC[C@@H](O)C3)C(=O)C43CC3)[C@H](F)C2)c1] | 191 |
| 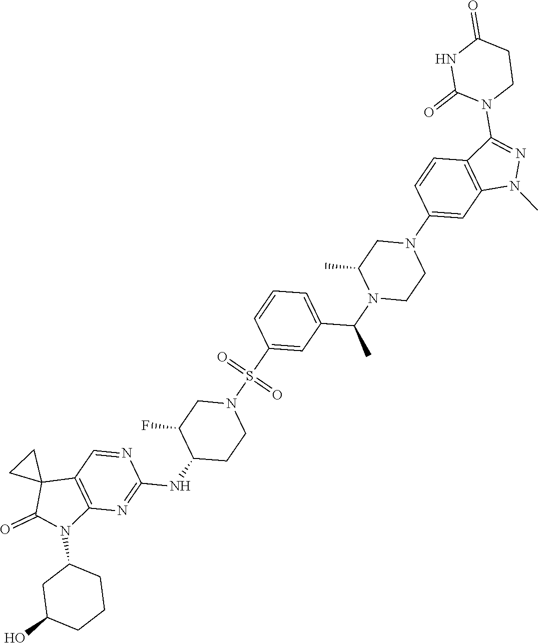 [SMILES: C[C@@H]1CN(c2ccc3c(N4CCC(=O)NC4=O)nn(C)c3c2)CCN1[C@@H](C)c1cccc(S(=O)(=O)N2CC[C@H](Nc3ncc4c(n3)N([C@@H]3CCC[C@@H](O)C3)C(=O)C43CC3)[C@H](F)C2)c1] | 192 |
| 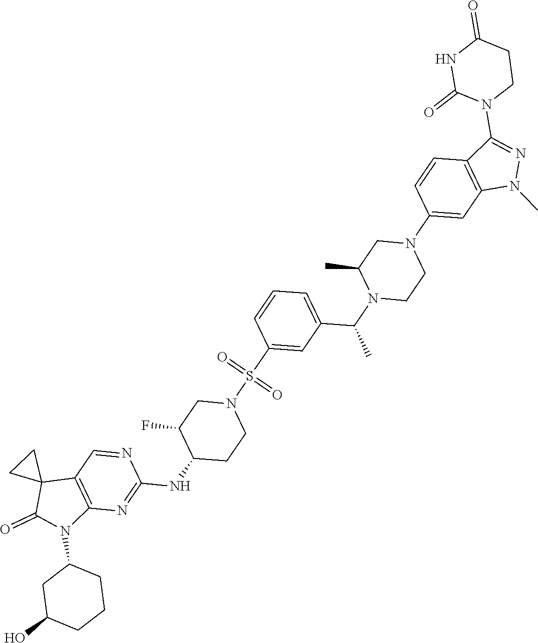 [SMILES: C[C@H](c1cccc(S(=O)(=O)N2CC[C@H](Nc3ncc4c(n3)N([C@@H]3CCC[C@@H](O)C3)C(=O)C43CC3)[C@H](F)C2)c1)N1CCN(c2ccc3c(N4CCC(=O)NC4=O)nn(C)c3c2)C[C@@H]1C] | 193 |
| 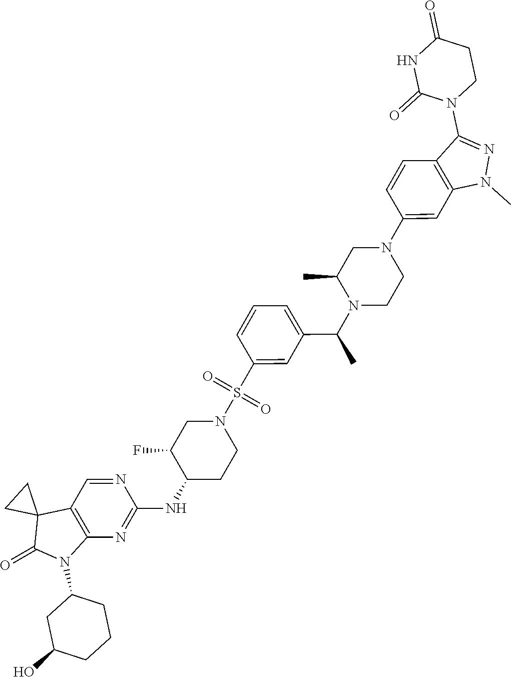 [SMILES: C[C@H]1CN(c2ccc3c(N4CCC(=O)NC4=O)nn(C)c3c2)CCN1[C@@H](C)c1cccc(S(=O)(=O)N2CC[C@H](Nc3ncc4c(n3)N([C@@H]3CCC[C@@H](O)C3)C(=O)C43CC3)[C@H](F)C2)c1] | 194 |
| 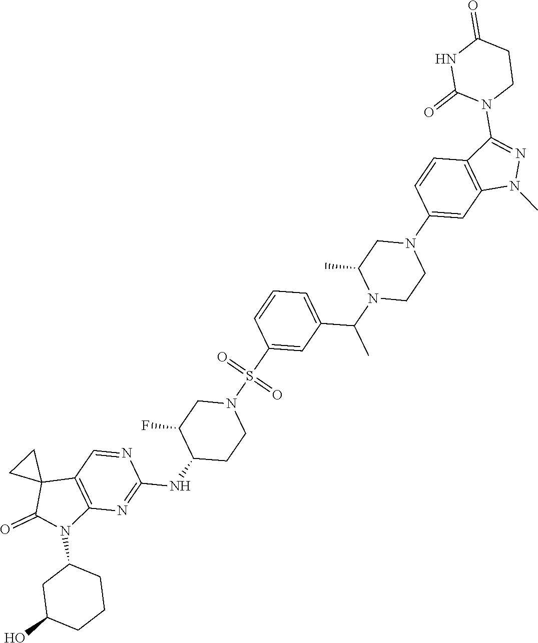 [SMILES: CC(c1cccc(S(=O)(=O)N2CC[C@H](Nc3ncc4c(n3)N([C@@H]3CCC[C@@H](O)C3)C(=O)C43CC3)[C@H](F)C2)c1)N1CCN(c2ccc3c(N4CCC(=O)NC4=O)nn(C)c3c2)C[C@H]1C] | 195 |
| 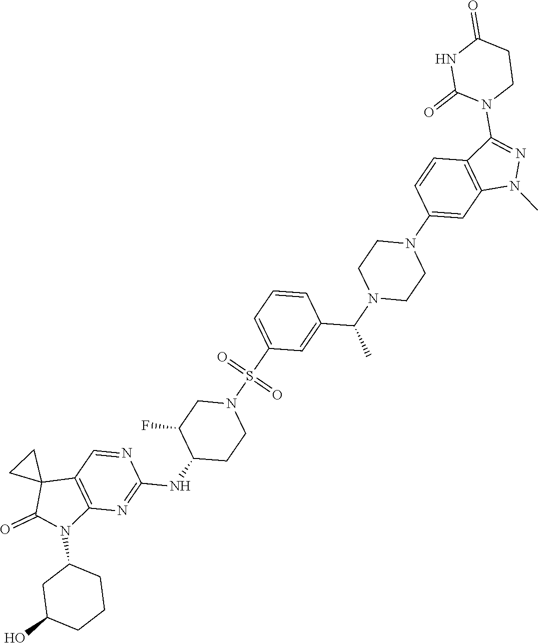 [SMILES: C[C@H](c1cccc(S(=O)(=O)N2CC[C@H](Nc3ncc4c(n3)N([C@@H]3CCC[C@@H](O)C3)C(=O)C43CC3)[C@H](F)C2)c1)N1CCN(c2ccc3c(N4CCC(=O)NC4=O)nn(C)c3c2)CC1] | 196 |
| 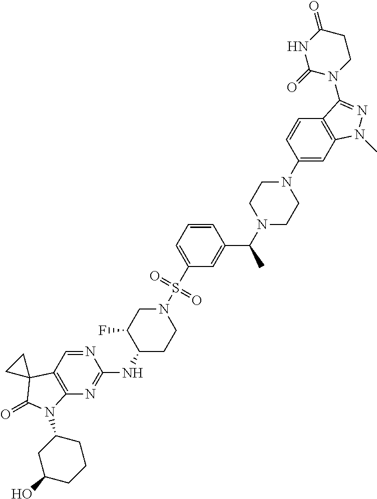 [SMILES: C[C@@H](c1cccc(S(=O)(=O)N2CC[C@H](Nc3ncc4c(n3)N([C@@H]3CCC[C@@H](O)C3)C(=O)C43CC3)[C@H](F)C2)c1)N1CCN(c2ccc3c(N4CCC(=O)NC4=O)nn(C)c3c2)CC1] | 197 |
| 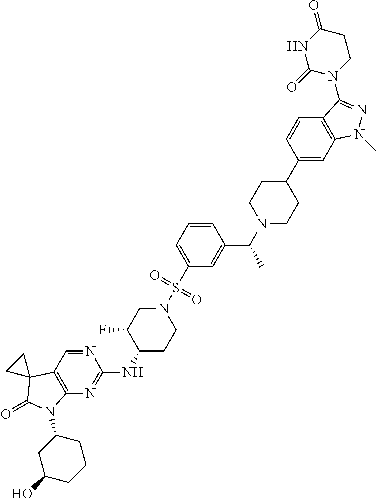 [SMILES: C[C@H](c1cccc(S(=O)(=O)N2CC[C@H](Nc3ncc4c(n3)N([C@@H]3CCC[C@@H](O)C3)C(=O)C43CC3)[C@H](F)C2)c1)N1CCC(c2ccc3c(N4CCC(=O)NC4=O)nn(C)c3c2)CC1] | 198 |
| 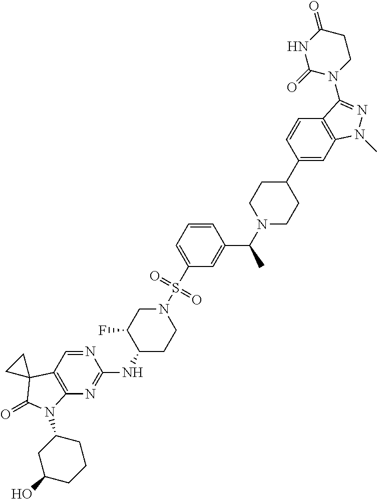 [SMILES: C[C@@H](c1cccc(S(=O)(=O)N2CC[C@H](Nc3ncc4c(n3)N([C@@H]3CCC[C@@H](O)C3)C(=O)C43CC3)[C@H](F)C2)c1)N1CCC(c2ccc3c(N4CCC(=O)NC4=O)nn(C)c3c2)CC1] | 199 |
| 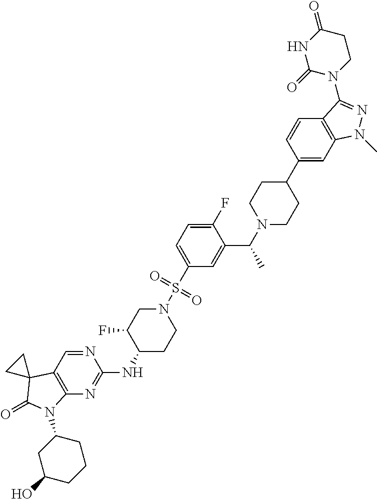 [SMILES: C[C@H](c1cc(S(=O)(=O)N2CC[C@H](Nc3ncc4c(n3)N([C@@H]3CCC[C@@H](O)C3)C(=O)C43CC3)[C@H](F)C2)ccc1F)N1CCC(c2ccc3c(N4CCC(=O)NC4=O)nn(C)c3c2)CC1] | 200 |
| 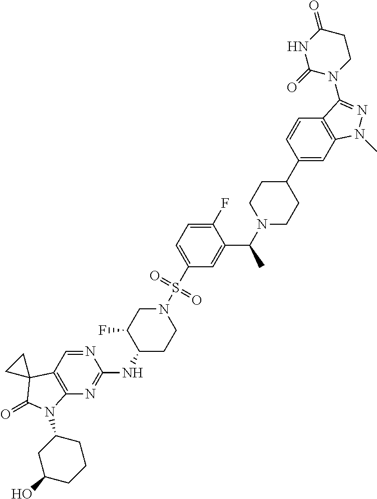 [SMILES: C[C@@H](c1cc(S(=O)(=O)N2CC[C@H](Nc3ncc4c(n3)N([C@@H]3CCC[C@@H](O)C3)C(=O)C43CC3)[C@H](F)C2)ccc1F)N1CCC(c2ccc3c(N4CCC(=O)NC4=O)nn(C)c3c2)CC1] | 201 |
| 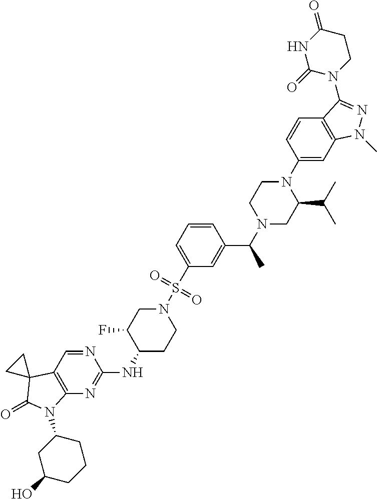 [SMILES: CC(C)[C@H]1CN([C@@H](C)c2cccc(S(=O)(=O)N3CC[C@H](Nc4ncc5c(n4)N([C@@H]4CCC[C@@H](O)C4)C(=O)C54CC4)[C@H](F)C3)c2)CCN1c1ccc2c(N3CCC(=O)NC3=O)nn(C)c2c1] | 202 |
| 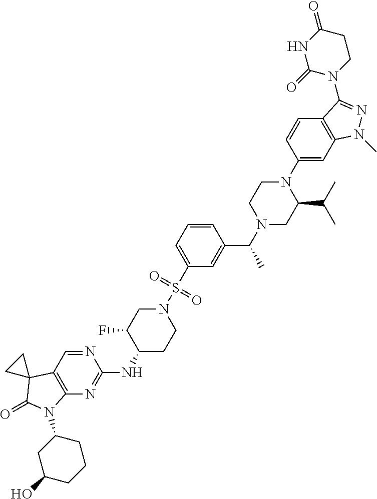 [SMILES: CC(C)[C@H]1CN([C@H](C)c2cccc(S(=O)(=O)N3CC[C@H](Nc4ncc5c(n4)N([C@@H]4CCC[C@@H](O)C4)C(=O)C54CC4)[C@H](F)C3)c2)CCN1c1ccc2c(N3CCC(=O)NC3=O)nn(C)c2c1] | 203 |
| 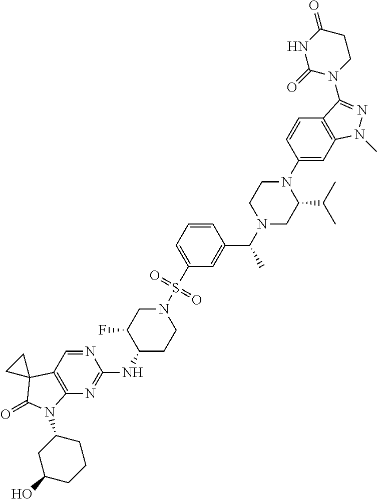 [SMILES: CC(C)[C@@H]1CN([C@H](C)c2cccc(S(=O)(=O)N3CC[C@H](Nc4ncc5c(n4)N([C@@H]4CCC[C@@H](O)C4)C(=O)C54CC4)[C@H](F)C3)c2)CCN1c1ccc2c(N3CCC(=O)NC3=O)nn(C)c2c1] | 204 |
| 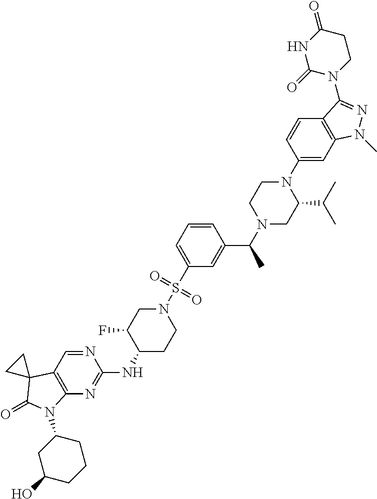 [SMILES: CC(C)[C@@H]1CN([C@@H](C)c2cccc(S(=O)(=O)N3CC[C@H](Nc4ncc5c(n4)N([C@@H]4CCC[C@@H](O)C4)C(=O)C54CC4)[C@H](F)C3)c2)CCN1c1ccc2c(N3CCC(=O)NC3=O)nn(C)c2c1] | 205 |
| 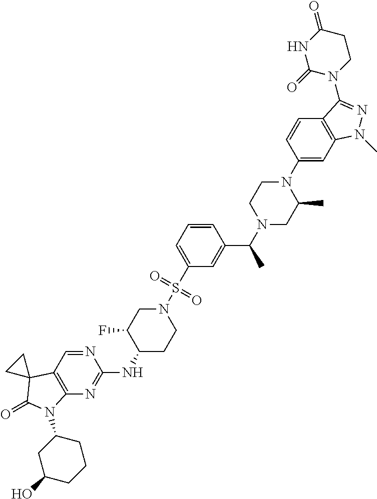 [SMILES: C[C@@H](c1cccc(S(=O)(=O)N2CC[C@H](Nc3ncc4c(n3)N([C@@H]3CCC[C@@H](O)C3)C(=O)C43CC3)[C@H](F)C2)c1)N1CCN(c2ccc3c(N4CCC(=O)NC4=O)nn(C)c3c2)[C@@H](C)C1] | 206 |
| 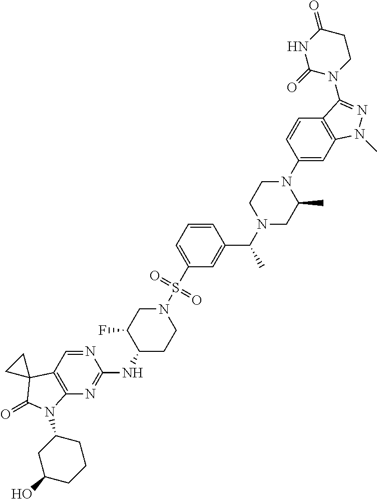 [SMILES: C[C@H](c1cccc(S(=O)(=O)N2CC[C@H](Nc3ncc4c(n3)N([C@@H]3CCC[C@@H](O)C3)C(=O)C43CC3)[C@H](F)C2)c1)N1CCN(c2ccc3c(N4CCC(=O)NC4=O)nn(C)c3c2)[C@@H](C)C1] | 207 |
| 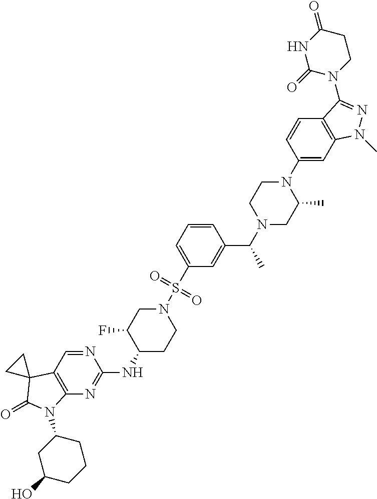 [SMILES: C[C@H](c1cccc(S(=O)(=O)N2CC[C@H](Nc3ncc4c(n3)N([C@@H]3CCC[C@@H](O)C3)C(=O)C43CC3)[C@H](F)C2)c1)N1CCN(c2ccc3c(N4CCC(=O)NC4=O)nn(C)c3c2)[C@H](C)C1] | 208 |
| 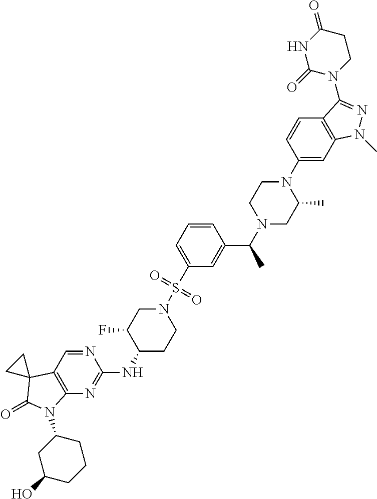 [SMILES: C[C@@H]1CN([C@@H](C)c2cccc(S(=O)(=O)N3CC[C@H](Nc4ncc5c(n4)N([C@@H]4CCC[C@@H](O)C4)C(=O)C54CC4)[C@H](F)C3)c2)CCN1c1ccc2c(N3CCC(=O)NC3=O)nn(C)c2c1] | 209 |
| 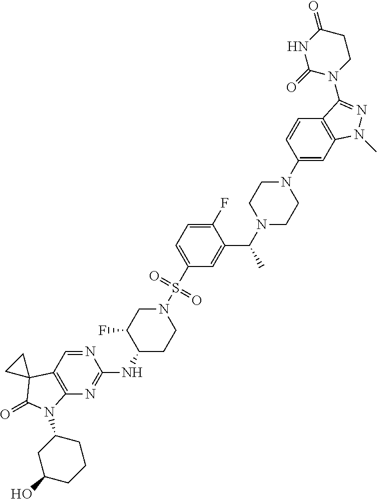 [SMILES: C[C@H](c1cc(S(=O)(=O)N2CC[C@H](Nc3ncc4c(n3)N([C@@H]3CCC[C@@H](O)C3)C(=O)C43CC3)[C@H](F)C2)ccc1F)N1CCN(c2ccc3c(N4CCC(=O)NC4=O)nn(C)c3c2)CC1] | 210 |
| 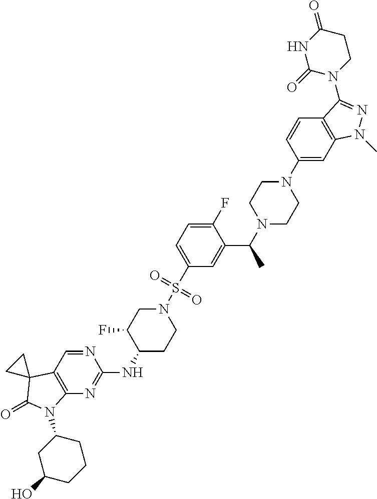 [SMILES: C[C@@H](c1cc(S(=O)(=O)N2CC[C@H](Nc3ncc4c(n3)N([C@@H]3CCC[C@@H](O)C3)C(=O)C43CC3)[C@H](F)C2)ccc1F)N1CCN(c2ccc3c(N4CCC(=O)NC4=O)nn(C)c3c2)CC1] | 211 |

Exemplary compounds

| | CDK2 | CDK2 | CCNE1 | CCNE1 | CDK9 | CDK9 |
|---|---|---|---|---|---|---|
| | HIBIT | HIBIT | HIBIT | HIBIT | HIBIT | HIBIT |
| | Protein | Protein | Protein | Protein | Protein | Protein |
| | Quantification | Quantification | Quantification | Quantification | Quantification | Quantification |
| | DC₅₀ | Maximum | DC₅₀ | Maximum | DC₅₀ | Maximum |
| Nr | (nM) | (%) | (nM) | (%) | (nM) | (%) |
| 1 | 16 | 70.2 | NA | 30.9 | NA | NA |
| 2 | NA | 45.7 | NA | 41.1 | NA | NA |
| 3 | NA | 34.6 | NA | 19.4 | NA | NA |
| 4 | 6.7 | 56.5 | NA | 21.2 | NA | NA |
| 5 | NA | 24.9 | NA | 21.9 | NA | NA |
| 6 | 14.6 | 66.2 | NA | 30 | NA | NA |
| 7 | NA | 37.9 | NA | 21.2 | NA | NA |
| 8 | 8.8 | 59.2 | NA | 24.7 | NA | NA |
| 9 | NA | 48.4 | NA | NA | NA | NA |
| 10 | NA | 38.9 | NA | NA | NA | NA |
| 11 | NA | 49.5 | NA | 23.6 | NA | NA |
| 12 | NA | 34.6 | NA | 16.9 | NA | NA |
| 13 | 17.3 | 79 | 17.6 | 52.8 | NA | NA |
| 14 | 4.56 | 57.4 | NA | 37.7 | NA | NA |
| 15 | | 27 | | 15 | | −3 |
| 16 | | 49 | | 17 | 3.67 | 57 |
| 17 | 17 | 54 | | 26 | | 26 |
| 18 | 12 | 63 | | 20 | | 5 |
| 19 | | 19 | | 26 | | 25 |
| 20 | | 46 | | 29 | 86.3 | 71 |
| 21 | 41 | 54 | | 28 | | 28 |
| 22 | 44 | 57 | | 18 | | 35 |
| 23 | 98 | 64 | | 20 | | 49 |
| 24 | | 41 | | 21 | | −1 |
| 25 | | 29 | | 25 | | 15 |
| 26 | | 42 | | 8 | | 9 |
| 27 | 19 | 57 | | 27 | | 49 |
| 28 | | 42 | | 8 | | 8 |
| 29 | 35 | 76 | | 47 | 95.8 | 57 |
| 30 | 11 | 72 | | 37 | 17.6 | 75 |
| 31 | 17 | 62 | | 14 | | 15 |
| 32 | 86 | 59 | | 22 | | 41 |
| 33 | | 42 | | 9 | | 4 |
| 34 | 6 | 72 | | 44 | 13.6 | 61 |
| 35 | | 34 | | 21 | | 17 |
| 36 | | 46 | 515 | 59 | | 49 |
| 37 | | 40 | | 30 | | 0 |
| 38 | 4 | 71 | | 46 | 23 | 57 |
| 39 | 7 | 54 | | 9 | | 6 |
| 40 | 5 | 69 | | 29 | | 44 |
| 41 | 11 | 65 | | 15 | | 33 |
| 42 | 7 | 70 | | 23 | | 17 |
| 43 | 16 | 60 | | 62 | | 32 |
| 44 | | 39 | | 44 | | 5 |
| 45 | 8 | 59 | | 18 | | 41 |
| 46 | 5 | 74 | | 35 | | 44 |
| 47 | | 44 | | 18 | | 10 |
| 48 | | 35 | | 42 | | 15 |
| 49 | 34 | 62 | | 8 | 6.53 | 66 |
| 50 | | 37 | | 16 | | 17 |
| 51 | 53 | 56 | | 40 | | 41 |
| 52 | 2 | 56 | | 19 | | 29 |
| 53 | 10 | 62 | | 46 | | 36 |
| 54 | | 14 | | | | 4 |
| 55 | | 25 | | 13 | | 8 |
| 56 | | 41 | | 19 | | 15 |
| 57 | | 53 | | 22 | | 15 |
| 58 | | 12 | | 10 | | 9 |
| 59 | | 46 | | 24 | | 17 |
| 60 | | 18 | | 17 | | 28 |
| 61 | 3 | 60 | | 29 | | 23 |
| 62 | | 40 | | 23 | | −6 |
| 63 | | 15 | | 2 | | 14 |
| 64 | | 29 | | 18 | | 17 |
| 65 | | 30 | | 4 | | 9 |
| 66 | | 39 | | 14 | | 8 |
| 67 | | 26 | | 21 | | 16 |
| 68 | | 38 | | 9 | | 6 |
| 69 | | 13 | | 8 | | 8 |
| 70 | | −3 | | 6 | | 15 |
| 71 | | 51 | | 18 | | 15 |
| 72 | | 25 | | 8 | | 42 |
| 73 | 3 | 52 | | 19 | | 41 |
| 74 | | 19 | | 29 | | 8 |
| 75 | | 40 | | 51 | | 29 |
| 76 | | 29 | | 20 | | 9 |
| 77 | | 46 | | 25 | | 40 |
| 78 | | 29 | | 17 | | 23 |
| 79 | | 6 | | 16 | | 21 |
| 80 | | 24 | | 29 | | 8 |
| 81 | 8 | 50 | | 15 | | 14 |
| 82 | | 26 | | 43 | | 11 |
| 83 | | 29 | | 13 | | 16 |
| 84 | 21 | 53 | | 47 | | 30 |
| 85 | | 17 | | 41 | | 14 |
| 86 | 16 | 65 | | 37 | | 34 |
| 87 | | 44 | | 11 | | 13 |
| 88 | 7 | 55 | | 21 | | 24 |
| 89 | | 50 | | 25 | | 35 |
| 90 | 5 | 67 | | 34 | | 45 |
| 91 | 6 | 64 | | 25 | | 37 |
| 92 | | 39 | | 7 | | 26 |
| 93 | | 13 | | 13 | | −3 |
| 94 | 16 | 63 | | 39 | 2 | 51 |
| 95 | 11 | 65 | | 15 | | 7 |
| 96 | | 46 | | 46 | | 26 |
| 97 | | 41 | | 14 | | 46 |
| 98 | | 31 | | | | 15 |
| 99 | | 31 | | 1 | | 11 |
| 100 | 6 | 62 | | 31 | | 34 |
| 101 | | 38 | | 47 | | 23 |
| 102 | | 25 | | 25 | | 20 |
| 103 | | 31 | | 7 | | 0 |
| 104 | | 25 | | 6 | | 11 |
| 105 | | 40 | | 15 | | 36 |
| 106 | | 32 | | 3 | | 12 |
| 107 | | 28 | | 25 | | 22 |
| 108 | | 42 | | 16 | | 4 |
| 109 | 4 | 57 | | 22 | | 47 |
| 110 | | 20 | | 5 | | 14 |
| 111 | | 13 | | 9 | | 25 |
| 112 | | 22 | | 14 | | 28 |
| 113 | | 21 | | 28 | | 6 |
| 114 | | 37 | | 18 | | 1 |
| 115 | | 23 | | | | 7 |
| 116 | 14 | 86 | 23.2 | 75 | 74.9 | 81 |
| 117 | 10 | 78 | 7.43 | 64 | 21.8 | 65 |
| 118 | 7 | 76 | | 44 | | 14 |
| 119 | 3 | 70 | | 22 | | 25 |
| 120 | 23 | 62 | | 23 | | 25 |
| 121 | 47 | 79 | | 44 | | 12 |
| 122 | 7 | 79 | | 25 | | 22 |
| 123 | 92 | 52 | | 47 | | 8 |
| 124 | 6 | 61 | | 48 | 73.6 | 57 |
| 125 | 4 | 54 | 2.35 | 81 | | 13 |
| 126 | 6 | 66 | 29.6 | 71 | 33.4 | 84 |
| 127 | 6 | 69 | | 46 | | 49 |
| 128 | | 32 | | 32 | | 34 |
| 129 | 42 | 51 | 0 | 18 | | 17 |
| 130 | 15.8 | 76 | 0 | 34 | | 13 |
| 131 | 21.9 | 83 | 19.9 | 63 | 69.2 | 56 |
| 132 | | 48 | | 47 | | 7 |
| 133 | 9.23 | 61 | | 26 | | 14 |
| 134 | 4.22 | 74 | | 18 | | 31 |
| 135 | 16.3 | 54 | | 8 | | 8 |
| 136 | 131 | 61 | | 7 | | 14 |
| 137 | 3.25 | 52 | | 38 | | 8 |
| 138 | | 45 | | 14 | | 3 |
| 139 | 3.23 | 65 | | 50 | | 11 |
| 140 | 2.09 | 71 | | 20 | | 8 |
| 141 | | 49 | | 25 | | 27 |
| 142 | 36.1 | 53 | | 26 | | 5 |
| 143 | | 11 | | 1 | | −2 |
| 144 | 1.97 | 75 | | 39 | | 49 |
| 145 | 4.08 | 79 | | 25 | | 14 |
| 146 | 30 | 76 | 36.2 | 82 | | 4 |
| 147 | 11 | 64 | | 13 | | 2 |
| 148 | 6 | 78 | | 44 | | 2 |
| 149 | 13.9 | 69 | | 11 | | 17 |
| 150 | 12.9 | 67 | | 41 | | 45 |
| 151 | 3.31 | 80 | 78.5 | 69 | | 3 |
| 152 | 4.22 | 88 | 23.6 | 53 | 2170 | 52 |
| 153 | 9.42 | 57 | | 28 | | 17 |
| 154 | 7.87 | 64 | | 31 | | 15 |
| 155 | | 19 | | 14 | | 8 |
| 156 | 1.66 | 81 | | 44 | | 9 |
| 157 | | 18 | | 14 | | 12 |
| 158 | 2.64 | 80 | 5.96 | 56 | | 13 |
| 159 | 2.83 | 68 | 5.99 | 54 | | 49 |
| 160 | 25.2 | 75 | | 34 | | 38 |
| 161 | 6.06 | 79 | | 45 | | 17 |
| 162 | 8.03 | 75 | | 42 | | 4 |
| 163 | | 26 | | 38 | 25.3 | 51 |
| 164 | 2.63 | 85 | 6.18 | 54 | | 12 |
| 165 | 10.1 | 78 | | 44 | | 13 |
| 166 | 21.3 | 64 | | 44 | | 18 |
| 167 | 3.77 | 70 | | 32 | | 15 |
| 168 | 2.91 | 71 | | 40 | | 26 |
| 169 | 5.85 | 84 | | 47 | | 10 |
| 170 | 3.63 | 75 | | 46 | | 5 |
| 171 | 4.43 | 55 | | 29 | | 15 |
| 172 | 4.05 | 74 | | 49 | | 17 |
| 174 | 3.58 | 82 | | 47 | | |
| 175 | 4.19 | 78 | | 44 | | 8 |
| 176 | 3.34 | 79 | | 45 | | 20 |
| 177 | 6.51 | 81 | | 41 | | 14 |
| 178 | 1.8 | 75 | | 38 | | 23 |
| 179 | 7.82 | 78 | | 20 | | 15 |
| 180 | 7.18 | 85 | | 48 | | 34 |
| 181 | 6.02 | 77 | | 38 | | 11 |
| 182 | 4.8 | 82 | 8.09 | 52 | | 46 |
| 183 | 2.84 | 67 | | 43 | | 24 |
| 184 | 5.47 | 90 | | 43 | | 22 |
| 185 | 6.77 | 84 | | 36 | | 5 |
| 186 | 2.82 | 62 | | 43 | | 27 |
| 187 | 4.32 | 91 | | 45 | | 11 |
| 188 | 1.92 | 85 | | 45 | | 24 |
| 189 | 2.62 | 88 | | 47 | | |
| 190 | 13.6 | 70 | | 27 | | |
| 191 | 21.9 | 90 | | 48 | | 9 |
| 192 | 4.67 | 92 | | 45 | | 16 |
| 193 | 10.3 | 85 | | 48 | | |
| 194 | 3.86 | 94 | | 42 | | |
| 195 | 5.54 | 92 | | 41 | | 21 |
| 196 | 12 | 93 | | 36 | | 14 |
| 197 | 10.5 | 91 | | 33 | | 8 |
| 198 | 2.48 | 80 | | 40 | | 2 |
| 199 | 3.79 | 88 | 9.5 | 53 | | 15 |
| 200 | 3.81 | 89 | 9.88 | 61 | | 13 |
| 201 | 5.66 | 88 | | 47 | | 23 |
| 202 | 10.9 | 88 | | 37 | | 20 |
| 203 | 10.2 | 85 | | 34 | | 18 |
| 204 | 10.4 | 85 | | 33 | | 15 |
| 205 | 13.1 | 88 | | 38 | | 14 |
| 206 | 10.9 | 91 | | 43 | | 13 |
| 207 | 3.5 | 90 | | 38 | | 23 |
| 208 | 4.62 | 91 | | 45 | | 14 |
| 209 | 12.7 | 91 | | 43 | | 16 |
| 210 | 4.7 | 91 | | 47 | | |
| 211 | 12.1 | 88 | | 40 | | |

### Alternative Embodiments

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

In an embodiment, provided is a pharmaceutical composition comprising a pharmaceutically acceptable carrier and an effective amount of a compound described herein (e.g., a compound of Formula A, Formula I, Formula I-1 or a compound of Table 1), or a pharmaceutically acceptable salt thereof.

The term “pharmaceutically acceptable carrier or adjuvant” refers to a carrier or adjuvant that may be administered to a patient, together with a compound provided herewith, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions provided herewith include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose based substances, polyethylene glycol, sodium carboxymethylcellulose, polyacrylates, waxes, polyethylene polyoxypropylene block polymers, polyethylene glycol and wool fat. Cyclodextrins such as α-, β-, and γ-cyclodextrin, or chemically modified derivatives such as hydroxyalkylcyclodextrins, including 2 and 3 hydroxypropyl-o-cyclodextrins, or other solubilized derivatives may also be advantageously used to enhance delivery of compounds of the formulae described herein.

When employed as pharmaceuticals, the compounds provided herein are typically administered in the form of a pharmaceutical composition. Such compositions can be prepared in a manner well known in the pharmaceutical art and comprise at least one active compound.

In an embodiment, with respect to the pharmaceutical composition, the carrier is a parenteral carrier, oral or topical carrier.

Also provided is a compound described herein (e.g., a compound of Formula A, Formula I, Formula I-1 or a compound of Table 1, or pharmaceutically acceptable salts thereof) (or pharmaceutical composition thereof) for use as a pharmaceutical or a medicament (e.g., a medicament for the treatment of a CDK2 or CCNE (CCNE1 and/or CCNE2)-mediated disease or disorder in a subject in need thereof). In an embodiment, the disease is a CDK2 mediated disease. In an embodiment, the disease is a CCNE (CCNE1 and/or CCNE2)-mediated disease. In an embodiment, the disease is a CDK2 and a CCNE (CCNE1 and/or CCNE2)-mediated disease. In an embodiment, the disease or disorder is a proliferating disease or disorder. In a further embodiment, the disease or disorder is a cancer. In an embodiment, the cancer is selected from ovarian cancer, gastric cancer, uterine cancer (e.g., endometrial cancer), and breast cancer (e.g., triple negative breast cancer (TNBC), hormone-receptor positive (HR+) breast cancer, HER2 positive (HER2+) positive breast cancer).

Also provided is a compound described herein (e.g., a compound of Formula A, Formula I, Formula I-1 or a compound of Table 1, or pharmaceutically acceptable salts thereof) (or pharmaceutical composition thereof) for use in the treatment of a CDK2 or CCNE (CCNE1 and/or CCNE2)-mediated disease or disorder in a subject in need thereof. In an embodiment, the disease is a CDK2 mediated disease. In an embodiment, the disease is a CCNE (CCNE1 and/or CCNE2)-mediated disease. In an embodiment, the disease is a CDK2 and a CCNE (CCNE1 and/or CCNE2)-mediated disease. In an embodiment, the disease or disorder is a proliferating disease or disorder. In a further embodiment, the disease or disorder is a cancer. In an embodiment, the cancer is selected from ovarian cancer, gastric cancer, uterine cancer (e.g., endometrial cancer), and breast cancer (e.g., triple negative breast cancer (TNBC), hormone-receptor positive (HR+) breast cancer, HER2 positive (HER2+) positive breast cancer).

Also provided is a compound described herein (e.g., a compound of Formula A, Formula I, Formula I-1 or a compound of Table 1, or pharmaceutically acceptable salts thereof) (or pharmaceutical composition thereof) for use in the manufacturing of a medicament (e.g., a medicament for the treatment of a CDK2 or CCNE (CCNE1 and/or CCNE2)-mediated disease or disorder in a subject in need thereof). In an embodiment, the disease or disorder is a proliferating disease or disorder. In an embodiment, the disease is a CDK2 mediated disease. In an embodiment, the disease is a CCNE (CCNE1 and/or CCNE2)-mediated disease. In an embodiment, the disease is a CDK2 and a CCNE (CCNE1 and/or CCNE2)-mediated disease. In a further embodiment, the disease or disorder is a cancer. In an embodiment, the cancer is selected from ovarian cancer, gastric cancer, uterine cancer (e.g., endometrial cancer), and breast cancer (e.g., triple negative breast cancer (TNBC), hormone-receptor positive (HR+) breast cancer, HER2 positive (HER2+) positive breast cancer). Generally, the compounds provided herein are administered in an effective amount (e.g., a therapeutically effective amount). The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions provided herewith may be administered orally, parenterally, by inhalation spray, topically, rectally, nasally, buccally, vaginally or via an implanted reservoir, preferably by oral administration or administration by injection. The pharmaceutical compositions provided herewith may contain any conventional nontoxic pharmaceutically acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like. The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The pharmaceutical compositions provided herewith may also be administered in the form of suppositories for rectal administration. These compositions can be prepared by mixing a compound provided herewith with a suitable nonirritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions provided herewith may be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The above-described components for orally administrable, injectable or topically administrable, rectally administrable and nasally administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference. The compounds described herein can also be administered in sustained release forms or from sustained release drug delivery systems. A description of representative sustained release materials can be found in Remington's Pharmaceutical Sciences.

When the compositions provided herewith comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds provided herewith. Alternatively, those agents may be part of a single dosage form, mixed together with the compounds provided herewith in a single composition.

Also provided is the pharmaceutically acceptable acid addition salt of a compound described herein (e.g., compound of Formula A, Formula I, Formula I-1 or a compound of Table 1).

The acid which may be used to prepare the pharmaceutically acceptable salt is that which forms a non-toxic acid addition salt, i.e., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

The compounds described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions provided herewith will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination provided herewith may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long term basis upon any recurrence of disease symptoms.

### Methods of Treatment

Compounds of the present disclosure can inhibit CDK2 and/or CCNE (CCNE1 and/or CCNE2) and therefore are useful for treating diseases wherein the underlying pathology is, wholly or partially, mediated by CDK2 and/or CCNE (CCNE1 and/or CCNE2). In an embodiment, the disease pathology is wholly or partially, mediated by CDK2. In an embodiment, the disease pathology is wholly or partially, mediated by CCNE (CCNE1 and/or CCNE2). In an embodiment, the disease pathology is wholly or partially, mediated by CDK2 and CCNE (CCNE1 and/or CCNE2). Such diseases include cancer and other diseases with proliferation disorder.

In an embodiment, the compounds of Formula A, Formula I and Formula I-1 inhibit both CDK2 and CCNE (CCNE1 and/or CCNE2). In an embodiment the compounds of Formula A, Formula I and Formula I-1 inhibit CDK2 (e.g., selectively inhibit CDK2 over CCNE (CCNE1 and/or CCNE2)). In an embodiment the compounds of Formula A, Formula I and Formula I-1 inhibit CCNE (CCNE1 and/or CCNE2) (e.g., selectively inhibit CCNE (CCNE1 and/or CCNE2) over CDK2).

In an embodiment, the present disclosure provides treatment of an individual or a patient in vivo using a compound of Formula A, Formula I or Formula I-1 or a salt thereof such that growth of cancerous tumors is inhibited. A compound of Formula A, Formula I, Formula I-1 or of any of the formulas as described herein, or a compound as recited in any of the claims and described herein, or a salt thereof, can be used to inhibit the growth of cancerous tumors with aberrations that activate the CDK2 kinase activity. These include, but are not limited to, disease (e.g., cancers) that are characterized by amplification or overexpression of CCNE (CCNE1 and/or CCNE2) such as ovarian cancer, uterine carcinosarcoma and breast cancer and p27 inactivation such as breast cancer and melanomas. Accordingly, in an embodiment of the methods, the patient has been previously determined to have an amplification of the cyclin E (CCNE (CCNE1 and/or CCNE2)) gene and/or an expression level of CCNE (CCNE1 and/or CCNE2) in a biological sample obtained from the human subject that is higher than a control expression level of CCNE (CCNE1 and/or CCNE2). In some embodiments, the cancers are characterized by amplification or overexpression of CCNE1. Accordingly, in an embodiment of the methods, the patient has been previously determined to have an amplification of the cyclin E1 gene and/or an expression level of CCNE1 in a biological sample obtained from the human subject that is higher than a control expression level of CCNE1. Alternatively, a compound of Formula A, Formula I, Formula I-1 or of any of the formulas as described herein, or a compound as recited in any of the claims and described herein, or a salt thereof, can be used in conjunction with other agents or standard cancer treatments.

In an embodiment, the present disclosure provides a method for inhibiting growth of tumor cells in vitro. The method includes contacting the tumor cells in vitro with a compound of Formula A, Formula I, Formula I-1 or of any of the formulas as described herein, or of a compound as recited in any of the claims and described herein, or of a salt thereof.

In an embodiment, the present disclosure provides a method for inhibiting growth of tumor cells with CCNE (CCNE1 and/or CCNE2) amplification and overexpression in an individual or a patient. The method includes administering to the individual or patient in need thereof a therapeutically effective amount of a compound of Formula A, Formula I, Formula I-1 or of any of the formulas as described herein, or of a compound as recited in any of the claims and described herein, or a salt or a stereoisomer thereof.

In an embodiment, provided herein is a method of inhibiting and/or degrading CDK2 and/or CCNE (CCNE1 and/or CCNE2), comprising contacting the CDK2 and/or CCNE (CCNE1 and/or CCNE2) with a compound of Formula A, Formula I, Formula I-1 or any of the formulas as described herein, a compound as recited in any of the claims and described herein, or a salt thereof. In an embodiment, provided herein is a method of inhibiting and/or degrading CDK2, comprising contacting the CDK2 with a compound of Formula A, Formula I, Formula I-1 or any of the formulas as described herein, a compound as recited in any of the claims and described herein, or a salt thereof. In an embodiment, provided herein is a method of inhibiting and/or degrading CCNE (CCNE1 and/or CCNE2), comprising contacting the CCNE (CCNE1 and/or CCNE2) with a compound of Formula A, Formula I, Formula I-1 or any of the formulas as described herein, a compound as recited in any of the claims and described herein, or a salt thereof. In an embodiment, provided herein is a method of inhibiting and/or degrading CDK2 and CCNE (CCNE1 and/or CCNE2), comprising contacting the CDK2 and CCNE (CCNE1 and/or CCNE2) with a compound of Formula A, Formula I, Formula I-1 or any of the formulas as described herein, a compound as recited in any of the claims and described herein, or a salt thereof.

In an embodiment, provided herein is a method of inhibiting and/or degrading CDK2 and/or CCNE (CCNE1 and/or CCNE2) in a patient, comprising administering to the patient a compound of Formula A, Formula I, Formula I-1 or any of the formulas as described herein, a compound as recited in any of the claims and described herein, or a salt thereof.

In an embodiment, provided herein is a method for treating cancer. The method includes administering to a patient (in need thereof), a therapeutically effective amount of a compound of Formula A, Formula I, Formula I-1 or any of the formulas as described herein, a compound as recited in any of the claims and described herein, or a salt thereof.

In an embodiment, the cancer is characterized by amplification or overexpression of CCNE (CCNE1 and/or CCNE2). In an embodiment, the cancer is ovarian cancer or breast cancer, characterized by amplification or overexpression of CCNE (CCNE1 and/or CCNE2).

In an embodiment, provided herein is a method of treating a disease or disorder associated with CDK2 and/or CCNE (CCNE1 and/or CCNE2) in a patient, comprising administering to the patient a therapeutically effective amount of a compound of Formula A, Formula I, Formula I-1 or any of the formulas as described herein, a compound as recited in any of the claims and described herein, or a salt thereof. In an embodiment, provided herein is a method of treating a disease or disorder associated with CDK2 in a patient, comprising administering to the patient a therapeutically effective amount of a compound of Formula A, Formula I, Formula I-1 or any of the formulas as described herein, a compound as recited in any of the claims and described herein, or a salt thereof. In an embodiment, provided herein is a method of treating a disease or disorder associated with CCNE (CCNE1 and/or CCNE2) in a patient, comprising administering to the patient a therapeutically effective amount of a compound of Formula A, Formula I, Formula I-1 or any of the formulas as described herein, a compound as recited in any of the claims and described herein, or a salt thereof. In an embodiment, provided herein is a method of treating a disease or disorder associated with CDK2 and CCNE (CCNE1 and/or CCNE2) in a patient, comprising administering to the patient a therapeutically effective amount of a compound of Formula A, Formula I, Formula I-1 or any of the formulas as described herein, a compound as recited in any of the claims and described herein, or a salt thereof.

In an embodiment, the disease or disorder associated with CDK2 and/or CCNE (CCNE1 and/or CCNE2) is associated with an amplification of the cyclin E1 (CCNE1) gene and/or overexpression of CCNE1. In an embodiment, the disease or disorder associated with CDK2 and/or CCNE (CCNE1 and/or CCNE2) is N-myc amplified neuroblastoma cells (see Molenaar, et al., Proc Natl Acad Sci USA 106(31): 12968-12973) K-Ras mutant lung cancers (see Hu, S., et al., Mol Cancer Ther, 2015. 14(11): 2576-85, and cancers with FBW7 mutation and CCNE (CCNE1 and/or CCNE2) overexpression (see Takada, et al., Cancer Res, 2017.77(18):4881-4893).

In an embodiment, the disease or disorder associated with CDK2 and/or CCNE (CCNE1 and/or CCNE2) is lung squamous cell carcinoma, lung adenocarcinoma, pancreatic adenocarcinoma, breast invasive carcinoma, uterine carcinosarcoma, ovarian serous cystadenocarcinoma, stomach adenocarcinoma, esophageal carcinoma, bladder urothelial carcinoma, mesothelioma, or sarcoma.

In an embodiment, the disease or disorder associated with CDK2 and/or CCNE (CCNE1 and/or CCNE2) is lung adenocarcinoma, breast invasive carcinoma, uterine carcinosarcoma, ovarian serous cystadenocarcinoma, or stomach adenocarcinoma.

In an embodiment, the disease or disorder associated with CDK2 and/or CCNE (CCNE1 and/or CCNE2) is an adenocarcinoma, carcinoma, or cystadenocarcinoma. In an embodiment, the disease or disorder associated with CDK2 and/or CCNE (CCNE1 and/or CCNE2) is uterine cancer, ovarian cancer, stomach cancer, esophageal cancer, lung cancer, bladder cancer, pancreatic cancer, or breast cancer.

In an embodiment, the disease or disorder associated with CDK2 and/or CCNE (CCNE1 and/or CCNE2) is a cancer.

In an embodiment, the cancer is characterized by amplification or overexpression of CCNE (CCNE1 and/or CCNE2). In an embodiment, the cancer is ovarian cancer or breast cancer, characterized by amplification or overexpression of CCNE (CCNE1 and/or CCNE2)

In an embodiment of any of the embodiments described herein, the CCNE is CCNE1. In an embodiment, the CCNE is CCNE2. In an embodiment, the CCNE is CCNE1 and CCNE2.

In and embodiment, the cancer has primary or acquired resistance to CDK4/6 inhibition (e.g., is resistant to treatment with CDK4/6 inhibitors).

In an embodiment, the breast cancer is chemotherapy or radiotherapy resistant breast cancer, endocrine resistant breast cancer, trastuzumab resistant breast cancer, or breast cancer demonstrating primary or acquired resistance to CDK4/6 inhibition. In an embodiment, the breast cancer is advanced or metastatic breast cancer.

Additionally, the disclosure includes refractory or recurrent malignancies whose growth may be inhibited using the compounds of the disclosure.

In an embodiment, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

### Selected Embodiments

Embodiment 1. A compound of Formula A:

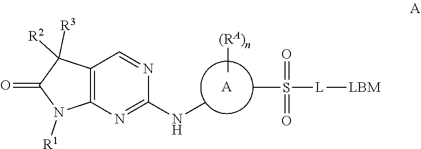

or a pharmaceutically acceptable salt thereof, wherein:
   Ring A is phenyl, 3-14 membered cycloalkyl or 4-14 membered heterocyclyl containing 1-3 heteroatoms independently selected from N, O and S;
   R¹ is selected from —C₁₋₆ alkyl, —C₂₋₆ alkenyl, —C₂₋₆ alkynyl, —C₁₋₆ haloalkyl, —C₁₋₆ heteroalkyl, —C₃₋₁₄ cycloalkyl, 6-14 membered aryl, 4-14 membered heterocyclyl, 5-14 membered heteroaryl, —C₁₋₄ alkyl-C₃₋₁₄ cycloalkyl, —C₁₋₄ alkyl-(6-14 membered aryl), —C₁₋₄ alkyl-(4-14 membered heterocyclyl), and —C₁₋₄ alkyl (5-14 membered heteroaryl), wherein each heterocyclyl and heteroaryl contains 1-3 heteroatoms independently selected from N, O and S, and wherein each alkyl, alkenyl, alkynyl, cycloalkyl, aryl, heterocyclyl, and heteroaryl is substituted with 0, 1, 2, 3 or 4 instances of R⁶;
   each R² and R³ is independently selected from —C₁₋₆ alkyl, —C₁₋₆ haloalkyl, —C₁₋₆ heteroalkyl, —C₂₋₆ alkenyl, —C₂₋₆ alkynyl, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, and wherein each alkyl, alkenyl, alkynyl, cycloalkyl, aryl, heterocyclyl, and heteroaryl is substituted with 0, 1, 2, 3 or 4 instances of R^{B};
   or R² and R³, together with the carbon atom to which they are attached, form Ring B, wherein Ring B is a 3-7 membered cycloalkyl ring or a 4-7 membered heterocyclyl ring containing 1 or 3 heteroatoms independently selected from N, O and S, wherein Ring B is substituted with 0, 1, 2, 3, or 4 instances of R^{B};
   L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(═O)—, —C(═O)O—, —C(═O)—, —S(═O)—, —S(═O)₂—, —NRS(═O)₂—, —S(═O)₂NR—, —NRC(═O)—, —C(═O)NR—, —OC(═O)NR— or —NRC(═O)O—, wherein:
   each -Cy- is independently a bivalent ring selected from phenylene, an 8-10 membered bicyclic arylene, a 4-7 membered monocyclic carbocyclylene, a 5-11 membered spiro carbocyclylene, a 4-10 membered bicyclic carbocyclylene, a 5-10 membered bridged carbocyclylene, a 4-7 membered monocyclic heterocyclylene having 1-2 heteroatoms independently selected from nitrogen, oxygen, and sulfur, a 5-11 membered spiro heterocyclylene having 1-2 heteroatoms independently selected from nitrogen, oxygen, and sulfur, an 4-10 membered bicyclic heterocyclylene having 1-2 heteroatoms independently selected from nitrogen, oxygen, and sulfur, a 5-10 membered bridged bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur, wherein each phenylene, arylene, carbocyclylene, heterocyclylene and heteroarylene is substituted with 0, 1, 2, 3, or 4 instances of R^{C};
LBM is selected from

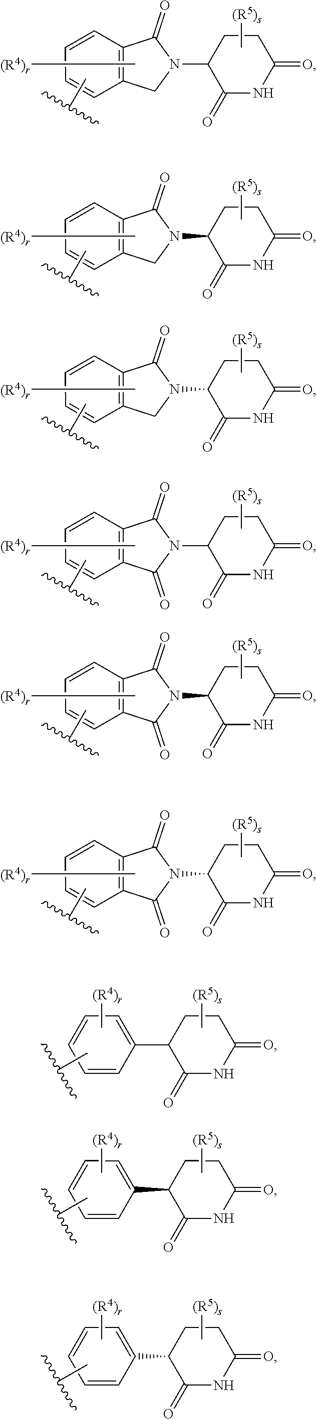

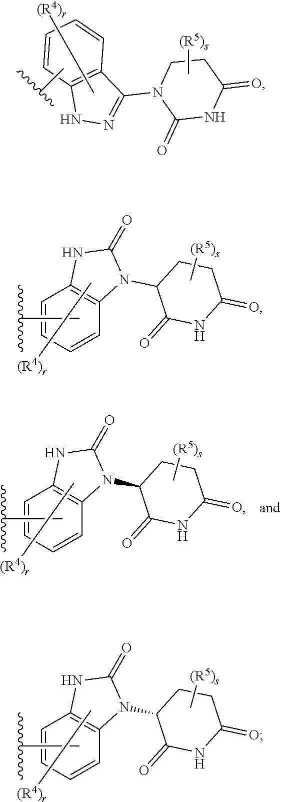

each instance of R^{A}, R^{B}, R^{C}, R⁴, R⁵ and R⁶ is independently selected from oxo, deuterium, halogen, —C₁₋₆ alkyl, —C₁₋₆ heteroalkyl, —C₁₋₆ haloalkyl, —C₂₋₆ alkenyl, —C₂₋₆ alkynyl, —CN, —NO₂, —OR, —SR, —NR₂, —S(O)₂R, —S(O)₂NR₂, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S;
each instance of R is independently hydrogen, —C₁₋₆ alkyl, —C₁₋₆ haloalkyl, —C₁₋₆ heteroalkyl, —C₂₋₆ alkenyl, —C₂₋₆ alkynyl, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, or two R groups attached to the same nitrogen are optionally taken together with nitrogen to which they are attached to form an optionally substituted 4-7 heterocyclyl having 0, 1 or 2 additional heteroatoms independently selected from N, O and S;
n is 0, 1, 2, 3, or 4;
r is 0, 1, 2, 3, or 4; and
s is 0, 1, 2, 3, or 4.

Embodiment 2. The compound of embodiment 1, or a pharmaceutically acceptable salt thereof, wherein the compound is of Formula B:

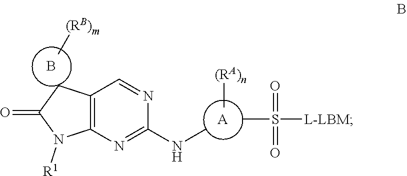

wherein Ring B is a 3-7 membered cycloalkyl ring or a 4-7 membered heterocyclyl ring containing 1 or 3 heteroatoms independently selected from N, O and S.

Embodiment 3. The compound of embodiment 1 or 2, or a pharmaceutically acceptable salt thereof, wherein Ring B is a 3-7 membered cycloalkyl ring.

Embodiment 4. The compound of embodiment 1 or 2, or a pharmaceutically acceptable salt thereof, wherein Ring B is selected from cyclopropyl, cyclobutyl, cyclopentyl and cyclohexyl.

Embodiment 5. The compound of embodiment 1 or 2, or a pharmaceutically acceptable salt thereof, wherein Ring B is selected from cyclopropyl and cyclobutyl.

Embodiment 6. The compound of embodiment 1 or 2, or a pharmaceutically acceptable salt thereof, wherein Ring B is cyclobutyl.

Embodiment 7. The compound of embodiment 1 or 2, or a pharmaceutically acceptable salt thereof, wherein Ring B is cyclopropyl.

Embodiment 8. The compound of any one of embodiments 1-7, or a pharmaceutically acceptable salt thereof, wherein m is 0 and Ring B is unsubstituted.

Embodiment 9. The compound of any one of embodiments 1-8, or a pharmaceutically acceptable salt thereof, wherein the compound is of Formula C:

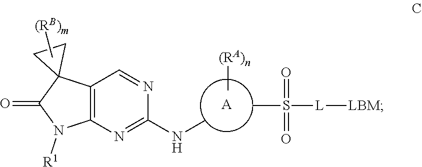

wherein m is 0, 1, 2, 3 or 4.

Embodiment 10. The compound of embodiment 9, or a pharmaceutically acceptable salt thereof, wherein the compound is of Formula D:

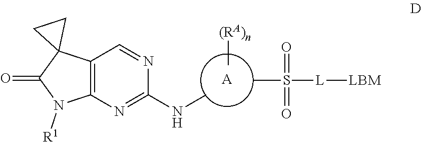

Embodiment 11. The compound of any one of embodiments 1-10, or a pharmaceutically acceptable salt thereof, wherein Ring A is a monocyclic 3-7 membered cycloalkyl.

Embodiment 12. The compound of any one of embodiments 1-10, or a pharmaceutically acceptable salt thereof, wherein Ring A is a monocyclic 4-7 membered heterocyclyl.

Embodiment 13. The compound of any one of embodiments 1-10, or a pharmaceutically acceptable salt thereof, wherein Ring A is selected from azetidine, pyrrolidine, piperidine, and azepane.

Embodiment 14. The compound of any one of embodiments 1-10, or a pharmaceutically acceptable salt thereof, wherein Ring A is piperidine.

Embodiment 15. The compound of any one of embodiments 1-10, or a pharmaceutically acceptable salt thereof, wherein Ring A is piperidin-4-yl.

Embodiment 16. The compound of embodiment 1, or a pharmaceutically acceptable salt thereof, wherein the compound is of Formula E:

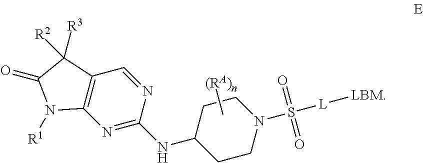

Embodiment 17. The compound of any one of embodiments 1-8, or a pharmaceutically acceptable salt thereof, wherein the compound is of Formula F:

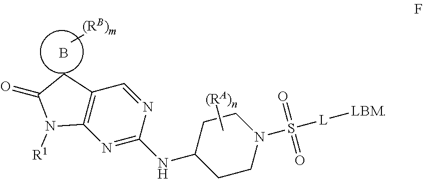

Embodiment 18. A compound of Formula I or Formula I-1:

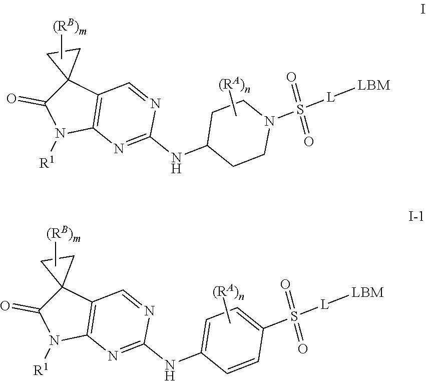

or a pharmaceutically acceptable salt thereof, wherein:
   R¹ is selected from —C₁₋₆ alkyl, —C₂₋₆ alkenyl, —C₂₋₆ alkynyl, —C₁₋₆ haloalkyl, —C₁₋₆ heteroalkyl, —C₃₋₁₄ cycloalkyl, 6-14 membered aryl, 4-14 membered heterocyclyl, 5-14 membered heteroaryl, —C₁₋₄ alkyl-C₃₋₁₄ cycloalkyl, —C₁₋₄ alkyl-(6-14 membered aryl), —C₁₋₄ alkyl-(4-14 membered heterocyclyl), and —C₁₋₄ alkyl (5-14 membered heteroaryl), wherein each heterocyclyl and heteroaryl contains 1-3 heteroatoms independently selected from N, O and S, and wherein each alkyl, alkenyl, alkynyl, cycloalkyl, aryl, heterocyclyl, and heteroaryl is substituted with 0, 1, 2, 3 or 4 instances of R⁶;
   L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(═O)—, —C(═O)O—, —C(═O)—, —S(═O)—, —S(═O)₂—, —NRS(═O)₂—, —S(═O)₂NR—, —NRC(═O)—, —C(═O)NR—, —OC(═O)NR— or —NRC(═O)O—, wherein:
   each -Cy- is independently a bivalent ring selected from phenylene, an 8-10 membered bicyclic arylene, a 4-7 membered monocyclic carbocyclylene, a 5-11 membered spiro carbocyclylene, a 4-10 membered bicyclic carbocyclylene, a 5-10 membered bridged carbocyclylene, a 4-7 membered monocyclic heterocyclylene having 1-2 heteroatoms independently selected from nitrogen, oxygen, and sulfur, a 5-11 membered spiro heterocyclylene having 1-2 heteroatoms independently selected from nitrogen, oxygen, and sulfur, an 4-10 membered bicyclic heterocyclylene having 1-2 heteroatoms independently selected from nitrogen, oxygen, and sulfur, a 5-10 membered bridged bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur, wherein each phenylene, arylene, carbocyclylene, heterocyclylene and heteroarylene is substituted with 0, 1, 2, 3, or 4 instances of Re;
LBM is selected from

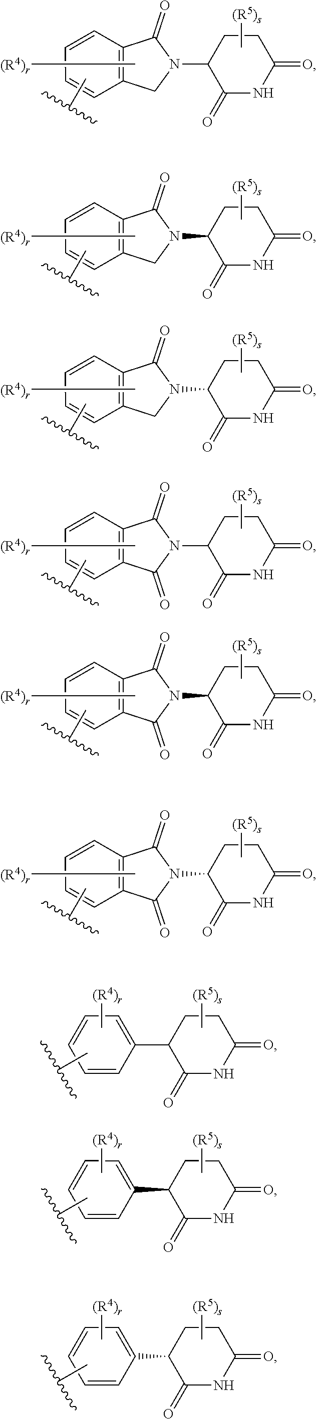

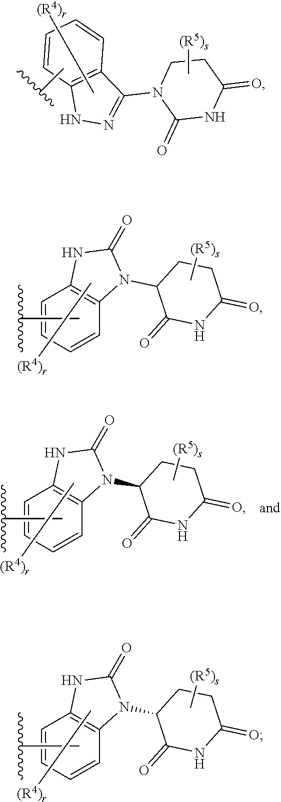

each instance of R^{A}, R^{B}, R^{C}, R⁴, R⁵ and R⁶ is independently selected from oxo, deuterium, halogen, —C₁₋₆ alkyl, —C₁₋₆ heteroalkyl, —C₁₋₆ haloalkyl, —C₂₋₆ alkenyl, —C₂₋₆ alkynyl, —CN, —NO₂, —OR, —SR, —NR₂, —S(O)₂R, —S(O)₂NR₂, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S;
each instance of R is independently hydrogen, —C₁₋₆ alkyl, —C₁₋₆ haloalkyl, —C₁₋₆ heteroalkyl, —C₂₋₆ alkenyl, —C₂₋₆ alkynyl, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, or two R groups attached to the same nitrogen are optionally taken together with nitrogen to which they are attached to form an optionally substituted 4-7 heterocyclyl having 0, 1 or 2 additional heteroatoms independently selected from N, O and S;
m is 0, 1, 2, 3, or 4;
n is 0, 1, 2, 3, or 4;
r is 0, 1, 2, 3, or 4; and
s is 0, 1, 2, 3, or 4.

Embodiment 19. The compound of embodiment 18, or a pharmaceutically acceptable salt thereof, wherein the compound is of formula I.

Embodiment 20. The compound of embodiment 18, or a pharmaceutically acceptable salt thereof, wherein the compound is of formula I-1.

Embodiment 21. The compound of embodiment 18, or a pharmaceutically acceptable salt thereof, wherein the compound is of Formula I-A:

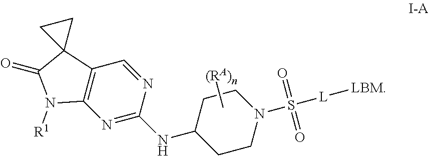

Embodiment 22. The compound of embodiment 18, or a pharmaceutically acceptable salt thereof, wherein the compound is of Formula I-B:

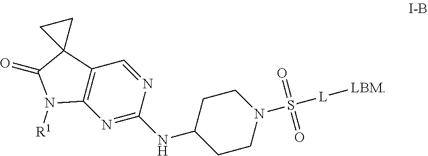

Embodiment 23. The compound of embodiment 18, or a pharmaceutically acceptable salt thereof, wherein the compound is of Formula I-C:

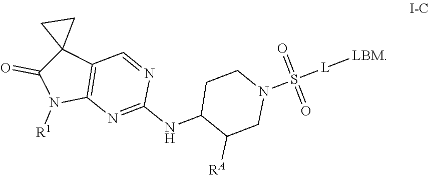

Embodiment 24. The compound of embodiment 18, or a pharmaceutically acceptable salt thereof, wherein the compound is of Formula I-1-A:

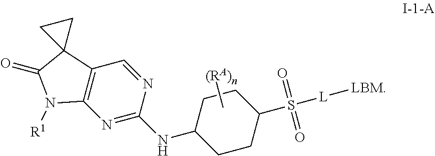

Embodiment 25. The compound of embodiment 18, or a pharmaceutically acceptable salt thereof, wherein the compound is of Formula I-1-B:

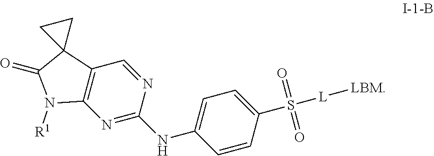

Embodiment 26. The compound of any one of embodiments 1-25, or a pharmaceutically acceptable salt thereof, wherein R¹ is selected from —C₃₋₇ cycloalkyl, phenyl and —C₁₋₄ alkyl-C₃₋₁₄ cycloalkyl, each of which is substituted with 0, 1, 2, 3 or 4 instances of R⁶.

Embodiment 27. The compound of any one of embodiments 1-25, or a pharmaceutically acceptable salt thereof, wherein R¹ is selected from —C₁₋₆ haloalkyl, —C₃₋₇ cycloalkyl, phenyl, 4-7 membered heterocyclyl, and 5-6 membered heteroaryl, each of which is substituted with 0, 1, 2, 3 or 4 instances of R⁶.

Embodiment 28. The compound of any one of embodiments 1-25, or a pharmaceutically acceptable salt thereof, wherein R¹ is selected from C₁₋₆ haloalkyl, C₃₋₇ cycloalkyl, and phenyl, each of which is substituted with 0, 1, 2, 3 or 4 instances of R⁶.

Embodiment 29. The compound of any one of embodiments 1-25, or a pharmaceutically acceptable salt thereof, wherein R¹ is C₃₋₇ cycloalkyl substituted with 0, 1, 2, 3 or 4 instances of R⁶.

Embodiment 30. The compound of any one of embodiments 1-22, or a pharmaceutically acceptable salt thereof, wherein R¹ is cyclopropyl, cyclobutyl, cyclopentyl, cyclohexyl or cycloheptyl substituted with 0, 1, 2, 3 or 4 instances of R⁶.

Embodiment 31. The compound of any one of embodiments 1-25, or a pharmaceutically acceptable salt thereof, wherein R¹ is cyclopentyl, cyclohexyl, phenyl or —CH(CH₃)-cyclopropyl, each substituted with 0, 1, 2, 3 or 4 instances of R⁶.

Embodiment 32. The compound of any one of embodiments 1-25, or a pharmaceutically acceptable salt thereof, wherein R¹ is cyclopentyl, cyclohexyl, phenyl or —CH(CH₃)-cyclopropyl, each substituted with 0, 1 or 2 instances of R⁶.

Embodiment 33. The compound of any one of embodiments 1-25, or a pharmaceutically acceptable salt thereof, wherein R¹ is cyclopentyl or cyclohexyl, each substituted with 0, 1, 2, 3 or 4 instances of R⁶.

Embodiment 34. The compound of any one of embodiments 1-25, or a pharmaceutically acceptable salt thereof, wherein R¹ is cyclopentyl substituted with 0, 1, 2, 3 or 4 instances of R⁶.

Embodiment 35. The compound of embodiment 1, or a pharmaceutically acceptable salt thereof, wherein the compound is of Formula G:

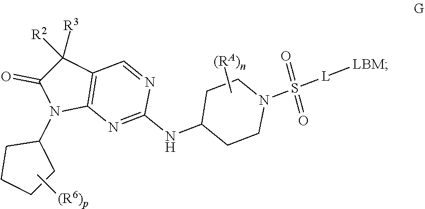

wherein p is 0, 1, 2, 3 or 4.

Embodiment 36. The compound of any one of embodiments 1-Error! Reference source not found., or a pharmaceutically acceptable salt thereof, wherein the compound is of Formula H:

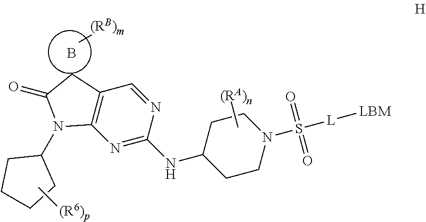

wherein p is 0, 1, 2, 3 or 4.

Embodiment 37. The compound of embodiment 18 or 19, or a pharmaceutically acceptable salt thereof, wherein the compound is of Formula II:

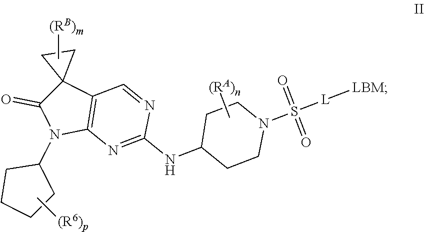

wherein p is 0, 1, 2, 3 or 4.

Embodiment 38. The compound of embodiment 18 or 19, or a pharmaceutically acceptable salt thereof, wherein the compound is of Formula II-A:

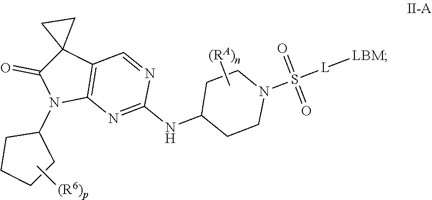

wherein p is 0, 1, 2, 3 or 4.

Embodiment 39. The compound of embodiment 18 or 19, or a pharmaceutically acceptable salt thereof, wherein the compound is of Formula II-B:

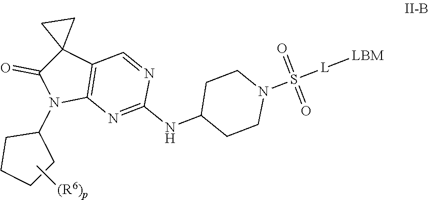

wherein p is 0, 1, 2, 3 or 4.

Embodiment 40. The compound of embodiment 18 or 19, or a pharmaceutically acceptable salt thereof, wherein the compound is of Formula II-C:

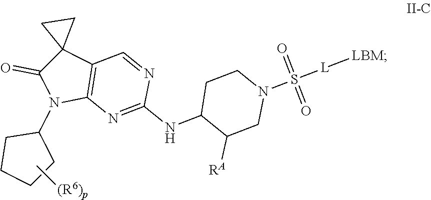

wherein p is 0, 1, 2, 3 or 4.

Embodiment 41. The compound of embodiment 18 or 20, or a pharmaceutically acceptable salt thereof, wherein the compound is of Formula II-1:

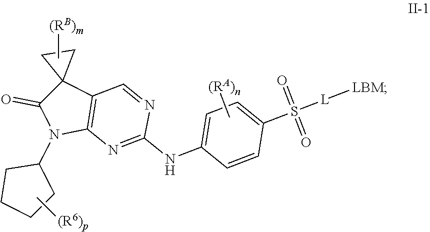

wherein p is 0, 1, 2, 3 or 4.

Embodiment 42. The compound of embodiment 18 or 20, or a pharmaceutically acceptable salt thereof, wherein the compound is of Formula II-1-A:

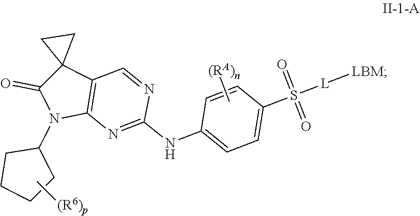

wherein p is 0, 1, 2, 3 or 4.

Embodiment 43. The compound of embodiment 18 or 20, or a pharmaceutically acceptable salt thereof, wherein the compound is of Formula II-1-B:

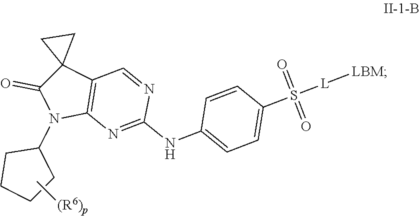

wherein p is 0, 1, 2, 3 or 4.

Embodiment 44. The compound of any one of embodiments 1-25, or a pharmaceutically acceptable salt thereof, wherein R¹ is cyclohexyl substituted with 0, 1, 2, 3 or 4 instances of R⁶.

Embodiment 45. The compound of embodiment 1, or a pharmaceutically acceptable salt thereof, wherein the compound is of Formula J:

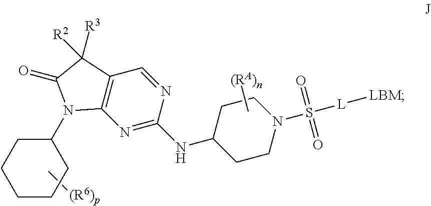

wherein p is 0, 1, 2, 3 or 4.

Embodiment 46. The compound of any one of embodiments 1-17, or a pharmaceutically acceptable salt thereof, wherein the compound is of Formula K:

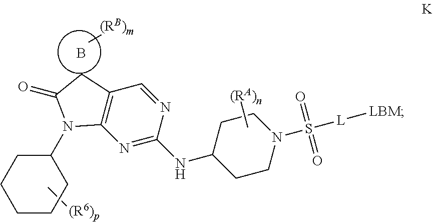

wherein p is 0, 1, 2, 3 or 4.

Embodiment 47. The compound of embodiment 18 or 19, or a pharmaceutically acceptable salt thereof, wherein the compound is of Formula IV:

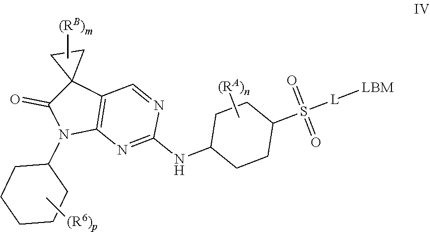

wherein p is 0, 1, 2, 3 or 4.

Embodiment 48. The compound of embodiment 18 or 19, or a pharmaceutically acceptable salt thereof, wherein the compound is of Formula IV-A:

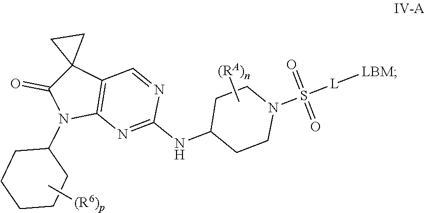

wherein p is 0, 1, 2, 3 or 4.

Embodiment 49. The compound of embodiment 18 or 19, or a pharmaceutically acceptable salt thereof, wherein the compound is of Formula IV-B:

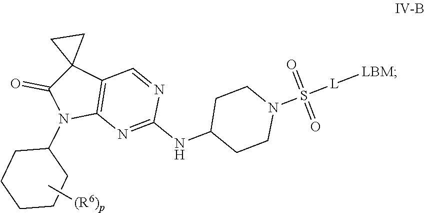

wherein p is 0, 1, 2, 3 or 4.

Embodiment 50. The compound of embodiment 18 or 19, or a pharmaceutically acceptable salt thereof, wherein the compound is of Formula IV-C:

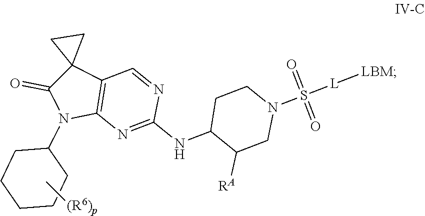

wherein p is 0, 1, 2, 3 or 4.

Embodiment 51. The compound of embodiment 18 or 20, or a pharmaceutically acceptable salt thereof, wherein the compound is of Formula IV-1:

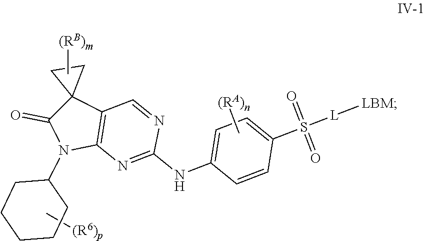

wherein p is 0, 1, 2, 3 or 4.

Embodiment 52. The compound of embodiment 18 or 20, or a pharmaceutically acceptable salt thereof, wherein the compound is of Formula IV-1-A:

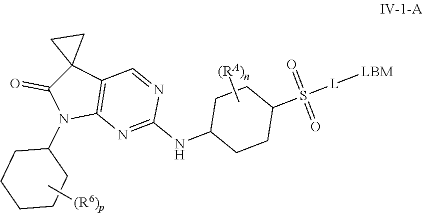

wherein p is 0, 1, 2, 3 or 4.

Embodiment 53. The compound of embodiment 18 or 20, or a pharmaceutically acceptable salt thereof, wherein the compound is of Formula IV-1-B:

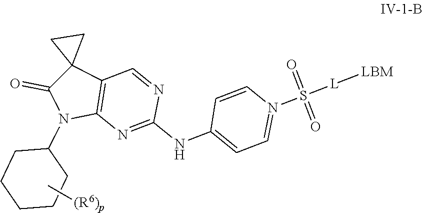

wherein p is 0, 1, 2, 3 or 4.

Embodiment 54. The compound of any one of embodiments 1-53, or a pharmaceutically acceptable salt thereof, wherein R⁶ is selected from halo, —OH and —C₁₋₆ alkyl.

Embodiment 55. The compound of any one of embodiments 1-53, or a pharmaceutically acceptable salt thereof, wherein R⁶ is selected from —F, —Cl, —OH, -Me, -Et-, —ⁱPr.

Embodiment 56. The compound of any one of embodiments 1-53, or a pharmaceutically acceptable salt thereof, wherein R⁶ is selected from —OH, —F and -Me.

Embodiment 57. The compound of any one of embodiments 1-53, or a pharmaceutically acceptable salt thereof, wherein R⁶ is selected from —F and -Me.

Embodiment 58. The compound of any one of embodiments 1-53, or a pharmaceutically acceptable salt thereof, wherein R⁶ is selected from —OH and -Me.

Embodiment 59. The compound of any one of embodiments 1-53, or a pharmaceutically acceptable salt thereof, wherein R⁶ is —OH.

Embodiment 60. The compound of any one of embodiments 1-53, or a pharmaceutically acceptable salt thereof, wherein R⁶ is -Me.

Embodiment 61. The compound of any one of embodiments 1-25, or a pharmaceutically acceptable salt thereof, wherein R¹ is selected from 1,1-difluorobutan-2-yl, cyclopentyl, 2-methylcyclopentyl, 3-hydroxycyclohexyl, 2-hydroxy-2-methylcyclopentyl, 2-methylphenyl, 2-chloro-5-fluorophenyl 1,5-dimethyl-1H-pyrazol-4-yl, 7-chloro-1,2,3,4-tetrahydroisoquinolin-6-yl, and 1-cyclopropylethyl.

Embodiment 62. The compound of any one of embodiments 1-25, or a pharmaceutically acceptable salt thereof, wherein R¹ is selected from cyclopentyl, 2-methylcyclopentyl, 3-hydroxycyclohexyl, 2-methylphenyl, and 1,1-difluorobutan-2-yl.

Embodiment 63. The compound of any one of embodiments 1-25, or a pharmaceutically acceptable salt thereof, wherein R¹ is selected from cyclopentyl, 2-methylcyclopentyl, 2-methylphenyl, and 1,1-difluorobutan-2-yl.

Embodiment 64. The compound of any one of embodiments 1-25, or a pharmaceutically acceptable salt thereof, wherein R¹ is selected from 2-methylcyclopentyl and 3-hydroxycyclohexyl.

Embodiment 65. The compound of any one of embodiments 1-25, or a pharmaceutically acceptable salt thereof, wherein R¹ is 3-hydroxycyclohexyl.

Embodiment 66. The compound of any one of embodiments 1-25, or a pharmaceutically acceptable salt thereof, wherein R¹ is 2-methylcyclopentyl.

Embodiment 67. The compound of any one of embodiments 1-25, or a pharmaceutically acceptable salt thereof, wherein R¹ is selected from:

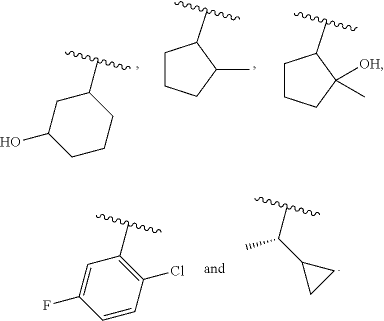

Embodiment 68. The compound of any one of embodiments 1-25, or a pharmaceutically acceptable salt thereof, wherein R¹ is selected from:

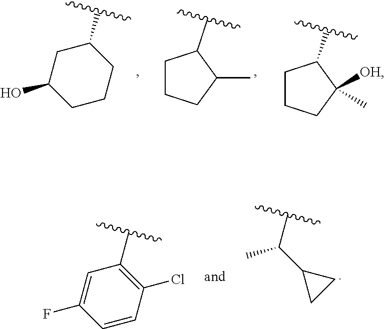

Embodiment 69. The compound of any one of embodiments 1-25, or a pharmaceutically acceptable salt thereof, wherein R¹ is selected from:

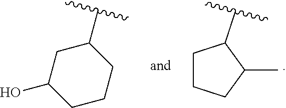

Embodiment 70. The compound of any one of embodiments 1-25, or a pharmaceutically acceptable salt thereof, wherein R¹ is selected from:

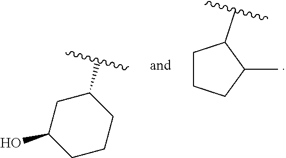

Embodiment 71. The compound of embodiment 1, or a pharmaceutically acceptable salt thereof, wherein the compound is of Formula L:

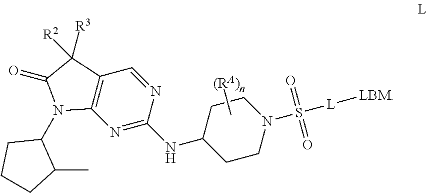

Embodiment 72. The compound of any one of embodiments 1-17, or a pharmaceutically acceptable salt thereof, wherein the compound is of Formula M:

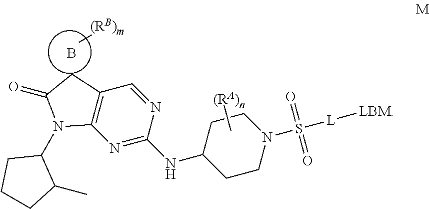

Embodiment 73. The compound of embodiment 1, or a pharmaceutically acceptable salt thereof, wherein the compound is of Formula N:

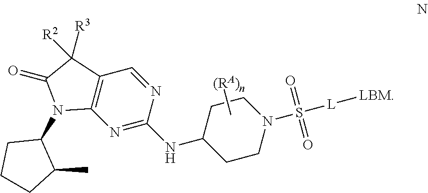

Embodiment 74. The compound of any one of embodiments 1-17, or a pharmaceutically acceptable salt thereof, wherein the compound is of Formula O:

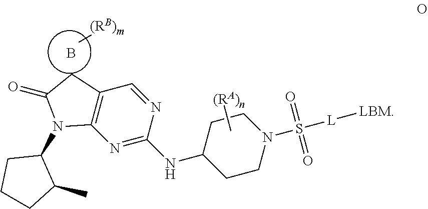

Embodiment 75. The compound of embodiment 18 or 19 or a pharmaceutically acceptable salt thereof, wherein the compound is of Formula III:

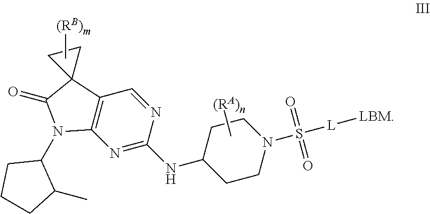

Embodiment 76. The compound of embodiment 18 or 19, or a pharmaceutically acceptable salt thereof, wherein the compound is of Formula III-A:

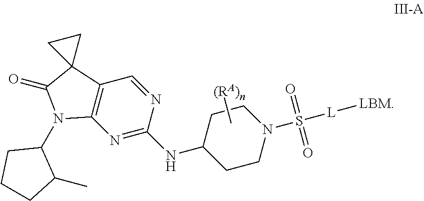

Embodiment 77. The compound of embodiment 18 or 19, or a pharmaceutically acceptable salt thereof, wherein the compound is of Formula III-B:

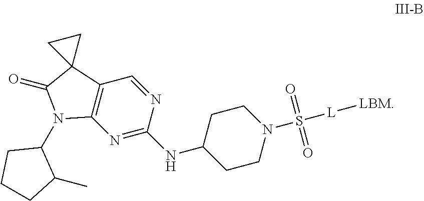

Embodiment 78. The compound of embodiment 18 or 19, or a pharmaceutically acceptable salt thereof, wherein the compound is of Formula III-C:

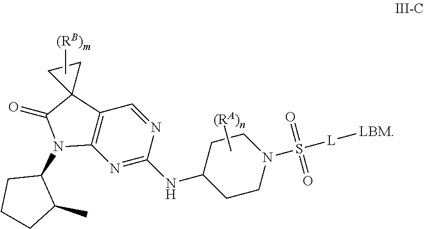

Embodiment 79. The compound of embodiment 18 or 19, or a pharmaceutically acceptable salt thereof, wherein the compound is of Formula III-D:

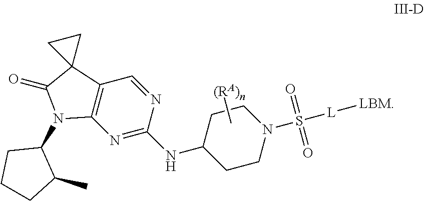

Embodiment 80. The compound of embodiment 18 or 19, or a pharmaceutically acceptable salt thereof, wherein the compound is of Formula III-E:

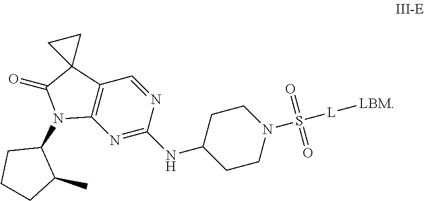

Embodiment 81. The compound of embodiment 18 or 19, or a pharmaceutically acceptable salt thereof, wherein the compound is of Formula III-F:

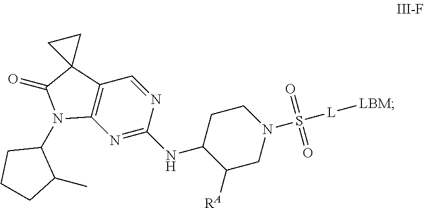

Embodiment 82. The compound of embodiment 18 or 19, or a pharmaceutically acceptable salt thereof, wherein the compound is of Formula III-G:

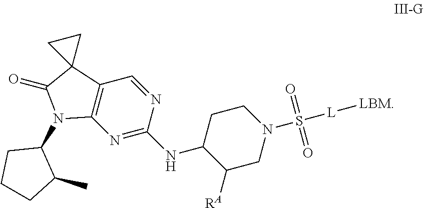

Embodiment 83. The compound of embodiment 18 or 20, or a pharmaceutically acceptable salt thereof, wherein the compound is of Formula III-1:

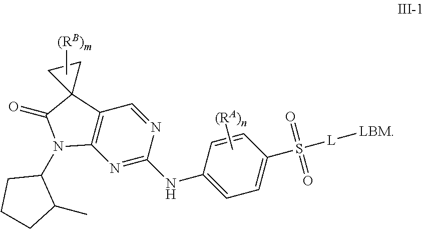

Embodiment 84. The compound of embodiment 18 or 20, or a pharmaceutically acceptable salt thereof, wherein the compound is of Formula III-1-A:

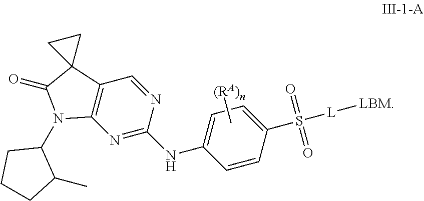

Embodiment 85. The compound of embodiment 18 or 20, or a pharmaceutically acceptable salt thereof, wherein the compound is of Formula III-1-B:

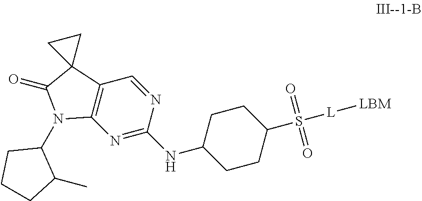

Embodiment 86. The compound of embodiment 18 or 20, or a pharmaceutically acceptable salt thereof, wherein the compound is of Formula III-1-C:

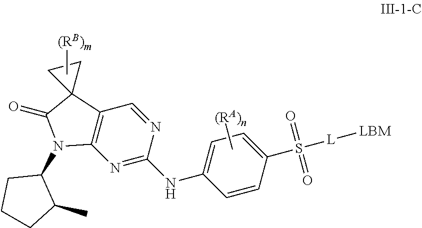

Embodiment 87. The compound of embodiment 18 or 20, or a pharmaceutically acceptable salt thereof, wherein the compound is of Formula III-1-D:

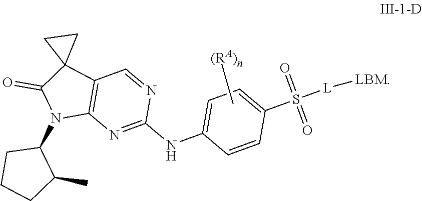

Embodiment 88. The compound of embodiment 18 or 20, or a pharmaceutically acceptable salt thereof, wherein the compound is of Formula III-1-E:

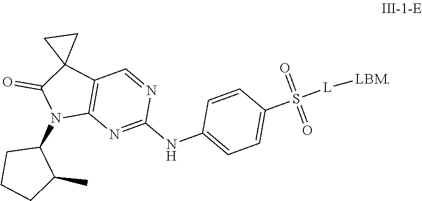

Embodiment 89. The compound of embodiment 18 or 19 or a pharmaceutically acceptable salt thereof, wherein the compound is of Formula V:

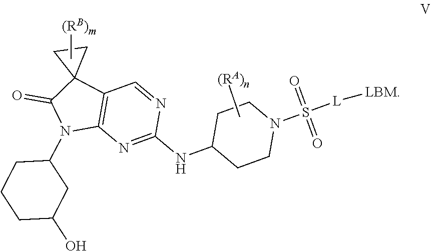

Embodiment 90. The compound of embodiment 18 or 19, or a pharmaceutically acceptable salt thereof, wherein the compound is of Formula V-A:

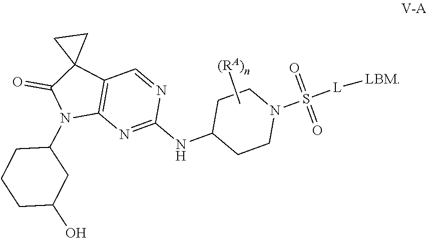

Embodiment 91. The compound of embodiment 18 or 19, or a pharmaceutically acceptable salt thereof, wherein the compound is of Formula V-B:

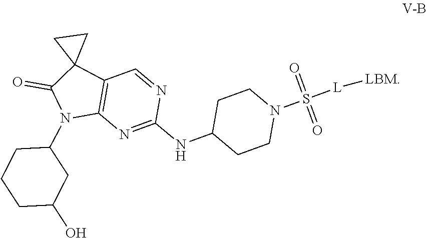

Embodiment 92. The compound of embodiment 18 or 19, or a pharmaceutically acceptable salt thereof, wherein the compound is of Formula V-C:

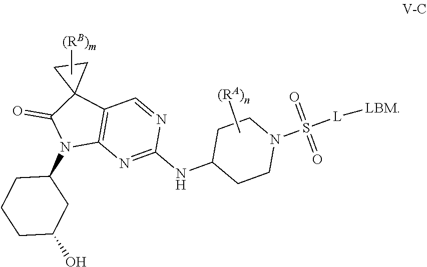

Embodiment 93. The compound of embodiment 18 or 19, or a pharmaceutically acceptable salt thereof, wherein the compound is of Formula V-D:

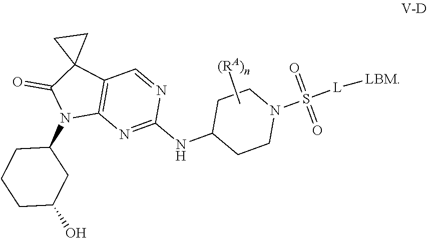

Embodiment 94. The compound of embodiment 18 or 19, or a pharmaceutically acceptable salt thereof, wherein the compound is of Formula V-E:

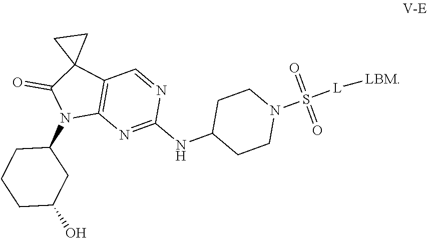

Embodiment 95. The compound of embodiment 18 or 19, or a pharmaceutically acceptable salt thereof, wherein the compound is of Formula V-F:

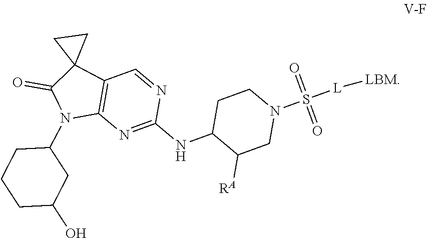

Embodiment 96. The compound of embodiment 18 or 19, or a pharmaceutically acceptable salt thereof, wherein the compound is of Formula V-G:

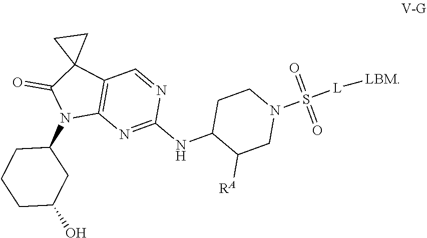

Embodiment 97. The compound of embodiment 18 or 20 or a pharmaceutically acceptable salt thereof, wherein the compound is of Formula V-1:

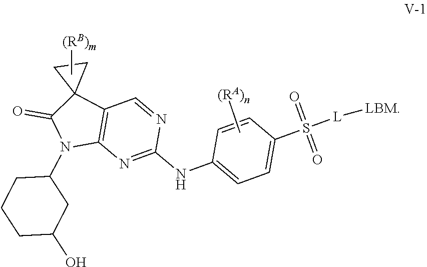

Embodiment 98. The compound of embodiment 18 or 20, or a pharmaceutically acceptable salt thereof, wherein the compound is of Formula V-1-A:

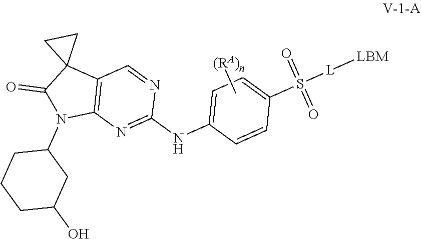

Embodiment 99. The compound of embodiment 18 or 20, or a pharmaceutically acceptable salt thereof, wherein the compound is of Formula V-1-B:

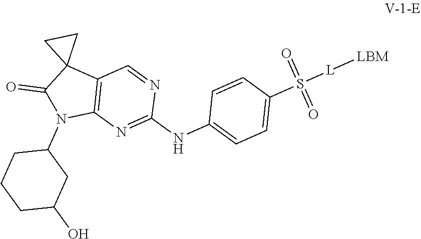

Embodiment 100. The compound of embodiment 18 or 20, or a pharmaceutically acceptable salt thereof, wherein the compound is of Formula V-1-C:

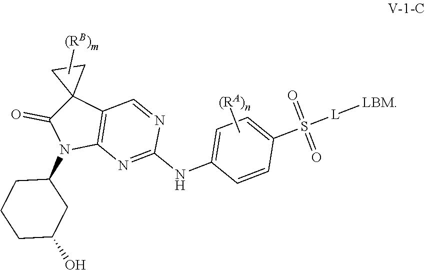

Embodiment 101. The compound of embodiment 18 or 20, or a pharmaceutically acceptable salt thereof, wherein the compound is of Formula V-1-D:

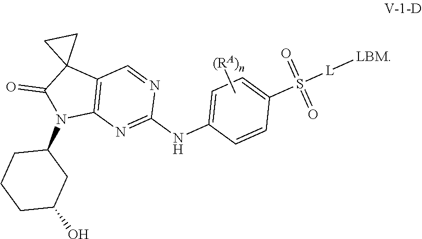

Embodiment 102. The compound of embodiment 18 or 20, or a pharmaceutically acceptable salt thereof, wherein the compound is of Formula V-1-E:

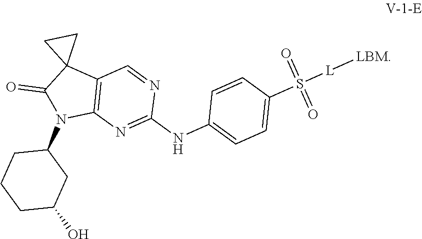

Embodiment 103. The compound of any one of embodiments 1-102, or a pharmaceutically acceptable salt thereof, wherein R^{A} is selected from halogen and —C₁₋₆ alkyl.

Embodiment 104. The compound of any one of embodiments 1-102, or a pharmaceutically acceptable salt thereof, wherein R^{A} is selected from —F and -Me.

Embodiment 105. The compound of any one of embodiments 1-102, or a pharmaceutically acceptable salt thereof, wherein R^{A} is —F.

Embodiment 106. The compound of any one of embodiments 1-102, or a pharmaceutically acceptable salt thereof, wherein the moiety represented by

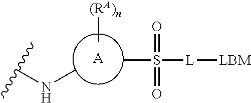

or by

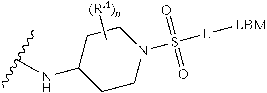

is selected from

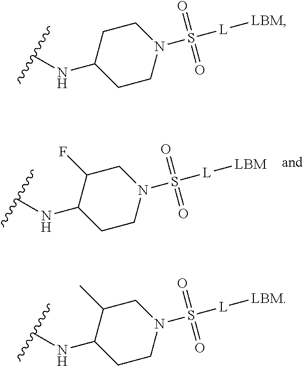

Embodiment 107. The compound of any one of embodiments 1-102, or a pharmaceutically acceptable salt thereof, wherein the moiety represented by

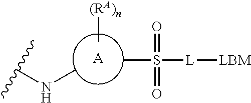

or by

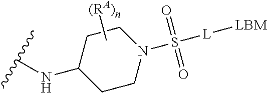

is

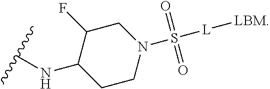

Embodiment 108. The compound of embodiment 106 or 107, or a pharmaceutically acceptable salt thereof, wherein the —NH— and F— are in a cis configuration.

Embodiment 109. The compound of embodiment 106 or 107, or a pharmaceutically acceptable salt thereof, wherein the —NH— and F— are in a trans configuration.

Embodiment 110. The compound of any one of embodiments 1-102, or a pharmaceutically acceptable salt thereof, wherein the moiety represented by

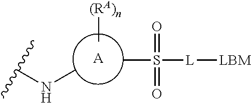

or by

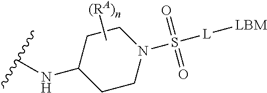

is

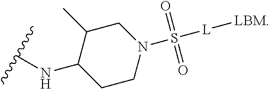

Embodiment 111. The compound of embodiment 106 or 110, or a pharmaceutically acceptable salt thereof, wherein the —NH— and Me- are in a cis configuration.

Embodiment 112. The compound of embodiment 106 or 110, or a pharmaceutically acceptable salt thereof, wherein the —NH— and Me- are in a trans configuration.

Embodiment 113. The compound of any one of embodiments 1-102, or a pharmaceutically acceptable salt thereof, wherein the moiety represented by

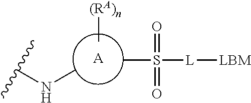

or by

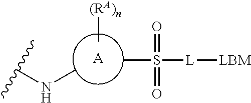

is selected from

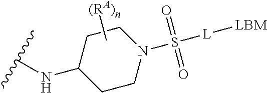

Embodiment 114. The compound of any one of embodiments 1-102, or a pharmaceutically acceptable salt thereof, wherein the moiety represented by

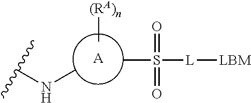

or by

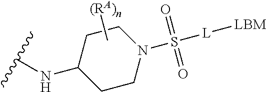

is selected from

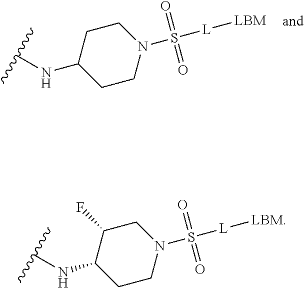

Embodiment 115. The compound of any one of embodiments 1-102, or a pharmaceutically acceptable salt thereof, wherein the moiety represented by

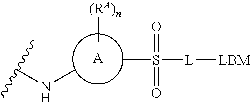

or by

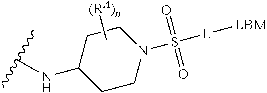

is

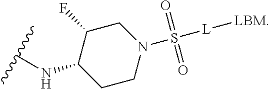

Embodiment 116. The compound of any one of embodiments 1-115, or a pharmaceutically acceptable salt thereof, wherein L is selected from:

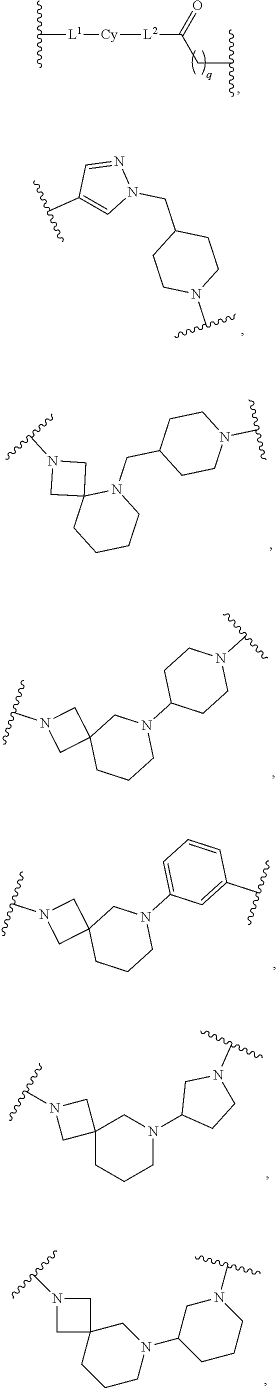

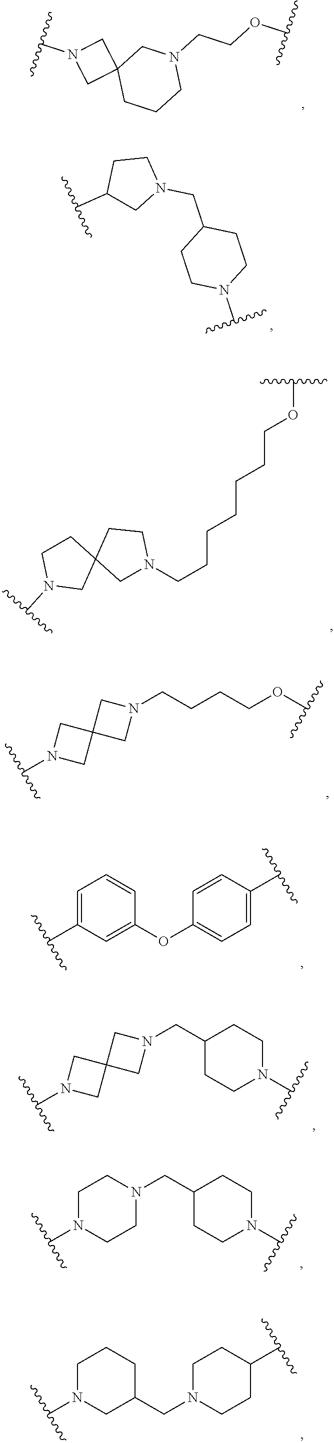

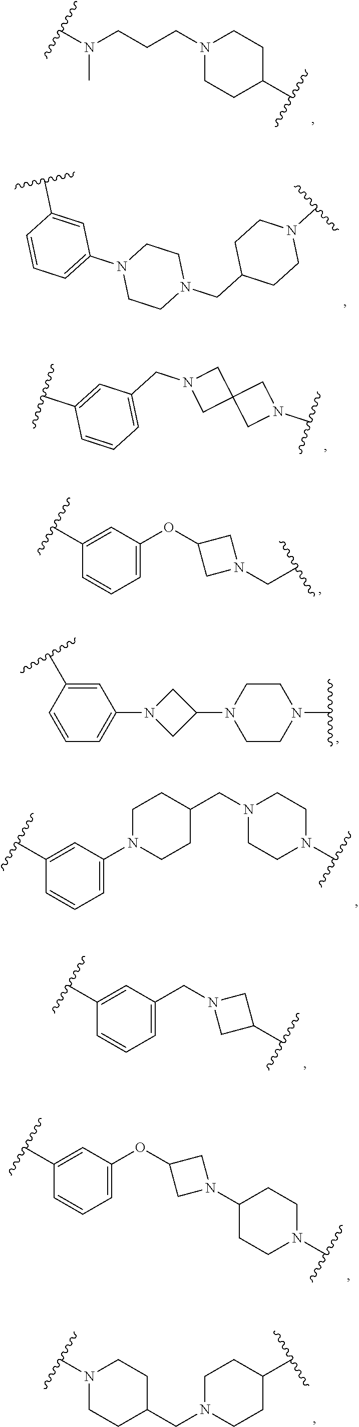

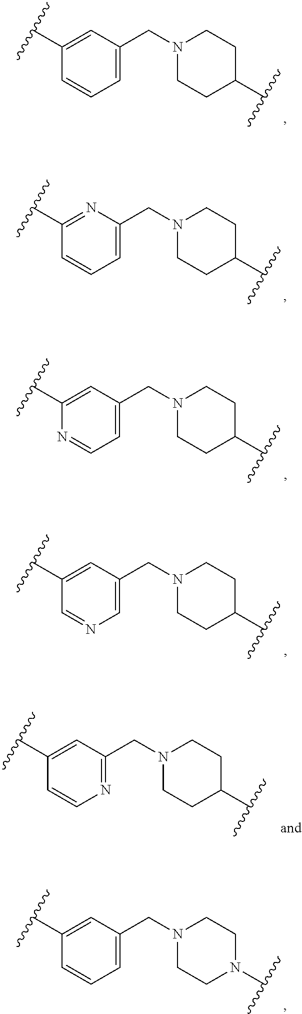 [SMILES: *c1cccc(CN2CCC(*)CC2)c1 ; *c1cccc(CN2CCC(*)CC2)n1 ; *c1cc(CN2CCC(*)CC2)ccn1 ; *c1cncc(CN2CCC(*)CC2)c1 ; *c1ccnc(CN2CCC(*)CC2)c1 ; *c1cccc(CN2CCN(*)CC2)c1]

and, each substituted with 0, 1, 2 or 3 instances of R⁷, wherein each R⁷ is independently selected from —C₁₋₄ alkyl and halo;
wherein the left side attachment point connects to the —S(O)₂— group and the right side attachment point connects to LBM;
L¹ and L² are each independently selected from a bond and —N(R′), wherein R′ is selected from H and C₁₋₆ alkyl; and
q is 0, 1, 2, 3, 4, 5, 6, 7, 8, 9 or 10.

Embodiment 117. The compound of embodiment 116, or a pharmaceutically acceptable salt thereof, wherein each R⁷ is independently selected from -Me, —ⁱPr and —F.

Embodiment 118. The compound of embodiment 116, or a pharmaceutically acceptable salt thereof, wherein each R⁷ is independently selected from -Me and —F.

Embodiment 119. The compound of any one of embodiments 1-115, or a pharmaceutically acceptable salt thereof, wherein L is selected from:

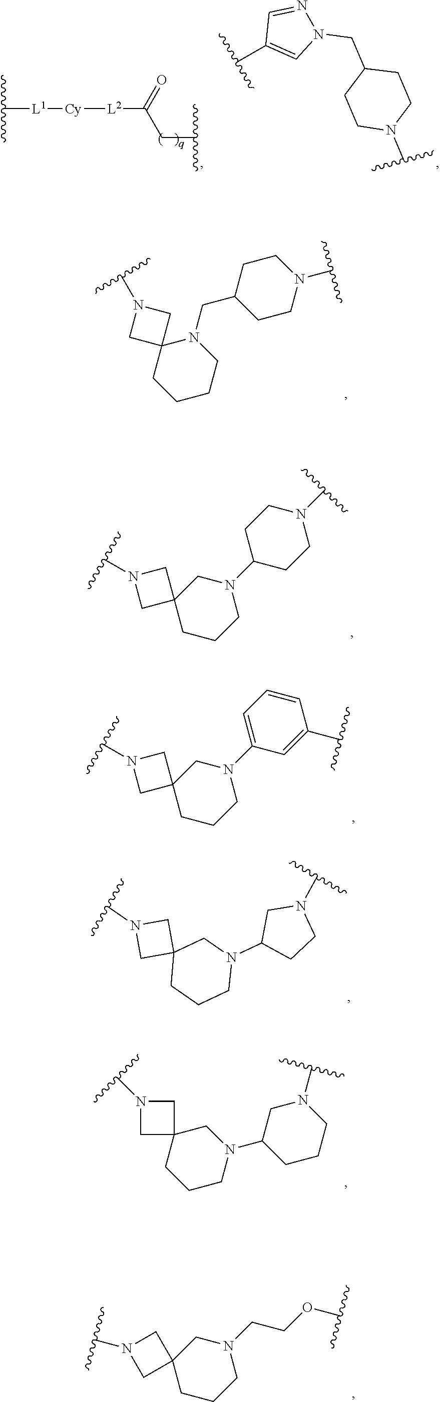

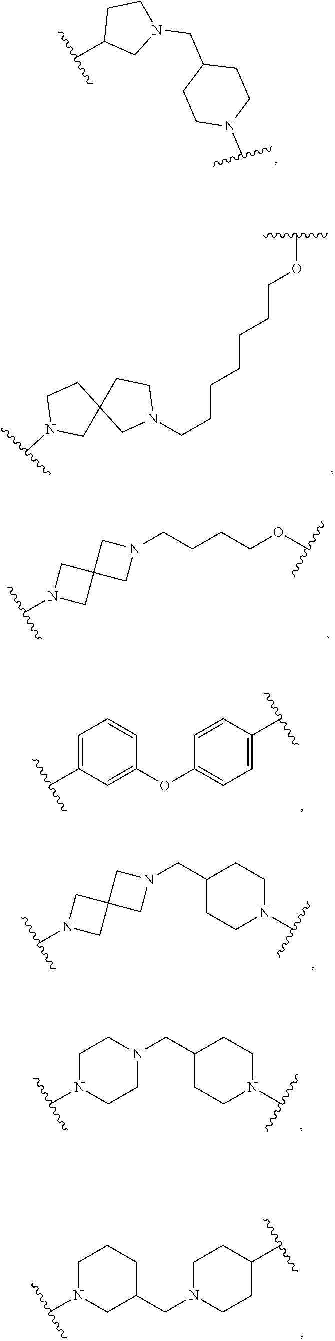

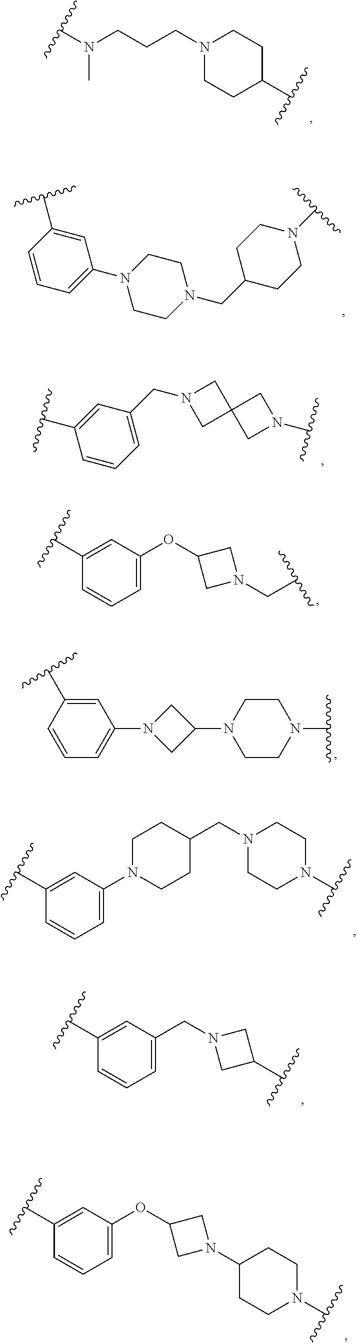

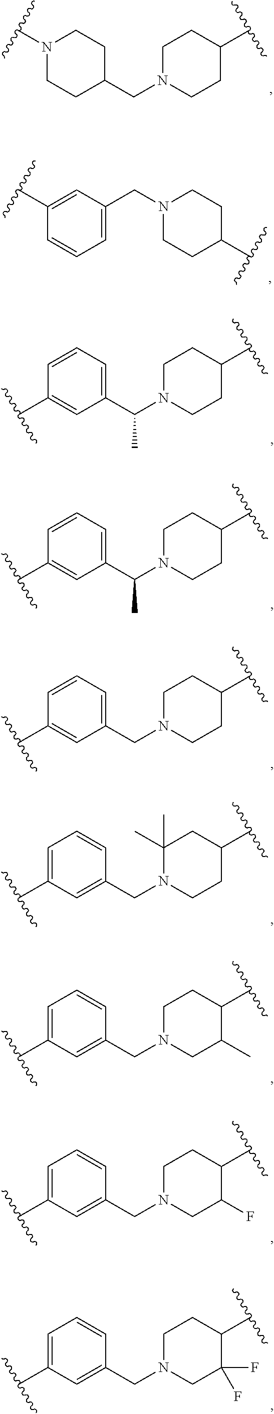

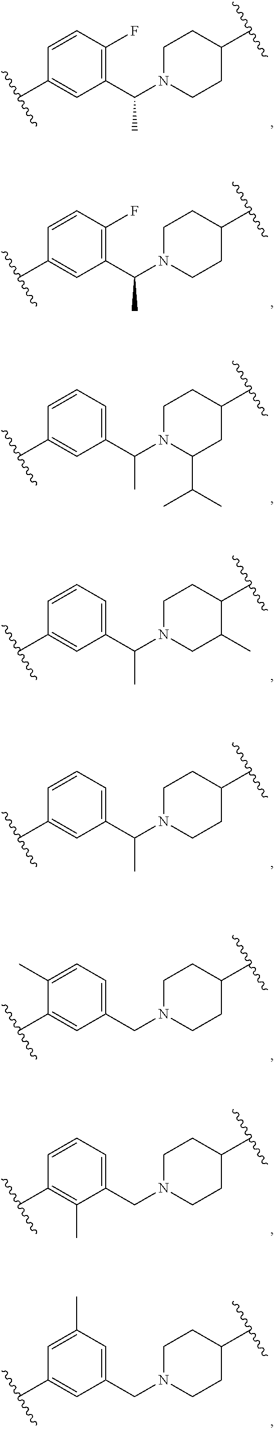

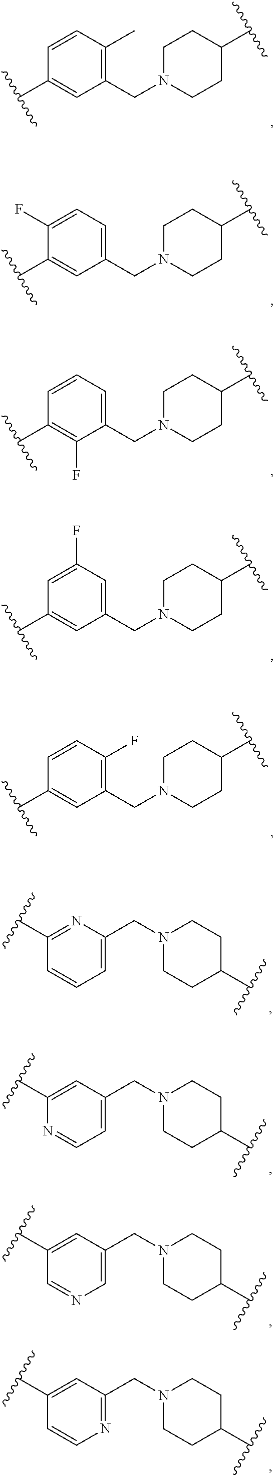

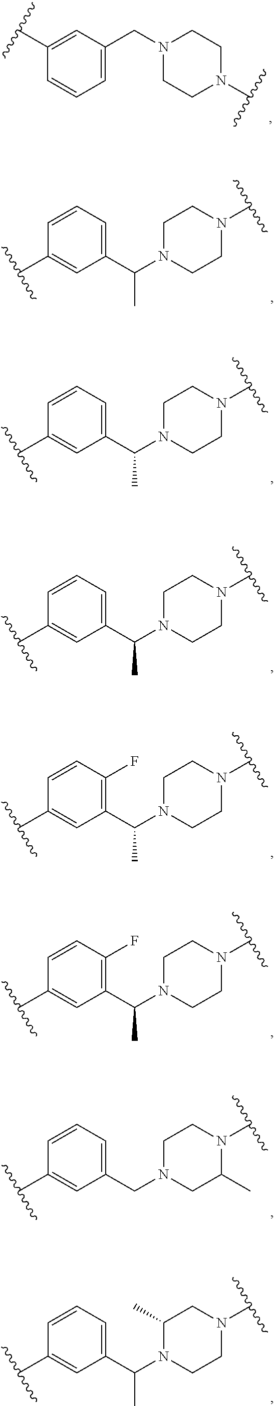

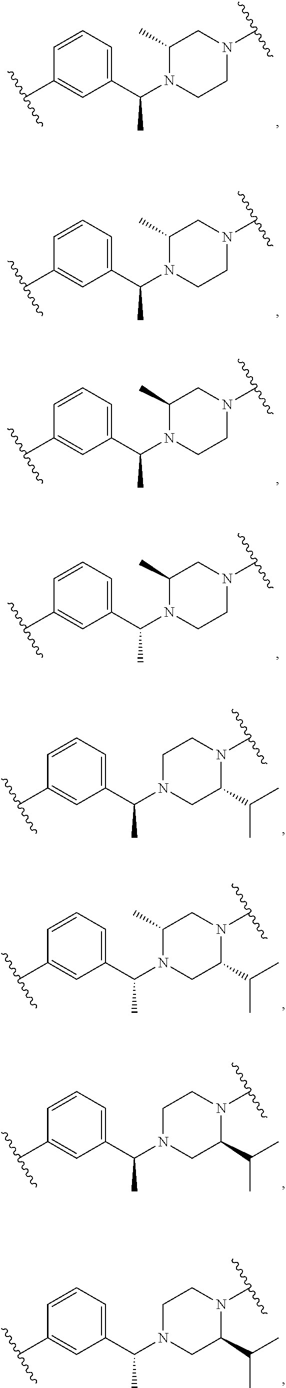

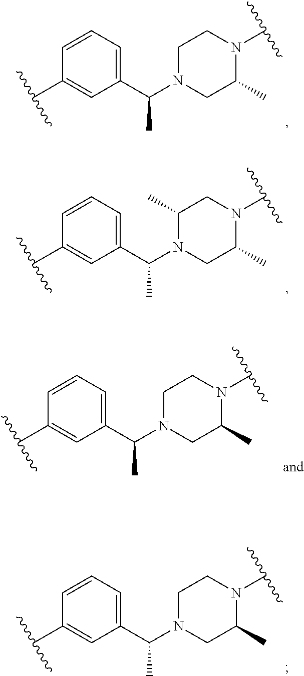 [SMILES: *c1cccc([C@H](C)N2CCN(*)[C@H](C)C2)c1 ; *c1cccc([C@@H](C)N2C[C@@H](C)N(*)C[C@H]2C)c1 ; *c1cccc([C@H](C)N2CCN(*)[C@@H](C)C2)c1 ; *c1cccc([C@@H](C)N2CCN(*)[C@@H](C)C2)c1]

wherein the left side attachment point connects to the —S(O)₂— group and the right side attachment point connects to LBM;
L¹ and L² are each independently selected from a bond and —N(R′), wherein R′ is selected from H and C₁₋₆ alkyl; and
q is 0, 1, 2, 3, 4, 5, 6, 7, 8, 9 or 10.

Embodiment 120. The compound of any one of embodiments 1-115, or a pharmaceutically acceptable salt thereof, wherein L is selected from:

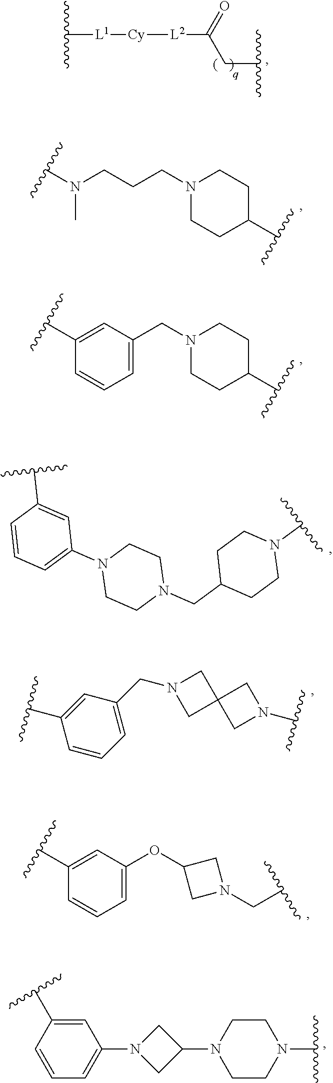

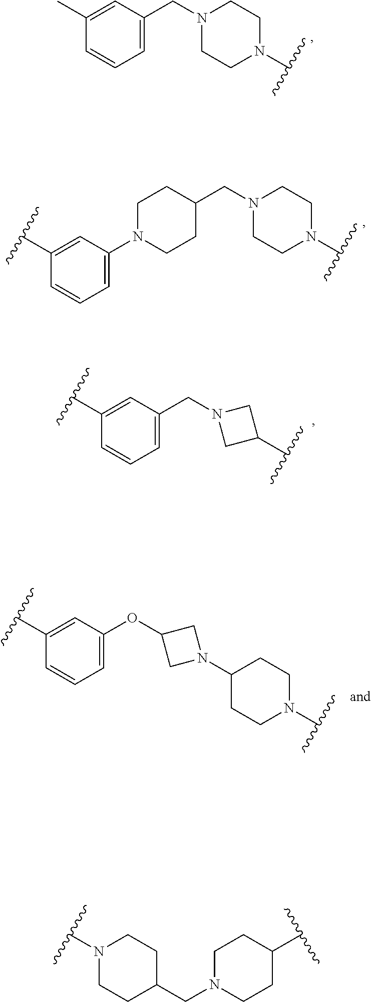 [SMILES: *N1CCN(Cc2cccc(C)c2)CC1 ; *c1cccc(N2CCC(CN3CCN(*)CC3)CC2)c1 ; *c1cccc(CN2CC(*)C2)c1 ; *c1cccc(OC2CN(C3CCN(*)CC3)C2)c1 ; *C1CCN(CC2CCN(*)CC2)CC1]

wherein the left side attachment point connects to the —S(O)₂— group and the right side attachment point connects to LBM;
L¹ and L² are each independently selected from a bond and —N(R′), wherein R′ is selected from H and C₁₋₆ alkyl; and
q is 0, 1, 2, 3, 4, 5, 6, 7, 8, 9 or 10.

Embodiment 121. The compound of any one of embodiments 1-115, or a pharmaceutically acceptable salt thereof, wherein L is selected from:

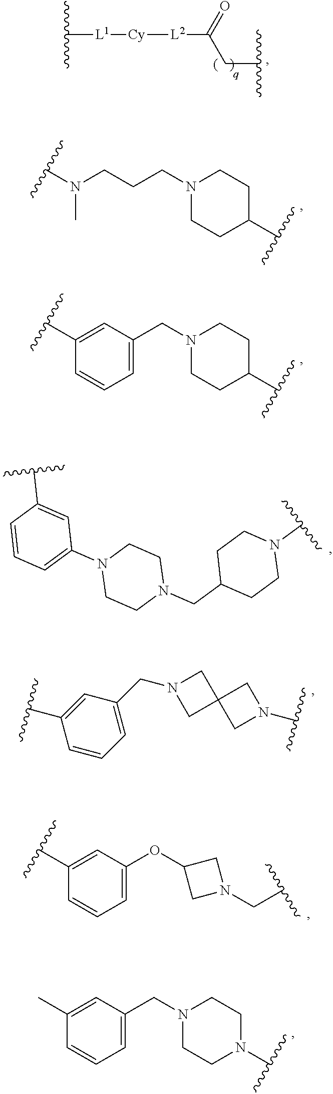

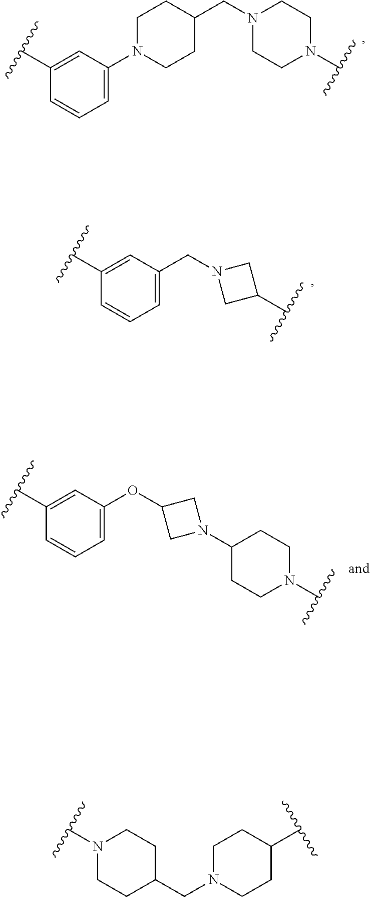 [SMILES: *c1cccc(N2CCC(CN3CCN(*)CC3)CC2)c1 ; *c1cccc(CN2CC(*)C2)c1 ; *c1cccc(OC2CN(C3CCN(*)CC3)C2)c1 ; *C1CCN(CC2CCN(*)CC2)CC1]

wherein the left side attachment point connects to the —S(O)₂— group and the right side attachment point connects to LBM;
L¹ and L² are each independently selected from a bond and —N(R′), wherein R′ is selected from H and C₁₋₆ alkyl; and
q is 0, 1, 2, 3, 4, 5, 6, 7, 8, 9 or 10.

Embodiment 122. The compound of any one of embodiments 116-118, or a pharmaceutically acceptable salt thereof, wherein L is selected from:

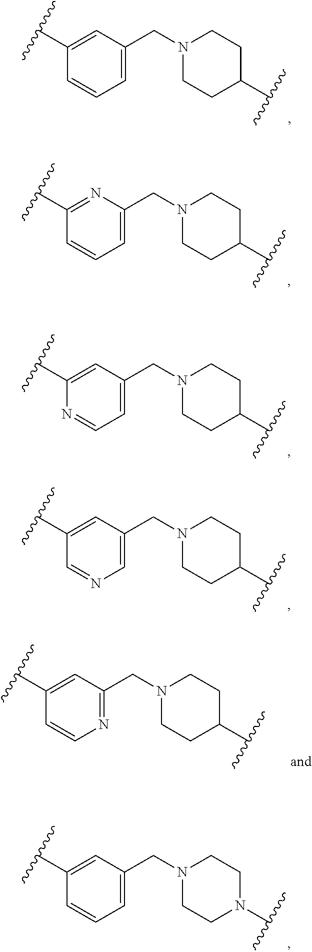 [SMILES: *c1cccc(CN2CCC(*)CC2)c1 ; *c1cccc(CN2CCC(*)CC2)n1 ; *c1cc(CN2CCC(*)CC2)ccn1 ; *c1cncc(CN2CCC(*)CC2)c1 ; *c1ccnc(CN2CCC(*)CC2)c1 ; *c1cccc(CN2CCN(*)CC2)c1]

each substituted with 0, 1, 2 or 3 instances of R⁷;
wherein the left side attachment point connects to the —S(O)₂— group and the right side attachment point connects to LBM.

Embodiment 123. The compound of any one of embodiments 116-118, or a pharmaceutically acceptable salt thereof, wherein L is

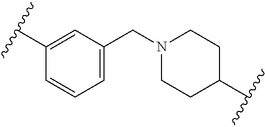 [SMILES: *c1cccc(CN2CCC(*)CC2)c1]

substituted with 0, 1, 2 or 3 instances of R⁷;
wherein the left side attachment point connects to the —S(O)₂— group and the right side attachment point connects to LBM.

Embodiment 124. The compound of any one of embodiments 116-118, or a pharmaceutically acceptable salt thereof, wherein L is

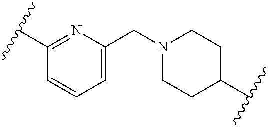 [SMILES: *c1cccc(CN2CCC(*)CC2)n1]

substituted with 0, 1, 2 or 3 instances of R⁷;
wherein the left side attachment point connects to the —S(O)₂— group and the right side attachment point connects to LBM.

Embodiment 125. The compound of any one of embodiments 116-118, or a pharmaceutically acceptable salt thereof, wherein L is

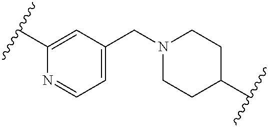 [SMILES: *c1cc(CN2CCC(*)CC2)ccn1]

substituted 0, 1, 2 or 3 instances of R⁷;
wherein the left side attachment point connects to the —S(O)₂— group and the right side attachment point connects to LBM.

Embodiment 126. The compound of any one of embodiments 116-118, or a pharmaceutically acceptable salt thereof, wherein L is

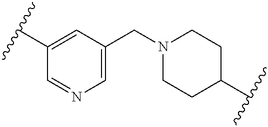 [SMILES: *c1cncc(CN2CCC(*)CC2)c1]

substituted with 0, 1, 2 or 3 instances of R⁷;
wherein the left side attachment point connects to the —S(O)₂— group and the right side attachment point connects to LBM.

Embodiment 127. The compound of any one of embodiments 116-118, or a pharmaceutically acceptable salt thereof, wherein L is

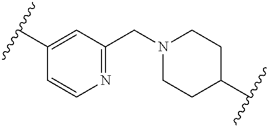 [SMILES: *c1ccnc(CN2CCC(*)CC2)c1]

substituted with 0, 1, 2 or 3 instances of R⁷;
wherein the left side attachment point connects to the —S(O)₂— group and the right side attachment point connects to LBM.

Embodiment 128. The compound of any one of embodiments 116-118, or a pharmaceutically acceptable salt thereof, wherein L is

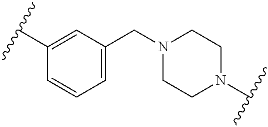 [SMILES: *c1cccc(CN2CCN(*)CC2)c1]

substituted with 0, 1, 2 or 3 instances of R⁷;
wherein the left side attachment point connects to the —S(O)₂— group and the right side attachment point connects to LBM.

Embodiment 129. The compound of any one of embodiments 1-115, or a pharmaceutically acceptable salt thereof, wherein L is

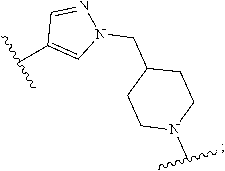 [SMILES: *c1cnn(CC2CCN(*)CC2)c1]

wherein the left side attachment point connects to the —S(O)₂— group and the right side attachment point connects to LBM.

Embodiment 130. The compound of any one of embodiments 1-115, or a pharmaceutically acceptable salt thereof, wherein L is

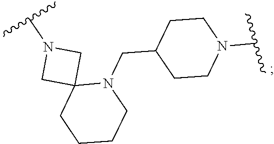 [SMILES: *N1CCC(CN2CCCCC23CN(*)C3)CC1]

wherein the left side attachment point connects to the —S(O)₂— group and the right side attachment point connects to LBM.

Embodiment 131. The compound of any one of embodiments 1-115, or a pharmaceutically acceptable salt thereof, wherein L is

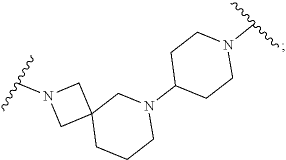 [SMILES: *N1CCC(N2CCCC3(CN(*)C3)C2)CC1]

wherein the left side attachment point connects to the —S(O)₂— group and the right side attachment point connects to LBM.

Embodiment 132. The compound of any one of embodiments 1-115, or a pharmaceutically acceptable salt thereof, wherein L is

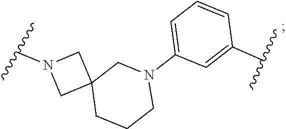 [SMILES: *c1cccc(N2CCCC3(CN(*)C3)C2)c1]

wherein the left side attachment point connects to the —S(O)₂— group and the right side attachment point connects to LBM.

Embodiment 133. The compound of any one of embodiments 1-115, or a pharmaceutically acceptable salt thereof, wherein L is

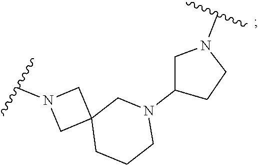 [SMILES: *N1CCC(N2CCCC3(CN(*)C3)C2)C1]

wherein the left side attachment point connects to the —S(O)₂— group and the right side attachment point connects to LBM.

Embodiment 134. The compound of any one of embodiments 1-115, or a pharmaceutically acceptable salt thereof, wherein L is

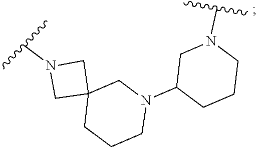 [SMILES: *N1CCCC(N2CCCC3(CN(*)C3)C2)C1]

wherein the left side attachment point connects to the —S(O)₂— group and the right side attachment point connects to LBM.

Embodiment 135. The compound of any one of embodiments 1-115, or a pharmaceutically acceptable salt thereof, wherein L is

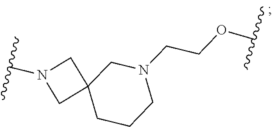 [SMILES: *OCCN1CCCC2(CN(*)C2)C1]

wherein the left side attachment point connects to the —S(O)₂— group and the right side attachment point connects to LBM.

Embodiment 136. The compound of any one of embodiments 1-115, or a pharmaceutically acceptable salt thereof, wherein L is

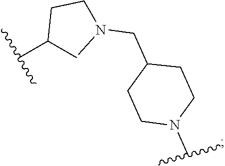 [SMILES: *C(C)CCN(C)CC1CCN(*)CC1]

wherein the left side attachment point connects to the —S(O)₂— group and the right side attachment point connects to LBM.

Embodiment 137. The compound of any one of embodiments 1-115, or a pharmaceutically acceptable salt thereof, wherein L is

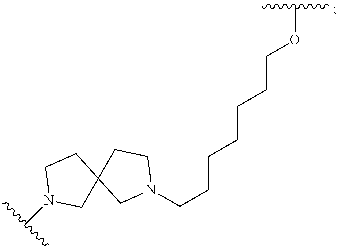 [SMILES: *OCCCCCCCN1CCC2(CCN(*)C2)C1]

wherein the left side attachment point connects to the —S(O)₂— group and the right side attachment point connects to LBM.

Embodiment 138. The compound of any one of embodiments 1-115, or a pharmaceutically acceptable salt thereof, wherein L is

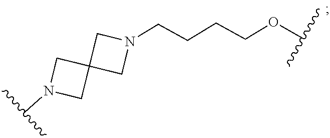 [SMILES: *OCCCCN1CC2(CN(*)C2)C1]

wherein the left side attachment point connects to the —S(O)₂— group and the right side attachment point connects to LBM.

Embodiment 139. The compound of any one of embodiments 1-115, or a pharmaceutically acceptable salt thereof, wherein L is

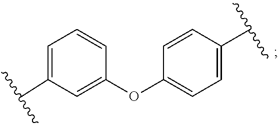 [SMILES: *c1ccc(Oc2cccc(*)c2)cc1]

wherein the left side attachment point connects to the —S(O)₂— group and the right side attachment point connects to LBM.

Embodiment 140. The compound of any one of embodiments 1-115, or a pharmaceutically acceptable salt thereof, wherein L is

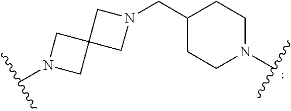 [SMILES: *N1CCC(CN2CC3(CN(*)C3)C2)CC1]

wherein the left side attachment point connects to the —S(O)₂— group and the right side attachment point connects to LBM.

Embodiment 141. The compound of any one of embodiments 1-115, or a pharmaceutically acceptable salt thereof, wherein L is

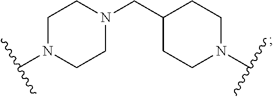 [SMILES: *N1CCC(CN2CCN(*)CC2)CC1]

wherein the left side attachment point connects to the —S(O)₂— group and the right side attachment point connects to LBM.

Embodiment 142. The compound of any one of embodiments 1-115, or a pharmaceutically acceptable salt thereof, wherein L is

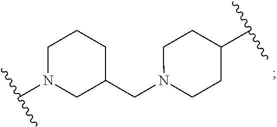 [SMILES: *C1CCN(CC2CCCN(*)C2)CC1]

wherein the left side attachment point connects to the —S(O)₂— group and the right side attachment point connects to LBM.

Embodiment 143. The compound of any one of embodiments 1-115, or a pharmaceutically acceptable salt thereof, wherein L is

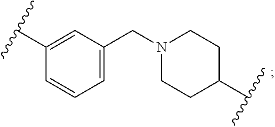 [SMILES: *c1cccc(CN2CCC(*)CC2)c1]

wherein the left side attachment point connects to the —S(O)₂— group and the right side attachment point connects to LBM.

Embodiment 144. The compound of any one of embodiments 1-115, or a pharmaceutically acceptable salt thereof, wherein L is

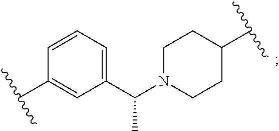 [SMILES: *c1cccc([C@@H](C)N2CCC(*)CC2)c1]

wherein the left side attachment point connects to the —S(O)₂— group and the right side attachment point connects to LBM.

Embodiment 145. The compound of any one of embodiments 1-115, or a pharmaceutically acceptable salt thereof, wherein L is

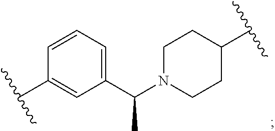 [SMILES: *c1cccc([C@H](C)N2CCC(*)CC2)c1]

wherein the left side attachment point connects to the —S(O)₂— group and the right side attachment point connects to LBM.

Embodiment 146. The compound of any one of embodiments 1-115, or a pharmaceutically acceptable salt thereof, wherein L is

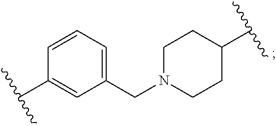 [SMILES: *c1cccc(CN2CCC(*)CC2)c1]

wherein the left side attachment point connects to the —S(O)₂— group and the right side attachment point connects to LBM.

Embodiment 147. The compound of any one of embodiments 1-115, or a pharmaceutically acceptable salt thereof, wherein L is

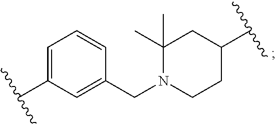 [SMILES: *c1cccc(CN2CCC(*)CC2(C)C)c1]

wherein the left side attachment point connects to the —S(O)₂— group and the right side attachment point connects to LBM.

Embodiment 148. The compound of any one of embodiments 1-115, or a pharmaceutically acceptable salt thereof, wherein L is

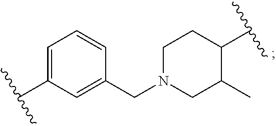 [SMILES: *c1cccc(CN2CCC(*)C(C)C2)c1]

wherein the left side attachment point connects to the —S(O)₂— group and the right side attachment point connects to LBM.

Embodiment 149. The compound of any one of embodiments 1-115, or a pharmaceutically acceptable salt thereof, wherein L is

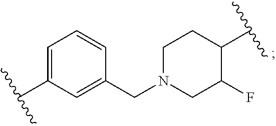 [SMILES: *c1cccc(CN2CCC(*)C(F)C2)c1]

wherein the left side attachment point connects to the —S(O)₂— group and the right side attachment point connects to LBM.

Embodiment 150. The compound of any one of embodiments 1-115, or a pharmaceutically acceptable salt thereof, wherein L is

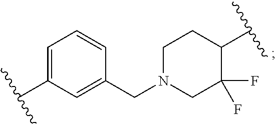 [SMILES: *c1cccc(CN2CCC(*)C(F)(F)C2)c1]

wherein the left side attachment point connects to the —S(O)₂— group and the right side attachment point connects to LBM.

Embodiment 151. The compound of any one of embodiments 1-115, or a pharmaceutically acceptable salt thereof, wherein L is

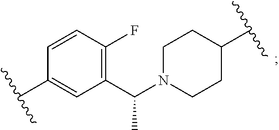 [SMILES: *c1ccc(F)c([C@@H](C)N2CCC(*)CC2)c1]

wherein the left side attachment point connects to the —S(O)₂— group and the right side attachment point connects to LBM.

Embodiment 152. The compound of any one of embodiments 1-115, or a pharmaceutically acceptable salt thereof, wherein L is

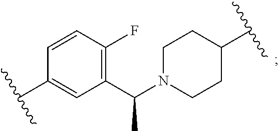 [SMILES: *c1ccc(F)c([C@H](C)N2CCC(*)CC2)c1]

wherein the left side attachment point connects to the —S(O)₂— group and the right side attachment point connects to LBM.

Embodiment 153. The compound of any one of embodiments 1-115, or a pharmaceutically acceptable salt thereof, wherein L is

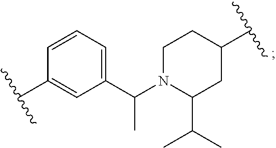 [SMILES: *c1cccc(C(C)N2CCC(*)CC2C(C)C)c1]

wherein the left side attachment point connects to the —S(O)₂— group and the right side attachment point connects to LBM.

Embodiment 154. The compound of any one of embodiments 1-115, or a pharmaceutically acceptable salt thereof, wherein L is

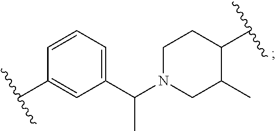 [SMILES: *c1cccc(C(C)N2CCC(*)C(C)C2)c1]

wherein the left side attachment point connects to the —S(O)₂— group and the right side attachment point connects to LBM.

Embodiment 155. The compound of any one of embodiments 1-115, or a pharmaceutically acceptable salt thereof, wherein L is

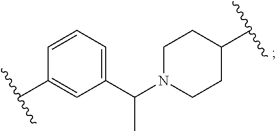 [SMILES: *c1cccc(C(C)N2CCC(*)CC2)c1]

wherein the left side attachment point connects to the —S(O)₂— group and the right side attachment point connects to LBM.

Embodiment 156. The compound of any one of embodiments 1-115, or a pharmaceutically acceptable salt thereof, wherein L is

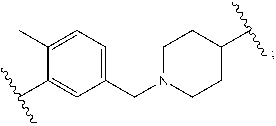 [SMILES: *c1cc(CN2CCC(*)CC2)ccc1C]

wherein the left side attachment point connects to the —S(O)₂— group and the right side attachment point connects to LBM.

Embodiment 157. The compound of any one of embodiments 1-115, or a pharmaceutically acceptable salt thereof, wherein L is

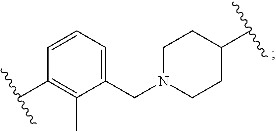 [SMILES: *c1cccc(CN2CCC(*)CC2)c1C]

wherein the left side attachment point connects to the —S(O)₂— group and the right side attachment point connects to LBM.

Embodiment 158. The compound of any one of embodiments 1-115, or a pharmaceutically acceptable salt thereof, wherein L is

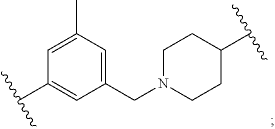 [SMILES: *c1cc(C)cc(CN2CCC(*)CC2)c1]

wherein the left side attachment point connects to the —S(O)₂— group and the right side attachment point connects to LBM.

Embodiment 159. The compound of any one of embodiments 1-115, or a pharmaceutically acceptable salt thereof, wherein L is

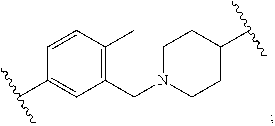 [SMILES: *c1ccc(C)c(CN2CCC(*)CC2)c1]

wherein the left side attachment point connects to the —S(O)₂— group and the right side attachment point connects to LBM.

Embodiment 160. The compound of any one of embodiments 1-115, or a pharmaceutically acceptable salt thereof, wherein L is

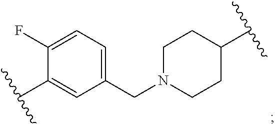 [SMILES: *c1cc(CN2CCC(*)CC2)ccc1F]

wherein the left side attachment point connects to the —S(O)₂— group and the right side attachment point connects to LBM.

Embodiment 161. The compound of any one of embodiments 1-115, or a pharmaceutically acceptable salt thereof, wherein L is

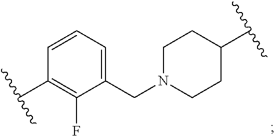 [SMILES: *c1cccc(CN2CCC(*)CC2)c1F]

wherein the left side attachment point connects to the —S(O)₂— group and the right side attachment point connects to LBM.

Embodiment 162. The compound of any one of embodiments 1-115, or a pharmaceutically acceptable salt thereof, wherein L is

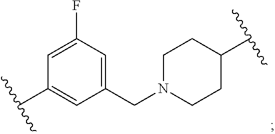 [SMILES: *c1cc(F)cc(CN2CCC(*)CC2)c1]

wherein the left side attachment point connects to the —S(O)₂— group and the right side attachment point connects to LBM.

Embodiment 163. The compound of any one of embodiments 1-115, or a pharmaceutically acceptable salt thereof, wherein L is

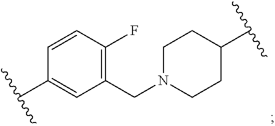 [SMILES: *c1ccc(F)c(CN2CCC(*)CC2)c1]

wherein the left side attachment point connects to the —S(O)₂— group and the right side attachment point connects to LBM.

Embodiment 164. The compound of any one of embodiments 1-115, or a pharmaceutically acceptable salt thereof, wherein L is

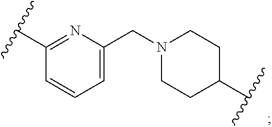 [SMILES: *c1cccc(CN2CCC(*)CC2)n1]

wherein the left side attachment point connects to the —S(O)₂— group and the right side attachment point connects to LBM.

Embodiment 165. The compound of any one of embodiments 1-115, or a pharmaceutically acceptable salt thereof, wherein L is

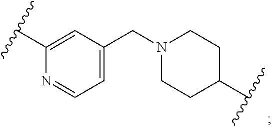 [SMILES: *c1cc(CN2CCC(*)CC2)ccn1]

wherein the left side attachment point connects to the —S(O)₂— group and the right side attachment point connects to LBM.

Embodiment 166. The compound of any one of embodiments 1-115, or a pharmaceutically acceptable salt thereof, wherein L is

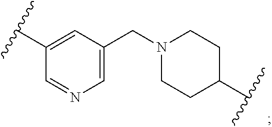 [SMILES: *c1cncc(CN2CCC(*)CC2)c1]

wherein the left side attachment point connects to the —S(O)₂— group and the right side attachment point connects to LBM.

Embodiment 167. The compound of any one of embodiments 1-115, or a pharmaceutically acceptable salt thereof, wherein L is

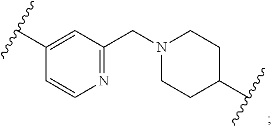 [SMILES: *c1ccnc(CN2CCC(*)CC2)c1]

wherein the left side attachment point connects to the —S(O)₂— group and the right side attachment point connects to LBM.

Embodiment 168. The compound of any one of embodiments 1-115, or a pharmaceutically acceptable salt thereof, wherein L is

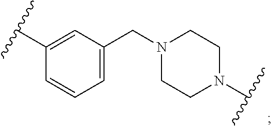 [SMILES: *c1cccc(CN2CCN(*)CC2)c1]

wherein the left side attachment point connects to the —S(O)₂— group and the right side attachment point connects to LBM.

Embodiment 169. The compound of any one of embodiments 1-115, or a pharmaceutically acceptable salt thereof, wherein L is

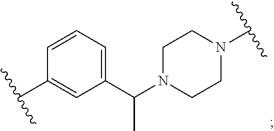 [SMILES: *c1cccc(C(C)N2CCN(*)CC2)c1]

wherein the left side attachment point connects to the —S(O)₂— group and the right side attachment point connects to LBM.

Embodiment 170. The compound of any one of embodiments 1-115, or a pharmaceutically acceptable salt thereof, wherein L is

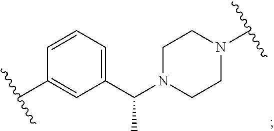 [SMILES: *c1cccc([C@@H](C)N2CCN(*)CC2)c1]

wherein the left side attachment point connects to the —S(O)₂— group and the right side attachment point connects to LBM.

Embodiment 171. The compound of any one of embodiments 1-115, or a pharmaceutically acceptable salt thereof, wherein L is

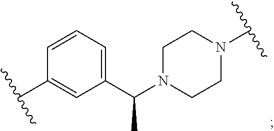 [SMILES: *c1cccc([C@H](C)N2CCN(*)CC2)c1]

wherein the left side attachment point connects to the —S(O)₂— group and the right side attachment point connects to LBM.

Embodiment 172. The compound of any one of embodiments 1-115, or a pharmaceutically acceptable salt thereof, wherein L is

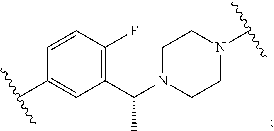 [SMILES: *c1ccc(F)c([C@@H](C)N2CCN(*)CC2)c1]

wherein the left side attachment point connects to the —S(O)₂— group and the right side attachment point connects to LBM.

Embodiment 173. The compound of any one of embodiments 1-115, or a pharmaceutically acceptable salt thereof, wherein L is

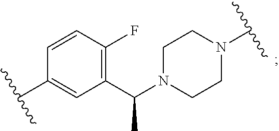 [SMILES: *c1ccc(F)c([C@H](C)N2CCN(*)CC2)c1]

wherein the left side attachment point connects to the —S(O)₂— group and the right side attachment point connects to LBM.

Embodiment 174. The compound of any one of embodiments 1-115, or a pharmaceutically acceptable salt thereof, wherein L is

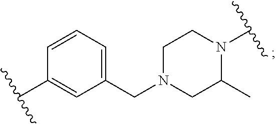 [SMILES: *c1cccc(CN2CCN(*)C(C)C2)c1]

wherein the left side attachment point connects to the —S(O)₂— group and the right side attachment point connects to LBM.

Embodiment 175. The compound of any one of embodiments 1-115, or a pharmaceutically acceptable salt thereof, wherein L is

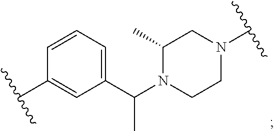 [SMILES: *c1cccc(C(C)N2CCN(*)C[C@H]2C)c1]

wherein the left side attachment point connects to the —S(O)₂— group and the right side attachment point connects to LBM.

Embodiment 176. The compound of any one of embodiments 1-115, or a pharmaceutically acceptable salt thereof, wherein L is

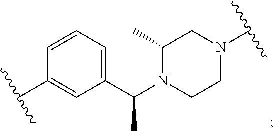 [SMILES: *c1cccc([C@H](C)N2CCN(*)C[C@H]2C)c1]

wherein the left side attachment point connects to the —S(O)₂— group and the right side attachment point connects to LBM.

Embodiment 177. The compound of any one of embodiments 1-115, or a pharmaceutically acceptable salt thereof, wherein L is

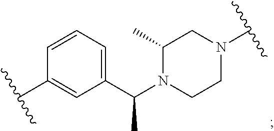 [SMILES: *c1cccc([C@H](C)N2CCN(*)C[C@H]2C)c1]

wherein the left side attachment point connects to the —S(O)₂— group and the right side attachment point connects to LBM.

Embodiment 178. The compound of any one of embodiments 1-115, or a pharmaceutically acceptable salt thereof, wherein L is

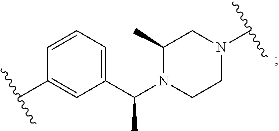 [SMILES: *c1cccc([C@H](C)N2CCN(*)C[C@@H]2C)c1]

wherein the left side attachment point connects to the —S(O)₂— group and the right side attachment point connects to LBM.

Embodiment 179. The compound of any one of embodiments 1-115, or a pharmaceutically acceptable salt thereof, wherein L is

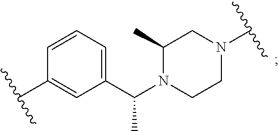 [SMILES: *c1cccc([C@@H](C)N2CCN(*)C[C@@H]2C)c1]

wherein the left side attachment point connects to the —S(O)₂— group and the right side attachment point connects to LBM.

Embodiment 180. The compound of any one of embodiments 1-115, or a pharmaceutically acceptable salt thereof, wherein L is

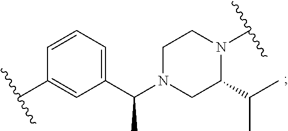 [SMILES: *c1cccc([C@H](C)N2CCN(*)[C@H](C(C)C)C2)c1]

wherein the left side attachment point connects to the —S(O)₂— group and the right side attachment point connects to LBM.

Embodiment 181. The compound of any one of embodiments 1-115, or a pharmaceutically acceptable salt thereof, wherein L is

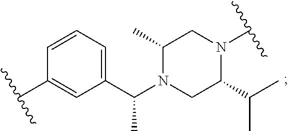 [SMILES: *c1cccc([C@@H](C)N2C[C@@H](C(C)C)N(*)C[C@H]2C)c1]

wherein the left side attachment point connects to the —S(O)₂— group and the right side attachment point connects to LBM.

Embodiment 182. The compound of any one of embodiments 1-115, or a pharmaceutically acceptable salt thereof, wherein L is

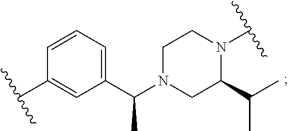 [SMILES: *c1cccc([C@H](C)N2CCN(*)[C@@H](C(C)C)C2)c1]

wherein the left side attachment point connects to the —S(O)₂— group and the right side attachment point connects to LBM.

Embodiment 183. The compound of any one of embodiments 1-115, or a pharmaceutically acceptable salt thereof, wherein L is

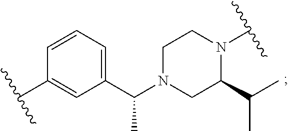 [SMILES: *c1cccc([C@@H](C)N2CCN(*)[C@@H](C(C)C)C2)c1]

wherein the left side attachment point connects to the —S(O)₂— group and the right side attachment point connects to LBM.

Embodiment 184. The compound of any one of embodiments 1-115, or a pharmaceutically acceptable salt thereof, wherein L is

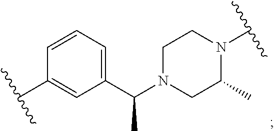 [SMILES: *c1cccc([C@H](C)N2CCN(*)[C@H](C)C2)c1]

wherein the left side attachment point connects to the —S(O)₂— group and the right side attachment point connects to LBM.

Embodiment 185. The compound of any one of embodiments 1-115, or a pharmaceutically acceptable salt thereof, wherein L is

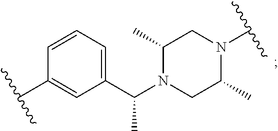 [SMILES: *c1cccc([C@@H](C)N2C[C@@H](C)N(*)C[C@H]2C)c1]

wherein the left side attachment point connects to the —S(O)₂— group and the right side attachment point connects to LBM.

Embodiment 186. The compound of any one of embodiments 1-115, or a pharmaceutically acceptable salt thereof, wherein L is

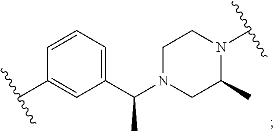 [SMILES: *c1cccc([C@H](C)N2CCN(*)[C@@H](C)C2)c1]

wherein the left side attachment point connects to the —S(O)₂— group and the right side attachment point connects to LBM.

Embodiment 187. The compound of any one of embodiments 1-115, or a pharmaceutically acceptable salt thereof, wherein L is

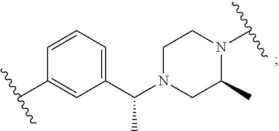 [SMILES: *c1cccc([C@@H](C)N2CCN(*)[C@@H](C)C2)c1]

wherein the left side attachment point connects to the —S(O)₂— group and the right side attachment point connects to LBM.

Embodiment 188. The compound of any one of embodiments 1-115, or a pharmaceutically acceptable salt thereof, wherein L is

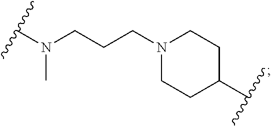 [SMILES: *C1CCN(CCCN(*)C)CC1]

wherein the left side attachment point connects to the —S(O)₂— group and the right side attachment point connects to LBM.

Embodiment 189. The compound of any one of embodiments 1-115, or a pharmaceutically acceptable salt thereof, wherein L is

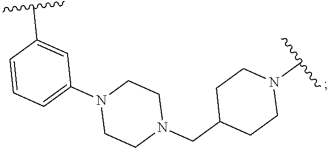 [SMILES: *c1cccc(N2CCN(CC3CCN(*)CC3)CC2)c1]

wherein the left side attachment point connects to the —S(O)₂— group and the right side attachment point connects to LBM.

Embodiment 190. The compound of any one of embodiments 1-115, or a pharmaceutically acceptable salt thereof, wherein L is

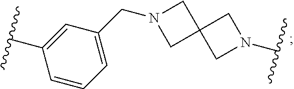 [SMILES: *c1cccc(CN2CC3(CN(*)C3)C2)c1]

wherein the left side attachment point connects to the —S(O)₂— group and the right side attachment point connects to LBM.

Embodiment 191. The compound of any one of embodiments 1-115, or a pharmaceutically acceptable salt thereof, wherein L is

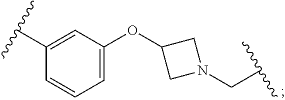 [SMILES: *CN1CC(Oc2cccc(*)c2)C1]

wherein the left side attachment point connects to the —S(O)₂— group and the right side attachment point connects to LBM.

Embodiment 192. The compound of any one of embodiments 1-115, or a pharmaceutically acceptable salt thereof, wherein L is

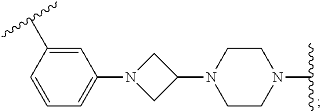 [SMILES: *c1cccc(N2CC(N3CCN(*)CC3)C2)c1]

wherein the left side attachment point connects to the —S(O)₂— group and the right side attachment point connects to LBM.

Embodiment 193. The compound of any one of embodiments 1-115, or a pharmaceutically acceptable salt thereof, wherein L is

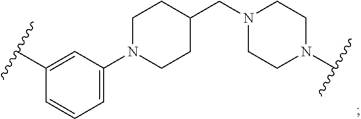 [SMILES: *c1cccc(N2CCC(CN3CCN(*)CC3)CC2)c1]

wherein the left side attachment point connects to the —S(O)₂— group and the right side attachment point connects to LBM.

Embodiment 194. The compound of any one of embodiments 1-115, or a pharmaceutically acceptable salt thereof, wherein L is

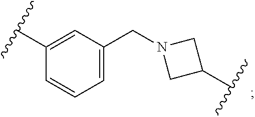 [SMILES: *c1cccc(CN2CC(*)C2)c1]

wherein the left side attachment point connects to the —S(O)₂— group and the right side attachment point connects to LBM.

Embodiment 195. The compound of any one of embodiments 1-115, or a pharmaceutically acceptable salt thereof, wherein L is

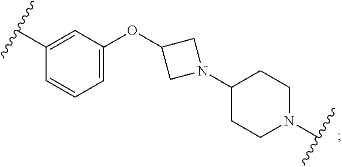 [SMILES: *c1cccc(OC2CN(C3CCN(*)CC3)C2)c1]

wherein the left side attachment point connects to the —S(O)₂— group and the right side attachment point connects to LBM.

Embodiment 196. The compound of any one of embodiments 1-115, or a pharmaceutically acceptable salt thereof, wherein L is

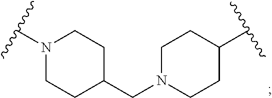 [SMILES: *C1CCN(CC2CCN(*)CC2)CC1]

wherein the left side attachment point connects to the —S(O)₂— group and the right side attachment point connects to LBM.

Embodiment 197. The compound of any one of embodiments 1-115, or a pharmaceutically acceptable salt thereof, wherein L is

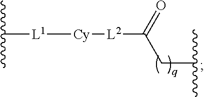

wherein the left side attachment point connects to the —S(O)₂— group and the right side attachment point connects to LBM.

Embodiment 198. The compound of any one of embodiments 1-121 and 197, or a pharmaceutically acceptable salt thereof, wherein Cy is selected from:

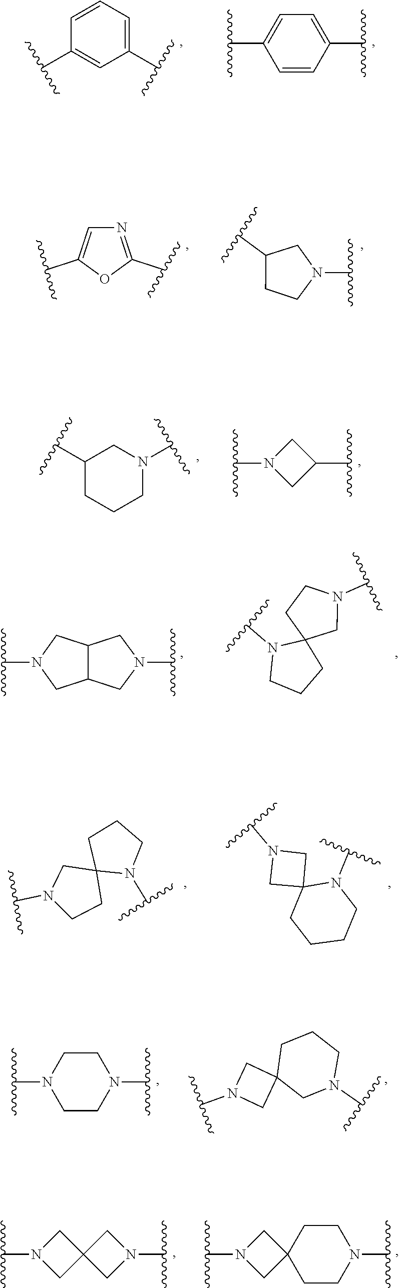

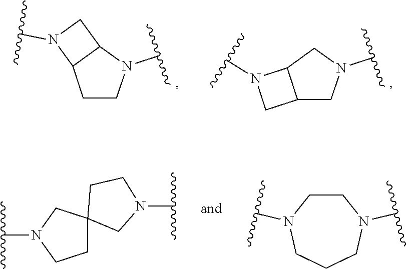 [SMILES: *N1CCC2C1CN2* ; *N1CC2CN(*)C2C1 ; *N1CCC2(CCN(*)C2)C1 ; *N1CCCN(*)CC1]

each substituted with 0, 1, 2, 3, or 4 instances of R^{C} wherein the left side attachment point connects to L¹ group and the right side attachment point connects to L².

Embodiment 199. The compound of any one of embodiments 1-121, 197 and 198, or a pharmaceutically acceptable salt thereof, wherein Cy is selected from:

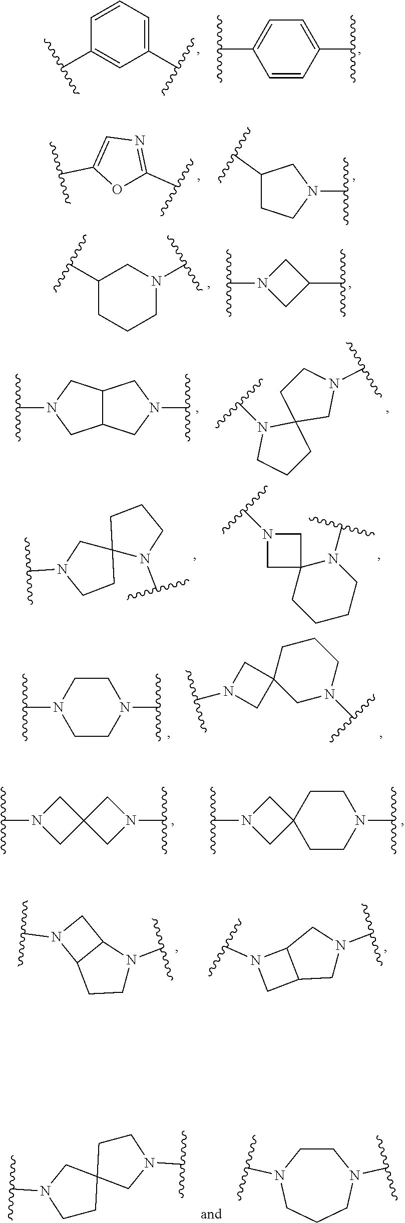

each not further substituted, wherein the left side attachment point connects to L¹ group and the right side attachment point connects to L².

Embodiment 200. The compound of any one of embodiments 1-121, 197 and 198, or a pharmaceutically acceptable salt thereof, wherein Cy is selected from:

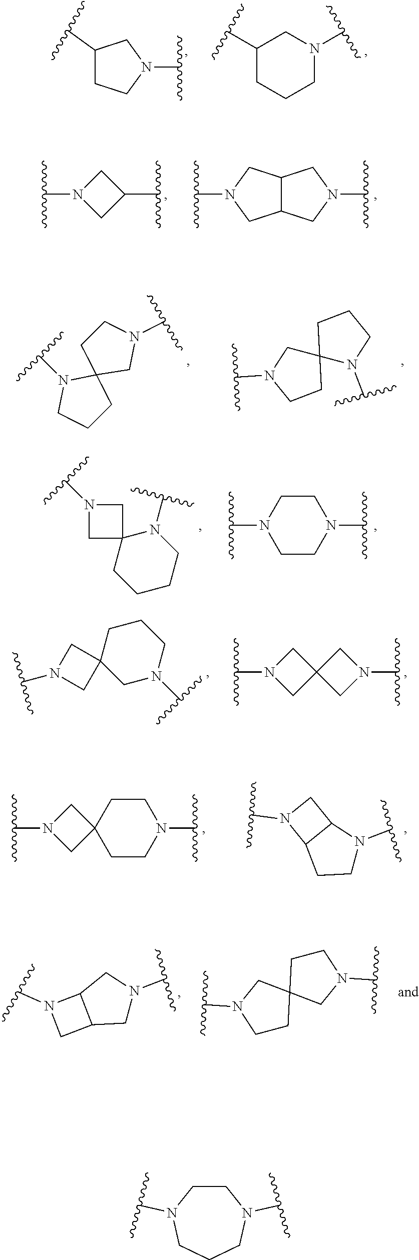

each not further substituted, wherein the left side attachment point connects to L¹ group and the right side attachment point connects to L².

Embodiment 201. The compound of any one of embodiments 1-121, 197 and 198, or a pharmaceutically acceptable salt thereof, wherein Cy is

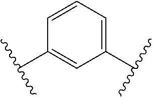 [SMILES: *c1cccc(*)c1]

wherein the left side attachment point connects to L¹ group and the right side attachment point connects to L².

Embodiment 202. The compound of any one of embodiments 1-121, 197 and 198, or a pharmaceutically acceptable salt thereof, wherein Cy is

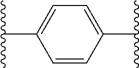 [SMILES: *c1ccc(*)cc1]

wherein the left side attachment point connects to L¹ group and the right side attachment point connects to L².

Embodiment 203. The compound of any one of embodiments 1-121, 197 and 198, or a pharmaceutically acceptable salt thereof, wherein Cy is

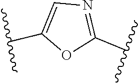 [SMILES: *c1cnc(*)o1]

wherein the left side attachment point connects to L¹ group and the right side attachment point connects to L².

Embodiment 204. The compound of any one of embodiments 1-121, 197 and 198, or a pharmaceutically acceptable salt thereof, wherein Cy is

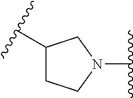 [SMILES: *C1CCN(*)C1]

wherein the left side attachment point connects to L¹ group and the right side attachment point connects to L².

Embodiment 205. The compound of any one of embodiments 1-121, 197 and 198, or a pharmaceutically acceptable salt thereof, wherein Cy is

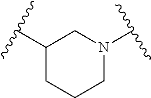 [SMILES: *C1CCCN(*)C1]

wherein the left side attachment point connects to L¹ group and the right side attachment point connects to L².

Embodiment 206. The compound of any one of embodiments 1-121, 197 and 198, or a pharmaceutically acceptable salt thereof, wherein Cy is

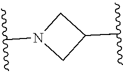 [SMILES: *C1CN(*)C1]

wherein the left side attachment point connects to L¹ group and the right side attachment point connects to L².

Embodiment 207. The compound of any one of embodiments 1-121, 197 and 198, or a pharmaceutically acceptable salt thereof, wherein Cy is

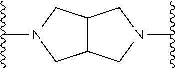 [SMILES: *N1CC2CN(*)CC2C1]

wherein the left side attachment point connects to L¹ group and the right side attachment point connects to L².

Embodiment 208. The compound of any one of embodiments 1-121, 197 and 198, or a pharmaceutically acceptable salt thereof, wherein Cy is

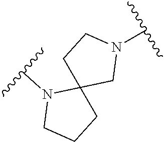 [SMILES: *N1CCC2(CCCN2*)C1]

wherein the left side attachment point connects to L¹ group and the right side attachment point connects to L².

Embodiment 209. The compound of any one of embodiments 1-121, 197 and 198, or a pharmaceutically acceptable salt thereof, wherein Cy is

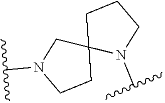 [SMILES: *N1CCC2(CCCN2*)C1]

wherein the left side attachment point connects to L¹ group and the right side attachment point connects to L².

Embodiment 210. The compound of any one of embodiments 1-121, 197 and 198, or a pharmaceutically acceptable salt thereof, wherein Cy is

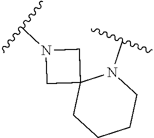 [SMILES: *N1CC2(CCCCN2*)C1]

wherein the left side attachment point connects to L¹ group and the right side attachment point connects to L².

Embodiment 211. The compound of any one of embodiments 1-121, 197 and 198, or a pharmaceutically acceptable salt thereof, wherein Cy is

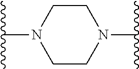 [SMILES: *N1CCN(*)CC1]

wherein the left side attachment point connects to L¹ group and the right side attachment point connects to L².

Embodiment 212. The compound of any one of embodiments 1-121, 197 and 198, or a pharmaceutically acceptable salt thereof, wherein Cy is

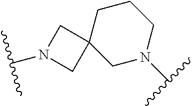 [SMILES: *N1CCCC2(C1)CN(*)C2]

wherein the left side attachment point connects to L¹ group and the right side attachment point connects to L².

Embodiment 213. The compound of any one of embodiments 1-121, 197 and 198, or a pharmaceutically acceptable salt thereof, wherein Cy is

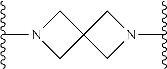 [SMILES: *N1CC2(C1)CN(*)C2]

wherein the left side attachment point connects to L¹ group and the right side attachment point connects to L².

Embodiment 214. The compound of any one of embodiments 1-121, 197 and 198, or a pharmaceutically acceptable salt thereof, wherein Cy is

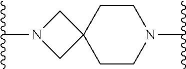 [SMILES: *N1CCC2(CC1)CN(*)C2]

wherein the left side attachment point connects to L¹ group and the right side attachment point connects to L².

Embodiment 215. The compound of any one of embodiments 1-121, 197 and 198, or a pharmaceutically acceptable salt thereof, wherein Cy is

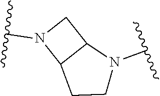 [SMILES: *N1CCC2C1CN2*]

wherein the left side attachment point connects to L¹ group and the right side attachment point connects to L².

Embodiment 216. The compound of any one of embodiments 1-121, 197 and 198, or a pharmaceutically acceptable salt thereof, wherein Cy is

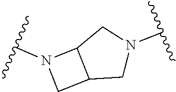 [SMILES: *N1CC2CN(*)C2C1]

wherein the left side attachment point connects to L¹ group and the right side attachment point connects to L².

Embodiment 217. The compound of any one of embodiments 1-121, 197 and 198, or a pharmaceutically acceptable salt thereof, wherein Cy is

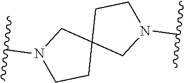 [SMILES: *N1CCC2(CCN(*)C2)C1]

wherein the left side attachment point connects to L¹ group and the right side attachment point connects to L².

Embodiment 218. The compound of any one of embodiments 1-121, 197 and 198, or a pharmaceutically acceptable salt thereof, wherein Cy is

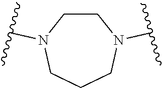 [SMILES: *N1CCCN(*)CC1]

wherein the left side attachment point connects to L¹ group and the right side attachment point connects to L².

Embodiment 219. The compound of any one of embodiments 116-121 and 197-218, wherein L¹ and L² are both bonds.

Embodiment 220. The compound of any one of embodiments 116-121 and 197-218, wherein L¹ is a bond and L² is a bond or N(R′)—.

Embodiment 221. The compound of any one of embodiments 116-121 and 197-218, wherein L¹ is a bond and L² is a bond or —NH—.

Embodiment 222. The compound of any one of embodiments 116-121 and 197, or a pharmaceutically acceptable salt thereof, wherein

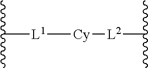

is selected from

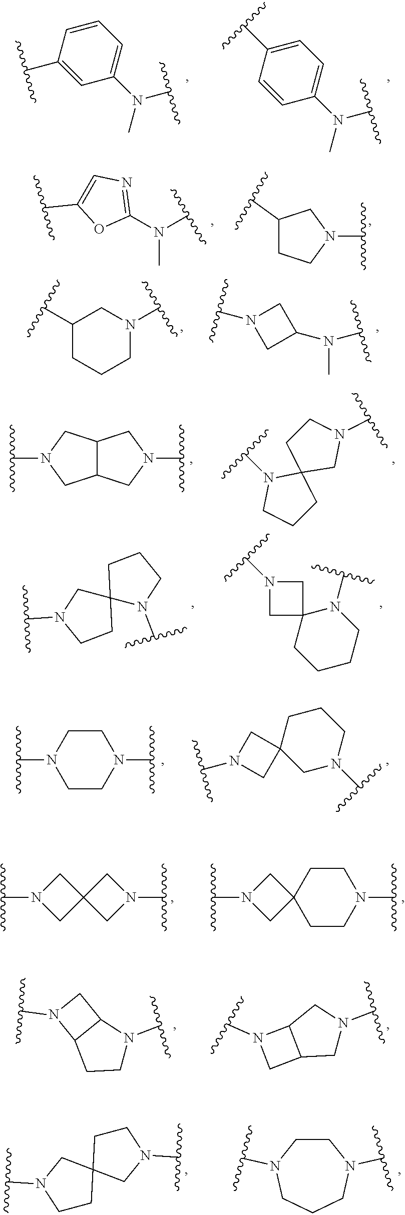

wherein the left attachment point connects to —S(O)₂— and the right attachment point connects to LBM.

Embodiment 223. The compound of any one of embodiments 116-121 and 197, or a pharmaceutically acceptable salt thereof, wherein

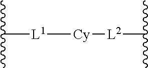

is selected from

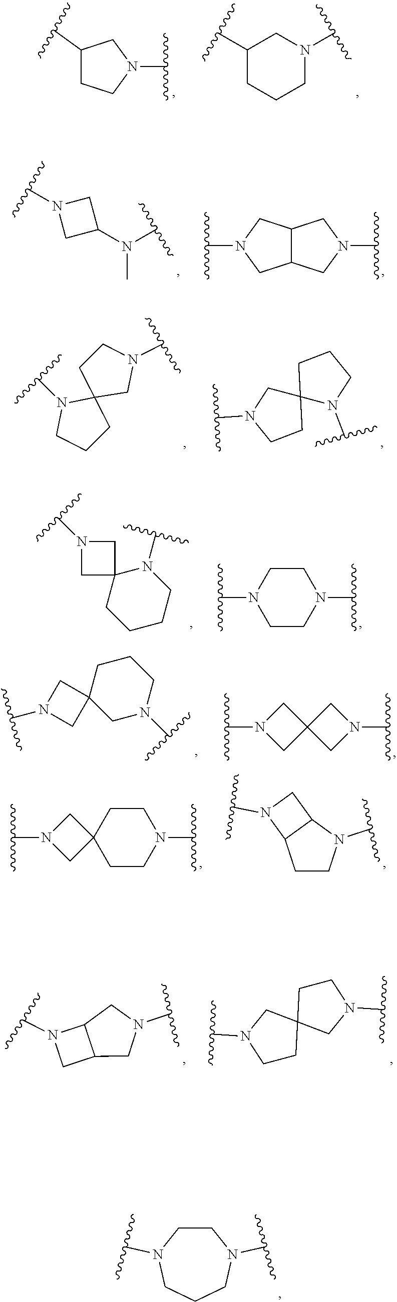

wherein the left attachment point connects to —S(O)₂— and the right attachment point connects to LBM.

Embodiment 224. The compound of any one of embodiments 116-121 and 197, or a pharmaceutically acceptable salt thereof, wherein

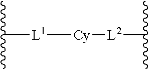

is

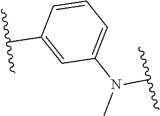 [SMILES: *c1cccc(N(*)C)c1]

wherein the left attachment point connects to —S(O)₂— and the right attachment point connects to LBM.

Embodiment 225. The compound of any one of embodiments 116-121 and 197, or a pharmaceutically acceptable salt thereof, wherein

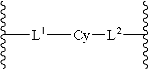

is

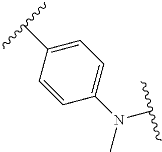 [SMILES: *c1ccc(N(*)C)cc1]

wherein the left attachment point connects to —S(O)₂— and the right attachment point connects to LBM.

Embodiment 226. The compound of any one of embodiments 116-121 and 197, or a pharmaceutically acceptable salt thereof, wherein

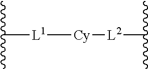

is

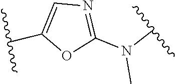 [SMILES: *c1cnc(N(*)C)o1]

wherein the left attachment point connects to —S(O)₂— and the right attachment point connects to LBM.

Embodiment 227. The compound of any one of embodiments 116-121 and 197, or a pharmaceutically acceptable salt thereof, wherein

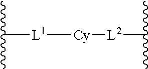

is

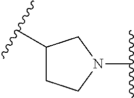 [SMILES: *C1CCN(*)C1]

wherein the left attachment point connects to —S(O)₂— and the right attachment point connects to LBM.

Embodiment 228. The compound of any one of embodiments 116-121 and 197, or a pharmaceutically acceptable salt thereof, wherein

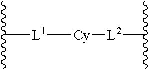

is

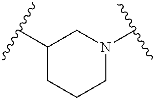 [SMILES: *C1CCCN(*)C1]

wherein the left attachment point connects to —S(O)₂— and the right attachment point connects to LBM.

Embodiment 229. The compound of any one of embodiments 116-121 and 197, or a pharmaceutically acceptable salt thereof, wherein

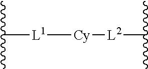

is

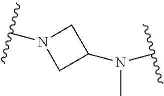 [SMILES: *N1CC(N(*)C)C1]

wherein the left attachment point connects to —S(O)₂— and the right attachment point connects to LBM.

Embodiment 230. The compound of any one of embodiments 116-121 and 197, or a pharmaceutically acceptable salt thereof, wherein

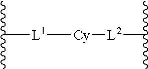

is

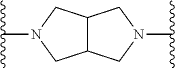 [SMILES: *N1CC2CN(*)CC2C1]

wherein the left attachment point connects to —S(O)₂— and the right attachment point connects to LBM.

Embodiment 231. The compound of any one of embodiments 116-121 and 197, or a pharmaceutically acceptable salt thereof, wherein

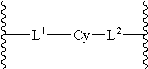

is

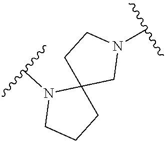 [SMILES: *N1CCC2(CCCN2*)C1]

wherein the left attachment point connects to —S(O)₂— and the right attachment point connects to LBM.

Embodiment 232. The compound of any one of embodiments 116-121 and 197, or a pharmaceutically acceptable salt thereof, wherein

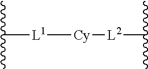

is

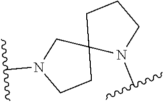 [SMILES: *N1CCC2(CCCN2*)C1]

wherein the left attachment point connects to —S(O)₂— and the right attachment point connects to LBM.

Embodiment 233. The compound of any one of embodiments 116-121 and 197, or a pharmaceutically acceptable salt thereof, wherein

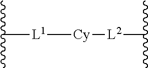

is

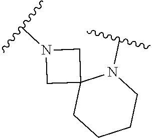 [SMILES: *N1CC2(CCCCN2*)C1]

wherein the left attachment point connects to —S(O)₂— and the right attachment point connects to LBM.

Embodiment 234. The compound of any one of embodiments 116-121 and 197, or a pharmaceutically acceptable salt thereof, wherein

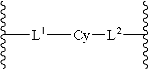

is

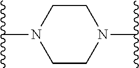 [SMILES: *N1CCN(*)CC1]

wherein the left attachment point connects to —S(O)₂— and the right attachment point connects to LBM.

Embodiment 235. The compound of any one of embodiments 116-121 and 197, or a pharmaceutically acceptable salt thereof, wherein

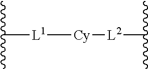

is

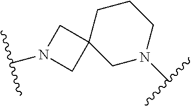 [SMILES: *N1CCCC2(C1)CN(*)C2]

wherein the left attachment point connects to —S(O)₂— and the right attachment point connects to LBM.

Embodiment 236. The compound of any one of embodiments 116-121 and 197, or a pharmaceutically acceptable salt thereof, wherein

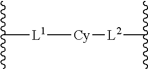

is

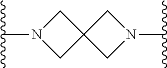 [SMILES: *N1CC2(C1)CN(*)C2]

wherein the left attachment point connects to —S(O)₂— and the right attachment point connects to LBM.

Embodiment 237. The compound of any one of embodiments 116-121 and 197, or a pharmaceutically acceptable salt thereof, wherein

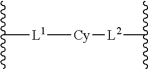

is

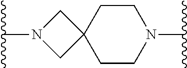 [SMILES: *N1CCC2(CC1)CN(*)C2]

wherein the left attachment point connects to —S(O)₂— and the right attachment point connects to LBM.

Embodiment 238. The compound of any one of embodiments 116-121 and 197, or a pharmaceutically acceptable salt thereof, wherein

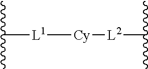

is

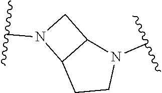 [SMILES: *N1CCC2C1CN2*]

wherein the left attachment point connects to —S(O)₂— and the right attachment point connects to LBM.

Embodiment 239. The compound of any one of embodiments 116-121 and 197, or a pharmaceutically acceptable salt thereof, wherein

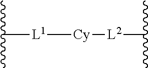

is

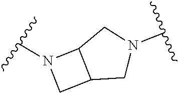 [SMILES: *N1CC2CN(*)C2C1]

wherein the left attachment point connects to —S(O)₂— and the right attachment point connects to LBM.

Embodiment 240. The compound of any one of embodiments 116-121 and 197, or a pharmaceutically acceptable salt thereof, wherein

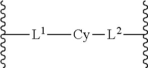

is

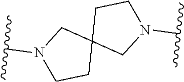 [SMILES: *N1CCC2(CCN(*)C2)C1]

wherein the left attachment point connects to —S(O)₂— and the right attachment point connects to LBM.

Embodiment 241. The compound of any one of embodiments 116-121 and 197, or a pharmaceutically acceptable salt thereof, wherein

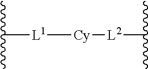

is

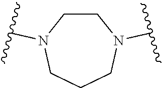 [SMILES: *N1CCCN(*)CC1]

wherein the left attachment point connects to —S(O)₂— and the right attachment point connects to LBM.

Embodiment 242. The compound of any one of embodiments 116-241, or a pharmaceutically acceptable salt thereof, wherein q is 0.

Embodiment 243. The compound of any one of embodiments 116-241, or a pharmaceutically acceptable salt thereof, wherein q is 1.

Embodiment 244. The compound of any one of embodiments 116-241, or a pharmaceutically acceptable salt thereof, wherein q is 2.

Embodiment 245. The compound of any one of embodiments 116-241, or a pharmaceutically acceptable salt thereof, wherein q is 3.

Embodiment 246. The compound of any one of embodiments 116-241, or a pharmaceutically acceptable salt thereof, wherein q is 4.

Embodiment 247. The compound of any one of embodiments 116-241, or a pharmaceutically acceptable salt thereof, wherein q is 5.

Embodiment 248. The compound of any one of embodiments 116-241, or a pharmaceutically acceptable salt thereof, wherein q is 6.

Embodiment 249. The compound of any one of embodiments 116-241, or a pharmaceutically acceptable salt thereof, wherein q is 7.

Embodiment 250. The compound of any one of embodiments 116-241, or a pharmaceutically acceptable salt thereof, wherein q is 8.

Embodiment 251. The compound of any one of embodiments 116-241, or a pharmaceutically acceptable salt thereof, wherein q is 9.

Embodiment 252. The compound of any one of embodiments 1-251, or a pharmaceutically acceptable salt thereof, wherein each R⁴ is independently selected from deuterium, halogen, —C₁₋₆ alkyl, —C₁₋₆ haloalkyl, —CN, —OR, —NR₂, —S(O)₂R, —S(O)₂NR₂, —S(O)R, —S(O)NR₂, —S(O)(NR)R, —C(O)NR₂, —C(O)N(R)OR, —OC(O)NR₂, —N(R)C(O)OR, —N(R)C(O)R, —N(R)C(O)NR₂, —N(R)S(O)₂NR₂ and —N(R)S(O)₂R, wherein R is H or C₁₋₆ alkyl.

Embodiment 253. The compound of any one of embodiments 1-251, or a pharmaceutically acceptable salt thereof, wherein each R⁴ is independently selected from -Me, -Et, —F, —Cl, —CF₃, —CN, —OH, —OMe, —NH₂, —NHMe and —NMe₂.

Embodiment 254. The compound of any one of embodiments 1-251, or a pharmaceutically acceptable salt thereof, wherein each R⁴ is independently selected from -Me and —F.

Embodiment 255. The compound of any one of embodiments 1-254, or a pharmaceutically acceptable salt thereof, wherein each R⁵ is independently selected from deuterium, C₁₋₆ aliphatic chain substituted with 0-3 instances of halo, halogen, —CN, —OR, —NR₂, —S(O)₂R, —S(O)₂NR₂, —S(O)R, —S(O)NR₂, —S(O)(NR)R, —C(O)NR₂, —C(O)N(R)OR, —OC(O)NR₂, —N(R)C(O)OR, —N(R)C(O)R, —N(R)C(O)NR₂, —N(R)S(O)₂NR₂ and —N(R)S(O)₂R, wherein R is H or C₁₋₆ alkyl.

Embodiment 256. The compound of any one of embodiments 1-254, or a pharmaceutically acceptable salt thereof, wherein each R⁵ is independently selected from -Me, -Et, —F, —Cl, —CF₃, —CN, —OH, —OMe, —NH₂, —NHMe and —NMe₂.

Embodiment 257. The compound of any one of embodiments 1-254, or a pharmaceutically acceptable salt thereof, wherein each R⁵ is independently selected from -Me and —F.

Embodiment 258. The compound of any one of embodiments 1-257, or a pharmaceutically acceptable salt thereof, wherein r is 0, 1 or 2.

Embodiment 259. The compound of any one of embodiments 1-257, or a pharmaceutically acceptable salt thereof, wherein r is 0.

Embodiment 260. The compound of any one of embodiments 1-257, or a pharmaceutically acceptable salt thereof, wherein r is 1.

Embodiment 261. The compound of any one of embodiments 1-257, or a pharmaceutically acceptable salt thereof, wherein r is 2.

Embodiment 262. The compound of any one of embodiments 1-257, or a pharmaceutically acceptable salt thereof, wherein s is 0, 1 or 2.

Embodiment 263. The compound of any one of embodiments 1-257, or a pharmaceutically acceptable salt thereof, wherein s is 0.

Embodiment 264. The compound of any one of embodiments 1-257, or a pharmaceutically acceptable salt thereof, wherein s is 1.

Embodiment 265. The compound of any one of embodiments 1-257, or a pharmaceutically acceptable salt thereof, wherein s is 2.

Embodiment 266. The compound of any one of embodiments 1-251, or a pharmaceutically acceptable salt thereof, wherein LBM is selected from

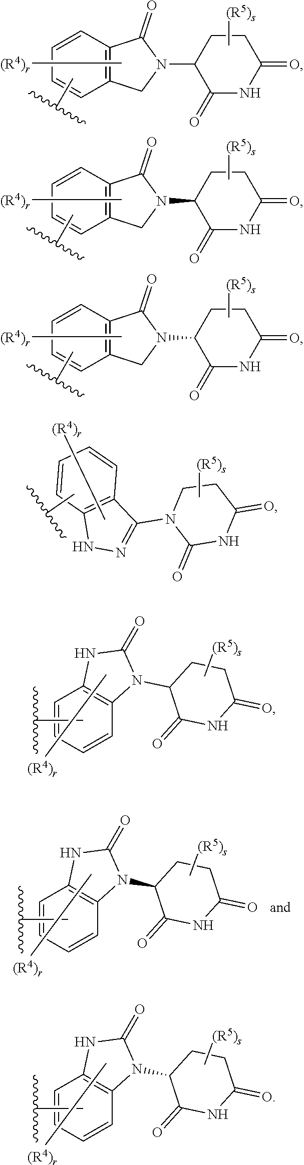

Embodiment 267. The compound of any one of embodiments 1-251, or a pharmaceutically acceptable salt thereof, wherein LBM is selected from

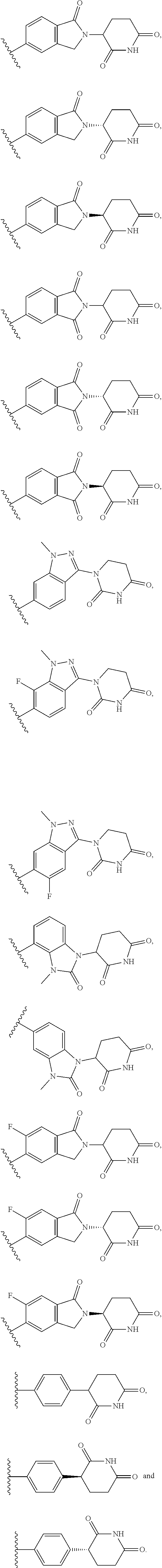

Embodiment 268. The compound of any one of embodiments 1-251, or a pharmaceutically acceptable salt thereof, wherein LBM is selected from

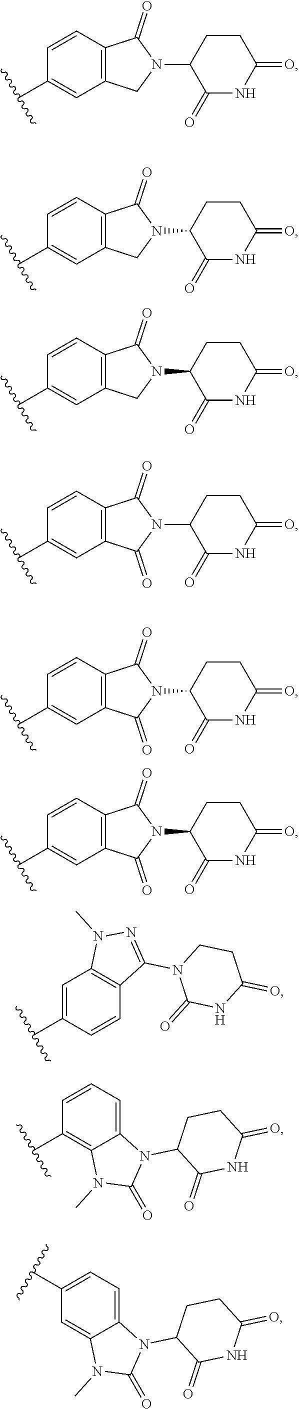

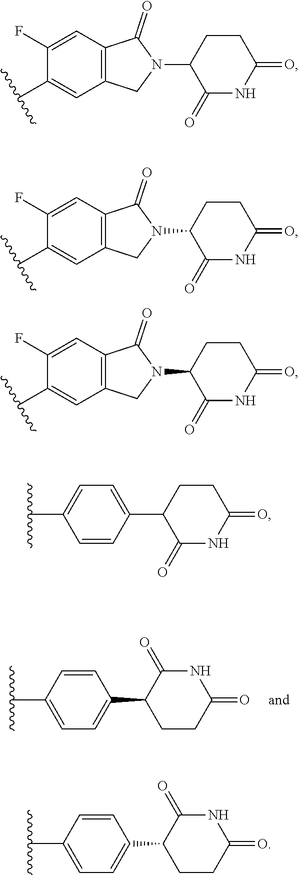

Embodiment 269. The compound of any one of embodiments 1-251, or a pharmaceutically acceptable salt thereof, wherein LBM is selected from

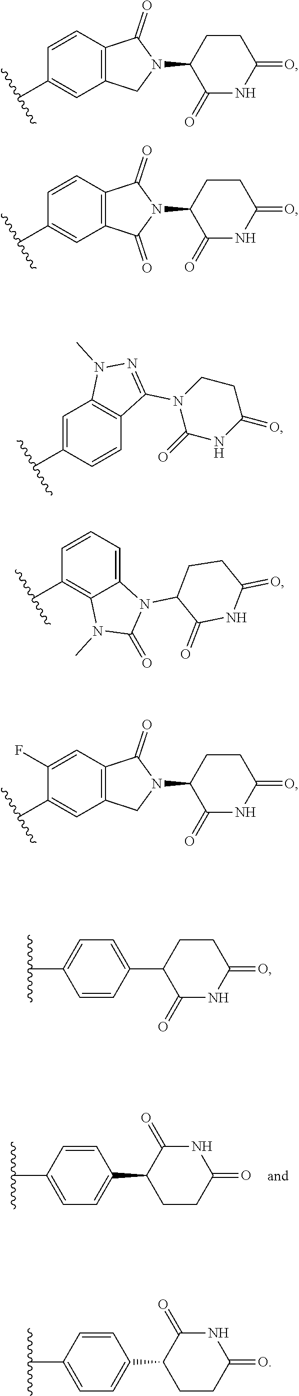

Embodiment 270. The compound of any one of embodiments 1-251, or a pharmaceutically acceptable salt thereof, wherein LBM is selected from

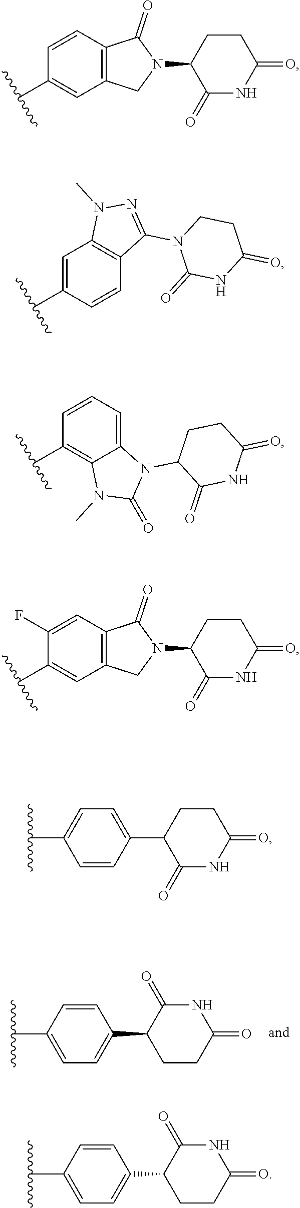

Embodiment 271. The compound of any one of embodiments 1-251, or a pharmaceutically acceptable salt thereof, wherein LBM is selected from

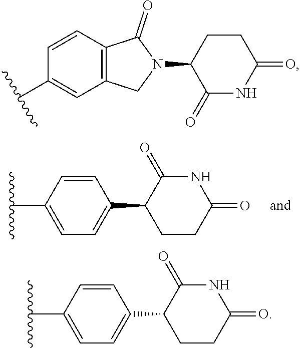

Embodiment 272. The compound of any one of embodiments 1-251, or a pharmaceutically acceptable salt thereof, wherein LBM is selected from

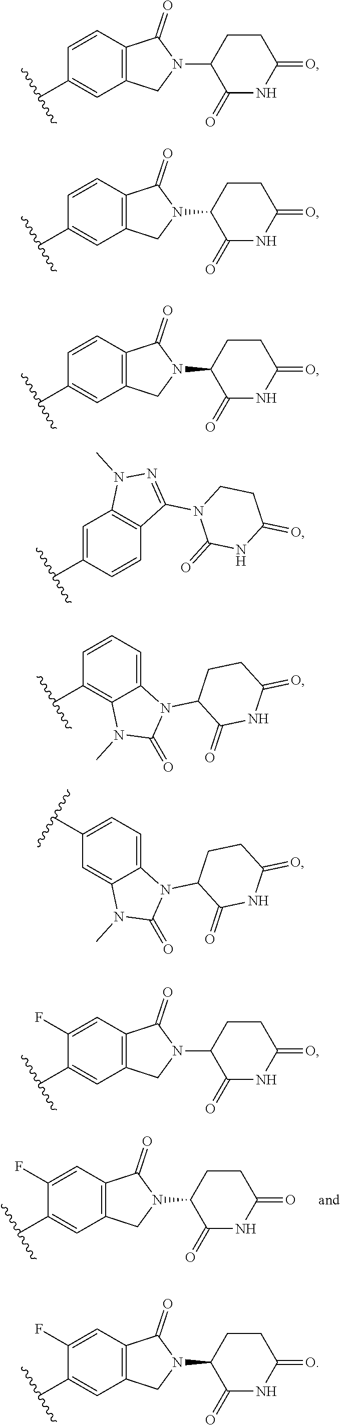

Embodiment 273. The compound of any one of embodiments 1-251, or a pharmaceutically acceptable salt thereof, wherein LBM is selected from

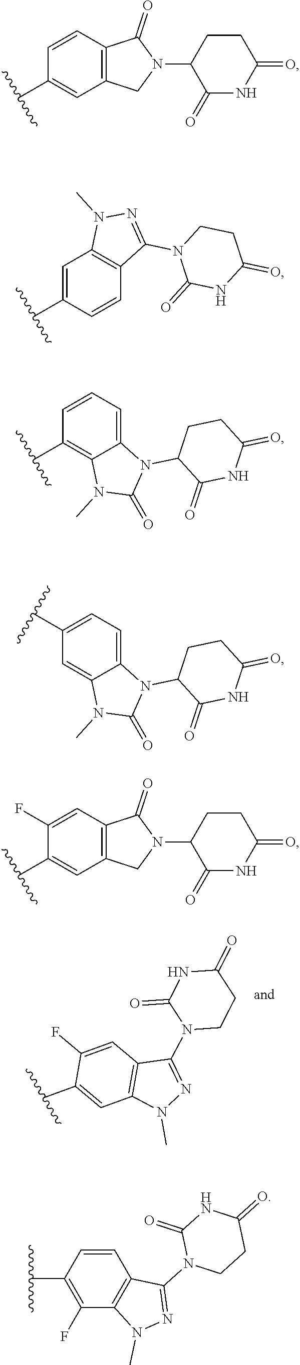

Embodiment 274. The compound of any one of embodiments 1-251, or a pharmaceutically acceptable salt thereof, wherein LBM is

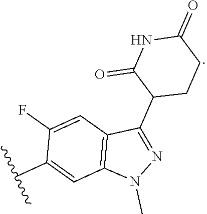 [SMILES: *c1cc2c(cc1F)c(C1CCC(=O)NC1=O)nn2C]

Embodiment 275. The compound of any one of embodiments 1-251, or a pharmaceutically acceptable salt thereof, wherein LBM is

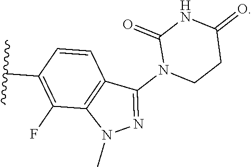 [SMILES: *c1ccc2c(N3CCC(=O)NC3=O)nn(C)c2c1F]

Embodiment 276. The compound of any one of embodiments 1-251, or a pharmaceutically acceptable salt thereof, wherein LBM is

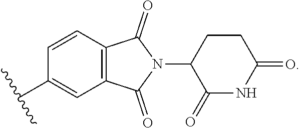 [SMILES: *c1ccc2c(c1)C(=O)N(C1CCC(=O)NC1=O)C2=O]

Embodiment 277. The compound of any one of embodiments 1-251, or a pharmaceutically acceptable salt thereof, wherein LBM is

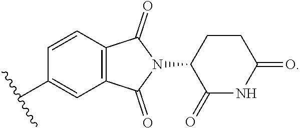 [SMILES: *c1ccc2c(c1)C(=O)N([C@@H]1CCC(=O)NC1=O)C2=O]

Embodiment 278. The compound of any one of embodiments 1-251, or a pharmaceutically acceptable salt thereof, wherein LBM is

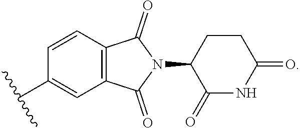 [SMILES: *c1ccc2c(c1)C(=O)N([C@H]1CCC(=O)NC1=O)C2=O]

Embodiment 279. The compound of any one of embodiments 1-251, or a pharmaceutically acceptable salt thereof, wherein LBM is

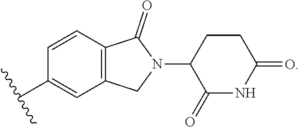 [SMILES: *c1ccc2c(c1)CN(C1CCC(=O)NC1=O)C2=O]

Embodiment 280. The compound of any one of embodiments 1-251, or a pharmaceutically acceptable salt thereof, wherein LBM is

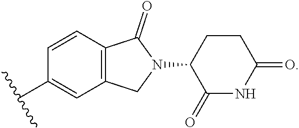 [SMILES: *c1ccc2c(c1)CN([C@@H]1CCC(=O)NC1=O)C2=O]

Embodiment 281. The compound of any one of embodiments 1-251, or a pharmaceutically acceptable salt thereof, wherein LBM is

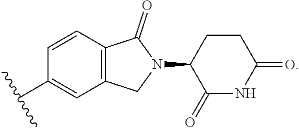 [SMILES: *c1ccc2c(c1)CN([C@H]1CCC(=O)NC1=O)C2=O]

Embodiment 282. The compound of any one of embodiments 1-251, or a pharmaceutically acceptable salt thereof, wherein LBM is

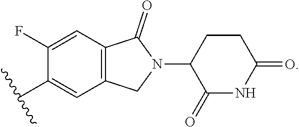 [SMILES: *c1cc2c(cc1F)C(=O)N(C1CCC(=O)NC1=O)C2]

Embodiment 283. The compound of any one of embodiments 1-251, or a pharmaceutically acceptable salt thereof, wherein LBM is

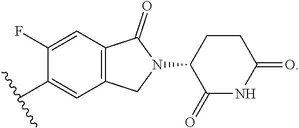 [SMILES: *c1cc2c(cc1F)C(=O)N([C@@H]1CCC(=O)NC1=O)C2]

Embodiment 284. The compound of any one of embodiments 1-251, or a pharmaceutically acceptable salt thereof, wherein LBM is

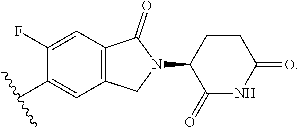 [SMILES: *c1cc2c(cc1F)C(=O)N([C@H]1CCC(=O)NC1=O)C2]

Embodiment 285. The compound of any one of embodiments 1-251, or a pharmaceutically acceptable salt thereof, wherein LBM is

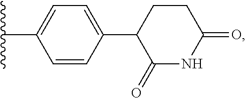 [SMILES: *c1ccc(C2CCC(=O)NC2=O)cc1]

Embodiment 286. The compound of any one of embodiments 1-251, or a pharmaceutically acceptable salt thereof, wherein LBM is

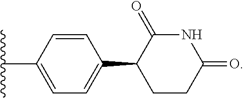 [SMILES: *c1ccc([C@@H]2CCC(=O)NC2=O)cc1]

Embodiment 287. The compound of any one of embodiments 1-251, or a pharmaceutically acceptable salt thereof, wherein LBM is

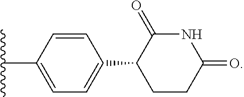 [SMILES: *c1ccc([C@H]2CCC(=O)NC2=O)cc1]

Embodiment 288. The compound of any one of embodiments 1-251, or a pharmaceutically acceptable salt thereof, wherein LBM is

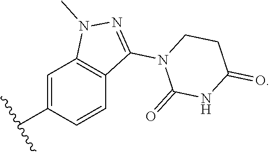 [SMILES: *c1ccc2c(N3CCC(=O)NC3=O)nn(C)c2c1]

Embodiment 289. The compound of any one of embodiments 1-251, or a pharmaceutically acceptable salt thereof, wherein LBM is

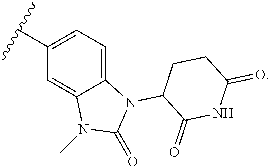 [SMILES: *c1ccc2c(c1)n(C)c(=O)n2C1CCC(=O)NC1=O]

Embodiment 290. The compound of any one of embodiments 1-251, or a pharmaceutically acceptable salt thereof, wherein LBM is

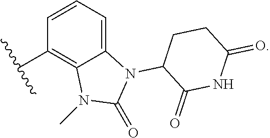 [SMILES: *c1cccc2c1n(C)c(=O)n2C1CCC(=O)NC1=O]

Embodiment 291. The compound of any one of embodiments 1-290, or a pharmaceutically acceptable salt thereof, wherein the compound is selected from the compounds of Table 1.

Embodiment 292. A pharmaceutical composition comprising a compound of any one of embodiments 1-291, or a pharmaceutically acceptable salt thereof, and a pharmaceutically acceptable carrier, adjuvant, or diluent.

Embodiment 293. A method of inhibiting CDK2 and/or CCNE (CCNE1 and/or CCNE2) signaling in a sample, e.g., in vivo or in vitro, by contacting CDK2 and/or CCNE (CCNE1 and/or CCNE2) with a compound of any one of embodiments 1-291, or a pharmaceutically acceptable salt thereof, or a composition of embodiment 292.

Embodiment 294. Use of a compound of any one of embodiments 1-291 or a composition of embodiment 292 in a method of inhibiting CDK2 and/or CCNE (CCNE1 and/or CCNE2) signaling in a sample, e.g., in vivo or in vitro, by contacting CDK2 and/or CCNE (CCNE1 and/or CCNE2) with the compound or composition.

Embodiment 295. Use of a compound of any one of embodiments 1-291 or a composition of embodiment 292 in the manufacturing of a medicament for inhibiting CDK2 and/or CCNE (CCNE1 and/or CCNE2) signaling in a sample, e.g., in vivo or in vitro.

Embodiment 296. A compound of any one of embodiments 1-291 or a composition of embodiment 292 for use in a method of inhibiting CDK2 and/or CCNE (CCNE1 and/or CCNE2) signaling in a sample, e.g., in vivo or in vitro, by contacting CDK2 and/or CCNE (CCNE1 and/or CCNE2) with the compound or composition.

Embodiment 297. A compound of any one of embodiments 1-291 or a composition of embodiment 292 for use in manufacturing of a medicament for inhibiting CDK2 and/or CCNE (CCNE1 and/or CCNE2) signaling in a sample, e.g., in vivo or in vitro, by contacting CDK2 and/or CCNE (CCNE1 and/or CCNE2) with the compound or composition.

Embodiment 298. The method, use, compound or composition for use of any one of embodiments 293-297, wherein the inhibiting of CDK2 and/or CCNE (CCNE1 and/or CCNE2) signaling comprises reducing the signaling activity of CDK2 and/or CCNE (CCNE1 and/or CCNE2) by at least 1%, 2%, 5%, 7.5%, 10%, 15%, 20%, 25%, 30%, 35%, 40%, 45%, 50%, 55%, 60%, 65%, 70%, 75%, 80%, 85%, 90%, 95%, 99%, e.g., relative to a reference standard.

Embodiment 299. The method, use, compound or composition for use of any one of embodiments 293-297, wherein the inhibiting of CDK2 and/or CCNE (CCNE1 and/or CCNE2) signaling comprises reducing the signaling activity of CDK2 and/or CCNE (CCNE1 and/or CCNE2) by at least 1-fold, 1.5-fold, 2-fold, 3-fold, 5-fold, 10-fold, 20-fold, 30-fold, 50-fold, 100-fold, or more, e.g., relative to a reference standard.

Embodiment 300. A method of inhibiting CDK2 signaling in a sample, e.g., in vivo or in vitro, by contacting CDK2 with a compound of any one of embodiments 1-291, or a pharmaceutically acceptable salt thereof, or a composition of embodiment 292.

Embodiment 301. Use of a compound of any one of embodiments 1-291, or a pharmaceutically acceptable salt thereof, or a composition of embodiment 292 in a method of inhibiting CDK2 signaling in a sample, e.g., in vivo or in vitro, by contacting CDK2 with a compound of any one of embodiments 1-291, or a pharmaceutically acceptable salt thereof, or a composition of embodiment 292.

Embodiment 302. Use of a compound of any one of embodiments 1-291, or a pharmaceutically acceptable salt thereof, or a composition of embodiment 292 in the manufacturing of a medicament for inhibiting CDK2 signaling in a sample, e.g., in vivo or in vitro.

Embodiment 303. A compound of any one of embodiments 1-291, or a pharmaceutically acceptable salt thereof, or a composition of embodiment 292 for use in a method of inhibiting CDK2 signaling in a sample, e.g., in vivo or in vitro, by contacting CDK2 with a compound of any one of embodiments 1-291, or a pharmaceutically acceptable salt thereof, or a composition of embodiment 292.

Embodiment 304. A compound of any one of embodiments 1-291, or a pharmaceutically acceptable salt thereof, or a composition of embodiment 292 for use in the manufacturing of a medicament for inhibiting CDK2 signaling in a sample, e.g., in vivo or in vitro.

Embodiment 305. The method, use, compound or composition for use of any one of embodiments 300-304, wherein the inhibiting of CDK2 signaling comprises reducing the signaling activity of CDK2 by at least 1%, 2%, 5%, 7.5%, 10%, 15%, 20%, 25%, 30%, 35%, 40%, 45%, 50%, 55%, 60%, 65%, 70%, 75%, 80%, 85%, 90%, 95%, 99%, e.g., relative to a reference standard.

Embodiment 306. The method, use, compound or composition for use of any one of embodiments 300-304, wherein the inhibiting of CDK2 signaling comprises reducing the signaling activity of CDK2 by at least 1-fold, 1.5-fold, 2-fold, 3-fold, 5-fold, 10-fold, 20-fold, 30-fold, 50-fold, 100-fold, or more, e.g., relative to a reference standard.

Embodiment 307. A method of inhibiting CCNE (CCNE1 and/or CCNE2) signaling in a sample, e.g., in vivo or in vitro, by contacting CCNE (CCNE1 and/or CCNE2) with a compound of any one of embodiments 1-291, or a pharmaceutically acceptable salt thereof, or a composition of embodiment 292.

Embodiment 308. Use of a compound of any one of embodiments 1-291, or a pharmaceutically acceptable salt thereof, or a composition of embodiment 292 in a method of inhibiting CCNE (CCNE1 and/or CCNE2) signaling in a sample, e.g., in vivo or in vitro, by contacting CCNE (CCNE1 and/or CCNE2) with a compound of any one of embodiments 1-291, or a pharmaceutically acceptable salt thereof, or a composition of embodiment 292.

Embodiment 309. Use of a compound of any one of embodiments 1-291, or a pharmaceutically acceptable salt thereof, or a composition of embodiment 292 in the manufacturing of a medicament for inhibiting CCNE (CCNE1 and/or CCNE2) signaling in a sample, e.g., in vivo or in vitro.

Embodiment 310. A compound of any one of embodiments 1-291, or a pharmaceutically acceptable salt thereof, or a composition of embodiment 292 for use in a method of inhibiting CCNE (CCNE1 and/or CCNE2) signaling in a sample, e.g., in vivo or in vitro, by contacting CCNE (CCNE1 and/or CCNE2) with a compound of any one of embodiments 1-291, or a pharmaceutically acceptable salt thereof, or a composition of embodiment 292.

Embodiment 311. A compound of any one of embodiments 1-291, or a pharmaceutically acceptable salt thereof, or a composition of embodiment 292 for use in the manufacturing of a medicament for inhibiting CCNE (CCNE1 and/or CCNE2) signaling in a sample, e.g., in vivo or in vitro.

Embodiment 312. The method, use, compound or composition for use of any one of embodiments 307-311, wherein the inhibiting of CCNE (CCNE1 and/or CCNE2) signaling comprises reducing the signaling activity of CCNE (CCNE1 and/or CCNE2) by at least 1%, 2%, 5%, 7.5%, 10%, 15%, 20%, 25%, 30%, 35%, 40%, 45%, 50%, 55%, 60%, 65%, 70%, 75%, 80%, 85%, 90%, 95%, 99%, e.g., relative to a reference standard.

Embodiment 313. The method, use, compound or composition for use of any one of embodiments 307-311, wherein the inhibiting CCNE (CCNE1 and/or CCNE2) signaling comprises reducing the signaling activity of CCNE (CCNE1 and/or CCNE2) by at least 1-fold, 1.5-fold, 2-fold, 3-fold, 5-fold, 10-fold, 20-fold, 30-fold, 50-fold, 100-fold, or more, e.g., relative to a reference standard.

Embodiment 314. A method of inhibiting CDK2 and CCNE (CCNE1 and/or CCNE2) signaling in a sample, e.g., in vivo or in vitro, by contacting CDK2 and/or CCNE (CCNE1 and/or CCNE2) with a compound of any one of embodiments 1-291, or a pharmaceutically acceptable salt thereof, or a composition of embodiment 292.

Embodiment 315. Use of a compound of any one of embodiments 1-291, or a pharmaceutically acceptable salt thereof, or a composition of embodiment 292 in a method of inhibiting CDK2 and CCNE (CCNE1 and/or CCNE2) signaling in a sample, e.g., in vivo or in vitro, by contacting CDK2 and/or CCNE (CCNE1 and/or CCNE2) with a compound of any one of embodiments 1-291, or a pharmaceutically acceptable salt thereof, or a composition of embodiment 292.

Embodiment 316. Use of a compound of any one of embodiments 1-291, or a pharmaceutically acceptable salt thereof, or a composition of embodiment 292 in the manufacturing of a medicament for inhibiting CDK2 and CCNE (CCNE1 and/or CCNE2) signaling in a sample, e.g., in vivo or in vitro.

Embodiment 317. A compound of any one of embodiments 1-291, or a pharmaceutically acceptable salt thereof, or a composition of embodiment 292 for use in a method of inhibiting CDK2 and CCNE (CCNE1 and/or CCNE2) signaling in a sample, e.g., in vivo or in vitro, by contacting CDK2 and/or CCNE (CCNE1 and/or CCNE2) with a compound of any one of embodiments 1-291, or a pharmaceutically acceptable salt thereof, or a composition of embodiment 292.

Embodiment 318. A compound of any one of embodiments 1-291, or a pharmaceutically acceptable salt thereof, or a composition of embodiment 292 for use in the manufacturing of a medicament for inhibiting CDK2 and CCNE (CCNE1 and/or CCNE2) signaling in a sample, e.g., in vivo or in vitro.

Embodiment 319. The method, use, compound or composition for use of any one of embodiments 314-318, wherein the inhibiting of CDK2 and CCNE (CCNE1 and/or CCNE2) signaling comprises reducing the signaling activity of CDK2 and/or CCNE (CCNE1 and/or CCNE2) by at least 1%, 2%, 5%, 7.5%, 10%, 15%, 20%, 25%, 30%, 35%, 40%, 45%, 50%, 55%, 60%, 65%, 70%, 75%, 80%, 85%, 90%, 95%, 99%, e.g., relative to a reference standard.

Embodiment 320. The method, use, compound or composition for use of any one of embodiments 314-318, wherein the inhibiting of CDK2 and CCNE (CCNE1 and/or CCNE2) signaling comprises reducing the signaling activity of CDK2 and/or CCNE (CCNE1 and/or CCNE2) by at least 1-fold, 1.5-fold, 2-fold, 3-fold, 5-fold, 10-fold, 20-fold, 30-fold, 50-fold, 100-fold, or more, e.g., relative to a reference standard.

Embodiment 321. A method of treating a CDK2 and/or CCNE (CCNE1 and/or CCNE2)-mediated disorder in a patient in need thereof, comprising administering to the patient a compound of any one of any one of embodiments 1-291, or a pharmaceutically acceptable salt thereof, or a composition of embodiment 292.

Embodiment 322. Use of a compound of any one of any one of embodiments 1-291, or a pharmaceutically acceptable salt thereof, or a composition of embodiment 292 in a method of treating a CDK2 and/or CCNE (CCNE1 and/or CCNE2)-mediated disorder in a patient in need thereof, comprising administering to the patient a compound of any one of any one of embodiments 1-291, or a pharmaceutically acceptable salt thereof, or a composition of embodiment 292.

Embodiment 323. Use of a compound of any one of any one of embodiments 1-291, or a pharmaceutically acceptable salt thereof, or a composition of embodiment 292 in the manufacturing of a medicament for treating a CDK2 and/or CCNE (CCNE1 and/or CCNE2)-mediated disorder in a patient in need thereof.

Embodiment 324. A compound of any one of any one of embodiments 1-291, or a pharmaceutically acceptable salt thereof, or a composition of embodiment 292 for use in a method of treating a CDK2 and/or CCNE (CCNE1 and/or CCNE2)-mediated disorder in a patient in need thereof, comprising administering to the patient a compound of any one of any one of embodiments 1-291, or a pharmaceutically acceptable salt thereof, or a composition of embodiment 292.

Embodiment 325. A compound of any one of any one of embodiments 1-291, or a pharmaceutically acceptable salt thereof, or a composition of embodiment 292 for use in the manufacturing of a medicament for treating a CDK2 and/or CCNE (CCNE1 and/or CCNE2)-mediated disorder in a patient in need thereof.

Embodiment 326. The method, use, compound or composition for use of any one of embodiments 321-325, wherein the CDK2 and/or CCNE (CCNE1 and/or CCNE2)-mediated disorder is cancer.

Embodiment 327. A method of treating a CDK2-mediated disorder in a patient in need thereof, comprising administering to the patient a compound of any one of any one of embodiments 1-291, or a pharmaceutically acceptable salt thereof, or a composition of embodiment 292.

Embodiment 328. Use of a compound of any one of any one of embodiments 1-291, or a pharmaceutically acceptable salt thereof, or a composition of embodiment 292 in a method of treating a CDK2-mediated disorder in a patient in need thereof, comprising administering to the patient a compound of any one of any one of embodiments 1-291, or a pharmaceutically acceptable salt thereof, or a composition of embodiment 292.

Embodiment 329. Use of a compound of any one of any one of embodiments 1-291, or a pharmaceutically acceptable salt thereof, or a composition of embodiment 292 in the manufacturing of a medicament for treating a CDK2-mediated disorder in a patient in need thereof.

Embodiment 330. A compound of any one of any one of embodiments 1-291, or a pharmaceutically acceptable salt thereof, or a composition of embodiment 292 for use in a method of treating a CDK2-mediated disorder in a patient in need thereof, comprising administering to the patient a compound of any one of any one of embodiments 1-291, or a pharmaceutically acceptable salt thereof, or a composition of embodiment 292.

Embodiment 331. A compound of any one of any one of embodiments 1-291, or a pharmaceutically acceptable salt thereof, or a composition of embodiment 292 for use in the manufacturing of a medicament for treating a CDK2-mediated disorder in a patient in need thereof.

Embodiment 332. The method, use, compound or composition for use of any one of embodiments 327-331, wherein the CDK2-mediated disorder is cancer.

Embodiment 333. A method of treating a CCNE (CCNE1 and/or CCNE2)-mediated disorder in a patient in need thereof, comprising administering to the patient a compound of any one of any one of embodiments 1-291, or a pharmaceutically acceptable salt thereof, or a composition of embodiment 292.

Embodiment 334. Use of a compound of any one of any one of embodiments 1-291, or a pharmaceutically acceptable salt thereof, or a composition of embodiment 292 in a method of treating a CCNE (CCNE1 and/or CCNE2)-mediated disorder in a patient in need thereof, comprising administering to the patient a compound of any one of any one of embodiments 1-291, or a pharmaceutically acceptable salt thereof, or a composition of embodiment 292.

Embodiment 335. Use of a compound of any one of any one of embodiments 1-291, or a pharmaceutically acceptable salt thereof, or a composition of embodiment 292 in the manufacturing of a medicament for treating a CCNE (CCNE1 and/or CCNE2)-mediated disorder in a patient in need thereof.

Embodiment 336. A compound of any one of any one of embodiments 1-291, or a pharmaceutically acceptable salt thereof, or a composition of embodiment 292 for use in a method of treating a CCNE (CCNE1 and/or CCNE2)-mediated disorder in a patient in need thereof, comprising administering to the patient a compound of any one of any one of embodiments 1-291, or a pharmaceutically acceptable salt thereof, or a composition of embodiment 292.

Embodiment 337. A compound of any one of any one of embodiments 1-291, or a pharmaceutically acceptable salt thereof, or a composition of embodiment 292 for use in the manufacturing of a medicament for treating a CCNE (CCNE1 and/or CCNE2)-mediated disorder in a patient in need thereof.

Embodiment 338. The method, use, compound or composition for use of any one of embodiments 333-337, wherein the CCNE (CCNE1 and/or CCNE2)-mediated disorder is cancer.

Embodiment 339. A method of treating a CDK2 and CCNE (CCNE1 and/or CCNE2)-mediated disorder in a patient in need thereof, comprising administering to the patient a compound of any one of any one of embodiments 1-291, or a pharmaceutically acceptable salt thereof, or a composition of embodiment 292.

Embodiment 340. Use of a compound of any one of any one of embodiments 1-291, or a pharmaceutically acceptable salt thereof, or a composition of embodiment 292 in a method of treating a CDK2 and CCNE (CCNE1 and/or CCNE2)-mediated disorder in a patient in need thereof, comprising administering to the patient a compound of any one of any one of embodiments 1-291, or a pharmaceutically acceptable salt thereof, or a composition of embodiment 292.

Embodiment 341. Use of a compound of any one of any one of embodiments 1-291, or a pharmaceutically acceptable salt thereof, or a composition of embodiment 292 in the manufacturing of a medicament for treating a CDK2 and CCNE (CCNE1 and/or CCNE2)-mediated disorder in a patient in need thereof.

Embodiment 342. A compound of any one of any one of embodiments 1-291, or a pharmaceutically acceptable salt thereof, or a composition of embodiment 292 for use in a method of treating a CDK2 and CCNE (CCNE1 and/or CCNE2)-mediated disorder in a patient in need thereof, comprising administering to the patient a compound of any one of any one of embodiments 1-291, or a pharmaceutically acceptable salt thereof, or a composition of embodiment 292.

Embodiment 343. A compound of any one of any one of embodiments 1-291, or a pharmaceutically acceptable salt thereof, or a composition of embodiment 292 for use in the manufacturing of a medicament for treating a CDK2 and CCNE (CCNE1 and/or CCNE2)-mediated disorder in a patient in need thereof.

Embodiment 344. The method, use, compound or composition for use of any one of embodiments 339-343, wherein the CDK2 and CCNE (CCNE1 and/or CCNE2)-mediated disorder is cancer.

Embodiment 345. The method, use, compound or composition for use of any one of embodiments 326, 332, 338 and 344, wherein the cancer is selected from ovarian cancer, gastric cancer, uterine cancer (e.g., endometrial cancer), and breast cancer (e.g., triple negative breast cancer (TNBC), hormone-receptor positive (HR+) breast cancer, HER2 positive (HER2+) positive breast cancer).

Embodiment 346. The method, use, compound or composition for use of any one of embodiments 326, 332, 338, 344 and 345, wherein the cancer is resistant to treatment with CDK 4/6 inhibitors.

### EXAMPLES

In order that the invention(s) described herein may be more fully understood, the following examples are set forth. The synthetic and biological examples described in this application are offered to illustrate the compounds, pharmaceutical compositions, and methods provided herein and are not to be construed in any way as limiting their scope. In the synthetic examples below, the descriptions of experimental procedures within a reaction sequence are listed in numerical order.

### Abbreviations

General

| | |
|---|---|
| anhy. | anhydrous |
| aq. | aqueous |
| satd. | saturated |
| min(s) | minute(s) |
| hr(s) | hour(s) |
| mL | milliliter |
| mmol | millimole(s) |
| mol | mole(s) |
| MS | mass spectrometry |
| NMR | nuclear magnetic resonance |
| TLC | thin layer chromatography |
| HPLC | high-performance liquid chromatography |
| Me | methyl |
| i-Pr | iso-propyl |
| t-Bu | tert-butyl |
| Ph | phenyl |
| Et | ethyl |
| Bz | benzoyl |

Spectrum

| | |
|---|---|
| Hz | hertz |
| δ | chemical shift |
| J | coupling constant |
| s | singlet |
| d | doublet |
| t | triplet |
| q | quartet |
| m | multiplet |
| br | broad |
| qd | quartet of doublets |
| dquin | doublet of quintets |
| dd | doublet of doublets |
| dt | doublet of triplets |

Solvents and Reagents

| | |
|---|---|
| (i-PrO)₄Ti | titanium tetraisopropoxide |
| 9-BBN | 9-borabicyclo[3.3.1]nonane |
| AcCl | acetyl chloride |
| ACN | Acetonitrile |
| AcOH | acetic acid |
| ADDP | 1,1′-(azodicarbonyl)dipiperidine |
| AlaOH | alanine |
| BHT | 2,6-di-t-butyl-4-methylphenoxide |
| BINAP | 2,2′-bis(diphenylphosphanyl)-1,1′-binaphthyl |
| Boc | t-butoxycarbonyl |
| BSA | Bovine Serum Albumin |
| Bu | butyl |
| BzCl | benzoyl chloride |
| CHCl₃ | chloroform |
| CsF | cesium fluoride |
| DAST | Diethylaminosulfurtrifluoride |
| DCC | dicyclohexylcarbodiimide |
| DCM | dichloromethane |
| DIAD | diisopropyl azodicarboxylate |
| DIPEA | N,N-diisopropylethylamine |
| DMAP | 4-(dimethylamino)pyridine |
| DMF | dimethylformamide |
| DMP | Dess-Martin periodinane |
| DMSO | dimethyl sulfoxide |
| dppf | 1,1′-bis(diphenylphosphino)ferrocene |
| DTT | DL-Dithiothreitol |
| Et₂O | diethyl ether |
| Et₃N | triethylamine |
| EtMgBr | ethylmagnesium bromide |
| EtOAc | ethyl acetate |
| EtOAc | ethyl acetate |
| EtOH | ethyl alcohol |
| H₂SO₄ | sulfuric acid |
| HCl | hydrochloric acid |
| i-PrMgCl | Isopropylmagnesium chloride |
| K₂CO₃ | potassium carbonate |
| KOH | potassium hydroxide |
| LAH | Lithium Aluminium Hydride |
| LDA | lithium diisopropylamide |
| LDH | Lactate Dehydrogenase |
| LiHMDS | lithium hexamethyldisilylamide |
| LiOH•H₂O | lithium hydroxide hydrates |
| MAD | methyl aluminum bis(2,6-di-t-butyl-4-methylphenoxide) |
| MeCN | acetonitrile |
| MeCN | acetonitrile |
| MeOH | methyl alcohol |
| MTBE | methyl tert-butyl ether |
| Na₂CO₃ | sodium carbonate |
| Na₂S₂O₃ | sodium thiosulfate |
| Na₂SO₄ | sodium sulfate |
| Na₂SO₄ | sodium sulfate |
| NaBH₄ | sodium borohydride |
| NaBH₄ | sodium borohydride |
| NADH | β-Nicotinamide adenine dinucleotide, reduced |
| NaHCO₃ | sodium bicarbonate |
| NaOH | sodium hydroxide |
| NBS | N-bromosuccinimide |
| NH₄Cl | ammonium chloride |
| PCC | pyridinium chlorochromate |
| Pd(t-Bu₃P)₂ | bis(tri-tert-butylphosphine)palladium(0) |
| PE | petroleum ether |
| PEP | Phospho(enol)pyruvic acid |
| Py | pyridine |
| RuPhos | 2-dicyclohexylphosphino-2′,6′-diisopropoxybiphenyl |
| TBAF | tetra-n-butylammonium fluoride |
| TBS | t-butyldimethylsilyl |
| TBSCl | tert-Butyl(chloro)dimethylsilane |
| t-BuOK | potassium tert-butoxide |
| ^{t}BuXPhos | 2-di-tert-butylphosphino-2′,4′,6′-triisopropylbiphenyl |
| TEA | triethylamine |
| TFA | trifluoroacetic acid |
| THF | tetrahydrofuran |
| Ti(OⁱPr)₄ | tetraisopropoxytitanium |
| TMS | trimethylsilyl |
| TMSCF₃ | (Trifluoromethyl)trimethylsilane |
| Ts | p-toluenesulfonyl |
| Xphos | Dicyclohexyl[2′,4′,6′-tris(propan-2- |
| | yl)[1,1′-biphenyl]-2-yl]phosphane |

### INTERMEDIATES

### 2-Methylcyclopentan-1-amine hydrochloride (Int-1)

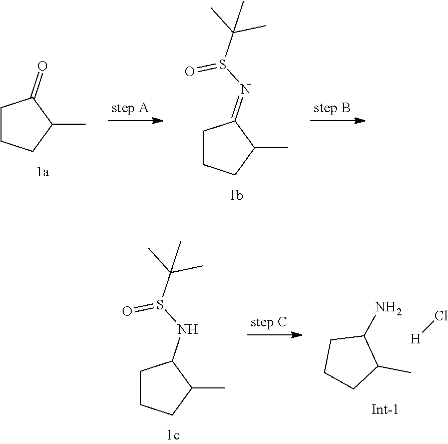

### Step A. 2-Methyl-N-[(1E)-2-methylcyclopentylidene]propane-2-sulfinamide (1b)

To a stirred solution of 1a (25 g, 254.725 mmol, 1 equiv) and tert-butanesulfinamide (37.05 g, 305.670 mmol, 1.2 equiv) in THF (250 mL) was added Ti(OEt)₄ (98.78 g, 433.032 mmol, 1.7 equiv) in portions at room temperature. The resulting mixture was stirred for 4 h at 55° C. The mixture was allowed to cool down to room temperature. The reaction was quenched with water (500 mL) at room temperature. The resulting mixture was filtered, the filter cake was washed with EtOAc (3×500 mL). The resulting filtrate was diluted with water (1000 mL) and extracted with EtOAc (3×500 mL). The combined organic layers were dried over anhydrous Na₂SO₄. After filtration, the filtrate was concentrated under reduced pressure. The residue was purified by silica gel column chromatography, eluted with PE/EtOAc (10:1) to afford 1b (37 g, 72.15%) as a yellow oil. LCMS (ESI, m/z): [M+H]⁺=202

### Step B. 2-Methyl-N-(2-methylcyclopentyl)propane-2-sulfinamide (1c)

To a stirred solution of 1b (37 g, 183.778 mmol, 1 equiv) in MeOH (370 mL) was added NaBH₄ (13.90 g, 367.556 mmol, 2 equiv) in portions at 0° C. The reaction mixture was stirred for 1 h at room temperature. Desired product could be detected by LCMS. The resulting mixture was diluted with water (500 mL) and extracted with EtOAc (3×500 mL). The combined organic layers were washed with brine (500 mL), dried over anhydrous C. After filtration, the filtrate was concentrated under reduced pressure. The resulting mixture was used in the next step directly without further purification. LCMS (ESI, m/z): [M+H]⁺=204

### Step C. 2-Methylcyclopentan-1-amine hydrochloride (Int-1)

A solution of 1c (37 g, 181.961 mmol, 1 equiv) and 4M HCl (gas) in 1,4-dioxane (227.45 mL, 909.805 mmol, 5 equiv) in dioxane (370 mL) was stirred for 2 h at room temperature. The resulting mixture was concentrated under reduced pressure. The precipitated solids were collected by filtration and washed with PE (3×100 mL). The resulting mixture Int-1 was used in the next step directly without further purification. LCMS (ESI, m/z): [M+H]⁺=100

### Tert-butyl 4-[7′-(2-methylcyclopentyl)-6′-oxospiro[cyclopropane-1,5′-pyrrolo[2,3-d]pyrimidin]-2′-ylamino] piperidine-1-carboxylate (Int-2)

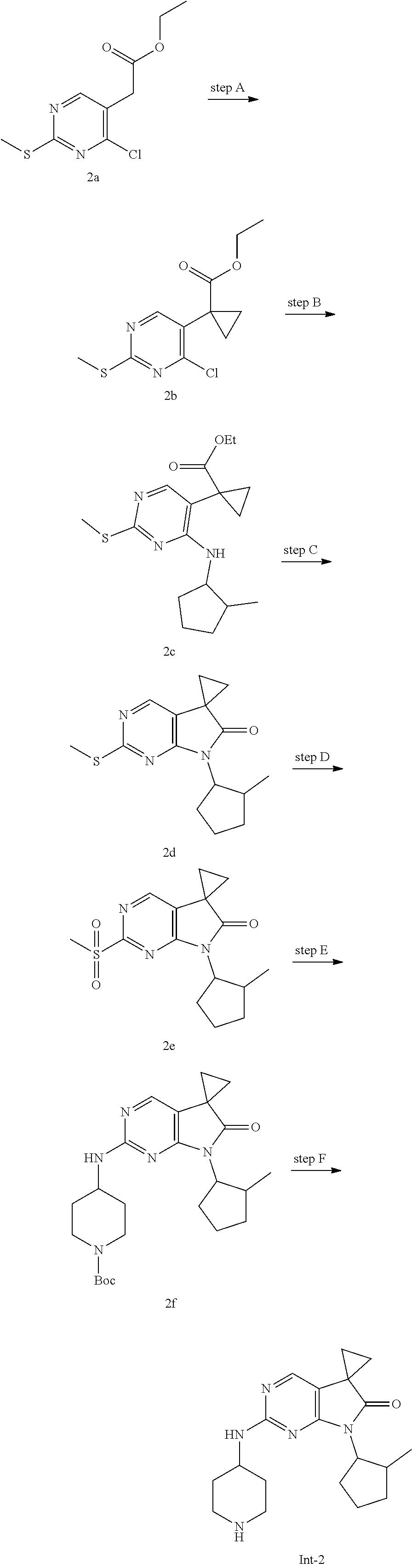

### Step A. Ethyl 1-(4-chloro-2-(methylthio)pyrimidin-5-yl)cyclopropane-1-carboxylate (2b)

A solution of 2a (25 g, 101.334 mmol, 1 equiv), dibromoethane (22.84 g, 121.601 mmol, 1.2 equiv) and NaH (6.08 g, 253.335 mmol, 2.5 equiv) in DMF (250 mL) was stirred for 2 h at room temperature under air atmosphere. The reaction was quenched with sat. aq. NH₄Cl (aq.) at 0° C. The aqueous layer was extracted with EtOAc (3×100 mL). The combined organic layers were washed with brine (100 mL), dried over anhydrous C. After filtration, the filtrate was concentrated under reduced pressure. The residue was purified by silica gel column chromatography, eluted with PE/EtOAc (5:1) to afford 2b (15.5 g, 53.28%) as a white oil. LCMS (ESI, m/z): [M+H]⁺=273

### Step B. Ethyl 1-{4-[(2-methylcyclopentyl)amino]-2-(methylsulfanyl)pyrimidin-5-yl}cyclopropane-1-carboxylate (2c)

To a stirred solution of 2b (8 g, 29.331 mmol, 1 equiv) and Int-1 (7.96 g, 58.662 mmol, 2 equiv) in DMSO (80 mL) was added DIEA (26.54 g, 205.317 mmol, 7 equiv) at room temperature. The resulting mixture was stirred for 14 h at 100° C. The mixture was allowed to cool down to room temperature. Desired product could be detected by LCMS. The resulting mixture was diluted with water (200 mL) and extracted with EtOAc (3×100 mL). The combined organic layers were washed with brine (100 mL), dried over anhydrous C. After filtration, the filtrate was concentrated under reduced pressure. The residue was purified by silica gel column chromatography, eluted with PE/EtOAc (1:1) to afford 2c (5.7 g, 57.93%) as a yellow oil. LCMS (ESI, m/z): [M+H]⁺=336

### Step C. 7′-(2-Methylcyclopentyl)-2′-(methylsulfanyl)spiro[cyclopropane-1,5′-pyrrolo[2,3-d]pyrimidin]-6′-one (2d)

To a solution of 2c (5.7 g, 16.991 mmol, 1 equiv) in THF (77 mL) was added sodium hydride (60% in oil, 1.31 g) at 0 degrees C. The mixture was stirred for 1 h. Desired product could be detected by LCMS. The reaction mixture was quenched by water. The resulting mixture was diluted with water (100 mL) and extracted with EtOAc (3×100 mL). The combined organic layers were washed with brine (100 mL), dried over anhydrous C. After filtration, the filtrate was concentrated under reduced pressure. The residue was purified by silica gel column chromatography, eluted with PE/EtOAc (3:1) to afford 2d (4.8 g, 97.62%) as a yellow solid. LCMS (ESI, m/z): [M+H]⁺=290

### Step D. 2′-Methanesulfonyl-7′-(2-methylcyclopentyl)spiro[cyclopropane-1,5′-pyrrolo[2,3-d]pyrimidin]-6′-one (2e)

A mixture of 2d (4.8 g, 16.586 mmol, 1 equiv) and oxone (8.37 g, 49.758 mmol, 3 equiv) in THF (40 mL) and H2O (40 mL) was stirred for 5 h at room temperature. Desired product could be detected by LCMS. The reaction was quenched with sat. sodium hyposulfite (aq.) at room temperature. The resulting mixture was diluted with water (100 mL) and extracted with EtOAc (3×100 mL). The combined organic layers were washed with brine (100 mL), dried over anhydrous C. After filtration, the filtrate was concentrated under reduced pressure. The residue was purified by silica gel column chromatography, eluted with PE/EtOAc (1:1) to afford 2e (4.2 g, 78.79%) as a white solid. LCMS (ESI, m/z): [M+H]⁺=322

### Step E. Tert-butyl 4-[7′-(2-methylcyclopentyl)-6′-oxospiro[cyclopropane-1,5′-pyrrolo[2,3-d]pyrimidin]-2′-ylamino]piperidine-1-carboxylate (2f)

To a solution of tert-butyl 4-aminopiperidine-1-carboxylate (2.62 g, 13.068 mmol, 1 equiv) in THF (420 mL) was added i-PrMgCl—LiCl (1.3 M in THF) (2.09 g, 14.375 mmol, 1.1 equiv) at 0 degrees C. The mixture was stirred for 30 min. 2e (4.2 g, 13.068 mmol, 1 equiv) was added and the mixture was allowed to warm to 50° C. and stirred for overnight. The reaction mixture was quenched by water and extracted with DCM (3*25 mL). The resulting mixture was concentrated under reduced pressure. The residue was purified by silica gel column chromatography, eluted with CH₂Cl₂/MeOH (30:1) to afford 2f (1.4 g, 24.26%) as a yellow solid. LCMS (ESI, m/z): [M+H]⁺=442

### Step F. Tert-butyl 4-[7′-(2-methylcyclopentyl)-6′-oxospiro[cyclopropane-1,5′-pyrrolo[2,3-d]pyrimidin]-2′-ylamino]piperidine-1-carboxylate (Int-2)

To a stirred mixture of 2f (450 mg, 1.019 mmol, 1 equiv) in ACN (9 mL) was added 4 M HCl (gas) in 1,4-dioxane (1.8 mL). The resulting mixture was stirred for 1 h at 0° C. The resulting mixture was concentrated under reduced pressure to afford Int-2 (380 mg, crude) as a white solid. The crude product was used in the next step directly without further purification. LCMS (ESI, m/z): [M+H]⁺=342.

### 3-(4-Bromo-3-methyl-2-oxo-1,3-benzodiazol-1-yl)piperidine-2,6-dione (Int-3)

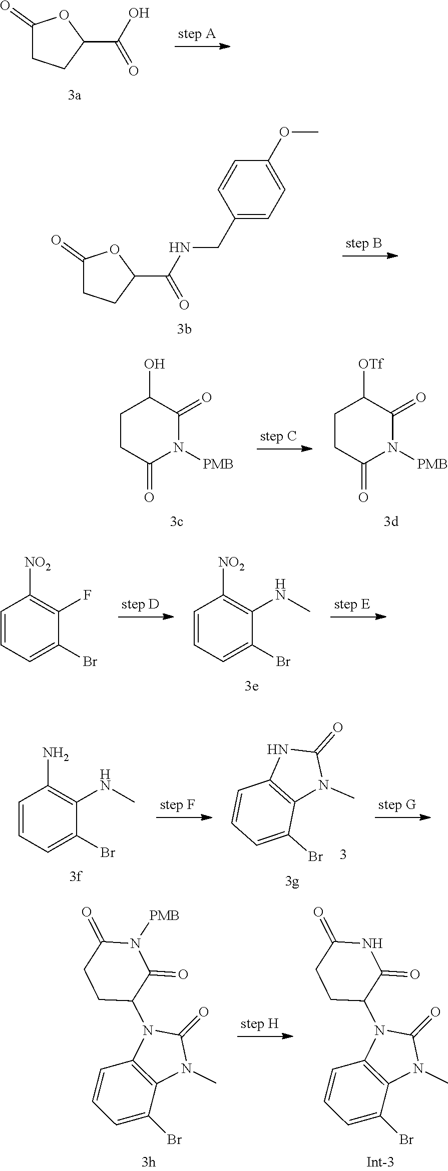

### Step A. N-[(4-Methoxyphenyl)methyl]-5-oxooxolane-2-carboxamide (3b)

To a solution of 3a (100 g, 768.645 mmol, 1 equiv) in DCM (1000 mL) was added benzenemethanamine, 4-methoxy- (105.44 g, 768.645 mmol, 1 equiv). Then DIEA (298.04 g, 2305.935 mmol, 3 equiv) was added and the reaction mixture was stirred at room temperature overnight. Then the mixture was concentrated and the residue was purified by column chromatography on silica gel (EtOAc:PE=1:1) to give the product 3b (100 g, 52.19%). LCMS (ESI, m/z): [M+H]⁺=250.

### Step B. 3-Hydroxy-1-[(4-methoxyphenyl)methyl]piperidine-2,6-dione (3c)

To a solution of 3b (100 g, 401.178 mmol, 1 equiv) in THF (1000 mL) was added t-BuOK (45.02 g, 401.178 mmol, 1 equiv). The resulting mixture was stirred for 1 h at −78° C. Then the mixture was concentrated and the residue was purified by column chromatography on silica gel (EtOAc:PE=1:1) to give the product 3c (55 g, 55.00%) as a yellow solid. LCMS (ESI, m/z): [M+H]⁺=250.

### Step C. 1-[(4-Methoxyphenyl)methyl]-2,6-dioxopiperidin-3-yl trifluoromethanesulfonate (3d)

To a solution of 3c (55 g, 220.648 mmol, 1 equiv) in DCM was added Pyridine (31.65 g, 400.127 mmol, 1.81 equiv). Then (TfO)₂O (93.37 g, 330.972 mmol, 1.5 equiv) was added and the reaction mixture was stirred at 0° C. 1 h. The resulting mixture was concentrated under vacuum and the residue was purified by column chromatography on silica gel (EtOAc:PE=1:3) to give the product 3d (50 g, 59.43%) as a yellow liquid. LCMS (ESI, m/z): [M+H]⁺=382.

### Step D. 2-Bromo-N-methyl-6-nitroaniline (3e)

To a stirred solution of 1-bromo-2-fluoro-3-nitrobenzene (50 g, 227.276 mmol, 1 equiv) and CH₃NH₂HCl (23.02 g, 340.914 mmol, 1.5 equiv) in acetonitrile was added K₂CO₃ (94.23 g, 681.828 mmol, 3 equiv) at 25° C. The resulting mixture was stirred for 3 h at 60° C. The resulting mixture was concentrated under reduced pressure. The product was precipitated by the addition of H₂O (500 ml). The precipitated solids were collected by filtration and washed with H2O (300 ml). This resulted in 2-bromo-N-methyl-6-nitroaniline (40 g, 76.17%) as a light yellow solid. LCMS (ESI, m/z): [M+H]⁺=231.

### Step E. 6-Bromo-N1-methylbenzene-1,2-diamine (3f)

To a stirred solution of 3e (40 g, 173.123 mmol, 1 equiv) in EtOAc (300 mL)/AcOH (100 mL)/H₂O (30 mL) was added Fe (38.67 g, 692.492 mmol, 4 equiv) in portions. The resulting mixture was stirred for 1 h at 60° C. The resulting mixture was filtered, the filter cake was washed with DCM (200 mL*3). The filtrate was concentrated under reduced pressure. The product was precipitated by the addition of H₂O (300 ml). The precipitated solids were collected by filtration and washed with H2O (200 mL). This resulted in 3f (30 g, 86.18%) as a light yellow solid. LCMS (ESI, m/z): [M+H]⁺=201.

### Step F. 7-Bromo-1-methyl-3H-1,3-benzodiazol-2-one (3g)

To a stirred solution of 3f (30 g, 149.204 mmol, 1 equiv) in acetonitrile (300 mL) was added CDI (36.29 g, 223.806 mmol, 1.5 equiv). The resulting mixture was stirred for 1 h at 80° C. The resulting mixture was concentrated under reduced pressure. The product was precipitated by the addition of H₂O (400 ml). The precipitated solids were collected by filtration and washed with H2O (300 mL). This resulted in 3g (30 g, 88.55%) as a pink solid. LCMS (ESI, m/z): [M+H]⁺=227.

### Step G. 3-(4-Bromo-3-methyl-2-oxo-1,3-benzodiazol-1-yl)-1-[(4-methoxyphenyl)methyl]piperidine-2,6-dione (3 h)

To a solution of 3g (23 g, 101.294 mmol, 1 equiv) in THF (598 mL) was added t-BuOK (13.8 g, 122.980 mmol, 1.21 equiv) at 0° C. The mixture was stirred at 0° C. for 30 min, then 3d (46 g, 120.634 mmol, 1.19 equiv) was added and the reaction mixture was stirred at 0° C. for 1 h. Then the mixture was concentrated and the residue was purified by column chromatography on silica gel (EtOAc:PE=1:1) to give the product 3 h (33 g, 71.08%) as a brown solid. LCMS (ESI, m/z): [M+H]⁺=458.

### Step H. 3-(4-Bromo-3-methyl-2-oxo-1,3-benzodiazol-1-yl)piperidine-2,6-dione (Int-3)

To a solution of 3 h (33 g, 72.003 mmol, 1 equiv) in toluene (600 mL) was added Methanesulfonic acid (210 mL). The mixture was stirred at 110° C. for 3 h. The resulting mixture was concentrated under vacuum. The product was precipitated by the addition of water. The precipitated solids were collected by filtration and washed with water. Then the mixture was concentrated and the residue was purified by column chromatography on silica gel (EtOAc:DCM=1:1) to give the product Int-3 (14.3 g, 58.73%) as a brown solid. LCMS (ESI, m/z): [M+H]⁺=338.

### 3-(5-Bromo-3-methyl-2-oxo-2,3-dihydro-1H-benzo[d]imidazol-1-yl)piperidine-2,6-dione (Int-4)

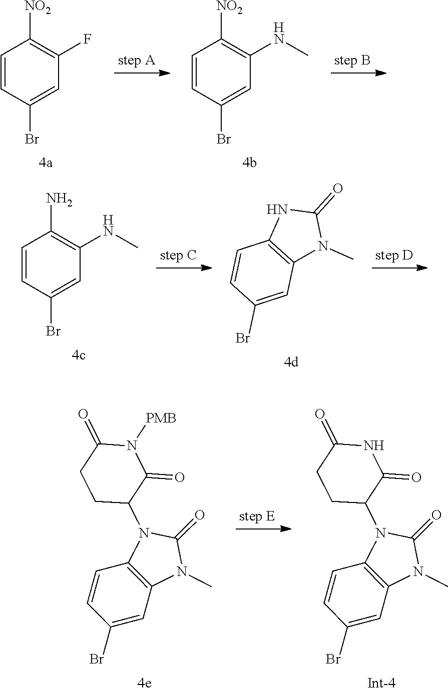

### Step A. 5-Bromo-N-methyl-2-nitroaniline (4b)

4a (250 g, 1.136 mmol, 1 equiv) was dissolved in ACN (2500 mL), MeNH₂·HCl (153 g, 2.272 mol, 2 equiv) and K₂CO₃ (470 g, 3.408 mol, 3 equiv) was added, the mixture was stirred at 60° C. 3 h. When LCMS showed the starting material was consumed, the solvent was evaporated and the resulting crude was dissolved in EtOAc (3000 mL) and washed with water (2000 mL×2) and brine (2000 mL), dried over anhydrous C. The solid was filtered and the filtrate was concentrated to give the crude product 4b as yellow solid (270 g, 90%). LCMS (ESI, m/z): [M+H]⁺=231

### Step B. 5-Bromo-N1-methylbenzene-1,2-diamine (4c)

4b (270 g, 1.163 mol, 1 equiv) was dissolved in NH₄C1 (187 g, 3.491 mol, 3 equiv), then EtOAc (2500 mL) and H₂O (500 mL were added. The mixture was warmed to 50° C., then Fe powder (92 g, 1.745 mol, 1.5 equiv) was added and the mixture was heated to 80° C. about 30 min. EtOAc (3000 mL) and H₂O (3000 mL) were added, the organic phase was washed with H₂O (5000 mL×2), the organic phase was dried over anhydrous C. The solid was filtered and the filtrate was concentrated to give the product 4c as yellow solid (240 g, yield 90%). LCMS (ESI, m/z): [M+H]⁺=201

### Step C. 6-Bromo-1-methyl-1H-benzo[d]imidazol-2(3H)-one (4d)

4c (210 g, 1.04 mol, 1 equiv) was dissolved in MeCN (2000 mL). CDI (252 g, 1.56 mol, 1.5 equiv) was added. The mixture was reflux about 2 hours under 80° C. When LCMS showed the staring material was consumed, the solvent was evaporated and the resulting crude was purified by column chromatography on silica gel (EtOAc:PE=1:1) to give the product 4d as brome solid (190 g, yield 85%). LCMS (ESI, m/z): [M+H]⁺=227

### Step D. 3-(5-Bromo-3-methyl-2-oxo-2,3-dihydro-1H-benzo[d]imidazol-1-yl)-1-(4-methoxybenzyl)piperi-dine-2,6-dione (4e)

To a solution of 4d (50 g, 0.22 mol, 1 equiv) in THF (500 mL) was added tert-butoxysodium (25 g, 0.22 mol, 1 equiv) at 0° C. The mixture was stirred at 0° C. for 30 min, then 3d (84 g, 0.22 mol, 1 equiv) was added and the reaction mixture was stirred at room temperature overnight. Then the mixture was concentrated and the residue was purified by column chromatography on silica gel (EtOAc:DCM=1:1) to give the product 4e (70 g, 37%) as brome solid. LCMS (ESI, m/z): [M+H]⁺=458

### Step E. 3-(5-Bromo-3-methyl-2-oxo-2,3-dihydro-1H-benzo[d]imidazol-1-yl)piperidine-2,6-dione (Int-4)

To a solution of 4e (70 g, 0.153 mol, 1 equiv) in toluene (1400 mL) was added Methanesulfonic acid (490 mL, 9.053 mol, 60 equiv). The reaction mixture was heated to 110° C. about 2 hours under N₂. The reaction mixture was cooled to room temperature then the solvent was removed by reduced pressure. The residue was purified by trituration with water (2000 mL). The solid was purified by column chromatography on silica gel (EtOAc:DCM=1:1) to give the product Int-4 (37 g, 72%). LCMS (ESI, m/z): [M+H]⁺=

### 2′-Chloro-7′-[(1R,3R)-3-(oxan-2-yloxy)cyclohexyl]spiro[cyclopropane-1,5′-pyrrolo[2,3-d]pyrimidin]-6′-one (Int-5)

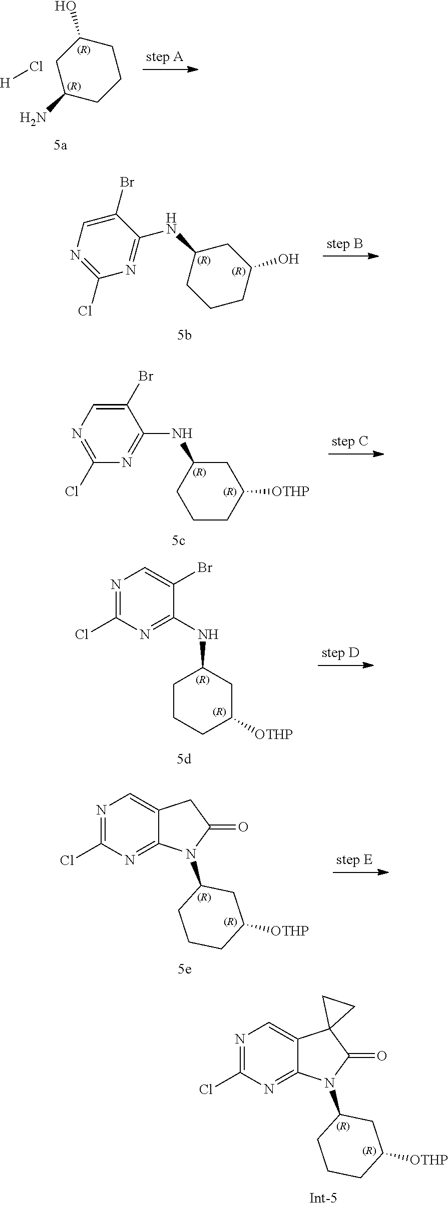

### Step A. (1R,3R)-3-((5-Bromo-2-chloropyrimidin-4-yl)amino)cyclohexan-1-ol (5b)

To a stirred solution of 5a (4 g, 34.729 mmol, 1 equiv) in THF (40 mL) were added 5-bromo-2,4-dichloropyrimidine (15.83 g, 69.458 mmol, 2 equiv) and DIEA (13.47 g, 104.187 mmol, 3 equiv). The resulting mixture was stirred for 15 hours at 25° C. under nitrogen atmosphere. Desired product could be detected by LCMS. The resulting mixture was diluted with saturated aqueous NH₄C1 (20 mL). The resulting mixture was extracted with EtOAc (2×50 mL). The combined organic layers were washed with brine (15 mL), dried over anhydrous sodium sulfate. After filtration, the filtrate was concentrated under reduced pressure. The residue was purified by silica gel column chromatography, eluted with 0% to 50% EtOAc in petroleum ether. Pure fractions were evaporated to dryness to afford 5b (8 g, 67.62%) as an off-white oil. LCMS (ESI, m/z): [M+H]⁺ 306.0.

### Step B. 5-Bromo-2-chloro-N-[(1R,3R)-3-(oxan-2-yloxy)cyclohexyl]pyrimidin-4-amine (5c)

To a stirred solution of 5b (10 g, 32.617 mmol, 1 equiv) in CH₂Cl₂ (100 mL) were added DHP (5.49 g, 65.234 mmol, 2 equiv)) and PPTS (0.82 g, 3.262 mmol, 0.1 equiv). The resulting mixture was stirred for 1 hour at 25° C. under nitrogen atmosphere. Desired product could be detected by LCMS. The resulting mixture was diluted with saturated aqueous NH₄C1 (50 mL). The resulting mixture was extracted with EtOAc (2×100 mL). The combined organic layers were washed with brine (50 mL), dried over anhydrous sodium sulfate. After filtration, the filtrate was concentrated under reduced pressure. The residue was purified by silica gel column chromatography, eluted with 0% to 15% EtOAc in petroleum ether. Pure fractions were evaporated to dryness to afford 5c (12 g, 89.46%) as a yellow oil. LCMS (ESI, m/z): [M+H]⁺ 390.1.

### Step C. Tert-butyl 2-(2-chloro-4-{[(1R,3R)-3-(oxan-2-yloxy)cyclohexyl]amino}pyrimidin-5-yl)acetate (5d)

To a stirred solution of 5c (14 g, 35.832 mmol, 1 equiv) in dioxane (140 mL) were added QPhos (2.54 g, 3.583 mmol, 0.1 equiv) and Pd₂(dba)₃ (2.06 g, 3.583 mmol, 0.1 equiv) under nitrogen atmosphere. The resulting mixture was stirred for 1 hour at 50° C. under nitrogen atmosphere. Desired product could be detected by LCMS. The resulting mixture was diluted with saturated aqueous NH₄C1 (50 mL). The resulting mixture was extracted with EtOAc (2×100 mL). The combined organic layers were washed with brine (30 mL), dried over anhydrous sodium sulfate. After filtration, the filtrate was concentrated under reduced pressure. The residue was purified by silica gel column chromatography, eluted with 0% to 25% EtOAc in petroleum ether. Pure fractions were evaporated to dryness to afford 5d (7 g, 43.57%) as a yellow oil. LCMS (ESI, m/z): [M+H]⁺ 426.2.

### Step D. 2-Chloro-7-[(1R,3R)-3-(oxan-2-yloxy)cyclohexyl]-5H-pyrrolo[2,3-d]pyrimidin-6-one (5e)

To a stirred solution of 5d (1 g, 2.348 mmol, 1 equiv) in THF (10 mL) was added NaH (0.23 g, 9.392 mmol, 4 equiv). The resulting mixture was stirred for 0.5 hours at 25° C. under nitrogen atmosphere. Desired product could be detected by LCMS. The resulting mixture was diluted with saturated aqueous NH₄C1 (20 mL). The resulting mixture was extracted with EtOAc (2×50 mL). The combined organic layers were washed with brine (20 mL), dried over anhydrous sodium sulfate. After filtration, the filtrate was concentrated under reduced pressure. The residue was purified by silica gel column chromatography, eluted with 0% to 15% EtOAc in petroleum ether. Pure fractions were evaporated to dryness to afford 5e (400 mg, 43.58%) as a yellow oil. LCMS (ESI, m/z): [M+H]⁺ 352.1.

### Step E. 2′-Chloro-7′-[(1R,3R)-3-(oxan-2-yloxy)cyclohexyl]spiro[cyclopropane-1,5′-pyrrolo[2,3-d]pyrimidin]-6′-one (Int-5)

To a stirred solution of 5e (4 g, 11.369 mmol, 1 equiv) in DMF (40 mL) were added DBU (5.19 g, 34.107 mmol, 3 equiv) and diphenylvinylsulfonium triflate (5.36 g, 14.780 mmol, 1.3 equiv). The resulting mixture was stirred for 1 hour at 25° C. under nitrogen atmosphere. Desired product could be detected by LCMS. The resulting mixture was diluted with saturated aqueous NH₄C1 (20 mL). The resulting mixture was extracted with EtOAc (2×30 mL). The combined organic layers were washed with brine (2×10 mL), dried over anhydrous sodium sulfate. After filtration, the filtrate was concentrated under reduced pressure. The residue was purified by silica gel column chromatography, eluted with 0% to 15% EtOAc in petroleum ether. Pure fractions were evaporated to dryness to afford Int-5 (4 g, 83.80%) as a yellow oil. LCMS (ESI, m/z): [M+H]⁺ 378.1.

### 2′-((1-((3-(bromomethyl)phenyl)sulfonyl)piperidin-4-yl)amino)-7′-((1R,3R)-3-hydroxycyclohexyl)spiro [cyclopropane-1,5′-pyrrolo[2,3-d]pyrimidin]-6′(7′H)-one (Int-6) & 2′-((1-((3-(bromomethyl)phenyl) sulfonyl)piperidin-4-yl)amino)-7′-((1R,3R)-3-((tetrahydro-2H-pyran-2-yl)oxy)cyclohexyl)spiro [cyclopropane-1,5′-pyrrolo[2,3-d]pyrimidin]-6′(7′H)-one (Int-7)

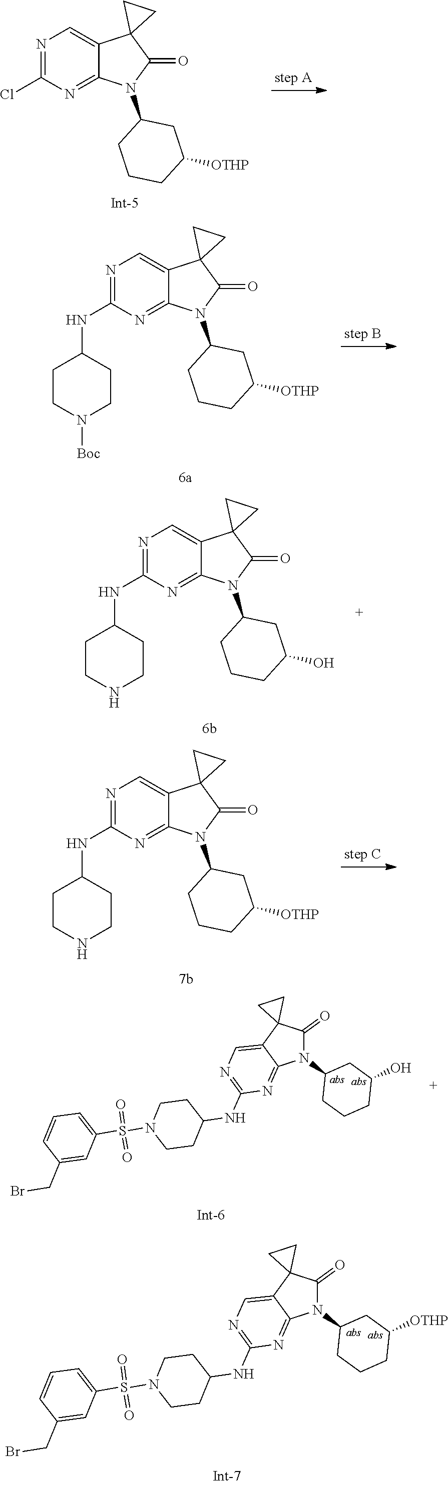

### Step A. Tert-butyl 4-{7′-[(1R,3R)-3-(oxan-2-yloxy)cyclohexyl]-6′-oxospiro[cyclopropane-1,5′-pyrrolo[2,3-d]pyrimidin]-2′-ylamino}piperidine-1-carboxylate (6a)

To a stirred solution of Int-5 (1.5 g, 3.970 mmol, 1 equiv) in dioxane (15 mL) were added tert-butyl 4-aminopiperidine-1-carboxylate (1.59 g, 7.940 mmol, 2 equiv), Pd₂(dba)₃ (0.23 g, 0.397 mmol, 0.1 equiv), RuPhos (0.19 g, 0.397 mmol, 0.1 equiv) and Cs₂CO₃ (3.88 g, 11.910 mmol, 3 equiv) under nitrogen atmosphere. The resulting mixture was stirred for 12 hours at 90° C. under nitrogen atmosphere. Desired product could be detected by LCMS. The resulting mixture was diluted with saturated aqueous NH₄C1 (20 mL). The resulting mixture was extracted with EtOAc (2×50 mL). The combined organic layers were washed with brine (20 mL), dried over anhydrous sodium sulfate. After filtration, the filtrate was concentrated under reduced pressure. The residue was purified by silica gel column chromatography, eluted with 0% to 60% EtOAc in petroleum ether. Pure fractions were evaporated to dryness to afford 6a (1000 mg, 44.18%) as a yellow solid. LCMS (ESI, m/z): [M+H]⁺ 542.3.

### Step B. 7′-((1R,3R)-3-Hydroxycyclohexyl)-2′-(piperidin-4-ylamino)spiro[cyclopropane-1,5′-pyrrolo [2,3-d]pyrimidin]-6′(7′H)-one (6b) & 2′-(Piperidin-4-ylamino)-7′-((1R,3R)-3-((tetrahydro-2H-pyran-2-yl)oxy)cyclohexyl)spiro[cyclopropane-1,5′-pyrrolo[2,3-d]pyrimidin]-6′(7′H)-one (7b)

To a stirred mixture of 6a (400 mg, 0.738 mmol, 1 equiv) in ACN (20 mL) was added 4 M HCl in dioxane (4 mL) in portions at 0° C. The resulting mixture was stirred for 1 h at room temperature. The reaction was monitored by TLC. The resulting mixture was concentrated under reduced pressure. The crude product (6b & 7b) was used in the next step directly without further purification. 6b: LCMS (ESI, m/z): [M+H]⁺ 428.2, 7b: LCMS (ESI, m/z): [M+H]⁺ 441.2.

### Step C. 2′-((1-((3-(bromomethyl)phenyl)sulfonyl)piperidin-4-yl)amino)-7′-((1R,3R)-3-hydroxy cyclohexyl)spiro[cyclopropane-1,5′-pyrrolo[2,3-d]pyrimidin]-6′(7′H)-one (Int-6) & 2′-((1-((3-(bromomethyl)phenyl)sulfonyl)piperidin-4-yl)amino)-7′-((1R,3R)-3-((tetrahydro-2H-pyran-2-yl)oxy) cyclohexyl)spiro[cyclopropane-1,5′-pyrrolo[2,3-d]pyrimidin]-6′(7′H)-one (Int-7)

To a stirred mixture of 6b and 7b (500 mg, 1.399 mmol, 1 equiv) and 3-(bromomethyl)benzenesulfonyl chloride (377.02 mg, 1.399 mmol, 1 equiv) in CH₂Cl₂ (5 mL) was added DIEA (903.93 mg, 6.995 mmol, 5 equiv) 0° C. The resulting mixture was stirred for 1 h at room temperature. The reaction was monitored by LCMS. The residue was purified by silica gel column chromatography, eluted with petroleum ether/EtOAc (1:1) to afford Int-6 (250 mg, 30.27%) and Int-7 (200 mg, 21.19%) as a white solid. Int-6: LCMS (ESI, m/z): [M+H]⁺ 589.1, Int-7: LCMS (ESI, m/z): [M+H]⁺ 673.2.

### 2′-((1-((3-(bromomethyl)phenyl) sulfonyl)piperidin-4-yl)amino)-7′-((1R,3R)-3-((tetrahydro-2H-pyran-2-yl)oxy)cyclohexyl)spiro [cyclopropane-1,5′-pyrrolo[2,3-d]pyrimidin]-6′(7′H)-one (Int-7)

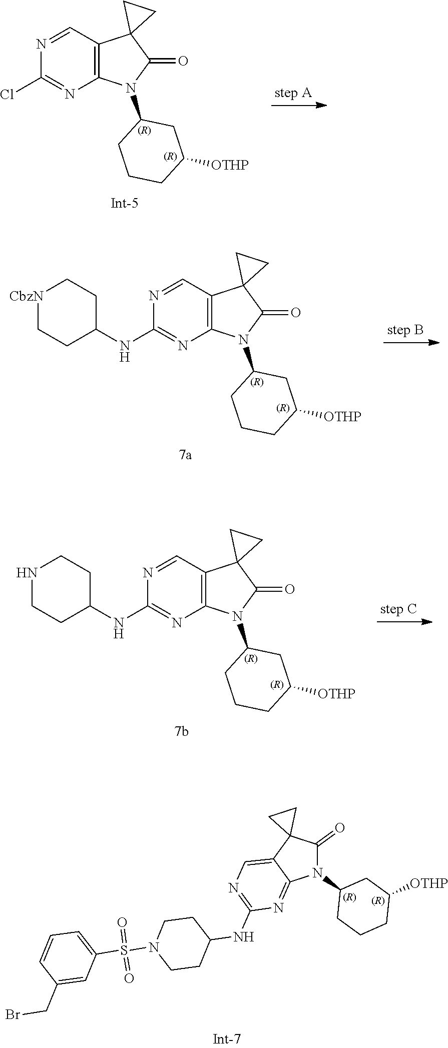

### Step A. Benzyl 4-{7′-[(1R,3R)-3-(oxan-2-yloxy)cyclohexyl]-6′-oxospiro[cyclopropane-1,5′-pyrrolo[2,3-d]pyrimidin]-2′-ylamino}piperidine-1-carboxylate (7a)

To a mixture of Int-5 (2 g, 5.293 mmol, 1 equiv) and benzyl 4-aminopiperidine-1-carboxylate (1.49 g, 6.352 mmol, 1.2 equiv) in dioxane (20 mL) was added Pd₂(dba)₃ (0.48 g, 0.529 mmol, 0.1 equiv) and RuPhos (0.49 g, 1.059 mmol, 0.2 equiv) and Cs₂CO₃ (5.17 g, 15.879 mmol, 3 equiv). The resulting mixture was stirred for 2 h at 100° C. The resulting mixture was concentrated under reduced pressure. The residue was purified by silica gel column chromatography, eluted with EA/PE (1:1) to afford 7a (1.5 g, 49.23%) as a brown solid. LCMS (ESI, m/z): [M+H]⁺=576.

### Step B. 7′-[(1R,3R)-3-(Oxan-2-yloxy)cyclohexyl]-2′-(piperidin-4-ylamino)spiro[cyclopropane-1,5′-pyrrolo[2,3-d]pyrimidin]-6′-one (7b)

To a solution of 7a (6 g, 10.422 mmol, 1 equiv) in CF₃CH₂OH (60 mL) was added Pd(OH)₂/C (3 g) under nitrogen atmosphere. The mixture was hydrogenated at room temperature for 2 h under hydrogen atmosphere using a hydrogen balloon. The resulting mixture was filtered, the filter cake was washed with ACN (3×50 ml). The filtrate was concentrated under reduced pressure. The crude product was used in the next step directly without further purification. LCMS (ESI, m/z): [M+H]⁺=442.

### Step C. 2′-((1-((3-(Bromomethyl)phenyl)sulfonyl)piperidin-4-yl)amino)-7′-((1R,3 R)-3-((tetrahydro-2H-pyran-2-yl)oxy) cyclohexyl)spiro[cyclopropane-1,5′-pyrrolo[2,3-d]pyrimidin]-6′(7′H)-one (Int-7)

To a solution of 7b (1.64 g, 3.710 mmol, 1 equiv) and DIEA (1.44 g, 11.130 mmol, 3 equiv) in DCM (20 mL) was added 3-(bromomethyl)benzenesulfonyl chloride (1 g, 3.710 mmol, 1.00 equiv) at 0° C. under nitrogen atmosphere. The resulting mixture was stirred for 2 h at 0° C. under nitrogen atmosphere. The reaction was quenched by the addition of water at 0° C. The aqueous layer was extracted with CH₂Cl₂ (2×60 mL). The combined organic layers were washed with brine, dried over anhydrous Na₂SO₄. The crude product was used in the next step directly without further purification. This resulted in Int-7 (3g, crude) as a brown solid. LCMS (ESI, m/z): [M+H]⁺=674.

### 4-{7′-[(1R,3R)-3-(Oxan-2-yloxy)cyclohexyl]-6′-oxospiro[cyclopropane-1,5′-pyrrolo[2,3-d]pyrimidin]-2′-ylamino}benzenesulfonyl chloride (Int-8)

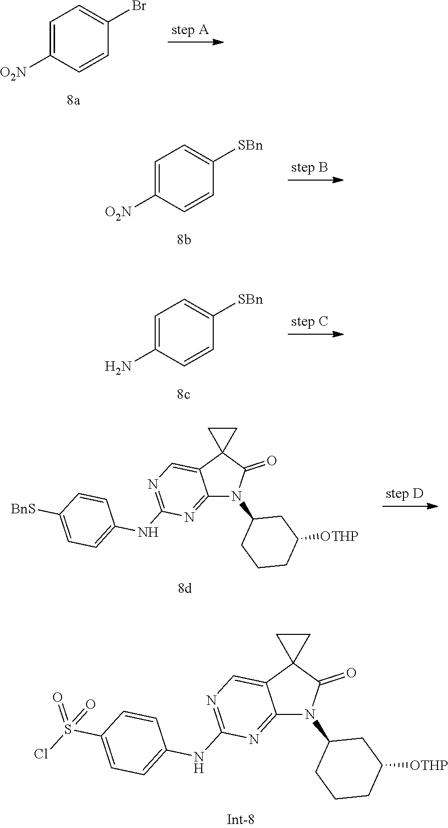

### Step A. 1-(Benzylsulfanyl)-4-nitrobenzene (8b)

A solution of 8a (2 g, 9.901 mmol, 1 equiv) and KOH (1.11 g, 19.802 mmol, 2 equiv) in H₂O (20 mL) was treated with TBAB (3.19 g, 9.901 mmol, 1 equiv), L-proline (227.98 mg, 1.980 mmol, 0.2 equiv) and FeCl₃·6H₂O (267.60 mg, 0.990 mmol, 0.1 equiv) at room temperature under nitrogen atmosphere followed by the addition of benzyl mercaptan (983.73 mg, 7.921 mmol, 0.8 equiv) dropwise at room temperature. The resulting mixture was stirred for overnight at 130° C. Desired product could be detected by LCMS. The resulting mixture was concentrated under reduced pressure. The residue was purified by silica gel column chromatography, eluted with petroleum ether/EtOAc (10:1) to afford 8b (860 mg, 34.95%) as a dark blue oil. LCMS (ESI, m/z): [M+H]⁺ 146.0

### Step B. 4-(Benzylsulfanyl)aniline (8c)

To a stirred solution of 8b (860 mg, 3.506 mmol, 1 equiv) in EtOH (15 mL) was added Fe (978.94 mg, 17.530 mmol, 5 equiv) and statured NH₄Cl aqueous (1.5 mL) at room temperature. The resulting mixture was stirred for 1 h at 80° C. Mainly product could be detected by LCMS. The resulting mixture was filtered, the filter cake was washed with EtOH (6×70 mL). The filtrate was concentrated under reduced pressure. The residue was purified by silica gel column chromatography, eluted with petroleum ether/EtOAc (10:1) to afford 8c (750 mg, 83.26%) as a yellow oil. LCMS (ESI, m/z): [M+H]⁺ 216.0.

### Step C. 2′-{[4-(Benzylsulfanyl)phenyl]amino}-7′-[(1R,3R)-3-(oxan-2-yloxy)cyclohexyl]spiro [cyclopropane-1,5′-pyrrolo[2,3-d]pyrimidin]-6′-one (8d)

To a solution of Int-5 (510 mg, 1.350 mmol, 1 equiv) and 8c (435.90 mg, 2.025 mmol, 1.5 equiv) in dioxane (2 mL) were added BINAP (168.08 mg, 0.270 mmol, 0.2 equiv), Cs₂CO₃ (1319.24 mg, 4.050 mmol, 3 equiv) and RuPhos Palladacycle Gen.3 (169.33 mg, 0.203 mmol, 0.15 equiv). After stirring for 2 h at 90° C. under a nitrogen atmosphere, the resulting mixture was concentrated under reduced pressure. The residue was purified by silica gel column chromatography, eluted with petroleum ether/EtOAc (1:1) to afford 8d (630 mg, 83.84%) as a yellow solid. LCMS (ESI, m/z): [M+H]⁺ 557.2.

### Step D. 4-{7′-[(1R,3R)-3-(Oxan-2-yloxy)cyclohexyl]-6′-oxospiro[cyclopropane-1,5′-pyrrolo[2,3-d]pyrimidin]-2′-ylamino}benzenesulfonyl chloride (Int-8)

To a stirred solution of 8d (200 mg, 0.359 mmol, 1 equiv) and HOAc (86.29 mg, 1.437 mmol, 4.00 equiv) in CH₂Cl₂ (8 mL) and H₂O (26.53 mg, 1.472 mmol, 4.1 equiv) was added SO₂Cl₂ (198.78 mg, 1.472 mmol, 4.1 equiv) dropwise at −80° C. The resulting mixture was stirred for 30 min at −80° C. Desired product could be detected by LCMS. The mixture was basified to pH 7 with saturated NaHCO₃ (aq.). The combined organic layers were dried over anhydrous sodium sulfate. After filtration, the filtrate was concentrated under reduced pressure. The crude product Int-8 was used in the next step directly without further purification. LCMS (ESI, m/z): [M+H]⁺ 533.1.

### 1-Bromo-3-((4-nitrophenyl)sulfonyl)benzene (Int-9)

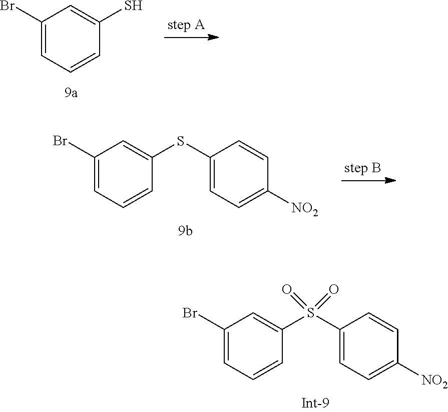 [SMILES: Sc1cccc(Br)c1 ; O=[N+]([O-])c1ccc(Sc2cccc(Br)c2)cc1 ; O=[N+]([O-])c1ccc(S(=O)(=O)c2cccc(Br)c2)cc1]

### Step A. 1-Bromo-3-[(4-nitrophenyl) sulfanyl]benzene (9b)

To a stirred solution of 9a (10 g, 52.890 mmol, 1 equiv) and benzene, 1-iodo-4-nitro-(13.17 g, 52.890 mmol, 1 equiv) in DMSO (100 mL) was added K₂CO₃ (10.96 g, 79.335 mmol, 1.5 equiv) at room temperature. The resulting mixture was stirred for overnight at 90° C. The mixture was allowed to cool down to room temperature. Desired product could be detected by LCMS. The resulting mixture was diluted with water (100 mL) and extracted with EtOAc (3×100 mL). The combined organic layers were washed with brine (100 mL), dried over anhydrous Na₂SO₄. After filtration, the filtrate was concentrated under reduced pressure. The residue was purified by silica gel column chromatography, eluted with petroleum ether/EtOAc (1:1) to afford 9b (16 g, 97.53%) as a yellow solid. LCMS (ESI, m/z): [M+H]⁺ 310.3.

### Step B. 1-Bromo-3-((4-nitrophenyl)sulfonyl)benzene (Int-9)

A solution of 9b (16 g, 51.585 mmol, 1 equiv) and m-CPBA (26.70 g, 154.755 mmol, 3 equiv) in CH₂Cl₂ (160 mL) was stirred for overnight at room temperature. Desired product could be detected by TLC (petroleum ether/EtOAc=5:1, R_{f}=0.5). The reaction was quenched by the addition of sat. sodium hyposulfite (aq.) (100 mL) at room temperature. The resulting mixture was diluted with water (200 mL) and extracted with CH₂Cl₂ (3×200 mL). The combined organic layers were washed with brine (200 mL), dried over anhydrous Na₂SO₄. After filtration, the filtrate was concentrated under reduced pressure. The residue was purified by silica gel column chromatography, eluted with petroleum ether/EtOAc (5:1) to afford Int-9 (16.9 g, 95.75%) as a yellow solid.

### 2′-({1-[3-(methylamino)azetidin-1-ylsulfonyl]piperidin-4-yl}amino)-7′-(2-methylcyclopentyl)spiro [cyclopropane-1,5′-pyrrolo[2,3-d]pyrimidin]-6′-one (Int-10)

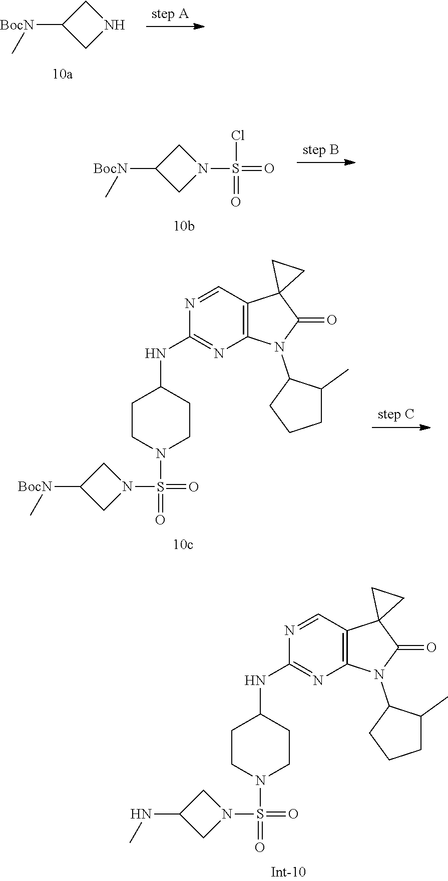

### Step A. Tert-butyl N-[1-(chlorosulfonyl)azetidin-3-yl]-N-methylcarbamate (10b)

To a stirred solution of SO₂Cl₂ (218.16 mg, 1.616 mmol, 1.2 equiv) in DCM (5 mL) was added the solution of 10a (300 mg, 1.347 mmol, 1 equiv) and pyridine (319.65 mg, 4.041 mmol, 3 equiv) in DCM (2 mL) dropwise at 0° C. The resulting mixture was stirred for overnight at room temperature. The mixture was acidified with 1N HCl. The resulting mixture was extracted with DCM. The combined organic layers were washed with brine, dried over anhydrous Na₂SO₄. After filtration, the filtrate was concentrated under reduced pressure to afford 10b (330 mg, 86.03%) as a yellow solid.

### Step B. Tert-butyl N-methyl-N-(1-{4-[7′-(2-methylcyclopentyl)-6′-oxospiro[cyclopropane-1,5′-pyrrolo[2,3-d]pyrimidin]-2′-ylamino]piperidin-1-ylsulfonyl}azetidin-3-yl)carbamate (10c)

To a stirred solution of Int-2 (500 mg, 1.464 mmol, 1 equiv) and Et₃N (4.45 g, 43.920 mmol, 30 equiv) in THF (10 mL) was added 10b (625.46 mg, 2.196 mmol, 1.5 equiv) at 0° C. The resulting mixture was stirred for 2 h at room temperature. Desired product could be detected by LCMS. The resulting mixture was diluted with water (10 mL). The resulting mixture was extracted with EtOAc (3×10 mL). The combined organic layers were dried over anhydrous Na₂SO₄. After filtration, the filtrate was concentrated under reduced pressure. The residue was purified by silica gel column chromatography, eluted with petroleum ether/EtOAc (1:3) to afford 10c (600 mg, 62.95%) as a white solid. LCMS (ESI, m/z): [M+H]⁺ 590.3.

### Step C. 2′-({1-[3-(methylamino)azetidin-1-ylsulfonyl]piperidin-4-yl}amino)-7′-(2-methylcyclopentyl) spiro [cyclopropane-1,5′-pyrrolo[2,3-d]pyrimidin]-6′-one (Int-10)

A solution of 10c (60 mg, 0.102 mmol, 1 equiv) in TFA (2 mL) was stirred for 2 h at room temperature under nitrogen atmosphere. Desired product could be detected by LCMS. The resulting mixture was concentrated under reduced pressure. The crude product was purified by Prep-HPLC with the following conditions (Column: YMC-Actus Triart C18 EXRS 30*150 mm, 5 μm; Mobile Phase A: water (10 mmol/L NH₄HCO₃), Mobile Phase B: ACN; Flow rate: 60 mL/min mL/min; Gradient: 21% B to 37% B in 8 min; Wave Length: 254 nm/220 nm nm; RT1 (min): 11.97) to afford Int-10 (18 mg, 35.23%) as a white solid. LCMS (ESI, m/z): [M+H]⁺ 490.2.

### 2′-[(1-{hexahydro-1H-pyrrolo[3,4-c]pyrrole-2-sulfonyl}piperidin-4-yl)amino]-7′-(2-methylcyclopentyl) spiro[cyclopropane-1,5′-pyrrolo[2,3-d]pyrimidin]-6′-one (Int-11)

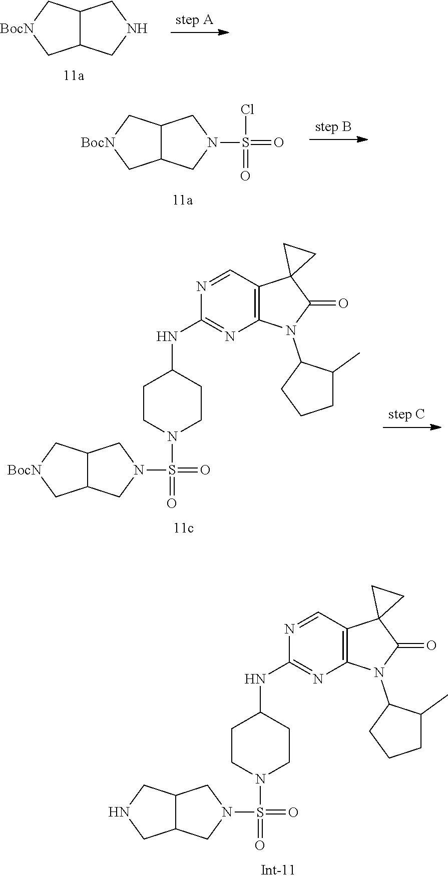 [SMILES: CC(C)(C)OC(=O)N1CC2CNCC2C1 ; CC(C)(C)OC(=O)N1CC2CN(S(=O)(=O)Cl)CC2C1 ; CC1CCCC1N1C(=O)C2(CC2)c2cnc(NC3CCN(S(=O)(=O)N4CC5CN(C(=O)OC(C)(C)C)CC5C4)CC3)nc21 ; CC1CCCC1N1C(=O)C2(CC2)c2cnc(NC3CCN(S(=O)(=O)N4CC5CNCC5C4)CC3)nc21]

### Step A. Tert-butyl 6-(chlorosulfonyl)-2,6-diazaspiro[3.3]heptane-2-carboxylate (11b)

To a stirred solution of 11a (100 mg, 0.504 mmol, 1 equiv) and pyridine (59.84 mg, 0.756 mmol, 1.5 equiv) in DCM (2 mL) was added sulfonyl chloride (81.68 mg, 0.605 mmol, 1.2 equiv) dropwise at 0° C. The resulting mixture was stirred for 1 h at room temperature under nitrogen atmosphere. The mixture was acidified with 1N HCl. The resulting mixture was extracted with DCM. The combined organic layers were washed with brine, dried over anhydrous Na₂SO₄. After filtration, the filtrate was concentrated under reduced pressure to afford 11b. The crude product was used in the next step directly without further purification.

### Step B. Tert-butyl 5-{4-[7′-(2-methylcyclopentyl)-6′-oxospiro[cyclopropane-1,5′-pyrrolo[2,3-d]pyrimidin]-2′-ylamino]piperidin-1-ylsulfonyl}-hexahydropyrrolo[3,4-c]pyrrole-2-carboxylate (11c)

To a stirred solution of Int-2 (95 mg, 0.278 mmol, 1.00 equiv) and DIEA (359.59 mg, 2.780 mmol, 10 equiv) in THF (2 mL) was added 11b (103.76 mg, 0.334 mmol, 1.2 equiv) at 0° C. The resulting mixture was stirred for 1 h at room temperature under nitrogen atmosphere. Desired product could be detected by LCMS. The resulting mixture was diluted with water (10 mL). The resulting mixture was extracted with EtOAc (3×10 mL). The combined organic layers were dried over anhydrous Na₂SO₄. After filtration, the filtrate was concentrated under reduced pressure. The residue was purified by Prep-TLC (CH₂Cl₂/MeOH 20:1) to afford 11c (60 mg, 32.39%) as a white solid. LCMS (ESI, m/z): [M+H]⁺ 616.3.

### Step C. 2′-[(1-{hexahydro-1H-pyrrolo[3,4-c]pyrrole-2-sulfonyl}piperidin-4-yl)amino]-7′-(2-methylcyclopentyl)spiro[cyclopropane-1,5′-pyrrolo[2,3-d]pyrimidin]-6′-one (Int-11)

To a stirred solution of 11c (60 mg, 0.097 mmol, 1 equiv) in MeCN (1.5 mL) was added 4M HCl (gas) in 1,4-dioxane (0.3 mL, 1.200 mmol, 12.32 equiv) dropwise at 0° C. The resulting mixture was stirred for 1 h at 0° C. Desired product could be detected by LCMS. The resulting mixture was concentrated under reduced pressure. The resulting mixture was diluted with DCM. The mixture was neutralized to pH 7 with Et₃N. The residue was purified by reversed-phase flash chromatography to afford Int-11 (10.5 mg, 20.71%) as a white solid. LCMS (ESI, m/z): [M+H]⁺ 516.3.

### 2′-[(1-{1,7-diazaspiro[4.4]nonane-1-sulfonyl}piperidin-4-yl)amino]-7′-(2-methylcyclopentyl)spiro [cyclopropane-1,5′-pyrrolo[2,3-d]pyrimidin]-6′-one (Int-12)

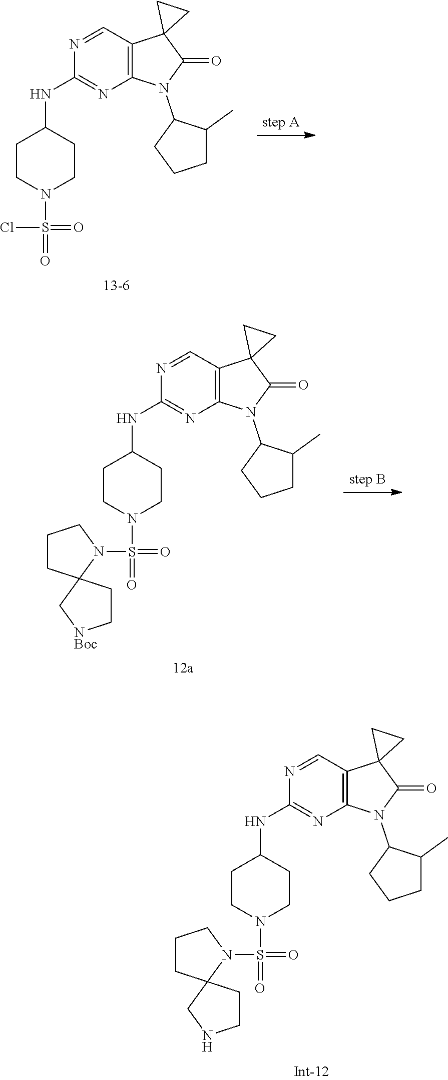 [SMILES: CC1CCCC1N1C(=O)C2(CC2)c2cnc(NC3CCN(S(=O)(=O)Cl)CC3)nc21 ; CC1CCCC1N1C(=O)C2(CC2)c2cnc(NC3CCN(S(=O)(=O)N4CCCC45CCN(C(=O)OC(C)(C)C)C5)CC3)nc21 ; CC1CCCC1N1C(=O)C2(CC2)c2cnc(NC3CCN(S(=O)(=O)N4CCCC45CCNC5)CC3)nc21]

### Step A. Tert-butyl 1-{4-[7′-(2-methylcyclopentyl)-6′-oxospiro[cyclopropane-1,5′-pyrrolo[2,3-d]pyrimidin]-2′-ylamino]piperidin-1-ylsulfonyl}-1,7-diazaspiro[4.4]nonane-7-carboxylate (12a)

To a mixture of 13-6 (550 mg, 1.250 mmol, 1 equiv) and tert-butyl 1,7-diazaspiro[4.4]nonane-7-carboxylate (565.85 mg, 2.500 mmol, 2 equiv) in MeCN (1 mL) was added DIEA (646.29 mg, 5.000 mmol, 4 equiv) at room temperature. The resulting mixture was stirred for 2 h at 90° C. under nitrogen atmosphere. Desired product could be detected by LCMS. The resulting mixture was concentrated under reduced pressure. The residue was purified by silica gel column chromatography, eluted with petroleum ether/EtOAc (1:1) to afford 12a (520 mg, 59.44%) as a light yellow solid. LCMS (ESI, m/z): [M+H]⁺ 630.3.

### Step B. 2′-[(1-{1,7-diazaspiro[4.4]nonane-1-sulfonyl}piperidin-4-yl)amino]-7′-(2-methylcyclopentyl) spiro[cyclopropane-1,5′-pyrrolo[2,3-d]pyrimidin]-6′-one (Int-12)

To a stirred mixture of 12a (40 mg, 0.064 mmol, 1 equiv) in MeCN (1 mL) was added 4M HCl (gas) in 1,4-dioxane (0.2 mL) dropwise at 0° C. The resulting mixture was stirred for 1 h at 0° C. Desired product could be detected by LCMS. The resulting mixture was concentrated under reduced pressure. The resulting mixture was diluted with DCM. The mixture was neutralized to pH 7 with Et₃N. The crude product was purified by Prep-HPLC to afford Int-12 (18 mg, 52.76%) as a white solid. LCMS (ESI, m/z): [M+H]⁺ 530.2.

### 2′-[(1-{2,5-Diazaspiro[3.5]nonane-2-sulfonyl}piperidin-4-yl)amino]-7′-(2-methylcyclopentyl)spiro [cyclopropane-1,5′-pyrrolo[2,3-d]pyrimidin]-6′-one (Int-13)

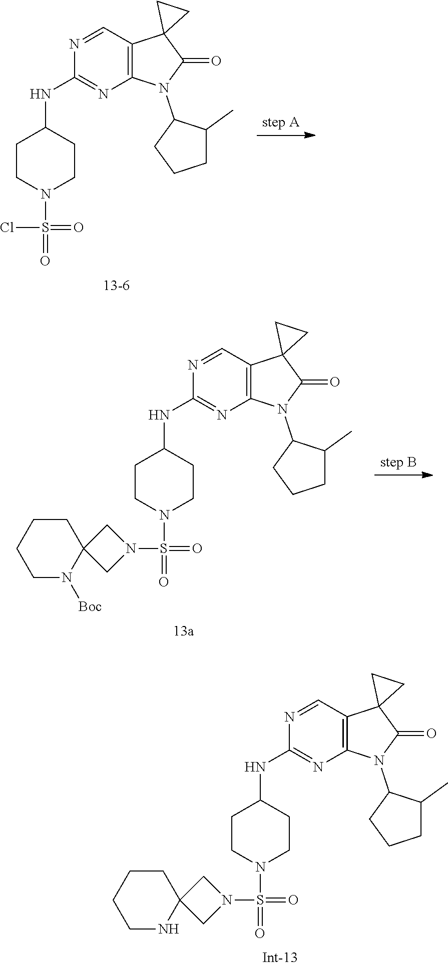 [SMILES: CC1CCCC1N1C(=O)C2(CC2)c2cnc(NC3CCN(S(=O)(=O)Cl)CC3)nc21 ; CC1CCCC1N1C(=O)C2(CC2)c2cnc(NC3CCN(S(=O)(=O)N4CC5(CCCCN5C(=O)OC(C)(C)C)C4)CC3)nc21 ; CC1CCCC1N1C(=O)C2(CC2)c2cnc(NC3CCN(S(=O)(=O)N4CC5(CCCCN5)C4)CC3)nc21]

### Step A. Tert-butyl 2-{4-[7′-(2-methylcyclopentyl)-6′-oxospiro[cyclopropane-1,5′-pyrrolo[2,3-d]pyrimidin]-2′-ylamino]piperidin-1-ylsulfonyl}-2,5-diazaspiro[3.5]nonane-5-carboxylate (13a)

To a mixture of tert-butyl 2,5-diazaspiro[3.5]nonane-5-carboxylate (257.21 mg, 1.136 mmol, 1 equiv) and TEA (1150.03 mg, 11.360 mmol, 10 equiv) in DCM (5 mL) were added 13-6 (500 mg, 1.136 mmol, 1 equiv) at 0° C. The resulting mixture was stirred for 2 h at room temperature under nitrogen atmosphere. Desired product could be detected by LCMS. The resulting mixture was diluted with water (10 mL). The resulting mixture was extracted with CH₂Cl₂ (3×10 mL). The combined organic layers were dried over anhydrous Na₂SO₄. After filtration, the filtrate was concentrated under reduced pressure. The residue was purified by silica gel column chromatography, eluted with petroleum ether/EtOAc (1:1) to afford 13a (610 mg, 85.22%) as a brown solid. LCMS (ESI, m/z): [M+H]⁺ 630.3.

### Step B. 2′-[(1-{2,5-Diazaspiro[3.5]nonane-2-sulfonyl}piperidin-4-yl)amino]-7′-(2-methylcyclopentyl) spiro[cyclopropane-1,5′-pyrrolo[2,3-d]pyrimidin]-6′-one (Int-13)

To a stirred solution of 13a (40 mg, 0.064 mmol, 1 equiv) and Et₃N (64.27 mg, 0.640 mmol, 10 equiv) in anhydrous DCM (2 mL) was added TMSOTf (70.58 mg, 0.320 mmol, 5 equiv) at 0° C. and stirred for 1 h. Desired product could be detected by LCMS. The reaction was quenched with water. The aqueous layer was extracted with DCM (2×15 ml). The resulting mixture was concentrated under reduced pressure. The residue was purified by reversed-phase flash chromatography with the following conditions: Column: Xbridge Phenyl OBD Column, 19*150 mm, 5 m; Mobile Phase A: Water (10 mmol/L NH₄HCO₃, Mobile Phase B: ACN; Flow rate: 60 mL/min; Gradient: 33% B to 48% B in 10 min; Wave Length: 254 nm/220 nm. This resulted in Int-13 (11.2 mg, 33.16%) as a white solid. LCMS (ESI, m/z): [M+H]⁺ 530.2

### 7′-(2-Methylcyclopentyl)-2′-{[1-(piperazine-1-sulfonyl)piperidin-4-yl]amino}spiro[cyclopropane-1,5′-pyrrolo[2,3-d]pyrimidin]-6′-one (Int-14)

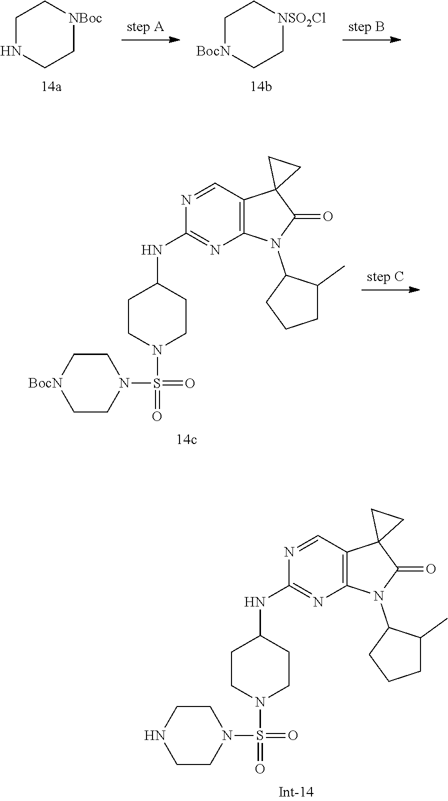

### Step A. tert-butyl 3-(acetylsulfanyl) piperidine-1-carboxylate (14b)

To a stirred solution of sulfonyl chloride (0.87 g, 6.443 mmol, 1.2 equiv) in DCM (10 mL) was added the solution of 14a (1 g, 5.369 mmol, 1 equiv) and pyridine (0.64 g, 8.053 mmol, 1.5 equiv) in DCM (5 mL) dropwise at 0° C. The resulting mixture was stirred for overnight at room temperature. The mixture was acidified with 1N HCl. The resulting mixture was extracted with DCM. The combined organic layers were washed with brine, dried over anhydrous Na₂SO₄. After filtration, the filtrate was concentrated under reduced pressure. The crude product 14b (1.2 g) was used in the next step directly without further purification.

### Step B. Tert-butyl 4-{4-[7′-(2-methylcyclopentyl)-6′-oxospiro[cyclopropane-1,5′-pyrrolo[2,3-d]pyrimidin]-2′-ylamino]piperidin-1-ylsulfonyl}piperazine-1-carboxylate (14c)

To a stirred solution of Int-2 (100 mg, 0.293 mmol, 1 equiv) and Et₃N (889.06 mg, 8.790 mmol, 30 equiv) in THF (2 mL) was added 14b (166.79 mg, 0.586 mmol, 2 equiv) at 0° C. The resulting mixture was stirred for 2 h at room temperature. Desired product could be detected by LCMS. The resulting mixture was diluted with water (2 mL). The resulting mixture was extracted with EtOAc (3×10 mL). The combined organic layers were dried over anhydrous Na₂SO₄. After filtration, the filtrate was concentrated under reduced pressure. The residue was purified by Prep-TLC (CH₂Cl₂/MeOH 20:1) to afford 14c (150 mg, 78.60%) as a white solid. LCMS (ESI, m/z): [M+H]⁺ 590.3.

### Step C. 7′-(2-Methylcyclopentyl)-2′-{[1-(piperazine-1-sulfonyl)piperidin-4-yl]amino}spiro [cyclopropane-1,5′-pyrrolo[2,3-d]pyrimidin]-6′-one (Int-14)

Into a 8 mL vial were added 14c (100 mg, 0.170 mmol, 1 equiv), MeCN (2.5 mL) and 4 M HCl (gas) in 1,4-dioxane (0.5 mL) at 0° C. The resulting mixture was stirred for 30 min at 0° C. Desired product could be detected by LCMS. The mixture was basified to pH 8 with Et₃N. The resulting mixture was concentrated under reduced pressure. The residue was purified by reversed-phase flash chromatography with the following conditions: column, C18 silica gel; mobile phase, MeCN in water (10 mmol/L NH₄HCO₃), 40% to 60% gradient in 20 min; detector, UV 254 nm. This resulted in Int-14 (15.1 mg, 17.91%) as a white solid. LCMS (ESI, m/z): [M+H]⁺ 490.3.

### 2′-[(1-{2,6-Diazaspiro[3.5]nonane-2-sulfonyl}piperidin-4-yl)amino]-7′-(2-methylcyclopentyl)spiro [cyclopropane-1,5′-pyrrolo[2,3-d]pyrimidin]-6′-one (Int-15)

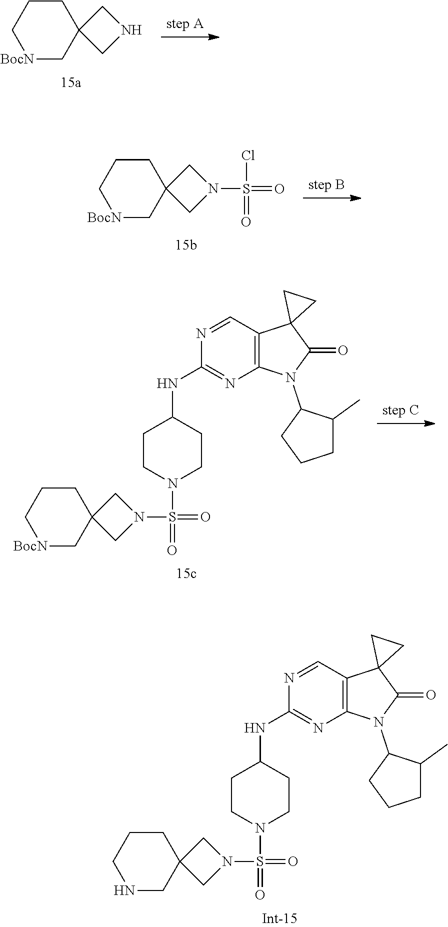 [SMILES: CC(C)(C)OC(=O)N1CCCC2(CNC2)C1 ; CC(C)(C)OC(=O)N1CCCC2(C1)CN(S(=O)(=O)Cl)C2 ; CC1CCCC1N1C(=O)C2(CC2)c2cnc(NC3CCN(S(=O)(=O)N4CC5(CCCN(C(=O)OC(C)(C)C)C5)C4)CC3)nc21 ; CC1CCCC1N1C(=O)C2(CC2)c2cnc(NC3CCN(S(=O)(=O)N4CC5(CCCNC5)C4)CC3)nc21]

### Step A. Tert-butyl 2-(chlorosulfonyl)-2,6-diazaspiro[3.5]nonane-6-carboxylate (17b)

To a stirred solution of SO₂Cl₂ (178.90 mg, 1.326 mmol, 1.5 equiv) in DCM (5 mL) was added the solution of 15a (200 mg, 0.884 mmol, 1 equiv) and pyridine (139.80 mg, 1.768 mmol, 2 equiv) in DCM (2 mL) dropwise at 0° C. The resulting mixture was stirred for overnight at room temperature. The mixture was acidified with 1N HCl. The resulting mixture was extracted with DCM. The combined organic layers were washed with brine, dried over anhydrous Na₂SO₄. After filtration, the filtrate was concentrated under reduced pressure to afford 15b (240 mg, 83.61%) as a yellow solid.

### Step B. Tert-butyl 2-{4-[7′-(2-methylcyclopentyl)-6′-oxospiro[cyclopropane-1,5′-pyrrolo[2,3-d]pyrimidin]-2′-ylamino]piperidin-1-ylsulfonyl}-2,6-diazaspiro[3.5]nonane-6-carboxylate (15c)

To a stirred solution of 15b (500 mg, 1.539 mmol, 1 equiv) and Et₃N (4.67 g, 46.170 mmol, 30 equiv) in THF (10 mL) was added Int-2 (1.05 g, 3.078 mmol, 2 equiv) at room temperature. The resulting mixture was stirred for 2 h at room temperature. Desired product could be detected by LCMS. The resulting mixture was concentrated under reduced pressure. The residue was purified by silica gel column chromatography, eluted with CH₂Cl₂/MeOH (30:1) to afford 15c (300 mg, 27.32%) as a yellow solid. LCMS (ESI, m/z): [M+H]⁺ 630.3

### Step C. 2′-[(1-{2,6-Diazaspiro[3.5]nonane-2-sulfonyl}piperidin-4-yl)amino]-7′-(2-methylcyclopentyl) spiro [cyclopropane-1,5′-pyrrolo[2,3-d]pyrimidin]-6′-one (Int-15)

To a stirred solution of 15c (50 mg, 0.079 mmol, 1 equiv) in CH₃CN (2.5 mL) was added 4 M HCl (gas) in 1,4-dioxane (0.5 mL) dropwise at 0° C. The resulting mixture was stirred for 30 min at 0° C. Desired product could be detected by LCMS. The resulting mixture was concentrated under reduced pressure. The mixture was basified to pH 7 with Et₃N. The resulting mixture was concentrated under reduced pressure. The crude product was purified by Prep-HPLC with the following conditions (Column: XBridge Shield RP18 OBD Column, 30*150 mm, 5 μm; Mobile Phase A: water (10 mmol/L NH₄HCO₃+0.05% NH₃·H₂O), Mobile Phase B: ACN; Flow rate: 60 mL/min mL/min; Gradient: 32% B to 45% B in 10 min; Wave Length: 254 nm/220 nm nm; RT1 (min): 9.2) to afford Int-15 (7.5 mg, 17.01%) as a white solid. LCMS (ESI, m/z): [M+H]⁺ 530.3

### 2′-[(1-{2,6-Diazaspiro[3.3]heptane-2-sulfonyl}piperidin-4-yl)amino]-7′-(2-methylcyclopentyl)spiro [cyclopropane-1,5′-pyrrolo[2,3-d]pyrimidin]-6′-one (Int-16)

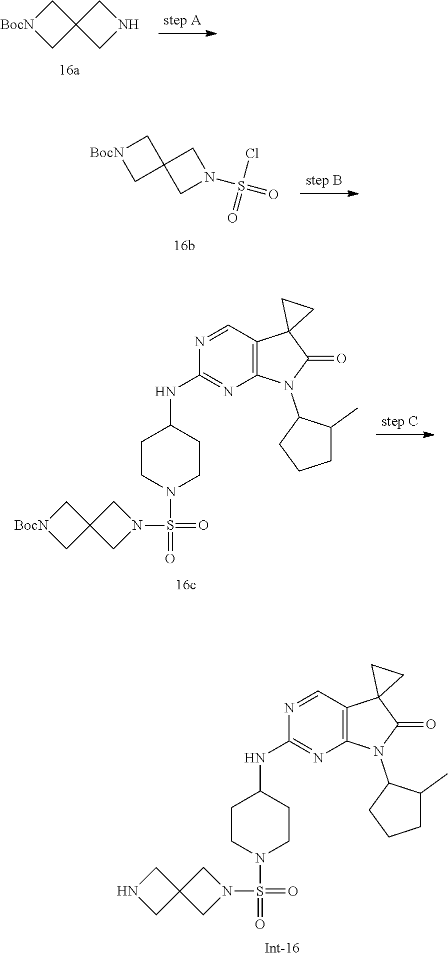 [SMILES: CC(C)(C)OC(=O)N1CC2(CNC2)C1 ; CC(C)(C)OC(=O)N1CC2(C1)CN(S(=O)(=O)Cl)C2 ; CC1CCCC1N1C(=O)C2(CC2)c2cnc(NC3CCN(S(=O)(=O)N4CC5(CN(C(=O)OC(C)(C)C)C5)C4)CC3)nc21 ; CC1CCCC1N1C(=O)C2(CC2)c2cnc(NC3CCN(S(=O)(=O)N4CC5(CNC5)C4)CC3)nc21]

### Step A. Tert-butyl 6-(chlorosulfonyl)-2,6-diazaspiro[3.3]heptane-2-carboxylate (16b)

To a stirred mixture of 16a (100 mg, 0.504 mmol, 1 equiv) in DCM (1 mL) were added SO₂Cl₂ (102.11 mg, 0.756 mmol, 1.5 equiv) and pyridine (79.79 mg, 1.008 mmol, 2 equiv) in portions at 0° C. The resulting mixture was stirred for overnight at room temperature. The mixture was acidified with 1N HCl. The resulting mixture was extracted with DCM. The combined organic layers were washed with brine, dried over anhydrous Na₂SO₄. After filtration, the filtrate was concentrated under reduced pressure. The crude product 16b (70 mg) was used in the next step directly without further purification.

### Step B. Tert-butyl 6-{4-[7′-(2-methylcyclopentyl)-6′-oxospiro[cyclopropane-1,5′-pyrrolo[2,3-d]pyrimidin]-2′-ylamino]piperidin-1-ylsulfonyl}-2,6-diazaspiro[3.3]heptane-2-carboxylate (16c)

To a mixture of 16b (70 mg, 0.236 mmol, 1 equiv) and Int-2 (96.65 mg, 0.283 mmol, 1.2 equiv) in THF (1 mL) were added Et₃N (71.61 mg, 0.708 mmol, 3 equiv) at room temperature. The resulting mixture was stirred for 2 h at room temperature. The reaction was monitored by LCMS. The residue was purified by silica gel column chromatography, eluted with petroleum ether/EtOAc (1/1) to afford 16c (70 mg, 49.32%) as a white solid. LCMS (ESI, m/z): [M+H]⁺ 602.3

### Step C. 2′-[(1-{2,6-Diazaspiro[3.3]heptane-2-sulfonyl}piperidin-4-yl)amino]-7′-(2-methylcyclopentyl) spiro[cyclopropane-1,5′-pyrrolo[2,3-d]pyrimidin]-6′-one (Int-16)

To a stirred solution of 16c (77 mg, 0.128 mmol, 1 equiv) in DCM (4 mL) was added TFA (0.8 mL) dropwise at 0° C. The resulting mixture was stirred for 30 min at room temperature. The reaction was monitored by LCMS. The resulting mixture was concentrated under reduced pressure. The mixture was basified to pH 8 with Et₃N. The crude product was purified by reverse phase flash with the following conditions (Column: XBridge Prep OBD C18 Column, 30*150 mm, 10 μm; Mobile Phase A: Water (10 mmol/L NH₄HCO₃+0.05% NH3H2O), Mobile Phase B: MEOH; Flow rate: 60 mL/min mL/min; Gradient: 53% B to 68% B in 8 min; Wave Length: 254 nm/220 nm nm; RT1 (min): 8.05) to Int-16 (17.7 mg, 27.57%) as a white solid. LCMS (ESI, m/z): [M+H]⁺ 502.2

### 2′-[(1-{2,7-Diazaspiro[3.5]nonane-2-sulfonyl}piperidin-4-yl)amino]-7′-(2-methylcyclopentyl)spiro [cyclopropane-1,5′-pyrrolo[2,3-d]pyrimidin]-6′-one (Int-17)

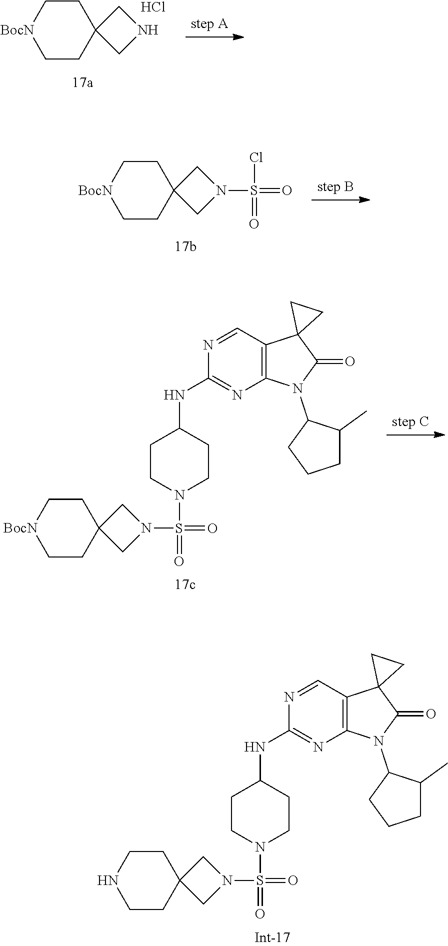 [SMILES: CC(C)(C)OC(=O)N1CCC2(CC1)CNC2 ; Cl ; CC(C)(C)OC(=O)N1CCC2(CC1)CN(S(=O)(=O)Cl)C2 ; CC1CCCC1N1C(=O)C2(CC2)c2cnc(NC3CCN(S(=O)(=O)N4CC5(CCN(C(=O)OC(C)(C)C)CC5)C4)CC3)nc21 ; CC1CCCC1N1C(=O)C2(CC2)c2cnc(NC3CCN(S(=O)(=O)N4CC5(CCNCC5)C4)CC3)nc21]

### Step A. Tert-butyl 2-(chlorosulfonyl)-2,7-diazaspiro[3.5]nonane-7-carboxylate (17b)

To a stirred solution of SO₂Cl₂ (184.89 mg, 1.370 mmol, 1.2 equiv) in DCM (5 mL) was added the solution of 17a (300 mg, 1.142 mmol, 1 equiv) and pyridine (270.91 mg, 3.426 mmol, 3 equiv) in DCM (2 mL) dropwise at 0° C. The resulting mixture was stirred for overnight at room temperature. The mixture was acidified with 1N HCl. The resulting mixture was extracted with DCM. The combined organic layers were washed with brine, dried over anhydrous Na₂SO₄. After filtration, the filtrate was concentrated under reduced pressure to afford 17b (350 mg, 94.38%) as an off-white solid.

### Step B. Tert-butyl 2-{4-[7′-(2-methylcyclopentyl)-6′-oxospiro[cyclopropane-1,5′-pyrrolo[2,3-d]pyrimidin]-2′-ylamino]piperidin-1-ylsulfonyl}-2,7-diazaspiro[3.5]nonane-7-carboxylate (17c)

To a solution of Int-2 (500 mg, 1.464 mmol, 1 equiv) and Et₃N (4445.32 mg, 43.920 mmol, 30 equiv) in THF (5 mL) was added 17b (713.45 mg, 2.196 mmol, 1.5 equiv) at 0° C. The resulting mixture was stirred for 2 h at room temperature. Desired product could be detected by LCMS. The resulting mixture was concentrated under reduced pressure. The residue was purified by silica gel column chromatography, eluted with petroleum ether/EtOAc (1:1) to afford 17c (600 mg, 59.85%) as a white solid. LCMS (ESi, m/z): [M+H]⁺ 630.4.

### Step C. 2′-[(1-{2,7-Diazaspiro[3.5]nonane-2-sulfonyl}piperidin-4-yl)amino]-7′-(2-methylcyclopentyl) spiro [cyclopropane-1,5′-pyrrolo[2,3-d]pyrimidin]-6′-one (Int-17)

To a stirred solution of 17c (60 mg, 0.095 mmol, 1 equiv) in DCM (1 mL) was added CF₃COOH (0.2 mL, 2.693 mmol, 28.26 equiv) dropwise at 0° C. The resulting mixture was stirred for 1 h at 0° C. Desired product could be detected by LCMS. The resulting mixture was concentrated under reduced pressure. The mixture was neutralized to pH 7 with Et₃N. The residue was purified by reversed-phase flash chromatography to afford Int-17 (11.3 mg, 22.30%) as a white solid. LCMS (ESI, m/z): [M+H]⁺ 530.2

### 2′-[(1-{2,6-Diazabicyclo[3.2.0]heptane-6-sulfonyl}piperidin-4-yl)amino]-7′-(2-methylcyclopentyl)spiro [cyclopropane-1,5′-pyrrolo[2,3-d]pyrimidin]-6′-one (Int-18)

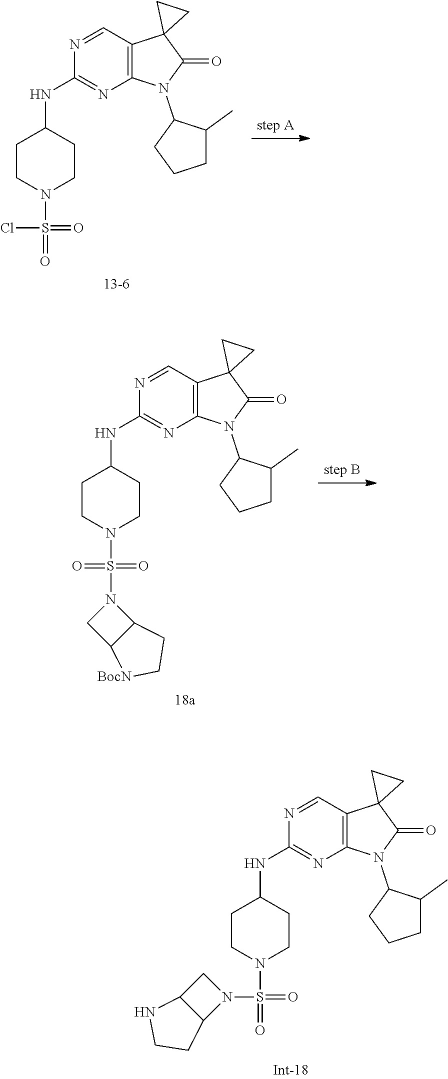 [SMILES: CC1CCCC1N1C(=O)C2(CC2)c2cnc(NC3CCN(S(=O)(=O)Cl)CC3)nc21 ; CC1CCCC1N1C(=O)C2(CC2)c2cnc(NC3CCN(S(=O)(=O)N4CC5C4CCN5C(=O)OC(C)(C)C)CC3)nc21 ; CC1CCCC1N1C(=O)C2(CC2)c2cnc(NC3CCN(S(=O)(=O)N4CC5NCCC54)CC3)nc21]

### Step A. Tert-butyl 6-{4-[7′-(2-methylcyclopentyl)-6′-oxospiro[cyclopropane-1,5′-pyrrolo[2,3-d]pyrimidin]-2′-ylamino]piperidin-1-ylsulfonyl}-2,6-diazabicyclo[3.2.0]heptane-2-carboxylate (18a)

To a mixture of tert-butyl 2,6-diazabicyclo[3.2.0]heptane-2-carboxylate (49.57 mg, 0.250 mmol, 1.1 equiv) and TEA (69.00 mg, 0.681 mmol, 3 equiv) in DCM (2 mL) were added 13-6 (100 mg, 0.227 mmol, 1 equiv) at room temperature. The resulting mixture was stirred for 2 h at room temperature. Desired product could be detected by LCMS. The resulting mixture was diluted with water (10 mL). The resulting mixture was extracted with CH2Cl2 (3×20 mL). The combined organic layers were dried over anhydrous Na₂SO₄. After filtration, the filtrate was concentrated under reduced pressure. The residue was purified by Prep-TLC (petroleum ether/EtOAc=1:1) to afford 18a (90 mg, 65.80%) as a brown solid. LCMS (ESI, m/z): [M+H]⁺ 602.3.

### Step B. 2′-[(1-{2,6-Diazabicyclo[3.2.0]heptane-6-sulfonyl}piperidin-4-yl)amino]-7′-(2-methylcyclopentyl)spiro [cyclopropane-1,5′-pyrrolo[2,3-d]pyrimidin]-6′-one (Int-18)

To a stirred solution of 18a (45 mg, 0.075 mmol, 1 equiv) in MeCN (3 mL) were added 4M HCl (gas) in 1,4-dioxane (0.6 mL) dropwise at 0° C. under air atmosphere. The resulting mixture was stirred for 1 h at 0° C. under air atmosphere. Desired product could be detected by LCMS. The resulting mixture was concentrated under reduced pressure. The residue was purified by reversed-phase flash chromatography with the following conditions: column, C18 silica gel; mobile phase, MeCN in Water (10 mmol/L NH₄HCO₃), 10% to 70% gradient in 10 min; detector, UV 254 nm. This resulted in Int-18 (20.3 mg, 52.33%) as an off-white solid. LCMS (ESI, m/z): [M+H]⁺ 502.3.

### 2′-[(1-{3,6-Diazabicyclo[3.2.0]heptane-6-sulfonyl}piperidin-4-yl)amino]-7′-(2-methylcyclopentyl)spiro [cyclopropane-1,5′-pyrrolo[2,3-d]pyrimidin]-6′-one (Int-19)

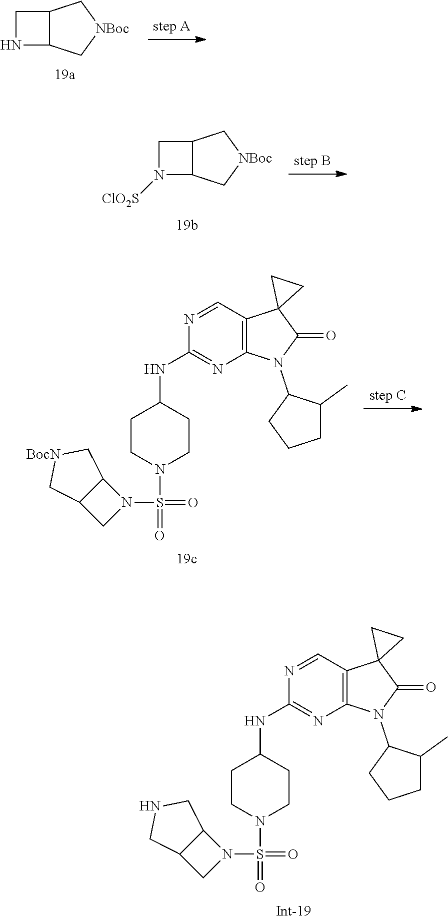 [SMILES: CC(C)(C)OC(=O)N1CC2CNC2C1 ; CC(C)(C)OC(=O)N1CC2CN(S(=O)(=O)Cl)C2C1 ; CC1CCCC1N1C(=O)C2(CC2)c2cnc(NC3CCN(S(=O)(=O)N4CC5CN(C(=O)OC(C)(C)C)CC54)CC3)nc21 ; CC1CCCC1N1C(=O)C2(CC2)c2cnc(NC3CCN(S(=O)(=O)N4CC5CNCC54)CC3)nc21]

### Step A. Tert-butyl 6-(chlorosulfonyl)-3,6-diazabicyclo[3.2.0]heptane-3-carboxylate (19b)

To a stirred mixture of 19a (500 mg, 2.522 mmol, 1 equiv) and pyridine (398.96 mg, 5.044 mmol, 2 equiv) in DCM (5 mL) was added SO₂Cl₂ (450.00 mg, 3.783 mmol, 1.5 equiv) in portions at 0° C. The resulting mixture was stirred for overnight at room temperature. The reaction was monitored by TLC. The mixture was acidified with 1N HCl. The resulting mixture was extracted with DCM. The combined organic layers were washed with brine, dried over anhydrous Na₂SO₄. After filtration, the filtrate was concentrated under reduced pressure to afford 19b (600 mg, 80.17%) as a white solid.

### Step B. Tert-butyl 6-{4-[7′-(2-methylcyclopentyl)-6′-oxospiro[cyclopropane-1,5′-pyrrolo[2,3-d]pyrimidin]-2′-ylamino]piperidin-1-ylsulfonyl}-3,6-diazabicyclo[3.2.0]heptane-3-carboxylate (19c)

To a solution of Int-2 (200 mg, 0.586 mmol, 1 equiv) and Et₃N (1778.13 mg, 17.580 mmol, 30 equiv) in THF (4 mL) was added 19b (347.65 mg, 1.172 mmol, 2 equiv) at room temperature. The resulting mixture was stirred for 2 h at room temperature. Desired product could be detected by LCMS. The resulting mixture was concentrated under reduced pressure. The residue was purified by silica gel column chromatography, eluted with CH₂Cl₂/MeOH (30:1) to afford 19c (130 mg, 28.58%) as a yellow solid. LCMS (ESI, m/z): [M+H]⁺ 602.3.

### Step C. 2′-[(1-{3,6-Diazabicyclo[3.2.0]heptane-6-sulfonyl}piperidin-4-yl)amino]-7′-(2-methylcyclopentyl)spiro [cyclopropane-1,5′-pyrrolo[2,3-d]pyrimidin]-6′-one (Int-19)

To a stirred solution of 19c (50 mg, 0.083 mmol, 1 equiv) in CH₃CN (2.5 mL) was added 4M HCl (gas) in 1,4-dioxane (0.5 mL) dropwise at 0° C. The resulting mixture was stirred for 30 min at 0° C. Desired product could be detected by LCMS. The resulting mixture was concentrated under reduced pressure. The mixture was basified to pH 7 with Et₃N. The resulting mixture was concentrated under reduced pressure. The crude product was purified by Prep-HPLC with the following conditions (Column: XBridge Prep OBD C18 Column, 30*150 mm, 10 μm; Mobile Phase A: water (10 mmol/L NH₄HCO₃+0.05% NH₃·H₂O), Mobile Phase B: ACN; Flow rate: 60 mL/min mL/min; Gradient: 32% B to 42% B in 8 min; Wave Length: 254 nm/220 nm nm; RT1 (min): 9.3) to afford Int-19 (8.4 mg, 19.75%) as a white solid. LCMS (ESI, m/z): [M+H]⁺ 502.2.

### 2′-((1-((2,7-Diazaspiro[4.4]nonan-2-yl)sulfonyl)piperidin-4-yl)amino)-7′-(2-methylcyclopentyl)spiro [cyclopropane-1,5′-pyrrolo[2,3-d]pyrimidin]-6′(7′H)-one (Int-20)

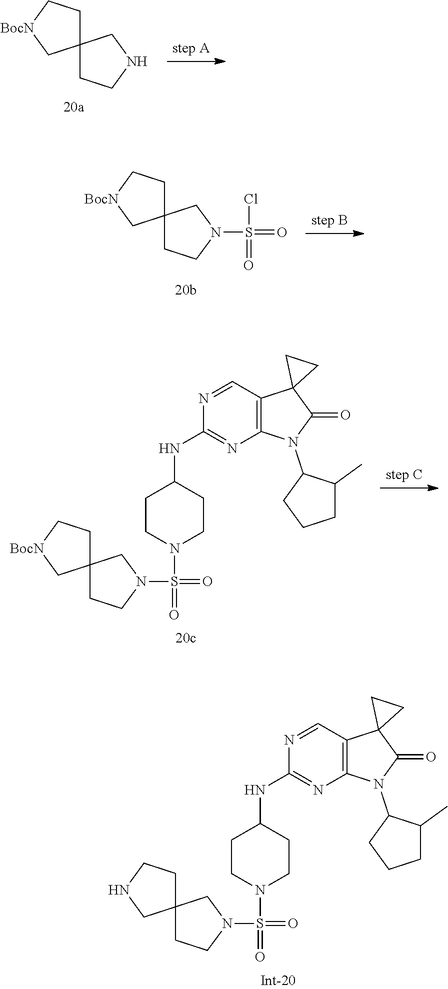 [SMILES: CC(C)(C)OC(=O)N1CCC2(CCNC2)C1 ; CC(C)(C)OC(=O)N1CCC2(CCN(S(=O)(=O)Cl)C2)C1 ; CC1CCCC1N1C(=O)C2(CC2)c2cnc(NC3CCN(S(=O)(=O)N4CCC5(CCN(C(=O)OC(C)(C)C)C5)C4)CC3)nc21 ; CC1CCCC1N1C(=O)C2(CC2)c2cnc(NC3CCN(S(=O)(=O)N4CCC5(CCNC5)C4)CC3)nc21]

### Step A. Tert-butyl 7-(chlorosulfonyl)-2,7-diazaspiro[4.4]nonane-2-carboxylate (20b)

To a stirred solution of SO₂Cl₂ (143.12 mg, 1.061 mmol, 1.2 equiv) in DCM (5 mL) was added the solution of 20a (200 mg, 0.884 mmol, 1 equiv) and pyridine (104.85 mg, 1.326 mmol, 1.5 equiv) in DCM (2 mL) dropwise at 0° C. The resulting mixture was stirred for overnight at room temperature. Desired product could be detected by LCMS. The mixture was acidified with 1N HCl. The resulting mixture was extracted with DCM. The combined organic layers were washed with brine, dried over anhydrous Na₂SO₄. After filtration, the filtrate was concentrated under reduced pressure to afford 20b (250 mg, 87.09%) as an off-white solid.

### Step B. Tert-butyl 7-{4-[7′-(2-methylcyclopentyl)-6′-oxospiro[cyclopropane-1,5′-pyrrolo[2,3-d]pyrimidin]-2′-ylamino]piperidin-1-ylsulfonyl}-2,7-diazaspiro[4.4]nonane-2-carboxylate (20c)

To a solution of Int-2 (300 mg, 0.879 mmol, 1 equiv) and TEA (2.67 g, 26.370 mmol, 30 equiv) in THF (5 mL) was added 20b (428.07 mg, 1.319 mmol, 1.5 equiv) at 0° C. The resulting mixture was stirred for 2 h at room temperature under nitrogen atmosphere. The resulting mixture was diluted with water (5 mL). The resulting mixture was extracted with EtOAc (3×15 mL). The combined organic layers were dried over anhydrous Na₂SO₄. After filtration, the filtrate was concentrated under reduced pressure. The residue was purified by silica gel column chromatography, eluted with petroleum ether/EtOAc (1:4) to afford 20c (320 mg, 53.09%) as a white solid. LCMS (ESI, m/z): [M+H]⁺ 630.3.

### Step C. 2′-((1-((2,7-Diazaspiro[4.4]nonan-2-yl)sulfonyl)piperidin-4-yl)amino)-7′-(2-methylcyclopentyl) spiro [cyclopropane-1,5′-pyrrolo[2,3-d]pyrimidin]-6′(7′H)-one (Int-20)

To a stirred solution of 20c (60 mg, 0.095 mmol, 1 equiv) in DCM (1 mL) was added CF₃COOH (0.2 mL) dropwise at 0° C. The resulting mixture was stirred for 1 h at 0° C. The resulting mixture was concentrated under reduced pressure. The mixture was neutralized to pH 7 with Et₃N. The residue was purified by reversed-phase flash chromatography to afford Int-20 (12.5 mg, 24.30%) as a white solid.

### 2′-f[1-(1,4-Diazepane-1-sulfonyl)piperidin-4-yl]amino}-7′-(2-methylcyclopentyl)spiro[cyclopropane-1,5′-pyrrolo[2,3-d]pyrimidin]-6′-one (Int-21)

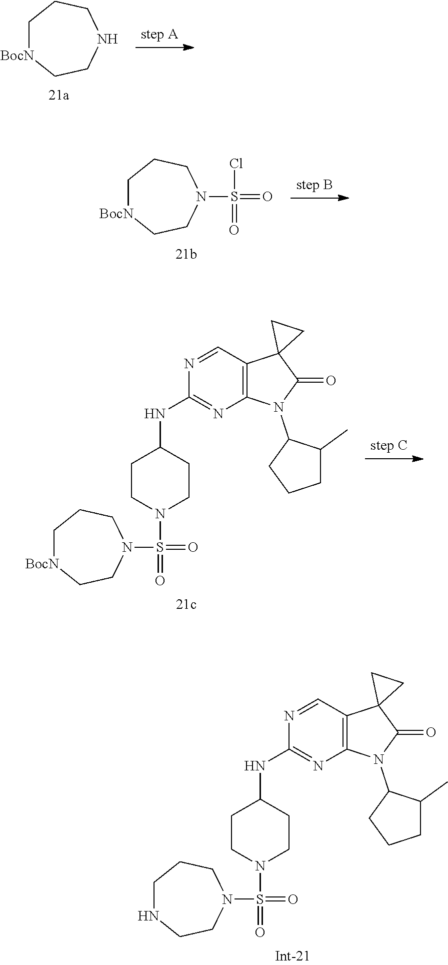 [SMILES: CC(C)(C)OC(=O)N1CCCNCC1 ; CC(C)(C)OC(=O)N1CCCN(S(=O)(=O)Cl)CC1 ; CC1CCCC1N1C(=O)C2(CC2)c2cnc(NC3CCN(S(=O)(=O)N4CCCN(C(=O)OC(C)(C)C)CC4)CC3)nc21 ; CC1CCCC1N1C(=O)C2(CC2)c2cnc(NC3CCN(S(=O)(=O)N4CCCNCC4)CC3)nc21]

### Step A. Tert-butyl 4-(chlorosulfonyl)-1,4-diazepane-1-carboxylate (21b)

To a stirred solution of SO₂Cl₂ (242.59 mg, 1.798 mmol, 1.2 equiv) in DCM (5 mL) was added the solution of 21a (300 mg, 1.498 mmol, 1 equiv) and pyridine (177.72 mg, 2.247 mmol, 1.5 equiv) in DCM (2 mL) dropwise at 0° C. The resulting mixture was stirred for overnight at room temperature. Desired product could be detected by LCMS. The mixture was acidified with 1N HCl. The resulting mixture was extracted with DCM. The combined organic layers were washed with brine, dried over anhydrous Na₂SO₄. After filtration, the filtrate was concentrated under reduced pressure to afford 21b (400 mg, 89.38%) as a brown solid. The crude product was used in the next step directly without further purification.

### Step B. Tert-butyl 4-{4-[7′-(2-methylcyclopentyl)-6′-oxospiro[cyclopropane-1,5′-pyrrolo[2,3-d]pyrimidin]-2′-ylamino]piperidin-1-ylsulfonyl}-1,4-diazepane-1-carboxylate (21c)

To a solution of Int-2 (100 mg, 0.293 mmol, 1 equiv) and Et₃N (88.86 mg, 0.879 mmol, 3 equiv) in THF (2 mL) was added 21b (175.45 mg, 0.586 mmol, 2 equiv) at room temperature. The resulting mixture was stirred for 2 h at room temperature. Desired product could be detected by LCMS. The resulting mixture was concentrated under reduced pressure. The residue was purified by silica gel column chromatography, eluted with CH₂Cl₂/MeOH (30:1) to afford 21c (50 mg, 28.28%) as a yellow solid. LCMS (ESI, m/z): [M+H]⁺ 604.3.

### Step C. 2′-{[1-(1,4-Diazepane-1-sulfonyl)piperidin-4-yl]amino}-7′-(2-methylcyclopentyl)spiro [cyclopropane-1,5′-pyrrolo[2,3-d]pyrimidin]-6′-one (Int-21)

To a stirred solution of 21c (50 mg, 0.083 mmol, 1 equiv) in CH₃CN (2.5 mL) were added 4M HCl (gas) in 1,4-dioxane (0.5 mL) dropwise at 0° C. The resulting mixture was stirred for 30 min at 0° C. The mixture was basified to pH 7 with Et₃N. The resulting mixture was concentrated under reduced pressure. The crude product was purified by Prep-HPLC with the following conditions (Column: YMC-Actus Triart C18 EXRS 30*150 mm, 5 μm; Mobile Phase A: Water (10 mmol/L NH₄HCO₃+0.05% NH₃·H₂O), Mobile Phase B: ACN; Flow rate: 60 mL/min mL/min; Gradient: 37% B to 53% B in 8 min; Wave Length: 254 nm/220 nm nm; RT1 (min): 7.45) to afford Int-21 (3.6 mg, 8.49%) as a white solid. LCMS (ESI, m/z): [M+H]⁺ 504.3.

### 7′-(2-methylcyclopentyl)-2′-((1-(piperidin-3-ylsulfonyl)piperidin-4-yl)amino)spiro[cyclopropane-1,5′-pyrrolo[2,3-d]pyrimidin]-6′(7′H)-one (Int-22)

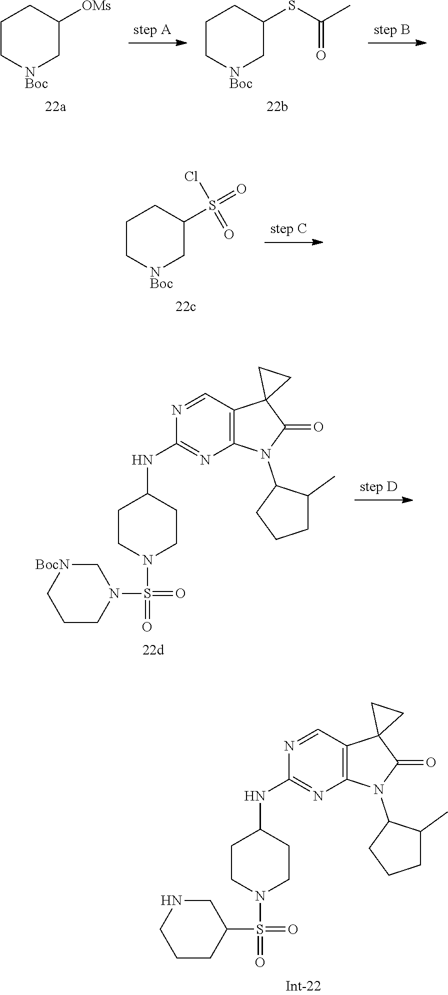 [SMILES: CC(C)(C)OC(=O)N1CCCC(OS(C)(=O)=O)C1 ; CC(=O)SC1CCCN(C(=O)OC(C)(C)C)C1 ; CC(C)(C)OC(=O)N1CCCC(S(=O)(=O)Cl)C1 ; CC1CCCC1N1C(=O)C2(CC2)c2cnc(NC3CCN(S(=O)(=O)N4CCCN(C(=O)OC(C)(C)C)C4)CC3)nc21 ; CC1CCCC1N1C(=O)C2(CC2)c2cnc(NC3CCN(S(=O)(=O)C4CCCNC4)CC3)nc21]

### Step A. tert-butyl 3-(acetylsulfanyl) piperidine-1-carboxylate (22b)

A solution of 22a (16.19 g, 141.757 mmol, 1.2 equiv) in DMF (400 mL) was stirred for overnight at 80° C. under nitrogen atmosphere. Desired product could be detected by LCMS. The resulting mixture was diluted with brine (500 mL). The resulting mixture was extracted with EtOAc (1 L). The combined organic layers were washed with brine (50×10 mL), dried over anhydrous Na₂SO₄. After filtration, the filtrate was concentrated under reduced pressure. The residue was purified by silica gel column chromatography, eluted with petroleum ether/EtOAc (1:1) to afford 22b (10 g, 29.37%) as a pink solid. LCMS (ESI, m/z): [M+H]260.1.

### Step B. tert-butyl 3-(chlorosulfonyl) piperidine-1-carboxylate (22c)

To a stirred mixture of 22b (1 g, 3.856 mmol, 1 equiv) in HOAc (100 mL) and H₂O (10 mL) was added NCS (1.03 g, 7.712 mmol, 2 equiv) at room temperature under nitrogen atmosphere. The resulting mixture was stirred for overnight at room temperature under nitrogen atmosphere. Desired product could be detected by LCMS. The resulting mixture was concentrated under reduced pressure. The resulting mixture was diluted with water (200 mL). The resulting mixture was extracted with CH₂Cl₂ (500 mL). The combined organic layers were washed with brine (20×10 mL), dried over anhydrous Na₂SO₄. After filtration, the filtrate was concentrated under reduced pressure. The crude product was used in the next step directly without further purification. This resulted in 22c (1 g, crude) as a light yellow oil. LCMS (ESI, m/z): [M+H]⁺ 284.1.

### Step C. tert-butyl 3-{4-[7′-(2-methylcyclopentyl)-6′-oxospiro[cyclopropane-1,5′-pyrrolo[2,3-d]pyrimidin]-2′-ylamino]piperidin-1-ylsulfonyl}piperidine-1-carboxylate (22d)

To a stirred mixture of 22c (900 mg, 3.172 mmol, 1 equiv) and Int-2 (1624.45 mg, 4.758 mmol, 1.5 equiv) in THF (10 mL) was added TEA (9628.26 mg, 95.160 mmol, 30 equiv) at room temperature under nitrogen atmosphere. The resulting mixture was stirred for 2 h at room temperature under nitrogen atmosphere. Desired product could be detected by LCMS. The resulting mixture was concentrated under reduced pressure. The residue was purified by silica gel column chromatography, eluted with petroleum ether/EtOAc (1:2) to afford 22d (800 mg, 38.56%) as an off-white solid. LCMS (ESI, m/z): [M+H]⁺ 589.3.

### Step D. 7′-(2-methylcyclopentyl)-2′-{[1-(piperidine-3-sulfonyl)piperidin-4-yl]amino}spiro [cyclopropane-1,5′-pyrrolo[2,3-d]pyrimidin]-6′-one (Int-22)

A solution of 22d (50 mg, 0.085 mmol, 1 equiv) and HCl (gas) in 1,4-dioxane (0.5 mL, 16.456 mmol, 193.78 equiv) in MeCN (2.5 mL) was stirred for 2 h at 0° C. under air atmosphere. Desired product could be detected by LCMS. The resulting mixture was concentrated under reduced pressure. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. This resulted in Int-22 (16.6 mg, 39.68%) as a white solid. LCMS (ESI, m/z): [M+H]⁺ 489.3.

### 2′-((4-((3-(azetidin-3-yloxy)phenyl)sulfonyl)phenyl)amino)-7′-((1R,3R)-3-hydroxycyclohexyl)spiro [cyclopropane-1,5′-pyrrolo[2,3-d]pyrimidin]-6′(7′H)-one (Int-23)

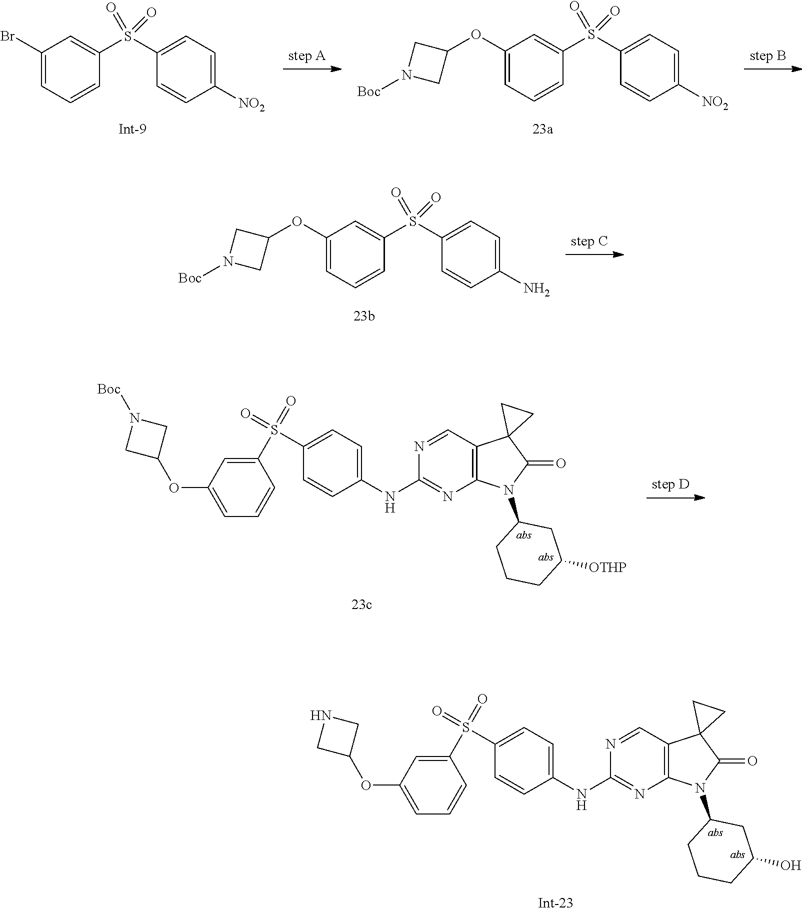

### Step A. tert-butyl 3-(3-((4-nitrophenyl)sulfonyl)phenoxy)azetidine-1-carboxylate (23a)

To a stirred solution of Int-9 (2 g, 5.845 mmol, 1 equiv) and tert-butyl 3-hydroxyazetidine-1-carboxylate (1.11 g, 6.429 mmol, 1.1 equiv) in 1,2-dioxane (30 mL) were added EPhos Pd G4 (0.54 g, 0.585 mmol, 0.1 equiv) and Cs₂CO₃ (5.71 g, 17.535 mmol, 3 equiv) in portions at 25° C. under nitrogen atmosphere. The resulting mixture was stirred for 2 h at 90° C. under nitrogen atmosphere. Desired product could be detected by LCMS. The resulting mixture was extracted with EtOAc (3×15 mL). The combined organic layers were washed with brine (3×10 mL), dried over anhydrous Na₂SO₄. The resulting mixture was concentrated under reduced pressure. The residue was purified by silica gel column chromatography, eluted with petroleum ether/EtOAc (5:1) to afford 23a (650 mg, 25.60%) as a yellow solid. LCMS (ESI, m/z): [M−56+H]⁺ 376.0.

### Step B. tert-butyl 3-[3-(4-aminobenzenesulfonyl)phenoxy]azetidine-1-carboxylate (23b)

To a solution of 23a (650 mg, 1.496 mmol, 1 equiv) in 15 mL CF₃CH₂OH was added Pd/C (10%, 550 mg) under nitrogen atmosphere in a 50 mL round-bottom flask. The mixture was hydrogenated at room temperature for 2 h under hydrogen atmosphere using a hydrogen balloon. Desired product could be detected by LCMS. The resulting mixture was filtered through a Celite pad and concentrated under reduced pressure to afford 23b (600 mg, crude) as a yellow solid. LCMS (ESI, m/z): [M+H−100]⁺ 305.0

### Step C. tert-butyl 3-(3-((4-((6′-oxo-7′-((1R,3R)-3-((tetrahydro-2H-pyran-2-yl)oxy)cyclohexyl)-6′,7′-dihydrospiro[cyclopropane-1,5′-pyrrolo[2,3-d]pyrimidin]-2′-yl)amino)phenyl)sulfonyl)phenoxy) azetidine-1-carboxylate (23c)

To a stirred solution of tert-butyl 23b (600 mg, 1.483 mmol, 1 equiv) and Int-5 (672.63 mg, 1.780 mmol, 1.2 equiv) in dioxane (8 mL) were added RuPhos Pd G3 (186.24 mg, 0.222 mmol, 0.15 equiv) and Cs₂CO₃ (1449.95 mg, 4.449 mmol, 3 equiv) in portions at room temperature. The resulting mixture was stirred for 1 h at 90° C. under nitrogen atmosphere. Desired product could be detected by LCMS. The resulting mixture was extracted with EtOAc (3×10 mL). The combined organic layers were washed with brine (3×10 mL), dried over anhydrous Na₂SO₄. After filtration, the filtrate was concentrated under reduced pressure. The resulting mixture was concentrated under reduced pressure. The residue was purified by silica gel column chromatography, eluted with petroleum ether/EtOAc (1:1) to afford 23c (500 mg, 42.39%) as a yellow solid. LCMS (ESI, m/z): [M+H]⁺ 746.2.

### Step D. 2′-({4-[3-(azetidin-3-yloxy)benzenesulfonyl]phenyl}amino)-7′-[(1R,3R)-3-hydroxycyclohexyl]spiro[cyclopropane-1,5′-pyrrolo[2,3-d]pyrimidin]-6′-one (Int-23)

To a stirred solution of 23c (500 mg, 0.670 mmol, 1 equiv) in MeCN (5 mL) was added HCl (gas) in 1,4-dioxane (5 mL) dropwise at 0° C. under Hydrogen chloride atmosphere. The resulting mixture was stirred for 1 h at 0° C. under Hydrogen chloride atmosphere. Desired product could be detected by LCMS. The resulting mixture was concentrated under reduced pressure to afford Int-23 (230 mg, 61.09%) as a white solid. LCMS (ESI, m/z): [M+H]⁺ 562.1

### 1-[1-methyl-6-(piperidin-4-yl) indazol-3-yl]-1,3-diazinane-2,4-dione (Int-24)

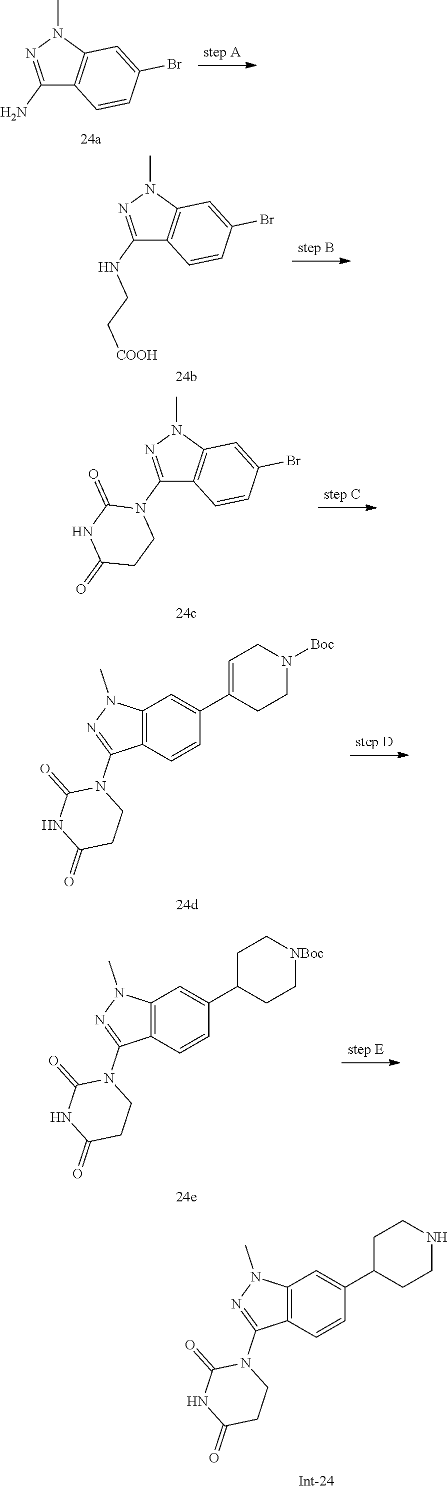 [SMILES: Cn1nc(N)c2ccc(Br)cc21 ; Cn1nc(NCCC(=O)O)c2ccc(Br)cc21 ; Cn1nc(N2CCC(=O)NC2=O)c2ccc(Br)cc21 ; Cn1nc(N2CCC(=O)NC2=O)c2ccc(C3=CCN(C(=O)OC(C)(C)C)CC3)cc21 ; Cn1nc(N2CCC(=O)NC2=O)c2ccc(C3CCN(C(=O)OC(C)(C)C)CC3)cc21 ; Cn1nc(N2CCC(=O)NC2=O)c2ccc(C3CCNCC3)cc21]

### Step A. 3-[(6-Bromo-1-methylindazol-3-yl)amino]propanoic acid (24b)

To a stirred solution of 24a (5 g, 22.116 mmol, 1 equiv) and HOAc (3.15 g, 52.415 mmol, 2.37 equiv) in H₂O (8 mL) was added acrylic acid (1.59 g, 22.116 mmol, 1 equiv) dropwise at room temperature. The resulting mixture was stirred for overnight at 100° C. under nitrogen atmosphere. Desired product could be detected by LCMS. The mixture was basified to pH 7 with 6N HCl (aq.). The aqueous layer was extracted with EtOAc (3×50 mL). The combined organic layers were dried over anhydrous Na₂SO₄. After filtration, the filtrate was concentrated under reduced pressure. The residue was purified by silica gel column chromatography, eluted with CH₂Cl₂/MeOH (10:1) to afford 24b (1.84 g, 25.64%) as a yellow solid. LCMS (ESI, m/z): [M+H]⁺=298.0

### Step B. 1-(6-Bromo-1-methylindazol-3-yl)-1,3-diazinane-2,4-dione (24c)

To a stirred solution of 24b (900 mg, 3.019 mmol, 1 equiv) in HOAc (9 mL) was added urea (906.46 mg, 15.095 mmol, 5 equiv) in portions at room temperature under nitrogen atmosphere. The resulting mixture was stirred for overnight at 120° C. under nitrogen atmosphere. Desired product could be detected by LCMS. The resulting mixture was concentrated under reduced pressure. The residue was purified by silica gel column chromatography, eluted with CH₂Cl₂/MeOH (10:1) to afford 24c (330 mg, 32.44%) as a yellow solid. LCMS (ESI, m/z): [M+H]⁺ 323.0

### Step C. Tert-butyl 4-[3-(2,4-dioxo-1,3-diazinan-1-yl)-1-methylindazol-6-yl]-3,6-dihydro-2H-pyridine-1-carboxylate (24d)

To a solution of 24c (310 mg, 0.959 mmol, 1 equiv) and tert-butyl 4-(4,4,5,5-tetramethyl-1,3,2-dioxaborolan-2-yl)-3,6-dihydro-2H-pyridine-1-carboxylate (444.94 mg, 1.438 mmol, 1.5 equiv) in dioxane (4 mL) and H₂O (0.4 mL) were added Xphos Pd G3 (81.20 mg, 0.096 mmol, 0.1 equiv) and K₃PO₄ (610.88 mg, 2.877 mmol, 3 equiv). After stirring for 3 h at 60° C. under a nitrogen atmosphere, the resulting mixture was concentrated under reduced pressure. The resulting mixture was concentrated under reduced pressure. The residue was purified by silica gel column chromatography, eluted with petroleum ether/EtOAc (1:1) to afford 24d (320 mg, 53.62%) as a white solid. LCMS (ESI, m/z): [M+H]⁺ 426.2

### Step D. Tert-butyl 4-[3-(2,4-dioxo-1,3-diazinan-1-yl)-1-methylindazol-6-yl]piperidine-1-carboxylate (24e)

To a solution of 24d (320 mg, 0.752 mmol, 1 equiv) in CF₃CH₂OH (20 mL) was added Pd/C (160.07 mg, 1.504 mmol, 2 equiv) in a pressure tank. The mixture was hydrogenated at room temperature under 30 psi of hydrogen pressure for 3 h, filtered through a Celite pad and concentrated under reduced pressure. The residue was purified by silica gel column chromatography, eluted with petroleum ether/EtOAc (1:1) to afford 24e (240 mg, 71.06%) as a white solid. LCMS (ESI, m/z): [M+H]⁺ 428.2

### Step E. 1-[1-Methyl-6-(piperidin-4-yl) indazol-3-yl]-1,3-diazinane-2,4-dione (Int-24)

To a stirred solution of 24e (50 mg, 0.117 mmol, 1 equiv) in CH₃CN (2.5 mL) was added 4M HCl (gas) in 1,4-dioxane (0.5 mL) dropwise at 0° C. The resulting mixture was stirred for 30 min at 0° C. Desired product could be detected by LCMS. The mixture was basified to pH 7 with saturated NaHCO₃ (aq.). The aqueous layer was extracted with EtOAc (3×30 mL). The combined organic layers were dried over anhydrous Na₂SO₄. After filtration, the filtrate was concentrated under reduced pressure. The crude product of Int-24 was used in the next step directly without further purification. LCMS (ESI, m/z): [M+H]⁺ 328.1

### 3-(1-Oxo-5-(2,6-diazaspiro[3.3]heptan-2-yl)isoindolin-2-yl)piperidine-2,6-dione (Int-25)

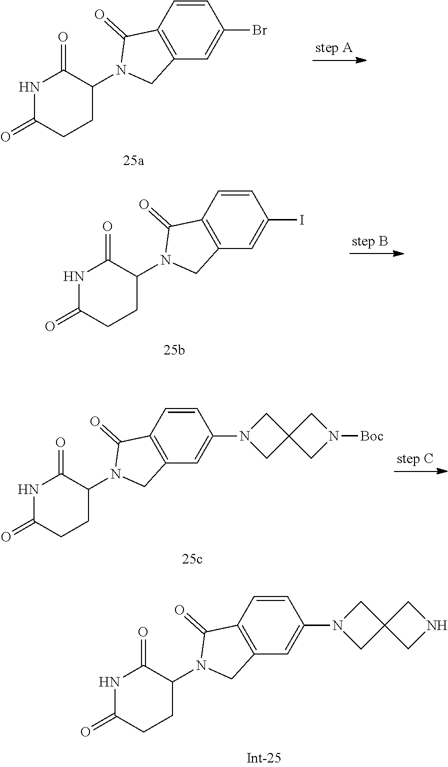 [SMILES: O=C1CCC(N2Cc3cc(Br)ccc3C2=O)C(=O)N1 ; O=C1CCC(N2Cc3cc(I)ccc3C2=O)C(=O)N1 ; CC(C)(C)OC(=O)N1CC2(C1)CN(c1ccc3c(c1)CN(C1CCC(=O)NC1=O)C3=O)C2 ; O=C1CCC(N2Cc3cc(N4CC5(CNC5)C4)ccc3C2=O)C(=O)N1]

### Step A. 3-(5-Iodo-1-oxo-3H-isoindol-2-yl) piperidine-2,6-dione (Int-25)

To a solution of 25a (11 g, 34.040 mmol, 1 equiv) in 1,4-dioxane (110 mL) was added (1R,2R)—N1,N2-dimethylcyclohexane-1,2-diamine (1.94 g, 13.616 mmol, 0.4 equiv), NaI (20.424 g, 136.160 mmol, 4 equiv), CuI (2.59 g, 13.616 mmol, 0.4 equiv) at room temperature. The mixture was stirred for overnight at 125° C. Desired product could be detected by LCMS. The resulting mixture was concentrated under reduced pressure. The residue was purified by silica gel column chromatography, eluted with CH₂Cl₂/MeOH (10/1) to afford 25b (9.1 g, 72.22%) as a dark green solid. LCMS (ESI, m/z): [M+H]⁺ 371.0.

### Step B. Tert-butyl 6-(2-(2,6-dioxopiperidin-3-yl)-1-oxoisoindolin-5-yl)-2,6-diazaspiro[3.3]heptane-2-carboxylate (25c)

To a mixture of 25b (200 mg, 0.540 mmol, 1 equiv) and tert-butyl 2,6-diazaspiro[3.3]heptane-2-carboxylate (107.13 mg, 0.540 mmol, 1 equiv) and Pd-PEPPSI-IPentCl 3-chloropyridine (39.35 mg, 0.054 mmol, 0.1 equiv) in dioxane (10 mL) were added Cs₂CO₃ (352.10 mg, 1.080 mmol, 2 equiv) at 25° C. under N2. The resulting mixture was stirred for 1 h at 80° C. Desired product could be detected by LCMS. The mixture was concentrated under vacuum. The resulting mixture was diluted with water (5 mL) and extracted with EtOAc (3×10 mL). The combined organic layers were dried over anhydrous Na₂SO₄ and concentrated under reduced pressure. The residue was purified by reversed-phase Combi-Flash, eluted with CH₃CN:H₂O (0.5% NH₄HCO₃)=5%-60%. This resulted in 25c (100 mg, 35.71%) as a yellow solid. LCMS (ESI, m/z): [M+H]⁺ 441.2.

### Step C. 3-(1-Oxo-5-(2,6-diazaspiro[3.3]heptan-2-yl)isoindolin-2-yl)piperidine-2,6-dione (Int-25)

To a stirred solution of 25c (100 mg, 0.227 mmol, 1 equiv) in CH₂Cl₂ (10 mL) was added TFA (776.55 mg, 6.810 mmol, 30 equiv) at 0° C. under N₂. The resulting mixture was stirred for 1 h at 25° C. Desired product could be detected by LCMS. The mixture was concentrated under vacuum. The resulting mixture was diluted with water (5 mL) and extracted with EtOAc (3×10 mL). The combined organic layers were dried over anhydrous Na₂SO₄ and concentrated under reduced pressure. The residue was purified by reversed-phase Combi-Flash to afford Int-25 (65 mg, 63.09%) as a yellow solid. LCMS (ESI, m/z): [M+H]⁺ 341.2.

### 3-[6-Fluoro-1-oxo-5-(piperazin-1-yl)-3H-isoindol-2-yl]piperidine-2,6-dione (Int-26)

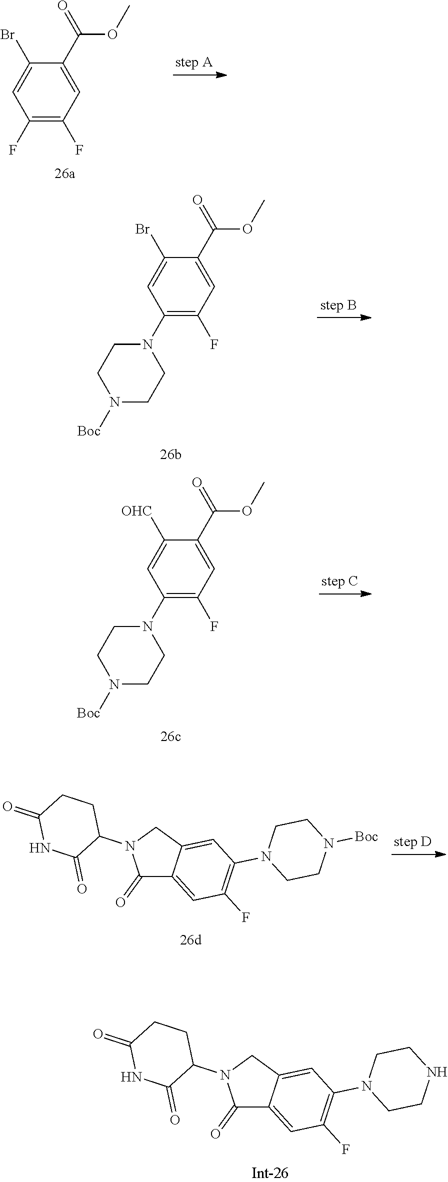 [SMILES: COC(=O)c1cc(F)c(F)cc1Br ; COC(=O)c1cc(F)c(N2CCN(C(=O)OC(C)(C)C)CC2)cc1Br ; COC(=O)c1cc(F)c(N2CCN(C(=O)OC(C)(C)C)CC2)cc1C=O ; CC(C)(C)OC(=O)N1CCN(c2cc3c(cc2F)C(=O)N(C2CCC(=O)NC2=O)C3)CC1 ; O=C1CCC(N2Cc3cc(N4CCNCC4)c(F)cc3C2=O)C(=O)N1]

### Step A. Tert-butyl 4-(5-bromo-2-fluoro-4-(methoxycarbonyl)phenyl)piperazine-1-carboxylate (26b)

To a stirred mixture of 26a (2 g, 7.967 mmol, 1 equiv) and tert-butyl piperazine-1-carboxylate (1.48 g, 7.967 mmol, 1 equiv) in DMF (10 mL) were added K₂CO₃ (3.30 g, 23.901 mmol, 3 equiv) in portions at room temperature under nitrogen atmosphere. The resulting mixture was stirred for overnight at 80° C. under nitrogen atmosphere. Desired product could be detected by LCMS. The resulting mixture was diluted with water (20 mL). The resulting mixture was extracted with EtOAc (3×20 mL). The combined organic layers were washed with water (3×10 mL), dried over anhydrous Na₂SO₄. After filtration, the filtrate was concentrated under reduced pressure. The residue was purified by silica gel column chromatography, eluted with petroleum ether/EtOAc (6:1) to afford 26b (2.3 g, 69.18%) as a brown solid. LCMS (ESI, m/z): [M+H]⁺ 417.1.

### Step B. Tert-butyl 4-[2-fluoro-5-formyl-4-(methoxycarbonyl)phenyl]piperazine-1-carboxylate (26c)

To a stirred mixture of 26b (2.3 g, 5.512 mmol, 1 equiv) and 2-isocyano-2-methylpropane (0.92 g, 11.024 mmol, 2 equiv) in DMF (20 mL) were added Palladium acetate (0.12 g, 0.551 mmol, 0.1 equiv) and triethylsilane (0.06 g, 0.551 mmol, 0.1 equiv) and tricyclohexylphosphane (4.64 g, 16.536 mmol, 3 equiv) and Na2CO3 (1.75 g, 16.536 mmol, 3 equiv) room temperature under nitrogen atmosphere. The resulting mixture was stirred for overnight at 35° C. under nitrogen atmosphere. Desired product could be detected by LCMS. The resulting mixture was diluted with water (20 mL). The resulting mixture was extracted with EtOAc (3×20 mL). The combined organic layers were washed with water (3×10 mL), dried over anhydrous Na₂SO₄. After filtration, the filtrate was concentrated under reduced pressure. The residue was purified by silica gel column chromatography, eluted with petroleum ether/EtOAc (5:1) to afford 26c (1.3 g, 64.37%) as a brown solid. LCMS (ESI, m/z): [M+H]⁺ 367.2.

### Step C. Tert-butyl 4-(2-(2,6-dioxopiperidin-3-yl)-6-fluoro-1-oxoisoindolin-5-yl)piperazine-1-carboxylate (26d)

To a stirred mixture of 3-aminopiperidine-2,6-dione (0.68 g, 5.322 mmol, 1.5 equiv) and 26c (1.3 g, 3.548 mmol, 1 equiv) in MeOH (10 mL) were added HOAc (0.64 g, 10.657 mmol, 3.00 equiv) at room temperature under air atmosphere. The resulting mixture was stirred for 0.5 h at room temperature under nitrogen atmosphere. To the above mixture was added NaBH₃CN (0.33 g, 5.322 mmol, 1.5 equiv) in portions over 2 min at room temperature. The resulting mixture was stirred for additional overnight at room temperature. Desired product could be detected by LCMS. The residue was purified by silica gel column chromatography, eluted with petroleum ether/EtOAc (2:1) to afford 26d (1.1 g, 69.44%) as an off-white solid. LCMS (ESI, m/z): [M+H]⁺ 447.2.

### Step D. 3-[6-Fluoro-1-oxo-5-(piperazin-1-yl)-3H-isoindol-2-yl]piperidine-2,6-dione (Int-26)

To a stirred solution of 26d (60 mg, 0.134 mmol, 1 equiv) in ACN (1.5 mL) was added 4 M HCl (gas) in 1,4-dioxane (0.3 mL) dropwise at 0° C. under nitrogen atmosphere. The resulting mixture was stirred for 1 h at 0° C. Desired product could be detected by LCMS. The resulting mixture was concentrated under reduced pressure. This resulted in Int-26 (50 mg, crude) as a white solid. The crude product was used in the next step directly without further purification. LCMS (ESI, m/z): [M+H]⁺ 347.1.

### 3-[5-(Azetidin-3-yl)-1-oxo-3H-isoindol-2-yl]piperidine-2,6-dione (Int-27)

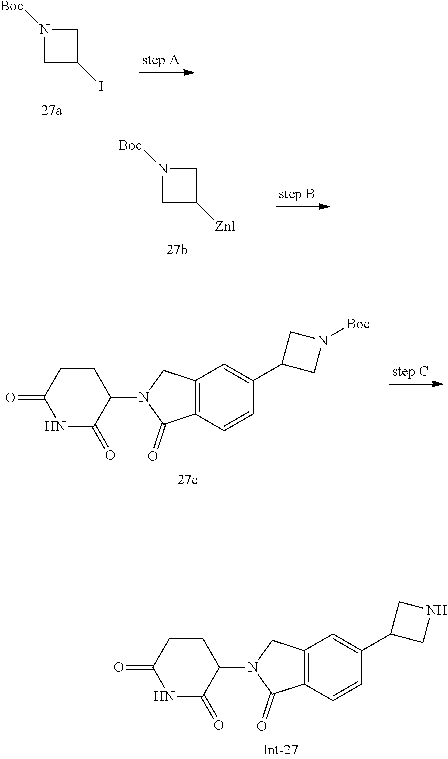

### Step A. (1-(Tert-butoxycarbonyl)azetidin-3-yl)zinc(II) iodide (27b)

To a stirred solution of 27a (2 g, 7.064 mmol, 1 equiv) and Zn (923.76 mg, 14.128 mmol, 2 equiv) in THF (60 mL) were added TMSCl (307 mg, 2.824 mmol, 0.4 equiv) and BrCH₂CH₂Br (525.6 mg, 2.824 mmol, 0.4 equiv) in portions at room temperature. The resulting mixture was stirred for 2 h at 60° C. under nitrogen atmosphere. Desired product could be detected by TLC (petroleum ether/EtOAc=1:1, Rf=0.5). The resulting mixture was used in the next step directly without further purification.

### Step B. Tert-butyl 3-[2-(2,6-dioxopiperidin-3-yl)-1-oxo-3H-isoindol-5-yl]azetidine-1-carboxylate (27c)

To the above mixture were added 27b (400 mg, 1.08 mmol, 1.00 equiv), Pd(dppf)Cl₂ (79.08 mg, 0.108 mmol, 0.1 equiv) and CuI (20.58 mg, 0.108 mmol, 0.1 equiv) at room temperature. The resulting mixture was stirred for 1 h at 80° C. under nitrogen atmosphere. Desired product could be detected by LCMS. The resulting mixture was extracted with EtOAc (3×10 mL). The combined organic layers were washed with brine (10 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in water (0.1% FA), 10% to 50% gradient in 30 min. The resulting mixture was concentrated under reduced pressure to afford 27c (120 mg, 27.80%) as a brown solid. LCMS (ESI, m/z): [M+H]⁺ 400.2.

### Step C. 3-[5-(Azetidin-3-yl)-1-oxo-3H-isoindol-2-yl]piperidine-2,6-dione (Int-27)

To a stirred solution of 27c (150 mg, 0.336 mmol, 1 equiv) in anhydrous MeCN (2.5 mL) was added 4M HCl (gas) in 1,4-dioxane (0.5 mL) at 0° C. and stirred for 1 h. Desired product could be detected by LCMS. The resulting mixture was concentrated under reduced pressure. The resulting mixture was used in the next step directly without further purification. This resulted in Int-27 (150 mg, crude) as a white solid. The crude product was used in the next step directly without further purification. LCMS (ESI, m/z): [M+H]⁺ 300.1

### 1-[2-(2,6-Dioxopiperidin-3-yl)-1-oxo-3H-isoindol-5-yl]piperidine-4-carbaldehyde (Int-28}

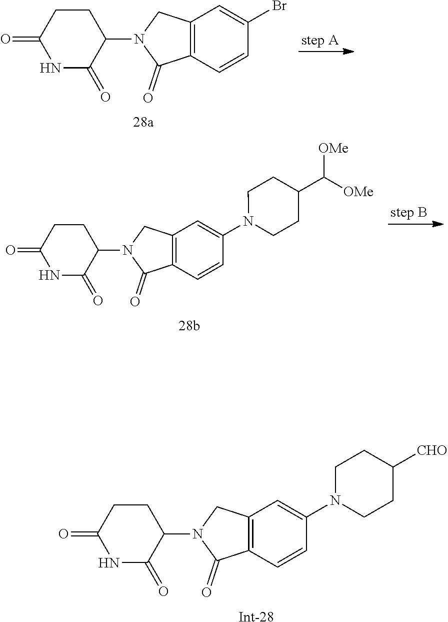

### Step A. 3-{5-[4-(Dimethoxymethyl)piperidin-1-yl]-1-oxo-3H-isoindol-2-yl}piperidine-2,6-dione (28b)

To a mixture of 28a (1 g, 3.095 mmol, 1 equiv) and 4-(dimethoxymethyl)piperidine (1.23 g, 7.738 mmol, 2.5 equiv) in DMSO (10 mL) was added Pd-PEPPSI-IPentCl 3-chloropyridine (245.30 mg, 0.310 mmol, 0.1 equiv) and Cs₂CO₃ (2.02 g, 6.190 mmol, 2 equiv). The resulting mixture was stirred for 1 h at 80° C. Desired product could be detected by LCMS. The reaction was quenched by the addition of water (5 mL). The resulting mixture was extracted with EtOAc. The combined organic layers were dried over anhydrous Na₂SO₄. After filtration, the filtrate was concentrated under reduced pressure. The residue was purified by silica gel column chromatography, eluted with petroleum ether/EtOAc (1:1) to afford 28b (440 mg, 35.42%) as a white solid. LCMS (ESI, m/z): [M+H]⁺ 402.2

### Step B. 1-[2-(2,6-dioxopiperidin-3-yl)-1-oxo-3H-isoindol-5-yl]piperidine-4-carbaldehyde (Int-28)

To a stirred mixture of 28b (440 mg, 1.096 mmol, 1 equiv) in THF (12.6 mL) was added 2 N HCl (0.76 mL, 1.520 mmol, 1.39 equiv). The resulting mixture was stirred for 2 h at room temperature. Desired product could be detected by LCMS. The reaction was quenched by the addition of saturated NaHCO₃ (aq.) (10 mL). The resulting mixture was extracted with EtOAc. The combined organic layers were dried over anhydrous Na₂SO₄. After filtration, the filtrate was concentrated under reduced pressure to afford Int-28 (300 mg, crude) as a white solid. LCMS (ESI, m/z): [M+H]⁺ 356.2

### 3-(1-oxo-5-(piperazin-1-yl)isoindolin-2-yl)piperidine-2,6-dione (Int-29)

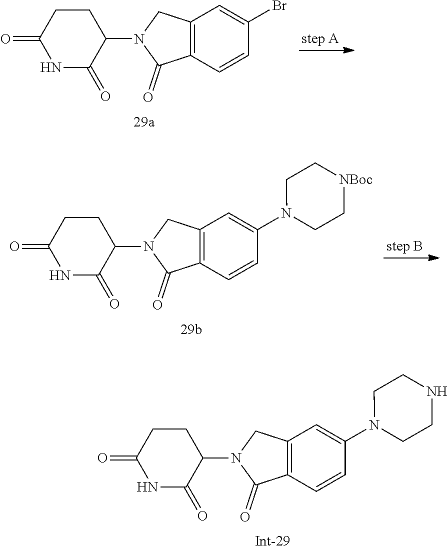 [SMILES: O=C1CCC(N2Cc3cc(Br)ccc3C2=O)C(=O)N1 ; CC(C)(C)OC(=O)N1CCN(c2ccc3c(c2)CN(C2CCC(=O)NC2=O)C3=O)CC1 ; O=C1CCC(N2Cc3cc(N4CCNCC4)ccc3C2=O)C(=O)N1]

### Step A. tert-butyl 4-(2-(2,6-dioxopiperidin-3-yl)-1-oxoisoindolin-5-yl)piperazine-1-carboxylate (29b)

To a stirred mixture of 29a (3 g, 9.284 mmol, 1 equiv) and tert-butyl piperazine-1-carboxylate (2.07 g, 11.141 mmol, 1.2 equiv) in DMF (60 mL) was added Cs₂CO₃ (9.07 g, 27.852 mmol, 3 equiv) and RuPhos Palladacycle Gen.3 (1.16 g, 1.393 mmol, 0.15 equiv) at room temperature under nitrogen atmosphere. The resulting mixture was stirred for overnight at 100° C. under nitrogen atmosphere. Desired product could be detected by LCMS. The resulting mixture was diluted with water (400 mL) and THF (100 mL). The resulting mixture was extracted with EtOAc (500 mL). The combined organic layers were washed with brine (200 mL), dried over anhydrous Na₂SO₄. After filtration, the filtrate was concentrated under reduced pressure. The residue was purified by silica gel column chromatography, eluted with CH₂Cl₂/ACN (1:9) to afford 29b (488 mg, 11.29%) as a light yellow solid. LCMS (ESI, m/z): [M+H]⁺ 429.2.

### Step B. 3-(1-oxo-5-(piperazin-1-yl)isoindolin-2-yl)piperidine-2,6-dione (Int-29)

To a stirred mixture of 29b (488 mg, 1.140 mmol, 1 equiv) in ACN (10 mL) was added 4M HCl (gas) in 1,4-dioxane (5 mL) at 0° C. The resulting mixture was stirred for 1 h at 25° C. The resulting mixture was concentrated under reduced pressure to afford Int-29 (500 mg, crude) as a white solid. The crude product was used in the next step directly without further purification. LCMS (ESI, m/z): [M+H]⁺ 329.3

### 2′-Chloro-7′-(2-methylcyclopentyl)spiro[cyclopropane-1,5′-pyrrolo[2,3-d]pyrimidin]-6′-one (Int-30)

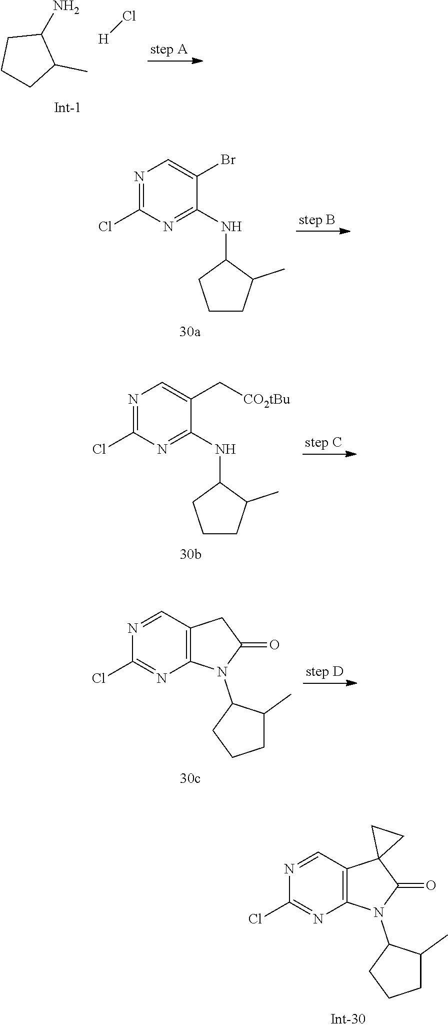

### Step A. 5-Bromo-2-chloro-N-(2-methylcyclopentyl)pyrimidin-4-amine (30a)

A solution of 5-bromo-2,4-dichloropyrimidine (7 g, 30.719 mmol, 1 eq.), Int-1 (3.66 g, 36.863 mmol, 1.2 eq.) and DIPEA (11.91 g, 92.157 mmol, 3 eq.) in THF was stirred for 4 h at room temperature under air atmosphere. Desired product could be detected by LCMS. The reaction was quenched by the addition of water (100 mL) at room temperature. The aqueous layer was extracted with EtOAc (3×100 mL). The combined organic layers were dried over anhydrous Na₂SO₄. After filtration, the filtrate was concentrated under reduced pressure. The residue was purified by silica gel column chromatography, eluted with petroleum ether/EtOAc (10:1) to afford 30a (6.5 g, 72.82%) as a white solid. LCMS (ESI, m/z): [M+H]⁺ 290.0.

### Step B. Tert-butyl 2-{2-chloro-4-[(2-methylcyclopentyl)amino]pyrimidin-5-yl}acetate (1f)

To a stirred solution of 30a (11.16 g, 38.396 mmol, 2 equiv) in dioxane (50 mL) were added Pd₂(dba)₃ (1.76 g, 1.920 mmol, 0.1 equiv) and tert-butyl 2-(bromozincio)acetate (5 g, 19.198 mmol, 1 equiv) under nitrogen atmosphere. The resulting mixture was stirred for 1 hour at 60° C. under nitrogen atmosphere. Desired product could be detected by LCMS. The resulting mixture was diluted with saturated aqueous NH₄C1 (50 mL). The resulting mixture was extracted with EtOAc (2×80 mL). The combined organic layers were washed with brine (15 mL), dried over anhydrous sodium sulfate. After filtration, the filtrate was concentrated under reduced pressure. The residue was purified by silica gel column chromatography, eluted with 0% to 15% EtOAc in petroleum ether. Pure fractions were evaporated to dryness to afford 30b (3 g, 43.16%) as a brown solid. LCMS (ESI, m/z): [M+H]⁺ 326.2.

### Step C. 2-Chloro-7-(2-methylcyclopentyl)-5H-pyrrolo[2,3-d]pyrimidin-6-one (30c)

A solution of 30b (6 g, 18.414 mmol, 1 equiv) in THF (60 mL) was treated with NaH (0.88 g, 36.828 mmol, 2 equiv) for 30 min at 25° C. under nitrogen atmosphere. The resulting mixture was stirred for 0.5 hours at 25° C. Desired product could be detected by LCMS. The resulting mixture was diluted with saturated aqueous NH₄C1 (20 mL). The resulting mixture was extracted with EtOAc (2×50 mL). The combined organic layers were washed with brine (15 mL), dried over anhydrous sodium sulfate. After filtration, the filtrate was concentrated under reduced pressure. The residue was purified by silica gel column chromatography, eluted with 0% to 15% EtOAc in petroleum ether. Pure fractions were evaporated to dryness to afford 30c (3.2 g, 63.52%) as a yellow solid. LCMS (ESI, m/z): [M+H]⁺ 252.1.

### Step D. 2′-Chloro-7′-(2-methylcyclopentyl)spiro[cyclopropane-1,5′-pyrrolo[2,3-d]pyrimidin]-6′-one (Int-30)

To a stirred solution of 30c (3.4 g, 13.508 mmol, 1 equiv) in DMSO (34 mL) were added diphenylvinylsulfonium triflate (6.36 g, 17.560 mmol, 1.3 equiv) and DBU (6.17 g, 40.524 mmol, 3 equiv). The resulting mixture was stirred for 1 hour at 25° C. under nitrogen atmosphere. Desired product could be detected by LCMS. The resulting mixture was diluted with saturated aqueous NH₄C1 (20 mL). The resulting mixture was extracted with EtOAc (2×30 mL). The combined organic layers were washed with brine (10 mL), dried over anhydrous sodium sulfate. After filtration, the filtrate was concentrated under reduced pressure. The residue was purified by silica gel column chromatography, eluted with 0% to 35% EtOAc in petroleum ether. Pure fractions were evaporated to dryness to afford Int-30 (3.2 g, 76.76%) as a green solid. LCMS (ESI, m/z): [M+H]⁺ 278.1.

### 2-(2,6-dioxopiperidin-3-yl)-1-oxo-3H-isoindole-5-carbaldehyde (Int-31)

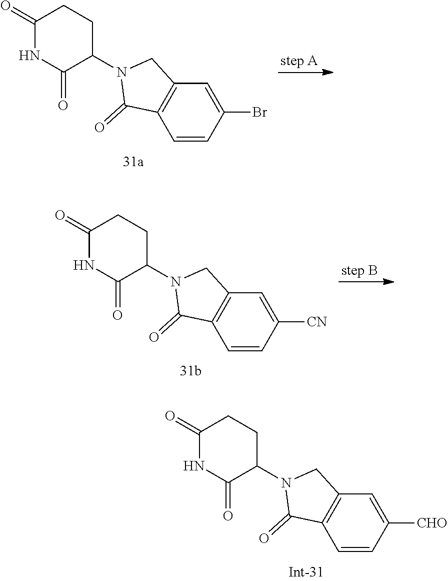 [SMILES: O=C1CCC(N2Cc3cc(Br)ccc3C2=O)C(=O)N1 ; N#Cc1ccc2c(c1)CN(C1CCC(=O)NC1=O)C2=O ; O=Cc1ccc2c(c1)CN(C1CCC(=O)NC1=O)C2=O]

### Step A. 2-(2,6-Dioxopiperidin-3-yl)-1-oxo-3H-isoindole-5-carbonitrile (31b)

To a mixture of 31a (3 g, 9.284 mmol, 1 equiv) in DMF (30 mL) was added DPPF (1.54 g, 2.785 mmol, 0.3 equiv), Zn(CN)₂ (1.64 g, 13.926 mmol, 1.5 equiv), Zn(OAc)₂ (0.51 g, 2.785 mmol, 0.3 equiv) and Pd₂(dba)₃ (0.85 g, 0.928 mmol, 0.1 equiv) at room temperature under argon atmosphere. The resulting mixture was stirred for overnight at 120° C. under argon atmosphere. The reaction was diluted with water. The resulting mixture was extracted with EtOAc (3×50 mL). The combined organic layers were washed with water (3×10 mL), dried over anhydrous Na₂SO₄. After filtration, the filtrate was concentrated under reduced pressure. The residue was purified by silica gel column chromatography, eluted with PE/EA (1:1) to afford 31b (1.8 g, 72.01%) as a white solid. LCMS (ESI, m/z): [M+H]⁺=269.

### Step B. 2-(2,6-Dioxopiperidin-3-yl)-1-oxo-3H-isoindole-5-carbaldehyde (Int-31)

To a mixture of 31b (600 mg, 2.228 mmol, 1 equiv) in Pyridine (6 mL)/H₂O (6 mL)/AcOH (3 mL) were added NaH₂PO₄ (561.43 mg, 4.679 mmol, 2.1 equiv) and Raney Ni (381.82 mg) at room temperature. The resulting mixture was stirred for overnight at 70° C. The reaction was monitored by TLC. The resulting mixture was concentrated under vacuum. The residue was purified by silica gel column chromatography, eluted with PE/EA (3:1) to afford Int-31 (215 mg, 35.44%) as a white solid. LCMS (ESI, m/z): [M+H]⁺=272.

### 3-(4-Bromo-3-methyl-2-oxo-1,3-benzodiazol-1-yl)-1-{[2-(trimethylsilyl)ethoxy]methyl}piperidine-2,6-dione (Int-32)

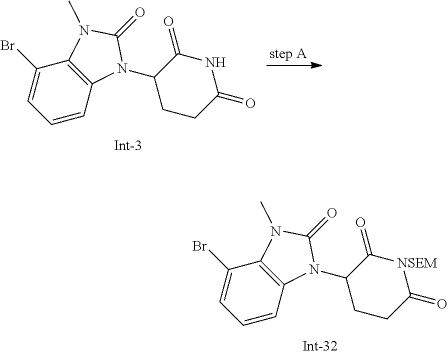 [SMILES: Cn1c(=O)n(C2CCC(=O)NC2=O)c2cccc(Br)c21 ; Cn1c(=O)n(C2CCC(=O)N(COCC[Si](C)(C)C)C2=O)c2cccc(Br)c21]

### Step A. 3-(4-Bromo-3-methyl-2-oxo-1,3-benzodiazol-1-yl)-1-{[2-(trimethylsilyl)ethoxy]methyl}piperidine-2,6-dione (Int-32)

To a mixture of Int-3 (1 g, 2.957 mmol, 1 equiv) and SEMCl (0.99 g, 5.914 mmol, 2 equiv) in DMF (10 mL) was added DIEA (1.53 g, 11.828 mmol, 4 equiv) at 0° C. The resulting mixture was stirred for 1 h at room temperature. The reaction was monitored by TLC (PE/EA=5/1, Rf=0.6). The resulting mixture was diluted with water and extracted with EtOAc (3×50 mL). The combined organic layers were washed with water (3×10 mL), dried over anhydrous Na₂SO₄. After filtration, the filtrate was concentrated under reduced pressure. The residue was purified by silica gel column chromatography, eluted with PE/EA (5:1) to afford Int-32 (1 g, 72.19%) as a white solid. LCMS (ESI, m/z): [M−H]⁺=466.

### 3-(5-Bromo-3-methyl-2-oxo-2,3-dihydro-1H-benzo[d]imidazol-1-yl)-1-((2-(trimethylsilyl)ethoxy)methyl)piperidine-2,6-dione (Int-33)

Compounds Int-33 was prepared as described for the synthesis of Int-32 using Int-4.

### 1-(1-Methyl-6-(piperazin-1-yl)-1H-indazol-3-yl)dihydropyrimidine-2,4(1H,3H)-dione (Int-34)

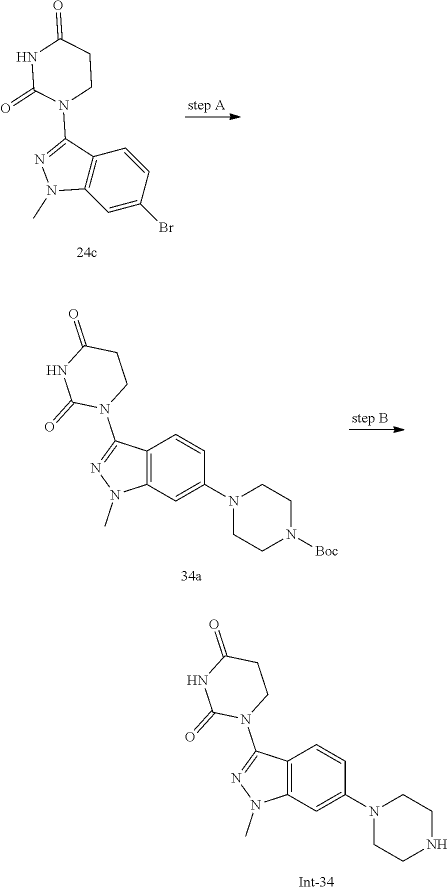 [SMILES: Cn1nc(N2CCC(=O)NC2=O)c2ccc(Br)cc21 ; Cn1nc(N2CCC(=O)NC2=O)c2ccc(N3CCN(C(=O)OC(C)(C)C)CC3)cc21 ; Cn1nc(N2CCC(=O)NC2=O)c2ccc(N3CCNCC3)cc21]

### Step A. Tert-butyl 4-[3-(2,4-dioxo-1,3-diazinan-1-yl)-1-methylindazol-6-yl]piperazine-1-carboxylate (34a)

To a solution of 24c (1 g, 3.095 mmol, 1 equiv) and tert-butyl piperazine-1-carboxylate (0.86 g, 4.643 mmol, 1.5 equiv) in DMF (15 mL) were added RuPhos Pd G3 (0.26 g, 0.310 mmol, 0.1 equiv) and Cs₂CO₃ (3.02 g, 9.285 mmol, 3 equiv) at room temperature under nitrogen atmosphere. The resulting mixture was stirred for 16 h at 100° C. under nitrogen atmosphere. Desired product could be detected by LCMS. The resulting mixture was diluted with water and extracted with EtOAc (3×30 mL). The combined organic layers were washed with brine, dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (10 mmol/L NH₄HCO₃), 10% to 35% gradient in 30 min; detector, UV 254 nm. The resulting mixture was concentrated under reduced pressure to afford 34a (430 mg, 32.43%) as a white solid. LCMS (ESI, m/z): [M+H]⁺=429.

### Step B. 1-(1-Methyl-6-(piperazin-1-yl)-1H-indazol-3-yl)dihydropyrimidine-2,4(1H,3H)-dione (Int-34)

To a stirred solution of 34a (430 mg, 1.004 mmol, 1 equiv) in MeCN (8 mL) was added 4M HCl (gas) in 1,4-dioxane (4 mL) dropwise at 0° C. The resulting mixture was stirred for 1 h at 0° C. Desired product could be detected by LCMS. The resulting mixture was concentrated under reduced pressure. The residue was purified by reversed-phase flash chromatography with the following conditions: column, C18 silica gel; mobile phase, MeCN in Water (10 mmol/L NH₄HCO₃), 10% to 20% gradient in 30 min; detector, UV 254 nm. The resulting mixture was concentrated under reduced pressure to afford Int-34 (250 mg, 75.87%) as a white solid. LCMS (ESI, m/z): [M+H]⁺=329.

### 2′-(((3R,4S)-1-((3-(1-Bromoethyl)phenyl)sulfonyl)-3-fluoropiperidin-4-yl)amino)-7′-((1R,3R)-3-((tetrahydro-2H-pyran-2-yl)oxy)cyclohexyl)spiro[cyclopropane-1,5′-pyrrolo[2,3-d]pyrimidin]-6′(7′H)-one (Int-35)

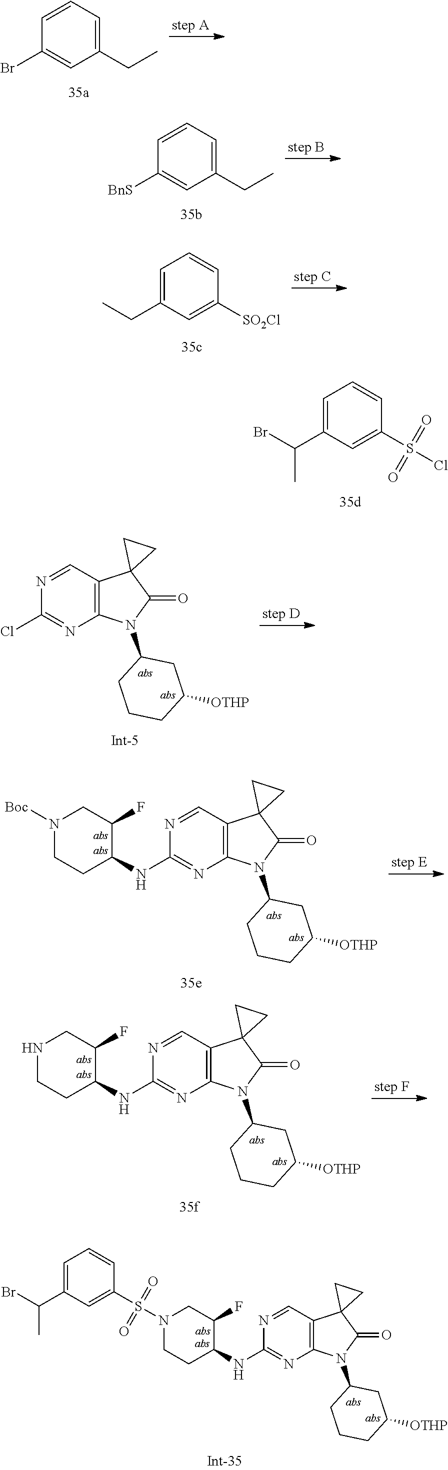

### Step A. 1-(Benzylsulfanyl)-3-ethylbenzene (35b)

To a solution of 35a (10 g, 54.035 mmol, 1 equiv) in dioxane (20 mL) were added phenylmethanethiol (8.05 g, 64.842 mmol, 1.2 equiv), Pd₂(dba)₃ (3.11 g, 5.404 mmol, 0.1 equiv), XantPhos (3.13 g, 5.404 mmol, 0.1 equiv) and DIEA (20.95 g, 162.105 mmol, 3 equiv) at 25° C. under nitrogen atmosphere. The resulting mixture was stirred for 1 hour at 80° C. under nitrogen atmosphere. The resulting mixture was diluted with saturated aqueous NH₄C1 (20 mL). The resulting mixture was extracted with EtOAc (2×30 mL). The combined organic layers were washed with brine, dried over anhydrous sodium sulfate. After filtration, the filtrate was concentrated under reduced pressure. The residue was purified by silica gel column chromatography, eluted with 0% to 15% EtOAc in PE. Pure fractions were evaporated to dryness to afford 35b (3 g, 19.45%) as a yellow oil.

### Step B. 3-Ethylbenzenesulfonyl chloride (35c)

To a stirred solution of 35b (3 g, 13.138 mmol, 1 equiv) in DCM (30 mL) were added H₂O (0.95 g, 52.552 mmol, 4 equiv), AcOH (3.16 g, 52.552 mmol, 4 equiv) and SO₂Cl₂ (7.09 g, 52.552 mmol, 4 equiv) at 0 degrees C. under nitrogen atmosphere. The resulting mixture was stirred for 0.5 hours at 0 degrees C. under nitrogen atmosphere. The resulting mixture was diluted with saturated aqueous NH₄C1 (20 mL). The resulting mixture was extracted with EtOAc (2×50 mL). The combined organic layers were washed with brine, dried over anhydrous sodium sulfate. After filtration, the filtrate was concentrated under reduced pressure. The residue was purified by silica gel column chromatography, eluted with PE. Pure fractions were evaporated to dryness to afford 35c (1.5 g, 50.21%) as a yellow oil.

### Step C. 3-(1-Bromoethyl)benzenesulfonyl chloride (35d)

To a stirred solution of 35c (2.6 g, 12.703 mmol, 1 equiv) in MeCN (30 mL) were added AIBN (208.60 mg, 1.270 mmol, 0.1 equiv) and NBS (2261.01 mg, 12.703 mmol, 1 equiv) under nitrogen atmosphere. The resulting mixture was stirred for 1 h at 80 degrees C. under nitrogen atmosphere. The resulting mixture was concentrated under reduced pressure. The residue was purified by silica gel column chromatography, eluted with 0% to 15% EtOAc in PE. Pure fractions were evaporated to dryness to afford 35d (1 g, 24.98%) as a yellow oil.

### Step D. Tert-butyl (3R,4S)-3-fluoro-4-((6′-oxo-7′-((1R,3R)-3-((tetrahydro-2H-pyran-2-yl) oxy) cyclohexyl)-6′,7′-dihydrospiro[cyclopropane-1,5′-pyrrolo[2,3-d]pyrimidin]-2′-yl)amino)piperidine-1-carboxylate (35e)

To a solution of Int-5 (160 mg, 0.423 mmol, 1 equiv) in dioxane (2 mL) were added tert-butyl (3R,4S)-4-amino-3-fluoropiperidine-1-carboxylate (92.42 mg, 0.423 mmol, 1 equiv), Pd₂(dba)₃ (38.46 mg, 0.042 mmol, 0.1 equiv), Cs₂CO₃ (413.88 mg, 1.269 mmol, 3 equiv) and RuPhos (19.76 mg, 0.042 mmol, 0.1 equiv) under nitrogen atmosphere. The resulting mixture was stirred for 12 hours at 90 degrees C. under nitrogen atmosphere. The residue was purified by reverse flash chromatography with the following conditions: column, C18 silica gel; mobile phase, MeCN in water, 5% to 50% gradient in 30 min; detector, UV 254 nm. Pure fractions were evaporated to dryness to afford 35e (100 mg, 40.09%) as a white solid. LCMS (ESI, m/z): [M+H]⁺=560.

### Step E. 2′-(((3R,4S)-3-Fluoropiperidin-4-yl) amino)-7′-((1R,3R)-3-((tetrahydro-2H-pyran-2-yl)oxy)cyclohexyl)spiro[cyclopropane-1,5′-pyrrolo[2,3-d]pyrimidin]-6′(7′H)-one (35f)

To a stirred solution of 35e (1 g, 1.787 mmol, 1 equiv) in DCM (5 mL) were added TEA (1.08 g, 10.722 mmol, 6 equiv) and TMSOTf (1.59 g, 7.148 mmol, 4 equiv). The resulting mixture was stirred for 1 hour at 0 degrees C. under nitrogen atmosphere. The resulting mixture was diluted with saturated aqueous NH₄C1 (20 mL). The resulting mixture was extracted with DCM (2×50 mL). The combined organic layers were washed with brine, dried over anhydrous sodium sulfate. After filtration, the filtrate was concentrated under reduced pressure to afford 1.1 g crude product and the residue was used for next step directly. LCMS (ESI, m/z): [M+H]⁺=460.

### Step F. 2′-(((3R,4S)-1-((3-(1-Bromoethyl)phenyl)sulfonyl)-3-fluoropiperidin-4-yl) amino)-7′-((1R,3R)-3-((tetrahydro-2H-pyran-2-yl)oxy)cyclohexyl)spiro[cyclopropane-1, 5′-pyrrolo[2,3-d]pyrimidin]-6′(7′H)-one (Int-35)

To a solution of 35f (410 mg, 0.580 mmol, 1 equiv) in DCM (10 mL) were added DIEA (674.97 mg, 5.223 mmol, 3 equiv) and 35d (493.61 mg, 1.741 mmol, 1 equiv). The resulting mixture was stirred for 1 hours at 25 degrees C. under nitrogen atmosphere. The resulting mixture was diluted with saturated aqueous NH₄C1 (20 mL). The resulting mixture was extracted with DCM (2×30 mL). The combined organic layers were washed with brine, dried over anhydrous sodium sulfate. After filtration, the filtrate was concentrated under reduced pressure. The residue was purified by silica gel column chromatography, eluted with 0% to 15% EtOAc in PE. Pure fractions were evaporated to dryness to afford Int-35 (900 mg, 65.85%) as a yellow solid. LCMS (ESI, m/z): [M+H]⁺=706.

### 2-(1-(2,6-Dioxopiperidin-3-yl)-3-methyl-2-oxo-2,3-dihydro-1H-benzo[d]imidazol-4-yl)acetic acid (Int-N1)

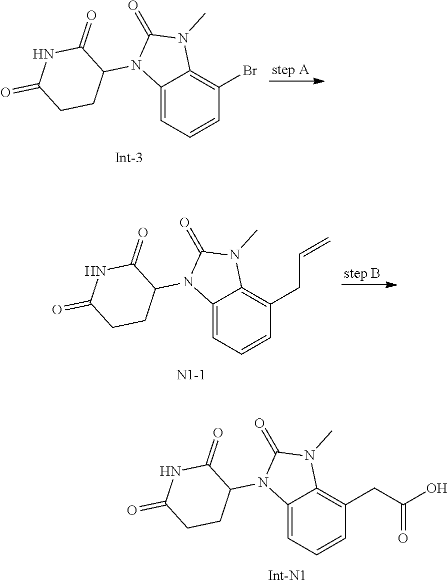 [SMILES: Cn1c(=O)n(C2CCC(=O)NC2=O)c2cccc(Br)c21 ; C=CCc1cccc2c1n(C)c(=O)n2C1CCC(=O)NC1=O ; Cn1c(=O)n(C2CCC(=O)NC2=O)c2cccc(CC(=O)O)c21]

### Step A. 3-(4-Allyl-3-methyl-2-oxo-2,3-dihydro-1H-benzo[d]imidazol-1-yl)piperidine-2,6-dione (N1-1)

To a stirred mixture of Int-3 (200 mg, 0.591 mmol, 1 equiv) and 4,4,5,5-tetramethyl-2-(prop-2-en-1-yl)-1,3,2-dioxaborolane (298.15 mg, 1.773 mmol, 3 equiv) in dioxane (3 mL) and H₂O (0.6 mL) were added Pd(DtBPF)Cl₂ (38.55 mg, 0.059 mmol, 0.1 equiv) and Cs₂CO₃ (578.10 mg, 1.773 mmol, 3 equiv) at room temperature under nitrogen atmosphere. The resulting mixture was stirred for 1 h at 80° C. under nitrogen atmosphere. Desired product could be detected by LCMS. The resulting mixture was diluted with water (20 mL). The resulting mixture was extracted with EtOAc (3×20 mL). The combined organic layers were dried over anhydrous Na₂SO₄. After filtration, the filtrate was concentrated under reduced pressure. The residue was purified by silica gel column chromatography, eluted with petroleum ether/EtOAc (1:1) to afford N1-1 (110 mg, 62.13%) as a brown solid. LCMS (ESI, m/z): [M+H]⁺ 300.1.

### Step B. 2-(1-(2,6-Dioxopiperidin-3-yl)-3-methyl-2-oxo-2,3-dihydro-1H-benzo[d]imidazol-4-yl)acetic acid (Int-N1)

To a stirred solution of N1-1 (110 mg, 0.367 mmol, 1 equiv) in ACN (2 mL) and CCl₄ (2 mL) were added NaIO₄ (165.25 mg, 1.101 mmol, 3 equiv) and RuCl₃·H₂O (8.28 mg, 0.037 mmol, 0.1 equiv) in H₂O (4 mL) dropwise at room temperature under nitrogen atmosphere. The resulting mixture was stirred for 1 h at room temperature under nitrogen atmosphere. Desired product could be detected by LCMS. The resulting mixture was concentrated under reduced pressure. The residue was purified by Prep-TLC (CH₂Cl₂/MeOH 5:1) to afford Int-N1 (90 mg, 77.18%) as a brown solid. LCMS (ESI, m/z): [M+H]⁺ 318.1.

### 3-[1-(2,6-Dioxopiperidin-3-yl)-3-methyl-2-oxo-1,3-benzodiazol-4-yl]propanoic acid (Int-N2)

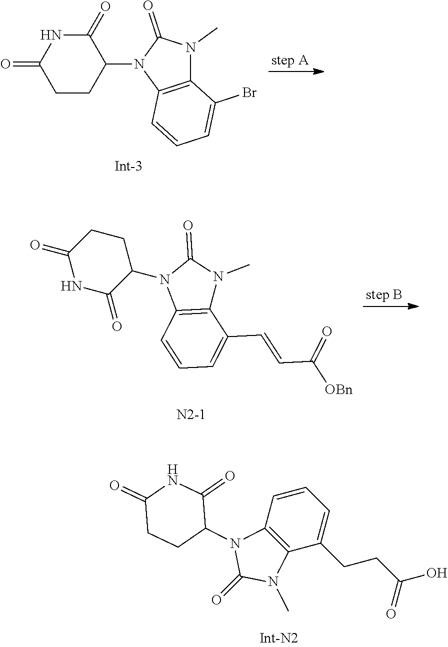 [SMILES: Cn1c(=O)n(C2CCC(=O)NC2=O)c2cccc(Br)c21 ; Cn1c(=O)n(C2CCC(=O)NC2=O)c2cccc(/C=C/C(=O)OCc3ccccc3)c21 ; "N2-1" ; Cn1c(=O)n(C2CCC(=O)NC2=O)c2cccc(CCC(=O)O)c21]

### Step A. Benzyl (2E)-3-[1-(2,6-dioxopiperidin-3-yl)-3-methyl-2-oxo-1,3-benzodiazol-4-yl]prop-2-enoate (N2-1)

To a stirred solution of Int-3 (500 mg, 1.479 mmol, 1 equiv) and Pd(AMPhos)₂Cl₂ (104.70 mg, 0.148 mmol, 0.1 equiv) in DMF (0.2 mL) was added Et₃N (4.49 g, 44.370 mmol, 30 equiv) and benzyl acrylate (479.76 mg, 2.958 mmol, 2 equiv) at room temperature under nitrogen atmosphere. The resulting mixture was stirred for 1 h at 80° C. under nitrogen atmosphere. The resulting mixture was diluted with water (20 mL). The resulting mixture was extracted with EtOAc (3×20 mL). The combined organic layers were dried over anhydrous Na₂SO₄. After filtration, the filtrate was concentrated under reduced pressure. The residue was purified by silica gel column chromatography, eluted with petroleum ether/EtOAc (1:1) to afford N2-1 (460 mg, 74.15%) as a yellow solid. LCMS (ESI, m/z): [M+H]⁺ 420.2

### Step B. 3-[1-(2,6-Dioxopiperidin-3-yl)-3-methyl-2-oxo-1,3-benzodiazol-4-yl]propanoic acid (Int-N2)

To a solution of N2-1 (1.70 g, 4.053 mmol, 1 equiv) in THF (30 mL) was added Pd/C (431.33 mg) and Pd(OH)₂/C (569.17 mg) in a pressure tank. The mixture was hydrogenated at room temperature under 30 psi of hydrogen pressure for 2 h, filtered through a Celite pad and concentrated under reduced pressure. The resulting mixture was concentrated under reduced pressure. The residue was purified by silica gel column chromatography, eluted with CH₂Cl₂/MeOH (30:1) to afford Int-N2 (1.1 g, 68.72%) as a white solid. LCMS (ESI, m/z): [M+H]⁺ 332.1

### 4-[1-(2,6-Dioxopiperidin-3-yl)-3-methyl-2-oxo-1,3-benzodiazol-4-yl]butanoic acid (Int-N3)

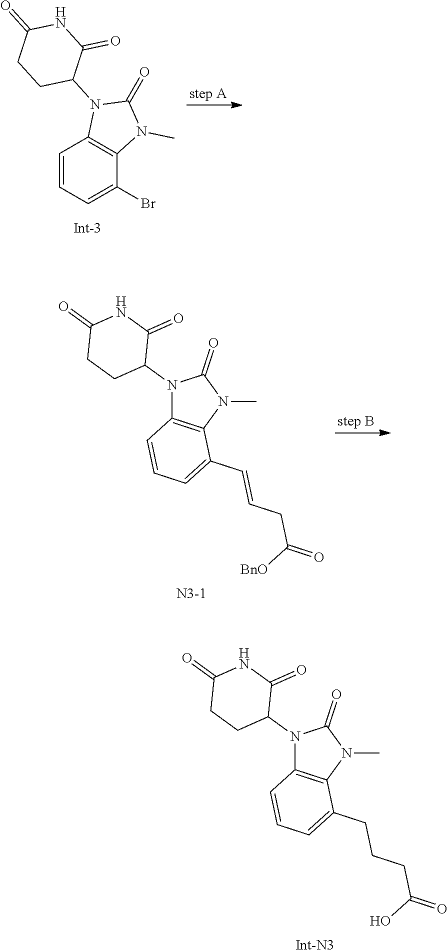 [SMILES: Cn1c(=O)n(C2CCC(=O)NC2=O)c2cccc(Br)c21 ; Cn1c(=O)n(C2CCC(=O)NC2=O)c2cccc(/C=C/CC(=O)OCc3ccccc3)c21 ; "N3-1" ; Cn1c(=O)n(C2CCC(=O)NC2=O)c2cccc(CCCC(=O)O)c21 ; "Int-N3"]

### Step A. Benzyl (3E)-4-[1-(2,6-dioxopiperidin-3-yl)-3-methyl-2-oxo-1,3-benzodiazol-4-yl]but-3-enoate (N3-1)

To a stirred mixture of Int-3 (500 mg, 1.479 mmol, 1 equiv) and benzyl but-3-enoate (521.10 mg, 2.958 mmol, 2 equiv) in DMF (6 mL, 6.461 mmol) was added Et₃N (4488.68 mg, 44.370 mmol, 30 equiv) and PdCl₂(AMPHOS)₂ (104.70 mg, 0.148 mmol, 0.1 equiv). The resulting mixture was stirred for 1 h at 80° C. under air hydrogen. Desired product could be detected by LCMS. The reaction was quenched by the addition of water (10 mL). The resulting mixture was extracted with EA. The combined organic layers were dried over anhydrous Na₂SO₄. After filtration, the filtrate was concentrated under reduced pressure. The residue was purified by silica gel column chromatography, eluted with petroleum ether/EtOAc (10:1) to afford N3-1 (534 mg, 74.24%) as a white solid. LCMS (ESI, m/z): [M+H]⁺ 434.2

### Step B. 4-[1-(2,6-Dioxopiperidin-3-yl)-3-methyl-2-oxo-1,3-benzodiazol-4-yl]butanoic acid (Int-N3)

To a stirred mixture of N3-1 (310 mg, 0.715 mmol, 1 equiv) in THF (5 mL) was added Pd/C (310 mg, 2.913 mmol, 4.07 equiv) and Pd(OH)₂/C (310 mg, 2.208 mmol, 3.09 equiv). The resulting mixture was stirred for 2 h at 25° C. under hydrogen. Desired product could be detected by LCMS. The resulting mixture was filtered, the filter cake was washed with MeOH. The filtrate was concentrated under reduced pressure to afford Int-N3 (267 mg, 88.64%) as a white solid. LCMS (ESI, m/z): [M+H]⁺ 346.1

### 5-[1-(2,6-Dioxopiperidin-3-yl)-3-methyl-2-oxo-1,3-benzodiazol-4-yl]pent-4-ynoic acid (Int-N4)

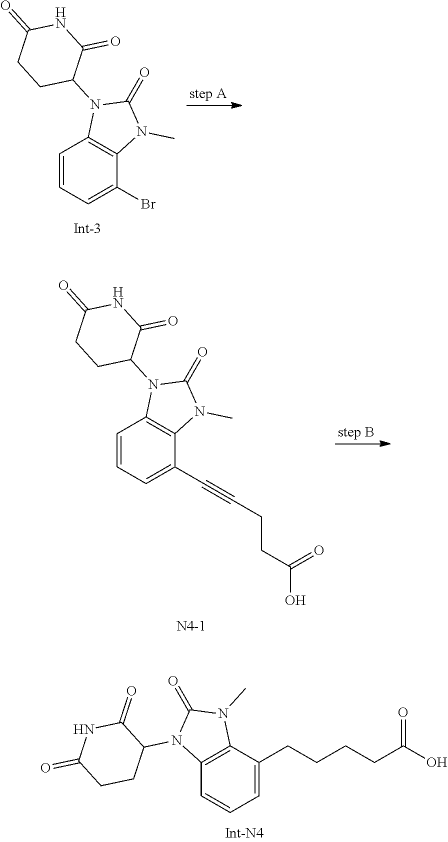 [SMILES: Cn1c(=O)n(C2CCC(=O)NC2=O)c2cccc(Br)c21 ; Cn1c(=O)n(C2CCC(=O)NC2=O)c2cccc(C#CCCC(=O)O)c21 ; "N4-1" ; Cn1c(=O)n(C2CCC(=O)NC2=O)c2cccc(CCCCC(=O)O)c21]

### Step B. 5-[1-(2,6-Dioxopiperidin-3-yl)-3-methyl-2-oxo-1,3-benzodiazol-4-yl]pent-4-ynoic acid (Int-N4)

To a mixture of Pd/C (1 g) and Pd(OH)₂/C (1 g) in tetrahydrofuran (50 mL) was added N4-1 (2.6 g, 7.317 mmol, 1 equiv) at 25 degrees C. under H₂ (1 atm). The resulting mixture was stirred for 2 h at 25 degrees C. Then the desired product was detected by LCMS. The resulting mixture was filtered, the filter cake was washed with MeOH (3×100 mL). The filtrate was concentrated under reduced pressure. The resulting mixture was used in the next step directly without further purification. This resulted in Int-N4 (2.7 g, 66.80%) as a yellow solid. LCMS (ESI, m/z): [M+H]⁺=360.

### 7-[1-(2,6-Dioxopiperidin-3-yl)-3-methyl-2-oxo-1,3-benzodiazol-4-yl]heptanoic acid (Int-N5)

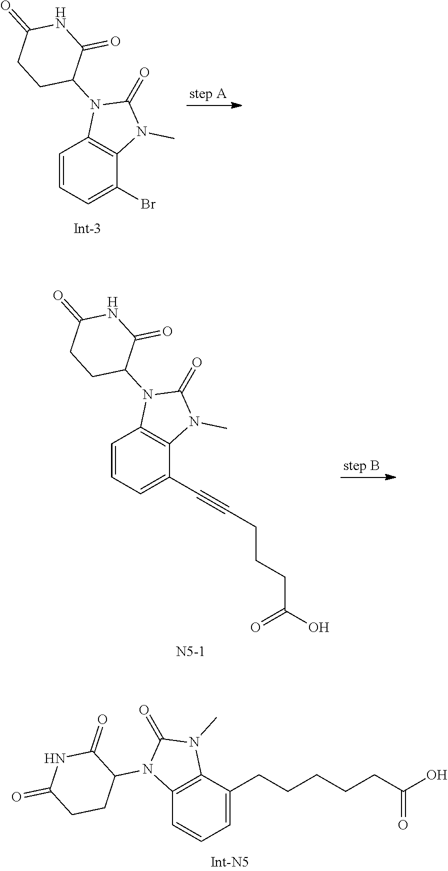 [SMILES: Cn1c(=O)n(C2CCC(=O)NC2=O)c2cccc(Br)c21 ; Cn1c(=O)n(C2CCC(=O)NC2=O)c2cccc(C#CCCCC(=O)O)c21 ; "N5-1" ; Cn1c(=O)n(C2CCC(=O)NC2=O)c2cccc(CCCCCC(=O)O)c21]

### Step A. 6-[1-(2,6-Dioxopiperidin-3-yl)-3-methyl-2-oxo-1,3-benzodiazol-4-yl]hex-5-ynoic acid (N5-1)

To a mixture of Int-3 (500 mg, 1.479 mmol, 1 equiv) and 5-hexynoic acid (331.58 mg, 2.958 mmol, 2 equiv), XPhos Pd G3 (250.31 mg, 0.296 mmol, 0.2 equiv) and XPhos (140.98 mg, 0.296 mmol, 0.2 equiv) and CuI (28.16 mg, 0.148 mmol, 0.1 equiv) in dimethylformamide (20 mL) was added TEA (10 mL) at 25 degrees C. under N2. The resulting mixture was stirred for 1 h at 80 degrees C. Then the desired product was detected by LCMS. The mixture was concentrated under vacuum to remove TEA. The residue was purified by reversed-phase Combi-Flash, eluted with CH₃CN:H₂O (0.5% MeOH)=5%-30%. This resulted in N4-1 (622 mg, 85.42%) as a yellow solid. LCMS (ESI, m/z): [M+H]⁺=370.

### Step B. 7-[1-(2,6-Dioxopiperidin-3-yl)-3-methyl-2-oxo-1,3-benzodiazol-4-yl]heptanoic acid (Int-N5)

To a mixture of Pd/C (300 mg) and Pd(OH)₂/C (300 mg) in tetrahydrofuran (40 mL) was added N5-1 (600 mg, 1.624 mmol, 1 equiv) at 25 degrees C. under H₂. The resulting mixture was stirred for 2 h at 25 degrees C. Then the desired product was detected by LCMS. The resulting mixture was filtered, the filter cake was washed with MeOH (3×100 mL). The filtrate was concentrated under reduced pressure. The resulting mixture was used in the next step directly without further purification. This resulted in Int-N5 (500 mg, 75.48%) as a yellow solid. LCMS (ESI, m/z): [M+H]⁺=374.

### 8-[1-(2,6-dioxopiperidin-3-yl)-3-methyl-2-oxo-1,3-benzodiazol-4-yl]octanoic acid (Int-N7)

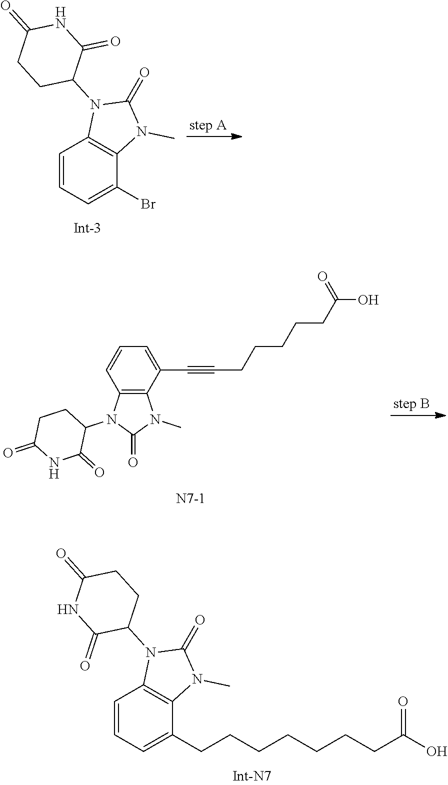

### Step A. 8-[1-(2,6-Dioxopiperidin-3-yl)-3-methyl-2-oxo-1,3-benzodiazol-4-yl]oct-7-ynoic acid (N7-1)

To a mixture of Int-3 (500 mg, 1.479 mmol, 1 equiv), 7-octynoic acid (414.54 mg, 2.958 mmol, 2 equiv), XPhos Pd G3 (250.31 mg, 0.296 mmol, 0.2 equiv) and XPhos (140.98 mg, 0.296 mmol, 0.2 equiv) and CuI (28.16 mg, 0.148 mmol, 0.1 equiv) in dimethylformamide (20 mL) was added TEA (10 mL) at 25 degrees C. under N₂. The resulting mixture was stirred for 1 h at 80 degrees C. Then the desired product was detected by LCMS. The mixture was concentrated under vacuum to remove TEA. The residue was purified by reversed-phase Combi-Flash, eluted with CH₃CN:H₂O (0.5% MeOH)=5%-30%. This resulted in N7-1 (433 mg, 67.79%) as a yellow solid. LCMS (ESI, m/z): [M+H]⁺=398.

### Step B. 8-[1-(2,6-Dioxopiperidin-3-yl)-3-methyl-2-oxo-1,3-benzodiazol-4-yl]octanoic acid (Int-N7)

To a mixture of Pd/C (200 mg) and Pd(OH)₂/C (200 mg) in tetrahydrofuran (30 mL) was added N7-1 (400 mg, 1.006 mmol, 1 equiv) at 25 degrees C. under H₂. The resulting mixture was stirred for 2 h at 25 degrees C. Then the desired product was detected by LCMS. The resulting mixture was filtered, the filter cake was washed with MeOH (3×100 mL). The filtrate was concentrated under reduced pressure. The resulting mixture was used in the next step directly without further purification. This resulted in Int-N7 (471 mg, 87.43%) as a yellow solid. LCMS (ESI, m/z): [M+H]⁺=402.

### 10-(1-(2,6-Dioxopiperidin-3-yl)-3-methyl-2-oxo-2,3-dihydro-1H-benzo[d]imidazol-4-yl)decanoic acid (Int-N9)

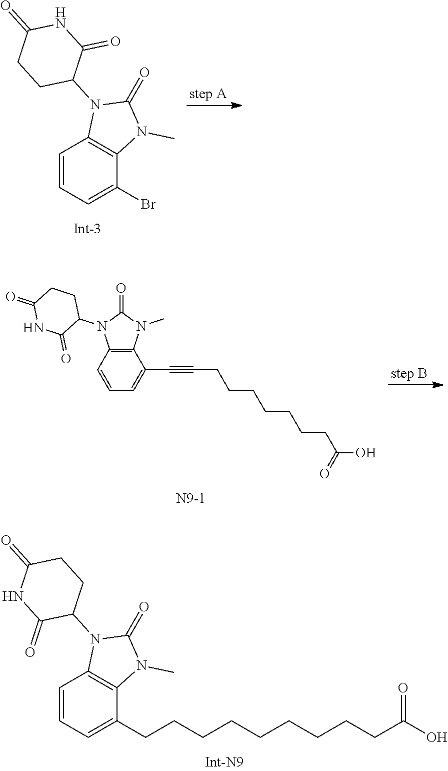 [SMILES: Cn1c(=O)n(C2CCC(=O)NC2=O)c2cccc(Br)c21 ; Cn1c(=O)n(C2CCC(=O)NC2=O)c2cccc(C#CCCCCCCCC(=O)O)c21 ; "N9-1" ; Cn1c(=O)n(C2CCC(=O)NC2=O)c2cccc(CCCCCCCCCC(=O)O)c21]

### Step A. 10-[1-(2,6-Dioxopiperidin-3-yl)-3-methyl-2-oxo-1,3-benzodiazol-4-yl]dec-9-ynoic acid (N9-1)

To a mixture of Int-3 (1 g, 2.957 mmol, 1 equiv) and 9-decynoic acid (1.00 g, 5.914 mmol, 2 equiv) and XPhos Pd G3 (0.50 g, 0.591 mmol, 0.2 equiv) and XPhos (0.28 g, 0.591 mmol, 0.2 equiv) and CuI (0.06 g, 0.296 mmol, 0.1 equiv) in dimethylformamide (40 mL) were added Et₃N (20 mL) at 25° C. under N₂. The resulting mixture was stirred for 1 h at 80° C. Then the desired product was detected by LCMS. The mixture was concentrated under vacuum to remove Et₃N. The combined organic layers were concentrated under reduced pressure. The residue was purified by reversed-phase Combi-Flash, eluted with CH₃CN:H₂O (0.5% MeOH)=5%-30%. This resulted in N9-1 (1.18 g, 84.40%) as a yellow solid. LCMS (ESI, m/z): [M+H]⁺ 426.2

### Step B. 10-(1-(2,6-Dioxopiperidin-3-yl)-3-methyl-2-oxo-2,3-dihydro-1H-benzo[d]imidazol-4-yl)decanoic acid (Int-N9)

To a stirred mixture of Pd/C (500 mg) and Pd(OH)₂/C (500 mg) in tetrahydrofuran (40 mL) were added N9-1 (1.1 g, 2.768 mmol, 1 equiv) at 25° C. under H₂. The resulting mixture was stirred for 2 h at 25° C. Then the desired product was detected by LCMS. The resulting mixture was filtered, the filter cake was washed with MeOH (3×100 mL). The filtrate was concentrated under reduced pressure. The resulting mixture was used in the next step directly without further purification. This resulted in Int-N9 (1 g, 77.40%) as a yellow solid. LCMS (ESI, m/z): [M+H]⁺ 430.2

### 2-(1-(2,6-Dioxopiperidin-3-yl)-3-methyl-2-oxo-2,3-dihydro-1H-benzo[d]imidazol-5-yl)acetic acid (Int-M1)

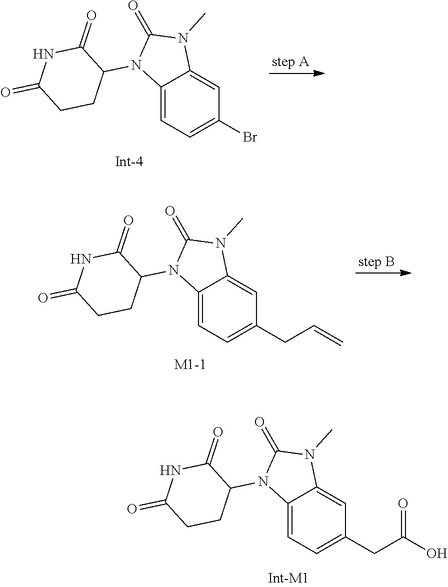 [SMILES: Cn1c(=O)n(C2CCC(=O)NC2=O)c2ccc(Br)cc21 ; C=CCc1ccc2c(c1)n(C)c(=O)n2C1CCC(=O)NC1=O ; Cn1c(=O)n(C2CCC(=O)NC2=O)c2ccc(CC(=O)O)cc21]

### Step A. 3-(5-Allyl-3-methyl-2-oxo-2,3-dihydro-1H-benzo[d]imidazol-1-yl)piperidine-2,6-dione (M1-1)

To a stirred mixture of Int-4 (200 mg, 0.591 mmol, 1 equiv) and 4,4,5,5-tetramethyl-2-(prop-2-en-1-yl)-1,3,2-dioxaborolane (298.15 mg, 1.773 mmol, 3 equiv) in dioxane (3 mL) and H₂O (0.6 mL) were added Pd(dtbpf)Cl₂ (38.55 mg, 0.059 mmol, 0.1 equiv) and Cs₂CO₃ (578.10 mg, 1.773 mmol, 3 equiv) at room temperature under nitrogen atmosphere. The resulting mixture was stirred for 1 h at 80° C. under nitrogen atmosphere. Desired product could be detected by LCMS. The resulting mixture was diluted with water (20 mL). The resulting mixture was extracted with EtOAc (3×20 mL). The combined organic layers were dried over anhydrous Na₂SO₄. After filtration, the filtrate was concentrated under reduced pressure. The residue was purified by silica gel column chromatography, eluted with petroleum ether/EtOAc (1:1) to afford M1-1 (110 mg, 62.13%) as a brown solid. LCMS (ESI, m/z): [M+H]⁺ 300.1.

### Step B. 2-(1-(2,6-Dioxopiperidin-3-yl)-3-methyl-2-oxo-2,3-dihydro-1H-benzo[d]imidazol-5-yl)acetic acid (Int-M1)

To a stirred solution of M1-1 (110 mg, 0.367 mmol, 1 equiv) in ACN (2 mL) and CCl₄ (2 mL) were added NaIO₄ (235.81 mg, 1.101 mmol, 3 equiv) and RuCl₃·H₂O (8.28 mg, 0.037 mmol, 0.1 equiv) in H₂O (4 mL) at room temperature under nitrogen atmosphere. The resulting mixture was stirred for 1 h at room temperature under nitrogen atmosphere. Desired product could be detected by LCMS. The resulting mixture was concentrated under reduced pressure. The residue was purified by Prep-TLC (CH₂Cl₂/MeOH 5:1) to afford Int-M1 (90 mg, 77.18%) as a brown solid. LCMS (ESI, m/z): [M+H]⁺ 318.1.

### 3-[1-(2,6-Dioxopiperidin-3-yl)-3-methyl-2-oxo-1,3-benzodiazol-5-yl]propanoic acid (Int-M2)

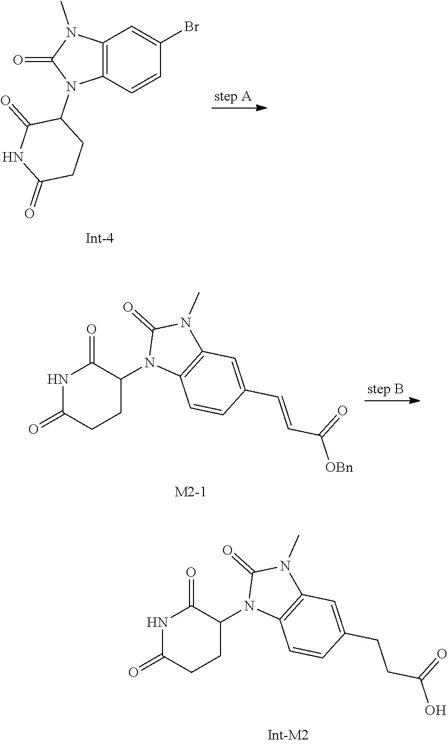 [SMILES: Cn1c(=O)n(C2CCC(=O)NC2=O)c2ccc(Br)cc21 ; Cn1c(=O)n(C2CCC(=O)NC2=O)c2ccc(/C=C/C(=O)OCc3ccccc3)cc21 ; Cn1c(=O)n(C2CCC(=O)NC2=O)c2ccc(CCC(=O)O)cc21]

### Step A. Benzyl (2E)-3-[1-(2,6-dioxopiperidin-3-yl)-3-methyl-2-oxo-1,3-benzodiazol-5-yl]prop-2-enoate (M2-1)

To a mixture of Int-4 (1.8 g, 5.322 mmol, 1 equiv) and benzyl prop-2-enoate (1.04 g, 6.38 mmol, 1.2 equiv) in Et₃N (10 mL)/DMF (20 mL) were added Pd(AMphos)₂Cl₂ (0.38 g, 0.532 mmol, 0.1 equiv) at room temperature under nitrogen atmosphere. The resulting mixture was stirred for additional 2 h at 80° C. under nitrogen atmosphere. Desired product could be detected by LCMS. The resulting mixture was diluted with water (50 mL). The resulting mixture was extracted with EtOAc (3×100 mL). The combined organic layers were dried over anhydrous sodium sulfate. After filtration, the filtrate was concentrated under reduced pressure. The residue was purified by silica gel column chromatography, eluted with 58% EtOAc in petroleum ether to afford M2-1 (1.7 g, 76.23%) as a yellow solid. LCMS (ESI, m/z): [M+H]⁺ 420.1.

### Step B. 3-[1-(2,6-Dioxopiperidin-3-yl)-3-methyl-2-oxo-1,3-benzodiazol-5-yl]propanoic acid (Int-M2)

To a solution of M2-1 (800 mg, 1.907 mmol, 1 equiv) in 15 mL THF was added Pd/C (10%, 800 mg) and Pd(OH)₂/C (800 mg, 5.697 mmol, 2.99 equiv) under nitrogen atmosphere in a 50 mL round-bottom flask. The mixture was hydrogenated at room temperature for 3 h under hydrogen atmosphere using a hydrogen balloon. The resulting mixture was filtered, the filter cake was washed with MeOH (3×200 mL). The filtrate was concentrated under reduced pressure. The crude product was used in the next step directly without further purification. This resulted in Int-M2 (1 g, 74.62%) as yellow solid. LCMS (ESI, m/z): [M+H]⁺ 332.1.

### 4-[1-(2,6-Dioxopiperidin-3-yl)-3-methyl-2-oxo-1,3-benzodiazol-5-yl]butanoic acid (Int-M3)

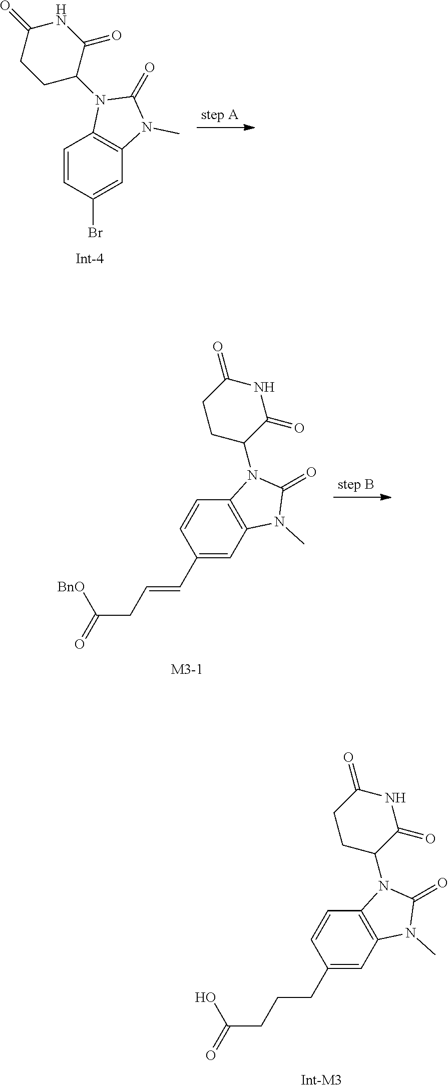 [SMILES: Cn1c(=O)n(C2CCC(=O)NC2=O)c2ccc(Br)cc21 ; Cn1c(=O)n(C2CCC(=O)NC2=O)c2ccc(/C=C/CC(=O)OCc3ccccc3)cc21 ; Cn1c(=O)n(C2CCC(=O)NC2=O)c2ccc(CCCC(=O)O)cc21]

### Step A. 2,6-Dichloro-3-iodopyridin-4-amine (benzyl (3E)-4-[1-(2,6-dioxopiperidin-3-yl)-3-methyl-2-oxo-1,3-benzodiazol-5-yl]but-3-enoate (M3-1)

To a mixture of Int-4 (1.2 g, 3.549 mmol, 1 equiv), benzyl but-3-enoate (1.88 g, 10.647 mmol, 3 equiv) and Pd(AMPhos)₂Cl₂ (0.25 g, 0.355 mmol, 0.1 equiv) in dimethylformamide (40 mL) was added TEA (20 mL) at 25 degrees C. under N2. The resulting mixture was stirred for 2 h at 80 degrees C. Then the desired product was detected by LCMS. The resulting mixture was diluted with water (100 mL) and extracted with EtOAc (3×100 mL). The combined organic layers were dried over anhydrous C and concentrated under reduced pressure. The residue was purified by silica gel column chromatography, eluted with DCM/EtOAc (1:1) to afford M3-1 (1 g, 57.86%) as a yellow solid. LCMS (ESI, m/z): [M+H]⁺=434.

### Step B. 4-[1-(2,6-Dioxopiperidin-3-yl)-3-methyl-2-oxo-1,3-benzodiazol-5-yl]butanoic acid (Int-M3)

To a mixture of Pd/C (500 mg) and Pd(OH)₂/C (500 mg) in tetrahydrofuran (50 mL) was added M3-1 (1 g, 2.307 mmol, 1 equiv) at 25 degrees C. under H₂. The resulting mixture was stirred for 2 h at 25 degrees C. Then the desired product was detected by LCMS. The resulting mixture was filtered, the filter cake was washed with MeOH (3×100 mL). The filtrate was concentrated under reduced pressure. The resulting mixture was used in the next step directly without further purification. This resulted in Int-M3 (800 mg, 86.35%) as a yellow solid. LCMS (ESI, m/z): [M+H]⁺=346.

### 5-[1-(2,6-Dioxopiperidin-3-yl)-3-methyl-2-oxo-1,3-benzodiazol-5-yl]pentanoic acid (Int-M4)

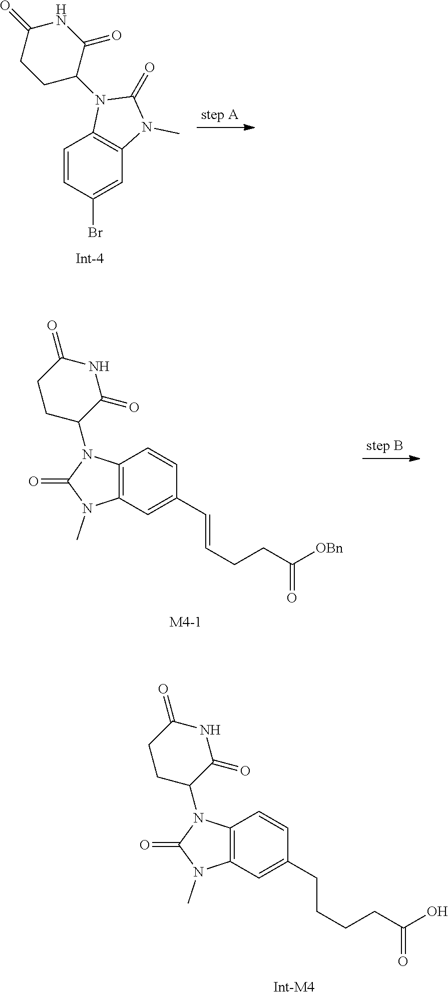 [SMILES: Cn1c(=O)n(C2CCC(=O)NC2=O)c2ccc(Br)cc21 ; Cn1c(=O)n(C2CCC(=O)NC2=O)c2ccc(/C=C/CCC(=O)OCc3ccccc3)cc21 ; Cn1c(=O)n(C2CCC(=O)NC2=O)c2ccc(CCCCC(=O)O)cc21]

### Step A. Benzyl 5-[1-(2,6-dioxopiperidin-3-yl)-3-methyl-2-oxo-1,3-benzodiazol-5-yl]pent-4-enoate (M4-1)

To a mixture of Int-4 (1.5 g, 4.436 mmol, 1 equiv) in Et₃N (15 mL) and DMF (30 mL) were added Pd(AMphos)₂Cl₂ (0.31 g, 0.444 mmol, 0.1 equiv) and benzyl pent-4-enoate (1.01 g, 5.323 mmol, 1.2 equiv) at 80° C. under nitrogen atmosphere. The resulting mixture was stirred for additional 2 h at 80° C. Desired product could be detected by LCMS. The mixture was allowed to cool down to room temperature. The resulting mixture was extracted with EtOAc (2×100 mL). The combined organic layers were washed with brine (3×100 mL), dried over anhydrous Na₂SO₄. The residue was purified by silica gel column chromatography, eluted with petroleum ether/EtOAc (3:2) to afford M4-1 (1.4 g, 70.53%) as a yellow solid. LCMS (ESI, m/z): [M+H]⁺ 448.2.

### Step B. 5-[1-(2,6-Dioxopiperidin-3-yl)-3-methyl-2-oxo-1,3-benzodiazol-5-yl]pentanoic acid (Int-M4)

To a solution of M4-1 (1.4 g, 3.129 mmol, 1 equiv) in THF (20 mL) was added Pd(OH)₂/C (10%, 1.4 g) under nitrogen atmosphere in a 100 mL round-bottom flask. The mixture was hydrogenated at room temperature for 4 h under hydrogen atmosphere using a hydrogen balloon. Desired product could be detected by LCMS. The resulting mixture was filtered through a Celite pad and concentrated under reduced pressure to afford Int-M4 (1 g, 88.94%) as a light yellow solid. The crude product was used in the next step directly without further purification. LCMS (ESI, m/z): [M+H]⁺ 360.2.

### 6-[1-(2,6-Dioxopiperidin-3-yl)-3-methyl-2-oxo-1,3-benzodiazol-5-yl]hexanoic acid (Int-M5)

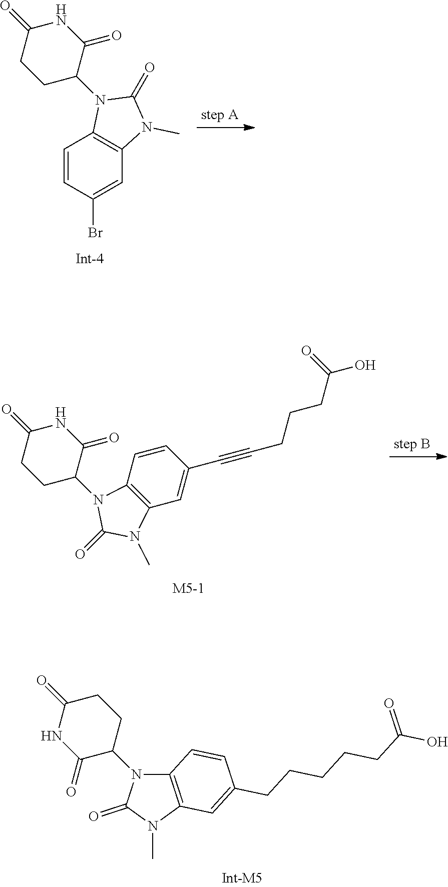 [SMILES: Cn1c(=O)n(C2CCC(=O)NC2=O)c2ccc(Br)cc21 ; Cn1c(=O)n(C2CCC(=O)NC2=O)c2ccc(C#CCCCC(=O)O)cc21 ; Cn1c(=O)n(C2CCC(=O)NC2=O)c2ccc(CCCCCC(=O)O)cc21]

### Step A. 5-[1-(2,6-Dioxopiperidin-3-yl)-3-methyl-2-oxo-1,3-benzodiazol-5-yl]pentanoic acid (M5-1)

To a mixture of Int-4 (0.7 g, 2.07 mmol, 1.00 equiv) and 5-hexynoic acid (0.3 g, 2.48 mmol, 1.2 equiv) in DMF was added Pd(dppf)Cl₂ (0.2 g, 0.21 mmol, 0.1 equiv) and Et₃N (6.3 g, 62.10 mmol, 30 equiv) at 80° C. under nitrogen atmosphere. The resulting mixture was stirred for additional 1 h at 80° C. The mixture was allowed to cool down to 25° C. The resulting mixture was concentrated under reduced pressure. The residue product was purified by reverse phase flash with the following conditions: MeCN in water (0.1% NH₄HCO₃), 5% to 13% gradient in 30 min; detector, UV 254 nm. Pure fractions were evaporated to dryness to afford M5-1 (500 mg, 65.39%) as a green solid. LCMS (ESI, m/z): [M+H]⁺ 370.1

### Step B. 6-[1-(2,6-Dioxopiperidin-3-yl)-3-methyl-2-oxo-1,3-benzodiazol-5-yl]hexanoic acid (Int-M5)

To a mixture of M5-1 (500 mg, 1.354 mmol, 1 equiv) in THF (40 mL) were added Pd/C (500 mg, 4.698 mmol, 3.47 equiv) and Pd(OH)₂/C (500 mg, 3.560 mmol, 2.63 equiv) at 20° C. The resulting mixture was stirred for 12 h at 25° C. under air hydrogen. Desired product could be detected by LCMS. The resulting mixture was filtered, the filter cake was washed with MeOH (3×200 mL). The filtrate was concentrated under reduced pressure. This resulted in Int-M5 (450 mg, 89.03%) as a yellow solid. The crude product was used in the next step directly without further purification. LCMS (ESI, m/z): [M+H]⁺ 374.2

### 8-[1-(2,6-dioxopiperidin-3-yl)-3-methyl-2-oxo-1,3-benzodiazol-5-yl]octanoic acid (Int-M7)

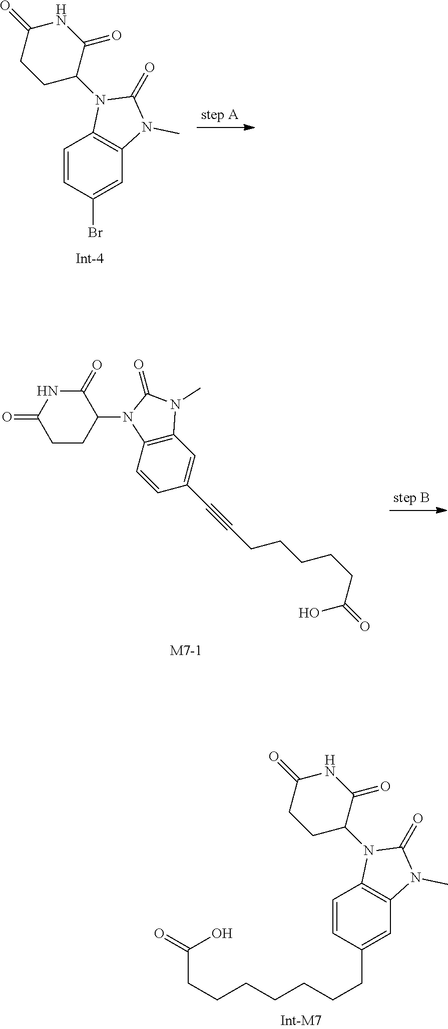 [SMILES: Cn1c(=O)n(C2CCC(=O)NC2=O)c2ccc(Br)cc21 ; Cn1c(=O)n(C2CCC(=O)NC2=O)c2ccc(C#CCCCCCC(=O)O)cc21 ; Cn1c(=O)n(C2CCC(=O)NC2=O)c2ccc(CCCCCCCC(=O)O)cc21]

### Step A. 8-[1-(2,6-Dioxopiperidin-3-yl)-3-methyl-2-oxo-1,3-benzodiazol-5-yl]oct-7-ynoic acid (M7-1)

To a stirred mixture of Int-4 (500 mg, 1.479 mmol, 1 equiv) and 7-octynoic acid (414.54 mg, 2.958 mmol, 2 equiv) in DMF (6 mL) and TEA (4488.68 mg, 44.370 mmol, 30 equiv) was added XPhos Pd G₃ (375.47 mg, 0.444 mmol, 0.3 equiv) and XPhos (211.47 mg, 0.444 mmol, 0.3 equiv) and CuI (14.08 mg, 0.074 mmol, 0.05 equiv). The resulting mixture was stirred for 1 h at 90° C. under nitrogen atmosphere. Desired product could be detected by LCMS. The reaction was quenched by the addition of water (10 mL). The resulting mixture was extracted with EtOAc. The combined organic layers were dried over anhydrous Na₂SO₄. After filtration, the filtrate was concentrated under reduced pressure. The residue was purified by reversed-phase Combi-Flash, eluted with CH₃CN:H₂O (0.5% NH₄HCO₃)=5%-60%. This resulted in M7-1 (524 mg, 73.03%) as a white solid. LCMS (ESI, m/z): [M+H]⁺ 398.2

### Step B. 8-[1-(2,6-Dioxopiperidin-3-yl)-3-methyl-2-oxo-1,3-benzodiazol-5-yl]octanoic acid (Int-M7)

To a stirred mixture of M7-1 (300 mg, 0.755 mmol, 1 equiv) in THF (5 mL) was added Pd/C (160 mg, 1.503 mmol, 1.99 equiv) and Pd(OH)₂/C (160 mg, 1.139 mmol, 1.51 equiv). The resulting mixture was stirred for 12 h at 25° C. under air hydrogen. Desired product could be detected by LCMS. The resulting mixture was filtered, the filter cake was washed with MeOH. The filtrate was concentrated under reduced pressure to afford Int-M7 (278 mg, 44.03%) as a white solid. LCMS (ESI, m/z): [M+H]⁺ 402.2

### 10-[1-(2,6-Dioxopiperidin-3-yl)-3-methyl-2-oxo-1,3-benzodiazol-5-yl]decanoic acid (Int-M9)

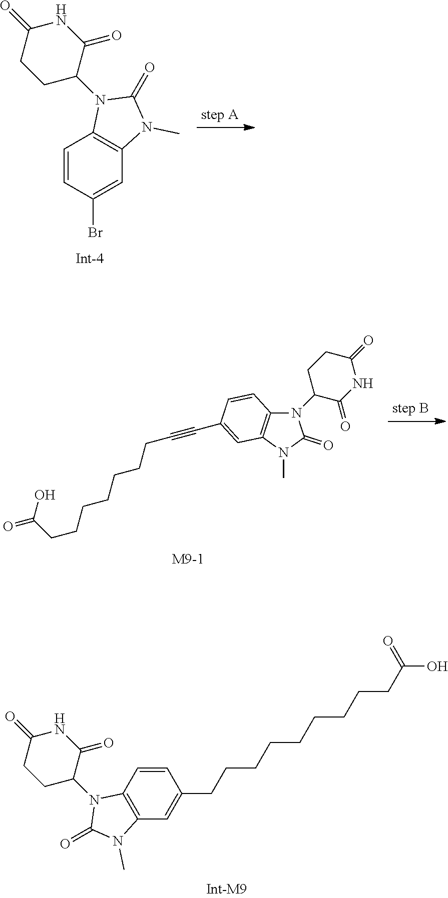 [SMILES: Cn1c(=O)n(C2CCC(=O)NC2=O)c2ccc(Br)cc21 ; Cn1c(=O)n(C2CCC(=O)NC2=O)c2ccc(C#CCCCCCCCC(=O)O)cc21 ; Cn1c(=O)n(C2CCC(=O)NC2=O)c2ccc(CCCCCCCCCC(=O)O)cc21]

### Step B. 10-[1-(2,6-Dioxopiperidin-3-yl)-3-methyl-2-oxo-1,3-benzodiazol-5-yl]decanoic acid (Int-M9)

To a solution of M9-1 (540 mg, 1.269 mmol, 1 equiv) in 20 mL THF was added Pd/C (10%, 250 mg) under nitrogen atmosphere in a 50 mL round-bottom flask. The mixture was hydrogenated at room temperature for 3 h under hydrogen atmosphere using a hydrogen balloon, filtered through a Celite pad and concentrated under reduced pressure. Desired product could be detected by LCMS. The resulting mixture was filtered, the filter cake was washed with THF (3×10 mL). The filtrate was concentrated under reduced pressure. This resulted in Int-M9 (300 mg, 55.03%) as a yellow solid. LCMS (ESI, m/z): [M+H]⁺ 430.2.

### EXEMPLARY COMPOUNDS

### Example 1. 3-{3-Methyl-4-[6-(3-{4-[7′-(2-methylcyclopentyl)-6′-oxospiro[cyclopropane-1,5′-pyrrolo[2,3-d]pyrimidin]-2′-ylamino]piperidin-1-ylsulfonyl}pyrrolidin-1-yl)-6-oxohexyl]-2-oxo-1,3-benzodiazol-1-yl}piperidine-2,6-dione (1)

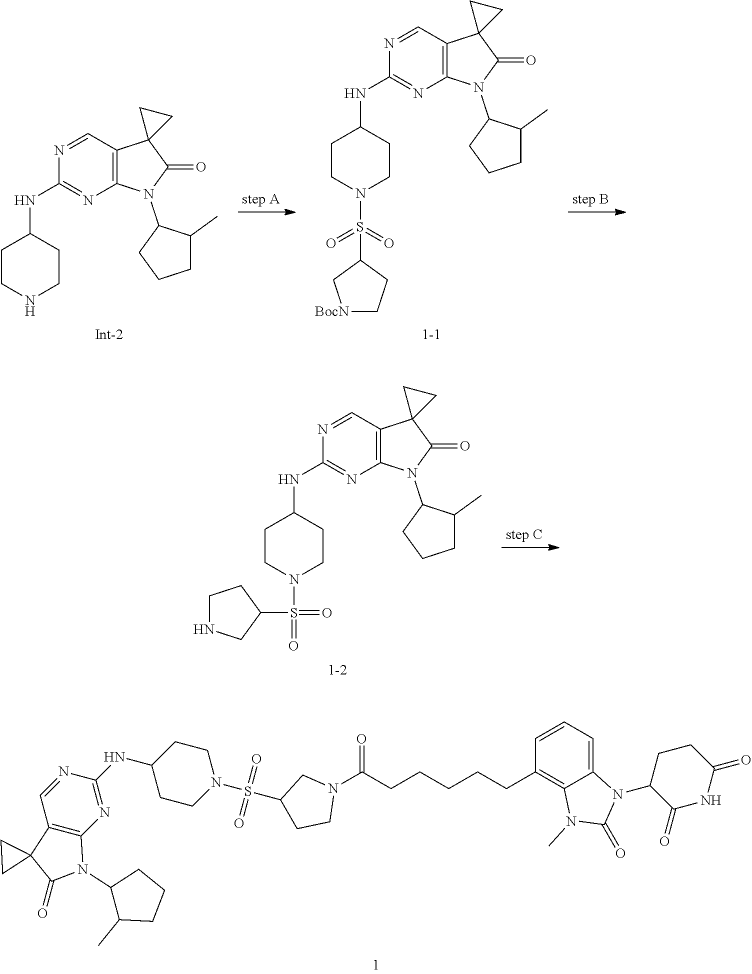 [SMILES: CC1CCCC1N1C(=O)C2(CC2)c2cnc(NC3CCNCC3)nc21 ; CC1CCCC1N1C(=O)C2(CC2)c2cnc(NC3CCN(S(=O)(=O)C4CCN(C(=O)OC(C)(C)C)C4)CC3)nc21 ; CC1CCCC1N1C(=O)C2(CC2)c2cnc(NC3CCN(S(=O)(=O)C4CCNC4)CC3)nc21 ; CC1CCCC1N1C(=O)C2(CC2)c2cnc(NC3CCN(S(=O)(=O)C4CCN(C(=O)CCCCCc5cccc6c5n(C)c(=O)n6C5CCC(=O)NC5=O)C4)CC3)nc21]

### Step A. Tert-butyl 3-{4-[7′-(2-methylcyclopentyl)-6′-oxospiro[cyclopropane-1,5′-pyrrolo[2,3-d]pyrimidin]-2′-ylamino]piperidin-1-ylsulfonyl}pyrrolidine-1-carboxylate (1-1)

To a mixture of Int-2 (200 mg, 0.586 mmol, 1 equiv) in THF (10 mL) were added TEA (2.44 mL, 17.580 mmol, 30 equiv) and tert-butyl 3-(chlorosulfonyl)pyrrolidine-1-carboxylate (189.59 mg, 0.703 mmol, 1.2 equiv) at room temperature. The resulting mixture was stirred for 2 h at room temperature. The resulting mixture was extracted with EtOAc (3×30 mL), dried over anhydrous C. After filtration, the filtrate was concentrated under reduced pressure. The residue was purified by Pre-TLC (CH2Cl2/MeOH (10:1)) to afford 1-1 (139 mg, 41.29%) as a yellow solid. LCMS (ESI, m/z): [M+H]⁺=575

### Step B. 7′-(2-Methylcyclopentyl)-2′-((1-(pyrrolidin-3-ylsulfonyl)piperidin-4-yl)amino)spiro [cyclopropane-1,5′-pyrrolo[2,3-d]pyrimidin]-6′(7′H)-one (1-2)

To a stirred mixture of 1-1 (40 mg, 0.070 mmol, 1 equiv) in ACN (2 mL) was added 4M HCl in dioxane (0.4 mL) dropwise at 0° C. The resulting mixture was stirred for 30 min at 0° C. The resulting mixture was concentrated under reduced pressure to afford 1-2 (40 mg, crude) as a white solid. The crude product mixture was used in the next step directly without further purification. LCMS (ESI, m/z): [M+H]⁺=475.25

### Step C. 3-{3-Methyl-4-[6-(3-{4-[7′-(2-methylcyclopentyl)-6′-oxospiro[cyclopropane-1,5′-pyrrolo[2,3-d]pyrimidin]-2′-ylamino]piperidin-1-ylsulfonyl}pyrrolidin-1-yl)-6-oxohexyl]-2-oxo-1,3-benzodiazol-1-yl}piperidine-2,6-dione (1)

To a stirred mixture of 1-2 (40 mg, crude) in DMF (2 mL) were added DIEA (217.85 mg, 1.680 mmol, 20 equiv), EDCI (24.23 mg, 0.126 mmol, 1.5 equiv), 1lambda4-pyridine-1,2-diol (14.17 mg, 0.126 mmol, 1.5 equiv), Int-N7 (34.62 mg, 0.092 mmol, 1.1 equiv) dropwise at room temperature. The resulting mixture was stirred for overnight at room temperature. The resulting mixture was purified by Prep-HPLC with the following conditions (Column: YMC-Actus Triart C18 ExRS Column, 30*150 mm, 5 μm; Mobile Phase A: Water (10 mmol/L NH4HCO3+0.05% NH3H2O), Mobile Phase B: MEOH; Flow rate: 60 mL/min mL/min; Gradient: 62% Bto77% Bin10 min; Wave Length: 254 nm/220 nm nm; RT1 (min): 9.9) to afford compound 1 (11 mg, 15.21%) as a white solid.

### Example 2. 3-{3-Methyl-4-[8-(7-{4-[7′-(2-methylcyclopentyl)-6′-oxospiro[cyclopropane-1,5′-pyrrolo[2,3-d]pyrimidin]-2′-ylamino]piperidin-1-ylsulfonyl}-1,7-diazaspiro[4.4]nonan-1-yl)-8-oxooctyl]-2-oxo-1,3-benzodiazol-1-yl}piperidine-2,6-dione (2)

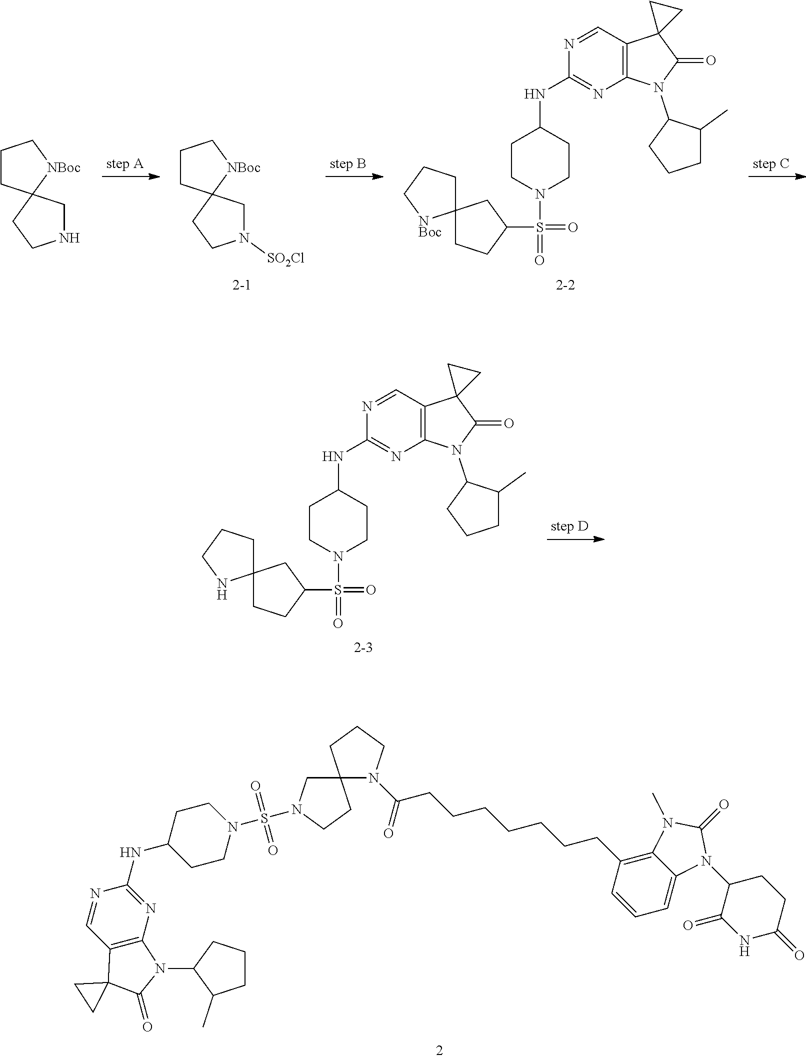 [SMILES: CC(C)(C)OC(=O)N1CCCC12CCNC2 ; CC(C)(C)OC(=O)N1CCCC12CCN(S(=O)(=O)Cl)C2 ; CC1CCCC1N1C(=O)C2(CC2)c2cnc(NC3CCN(S(=O)(=O)C4CCC5(CCCN5C(=O)OC(C)(C)C)C4)CC3)nc21 ; CC1CCCC1N1C(=O)C2(CC2)c2cnc(NC3CCN(S(=O)(=O)C4CCC5(CCCN5)C4)CC3)nc21 ; CC1CCCC1N1C(=O)C2(CC2)c2cnc(NC3CCN(S(=O)(=O)N4CCC5(CCCN5C(=O)CCCCCCCc5cccc6c5n(C)c(=O)n6C5CCC(=O)NC5=O)C4)CC3)nc21]

### Step A. Tert-butyl 7-(chlorosulfonyl)-1,7-diazaspiro[4.4]nonane-1-carboxylate (2-1)

To a stirred mixture of tert-butyl 1,7-diazaspiro[4.4]nonane-1-carboxylate (700 mg, 3.093 mmol, 1 equiv) and Pyridine (489.31 mg, 6.186 mmol, 2 equiv) in DCM (7 mL) was added SO2Cl2 (626.14 mg, 4.639 mmol, 1.5 equiv) in portions at 0° C. The resulting mixture was stirred for 16 h at 25° C. The reaction was quenched by the addition of 1N HCl (18 mL). The resulting mixture was extracted with CH₂Cl₂. The combined organic layers were washed with brine, dried over anhydrous C. After filtration, the filtrate was concentrated under reduced pressure to afford 2-1 (800 mg, crude) as a white solid. The crude product mixture was used in the next step directly without further purification. LCMS (ESI, m/z): [M+H−56]⁺=269.

Compound 2 was prepared following the procedure for the synthesis of compound 1 (step A,B,C) in example 1.

### Example 13: N-(3-{4-[1-(2,6-Dioxopiperidin-3-yl)-3-methyl-2-oxo-1,3-benzodiazol-4-yl]piperidin-1-yl}propyl)-N-methyl-4-{7′-[(1R,2S)-2-methylcyclopentyl]-6′-oxospiro[cyclopropane-1,5′-pyrrolo[2,3-d]pyrimidin]-2′-ylamino}piperidine-1-sulfonamide (13)

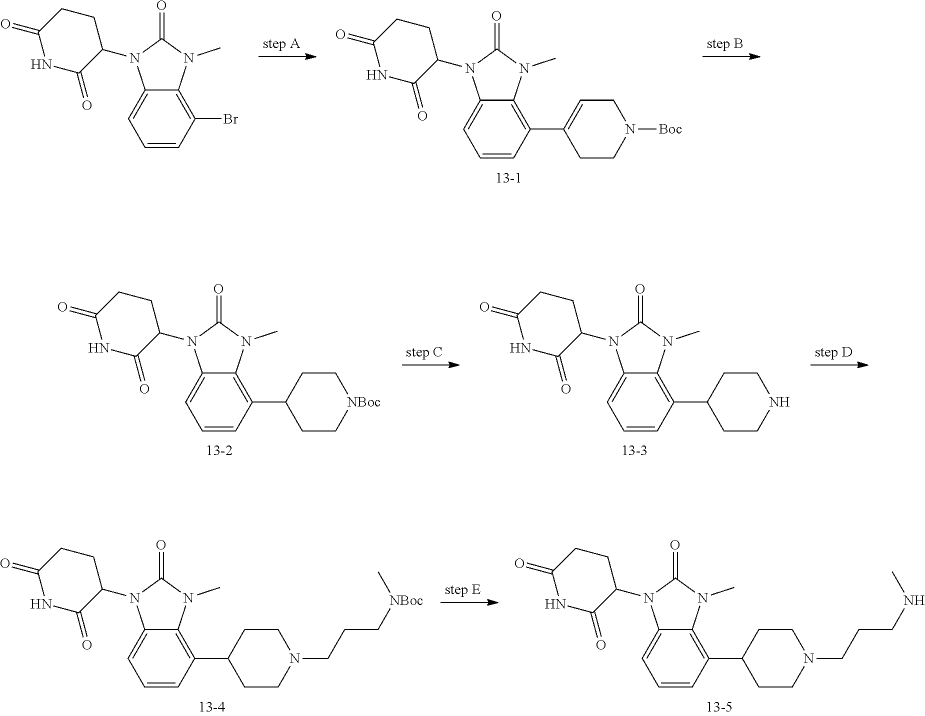 [SMILES: Cn1c(=O)n(C2CCC(=O)NC2=O)c2cccc(Br)c21 ; Cn1c(=O)n(C2CCC(=O)NC2=O)c2cccc(C3=CCN(C(=O)OC(C)(C)C)CC3)c21 ; Cn1c(=O)n(C2CCC(=O)NC2=O)c2cccc(C3CCN(C(=O)OC(C)(C)C)CC3)c21 ; Cn1c(=O)n(C2CCC(=O)NC2=O)c2cccc(C3CCNCC3)c21 ; CN(CCCN1CCC(c2cccc3c2n(C)c(=O)n3C2CCC(=O)NC2=O)CC1)C(=O)OC(C)(C)C ; CNCCCN1CCC(c2cccc3c2n(C)c(=O)n3C2CCC(=O)NC2=O)CC1]

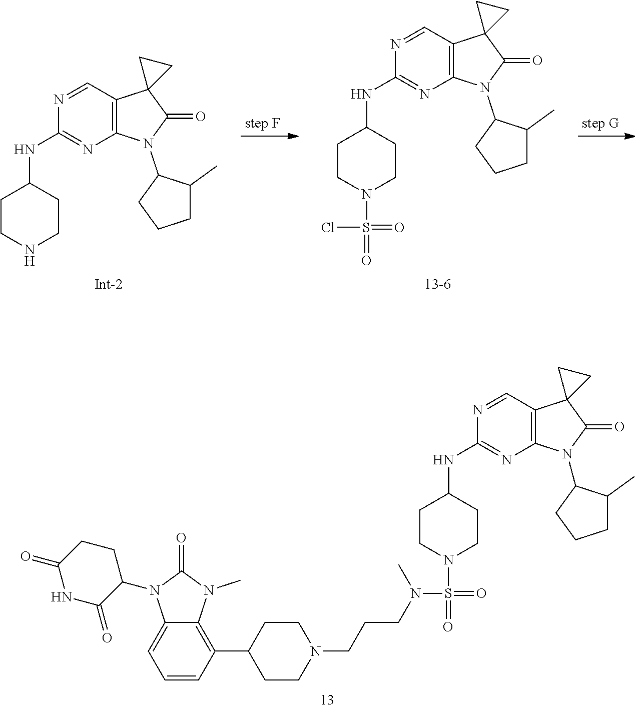 [SMILES: CC1CCCC1N1C(=O)C2(CC2)c2cnc(NC3CCNCC3)nc21 ; CC1CCCC1N1C(=O)C2(CC2)c2cnc(NC3CCN(S(=O)(=O)Cl)CC3)nc21 ; CC1CCCC1N1C(=O)C2(CC2)c2cnc(NC3CCN(S(=O)(=O)N(C)CCCN4CCC(c5cccc6c5n(C)c(=O)n6C5CCC(=O)NC5=O)CC4)CC3)nc21]

### Step A. Tert-butyl 4-[1-(2,6-dioxopiperidin-3-yl)-3-methyl-2-oxo-1,3-benzodiazol-4-yl]-3,6-dihydro-2H-pyridine-1-carboxylate (13-1)

To a stirred mixture of 3-(4-bromo-3-methyl-2-oxo-1,3-benzodiazol-1-yl)piperidine-2,6-dione (1 g, 2.957 mmol, 1 equiv) and tert-butyl 4-(4,4,5,5-tetramethyl-1,3,2-dioxaborolan-2-yl)-3,6-dihydro-2H-pyridine-1-carboxylate (1.19 g, 3.844 mmol, 1.3 equiv) in dioxane (10 mL) and H₂O (1 mL) was added Xphos Pd G3 (0.38 g, 0.444 mmol, 0.15 equiv) and K₃PO₄ (1.88 g, 8.871 mmol, 3 equiv). The resulting mixture was stirred for 3 h at 60° C. The resulting mixture was concentrated under reduced pressure. The residue was purified by silica gel column chromatography, eluted with DCM/MeOH (20:1) to afford 13-1 (1.1 g, 80.22%) as a white solid. LCMS (ESI, m/z): [M+H]⁺=441

### Step B. Tert-butyl 4-[1-(2,6-dioxopiperidin-3-yl)-3-methyl-2-oxo-1,3-benzodiazol-4-yl]piperidine-1-carboxylate (13-2)

To a stirred mixture of 13-1 (1.1 g, 2.497 mmol, 1 equiv) in THF (13 mL) were added Pd/C (0.55 g, 5.169 mmol, 2.07 equiv) and Pd(OH)₂/C (0.55 g, 3.920 mmol, 1.57 equiv). The resulting mixture was stirred for 3 h at 50° C. under hydrogen atmosphere. The resulting mixture was filtered, the filter cake was washed with THF. The filtrate was concentrated under reduced pressure to afford 13-2 (1 g, 84.16%) as a white solid. LCMS (ESI, m/z): [M+H]⁺=443.

### Step C. 3-[3-Methyl-2-oxo-4-(piperidin-4-yl)-1,3-benzodiazol-1-yl]piperidine-2,6-dione (13-3)

To a stirred mixture of 13-2 (1 g, 2.260 mmol, 1 equiv) in DCM (3 mL) was added 4M HCl (gas) in 1,4-dioxane (6 mL). The resulting mixture was stirred for 1 h at 25° C. The resulting mixture was concentrated under reduced pressure to afford 3-[3-methyl-2-oxo-4-(piperidin-4-yl)-1,3-benzodiazol-1-yl]piperidine-2,6-dione 13-3 (850 mg, crude) as a white solid. The crude product was used in the next step directly without further purification. LCMS (ESI, m/z): [M+H]⁺=343.

### Step D. Tert-butyl N-(3-{4-[1-(2,6-dioxopiperidin-3-yl)-3-methyl-2-oxo-1,3-benzodiazol-4-yl]piperidin-1-yl}propyl)-N-methylcarbamate (13-4)

To a stirred mixture of 13-3 (500 mg, 1.460 mmol, 1 equiv) and tert-butyl N-methyl-N-(3-oxopropyl)carbamate (546.84 mg, 2.920 mmol, 2 equiv) in THF (5 mL) and DMF (1 mL) was added AcOH (0.2 mL). The resulting mixture was stirred for 0.5 h at 25° C. Then NaBH(OAc)₃ (810.87 mg, 3.825 mmol, 2.62 equiv) was added to the mixture. The resulting mixture was stirred for 1 h at 25° C. The resulting mixture was concentrated under reduced pressure. The reaction was quenched by the addition of water (10 mL). The resulting mixture was extracted with EtOAc. The combined organic layers were dried over anhydrous C. After filtration, the filtrate was concentrated under reduced pressure. The residue was purified by silica gel column chromatography, eluted with MeOH/DCM (1:30) to afford 13-4 (400 mg, 52.26%) as a white solid. LCMS (ESI, m/z): [M+H]⁺=514.

### Step E. 3-(3-Methyl-4-(1-(3-(methylamino)propyl)piperidin-4-yl)-2-oxo-2,3-dihydro-1H-benzo[d]imidazol-1-yl)piperidine-2,6-dione (13-5)

To a stirred mixture of 13-4 (71 mg, 0.138 mmol, 1 equiv) in ACN (5 mL) was added 4M HCl (gas) in 1,4-dioxane (0.5 mL). The resulting mixture was stirred for 1 h at 25° C. The resulting mixture was concentrated under reduced pressure to afford 13-5 (65 mg, crude) as a white solid. The crude product was used in the next step directly without further purification. LCMS (ESI, m/z): [M+H]⁺=414.

### Step F. 4-[7′-(2-Methylcyclopentyl)-6′-oxospiro[cyclopropane-1,5′-pyrrolo[2,3-d]pyrimidin]-2′-ylamino]piperidine-1-sulfonyl chloride (13-6)

To a stirred mixture of Int-2 (380 mg, 1.113 mmol, 1 equiv) and DIPEA (575.34 mg, 4.452 mmol, 4 equiv) in DCM (4 mL) was added SO₂Cl₂ (225.29 mg, 1.669 mmol, 1.5 equiv) in portions at −78° C. The resulting mixture was stirred for 2 h at −30° C. The resulting mixture was concentrated under reduced pressure. The residue was purified by Pre-TLC DCM/MeOH (30:1) to afford 13-6 (180 mg, 36.76%) as a white solid. LCMS (ESI, m/z): [M+H]⁺=440

### Step G. N-(3-{4-[1-(2,6-Dioxopiperidin-3-yl)-3-methyl-2-oxo-1,3-benzodiazol-4-yl]piperidin-1-yl}propyl)-N-methyl-4-{7′-[(1R,2S)-2-methylcyclopentyl]-6′-oxospiro[cyclopropane-1,5′-pyrrolo[2,3-d]pyrimidin]-2′-ylamino}piperidine-1-sulfonamide (13)

To a stirred solution of 13-5 (60 mg, 0.145 mmol, 1 equiv) in DCM (0.6 mL) was added 13-6 (89.37 mg, 0.203 mmol, 1.4 equiv) and TEA (44.05 mg, 0.435 mmol, 3 equiv). The resulting mixture was stirred for additional 1 h at 40° C. The resulting mixture was concentrated under reduced pressure. The residue was purified by reverse phase flash to afford compound 13 (45.4 mg, 34.40%) as a white solid.

### Example 14. 3-(3-Methyl-4-{1-[(1-{4-[7′-(2-methylcyclopentyl)-6′-oxospiro[cyclopropane-1,5′-pyrrolo[2,3-d]pyrimidin]-2′-ylamino]piperidin-1-ylsulfonyl}piperidin-4-yl)methyl]piperidin-4-yl}-2-oxo-1,3-benzodiazol-1-yl)piperidine-2,6-dione (14)

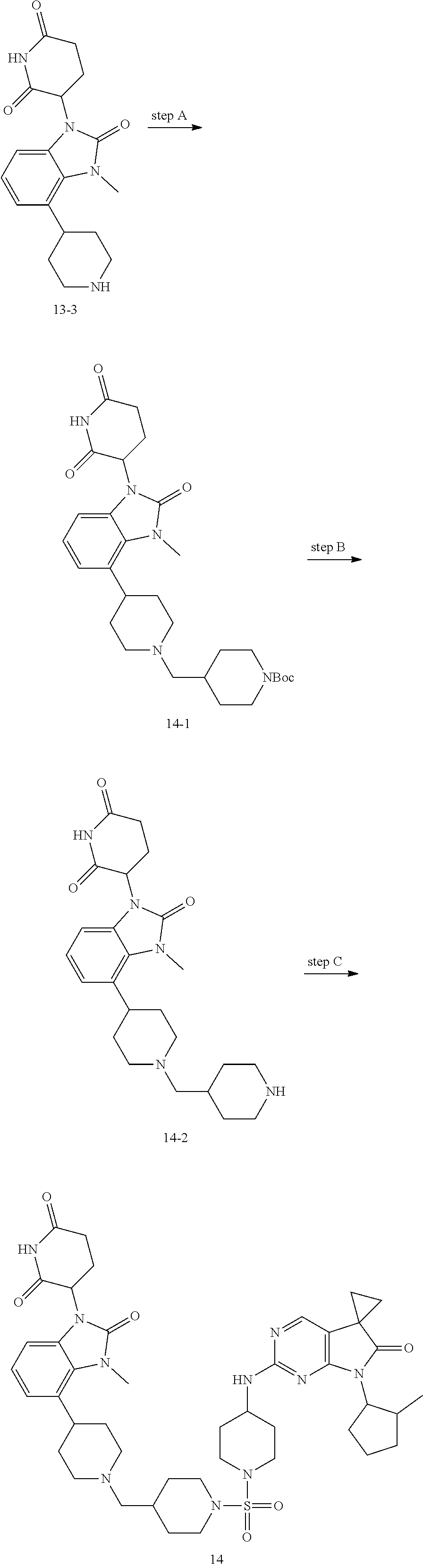 [SMILES: Cn1c(=O)n(C2CCC(=O)NC2=O)c2cccc(C3CCNCC3)c21 ; Cn1c(=O)n(C2CCC(=O)NC2=O)c2cccc(C3CCN(CC4CCN(C(=O)OC(C)(C)C)CC4)CC3)c21 ; Cn1c(=O)n(C2CCC(=O)NC2=O)c2cccc(C3CCN(CC4CCNCC4)CC3)c21 ; CC1CCCC1N1C(=O)C2(CC2)c2cnc(NC3CCN(S(=O)(=O)N4CCC(CN5CCC(c6cccc7c6n(C)c(=O)n7C6CCC(=O)NC6=O)CC5)CC4)CC3)nc21]

### Step A. Tert-butyl 4-({4-[1-(2,6-dioxopiperidin-3-yl)-3-methyl-2-oxo-1,3-benzodiazol-4-yl]piperidin-1-yl}methyl)piperidine-1-carboxylate (14-1)

To a solution of 13-3 (600 mg, 1.752 mmol, 1 equiv) and tert-butyl 4-(bromomethyl)piperidine-1-carboxylate (974.97 mg, 3.504 mmol, 2 equiv) in MeCN (5 mL) were added NaI (262.67 mg, 1.752 mmol, 1 equiv) and K₂CO₃ (1453.09 mg, 10.512 mmol, 6 equiv) at room temperature. The resulting mixture was stirred for overnight at 70° C. The residue was purified by Prep-TLC (CH₂Cl₂/MeOH 30:1) to afford 14-1 (350 mg, 37.01%) as a yellow solid. LCMS (ESI, m/z): [M+H]⁺=540

### Step B. 3-{3-Methyl-2-oxo-4-[1-(piperidin-4-ylmethyl)piperidin-4-yl]-1,3-benzodiazol-1-yl}piperidine-2,6-dione (14-2)

To a stirred mixture of 14-1 (65 mg, 0.120 mmol, 1 equiv) in ACN (5 mL) was added 4M HCl (gas) in 1,4-dioxane (1 mL). The resulting mixture was stirred for 1 h at 25° C. The resulting mixture was concentrated under reduced pressure to afford 14-2 (60 mg, crude) as a white solid. The crude product was used in the next step directly without further purification. LCMS (ESI, m/z): [M+H]⁺=440.

### Step C. 3-(3-Methyl-4-{1-[(1-{4-[7′-(2-methylcyclopentyl)-6′-oxospiro[cyclopropane-1,5′-pyrrolo[2,3-d]pyrimidin]-2′-ylamino]piperidin-1-ylsulfonyl}piperidin-4-yl)methyl]piperidin-4-yl}-2-oxo-1,3-benzodiazol-1-yl)piperidine-2,6-dione (14)

To a stirred solution of 14-2 (60 mg, 0.137 mmol, 1 equiv) in DCM (0.6 mL) was added 13-6 (72.07 mg, 0.164 mmol, 1.2 equiv) and TEA (41.44 mg, 0.411 mmol, 3 equiv). The resulting mixture was stirred for 1 h at 40 degrees C. The resulting mixture was concentrated under reduced pressure. The residue was purified by reverse phase flash Pre-HPLC to afford compound 14 (19.1 mg, 15.26%) as a white solid.

### Example 22. 4-(1-(2,6-dioxopiperidin-3-yl)-3-methyl-2-oxo-2,3-dihydro-1H-benzo[d]imidazol-4-yl)-N-methyl-N-(1-((4-((7′-(2-methylcyclopentyl)-6′-oxo-6′,7′-dihydrospiro[cyclopropane-1,5′-pyrrolo[2,3-d] pyrimidin]-2′-yl)amino)piperidin-1-yl)sulfonyl)azetidin-3-yl)butanamide (22)

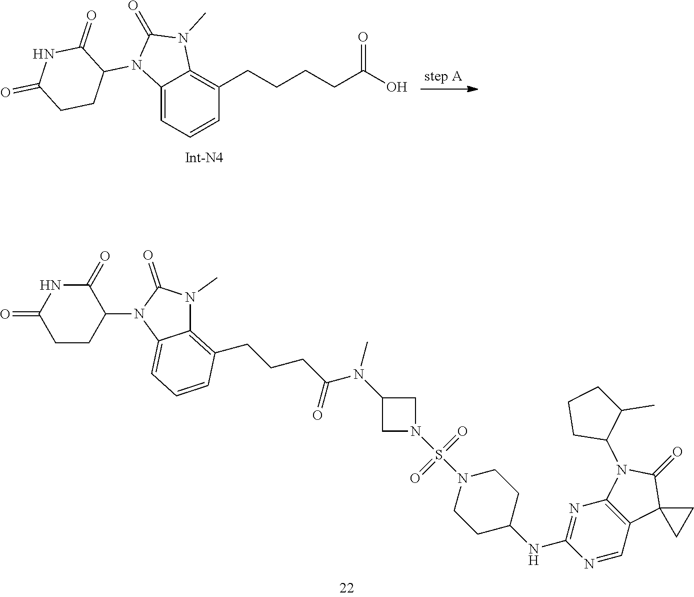 [SMILES: Cn1c(=O)n(C2CCC(=O)NC2=O)c2cccc(CCCCC(=O)O)c21 ; CC1CCCC1N1C(=O)C2(CC2)c2cnc(NC3CCN(S(=O)(=O)N4CC(N(C)C(=O)CCCc5cccc6c5n(C)c(=O)n6C5CCC(=O)NC5=O)C4)CC3)nc21]

### Step A. 4-(1-(2,6-dioxopiperidin-3-yl)-3-methyl-2-oxo-2,3-dihydro-1H-benzo[d]imidazol-4-yl)-N-methyl-N-(1-((4-((7′-(2-methylcyclopenlyl)-6′-oxo-6′,7′-dihydrospiro[cyclopropane-1,5′-pyrrolo[2,3-d]pyrimidin]-2′-yl)amino)piperidin-1-yl)sulfonyl)azetidin-3-yl)butanamide (22)

Into a 8 mL vial were added Int-10 (50 mg, 0.102 mmol, 1 equiv), Int-N4 (52.90 mg, 0.153 mmol, 1.5 equiv), EDCI (29.36 mg, 0.153 mmol, 1.5 equiv), DIPEA (131.98 mg, 1.020 mmol, 10 equiv), 1-lambda4-pyridine-1,2-diol (17.17 mg, 0.153 mmol, 1.5 equiv) and MeCN (2 mL) at room temperature. The resulting mixture was stirred for 1 h at 40° C. under nitrogen atmosphere. Desired product could be detected by LCMS. The resulting mixture was diluted with water (2 mL). The resulting mixture was extracted with EtOAc (5 mL). The combined organic layers were dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (10 mmol/L NH₄HCO₃), 30% to 50% gradient in 35 min; detector, UV 254 nm. This resulted in 4-[1-(2,6-dioxopiperidin-3-yl)-3-methyl-2-oxo-1,3-benzodiazol-4-yl]-N-methyl-N-(1-{4-[7′-(2-methylcyclopentyl)-6′-oxospiro[cyclopropane-1,5′-pyrrolo[2,3-d]pyrimidin]-2′-ylamino]piperidin-1-ylsulfonyl}azetidin-3-yl) butanamide (22, 14.8 mg, 17.10%) as a white solid. LCMS (ESI, m/z): [M+H]⁺ 817.4.

### Example 116. N-(3-(4-(1-(2,6-dioxopiperidin-3-yl)-3-methyl-2-oxo-2,3-dihydro-1H-benzo[d]imidazol-4-yl)piperidin-1-yl)propyl)-4-((7′-((1R,3R)-3-hydroxycyclohexyl)-6′-oxo-6′,7′-dihydrospiro[cyclopropane-1,5′-pyrrolo[2,3-d]pyrimidin]-2′-yl)amino)-N-methylbenzenesulfonamide (116)

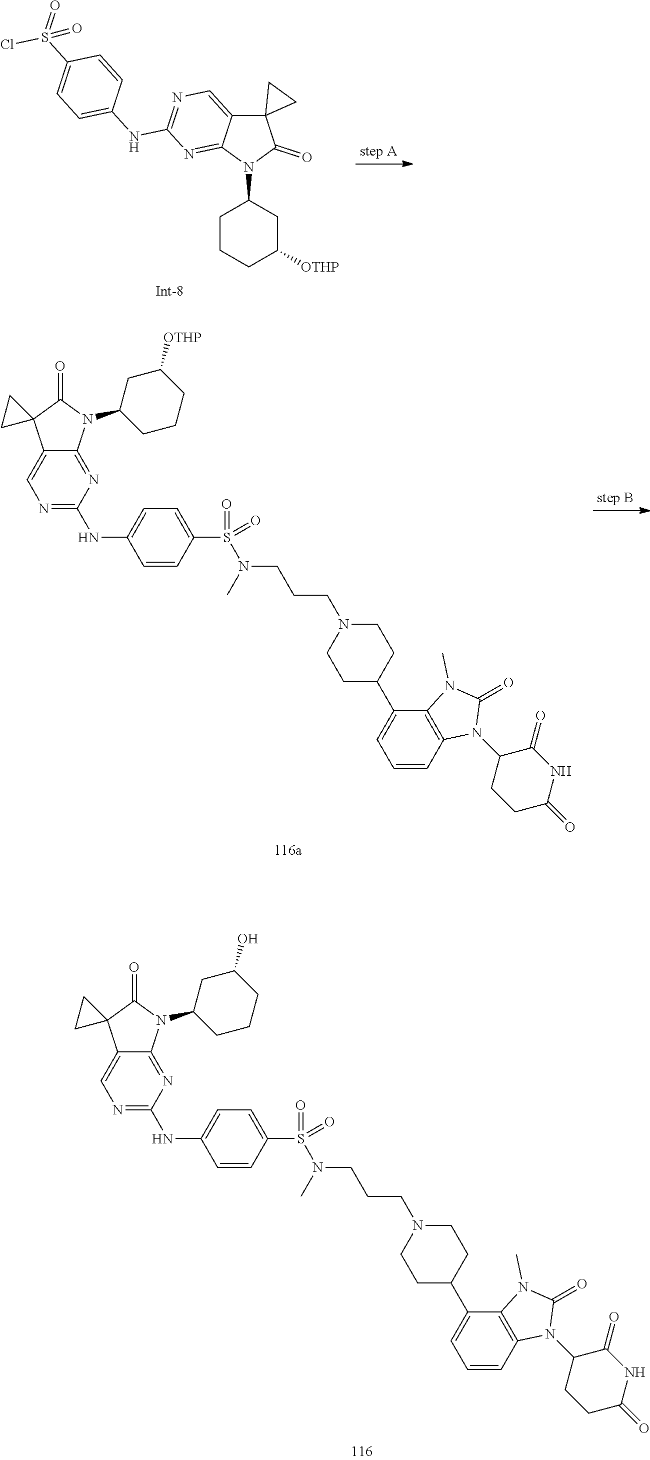

### Step A. N-(3-(4-(1-(2,6-dioxopiperidin-3-yl)-3-methyl-2-oxo-2,3-dihydro-1H-benzo[d]imidazol-4-yl)piperidin-1-yl)propyl)-N-methyl-4-((6′-oxo-7′-((1R,3R)-3-((tetrahydro-2H-pyran-2-yl)oxy)cyclo hexyl)-6′,7′-dihydrospiro[cyclopropane-1,5′-pyrrolo[2,3-d]pyrimidin]-2′-yl)amino)benzenesulfonamide (116a)

To a stirred solution of Int-8 (200 mg, 0.375 mmol, 1 equiv) and TEA (113.90 mg, 1.125 mmol, 3 equiv) in DCM (0.6 mL) was added 13-5 (155.16 mg, 0.375 mmol, 1 equiv) dropwise at room temperature. The resulting mixture was stirred for 1 h at 30° C. Desired product could be detected by LCMS. The resulting mixture was concentrated under reduced pressure. The residue was purified by silica gel column chromatography, eluted with CH₂Cl₂/MeOH (10:1) to afford 116a (56 mg, 9.77%) as a yellow solid. LCMS (ESI, m/z): [M+H]⁺=910.4

### Step B. N-(3-{4-[1-(2,6-dioxopiperidin-3-yl)-3-methyl-2-oxo-1,3-benzodiazol-4-yl]piperidin-1-yl}propyl)-4-{7′-[(1R,3R)-3-hydroxycyclohexyl]-6′-oxospiro[cyclopropane-1,5′-pyrrolo[2,3-d]pyrimidin]-2′-ylamino}-N-methylbenzenesulfonamide (116)

To a stirred solution of 116a (50 mg, 0.055 mmol, 1 equiv) in CH₃CN (2.5 mL) was added HCl (gas) in 1,4-dioxane (0.5 mL) dropwise at 0° C. The resulting mixture was stirred for 30 min at 0° C. Mainly product could be detected by LCMS. The mixture was basified to pH 7 with NH₃H₂O. The resulting mixture was concentrated under reduced pressure. The crude product (50 mg) was purified by Prep-HPLC with the following conditions (Column: Kinetex EVO C18 Column, 30*150 mm, 5 μm; Mobile Phase A: Water (10 mmol/L NH₄HCO₃), Mobile Phase B: MeCN; Flow rate: 60 mL/min mL/min; Gradient: 25% B to 40% B in 10 min; Wave Length: 254 nm/220 nm nm; RT1 (min): 9.1) to afford N-(3-{4-[1-(2,6-dioxopiperidin-3-yl)-3-methyl-2-oxo-1,3-benzodiazol-4-yl]piperidin-1-yl}propyl)-4-{7′-[(1R,3R)-3-hydroxy cyclohexyl]-6′-oxospiro[cyclopropane-1,5′-pyrrolo[2,3-d]pyrimidin]-2′-ylamino}-N-methylbenzenesulfonamide (116, 11.6 mg, 24.44%) as a white solid. LCMS (ESI, m/z): [M+H]⁺=826.2

### Example 117. N-(3-(4-(1-(2,6-dioxopiperidin-3-yl)-3-methyl-2-oxo-2,3-dihydro-1H-benzo[d]imidazol-4-yl)piperidin-1-yl)propyl)-N-methyl-4-((7′-(2-methylcyclopentyl)-6′-oxo-6′,7′-dihydrospiro [cyclopropane-1,5′-pyrrolo[2,3-d]pyrimidin]-2′-yl)amino)benzenesulfonamide (117)

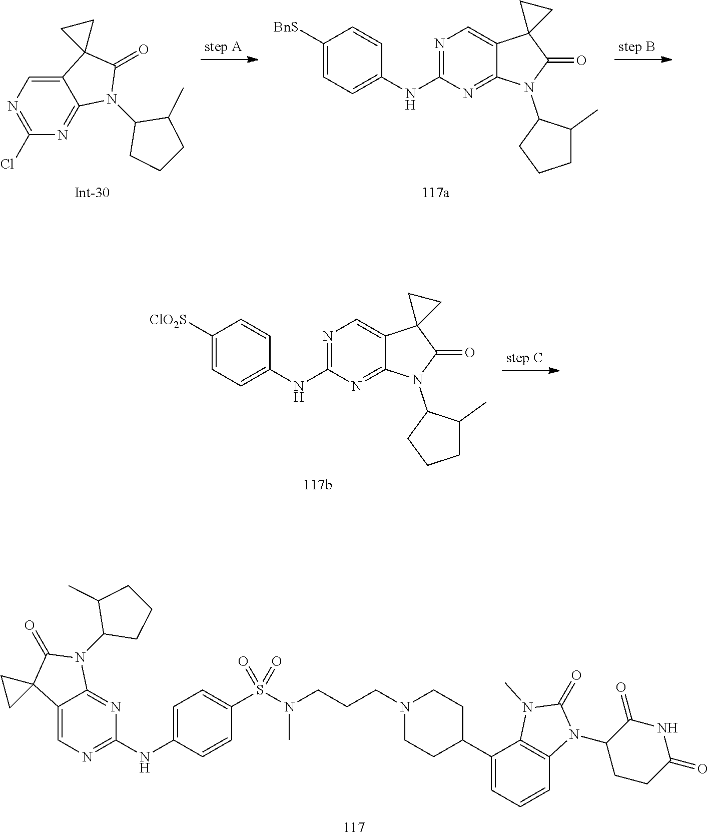

### Step A. 2′-((4-(benzylthio)phenyl)amino)-7′-(2-methylcyclopentyl)spiro [cyclopropane-1,5′-pyrrolo[2,3-d]pyrimidin]-6′(7′H)-one (117a)

To a stirred solution of 4-(benzylsulfanyl)aniline (200 mg, 0.929 mmol, 1.5 equiv) and Int-30 (172.00 mg, 0.619 mmol, 1 equiv) in IPA (4 mL) was added TFA (706.10 mg, 6.193 mmol, 10 equiv) dropwise at room temperature. The resulting mixture was stirred for 5 h at 80° C. Desired product could be detected by LCMS. The mixture was basified to pH 7 with saturated NaHCO₃ (aq.). The aqueous layer was extracted with EtOAc (3×50 mL). The resulting mixture was concentrated under reduced pressure. The residue was purified by silica gel column chromatography, eluted with petroleum ether/EtOAc (5/1) to afford 117a (310 mg, 94.7%) as a white solid. LCMS (ESI, m/z): [M+H]⁺=457.2

### Step B. 4-[7′-(2-methylcyclopentyl)-6′-oxospiro[cyclopropane-1,5′-pyrrolo[2,3-d]pyrimidin]-2′-ylamino]benzenesulfonyl chloride (117b)

To a stirred solution of 117a (150 mg, 0.346 mmol, 1 equiv) in DCM (4.5 mL), HOAc (83.23 mg, 1.384 mmol, 4 equiv) and H₂O (25.59 mg, 1.419 mmol, 4.1 equiv) was added SO₂Cl₂ (191.72 mg, 1.419 mmol, 4.1 equiv) dropwise at 0° C. The resulting mixture was stirred for 30 min at 0° C. Desired product could be detected by LCMS. The mixture was basified to pH 7 with saturated NaHCO₃ (aq.). The aqueous layer was extracted with EtOAc (3×10 mL). The resulting mixture was concentrated under reduced pressure. The residue was purified by silica gel column chromatography, eluted with petroleum ether/EtOAc (5/1) to afford 117b (140 mg, 84.86%) as a white solid. LCMS (ESI, m/z): [M+H]⁺=433.1

### Step C. N-(3-{4-[1-(2,6-dioxopiperidin-3-yl)-3-methyl-2-oxo-1,3-benzodiazol-4-yl]piperidin-1-yl}propyl)-N-methyl-4-[7′-(2-methylcyclopentyl)-6′-oxospiro[cyclopropane-1,5′-pyrrolo[2,3-d]pyrimidin]-2′-ylamino]benzenesulfonamide (117)

To a stirred solution of 13-5 (90 mg, 0.218 mmol, 1 equiv) in DCM (1.8 mL) was added 117b (113.07 mg, 0.262 mmol, 1.2 equiv) and Et₃N (66.07 mg, 0.654 mmol, 3 equiv) dropwise at room temperature. The resulting mixture was stirred for 1 h at 30° C. Desired product could be detected by LCMS. The resulting mixture was concentrated under reduced pressure. The crude product (90 mg) was purified by Prep-HPLC with the following conditions (Column: Xbridge Phenyl OBD Column, 19*150 mm, 5 m; Mobile Phase A: Water (10 mmol/L NH₄HCO₃+0.05% NH₃·H₂O, Mobile Phase B: ACN; Flow rate: 60 mL/min mL/min; Gradient: 40% B to 50% B in 8 min; Wave Length: 254 nm/220 nm nm; RT1 (min): 9.85) to afford N-(3-{4-[1-(2,6-dioxopiperidin-3-yl)-3-methyl-2-oxo-1,3-benzodiazol-4-yl]piperidin-1-yl}propyl)-N-methyl-4-[7′-(2-methylcyclopentyl)-6′-oxospiro[cyclopropane-1,5′-pyrrolo[2,3-d]pyrimidin]-2′-ylamino]benzenesulfonamide (117, 46.0 mg, 25.36%) as a white solid. LCMS (ESI, m/z): [M+H]⁺=810.3

### Example 118. 1-(6-(1-(3-((4-((7′-((1R,3R)-3-hydroxycyclohexyl)-6′-oxo-6′,7′-dihydrospiro[cyclopropane-1,5′-pyrrolo[2,3-d]pyrimidin]-2′-yl)amino)piperidin-1-yl)sulfonyl)benzyl)piperidin-4-yl)-1-methyl-1H-indazol-3-yl)dihydropyrimidine-2,4(1H,3H)-dione (118)

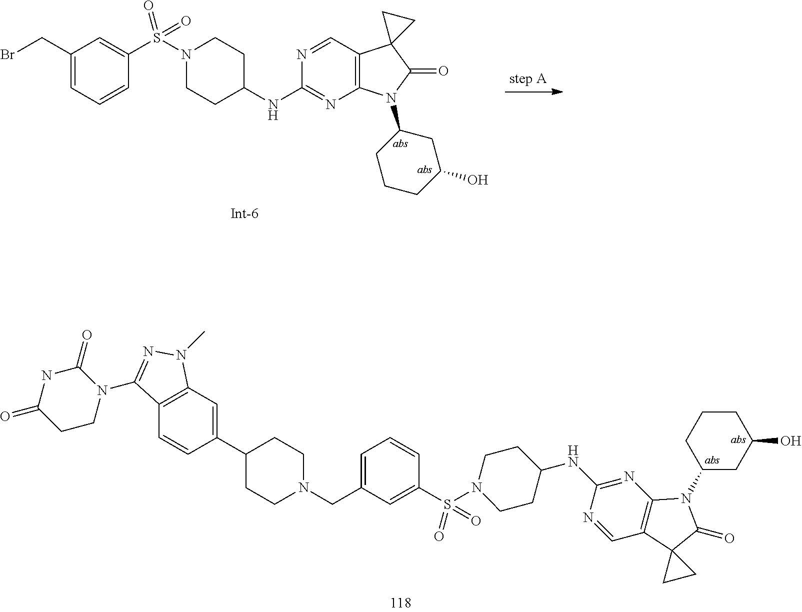 [SMILES: O=C1N([C@@H]2CCC[C@@H](O)C2)c2nc(NC3CCN(S(=O)(=O)c4cccc(CBr)c4)CC3)ncc2C12CC2 ; Cn1nc(N2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)c4)CC3)cc21]

### Step A. 1-(6-(1-(3-((4-((7′-((1R,3R)-3-hydroxycyclohexyl)-6′-oxo-6′,7′-dihydrospiro[cyclopropane-1,5′-pyrrolo[2,3-d]pyrimidin]-2′-yl)amino)piperidin-1-yl)sulfonyl)benzyl)piperidin-4-yl)-1-methyl-1H-indazol-3-yl)dihydropyrimidine-2,4(1H,3H)-dione (118)

To a stirred solution of Int-24 (30.49 mg, 0.094 mmol, 1.1 equiv) and KI (7.03 mg, 0.043 mmol, 0.5 equiv) in CH₃CN (2 mL) was added K₂CO₃ (35.10 mg, 0.255 mmol, 3 equiv) and Int-6 (50 mg, 0.085 mmol, 1 equiv) in portions at room temperature. The resulting mixture was stirred for 1 h at 30° C. Desired product could be detected by LCMS. The resulting mixture was concentrated under reduced pressure. The crude product (50 mg) was purified by Prep-HPLC with the following conditions (Column: XBridge Prep phenyl Column, 30*150 mm, 5 μm; Mobile Phase A: Water (10 mmol/L NH₄HCO₃+0.05% NH₃·H₂O), Mobile Phase B: ACN; Flow rate: 60 mL/min mL/min; Gradient: 30% B to 50% B in 10 min; Wave Length: 254 nm/220 nm; RT1 (min): 9.87) to afford 1-[6-(1-{[3-(4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl)phenyl]methyl}piperidin-4-yl)-1-methylindazol-3-yl]-1,3-diazinane-2,4-dione (118, 7.9 mg, 11.05%) as a white solid. LCMS (ESI, m/z): [M+H]⁺=837.4

### Example 119. 3-{5-[4-({4-[3-(4-{7′-[(1R,3R)-3-hydroxycyclohexyl]-6′-oxospiro[cyclopropane-1,5′-pyrrolo [2,3-d]pyrimidin]-2′-ylamino}piperidin-1-ylsulfonyl)phenyl]piperazin-1-yl}methyl)piperidin-1-yl]-1-oxo-3H-isoindol-2-yl}piperidine-2,6-dione (119)

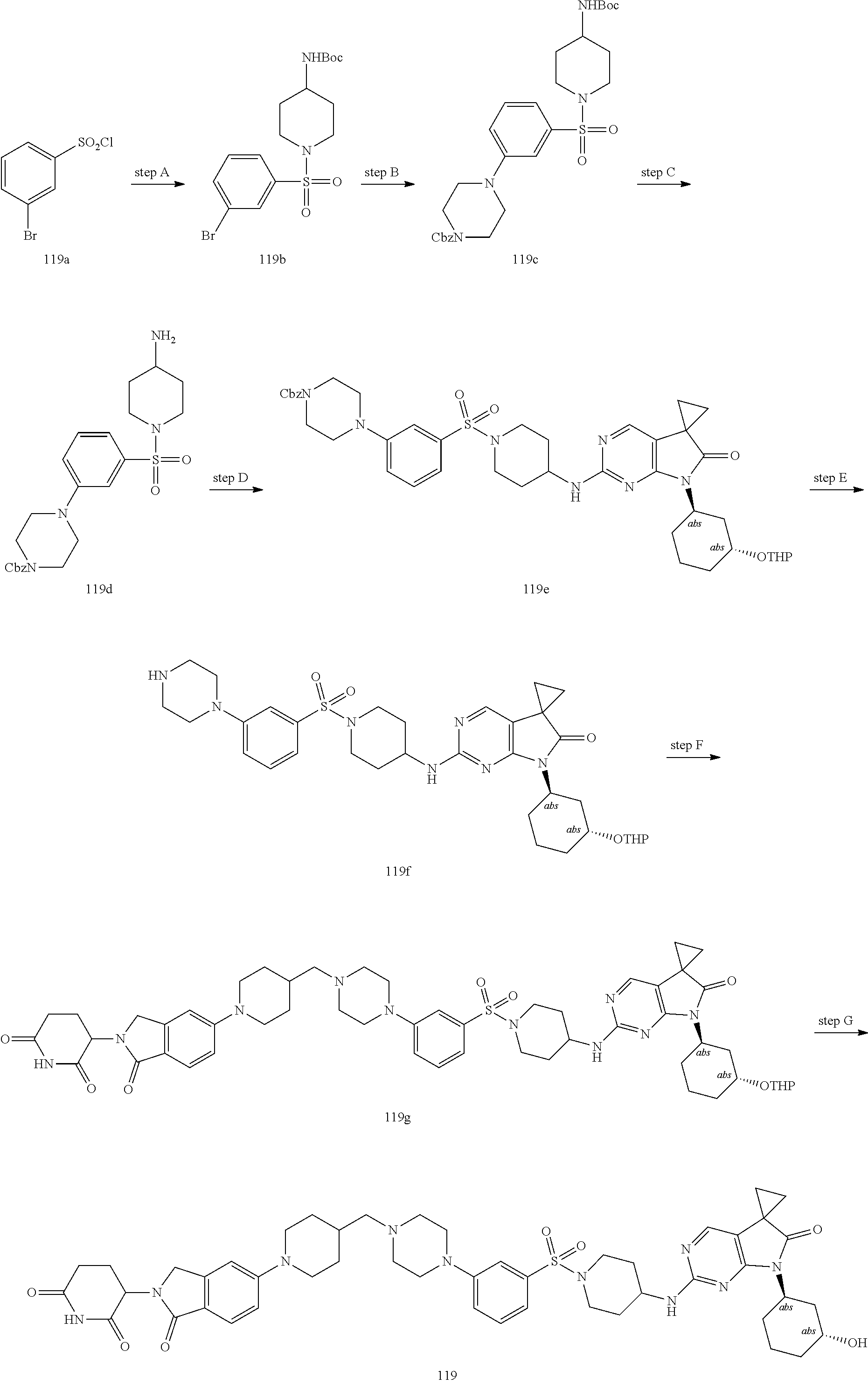

### Step A. tert-butyl N-[l-(3-bromobenzenesulfonyl)piperidin-4-yl]carbamate (119b)

To a stirred mixture of 119a (10 g, 49.930 mmol, 1.00 equiv) and Et₃N (15.16 g, 149.790 mmol, 3 equiv) in THF (100 mL) was added 3-bromobenzenesulfonyl chloride (15.31 g, 59.916 mmol, 1.2 equiv). The resulting mixture was stirred for 1 h at 25° C. under air atmosphere. Desired product could be detected by LCMS. The resulting mixture was concentrated under reduced pressure. The residue was purified by silica gel column chromatography, eluted with EtOAc/petroleum ether (1/10) to afford 119b (16 g, 69.69%) as a white solid. LCMS (ESI, m/z): [M+H]⁺=419.1

### Step B. benzyl 4-(3-{4-[(tert-butoxycarbonyl)amino]piperidin-1-ylsulfonyl}phenyl)piperazine-1-carboxylate (119c)

To a stirred mixture of 119b (2 g, 4.770 mmol, 1 equiv) and benzyl piperazine-1-carboxylate (1.58 g, 7.155 mmol, 1.5 equiv) in DMSO (20 mL) were added K₂CO₃ (1.98 g, 14.310 mmol, 3 equiv), CuI (0.18 g, 0.954 mmol, 0.2 equiv) and (2S)-pyrrolidine-2-carboxylic acid (109.82 mg, 0.954 mmol, 0.2 equiv). The resulting mixture was stirred for 3 h at 90° C. under nitrogen atmosphere. Desired product could be detected by LCMS. The reaction was quenched by the addition of water (10 mL). The resulting mixture was extracted with EtOAc. The combined organic layers were dried over anhydrous Na₂SO₄. After filtration, the filtrate was concentrated under reduced pressure. The residue was purified by silica gel column chromatography, eluted with EtOAc/petroleum ether (1/1) to afford benzyl 119c (665 mg, 19.79%) as a white solid. LCMS (ESI, m/z): [M+H]⁺=559.3

### Step C. bis(benzyl 4-[3-(4-aminopiperidin-1-ylsulfonyl)phenyl]piperazine-1-carboxylate) (119d)

To a stirred mixture of 119c (650 mg, 0.582 mmol, 1 equiv) in MeCN (30 mL) was added HCl (gas) in 1,4-dioxane (5 mL). The resulting mixture was stirred for 0.5 h at 0° C. under nitrogen atmosphere. Desired product could be detected by LCMS. The resulting mixture was concentrated under reduced pressure to afford 105d (600 mg, crude) as a white solid. The crude product of 119d was used in the next step directly without further purification. LCMS (ESI, m/z): [M+H]⁺=459.2

### Step D. benzyl 4-[3-(4-{7′-[(1R,3R)-3-(oxan-2-yloxy)cyclohexyl]-6′-oxospiro[cyclopropane-1,5′-pyrrolo[2,3-d]pyrimidin]-2′-ylamino}piperidin-1-ylsulfonyl)phenyl]piperazine-1-carboxylate (119e)

To a stirred mixture of benzyl 119d (600 mg, 1.308 mmol, 1 equiv) and Int-5 (593.28 mg, 1.570 mmol, 1.2 equiv) in dioxane (7 mL) were added RuPhos Palladacycle G3 (218.86 mg, 0.262 mmol, 0.2 equiv) and BINAP (81.47 mg, 0.131 mmol, 0.1 equiv) and Cs₂CO₃ (1278.89 mg, 3.924 mmol, 3 equiv) under nitrogen atmosphere. The resulting mixture was stirred for 12 h at 90° C. Desired product could be detected by LCMS. The resulting mixture was concentrated under reduced pressure. The residue was purified by silica gel column chromatography, eluted with DCM/MeOH (20:1) to afford 119e (350 mg, 31.67%) as a white solid. LCMS (ESI, m/z): [M+H]⁺=800.4

### Step E 7′-[(1R,3R)-3-(oxan-2-yloxy)cyclohexyl]-2′-({1-[3-(piperazin-1-yl)benzenesulfonyl]piperidin-4-yl}amino)spiro[cyclopropane-1,5′-pyrrolo[2,3-d]pyrimidin]-6′-one (119f)

To a stirred solution of 119e (300 mg, 0.375 mmol, 1 equiv) in THF (5 mL) was added Pd/C (59.86 mg, 0.563 mmol, 1.50 equiv) and Pd(OH)₂/C (60.03 mg, 0.427 mmol, 1.14 equiv) under nitrogen atmosphere in a 25 mL round-bottom flask. The mixture was hydrogenated at room temperature for 12 h under hydrogen atmosphere using a hydrogen balloon. Desired product could be detected by LCMS. The resulting mixture was filtered, the filter cake was washed with DCM. The filtrate was concentrated under reduced pressure to afford 119f (250 mg, crude) as a white solid. LCMS (ESI, m/z): [M+H]⁺=666.3

### Step F. 3-{5-[4-({4-[3-(4-{7′-[(1R,3R)-3-(oxan-2-yloxy)cyclohexyl]-6′-oxospiro[cyclopropane-1,5′-pyrrolo[2,3-d]pyrimidin]-2′-ylamino}piperidin-1-ylsulfonyl)phenyl]piperazin-1-yl}methyl)piperidin-1-yl]-1-oxo-3H-isoindol-2-yl}piperidine-2,6-dione (119g)

To a stirred mixture of 119f (75 mg, 0.113 mmol, 1 equiv) and Int-28 (40.03 mg, 0.113 mmol, 1 equiv) in DMF (2 mL) and HOAc (2.03 mg, 0.034 mmol, 0.3 equiv) was added NaBH₃CN (21.23 mg, 0.339 mmol, 3 equiv). The resulting mixture was stirred for 1 h at 25° C. under air atmosphere. Desired product could be detected by LCMS. The reaction was quenched by the addition of water (5 mL). The resulting mixture was extracted with EA. The combined organic layers were dried over anhydrous Na₂SO₄. After filtration, the filtrate was concentrated under reduced pressure. The residue was purified by silica gel column chromatography, eluted with petroleum ether/EtOAc (1:1) to afford 119g (50 mg, 41.07%) as a brown solid. LCMS (ESI, m/z): [M+H]⁺=1005.5

### Step G. 3-{5-[4-({4-[3-(4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl)phenyl]piperazin-1-yl}methyl)piperidin-1-yl]-1-oxo-3H-isoindol-2-yl}piperidine-2,6-dione (119)

To a stirred mixture of 119g (45 mg, 0.045 mmol, 1 equiv) in ACN (1 mL) was added HCl (gas) in 1,4-dioxane (0.5 mL). The resulting mixture was stirred for 0.5 h at 0° C. under air atmosphere. Desired product could be detected by LCMS. The resulting mixture was concentrated under reduced pressure. The resulting mixture was diluted with DCM and was neutralized with Et3N. The resulting mixture was concentrated under vacuum. The crude product (45 mg) was purified by Prep-HPLC with the following conditions (Column: Xbridge Phenyl OBD Column, 19*150 mm, 5 m; Mobile Phase A: Water (10 mmol/L NH₄HCO₃+0.05% NH₃H₂0, Mobile Phase B: ACN; Flow rate: 60 mL/min mL/min; Gradient: 45% B to 60% B in 10 min; Wave Length: 254 nm/220 nm nm; RT1 (min): 10.15) to afford 3-{5-[4-({4-[3-(4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl)phenyl]piperazin-1-yl}methyl)piperidin-1-yl]-1-oxo-3H-isoindol-2-yl}piperidine-2,6-dione (119, 12.9 mg, 30.47%) as a white solid. LCMS (ESI, m/z): [M+H]⁺=921.4

### Example 121. 3-(6-fluoro-5-(4-(3-((4-((7′-((1R,3R)-3-hydroxycyclohexyl)-6′-oxo-6′,7′-dihydrospiro [cyclopropane-1,5′-pyrrolo[2,3-d]pyrimidin]-2′-yl)amino)piperidin-1-yl)sulfonyl)benzyl) piperazin-1-yl)-1-oxoisoindolin-2-yl)piperidine-2,6-dione (121)

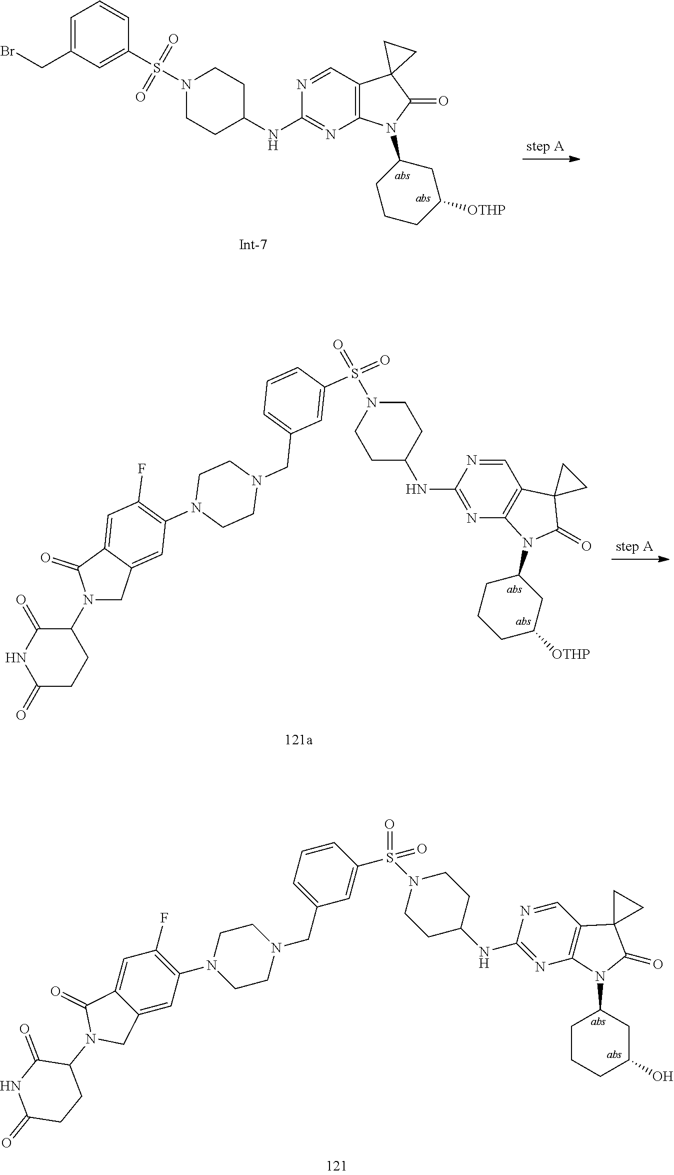

### Step A. 3-(6-fluoro-1-oxo-5-(4-(3-((4-((6′-oxo-7′-((1R,3R)-3-((tetrahydro-2H-pyran-2-yl)oxy)cyclohexyl)-6′,7′-dihydrospiro[cyclopropane-1,5′-pyrrolo[2,3-d]pyrimidin]-2′-yl)amino) piperidin-1-yl)sulfonyl) benzyl)piperazin-1-yl)isoindolin-2-yl)piperidine-2,6-dione (121a)

Into a 8 mL vial were added Int-26 (80 mg, 0.231 mmol, 1 equiv), Int-7 (171.41 mg, 0.254 mmol, 1.10 equiv), K₂CO₃ (95.76 mg, 0.693 mmol, 3.00 equiv), KI (19.17 mg, 0.115 mmol, 0.50 equiv) and ACN (2 mL) at room temperature. The resulting mixture was stirred for 1 h at 80° C. under nitrogen atmosphere. Desired product could be detected by LCMS. The resulting mixture was diluted with water (2 mL). The resulting mixture was extracted with EtOAc (3×2 mL). The combined organic layers were dried over anhydrous Na₂SO₄. After filtration, the filtrate was concentrated under reduced pressure. The residue was purified by Prep-TLC (CH₂Cl₂/MeOH 15:1) to afford 121a (62 mg, 26.13%) as a white solid. LCMS (ESI, m/z): [M−H]⁺ 940.4.

### Step B. 3-[6-fluoro-5-(4-{[3-(4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl)phenyl]methyl}piperazin-1-yl)-1-oxo-3H-isoindol-2-yl]piperidine-2,6-dione (121)

To a stirred solution of 121a (58 mg, 0.062 mmol, 1 equiv) in MeCN (1 mL) was added HCl (gas) in 1,4-dioxane (0.2 mL, 6.583 mmol, 106.70 equiv) dropwise at 0° C. under nitrogen atmosphere. The resulting mixture was stirred for 1 h at 0° C. under nitrogen atmosphere. Desired product could be detected by LCMS. The mixture was neutralized to pH 7 with TEA. The resulting mixture was concentrated under reduced pressure. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. This resulted in 3-[6-fluoro-5-(4-{[3-(4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl)phenyl]methyl}piperazin-1-yl)-1-oxo-3H-isoindol-2-yl]piperidine-2,6-dione (121, 23.4 mg, 42.67%) as a white solid. LCMS (ESI, m/z): [M−H]⁺ 856.4.

### Example 122. 3-{5-[4-({1-[3-(4-{7′-[(1R,3R)-3-hydroxycyclohexyl]-6′-oxospiro[cyclopropane-1,5′-pyrrolo [2,3-d]pyrimidin]-2′-ylamino}piperidin-1-ylsulfonyl)phenyl]piperidin-4-yl}methyl)piperazin-1-yl]-1-oxo-3H-isoindol-2-yl}piperidine-2,6-dione (122)

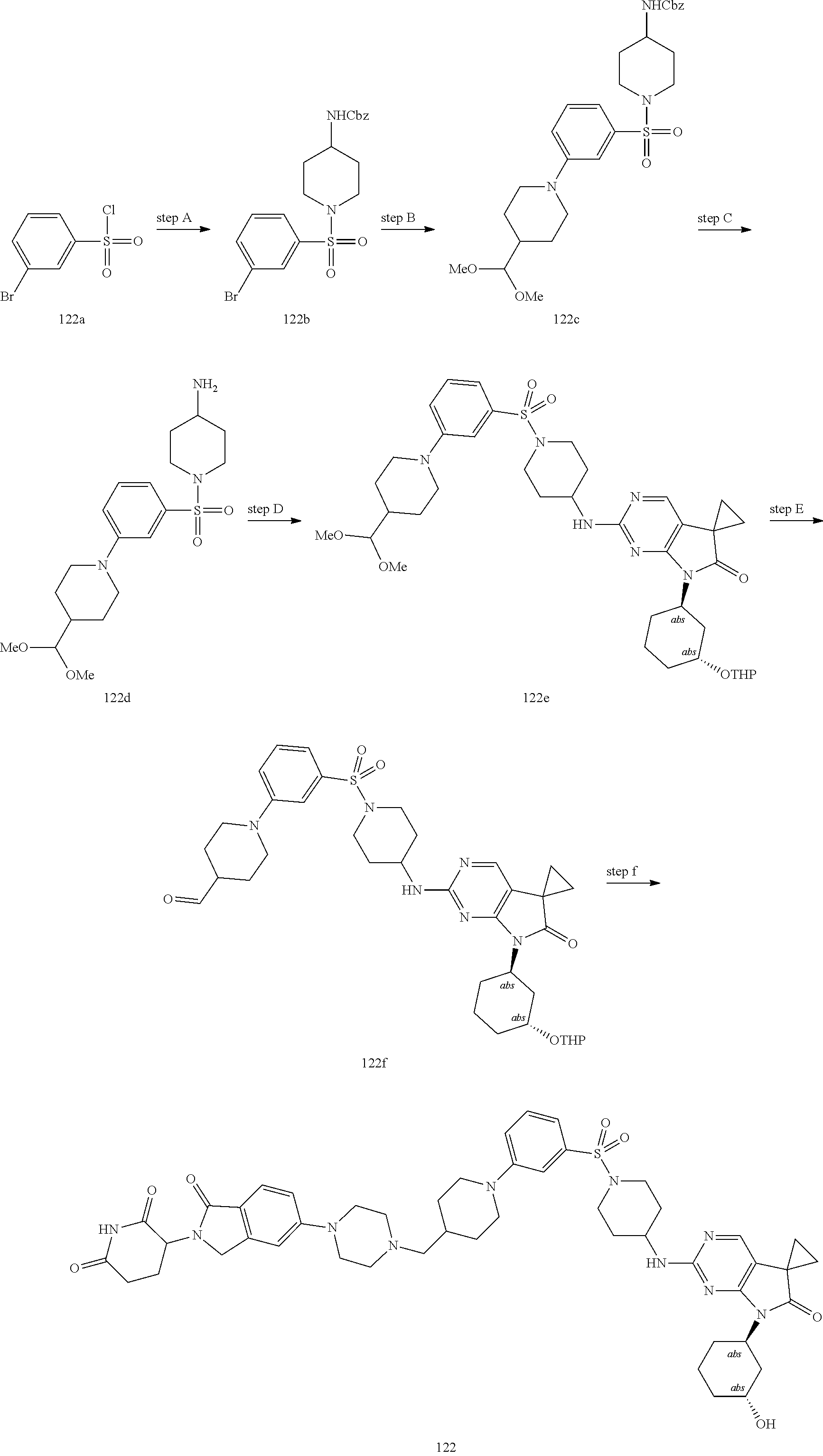

### Step A. benzyl N-[1-(3-bromobenzenesulfonyl)piperidin-4-yl]carbamate (122b)

To a stirred solution of 122a (2 g, 8.536 mmol, 1.00 equiv) in anhydrous THF (40 mL) was added 3-bromobenzenesulfonyl chloride (2.29 g, 8.963 mmol, 1.05 equiv) at 0° C. and stirred for 2 h at 25° C. The reaction progress was monitored by LCMS. Desired product could be detected by LCMS. The reaction was quenched by the addition of water (50 ml) at 25° C. The resulting mixture was extracted with DCM (2×5 ml). The combined organic layers were dried over anhydrous Na₂SO₄. After filtration, the filtrate was concentrated under reduced pressure. The residue was purified by trituration with PE (50 ml). The precipitated solids were collected by filtration and washed with PE (3×50 ml). This resulted in 122b (3.5 g, 90.44%) as a white solid. LCMS (ESI, m/z): [M+H]⁺ 453.0

### Step B. benzyl N-(1-{3-[4-(dimethoxymethyl)piperidin-1-yl]benzenesulfonyl}piperidin-4-yl)carbamate (122c)

To a solution of benzyl 122b (2 g, 4.412 mmol, 1 equiv) and 4-(dimethoxymethyl)piperidine (842.95 mg, 5.294 mmol, 1.2 equiv) in Toluene (40 mL) were added Cs₂CO₃ (4312.15 mg, 13.236 mmol, 3 equiv) and Pd₂(dba)₃ (403.98 mg, 0.441 mmol, 0.1 equiv). After stirring for 16 h at 90° C. under a nitrogen atmosphere. The reaction progress was monitored by LCMS. Desired product could be detected by LCMS. The reaction was quenched with water. The resulting mixture was extracted with EtOAc (2×50 ml). The combined organic layers were dried over anhydrous Na₂SO₄. After filtration, the filtrate was concentrated under reduced pressure. The residue was purified by silica gel column chromatography, eluted with petroleum ether/EtOAc (1/1) to afford 122c (1.6 g, 68.22%) as a yellow solid. LCMS (ESI, m/z): [M+H]⁺ 532.2

### Step C. 1-{3-[4-(dimethoxymethyl)piperidin-1-yl]benzenesulfonyl}piperidin-4-amine (122d)

To a solution of benzyl 112c (500 mg, 0.940 mmol, 1 equiv) in THF (10 mL) was added Pd/C (50 mg, 0.470 mmol, 0.50 equiv) under nitrogen atmosphere in a 50 ml round-bottom flask. The mixture was hydrogenated at room temperature for 16 h under hydrogen atmosphere using a hydrogen balloon. The reaction progress was monitored by LCMS. Desired product could be detected by LCMS. The mixture was filtered through a Celite pad and concentrated under reduced pressure. The crude product was used in the next step directly without further purification. This resulted in 122d (350 mg, 93.62%) as a black solid. LCMS (ESI, m/z): [M+H]⁺ 398.2

### Step D. 2′-[(1-{3-[4-(dimethoxymethyl)piperidin-1-yl]benzenesulfonyl}piperidin-4-yl)amino]-7′-[(1R,3R)-3-(oxan-2-yloxy)cyclohexyl]spiro[cyclopropane-1,5′-pyrrolo[2,3-d]pyrimidin]-6′-one (122e)

To a solution of 122d (300 mg, 0.755 mmol, 1 equiv) and Int-5 (342.20 mg, 0.906 mmol, 1.2 equiv) in 1,4-dioxane (6 mL) were added Cs₂CO₃ (737.65 mg, 2.265 mmol, 3 equiv) and RuPhos Pd G3 (63.12 mg, 0.076 mmol, 0.1 equiv) under nitrogen atmosphere. After stirring for 4 h at 90° C. under a nitrogen atmosphere. The reaction progress was monitored by LCMS. Desired product could be detected by LCMS. The resulting mixture was extracted with EA (2×10 ml) and dried over anhydrous Na₂SO₄. After filtration, the filtrate was concentrated under reduced pressure. The residue was purified by Prep-TLC (DCM/EtOH 15/1) to afford 122e (200 mg, 35.86%) as a yellow solid. LCMS (ESI, m/z): [M+H]⁺ 739.4

### Step E. 1-[3-(4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl)phenyl]piperidine-4-carbaldehyde (122f)

To a stirred solution of 122e (150 mg, 0.203 mmol, 1 equiv) in anhydrous DCM (7.5 mL) was added TFA (1.5 mL) at 0° C. and stirred for 1 h. The reaction progress was monitored by LCMS. Desired product could be detected by LCMS. The reaction was quenched by the addition of Na₂HCO₃ (10 ml) at 0° C. The aqueous layer was extracted with DCM (2×10 ml). The crude product was used in the next step directly without further purification. This resulted in 122f (150 mg, crude) as a yellow oil. LCMS (ESI, m/z): [M+H]⁺ 609.3

### Step F. 3-{5-[4-({1-[3-(4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl)phenyl]piperidin-4-yl}methyl)piperazin-1-yl]-1-oxo-3H-isoindol-2-yl}piperidine-2,6-dione (122)

To a stirred solution of 122f (100 mg, 0.164 mmol, 1 equiv), Int-29 (64.73 mg, 0.197 mmol, 1.2 equiv), HOAc (2.96 mg, 0.049 mmol, 0.3 equiv) in anhydrous DMF (3 mL). The reaction mixture was stirred at 50° C. for a period of 1 h. Then NaBH₃CN (30.97 mg, 0.492 mmol, 3 equiv) was added dropwise and the mixture was stirred for another 1 h. Desired product could be detected by LCMS. Desired product could be detected by LCMS. The residue was purified by reversed-phase flash chromatography with the following conditions: column, C18 silica gel; mobile phase, 0.1% FA in ACN, 10% to 50% gradient in 10 min; detector, UV 254 nm. This resulted in 3-{5-[4-({1-[3-(4-{7′-[(1R,3R)-3-hydroxycyclohexyl]-6′-oxospiro [cyclopropane-1,5′-pyrrolo[2,3-d]pyrimidin]-2′-ylamino}piperidin-1-ylsulfonyl)phenyl]piperidin-4-yl}methyl)piperazin-1-yl]-1-oxo-3H-isoindol-2-yl}piperidine-2,6-dione (122, 16.5 mg, 10.73%) as a white solid. LCMS (ESI, m/z): [M+H]⁺ 921.5

### Example 124. 3-{5-[4-({4-[3-(4-{7′-[(1R,3R)-3-hydroxycyclohexyl]-6′-oxospiro [cyclopropane-1,5′-pyrrolo[2,3-d]pyrimidin]-2′-ylamino}benzenesulfonyl)phenyl]piperazin-1-yl}methyl)piperidin-1-yl]-1-oxo-3H-isoindol-2-yl}piperidine-2,6-dione (124)

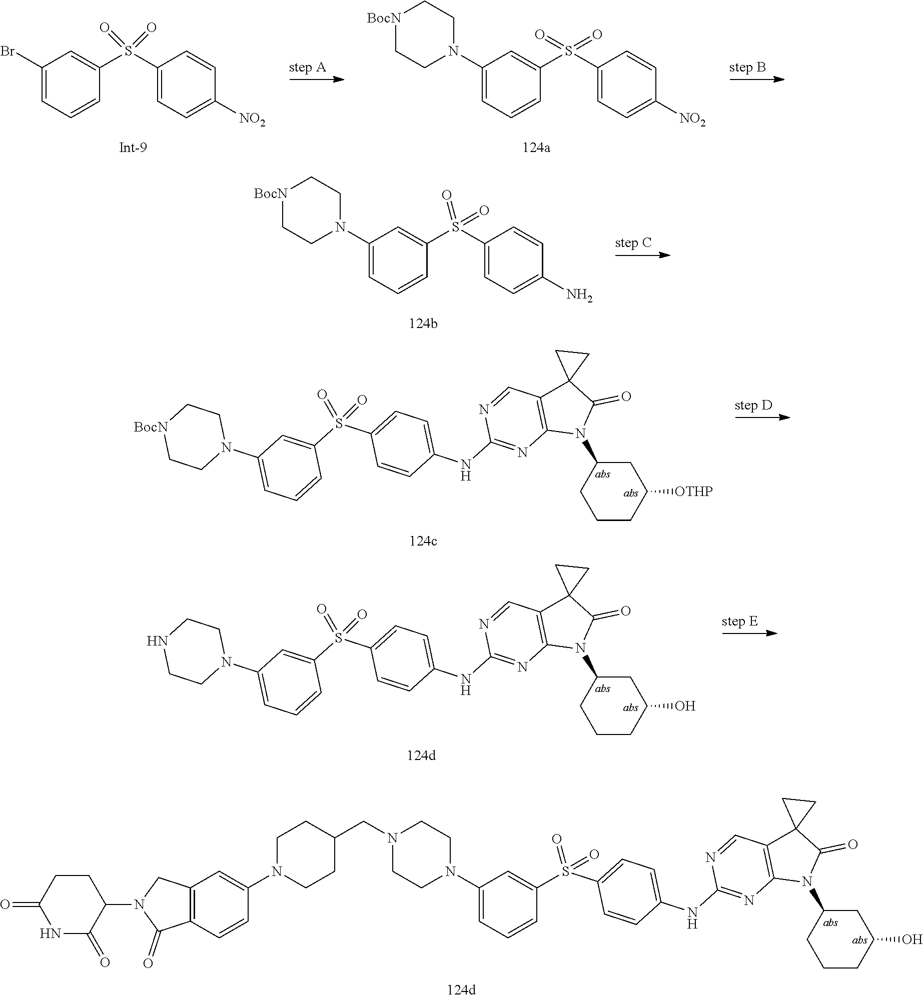

### Step A. tert-butyl 4-(3-((4-nitrophenyl)sulfonyl)phenyl)piperazine-1-carboxylate (124a)

A solution of Int-9 (500 mg, 1.461 mmol, 1 equiv) and tert-butyl piperazine-1-carboxylate (544.35 mg, 2.922 mmol, 2 equiv), CuI (55.66 mg, 0.292 mmol, 0.2 equiv), L-proline (33.65 mg, 0.292 mmol, 0.2 equiv) and K₂CO₃ (706.86 mg, 5.114 mmol, 3.5 equiv) in DMSO (5 mL) was stirred for overnight at 90° C. under argon atmosphere. Desired product could be detected by LCMS. The resulting mixture was diluted with ethyl acetate (50 mL). The resulting mixture was washed with H₂O (3×10 mL), dried over anhydrous Na₂SO₄. After filtration, the filtrate was concentrated under reduced pressure. The residue was purified by silica gel column chromatography, eluted with EtOAc/petroleum ether (1/3) to afford 124a (220 mg, 33.64%) as a white solid. LCMS (ESI, m/z): [M+H]⁺ 448.5.

### Step B. tert-butyl 4-(3-((4-aminophenyl)sulfonyl)phenyl)piperazine-1-carboxylate (124b)

To a solution of tert-butyl 124a (400 mg, 0.894 mmol, 1 equiv) in 15 mL CF₃CH₂OH was added Pd/C (10%, 200 mg) under nitrogen atmosphere in a 50 mL round-bottom flask. The mixture was hydrogenated at room temperature for 5 h under hydrogen atmosphere using a hydrogen balloon, filtered through a Celite pad and concentrated under reduced pressure. Desired product could be detected by LCMS. The resulting mixture was filtered, the filter cake was washed with MeOH (3×10 mL). The filtrate was concentrated under reduced pressure. The resulting mixture was concentrated under reduced pressure to afford 124b (350 mg, 81.46%) as a light yellow solid. LCMS (ESI, m/z): [M+H]⁺ 418.1.

### Step C. tert-butyl 4-(3-((4-((6′-oxo-7′-((1R,3R)-3-((tetrahydro-2H-pyran-2-yl)oxy)cyclohexyl)-6′,7′-dihydrospiro[cyclopropane-1,5′-pyrrolo[2,3-d]pyrimidin]-2′-yl)amino)phenyl)sulfonyl)phenyl) piperazine-1-carboxylate (124c)

To a stirred solution of tert-butyl 124b (350 mg, 0.838 mmol, 1 equiv) and Int-5 (380.11 mg, 1.006 mmol, 1.2 equiv) in dioxane (4 mL) were added RuPhos Pd G₃ (105.25 mg, 0.126 mmol, 0.15 equiv) and Cs₂CO₃ (819.39 mg, 2.514 mmol, 3 equiv) in portions at room temperature under nitrogen atmosphere. The resulting mixture was stirred for 1 h at 90° C. under nitrogen atmosphere. Desired product could be detected by LCMS. The resulting mixture was extracted with EtOAc (3×10 mL). The combined organic layers were washed with brine (3×5 mL), dried over anhydrous Na₂SO₄. The resulting mixture was concentrated under reduced pressure. The residue was purified by Prep-TLC (petroleum ether/EtOAc=1:1) to afford 124c (300 mg, 42.30%) as a yellow solid. LCMS (ESI, m/z): [M+H]759.3.

### Step D. 7′-[(1R,3R)-3-hydroxycyclohexyl]-2′-({4-[3-(piperazin-1-yl)benzenesulfonyl]phenyl}amino) spiro[cyclopropane-1,5′-pyrrolo[2,3-d]pyrimidin]-6′-one (124d)

To a stirred mixture of tert-butyl 124c (200 mg, 0.264 mmol, 1 equiv) in MeCN (5 mL) was added HCl (gas) in 1,4-dioxane (2 mL) dropwise at 0° C. under nitrogen atmosphere. The resulting mixture was stirred for 1 h at 0° C. under Hydrogen chloride atmosphere. Desired product could be detected by LCMS. The resulting mixture was concentrated under reduced pressure. The residue was purified by reversed-phase flash chromatography with the following conditions: column, C18 silica gel; mobile phase, MeCN in Water, 10% to 30% gradient in 30 min; detector, UV 254 nm. The resulting mixture was concentrated under reduced pressure to afford 124d (100 mg, 66.03%) as a light yellow solid. LCMS (ESI, m/z): [M+H]⁺ 575.2.

### Step E. 3-{5-[4-({4-[3-(4-{7′-[(1R,3R)-3-hydroxycyclohexyl]-6′-oxospiro[cyclopropane-1,5′-pyrrolo[2,3-d]pyrimidin]-2′-ylamino}benzenesulfonyl)phenyl]piperazin-1-yl}methyl)piperidin-1-yl]-1-oxo-3H-isoindol-2-yl}piperidine-2,6-dione (124)

A solution of 124d (80 mg, 0.139 mmol, 1 equiv) in DMF (2 mL) was treated with Int-28 (54.42 mg, 0.153 mmol, 1.1 equiv) and HOAc (2.51 mg, 0.042 mmol, 0.3 equiv) for 1 h at 50° C. under nitrogen atmosphere followed by the addition of NaBH₃CN (17.50 mg, 0.278 mmol, 2 equiv) in portions at room temperature. The resulting mixture was stirred for 2 h at room temperature under nitrogen atmosphere. Desired product could be detected by LCMS. The residue was purified by reversed-phase flash chromatography with the following conditions: column, C18 silica gel; mobile phase, MeCN in Water (10 mmol/L NH₄HCO₃), 5% to 50% gradient in 35 min. The resulting mixture was concentrated under reduced pressure. This resulted in 3-{5-[4-({4-[3-(4-{7′-[(1R,3R)-3-hydroxycyclohexyl]-6′-oxospiro[cyclopropane-1,5′-pyrrolo [2,3-d]pyrimidin]-2′-ylamino}benzenesulfonyl) phenyl]piperazin-1-yl}methyl)piperidin-1-yl]-1-oxo-3H-isoindol-2-yl}piperidine-2,6-dione (124, 52.4 mg, 40.40%) as a white solid. LCMS (ESI, m/z): [M+H]⁺ 914.5

### Example 125. 3-[5-({3-[3-(4-{7′-[(1R,3R)-3-hydroxycyclohexyl]-6′-oxospiro[cyclopropane-1,5′-pyrrolo [2,3-d]pyrimidin]-2′-ylamino}benzenesulfonyl)phenoxy]azetidin-1-yl}methyl)-1-oxo-3H-isoindol-2-yl]piperidine-2,6-dione (125)

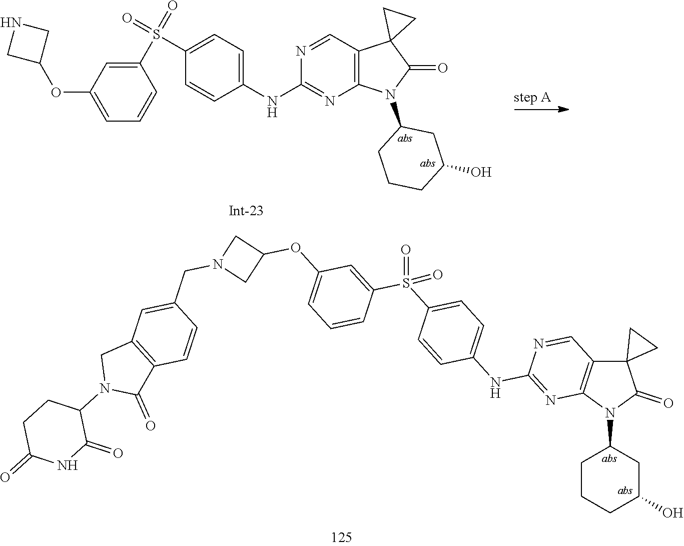 [SMILES: O=C1N([C@@H]2CCC[C@@H](O)C2)c2nc(Nc3ccc(S(=O)(=O)c4cccc(OC5CNC5)c4)cc3)ncc2C12CC2 ; O=C1CCC(N2Cc3cc(CN4CC(Oc5cccc(S(=O)(=O)c6ccc(Nc7ncc8c(n7)N([C@@H]7CCC[C@@H](O)C7)C(=O)C87CC7)cc6)c5)C4)ccc3C2=O)C(=O)N1]

### Step A. 3-[5-({3-[3-(4-{7′-[(1R,3R)-3-hydroxycyclohexyl]-6′-oxospiro[cyclopropane-1,5′-pyrrolo[2,3-d]pyrimidin]-2′-ylamino}benzenesulfonyl)phenoxy]azetidin-1-yl}methyl)-1-oxo-3H-isoindol-2-yl]piperidine-2,6-dione (125)

To a stirred solution of Int-23 (150 mg, 0.267 mmol, 1 equiv) and Int-31 (72.71 mg, 0.267 mmol, 1 equiv) in DMF (2 mL) was added HOAc (4.81 mg, 0.080 mmol, 0.3 equiv) in portions at room temperature under air atmosphere. The resulting mixture was stirred for 1 h at 50° C. under air atmosphere. To the above mixture was added NaBH₃CN (50.35 mg, 0.801 mmol, 3 equiv). The resulting mixture was stirred for additional 1 h at room temperature. Desired product could be detected by LCMS. The residue was purified by reversed-phase flash chromatography with the following conditions: column, C18 silica gel; mobile phase, MeCN in Water (10 mmol/L NH₄HCO₃), 10% to 50% gradient in 30 min to afford 3-[5-({3-[3-(4-{7′-[(1R,3R)-3-hydroxycyclohexyl]-6′-oxospiro[cyclopropane-1,5′-pyrrolo[2,3-d]pyrimidin]-2′-ylamino}benzenesulfonyl)phenoxy]azetidin-1-yl}methyl)-1-oxo-3H-isoindol-2-yl]piperidine-2,6-dione (125, 41.7 mg, 18.73%) as a white solid. LCMS (ESI, m/z): [M+H]⁺ 818.3

### Example 126. 3-{5-[4-({1-[3-(4-{7′-[(1R,3R)-3-hydroxycyclohexyl]-6′-oxospiro [cyclopropane-1,5′-pyrrolo[2,3-d]pyrimidin]-2′-ylamino}benzenesulfonyl) phenyl]piperidin-4-yl}methyl)piperazin-1-yl]-1-oxo-3H-isoindol-2-yl}piperidine-2,6-dione (126)

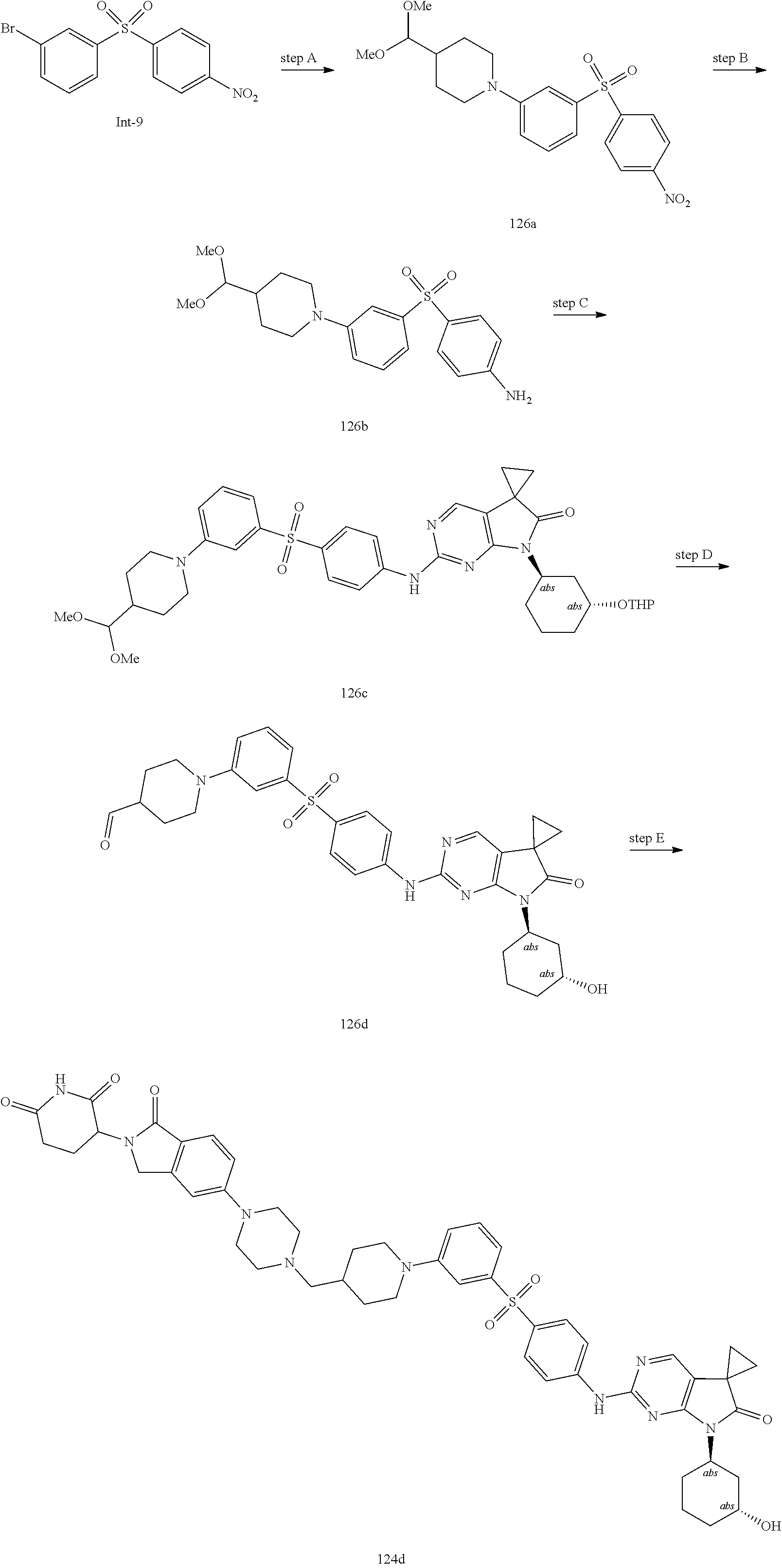

### Step A. 4-(Dimethoxymethyl)-1-[3-(4-nitrobenzenesulfonyl)phenyl]piperidine (126a)

A solution of Int-9 (500 mg, 1.461 mmol, 1 equiv) and tert-butyl 4-(dimethoxymethyl)piperazine-1-carboxylate (760.85 mg, 2.922 mmol, 2 equiv), CuI (55.66 mg, 0.292 mmol, 0.2 equiv), L-proline (33.65 mg, 0.292 mmol, 0.2 equiv) and K₂CO₃ (706.86 mg, 5.114 mmol, 3.5 equiv) in DMSO (5 mL) was stirred for overnight at 90° C. under argon atmosphere. Desired product could be detected by LCMS. The resulting mixture was diluted with ethyl acetate (50 mL). The resulting mixture was washed with H₂O (3×10 mL), dried over anhydrous Na₂SO₄. After filtration, the filtrate was concentrated under reduced pressure. The residue was purified by silica gel column chromatography, eluted with petroleum ether/EtOAc (3/1) to afford 126a (160 mg, 26.04%) as a white solid. LCMS (ESI, m/z): [M+H]⁺ 421.1

### Step B. 4-{3-[4-(Dimethoxymethyl)piperidin-1-yl]benzenesulfonyl}aniline (126b)

To a solution of 126a (660 mg, 1.570 mmol, 1 equiv) in CF₃CH₂OH (10 mL) was added Pd/C (130 mg, 0.122 mmol, 0.08 equiv, 10%) under nitrogen atmosphere. The mixture was hydrogenated at room temperature for 2 h under hydrogen atmosphere using a hydrogen balloon. Desired product could be detected by LCMS. The resulting mixture was filtered, the filter cake was washed with DCM. The filtrate was concentrated under reduced pressure. The residue was purified by Prep-TLC (petroleum ether/EtOAc=1:1) to afford 126b (500 mg, 81.57%) as a yellow solid. LCMS (ESI, m/z): [M+H]⁺ 391.1

### Step C. 2′-[(4-{3-[4-(dimethoxymethyl)piperidin-1-yl]benzenesulfonyl}phenyl)amino]-7′-[(1R,3R)-3-(oxan-2-yloxy)cyclohexyl]spiro[cyclopropane-1,5′-pyrrolo[2,3-d]pyrimidin]-6′-one (126c)

To a solution of 126b (300 mg, 0.794 mmol, 1 equiv) and Int-5 (465.04 mg, 1.191 mmol, 1.5 equiv) in dioxane (4 mL) were added BINAP (98.87 mg, 0.159 mmol, 0.2 equiv), Cs₂CO₃ (776.03 mg, 2.382 mmol, 3 equiv) and RuPhos Pd G3 (99.60 mg, 0.119 mmol, 0.15 equiv). After stirring for 1 h at 90° C. under a nitrogen atmosphere, the resulting mixture was concentrated under reduced pressure. Desired product could be detected by LCMS. The residue was purified by silica gel column chromatography, eluted with CH₂Cl₂/MeOH (10:1) to afford 126c (250 mg, 30.16%) as a yellow solid. LCMS (ESI, m/z): [M+H]⁺ 732.3

### Step D. 1-[3-(4-{7′-[(1R,3R)-3-hydroxycyclohexyl]-6′-oxospiro[cyclopropane-1,5′-pyrrolo[2,3-d]pyrimidin]-2′-ylamino}benzenesulfonyl)phenyl]piperidine-4-carbaldehyde (126d)

To a stirred solution of 126c (200 mg, 0.273 mmol, 1 equiv) in DCM (2 mL) was added TFA (2 mL) dropwise at 0° C. The resulting mixture was stirred for 40 min at 0° C. Desired product could be detected by LCMS. The mixture was basified to pH 7 with saturated NaHCO₃ (aq.). The resulting mixture was concentrated under reduced pressure to afford the product of 126d (200 mg, crude) as a white solid. The crude product was used in the next step directly without further purification. LCMS (ESI, m/z): [M+H]⁺ 602.2

### Step E. 3-{5-[4-({1-[3-(4-{7′-[(1R,3R)-3-hydroxycyclohexyl]-6′-oxospiro [cyclopropane-1,5′-pyrrolo[2,3-d]pyrimidin]-2′-ylamino}benzenesulfonyl)phenyl]piperidin-4-yl}methyl)piperazin-1-yl]-1-oxo-3H-isoindol-2-yl}piperidine-2,6-dione (126)

To a stirred solution of 126d (200 mg, 0.332 mmol, 1 equiv) and Int-29 (130.97 mg, 0.398 mmol, 1.2 equiv) in DMF (5 mL) were added HOAc (2.00 mg, 0.033 mmol, 0.1 equiv) and NaBH₃CN (62.66 mg, 0.996 mmol, 3 equiv) in portions at room temperature. The resulting mixture was stirred for 1 h at 30° C. Desired product could be detected by LCMS. The aqueous layer was extracted with EtOAc (3×50 mL). The resulting mixture was concentrated under reduced pressure. The crude product (100 mg) was purified by Prep-HPLC with the following conditions (Column: Xbridge Phenyl OBD Column, 19*150 mm, 5 m; Mobile Phase A: Water (10 mmol/L NH4HCO3, Mobile Phase B: ACN; Flow rate: 60 mL/min mL/min; Gradient: 35% B to 50% B in 10 min; Wave Length: 254 nm/220 nm nm; RT1 (min): 10.58) to afford 3-{5-[4-({1-[3-(4-{7′-[(1R,3R)-3-hydroxycyclohexyl]-6′-oxospiro[cyclopropane-1,5′-pyrrolo[2,3-d]pyrimidin]-2′-ylamino}benzenesulfonyl) phenyl]piperidin-4-yl}methyl)piperazin-1-yl]-1-oxo-3H-isoindol-2-yl}piperidine-2,6-dione (126, 33.6 mg, 10.61%) as a white solid. LCMS (ESI, m/z): [M+H]⁺ 914.4

### Example 130: 3-(4-(4-((4-((4-((7′-((1R,3R)-3-Hydroxycyclohexyl)-6′-oxo-6′,7′-dihydrospiro[cyclopropane-1,5′-pyrrolo[2,3-d]pyrimidin]-2′-yl)amino)piperidin-1-yl)sulfonyl)-1H-pyrazol-1-yl)methyl)piperidin-1-yl)-3-methyl-2-oxo-2,3-dihydro-1H-benzo[d]imidazol-1-yl)piperidine-2,6-dione (130)

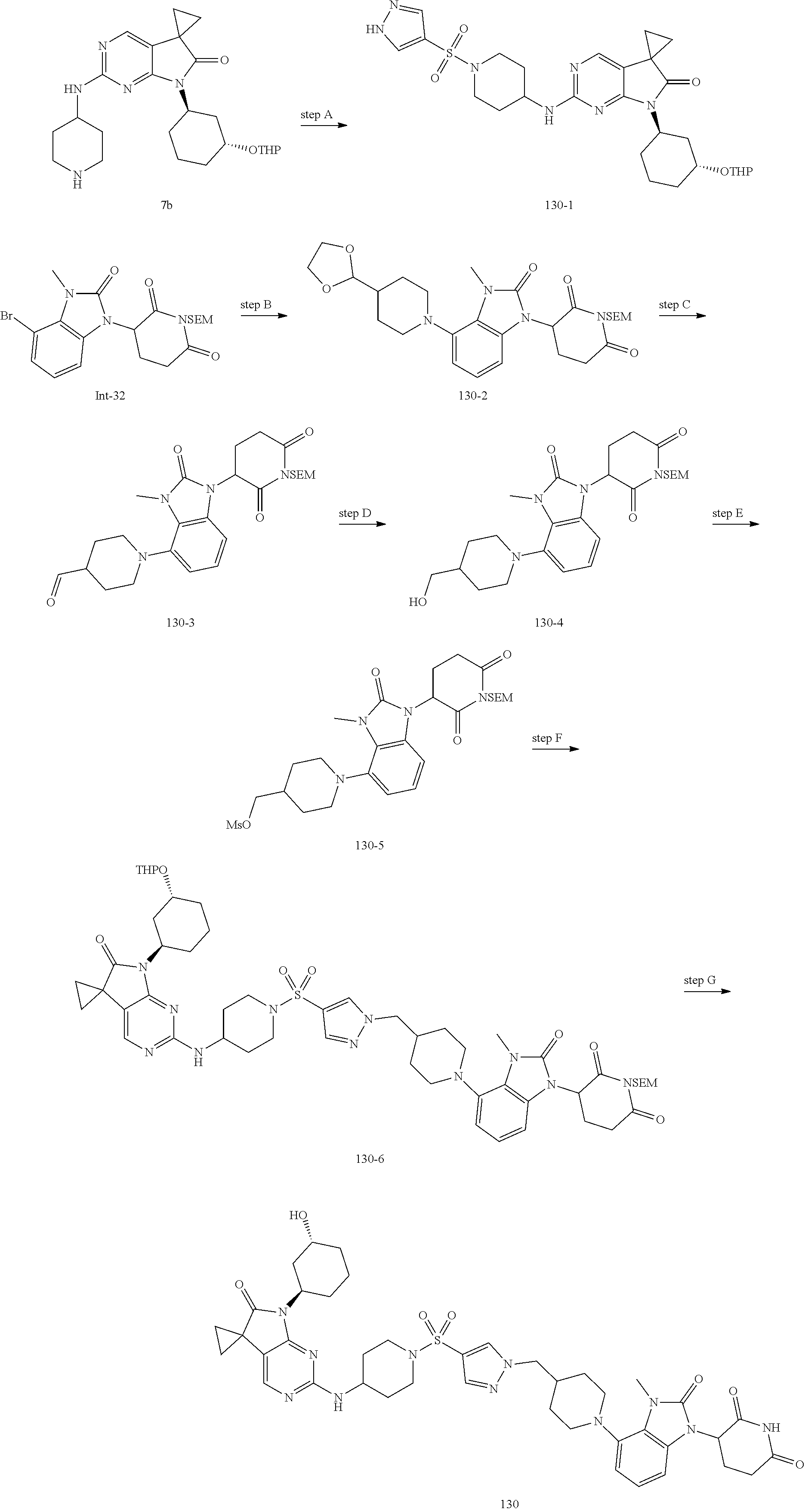

### Step A. 7′-[(1S,3S)-3-(Oxan-2-yloxy)cyclohexyl]-2′-{[1-(1H-pyrazole-4-sulfonyl)piperidin-4-yl]amino}spiro[cyclopropane-1,5′-pyrrolo[2,3-d]pyrimidin]-6′-one (130-1)

To a solution of 7b (100 mg, 0.226 mmol, 1 equiv) in DCM (5 mL) was added TEA (68.75 mg, 0.678 mmol, 3 equiv) and 1H-pyrazole-4-sulfonyl chloride (37.72 mg, 0.226 mmol, 1 equiv) at 0 degrees C. The resulting mixture was stirred for 1 h at 25 degrees C. The resulting mixture was diluted with water (5 mL) and extracted with EtOAc (3×10 mL). The combined organic layers were dried over anhydrous Na₂SO₄ and concentrated under reduced pressure. The residue was purified by silica gel column chromatography, eluted with DCM/THF (1:5) to afford 130-1 (80 mg, 46.34%) as a yellow solid. LCMS (ESI, m/z): [M+H]⁺=572.

### Step B. 3-{4-[4-(1,3-Dioxolan-2-yl)piperidin-1-yl]-3-methyl-2-oxo-1,3-benzodiazol-1-yl}-1-{[2-(trimethylsilyl)ethoxy]methyl}piperidine-2,6-dione (130-2)

To a mixture of Int-32 (400 mg, 0.854 mmol, 1 equiv) and 4-(1,3-dioxolan-2-yl)piperidine (134.25 mg, 0.854 mmol, 1 equiv) and Pd-PEPPSI-IPentCl 2-methylpyridine (o-picoline) (71.83 mg, 0.085 mmol, 0.1 equiv) in dioxane (10 mL) were added K₂CO₃ (354.05 mg, 2.562 mmol, 3 equiv) at 25 degrees C. under N2. The resulting mixture was stirred for 12 h at 110 degrees C. The mixture was concentrated under vacuum. The resulting mixture was diluted with water (50 mL) and extracted with EtOAc (3×100 mL). The combined organic layers were dried over anhydrous Na₂SO₄ and concentrated under reduced pressure. The residue was purified by silica gel column chromatography, eluted with PE/EA (1:1) to afford 130-2 (210 mg, 33.86%) as a yellow solid. LCMS (ESI, m/z): [M+H]⁺=545.

### Step C. 1-[1-(2,6-dioxo-1-{[2-(trimethylsilyl) ethoxy]methyl}piperidin-3-yl)-3-methyl-2-oxo-1,3-benzodiazol-4-yl]piperidine-4-carbaldehyde (130-3)

To a stirred solution of 130-2 (100 mg, 0.184 mmol, 1 equiv) in Acetone (5 mL) and H₂O (1 mL) was added TsOH (126.45 mg, 0.736 mmol, 4 equiv) at 25 degrees C. under N₂. The resulting mixture was stirred for 1 h at 60 degrees C. The resulting mixture was diluted with water (50 mL) and extracted with EtOAc (3×100 mL). The combined organic layers were dried over anhydrous Na₂SO₄ and concentrated under reduced pressure. The residue was purified by silica gel column chromatography, eluted with PE/EA (1:1) to afford 130-3 (60 mg, 48.96%) as a yellow solid. LCMS (ESI, m/z): [M+H]⁺=501.

### Step D. 3-{4-[4-(Hydroxymethyl)piperidin-1-yl]-3-methyl-2-oxo-1,3-benzodiazol-1-yl}-1-{[2-(trimethylsilyl)ethoxy]methyl}piperidine-2,6-dione (130-4)

To a solution of 130-3 (100 mg, 0.200 mmol, 1 equiv) in DMF (5 mL) was added STAB (126.99 mg, 0.600 mmol, 3 equiv) at 25 degrees C. under N₂. The resulting mixture was stirred for 1 h at 60 degrees C. The resulting mixture was diluted with water (50 mL) and extracted with EtOAc (3×100 mL). The combined organic layers were dried over anhydrous Na₂SO₄ and concentrated under reduced pressure. The residue was purified by reversed-phase Combi-Flash to afford 130-4 (50 mg, 42.33%) as a yellow solid. LCMS (ESI, m/z): [M+H]⁺=503.

### Step E. {1-[1-(2,6-Dioxo-1-{[2-(trimethylsilyl)ethoxy]methyl}piperidin-3-yl)-3-methyl-2-oxo-1,3-benzodiazol-4-yl]piperidin-4-yl}methyl methanesulfonate (130-5)

To a stirred mixture of 130-4 (200 mg, 0.398 mmol, 1 equiv) and TEA (161.04 mg, 1.592 mmol, 4 equiv) in DCM (10 mL) were added methanesulfonic anhydride (138.61 mg, 0.796 mmol, 2 equiv) at 0 degrees C. The resulting mixture was stirred for 1 h at 25 degrees C. The resulting mixture was diluted with water (5 mL) and extracted with EtOAc (3×10 mL). The combined organic layers were dried over anhydrous Na₂SO₄ and concentrated under reduced pressure. The residue was purified by silica gel column chromatography, eluted with PE/EA (1:2) to afford 130-5 (180 mg, 58.42%) as a yellow solid. LCMS (ESI, m/z): [M+H]⁺=581.

### Step F. 3-[3-Methyl-4-(4-{[4-(4-{7′-[(1S,3S)-3-(oxan-2-yloxy)cyclohexyl]-6′-oxospiro[cyclopropane-1,5′-pyrrolo[2,3-d]pyrimidin]-2′-ylamino}piperidin-1-ylsulfonyl)pyrazol-1-yl]methyl}piperidin-1-yl)-2-oxo-1,3-benzodiazol-1-yl]-1-{[2-(trimethylsilyl)ethoxy]methyl}piperidine-2,6-dione (130-6)

To a stirred mixture of 130-1 (110 mg, 0.192 mmol, 1 equiv) and 130-5 (111.75 mg, 0.192 mmol, 1 equiv) in MeCN (5 mL) were added K₂CO₃ (79.78 mg, 0.576 mmol, 3 equiv) at 25 degrees C. The resulting mixture was stirred for 12 h at 60° C. The resulting mixture was diluted with water (5 mL) and extracted with EtOAc (3×10 mL). The combined organic layers were dried over anhydrous Na₂SO₄ and concentrated under reduced pressure. The residue was purified by silica gel column chromatography, eluted with DCM/THF (1:5) to afford 130-6 (60 mg, 25.39%) as a yellow solid. LCMS (ESI, m/z): [M+H]⁺=1056.

### Step G. 3-[5-(4-{1-[3-(4-{7′-[(1S,3S)-3-hydroxycyclohexyl]-6′-oxospiro[cyclopropane-1,5′-pyrrolo[2,3-d]pyrimidin]-2′-ylamino}piperidin-1-ylsulfonyl)phenyl]azetidin-3-yl}piperazin-1-yl)-1-oxo-3H-isoindol-2-yl]piperidine-2,6-dione (130)

To a stirred solution of 130-6 (80 mg, 0.083 mmol, 1 equiv) in DCM (3 mL) was added 4M HCl (gas) in 1,4-dioxane (0.6 mL) at 0 degrees C. The resulting mixture was stirred for 1 h at 25 degrees C. The mixture was concentrated under vacuum. The resulting mixture was purified by reversed-phase to afford compound 130 (19.2 mg, 25.22%) as a white solid. LCMS (ESI, m/z): [M+H]⁺=842.4.

### Example 131: N-(3-{4-[1-(2,6-Dioxopiperidin-3-yl)-3-methyl-2-oxo-1,3-benzodiazol-4-yl]piperidin-1-yl}propyl)-4-17′-[(1R,3R)-3-hydroxycyclohexyl]-6′-oxospiro[cyclopropane-1,5′-pyrrolo[2,3-d]pyrimidin]-2′-ylamino}-N-methylpiperidine-1-sulfonamide (131)

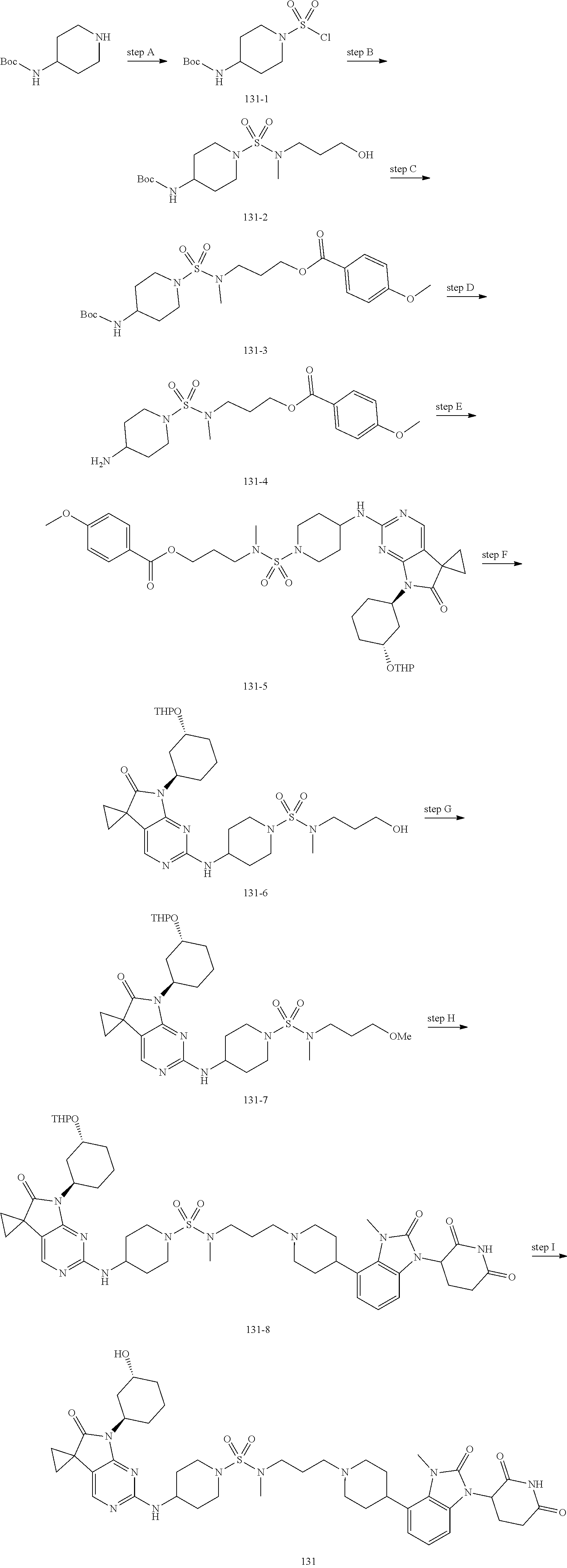

### Step A. Tert-butyl N-[1-(chlorosulfonyl)piperidin-4-yl]carbamate (131-1)

To a stirred solution of tert-butyl N-(piperidin-4-yl)carbamate (1 g, 4.993 mmol, 1 equiv) and TEA (0.76 g, 7.490 mmol, 1.5 equiv) in DCM (20 mL) was added SO₂Cl₂ (0.81 g, 5.992 mmol, 1.2 equiv) dropwise at 0° C. The resulting mixture was stirred for overnight at room temperature. The resulting mixture was diluted with water. The resulting mixture was extracted with CH₂Cl₂. The combined organic layers were dried over anhydrous Na₂SO₄. After filtration, the filtrate was concentrated under reduced pressure to afford 131-1 (1.2 g, 80.44%) as a white solid.

### Step B. Tert-butyl N-{1-[(3-hydroxypropyl)(methyl)sulfamoyl]piperidin-4-yl}carbamate (131-2)

A solution of 131-1 (2 g, 6.694 mmol, 1 equiv) and 3-(methylamino)propan-1-ol (0.90 g, 10.041 mmol, 1.5 equiv), TEA (2.03 g, 20.082 mmol, 3 equiv) in DCM (10 mL) was stirred for 1 h at room temperature. Desired product could be detected by LCMS. The resulting mixture was concentrated under reduced pressure. The residue was purified by silica gel column chromatography, eluted with CH₂Cl₂/MeOH (20:1) to afford 131-2 (1.5 g, 63.76%) as a white solid. LCMS (ESI, m/z): [M+H]⁺=352.

### Step C. 3-((4-((tert-butoxycarbonyl)amino)-N-methylpiperidine)-1-sulfonamido)propyl 4-methoxybenzoate (131-3)

A solution of 131-2 (600 mg, 1.707 mmol, 1 equiv) and P-anisic acid (259.74 mg, 1.707 mmol, 1 equiv) and DMAP (166.85 mg, 1.366 mmol, 0.8 equiv) and DIC (236.99 mg, 1.878 mmol, 1.1 equiv) in DCM (10 mL) was stirred for 15 h at room temperature under nitrogen atmosphere. The resulting mixture was concentrated under reduced pressure. The residue was purified by silica gel column chromatography, eluted with PE/EA (6:1) to afford 131-3 (800 mg, 90.4%) as a white solid. LCMS (ESI, m/z): [M+H]⁺=486.

### Step D. 3-[(4-aminopiperidin-1-ylsulfonyl)(methyl)amino]propyl 4-methoxybenzoate (131-4)

A solution of 131-3 (800 mg, 1.647 mmol, 1 equiv) and 4M HCl in 1,4-dioxane (8 mL) in MeCN (30 mL) was stirred for 1 h at room temperature under nitrogen atmosphere. Desired product could be detected by LCMS. The resulting mixture was concentrated under reduced pressure to afford 131-4 (700 mg, crude) as a white solid. LCMS (ESI, m/z): [M+H]⁺=386.

### Step E. 3-[Methyl(4-{7′-[(1R,3R)-3-(oxan-2-yloxy)cyclohexyl]-6′-oxospiro[cyclopropane-1,5′-pyrrolo[2,3-d]pyrimidin]-2′-ylamino}piperidin-1-ylsulfonyl)amino]propyl 4-methoxybenzoate (131-5)

A solution of 131-4 (600 mg, 1.557 mmol, 1 equiv), Int-5 (470.52 mg, 1.246 mmol, 0.8 equiv) and BINAP (145.38 mg, 0.234 mmol, 0.15 equiv), RuPhos Pd G3 (130.17 mg, 0.156 mmol, 0.1 equiv) and Cs₂CO₃ (2028.55 mg, 6.228 mmol, 4 equiv) in 1,4-dioxane (10 mL) was stirred for 4 h at 100° C. under nitrogen atmosphere. Desired product could be detected by LCMS. The resulting mixture was concentrated under reduced pressure. The residue was purified by silica gel column chromatography, eluted with PE/EA (1:2) to afford 131-5 (500 mg, 41.98%) as a yellow solid. LCMS (ESI, m/z): [M+H]⁺=727.

### Step F. 2′-({1-[(3-Hydroxypropyl) (methyl)sulfamoyl]piperidin-4-yl}amino)-7′-[(1R,3R)-3-(oxan-2-yloxy)cyclohexyl]spiro[cyclopropane-1,5′-pyrrolo[2,3-d]pyrimidin]-6′-one (131-6)

A solution of 131-5 (600 mg, 0.825 mmol, 1 equiv) and LiOH (118.61 mg, 4.950 mmol, 6 equiv) in THF (6 mL)/H₂O (2 mL) was stirred for 48 h at room temperature under nitrogen atmosphere. Desired product could be detected by TLC (PE:EA=1:1, Rf=0.4). The resulting mixture was concentrated under reduced pressure. The residue was purified by silica gel column chromatography, eluted with PE/EA (1:1) to afford 131-6 (400 mg, 77.66%) as a yellow solid. LCMS (ESI, m/z): [M+H]⁺=593.

### Step G. 3-[Methyl(4-{7′-[(1R,3R)-3-(oxan-2-yloxy)cyclohexyl]-6′-oxospiro[cyclopropane-1,5′-pyrrolo[2,3-d]pyrimidin]-2′-ylamino}piperidin-1-ylsulfonyl)amino]propyl methanesulfonate (131-7)

A solution of 131-6 (200 mg, 0.337 mmol, 1 equiv) and DIEA (87.22 mg, 0.674 mmol, 2 equiv) in DCM (5 mL) was added MsCl (38.65 mg, 0.337 mmol, 1 equiv) and stirred for 2 h at 0° C. under nitrogen atmosphere. Desired product could be detected by TLC (PE:THF=2:1). The resulting mixture was concentrated under reduced pressure. The residue was purified by Prep-TLC (PE/THF 2:1) to afford 131-7 (180 mg, 75.55%) as a light yellow solid. LCMS (ESI, m/z): [M+H]⁺=671.

### Step H. N-(3-{4-[1-(2,6-Dioxopiperidin-3-yl)-3-methyl-2-oxo-1,3-benzodiazol-4-yl]piperidin-1-yl}propyl)-N-methyl-4-{7′-[(1R,3R)-3-(oxan-2-yloxy)cyclohexyl]-6′-oxospiro[cyclopropane-1,5′-pyrrolo[2,3-d]pyrimidin]-2′-ylamino}piperidine-1-sulfonamide (131-8)

A solution of 131-7 (90 mg, 0.134 mmol, 1 equiv), 13-3 (45.94 mg, 0.134 mmol, 1 equiv) and K₂CO₃ (111.25 mg, 0.804 mmol, 6 equiv), KI (2.23 mg, 0.013 mmol, 0.1 equiv) in acetone (10 mL) was stirred for 15 h at 60° C. under nitrogen atmosphere. Desired product could be detected by TLC (THF:TEA=5:0.5, Rf=0.5). The residue was purified by Prep-TLC (THF:TEA=5:0.5, Rf=0.5) to afford 131-8 (80 mg, 61.77%) as a yellow solid. LCMS (ESI, m/z): [M+H]⁺=917.

### Step I. N-(3-{4-[1-(2,6-Dioxopiperidin-3-yl)-3-methyl-2-oxo-1,3-benzodiazol-4-yl]piperidin-1-yl}propyl)-4-{7′-[(1R,3R)-3-hydroxycyclohexyl]-6′-oxospiro[cyclopropane-1,5′-pyrrolo[2,3-d]pyrimidin]-2′-ylamino}-N-methylpiperidine-1-sulfonamide (131)

A solution of 131-8 (80 mg, 0.087 mmol, 1 equiv) and TFA (198.92 mg, 1.740 mmol, 20 equiv) in DCM (5 mL) was stirred for 1.5 h at 0° C. under nitrogen atmosphere. Desired product could be detected by LCMS. The reaction was quenched by the addition of TEA (1 mL) at 0° C. The resulting mixture was concentrated under reduced pressure. The crude product was purified by reverse phase flash to afford compound 131 (15.9 mg, 20.79%) as a white solid. LCMS (ESI, m/z): [M+H]⁺=833.4.

### Example 132: 3-[5-({3-[3-(4-{7′-[(1R,3R)-3-Hydroxycyclohexyl]-6′-oxospiro[cyclopropane-1,5′-pyrrolo[2,3-d]pyrimidin]-2′-ylamino}piperidin-1-ylsulfonyl)phenoxy]azetidin-1-yl}methyl)-1-oxo-3H-isoindol-2-yl]piperidine-2,6-dione (132)

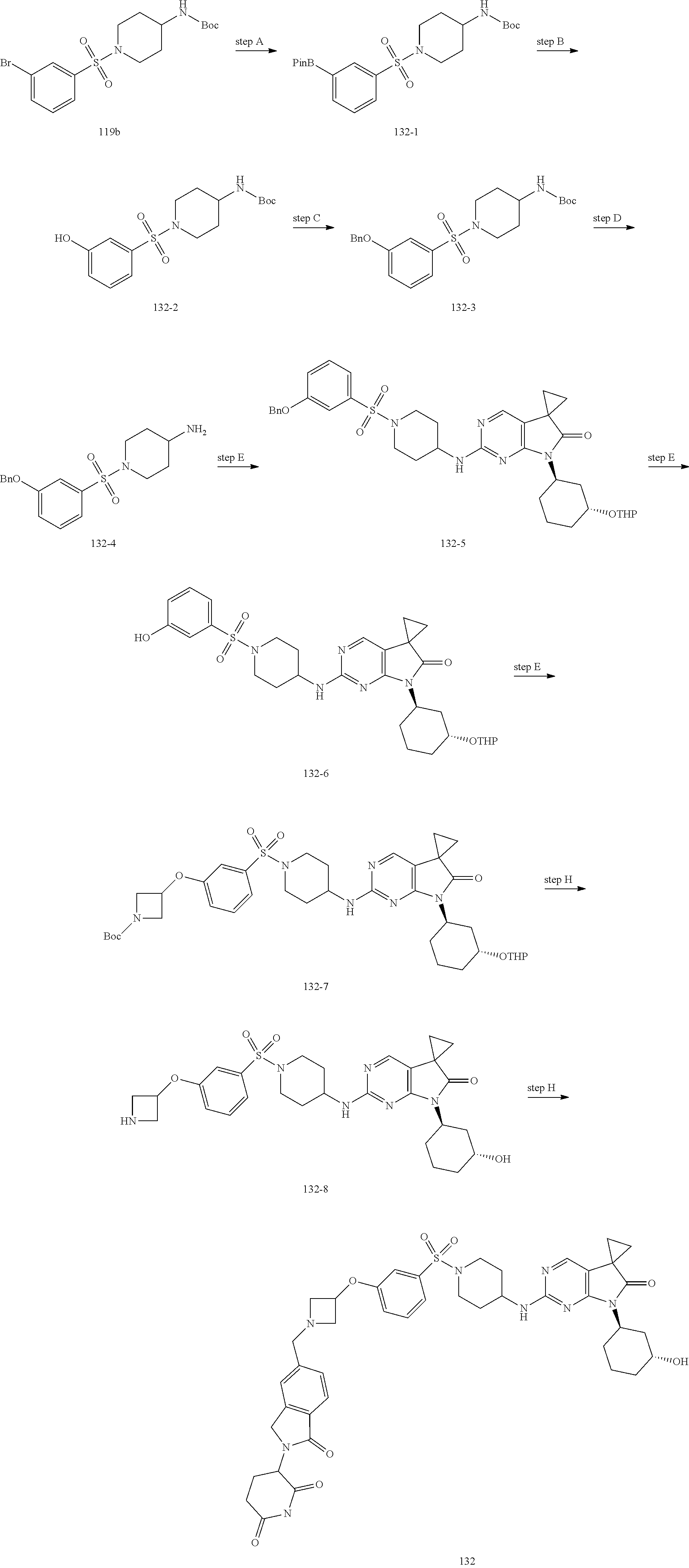

### Step A. Tert-butyl (1-((3-(4,4,5,5-tetramethyl-1,3,2-dioxaborolan-2-yl)phenyl) sulfonyl)piperidin-4-yl)carbamate (132-1)

To a mixture of 119b (16.1 g, 38.395 mmol, 1 equiv) and 4,4,4′,4′,5,5,5′,5′-octamethyl-2,2′-bi(1,3,2-dioxaborolane) (29.28 g, 115.185 mmol, 3 equiv) in dioxane (160 mL) was added Pd(dppf)Cl₂ (1.40 g, 1.920 mmol, 0.05 equiv) and KOAc (18.84 g, 191.975 mmol, 5 equiv) at room temperature. The resulting mixture was stirred for overnight at 90° C. under nitrogen atmosphere. The resulting mixture was diluted with water and extracted with EtOAc. The combined organic layers were dried over anhydrous Na₂SO₄ and concentrated under reduced pressure. The crude product mixture was used in the next step directly without further purification. LCMS (ESI, m/z): [M+H]⁺=467

### Step B. Tert-butyl N-[1-(3-hydroxybenzenesulfonyl)piperidin-4-yl]carbamate (132-2)

A solution of 132-1 (16.1 g, 34.520 mmol, 1 equiv) and 30% H₂O₂ (10 mL) was stirred for 2.5 h at room temperature. The reaction was quenched by the addition of sat. sodium hyposulfite (aq.) (15 mL) at room temperature. The resulting mixture was extracted with EtOAc (2×20 mL). The combined organic layers were washed with brine, dried over anhydrous Na₂SO₄. After filtration, the filtrate was concentrated under reduced pressure. The crude product was purified by Prep-HPLC to afford 132-2 (5.3 g, 43.07%) as a yellow solid. LCMS (ESI, m/z): [M+H]⁺=357.

### Step C. Tert-butyl N-{1-[3-(benzyloxy)benzenesulfonyl]piperidin-4-yl}carbamate (132-3)

A solution of 132-2 (6 g, 16.833 mmol, 1 equiv), benzyl bromide (5.76 g, 33.666 mmol, 2 equiv) and KOH (3.78 g, 67.332 mmol, 4 equiv) in MeCN was stirred for 3 h at room temperature. Desired product could be detected by LCMS. The reaction was quenched with water at 0° C. The aqueous layer was extracted with EtOAc (3×30 mL). The combined organic layers were dried over anhydrous Na₂SO₄. After filtration, the filtrate was concentrated under reduced pressure. The residue was purified by silica gel column chromatography, eluted with PE/EA (5:1) to afford 132-3 (6.5 g, 86.47%) as a white solid. LCMS (ESI, m/z): [M+H]⁺=447.

### Step D. 1-[3-(Benzyloxy)benzenesulfonyl]piperidin-4-amine (132-4)

A solution of 132-3 (5.5 g, 12.316 mmol, 1 equiv) and TFA (20 mL) in DCM (100 mL) was stirred for 2 h at room temperature. Desired product could be detected by LCMS. The reaction was quenched with sat. NaHCO₃ (aq.) at 0° C. The aqueous layer was extracted with DCM. The combined organic layers were dried over anhydrous Na₂SO₄ and concentrated under reduced pressure. The residue was purified by reversed-phase Combi-Flash to afford 132-4 (2 g, 46.87%) as a white solid. LCMS (ESI, m/z): [M+H]⁺=347.

### Step E. 2′-({1-[3-(Benzyloxy)benzenesulfonyl]piperidin-4-yl}amino)-7′-[(1R,3R)-3-(oxan-2-yloxy)cyclohexyl]spiro[cyclopropane-1,5′-pyrrolo[2,3-d]pyrimidin]-6′-one (132-5)

To a solution of 132-4 (1.5 g, 4.330 mmol, 1 equiv), Int-5 (1.80 g, 4.763 mmol, 1.1 equiv) and 3rd Generation RuPhos precatalyst (0.72 g, 0.866 mmol, 0.2 equiv) in dioxane (10 mL) were added BINAP (0.54 g, 0.866 mmol, 0.2 equiv) and Cs₂CO₃ (5.64 g, 17.320 mmol, 4 equiv) at room temperature. The resulting mixture was stirred for 2 h at 90° C. under nitrogen atmosphere. The reaction was monitored by LCMS. The resulting mixture was concentrated under reduced pressure. The residue was purified by silica gel column chromatography, eluted with PE/EA (1:1) to afford 132-5 (1.1 g, 36.94%) as a yellow solid. LCMS (ESI, m/z): [M+H]⁺=688.

### Step F. 2′-{[1-(3-Hydroxybenzenesulfonyl)piperidin-4-yl]amino}-7′-[(1R,3R)-3-(oxan-2-yloxy)cyclohexyl]spiro[cyclopropane-1,5′-pyrrolo[2,3-d]pyrimidin]-6′-one (132-6)

To a solution of 132-5 (1.2 g, 1.745 mmol, 1 equiv) in CF₃CH₂OH (20 mL) was added Pd/C (10%, 1 g) under nitrogen atmosphere in a round-bottom flask. The mixture was hydrogenated at room temperature for 3 h under hydrogen atmosphere using a hydrogen balloon, filtered through a Celite pad and concentrated under reduced pressure. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. This resulted in 132-6 (800 mg, 76.72%) as a white solid. LCMS (ESI, m/z): [M+H]⁺=598.

### Step G. Tert-butyl 3-[3-(4-{7′-[(1R,3R)-3-(oxan-2-yloxy)cyclohexyl]-6′-oxospiro[cyclopropane-1,5′-pyrrolo[2,3-d]pyrimidin]-2′-ylamino}piperidin-1-ylsulfonyl)phenoxy]azetidine-1-carboxylate (132-7)

To a stirred solution of 132-6 (350 mg, 0.586 mmol, 1 equiv) and tert-butyl 3-hydroxyazetidine-1-carboxylate (121.71 mg, 0.703 mmol, 1.2 equiv) in Toluene (5 mL) were added DEAD (152.97 mg, 0.879 mmol, 1.5 equiv) and PPh₃ (307.17 mg, 1.172 mmol, 2 equiv) at 0° C. under nitrogen atmosphere. The resulting mixture was stirred for 1 h at 80° C. under nitrogen atmosphere. The reaction was quenched with water. The resulting mixture was extracted with EtOAc (3×15 mL). The combined organic layers were washed with brine, dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 76% gradient in 30 min; detector, UV 254 nm. The resulting mixture was concentrated under reduced pressure to afford 132-7 (320 mg, 72.58%) as a white solid. LCMS (ESI, m/z): [M+H]⁺=753.

### Step H. 2′-({1-[3-(Azetidin-3-yloxy)benzenesulfonyl]piperidin-4-yl}amino)-7′-[(1R,3R)-3-hydroxycyclohexyl]spiro[cyclopropane-1,5′-pyrrolo[2,3-d]pyrimidin]-6′-one (132-8)

To a stirred solution of 132-7 (320 mg, 0.425 mmol, 1 equiv) in MeCN (6 mL) was added 4M HCl (gas) in 1,4-dioxane (3 mL) dropwise at 0° C. The resulting mixture was stirred for 1 h at 0° C. Desired product could be detected by LCMS. The mixture was basified to pH 7 with Et₃N. The resulting mixture was concentrated under reduced pressure. The residue was purified by reversed-phase flash chromatography with the following conditions: column, C18 silica gel; mobile phase, MeCN in Water (0.1% FA), 10% to 30% gradient in 30 min; detector, UV 254 nm. The resulting mixture was concentrated under reduced pressure to afford 132-8 (100 mg, 41.37%) as a white solid. LCMS (ESI, m/z): [M+H]⁺=569.

### Step I. 3-[5-({3-[3-(4-{7′-[(1R,3R)-3-Hydroxycyclohexyl]-6′-oxospiro[cyclopropane-1,5′-pyrrolo[2,3-d]pyrimidin]-2′-ylamino}piperidin-1-ylsulfonyl)phenoxy]azetidin-1-yl}methyl)-1-oxo-3H-isoindol-2-yl]piperidine-2,6-dione (132)

To a stirred solution of 132-8 (50 mg, 0.088 mmol, 1 equiv) and Int-31 (47.87 mg, 0.176 mmol, 2 equiv) in DMF (1.5 mL) were added AcOH (2.64 mg, 0.044 mmol, 0.5 equiv) and STAB (37.27 mg, 0.176 mmol, 2 equiv) at room temperature under nitrogen atmosphere. The resulting mixture was stirred for 2 h at 50° C. under nitrogen atmosphere. Desired product could be detected by LCMS. The residue was purified by reversed-phase flash chromatography with the following conditions: column, C18 silica gel; mobile phase, MeCN in Water (10 mmol/L NH₄HCO₃), 10% to 35% gradient in 30 min; detector, UV 254 nm. The resulting mixture was concentrated under reduced pressure to afford compound 132 (17.8 mg, 23.88%) as a white solid. LCMS (ESI, m/z): [M+H]⁺=825.4.

### Example 133: 3-[5-(4-{1-[3-(4-{7′-[(1S,3S)-3-Hydroxycyclohexyl]-6′-oxospiro[cyclopropane-1,5′-pyrrolo[2,3-d] pyrimidin]-2′-ylamino}piperidin-1-ylsulfonyl)phenyl]azetidin-3-yl}piperazin-1-yl)-1-oxo-3H-isoindol-2-yl]piperidine-2,6-dione (133)

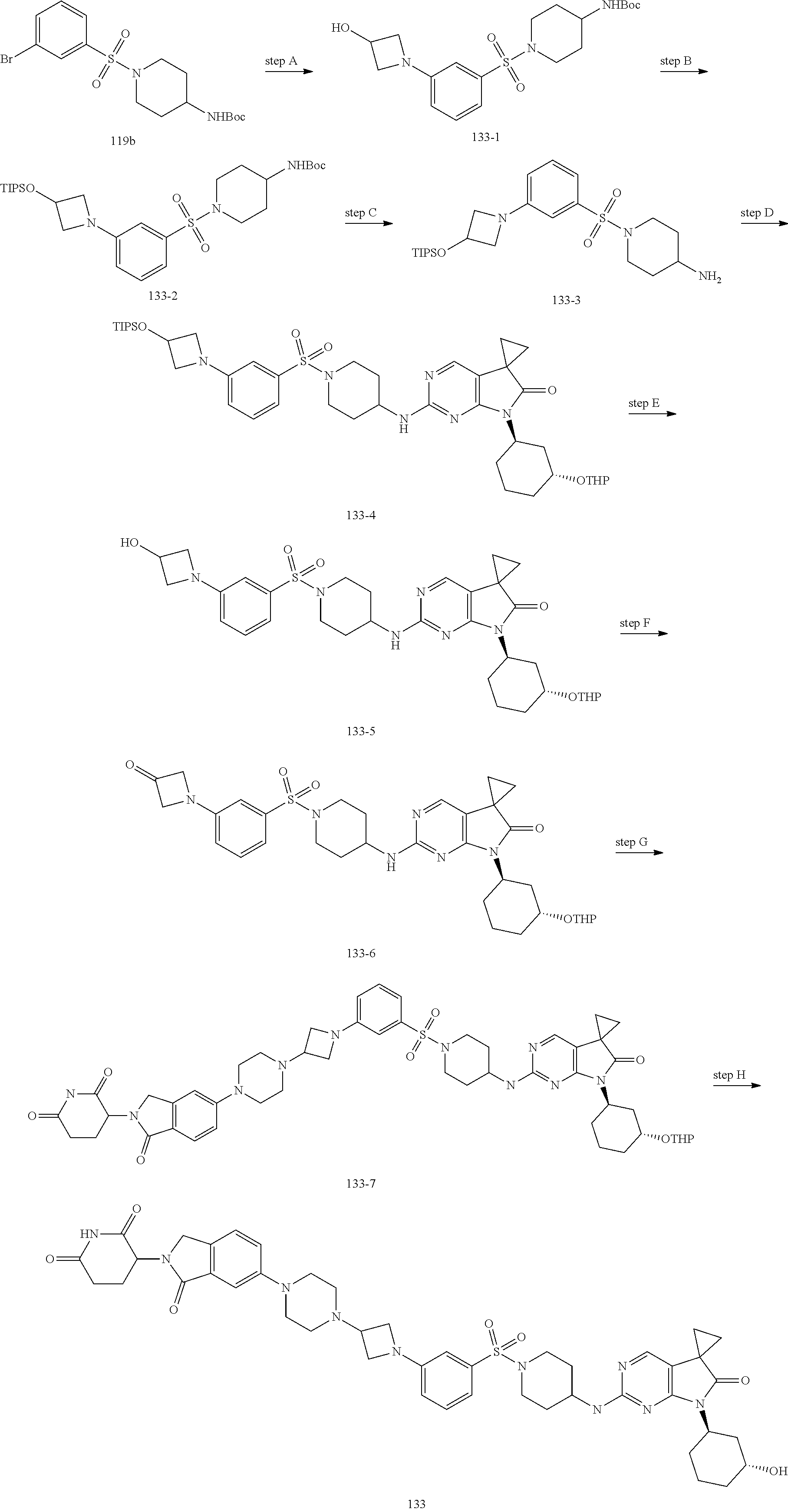

### Step A. Tert-butyl N-{1-[3-(3-hydroxyazetidin-1-yl)benzenesulfonyl]piperidin-4-yl}carbamate (133-1)

To a mixture of 119b (2 g, 4.770 mmol, 1 equiv), azetidin-3-ol (0.35 g, 4.770 mmol, 1 equiv) and CuI (0.18 g, 0.954 mmol, 0.2 equiv), L-proline (0.16 g, 1.431 mmol, 0.3 equiv) in DMSO (10 mL) were added K₂CO₃ (1.98 g, 14.310 mmol, 3 equiv) at 25 degrees C. under N₂. The resulting mixture was stirred for 12 h at 90 degrees C. Desired product could be detected by LCMS. The resulting mixture was diluted with water (50 mL) and extracted with EtOAc (3×100 mL). The combined organic layers were dried over anhydrous Na₂SO₄ and concentrated under reduced pressure. The residue was purified by reversed-phase Combi-Flash to afford 133-1 (1.4 g, 67.76%) as a yellow solid. LCMS (ESI, m/z): [M+H]⁺=412

### Step B. Tert-butyl N-[1-(3-{3-[(triisopropylsilyl)oxy]azetidin-1-yl}benzenesulfonyl)piperidin-4-yl]carbamate (133-2)

To a mixture of 133-1 (2.1 g, 5.103 mmol, 1 equiv), chlorotriisopropylsilane (1.97 g, 10.206 mmol, 2 equiv) and DMAP (0.06 g, 0.510 mmol, 0.1 equiv) in DCM (10 mL) were added Imidazole (0.69 g, 10.206 mmol, 2 equiv) at 25 degrees C. The resulting mixture was stirred for 12 h at 25 degrees C. Desired product could be detected by LCMS. The mixture was concentrated under vacuum. The residue was purified by reversed-phase Combi-Flash to afford 133-2 (2.2 g, 70.61%) as a yellow solid. LCMS (ESI, m/z): [M+H]⁺=568.

### Step C. 1-(3-{3-[(Triisopropylsilyl)oxy]azetidin-1-yl}benzenesulfonyl)piperidin-4-amine (133-3)

To a stirred solution of 133-2 (2.1 g, 3.698 mmol, 1 equiv) in DCM (20 mL) was added TFA (12.65 g, 110.940 mmol, 30 equiv) at 0 degrees C. The resulting mixture was stirred for 1 h at 25 degrees C. Desired product could be detected by LCMS. The resulting mixture was diluted with sat. NaHCO₃ (aq.) and extracted with DCM (3×20 mL). The combined organic layers were dried over anhydrous Na₂SO₄ and concentrated under reduced pressure. The residue was purified by reversed-phase Combi-Flash to afford 133-3 (1.8 g, 89.49%) as a white solid. LCMS (ESI, m/z): [M+H]⁺=468.

### Step D. 7′-[(1R,3R)-3-(oxan-2-yloxy)cyclohexyl]-2′-{[1-(3-{3-[(triisopropylsilyl)oxy]azetidin-1-yl}benzenesulfonyl)piperidin-4-yl]amino}spiro[cyclopropane-1,5′-pyrrolo[2,3-d]pyrimidin]-6′-one (133-4)

To a mixture of 133-3 (800 mg, 1.710 mmol, 1 equiv), Int-5 (646.29 mg, 1.710 mmol, 1 equiv) and {1,3-bis[2,6-bis(pentan-3-yl)phenyl]-2,3-dihydro-1H-imidazol-2-yl}dichloro(3-chloropyridin-1-ium-1-yl)palladium (135.58 mg, 0.171 mmol, 0.1 equiv) in dioxane (10 mL) were added Cs₂CO₃ (1671.80 mg, 5.130 mmol, 3 equiv) at 25 degrees C. under N₂. The resulting mixture was stirred for 12 h at 100 degrees C. The resulting mixture was diluted with water and extracted with EtOAc (3×100 mL). The combined organic layers were dried over anhydrous Na₂SO₄ and concentrated under reduced pressure. The residue was purified by reversed-phase Combi-Flash to afford 133-4 (650 mg, 45.09%) as a yellow solid. LCMS (ESI, m/z): [M+H]⁺=809.

### Step E. 2′-({1-[3-(3-hydroxyazetidin-1-yl)benzenesulfonyl]piperidin-4-yl}amino)-7′-[(1R,3R)-3-(oxan-2-yloxy)cyclohexyl]spiro[cyclopropane-1,5′-pyrrolo[2,3-d]pyrimidin]-6′-one (133-5)

To a solution of 133-4 (600 mg, 0.742 mmol, 1 equiv) in DMF (10 mL) was added CsF (1.13 g, 7.420 mmol, 10 equiv) at 25 degrees C. The resulting mixture was stirred for 1 h at 25 degrees C. The resulting mixture was diluted with water and extracted with EtOAc (3×50 mL). The combined organic layers were dried over anhydrous Na₂SO₄ and concentrated under reduced pressure. The residue was purified by reversed-phase Combi-Flash to afford 133-5 (500 mg, 83.67%) as a white solid. LCMS (ESI, m/z): [M+H]⁺=653.

### Step F. 7′-[(1S,3S)-3-Oxan-2-yloxy)cyclohexyl]-2′-({1-[3-(3-oxoazetidin-1-yl)benzenesulfonyl]piperidin-4-yl}amino)spiro[cyclopropane-1,5′-pyrrolo[2,3-d]pyrimidin]-6′-one (133-6)

To a stirred solution of 133-5 (500 mg, 0.766 mmol, 1 equiv) in DCM (10 mL) was added Dess-Martin (487.29 mg, 1.149 mmol, 1.5 equiv) at 25 degrees C. The resulting mixture was stirred for 1 h at 25 degrees C. The resulting mixture was diluted with water and extracted with EtOAc (3×10 mL). The combined organic layers were dried over anhydrous Na₂SO₄ and concentrated under reduced pressure. The residue was purified by reversed-phase Combi-Flash to afford 133-6 (300 mg, 48.75%) as a white solid. LCMS (ESI, m/z): [M+H]⁺=651.

### Step G. 3-[5-(4-{1-[3-(4-{7′-[(1S,3S)-3-(Oxan-2-yloxy)cyclohexyl]-6′-oxospiro[cyclopropane-1,5′-pyrrolo[2,3-d]pyrimidin]-2′-ylamino}piperidin-1-ylsulfonyl)phenyl]azetidin-3-yl}piperazin-1-yl)-1-oxo-3H-isoindol-2-yl]piperidine-2,6-dione (133-7)

To a mixture of 133-6 (200 mg, 0.307 mmol, 1 equiv), Int-29 (153.86 mg, 0.307 mmol, 1 equiv) and AcOH (184.55 mg, 3.070 mmol, 10 equiv), Titanium(IV) ethoxide (701.01 mg, 3.070 mmol, 10 equiv) in DMF (10 mL) were added STAB (195.40 mg, 0.921 mmol, 3 equiv) at 25 degrees C. under N₂. The resulting mixture was stirred for 1 h at 25 degrees C. The resulting mixture was diluted with water (20 mL) and extracted with EtOAc (3×50 mL). The combined organic layers were dried over anhydrous Na₂SO₄ and concentrated under reduced pressure. The residue was purified by silica gel column chromatography, eluted with MeOH/DCM (1:10) to afford 133-7 (130 mg, 36.01%) as a yellow solid. LCMS (ESI, m/z): [M+H]⁺=963.

### Step H. 3-[5-(4-{1-[3-(4-{7′-[(1S,3S)-3-Hydroxycyclohexyl]-6′-oxospiro[cyclopropane-1,5′-pyrrolo[2,3-d]pyrimidin]-2′-ylamino}piperidin-1-ylsulfonyl)phenyl]azetidin-3-yl}piperazin-1-yl)-1-oxo-3H-isoindol-2-yl]piperidine-2,6-dione (133)

To a stirred solution of 133-7 (80 mg, 0.083 mmol, 1 equiv) in DCM (5 mL) was added 4M HCl (gas) in 1,4-dioxane (1 mL) at 0 degrees C. The resulting mixture was stirred for 1 h at 25 degrees C. The reaction was quenched by the addition of TEA (1 mL) at 0° C. The resulting mixture was concentrated under reduced pressure. The crude product was purified by reverse phase flash to afford compound 133 (19.2 mg, 25.22%) as a white solid. LCMS (ESI, m/z): [M+H]⁺=879.5.

### Example 134: 3-[5-(4-{3-[3-(4-{7′-[(1R,3R)-3-Hydroxycyclohexyl]-6′-oxospiro[cyclopropane-1,5′-pyrrolo[2,3-d]pyrimidin]-2′-ylamino}piperidin-1-ylsulfonyl)phenoxy]azetidin-1-yl}piperidin-1-yl)-1-oxo-3H-isoindol-2-yl]piperidine-2,6-dione (134)

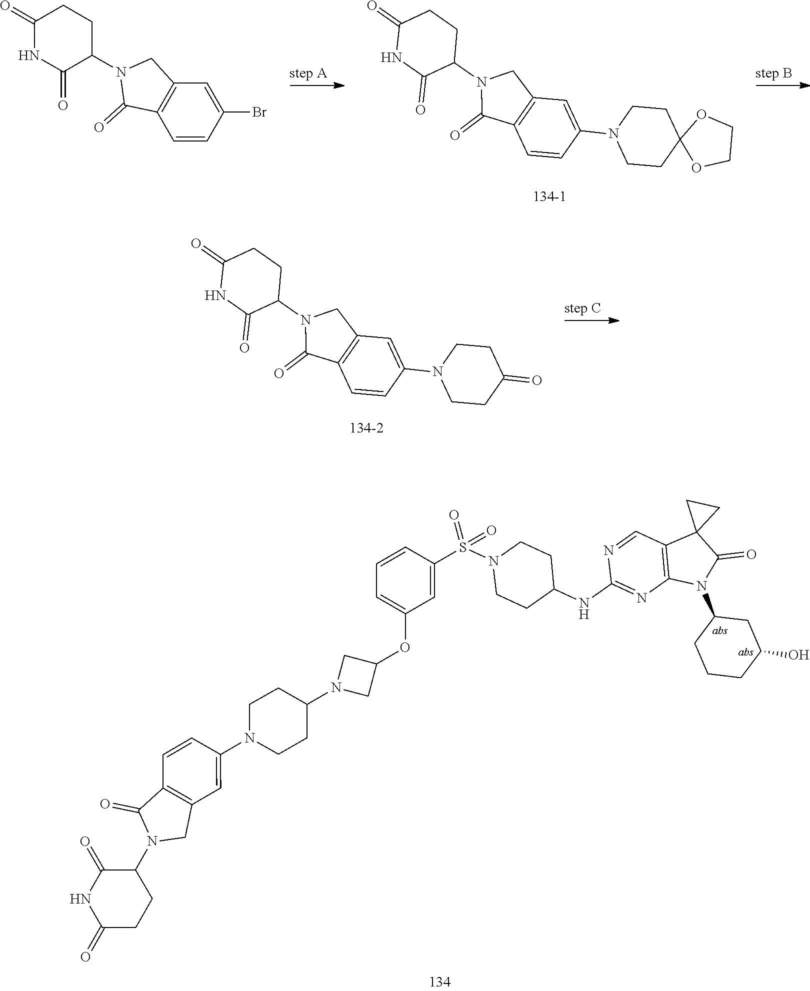 [SMILES: O=C1CCC(N2Cc3cc(Br)ccc3C2=O)C(=O)N1 ; O=C1CCC(N2Cc3cc(N4CCC5(CC4)OCCO5)ccc3C2=O)C(=O)N1 ; O=C1CCN(c2ccc3c(c2)CN(C2CCC(=O)NC2=O)C3=O)CC1 ; O=C1CCC(N2Cc3cc(N4CCC(N5CC(Oc6cccc(S(=O)(=O)N7CCC(Nc8ncc9c(n8)N([C@@H]8CCC[C@@H](O)C8)C(=O)C98CC8)CC7)c6)C5)CC4)ccc3C2=O)C(=O)N1]

### Step A. 3-(5-{1,4-Dioxa-8-azaspiro[4.5]decan-8-yl}-1-oxo-3H-isoindol-2-yl)piperi dine-2,6-dione (134-1)

To a mixture of 3-(5-bromo-1-oxo-3H-isoindol-2-yl)piperidine-2,6-dione (1 g, 3.095 mmol, 1 equiv) and 1,4-dioxa-8-azaspiro[4.5]decane (531.72 mg, 3.714 mmol, 1.2 equiv) in DMF (10 mL) was added Cs₂CO₃ (4033.09 mg, 12.380 mmol, 4 equiv) and RuPhos Palladacycle Gen.3 (388.24 mg, 0.464 mmol, 0.15 equiv). The resulting mixture was stirred for 16 h at 100° C. The reaction was quenched by the addition of water. The resulting mixture was extracted with CH₂Cl₂. The combined organic layers were dried over anhydrous Na₂SO₄. After filtration, the filtrate was concentrated under reduced pressure. The residue was purified by silica gel column chromatography, eluted with PE/EA (10:1) to afford 134-1 (300 mg, 23.39%) as a white solid. LCMS (ESI, m/z): [M+H]⁺=386

### Step B. 3-[1-Oxo-5-(4-oxopiperidin-1-yl)-3H-isoindol-2-yl]piperidine-2,6-dione (134-2)

To a stirred mixture of 134-1 (180 mg, 0.467 mmol, 1 equiv) in DCM (4.5 mL) was added concentrated HCl (4.5 mL). The resulting mixture was stirred for 1 h at 50° C. The reaction was quenched by the addition of water (10 mL). The resulting mixture was extracted with CH₂Cl₂. The combined organic layers were dried over anhydrous Na₂SO₄. After filtration, the filtrate was concentrated under reduced pressure to afford 134-2 (150 mg, 76.21%) as a white solid. LCMS (ESI, m/z): [M+H]⁺=342.

### Step C. 3-[5-(4-{3-[3-(4-{7′-[(1R,3R)-3-Hydroxycyclohexyl]-6′-oxospiro[cyclopropane-1,5′-pyrrolo[2,3-d]pyrimidin]-2′-ylamino}piperidin-1-ylsulfonyl)phenoxy]azetidin-1-yl}piperidin-1-yl)-1-oxo-3H-isoindol-2-yl]piperidine-2,6-dione (134)

Compound 134 was prepared from 134-2 and 132-8 following the procedure for the synthesis of compound 132 in example 132. LCMS (ESI, m/z): [M+H]⁺=894.5.

### Example 135: 3-[5-(4-{3-[3-(4-{7′-[(1R,3R)-3-Hydroxycyclohexyl]-6′-oxospiro[cyclopropane-1,5′-pyrrolo[2,3-d]pyrimidin]-2′-ylamino}piperidin-1-ylsulfonyl)phenoxy]azetidin-1-yl}piperidin-1-yl)-1-oxo-3H-isoindol-2-yl]piperidine-2,6-dione (135)

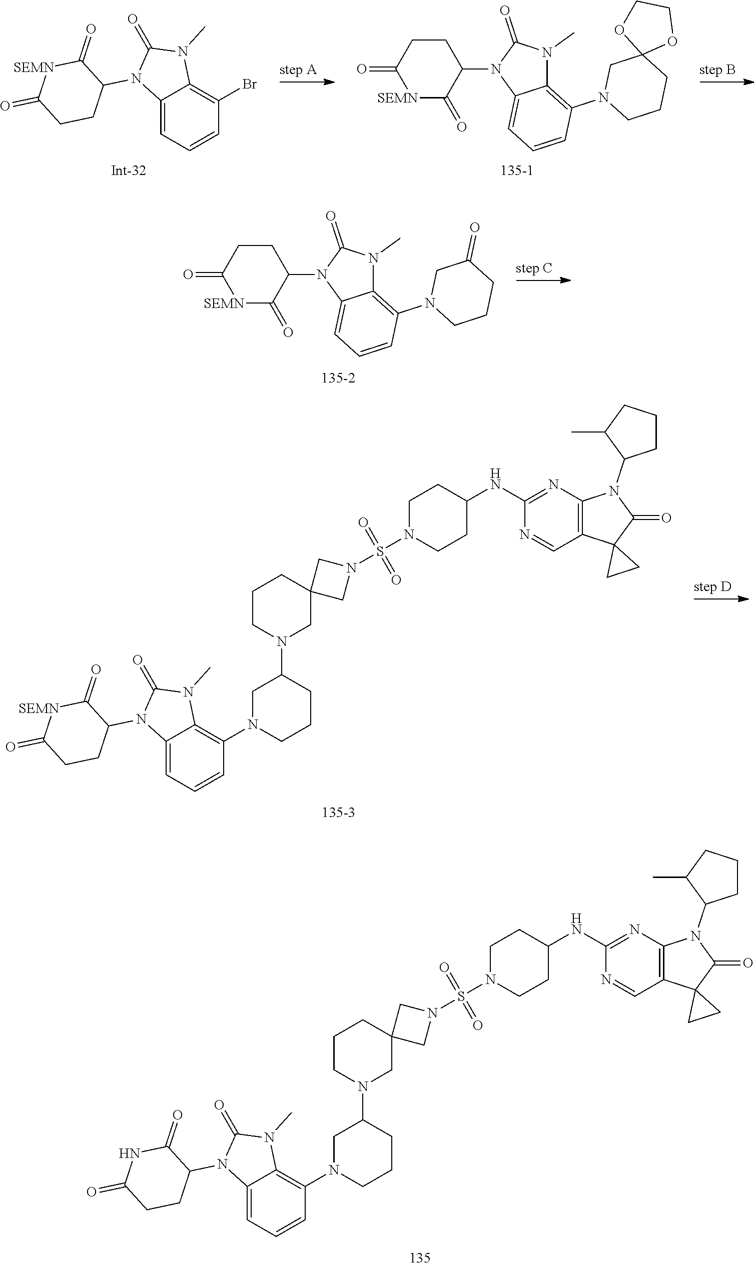 [SMILES: Cn1c(=O)n(C2CCC(=O)N(COCC[Si](C)(C)C)C2=O)c2cccc(Br)c21 ; Cn1c(=O)n(C2CCC(=O)N(COCC[Si](C)(C)C)C2=O)c2cccc(N3CCCC4(C3)OCCO4)c21 ; Cn1c(=O)n(C2CCC(=O)N(COCC[Si](C)(C)C)C2=O)c2cccc(N3CCCC(=O)C3)c21 ; CC1CCCC1N1C(=O)C2(CC2)c2cnc(NC3CCN(S(=O)(=O)N4CC5(CCCN(C6CCCN(c7cccc8c7n(C)c(=O)n8C7CCC(=O)N(COCC[Si](C)(C)C)C7=O)C6)C5)C4)CC3)nc21 ; CC1CCCC1N1C(=O)C2(CC2)c2cnc(NC3CCN(S(=O)(=O)N4CC5(CCCN(C6CCCN(c7cccc8c7n(C)c(=O)n8C7CCC(=O)NC7=O)C6)C5)C4)CC3)nc21]

### Step A. 3-(4-{1,4-Dioxa-7-azaspiro[4.5]decan-7-yl}-3-methyl-2-oxo-1,3-benzodiazol-1-yl)-1-{[2-(trimethylsilyl)ethoxy]methyl}piperidine-2,6-dione (135-1)

A solution of 1,4-dioxa-7-azaspiro[4.5]decane (500 mg, 3.492 mmol, 1 equiv), Int-32 (1635.72 mg, 3.492 mmol, 1 equiv) in dioxane (5 mL) was treated with Pd-PEPPSI-IPentCl 2-methylpyridine (o-picoline) (293.73 mg, 0.349 mmol, 0.1 equiv) at 25° C. under nitrogen atmosphere followed by the addition of K₂CO₃ (1447.82 mg, 10.476 mmol, 3 equiv) at 25° C. The resulting mixture was stirred for 16 h at 100° C. under nitrogen atmosphere. The resulting mixture was concentrated under reduced pressure. The residue was purified by silica gel column chromatography, eluted with petroleum ether/EtOAc (1:1) to afford 135-1 (620 mg, 33.46% yield) as a white solid. LCMS (ESI, m/z): [M+H]⁺=531.

### Step B. 3-[3-Methyl-2-oxo-4-(3-oxopiperidin-1-yl)-1,3-benzodiazol-1-yl]-1-{[2-(trimethylsilyl)ethoxy]methyl}piperidine-2,6-dione (135-2)

A solution of 135-1 (300 mg, 0.565 mmol, 1 equiv) in acetone (6 mL) was treated with TsOH (389.37 mg, 2.260 mmol, 4 equiv) and H₂O (3 mL) at 25° C. under nitrogen atmosphere. The resulting mixture was stirred for 2 h at 60° C. under nitrogen atmosphere. The resulting mixture was concentrated under reduced pressure. The residue was purified by Prep-TLC (petroleum ether/EtOAc 1:1) to afford 135-2 (130 mg, 47.26% yield) as a white solid. LCMS (ESI, m/z): [M+H]⁺=487.

### Step C. 3-{3-Methyl-4-[3-(2-{4-[7′-(2-methylcyclopentyl)-6′-oxospiro[cyclopropane-1,5′-pyrrolo[2,3-d]pyrimidin]-2′-ylamino]piperidin-1-ylsulfonyl}-2,6-diazaspiro[3.5]nonan-6-yl)piperidin-1-yl]-2-oxo-1,3-benzodiazol-1-yl}-1-{[2-(trimethylsilyl)ethoxy]methyl}piperidine-2,6-dione (135-3)

A solution of 135-2 (150 mg, 0.308 mmol, 1 equiv) in DMF (2 mL) was treated with Int-15 (163.27 mg, 0.308 mmol, 1 equiv) at 25° C. under nitrogen atmosphere followed by the addition of AcOH (5.55 mg, 0.092 mmol, 0.3 equiv) dropwise at 25° C. The resulting mixture was stirred for 1 h at 50° C. under nitrogen atmosphere. To the above mixture was added NaBH₃CN (58.11 mg, 0.924 mmol, 3 equiv) at 25° C. The resulting mixture was stirred for additional 16 h at 50° C. The resulting mixture was diluted with water and extracted with EtOAc. The combined organic layers were dried over anhydrous Na₂SO₄ and concentrated under reduced pressure. The residue was purified by Prep-TLC (petroleum ether/EtOAc 1:1) to afford 135-3 (80 mg, 25.95% yield) as a white solid. LCMS (ESI, m/z): [M+H]⁺=1001.

### Step D. 3-{3-Methyl-4-[3-(2-{4-[7′-(2-methylcyclopentyl)-6′-oxospiro[cyclopropane-1,5′-pyrrolo[2,3-d]pyrimidin]-2′-ylamino]piperidin-1-ylsulfonyl}-2,6-diazaspiro[3.5]nonan-6-yl)piperidin-1-yl]-2-oxo-1,3-benzodiazol-1-yl}piperidine-2,6-dione (135)

A solution of 135-3 (50 mg, 0.050 mmol, 1 equiv) in DCM (1 mL) was treated with TFA (170.97 mg, 1.500 mmol, 30 equiv) at 0° C. under nitrogen atmosphere. The resulting mixture was stirred for 1 h at 25° C. under nitrogen atmosphere. To the above mixture was added Et₃N (303.47 mg, 3.000 mmol, 60 equiv) and DMEDA (5.29 mg, 0.060 mmol, 1.2 equiv) dropwise over 1 min at 0° C. The resulting mixture was stirred for additional 0.5 h at 25° C. The residue was purified by reversed-phase flash chromatography with the following conditions: column, C18 silica gel; mobile phase, MeCN in Water (0.1% NH₄HCO₃), 60% to 70% gradient in 10 min; detector, UV 220 nm. to afford compound 135 (12.2 mg, 27.86% yield) as a white solid. LCMS (ESI, m/z): [M+H]⁺=870.4.

### Example 136: 3-{3-Methyl-4-[3-(2-{4-[7′-(2-methylcyclopentyl)-6′-oxospiro[cyclopropane-1,5′-pyrrolo[2,3-d]pyrimidin]-2′-ylamino]piperidin-1-ylsulfonyl}-2,6-diazaspiro[3.5]nonan-6-yl)phenyl]-2-oxo-1,3-benzodiazol-1-yl}piperidine-2,6-dione (136)

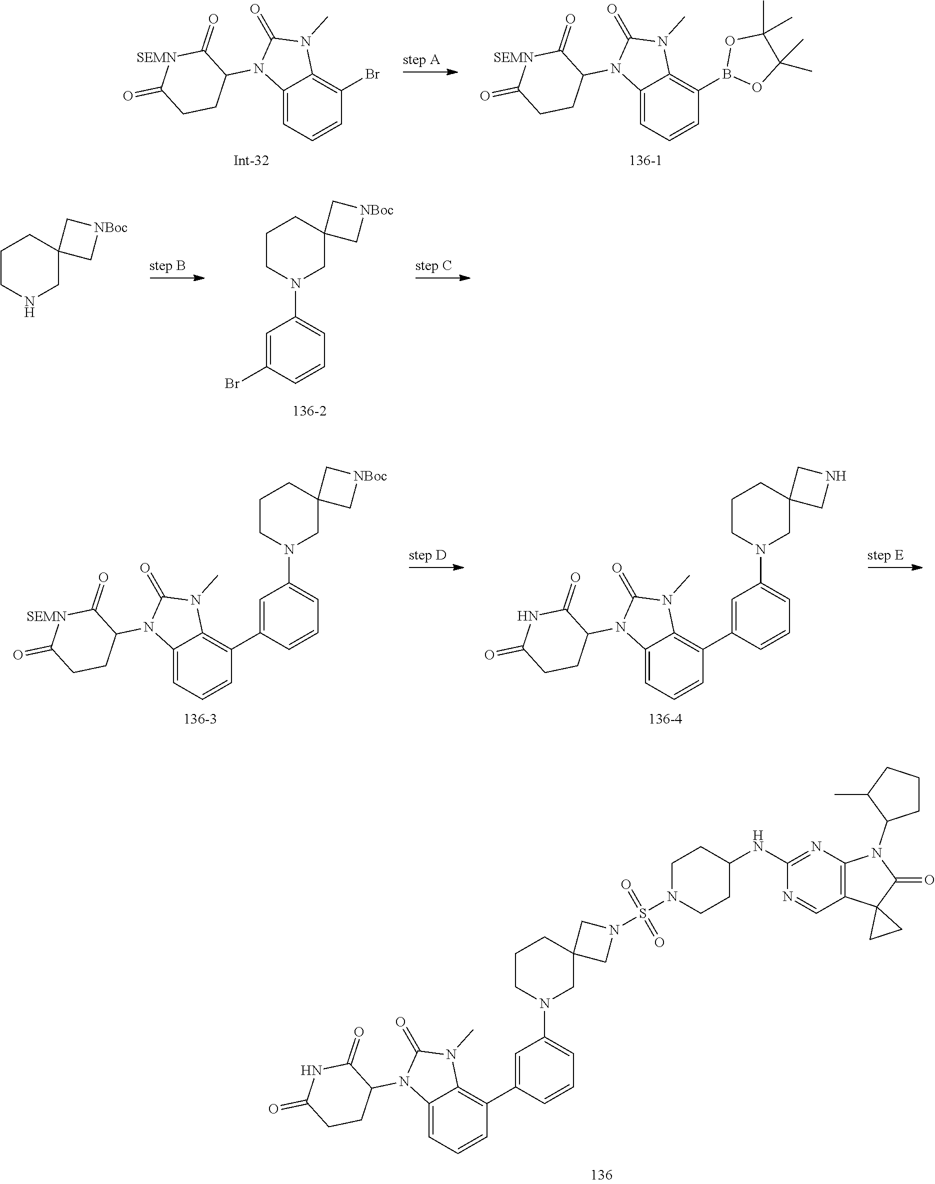

### Step A. 3-[3-Methyl-2-oxo-4-(4,4,5,5-tetramethyl-1,3,2-dioxaborolan-2-yl)-1,3-benzodiazol-1-yl]-1-{[2-(trimethylsilyl)ethoxy]methyl}piperidine-2,6-dione (136-1)

To a stirred mixture of Int-32 (2.5 g, 5.337 mmol, 1 equiv) and bis(pinacolato)diboron (2.03 g, 8.005 mmol, 1.5 equiv) in dioxane (25 mL) were added KOAC (1.05 g, 10.674 mmol, 2 equiv) and Dichlorobis(triphenylphosphine)palladium(II) (0.37 g, 0.534 mmol, 0.1 equiv) at room temperature under argon atmosphere. The resulting mixture was stirred for 2 h at 80° C. under argon atmosphere. The resulting mixture was concentrated under reduced pressure. The residue was purified by silica gel column chromatography, eluted with PE/EA (1:1) to afford 136-1 (2 g, 72.70%) as a white solid. LCMS (ESI, m/z): [M+H]⁺=516.

### Step B. Tert-butyl 6-(3-bromophenyl)-2,6-diazaspiro[3.5]nonane-2-carboxylate (136-2)

To a solution of tert-butyl 2,6-diazaspiro[3.5]nonane-2-carboxylate (200 mg, 0.882 mmol, 1 equiv) and Pd₂(dba)₃ (80.92 mg, 0.088 mmol, 0.1 equiv), BINAP (55.01 mg, 0.088 mmol, 0.1 equiv), t-BuONa (169.88 mg, 1.76 mmol, 2 equiv) in Toluene (4 mL) was added benzene, 1-bromo-3-iodo- (375.0 mg, 1.32 mmol, 1.5 equiv) at room temperature under argon atmosphere. The resulting mixture was stirred for 1 h at 90° C. under argon atmosphere. Desired product could be detected by LCMS. The resulting mixture was extracted with EtOAc and water. The combined organic layers were dried over anhydrous Na₂SO₄. After filtration, the filtrate was concentrated under reduced pressure. The residue was purified by Prep-TLC (PE/EA 5:1) to afford 136-2 (150 mg, 44.51%) as a yellow oil. LCMS (ESI, m/z): [M+H]⁺=381.

### Step C. Tert-butyl 6-{3-[1-(2,6-dioxo-1-{1-[2-(trimethylsilyl)ethoxy]methyl}piperidin-3-yl)-3-methyl-2-oxo-1,3-benzodiazol-4-yl]phenyl}-2,6-diazaspiro[3.5]nonane-2-carboxylate (136-3)

To a solution of 136-2 (100 mg, 0.262 mmol, 1 equiv) and 136-1 (202.78 mg, 0.393 mmol, 1.5 equiv) in dioxane (2.5 mL)/H₂O (0.25 mL) were added K₂CO₃ (72.49 mg, 0.524 mmol, 2 equiv) and Pd(DtBPF)Cl₂ (85.46 mg, 0.131 mmol, 0.5 equiv). After stirring for 1 h at 80° C. under a nitrogen atmosphere, the reaction mixture was quenched by addition of water. The aqueous layer was extracted with EtOAc. The combined organic phase was dried over anhydrous sodium sulfate and concentrated under reduced pressure to give crude product which was further purified by Prep-TLC (PE/EA 1/1) to afford 136-3 (160 mg, 88.43%) as a brown solid. LCMS (ESI, m/z): [M+H]⁺=670.

### Step D. 3-[4-(3-{2,6-Diazaspiro[3.5]nonan-6-yl}phenyl)-3-methyl-2-oxo-1,3-benzodiazol-1-yl]-1-{[2-(trimethylsilyl)ethoxy]methyl}piperidine-2,6-dione (136-4)

To a stirred solution of 136-3 (150 mg, 0.217 mmol, 1 equiv) in anhydrous ACN (1 mL) was added 4M HCl (gas) in 1,4-dioxane (1 mL) at 0° C. and stirred for 1 h. Desired product could be detected by LCMS. The resulting mixture was concentrated under reduced pressure. The residue was purified by reversed-phase flash chromatography with the following conditions: column, C18 silica gel; mobile phase, 0.1% NH₄HCO₃ in ACN, 20% to 45% gradient in 10 min; detector, UV 254 nm. This resulted in 136-4 (100 mg, 77.98%) as a white solid. LCMS (ESI, m/z): [M+H]⁺=460.

### Step E. 3-{3-Methyl-4-[3-(2-{4-[7′-(2-methylcyclopentyl)-6′-oxospiro[cyclopropane-1,5′-pyrrolo[2,3-d]pyrimidin]-2′-ylamino]piperidin-1-ylsulfonyl}-2,6-diazaspiro[3.5]nonan-6-yl)phenyl]-2-oxo-1,3-benzodiazol-1-yl}piperidine-2,6-dione (136)

To a stirred solution of 136-4 (80 mg, 0.174 mmol, 1 equiv), DIEA (674.99 mg, 5.220 mmol, 30 equiv) in anhydrous DCM (4 mL) was added 13-6 (84.25 mg, 0.191 mmol, 1.1 equiv) at 0° C. and stirred for 1 h. The resulting mixture was concentrated under reduced pressure. The residue was purified by reversed-phase flash chromatography with the following conditions: column, C18 silica gel; mobile phase, 0.1% FA in MeCN, 20% to 40% gradient in 10 min; detector, UV 254 nm. This resulted in compound 136 (12.1 mg, 7.86%) as a white solid. LCMS (ESI, m/z): [M+H]⁺=863.3.

### Example 138: 3-{3-Methyl-4-[2-(2-{4-[7′-(2-methylcyclopentyl)-6′-oxospiro[cyclopropane-1,5′-pyrrolo[2,3-d]pyrimidin]-2′-ylamino]piperidin-1-ylsulfonyl}-2,6-diazaspiro[3.5]nonan-6-yl)ethoxyl-2-oxo-1,3-benzodiazol-1-yl]piperidine-2,6-dione (138)

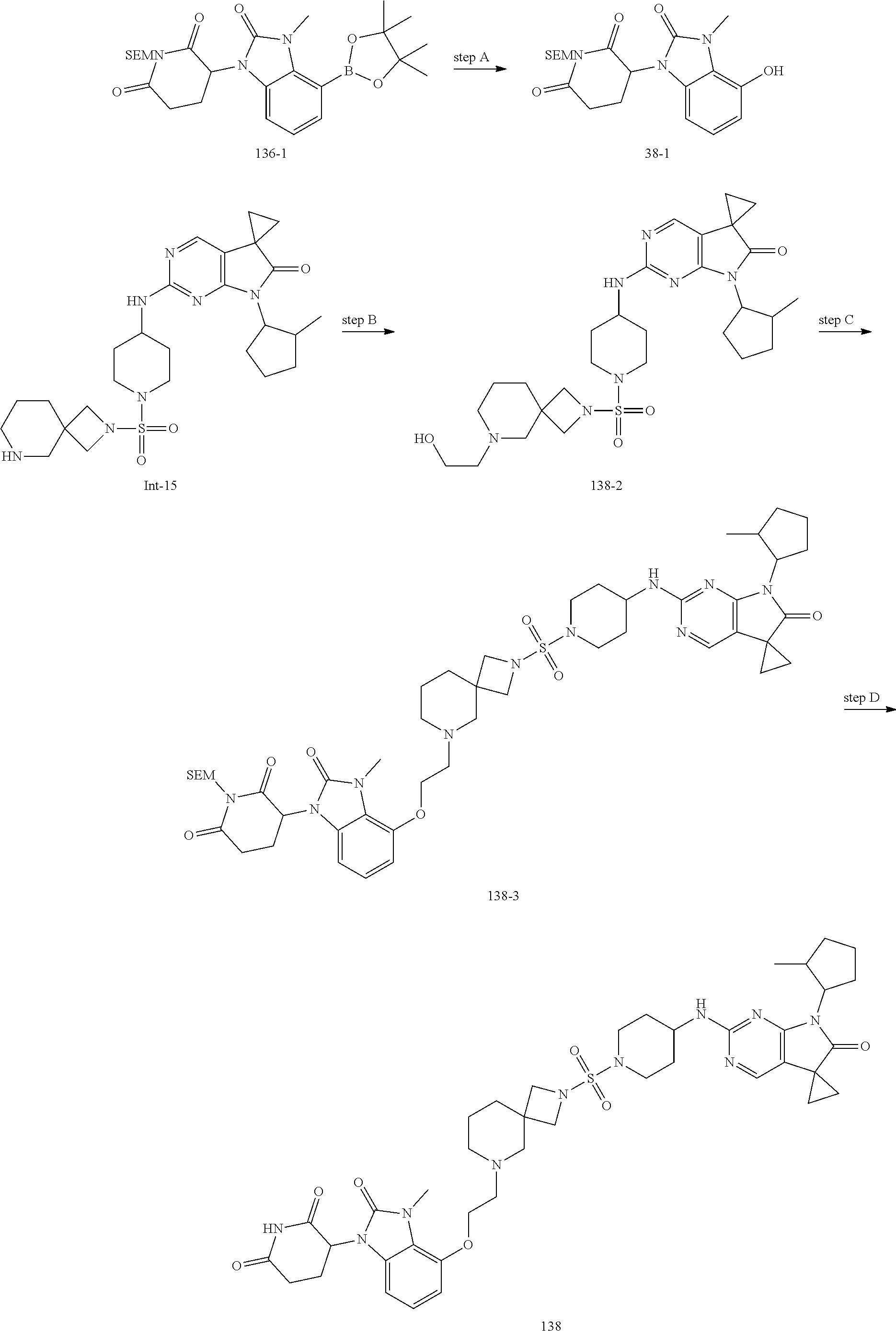

### Step A. 3-(4-Hydroxy-3-methyl-2-oxo-1,3-benzodiazol-1-yl)-1-{[2-(trimethylsilyl) ethoxy]methyl}piperidine-2,6-dione (138-1)

To a stirred mixture of 136-1 (3 g, 5.820 mmol, 1 equiv) in dioxane (20 mL) was added 30% H₂O₂ (2.62 g, 77.173 mmol, 13.26 equiv) at 0° C. The resulting mixture was stirred for 1 h at room temperature. The reaction was monitored by TLC. The resulting mixture was concentrated under reduced pressure. The residue was purified by silica gel column chromatography, eluted with PE/EA (1:1) to afford 138-1 (1.26 g, 53.39%) as a white solid. LCMS (ESI, m/z): [M+H]⁺=406.

### Step B. 2′-({1-[6-(2-Hydroxyethyl)-2,6-diazaspiro[3.5]nonan-2-ylsulfonyl]piperidin-4-yl}amino)-7′-(2-methylcyclopentyl)spiro[cyclopropane-1,5′-pyrrolo[2,3-d]pyrimidin]-6′-one (138-2)

To a solution of Int-15 (120 mg, 0.227 mmol, 1 equiv) in MeCN (2 mL) was added 2-bromoethanol (56.62 mg, 0.454 mmol, 2 equiv), K₂CO₃ (93.93 mg, 0.681 mmol, 3 equiv), KI (3.76 mg, 0.023 mmol, 0.1 equiv) at room temperature. The mixture was stirred for 1 hour at 60° C. Desired product could be detected by LCMS. The resulting mixture was concentrated under reduced pressure. The residue was purified by silica gel column chromatography, eluted with PE/EA (3/1) to afford 138-2 (80 mg, 61.55%) as a white solid. LCMS (ESI, m/z): [M+H]⁺=574.

### Step C. 3-{3-Methyl-4-[2-(2-{4-[7′-(2-methylcyclopentyl)-6′-oxospiro[cyclopropane-1,5′-pyrrolo[2,3-d]pyrimidin]-2′-ylamino]piperidin-1-ylsulfonyl}-2,6-diazaspiro[3.5]nonan-6-yl)ethoxy]-2-oxo-1,3-benzodiazol-1-yl}-1-{[2-(trimethylsilyl)ethoxy]methyl}piperidine-2,6-dione (138-3)

To a solution of 138-2 (65 mg, 0.113 mmol, 1 equiv) in Toluene (1 mL) was added 138-1 (45.94 mg, 0.113 mmol, 1 equiv), PPh₃ (104.00 mg, 0.396 mmol, 3.5 equiv), DEAD (29.59 mg, 0.170 mmol, 1.5 equiv). The mixture was stirred for 1 hour at 90° C. under N₂. Desired product could be detected by LCMS. The resulting mixture was concentrated under reduced pressure. The residue was purified by silica gel column chromatography, eluted with DCM/MeOH (30/1) to afford 138-3 (40 mg, 36.73%) as a white solid. LCMS (ESI, m/z): [M+H]⁺=961.

### Step D. 3-{3-Methyl-4-[2-(2-{4-[7′-(2-methylcyclopentyl)-6′-oxospiro[cyclopropane-1,5′-pyrrolo[2,3-d]pyrimidin]-2′-ylamino]piperidin-1-ylsulfonyl}-2,6-diazaspiro[3.5]nonan-6-yl)ethoxy]-2-oxo-1,3-benzodiazol-1-yl}piperidine-2,6-dione (138)

To a solution of 138-3 (40 mg, 0.042 mmol, 1 equiv) in DCM (1 mL) was added CF₃COOH (0.09 mL) at 0° C. The mixture was stirred for 1 h. Desired product could be detected by LCMS. The resulting mixture was concentrated under reduced pressure. The residue was purified by reversed-phase flash chromatography with the following conditions: column, C18 silica gel; mobile phase, H₂O in MeCN, 0% to 80% gradient in 35 min; detector, UV 254 nm. The resulting mixture was concentrated under reduced pressure to afford compound 138 (8.0 mg, 22.33%) as white solid. LCMS (ESI, m/z): [M+H]⁺=831.3.

### Example 140: 3-(3-Methyl-5-{4-[(3-{4-[7′-(2-methylcyclopentyl)-6′-oxospiro[cyclopropane-1,5′-pyrrolo[2,3-d]pyrimidin]-2′-ylamino]piperidin-1-ylsulfonyl}pyrrolidin-1-yl)methyl]piperidin-1-yl}-2-oxo-1,3-benzodiazol-1-yl)piperidine-2,6-dione (140)

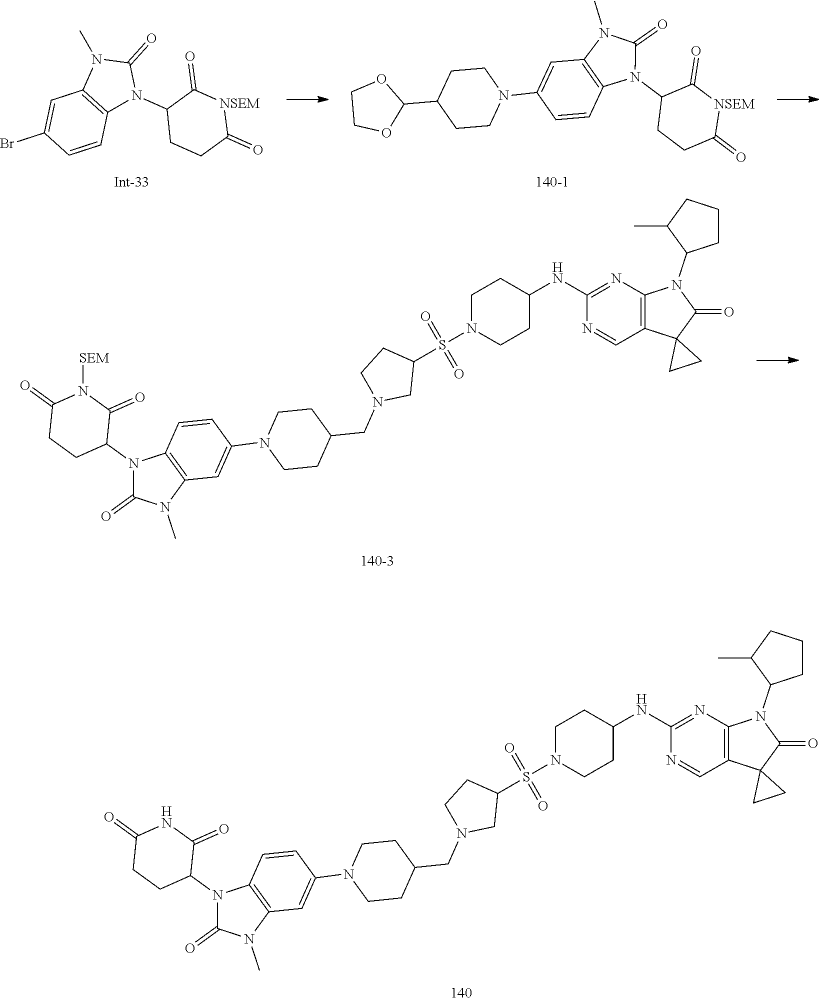 [SMILES: Cn1c(=O)n(C2CCC(=O)N(COCC[Si](C)(C)C)C2=O)c2ccc(Br)cc21 ; Cn1c(=O)n(C2CCC(=O)N(COCC[Si](C)(C)C)C2=O)c2ccc(N3CCC(C4OCCO4)CC3)cc21 ; CC1CCCC1N1C(=O)C2(CC2)c2cnc(NC3CCN(S(=O)(=O)C4CCN(CC5CCN(c6ccc7c(c6)n(C)c(=O)n7C6CCC(=O)N(COCC[Si](C)(C)C)C6=O)CC5)C4)CC3)nc21 ; CC1CCCC1N1C(=O)C2(CC2)c2cnc(NC3CCN(S(=O)(=O)C4CCN(CC5CCN(c6ccc7c(c6)n(C)c(=O)n7C6CCC(=O)NC6=O)CC5)C4)CC3)nc21]

### Step A. 3-{5-[4-(1,3-Dioxolan-2-yl)piperidin-1-yl]-3-methyl-2-oxo-1,3-benzodiazol-1-yl}-1-{[2-(trimethylsilyl)-ethoxy]methyl}piperidine-2,6-dione (140-1)

To a solution of Int-33 (500 mg, 1.067 mmol, 1 equiv) in 1,4-dioxane (5 mL) was added 4-(1,3-dioxolan-2-yl)piperidine (167.81 mg, 1.067 mmol, 1 equiv), RuPhos Palladacycle Gen.4 (89.28 mg, 0.107 mmol, 0.1 equiv), RuPhos (49.81 mg, 0.107 mmol, 0.1 equiv), Cs₂CO₃ (1043.35 mg, 3.201 mmol, 3 equiv) at room temperature. The mixture was stirred for 2 h under argon atmosphere. Desired product could be detected by LCMS. The resulting mixture was concentrated under reduced pressure. The residue was purified by silica gel column chromatography, eluted with DCM/MeOH (30/1) to afford 140-1 (265 mg, 45.58%) as a yellow solid. LCMS (ESI, m/z): [M+H]⁺=545.

### Step B. 1-[1-(2,6-Dioxo-1-{[2-(trimethylsilyl)ethoxy]methyl}piperidin-3-yl)-3-methyl-2-oxo-1,3-benzodiazol-5-yl]piperidine-4-carbaldehyde (140-2)

To a solution of 140-1 (265 mg, 0.486 mmol, 1 equiv) in H₂O (0.6 mL) and 1,4-dioxane (3 mL) was added TsOH (335.09 mg, 1.944 mmol, 4 equiv) at room temperature. The mixture was stirred for 2 h at 60° C. Desired product could be detected by LCMS. The reaction was quenched with Et₃N at room temperature. The resulting mixture was extracted with EA (3×30 mL). The combined organic phase was dried over anhydrous sodium sulfate and concentrated under reduced pressure. The residue was purified by silica gel column chromatography, eluted with PE/EA (1/1) to afford 140-2 (110 mg, 45.16%) as a white solid. LCMS (ESI, m/z): [M+H]⁺=501.

### Step C. 3-(3-Methyl-5-{4-[(3-{4-[7′-(2-methylcyclopentyl)-6′-oxospiro-[cyclopropane-1,5′-pyrrolo[2,3-d]pyrimidin]-2′-ylamino]piperidin-1-ylsulfonyl}pyrrolidin-1-yl)methyl]piperidin-1-yl}-2-oxo-1,3-benzodiazol-1-yl)-1-{[2-(trimethylsilyl)ethoxy]methyl}piperidine-2,6-dione (140-3)

To a solution of 140-2 (120 mg, 0.240 mmol, 1 equiv) in DMF (2 mL) was added 1-2 (113.76 mg, 0.240 mmol, 1 equiv), AcOH (0.1 mL, 1.745 mmol, 7.28 equiv) at room temperature. The mixture was stirred for 30 min at room temperature. Then the STAB (152.39 mg, 0.720 mmol, 3 equiv) and Ti(OEt)₄ (546.73 mg, 2.400 mmol, 10 equiv) were added. The mixture was stirred for 1 h at room temperature. Desired product could be detected by LCMS. The resulting mixture was extracted with EA (3×30 mL) and water. The combined organic phase was dried over anhydrous sodium sulfate and concentrated under reduced pressure. The residue was purified by silica gel column chromatography, eluted with DCM/MeOH (30/1) to afford 140-3 (70 mg, 30.44%) as a white solid. LCMS (ESI, m/z): [M+H]⁺=959.

### Step D. 3-(3-Methyl-5-{4-[(3-{4-[7′-(2-methylcyclopentyl)-6′-oxospiro[cyclopropane-1,5′-pyrrolo[2,3-d]pyrimidin]-2′-ylamino]piperidin-1-ylsulfonyl}pyrrolidin-1-yl)methyl]piperidin-1-yl}-2-oxo-1,3-benzodiazol-1-yl)piperidine-2,6-dione (140)

Compound 140 was prepared from 140-3 following the procedure for the synthesis of compound 135 in example 135. LCMS (ESI, m/z): [M+H]⁺=829.4.

### Example 141: 3-(3-Methyl-4-{[7-(7-{4-[7′-(2-methylcyclopentyl)-6′-oxospiro[cyclopropane-1,5′-pyrrolo[2,3-d]pyrimidin]-2′-ylamino]piperidin-1-ylsulfonyl}-2,7-diazaspiro[4.4]nonan-2-yl)heptyl]oxyl-2-oxo-1,3-benzodiazol-1-yl)piperidine-2,6-dione (141)

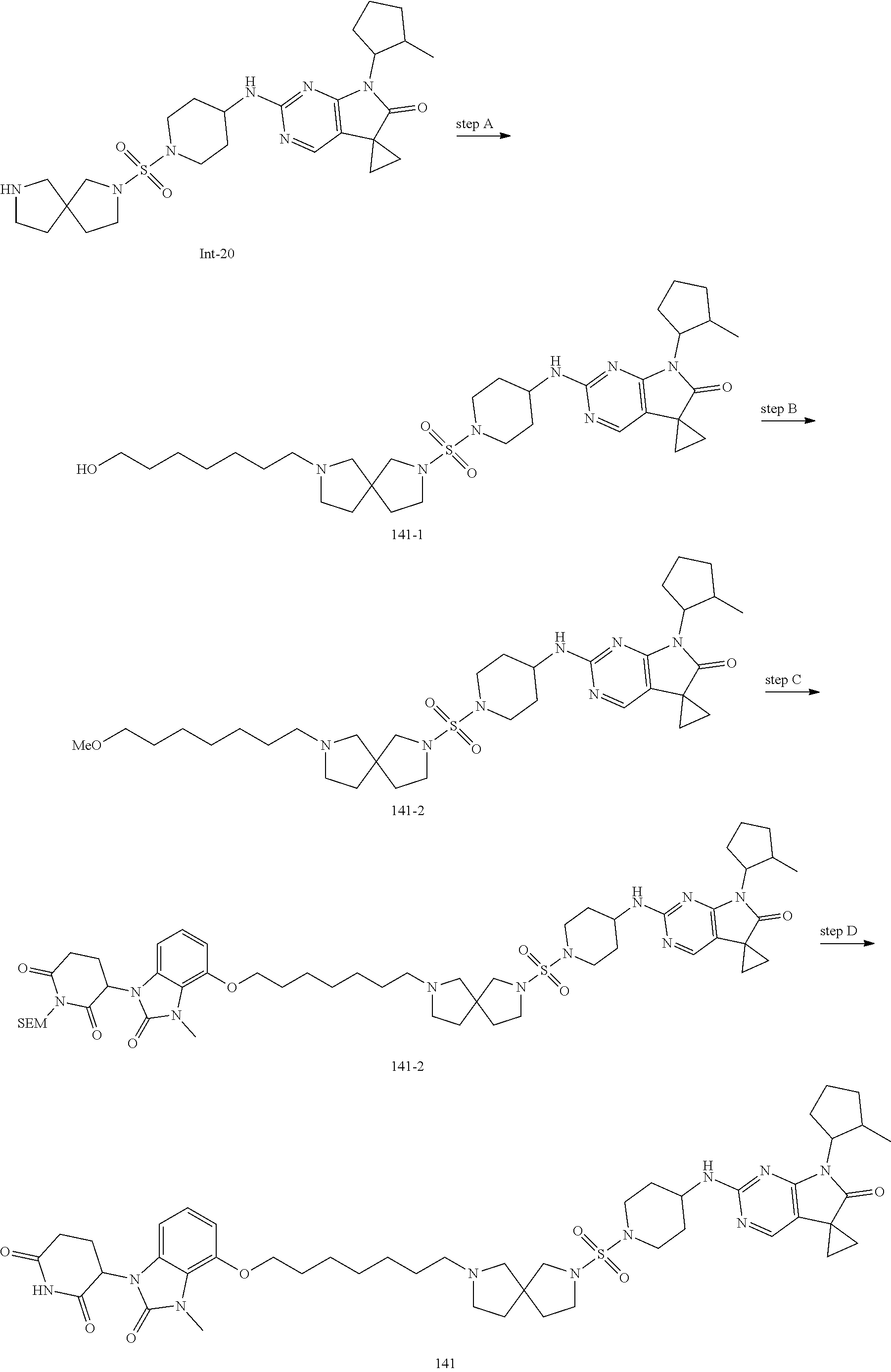 [SMILES: CC1CCCC1N1C(=O)C2(CC2)c2cnc(NC3CCN(S(=O)(=O)N4CCC5(CCNC5)C4)CC3)nc21 ; CC1CCCC1N1C(=O)C2(CC2)c2cnc(NC3CCN(S(=O)(=O)N4CCC5(CCN(CCCCCCCO)C5)C4)CC3)nc21 ; COCCCCCCCN1CCC2(CCN(S(=O)(=O)N3CCC(Nc4ncc5c(n4)N(C4CCCC4C)C(=O)C54CC4)CC3)C2)C1 ; CC1CCCC1N1C(=O)C2(CC2)c2cnc(NC3CCN(S(=O)(=O)N4CCC5(CCN(CCCCCCCOc6cccc7c6n(C)c(=O)n7C6CCC(=O)N(COCC[Si](C)(C)C)C6=O)C5)C4)CC3)nc21 ; CC1CCCC1N1C(=O)C2(CC2)c2cnc(NC3CCN(S(=O)(=O)N4CCC5(CCN(CCCCCCCOc6cccc7c6n(C)c(=O)n7C6CCC(=O)NC6=O)C5)C4)CC3)nc21]

### Step A. 2′-({1-[7-(7-Hydroxyheptyl)-2,7-diazaspiro[4.4]nonan-2-ylsulfonyl]piperi din-4-yl}amino)-7′-(2-methylcyclopentyl)spiro[cyclopropane-1,5′-pyrrolo[2,3-d]pyrimidin]-6′-one (141-1)

A solution of Int-20 (200 mg, 0.378 mmol, 1 equiv), 7-bromoheptan-1-ol (147.33 mg, 0.756 mmol, 2 equiv) in ACN (5 mL) was treated with K₂CO₃ (156.55 mg, 1.134 mmol, 3 equiv) at 25° C. under nitrogen atmosphere followed by the addition of KI (62.68 mg, 0.378 mmol, 1 equiv) at 25° C. The resulting mixture was stirred for 1 h at 80° C. under nitrogen atmosphere. The residue was purified by Prep-TLC (CH₂Cl₂/MeOH 5:1) to afford 141-1 (150 mg, 61.70%) as a white solid. LCMS (ESI, m/z): [M+H]⁺=644.

### Step B. 7-(7-{4-[7′-(2-Methylcyclopentyl)-6′-oxospiro[cyclopropane-1,5′-pyrrolo[2,3-d]pyrimidin]-2′-ylamino]piperidin-1-ylsulfonyl}-2,7-diazaspiro[4.4]nonan-2-yl)heptyl methanesulfonate (141-2)

A solution of 141-1 (140 mg, 0.217 mmol, 1 equiv) in CH₂Cl₂ (5 mL) was treated with Et₃N (0.18 mL, 1.302 mmol, 6 equiv) at 0° C. under nitrogen atmosphere followed by the addition of Methanesulfonic anhydride (113.62 mg, 0.651 mmol, 3 equiv) dropwise at 0° C. The resulting mixture was stirred for 1 h at 0° C. under nitrogen atmosphere. Desired product could be detected by LCMS. The residue was purified by Prep-TLC (CH₂Cl₂/MeOH 5:1) to afford 141-2 (90 mg, 57.33%) as a yellow solid. LCMS (ESI, m/z): [M+H]⁺=722.

### Step C. 3-(3-Methyl-4-{[7-(7-{4-[7′-(2-methylcyclopentyl)-6′-oxospiro[cyclopropane-1,5′-pyrrolo[2,3-d]pyrimidin]-2′-ylamino]piperidin-1-ylsulfonyl}-2,7-diazaspiro[4.4]nonan-2-yl)heptyl]oxy}-2-oxo-1,3-benzodiazol-1-yl)-1-{[2-(trimethylsilyl)ethoxy]methyl}piperidine-2,6-dione (141-3)

A solution of 141-2 (200 mg, 0.277 mmol, 1 equiv), 138-1 (337.01 mg, 0.831 mmol, 3 equiv) in DMSO (2 mL) was treated with K₂CO₃ (114.85 mg, 0.831 mmol, 3 equiv) at 25° C. under nitrogen atmosphere followed by the addition of NaI (41.52 mg, 0.277 mmol, 1 equiv) at 25° C. The resulting mixture was stirred for 16 h at 80° C. under nitrogen atmosphere. Desired product could be detected by LCMS. The residue was purified by reversed-phase flash chromatography with the following conditions: column, C18 silica gel; mobile phase, MeCN in Water (0.1% NH₃·H₂O), 10% to 50% gradient in 10 min; detector, UV 254 nm. to afford 141-3 (130 mg, 45.50%) as a yellow solid. LCMS (ESI, m/z): [M+H]⁺=1031.

### Step D. 3-(3-Methyl-4-{[7-(7-{4-[7′-(2-methylcyclopentyl)-6′-oxospiro[cyclopropane-1,5′-pyrrolo[2,3-d]pyrimidin]-2′-ylamino]piperidin-1-ylsulfonyl}-2,7-diazaspiro[4.4]nonan-2-yl)heptyl]oxy}-2-oxo-1,3-benzodiazol-1-yl)piperidine-2,6-dione (141)

A solution of 141-3 (120 mg, 0.116 mmol, 1 equiv) in CH₂Cl₂ (1 mL) was treated with TFA (875.57 mg, 7.656 mmol, 66 equiv) for 1 min at 0° C. under nitrogen atmosphere. The resulting mixture was stirred for 1 h at 25° C. under nitrogen atmosphere. To the above mixture was added Et₃N (1554.10 mg, 15.312 mmol, 132 equiv), DMEDA (12.31 mg, 0.139 mmol, 1.2 equiv) dropwise over 2 min at 0° C. The resulting mixture was stirred for additional 1 h at 25° C. Desired product could be detected by LCMS. The resulting mixture was concentrated under reduced pressure. The residue was purified by reversed-phase flash chromatography with the following conditions: column, C18 silica gel; mobile phase, MeCN in Water (0.1% NH₃·H₂O), 50% to 60% gradient in 10 min; detector, UV 254 nm. This resulted in compound 141 (41.1 mg, 39.20%) as a white solid. LCMS (ESI, m/z): [M+H]⁺=901.4.

### Example 146: 1-[7-(1-{[3-(4-{7′-[(1S,3S)-3-hydroxycyclohexyl]-6′-oxospiro[cyclopropane-1,5′-pyrrolo[2,3-d]pyrimidin]-2′-ylamino}piperidin-1-ylsulfonyl)phenyl]methyl}piperidin-4-yl)-1-methylindazol-3-yl]-1,3-diazinane-2,4-dione (146)

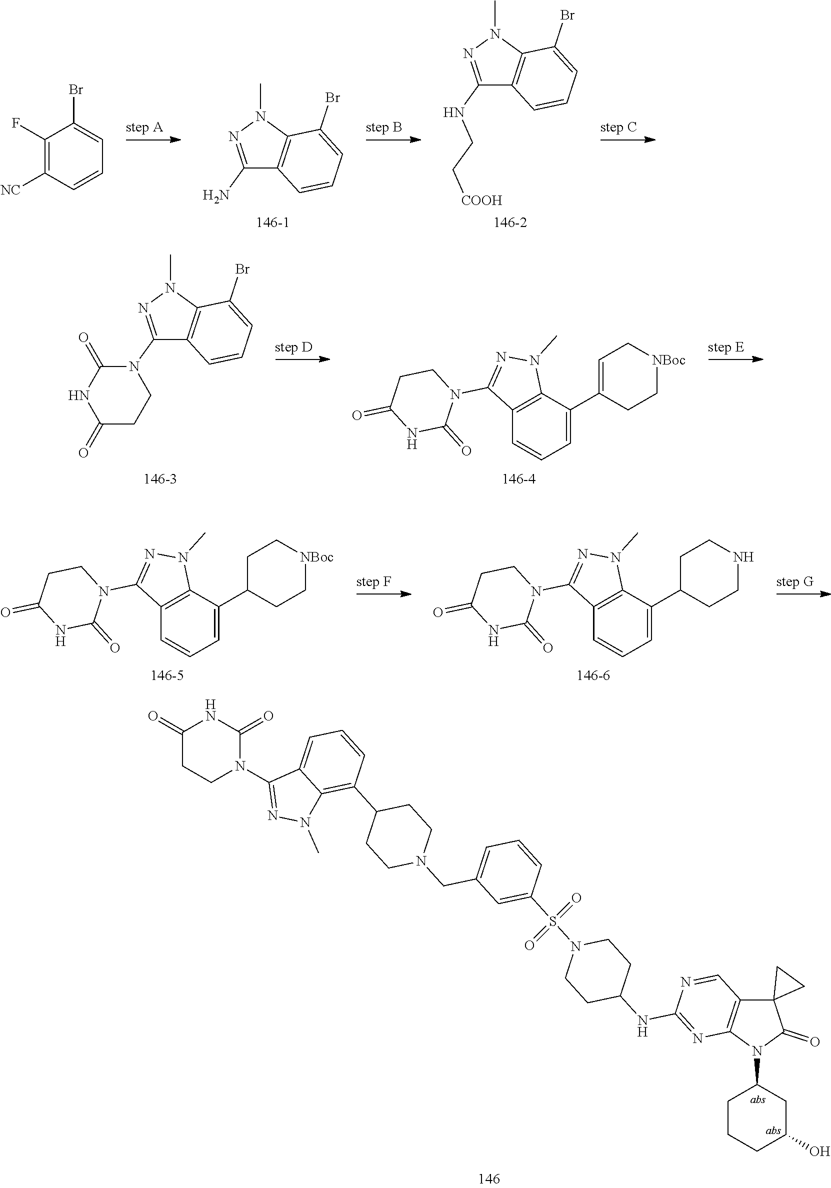

### Step A. 7-Bromo-1-methylindazol-3-amine (146-1)

To a solution of 3-bromo-2-fluorobenzonitrile (28 g, 139.993 mmol, 1 equiv) in EtOH (3000 mL) was added methyl hydrazine (25.80 g, 559.972 mmol, 4 equiv), DIEA (180.94 g, 1399.930 mmol, 10 equiv) at room temperature. The mixture was stirred for overnight at 120° C. Desired product could be detected by LCMS. The resulting mixture was concentrated under reduced pressure. The residue was purified by silica gel column chromatography, eluted with PE/EA (10/1) to afford 146-1 (23 g, 72.67%) as a yellow solid. LCMS (ESI, m/z): [M+H]⁺=226.

### Step B. 3-[(7-Bromo-1-methylindazol-3-yl)amino]propanoic acid (146-2)

To a solution of 146-1 (23 g, 101.735 mmol, 1 equiv) in H₂O (3000 mL) was added acrylic acid (11.00 g, 152.602 mmol, 1.5 equiv), AcOH (18.33 g, 305.205 mmol, 3 equiv) at room temperature. The mixture was stirred for overnight at 100° C. Desired product could be detected by LCMS. The product was precipitated by the addition of water. The residue was purified by trituration with water (5000 mL) to afford 146-2 (18 g, 59.34%) as yellow solid. LCMS (ESI, m/z): [M+H]⁺=298.

### Step C. 1-(7-Bromo-1-methylindazol-3-yl)-1,3-diazinane-2,4-dione (146-3)

To a solution of 146-2 (15 g, 50.312 mmol, 1 equiv) in AcOH (2000 mL) was added urea (15.11 g, 251.560 mmol, 5 equiv) at room temperature. The mixture was stirred for overnight at 120° C. Desired product could be detected by LCMS. The resulting mixture was concentrated under reduced pressure. The residue was purified by silica gel column chromatography, eluted with PE/EA (3/1) to afford 146-3 (3.5 g, 21.53%) as a yellow solid. LCMS (ESI, m/z): [M+H]⁺=323.

### Step D. Tert-butyl 4-[3-(2,4-dioxo-1,3-diazinan-1-yl)-1-methylindazol-7-yl]-3,6-dihydro-2H-pyridine-1-carboxylate (146-4)

To a solution of 146-3 (2 g, 6.189 mmol, 1 equiv) in 1,4-dioxane (20 mL)/H₂O (2 mL) was added tert-butyl 4-(4,4,5,5-tetramethyl-1,3,2-dioxaborolan-2-yl)-3,6-dihydro-2H-pyridine-1-carboxylate (3.83 g, 12.378 mmol, 2 equiv), XPhos Pd G3 (0.79 g, 0.928 mmol, 0.15 equiv), K₃PO₄ (3.94 g, 18.567 mmol, 3 equiv) at room temperature. The mixture was stirred for 1 hour 80° C. Desired product could be detected by LCMS. The resulting mixture was concentrated under reduced pressure. The residue was purified by silica gel column chromatography, eluted with DCM/MeOH (30/1) to afford 146-4 (2.5 g, 94.93%) as a white solid. LCMS (ESI, m/z): [M+H]⁺=426.

### Step E. Tert-butyl 4-[3-(2,4-dioxo-1,3-diazinan-1-yl)-1-methylindazol-7-yl]piperi dine-1-carboxylate (146-5)

To a solution of 146-4 (500 mg, 1.175 mmol, 1 equiv) in CF₃CH₂OH (5 mL) was added Pd/C (250 mg) at room temperature. The mixture was stirred for 3 hours under hydrogen atmosphere using a hydrogen balloon, filtered through a Celite pad and concentrated under reduced pressure to afford 146-5 (430 mg, 85.59%) as white solid. LCMS (ESI, m/z): [M+H]⁺=428.

### Step F. 1-[1-Methyl-7-(piperidin-4-yl) indazol-3-yl]-1,3-diazinane-2,4-dione (146-6)

To a stirred solution of 146-5 (200 mg, 0.468 mmol, 1 equiv) in DCM (5 mL) was added 4M HCl (gas) in 1,4-dioxane (1 mL) at 0 degrees C. The resulting mixture was stirred for 1 h at 25 degrees C. Desired product could be detected by LCMS. The resulting mixture was diluted with NaHCO₃ (aq. 5 mL) and extracted with EtOAc (3×10 mL). The combined organic layers were dried over anhydrous Na₂SO₄ and concentrated under reduced pressure. The residue was purified by reversed-phase Combi-Flash, eluted with CH₃CN:H₂O (0.5% NH₄HCO₃)=5%-60%. This resulted in 146-6 (130 mg, 76.39%) as a white solid. LCMS (ESI, m/z): [M+H]⁺=328.

### Step G. 1-[7-(1-{[3-(4-{7′-[(1S,3S)-3-Hydroxycyclohexyl]-6′-oxospiro[cyclopropane-1,5′-pyrrolo[2,3-d]pyrimidin]-2′-ylamino}piperidin-1-ylsulfonyl)phenyl]methyl}piperidin-4-yl)-1-methylindazol-3-yl]-1,3-diazinane-2,4-dione (146)

To a stirred mixture of 146-6 (80 mg, 0.244 mmol, 1 equiv) and Int-6 (144.30 mg, 0.244 mmol, 1 equiv) and KI (8.11 mg, 0.049 mmol, 0.2 equiv) in MeCN (5 mL) were added K₂CO₃ (101.31 mg, 0.732 mmol, 3 equiv) at 25 degrees C. under N₂. The resulting mixture was stirred for 1 h at 80 degrees C. Desired product could be detected by LCMS. The resulting mixture was diluted with water (5 mL) and extracted with EtOAc (3×10 mL). The combined organic layers were dried over anhydrous Na₂SO₄ and concentrated under reduced pressure. The residue was purified by reversed-phase Combi-Flash, eluted with CH₃CN:H₂O (0.5% NH₄HCO₃)=5%-60%. This resulted in compound 146 (31.0 mg, 14.58%) as a white solid. LCMS (ESI, m/z): [M+H]⁺=837.4.

### Example 147: 3-[5-(1-{[3-(4-{7′-[(1R,3R)-3-Hydroxycyclohexyl]-6′-oxospiro[cyclopropane-1,5′-pyrrolo[2,3-d]pyrimidin]-2′-ylamino}piperidin-1-ylsulfonyl)phenyl]methyl}piperidin-4-yl)-3-methyl-2-oxo-1,3-benzodiazol-1-yl]piperidine-2,6-dione (147)

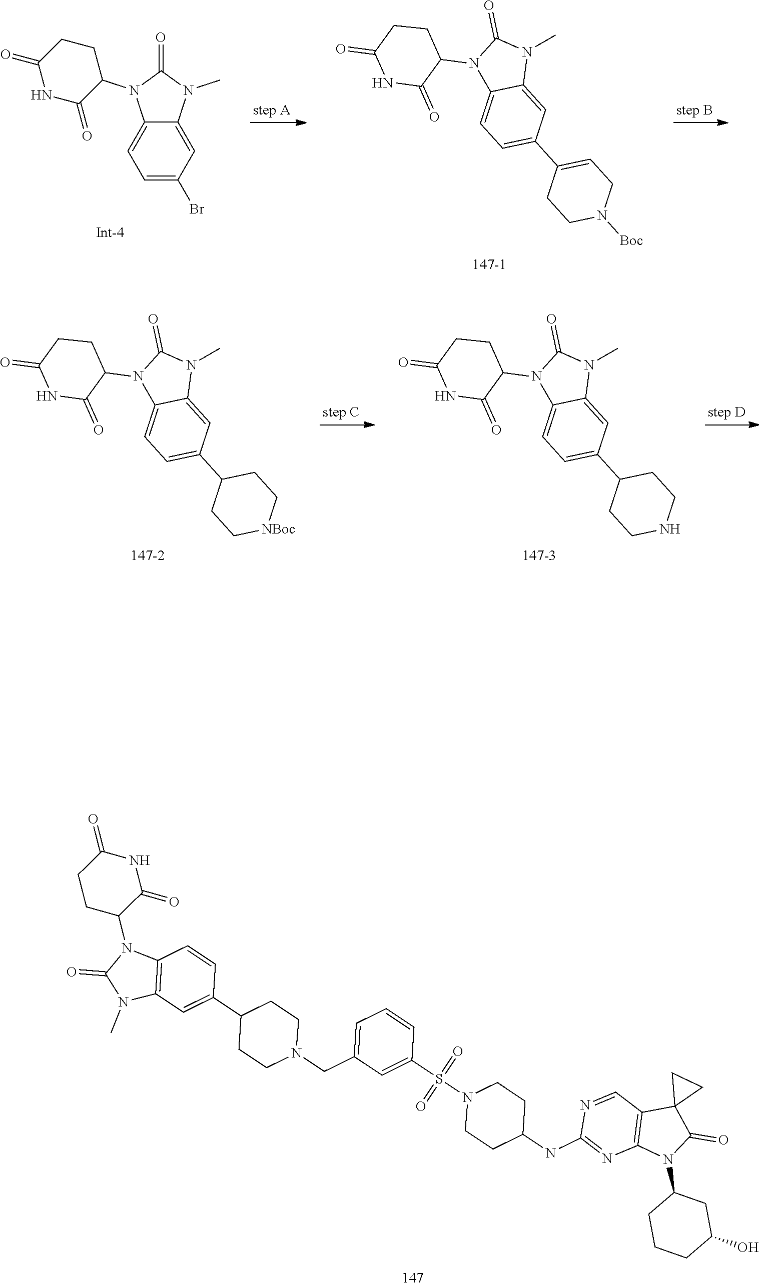 [SMILES: Cn1c(=O)n(C2CCC(=O)NC2=O)c2ccc(Br)cc21 ; Cn1c(=O)n(C2CCC(=O)NC2=O)c2ccc(C3=CCN(C(=O)OC(C)(C)C)CC3)cc21 ; Cn1c(=O)n(C2CCC(=O)NC2=O)c2ccc(C3CCN(C(=O)OC(C)(C)C)CC3)cc21 ; Cn1c(=O)n(C2CCC(=O)NC2=O)c2ccc(C3CCNCC3)cc21 ; Cn1c(=O)n(C2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)c4)CC3)cc21]

### Step A. 7-Bromo-1-methylindazol-3-amine (146-1)

A solution of Int 4 (3 g, 8.872 mmol, 1 equiv), tert-butyl 4-(4,4,5,5-tetramethyl-1,3,2-dioxaborolan-2-yl)-3,6-dihydro-2H-pyridine-1-carboxylate (3.02 g, 9.759 mmol, 1.1 equiv), Pd(DtBPF)Cl₂ (0.58 g, 0.887 mmol, 0.1 equiv) and K₂CO₃ (2.45 g, 17.744 mmol, 2 equiv) in dioxane (10 mL) was stirred for 1 h at 80° C. under argon atmosphere. Desired product could be detected by LCMS. The resulting mixture was concentrated under reduced pressure. The residue was purified by silica gel column chromatography, eluted with DCM/MeOH (20:1) to afford 146-1 (2.8 g, 71.65%) as a off-white solid. LCMS (ESI, m/z): [M+H]⁺=441.

Compound 147-3 was prepared from 147-1 following the procedure for the synthesis of compound 13-3 in example 13. Compound 147 was prepared from 147-3 following the procedure for the synthesis of compound 146 in example 146.

### Example 149: 3-(5-{4-[3-(4-{7′-[(1R,3R)-3-Hydroxycyclohexyl]-6′-oxospiro[cyclopropane-1,5′-pyrrolo[2,3-d]pyrimidin]-2′-ylamino}piperidin-1-ylsulfonyl)phenoxy]phenyl}-3-methyl-2-oxo-1,3-benzodiazol-1-yl)piperidine-2,6-dione (149)

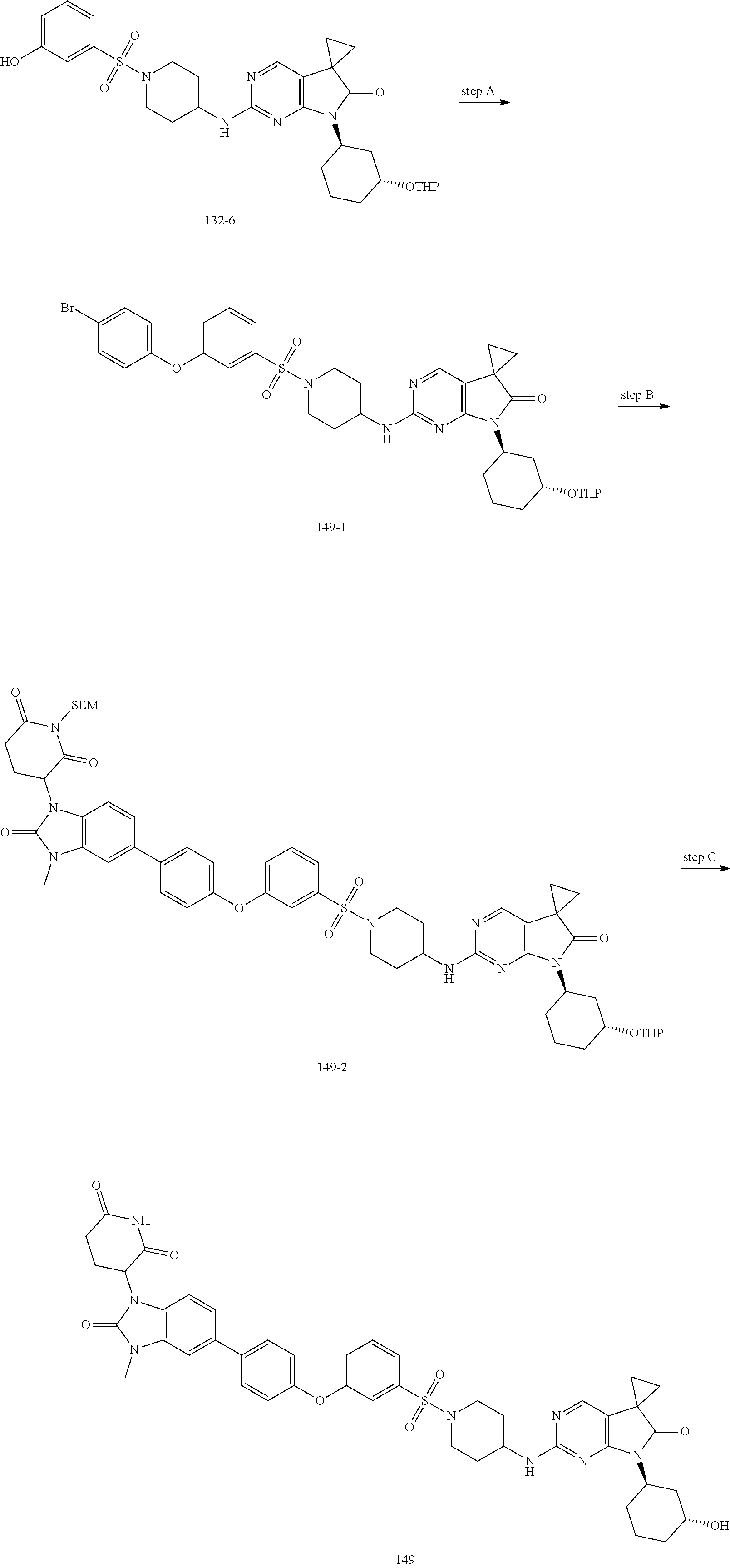

### Step A. 2′-({1-[3-(4-Bromophenoxy)benzenesulfonyl]piperidin-4-yl}amino)-7′-[(1 R,3R)-3-(oxan-2-yloxy)cyclohexyl]spiro[cyclopropane-1,5′-pyrrolo[2,3-d]pyrimidin]-6′-one (149-1)

To a stirred solution of 132-6 (400 mg, 0.669 mmol, 1 equiv), 4-bromophenylboric acid (403.19 mg, 2.007 mmol, 3 equiv) and Cu(OAc)₂ (243.10 mg, 1.338 mmol, 2 equiv) in DCM (20 mL) were added Pyridine (105.87 mg, 1.338 mmol, 2 equiv) and TEA (270.87 mg, 2.676 mmol, 4 equiv) dropwise at room temperature under nitrogen atmosphere. The resulting mixture was stirred for 16 h at room temperature under air atmosphere. Desired product could be detected by LCMS. The residue was purified by Prep-TLC (CH₂Cl₂/MeOH 20:1) to afford 149-1 (220 mg, 43.67%) as a yellow solid. LCMS (ESI, m/z): [M+H]⁺=752.

### Step B. 3-(3-Methyl-5-{4-[3-(4-{7′-[(1R,3R)-3-(oxan-2-yloxy)cyclohexyl]-6′-oxospiro[cyclopropane-1,5′-pyrrolo[2,3-d]pyrimidin]-2′-ylamino}piperidin-1-ylsulfonyl)phenoxy]phenyl}-2-oxo-1,3-benzodiazol-1-yl)-1-{[2-(trimethylsilyl)ethoxy]methyl}piperidine-2,6-dione (149-2)

To a stirred solution of 149-1 (100 mg, 0.133 mmol, 1 equiv) and 3-(3-methyl-2-oxo-5-(4,4,5,5-tetramethyl-1,3,2-dioxaborolan-2-yl)-2,3-dihydro-1H-benzo[d]imidazol-1-yl)-1-((2-(trimethylsilyl)ethoxy)methyl)piperidine-2,6-dione (75.33 mg, 0.146 mmol, 1.1 equiv) in dioxane (4 mL)/H₂O (0.8 mL) were added Pd(DtBPF)Cl₂ (17.32 mg, 0.027 mmol, 0.2 equiv) and NaOAc (32.69 mg, 0.399 mmol, 3 equiv) at room temperature under nitrogen atmosphere. The resulting mixture was stirred for 15 min at 60° C. under nitrogen atmosphere. Desired product could be detected by LCMS. The residue was purified by Prep-TLC (PE/EA 1:1) to afford 149-2 (50 mg, 35.46%) as a white solid. LCMS (ESI, m/z): [M+H]⁺=1061.

### Step C. 3-(5-{4-[3-(4-{7′-[(1R,3R)-3-Hydroxycyclohexyl]-6′-oxospiro[cyclopropane-1,5′-pyrrolo[2,3-d]pyrimidin]-2′-ylamino}piperidin-1-ylsulfonyl)phenoxy]phenyl}-3-m ethyl-2-oxo-1,3-benzodiazol-1-yl)piperidine-2,6-dione (149)

A solution of 149-2 (50 mg, 0.047 mmol, 1 equiv) and TFA (1 mL) in DCM (3 mL) was stirred for 1 h at 0° C. under nitrogen atmosphere. Desired product could be detected by LCMS. The mixture was neutralized to pH 8 with saturated NaHCO₃ (aq.). The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. This resulted in compound 149 (31.5 mg, 78.47%) as a white solid. LCMS (ESI, m/z): [M+H]⁺=847.3.

### Example 151&152: 1-(6-{1-[(1R)-1-[3-(4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl)phenyl]ethyl]piperidin-4-yl}-1-methylindazol-3-yl)-1,3-diazinane-2,4-dione (151) & 1-(6-{1-[(1S)-1-[3-(4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl)phenyl]ethyl]piperidin-4-yl}-1-methylindazol-3-yl)-1,3-diazinane-2,4-dione (152)

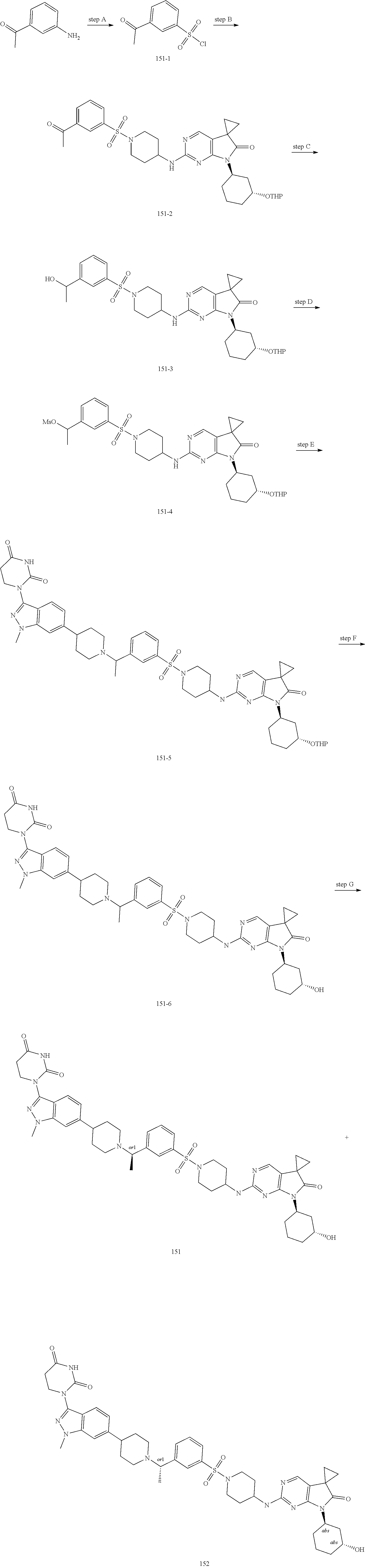

### Step A. 3-Acetylbenzenesulfonyl chloride (151-1)

To a stirred mixture of M-aminoacetophenone (2 g, 14.797 mmol, 1 equiv) in concentrated HCl (30.00 mL, 360.011 mmol, 24.33 equiv) was added NaNO₂ (2.04 g, 29.594 mmol, 2 equiv) and H₂O (10 mL) at 0° C. The resulting mixture was stirred for 0.5 h at 0° C. under air atmosphere. To the above mixture was added NaHSO₃ (15.40 g, 147.970 mmol, 10 equiv) and CuSO₄ (0.24 g, 1.480 mmol, 0.1 equiv) at 0° C. The resulting mixture was stirred for additional 1 h at 0° C. The reaction was quenched by the addition of water (10 mL). The resulting mixture was extracted with EA. The combined organic layers were dried over anhydrous Na₂SO₄. After filtration, the filtrate was concentrated under reduced pressure. The residue was purified by silica gel column chromatography, eluted with PE/EA (3:1) to afford 151-1 (1.5 g, 41.17%) as a brown oil.

### Step B. 2′-{[1-(3-Acetylbenzenesulfonyl)piperidin-4-yl]amino}-7′-[(1R,3R)-3-(oxan-2-yloxy)cyclohexyl]spiro[cyclopropane-1,5′-pyrrolo[2,3-d]pyrimidin]-6′-one (151-2)

To a stirred mixture of 7b (780 mg, 1.766 mmol, 1.00 equiv) and TEA (536.24 mg, 5.298 mmol, 3 equiv) in DCM (8 mL) was added 151-1 (4055.35 mg, 18.543 mmol, 10.50 equiv) at 25° C. The resulting mixture was stirred for 1 h at 40° C. Desired product could be detected by LCMS. The resulting mixture was concentrated under reduced pressure. The residue was purified by silica gel column chromatography, eluted with MeOH/DCM (1:20) to afford 151-2 (829 mg, 70.12%) as a white solid. LCMS (ESI, m/z): [M+H]⁺=624.

### Step C. 2′-({1-[3-(1-Hydroxyethyl)benzenesulfonyl]piperidin-4-yl}amino)-7′-[(1R,3R)-3-(oxan-2-yloxy)cyclohexyl]spiro[cyclopropane-1,5′-pyrrolo[2,3-d]pyrimidin]-6′-one (151-3)

To a stirred mixture of 151-2 (800 mg, 1.283 mmol, 1 equiv) in EtOH (8 mL) was added NaBH₄ (58.22 mg, 1.540 mmol, 1.2 equiv) at 0° C. The resulting mixture was stirred for 1 h at 0° C. Desired product could be detected by LCMS. The reaction was quenched by the addition of water. The resulting mixture was extracted with EtOAc. The combined organic layers were dried over anhydrous Na₂SO₄. After filtration, the filtrate was concentrated under reduced pressure. The residue was purified by silica gel column chromatography, eluted with DCM/MeOH (20:1) to afford 151-3 (700 mg, 81.11%) as a white solid. LCMS (ESI, m/z): [M+H]⁺=626.

### Step D. 1-[3-(4-{7′-[(1R,3R)-3-(Oxan-2-yloxy)cyclohexyl]-6′-oxospiro[cyclopropane-1,5′-pyrrolo[2,3-d]pyrimidin]-2′-ylamino}piperidin-1-ylsulfonyl)phenyl]ethyl methanesulfonate (151-4)

To a stirred mixture of 151-3 (600 mg, 0.959 mmol, 1 equiv) and TEA (388.09 mg, 3.836 mmol, 4 equiv) in DCM (6 mL) was added MsCl (219.64 mg, 1.918 mmol, 2 equiv) dropwise at 0° C. The resulting mixture was stirred for 1 h at 0° C. Desired product could be detected by LCMS. The reaction was quenched by the addition of NaHCO₃ (10 mL). The resulting mixture was extracted with DCM. The combined organic layers were dried over anhydrous Na₂SO₄. After filtration, the filtrate was concentrated under reduced pressure. The residue was purified by silica gel column chromatography, eluted with DCM/MeOH (20:1) to afford 151-4 (500 mg, 72.68%) as a white solid. LCMS (ESI, m/z): [M+H]⁺=704.

### Step E. 1-[1-Methyl-6-(1-{1-[3-(4-{7′-[(1R,3R)-3-(oxan-2-yloxy)cyclohexyl]-6′-oxo spiro[cyclopropane-1,5′-pyrrolo[2,3-d]pyrimidin]-2′-ylamino}piperidin-1-ylsulfonyl)phenyl]ethyl}piperidin-4-yl) indazol-3-yl]-1,3-diazinane-2,4-dione (151-5)

To a stirred mixture of 151-4 (90 mg, 0.128 mmol, 1 equiv) and Int 24 (41.86 mg, 0.128 mmol, 1 equiv) in ACN (1.5 mL) was added K₂CO₃ (106.03 mg, 0.768 mmol, 6 equiv) and KI (2.12 mg, 0.013 mmol, 0.1 equiv). The resulting mixture was stirred for 1 h at 60° C. Desired product could be detected by LCMS. The resulting mixture was concentrated under reduced pressure. The residue was purified by silica gel column chromatography, eluted with MeOH/DCM (1:20) to afford 151-5 (70 mg, 58.48%) as a white solid. LCMS (ESI, m/z): [M+H]⁺=935.

### Step F. 1-[6-(1-{1-[3-(4-{7′-[(1R,3R)-3-Hydroxycyclohexyl]-6′-oxospiro[cyclopropane-1,5′-pyrrolo[2,3-d]pyrimidin]-2′-ylamino}piperidin-1-ylsulfonyl)phenyl]ethyl}piperidin-4-yl)-1-methylindazol-3-yl]-1,3-diazinane-2,4-dione (151-6)

To a stirred solution of 151-5 (85 mg, 0.091 mmol, 1 equiv) in ACN (4.25 mL) was added 4M HCl (gas) in 1,4-dioxane (0.85 mL) dropwise at 0° C. The resulting mixture was stirred for additional 1 h at 0° C. Desired product could be detected by LCMS. The resulting mixture was concentrated under reduced pressure. The resulting mixture was diluted with DCM and was neutralized with Et₃N. The residue was purified by reversed-phase Combi-Flash, eluted with CH₃CN:H₂O (0.5% NH₄HCO₃)=40%-50%. This resulted in 151-6 (50 mg, 46.73%) as a white solid. LCMS (ESI, m/z): [M+H]⁺=851.

### Step G. 1-(6-{1-[(1R)-1-[3-(4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl)phenyl]ethyl]piperidin-4-yl}-1-methylindazol-3-yl)-1,3-diazinane-2,4-dione (151) & 1-(6-{1-[(1S)-1-[3-(4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl)phenyl]ethyl]piperidin-4-yl}-1-methylindazol-3-yl)-1,3-diazinane-2,4-dione (152)

The 151-6 (50 mg) was separated by Chiral-HPLC, Column: CHIRAL ART Amylose-C Neo, Mobile Phase: HEX (10 mM NH₃):IPA=50:50, to afford compound 151 (15.5 mg, 30.75%) and compound 152 (14.7 mg, 28.84%) as a white solid. LCMS (ESI, m/z): [M+H]⁺=851.4.

### Example 153: 1-{6-[1-({3-[(3R,4S)-4-{7′-[(1R,3R)-3-Hydroxycyclohexyl]-6′-oxospiro[cyclopropane-1,5′-pyrrolo[2,3-d]pyrimidin]-2′-ylamino}-3-methylpiperidin-1-ylsulfonyl]phenyl}methyl)piperidin-4-yl]-1-methylindazol-3-yl}-1,3-diazinane-2,4-dione (153)

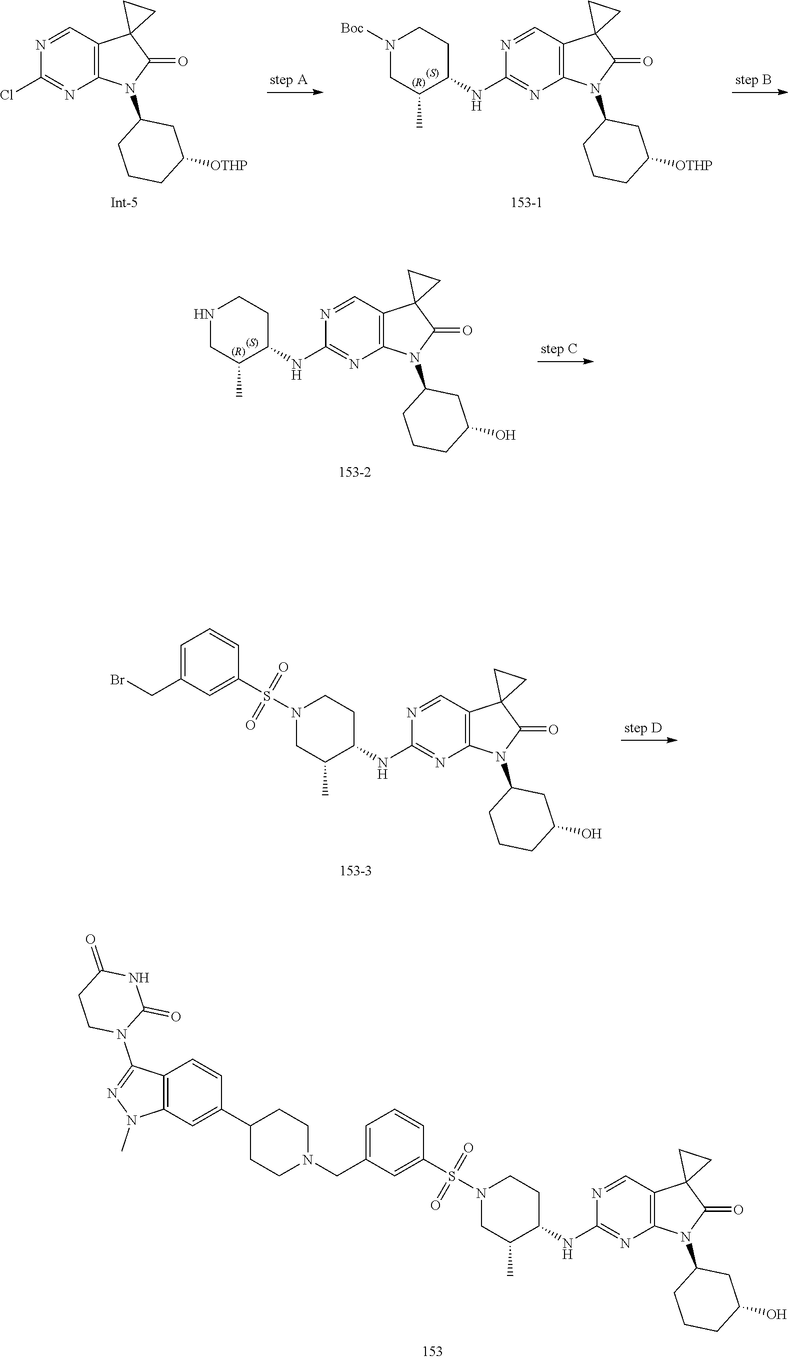

### Step A. Tert-butyl (3R,4S)-3-methyl-4-{7′-[(1R,3R)-3-(oxan-2-yloxy)cyclohexyl]-6′-oxospiro[cyclopropane-1,5′-pyrrolo[2,3-d]pyrimidin]-2′-ylamino}piperidine-1-carboxylate (153-1)

To a stirred mixture of Int-5 and tert-butyl (3R,4S)-4-amino-3-methylpiperidine-1-carboxylate (340.29 mg, 1.588 mmol, 1.2 equiv) in dioxane (10 mL) were added Ruphos (61.75 mg, 0.132 mmol, 0.1 equiv), Cs₂CO₃ (1293.38 mg, 3.969 mmol, 3 equiv) and Pd₂(dba)₃ (121.17 mg, 0.132 mmol, 0.1 equiv) at room temperature under argon atmosphere. The resulting mixture was stirred for overnight at 100° C. under argon atmosphere. The reaction was monitored by LCMS. The resulting mixture was concentrated under reduced pressure. The residue was purified by silica gel column chromatography, eluted with PE/EA (1:1) to afford 153-1 (200 mg, 27.20%) as a white solid. LCMS (ESI, m/z): [M+H]⁺=556.3

### Step B. 7′-((1R,3R)-3-Hydroxycyclohexyl)-2′-(((3R,4S)-3-methylpiperidin-4-yl)amino)spiro[cyclopropane-1,5′-pyrrolo[2,3-d]pyrimidin]-6′(7′H)-one (153-2)

A mixture of 153-1 (200 mg, 0.360 mmol, 1 equiv) and 4M HCl in dioxane (2 mL) in ACN (2 mL) was stirred for 1 h at 0° C. The reaction was monitored by LCMS. The resulting mixture was concentrated under reduced pressure. The crude product was used in the next step directly without further purification. LCMS (ESI, m/z): [M+H]⁺=372.

### Step C. 2′-(((3R,4S)-1-((3-(Bromomethyl)phenyl)sulfonyl)-3-methylpiperidin-4-yl)amino)-7′-((1R,3R)-3-hydroxycyclohexyl)spiro[cyclopropane-1,5′-pyrrolo[2,3-d]pyrimidin]-6′(7′H)-one (153-3)

To a stirred mixture of 153-2 (200 mg, 0.538 mmol, 1 equiv) and DIEA (347.92 mg, 2.690 mmol, 5 equiv) in DCM (2 mL) was added 3-(bromomethyl)benzenesulfonyl chloride (159.63 mg, 0.592 mmol, 1.1 equiv) at 0° C. The resulting mixture was stirred for 30 min at 0° C. The reaction was monitored by LCMS. The resulting mixture was concentrated under reduced pressure. The crude product was used in the next step directly without further purification. LCMS (ESI, m/z): [M+H]⁺=604.

### Step D. 1-{6-[1-({3-[(3R,4S)-4-{7′-[(1R,3R)-3-Hydroxycyclohexyl]-6′-oxospiro[cyclopropane-1,5′-pyrrolo[2,3-d]pyrimidin]-2′-ylamino}-3-methylpiperidin-1-ylsulfonyl]phenyl}methyl)piperidin-4-yl]-1-methylindazol-3-yl}-1,3-diazinane-2,4-dione (153)

To a stirred mixture of 153-3 (100 mg, 0.165 mmol, 1 equiv) and Int-24 (72.22 mg, 0.198 mmol, 1.2 equiv) in DMF (2 mL) were added K₂CO₃ (137.16 mg, 0.990 mmol, 6 equiv) and KI (13.73 mg, 0.083 mmol, 0.5 equiv) at room temperature. The resulting mixture was stirred for 1 h at 60° C. The reaction was monitored by LCMS. The residue was purified by reversed-phase flash chromatography with the following conditions: column, C18 silica gel; mobile phase, MeCN in Water (0.1% NH₃·H₂O+10 mmol/L NH₄HCO₃), 10% to 50% gradient in 10 min; detector, UV 254 nm. This resulted in compound 153 (13 mg, 8.95%) as a white solid. LCMS (ESI, m/z): [M+H]⁺=851.3.

### Example 155: 1-{6-[1-({3-[(3S,4R)-3-Fluoro-4-{7′-[(1R,3R)-3-hydroxycyclohexyl]-6′-oxospiro[cyclopropane-1,5′-pyrrolo[2,3-d] pyrimidin]-2′-ylamino} piperidin-1-ylsulfonyl] phenyl} methyl) piperidin-4-yl]-1-methylindazol-3-yl}-1,3-diazinane-2,4-dione (155)

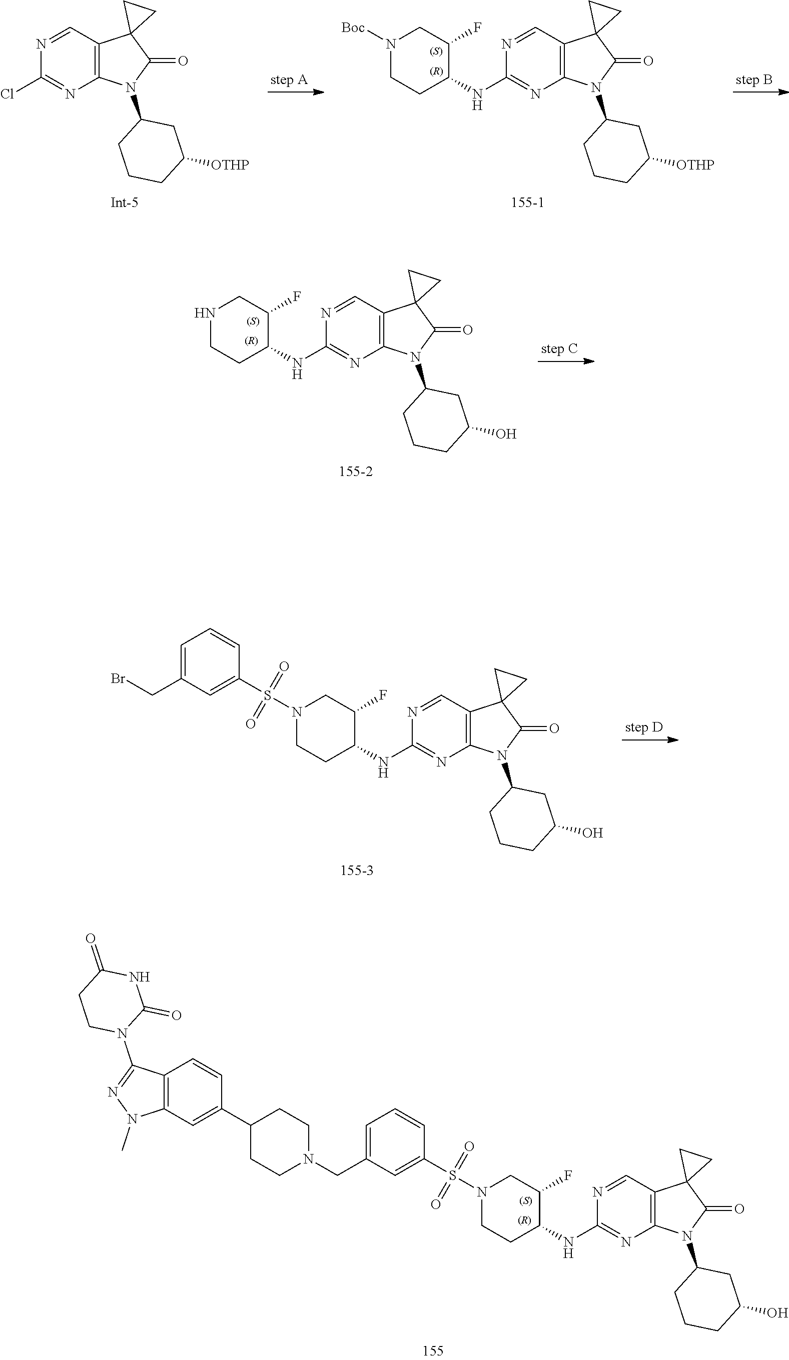

### Step A. Tert-butyl-tert-butyl-(3S,4R)-3-fluoro-4-{7′-[(1R,3R)-3-(oxan-2-yloxy)-cyclohexyl]-6′-oxospiro [cyclopropane-1,5′-pyrrolo[2,3-d]pyrimidin]-2′-ylamino}piperidine-1-carboxylate (155-1)

To a solution of Int-5 (300 mg, 0.794 mmol, 1 equiv) and tert-butyl (3S,4R)-4-amino-3-fluoropiperidine-1-carboxylate (207.95 mg, 0.953 mmol, 1.2 equiv) in 1,2-dioxane (5 mL) were added RuPhos (74.10 mg, 0.159 mmol, 0.2 equiv), RuPhos Palladacycle Gen.3 (134.97 mg, 0.159 mmol, 0.2 equiv) and sodium 2-methylpropan-2-olate (776.03 mg, 2.382 mmol, 3 equiv) at room temperature under nitrogen atmosphere. The resulting mixture was stirred for 2 h at 90° C. under nitrogen atmosphere. Desired product could be detected by LCMS. The resulting mixture was diluted with water and extracted with EtOAc (3×15 mL). The combined organic layers were washed with brine, dried over anhydrous Na₂SO₄. The resulting mixture was concentrated under reduced pressure. The residue was purified by silica gel column chromatography, eluted with PE/EA (1:3) to afford 155-1 (100 mg, 22.51%) as a yellow solid. LCMS (ESI, m/z): [M+H]⁺=560

### Step B-D. 1-{6-[1-({3-[(3S,4R)-3-Fluoro-4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl]phenyl}methyl) piperidin-4-yl]-1-methylindazol-3-yl}-1,3-diazinane-2,4-dione (155)

Compound 155 was prepared from 155-1 following the procedure for the synthesis of compound 153 in example 153. LCMS (ESI, m/z): [M+H]⁺=855.3.

### Example 159: 1-[6-(1-{[3-(4-{7′-[(1R,3R)-3-Hydroxycyclohexyl]-6′-oxospiro [cyclopropane-1,5′-pyrrolo[2,3-d] pyrimidin]-2′-ylamino} piperidin-1-ylsulfonyl)-2-methylphenyl] methyl} piperidin-4-yl)-1-methylindazol-3-yl]-1,3-diazinane-2,4-dione (159)

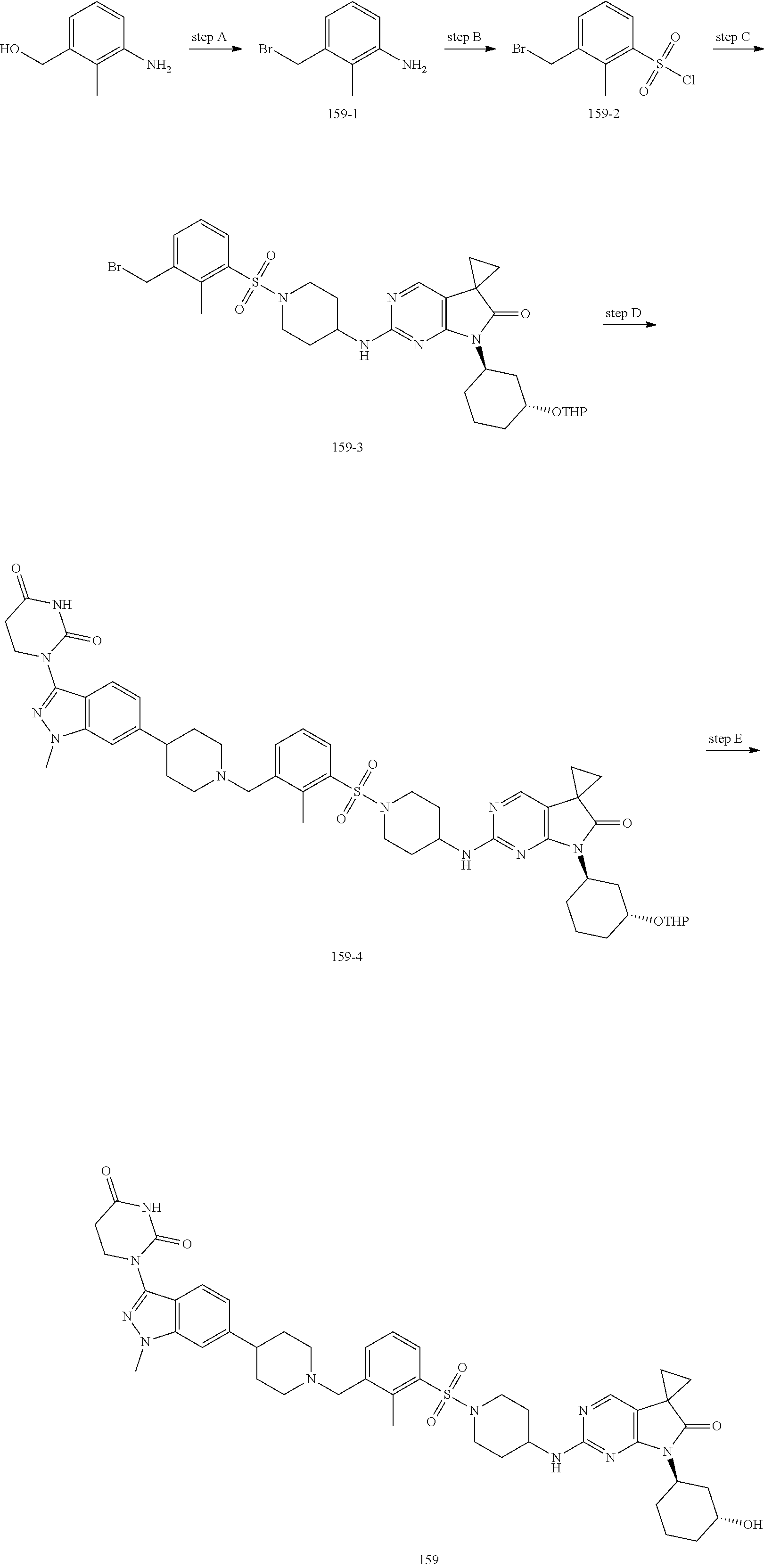

### Step A. 3-(Bromomethyl)-2-methylaniline (159-1)

To a stirred solution of (3-amino-2-methylphenyl) methanol (5 g, 36.448 mmol, 1 equiv) in HBr in water (100.00 mL) at room temperature under nitrogen atmosphere. The resulting mixture was stirred for overnight at room temperature under nitrogen atmosphere. Desired product could be detected by LCMS. The resulting mixture was concentrated under reduced pressure. The product was precipitated by the addition of EtOAc. This resulted in 159-1 (6.1 g, 83.65%) as a yellow solid. LCMS (ESI, m/z): [M+H]⁺=200.

### Step B. 3-(Bromomethyl)-2-methylbenzenesulfonyl chloride (159-2)

To a solution of 159-1 (3 g, 14.994 mmol, 1 equiv) and concentrated HCl (3 mL) in H₂O (12 mL) was added NaNO₂ (155.18 mg, 2.249 mmol, 0.15 equiv) in H₂O (8 mL) was stirred at 0° C. for 30 min under nitrogen atmosphere. To the above mixture was added CuCl₂ (302.39 mg, 2.249 mmol, 0.15 equiv) and SOCl₂ (8.00 mL, 110.356 mmol, 7.36 equiv) in H₂O (50 mL). The resulting mixture was stirred for additional 1 h at 0° C. Desired product could be detected by LCMS. The resulting mixture was concentrated under reduced pressure to afford 159-2 (1.2 g, 28.22%) as a crude product light yellow oil. The crude product was used directly for next step.

### Step C. 2′-({1-[3-(Bromomethyl)-2-methylbenzenesulfonyl]piperidin-4-yl}amino)-7′-[(1R,3R)-3-(oxan-2-yloxy)cyclohexyl]spiro[cyclopropane-1,5′-pyrrolo[2,3-d]pyrimidin]-6′-one (159-3)

To a solution of 7b (151.49 mg, 0.353 mmol, 1 equiv) and DIEA (0.5 mL) in DCM (3 mL) was added 159-2 (100 mg, 0.353 mmol, 1 equiv) at 0° C. The resulting mixture was stirred for 1 h at 0° C. under nitrogen atmosphere. Desired product could be detected by LCMS. The mixture was diluted with water. The resulting mixture was extracted with DCM (3×100 mL). The combined organic layers were washed with brine, dried over anhydrous Na₂SO₄. After filtration, the filtrate was concentrated under reduced pressure. The resulting mixture was concentrated under reduced pressure. The residue was purified by silica gel column chromatography, eluted with PE/EA (3:1) to afford 159-3 (50 mg, 20.59%) as a white solid. LCMS (ESI, m/z): [M+H]⁺=688.

### Step D. 1-[1-Methyl-6-(1-{1-[2-methyl-3-(4-{7′-[(1R,3R)-3-(oxan-2-yloxy) cyclohexyl]-6′-oxospiro [cyclopropane-1,5′-pyrrolo [2,3-d]pyrimidin]-2′-ylamino}piperidin-1-ylsulfonyl) phenyl]methyl}piperidin-4-yl) indazol-3-yl]-1,3-diazinane-2,4-dione (159-4)

To a stirred solution of Int-24 (28.52 mg, 0.088 mmol, 1.2 equiv) and K₂CO₃ (50.17 mg, 0.365 mmol, 5 equiv) in MeCN (1 mL) was stirred for 10 min at room temperature under air atmosphere. To the above mixture was added 159-3 (50 mg, 0.073 mmol, 1 equiv) and KI (12.05 mg, 0.438 mmol, 1 equiv). The resulting mixture was stirred for additional 1 h at 60° C. Desired product could be detected by LCMS. The resulting mixture was concentrated under reduced pressure to afford crude product which was further purified by reversed-phase flash chromatography with the following conditions: column, C18 silica gel; mobile phase, MeCN in Water (10 mmol/L NH₄HCO3), 30% to 70% gradient in 25 min to afford 159-4 (20 mg, 29.46%) as a white solid. LCMS (ESI, m/z): [M+H]⁺=935.

### Step E. 1-[6-(1-{[3-(4-{7′-[(1R,3R)-3-Hydroxycyclohexyl]-6′-oxospiro [cyclopropane-1,5′-pyrrolo[2,3-d]pyrimidin]-2′-ylamino}piperidin-1-ylsulfonyl)-2-methylphenyl]methyl}piperidin-4-yl)-1-methylindazol-3-yl]-1,3-diazinane-2,4-dione (159)

To a stirred solution of 159-4 (20 mg, 0.021 mmol, 1 equiv) in MeCN (2 mL) was added 4M HCl (gas) in 1,4-dioxane (2 mL) dropwise at 0° C. The resulting mixture was stirred for 1 h at 0° C. Desired product could be detected by LCMS. The resulting mixture was concentrated under reduced pressure to afford crude product which was further purified by reversed-phase flash chromatography with the following conditions: column, C18 silica gel; mobile phase, MeCN in Water (10 mmol/L NH₄HCO₃), 35% to 70% gradient in 30 min to afford compound 159 (8.1 mg, 43.88%) as a white solid. LCMS (ESI, m/z): [M+H]⁺=851.3.

### Example 160: 1-[6-(1-{[4-(4-{7′-[(1R,3R)-3-Hydroxycyclohexyl]-6′-oxospiro[cyclopropane-1,5′-pyrrolo[2,3-d]pyrimidin]-2′-ylamino}piperidin-1-ylsulfonyl)pyridin-2-yl]methyl}piperidin-4-yl)-1-methylindazol-3-yl]-1,3-diazinane-2,4-dione (160)

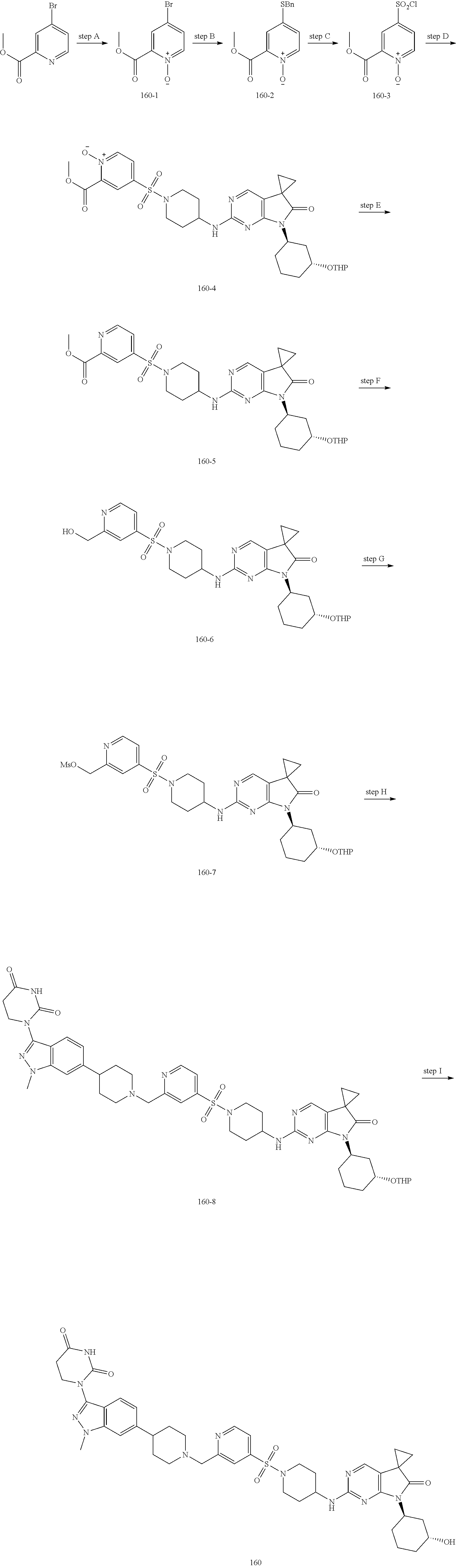

### Step A. 4-Bromo-2-(methoxycarbonyl) pyridin-1-ium-1-olate (160-1)

A solution of methyl 4-bromopyridine-2-carboxylate (3 g, 13.887 mmol, 1 equiv) and M-CPBA (7.19 g, 41.661 mmol, 3 equiv) in DCM (30 mL) was stirred for 1 h at 0° C. Desired product could be detected by LCMS. The reaction was quenched with Water at room temperature. The aqueous layer was extracted with CH₂Cl₂ (3×50 mL). The combined organic layers were washed with brine, dried over anhydrous Na₂SO₄. The resulting mixture was concentrated under reduced pressure. The residue was purified by silica gel column chromatography, eluted with PE/EA (1:1) to afford 160-1 (2.4 g, 74.48%) as a white solid. LCMS (ESI, m/z): [M+H]⁺=232.

### Step B. 4-(Benzylsulfanyl)-2-(methoxycarbonyl) pyridin-1-ium-1-olate (160-2)

A solution of 160-1 (1.5 g, 6.465 mmol, 1 equiv), benzyl mercaptan (963.48 mg, 7.758 mmol, 1.2 equiv), Pd₂(dba)₃ (591.98 mg, 0.647 mmol, 0.1 equiv), Xantphos (374.06 mg, 0.647 mmol, 0.1 equiv) and DIEA (1.67 g, 12.930 mmol, 2 equiv) in Toluene (15 mL) was stirred for 1 h at 80° C. under argon atmosphere. Desired product could be detected by LCMS. The reaction was quenched with water at room temperature. The aqueous layer was extracted with EtOAc (3×20 mL). The combined organic layers were washed with brine, dried over anhydrous Na₂SO₄. The resulting mixture was concentrated under reduced pressure. The residue was purified by silica gel column chromatography, eluted with PE/EA (5:1) to afford 160-2 (1.6 g, 89.90%) as a yellow solid. LCMS (ESI, m/z): [M+H]⁺=276.

### Step C. 4-(Chlorosulfonyl)-2-(methoxycarbonyl) pyridin-1-ium-1-olate (160-3)

To a stirred solution of 160-2 (500 mg, 1.816 mmol, 1 equiv) and AcOH (6 mL) in H₂O (2 mL) was added NCS (1.16 g, 7.264 mmol, 4 equiv) dropwise at 0° C. under nitrogen atmosphere. The resulting mixture was stirred for 1 h at 0° C. under nitrogen atmosphere. The reaction was monitored by TLC (PE:EA=1:1). The residue was dissolved in DCM (20 mL). The resulting mixture was washed with 3×5 mL of H₂O. The combined organic layers were washed with brine, dried over anhydrous Na₂SO₄. After filtration, the filtrate was concentrated under reduced pressure. This resulted in 160-3 (200 mg, 43.76%) as a yellow oil. LCMS (ESI, m/z): [M+H]⁺=252.

### Step D. 2-(Methoxycarbonyl)-4-(4-{7′-[(1R,3R)-3-(oxan-2-yloxy) cyclohexyl]-6′-oxospiro[cyclopropane-1,5′-pyrrolo[2,3-d]pyrimidin]-2′-ylamino}piperidin-1-ylsulfonyl) pyridin-1-ium-1-olate (160-4)

To a solution of DIEA (175.62 mg, 1.359 mmol, 3.00 equiv) and 7b (200 mg, 0.453 mmol, 1.00 equiv) in DCM (5 mL) was added 160-3 (113.97 mg, 0.453 mmol, 1.00 equiv) dropwise at 0° C. under nitrogen atmosphere. The resulting mixture was stirred for 1 h at 0° C. under nitrogen atmosphere. Desired product could be detected by LCMS. The residue was purified by reversed-phase flash chromatography with the following conditions: column, C18 silica gel; mobile phase, MeCN in Water, 10% to 60% gradient in 10 min; detector, UV 254 nm. This resulted in 160-4 (200 mg, 67.24%) as a yellow solid. LCMS (ESI, m/z): [M+H]⁺=657.

### Step E. Methyl 4-(4-{7′-[(1R,3R)-3-(oxan-2-yloxy)cyclohexyl]-6′-oxospiro[cyclopropane-1,5′-pyrrolo[2,3-d]pyrimidin]-2′-ylamino}piperidin-1-ylsulfonyl)pyridine-2-carboxylate (160-5)

To a solution of 160-4 (288 mg, 0.439 mmol, 1 equiv) in CF₃CH₂OH (10 mL) was added Pd/C (144.20 mg) under nitrogen atmosphere in a 50 mL round-bottom flask. The mixture was hydrogenated at room temperature for 1 h under hydrogen atmosphere using a hydrogen balloon, filtered through a Celite pad and concentrated under reduced pressure. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. This resulted in 160-5 (120 mg, 42.71%) as a white solid. LCMS (ESI, m/z): [M+H]⁺=641.

### Step F. 2′-({1-[2-(Hydroxymethyl)pyridin-4-ylsulfonyl]piperidin-4-yl}amino)-7′-[(1R,3R)-3-(oxan-2-yloxy)cyclohexyl]spiro[cyclopropane-1,5′-pyrrolo[2,3-d]pyrimidin]-6′-one (160-6)

To a stirred solution of 160-5 (220 mg, 0.343 mmol, 1.00 equiv) and NaOMe (18.55 mg, 0.343 mmol, 1.00 equiv) in MeOH (3 mL) was added NaBH₄ (25.98 mg, 0.686 mmol, 2 equiv) at room temperature under nitrogen atmosphere. The resulting mixture was stirred for 1 h at room temperature under nitrogen atmosphere. Desired product could be detected by LCMS. The residue was purified by reversed-phase flash chromatography with the following conditions: column, C18 silica gel; mobile phase, MeCN in Water (10 mmol/L NH₄HCO₃), 10% to 60% gradient in 10 min; detector, UV 254 nm. This resulted in 160-6 (120 mg, 57.04%) as a white solid. LCMS (ESI, m/z): [M+H]⁺=613.

### Step G-I. 1-[6-(1-{1-[4-(4-{7′-[(1R,3R)-3-Hydroxycyclohexyl]-6′-oxospiro[cyclopropane-1,5′-pyrrolo[2,3-d]pyrimidin]-2′-ylamino}piperidin-1-ylsulfonyl)pyridin-2-yl]methyl}piperidin-4-yl)-1-methylindazol-3-yl]-1,3-diazinane-2,4-dione (160)

Compound 160 was prepared from 160-6 and Int-24 following the procedure for the synthesis of compound 151-6 in example 151. LCMS (ESI, m/z): [M+H]⁺=838.3.

### Example 161: 1-[6-(1-{[5-(4-{7′-[(1S,3S)-3-Hydroxycyclohexyl]-6′-oxospiro[cyclopropane-1,5′-pyrrolo[2,3-d]pyrimidin]-2′-ylamino}piperidin-1-ylsulfonyl)pyridin-3-yl]methyl}piperidin-4-yl)-1-methylindazol-3-yl]-1,3-diazinane-2,4-dione (161)

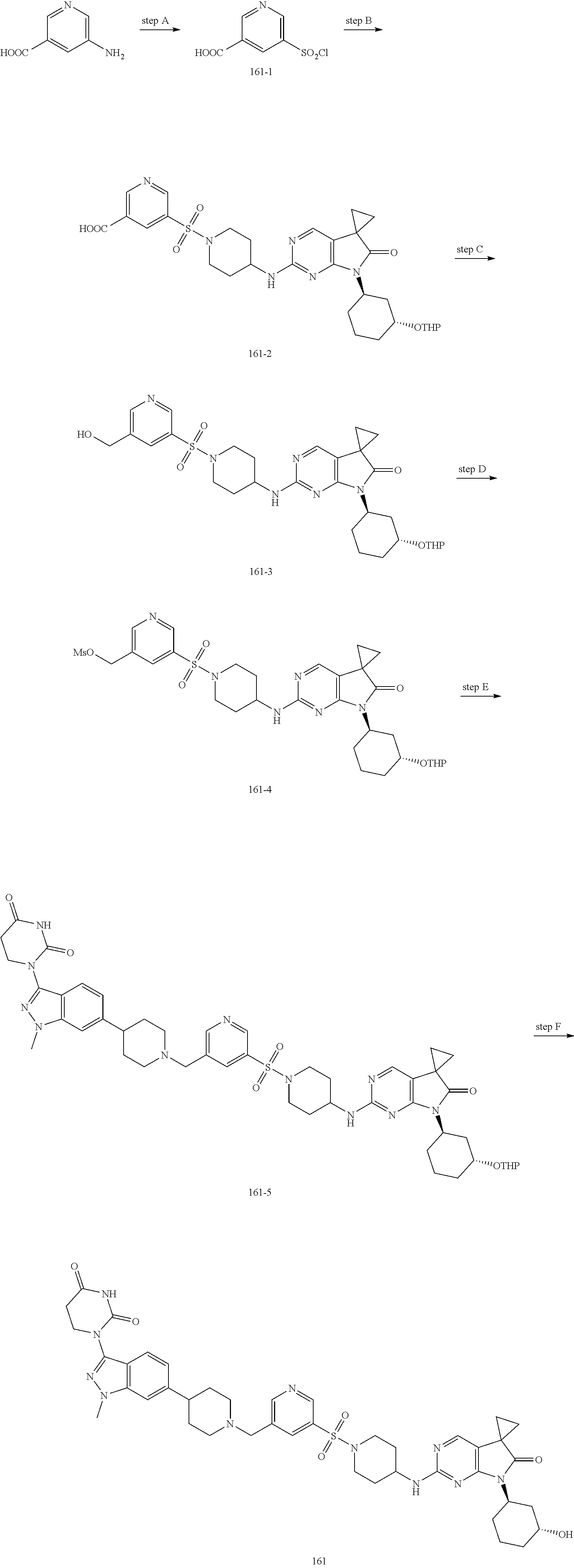

### Step A. 5-(Chlorosulfonyl)pyridine-3-carboxylic acid (161-1)

To a mixture of 5-aminopyridine-3-carboxylic acid (500 mg, 3.620 mmol, 1 equiv) and conc.HCl (3 mL) in H₂O (10 mL) were added NaNO₂ (299.70 mg, 4.344 mmol, 1.2 equiv) at 0 degrees C. under N₂. The resulting mixture was stirred for 1 h at 0 degrees C. To the above mixture was added SOCl₂ (3444.97 mg, 28.960 mmol, 8 equiv) and CuCl (35.84 mg, 0.362 mmol, 0.1 equiv) in H₂O (10 mL) dropwise over 10 min at 0 degrees C. The resulting mixture was stirred for additional 1 h at 0 degrees C. Desired product could be detected by LCMS. The resulting mixture was diluted with water (50 mL) and extracted with EtOAc (3×100 mL). The combined organic layers were dried over anhydrous Na₂SO₄ and concentrated under reduced pressure. The residue was purified by reversed-phase Combi-Flash, eluted with CH₃CN:H₂O (0.5% NH₄HCO₃)=5%-80%. This resulted in 161-1 (265 mg, 29.73%) as a yellow solid. LCMS (ESI, m/z): [M+H]⁺=222.

### Step B. 5-(4-{7′-[(1S,3S)-3-(Oxan-2-yloxy)cyclohexyl]-6′-oxospiro[cyclopropane-1,5′-pyrrolo[2,3-d]pyrimidin]-2′-ylamino}piperidin-1-ylsulfonyl)pyridine-3-carboxylic acid (161-2)

To a mixture of 161-1 (200 mg, 0.902 mmol, 1 equiv) and 7b (398.52 mg, 0.902 mmol, 1 equiv) in DCM (10 mL) were added TEA (273.98 mg, 2.706 mmol, 3 equiv) at 0 degrees C. The resulting mixture was stirred for 1 h at 25 degrees C. Desired product could be detected by LCMS. The resulting mixture was diluted with water (5 mL) and extracted with EtOAc (3×30 mL). The combined organic layers were dried over anhydrous Na₂SO₄ and concentrated under reduced pressure. The residue was purified by reversed-phase Combi-Flash, eluted with CH₃CN:H₂O (0.5% NH₄HCO₃)=5%-65%. This resulted in 161-2 (232 mg, 35.27%) as a yellow solid. LCMS (ESI, m/z): [M+H]⁺=627.

### Step C. 2′-({1-[5-(Hydroxymethyl)pyridin-3-ylsulfonyl]piperidin-4-yl}amino)-7′-[(1S,3S)-3-(oxan-2-yloxy)cyclohexyl]spiro[cyclopropane-1,5′-pyrrolo[2,3-d]pyrimidin]-6′-one (161-3)

To a stirred solution of 161-2 (150 mg, 0.239 mmol, 1 equiv) in THF (8 mL) were added CDI (155.24 mg, 0.956 mmol, 4 equiv) at 25 degrees C. under N₂. The resulting mixture was stirred for 1 h at 25 degrees C. To the above mixture was added NaBH₄ (36.22 mg, 0.956 mmol, 4 equiv) in H₂O (10 mL) and THF (16 mL) at 25 degrees C. The resulting mixture was stirred for additional 1 h at 25 degrees C. Desired product could be detected by LCMS. The resulting mixture was diluted with water (50 mL) and extracted with EtOAc (3×100 mL). The combined organic layers were dried over anhydrous Na₂SO₄ and concentrated under reduced pressure. The residue was purified by reversed-phase Combi-Flash, eluted with CH₃CN:H₂O (0.5% NH₄HCO₃)=5%-65%. This resulted in 161-3 (45 mg, 29.15%) as a yellow solid. LCMS (ESI, m/z): [M+H]⁺=613.

### Step D-F. 1-[6-(1-{1-[5-(4-{7′-[(1S,3S)-3-Hydroxycyclohexyl]-6′-oxospiro[cyclopropane-1,5′-pyrrolo[2,3-d]pyrimidin]-2′-ylamino}piperidin-1-ylsulfonyl)pyridin-3-yl]methyl}piperidin-4-yl)-1-methylindazol-3-yl]-1,3-diazinane-2,4-dione (161)

Compound 161 was prepared from 161-3 and Int-24 following the procedure for the synthesis of compound 151-6 in example 151. LCMS (ESI, m/z): [M+H]⁺=838.4.

### Example 162: 1-(6-{1-[(3-{4-[7′-(2-Chloro-5-fluorophenyl)-6′-oxospiro[cyclopropane-1,5′-pyrrolo[2,3-d]pyrimidin]-2′-ylamino}piperidin-1-ylsulfonyl}phenyl)methyl]piperidin-4-yl]-1-methylindazol-3-yl)-1,3-diazinane-2,4-dione (162)

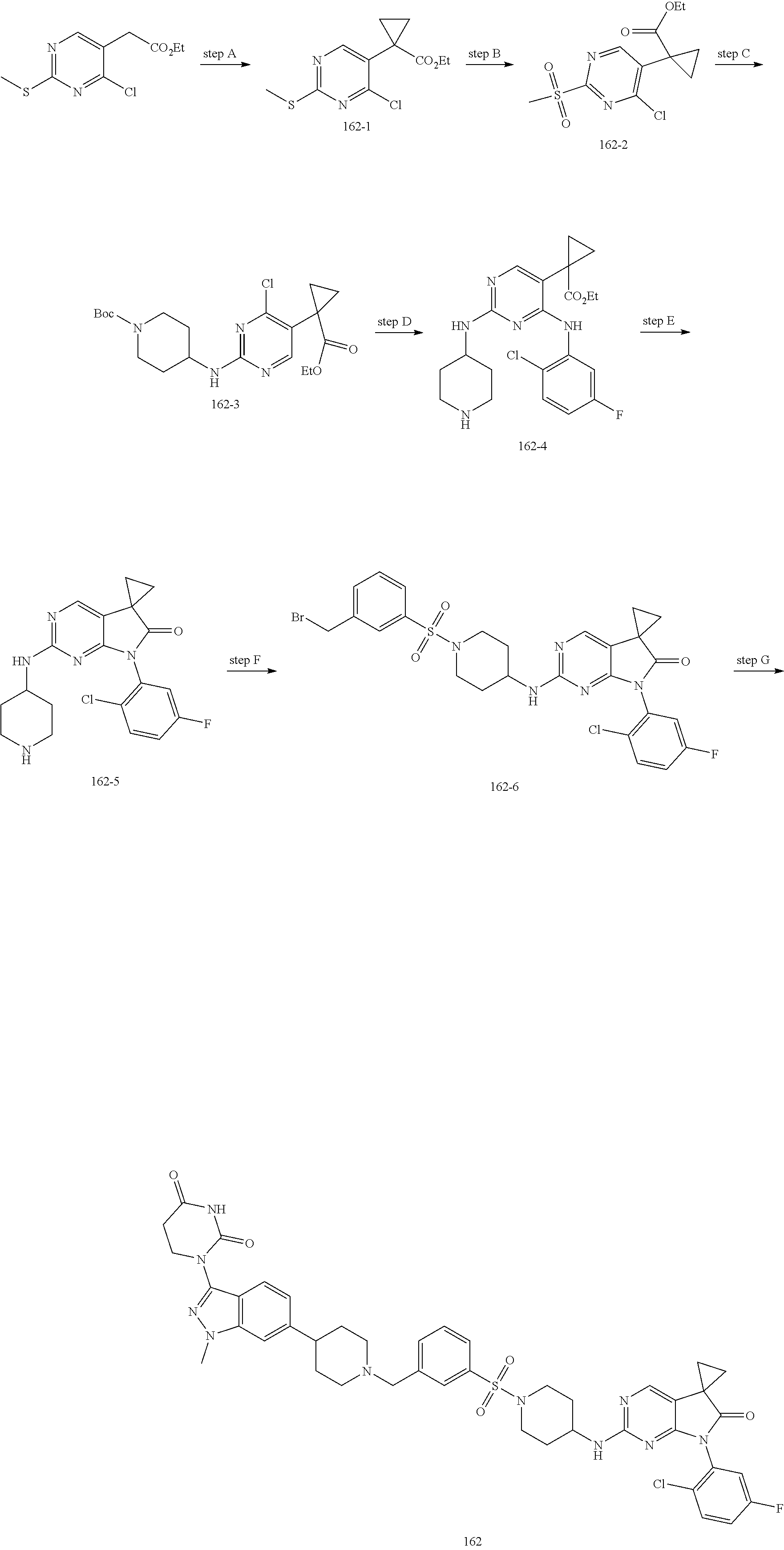

### Step A. Ethyl 1-[4-chloro-2-(methylsulfanyl)pyrimidin-5-yl]cyclopropane-1-carboxylate (162-1)

A mixture of ethyl 2-[4-chloro-2-(methylsulfanyl)pyrimidin-5-yl]acetate (10 g, 40.533 mmol, 1 equiv) and dibromoethane (9137.63 mg, 48.640 mmol, 1.2 equiv) and NaH (4053.32 mg, 101.333 mmol, 2.5 equiv, 60%) in DMF (100 mL) was stirred for 1 h at 0° C. under argon atmosphere. The reaction was quenched with sat. NH₄C1 (aq.) at 0° C. The aqueous layer was extracted with EtOEt (3×100 mL). The combined organic layers were dried over anhydrous Na₂SO₄ and concentrated under reduced pressure. The residue was purified by silica gel column chromatography, eluted with PE/EA (5:1) to afford 162-1 (5.5 g, 49.75%) as a yellow solid. LCMS (ESI, m/z): [M+H]⁺=273.

### Step B. Ethyl 1-(4-chloro-2-methanesulfonylpyrimidin-5-yl)cyclopropane-1-carboxylate (162-2)

A solution of 162-1 (15.5 g, 56.829 mmol, 1 equiv), Oxone (28.67 g, 170.487 mmol, 3 equiv) and H₂O (30 mL) in THF (150 mL) was stirred for 3 h at room temperature. The aqueous layer was extracted with EtOEt (3×100 mL). The resulting mixture was concentrated under reduced pressure. This resulted in 162-2 (15.5 g, 89.50%) as a white solid. LCMS (ESI, m/z): [M+H]⁺=305.

### Step C. Tert-butyl 4-({4-chloro-5-[1-(ethoxycarbonyl)cyclopropyl]pyrimidin-2-yl}amino)piperidine-1-carboxylate (162-3)

A solution of 162-2 (4 g, 13.126 mmol, 1 equiv), tert-butyl 4-aminopiperidine-1-carboxylate (2.63 g, 13.126 mmol, 1 equiv) and NaH (577.56 mg, 14.439 mmol, 1.1 equiv) in THF (50 mL) was stirred for 1 h at 80° C. under air atmosphere. Desired product could be detected by LCMS. The reaction was quenched with water at room temperature. The aqueous layer was extracted with EtOAc (3×50 mL). The combined organic layers were dried over anhydrous Na₂SO₄ and concentrated under reduced pressure. The residue was purified by silica gel column chromatography, eluted with PE/EA (3:1) to afford 162-3 (1.2 g, 21.52%) as a white solid. LCMS (ESI, m/z): [M+H]⁺=425.

### Step D. Ethyl 1-{4-[(2-chloro-5-fluorophenyl)amino]-2-(piperidin-4-ylamino)pyrimidin-5-yl}cyclopropane-1-carboxylate (162-4)

To a solution of 162-3 (200 mg, 0.471 mmol, 1 equiv) in CF₃CH₂OH (2 mL) was added 2-chloro-5-fluoroaniline (342.55 mg, 2.355 mmol, 5 equiv), TFA (536.67 mg, 4.710 mmol, 10 equiv) at room temperature. The mixture was stirred for overnight at 120° C. Desired product could be detected by LCMS. The mixture was neutralized to pH 8 with sat. Na₂CO₃ (aq.). The aqueous layer was extracted with EtOAc (3×50 mL). The combined organic layers were dried over anhydrous Na₂SO₄ and concentrated under reduced pressure. The residue was purified by reversed-phase flash chromatography with the following conditions: column, C18 silica gel; mobile phase, water in ACN, 10% to 50% gradient in 25 min; detector, UV 254 nm. This resulted in 162-4 (120 mg, 58.76%) as white solid. LCMS (ESI, m/z): [M+H]⁺=434.

### Step E. 7′-(2-Chloro-5-fluorophenyl)-2′-(piperidin-4-ylamino)spiro[cyclopropane-1,5′-pyrrolo[2,3-d]pyrimidin]-6′-one (162-5)

To a solution of 162-4 (100 mg, 0.230 mmol, 1 equiv) in THF (2 mL) was added NaH (18.4 mg, 0.460 mmol, 2 equiv) at 0° C. The mixture was stirred for 1 hour. Desired product could be detected by LCMS. The resulting mixture was concentrated under reduced pressure. The residue was purified by silica gel column chromatography, eluted with DCM/MeOH (30/1) to afford 162-5 (60 mg, 67.13%) as a white solid. LCMS (ESI, m/z): [M+H]⁺=388.

### Step F-G. 1-(6-{1-[(3-{4-[7′-(2-Chloro-5-fluorophenyl)-6′-oxospiro[cyclopropane-1,5′-pyrrolo[2,3-d]pyrimidin]-2′-ylamino]piperidin-1-ylsulfonyl}phenyl)methyl]piperidin-4-yl}-1-methylindazol-3-yl)-1,3-diazinane-2,4-dione (162)

Compound 162 was prepared from 162-5 and Int-24 following the procedure for the synthesis of compound 118 in example 118. LCMS (ESI, m/z): [M+H]⁺=867.3.

### Example 163: 1-[6-(1-{[3-(4-{7′-[(1R,3R)-3-Hydroxycyclohexyl]-6′-oxospiro [cyclopropane-1,5′-pyrrolo[2,3-d] pyrimidin]-2′-ylamino} piperidin-1-ylsulfonyl)-4-methylphenyl] methyl} piperidin-4-yl)-1-methylindazol-3-yl]-1,3-diazinane-2,4-dione (163)

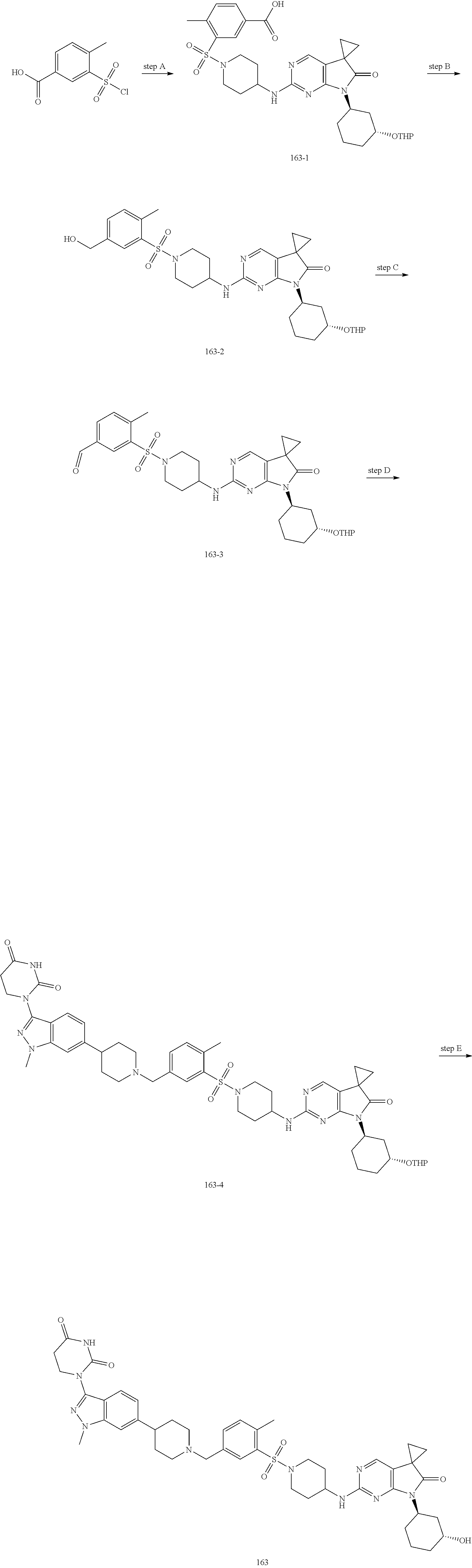

### Step A-B. 2′-{[1-(5-Hydroxy-2-methylbenzenesulfonyl) piperidin-4-yl]amino}-7′-[(1R,3R)-3-(oxan-2-yloxy) cyclohexyl] spiro[cyclopropane-1,5′-pyrrolo[2,3-d]pyrimidin]-6′-one (163-2)

Compound 163-2 was prepared from 7b following the procedure for the synthesis of compound 161-3 in example 161. LCMS (ESI, m/z): [M+H]*=626.

### Step C. Tert-butyl 4-({4-chloro-5-[1-(ethoxycarbonyl)cyclopropyl]pyrimidin-2-yl}amino)piperidine-1-carboxylate (163-3)

A solution of 163-2 (90 mg, 0.147 mmol, 1 equiv) and manganese dioxide (255.79 mg, 2.940 mmol, 20 equiv) in DCM (2 mL) was stirred for 12 h at 50° C. under nitrogen atmosphere. Desired product could be detected by LCMS. The resulting mixture was diluted with DCM (30 mL). After filtration, the filtrate was concentrated under reduced pressure to afford 163-3 (50 mg, 54.49%) as a light yellow crude oil. The crude product was used directly for next step. L CMS (ESI, m/z): [M+H]⁺=624.

### Step D. 1-[1-Methyl-6-(1-{[4-methyl-3-(4-{7′-[(1R,3R)-3-(oxan-2-yloxy)-cyclohexyl]-6′-oxospiro [cyclopropane-1,5′-pyrrolo[2,3-d]pyrimidin]-2′-ylamino}piperidin-1-ylsulfonyl) phenyl]methyl}piperidin-4-yl) indazol-3-yl]-1,3-diazinane-2,4-dione (163-4)

To a stirred solution of Int-24 (31.49 mg, 0.096 mmol, 1.2 equiv) and NaOAc (13.15 mg, 0.160 mmol, 2 equiv) in CH₃OH (2 mL) was added CH₃COOH (14.44 mg, 0.240 mmol, 3 equiv) and 163-3 (50 mg, 0.080 mmol, 1 equiv) at room temperature. The resulting mixture was stirred for 10 min at room temperature. To the above mixture was added NaBH₃CN (10.07 mg, 0.160 mmol, 2 equiv). The resulting mixture was stirred for additional 1 h at room temperature. Desired product could be detected by LCMS. The resulting mixture was concentrated under reduced pressure to afford crude product which was further purified by reversed-phase flash chromatography with the following conditions: column, C18 silica gel; mobile phase, MeCN in Water (10 mmol/L NH₄HCO₃), 30% to 70% gradient in 25 min to afford 163-4 (30 mg, 26.68%) as a white solid. LCMS (ESI, m/z): [M+H]⁺=935.

### Step E. 1-[6-(1-{[3-(4-{7′-[(1R,3R)-3-Hydroxycyclohexyl]-6′-oxospiro [cyclopropane-1,5′-pyrrolo[2,3-d]pyrimidin]-2′-ylamino}piperidin-1-ylsulfonyl)-4-methylphenyl]methyl}piperidin-4-yl)-1-methylindazol-3-yl]-1,3-diazinane-2,4-dione (163)

To a stirred solution of 163-4 (30 mg, 0.032 mmol, 1 equiv) in MeCN (1 mL) was added 4M HCl (gas) in 1,4-dioxane (1 mL) dropwise at 0° C. under air atmosphere. The resulting mixture was stirred for 30 min at 0° C. under air atmosphere. Desired product could be detected by LCMS. The resulting mixture was concentrated under reduced pressure to afford crude product which was further residue was purified by reversed-phase flash chromatography with the following conditions: column, C18 silica gel; mobile phase, MeCN in Water (10 mmol/L NH₄HCO₃), 35% to 70% gradient in 35 min to afford compound 163 (5.6 mg, 20.51%) as a white solid. LCMS (ESI, m/z): [M+H]⁺=851.3

### Example 164: 1-[6-(1-{[3-(4-{7′-[(1S)-1-Cyclopropylethyl]-6′-oxospiro[cyclopropane-1,5′-pyrrolo[2,3-d]pyrimidin]-2′-ylamino}piperidin-1-ylsulfonyl)phenyl]methyl}piperidin-4-yl)-1-methylindazol-3-yl]-1,3-diazinane-2,4-dione (164)

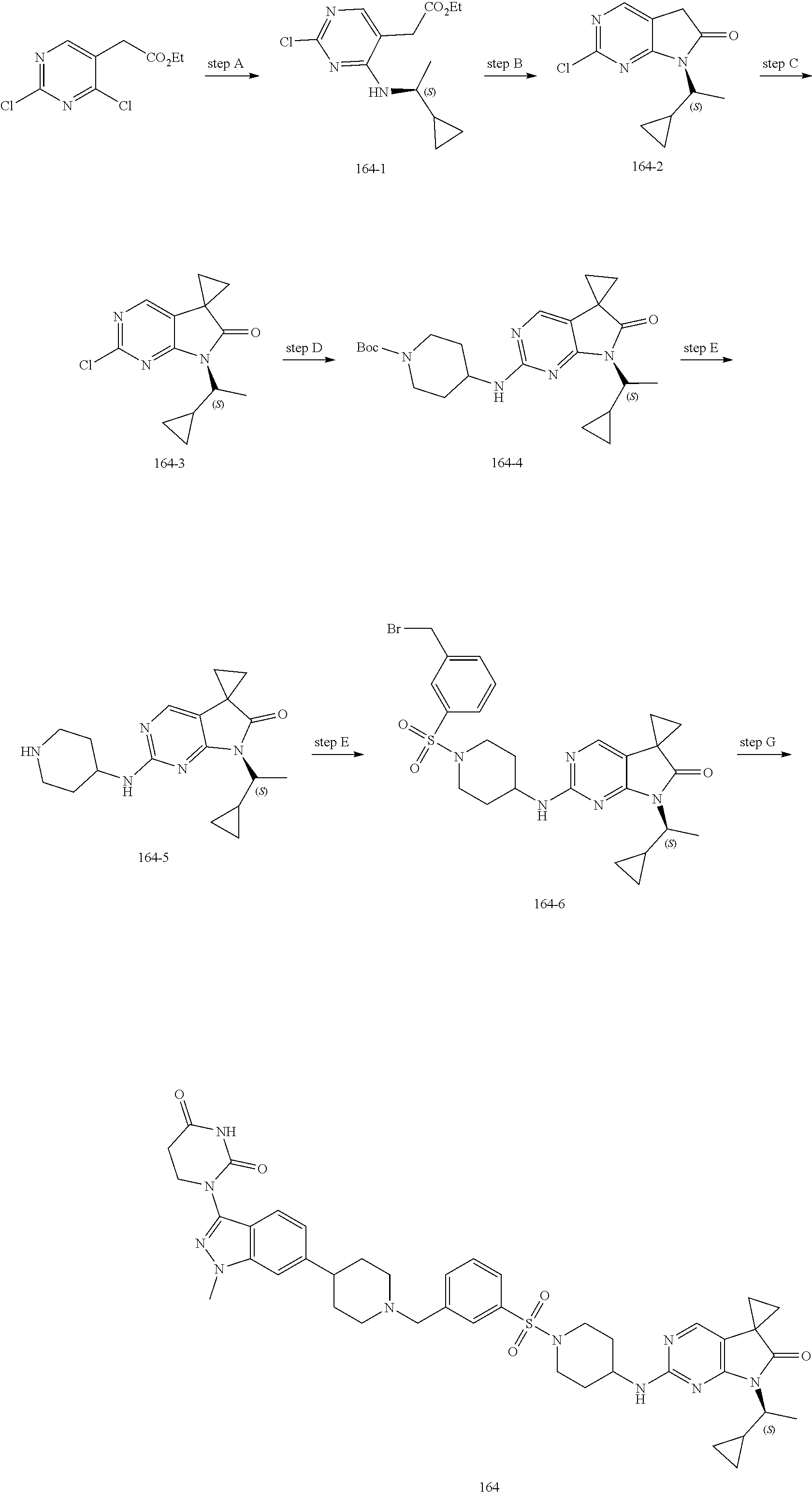

### Step A. Ethyl 2-(2-chloro-4-{[(1S)-1-cyclopropylethyl]amino}pyrimidin-5-yl)acetate (164-1)

To a stirred mixture of ethyl 2-(2,4-dichloropyrimidin-5-yl)acetate (2 g, 8.508 mmol, 1 equiv) and (1S)-1-cyclopropylethanamine hydrochloride (1.24 g, 10.210 mmol, 1.2 equiv) in ACN (20 mL) was added DIPEA (3.30 g, 25.524 mmol, 3 equiv). The resulting mixture was stirred for 1 h at 80° C. The reaction was quenched by the addition of water (10 mL). The resulting mixture was extracted with EtOAc. The combined organic layers were dried over anhydrous Na₂SO₄. After filtration, the filtrate was concentrated under reduced pressure. The residue was purified by silica gel column chromatography, eluted with PE/EA (3:1) to afford 164-1 (1.2 g, 46.67%) as a colorless oil. LCMS (ESI, m/z): [M+H]⁺=284.

### Step B-G. 1-[6-(1-{[3-(4-{7′-[(1S)-1-Cyclopropylethyl]-6′-oxospiro[cyclopropane-1,5′-pyrrolo[2,3-d]pyrimidin]-2′-ylamino}piperidin-1-ylsulfonyl)phenyl]methyl}piperidin-4-yl)-1-methylindazol-3-yl]-1,3-diazinane-2,4-dione (164)

Compound 164 was prepared from 164-1 and Int-24 following the procedure for the synthesis of Int 6 and compound 118 in example 118. LCMS (ESI, m/z): [M+H]⁺=807.4.

### Example 165: 1-[6-(1-{[2-(4-{7′-[(1R,3R)-3-Hydroxycyclohexyl]-6′-oxospiro[cyclopropane-1,5′-pyrrolo[2,3-d]pyrimidin]-2′-ylamino}piperidin-1-ylsulfonyl)pyridin-4-yl]methyl}piperidin-4-yl)-1-methylindazol-3-yl]-1,3-diazinane-2,4-dione (165)

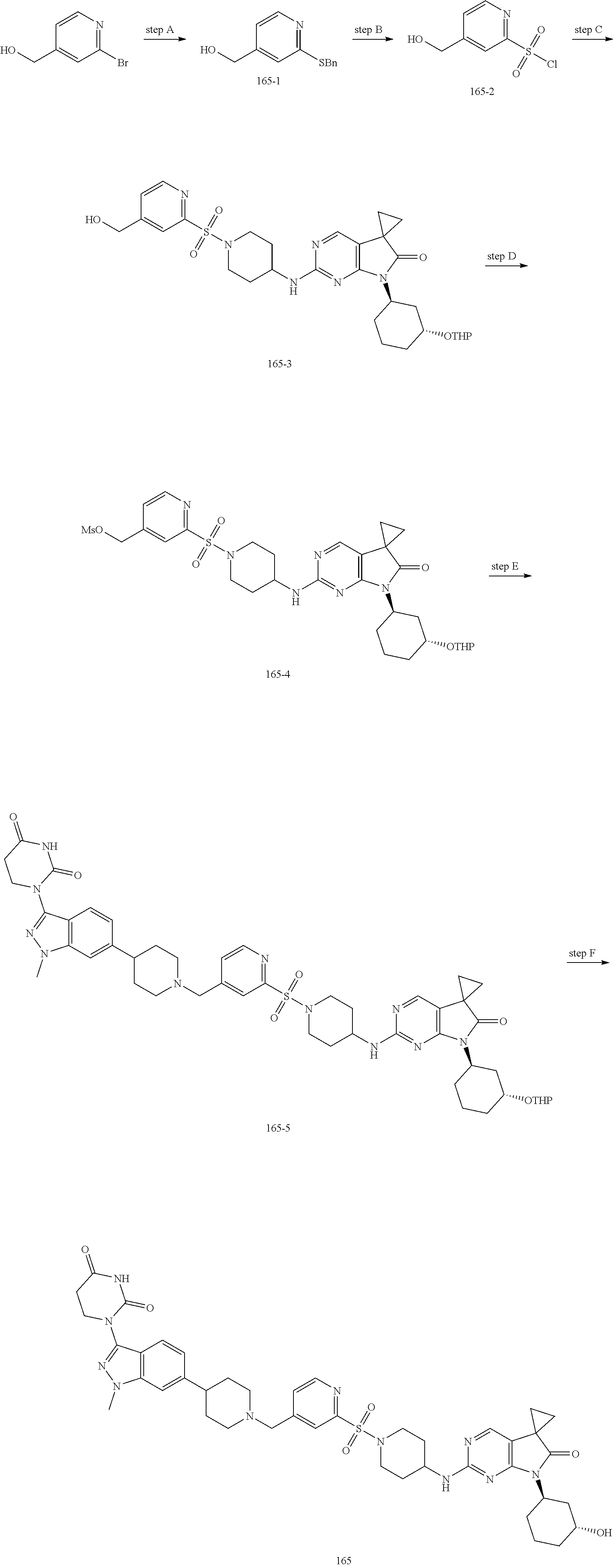

### Step A. [2-(Benzylsulfanyl)pyridin-4-yl]methanol (165-1)

To a stirred mixture of (2-bromopyridin-4-yl)methanol (3 g, 15.955 mmol, 1 equiv) and benzyl mercaptan (2.38 g, 19.146 mmol, 1.2 equiv) in toluene (15 mL) were added Pd₂(dba)₃ (0.58 g, 0.638 mmol, 0.04 equiv), Xantphos (0.74 g, 1.276 mmol, 0.08 equiv) and DIEA (4.54 g, 35.101 mmol, 2.2 equiv) at room temperature under argon atmosphere. The resulting mixture was stirred for 1 h at 80° C. under argon atmosphere. The resulting mixture was concentrated under reduced pressure. The residue was purified by silica gel column chromatography, eluted with PE/EA (3:1) to afford 165-1 (3 g, 81.29%) as a white solid. LCMS (ESI, m/z): [M+H]⁺=232.

### Step B. 4-(Hydroxymethyl)pyridine-2-sulfonyl chloride (165-2)

To a stirred mixture of 165-1 (500 mg, 2.162 mmol, 1 equiv) in AcOH (1.5 mL) and H₂O (0.5 mL) were added NCS (1376.58 mg, 8.648 mmol, 4 equiv) at room temperature. The resulting mixture was stirred for 1 h at room temperature. The reaction was monitored by LCMS. The resulting mixture was concentrated under reduced pressure. The crude product was used in the next step directly without further purification. LCMS (ESI, m/z): [M+H]⁺=208.

### Step C. 2′-({1-[4-(Hydroxymethyl)pyridin-2-ylsulfonyl]piperidin-4-yl}amino)-7′-[(1R,3R)-3-(oxan-2-yloxy)cyclohexyl]spiro[cyclopropane-1,5′-pyrrolo[2,3-d]pyrimidin]-6′-one (165-3)

To a mixture of 165-2 (100 mg, 0.482 mmol, 1 equiv) and DIEA (311.24 mg, 2.410 mmol, 5 equiv) in DCM (2 mL) was added 7b (191.41 mg, 0.434 mmol, 0.9 equiv) at 0° C. The resulting mixture was stirred for 1 h at room temperature. The reaction was monitored by LCMS. The resulting mixture was concentrated under vacuum. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. This resulted in 165-3 (40 mg, 13.55%) as a white solid. LCMS (ESI, m/z): [M+H]⁺=613.

### Step D-F. 1-[6-(1-{[2-(4-{7′-[(1R,3R)-3-Hydroxycyclohexyl]-6′-oxospiro[cyclopropane-1,5′-pyrrolo[2,3-d]pyrimidin]-2′-ylamino}piperidin-1-ylsulfonyl)pyridin-4-yl]methyl}piperidin-4-yl)-1-methylindazol-3-yl]-1,3-diazinane-2,4-dione (165)

Compound 165 was prepared from 165-3 and Int-24 following the procedure for the synthesis of compound 160 in example 160. LCMS (ESI, m/z): [M+H]⁺=838.3.

### Example 166: 1-[6-(1-{[6-(4-{7′-[(1R,3R)-3-Hydroxycyclohexyl]-6′-oxospiro[cyclopropane-1,5′-pyrrolo[2,3-d]pyrimidin]-2′-ylamino}piperidin-1-ylsulfonyl)pyridin-2-yl]methyl}piperidin-4-yl)-1-methylindazol-3-yl]-1,3-diazinane-2,4-dione (166)

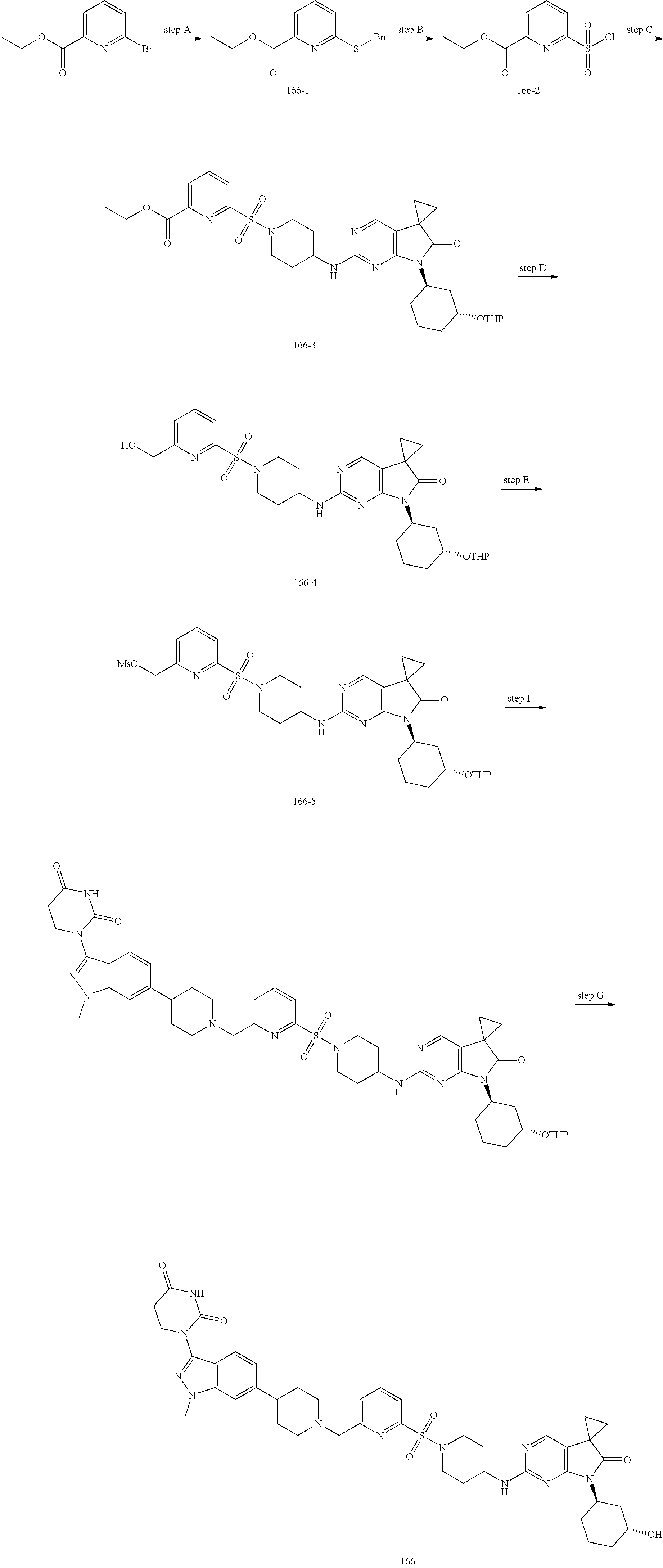

### Step A-C. Ethyl 6-(4-{7′-[(1R,3R)-3-(oxan-2-yloxy)cyclohexyl]-6′-oxospiro[cyclopropane-1,5′-pyrrolo[2,3-d]pyrimidin]-2′-ylamino}piperidin-1-ylsulfonyl)pyridine-2-carboxylate (166-3)

Compound 166-3 was prepared from 7b following the procedure for the synthesis of compound 165-3 in example 165. LCMS (ESI, m/z): [M+H]*=655.

### Step D-G. 1-[6-(1-{[6-(4-{7′-[(1R,3R)-3-Hydroxycyclohexyl]-6′-oxospiro[cyclopropane-1,5′-pyrrolo[2,3-d]pyrimidin]-2′-ylamino}piperidin-1-ylsulfonyl)pyridin-2-yl]meth yl}piperidin-4-yl)-1-methylindazol-3-yl]-1,3-diazinane-2,4-dione (166)

Compound 166 was prepared from 166-3 and Int 24 following the procedure for the synthesis of compound 160 in example 160. LCMS (ESI, m/z): [M+H]⁺=838.3.

### Example 167: 1-[6-(1-{[3-(4-{7′-[(1R,3R)-3-Hydroxycyclohexyl]-6′-oxospiro[cyclopropane-1,5′-pyrrolo[2,3-d]pyrimidin]-2′-ylamino}piperidin-1-ylsulfonyl)-5-methylphenyl]methyl}piperidin-4-yl)-1-methylindazol-3-yl]-1,3-diazinane-2,4-dione (167)

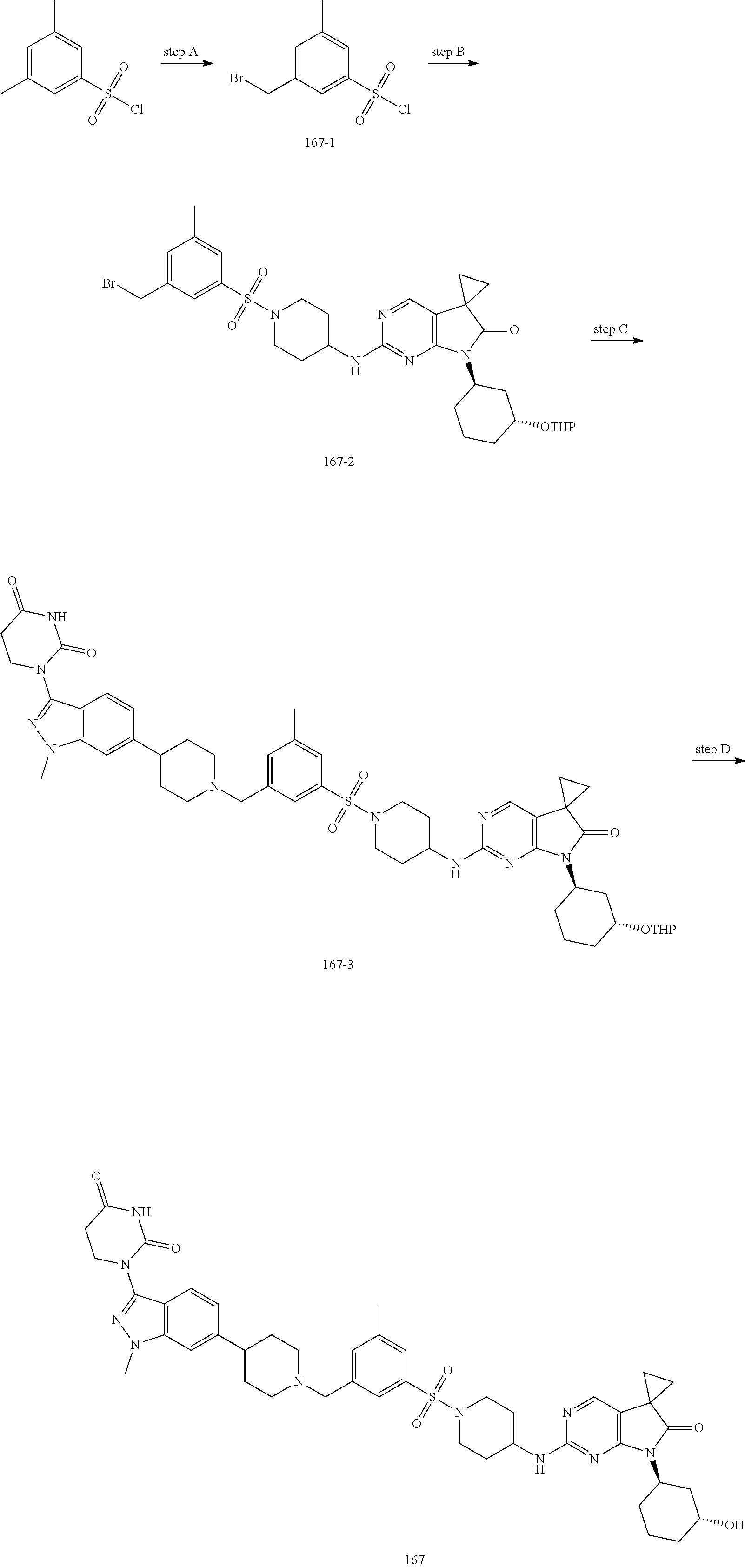

### Step A. 3-(Bromomethyl)-5-methylbenzenesulfonyl chloride (167-1)

To a stirred solution of 3,5-dimethylbenzenesulfonyl chloride (500 mg, 2.443 mmol, 1 equiv) in CCl₄ (5 mL) were added NBS (434.81 mg, 2.443 mmol, 1.0 equiv) and benzoyl peroxide (313.01 mg, 1.222 mmol, 0.5 equiv) at room temperature under nitrogen atmosphere. The resulting mixture was stirred for overnight at 80° C. under nitrogen atmosphere. Desired product could be detected by LCMS. The resulting mixture was concentrated under reduced pressure. The residue was purified by silica gel column chromatography, eluted with PE/EA (1:1) to afford 167-1 (205 mg, 23.67%) as a light yellow semi-solid.

### Step B-D. 3-(Bromomethyl)-5-methylbenzenesulfonyl chloride (167)

Compound 167 was prepared from 167-1, 7b and Int 24 following the procedure for the synthesis of compound 159 in example 159. LCMS (ESI, m/z): [M+H]⁺=851.4.

### Example 169: 1-[6-(1-{[2-Fluoro-5-(4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl)phenyl]methyl}piperidin-4-yl)-1-methylindazol-3-yl]-1,3-diazinane-2,4-dione (169)

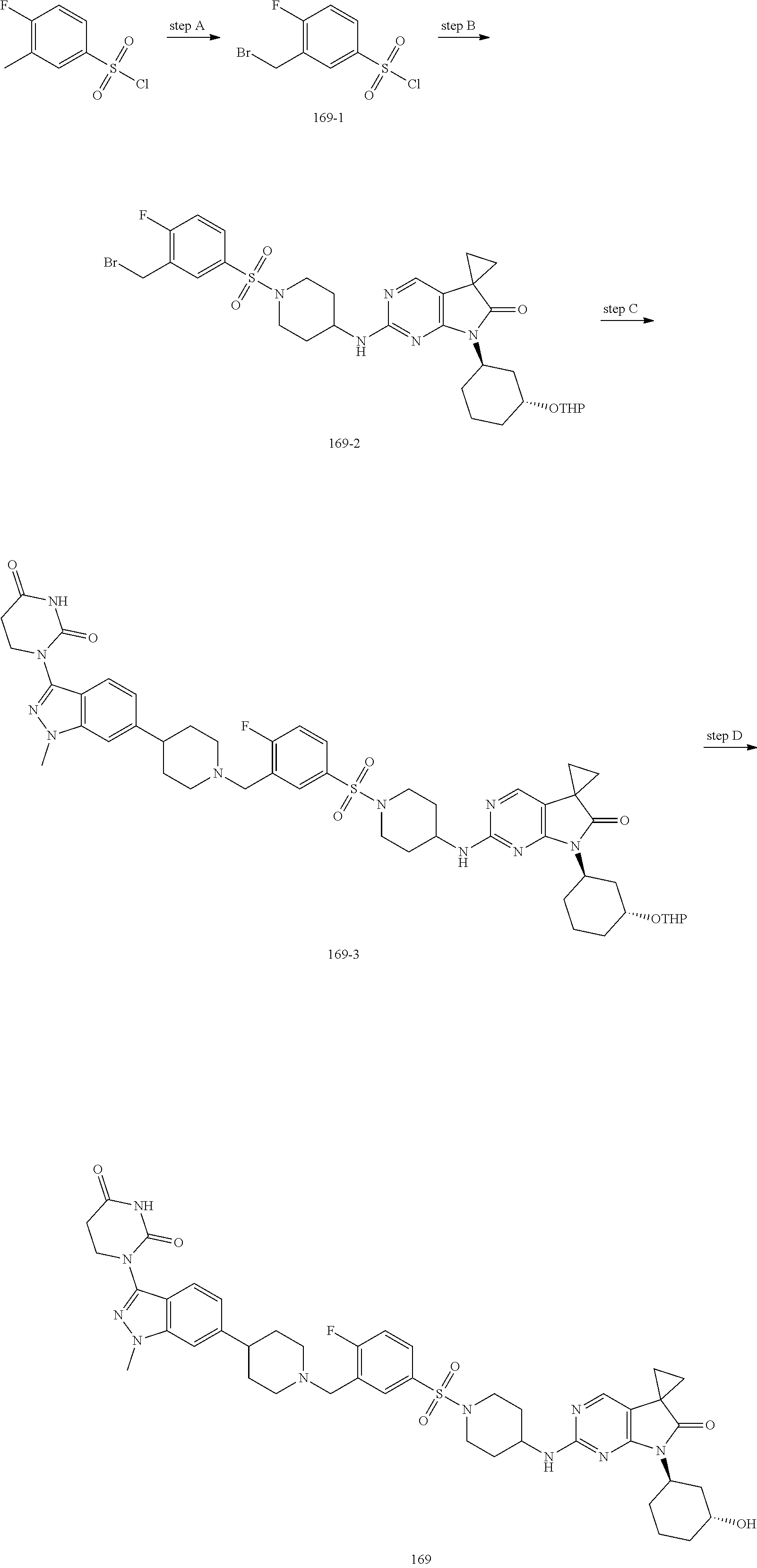

### Step A. 3-(Bromomethyl)-4-fluorobenzenesulfonyl chloride (170-1)

To a stirred mixture of 4-fluoro-3-methylbenzenesulfonyl chloride (400 mg, 1.917 mmol, 1 equiv) and NBS (307.12 mg, 1.725 mmol, 0.9 equiv) in ACN (5 mL) were added AIBN (31.48 mg, 0.192 mmol, 0.1 equiv) at room temperature under nitrogen atmosphere. The resulting mixture was stirred for 1 h at 80° C. under nitrogen atmosphere. Desired product could be detected by LCMS. The resulting mixture was diluted with water (20 mL). The resulting mixture was extracted with EtOAc (3×20 mL). The combined organic layers were washed with brine, dried over anhydrous Na₂SO₄. After filtration, the filtrate was concentrated under reduced pressure. The residue was purified by silica gel column chromatography, eluted with PE/EA (5:1) to afford 169-1 (230 mg, 41.72%) as a brown solid.

### Step B-D. 1-[6-(1-{[2-Fluoro-5-(4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl)phenyl]methyl}piperidin-4-yl)-1-methylindazol-3-yl]-1,3-diazinane-2,4-dione (169)

Compound 169 was prepared from 169-1, 7b and Int 24 following the procedure for the synthesis of compound 159 in example 159. LCMS (ESI, m/z): [M+H]⁺=855.5.

### Example 170: 1-[6-(1-{[3-Fluoro-5-(4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl)phenyl]methyl}piperidin-4-yl)-1-methylindazol-3-yl]-1,3-diazinane-2,4-dione (170)

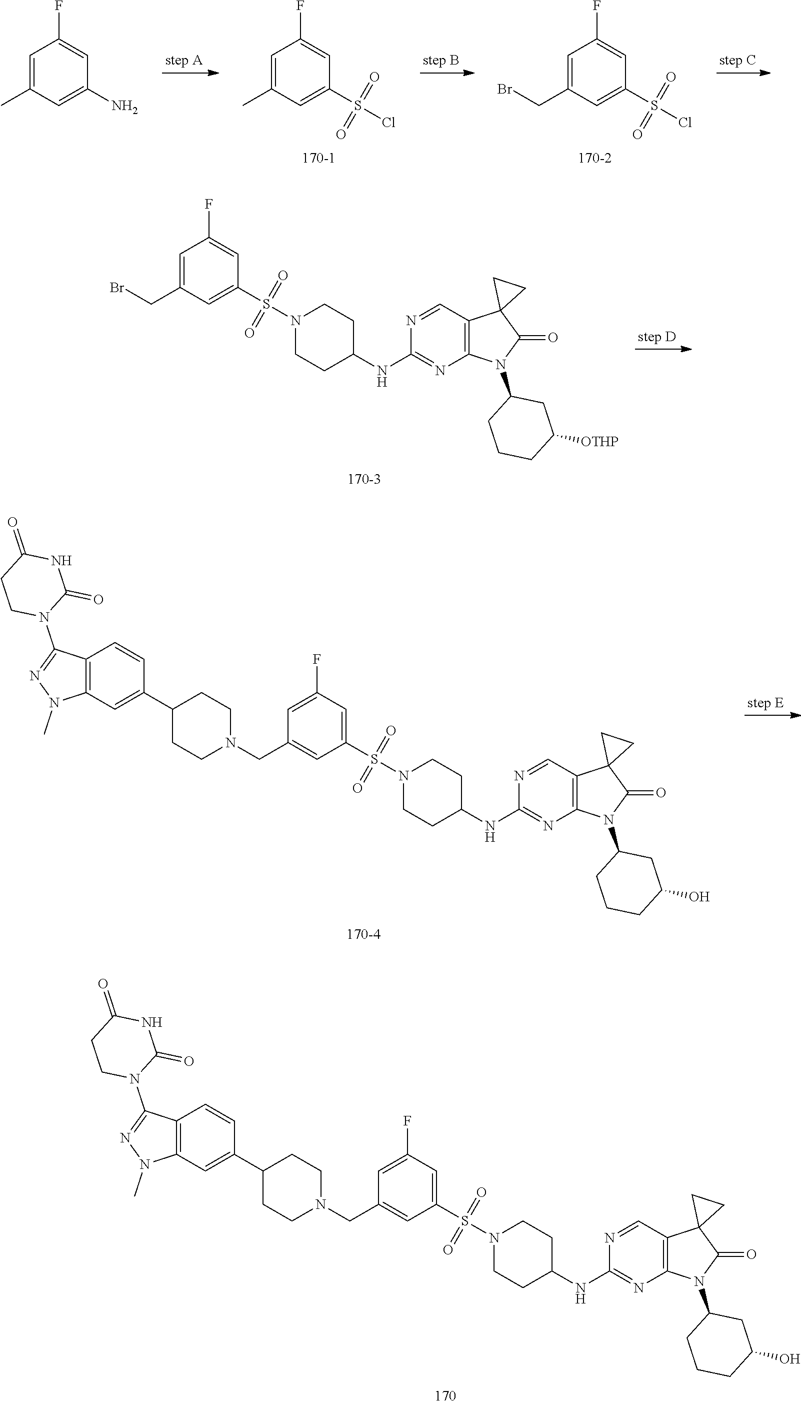

### Step A. 3-Fluoro-5-methylbenzenesulfonyl chloride (170-1)

At 0° C., HCl (6M) (27 mL) are added to the container of 3-fluoro-5-methylaniline (920 mg, 7.351 mmol, 1 equiv) and H₂O (102 mL) to begin stirring, and then NaNO₂ (567 mg, 8.218 mmol, 1.12 equiv) dissolved in H₂O (102 mL) is dropped into the solution. Reaction for 30 minutes, this reaction liquid is A. Add CuCl (76.8 mg, 0.776 mmol, 0.11 equiv) to H₂O (102 mL) at 0° C. and stir well, then drop in SOCl₂ (8.4 mL, 115.804 mmol, 15.75 equiv), and react at room temperature for 1 hour. This solution is B. Then liquid B was slowly dropped into liquid A at 0° C. and reacted for 1 h. The reaction was monitored by LCMS. The resulting mixture was extracted with CH₂Cl₂ (2×200 mL). The combined organic layers were washed with water (2×200 mL), dried over anhydrous Na₂SO₄. After filtration, the filtrate was concentrated under reduced pressure to afford 170-1 as a yellow oil. The crude product was used for next step directly.

### Step B. 3-(Bromomethyl)-5-fluorobenzenesulfonyl chloride (170-2)

To a mixture of 170-1 (860 mg, 4.122 mmol, 1 equiv) and NBS (770.36 mg, 4.328 mmol, 1.05 equiv) in CCl₄ (8 mL) was added benzoyl benzenecarboperoxoate (99.85 mg, 0.412 mmol, 0.1 equiv). The resulting mixture was stirred for overnight at 80° C. under nitrogen atmosphere. Desired product could be detected by LCMS. The resulting mixture was diluted with water and extracted with CH₂Cl₂. The combined organic layers were washed with water (2×10 mL), dried over anhydrous Na₂SO₄. After filtration, the filtrate was concentrated under reduced pressure. The crude product was purified by Prep-HPLC to afford 170-2 (101 mg, 8.49%) as a brown oil.

### Step C-E. 1-[6-(1-{[3-Fluoro-5-(4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl)phenyl]methyl}piperidin-4-yl)-1-methylindazol-3-yl]-1,3-diazinane-2,4-dione (170)

Compound 170 was prepared from 170-2, 7b and Int 24 following the procedure for the synthesis of compound 159 in example 159. LCMS (ESI, m/z): [M+H]⁺=855.3.

### Example 174: 1-[6-(1-{[3-(4-{7′-[(1R,3R)-3-Hydroxycyclohexyl]-6′-oxospiro[cyclobutane-1,5′-pyrrolo[2,3-d]pyrimidin]-2′-ylamino}piperidin-1-ylsulfonyl)phenyl]methyl}piperidin-4-yl)-1-methylindazol-3-yl]-1,3-diazinane-2,4-dione (174)

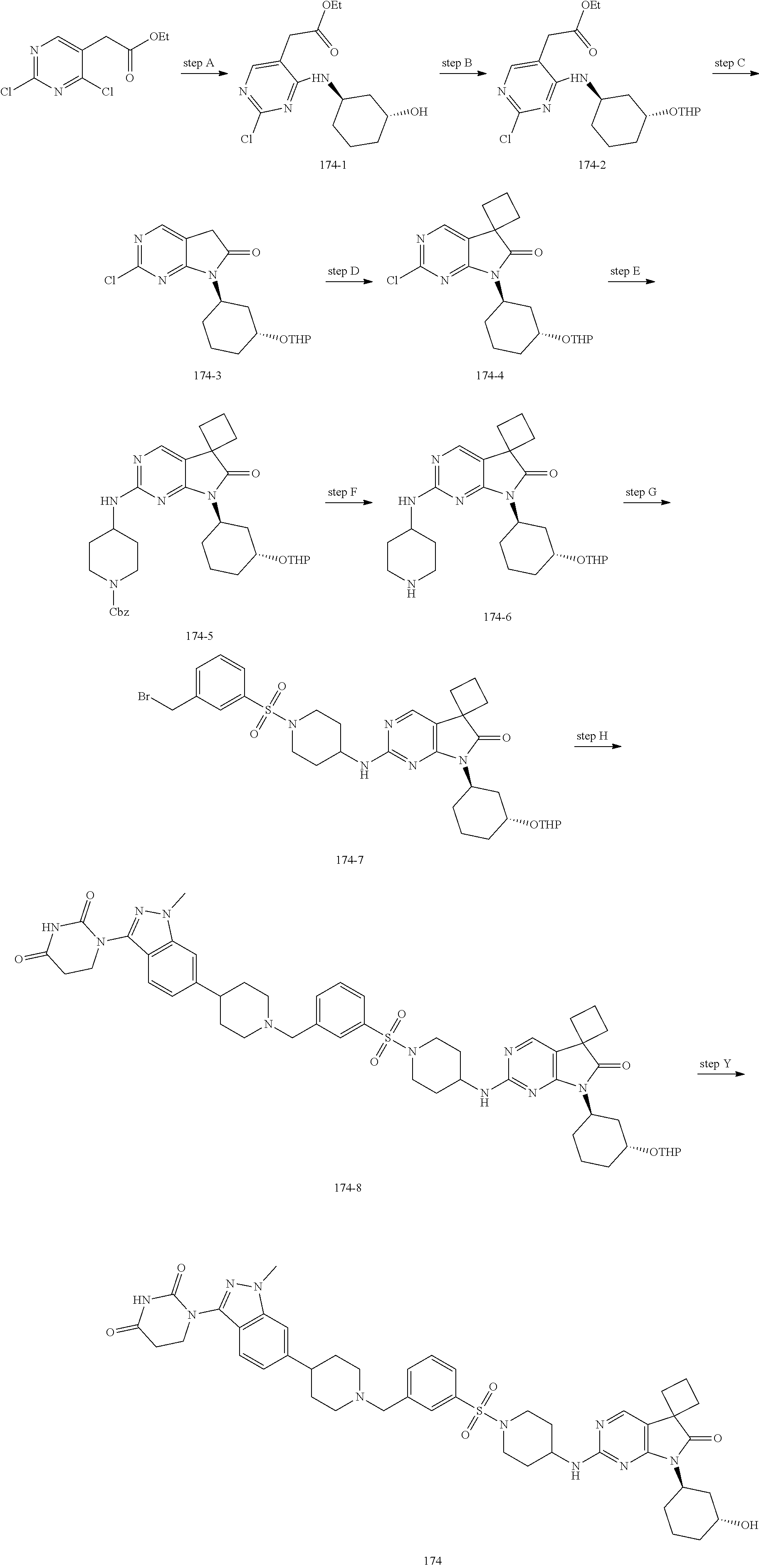

### Step A. Ethyl 2-(2-chloro-4-{[(1R,3R)-3-hydroxycyclohexyl]amino}pyrimidin-5-yl)acetate (174-1)

A solution of ethyl 2-(2,4-dichloropyrimidin-5-yl)acetate (1 g, 4.254 mmol, 1 equiv), DIEA (1649.54 mg, 12.762 mmol, 3 equiv) and (1R,3R)-3-aminocyclohexan-1-ol (489.99 mg, 4.254 mmol, 1.0 equiv) in MeCN (10 mL) was stirred for 3 h at 80° C. under argon atmosphere. Desired product could be detected by LCMS. The residue was purified by reversed-phase flash chromatography with the following conditions: column, C18 silica gel; mobile phase, MeCN in Water (0.1% FA), 10% to 60% gradient in 10 min; detector, UV 254 nm. to afford 174-1 (660 mg, 49.44%) as a yellow solid. LCMS (ESI, m/z): [M+H]⁺=314.

### Step B. Ethyl 2-(2-chloro-4-{[(1R,3R)-3-(oxan-2-yloxy)cyclohexyl]amino}pyrimidin-5-yl)acetate (174-2)

A solution of 174-1 (650 mg, 2.072 mmol, 1 equiv), TsOH (35.67 mg, 0.207 mmol, 0.1 equiv) and DHP (261.38 mg, 3.108 mmol, 1.5 equiv) in DCM (10 mL) was stirred for 3 h at 80° C. under argon atmosphere. Desired product could be detected by LCMS. The mixture was concentrated under vacuum. The residue was purified by silica gel column chromatography, eluted with PE/EA (1:1) to afford 174-2 (400 mg, 48.53%) as a yellow solid. LCMS (ESI, m/z): [M+H]⁺=398.

### Step C. 2-Chloro-7-[(1R,3R)-3-(oxan-2-yloxy)cyclohexyl]-5H-pyrrolo[2,3-d]pyrimidin-6-one (174-3)

To a stirred solution of 174-2 (2 g, 4.695 mmol, 1 equiv) in THF (20 mL) was added NaH (0.75 g, 18.780 mmol, 4 equiv, 60%). The resulting mixture was stirred for 0.5 hours at 25 degrees C. under nitrogen atmosphere. The reaction was monitored by LCMS. Desired product could be detected by LCMS. The resulting mixture was diluted with saturated aqueous NH₄C1 (20 mL). The resulting mixture was extracted with EtOAc (2×25 mL). The combined organic layers were washed with brine (2×15 mL), dried over anhydrous sodium sulfate. After filtration, the filtrate was concentrated under reduced pressure. The residue was purified by silica gel column chromatography, eluted with 0% to 15% EtOAc in PE. Pure fractions were evaporated to dryness to afford 174-3 (800 mg, 43.58%) as a yellow oil. LCMS (ESI, m/z): [M+H]⁺=352.

### Step D. 2′-Chloro-7′-[(1R,3R)-3-(oxan-2-yloxy)cyclohexyl]spiro[cyclobutane-1,5′-pyrrolo[2,3-d]pyrimidin]-6′-one (174-4)

A solution of 174-3 (550 mg, 1.563 mmol, 1 equiv), NaH (187.56 mg, 4.689 mmol, 3 equiv) and 1,3-dibromopropane (473.41 mg, 2.345 mmol, 1.5 equiv) in DMF (6 mL) was stirred for 30 min at 0° C. under argon atmosphere. Desired product could be detected by LCMS. The resulting mixture was diluted with saturated aqueous NH₄C1 (20 mL). The resulting mixture was extracted with EtOAc (2×50 mL). The combined organic layers were washed with brine (2×15 mL), dried over anhydrous sodium sulfate. After filtration, the filtrate was concentrated under reduced pressure. The residue was purified by silica gel column chromatography, eluted with PE/EA (2:1) to afford 174-4 (200 mg, 32.65%) as a yellow solid. LCMS (ESI, m/z): [M+H]⁺=314.

### Step E-F. 7′-[(1R,3R)-3-(oxan-2-yloxy)cyclohexyl]-2′-(piperidin-4-ylamino)spiro[cyclobutane-1,5′-pyrrolo[2,3-d]pyrimidin]-6′-one (174-6)

Compound 174-6 was prepared from 174-4 following the procedure for the synthesis of Int-7. LCMS (ESI, m/z): [M+H]⁺=416.

### Step G-H. 1-[6-(1-{[3-(4-{7′-[(1R,3R)-3-Hydroxycyclohexyl]-6′-oxospiro[cyclobutane-1,5′-pyrrolo[2,3-d]pyrimidin]-2′-ylamino}piperidin-1-ylsulfonyl)phenyl]methyl}piperidin-4-yl)-1-methylindazol-3-yl]-1,3-diazinane-2,4-dione (174)

Compound 174 was prepared from 174-6 and Int-24 following the procedure for the synthesis of compound 159 in example 159. LCMS (ESI, m/z): [M+H]⁺=851.4

### Example 175: 1-[7-fluoro-6-(1-{[3-(4-{7′-[(1R,3R)-3-Hydroxycyclohexyl]-6′-oxospiro[cyclopropane-1,5′-pyrrolo[2,3-d]pyrimidin]-2′-ylamino}piperidin-1-ylsulfonyl)phenyl]methyl}piperidin-4-yl)-1-methylindazol-3-yl]-1,3-diazinane-2,4-dione (175)

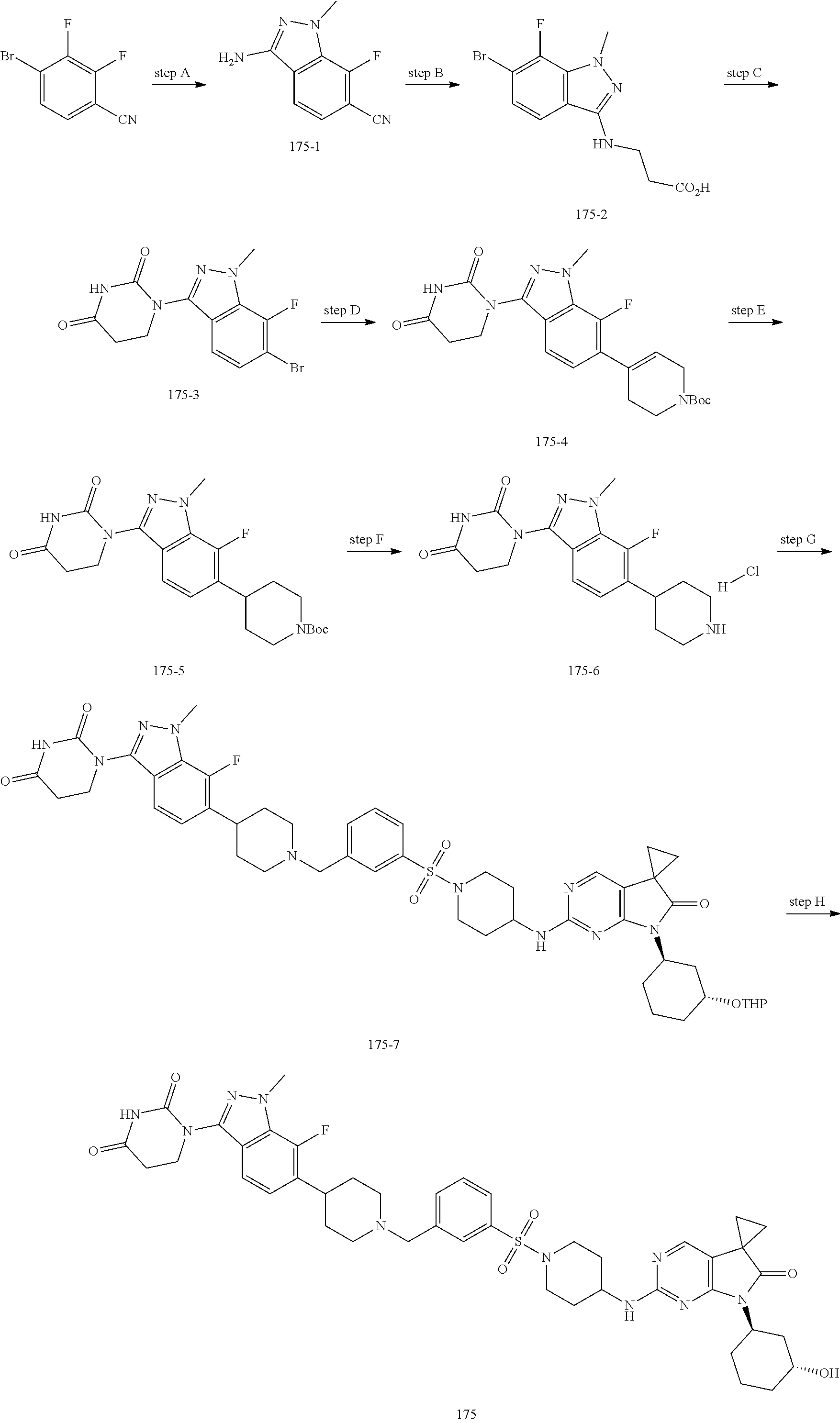

### Step A. 6-Bromo-7-fluoro-1-methylindazol-3-amine (175-1)

To a mixture of 4-bromo-2,3-difluorobenzonitrile (3 g, 13.761 mmol, 1 equiv) and methyl hydrazine (2.54 g, 55.044 mmol, 4 equiv) in EtOH (30 mL) was added DIPEA (17.79 g, 137.610 mmol, 10 equiv). The resulting mixture was stirred for 12 h at 120° C. The resulting mixture was concentrated under reduced pressure. The reaction was quenched by the addition of water. The resulting mixture was extracted with EtOAc. The combined organic layers were dried over anhydrous Na₂SO₄. After filtration, the filtrate was concentrated under reduced pressure. The residue was purified by silica gel column chromatography, eluted with PE/EA (1:1) to afford 175-1 (2 g, 59.19%) as a colorless oil. LCMS (ESI, m/z): [M+H]⁺=244.

### Step B. 3-[(6-Bromo-7-fluoro-1-methylindazol-3-yl)amino]propanoic acid (175-2)

To a mixture of 175-1 (1.5 g, 6.146 mmol, 1 equiv) and acrylic acid (1328.67 mg, 18.438 mmol, 3 equiv) in H₂O (20 mL) was added AcOH (1845.35 mg, 30.730 mmol, 5 equiv). The resulting mixture was stirred for 12 h at 100° C. Desired product could be detected by LCMS. The resulting mixture was filtered, the filter cake was washed with H₂O. The filtrate was concentrated under reduced pressure. The residue was purified by reversed-phase Combi-Flash, eluted with CH₃CN:H₂O (0.5% NH₄HCO₃)=5%-30%. This resulted in 175-2 (900 mg, 42.29%) as a brown oil. LCMS (ESI, m/z): [M+H]⁺=316.

### Step C. 1-(6-Bromo-7-fluoro-1-methylindazol-3-yl)-1,3-diazinane-2,4-dione (175-3)

To a stirred mixture of 175-2 (940 mg, 2.731 mmol, 1 equiv) in AcOH (10 mL) was added sodium cyanate (355.08 mg, 5.462 mmol, 2 equiv). The resulting mixture was stirred for 12 h at 65° C. The resulting mixture was added 4M HCl (9.4 mL, 37.600 mmol, 13.77 equiv). The resulting mixture was stirred for 4 h at 65° C. The resulting mixture was filtered, the filter cake was washed with H₂O. The filtrate was concentrated under reduced pressure to afford 175-3 (480 mg, 30.76%) as a brown solid. LCMS (ESI, m/z): [M+H]⁺=341.

### Step D. Tert-butyl 4-[3-(2,4-dioxo-1,3-diazinan-1-yl)-7-fluoro-1-methylindazol-6-yl]-3,6-dihydro-2H-pyridine-1-carboxylate (175-4)

To a stirred mixture of 175-3 (460 mg, 1.348 mmol, 1 equiv) and tert-butyl 4-(4,4,5,5-tetramethyl-1,3,2-dioxaborolan-2-yl)-3,6-dihydro-2H-pyridine-1-carboxylate (500.33 mg, 1.618 mmol, 1.2 equiv) in dioxane (5 mL) and H₂O (1 mL) was added NaOAc (276.54 mg, 3.370 mmol, 2.5 equiv) and Pd(dppf)Cl₂ (98.67 mg, 0.135 mmol, 0.1 equiv). The resulting mixture was stirred for 1 h at 100° C. Desired product could be detected by LCMS. The resulting mixture was concentrated under reduced pressure. The residue was purified by silica gel column chromatography, eluted with EA/PE (1:1) to afford 175-4 (436 mg, 67.44%) as a white solid. LCMS (ESI, m/z): [M+H]⁺=444.

### Step E-H. 1-[7-Fluoro-6-(1-{[3-(4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl)phenyl]m ethyl}piperidin-4-yl)-1-methylindazol-3-yl]-1,3-diazinane-2,4-dione (175)

Compound 175 was prepared from 175-4 and Int 7 following the procedure for the synthesis of compound 146 in example 146. LCMS (ESI, m/z): [M+H]⁺=855.3.

### Example 176: 1-[5-Fluoro-6-(1-{[3-(4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl)phenyl]methyl}piperidin-4-yl)-1-methylindazol-3-yl]-1,3-diazinane-2,4-dione (176)

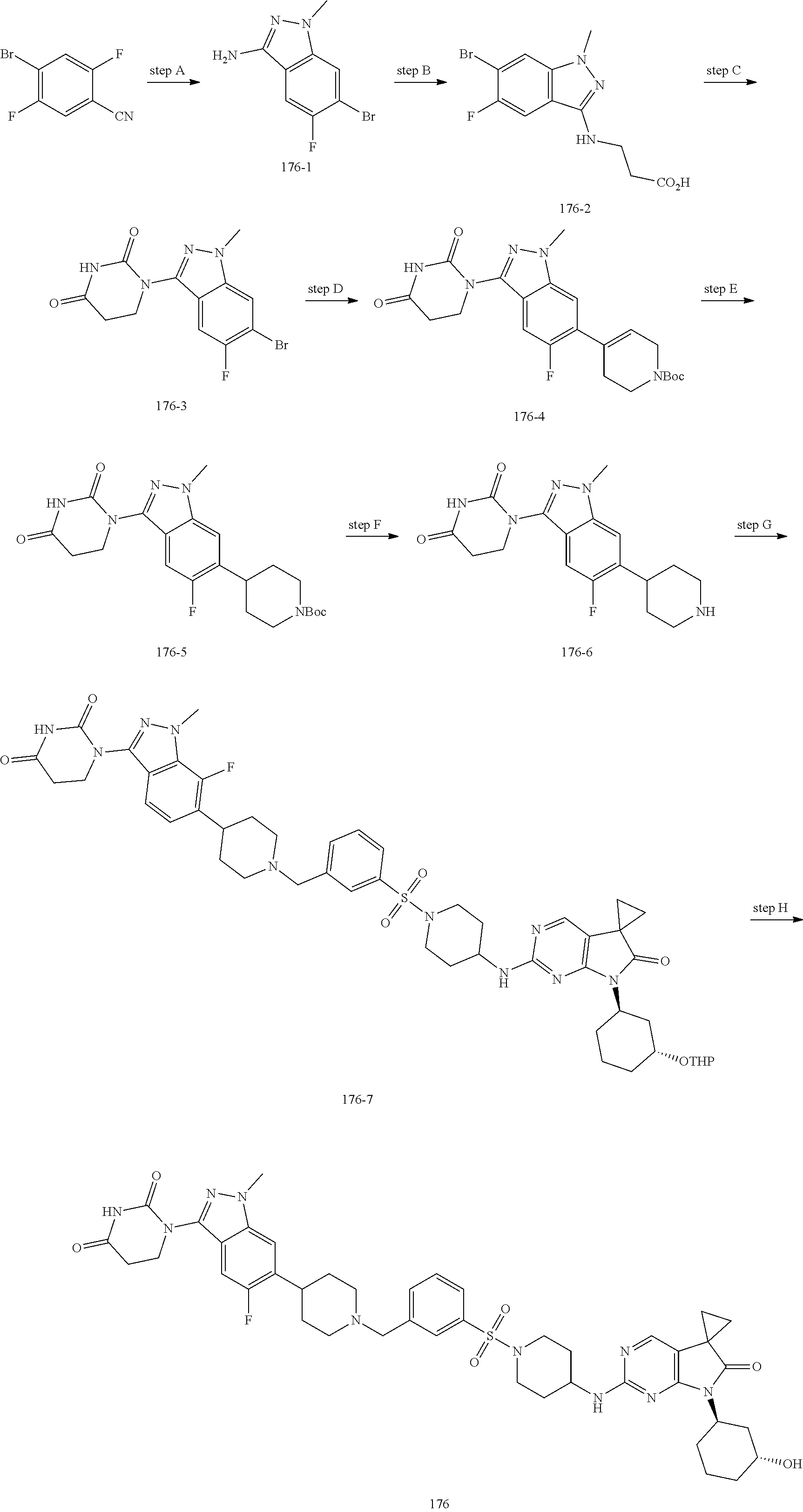

### Step A. 6-Bromo-5-fluoro-1-methylindazol-3-amine (176-1)

A solution of 4-bromo-2,5-difluorobenzonitrile (6 g, 27.523 mmol, 1 equiv) and methyl hydrazine (5072.23 mg, 110.092 mmol, 4 equiv) in EtOH (60 mL) was stirred for 2 h at 80° C. under argon atmosphere. Desired product could be detected by LCMS. The resulting mixture was concentrated under reduced pressure. The residue was purified by silica gel column chromatography, eluted with PE/EA (1:3) to afford 176-1 (4 g, 59.55%) as a yellow solid. LCMS (ESI, m/z): [M+H]⁺=244.

### Step B. 3-[(6-Bromo-5-fluoro-1-methylindazol-3-yl)amino]propanoic acid (176-2)

A solution of 176-1 (1 g, 4.097 mmol, 1 equiv), acrylic acid (442.89 mg, 6.146 mmol, 1.5 equiv) and TBAB (132.08 mg, 0.410 mmol, 0.1 equiv) in HCl (2M) (10 mL) was stirred for overnight at 100° C. under argon atmosphere. Desired product could be detected by LCMS. The mixture was neutralized to pH 7 with saturated NaHCO₃ (aq.). The precipitated solids were collected by filtration and washed with water (2×10 mL) to afford 176-2 (500 mg, 38.60%) as a brown solid. LCMS (ESI, m/z): [M+H]⁺=316.

### Step C. 1-(6-Bromo-5-fluoro-1-methylindazol-3-yl)-1,3-diazinane-2,4-dione (176-3)

A solution of 176-2 (300 mg, 0.949 mmol, 1 equiv) and urea (170.97 mg, 2.847 mmol, 3 equiv) in HOAc (5 mL) was stirred for overnight at 100° C. under argon atmosphere. Desired product could be detected by LCMS. The residue was purified by reversed-phase flash chromatography with the following conditions: column, C18 silica gel; mobile phase, MeCN in Water (10 mmol/L NH₄HCO₃), 10% to 60% gradient in 10 min; detector, UV 254 nm. to afford 176-3 (100 mg, 30.89%) as a yellow solid. LCMS (ESI, m/z): [M+H]⁺=341.

### Step D-H. 1-[5-Fluoro-6-(1-{[3-(4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl)phenyl]m ethyl}piperidin-4-yl)-1-methylindazol-3-yl]-1,3-diazinane-2,4-dione (176)

Compound 176 was prepared from 176-3 and Int 7 following the procedure for the synthesis of compound 175 in example 175. LCMS (ESI, m/z): [M+H]⁺=855.4.

### Example 177: 1-[6-(3,3-Difluoro-1-{[3-(4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl)phenyl]methyl}piperidin-4-yl)-1-methylindazol-3-yl]-1,3-diazinane-2,4-dione (177)

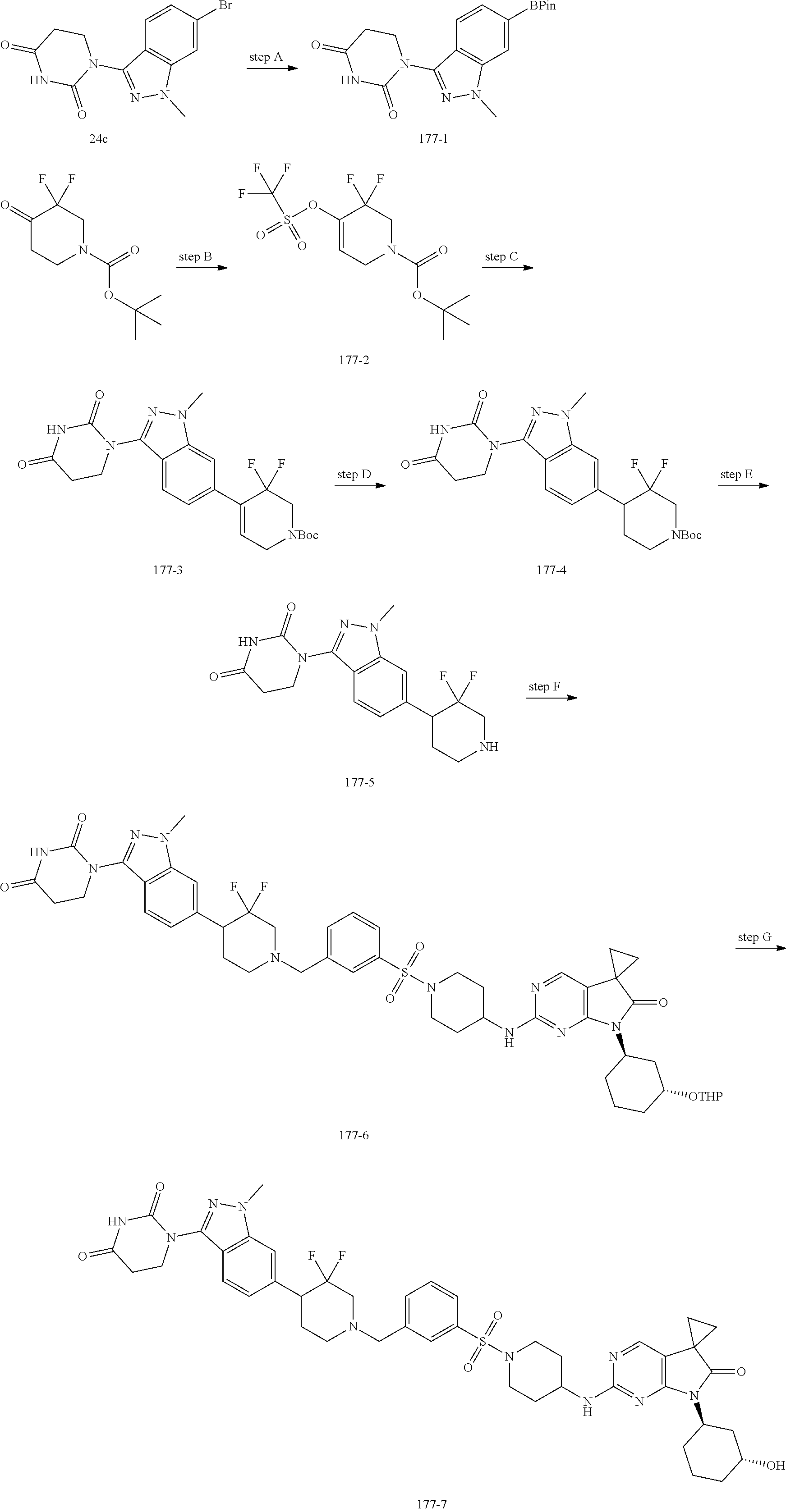

### Step A. 1-[1-Methyl-6-(4,4,5,5-tetramethyl-1,3,2-dioxaborolan-2-yl) indazol-3-yl]-1,3-diazinane-2,4-dione (177-1)

A solution of 24c (300 mg, 0.928 mmol, 1 equiv), 4,4,5,5-tetramethyl-2-(4,4,5,5-tetramethyl-1,3,2-dioxaborolan-2-yl)-1,3,2-dioxaborolane (471.49 mg, 1.856 mmol, 2 equiv), AcOK (182.22 mg, 1.856 mmol, 2 equiv) and Pd(dppf)Cl₂ (67.93 mg, 0.093 mmol, 0.1 equiv) in dioxane (5 mL) was stirred for 1 h at 80° C. under nitrogen atmosphere. Desired product could be detected by LCMS. The resulting mixture was diluted with water. The aqueous layer was extracted with EtOAc (3×100 mL). The residue was purified by silica gel column chromatography, eluted with PE/EA (5:1) to afford 177-1 (180 mg, 52.37%) as a white solid. LCMS (ESI, m/z): [M+H]⁺=371.

### Step B. Tert-butyl 3,3-difluoro-4-(trifluoromethanesulfonyloxy)-2,6-dihydropyridine-1-carboxylate (177-2)

To a stirred solution of tert-butyl 3,3-difluoro-4-oxopiperidine-1-carboxylate (2 g, 8.502 mmol, 1 equiv) in DCM (20 mL) was added Et₃N (2.58 g, 25.506 mmol, 3 equiv) and Tf₂O (3.60 g, 12.760 mmol, 1.50 equiv) dropwise at 0° C. The resulting mixture was stirred for overnight at room temperature. Desired product could be detected by LCMS. The reaction was quenched by the addition of water. The resulting mixture was extracted with CH₂Cl₂. The combined organic layers were dried over anhydrous Na₂SO₄. After filtration, the filtrate was concentrated under reduced pressure. The residue was purified by silica gel column chromatography, eluted with PE/EA (10:1) to afford 177-2 (440 mg, 14.09%) as a yellow oil. LCMS (ESI, m/z): [M+H]⁺=368.

### Step C. Tert-butyl 4-[3-(2,4-dioxo-1,3-diazinan-1-yl)-1-methylindazol-6-yl]-3,3-difluoro-2,6-dihydropyridine-1-carboxylate (177-3)

A solution of 177-1 (200 mg, 0.540 mmol, 1 equiv) and 177-2 (238.10 mg, 0.648 mmol, 1.2 equiv), Pd(dppf)Cl₂ (39.53 mg, 0.054 mmol, 0.1 equiv) and Na₂CO₃ (171.77 mg, 1.620 mmol, 3 equiv) in dioxane (4 mL)/H₂O (0.8 mL) was stirred for 2 h at 60° C. under argon atmosphere. Desired product could be detected by LCMS. The resulting mixture was diluted with water. The resulting mixture was extracted with EtOAc. The combined organic layers were dried over anhydrous Na₂SO₄. After filtration, the filtrate was concentrated under reduced pressure. The residue was purified by Prep-TLC (CH₂Cl₂/MeOH 20:1) to afford 177-3 (200 mg, 80.23%) as a white semi-solid. LCMS (ESI, m/z): [M+H]⁺=462.

### Step D. Tert-butyl 4-[3-(2,4-dioxo-1,3-diazinan-1-yl)-1-methylindazol-6-yl]-3,3-di fluoropiperidine-1-carboxylate (177-4)

A solution of 177-3 (180 mg, 0.390 mmol, 1 equiv) and Pd/C (415 mg) in dioxane (5 mL) was stirred for overnight at room temperature under hydrogen atmosphere. Desired product could be detected by LCMS. The resulting mixture was filtered, the filter cake was washed with MeOH (200 mL). The filtrate was concentrated under reduced pressure. The residue was purified by silica gel column chromatography, eluted with PE/EA (1:3) to afford 177-4 (135 mg, 67.21%) as an off-white solid. LCMS (ESI, m/z): [M+H]⁺=464.

### Step E. 1-(6-(3,3-Difluoropiperidin-4-yl)-1-methyl-1H-indazol-3-yl)dihydropyrimidine-2,4(1H,3H)-dione (177-5)

To a stirred mixture of 177-4 (120 mg, 0.259 mmol, 1 equiv) in DCM (2 mL) was added TFA (0.4 mL) dropwise at 0° C. under nitrogen atmosphere. The resulting mixture was stirred for 1 h at room temperature under nitrogen atmosphere. Desired product could be detected by LCMS. The resulting mixture was concentrated under reduced pressure. The crude product was used in the next step directly without further purification. LCMS (ESI, m/z): [M+H]⁺=364.

### Step F-G. 1-[6-(3,3-difluoro-1-{[3-(4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl)phenyl]methyl}piperidin-4-yl)-1-methylindazol-3-yl]-1,3-diazinane-2,4-dione (177)

Compound 177 was prepared from 177-5 and Int 7 following the procedure for the synthesis of compound 121 in example 121. LCMS (ESI, m/z): [M+H]⁺=873.4.

### Example 179: 3-[5-(4-{[3-(4-{7′-[(1R,3R)-3-Hydroxycyclohexyl]-6′-oxospiro[cyclopropane-1,5′-pyrrolo[2,3-d]pyrimidin]-2′-ylamino}piperidin-1-ylsulfonyl)phenyl]methyl}piperazin-1-yl)-3-methyl-2-oxo-1,3-benzodiazol-1-yl]piperidine-2,6-dione (179)

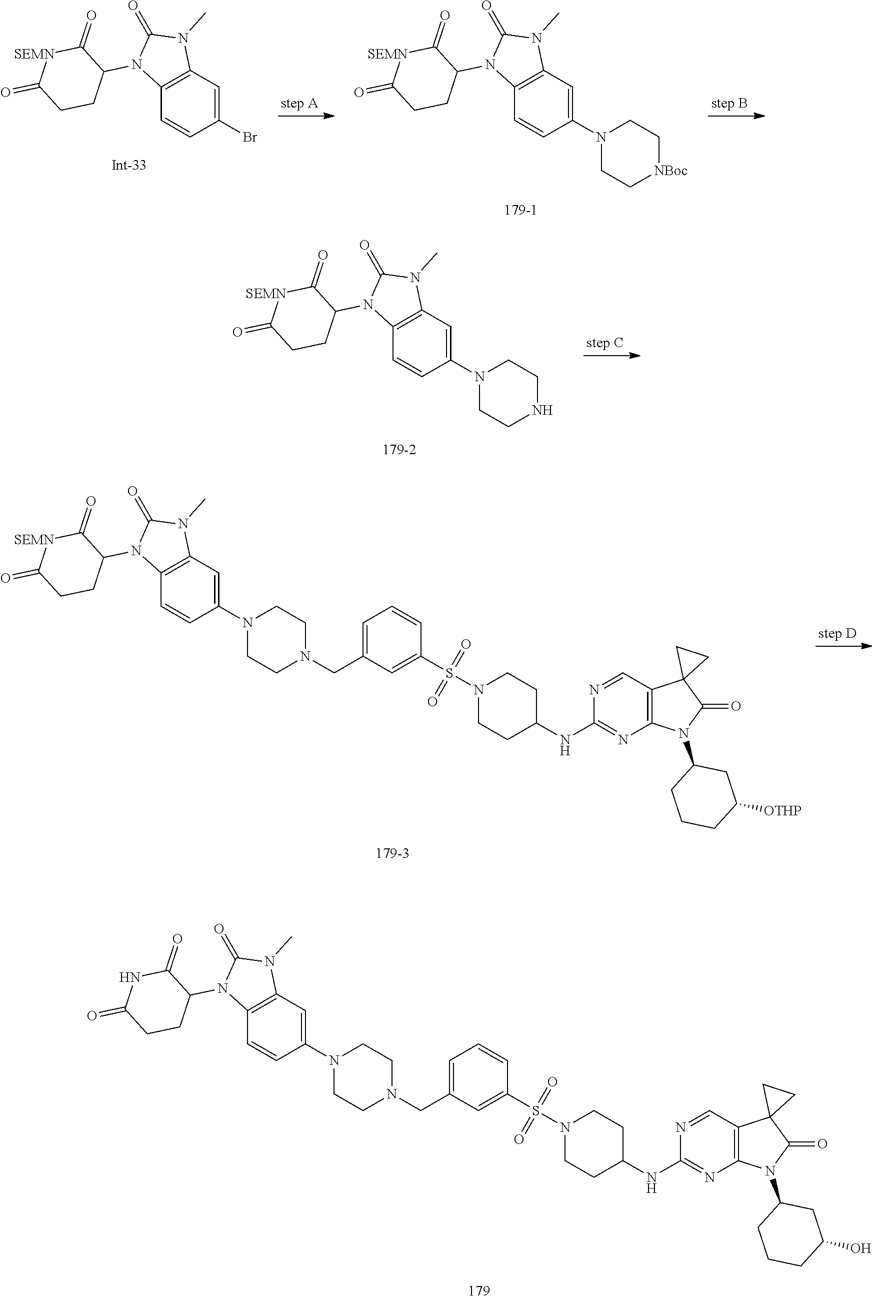

### Step A. Tert-butyl 4-[1-(2,6-dioxo-1-{[2-(trimethylsilyl)ethoxy]methyl}piperidin-3-yl)-3-methyl-2-oxo-1,3-benzodiazol-5-yl]piperazine-1-carboxylate (179-1)

To a stirred mixture of Int-33 (400 mg, 0.854 mmol, 1 equiv) and tert-butyl piperazine-1-carboxylate (318.10 mg, 1.708 mmol, 2 equiv) in dioxane (5 mL) were added RuphosPdG3 (71.51 mg, 0.085 mmol, 0.1 equiv), Ruphos (79.70 mg, 0.171 mmol, 0.2 equiv) and Cs₂CO₃ (834.68 mg, 2.562 mmol, 3 equiv) at room temperature under argon atmosphere. The resulting mixture was stirred for 1 h at 100° C. under argon atmosphere. The resulting mixture was concentrated under reduced pressure. The residue was purified by silica gel column chromatography, eluted with PE/EA (1:1) to afford 179-1 (300 mg, 61.23%) as a white solid. LCMS (ESI, m/z): [M+H]⁺=574.

### Step B. 3-[3-Methyl-2-oxo-5-(piperazin-1-yl)-1,3-benzodiazol-1-yl]-1-{[2-(trimethylsilyl)ethoxy]methyl}piperidine-2,6-dione (179-2)

To a stirred mixture of 179-1 (100 mg, 0.174 mmol, 1 equiv) in EA (2 mL) was added TsOH (120.05 mg, 0.696 mmol, 4 equiv) at 0° C. The resulting mixture was stirred for 1 h at 0° C. Desired product could be detected by LCMS. The reaction was quenched by the addition of NaHCO₃ (5 mL). The resulting mixture was extracted with EA. The combined organic layers were dried over anhydrous Na₂SO₄. After filtration, the filtrate was concentrated under reduced pressure to afford 179-2 (100 mg, crude) as a white solid. LCMS (ESI, m/z): [M+H]⁺=474.

### Step C. 3-[3-Methyl-5-(4-{[3-(4-{7′-[(1R,3R)-3-(oxan-2-yloxy)cyclohexyl]-6′-oxospiro[cyclopropane-1,5′-pyrrolo[2,3-d]pyrimidin]-2′-ylamino}piperidin-1-ylsulfonyl)phenyl]methyl}piperazin-1-yl)-2-oxo-1,3-benzodiazol-1-yl]-1-{[2-(trimethylsilyl)ethoxy]methyl}piperidine-2,6-dione (179-3)

Compound 179-3 was prepared from 179-2 following the procedure for the synthesis of compound 159-4 in example 159. LCMS (ESI, m/z): [M+H]⁺=1067.

### Step D. 3-[5-(4-{[3-(4-{7′-[(1R,3R)-3-Hydroxycyclohexyl]-6′-oxospiro[cyclopropane-1,5′-pyrrolo[2,3-d]pyrimidin]-2′-ylamino}piperidin-1-ylsulfonyl)phenyl]methyl}piperazin-1-yl)-3-methyl-2-oxo-1,3-benzodiazol-1-yl]piperidine-2,6-dione (179)

Compound 179 was prepared from 179-3 following the procedure for the synthesis of compound 135 in example 135. LCMS (ESI, m/z): [M+H]⁺=853.6.

### Example 180: 1-[6-(3-Fluoro-1-{[3-(4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl)phenyl]methyl}piperidin-4-yl)-1-methylindazol-3-yl]-1,3-diazinane-2,4-dione (180)

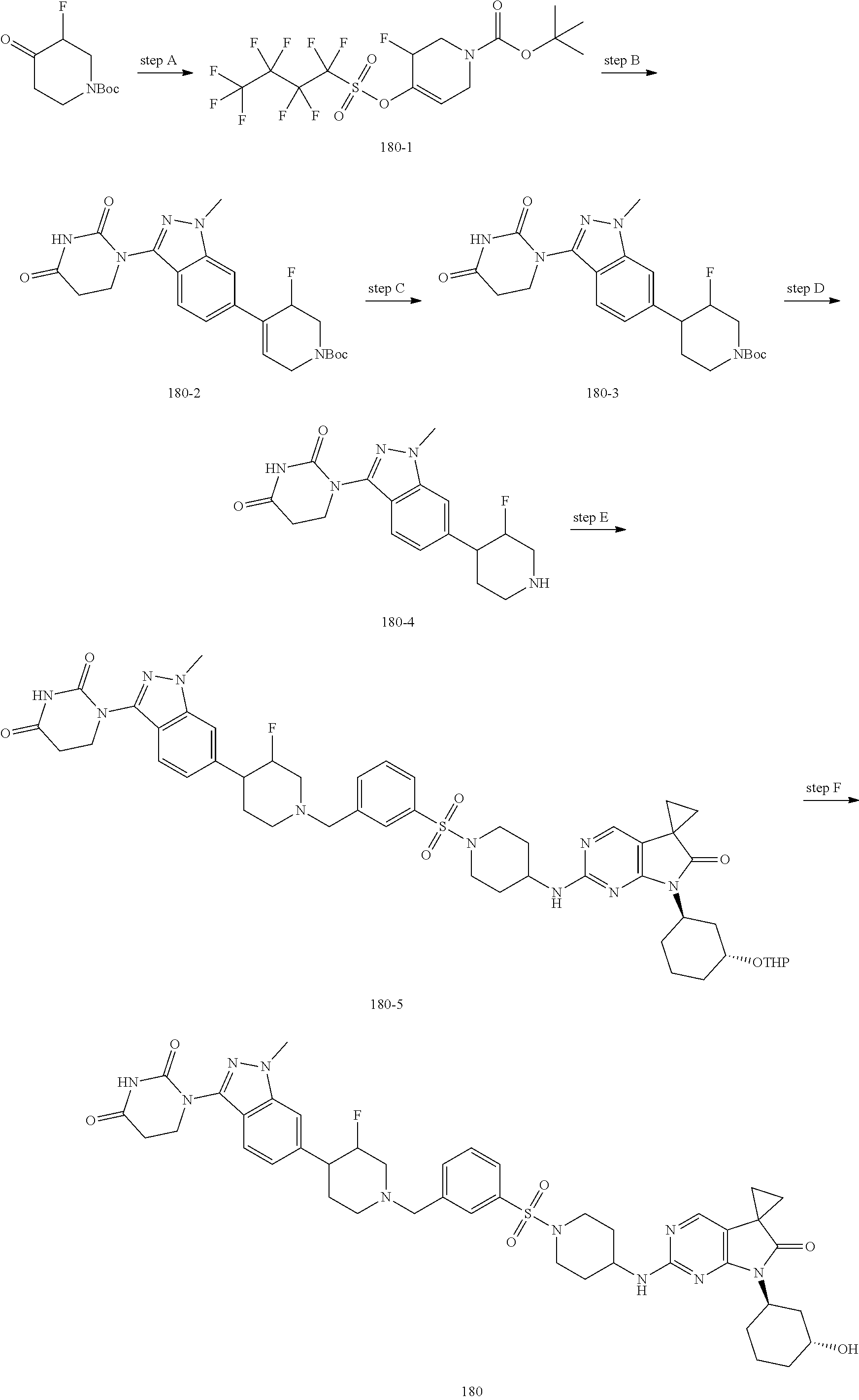

### Step A. Tert-butyl 3-fluoro-4-[(1,1,2,2,3,3,4,4,4-nonafluorobutanesulfonyl)oxy]-3, 6-dihydro-2H-pyridine-1-carboxylate (180-1)

To a solution of tert-butyl 3-fluoro-4-oxopiperidine-1-carboxylate (2.1 g, 9.667 mmol, 1 equiv) in THF (30 mL) was added DBU (2.94 g, 19.334 mmol, 2 equiv) and perfluorobutanesulfonyl fluoride (8.76 g, 29.001 mmol, 3 equiv) at 0° C. The mixture was stirred for 2 hours at −20° C. The resulting mixture was concentrated under reduced pressure. The residue was purified by silica gel column chromatography, eluted with PE/EA (5/1) to afford 180-1 (650 mg, 13.47%) as a white solid. LCMS (ESI, m/z): [M+H]⁺=500.

### Step B-F. 1-[6-(3-Fluoro-1-{[3-(4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl)phenyl]m ethyl}piperidin-4-yl)-1-methylindazol-3-yl]-1,3-diazinane-2,4-dione (180)

Compound 180 was prepared from 180-1 and Int 7 following the procedure for the synthesis of compound 177 in example 177. LCMS (ESI, m/z): [M+H]⁺=855.4.

### Example 181: 3-[4-(4-{[3-(4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl)phenyl]methyl}piperazin-1-yl)-3-methyl-2-oxo-1,3-benzodiazol-1-yl]piperidine-2,6-dione (181)

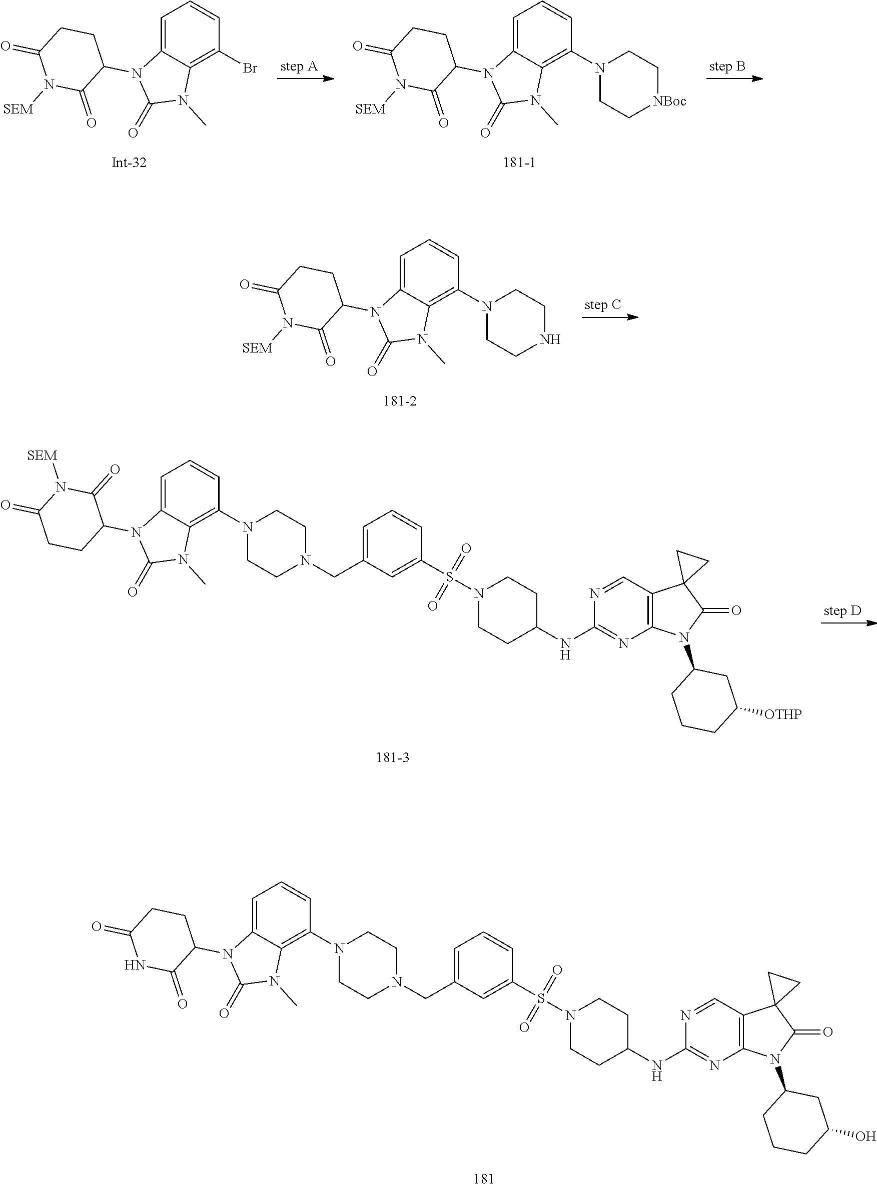

### Step A. Tert-butyl 4-[1-(2,6-dioxo-1-{[2-(trimethylsilyl)ethoxy]methyl}piperidin-3-yl)-3-methyl-2-oxo-1,3-benzodiazol-4-yl]piperazine-1-carboxylate (181-1)

To a stirred solution of Int-32 (800 mg, 1.708 mmol, 1 equiv), tert-butyl piperazine-1-carboxylate (636.19 mg, 3.416 mmol, 2 equiv) and RuPhos (159.39 mg, 0.342 mmol, 0.2 equiv) in dioxane (10 mL) were added Pd₂(dba)₃ (156.39 mg, 0.171 mmol, 0.1 equiv) and Cs₂CO₃ (1.11 g, 3.416 mmol, 2 equiv) at room temperature under nitrogen atmosphere. The resulting mixture was stirred for 1 h at 100° C. under nitrogen atmosphere. The reaction was monitored by LCMS. The resulting mixture was concentrated under reduced pressure. The residue was purified by reversed-phase flash chromatography with the following conditions: column, C18 silica gel; mobile phase, MeCN in Water (10 mmol/L NH₄HCO₃), 10% to 60% gradient in 10 min; detector, UV 254 nm. This resulted in 181-1 (400 mg, 40.82%) as a yellow solid. LCMS (ESI, m/z): [M+H]⁺=574.

### Step B. 3-[3-Methyl-2-oxo-4-(piperazin-1-yl)-1,3-benzodiazol-1-yl]-1-{[2-(trimethylsilyl)ethoxy]methyl}piperidine-2,6-dione (181-2)

A solution of 181-1 (300 mg, 0.523 mmol, 1 equiv) and 4M HCl (gas) in 1,4-dioxane (1.5 mL) in ACN (3 mL) was stirred for 1 h at 0° C. under nitrogen atmosphere. The reaction w as monitored by LCMS. The resulting mixture was concentrated under reduced pressure. This resulted in 181-2 (200 mg, 80.76%) as a white solid. The crude product was used directly for next step. LCMS (ESI, m/z): [M+H]⁺=474.

### Step C-D. 3-[4-(4-{[3-(4-{7′-[(1R,3R)-3-Hydroxycyclohexyl]-6′-oxospiro[cyclopropane-1,5′-pyrrolo[2,3-d]pyrimidin]-2′-ylamino}piperidin-1-ylsulfonyl)phenyl]methyl}piperazin-1-yl)-3-methyl-2-oxo-1,3-benzodiazol-1-yl]piperidine-2,6-dione (181)

Compound 181 was prepared from 181-2 and Int 7 following the procedure for the synthesis of compound 178 in example 178. LCMS (ESI, m/z): [M+H]⁺=853.4.

### Example 182: 1-[6-(1-{[3-(4-{7′-[(1R,3R)-3-Hydroxycyclohexyl]-6′-oxospiro[cyclopropane-1,5′-pyrrolo[2,3-d]pyrimidin]-2′-ylamino}piperidin-1-ylsulfonyl)phenyl]methyl}-3-methylpiperidin-4-yl)-1-methylindazol-3-yl]-1,3-diazinane-2,4-dione (182)

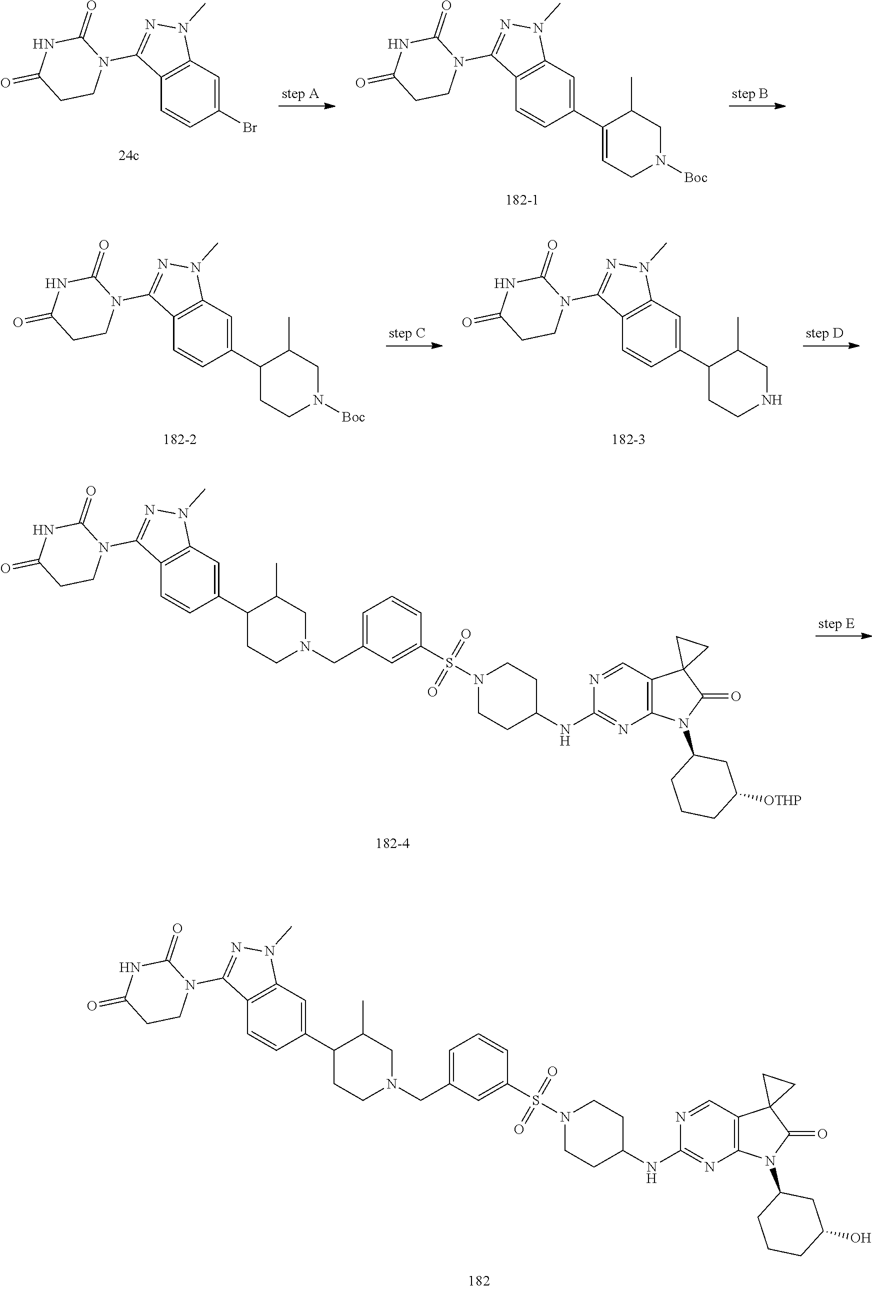

### Step A. Tert-butyl 4-[3-(2,4-dioxo-1,3-diazinan-1-yl)-1-methylindazol-6-yl]-3-methyl-3,6-dihydro-2H-pyridine-1-carboxylate (182-1)

To a stirred solution of 24c (480 mg, 1.485 mmol, 1 equiv) and tert-butyl 3-methyl-4-(4,4,5,5-tetramethyl-1,3,2-dioxaborolan-2-yl)-3,6-dihydro-2H-pyridine-1-carboxylate (480.13 mg, 1.485 mmol, 1 equiv) in dioxane (15 mL)/H₂O (4 mL) were added XPhos Pd G3 (3.93 mg, 0.005 mmol, 0.1 equiv) and K₃PO₄ (945.88 mg, 4.455 mmol, 3 equiv) at room temperature under nitrogen atmosphere. The resulting mixture was stirred for 1 h at 80° C. under nitrogen atmosphere. The mixture was allowed to cool down to room temperature. The resulting mixture was diluted with brine. The aqueous layer was extracted with EtOAc. The combined organic layers were concentrated under reduced pressure. The residue was purified by reversed-phase flash chromatography with the following conditions: column, C18 silica gel; mobile phase, MeCN in Water (0.1% FA), 5% to 48% gradient in 45 min; detector, UV 254 nm. This resulted in 182-1 (450 mg, 68.93%) as a white solid. LCMS (ESI, m/z): [M+H]⁺=440.

### Step B. Tert-butyl 4-[3-(2,4-dioxo-1,3-diazinan-1-yl)-1-methylindazol-6-yl]-3-methylpiperidine-1-carboxylate (182-2)

To a solution of 182-1 (400 mg, 0.910 mmol, 1 equiv) in THF (10 mL) were added Pd/C (193 mg). The resulting mixture was stirred for 5 h at 25 degrees C. under H₂ atmosphere. Desired product could be detected by LCMS. After filtration, the filtrate was concentrated under reduced pressure to afford 300 mg crude product and the crude product was used for next step directly. LCMS (ESI, m/z): [M+H]⁺=442.

### Step C-E. 3-[4-(4-{[3-(4-{7′-[(1R,3R)-3-Hydroxycyclohexyl]-6′-oxospiro[cyclopropane-1,5′-pyrrolo[2,3-d]pyrimidin]-2′-ylamino}piperidin-1-ylsulfonyl)phenyl]methyl}piperazin-1-yl)-3-methyl-2-oxo-1,3-benzodiazol-1-yl]piperidine-2,6-dione (182)

Compound 182 was prepared from 182-2 and Int 7 following the procedure for the synthesis of compound 159 in example 159. LCMS (ESI, m/z): [M+H]⁺=851.3.

### Example 183: 1-[6-(1-{[3-(4-{7′-[(1R,3R)-3-Hydroxycyclohexyl]-6′-oxospiro[cyclopropane-1,5′-pyrrolo[2,3-d]pyrimidin]-2′-ylamino}piperidin-1-ylsulfonyl)phenyl]methyl}-2,2-dimethylpiperidin-4-yl)-1-methylindazol-3-yl]-1,3-diazinane-2,4-dione (183)

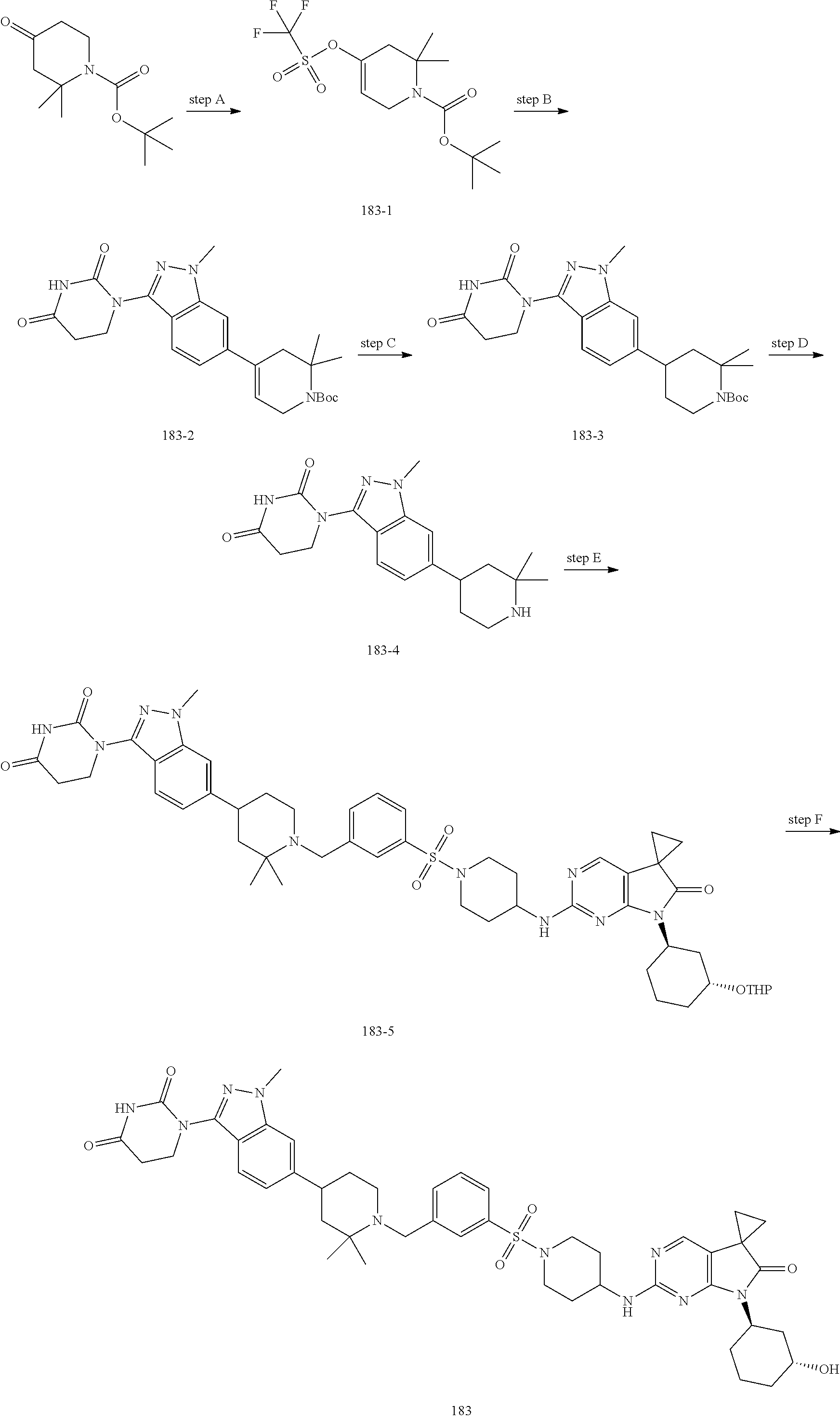

### Step A. Tert-butyl 2,2-dimethyl-4-(trifluoromethanesulfonyloxy)-3,6-dihydropyridine-1-carboxylate (183-1)

To a stirred solution of tert-butyl 2,2-dimethyl-4-oxopiperidine-1-carboxylate (1 g, 4.399 mmol, 1 equiv) in THF (9 mL) was added LiHMDS (1M in THF) (4.84 mL, 4.839 mmol, 1.1 equiv) dropwise at −78° C. under argon atmosphere. The resulting mixture was stirred for 20 min at −78° C. under argon atmosphere. Then 1,1,1-trifluoro-N-phenyl-N-trifluoromethanesulfonylmethanesulfonamide (1.73 g, 4.839 mmol, 1.1 equiv) was added. The resulting mixture was stirred for overnight at room temperature under argon atmosphere. The reaction was quenched with sat. NH₄Cl (aq.) at room temperature. The resulting mixture was extracted with CH₂Cl₂. The combined organic layers were dried over anhydrous Na₂SO₄. After filtration, the filtrate was concentrated under reduced pressure. The residue was purified by silica gel column chromatography, eluted with PE/EA (8:1) to afford 183-1 (1 g, 63.25%) as a yellow oil. LCMS (ESI, m/z): [M+H]⁺=360.

### Step B-F. 1-[6-(1-{[3-(4-{7′-[(1R,3R)-3-Hydroxycyclohexyl]-6′-oxospiro[cyclopropane-1,5′-pyrrolo[2,3-d]pyrimidin]-2′-ylamino}piperidin-1-ylsulfonyl)phenyl]methyl}-2, 2-dimethylpiperidin-4-yl)-1-methylindazol-3-yl]-1,3-diazinane-2,4-dione (183)

Compound 183 was prepared from 183-1 and Int 7 following the procedure for the synthesis of compound 177 in example 177. LCMS (ESI, m/z): [M+H]⁺=865.3.

### Example 185: 7′-((1R,3R)-3-hydroxycyclohexyl)-2′-((1-((3-((3-methyl-4-(1-methyl-3-(2-oxotetrahydropyrimidin-1(2H)-yl)-1H-indazol-6-yl)piperazin-1-yl)methyl)phenyl)sulfonyl)piperidin-4-yl)amino)spiro[cyclopropane-1,5′-pyrrolo[2,3-d]pyrimidin]-6′(7′H)-one (185)

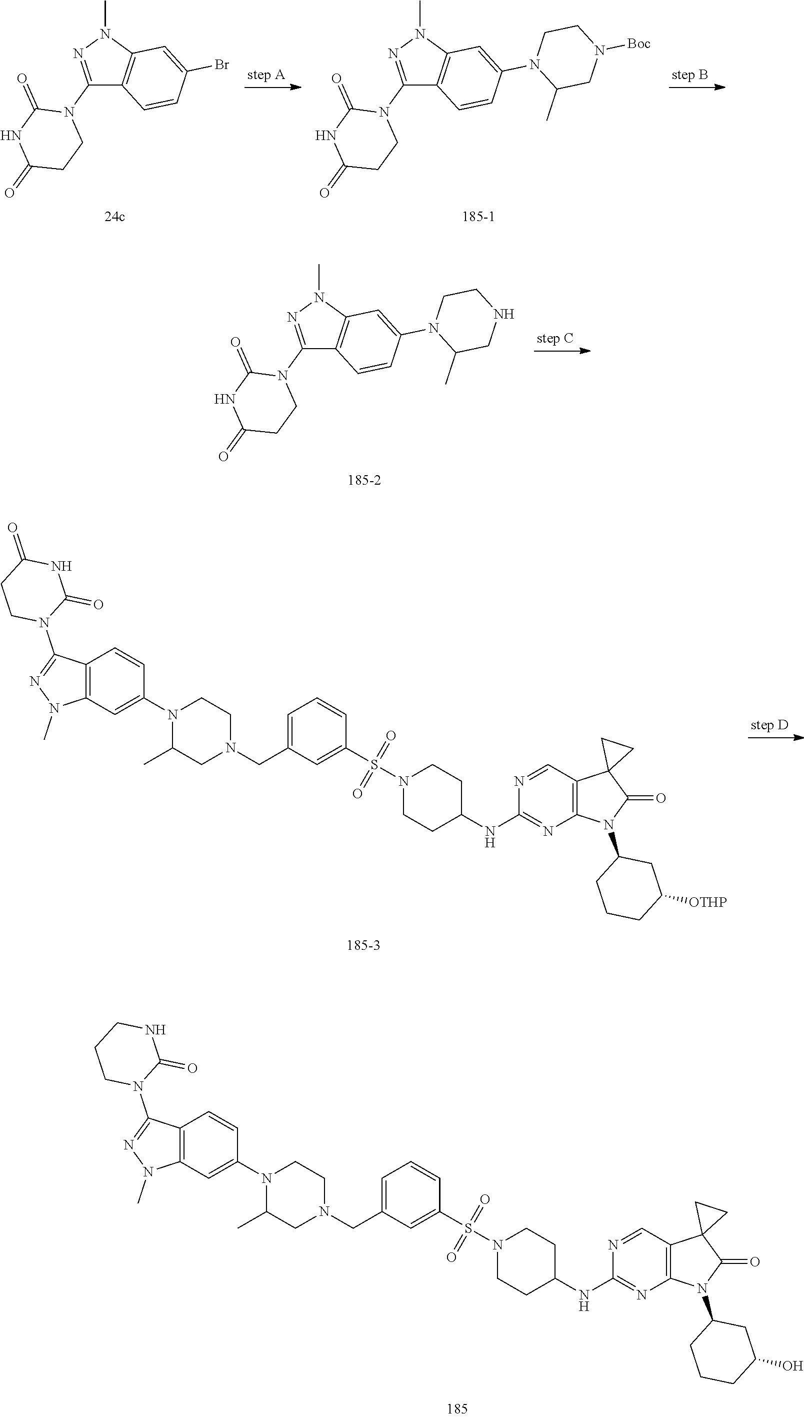

### Step A. Tert-butyl 4-[3-(2,4-dioxo-1,3-diazinan-1-yl)-1-methylindazol-6-yl]-3-methylpiperazine-1-carboxylate (185-1)

To a mixture of 24c (500 mg, 1.547 mmol, 1 equiv) and tert-butyl 3-methylpiperazine-1-carboxylate (464.84 mg, 2.321 mmol, 1.5 equiv) in dioxane (5 mL) was added Pd-PEPPSI-IPentCl 2-methylpyridine (o-picoline) (130.15 mg, 0.155 mmol, 0.10 equiv) and t-BuONa (446.10 mg, 4.641 mmol, 3 equiv) at room temperature under nitrogen atmosphere. The resulting mixture was stirred for 2 h at 100° C. under nitrogen atmosphere. Desired product could be detected by LCMS. The resulting mixture was diluted with water. The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with PE/EA (1:1) to afford 185-1 (230 mg, 30.23%) as a light yellow solid. LCMS (ESI, m/z): [M+H]⁺=443.

### Step B-D. 7′-((1R,3R)-3-hydroxycyclohexyl)-2′-((1-((3-((3-methyl-4-(1-methyl-3-(2-oxotetrahydropyrimidin-1(2H)-yl)-1H-indazol-6-yl)piperazin-1-yl)methyl)phenyl)sulfonyl)piperidin-4-yl)amino)spiro[cyclopropane-1,5′-pyrrolo[2,3-d]pyrimidin]-6′(7′H)-one (185)

Compound 185 was prepared from 185-1 and Int 7 following the procedure for the synthesis of compound 159 in example 159. LCMS (ESI, m/z): [M+H]⁺=852.5.

### Example 186: 1-[6-(1-{[1-(4-{7′-[(1R,3R)-3-Hydroxycyclohexyl]-6′-oxospiro[cyclopropane-1,5′-pyrrolo[2,3-d]pyrimidin]-2′-ylamino}piperidin-1-ylsulfonyl)piperidin-3-yl]methyl}piperidin-4-yl)-1-methylindazol-3-yl]-1,3-diazinane-2,4-dione (186)

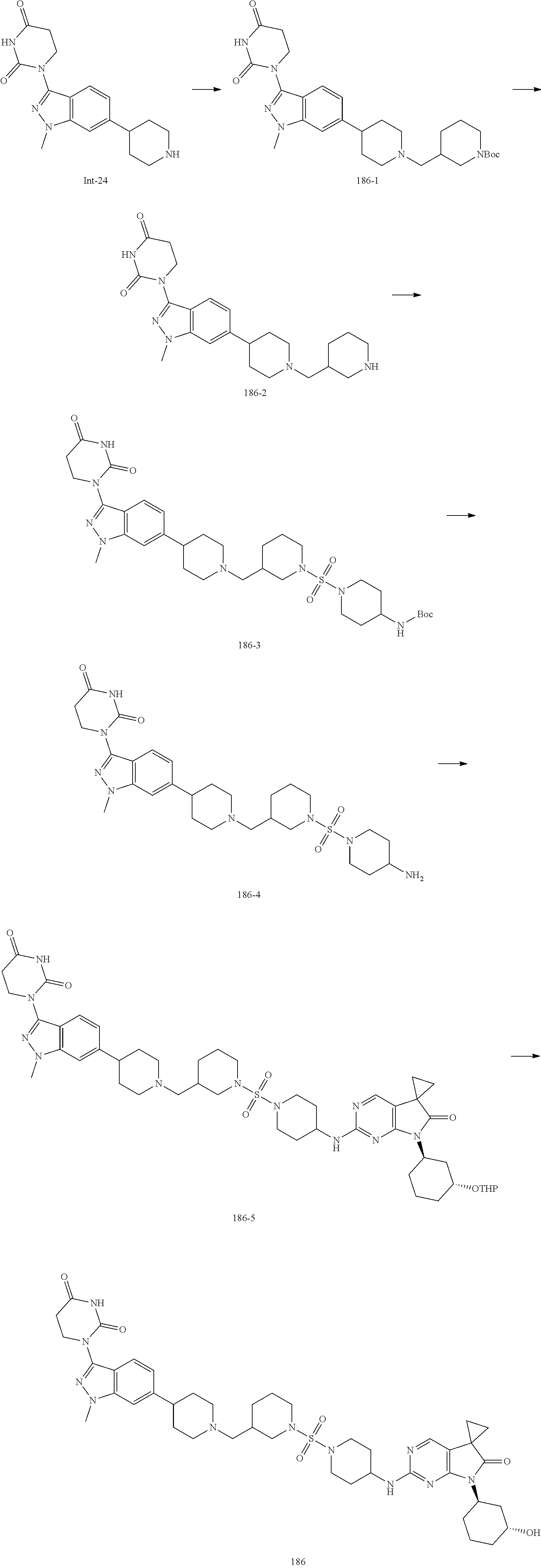

### Step A. Tert-butyl 3-((4-(3-(2,4-dioxotetrahydropyrimidin-1(2H)-yl)-1-methyl-1H-indazol-6-yl)piperidin-1-yl)methyl)piperidine-1-carboxylate (186-1)

Compound 186-1 was prepared from Int 24 and tert-butyl 3-formylpiperidine-1-carboxylate following the procedure for the synthesis of compound 119g in example 119. LCMS (ESI, m/z): [M+H]⁺=525.

### Step B. 1-(1-Methyl-6-(1-(piperidin-3-ylmethyl)piperidin-4-yl)-1H-indazol-3-yl)dihydropyrimidine-2,4(1H,3H)-dione (186-2)

A solution of 186-1 (100 mg, 0.191 mmol, 1 equiv) and 4M HCl in dioxane (1 mL) in DCM (1 mL) was stirred for 30 min at 0° C. The resulting mixture was concentrated under reduced pressure. The crude product (60 mg) was used in the next step directly without further purification. LCMS (ESI, m/z): [M+H]⁺=425.

### Step C. Tert-butyl N-{1-[3-({4-[3-(2,4-dioxo-1,3-diazinan-1-yl)-1-methylindazol-6-yl]piperidin-1-yl}methyl)piperidin-1-ylsulfonyl]piperidin-4-yl}carbamate (186-3)

To a stirred solution of 186-2 (377 mg, 0.888 mmol, 1 equiv) and 131-1 (318.38 mg, 1.066 mmol, 1.2 equiv) in DCM (5 mL) was added DIEA (573.86 mg, 4.440 mmol, 5 equiv) at 0° C. under nitrogen atmosphere. The resulting mixture was stirred for 1 h at 0° C. under nitrogen atmosphere. Desired product could be detected by LCMS. The resulting mixture was concentrated under reduced pressure. The residue was purified by reversed-phase flash chromatography with the following conditions: column, C18 silica gel; mobile phase, MeCN in Water (10 mmol/L NH₄HCO₃), 10% to 60% gradient in 30 min; detector, UV 254 nm. This resulted in 186-3 (300 mg, 49.19%) as a white solid. LCMS (ESI, m/z): [M+H]⁺=687.

### Step D. 1-[6-(1-{[1-(4-Aminopiperidin-1-ylsulfonyl)piperidin-3-yl]methyl}piperid in-4-yl)-1-methylindazol-3-yl]-1,3-diazinane-2,4-dione (186-4)

A solution of 186-3 (300 mg, 0.437 mmol, 1 equiv) and 4M HCl (gas) in 1,4-dioxane (3 mL) in ACN (3 mL) was stirred for 1 h at 0° C. under nitrogen atmosphere. Desired product could be detected by LCMS. The resulting mixture was concentrated under reduced pressure. The residue was purified by reversed-phase flash chromatography with the following conditions: column, C18 silica gel; mobile phase, MeCN in Water (10 mmol/L NH₄HCO₃), 10% to 60% gradient in 10 min; detector, UV 254 nm. This resulted in 186-4 (150 mg, 58.53%) as a white solid. LCMS (ESI, m/z): [M+H]⁺=587.

### Step E. 1-[1-Methyl-6-(1-{[1-(4-{7′-[(1R,3R)-3-(oxan-2-yloxy)cyclohexyl]-6′-oxospiro[cyclopropane-1,5′-pyrrolo[2,3-d]pyrimidin]-2′-ylamino}piperidin-1-ylsulfonyl)piperidin-3-yl]methyl}piperidin-4-yl) indazol-3-yl]-1,3-diazinane-2,4-dione (186-5)

To a solution of 186-4 (150 mg, 0.256 mmol, 1 equiv), Int-5 (96.60 mg, 0.256 mmol, 1 equiv) and RuPhos (23.86 mg, 0.051 mmol, 0.2 equiv) in dioxane (2 mL) were added Pd₂(dba)₃ (46.82 mg, 0.051 mmol, 0.2 equiv) and Cs₂CO₃ (166.59 mg, 0.512 mmol, 2 equiv) at room temperature under nitrogen atmosphere. The resulting mixture was stirred for 4 h at 100° C. under nitrogen atmosphere. The reaction was monitored by LCMS. The resulting mixture was concentrated under reduced pressure. The residue was purified by reversed-phase flash chromatography with the following conditions: column, C18 silica gel; mobile phase, MeCN in Water (10 mmol/L NH₄HCO₃), 10% to 60% gradient in 30 min; detector, UV 254 nm. This resulted in 186-5 (30 mg, 12.64%) as a yellow solid. LCMS (ESI, m/z): [M+H]⁺=928.

### Step F. 1-[6-(1-{[1-(4-{7′-[(1R,3R)-3-Hydroxycyclohexyl]-6′-oxospiro[cyclopropane-1,5′-pyrrolo[2,3-d]pyrimidin]-2′-ylamino}piperidin-1-ylsulfonyl)piperidin-3-yl]meth yl}piperidin-4-yl)-1-methylindazol-3-yl]-1,3-diazinane-2,4-dione (186)

A solution of 186-5 (30 mg, 0.032 mmol, 1 equiv) and 4M HCl (gas) in 1,4-dioxane (0.5 mL) in ACN (0.5 mL) was stirred for 1 h at 0° C. under nitrogen atmosphere. Desired product could be detected by LCMS. The resulting mixture was concentrated under reduced pressure. The residue was purified by reversed-phase flash chromatography with the following conditions: column, C18 silica gel; mobile phase, MeCN in Water (10 mmol/L NH₄HCO3), 10% to 50% gradient in 10 min; detector, UV 254 nm. This resulted in compound 186 (7 mg, 25.38%) as a white solid. LCMS (ESI, m/z): [M+H]⁺=844.4.

### Example 191&192&195: 1-(6-((R)-4-((R)-1-(3-(((3R,4S)-3-Fluoro-4-((7′-((1R,3R)-3-hydroxycyclohexyl)-6′-oxo-6′,7′-dihydrospiro[cyclopropane-1,5′-pyrrolo[2,3-d]pyrimidin]-2′-yl)amino)piperidin-1-yl)sulfonyl)phenyl)ethyl)-3-methylpiperazin-1-yl)-1-methyl-1H-indazol-3-yl)dihydropyrimidine-2,4(1H,3H)-dione (191)&1-(6-((R)-4-((S)-1-(3-(((3R,4S)-3-Fluoro-4-((7′-((1R,3R)-3-hydroxycyclohexyl)-6′-oxo-6′,7′-dihydrospiro[cyclopropane-1,5′-pyrrolo[2,3-d]pyrimidin]-2′-yl)amino)piperidin-1-yl)sulfonyl)phenyl)ethyl)-3-methylpiperazin-1-yl)-1-methyl-1H-indazol-3-yl)dihydropyrimidine-2,4(1H,3H)-dione (192)&1-(6-((3R)-4-(1-(3-(((3R,4S)-3-Fluoro-4-((7′-((1R,3R)-3-hydroxycyclohexyl)-6′-oxo-6′,7′-dihydrospiro[cyclopropane-1,5′-pyrrolo[2,3-d]pyrimidin]-2′-yl)amino)piperidin-1-yl)sulfonyl)phenyl)ethyl)-3-methylpiperazin-1-yl)-1-methyl-1H-indazol-3-yl)dihydropyrimidine-2,4(1H,3H)-dione (195)

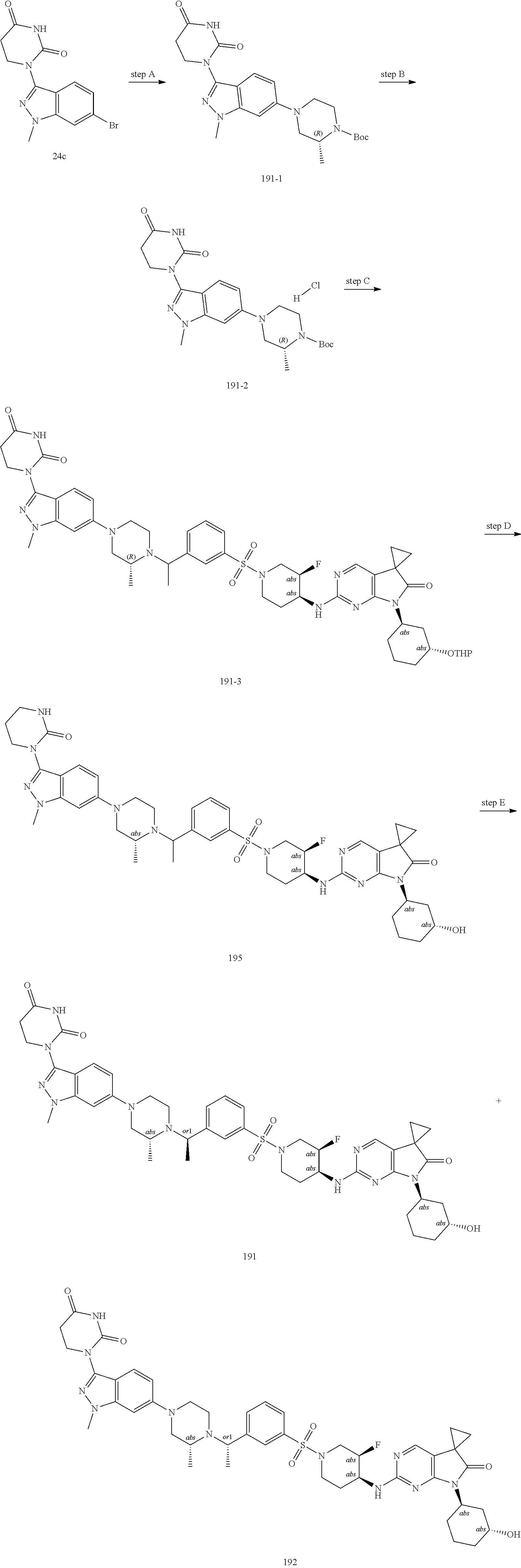

### Step A. Tert-butyl (2R)-4-[3-(2,4-dioxo-1,3-diazinan-1-yl)-1-methylindazol-6-yl]-2-methylpiperazine-1-carboxylate (191-1)

To a mixture of 24c (500 mg, 1.547 mmol, 1 equiv) and tert-butyl (2R)-2-methylpiperazine-1-carboxylate (619.78 mg, 3.094 mmol, 2 equiv) in dioxane (5 mL) were added Ruphos (72.20 mg, 0.155 mmol, 0.1 equiv), Cs₂CO₃ (1512.39 mg, 4.641 mmol, 3 equiv) and RuPhos Palladacycle Gen.3 (129.41 mg, 0.155 mmol, 0.1 equiv) at room temperature under argon atmosphere. The resulting mixture was stirred for 1 h at 100° C. under argon atmosphere. The reaction was monitored by TLC (PE/EA=2/1, Rf=0.7). The resulting mixture was concentrated under reduced pressure. The residue was purified by silica gel column chromatography, eluted with PE/EA (2:1) to afford 191-1 (300 mg, 43.81%) as a white solid. LCMS (ESI, m/z): [M+H]⁺=443.

### Step B-E. 1-(6-((R)-4-((R)-1-(3-(((3R,4S)-3-Fluoro-4-((7′-((1R,3R)-3-hydroxycyclohexyl)-6′-oxo-6′,7′-dihydrospiro[cyclopropane-1,5′-pyrrolo[2,3-d]pyrimidin]-2′-yl)amino)piperidin-1-yl)sulfonyl)phenyl)ethyl)-3-methylpiperazin-1-yl)-1-methyl-1H-indazol-3-yl)dihydropyrimidine-2,4(1H,3H)-dione (191)&1-(6-((R)-4-((S)-1-(3-(((3R,4S)-3-Fluoro-4-((7′-((1R,3R)-3-hydroxycyclohexyl)-6′-oxo-6′,7′-dihydrospiro[cyclopropane-1,5′-pyrrolo[2,3-d]pyrimidin]-2′-yl)amino)piperidin-1-yl)sulfonyl)phenyl)ethyl)-3-methylpiperazin-1-yl)-1-methyl-1H-indazol-3-yl)dihydropyrimidine-2,4(1H,3H)-dione (192)&1-(6-((3R)-4-(1-(3-(((3R,4S)-3-Fluoro-4-((7′-((1R,3R)-3-hydroxycyclohexyl)-6′-oxo-6′,7′-dihydrospiro[cyclopropane-1,5′-pyrrolo[2,3-d]pyrimidin]-2′-yl)amino)piperidin-1-yl)sulfonyl)phen yl)ethyl)-3-methylpiperazin-1-yl)-1-methyl-1H-indazol-3-yl)dihydropyrimidine-2,4(1H,3H)-dione (195)

Compound 195 was prepared from 191-1 and Int 35 following the procedure for the synthesis of compound 182 in example 182. LCMS (ESI, m/z): [M+H]⁺=884.5. Compounds 191 and 192 were obtained by Chiral-HPLC separation of compound 195.

### Example 200&201:1-(6-{1-[(1R*)-1-{2-Fluoro-5-[(3R,4S)-3-fluoro-4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl]phenyl}ethyl]piperidin-4-yl}-1-methylindazol-3-yl)-1,3-diazinane-2,4-dione (200)&1-(6-{1-[(1R*)-1-{2-Fluoro-5-[(3R,4S)-3-fluoro-4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl]phenyl}ethyl]piperidin-4-yl}-1-methylindazol-3-yl)-1,3-diazinane-2,4-dione (201)

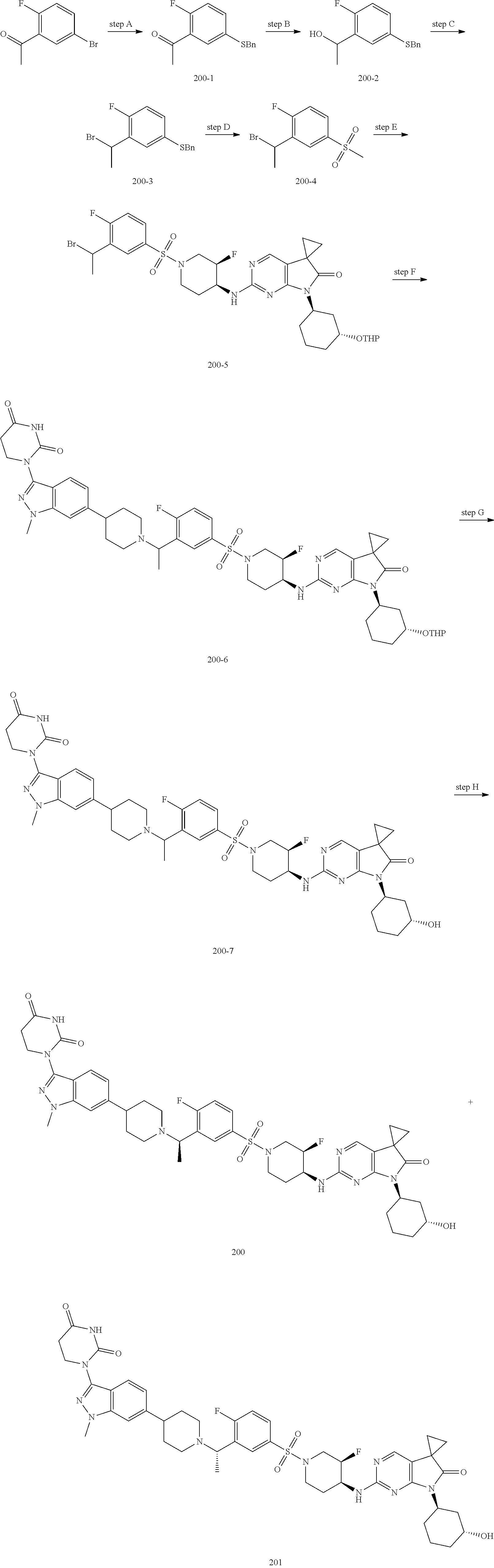

### Step A. 1-[5-(Benzylsulfanyl)-2-fluorophenyl]ethan one (200-1)

To a stirred mixture of 1-(5-bromo-2-fluorophenyl)ethanone (10 g, 46.075 mmol, 1 equiv) and benzyl mercaptan (11.45 g, 92.150 mmol, 2 equiv) in dioxane (100 mL) was added Pd₂(dba)₃ (4.22 g, 4.608 mmol, 0.1 equiv), XantPhos (4.00 g, 6.911 mmol, 0.15 equiv) and DIEA (11.91 g, 92.150 mmol, 2 equiv) at room temperature under nitrogen atmosphere. The resulting mixture was stirred for 2 h at 80° C. under nitrogen atmosphere. Desired product could be detected by LCMS. The resulting mixture was diluted with water. The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with PE/EA (5:1) to afford 200-1 (5 g, 37.52%) as a light yellow oil.

### Step B. 1-[5-(Benzylsulfanyl)-2-fluorophenyl]ethanol (200-2)

A solution of 200-1 (4.7 g, 18.054 mmol, 1 equiv) and NaBH₄ (2.05 g, 54.162 mmol, 3 equiv) in MeOH (50 mL) was stirred for 2 h at room temperature under nitrogen atmosphere. Desired product could be detected by GCMS. The reaction was quenched by the addition of sat. NH₄C1 (aq.) at 0° C. The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with PE/EA (5:1) to afford 200-2 (3.3 g, 62.71%) as a light yellow oil.

### Step C. 4-(Benzylsulfanyl)-2-(1-bromoethyl)-1-fluorobenzene (200-3)

To a stirred mixture of 200-2 (3.3 g, 12.579 mmol, 1 equiv) in AcOH (10 mL) was added HBr in AcOH (40%) (4.07 g, 50.316 mmol, 4 equiv) in portions at 0° C. under nitrogen atmosphere. The resulting mixture was stirred for overnight at room temperature under nitrogen atmosphere. Desired product could be detected by LCMS. The resulting mixture was concentrated under reduced pressure. The residue was purified by silica gel column chromatography, eluted with PE/EA (5:1) to afford 200-3 (1.2 g, 26.40%) as a yellow oil.

### Step D. 3-(1-Bromoethyl)-4-fluorobenzenesulfonyl chloride (200-4)

To a stirred solution of 200-3 (0.7 g, 2.152 mmol, 1 equiv) and AcOH (0.52 g, 8.608 mmol, 4 equiv) in DCM (10 mL) was added SO₂Cl₂ (1.16 g, 8.595 mmol, 3.99 equiv) in portions at 0° C. under nitrogen atmosphere. The resulting mixture was stirred for 1 h at 0° C. under nitrogen atmosphere. Desired product could be detected by LCMS. The resulting mixture was concentrated under reduced pressure. The residue was purified by silica gel column chromatography, eluted with PE/EA (8:1) to afford 200-4 (162 mg, 21.22%) as a light yellow oil.

### Step E. 2′-{[(3R,4S)-1-[3-(1-Bromoethyl)-4-fluorobenzenesulfonyl]-3-fluoropiperidin-4-yl]amino}-7′-[(1R,3R)-3-(oxan-2-yloxy)cyclohexyl]spiro[cyclopropane-1,5′-pyrrolo[2,3-d]pyrimidin]-6′-one (200-5)

To a stirred mixture of 35f (150 mg, 0.326 mmol, 1 equiv) and 200-4 (98.42 mg, 0.326 mmol, 1 equiv) in DCM (5 mL) was added DIEA (126.56 mg, 0.978 mmol, 3 equiv) dropwise at 0° C. under nitrogen atmosphere. The resulting mixture was stirred for 1 h at room temperature under nitrogen atmosphere. Desired product could be detected by LCMS. The resulting mixture was concentrated under reduced pressure. The residue was purified by silica gel column chromatography, eluted with PE/EA (10:1) to afford 200-5 (95 mg, 36.15%) as a light yellow oil. LCMS (ESI, m/z): [M+H]⁺=724.

### Step F-H. 1-(6-{1-[(1R*)-1-{2-Fluoro-5-[(3R,4S)-3-fluoro-4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl]phenyl}ethyl]piperidin-4-yl}-1-methylindazol-3-yl)-1,3-diazinane-2,4-dione (200)&1-(6-{1-[(1R*)-1-{2-Fluoro-5-[(3R,4S)-3-fluoro-4-{7′-[(1R,3R)-3-hydroxycyclohexyl]-6′-oxospiro[cyclopropane-1,5′-pyrrolo[2,3-d]pyrimidin]-2′-ylamino}piperidin-1-ylsulfonyl]phenyl}ethyl]piperidin-4-yl}-1-methylindazol-3-yl)-1,3-diazinane-2,4-dione (201)

Compound 200 and 201 was prepared from 200-5 and Int 24 following the procedure for the synthesis of compound 159 in example 159 and Chiral-HPLC separation. LCMS (ESI, m/z): [M+H]⁺=887.3.

Compounds 3 to 12 were prepared as described for compound 1 and 2 from common intermediates. Compounds 15 to 115, 129 were prepared as described for Example 22. Compounds 116 and 128 were prepared as described for Example 116. Compounds 118, 120 and 123 were prepared as described for Example 118. Compounds 125 and 127 were prepared as described for Example 125. Compounds 137 and 139 were prepared as described for Example 135. Compounds 142, 144 and 145 were prepared as described for Example 140. Compound 143 was prepared as described for Example 138. Compound 148 was prepared as described for Example 147. Compound 150 was prepared as described for Example 149. Compounds 154 and 157 were prepared as described for Example 153. Compounds 156 and 158 were prepared as described for Example 155. Compound 168 was prepared as described for Example 161. Compound 171 was prepared as described for Example 167. Compound 172 was prepared as described for Example 170 and 169 from 2-fluoro-3-methylaniline. Compound 178 was prepared as described for Example 164. Compound 184 was prepared as described for Example 182. Compounds 187 and 188, 189 were prepared as described for Example 182 using Int-35. Compounds 196 and 197, 198 and 199 were obtained by Chiral-HPLC separation of compound 187 and 188. Compound 190 was prepared as described for Example 183. Compounds 193 and 194 were prepared as described for Example 191. Compounds 202 and 203, 204 and 205, 206 and 207, 208 and 209 were prepared as described for Example 185 using Int-35 and Chiral-HPLC separation. Compounds 210 and 211 were prepared as described for Example 200 using Int-34 and Chiral-HPLC separation.

TABLE 2
Structure, physicochemical data for compounds 1 to 211

| | | Observed | |
|---|---|---|---|
| NO. | Structure | [M + H]⁺ | ¹H NMR Data |
| 1 | 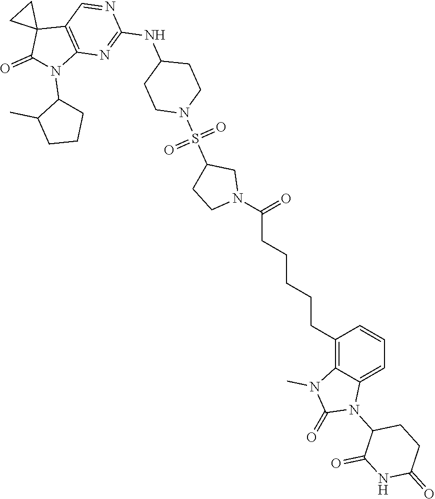 [SMILES: CC1CCCC1N1C(=O)C2(CC2)c2cnc(NC3CCN(S(=O)(=O)C4CCN(C(=O)CCCCCc5cccc6c5n(C)c(=O)n6C5CCC(=O)NC5=O)C4)CC3)nc21] | 830.3 | ¹H NMR (400 MHz, DMSO-d6) δ 11.08 (s, 1H), 7.76 (d, J = 3.6 Hz, 1H), 7.09 (s, 1H), 7.01-6.76 (m, 3H), 5.47-5.21 (m, 1H), 5.01-4.15 (m, 1H), 4.11-3.78 (m, 3H), 3.69-3.58 (m, 4H), 3.58-3.41 (m, 5H), 3.18-2.79 (m, 5H), 2.78-2.57 (m, 3H), 2.36-2.14 (m, 6H), 2.13- 2.04 (m, 1H), 2.03-1.74 (m, 6H), 1.83-1.65 (m, 1H), 1.65-1.49 (m, 7H), 1.49-1.32 (m, 5H), 1.31-1.11 (m, 2H), 0.88 (d, J = 6.8 Hz, 2H), 0.74 (d, J = 6.8 Hz, 1H). |

| | | | |
|---|---|---|---|
| 2 | 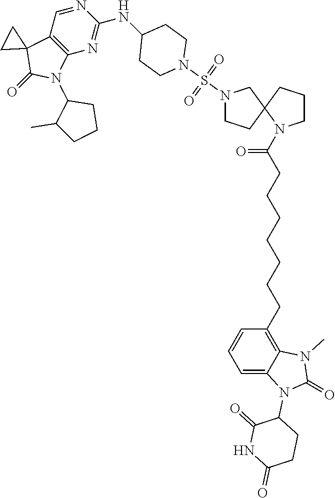 [SMILES: CC1CCCC1N1C(=O)C2(CC2)c2cnc(NC3CCN(S(=O)(=O)N4CCC5(CCCN5C(=O)CCCCCCCc5cccc6c5n(C)c(=O)n6C5CCC(=O)NC5=O)C4)CC3)nc21] | 913.5 | ¹H NMR (400 MHz, DMSO-d6) δ 11.05 (s, 1H), 7.75 (d, J = 3.4 Hz, 1H), 7.31-6.49 (m, 4H), 5.35 (m, 1H), 4.97-4.04 (m, 1H), 3.88-3.69 (m, 2H), 3.60 (d, J = 11.4 Hz, 2H), 3.54 (s, 3H), 3.51-3.40 (m, 3H), 3.24 (q, J = 8 Hz, 1H), 3.05-2.55 (m, 10H), 2.22 (t, J = 7.3 Hz, 3H), 2.09-1.73 (m, 9H), 1.72-1.11 (m, 20H), 0.88 (d, J = 6.8 Hz, 2H), 0.73 (d, J = 6.8 Hz, 1H). |
| 3 | 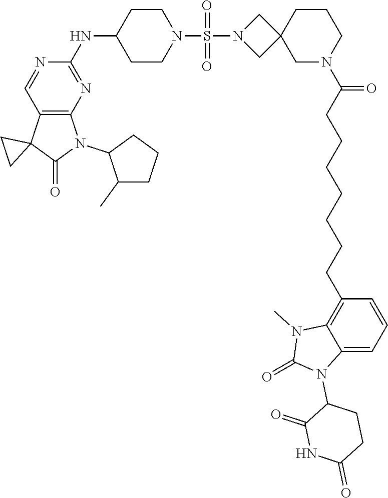 [SMILES: CC1CCCC1N1C(=O)C2(CC2)c2cnc(NC3CCN(S(=O)(=O)N4CC5(CCCN(C(=O)CCCCCCCc6cccc7c6n(C)c(=O)n7C6CCC(=O)NC6=O)C5)C4)CC3)nc21] | 913.2 | ¹H NMR (300 MHz, DMSO-d6) δ 11.07 (s, 1H), 7.76 (d, J = 2.4 Hz, 1H), 7.08 (s, 1H), 7.02-6.90 (m, 2H), 6.90-6.76 (m, 1H), 5.36 (m, 1H), 4.87-4.06 (m, 1H), 3.82 (s, 1H), 3.72-3.42 (m, 11H), 3.39-3.35 (m, 1H), 3.05-2.80 (m, 5H), 2.78-2.57 (m, 3H), 2.42-2.10 (m, 3H), 2.06-1.79 (m, 6H), 1.79-1.64 (m, 3H), 1.65-1.42 (m, 12H), 1.43-1.13 (m, 8H), 0.81 (d, 6.8 Hz, 3H). |
| 4 | 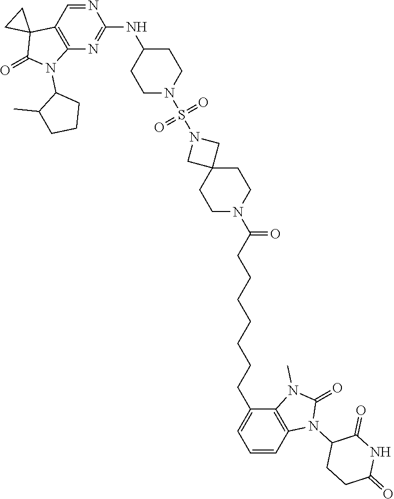 [SMILES: CC1CCCC1N1C(=O)C2(CC2)c2cnc(NC3CCN(S(=O)(=O)N4CC5(CCN(C(=O)CCCCCCCc6cccc7c6n(C)c(=O)n7C6CCC(=O)NC6=O)CC5)C4)CC3)nc21] | 913.4 | ¹H NMR (400 MHz, DMSO-d6) δ 10.98 (s, 1H), 7.77 (d, J = 3.7 Hz, 1H), 7.31-6.60 (m, 4H), 5.36 (dd, J = 12.7, 5.4 Hz, 1H), 4.99-4.01 (m, 1H), 3.83 (s, 1H), 3.57 (d, J = 12.1 Hz, 9H), 3.16-2.78 (m, 5H), 2.77-2.69 (m, 2H), 2.70-2.56 (m, 3H), 2.36-2.14 (m, 3H), 1.94 (m, 6H), 1.72-1.15 (m, 23H), 0.81 (d, 6.8 Hz, 3H). |
| 5 | 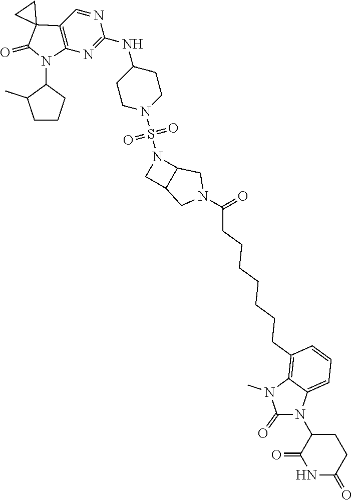 [SMILES: CC1CCCC1N1C(=O)C2(CC2)c2cnc(NC3CCN(S(=O)(=O)N4CC5CN(C(=O)CCCCCCCc6cccc7c6n(C)c(=O)n7C6CCC(=O)NC6=O)CC54)CC3)nc21] | 885.3 | ¹H NMR (300 MHz, DMSO-d6) δ 11.08 (s, 1H), 7.75 (t, J = 2.3 Hz, 1H), 7.08 (s, 1H), 7.01-6.88 (m, 2H), 6.81 (dd, J = 7.9, 5.8 Hz, 1H), 5.36 (m, 1H), 4.71 (m, 1H), 4.19 (d, J = 9 Hz, 1H), 3.99 (m, 2H), 3.90-3.65 (m, 2H), 3.62-3.34 (m, 7H), 3.30-3.02 (m, 2H), 3.01-2.57 (m, 8H), 2.32 (m, 4H), 1.96 (m, 6H), 1.69 (s, 1H), 1.64-1.20 (m, 16H), 0.88 (d, J = 6.3 Hz, 2H), 0.73 (d, J = 6 Hz, 1H) |
| 6 | 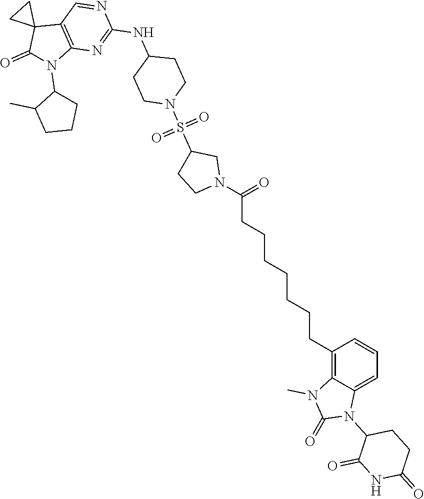 [SMILES: CC1CCCC1N1C(=O)C2(CC2)c2cnc(NC3CCN(S(=O)(=O)C4CCN(C(=O)CCCCCCCc5cccc6c5n(C)c(=O)n6C5CCC(=O)NC5=O)C4)CC3)nc21] | 858.2 | ¹H NMR (400 MHz, DMSO-d6) δ 11.05 (s, 1H), 7.76 (d, J = 3.6 Hz, 1H), 7.08 (s, 1H), 7.00-6.89 (m, 2H), 6.89- 6.81 (m, 1H), 5.36 (dd, J = 12.5, 5.4 Hz, 1H), 4.91-4.72 (m, 1H), 4.25-3.90 (m, 2H), 3.89-3.78 (m, 1H), 3.71- 3.57 (m, 4H), 3.50 (dd, J = 11.3, 5.4 Hz, 1H), 3.47-3.41 (m, 1H), 3.30-3.24 (m, 1H), 3.02 (t, J = 11.1 Hz, 2H), 2.88 (t, J = 7.9 Hz, 3H), 2.76-2.57 (m, 3H), 2.36-2.14 (m, 5H), 2.13-1.75 (m, 7H), 1.74-1.11 (m, 21H), 0.88 (d, J = 6.8 Hz, 2H), 0.74 (d, J = 6.8 Hz, 1H). |
| 7 | 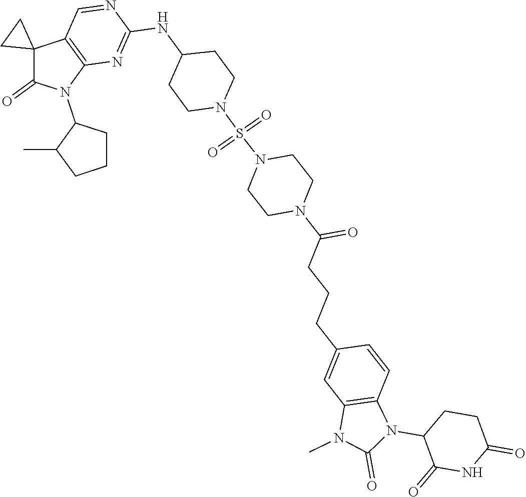 [SMILES: CC1CCCC1N1C(=O)C2(CC2)c2cnc(NC3CCN(S(=O)(=O)N4CCN(C(=O)CCCc5ccc6c(c5)n(C)c(=O)n6C5CCC(=O)NC5=O)CC4)CC3)nc21] | 817.5 | ¹H NMR (300 MHz, DMSO-d6) δ 11.09 (s, 1H), 7.76 (d, J = 2.7 Hz, 1H), 7.20-6.84 (m, 4H), 5.34 (dd, J = 12.8, 5.3 Hz, 1H), 4.83-4.13 (m, 1H), 3.83 (s, 1H), 3.52 (s, 6H), 3.28-. 81 (m, 9H), 2.80-2.56 (m, 5H), 2.36 (t, J = 7.3 Hz, 1H), 2.31-2.10 (m, 1H), 2.07-1.62 (m, 7H), 1.62- 1.38 (m, 8H), 1.25 (d, J = 6.9 Hz, 3H), 0.88 (d, J = 6.6 Hz, 2H), 0.74 (d, J = 7.0 Hz, 1H). |
| 8 | 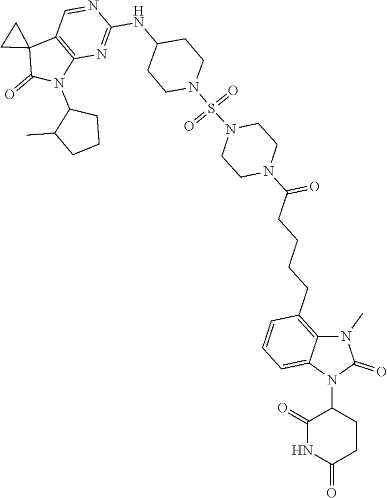 [SMILES: CC1CCCC1N1C(=O)C2(CC2)c2cnc(NC3CCN(S(=O)(=O)N4CCN(C(=O)CCCCc5cccc6c5n(C)c(=O)n6C5CCC(=O)NC5=O)CC4)CC3)nc21] | 831.2 | ¹H NMR (400 MHz, DMSO-d6) δ 11.09 (s, 1H), 7.76 (d, J = 3.6 Hz, 1H), 7.07 (s, 1H), 7.02-6.91 (m, 2H), 6.87 (m, 1H), 5.36 (m, 1H), 4.86-4.05 (m, 1H), 3.92-3.69 (m, 1H), 3.63-3.54 (m, 5H), 3.51 (s, 4H), 3.12 (d, J = 20.7 Hz, 4H), 3.02-2.82 (m, 5H), 2.78-2.66 (m, 1H), 2.67-2.56 (m, 1H), 2.39 (t, J = 5.2 Hz, 2H), 2.27-2.12 (m, 1H), 2.05-1.73 (m, 6H), 1.73-1.36 (m, 12H), 1.30- 1.14 (m, 1H), 0.88 (d, J = 6.8 Hz, 2H), 0.73 (d, J = 6.8 Hz, 1H). |
| 9 | 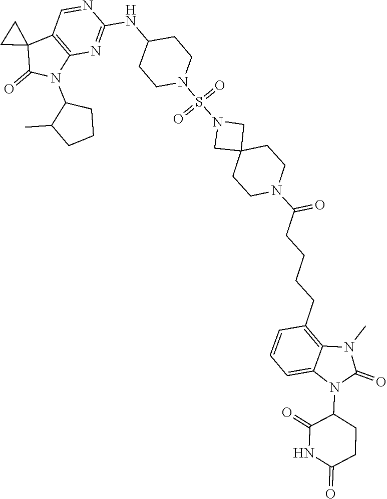 [SMILES: CC1CCCC1N1C(=O)C2(CC2)c2cnc(NC3CCN(S(=O)(=O)N4CC5(CCN(C(=O)CCCCc6cccc7c6n(C)c(=O)n7C6CCC(=O)NC6=O)CC5)C4)CC3)nc21] | 871.4 | ¹H NMR (300 MHz, DMSO-d6) δ 11.05 (s, 1H), 7.76 (d, J = 2.6 Hz, 1H), 7.08 (s, 1H), 6.99-6.90 (m, 2H), 6.86 (m, 1H), 5.36 (m, 1H), 4.86-4.04 (m, 1H), 3.82 (s, 1H), 3.69-3.48 (m, 9H), 3.44-3.34 (m, 3H), 3.02-2.80 (m, 5H), 2.76-2.56 (m, 3H), 2.35 (t, J = 6 Hz, 2H), 2.31-2.16 (m, 1H), 2.13 (s, 1H), 2.04-1.74 (m, 7H), 1.73-1.53 (m, 10H), 1.54-1.36 (m, 4H), 1.31-1.16 (m, 1H), 0.81 (d, 6.6 Hz, 3H). |
| 10 | 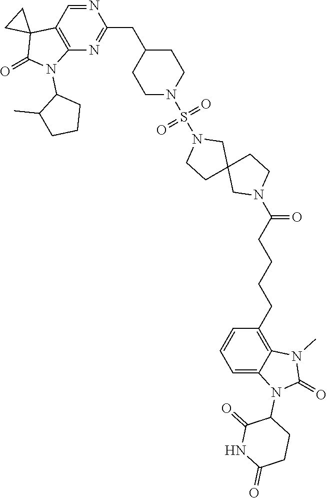 [SMILES: CC1CCCC1N1C(=O)C2(CC2)c2cnc(CC3CCN(S(=O)(=O)N4CCC5(CCN(C(=O)CCCCc6cccc7c6n(C)c(=O)n7C6CCC(=O)NC6=O)C5)C4)CC3)nc21] | 871.4 | ¹H NMR (300 MHz, DMSO-d6) δ 11.09 (s, 1H), 7.76 (t, J = 2.6 Hz, 1H), 7.07 (s, 1H), 7.00-6.91 (m, 2H), 6.91- 6.80 (m, 1H), 5.36 (m, 1H), 4.86-4.08 (m, 1H), 3.93- 3.73 (m, 1H), 3.65-3.49 (m, 6H), 3.52-3.43 (m, 2H), 3.42-3.33 (m, 3H), 3.27-3.10 (m, 3H), 2.99-2.81 (m, 5H), 2.77-2.60 (m, 2H), 2.38-2.13 (m, 4H), 2.05-1.73 (m, 10H), 1.69-1.47 (m, 8H), 1.49-1.39 (m, 2H), 1.30- 1.17 (m, 1H), 0.81 (d, 6.8 Hz, 3H). |
| 11 | 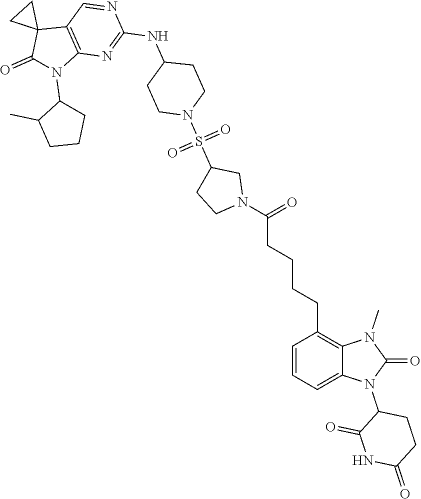 [SMILES: CC1CCCC1N1C(=O)C2(CC2)c2cnc(NC3CCN(S(=O)(=O)C4CCN(C(=O)CCCCc5cccc6c5n(C)c(=O)n6C5CCC(=O)NC5=O)C4)CC3)nc21] | 816.4 | ¹H NMR (400 MHz, DMSO-d6) δ 11.08 (s, 1H), 7.76 (d, J = 3.6 Hz, 1H), 7.09 (s, 1H), 7.01-6.91 (m, 2H), 6.86 (m, 1H), 5.36 (m, 1H), 4.84-4.13 (m, 1H), 4.02 (m, 1H), 3.84 (t, J = 9.6 Hz, 1H), 3.74-3.59 (m, 3H), 3.56 (s, 3H), 3.55-3.41 (m, 2H), 3.03 (d, J = 12.2 Hz, 2H), 2.87 (d, J = 24.0 Hz, 3H), 2.78-2.57 (m, 3H), 2.40-2.12 (m, 5H), 2.05-1.74 (m, 6H), 1.73-1.40 (m, 11H), 1.21 (d, J = 23.9 Hz, 1H), 0.88 (dd, J-6.6, 2.9 Hz, 2H), 0.74 (dd, J = 7.0, 2.1 Hz, 1H). |
| 12 | 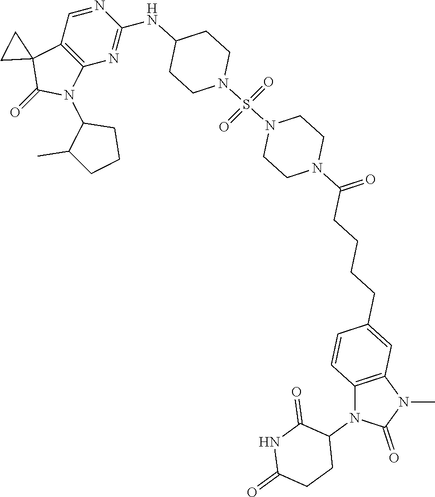 [SMILES: CC1CCCC1N1C(=O)C2(CC2)c2cnc(NC3CCN(S(=O)(=O)N4CCN(C(=O)CCCCc5ccc6c(c5)n(C)c(=O)n6C5CCC(=O)NC5=O)CC4)CC3)nc21] | 831.4 | ¹H NMR (400 MHz, DMSO-d6) δ 11.07 (s, 1H), 7.76 (d, J = 3.5 Hz, 1H), 7.12-6.97 (m, 3H), 6.87 (dd, J = 8.0, 1.6 Hz, 1H), 5.33 (dd, J = 12.8, 5.4 Hz, 1H), 4.83-4.11 (m, 1H), 3.83 (s, 1H), 3.57 (dd, J = 10.7, 5.9 Hz, 2H), 3.50 (m, 4H), 3.12 (m, 5H), 3.04-2.84 (m, 3H), 2.78- 2.56 (m, 5H), 2.52 (m, 1H), 2.35 (t, J = 7.3 Hz, 2H), 2.28-2.12 (m, 1H), 2.05-1.74 (m, 5H), 1.88-1.77(m, 2H), 1.74-1.41 (m, 12H), 1.31-1.17 (m, 1H), 0.81 (d, 6.8 Hz, 3H). |
| 13 | 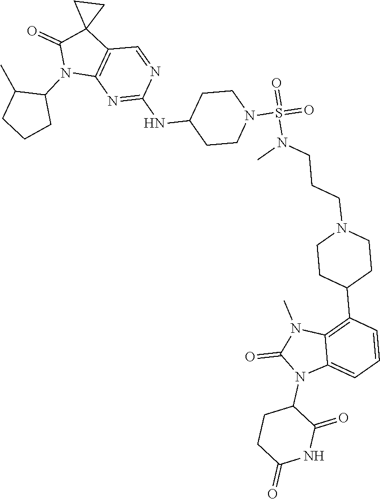 [SMILES: CC1CCCC1N1C(=O)C2(CC2)c2cnc(NC3CCN(S(=O)(=O)N(C)CCCN4CCC(c5cccc6c5n(C)c(=O)n6C5CCC(=O)NC5=O)CC4)CC3)nc21] | 817.5 | ¹H NMR (400 MHz, DMSO-d6) δ 11.07 (s, 1H), 7.76 (d, J = 3.7 Hz, 1H), 7.34-6.37 (m, 4H), 5.37 (dd, J = 12.6, 5.4 Hz, 1H), 4.90-4.07 (m, 1H), 3.80 (s, 1H), 3.72-3.45 (m, 5H), 3.27-3.11 (m, 3H), 2.99 (d, J = 10.9 Hz, 2H), 2.94-2.83 (m, 3H), 2.78 (s, 3H), 2.71 (m, 1H), 2.67-2.58 (m, 2H), 2.34 (t, J = 6.9 Hz, 2H), 2.28-2.14 (m, 1H), 2.13-2.00 (m, 3H), 1.99-1.89 (m, 4H), 1.89-1.73 (m, 4H), 1.75-1.63 (m, 4H), 1.64-1.47 (m, 4H), 1.45 (m, 1H), 1.25 (q, J = 9.9 Hz, 1H), 0.80 (d, 6.8 Hz, 3H). |
| 14 | 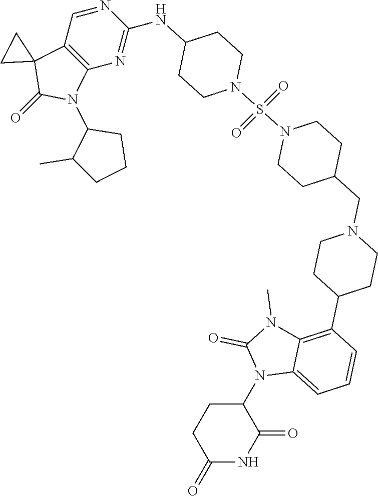 [SMILES: CC1CCCC1N1C(=O)C2(CC2)c2cnc(NC3CCN(S(=O)(=O)N4CCC(CN5CCC(c6cccc7c6n(C)c(=O)n7C6CCC(=O)NC6=O)CC5)CC4)CC3)nc21] | 843.5 | ¹H NMR (400 MHz, DMSO-d6) δ 11.02 (s, 1H), 7.76 (d, J = 3.6 Hz, 1H), 7.19-6.89 (m, 4H), 5.36 (m, 1H), 4.48 (m, 1H), 3.81 (s, 1H), 3.57 (d, J = 9.2 Hz, 7H), 3.27- 3.14 (m, 1H), 3.03-2.86 (m, 5H), 2.86-2.56 (m, 5H), 2.19 (d, J = 7.2 Hz, 3H), 2.11-1.87 (m, 7H), 1.78 (s, 6H), 1.71 (d, J = 14.1 Hz, 3H), 1.60-1.38 (m, 6H), 1.24 (s, 1H), 1.10 (q, J = 10.7 Hz, 2H), 0.88 (d, J = 6.6 Hz, 2H), 0.74 (d, J = 6.9 Hz, 1H). |
| 15 | 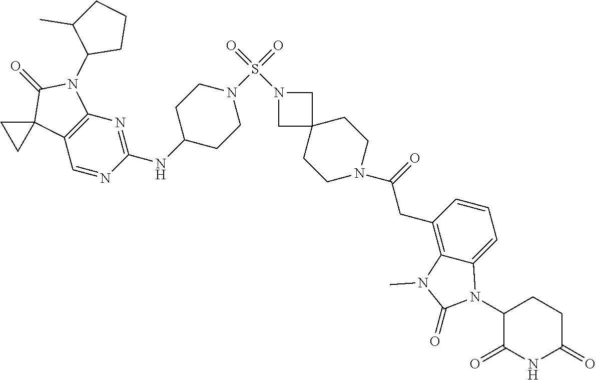 [SMILES: CC1CCCC1N1C(=O)C2(CC2)c2cnc(NC3CCN(S(=O)(=O)N4CC5(CCN(C(=O)Cc6cccc7c6n(C)c(=O)n7C6CCC(=O)NC6=O)CC5)C4)CC3)nc21] | 829.3 | ¹H NMR (400 MHz, DMSO-d₆) δ 11.04 (s, 1H), 7.70 (s, 1H), 7.12-6.81 (m, 3H), 6.71 (d, J = 7.7 Hz, 1H), 5.30 (dd, J = 12.2, 5.5 Hz, 1H), 4.79-4.07 (m, 1H), 4.01 (s, 2H), 3.87-3.65 (m, 2H), 3.64-3.42 (m, 10H), 2.94- 2.75 (m, 3H), 2.73-2.49 (m, 4H), 2.14 (s, 1H), 2.00- 1.57 (m, 11H), 1.56-1.32 (m, 7H), 1.26-1.07 (m, 1H), 0.91-0.51 (m, 3H). |
| 16 | 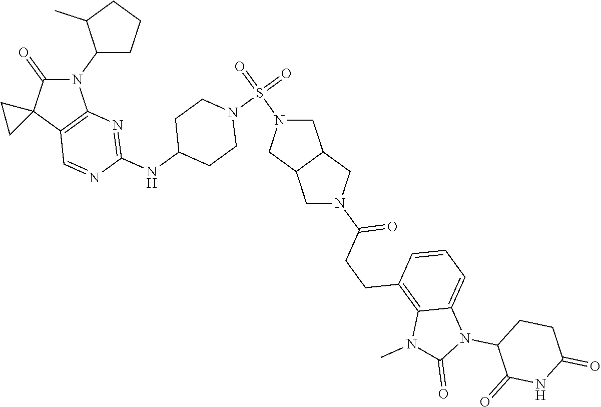 [SMILES: CC1CCCC1N1C(=O)C2(CC2)c2cnc(NC3CCN(S(=O)(=O)N4CC5CN(C(=O)CCc6cccc7c6n(C)c(=O)n7C6CCC(=O)NC6=O)CC5C4)CC3)nc21] | 829.4 | ¹H NMR (400 MHz, DMSO-d₆) 8 11.09 (s, 1H), 7.76 (d, J = 3.7 Hz, 1H), 7.23-6.63 (m, 4H), 5.36 (dd, J = 12.6, 5.4 Hz, 1H), 4.85-4.09 (m, 1H), 3.81 (s, 1H), 3.68-3.48 (m, 7H), 3.47- 3.37 (m, 2H), 3.24 (dd, J = 12.3, 5.0 Hz, 1H), 3.19-3.11 (m, 2H), 3.13-3.02 (m, 2H), 3.02- 2.79 (m, 5H), 2.79-2.53 (m, 5H), 2.29-2.11 (m, 1H), 2.05-1.62 (m, 8H), 1.62-1.39 (m, 6H), 1.31-1.13 (m, 1H), 1.07-0.41 (m, 3H). |
| 17 | 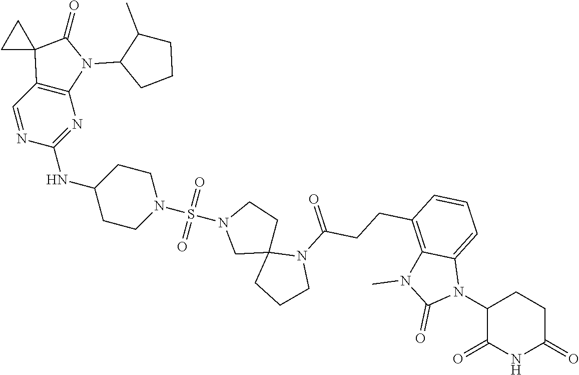 [SMILES: CC1CCCC1N1C(=O)C2(CC2)c2cnc(NC3CCN(S(=O)(=O)N4CCC5(CCCN5C(=O)CCc5cccc6c5n(C)c(=O)n6C5CCC(=O)NC5=O)C4)CC3)nc21] | 843.4 | ¹H NMR (400 MHz, DMSO-d₆) 8 11.02 (s, 1H), 7.69 (d, J = 3.9 Hz, 1H), 7.42-6.58 (m, 4H), 5.29 (dd, J = 13.1, 5.5 Hz, 1H), 4.86-4.02 (m, 1H), 3.83-3.65 (m, 2H), 3.61-3.46 (m, 4H), 3.44-3.32 (m, 3H), 3.22-3.13 (m, 1H), 3.09 (t, J = 7.6 Hz, 2H), 2.95-2.80 (m, 3H), 2.81-2.71 (m, 2H), 2.68-2.51 (m, 4H), 2.20-2.04 (m, 2H), 1.96-1.62 (m, 11H), 1.62-1.26 (m, 8H), 1.21-1.08 (m, 1H), 0.92-0.36 (m, 3H). |
| 18 | 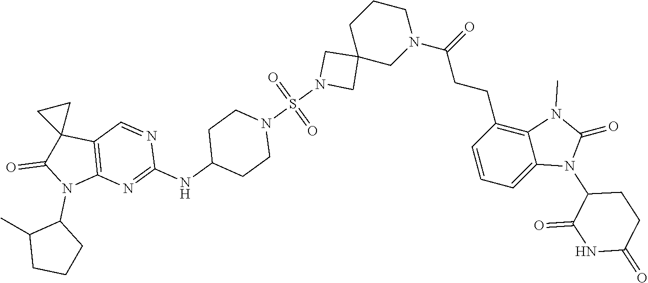 [SMILES: CC1CCCC1N1C(=O)C2(CC2)c2cnc(NC3CCN(S(=O)(=O)N4CC5(CCCN(C(=O)CCc6cccc7c6n(C)c(=O)n7C6CCC(=O)NC6=O)C5)C4)CC3)nc21] | 843.3 | ¹H NMR (400 MHz, DMSO-d₆) 8 11.08 (s, 1H), 7.77 (d, J = 3.7 Hz, 1H), 7.10 (s, 1H), 7.01-6.84 (m, 3H), 5.37 (dd, J = 12.9, 5.4 Hz, 1H), 4.90- 4.00 (m, 1H), 3.82 (s, 1H), 3.59 (d, J = 10.4 Hz, 6H), 3.50 (s, 2H), 3.43 (s, 4H), 3.30 (s, 1H), 3.17 (t, J = 7.7 Hz, 1H), 2.98-2.82 (m, 3H), 2.81-2.56 (m, 5H), 2.20 (s, 1H), 2.04-1.62 (m, 9H), 1.62- 1.37 (m, 7H), 1.37-1.29 (m, 1H), 1.23 (s, 1H), 0.87 (t, J = 6.8 Hz, 2H), 0.73 (t, J = 5.8 Hz, 1H). |
| 19 | 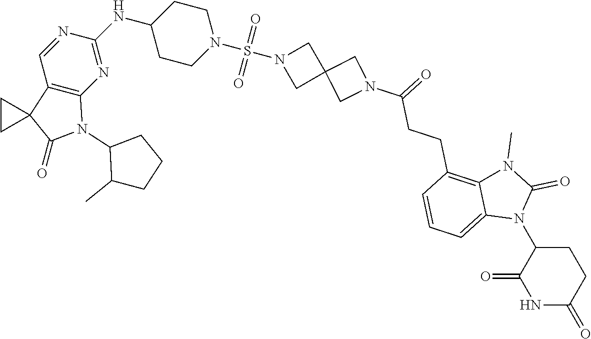 [SMILES: CC1CCCC1N1C(=O)C2(CC2)c2cnc(NC3CCN(S(=O)(=O)N4CC5(CN(C(=O)CCc6cccc7c6n(C)c(=O)n7C6CCC(=O)NC6=O)C5)C4)CC3)nc21] | 815.4 | ¹H NMR (400 MHz, DMSO-d₆) δ 7.76 (d, J = 3.5 Hz, 1H), 7.07 (s, 1H), 7.02-6.93 (m, 2H), 6.88 (dd, J = 6.3, 2.7 Hz, 1H), 5.37 (dd, J = 12.6, 5.4 Hz, 1H), 4.84-4.11 (m, 3H), 3.99 (s, 2H), 3.96-3.76 (m, 5H), 3.64-3.47 (m, 5H), 3.11 (t, J = 7.8 Hz, 2H), 2.88 (q, J = 10.0, 7.0 Hz, 3H), 2.79-2.54 (m, 3H), 2.39 (t, J = 7.8 Hz, 2H), 2.30-2.12 (m, 1H), 2.05-1.64 (m, 7H), 1.62- 1.53 (m, 2H), 1.53-1.39 (m, 4H), 1.32-1.13 (m, 1H), 0.88 (d, J = 6.6 Hz, 2H), 0.74 (d, J = 7.0 Hz, 1H). |
| 20 | 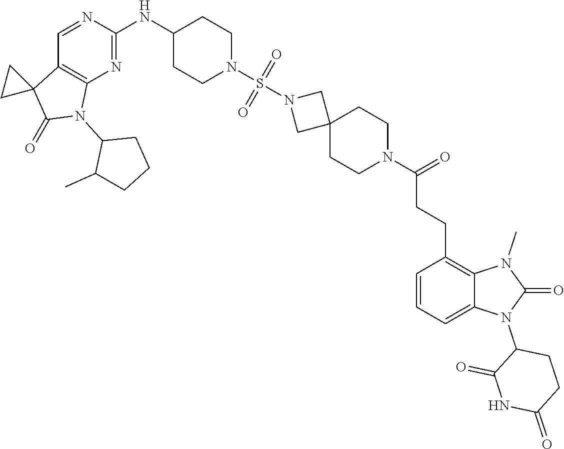 [SMILES: CC1CCCC1N1C(=O)C2(CC2)c2cnc(NC3CCN(S(=O)(=O)N4CC5(CCN(C(=O)CCc6cccc7c6n(C)c(=O)n7C6CCC(=O)NC6=O)CC5)C4)CC3)nc21] | 843.3 | ¹H NMR (300 MHz, DMSO-d₆) δ 11.08 (s, 1H), 7.76 (d, J = 2.7 Hz, 1H), 7.08 (s, 1H), 7.00- 6.86 (m, 3H), 5.37 (dd, J = 12.6, 5.4 Hz, 1H), 4.82-4.05 (m, 1H), 3.81 (s, 1H), 3.56 (d, J = 3.4 Hz, 9H), 3.44-3.38 (m, 2H), 3.37-3.34 (m, 2H), 3.14 (t, J = 7.6 Hz, 2H), 3.00-2.80 (m, 3H), 2.79-2.56 (m, 5H), 2.34-2.11 (m, 1H), 2.05-1.72 (m, 6H), 1.75-1.64 (m, 1H), 1.64- 1.35 (m, 10H), 1.33-1.07 (m, 1H), 1.00-0.47 (m, 3H). |
| 21 | 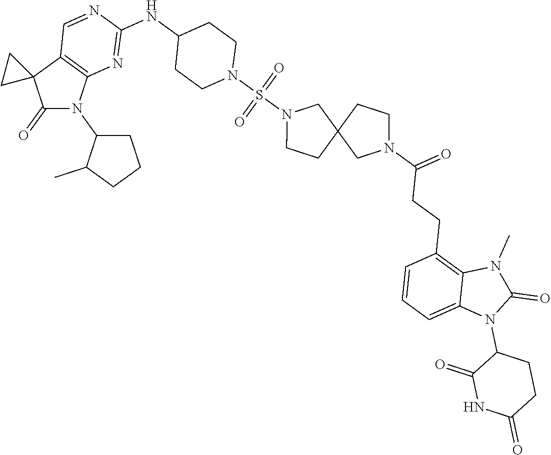 [SMILES: CC1CCCC1N1C(=O)C2(CC2)c2cnc(NC3CCN(S(=O)(=O)N4CCC5(CCN(C(=O)CCc6cccc7c6n(C)c(=O)n7C6CCC(=O)NC6=O)C5)C4)CC3)nc21] | 843.4 | ¹H NMR (400 MHz, DMSO-d₆) δ 10.98 (s, 1H), 7.99-7.62 (m, 1H), 7.33-6.28 (m, 4H), 5.36 (dd, J = 12.6, 5.3 Hz, 1H), 4.88-4.05 (m, 1H), 3.81 (s, 1H), 3.65-3.53 (m, 5H), 3.50-3.42 (m, 2H), 3.29-3.21 (m, 2H), 3.20-3.08 (m, 4H), 3.01-2.81 (m, 3H), 2.78-2.57 (m, 5H), 2.31-2.06 (m, 2H), 2.04-1.65 (m, 12H), 1.63- 1.37 (m, 6H), 1.32-1.15 (m, 1H), 0.88 (dd, J = 6.7, 2.9 Hz, 2H), 0.74 (dd, J = 7.1, 3.0 Hz, 1H). |
| 22 | 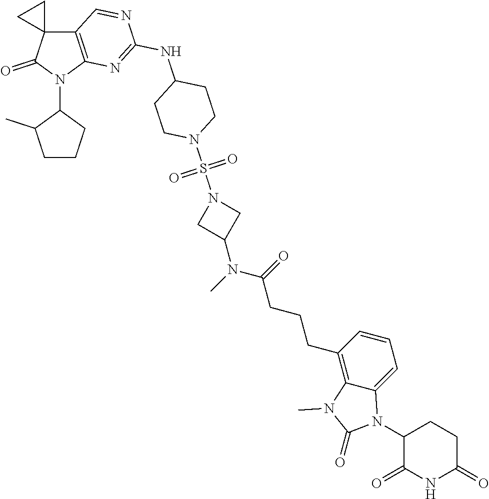 [SMILES: CC1CCCC1N1C(=O)C2(CC2)c2cnc(NC3CCN(S(=O)(=O)N4CC(N(C)C(=O)CCCc5cccc6c5n(C)c(=O)n6C5CCC(=O)NC5=O)C4)CC3)nc21] | 817.5 | ¹H NMR (400 MHz, DMSO-d₆) δ 11.08 (s, 1H), 7.76 (d, J = 3.5 Hz, 1H), 7.16-7.01 (m, 1H), 6.97 (d, J = 4.6 Hz, 2H), 6.91-6.82 (m, 1H), 5.36 (dd, J = 12.6, 5.4 Hz, 1H), 5.18-4.61 (m, 1H), 4.19 (q, J = 8.0, 6.9 Hz, 1H), 3.98 (dt, J = 23.5, 8.0 Hz, 3H), 3.86 (t, J = 7.6 Hz, 2H), 3.67 3.46 (m, 5H), 3.36-3.31 (m, 1H), 3.00 (s, 2H), 2.98-2.76 (m, 5H), 2.77-2.57 (m, 3H), 2.45 (t, J = 7.3 Hz, 2H), 2.30-2.13 (m, 1H), 2.04-1.74 (m, 8H), 1.69 (s, 1H), 1.61-1.31 (m, 6H), 1.24 (t, J = 10.1 Hz, 1H), 0.88 (d, J = 6.6 Hz, 2H), 0.74 (d, J = 7.0 Hz, 1H). |
| 23 | 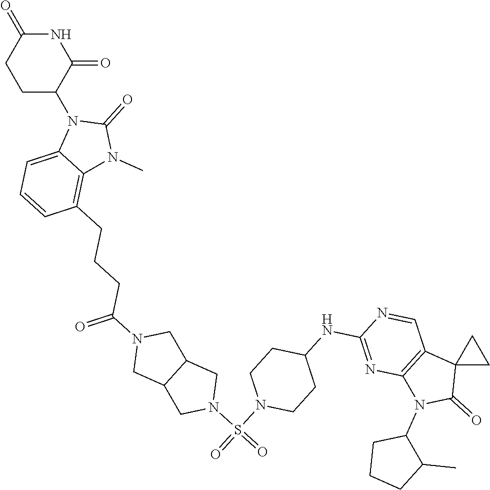 [SMILES: CC1CCCC1N1C(=O)C2(CC2)c2cnc(NC3CCN(S(=O)(=O)N4CC5CN(C(=O)CCCc6cccc7c6n(C)c(=O)n7C6CCC(=O)NC6=O)CC5C4)CC3)nc21] | 843.3 | ¹H NMR (400 MHz, DMSO-d₆) δ 11.08 (s, 1H), 7.75 (d, J = 3.4 Hz, 1H), 7.18-6.68 (m, 4H), 5.36 (dd, J = 12.6, 5.5 Hz, 1H), 4.47 (dq, J = 235.4, 8.6 Hz, 1H), 3.81 (s, 1H), 3.65 (dd, J = 10.8, 7.6 Hz, 1H), 3.62-3.51 (m, 5H), 3.44 (dt, J = 9.6, 7.6 Hz, 3H), 3.26 (dd, J = 12.3, 5.0 Hz, 2H), 3.11 (td, J = 9.6, 4.4 Hz, 2H), 3.01-2.80 (m, 7H), 2.77-2.56 (m, 3H), 2.41-2.30 (m, 2H), 2.27-2.11 (m, 1H), 2.03-1.75 (m, 8H), 1.73-1.62 (m, 1H), 1.61-1.36 (m, 6H), 1.23 (dq, J = 18.7, 10.1 Hz, 1H), 0.99-0.48 (m, 3H). |
| 24 | 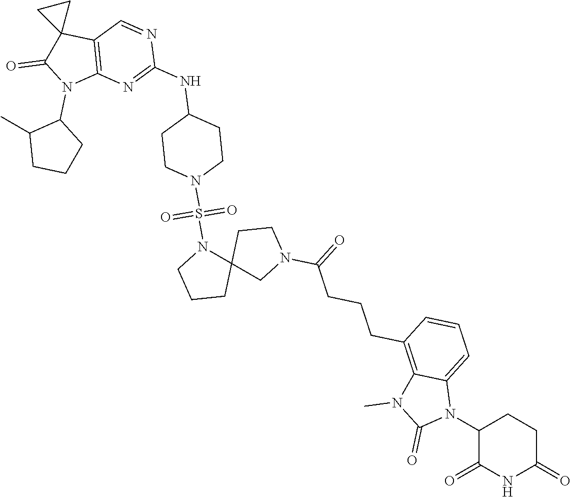 [SMILES: CC1CCCC1N1C(=O)C2(CC2)c2cnc(NC3CCN(S(=O)(=O)N4CCCC45CCN(C(=O)CCCc4cccc6c4n(C)c(=O)n6C4CCC(=O)NC4=O)C5)CC3)nc21] | 857.4 | ¹H NMR (400 MHz, DMSO-d₆) δ 11.10 (s, 1H), 7.76 (d, J = 3.7 Hz, 1H), 7.17-7.03 (m, 1H), 7.03-6.91 (m, 2H), 6.90-6.81 (m, 1H), 5.36 (dd, J = 12.6, 5.4 Hz, 1H), 4.83-4.10 (m, 1H), 3.87-3.71 (m, 1H), 3.69-3.58 (m, 1H), 3.59- 3.49 (m, 6H), 2.99-2.75 (m, 5H), 2.78-2.55 (m, 5H), 2.40-2.12 (m, 4H), 2.04-1.74 (m, 13H), 1.74-1.63 (m, 2H), 1.62-1.35 (m, 6H), 1.35-1.13 (m, 2H), 1.01-0.62 (m, 3H). |
| 25 | 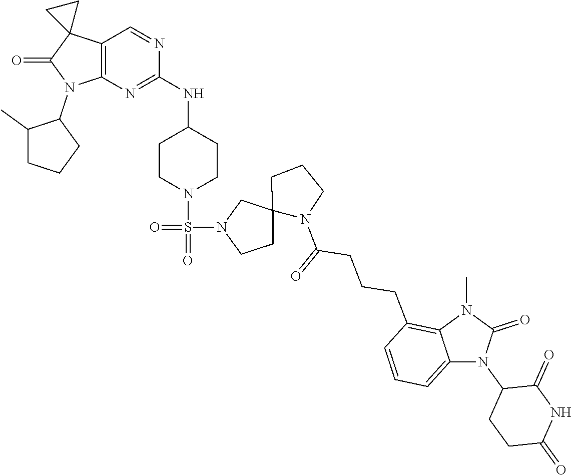 [SMILES: CC1CCCC1N1C(=O)C2(CC2)c2cnc(NC3CCN(S(=O)(=O)N4CCC5(CCCN5C(=O)CCCc5cccc6c5n(C)c(=O)n6C5CCC(=O)NC5=O)C4)CC3)nc21] | 857.4 | ¹H NMR (400 MHz, DMSO-d₆) δ 11.01 (s, 1H), 7.68 (d, J = 3.9 Hz, 1H), 7.12-6.63 (m, 4H), 5.29 (dd, J = 12.6, 5.4 Hz, 1H), 4.80-3.97 (m, 1H), 3.82-3.63 (m, 2H), 3.58-3.47 (m, 4H), 3.45-3.32 (m, 3H), 3.23-3.07 (m, 3H), 2.94- 2.70 (m, 7H), 2.69-2.49 (m, 2H), 2.34-2.24 (m, 2H), 2.19-2.03 (m, 1H), 1.98-1.65 (m, 12H), 1.66-1.52 (m, 2H), 1.53-1.29 (m, 6H), 1.23-1.03 (m, 1H), 0.93-0.54 (m, 3H). |
| 26 | 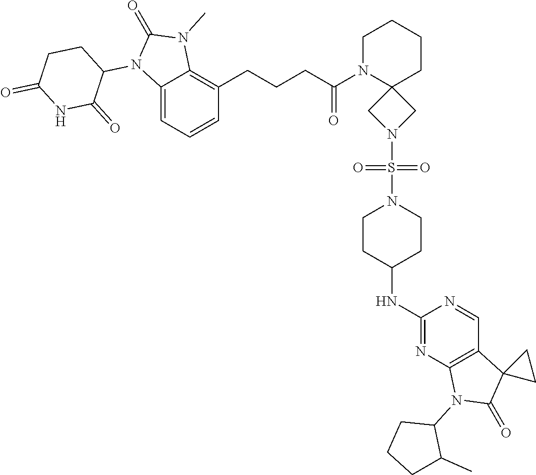 [SMILES: CC1CCCC1N1C(=O)C2(CC2)c2cnc(NC3CCN(S(=O)(=O)N4CC5(CCCCN5C(=O)CCCc5cccc6c5n(C)c(=O)n6C5CCC(=O)NC5=O)C4)CC3)nc21] | 857.4 | ¹H NMR (400 MHz, DMSO-d₆) δ 11.07 (s, 1H), 7.76 (d, J = 3.6 Hz, 1H), 7.09 (s, 1H), 6.97 (d, J = 4.7 Hz, 2H), 6.87 (q, J = 4.3 Hz, 1H), 5.36 (dd, J = 12.6, 5.4 Hz, 1H), 4.93-4.09 (m, 1H), 3.85 (d, J = 8.1 Hz, 3H), 3.69 (d, J = 8.0 Hz, 2H), 3.63-3.49 (m, 5H), 3.29-3.18 (m, 2H), 3.00- 2.81 (m, 5H), 2.78-2.56 (m, 3H), 2.43 (t, J = 7.0 Hz, 2H), 2.29-2.12 (m, 1H), 2.04-1.86 (m, 5H), 1.88-1.73 (m, 5H), 1.75-1.63 (m, 3H), 1.62-1.41 (m, 6H), 1.36 (t, J = 5.3 Hz, 2H), 1.30-1.10 (m, 1H), 1.07-0.50 (m, 3H). |
| 27 | 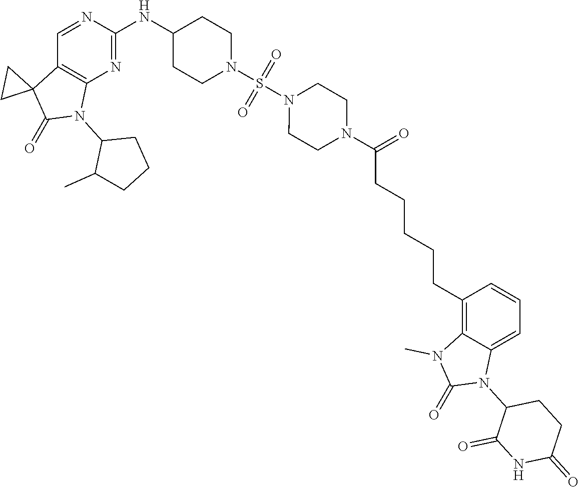 [SMILES: CC1CCCC1N1C(=O)C2(CC2)c2cnc(NC3CCN(S(=O)(=O)N4CCN(C(=O)CCCCCc5cccc6c5n(C)c(=O)n6C5CCC(=O)NC5=O)CC4)CC3)nc21] | 817.4 | ¹H NMR (400 MHz, DMSO-d₆) δ 11.08 (s, 1H), 7.76 (d, J = 3.6 Hz, 1H), 7.31-6.67 (m, 4H), 5.37 (dd, J = 12.6, 5.5 Hz, 1H), 4.88-4.08 (m, 1H), 3.83 (s, 1H), 3.67-3.41 (m, 9H), 3.14 (dt, J = 19.6, 4.4 Hz, 4H), 3.04-2.82 (m, 5H), 2.77- 2.57 (m, 3H), 2.51-2.42 (m, 2H), 2.26-2.11 (m, 1H), 2.11-1.74 (m, 8H), 173-1.62 (m, 1H), 1.62-1.35 (m, 6H), 1.31-1.10 (m, 1H), 0.99-0.49 (m, 3H). |
| 28 | 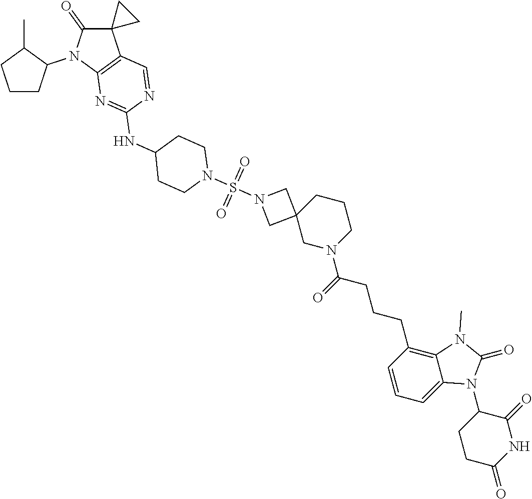 [SMILES: CC1CCCC1N1C(=O)C2(CC2)c2cnc(NC3CCN(S(=O)(=O)N4CC5(CCCN(C(=O)CCCc6cccc7c6n(C)c(=O)n7C6CCC(=O)NC6=O)C5)C4)CC3)nc21] | 857.4 | ¹H NMR (400 MHz, DMSO-d₆) δ 11.07 (s, 1H), 7.75 (d, J = 4.1 Hz, 1H), 7.27-6.93 (m, 3H), 6.90-6.81 (m, 1H), 5.36 (dd, J = 12.7, 5.3 Hz, 1H), 4.86-4.07 (m, 1H), 3.82 (s, 1H), 3.68- 3.45 (m, 11H), 3.43-3.32 (m, 2H), 3.00-2.80 (m, 5H), 2.79-2.55 (m, 3H), 2.44 (t, J = 7.1 Hz, 1H), 2.27-2.10 (m, 1H), 2.04-1.62 (m, 11H), 1.61-1.35 (m, 9H), 1.27-1.12 (m, 1H), 0.87 (d, J = 6.6 Hz, 2H), 0.73 (d, J = 6.9 Hz, 1H) |
| 29 | 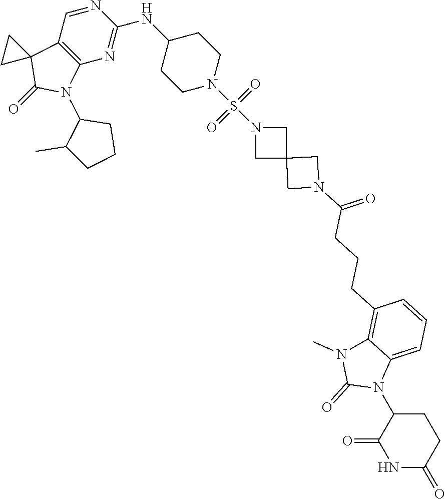 [SMILES: CC1CCCC1N1C(=O)C2(CC2)c2cnc(NC3CCN(S(=O)(=O)N4CC5(CN(C(=O)CCCc6cccc7c6n(C)c(=O)n7C6CCC(=O)NC6=O)C5)C4)CC3)nc21] | 829.4 | ¹H NMR (400 MHz, DMSO-d₆) δ 11.07 (s, 1H), 7.76 (d, J = 3.5 Hz, 1H), 7.07 (s, 1H), 6.97 (d, J = 4.6 Hz, 2H), 6.86 (q, J = 4.2 Hz, 1H), 5.36 (dd, J = 12.5, 5.4 Hz, 1H), 4.83-4.09 (m, 3H), 4.00 (s, 2H), 3.95 (s, 4H), 3.82 (s, 1H), 3.66-3.46 (m, 5H), 2.97-2.81 (m, 5H), 2.79-2.56 (m, 3H), 2.28-2.18 (m, 1H), 2.17-2.09 (m, 2H), 2.05-1.85 (m, 4H), 1.86-1.73 (m, 3H), 1.69 (s, 1H), 1.62-1.53 (m, 2H), 1.53-1.35 (m, 4H), 1.35-1.02 (m, 2H), 0.88 (d, J = 6.6 Hz, 2H), 0.74 (d, J = 7.0 Hz, 1H). |
| 30 | 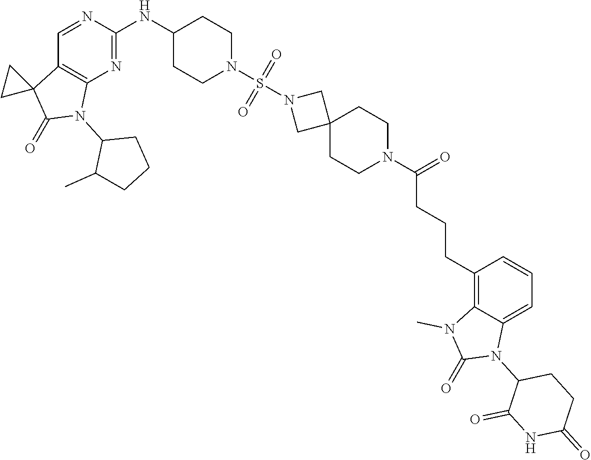 [SMILES: CC1CCCC1N1C(=O)C2(CC2)c2cnc(NC3CCN(S(=O)(=O)N4CC5(CCN(C(=O)CCCc6cccc7c6n(C)c(=O)n7C6CCC(=O)NC6=O)CC5)C4)CC3)nc21] | 857.5 | ¹H NMR (300 MHz, DMSO-d₆) δ 11.08 (s, 1H), 7.76 (d, J = 2.6 Hz, 1H), 7.21-7.01 (m, 1H), 6.97 (d, J = 4.6 Hz, 2H), 6.92-6.79 (m, 1H), 5.36 (dd, J = 12.6, 5.4 Hz, 1H), 4.88-4.06 (m, 1H), 3.82 (s, 1H), 3.58 (d, J = 5.8 Hz, 9H), 3.47- 3.32 (m, 4H), 3.05-2.80 (m, 5H), 2.79-2.56 (m, 3H), 2.43 (t, J = 6.9 Hz, 2H), 2.34-2.11 (m, 2H), 2.04-1.86 (m, 5H), 1.88-1.73 (m, 3H), 1.73-1.60 (m, 4H), 1.61-1.39 (m, 6H), 1.32- 1.14 (m, 1H), 0.97-0.49 (m, 3H). |
| 31 | 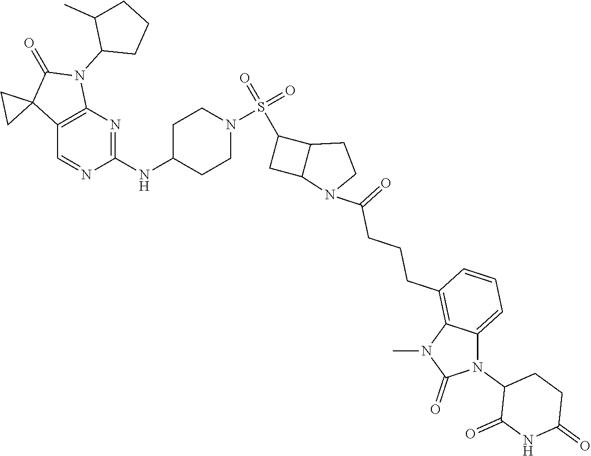 [SMILES: CC1CCCC1N1C(=O)C2(CC2)c2cnc(NC3CCN(S(=O)(=O)C4CC5C4CCN5C(=O)CCCc4cccc5c4n(C)c(=O)n5C4CCC(=O)NC4=O)CC3)nc21] | 829.4 | ¹H NMR (400 MHz, DMSO-d₆) δ 11.10 (s, 1H), 7.76 (d, J = 3.6 Hz, 1H), 7.46-6.63 (m, 4H), 5.37 (dd, J = 12.6, 5.4 Hz, 1H), 4.87 (dt, J = 42.6, 5.2 Hz, 1H), 4.79-4.13 (m, 2H), 4.13- 3.98 (m, 1H), 3.96-3.67 (m, 2H), 3.62-3.43 (m, 6H), 3.02-2.78 (m, 5H), 2.78-2.55 (m, 3H), 2.47-2.31 (m, 2H), 2.28-2.12 (m, 1H), 2.10-1.71 (m, 11H), 1.72-1.63 (m, 1H), 1.63- 1.10 (m, 7H), 0.96-0.58 (m, 3H). |
| 32 | 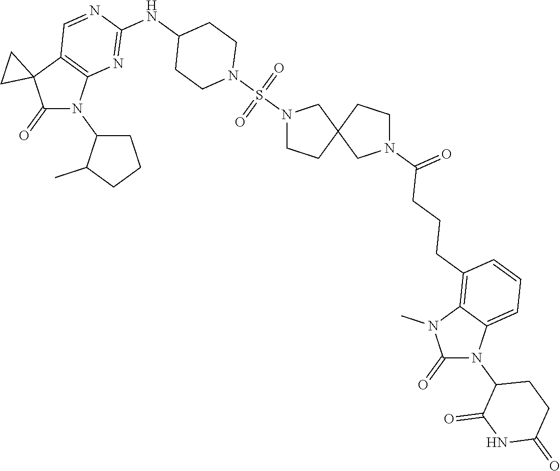 [SMILES: CC1CCCC1N1C(=O)C2(CC2)c2cnc(NC3CCN(S(=O)(=O)N4CCC5(CCN(C(=O)CCCc6cccc7c6n(C)c(=O)n7C6CCC(=O)NC6=O)C5)C4)CC3)nc21] | 857.4 | ¹H NMR (400 MHz, DMSO-d₆) δ 11.05 (s, 1H), 7.76 (d, J = 3.6 Hz, 1H), 7.07 (s, 1H), 7.03- 6.92 (m, 2H), 6.86 (dt, J = 7.2, 3.5 Hz, 1H), 5.36 (dd, J = 12.5, 5.4 Hz, 1H), 4.88-4.08 (m, 1H), 3.81 (s, 1H), 3.57 (d, J = 2.3 Hz, 5H), 3.50 (t, J = 7.0 Hz, 1H), 3.41-3.35 (m, 4H), 3.29-3.20 (m, 1H), 3.20-3.10 (m, 1H), 3.04-2.81 (m, 5H), 2.78-2.57 (m, 3H), 2.35 (q, J = 6.9 Hz, 2H), 2.28-2.15 (m, 1H), 2.03-1.75 (m, 13H), 1.72-1.32 (m, 7H), 1.31-1.17 (m, 1H), 0.96- 0.60 (m, 3H). |
| 33 | 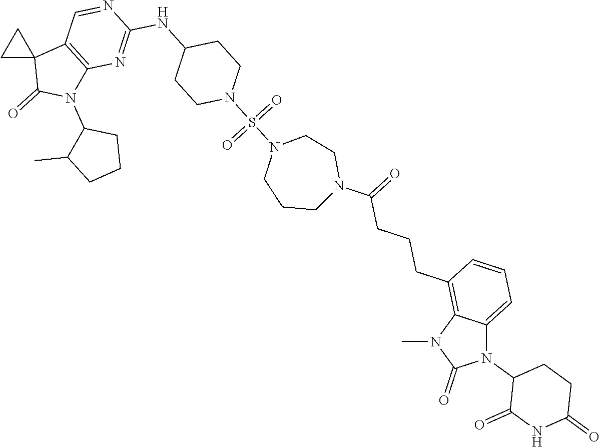 [SMILES: CC1CCCC1N1C(=O)C2(CC2)c2cnc(NC3CCN(S(=O)(=O)N4CCCN(C(=O)CCCc5cccc6c5n(C)c(=O)n6C5CCC(=O)NC5=O)CC4)CC3)nc21] | 831.4 | ¹H NMR (400 MHz, DMSO-d6) δ 11.08 (s, 1H), 7.81-7.67 (m, 1H), 7.17-6.77 (m, 4H), 5.45- 5.22 (m, 1H), 4.88-4.09 (m, 1H), 3.78 (s, 1H), 3.61-3.55 (m, 5H), 3.55-3.43 (m, 5H), 3.36- 6.77 (m, 2H), 2.96-2.78 (m, 5H), 2.76-2.57 (m, 3H), 2.47-2.38 (m, 2H), 2.27-2.13 (m, 1H), 2.04-1.88 (m, 4H), 1.89-1.68 (m, 7H), 1.63-1.39 (m, 7H), 1.29-1.17 (m, 1H), 0.87 (dd, J = 6.7, 4.0 Hz, 2H), 0.73 (dd, J = 7.0, 3.5 Hz, 1H). |
| 34 | 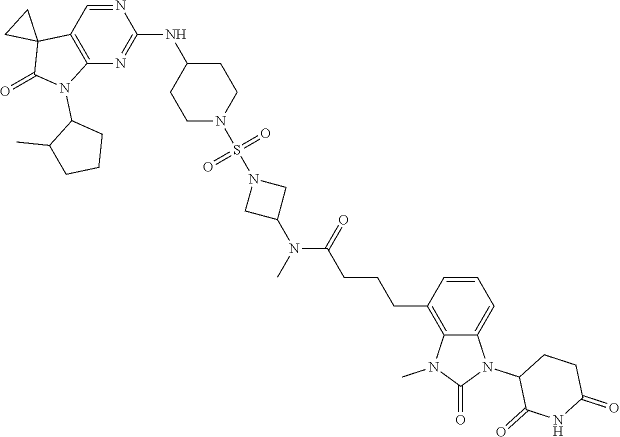 [SMILES: CC1CCCC1N1C(=O)C2(CC2)c2cnc(NC3CCN(S(=O)(=O)N4CC(N(C)C(=O)CCCc5cccc6c5n(C)c(=O)n6C5CCC(=O)NC5=O)C4)CC3)nc21] | 845.5 | ¹H NMR (400 MHz, DMSO-d6) δ 11.01 (s, 1H), 7.69 (d, J = 3.5 Hz, 1H), 7.11-6.73 (m, 4H), 5.38-5.23 (m, 1H), 5.01-4.69 (m, 1H), 4.11- 4.05 (m, 1H), 4.01-3.73 (m, 6H), 3.57-3.44 (s, 5H), 2.98-2.88 (m, 2H), 2.88-2.73 (m, 6H), 2.69-2.52 (m, 3H), 2.32-2.21 (m, 2H), 2.14-2.05 (m, 1H), 1.97-1.68 (m, 5H), 1.63- 1.25 (m, 4H), 1.23-1.06 (m, 1H), 0.81 (d, J = 6.6 Hz, 2H), 0.67 (d, J = 7.0 Hz, 1H). |
| 35 | 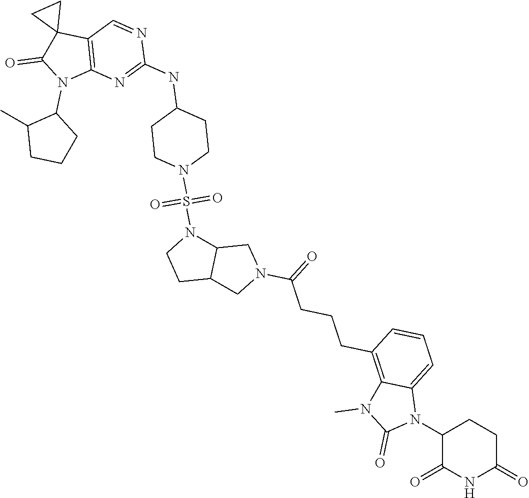 [SMILES: CC1CCCC1N1C(=O)C2(CC2)c2cnc(NC3CCN(S(=O)(=O)N4CCC5CN(C(=O)CCCc6cccc7c6n(C)c(=O)n7C6CCC(=O)NC6=O)CC54)CC3)nc21] | 885.4 | ¹H NMR (400 MHz, DMSO-d₆) δ 11.10 (s, 1H), 7.76 (d, J = 3.7 Hz, 1H), 7.10 (s, 1H), 7.04- 6.61 (m, 3H), 5.46-5.17 (m, 1H), 4.93-4.00 (m, 1H), 3.87-3.67 (m, 2H), 3.67-3.47 (m, 7H), 3.21-3.04 (m, 1H), 2.98-2.80 (m, 5H), 2.77-2.57 (m, 4H), 2.35-2.14 (m, 3H), 2.06- 1.71 (m, 12H), 1.67-1.32 (m, 13H), 1.33-1.17 (m, 1H), 1.04-0.60 (m, 4H). |
| 36 | 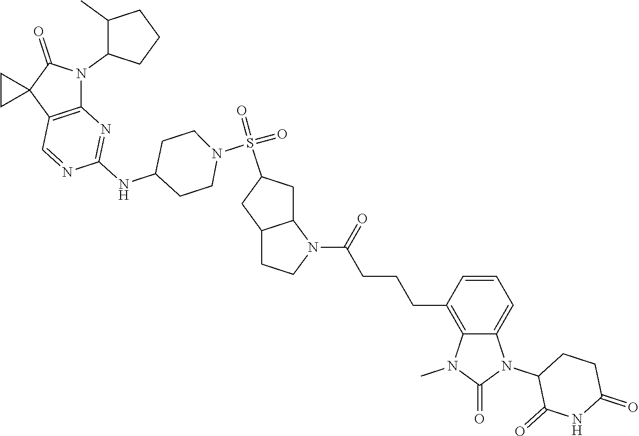 [SMILES: CC1CCCC1N1C(=O)C2(CC2)c2cnc(NC3CCN(S(=O)(=O)C4CC5CCN(C(=O)CCCc6cccc7c6n(C)c(=O)n7C6CCC(=O)NC6=O)C5C4)CC3)nc21] | 885.5 | ¹H NMR (400 MHz, DMSO-d6) δ 11.02 (s, 1H), 7.68 (d, J = 3.6 Hz, 1H), 7.10-6.65 (m, 4H), 5.29 (dd, J = 9.0, 5.1 Hz, 1H), 4.77-4.63 (m, 1H), 4.18-4.04 (m, 1H), 3.80-3.66 (m,2H), 3.66-3.51 (m, 2H), 3.50-3.44 (m, 3H), 3.41- 3.31 (m, 4H), 3.20-3.11 (m, 2H), 2.92-2.78 (m, 5H), 2.74-2.66 (m, 1H), 2.64-2.59 (m, 1H), 2.56-2.49 (m, 1H), 2.26-2.13 (m, 2H), 2.13-2.04 (m, 1H), 2.00-1.62 (m, 11H), 1.62- 1.38 (m, 9H), 1.37-1.23 (m, 4H), 1.22-1.09 (m, 1H), 0.88-059 (m, 3H). |
| 37 | 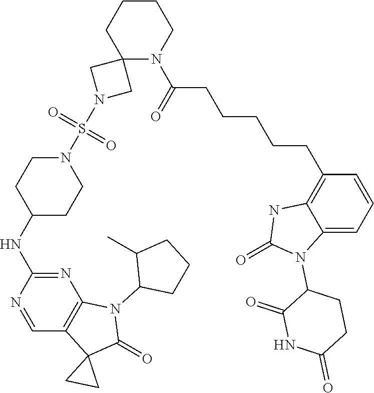 [SMILES: CC1CCCC1N1C(=O)C2(CC2)c2cnc(NC3CCN(S(=O)(=O)N4CC5(CCCCN5C(=O)CCCCCc5cccc6c5[nH]c(=O)n6C5CCC(=O)NC5=O)C4)CC3)nc21] | 885.3 | ¹H NMR (400 MHz, DMSO-d6) δ 11.06 (s, 1H), 7.76 (d, J = 3.5 Hz, 1H), 7.10 (s, 1H), 6.95 (d, J = 4.9 Hz, 2H), 6.86 (dd, J = 5.3, 3.7 Hz, 1H), 5.36 (dd, J = 12.5, 5.4 Hz, 1H), 4.84-4.07 (m, 1H), 3.82 (d, J = 8.1 Hz, 3H), 3.67 (d, J = 8.0 Hz, 2H), 3.61-3.51 (m, 4H), 3.28-3.14 (m, 2H), 3.01-2.81 (m, 5H), 2.78-2.56 (m, 2H), 2.31 (t, J = 7.3 Hz, 2H), 2.04-1.87 (m, 5H), 1.86-1.73 (m, 4H), 1.74-1.66 (m, 3H), 1.64- 1.31 (m, 16H), 1.31-1.14 (m, 1H), 0.99-0.49 (m, 3H). |
| 38 | 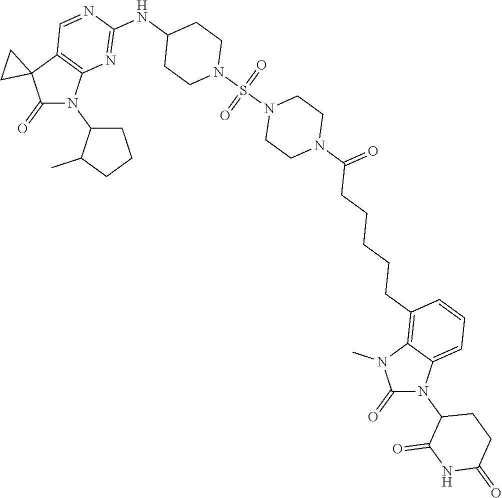 [SMILES: CC1CCCC1N1C(=O)C2(CC2)c2cnc(NC3CCN(S(=O)(=O)N4CCN(C(=O)CCCCCc5cccc6c5n(C)c(=O)n6C5CCC(=O)NC5=O)CC4)CC3)nc21] | 845.4 | ¹H NMR (400 MHz, DMSO-d6) δ 11.09 (s, 1H), 7.76 (d, J = 3.6 Hz, 1H), 7.09 (s, 1H), 7.01- 6.92 (m, 2H), 6.91-6.81 (m, 1H), 5.36 (dd, J = 12.5, 5.4 Hz, 1H), 4.97-4.07 (m, 1H), 3.83 (s, 1H), 3.62-3.52 (m, 6H), 3.54-3.44 (m, 4H), 3.12 (dt, J = 21.5, 5.4 Hz, 4H), 2.98 (t, J = 11.9 Hz, 2H), 2.94-2.82 (m, 2H), 2.71 (td, J = 12.9, 4.2 Hz, 1H), 2.66-2.56 (m, 1H), 2.34 (t, J = 7.3 Hz, 2H), 2.27-2.08 (m, 1H), 2.06-1.73 (m, 6H), 1.73-1.31 (m, 14H), 1.31-1.12 (m, 1H), 0.88 (d, J = 6.6 Hz, 2H), 0.73 (d, J = 7.0 Hz, 1H). |
| 39 | 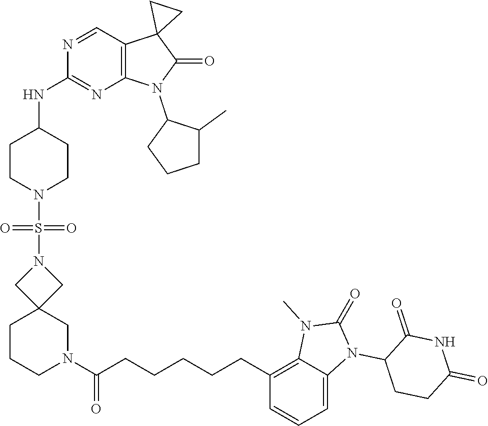 [SMILES: CC1CCCC1N1C(=O)C2(CC2)c2cnc(NC3CCN(S(=O)(=O)N4CC5(CCCN(C(=O)CCCCCc6cccc7c6n(C)c(=O)n7C6CCC(=O)NC6=O)C5)C4)CC3)nc21] | 885.5 | ¹H NMR (400 MHz, DMSO-d6) δ 11.06 (s, 1H), 7.75 (d, J = 3.5 Hz, 1H), 7.23-6.74 (m, 4H), 5.35 (dd, J = 12.8, 5.4 Hz, 1H), 4.84-4.11 (m, 1H), 3.81 (s, 1H), 3.65-3.41 (m, 11H), 3.39- 3.33 (m, 1H), 3.00-2.81 (m, 4H), 2.78-2.56 (m, 3H), 2.43-2.27 (m, 2H), 2.26-2.11 (m, 1H), 2.04-1.83 (m, 6H), 1.85-1.66 (m, 3H), 1.64-1.35 (m, 15H), 1.36-1.13 (m, 2H), 0.87 (d, J = 6.6 Hz, 2H), 0.73 (d, J = 6.9 Hz, 1H). |
| 40 | 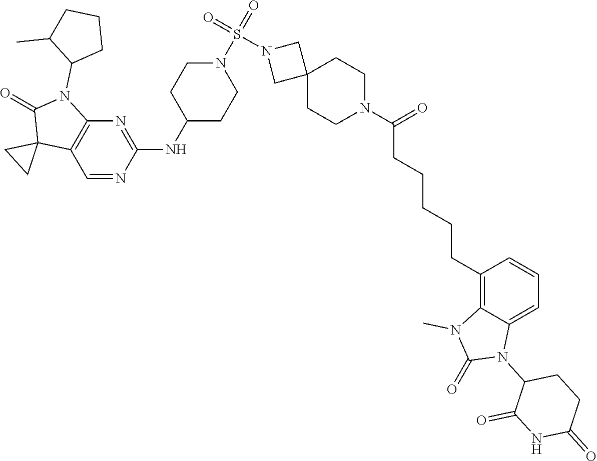 [SMILES: CC1CCCC1N1C(=O)C2(CC2)c2cnc(NC3CCN(S(=O)(=O)N4CC5(CCN(C(=O)CCCCCc6cccc7c6n(C)c(=O)n7C6CCC(=O)NC6=O)CC5)C4)CC3)nc21] | 885.4 | ¹H NMR (400 MHz, DMSO-d₆) δ 11.08 (s, 1H), 7.76 (d, J = 3.5 Hz, 1H), 7.08 (s, 1H), 7.02-6.91 (m, 2H), 6.86 (dd, J = 5.6, 3.4 Hz, 1H), 5.36 (dd, J = 12.6, 5.4 Hz, 1H), 4.81-4.13 (m, 1H), 3.82 (s, 1H), 3.56 (d, J = 11.6 Hz, 9H), 3.43-3.34 (m, 3H), 2.98-2.82 (m, 5H), 2.78-2.58 (m, 3H), 2.30 (t, J = 7.4 Hz, 2H), 2.26-2.12 (m, 1H), 2.04-1.74 (m, 6H), 1.74-1.33 (m, 18H), 1.24 (s, 1H), 0.88 (d, J = 6.6 Hz, 2H), 0.74 (d, J = 7.0 Hz, 1H). |
| 41 | 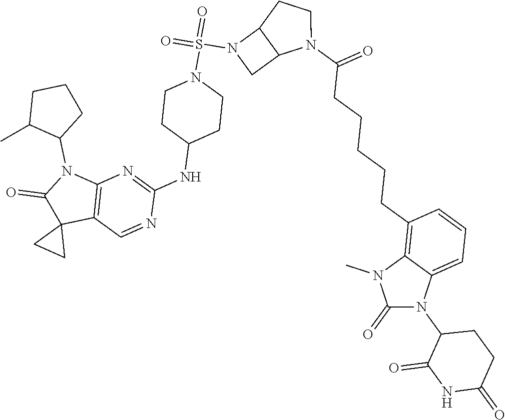 [SMILES: CC1CCCC1N1C(=O)C2(CC2)c2cnc(NC3CCN(S(=O)(=O)N4CC5C4CCN5C(=O)CCCCCc4cccc5c4n(C)c(=O)n5C4CCC(=O)NC4=O)CC3)nc21] | 857.4 | ¹H NMR (400 MHz, DMSO-d₆) δ 11.09 (s, 1H), 7.76 (dd, J = 3.7, 1.7 Hz, 1H), 7.19-6.70 (m, 4H), 5.36 (dd, J = 12.6, 5.4 Hz, 1H), 5.03-4.15 (m, 3H), 4.14-3.65 (m, 4H), 3.64-3.40 (m, 6H), 3.29 (d, J = 8.5 Hz, 1H), 3.02-2.76 (m, 5H), 2.76-2.56 (m, 3H), 2.45-2.17 (m, 3H), 2.15-2.03 (m, 1H), 2.07-1.78 (m, 7H), 1.68- 1.48 (m, 9H), 1.47-1.15 (m, 4H), 0.97-0.48 (m, 3H). |
| 42 | 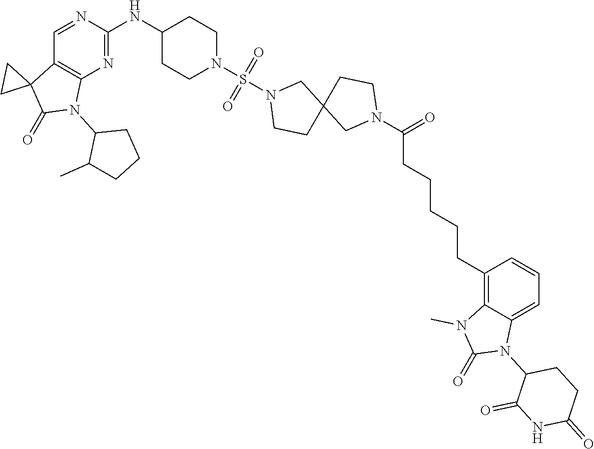 [SMILES: CC1CCCC1N1C(=O)C2(CC2)c2cnc(NC3CCN(S(=O)(=O)N4CCC5(CCN(C(=O)CCCCCc6cccc7c6n(C)c(=O)n7C6CCC(=O)NC6=O)C5)C4)CC3)nc21] | 885.4 | ¹H NMR (400 MHz, DMSO-d₆) δ 11.08 (s, 1H), 7.83-7.63 (m, 1H), 7.07 (s, 1H), 7.00-6.91 (m, 2H), 6.90-6.71 (m, 1H), 5.36 (dd, J = 12.5, 5.4 Hz, 1H), 4.93-4.06 (m, 1H), 3.81 (s, 1H), 3.68-3.46 (m, 6H), 3.44-3.34 (m, 3H), 3.26- 3.11 (m, 3H), 2.99-2.81 (m, 5H), 2.77-2.58 (m, 3H), 2.31-2.11 (m, 3H), 2.06-1.74 (m, 10H), 1.73-1.37 (m, 14H), 1.32-1.10 (m, 1H), 0.88 (d, J = 6.6 Hz, 2H), 0.74 (d, J = 7.0 Hz, 1H). |
| 43 | 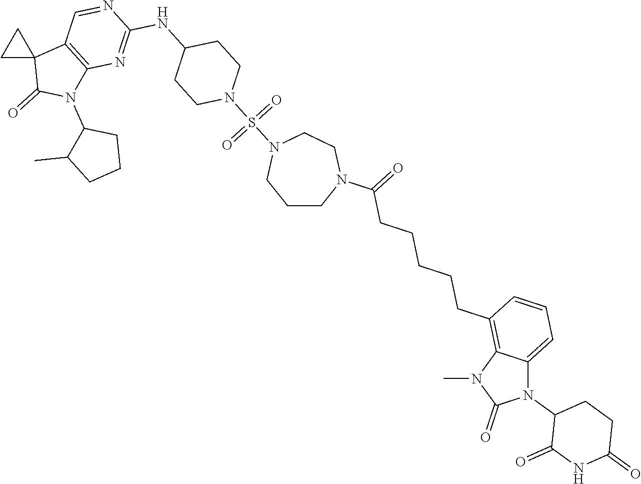 [SMILES: CC1CCCC1N1C(=O)C2(CC2)c2cnc(NC3CCN(S(=O)(=O)N4CCCN(C(=O)CCCCCc5cccc6c5n(C)c(=O)n6C5CCC(=O)NC5=O)CC4)CC3)nc21] | 859.4 | ¹H NMR (400 MHz, DMSO-d₆) δ 11.03 (s, 1H), 7.68 (d, J = 3.6 Hz, 1H), 7.12-6.62 (m, 4H), 5.27 (dd, J = 12.6, 5.3 Hz, 1H), 4.79-4.01 (m, 1H), 3.81-3.61 (m, 2H), 3.40-3.11 (m, 13H), 2.88-2.68 (m, 5H), 2.70-2.51 (m, 3H), 2.35- 2.24 (m, 2H), 2.22-2.05 (m, 1H), 1.99-1.32 (m, 20H), 1.25-1.08 (m, 1H), 0.92-0.42 (m, 3H). |
| 44 | 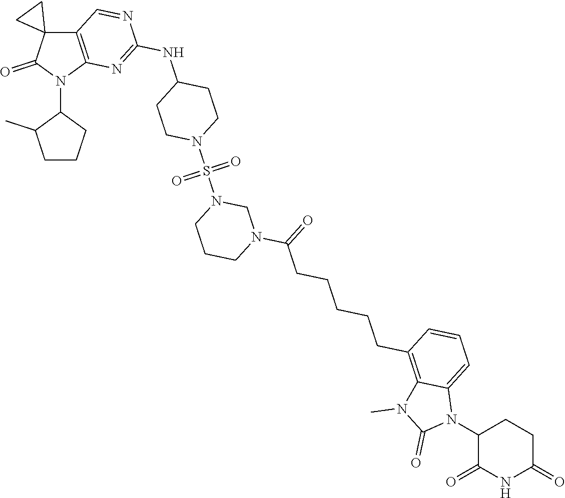 [SMILES: CC1CCCC1N1C(=O)C2(CC2)c2cnc(NC3CCN(S(=O)(=O)N4CCCN(C(=O)CCCCCc5cccc6c5n(C)c(=O)n6C5CCC(=O)NC5=O)C4)CC3)nc21] | 844.4 | ¹H NMR (400 MHz, DMSO-d₆) δ 11.08 (s, 1H), 7.76 (d, J = 3.6 Hz, 1H), 7.09 (s, 1H), 7.01- 6.63 (m, 3H), 5.44-5.16 (m, 1H), 4.86-4.53 (m, 1H), 4.31-3.77 (m, 3H), 3.66 (d, J = 12.2 Hz, 2H), 3.55 (d, J = 3.5 Hz, 3H), 3.42- 3.34 (m, 1H), 3.25-3.08 (m, 1H), 3.08-2.93 (m, 2H), 2.94-2.79 (m, 3H), 2.77-2.56 (m, 4H), 2.41-2.27 (m, 2H), 2.27-2.14 (m, 1H), 2.13- 2.03 (m, 1H), 2.03-1.31 (m, 22H), 1.31-1.12 (m, 1H), 0.88 (d, J = 6.5 Hz, 2H), 0.74 (d, J = 7.1 Hz, 1H). |
| 45 | 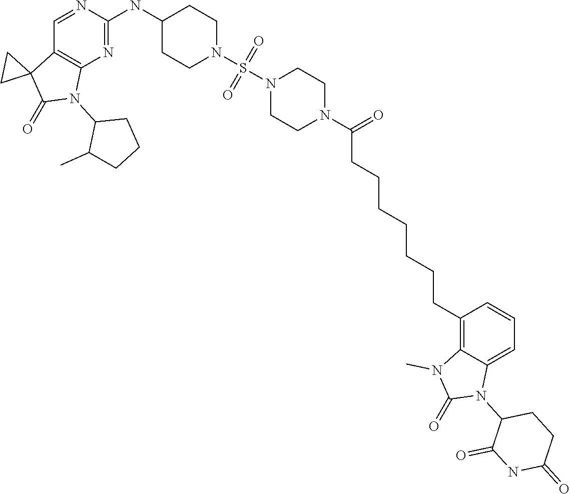 [SMILES: CC1CCCC1N1C(=O)C2(CC2)c2cnc(NC3CCN(S(=O)(=O)N4CCN(C(=O)CCCCCCCc5cccc6c5n(C)c(=O)n6C5CCC(=O)NC5=O)CC4)CC3)nc21] | 873.2 | ¹H NMR (400 MHz, DMSO-d₆) δ 11.09 (s, 1H), 7.75 (d, J = 3.6 Hz, 1H), 7.18-6.75 (m, 4H), 5.46-5.26 (m, 1H), 5.15-4.66 (m, 1H), 4.26- 3.79 (m, 6H), 3.63-3.52 (m, 5H), 3.01-2.81 (m, 9H), 2.77-2.58 (m, 3H), 2.39-1.65 (m, 12H), 1.62-1.15 (m, 18H), 0.92-0.74 (m, 3H). |
| 46 | 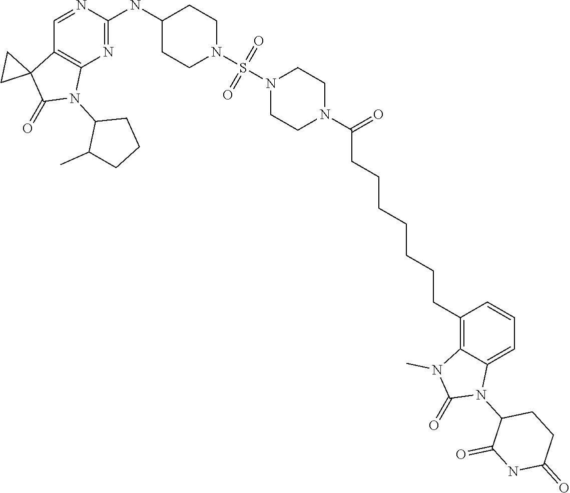 [SMILES: CC1CCCC1N1C(=O)C2(CC2)c2cnc(NC3CCN(S(=O)(=O)N4CCN(C(=O)CCCCCCCc5cccc6c5n(C)c(=O)n6C5CCC(=O)NC5=O)CC4)CC3)nc21] | 873.5 | ¹H NMR (400 MHz, DMSO-d₆) δ 11.08 (s, 1H), 7.76 (d, J = 3.6 Hz, 1H), 7.14-6.92 (m, 3H), 6.90-6.80 (m, 1H), 5.36 (dd, J = 12.6, 5.4 Hz, 1H), 4.93-4.00 (m, 1H), 3.83 (s, 1H), 3.64- 3.41 (m, 9H), 3.13 (d, J = 21.1 Hz, 4H), 3.05- 2.81 (m, 5H), 2.78-2.58 (m, 3H), 2.31 (t, J = 7.4 Hz, 2H), 2.28-2.13 (m, 1H), 2.03-1.76 (m, 6H), 1.74-1.41 (m, 12H), 1.41-1.22 (m, 6H), 0.96-0.44 (m, 3H). |
| 47 | 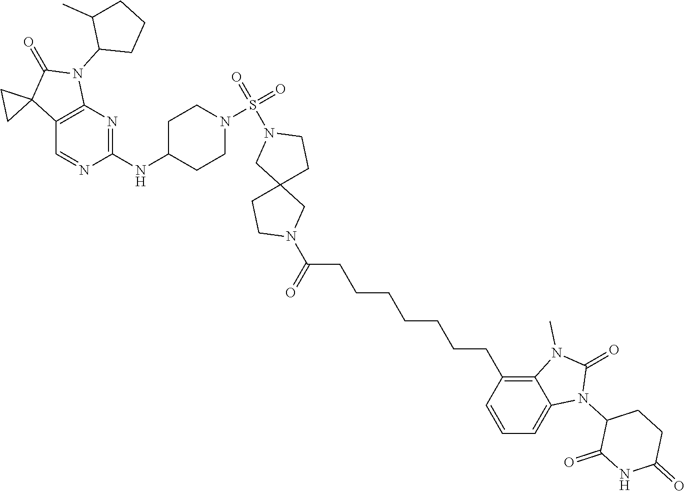 [SMILES: CC1CCCC1N1C(=O)C2(CC2)c2cnc(NC3CCN(S(=O)(=O)N4CCC5(CCN(C(=O)CCCCCCCc6cccc7c6n(C)c(=O)n7C6CCC(=O)NC6=O)C5)C4)CC3)nc21] | 913.4 | ¹H NMR (400 MHz, DMSO-d₆) δ 11.01 (s, 1H), 7.69 (d, J = 3.7 Hz, 1H), 7.16-6.30 (m, 4H), 5.29 (dd, J = 12.6, 5.4 Hz, 1H), 4.83-4.03 (m, 1H), 3.74 (s, 1H), 3.61-3.38 (m, 8H), 3.19- 3.03 (m, 4H), 2.88-2.73 (m, 5H), 2.70-2.51 (m, 3H), 2.14 (q, J = 7.8 Hz, 3H), 1.97-1.67 (m, 10H), 1.66-1.09 (m, 19H), 0.92-0.43 (m, 3H). |
| 48 | 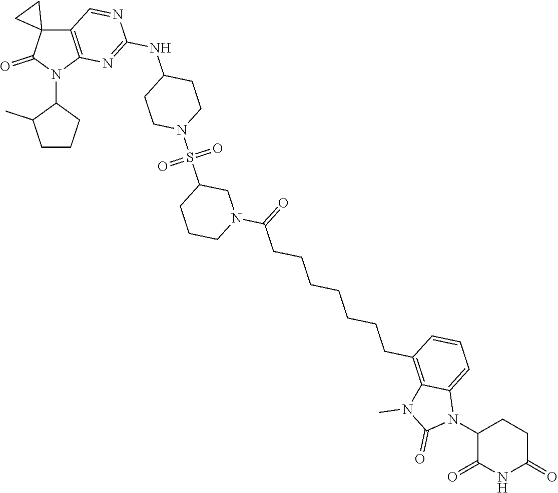 [SMILES: CC1CCCC1N1C(=O)C2(CC2)c2cnc(NC3CCN(S(=O)(=O)C4CCCN(C(=O)CCCCCCCc5cccc6c5n(C)c(=O)n6C5CCC(=O)NC5=O)C4)CC3)nc21] | 872.3 | ¹H NMR (400 MHz, DMSO-d₆) δ 11.08 (s, 1H), 7.76 (d, J = 3.6 Hz, 1H), 7.08 (s, 1H), 7.01- 6.89 (m, 2H), 6.88-6.79 (m, 1H), 5.35 (dd, J = 12.2, 5.3 Hz, 1H), 4.87-4.53 (m, 1H), 4.33- 4.10 (m, 1H), 4.05-3.73 (m, 2H), 3.72-3.60 (m, 2H), 3.54 (d, J = 5.5 Hz, 3H), 3.23-3.09 (m, 2H), 3.07-2.82 (m, 5H), 2.78-2.56 (m, 4H), 2.39-2.14 (m, 3H), 2.14-2.03 (m, 1H), 2.04-1.64 (m, 9H), 1.63-1.15 (m, 18H), 1.01 0.58 (m, 3H). |
| 49 | 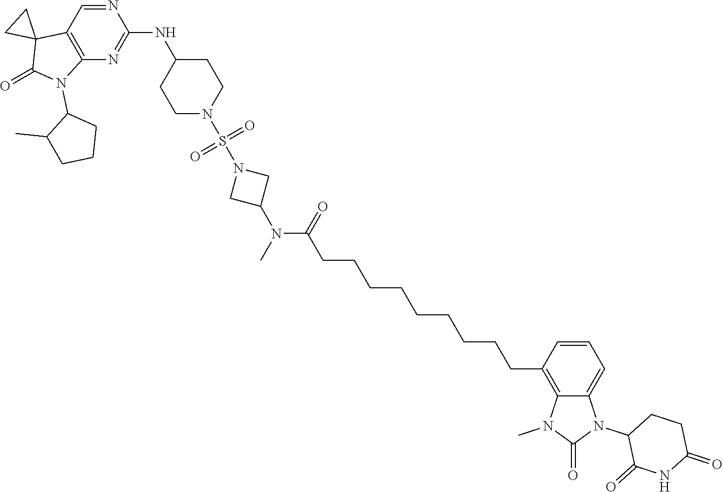 [SMILES: CC1CCCC1N1C(=O)C2(CC2)c2cnc(NC3CCN(S(=O)(=O)N4CC(N(C)C(=O)CCCCCCCCCc5cccc6c5n(C)c(=O)n6C5CCC(=O)NC5=O)C4)CC3)nc21] | 901.4 | ¹H NMR (400 MHz, DMSO-d₆) δ 11.08 (s, 1H), 7.76 (d, J = 3.4 Hz, 1H), 7.14-6.74 (m, 4H), 5.36 (dd, J = 12.6, 5.4 Hz, 1H), 5.13-4.47 (m, 1H), 4.30-3.70 (m, 6H), 3.65-3.45 (m, 5H), 3.04-2.81 (m, 8H), 2.77-2.53 (m, 4H), 2.37- 2.25 (m, 2H), 2.25-2.09 (m, 1H), 2.11-1.75 (m, 6H), 1.75-1.65 (m, 1H), 1.65-1.40 (m, 11H), 1.42-1.12 (m, 10H), 0.91-0.46 (m, 3H). |
| 50 | 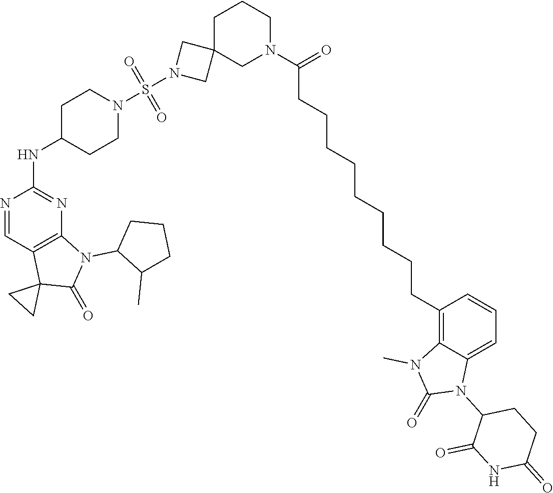 [SMILES: CC1CCCC1N1C(=O)C2(CC2)c2cnc(NC3CCN(S(=O)(=O)N4CC5(CCCN(C(=O)CCCCCCCCCc6cccc7c6n(C)c(=O)n7C6CCC(=O)NC6=O)C5)C4)CC3)nc21] | 942.5 | ¹H NMR (400 MHz, DMSO-d₆) δ 11.08 (s, 1H), 7.76 (d, J = 3.8 Hz, 1H), 7.19-6.91 (m, 3H), 6.91-6.75 (m, 1H), 5.36 (dd, J = 12.5, 5.4 Hz, 1H), 4.85-4.11 (m, 1H), 3.81 (m, 1H), 3.65-3.42 (m, 11H), 2.98-2.79 (m, 5H), 2.77-2.56 (m, 3H), 2.38-2.14 (m, 3H), 2.05-1.78 (m, 6H), 1.73 (s, 3H), 1.64-1.40 (m, 12H), 1.26 (m, 13H), 0.87 (d, J = 6.6 Hz, 2H), 0.73 (d, J = 7.0 Hz, 1H). |
| 51 | 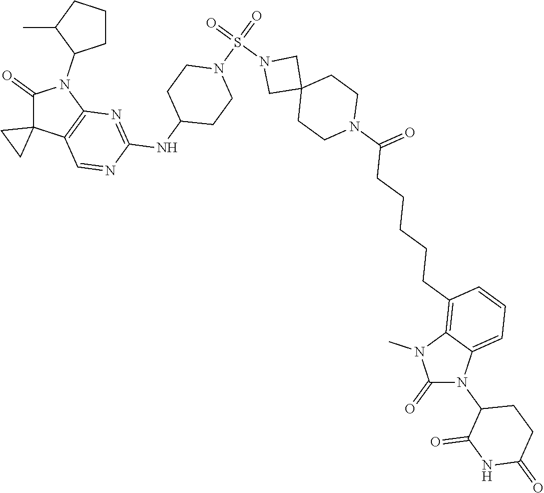 [SMILES: CC1CCCC1N1C(=O)C2(CC2)c2cnc(NC3CCN(S(=O)(=O)N4CC5(CCN(C(=O)CCCCCc6cccc7c6n(C)c(=O)n7C6CCC(=O)NC6=O)CC5)C4)CC3)nc21] | 941.5 | ¹H NMR (400 MHz, DMSO-d₆) δ 7.76 (d, J = 3.5 Hz, 1H), 7.08 (s, 1H), 7.00-6.90 (m, 2H), 6.86 (m, 1H), 5.36 (m, 1H), 4.81-4.15 (m, 1H), 3.77 (m, 1H), 3.66-3.49 (m, 9H), 3.38 (m, 3H), 2.99-2.81 (m, 5H), 2.79-2.56 (m, 3H), 2.38-2.14 (m, 3H), 2.05-1.75 (m, 6H), 1.75-1.41 (m, 15H), 1.40-1.15 (m, 12H), 0.88 (d, J = 6.8 Hz, 2H), 0.74 (d, J = 6.8 Hz, 1H). |
| 52 | 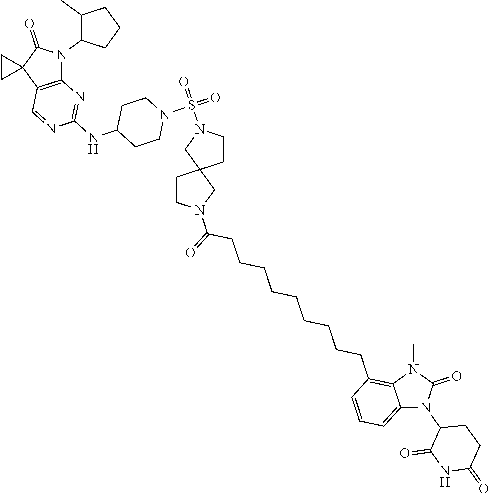 [SMILES: CC1CCCC1N1C(=O)C2(CC2)c2cnc(NC3CCN(S(=O)(=O)N4CCC5(CCN(C(=O)CCCCCCCCCc6cccc7c6n(C)c(=O)n7C6CCC(=O)NC6=O)C5)C4)CC3)nc21] | 941.4 | ¹H NMR (400 MHz, DMSO-d₆) δ 11.04 (s, 1H), 7.76 (dd, J = 3.6, 2.0 Hz, 1H), 7.07 (s, 1H), 6.99- 6.91 (m, 2H), 6.85 (m, 1H), 5.36 (dd, J = 12.6, 5.4 Hz, 1H), 4.83-4.12 (m, 1H), 3.81 (s, 1H), 3.66-3.45 (m, 6H), 3.38 (m, 2H), 3.25-3.09 (m, 3H), 2.89 (m, 5H), 2.77-2.57 (m, 3H), 2.20 (m, 3H), 2.04-1.63 (m, 12H), 1.64-1.41 (m, 11H), 1.41-1.17 (m, 11H), 0.88 (d, J = 6.8 Hz, 2H), 0.74 (d, J = 6.8 Hz, 1H). |
| 53 | 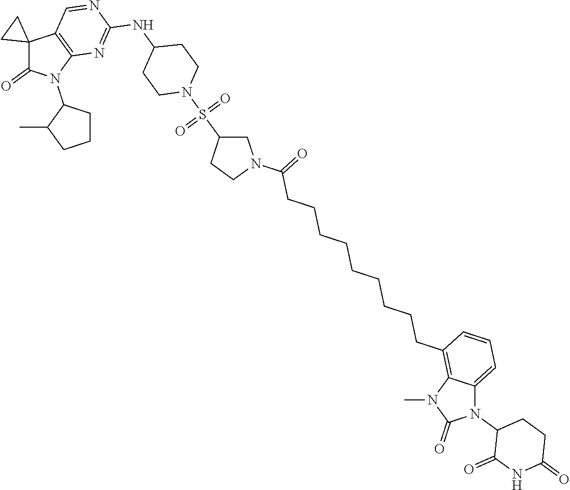 [SMILES: CC1CCCC1N1C(=O)C2(CC2)c2cnc(NC3CCN(S(=O)(=O)C4CCN(C(=O)CCCCCCCCCc5cccc6c5n(C)c(=O)n6C5CCC(=O)NC5=O)C4)CC3)nc21] | 886.5 | ¹H NMR (400 MHz, DMSO-d₆) δ 11.07 (s, 1H), 7.76 (d, J = 3.5 Hz, 1H), 7.08 (s, 1H), 7.01-6.90 (m, 2H), 6.90-6.84 (m, 1H), 5.35 (dd, J = 12.5, 5.4 Hz, 1H), 4.48 (m, 1H), 4.02 (m, 3H), 3.73- 3.59 (m, 3H), 3.54 (d, 1.82 Hz, 3H), 3.51-3.39 (m, 2H), 3.01 (d, J = 10.5 Hz, 3H), 2.89 (m, 3H), 2.76-2.57 (m, 3H), 2.54 (s, 1H), 2.36-2.15 (m, 5H), 2.13-1.77 (m, 7H), 1.73-1.42 (m, 10H), 1.37 (m, 3H), 1.27 (m, 5H), 1.24 (m, 1H), 0.88 (d, J = 6.8 Hz, 2H), 0.74 (d, J = 6.8 Hz, 1H). |
| 54 | 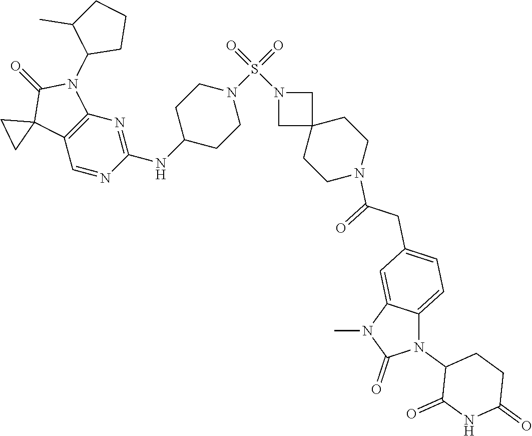 [SMILES: CC1CCCC1N1C(=O)C2(CC2)c2cnc(NC3CCN(S(=O)(=O)N4CC5(CCN(C(=O)Cc6ccc7c(c6)n(C)c(=O)n7C6CCC(=O)NC6=O)CC5)C4)CC3)nc21] | 829.4 | ¹H NMR (400 MHz, DMSO-d₆) δ 11.10 (s, 1H), 7.76 (d, J = 3.5 Hz, 1H), 7.31-6.77 (m, 4H), 5.35 (dd, J = 12.8, 5.4 Hz, 1H), 4.47 (dd, J = 234.1, 8.4 Hz, 1H), 3.73 (s, 3H), 3.50 (d, J = 52.0 Hz, 11H), 3.32 (s, 2H), 2.90 (t, J = 12.2 Hz, 3H), 2.79-2.58 (m, 3H), 2.19 (d, J = 10.2 Hz, 1H), 2.06-1.76 (m, 6H), 1.73-1.50 (m, 7H), 1.50-1.39 (m, 4H), 1.25 (d, J = 10.1 Hz, 1H), 0.96-0.70 (m, 3H) |
| 55 | 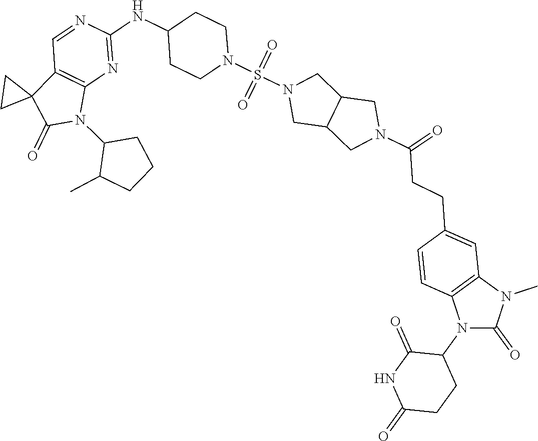 [SMILES: CC1CCCC1N1C(=O)C2(CC2)c2cnc(NC3CCN(S(=O)(=O)N4CC5CN(C(=O)CCc6ccc7c(c6)n(C)c(=O)n7C6CCC(=O)NC6=O)CC5C4)CC3)nc21] | 829.4 | ¹H NMR (400 MHz, DMSO-d₆) δ 11.01 (s, 1H), 7.79-7.28 (m, 2H), 7.14-6.67 (m, 3H), 5.26 (dd, J = 12.8, 5.3 Hz, 1H), 4.91-4.01 (m, 1H), 3.77 (s, 1H), 3.60-3.45 (m, 4H), 3.41-3.31 (m, 3H), 3.24 (s, 3H), 3.19-3.12 (m, 2H), 3.01 (ddd, J = 11.4, 7.8, 4.4 Hz, 2H), 2.91-2.73 (m, 7H), 2.68-2.55 (m, 2H), 2.56-2.44 (m, 2H), 2.19-2.04 (m, 1H), 1.96-1.68 (m, 6H), 1.67- 1.53 (m, 3H), 1.52-1.38 (m, 4H), 1.26-1.09 (m, 1H), 0.92-0.33 (m, 3H). |
| 56 | 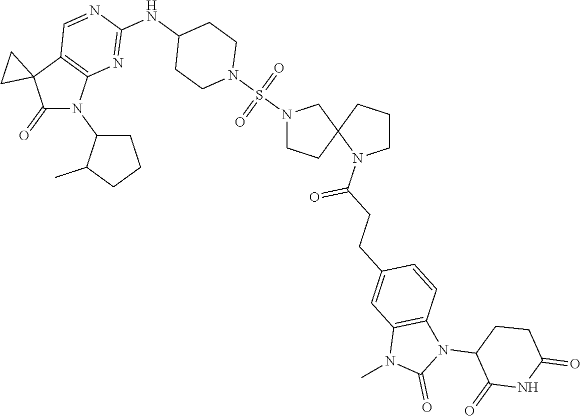 [SMILES: CC1CCCC1N1C(=O)C2(CC2)c2cnc(NC3CCN(S(=O)(=O)N4CCC5(CCCN5C(=O)CCc5ccc6c(c5)n(C)c(=O)n6C5CCC(=O)NC5=O)C4)CC3)nc21] | 843.4 | ¹H NMR (400 MHz, DMSO-d₆) δ 11.08 (s, 1H), 7.76 (d, J = 3.8 Hz, 1H), 7.30-6.35 (m, 4H), 5.33 (dd, J = 12.7, 5.4 Hz, 1H), 4.87-4.05 (m, 1H), 3.87-3.72 (m, 2H), 3.66-3.55 (m, 2H), 3.53-3.37 (m, 4H), 3.31 (s, 4H), 3.31-3.20 (m, 1H), 2.98-2.79 (m, 7H), 2.71 (td, J = 13.0, 4.2 Hz, 1H), 2.65-2.61 (m, 1H), 2.62-2.52 (m, 2H), 2.29-2.12 (m, 1H), 2.03-1.89 (m, 5H), 1.89-1.81 (m, 1H), 1.82-1.69 (m, 3H), 1.68-1.47 (m, 6H), 1.49-1.40 (m, 2H), 1.23 (t, J = 10.2 Hz, 1H), 0.94-0.62 (m, 3H). |
| 57 | 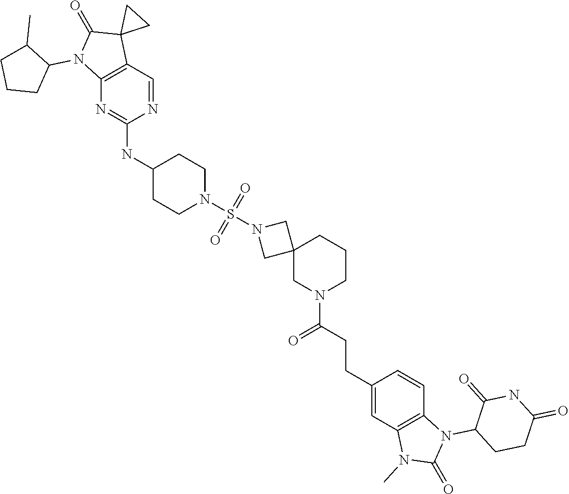 [SMILES: CC1CCCC1N1C(=O)C2(CC2)c2cnc(NC3CCN(S(=O)(=O)N4CC5(CCCN(C(=O)CCc6ccc7c(c6)n(C)c(=O)n7C6CCC(=O)NC6=O)C5)C4)CC3)nc21] | 843.3 | ¹H NMR (400 MHz, DMSO-d₆) δ 11.09 (s, 1H), 7.88-7.65 (m, 2H), 7.08-6.94 (m, 1H), 7.03- 6.94 (m, 1H), 6.92 (dd, J = 8.1, 1.5 Hz, 1H), 5.33 (dt, J = 13.8, 6.7 Hz, 1H), 4.81-4.12 (m, 1H), 3.92-3.74 (m, 1H), 3.69-3.46 (m, 6H), 3.43 (s, 4H), 3.31 (s, 1H), 3.05-2.79 (m, 5H), 2.77-2.56 (m, 5H), 2.20 (s, 1H), 2.06-1.77 (m, 6H), 1.77- 1.64 (m, 4H), 1.62-1.40 (m, 7H), 1.41-1.31 (m, 2H), 1.29-1.17 (m, 1H), 0.87 (t, J = 7.2 Hz, 2H), 0.73 (t, J = 7.2 Hz, 1H). |
| 58 | 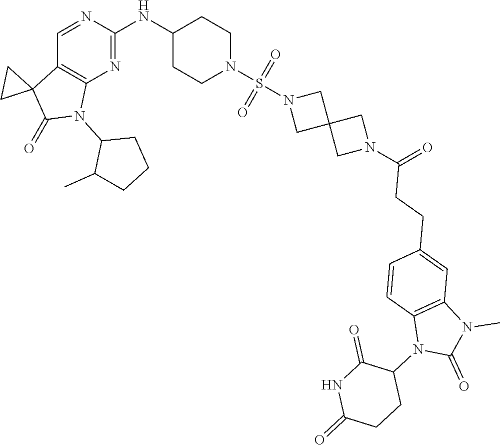 [SMILES: CC1CCCC1N1C(=O)C2(CC2)c2cnc(NC3CCN(S(=O)(=O)N4CC5(CN(C(=O)CCc6ccc7c(c6)n(C)c(=O)n7C6CCC(=O)NC6=O)C5)C4)CC3)nc21] | 815.5 | ¹H NMR (400 MHz, DMSO-d₆) δ 11.08 (s, 1H), 7.76 (d, J = 3.6 Hz, 1H), 7.14-6.95 (m, 3H), 6.88 (dd, J = 8.1, 1.6 Hz, 1H), 5.34 (dd, J = 12.8, 5.4 Hz, 1H), 4.86-4.15 (m, 1H), 4.17-4.04 (m, 2H), 3.97 (s, 2H), 3.93-3.73 (m, 5H), 3.63- 3.49 (m, 2H), 2.96-2.70 (m, 6H), 2.68-2.56 (m, 3H), 2.32 (t, J = 7.6 Hz, 2H), 2.26-2.12 (m, 1H), 2.04-1.74 (m, 7H), 1.68 (s, 1H), 1.60- 1.53 (m, 2H), 1.53-1.36 (m, 5H), 1.25 (q, J = 10.0 Hz, 1H), 0.94-0.64 (m, 3H). |
| 59 | 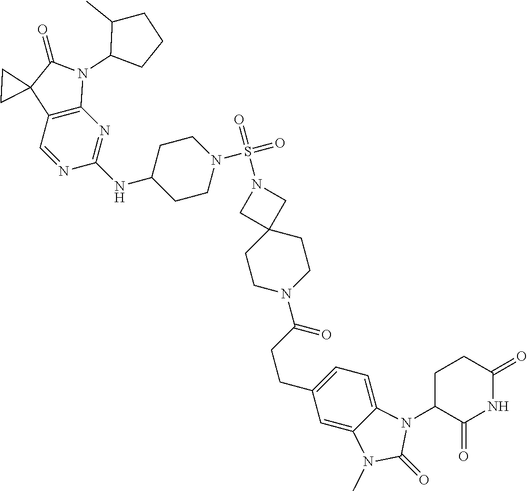 [SMILES: CC1CCCC1N1C(=O)C2(CC2)c2cnc(NC3CCN(S(=O)(=O)N4CC5(CCN(C(=O)CCc6ccc7c(c6)n(C)c(=O)n7C6CCC(=O)NC6=O)CC5)C4)CC3)nc21] | 843.4 | ¹H NMR (400 MHz, DMSO-d₆) δ 11.08 (s, 1H), 7.76 (d, J = 3.4 Hz, 1H), 7.08 (s, 1H), 6.99 (d, J = 8.0 Hz, 1H), 6.90 (d, J = 8.0 Hz, 1H), 5.34 (dd, J = 12.6, 5.4 Hz, 1H), 4.83-4.10 (m, 1H), 3.81 (s, 1H), 3.56 (s, 6H), 3.40 (s, 2H), 3.38-3.34 (m, 2H), 2.97-2.78 (m, 5H), 2.76-2.56 (m, 5H), 2.30- 2.10 (m, 1H), 2.03-1.74 (m, 6H), 1.73-1.65 (m, 1H) 1.63-1.39 (m, 11H), 1.24 (s, 1H), 0.88 (d, J = 6.6 Hz, 2H), 0.74 (d, J = 6.9 Hz, 1H). |
| 60 | 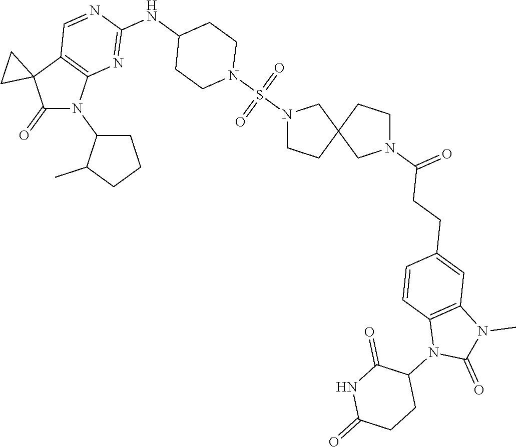 [SMILES: CC1CCCC1N1C(=O)C2(CC2)c2cnc(NC3CCN(S(=O)(=O)N4CCC5(CCN(C(=O)CCc6ccc7c(c6)n(C)c(=O)n7C6CCC(=O)NC6=O)C5)C4)CC3)nc21] | 843.4 | ¹H NMR (400 MHz, DMSO-d₆) δ 11.03 (s, 1H), 7.76 (t, J = 3.4 Hz, 1H), 7.32-6.51 (m, 4H), 5.32 (dd, J = 12.7, 5.4 Hz, 1H), 4.89-4.09 (m, 1H), 3.80 (s, 1H), 3.56 (d, J = 11.8 Hz, 2H), 3.45 (t, J = 7.2 Hz, 1H), 3.41-3.33 (m, 2H), 3.29- 3.09 (m, 5H), 2.97-2.81 (m, 5H), 2.77-2.54 (m, 6H), 2.28-2.14 (m, 2H), 2.05-1.74 (m, 11H), 1.69 (s, 1H), 1.61-1.38 (m, 6H), 1.30- 1.16 (m, 1H), 0.94-0.83 (m, 2H), 0.80-0.58 (m, 1H). |
| 61 | 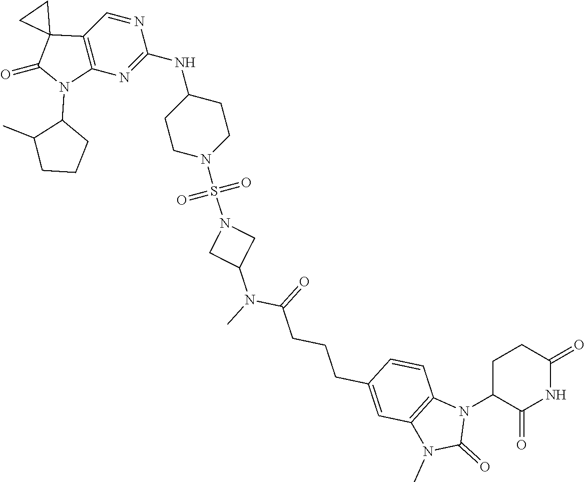 [SMILES: CC1CCCC1N1C(=O)C2(CC2)c2cnc(NC3CCN(S(=O)(=O)N4CC(N(C)C(=O)CCCc5ccc6c(c5)n(C)c(=O)n6C5CCC(=O)NC5=O)C4)CC3)nc21] | 817.4 | ¹H NMR (400 MHz, DMSO-d₆) δ 11.09 (s, 1H), 7.76 (d, J = 3.6 Hz, 1H), 7.20-6.83 (m, 4H), 5.41-5.28 (m, 1H), 5.10-4.74 (m, 1H), 4.27- 3.74 (m, 6H), 3.62-3.50 (m, 2H), 3.28-3.09 (m, 1H), 3.04-2.82 (m, 6H), 2.77-2.59 (m, 5H), 2.38-1.64 (m, 12H), 1.60-1.41 (m, 7H), 1.29-1.17 (m, 2H), 0.92-0.67 (m, 3H). |
| 62 | 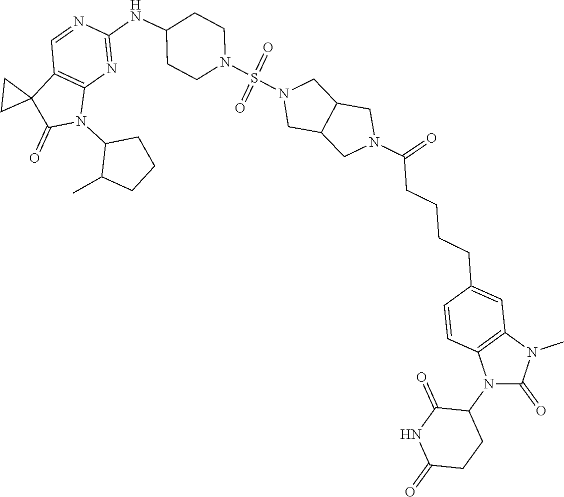 [SMILES: CC1CCCC1N1C(=O)C2(CC2)c2cnc(NC3CCN(S(=O)(=O)N4CC5CN(C(=O)CCCCc6ccc7c(c6)n(C)c(=O)n7C6CCC(=O)NC6=O)CC5C4)CC3)nc21] | 843.3 | ¹H NMR (400 MHz, DMSO-d₆) δ 11.07 (s, 1H), 7.86-7.63 (m, 1H), 7.21-6.72 (m, 4H), 5.33 (dd, J = 12.8, 5.4 Hz, 1H), 4.87-4.06 (m, 1H), 3.80 (s, 1H), 3.72-3.34 (m, 7H), 3.31 (s, 2H), 3.29-3.14 (m, 2H), 3.15-3.00 (m, 2H), 3.00- 2.77 (m, 5H), 2.76-2.53 (m, 5H), 2.34-2.11 (m, 3H), 2.05-1.74 (m, 7H), 1.73-1.62 (m, 1H), 1.60-1.36 (m, 6H), 1.31-1.16 (m, 2H), 0.95-0.65 (m, 3H). |
| 63 | 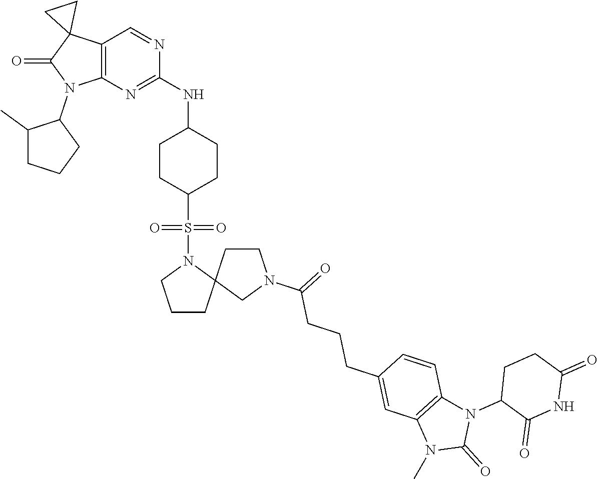 [SMILES: CC1CCCC1N1C(=O)C2(CC2)c2cnc(NC3CCC(S(=O)(=O)N4CCCC45CCN(C(=O)CCCc4ccc6c(c4)n(C)c(=O)n6C4CCC(=O)NC4=O)C5)CC3)nc21] | 857.4 | ¹H NMR (400 MHz, DMSO-d₆) δ 11.08 (s, 1H), 7.76 (d, J = 3.6 Hz, 1H), 7.22-6.77 (m, 4H), 5.33 (dd, J = 12.7, 5.4 Hz, 1H), 4.85-4.00 (m, 1H), 3.90-3.66 (m, 1H), 3.65-3.47 (m, 4H), 3.48-3.36 (m, 1H), 3.27-3.12 (m, 2H), 2.99- 2.80 (m, 3H), 2.77-2.55 (m, 4H), 2.31-2.12 (m, 2H), 2.04-1.61 (m, 13H), 1.62-1.37 (m, 8H), 1.35-1.07 (m, 5H), 0.99-0.79 (m, 4H), 0.74 (d, J = 6.9 Hz, 1H). |
| 64 | 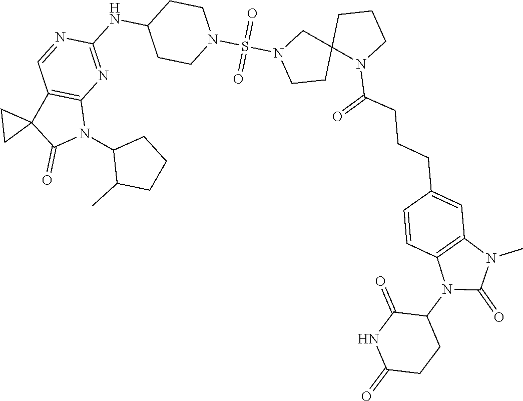 [SMILES: CC1CCCC1N1C(=O)C2(CC2)c2cnc(NC3CCN(S(=O)(=O)N4CCC5(CCCN5C(=O)CCCc5ccc6c(c5)n(C)c(=O)n6C5CCC(=O)NC5=O)C4)CC3)nc21] | 857.4 | ¹H NMR (400 MHz, DMSO-d6) δ 11.00 (s, 1H), 7.69 (dd, J = 4.8, 3.7 Hz, 1H), 7.14-6.69 (m, 4H), 5.26 (dd, J = 12.7, 5.4 Hz, 1H), 4.81-4.02 (m, 1H), 3.82-3.65 (m, 2H), 3.52 (s, 2H), 3.51- 3.37 (m, 3H), 3.19-3.09 (m, 3H), 3.19-3.09 (m, 2H), 2.91-2.71 (m, 5H), 2.69-2.51 (m, 4H), 2.25-2.16 (m, 1H), 2.25-2.06 (m, 1H), 2.01 (s, 1H), 1.97-1.81 (m, 6H), 1.81-1.63 (m, 6H), 1.60-1.54 (m, 1H), 1.51-1.29 (m, 7H), 1.25-1.20 (m, 1H), (t, J = 6.3 Hz, 2H), 0.89- 0.54 (m, 3H). |
| 65 | 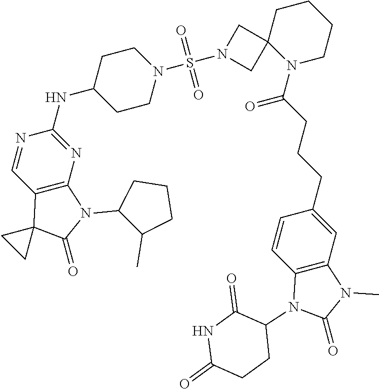 [SMILES: CC1CCCC1N1C(=O)C2(CC2)c2cnc(NC3CCN(S(=O)(=O)N4CC5(CCCCN5C(=O)CCCc5ccc6c(c5)n(C)c(=O)n6C5CCC(=O)NC5=O)C4)CC3)nc21] | 857.3 | ¹H NMR (400 MHz, DMSO-d₆) δ 11.07 (s, 1H), 7.76 (dd, J = 3.5, 1.0 Hz, 1H), 7.22-6.67 (m, 4H), 5.33 (dd, J = 12.2, 5.0 Hz, 1H), 4.86-4.08 (m, 1H), 3.87-3.72 (m, 3H), 3.72-3.61 (m, 2H), 3.60-3.48 (m, 2H), 3.34 (s, 2H), 3.28- 3.12 (m, 2H), 3.01-2.80 (m, 3H), 2.78-2.55 (m, 5H), 2.32 (t, J = 7.3 Hz, 1H), 2.25-2.12 (m, 1H), 2.04-1.61 (m, 13H), 1.61-1.40 (m, 7H), 1.38-1.13 (m, 4H), 0.88 (d, J = 6.6 Hz, 2H), 0.73 (d, J = 6.9 Hz, 1H). |
| 66 | 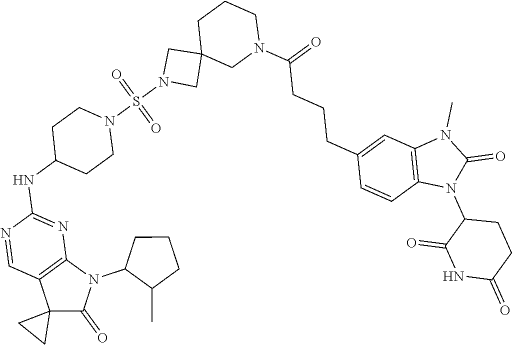 [SMILES: CC1CCCC1N1C(=O)C2(CC2)c2cnc(NC3CCN(S(=O)(=O)N4CC5(CCCN(C(=O)CCCc6ccc7c(c6)n(C)c(=O)n7C6CCC(=O)NC6=O)C5)C4)CC3)nc21] | 857.3 | ¹H NMR (400 MHz, DMSO-d₆) δ 11.08 (s, 1H), 7.76 (t, J = 3.6 Hz, 1H), 7.16-6.96 (m, 3H), 6.96- 6.83 (m, 1H), 5.40-5.26 (m, 1H), 4.82-4.07 (m, 1H), 3.81 (s, 1H), 3.75-3.37 (m, 9H), 3.31-3.18 (m, 3H), 2.90 (t, J = 11.9 Hz, 3H), 2.77-2.55 (m, 4H), 2.54 (s, 1H), 2.44-2.28 (m, 1H), 2.21 (m, 1H), 1.82 (m, 10H), 1.62-1.33 (m, 8H), 1.24 (d, J = 7.0 Hz, 4H), 0.88 (d, J = 6.8, 2H), 0.73 (d, J = 7.0, 1H). |
| 67 | 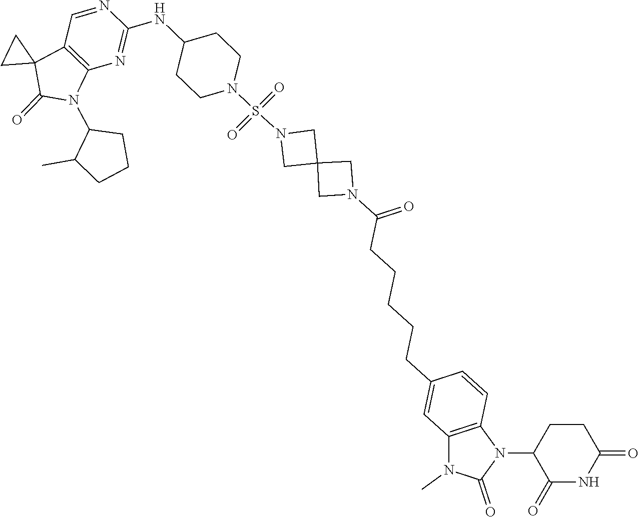 [SMILES: CC1CCCC1N1C(=O)C2(CC2)c2cnc(NC3CCN(S(=O)(=O)N4CC5(CN(C(=O)CCCCCc6ccc7c(c6)n(C)c(=O)n7C6CCC(=O)NC6=O)C5)C4)CC3)nc21] | 829.4 | ¹H NMR (400 MHz, DMSO-d6) δ 11.08 (s, 1H), 7.76 (d, J = 3.5 Hz, 1H), 7.16-6.78 (m, 4H), 5.34 (dd, J = 12.7, 5.3 Hz, 1H), 4.84-4.12 (m, 3H), 4.08-3.69 (m, 7H), 3.55 (s, 2H), 3.36- 3.32 (m, 3H), 3.18 (q, J = 7.0 Hz, 1H), 2.96- 2.84 (m, 3H), 2.77-2.56 (m, 4H), 2.25-2.26 (m, 1H), 2.25-2.15 (m, 1H), 2.10-1.76 (m, 8H), 1.69 (s, 1H), 1.60-1.41 (m, 6H), 1.23 (dd, J = 6.8, 1.7 Hz, 2H), 0.88 (d, J = 6.6 Hz, 2H), 0.74 (d, J = 7.0 Hz, 1H). |
| 68 | 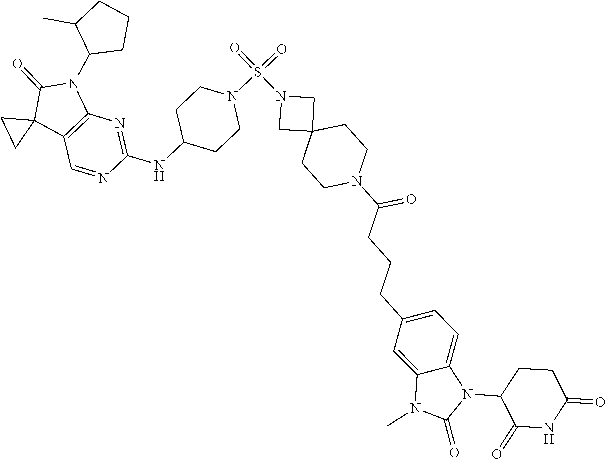 [SMILES: CC1CCCC1N1C(=O)C2(CC2)c2cnc(NC3CCN(S(=O)(=O)N4CC5(CCN(C(=O)CCCc6ccc7c(c6)n(C)c(=O)n7C6CCC(=O)NC6=O)CC5)C4)CC3)nc21] | 857.4 | ¹H NMR (400 MHz, DMSO-d₆) δ 11.08 (s, 1H), 7.76 (d, J = 3.5 Hz, 1H), 7.25-6.81 (m, 4H), 5.34 (dd, J = 12.7, 5.4 Hz, 1H), 4.83-4.09 (m, 1H), 3.81 (s, 1H), 3.63-3.48 (m, 6H), 3.46-3.37 (m, 2H), 3.34 (s, 2H), 3.32-3.18 (m, 2H), 2.98-2.85 (m, 3H), 2.77-2.55 (m, 5H), 2.32 (t, J = 7.3 Hz, 1H), 2.21 (s, 1H), 2.04-1.75 (m, 7H), 1.74-1.37 (m, 12H), 1.23 (d, J = 6.7 Hz, 3H), 0.88 (d, J = 6.6 Hz, 2H), 0.74 (d, J = 7.0 Hz, 1H). |
| 69 | 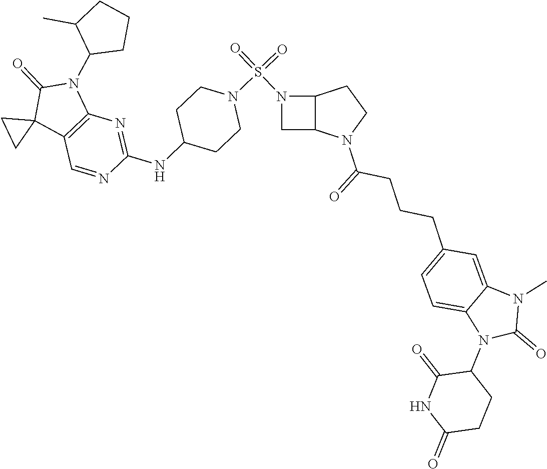 [SMILES: CC1CCCC1N1C(=O)C2(CC2)c2cnc(NC3CCN(S(=O)(=O)N4CC5C4CCN5C(=O)CCCc4ccc5c(c4)n(C)c(=O)n5C4CCC(=O)NC4=O)CC3)nc21] | 829.4 | ¹H NMR (400 MHz, DMSO-d₆) δ 11.09 (s, 1H), 8.13-7.54 (m, 1H), 7.35-6.79 (m, 4H), 5.34 (dd, J = 12.8, 5.2 Hz, 1H), 5.01-4.14 (m, 3H), 4.10-3.65 (m, 4H), 3.63-3.38 (m, 4H), 3.29- 3.01 (m, 1H), 3.01-2.78 (m, 3H), 2.76-2.57 (m, 5H), 2.43-2.03 (m, 4H), 2.03-1.61 (m, 10H), 1.60-1.33 (m, 6H), 1.30-1.13 (m, 2H), 1.02-0.22 (m, 3H). |
| 70 | 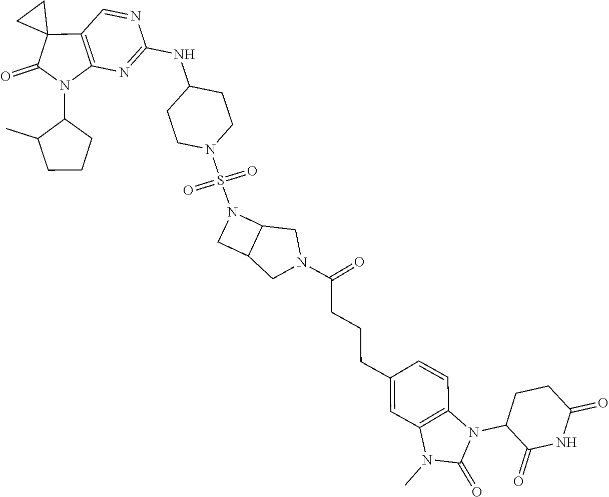 [SMILES: CC1CCCC1N1C(=O)C2(CC2)c2cnc(NC3CCN(S(=O)(=O)N4CC5CN(C(=O)CCCc6ccc7c(c6)n(C)c(=O)n7C6CCC(=O)NC6=O)CC54)CC3)nc21] | 829.5 | ¹H NMR (400 MHz, DMSO-d6) δ 11.08 (s, 1H), 7.80-7.67 (m, 1H), 7.24-6.76 (m, 4H), 5.27- 5.24 (m, 1H), 4.86-4.59 (m, 1H), 4.24-4.09 (m, 1H), 4.09-3.91 (m, 2H), 3.91-3.68 (m, 2H), 3.61-352 (m, 2H), 3.21-2.98 (m, 5H), 2.94-2.76 (m, 4H), 2.72-2.56 (m, 5H), 2.37- 2.14 (m, 2H), 2.07-1.74 (m, 8H), 1.67 (s, 1H), 1.56-1.31 (m, 7H), 1.29-1.06 (m, 2H), 0.79 (dd, J = 59.1, 6.8 Hz, 3H). |
| 71 | 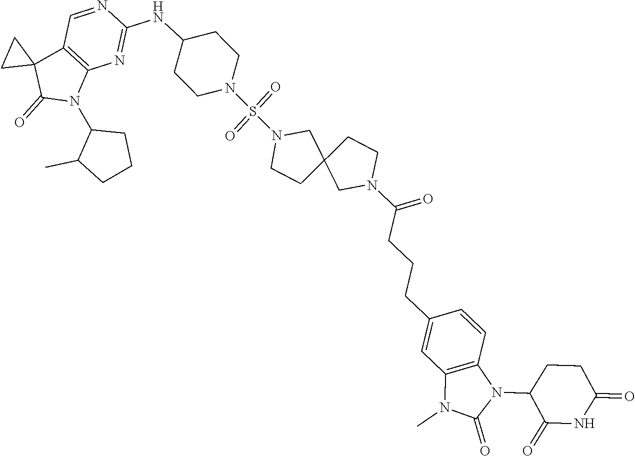 [SMILES: CC1CCCC1N1C(=O)C2(CC2)c2cnc(NC3CCN(S(=O)(=O)N4CCC5(CCN(C(=O)CCCc6ccc7c(c6)n(C)c(=O)n7C6CCC(=O)NC6=O)C5)C4)CC3)nc21] | 857.4 | ¹H NMR (400 MHz, DMSO-d₆) δ 11.09 (s, 1H), 7.91-7.64 (m, 1H), 7.19-6.96 (m, 3H), 6.96-6.78 (m, 1H), 5.34 (dd, J = 12.8, 5.4 Hz, 1H), 4.88- 4.11 (m, 1H), 3.81 (s, 1H), 3.65-3.51 (m, 2H), 3.51-3.44 (m, 1H), 3.38 (d, J = 8.8 Hz, 1H), 3.30-3.06 (m, 5H), 2.83-2.99 (m, 3H), 2.78-2.55 (m, 3H), 2.13-2.3 (m, 2H), 2.06-1.64 (m, 14H), 1.61-1.38 (m, 8H), 1.31-1.16 (m, 3H), 0.88 (d, J = 6.6 Hz, 2H), 0.74 (d, J = 7.0 Hz, 1H). |
| 72 | 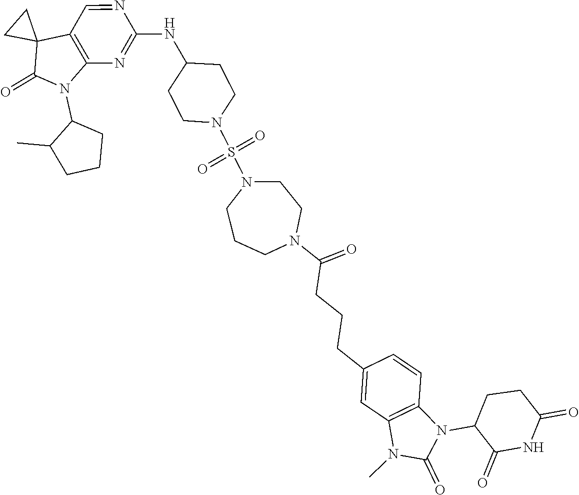 [SMILES: CC1CCCC1N1C(=O)C2(CC2)c2cnc(NC3CCN(S(=O)(=O)N4CCCN(C(=O)CCCc5ccc6c(c5)n(C)c(=O)n6C5CCC(=O)NC5=O)CC4)CC3)nc21] | 831.4 | ¹H NMR (400 MHz, DMSO-d6) δ 11.08 (s, 1H), 7.76 (d, J = 3.3 Hz, 1H), 7.20-6.77 (m, 4H), 5.33 (dd, J = 12.8, 5.4 Hz, 1H), 4.84-4.09 (m, 1H), 3.77 (s, 1H), 3.64-3.39 (m, 8H), 3.64- 3.39 (m, 1H), 2.98-2.74 (m, 4H), 2.74-2.56 (m, 5H), 2.42-2.30 (m, 1H), 2.28-2.14 (m, 1H), 2.07-1.72 (m, 8H), 1.71-1.57 (m, 3H), 1.55-1.35 (m, 7H), 1.24 (d, J = 6.3 Hz, 3H), 0.81 (dd, J = 57.6, 6.6 Hz, 4H). |
| 73 | 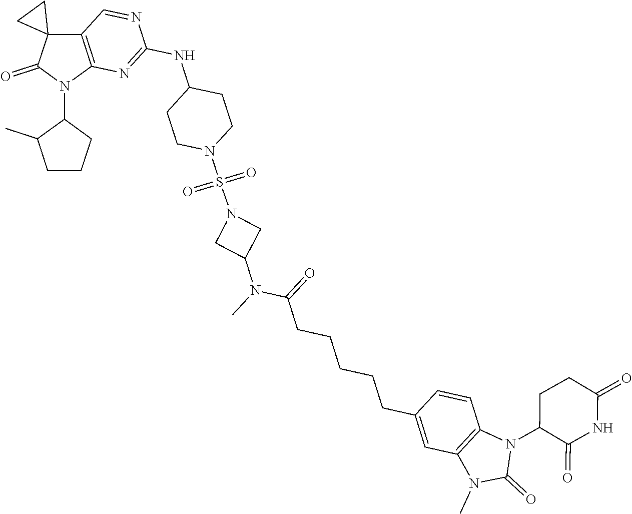 [SMILES: CC1CCCC1N1C(=O)C2(CC2)c2cnc(NC3CCN(S(=O)(=O)N4CC(N(C)C(=O)CCCCCc5ccc6c(c5)n(C)c(=O)n6C5CCC(=O)NC5=O)C4)CC3)nc21] | 845.3 | ¹H NMR (400 MHz, DMSO-d₆) δ 11.07 (s, 1H), 7.76 (d, J = 3.5 Hz, 1H), 7.16-6.92 (m, 3H), 6.86 (dd, J = 8.1, 1.5 Hz, 1H), 5.33 (dd, J = 12.7, 5.4 Hz, 1H), 5.05-4.70 (m, 1H), 4.25-4.12 (d, J = 9.0 Hz, 1H), 4.06-3.72 (m, 5H), 3.58 (d, J = 12.0 Hz, 2H), 3.03-2.80 (m, 6H), 2.76-2.53 (m, 5H), 2.39- 2.26 (m, 3H), 2.26-1.76 (m, 8H), 1.73-1.17 (m, 15H), 0.88 (d, J = 6.6 Hz, 2H), 0.74 (d, J = 7.0 Hz, 1H). |
| 74 | 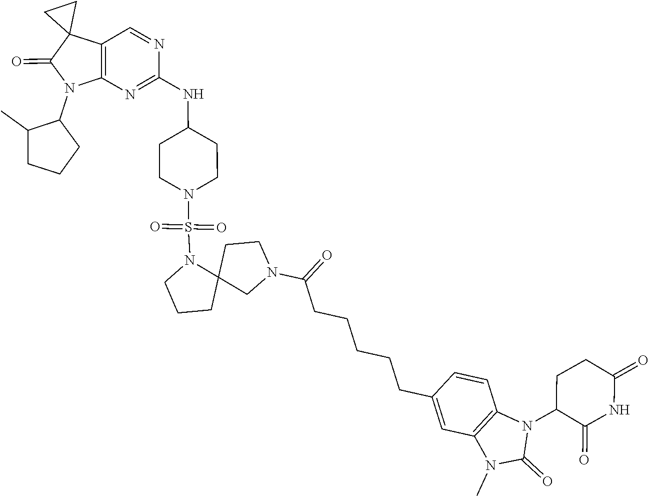 [SMILES: CC1CCCC1N1C(=O)C2(CC2)c2cnc(NC3CCN(S(=O)(=O)N4CCCC45CCN(C(=O)CCCCCc4ccc6c(c4)n(C)c(=O)n6C4CCC(=O)NC4=O)C5)CC3)nc21] | 885.4 | ¹H NMR (400 MHz, DMSO-d₆) δ 10.78 (s, 1H), 7.76 (d, J = 3.7 Hz, 1H), 7.15-6.95 (m, 3H), 6.86 (dt, J = 8.0, 1.8 Hz, 1H), 5.32 (dd, J = 12.7, 5.4 Hz, 1H), 4.87-4.12 (m, 1H), 3.76 (s, 1H), 3.66-3.47 (m, 5H), 3.40 (d, J = 9.8 Hz, 1H), 3.14 (dd, J = 11.9, 7.4 Hz, 1H), 2.96-2.81 (m, 3H), 2.76-2.53 (m, 6H), 2.31-2.14 (m, 4H), 2.05-1.74 (m, 13H), 1.64-1.38 (m, 11H), 1.35- 1.16 (m, 4H), 0.88 (d, J = 6.6 Hz, 2H), 0.74 (d, J = 7.0 Hz, 1H). |
| 75 | 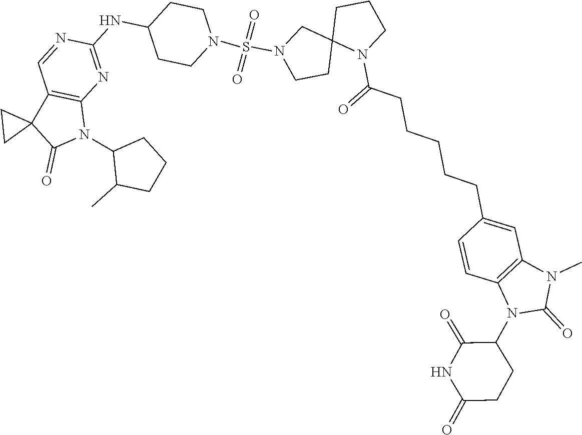 [SMILES: CC1CCCC1N1C(=O)C2(CC2)c2cnc(NC3CCN(S(=O)(=O)N4CCC5(CCCN5C(=O)CCCCCc5ccc6c(c5)n(C)c(=O)n6C5CCC(=O)NC5=O)C4)CC3)nc21] | 885.3 | ¹H NMR (400 MHz, DMSO-d₆) δ 11.01 (s, 1H), 7.68 (d, J = 3.7 Hz, 1H), 6.94 (dt, J = 16.3, 8.0 Hz, 3H), 6.78 (d, J = 8.0 Hz, 1H), 5.26 (dd, J = 12.9, 5.2 Hz, 1H), 4.80-4.00 (m, 1H), 3.82-3.61 (m, 2H), 3.52 (d, J = 11.7 Hz, 2H), 3.46-3.37 (m, 3H), 3.25 (s, 3H), 3.17 (m, 2H), 2.83 (m, 4H), 2.77-2.59 (m, 2H), 2.59-2.50 (m, 3H), 2.22-2.10 (m, 3H), 1.98-1.66 (m, 10H), 1.62-1.31 (m, 12H), 1.31-1.10 (m, 3H), 0.74 (dd, J = 57.8, 6.7 Hz, 3H). |
| 76 | 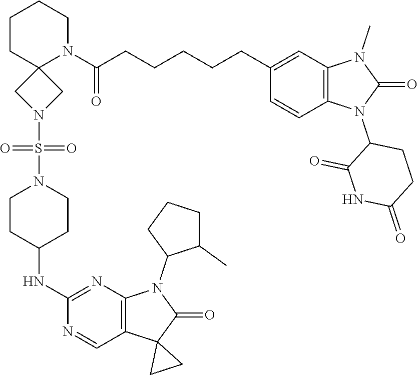 [SMILES: CC1CCCC1N1C(=O)C2(CC2)c2cnc(NC3CCN(S(=O)(=O)N4CC5(CCCCN5C(=O)CCCCCc5ccc6c(c5)n(C)c(=O)n6C5CCC(=O)NC5=O)C4)CC3)nc21] | 885.4 | ¹H NMR (400 MHz, DMSO-d₆) δ 11.07 (s, 1H), 7.76 (d, J = 3.5 Hz, 1H), 7.17-6.94 (m, 3H), 6.89-6.74 (m, 1H), 5.33 (dd, J = 12.7, 5.3 Hz, 1H), 4.88-4.02 (m, 1H), 3.81 (d, J = 8.2 Hz, 3H), 3.67 (d, J = 8.0 Hz, 2H), 3.56 (d, J = 11.9 Hz, 2H), 3.23 (s, 2H), 2.99-2.83 (m, 3H), 2.77- 2.55 (m, 5H), 2.35-2.14 (m, 4H), 2.03-1.74 (m, 9H), 1.73-1.39 (m, 15H), 1.39-1.09 (m, 4H), 0.88 (d, J = 6.6 Hz, 2H), 0.74 (d, J = 7.0 Hz, 1H). |
| 77 | 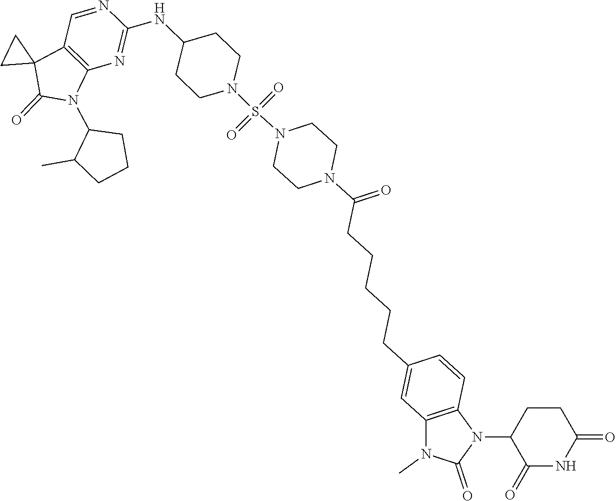 [SMILES: CC1CCCC1N1C(=O)C2(CC2)c2cnc(NC3CCN(S(=O)(=O)N4CCN(C(=O)CCCCCc5ccc6c(c5)n(C)c(=O)n6C5CCC(=O)NC5=O)CC4)CC3)nc21] | 845.3 | ¹H NMR (400 MHz, DMSO-d₆) δ 11.01 (s, 1H), 7.69 (d, J = 3.6 Hz, 1H), 7.18-6.87 (m, 3H), 6.79 (dd, J = 8.1, 1.6 Hz, 1H), 5.26 (dd, J = 12.8, 5.3 Hz, 1H), 4.76-4.00 (m, 1H), 3.76 (s, 1H), 3.47 (dd, J = 31.8, 8.8 Hz, 6H), 3.19-2.98 (m, 5H), 2.97-2.73 (m, 4H), 2.70-2.48 (m, 5H), 2.25 (t, J = 7.4 Hz, 2H), 2.11 (m, 1H), 1.99-1.68 (m, 6H), 1.66-1.31 (m, 12H), 1.31-1.01 (m, 3H), 0.74 (dd, J = 57.4, 6.8 Hz, 3H). |
| 78 | 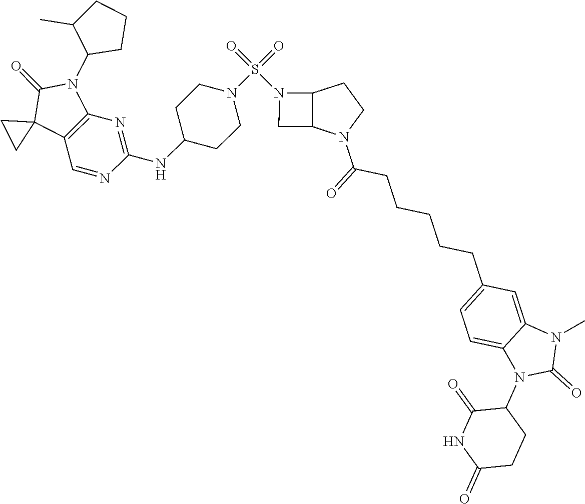 [SMILES: CC1CCCC1N1C(=O)C2(CC2)c2cnc(NC3CCN(S(=O)(=O)N4CC5C4CCN5C(=O)CCCCCc4ccc5c(c4)n(C)c(=O)n5C4CCC(=O)NC4=O)CC3)nc21] | 857.3 | ¹H NMR (400 MHz, DMSO-d₆) δ 11.07 (s, 1H), 7.76 (d, J = 3.4 Hz, 1H), 7.25-6.95 (m, 3H), 6.86 (d, J = 8.1 Hz, 1H), 5.33 (dd, J = 12.7, 5.4 Hz, 1H), 4.85 (dt, J = 39.3, 5.3 Hz, 1H), 4.59- 4.40 (m, 1H), 4.26-3.65 (m, 5H), 3.48 (dd, J = 36.0, 9.9 Hz, 4H), 3.26 (d, J = 10.9 Hz, 1H), 3.00-2.78 (m, 3H), 2.78-2.54 (m, 5H), 2.44- 2.11 (m, 3H), 2.11-1.40 (m, 20H), 1.39-1.15 (m, 3H), 0.88 (d, J = 6.6 Hz, 2H), 0.73 (d, J = 6.9 Hz, 1H). |
| 79 | 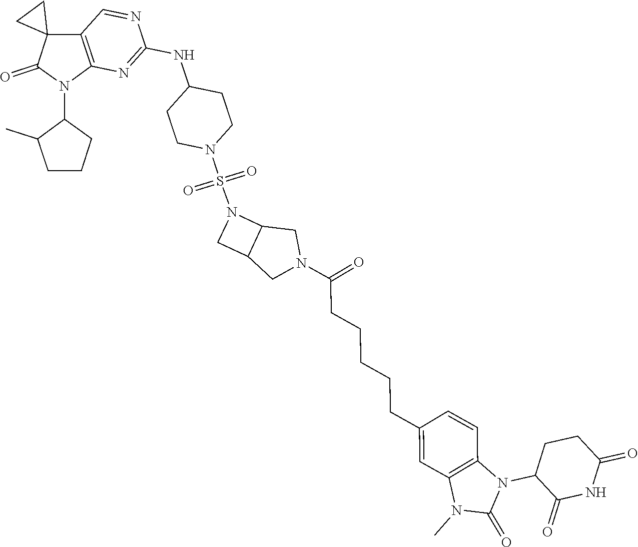 [SMILES: CC1CCCC1N1C(=O)C2(CC2)c2cnc(NC3CCN(S(=O)(=O)N4CC5CN(C(=O)CCCCCc6ccc7c(c6)n(C)c(=O)n7C6CCC(=O)NC6=O)CC54)CC3)nc21] | 857.4 | ¹H NMR (400 MHz, DMSO-d₆) δ 10.97 (s, 1H), 7.67 (s, 1H), 6.99-6.88 (m, 3H), 6.75 (t, J = 8.4 Hz, 1H), 5.29-5.22 (m, 1H), 4.70-4.57 (m, 1H), 4.14-4.08 (m, 1H), 4.02-3.82 (m, 2H), 3.80-3.60 (m, 3H), 3.58-3.31 (m, 3H), 3.17- 3.05 (m, 2H), 3.05-2.95 (m, 1H), 2.94-2.81 (m, 2H), 2.81-2.70 (m, 2H), 2.69-2.55 (m, 2H), 2.54-2.45 (m, 2H), 2.38-2.03 (m, 4H), 2.03-1.67 (m, 6H), 1.66-1.56 (m, 1H), 1.55- 1.44 (m, 7H), 1.43-1.33 (m, 3H), 1.32-1.20 (m, 2H), 1.20-1.08 (m, 2H), 0.88-0.62 (m, 3H). |
| 80 | 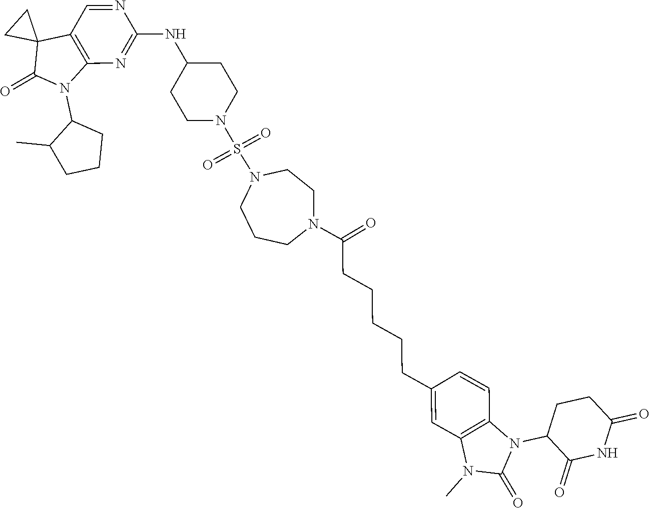 [SMILES: CC1CCCC1N1C(=O)C2(CC2)c2cnc(NC3CCN(S(=O)(=O)N4CCCN(C(=O)CCCCCc5ccc6c(c5)n(C)c(=O)n6C5CCC(=O)NC5=O)CC4)CC3)nc21] | 859.3 | ¹H NMR (400 MHz, DMSO-d₆) δ 11.04 (s, 1H), 7.90-7.59 (m, 1H), 7.19-6.90 (m, 3H), 6.84 (t, J = 7.6 Hz, 1H), 5.33 (dd, J = 12.8, 5.1 Hz, 1H), 4.86-4.08 (m, 1H), 3.78 (s, 1H), 3.61-3.40 (m, 7H), 2.98-2.73 (m, 4H), 2.73-2.50 (m, 5H), 2.37- 2.11 (m, 4H), 2.05-1.80 (m, 6H), 1.79-1.64 (m, 4H), 1.64-1.38 (m, 12H), 1.38-1.10 (m, 4H), 0.88 (d, J = 6.6 Hz, 2H), 0.73 (d, J = 7.0 Hz, 1H). |
| 81 | 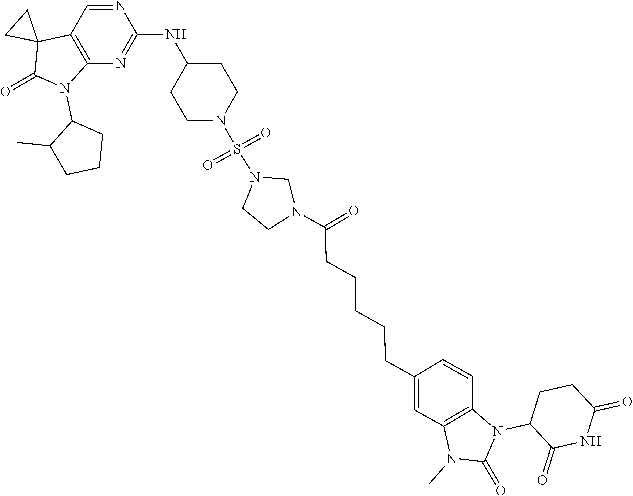 [SMILES: CC1CCCC1N1C(=O)C2(CC2)c2cnc(NC3CCN(S(=O)(=O)N4CCN(C(=O)CCCCCc5ccc6c(c5)n(C)c(=O)n6C5CCC(=O)NC5=O)C4)CC3)nc21] | 830.4 | ¹H NMR (400 MHz, DMSO-d₆) δ 10.98 (s, 1H), 7.69 (d, J = 3.6 Hz, 1H), 7.07-6.70 (m, 4H), 5.31-5.22 (m, 1H), 4.74-4.07 (m, 2H), 4.06- 3.94 (m, 1H), 3.87-3.68 (m, 2H), 3.68-3.50 (m, 4H), 3.49-3.41 (m, 2H), 3.01-2.89 (m, 2H), 2.87-2.76 (m, 1H), 2.69-2.51 (m, 5H), 2.26-2.08 (m, 5H), 2.03-1.78 (m, 6H), 1.69- 1.58 (m, 1H), 1.58-1.42 (m, 8H), 1.42-1.33 (m, 3H), 1.30-1.08 (m, 3H), 0.88-0.74 (m, 3H) |
| 82 | 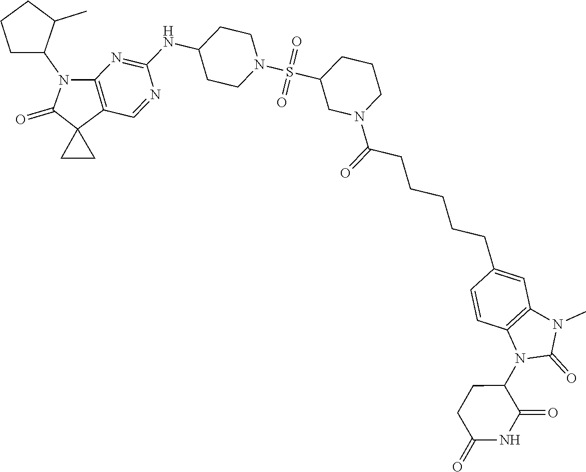 [SMILES: CC1CCCC1N1C(=O)C2(CC2)c2cnc(NC3CCN(S(=O)(=O)C4CCCN(C(=O)CCCCCc5ccc6c(c5)n(C)c(=O)n6C5CCC(=O)NC5=O)C4)CC3)nc21] | 844.5 | ¹H NMR (300 MHz, DMSO-d₆) δ 11.08 (s, 1H), 7.44-7.17 (s, 1H), 7.78 (s, 1H), 7.07-6.97 (m, 2H), 6.85 (d, J = 8.0 Hz, 1H), 5.32 (d, J = 12.3 Hz, 1H), 4.71 (dd, J = 39.5, 10.3 Hz, 1H), 4.19 (s, 1H), 4.04-3.59 (m, 4H), 3.36 (s, 3H), 3.31- 3.10 (m, 2H), 3.10-2.95 (m, 3H), 2.95-2.81 (m, 1H), 2.77-2.55 (m, 5H), 2.26 (d, J = 26.0 Hz, 3H), 2.12-1.85 (m, 6H), 1.85-1.67 (m, 3H), 1.64- 1.41 m, 12H), 1.28 (d, J = 24.7 Hz, 3H), 0.88 (d, J = 6.4 Hz, 2H), 0.74 (d, J = 6.9 Hz, 1H). |
| 83 | 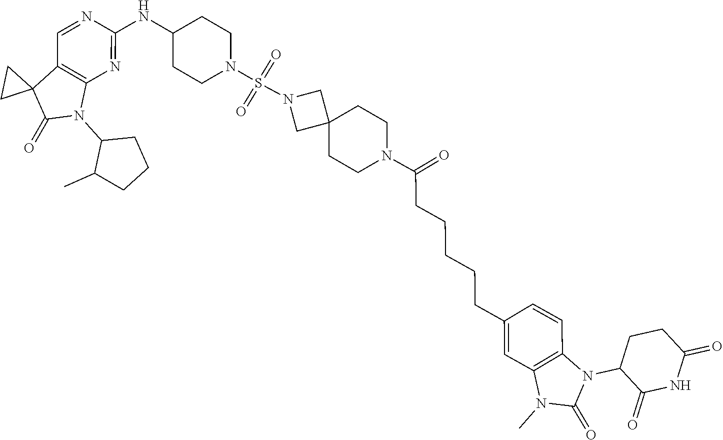 [SMILES: CC1CCCC1N1C(=O)C2(CC2)c2cnc(NC3CCN(S(=O)(=O)N4CC5(CCN(C(=O)CCCCCc6ccc7c(c6)n(C)c(=O)n7C6CCC(=O)NC6=O)CC5)C4)CC3)nc21] | 913.4 | ¹H NMR (300 MHz, DMSO-d₆) δ 11.08 (s, 1H), 7.76 (d, J = 2.6 Hz, 1H), 7.13-6.73 (m, 4H), 5.32 (d, J = 12.0 Hz, 1H), 4.30-3.76 (m, 2H), 3.58 (s, 6H), 3.32 (s, 3H), 3.00-2.82 (m, 3H), 2.76-2.55 (m, 5H), 2.26 (d, J = 7.9 Hz, 3H), 2.05-1.89 (s, 6H), 1.75-1.67 (m, 2H) 1.64-1.54 (m, 7H), 1.53- 1.40 (s, 7H), 1.36-1.21 (m, 9H), 1.21-1.14 (m, 1H), 0.92-0.72 (dd, J = 43.0, 6.9 Hz, 3H). |
| 84 | 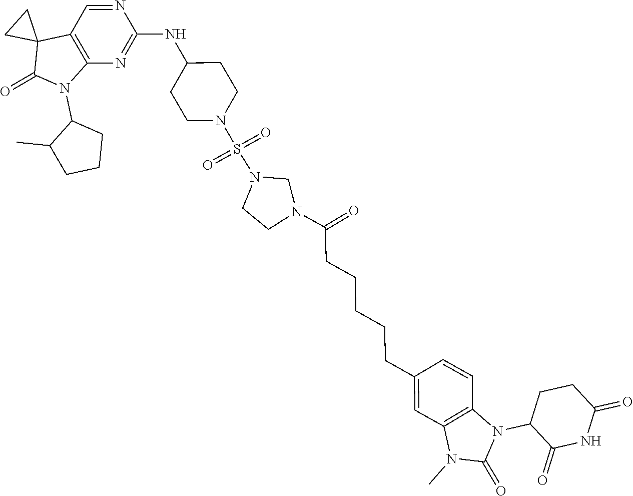 [SMILES: CC1CCCC1N1C(=O)C2(CC2)c2cnc(NC3CCN(S(=O)(=O)N4CCN(C(=O)CCCCCc5ccc6c(c5)n(C)c(=O)n6C5CCC(=O)NC5=O)C4)CC3)nc21] | 858.4 | ¹H NMR (400 MHz, DMSO-d₆) δ 11.07 (s, 1H), 7.76 (d, J = 3.5 Hz, 1H), 7.18-6.92 (m, 3H), 6.88-6.81 (m, 1H), 5.33 (dd, J = 12.7, 5.4 Hz, 1H), 4.80-4.14 (m, 1H), 4.11-3.77 (m, 3H), 3.71- 3.56 (m, 4H), 3.56-3.39 (m, 2H), 3.29-3.23 (m, 1H), 3.08-2.95 (m, 2H), 2.95-2.82 (m, 1H), 2.77- 2.55 (m, 5H), 2.34-2.13 (m, 5H), 2.05-1.88 (m, 4H), 1.87-1.63 (s, 3H), 1.62-1.39 (m, 11H), 1.36-1.18 (s, 7H), 0.88 (d, J = 6.6 Hz, 2H), 0.74 (d, J = 7.0 Hz, 1H). |
| 85 | 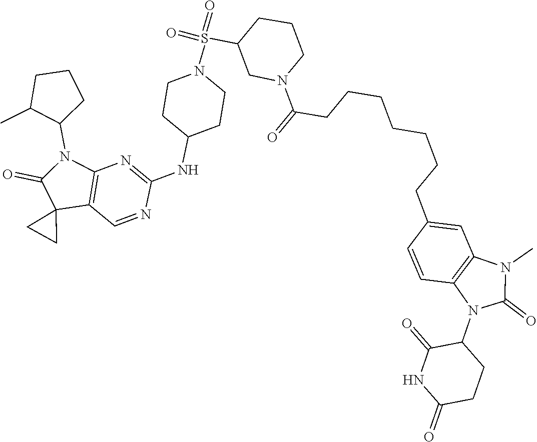 | 872.5 | ¹H NMR (300 MHz, DMSO-d₆) δ 11.07 (s, 1H), 7.76 (d, J = 2.7 Hz, 1H), 7.26-6.93 (m, 3H), 6.90-6.74 (m, 1H), 5.33 (dd, J = 12.8, 5.3 Hz, 1H), 4.88-4.51 (m, 1H), 4.36-4.09 (m, 1H), 4.07-3.73 (m, 2H), 3.72-3.58 (m, 2H), 3.27- 2.81 (m, 5H), 2.80-2.51 (m, 6H), 2.41-2.14 (m, 4H), 2.13-1.62 (m, 11H), 1.62-1.38 (m, 11H), 1.38-1.15 (m, 8H), 0.81 (dd, J = 42.9, 6.7 Hz, 3H). |
| 86 | 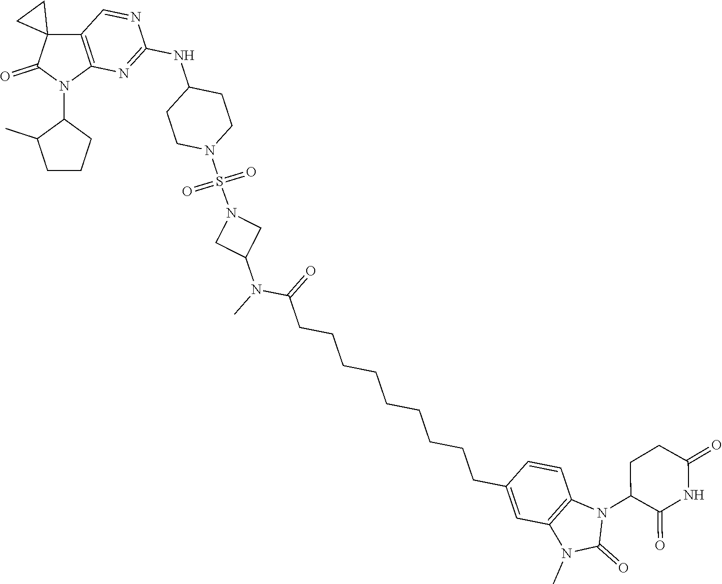 [SMILES: CC1CCCC1N1C(=O)C2(CC2)c2cnc(NC3CCN(S(=O)(=O)N4CC(N(C)C(=O)CCCCCCCCCc5ccc6c(c5)n(C)c(=O)n6C5CCC(=O)NC5=O)C4)CC3)nc21] | 901.5 | ¹H NMR (400 MHz, DMSO-d₆) δ 11.07 (s, 1H), 7.76 (d, J = 3.5 Hz, 1H), 7.20-6.96 (m, 3H), 6.85 (dd, J = 8.1, 1.6 Hz, 1H), 5.33 (dd, J = 12.7, 5.4 Hz, 1H), 5.10-4.69 (m, 1H), 4.18 (m, 1H), 4.07- 3.90 (m, 3H), 3.84 (m, 2H), 3.58 (m, 2H), 3.07- 2.84 (m, 6H), 2.80-2.54 (m, 6H), 2.30 (m, 2H), 2.19 (s, 1H), 2.09-1.86 (m, 5H), 1.86-1.65 (m, 3H), 1.64-1.52 (m, 5H), 1.52-1.38 (m, 6H), 1.27 (m, 11H), 0.88 (d, J-6.6 Hz, 2H), 0.74 (d, J = 7.0 Hz, 1H). |
| 87 | 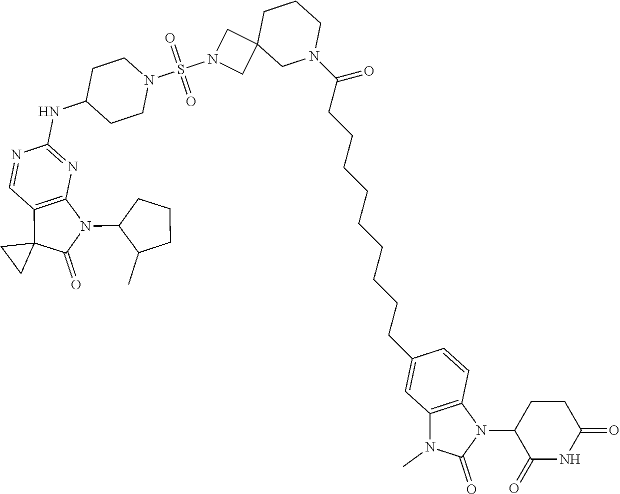 [SMILES: CC1CCCC1N1C(=O)C2(CC2)c2cnc(NC3CCN(S(=O)(=O)N4CC5(CCCN(C(=O)CCCCCCCCCc6ccc7c(c6)n(C)c(=O)n7C6CCC(=O)NC6=O)C5)C4)CC3)nc21] | 941.4 | ¹H NMR (400 MHz, DMSO-d₆) δ 11.08 (s, 1H), 7.76 (d, J = 3.6 Hz, 1H), 7.21-6.90 (m, 3H), 6.89-6.77 (m, 1H), 5.39-5.25 (m, 1H), 4.89- 3.72 (m, 2H), 3.63-3.43 (m, 8H), 3.40-3.35 (m, 2H), 3.31 (s, 3H), 2.99-2.81 (m, 3H), 2.74- 2.52 (m, 5H), 2.39-2.11 (m, 3H), 2.42-1.62 (m, 9H), 1.61-1.34 (m, 12H), 1.30-1.18 (m, 11H), 0.89-0.69 (m, 3H). |
| 88 | 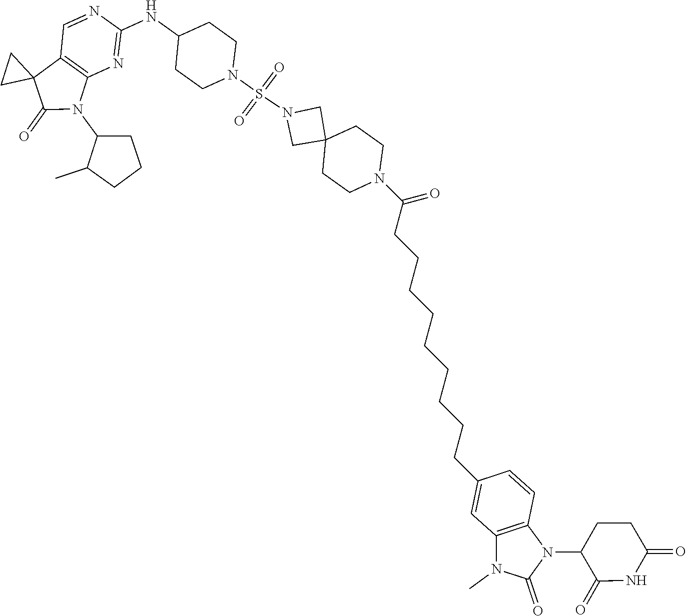 [SMILES: CC1CCCC1N1C(=O)C2(CC2)c2cnc(NC3CCN(S(=O)(=O)N4CC5(CCN(C(=O)CCCCCCCCCc6ccc7c(c6)n(C)c(=O)n7C6CCC(=O)NC6=O)CC5)C4)CC3)nc21] | 941.5 | ¹H NMR (400 MHz, DMSO-d₆) δ 11.08 (s, 1H), 7.76 (d, J = 3.5 Hz, 1H), 7.07 (d, J = 14.7 Hz, 1H), 7.02 (d, J = 1.6 Hz, 1H), 6.99 (d, J = 8.0 Hz, 1H), 6.85 (dd, J = 8.1, 1.5 Hz, 1H), 5.33 (dd, J = 12.8, 5.4 Hz, 1H), 4.87-4.09 (m, 1H), 3.82 (s, 1H), 3.58 (m, 3H), 3.38 (d, J = 9.1 Hz, 4H), 3.32 (s, 2H), 2.90 (m, 3H), 2.76-2.56 (m, 5H), 2.31- 2.14 (m, 3H), 2.05-1.74 (m, 6H), 1.68 (s, 3H), 1.65-1.54 (m, 6H), 1.54-1.38 (q, J = 5.6, 4.6 Hz, 7H), 1.33-1.19 (m, 11H), 0.88 (d, J = 6.6 Hz, 2H), 0.74 (d, J = 7.0 Hz, 1H). |
| 89 | 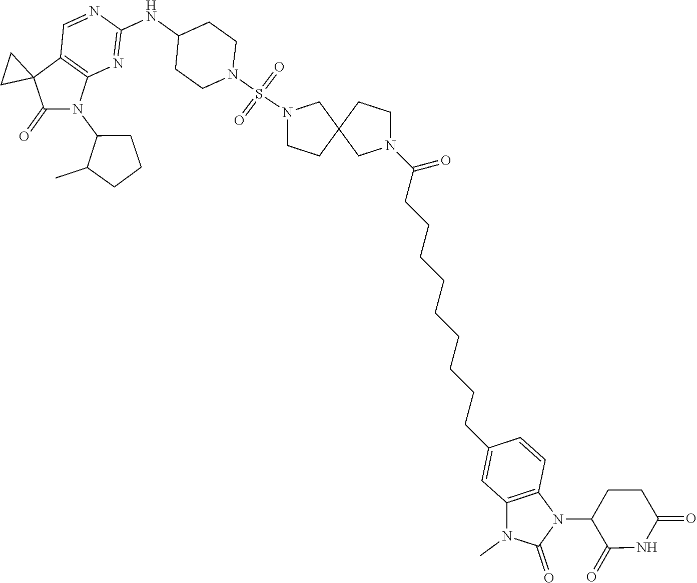 [SMILES: CC1CCCC1N1C(=O)C2(CC2)c2cnc(NC3CCN(S(=O)(=O)N4CCC5(CCN(C(=O)CCCCCCCCCc6ccc7c(c6)n(C)c(=O)n7C6CCC(=O)NC6=O)C5)C4)CC3)nc21] | 941.3 | ¹H NMR (400 MHz, DMSO-d₆) δ 11.07 (s, 1H), 7.76 (d, J = 3.6, 1H), 7.15-6.97 (m, 3H), 6.89- 6.79 (m, 1H), 5.39-5.23 (m, 1H), 4.90-3.74 (m, 2H), 3.61-3.33 (m, 8H), 3.25-3.11 (m, 3H), 2.96- 2.82 (m, 3H), 2.77-2.54 (m, 5H), 2.26-2.11 (m, 3H), 2.05-1.62 (m, 12H), 1.69-1.31 (m, 11H), 1.33-1.18 (m, 11H), 0.94-0.68 (m, 3H). |
| 90 | 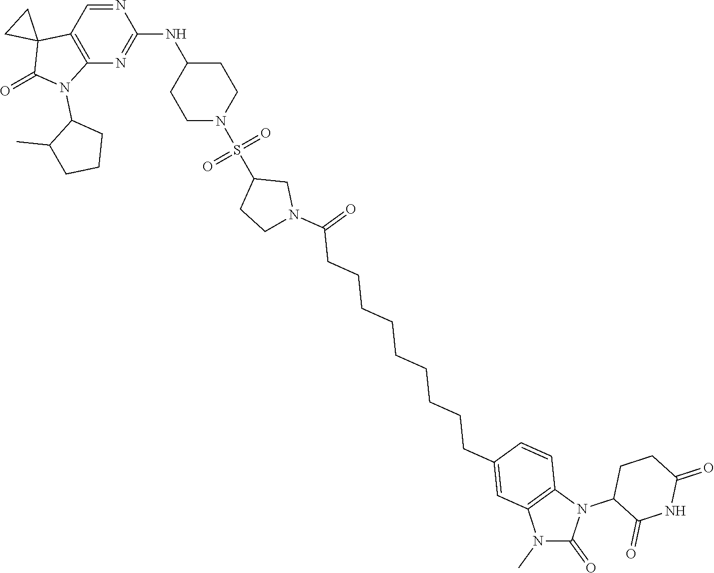 [SMILES: CC1CCCC1N1C(=O)C2(CC2)c2cnc(NC3CCN(S(=O)(=O)C4CCN(C(=O)CCCCCCCCCc5ccc6c(c5)n(C)c(=O)n6C5CCC(=O)NC5=O)C4)CC3)nc21] | 886.5 | ¹H NMR (400 MHz, DMSO-d₆) δ 11.06 (s, 1H), 7.76 (d, J = 3.6 Hz, 1H), 7.18-6.94 (m, 3H), 6.90-6.77 (m, 1H), 5.40-5.26 (m, 1H), 4.83-4.12 (m, 1H), 4.10-3.92 (m, 1H), 3.90-3.79 (m, 2H), 3.70-3.60 (m, 6H), 3.07-2.83 (m, 3H), 2.77-2.56 (m, 5H), 2.36-2.12 (m, 5H), 2.11-1.62 (m, 8H), 1.61-1.38 (m, 11H), 1.34-1.1 (m, 11H),0.91-0.69 (m, 3H). |
| 91 | 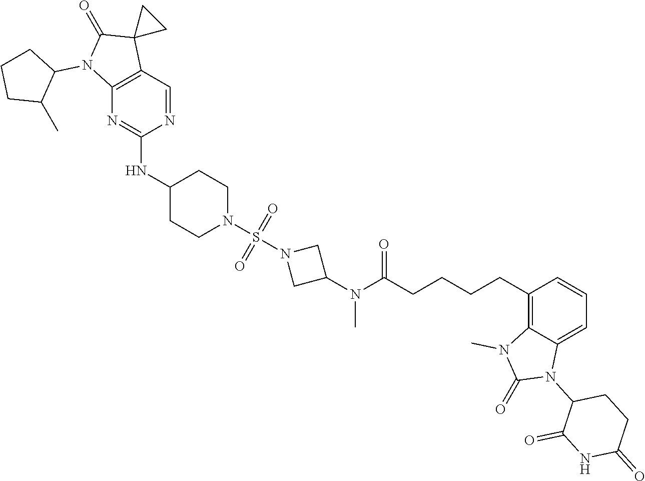 [SMILES: CC1CCCC1N1C(=O)C2(CC2)c2cnc(NC3CCN(S(=O)(=O)N4CC(N(C)C(=O)CCCCc5cccc6c5n(C)c(=O)n6C5CCC(=O)NC5=O)C4)CC3)nc21] | 831.3 | ¹H NMR (400 MHz, DMSO-d₆) δ 11.09 (s, 1H), 7.76 (d, J = 3.6 Hz, 1H), 7.18-6.76 (m, 4H), 5.43-5.29 (m, 1H), 5.13-4.66 (m, 1H), 4.24- 3.77 (m, 6H), 3.65-3.53 (m, 5H), 3.05-2.82 (m, 8H), 2.79-2.61 (m, 3H), 2.42-1.70 (m, 9H), 1.66-1.18 (m, 12H), 0.93-0.68 (m, 3H). |
| 92 | 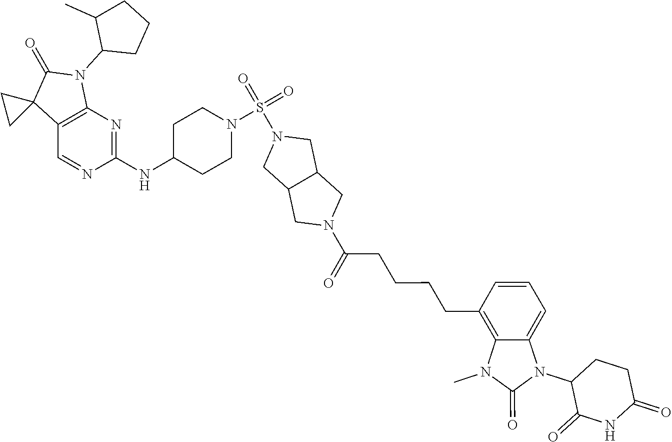 [SMILES: CC1CCCC1N1C(=O)C2(CC2)c2cnc(NC3CCN(S(=O)(=O)N4CC5CN(C(=O)CCCCc6cccc7c6n(C)c(=O)n7C6CCC(=O)NC6=O)CC5C4)CC3)nc21] | 857.4 | 1H NMR (400 MHz, DMSO-d6) § 11.01(s, 1H), 7.68 (d, J = 3.4 Hz, 1H), 7.04-6.74 (m, 4H), 5.33-5.23 (m, 1H), 4.74-4.08 (m, 1H), 3.87- 3.66 (m, 1H), 3.63-3.41 (m, 7H), 3.40-3.31 (m, 2H), 3.21-3.10 (m, 2H), 3.08-2.96 (m, 2H), 2.96-2.80 (m, 7H), 2.76-2.52 (m, 3H), 2.25- 2.18 (m, 2H), 2.17-2.05(m, 1H), 1.97-1.67 (m, 6H), 1.66-1.44 (m, 7H), 1.44-1.29 (m, 4H), 1.23-1.07 (m, 1H), 0.86-0.61 (m, 3H). |
| 93 | 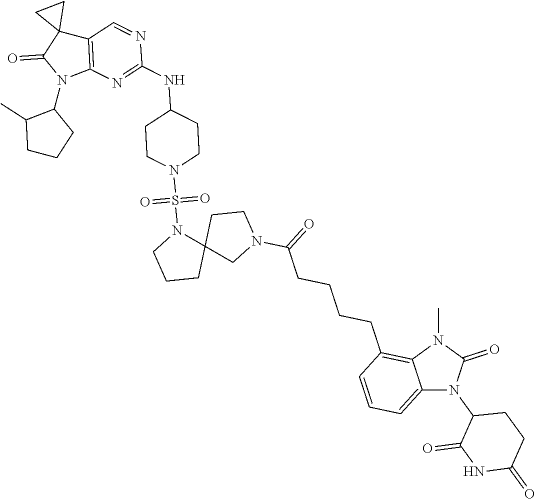 [SMILES: CC1CCCC1N1C(=O)C2(CC2)c2cnc(NC3CCN(S(=O)(=O)N4CCCC45CCN(C(=O)CCCCc4cccc6c4n(C)c(=O)n6C4CCC(=O)NC4=O)C5)CC3)nc21] | 857.4 | ¹H NMR (400 MHz, DMSO-d₆) 8 11.08 (s, 1H), 7.76 (dd, J = 3.7, 1.2 Hz, 1H), 7.07 (s, 1H), 6.99- 6.90 (m, 2H), 6.87 (dd, J = 5.9, 3.2 Hz, 1H), 5.36 (dd, J = 12.5, 5.4 Hz, 1H), 4.78-4.17 (dd, J = 234.7, 9.1 Hz, 1H), 3.80 (s, 1H), 3.74-3.58 (m, 2H), 3.52 (s, 5H), 3.43-3.35 (m, 2H), 3.19- 3.06 (m, 1H), 2.97-2.79 (m, 5H), 2.77-2.54 (m, 4H), 2.54-2.52 (m, 1H), 2.37-2.11 (m, 3H), 2.04-1.72 (m, 12H), 1.70-1.47 (m, 8H), 1.47- 1.41 (m, 2H), 0.88 (dd, J = 6.6, 2.5 Hz, 2H), 0.74 (dd, J = 7.0, 2.3 Hz, 1H). |
| 94 | 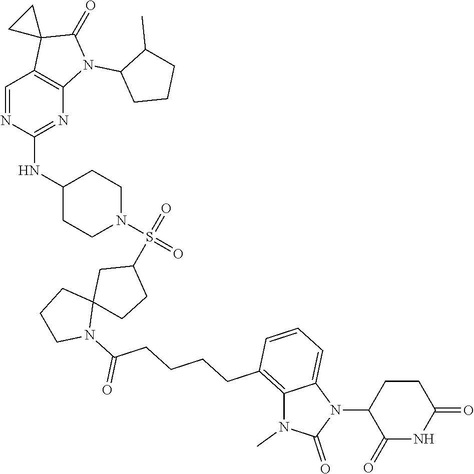 [SMILES: CC1CCCC1N1C(=O)C2(CC2)c2cnc(NC3CCN(S(=O)(=O)C4CCC5(CCCN5C(=O)CCCCc5cccc6c5n(C)c(=O)n6C5CCC(=O)NC5=O)C4)CC3)nc21] | 871.5 | ¹H NMR (400 MHz, DMSO-d₆) 8 11.02 (s, 1H), 7.69 (d, J = 3.5 Hz, 1H), 7.16-6.50 (m, 4H), 5.29 (dd, J = 12.5, 5.3 Hz, 1H), 4.81-4.01 (m, 1H), 3.87-3.62 (m, 2H), 3.57-3.45 (m, 4H), 3.44-3.32 (m, 3H), 3.17 (q, J = 8.1 Hz, 2H), 2.95-2.69 (m, 7H), 2.69-2.49 (m, 31H), 2.23 (s, 2H), 2.19-2.05 (m, 1H), 1.98-1.67 (m, 10H), 1.65-1.26 (m, 12H), 1.23-1.08 (m, 1H), 0.88-0.52 (m, 3H). |
| 95 | 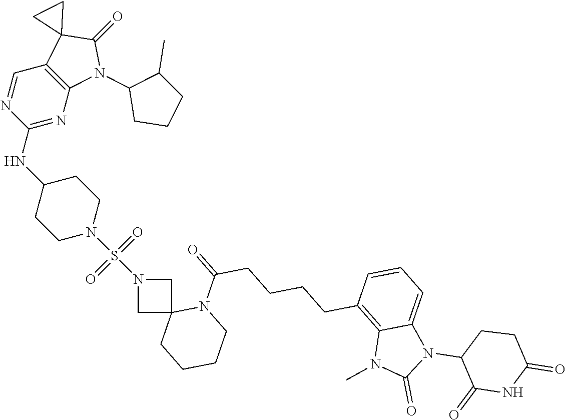 [SMILES: CC1CCCC1N1C(=O)C2(CC2)c2cnc(NC3CCN(S(=O)(=O)N4CC5(CCCCN5C(=O)CCCCc5cccc6c5n(C)c(=O)n6C5CCC(=O)NC5=O)C4)CC3)nc21] | 871.5 | ¹H NMR (400 MHz, DMSO-d₆) 8 11.08 (s, 1H), 7.76 (d, J = 3.5 Hz, 1H), 7.09 (s, 1H), 7.01- 6.92 (m, 2H), 6.90-6.79 (m, 1H), 5.36 (dd, J = 12.4, 5.4 Hz, 1H), 4.48 (dd, J = 234.9, 8.9 Hz, 1H), 3.82 (d, J = 8.2 Hz, 3H), 3.67 (d, J = 8.0 Hz, 2H), 3.61-3.48 (m, 5H), 3.24 (s, 2H), 3.00- 2.81 (m, 5H), 2.76-2.56 (m, 3H), 2.43-2.28 (m, 2H), 2.28-2.11 (m, 1H), 2.04-1.74 (m, 8H), 1.74-1.54 (m, 9H), 1.53-1.40 (m, 4H), 1.35 (s, 2H), 1.23 (t, J = 10.0 Hz, 1H), 0.81 (dd, J = 57.2, 6.7 Hz, 3H). |
| 96 | 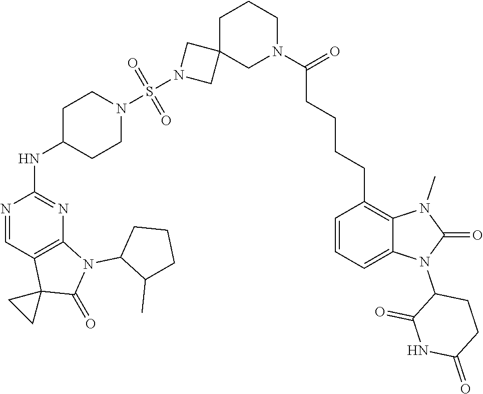 [SMILES: CC1CCCC1N1C(=O)C2(CC2)c2cnc(NC3CCN(S(=O)(=O)N4CC5(CCCN(C(=O)CCCCc6cccc7c6n(C)c(=O)n7C6CCC(=O)NC6=O)C5)C4)CC3)nc21] | 871.4 | ¹H NMR (400 MHz, DMSO-d₆) δ 11.08 (s, 1H), 7.76 (d, J = 3.9 Hz, 1H), 7.07 (s, 1H), 6.95 (d, J = 5.7 Hz, 2H), 6.87-6.77 (m, 1H), 5.35 (dd, J = 12.6, 5.5 Hz, 1H), 4.82-4.10 (m, 1H), 3.81 (s, 1H), 3.64-3.49 (m, 8H), 3.46 (s, 3H), 3.30-3.27 (m, 1H), 2.97-2.82 (m, 5H), 2.76-2.57 (m, 3H), 2.44 (s, 1H), 2.35 (m, 1H), 2.20 (s, 1H), 2.03- 1.78 (m, 6H), 1.77-1.69 (m, 3H), 1.69-1.54 (m, 6H), 1.53-1.35 (m, 7H), 1.23 (s, 1H), 0.88 (d, J = 6.4 Hz, 2H), 0.73 (d, J = 6.9 Hz, 1H). |
| 97 | 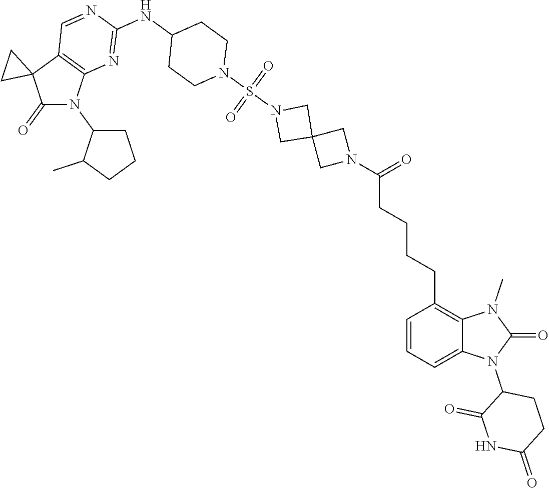 [SMILES: CC1CCCC1N1C(=O)C2(CC2)c2cnc(NC3CCN(S(=O)(=O)N4CC5(CN(C(=O)CCCCc6cccc7c6n(C)c(=O)n7C6CCC(=O)NC6=O)C5)C4)CC3)nc21] | 843.5 | 1H NMR (400 MHz, DMSO-d6) δ 11.08 (s, 1H), 7.76 (d, J = 3.5 Hz, 1H), 7.07 (s, 1H), 6.99- 6.89 (m, 2H), 6.85 (dd, J = 5.8, 3.1 Hz, 1H), 5.36 (dd, J = 12.6, 5.4 Hz, 1H), 4.80-4.10 (m, 3H), 3.96 (d, J = 17.8 Hz, 6H), 3.84-3.74 (m, 1H), 3.55 (s, 5H), 2.91 (d, J = 18.5 Hz, 5H), 2.77- 2.57 (m, 3H), 2.27 (d, J = 50.1 Hz, 1H), 2.06 (d, J = 6.2 Hz, 1H), 2.01-1.77 (m, 6H), 1.68 (s, 1H), 1.62-1.30 (m, 11H), 1.24 (t, J = 10.0 Hz, 1H), 0.81 (dd, J = 57.2, 6.8 Hz, 3H). |
| 98 | 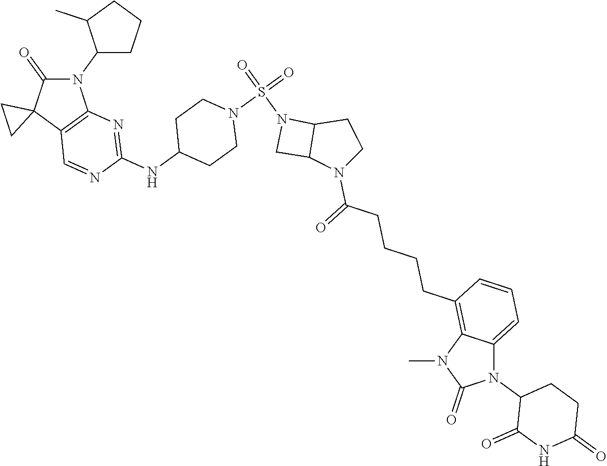 [SMILES: CC1CCCC1N1C(=O)C2(CC2)c2cnc(NC3CCN(S(=O)(=O)N4CC5C4CCN5C(=O)CCCCc4cccc5c4n(C)c(=O)n5C4CCC(=O)NC4=O)CC3)nc21] | 843.3 | ¹H NMR (400 MHz, DMSO-d₆) δ 11.10 (s, 1H), 7.76 (d, J = 3.5 Hz, 1H), 7.09 (s, 1H), 6.95 (dd, J = 8.3, 5.3 Hz, 2H), 6.86 (dt, J = 6.2, 3.1 Hz, 1H), 5.36 (dd, J = 12.6, 5.4 Hz, 1H), 4.85 (dt, J = 41.0, 5.3 Hz, 1H), 4.51 (ddd, J = 32.6, 8.6, 3.8 Hz, 1H), 4.24-3.89 (m, 3H), 3.89-3.66 (m, 2H), 3.63-3.40 (m, 6H), 3.29 (d, J = 8.2 Hz, 1H), 2.88 (dd, J = 21.5, 8.4 Hz, 5H), 2.76-2.55 (m, 3H), 2.47-2.27 (m, 1H), 2.27-2.09 (m, 1H), 2.05-1.80 (m, 7H), 1.80-1.52 (m, 8H), 1.52-1.40 (m, 4H), 0.87 (d, J = 6.6 Hz, 2H), 0.73 (d, J = 6.9 Hz, 1H). |
| 99 | 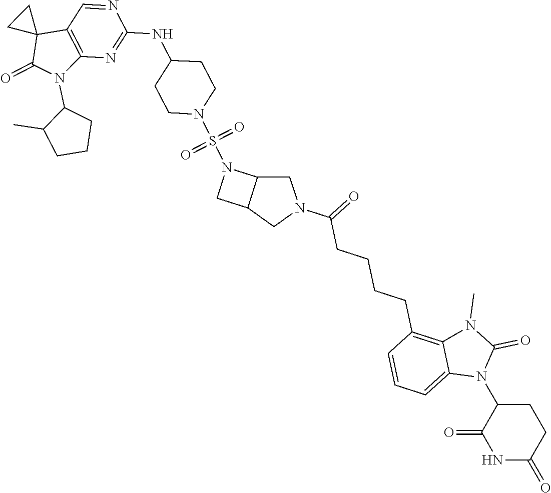 [SMILES: CC1CCCC1N1C(=O)C2(CC2)c2cnc(NC3CCN(S(=O)(=O)N4CC5CN(C(=O)CCCCc6cccc7c6n(C)c(=O)n7C6CCC(=O)NC6=O)CC54)CC3)nc21] | 843.5 | ¹H NMR (400 MHz, DMSO-d₆) δ 11.08 (s, 1H), 7.82-7.68 (m, 1H), 7.06 (s, 1H), 6.99-6.75 (m, 3H), 5.35 (dd, J = 12.5, 5.3 Hz, 1H), 4.73 (dd, J = 7.1, 3.8 Hz, 1H), 4.17 (d, J = 9.2 Hz, 1H), 3.97 (dd, J = 23.4, 11.9 Hz, 2H), 3.88-3.63 (m, 2H), 3.60-3.46 (m, 5H), 3.46-3.39 (m, 2H), 3.23-3.03 (m, 2H), 3.01-2.56 (m, 8H), 2.47-2.27 (m, 2H), 2.19 (s, 1H), 2.03-1.74 (m, 6H), 1.65 (s, 5H), 1.59-1.38 (m, 6H), 1.22 (d, J = 15.7 Hz, 1H), 0.87 (dd, J = 6.6, 3.8 Hz, 2H), 0.72 (dd, J = 7.0, 3.9 Hz, 1H). |
| 100 | 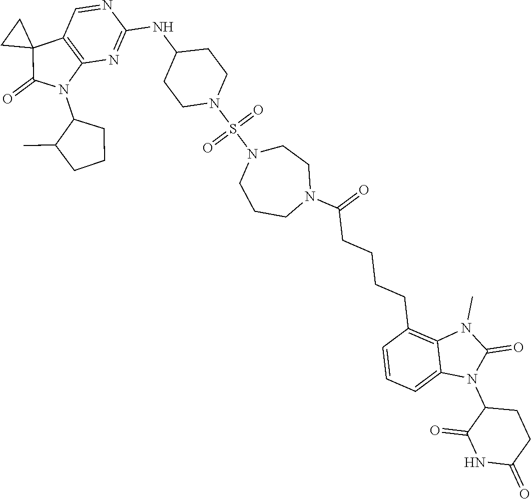 [SMILES: CC1CCCC1N1C(=O)C2(CC2)c2cnc(NC3CCN(S(=O)(=O)N4CCCN(C(=O)CCCCc5cccc6c5n(C)c(=O)n6C5CCC(=O)NC5=O)CC4)CC3)nc21] | 845.4 | ¹H NMR (400 MHz, DMSO-d₆) δ 11.07 (s, 1H), 7.75 (t, J = 3.6 Hz, 1H), 7.24-6.79 (m, 4H), 5.40-5.32 (m, 1H), 4.82-4.12 (m, 1H), 3.78 (s, 1H), 3.60-3.35 (m, 12H), 2.96-2.55 (m, 8H), 2.38-2.11 (m, 3H), 2.03-1.16 (m, 21H), 0.92- 0.69 (m, 3H). |
| 101 | 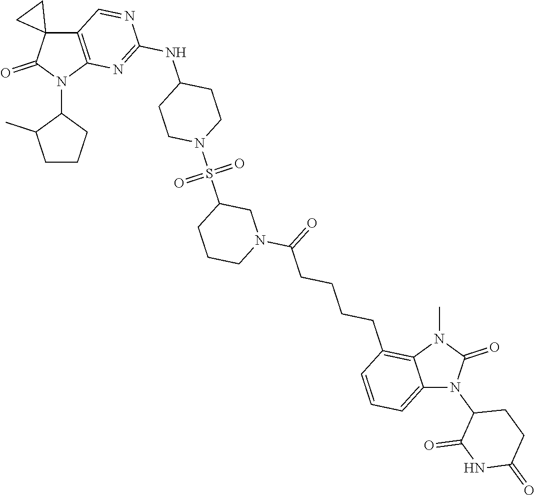 [SMILES: CC1CCCC1N1C(=O)C2(CC2)c2cnc(NC3CCN(S(=O)(=O)C4CCCN(C(=O)CCCCc5cccc6c5n(C)c(=O)n6C5CCC(=O)NC5=O)C4)CC3)nc21] | 830.3 | ¹H NMR (400 MHz, DMSO-d₆) δ 11.07 (s, 1H), 7.76 (d, J = 3.9 Hz, 1H), 7.17-7.02 (m, 1H), 7.00-6.82 (m, 3H), 5.36 (dd, J = 12.6, 5.4 Hz, 1H), 4.85-4.56 (m, 1H), 4.30-3.95 (m, 2H), 3.92-3.76 (m, 1H), 3.74-3.60 (m, 2H), 3.56 (s, 3H), 3.24-2.77 (m, 7H), 2.78-2.56 (m, 4H), 2.46-2.29 (m, 2H), 2.29-2.13 (m, 1H), 2.13-2.03 (m, 1H), 2.03-1.38 (m, 20H), 1.30- 1.14 (m, 1H), 0.81 (dd, J = 56.8, 6.7 Hz, 3H). |
| 102 | 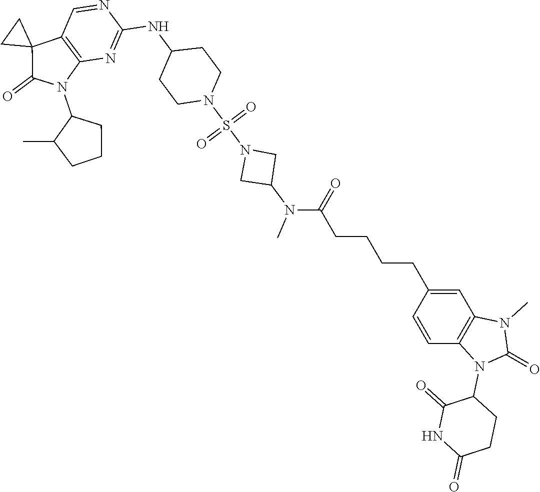 [SMILES: CC1CCCC1N1C(=O)C2(CC2)c2cnc(NC3CCN(S(=O)(=O)N4CC(N(C)C(=O)CCCCc5ccc6c(c5)n(C)c(=O)n6C5CCC(=O)NC5=O)C4)CC3)nc21] | 931.5 | 1H NMR (400 MHz, DMSO-d6) δ 11.07 (s, 1H), 7.76 (d, J = 3.5 Hz, 1H), 7.20-6.94 (m, 3H), 6.86 (dd, J = 8.1, 1.5 Hz, 1H), 5.33 (dd, J = 12.7, 5.4 Hz, 1H), 5.08-4.69 (m, 1H), 4.18 (d, J = 9.2 Hz, 1H), 4.07-3.73 (m, 5H), 3.58 (d, J = 11.9 Hz, 2H), 3.34 (s, 1H), 3.05-2.83 (m, 6H), 2.78-2.56 (m, 5H), 2.33 (dd, J = 9.1, 4.8 Hz, 2H), 2.18 (dt, J = 24.7, 9.3 Hz, 2H), 2.07-1.73 (m, 7H), 1.72-1.39 (m, 11H), 1.24 (t, J = 8.5 Hz, 1H), 0.88 (d, J = 6.6 Hz, 2H), 0.74 (d, J = 7.0 Hz, 1H). |
| 103 | 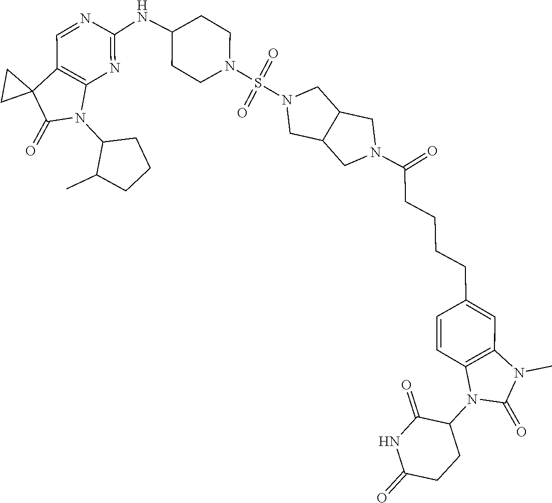 [SMILES: CC1CCCC1N1C(=O)C2(CC2)c2cnc(NC3CCN(S(=O)(=O)N4CC5CN(C(=O)CCCCc6ccc7c(c6)n(C)c(=O)n7C6CCC(=O)NC6=O)CC5C4)CC3)nc21] | 857.3 | 1H NMR (400 MHz, DMSO-d6) 8 11.08 (s, 1H), 7.75 (d, J = 3.5 Hz, 1H), 7.15-6.75 (m, 4H), 5.33 (dd, J = 12.7, 5.4 Hz, 1H), 4.83-4.10 (m, 1H), 3.80 (s, 1H), 3.67-3.35 (m, 7H), 3.22 (dd, J = 12.2, 4.8 Hz, 2H), 3.09 (td, J = 10.3, 4.5 Hz, 2H), 3.02-2.80 (m, 5H), 2.78-2.54 (m, 5H), 2.32-2.11 (m, 3H), 2.06-1.73 (m, 7H), 1.73- 1.36 (m, 11H), 1.28-1.17 (m, 2H), 0.91-0.70 (m, 3H). |
| 104 | 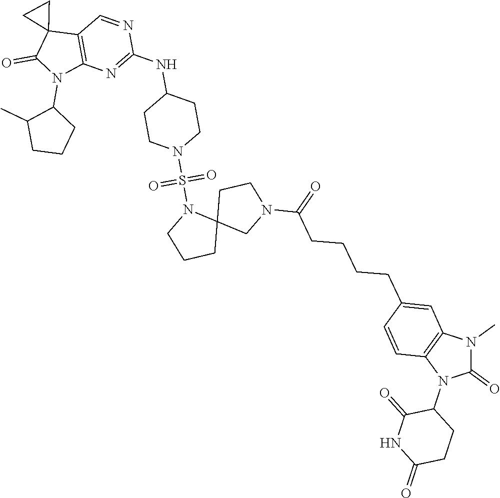 [SMILES: CC1CCCC1N1C(=O)C2(CC2)c2cnc(NC3CCN(S(=O)(=O)N4CCCC45CCN(C(=O)CCCCc4ccc6c(c4)n(C)c(=O)n6C4CCC(=O)NC4=O)C5)CC3)nc21] | 871.4 | ¹H NMR (300 MHz, DMSO-d₆) δ 11.07 (s, 1H), 7.76 (d, J = 2.8 Hz, 1H), 7.20-6.96 (m, 3H), 6.96-6.84 (m, 1H), 5.33 (dd, J = 12.7, 5.4 Hz, 1H), 4.87-4.04 (m, 1H), 3.80 (s, 1H), 3.75- 3.48 (m, 5H), 3.44-3.34 (m, 1H), 3.24-3.06 (m, 1H), 2.99-2.79 (m, 3H), 2.78-2.56 (m, 6H), 2.54 (s, 1H), 2.34-2.11 (m, 3H), 2.09-1.69 (m, 13H), 1.68-1.38 (m, 12H), 1.22 (d, J = 7.8 Hz, 1H), 0.88 (d, J = 6.6 Hz, 2H), 0.74 (d, J = 6.9 Hz, 1H). |
| 105 | 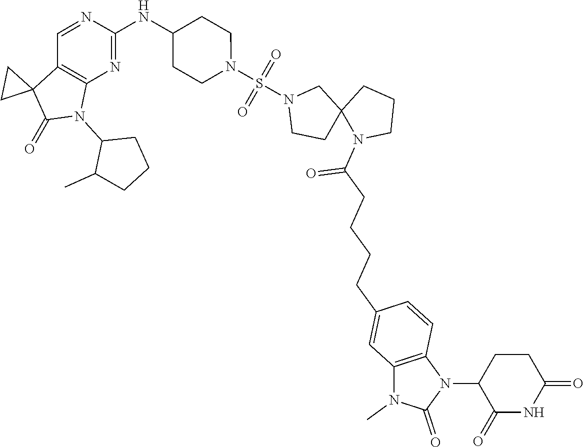 [SMILES: CC1CCCC1N1C(=O)C2(CC2)c2cnc(NC3CCN(S(=O)(=O)N4CCC5(CCCN5C(=O)CCCCc5ccc6c(c5)n(C)c(=O)n6C5CCC(=O)NC5=O)C4)CC3)nc21] | 871.3 | ¹H NMR (400 MHz, DMSO-d₆) δ 11.07 (s, 1H), 7.76 (d, J = 3.6 Hz, 1H), 7.08-6.79 (m, 4H), 5.38-5.27 (m, 1H), 4.84-4.10 (m, 1H), 3.92- 3.40 (m, 7H), 3.28-3.16 (m, 5H), 2.97-2.55 (m, 9H), 2.32-2.13 (m, 3H), 2.03-1.77 (m, 10H), 1.72-1.12 (m, 13H), 0.92-0.68 (m, 3H). |
| 106 | 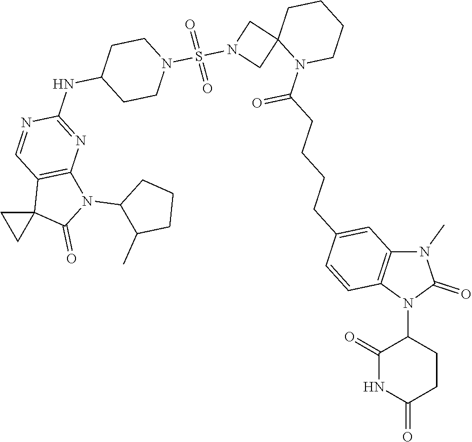 [SMILES: CC1CCCC1N1C(=O)C2(CC2)c2cnc(NC3CCN(S(=O)(=O)N4CC5(CCCCN5C(=O)CCCCc5ccc6c(c5)n(C)c(=O)n6C5CCC(=O)NC5=O)C4)CC3)nc21] | 871.3 | ¹H NMR (400 MHz, DMSO-d₆) δ 11.07 (s, 1H), 7.76 (d, J = 3.5 Hz, 1H), 7.14-6.94 (m, 3H), 6.86 (d, J = 7.7 Hz, 1H), 5.33 (dd, J = 12.7, 5.4 Hz, 1H), 4.18 (d, J = 9.1 Hz, 1H), 3.81 (d, J = 8.2 Hz, 3H), 3.66 (d, J = 7.9 Hz, 3H), 3.56 (d, J = 12.0 Hz, 3H), 3.23 (s, 3H), 2.90 (q, J = 12.7 Hz, 3H), 2.77-2.56 (m, 5H), 2.31 (t, J = 7.2 Hz, 2H), 2.20 (s, 2H), 1.89 (dd, J = 45.0, 31.4 Hz, 8H), 1.74-1.40 (m, 14H), 1.38-1.17 (m, 4H), 0.88 (d, J = 6.6 Hz, 2H), 0.73 (d, J = 7.0 Hz, 1H). |
| 107 | 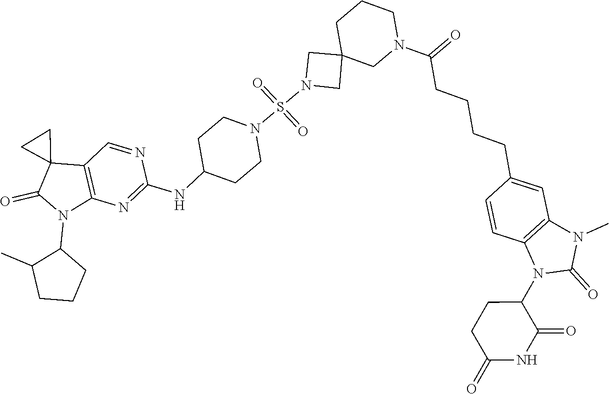 | 871.3 | ¹H NMR (400 MHz, DMSO-d₆) δ 11.06 (s, 1H), 7.76 (d, J = 3.6 Hz, 1H), 7.15-6.73 (m, 4H), 5.41-5.21 (m, 1H), 4.81-3.74 (m, 2H), 3.66-3.41 (m, 9H), 3.27-3.14 (m, 2H), 2.97-2.54 (m, 8H), 2.43-2.12 (m, 3H), 2.05-2.16 (m, 24H), 0.92- 0.69 (m, 3H). |
| 108 | 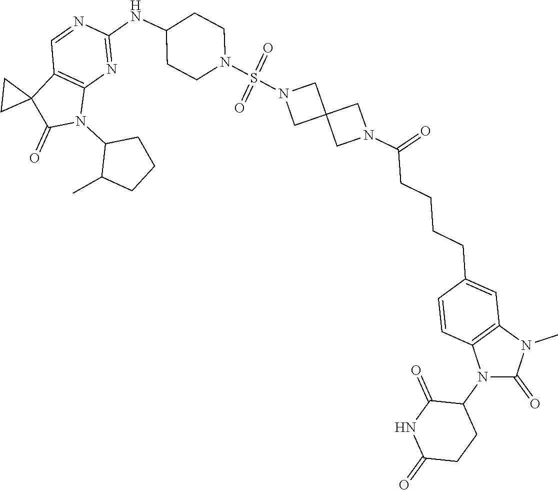 [SMILES: CC1CCCC1N1C(=O)C2(CC2)c2cnc(NC3CCN(S(=O)(=O)N4CC5(CN(C(=O)CCCCc6ccc7c(c6)n(C)c(=O)n7C6CCC(=O)NC6=O)C5)C4)CC3)nc21] | 843.3 | ¹H NMR (300 MHz, DMSO-d₆) δ 11.07 (s, 1H), 7.76 (d, J = 2.7 Hz, 1H), 7.25-6.94 (m, 3H), 6.94-6.80 (m, 1H), 5.34 (dd, J = 12.7, 5.3 Hz, 1H), 4.89-4.08 (m, 3H), 4.10-3.72 (m, 7H), 3.56 (d, J = 11.9 Hz, 2H), 3.24-2.81 (m, 3H), 2.78-2.53 (m, 5H), 2.36-2.16 (m, 2H), 2.12- 1.65 (m, 10H), 1.66-1.35 (m, 10H), 1.33-1.03 (m, 2H), 0.81 (dd, J = 42.9, 6.8 Hz, 3H). |
| 109 | 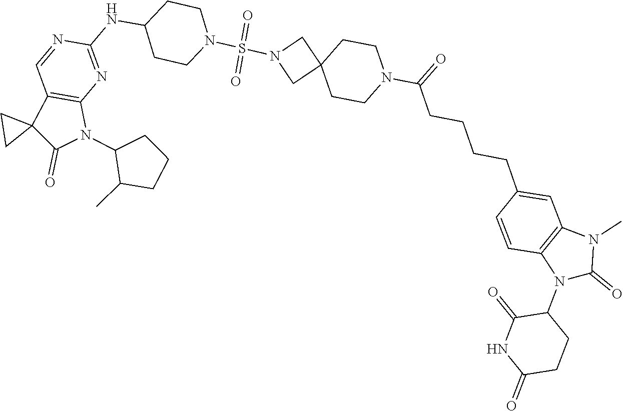 [SMILES: CC1CCCC1N1C(=O)C2(CC2)c2cnc(NC3CCN(S(=O)(=O)N4CC5(CCN(C(=O)CCCCc6ccc7c(c6)n(C)c(=O)n7C6CCC(=O)NC6=O)CC5)C4)CC3)nc21] | 871.3 | ¹H NMR (400 MHz, DMSO-d₆) δ 11.08 (s, 1H), 7.77 (s, 1H), 7.20-6.96 (m, 3H), 6.89-6.84 (m, 1H), 5.39-5.29 (m, 1H), 4.83-3.70 (m, 2H), 3.64-3.52 (s, 6H), 3.41-3.36 (m, 3H), 3.31 (s, 3H), 2.97-2.84 (m, 3H), 2.77-2.57 (m, 5H), 2.35-2.14 (m, 3H), 2.05-1.15 (m, 23H), 0.91- 0.69 (m, 3H). |
| 110 | 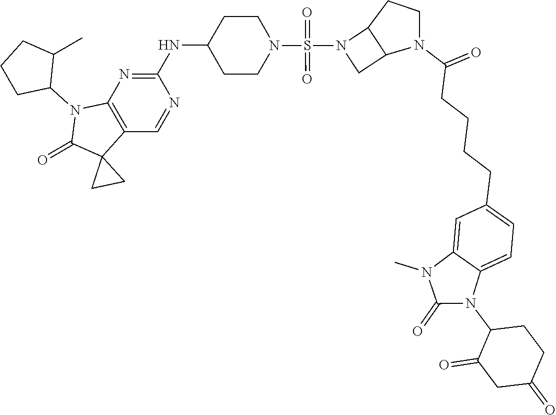 [SMILES: CC1CCCC1N1C(=O)C2(CC2)c2cnc(NC3CCN(S(=O)(=O)N4CC5C4CCN5C(=O)CCCCc4ccc5c(c4)n(C)c(=O)n5C4CCC(=O)CC4=O)CC3)nc21] | 843.4 | ¹H NMR (400 MHz, DMSO-d₆) δ 11.09 (s, 1H), 7.76 (d, J = 3.5 Hz, 1H), 7.19-6.95 (m, 3H), 6.94-6.77 (m, 1H), 5.34 (dd, J = 12.7, 5.4 Hz, 1H), 4.95-4.69 (m, 1H), 4.61-4.38 (m, 1H), 4.26-4.07 (m, 1H), 4.07-3.87 (m, 2H), 3.87- 3.67 (m, 2H), 3.61-3.40 (m, 3H), 3.35-3.20 (m, 2H), 2.99-2.78 (m, 3H), 2.76-2.57 (m, 5H), 2.45-2.09 (m, 3H), 2.04-1.75 (m, 9H), 1.74-1.37 (m, 11H), 1.32-1.15 (m, 1H), 0.96- 0.39 (m, 3H). |
| 111 | 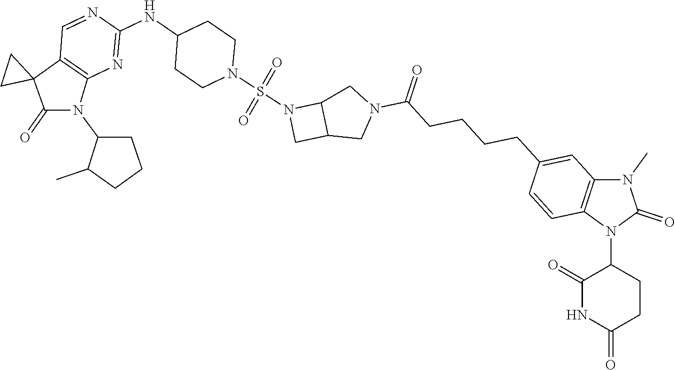 [SMILES: CC1CCCC1N1C(=O)C2(CC2)c2cnc(NC3CCN(S(=O)(=O)N4CC5CN(C(=O)CCCCc6ccc7c(c6)n(C)c(=O)n7C6CCC(=O)NC6=O)CC54)CC3)nc21] | 843.5 | ¹H NMR (400 MHz, DMSO-d₆) δ 10.99 (s, 1H), 7.71-7.65 (m, 1H), 7.15-6.65 (m, 4H), 5.30- 5.13 (m, 1H), 4.77-4.57 (m, 1H), 4.16-3.55 (m, 5H), 3.51-3.32 (m, 4H), 3.25-2.49 (m, 13H), 2.32-2.07 (m, 3H), 1.98-1.67 (m, 7H), 1.63-1.07 (m, 11H), 0.83-0.59 (m, 3H). |
| 112 | 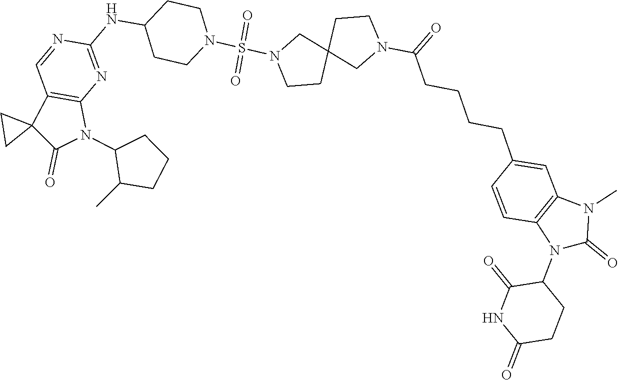 [SMILES: CC1CCCC1N1C(=O)C2(CC2)c2cnc(NC3CCN(S(=O)(=O)N4CCC5(CCN(C(=O)CCCCc6ccc7c(c6)n(C)c(=O)n7C6CCC(=O)NC6=O)C5)C4)CC3)nc21] | 871.4 | ¹H NMR (400 MHz, DMSO-d₆) δ 11.07 (s, 1H), 7.76 (d, J = 3.6 Hz, 1H), 7.18-6.74 (m, 4H), 5.39-5.22 (m, 1H), 4.89-3.69 (m, 2H), 3.64-3.39 (m, 4H), 3.25-3.11 (m, 3H), 2.96-2.56 (m, 8H), 2.29-2.14 (m, 3H), 2.04-1.74 (m, 14H), 1.66- 1.16 (m, 14H), 0.91-0.69 (m, 3H). |
| 113 | 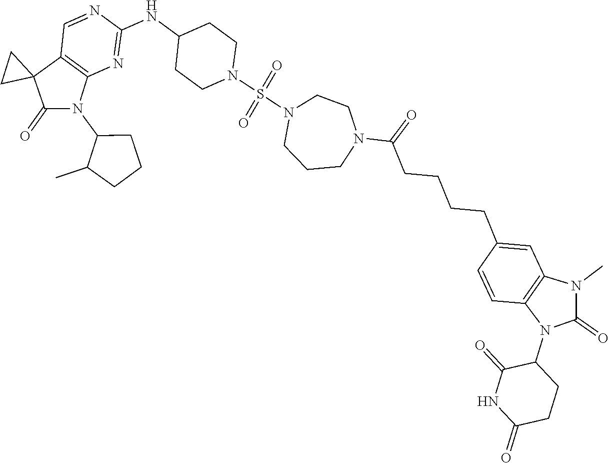 [SMILES: CC1CCCC1N1C(=O)C2(CC2)c2cnc(NC3CCN(S(=O)(=O)N4CCCN(C(=O)CCCCc5ccc6c(c5)n(C)c(=O)n6C5CCC(=O)NC5=O)CC4)CC3)nc21] | 845.3 | ¹H NMR (400 MHz, DMSO-d₆) δ 11.06 (s, 1H), 7.76 (s, 1H), 7.22-6.63 (m, 4H), 5.33 (dd, J = 12.6, 5.4 Hz, 1H), 4.86-4.05 (m, 1H), 3.77 (s, 1H), 3.61-3.39 (m, 7H), 2.94-2.74 (m, 4H), 2.74- 2.55 (m, 5H), 2.32 (d, J = 6.8 Hz, 2H), 2.26-2.09 (m, 2H), 2.08-1.86 (m, 6H), 1.86-1.64 (m, 4H), 1.64-1.31 (m, 12H), 1.23 (s, 1H), 0.81 (dd, J = 57.9, 6.7 Hz, 3H). |
| 114 | 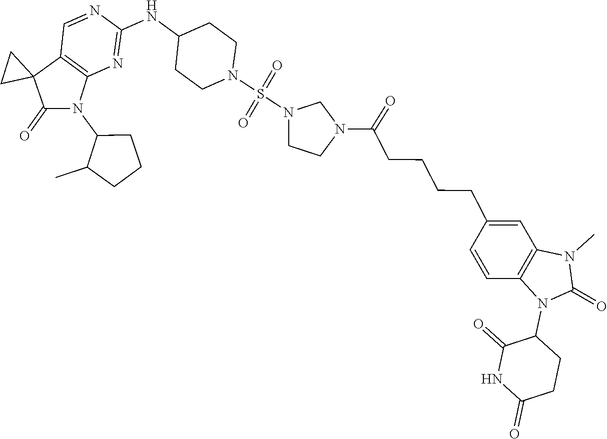 [SMILES: CC1CCCC1N1C(=O)C2(CC2)c2cnc(NC3CCN(S(=O)(=O)N4CCN(C(=O)CCCCc5ccc6c(c5)n(C)c(=O)n6C5CCC(=O)NC5=O)C4)CC3)nc21] | 816.4 | ¹H NMR (400 MHz, DMSO-d₆) δ 11.06 (s, 1H), 7.76 (d, J = 3.6 Hz, 1H), 7.21-6.96 (m, 3H), 6.90-6.84 (m, 1H), 5.38-5.27 (m, 1H), 4.82-3.78 (m, 4H), 3.71-3.39 (m, 7H), 3.09-2.82 (m, 3H), 2.78-2.58 (m, 5H), 2.36-2.11 (m, 5H), 2.03-1.74 (m, 6H), 1.72-1.41 (m, 12H), 0.91-0.67 (m, 3H). |
| 115 | 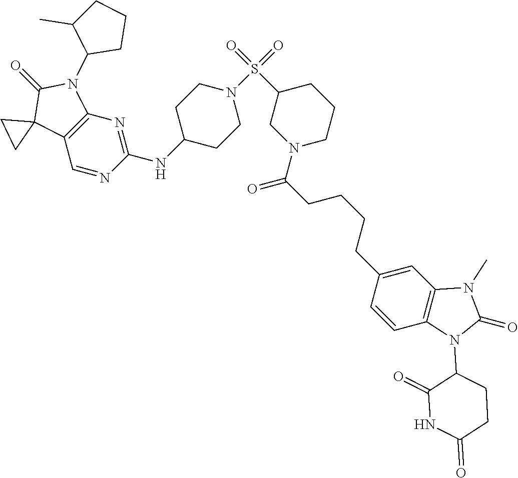 | 830.4 | ¹H NMR (300 MHz, DMSO-d6) δ 11.08 (s, 1H), 7.77 (d, J = 2.9 Hz, 1H), 7.17-6.98 (m, 3H), 6.87 (d, J = 8.1 Hz, 1H), 4.87-4.55 (m, 1H), 4.87-4.56 (m, 1H), 4.07-3.76 (m, 1H), 4.07- 3.76 (m, 2H), 3.73-3.54 (m, 2H), 3.27-3.09 (s, 1H), 3.09-2.82 (m, 4H), 2.82-2.52 (m, 6H), 2.44-2.11 (m, 4H), 2.11-1.28 (m, 22H), 1.24- 1.12 (m, 2H), 0.95-0.65 (m, 3H). |
| 116 | 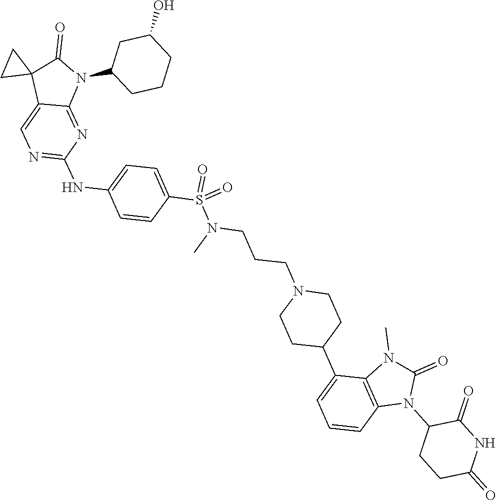 | 826.2 | ¹H NMR (400 MHz, DMSO-d₆) δ 11.09 (s, 1H), 10.06 (d, J = 1.7 Hz, 1H), 8.09-7.96 (m, 3H), 7.73-7.60 (m, 2H), 6.98 (s, 3H), 5.37 (d, J = 12.9 Hz, 1H), 4.74 (s, 1H), 4.61 (s, 1H), 4.13 (s, 1H), 3.56 (s, 3H), 3.25-3.12 (m, 1H), 3.03-2.83 (m, 5H), 2.75-2.57 (m, 5H), 2.37-2.20 (m, 4H), 2.01 (d, J = 11.1 Hz, 3H), 1.82-1.66 (m, 10H), 1.63 (d, J = 11.4 Hz, 3H), 1.54 (s, 2H), 1.42 (s, 1H). |
| 117 | 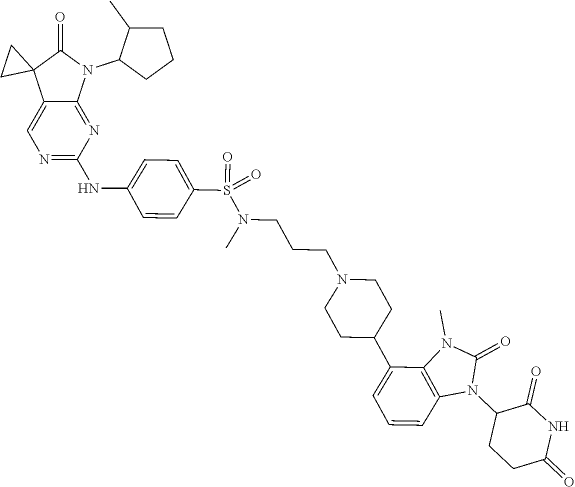 [SMILES: CC1CCCC1N1C(=O)C2(CC2)c2cnc(Nc3ccc(S(=O)(=O)N(C)CCCN4CCC(c5cccc6c5n(C)c(=O)n6C5CCC(=O)NC5=O)CC4)cc3)nc21] | 810.3 | 1H NMR (400 MHz, DMSO-d₆) δ 11.08 (s, 1H), 10.01 (d, J = 6.8 Hz, 1H), 8.10-7.92 (m, 3H), 7.71-7.62 (m, 2H), 7.05-6.92 (m, 3H), 5.42-5.29 (m, 1H), 4.91-4.21 (m, 1H), 3.56 (s, 3H), 3.23- 3.13 (m, 1H), 3.06-2.87 (m, 5H), 2.75-2.57 (m, 6H), 2.38-2.25 (m, 3H), 2.12-1.81 (m, 6H), 1.80-1.22 (m, 12H), 0.94-0.72 (m, 3H). |
| 118 | 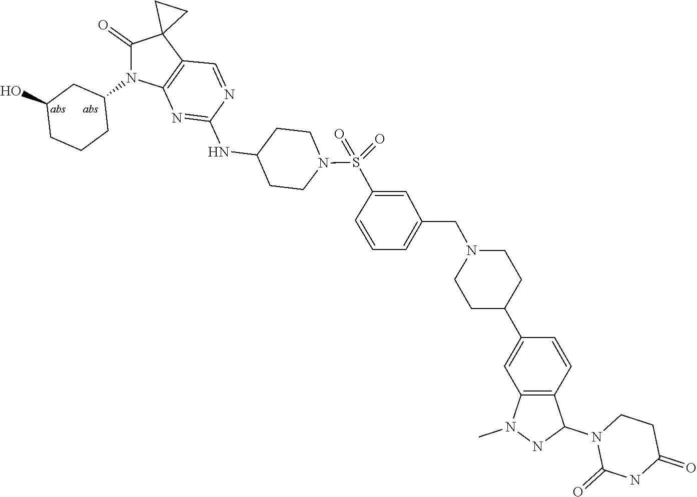 [SMILES: CN1NC(N2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)c4)CC3)cc21] | 837.4 | ¹H NMR (400 MHz, DMSO-d₆) δ 10.53 (s, 1H), 7.76-7.58 (m, 5H), 7.53 (d, J = 8.4 Hz, 1H), 7.44 (s, 1H), 7.12-6.92 (m, 2H), 4.68-4.44 (m, 2H), 4.08-3.84 (m, 6H), 3.69-3.53 (m, 4H), 2.99-2.90 (m, 2H), 2.79-2.59 (m, 3H), 2.46-2.36 (m, 3H), 2.20-2.06 (m, 3H), 1.97-1.84 (m, 2H), 1.83-1.62 (m, 5H), 1.61-1.43 (m, 9H), 1.40-1.35 (m, 3H). |
| 119 | 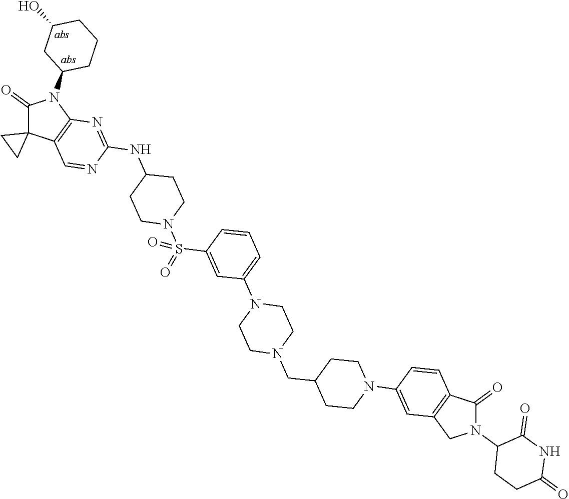 [SMILES: O=C1CCC(N2Cc3cc(N4CCC(CN5CCN(c6cccc(S(=O)(=O)N7CCC(Nc8ncc9c(n8)N([C@@H]8CCC[C@@H](O)C8)C(=O)C98CC8)CC7)c6)CC5)CC4)ccc3C2=O)C(=O)N1] | 921.4 | ¹H NMR (300 MHz, DMSO-d₆) δ 10.93 (s, 1H), 7.70 (s, 1H), 7.47 (dd, J = 16.0, 8.3 Hz, 2H), 7.28 (d, J = 8.1 Hz, 1H), 7.20-6.84 (m, 5H), 5.04 (dd, J = 13.2, 5.0 Hz, 1H), 4.70-4.54 (m, 1H), 4.52 (d, J = 2.8 Hz, 1H), 4.32 (d, J = 16.8 Hz, 1H), 4.19 (d, J = 16.9 Hz, 1H), 4.10-3.99 (m, 1H), 3.95-3.76 (m, 2H), 3.71-3.47 (m, 3H), 3.41-3.35 (m, 2H), 3.28-3.15 (m, 4H), 3.01-2.75 (m, 3H), 2.68-2.57 (m, 2H), 2.47- 2.32 (m, 4H), 2.30-2.10 (m, 3H), 2.03-1.87 (m, 3H), 1.87-1.72 (m, 3H), 1.72-1.46 (m, 9H), 1.47-1.34 (m, 2H), 1.33-1.10 (m, 4H). |
| 120 | 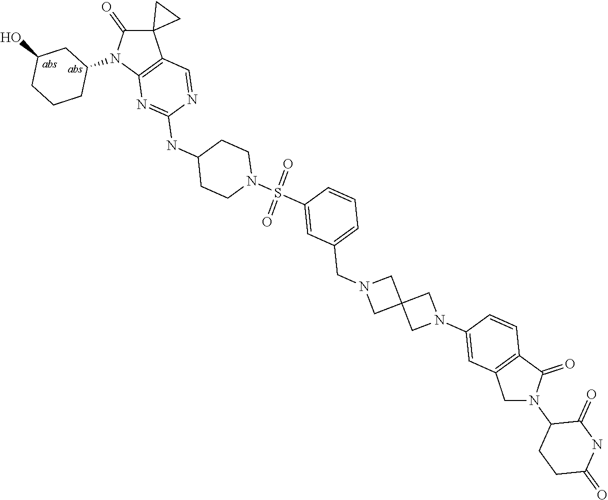 [SMILES: O=C1CCC(N2Cc3cc(N4CC5(CN(Cc6cccc(S(=O)(=O)N7CCC(Nc8ncc9c(n8)N([C@@H]8CCC[C@@H](O)C8)C(=O)C98CC8)CC7)c6)C5)C4)ccc3C2=O)C(=O)N1] | 850.3 | ¹H NMR (400 MHz, DMSO-d₆) δ 10.93 (s, 1H), 7.79-7.38 (m, 6H), 7.07 (s, 1H), 6.62-6.37 (m, 2H), 5.07-4.99 (m, 1H), 4.74-4.51 (m, 2H), 4.40-4.12 (m, 2H), 4.08-3.94 (m, 5H), 3.69- 3.47 (m, 5H), 3.37 (s, 3H), 3.02-2.80 (m, 1H), 2.63-2.54 (m, 1H), 2.43-2.24 (m, 4H), 2.17- 2.04 (m, 1H), 1.99-1.86 (m, 3H), 1.71-1.31 (m, 12H). |
| 121 | 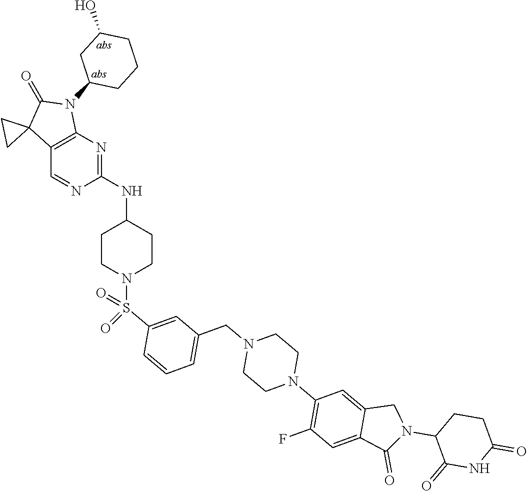 [SMILES: O=C1CCC(N2Cc3cc(N4CCN(Cc5cccc(S(=O)(=O)N6CCC(Nc7ncc8c(n7)N([C@@H]7CCC[C@@H](O)C7)C(=O)C87CC7)CC6)c5)CC4)c(F)cc3C2=O)C(=O)N1] | 856.4 | ¹H NMR (400 MHz, DMSO-d₆) δ 10.96 (s, 1H), 7.78-7.56 (m, 5H), 7.40 (d, J = 11.4 Hz, 1H), 7.22 (d, J = 7.6 Hz, 1H), 7.05 (d, J = 7.2 Hz, 1H), 5.13-5.03 (m, 1H), 4.69-4.45 (m, 2H), 4.39-4.18 (m, 2H), 4.04 (s, 1H), 3.77-3.50 (m, 5H), 3.17-3.03 (m, 4H), 2.98-2.84 (m, 1H), 2.64- 2.55 (m, 6H), 2.47-2.29 (m, 4H), 2.21-2.09 (m, 1H), 2.04-1.82 (m, 3H), 1.77-1.34 (m, 11H). |
| 122 | 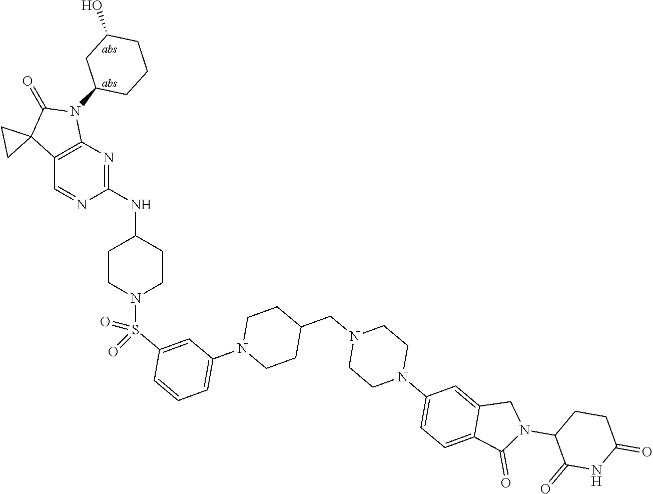 [SMILES: O=C1CCC(N2Cc3cc(N4CCN(CC5CCN(c6cccc(S(=O)(=O)N7CCC(Nc8ncc9c(n8)N([C@@H]8CCC[C@@H](O)C8)C(=O)C98CC8)CC7)c6)CC5)CC4)ccc3C2=O)C(=O)N1] | 921.5 | ¹H NMR (400 MHz, DMSO-d₆) δ 10.95 (s, 1H), 7.70 (s, 1H), 7.58-7.39 (m, 2H), 7.27 (dd, J = 8.4, 2.5 Hz, 1H), 7.16-7.00 (m, 5H), 5.05 (dd, J = 13.2, 5.1 Hz, 1H), 4.56 (d, J = 31.2 Hz, 2H), 4.41-4.14 (m, 2H), 4.04 (s, 1H), 3.77 (d, J = 12.1 Hz, 2H), 3.66-3.49 (m, 2H), 3.33-3.24 (m, 4H), 2.97-2.82 (m, 1H), 2.77 (t, J = 11.9 Hz, 2H), 2.65-2.55 (m, 2H), 2.46-2.29 (m, 5H), 2.28-2.09 (m, 4H), 2.04-1.67 (m, 8H), 1.66-1.35 (m, 10H), 1.31-1.14 (m, 3H), 0.89- 0.73 (m, 1H). |
| 123 | 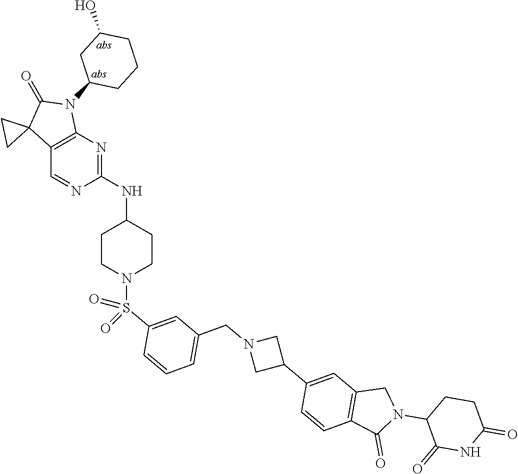 [SMILES: O=C1CCC(N2Cc3cc(C4CN(Cc5cccc(S(=O)(=O)N6CCC(Nc7ncc8c(n7)N([C@@H]7CCC[C@@H](O)C7)C(=O)C87CC7)CC6)c5)C4)ccc3C2=O)C(=O)N1] | 809.3 | ¹H NMR (400 MHz, DMSO-d₆) δ 10.98 (s, 1H), 7.79-7.38 (m, 7H), 7.06 (s, 1H), 5.15-5.05 (m, 1H), 4.68-4.24 (m, 4H), 4.03 (s, 1H), 3.88-3.79 (m, 3H), 3.74-3.54 (m, 5H), 3.25-3.17 (m, 3H), 3.00-2.80 (m, 1H), 2.64-2.53 (m, 1H), 2.47- 2.30 (m, 4H), 2.24-1.85 (m, 4H), 1.78-1.34 (m, 12H). |
| 124 | 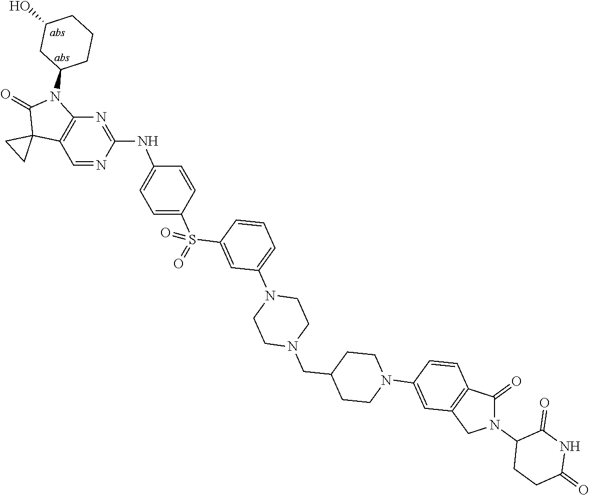 [SMILES: O=C1CCC(N2Cc3cc(N4CCC(CN5CCN(c6cccc(S(=O)(=O)c7ccc(Nc8ncc9c(n8)N([C@@H]8CCC[C@@H](O)C8)C(=O)C98CC8)cc7)c6)CC5)CC4)ccc3C2=O)C(=O)N1] | 914.5 | ¹H NMR (400 MHz, DMSO-d₆) δ 10.96 (s, 1H), 10.10 (s, 1H), 8.08-7.93 (m, 3H), 7.84 (d, J = 8.8 Hz, 2H), 7.50 (d, J = 8.4 Hz, 1H), 7.44- 7.31 (m, 2H), 7.29-7.14 (m, 2H), 7.08-7.01 (m, 2H), 5.09-4.97 (m, 1H), 4.81-4.59 (m, 2H), 4.37-4.09 (m, 3H), 3.88 (d, J = 12.4 Hz, 2H), 3.26-3.15 (m, 4H), 3.00-2.71 (m, 3H), 2.68- 2.54 (m, 2H), 2.42-2.17 (m, 7H), 2.00-1.91 (m, 1H), 1.87-1.32 (m, 13H), 1.27-1.12 (m, 3H). |
| 125 | 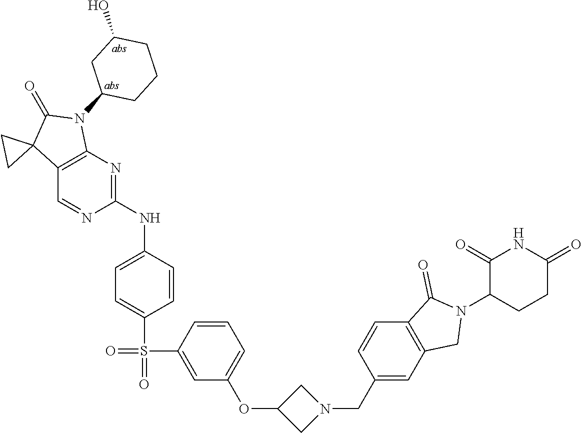 [SMILES: O=C1CCC(N2Cc3cc(CN4CC(Oc5cccc(S(=O)(=O)c6ccc(Nc7ncc8c(n7)N([C@@H]7CCC[C@@H](O)C7)C(=O)C87CC7)cc6)c5)C4)ccc3C2=O)C(=O)N1] | 818.4 | ¹H NMR (400 MHz, DMSO-d₆) δ 10.93 (s, 1H), 10.04 (s, 1H), 7.99-7.89 (m, 3H), 7.83-7.75 (m, 2H), 7.64-7.57 (m, 1H), 7.51-7.31 (m, 4H), 7.23-7.17 (m, 1H), 7.07-7.00 (m, 1H), 5.08-4.99 (m, 1H), 4.91-4.82 (m, 1H), 4.72-4.49 (m, 2H), 4.44-4.11 (m, 2H), 4.06 (s, 1H), 3.72-3.63 (m, 4H), 3.08-2.97 (m, 2H), 2.91-2.80 (m, 1H), 2.62- 2.48 (m, 1H), 2.42-2.26 (m, 2H), 2.22-2.13 (m, 1H), 1.96-1.88 (m, 1H), 1.78-1.25 (m, 10H). |
| 126 | 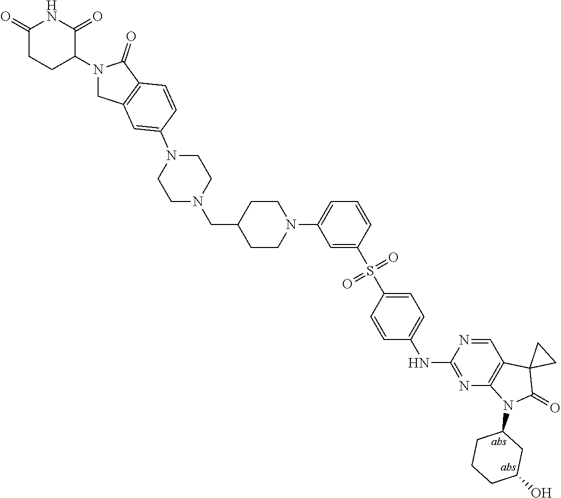 [SMILES: O=C1CCC(N2Cc3cc(N4CCN(CC5CCN(c6cccc(S(=O)(=O)c7ccc(Nc8ncc9c(n8)N([C@@H]8CCC[C@@H](O)C8)C(=O)C98CC8)cc7)c6)CC5)CC4)ccc3C2=O)C(=O)N1] | 914.4 | ¹H NMR (400 MHz, DMSO-d₆) δ 10.96 (s, 1H), 10.10 (s, 1H), 8.05-7.92 (m, 3H), 7.84 (d, J = 9.0 Hz, 2H), 7.53 (d, J = 8.3 Hz, 1H), 7.43- 7.30 (m, 2H), 7.27-7.13 (m, 2H), 7.06 (d, J = 8.4 Hz, 2H), 5.06 (dd, J = 13.3, 5.1 Hz, 1H), 4.81-4.52 (m, 2H), 4.38-4.08 (m, 3H), 3.76 (d, J = 12.5 Hz, 2H), 3.29 (t, J = 5.2 Hz, 5H), 2.91 (ddd, J = 18.4, 13.6, 5.4 Hz, 1H), 2.81- 2.69 (m, 2H), 2.63-2.54 (m, 1H), 2.48 (s, 3H), 2.39 (ddd, J = 26.4, 13.7, 9.3 Hz, 1H), 2.23 (dd, J = 20.2, 5.6 Hz, 3H), 2.01-1.91 (m, 1H), 1.87- 1.62 (m, 9H), 1.61-1.49 (m, 3H), 1.39 (td, J = 13.5, 7.2 Hz, 1H), 1.20 (dq, J = 11.8, 8.4, 6.2 Hz, 2H). |
| 127 | 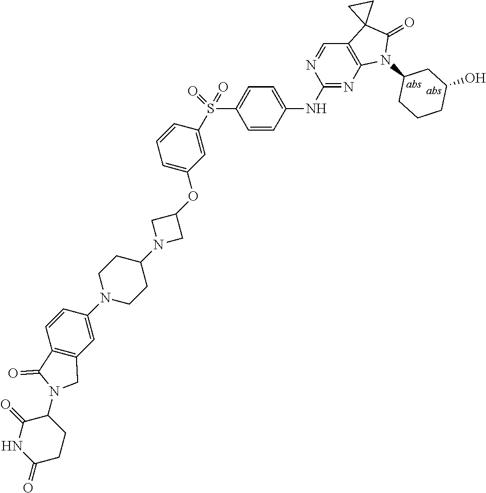 [SMILES: O=C1CCC(N2Cc3cc(N4CCC(N5CC(Oc6cccc(S(=O)(=O)c7ccc(Nc8ncc9c(n8)N([C@@H]8CCC[C@@H](O)C8)C(=O)C98CC8)cc7)c6)C5)CC4)ccc3C2=O)C(=O)N1] | 887.3 | ¹H NMR (400 MHz, DMSO-d₆) δ 10.94 (s, 1H), 10.12 (s, 1H), 8.23-7.94 (m, 3H), 7.86 (d, J = 8.7 Hz, 2H), 7.59-7.45 (m, 3H), 7.25 (t, J = 2.0 Hz, 1H), 7.13 (dt, J = 7.0, 2.6 Hz, 1H), 7.08- 6.96 (m, 2H), 5.04 (dd, J = 13.3, 5.1 Hz, 1H), 4.89 (p, J = 5.6 Hz, 1H), 4.79-4.66 (m, 1H), 4.62 (d, J = 2.7 Hz, 1H), 4.31 (d, J = 16.9 Hz, 1H), 4.19 (d, J = 16.8 Hz, 1H), 4.12 (s, 1H), 3.80- 3.60 (m, 3H), 3.09-2.83 (m, 4H), 2.69-2.54 (m, 1H), 2.48-2.15 (m, 4H), 2.03-1.89 (m, 1H), 1.84-1.61 (m, 8H), 1.53 (q, J = 3.8 Hz, 3H), 1.39 (t, J = 13.4 Hz, 1H), 1.33-1.14 (m, 3H), 0.85 (t, J = 7.2 Hz, 1H). |
| 128 | 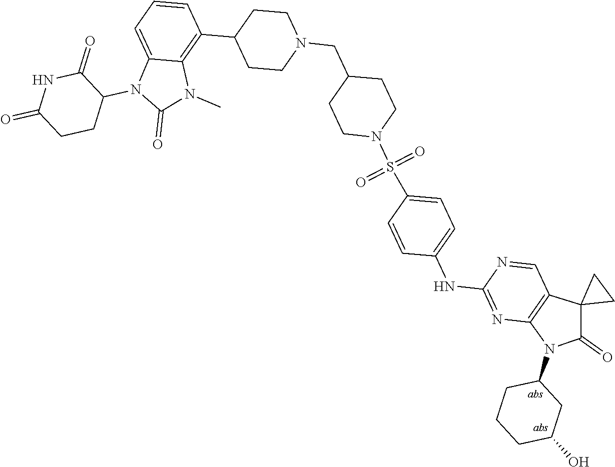 [SMILES: Cn1c(=O)n(C2CCC(=O)NC2=O)c2cccc(C3CCN(CC4CCN(S(=O)(=O)c5ccc(Nc6ncc7c(n6)N([C@@H]6CCC[C@@H](O)C6)C(=O)C76CC6)cc5)CC4)CC3)c21] | 852.3 | 1H NMR (400 MHz, DMSO-d6) δ 11.10 (s, 1H), 10.08 (s, 1H), 8.13-7.91 (m, 3H), 7.62 (d, J = 8.4 Hz, 2H), 7.22-6.91 (m, 3H), 5.41-5.31 (m, 1H), 4.85-4.54 (m, 2H), 4.13 (s, 1H), 3.66-3.49 (m, 6H), 3.22-3.11 (m, 2H), 2.94-2.81 (m, 3H), 2.75-2.57 (m, 4H), 2.32-2.09 (m, 6H), 2.03-1.91 (m, 4H), 1.82-1.61 (m, 12H), 1.61-1.36 (m, 5H), 1.18-1.02 (m, 2H). |
| 129 | 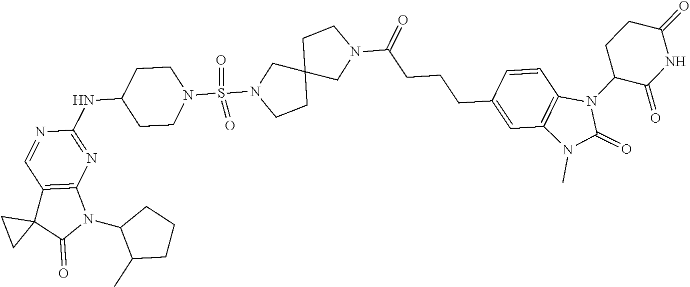 [SMILES: CC1CCCC1N1C(=O)C2(CC2)c2cnc(NC3CCN(S(=O)(=O)N4CCC5(CCN(C(=O)CCCc6ccc7c(c6)n(C)c(=O)n7C6CCC(=O)NC6=O)C5)C4)CC3)nc21] | 857.4 | 1H NMR (400 MHz, DMSO-d6) δ 11.09 (s, 1H), 7.91-7.64 (m, 1H), 7.19-6.96 (m, 3H), 6.96-6.78 (m, 1H), 5.34 (dd, J = 12.8, 5.4 Hz, 1H), 4.88- 4.11 (m, 1H), 3.81 (s, 1H), 3.65-3.51 (m, 2H), 3.51-3.44 (m, 1H), 3.38 (d, J = 8.8 Hz, 1H), 3.30-3.06 (m, 5H), 2.83-2.99 (m, 3H), 2.78-2.55 (m, 3H), 2.13-2.3 (m, 2H), 2.06-1.64 (m, 14H), 1.61-1.38 (m, 8H), 1.31-1.16 (m, 3H), 0.88 (d, J = 6.6 Hz, 2H), 0.74 (d, J = 7.0 Hz, 1H). |
| 130 | 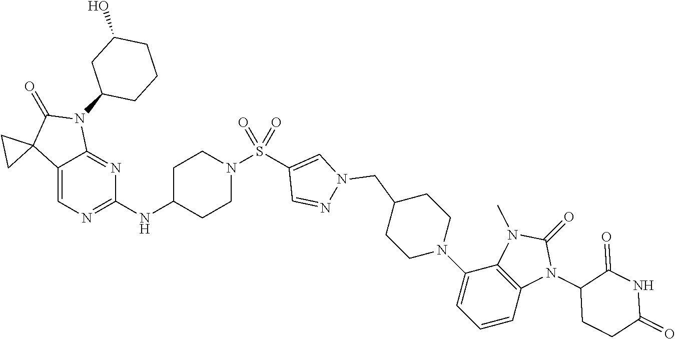 [SMILES: Cn1c(=O)n(C2CCC(=O)NC2=O)c2cccc(N3CCC(Cn4cc(S(=O)(=O)N5CCC(Nc6ncc7c(n6)N([C@@H]6CCC[C@@H](O)C6)C(=O)C76CC6)CC5)cn4)CC3)c21] | 842.4 | 1H NMR (400 MHz, DMSO-d6) δ 11.06 (s, 1H), 8.41 (s, 1H), 7.83 (s, 1H), 7.68 (s, 1H), 7.12- 7.02 (m, 1H), 6.97 (t, J = 7.9 Hz, 1H), 6.88 (t, J = 8.7 Hz, 2H), 5.34 (dd, J = 12.7, 5.4 Hz, 1H), 4.61 (t, J = 12.6 Hz, 1H), 4.51 (d, J = 2.7 Hz, 1H), 4.17 (d, J = 6.8 Hz, 2H), 4.10-4.02 (m, 1H), 3.62 (s, 4H), 3.50 (d, J = 10.9 Hz, 2H), 3.11 (d, J = 11.3 Hz, 2H), 2.88 (t, J = 14.9 Hz, 1H), 2.74-2.57 (m, 4H), 2.47-2.34 (m, 3H), 2.17 (d, J = 12.6 Hz, 1H), 1.98 (d, J = 13.8 Hz, 4H), 1.71 (d, J = 13.1 Hz, 1H), 1.59 (d, J = 15.1 Hz, 7H), 1.51 (d, J = 3.6 Hz, 3H), 1.46 (d, J = 11.6 Hz, 2H), 1.40 (q, J = 4.2, 3.7 Hz, 2H), 1.27 (m, 1H) |
| 131 | 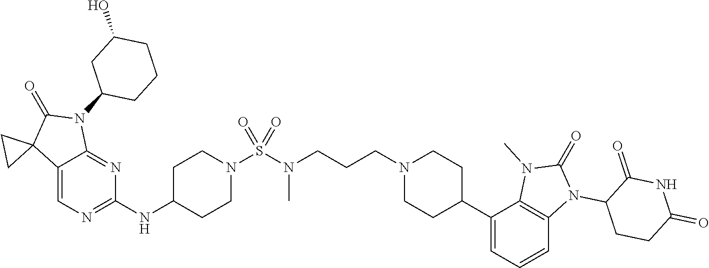 [SMILES: CN(CCCN1CCC(c2cccc3c2n(C)c(=O)n3C2CCC(=O)NC2=O)CC1)S(=O)(=O)N1CCC(Nc2ncc3c(n2)N([C@@H]2CCC[C@@H](O)C2)C(=O)C32CC2)CC1] | 833.4 | 1H NMR (400 MHz, DMSO-d6) δ 11.08 (s, 1H), 7.74 (s, 1H), 7.17-6.94 (m, 4H), 5.37 (dd, J = 12.5, 5.4 Hz, 1H), 4.70-4.57 (m, 1H), 4.54 (d, J = 2.7 Hz, 1H), 4.10 (s, 1H), 3.80 (s, 1H), 3.58 (s, 5H), 3.27-3.12 (m, 3H), 3.00 (d, J = 10.7 Hz, 2H), 2.89 (q, J = 9.6, 8.2 Hz, 3H), 2.79 (s, 3H), 2.68 (dtd, J = 30.9, 13.2, 3.8 Hz, 2H), 2.55- 2.52 (m, 1H), 2.35 (t, J = 7.0 Hz, 2H), 2.21 (s, 1H), 2.12-1.88 (m, 5H), 1.85-1.67 (m, 7H), 1.67-1.47 (m, 7H), 1.46-1.30 (m, 3H). |
| 132 | 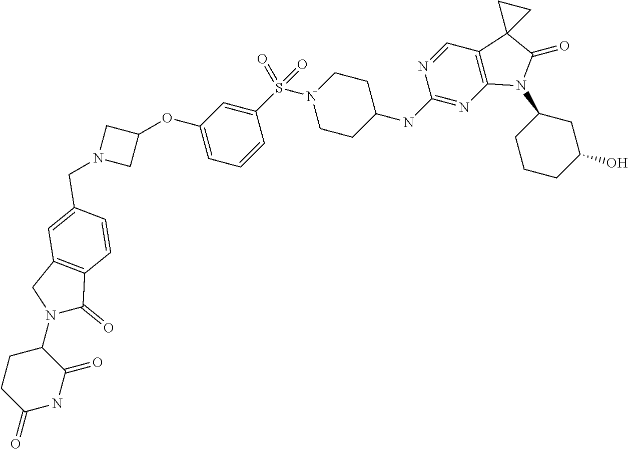 [SMILES: O=C1CCC(N2Cc3cc(CN4CC(Oc5cccc(S(=O)(=O)N6CCC(Nc7ncc8c(n7)N([C@@H]7CCC[C@@H](O)C7)C(=O)C87CC7)CC6)c5)C4)ccc3C2=O)C(=O)N1] | 825.4 | 1H NMR (300 MHz, DMSO-d6) δ 11.00 (s, 1H), 7.89-7.63 (m, 2H), 7.62-7.51 (m, 2H), 7.44 (d, J = 7.6 Hz, 1H), 7.38-7.29 (m, 1H), 7.26- 7.15 (m, 1H), 7.07 (t, J = 2.1 Hz, 2H), 5.11 (dd, J = 13.2, 5.1 Hz, 1H), 4.96 (t, J = 5.5 Hz, 1H), 4.57 (d, J = 21.4 Hz, 2H), 4.45 (d, J = 17.4 Hz, 1H), 4.31 (d, J = 17.3 Hz, 1H), 4.05 (s, 1H), 3.78 (d, J = 4.6 Hz, 4H), 3.56 (d, J = 11.8 Hz, 3H), 3.15 (t, J = 6.6 Hz, 2H), 2.92 (ddd, J = 17.8, 13.4, 5.3 Hz, 1H), 2.59 (d, J = 18.1 Hz, 1H), 2.47 2.30 (m, 3H), 2.13 (dd, J = 21.9, 10.1 Hz, 1H), 2.06-1.83 (m, 3H), 1.79-1.46 (m, 9H), 1.41 (q, J = 4.2, 3.6 Hz, 2H), 1.24 (s, 2H), 0.90-0.79 (m, 1H). |
| 133 | 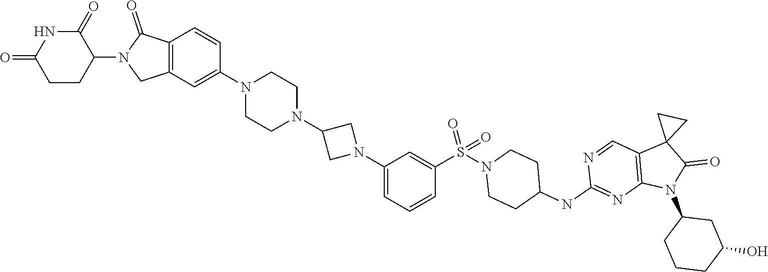 [SMILES: O=C1CCC(N2Cc3cc(N4CCN(C5CN(c6cccc(S(=O)(=O)N7CCC(Nc8ncc9c(n8)N([C@@H]8CCC[C@@H](O)C8)C(=O)C98CC8)CC7)c6)C5)CC4)ccc3C2=O)C(=O)N1] | 879.5 | 1H NMR (400 MHz, DMSO-d6) δ 10.93 (s, 1H), 7.69 (s, 1H), 7.53 (d, J = 8.4 Hz, 1H), 7.42 (t, J = 7.9 Hz, 1H), 7.15-7.01 (m, 3H), 6.99 (d, J = 7.7 Hz, 1H), 6.73 (d, J = 8.2 Hz, 1H), 6.65 (s, 1H), 5.04 (dd, J = 13.6, 5.1 Hz, 1H), 4.60 (t, J = 12.7 Hz, 1H), 4.54-4.45 (m, 1H), 4.33 (d, J = 16.9 Hz, 1H), 4.21 (d, J = 16.8 Hz, 1H), 4.09-3.93 (m, 3H), 3.75 (t, J = 6.3 Hz, 3H), 3.66-3.45 (m, 4H), 3.40-3.33 (m, 2H), 2.99-2.80 (m, 1H), 2.67-2.53 (m, 4H), 2.47-2.28 (m, 4H), 2.25- 2.08 (m, 1H), 2.03-1.84 (m, 3H), 1.80-1.16 (m, 14H). |
| 134 | 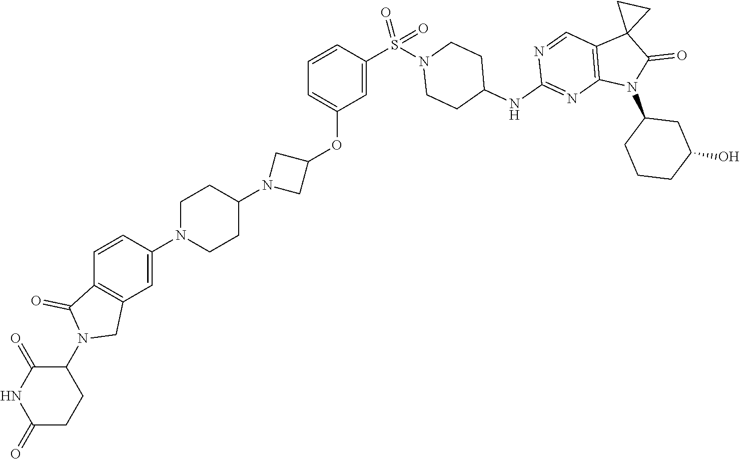 [SMILES: O=C1CCC(N2Cc3cc(N4CCC(N5CC(Oc6cccc(S(=O)(=O)N7CCC(Nc8ncc9c(n8)N([C@@H]8CCC[C@@H](O)C8)C(=O)C98CC8)CC7)c6)C5)CC4)ccc3C2=O)C(=O)N1] | 894.5 | 1H NMR (300 MHz, DMSO-d6) δ 10.96 (s, 1H), 7.71 (s, 1H), 7.58 (t, J = 8.0 Hz, 1H), 7.50 (d, J = 8.4 Hz, 1H), 7.33 (d, J = 7.7 Hz, 1H), 7.23 (dd, J = 8.2, 2.5 Hz, 1H), 7.15-6.99 (m, 4H), 5.05 (dd, J = 13.2, 5.1 Hz, 1H), 4.93 (t, J = 5.5 Hz, 1H), 4.68-4.51 (m, 2H), 4.32 (d, J = 16.9 Hz, 1H), 4.19 (d, J = 16.9 Hz, 1H), 4.05 (s, 1H), 3.88 3.49 (m, 7H), 3.13-2.80 (m, 5H), 2.61 (s, 1H), 2.48-2.28 (m, 5H), 2.17 (d, J = 11.9 Hz, 1H), 1.95 (q, J = 9.8, 7.6 Hz, 3H), 1.76 (d, J = 11.7 Hz, 3H), 1.67-1.45 (m, 8H), 1.41 (q, J = 4.2, 3.6 Hz, 2H), 1.29 (d, J = 10.3 Hz, 2H), 1.24 (s, 1H). |
| 135 | 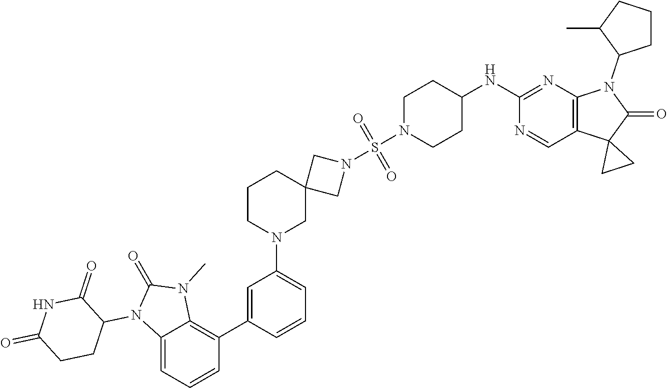 [SMILES: CC1CCCC1N1C(=O)C2(CC2)c2cnc(NC3CCN(S(=O)(=O)N4CC5(CCCN(c6cccc(-c7cccc8c7n(C)c(=O)n8C7CCC(=O)NC7=O)c6)C5)C4)CC3)nc21] | 870.4 | 1H NMR (400 MHz, DMSO-d6) δ 11.08 (s, 1H), 7.76 (d, J = 3.7 Hz, 1H), 7.07 (s, 1H), 7.00- 6.81 (m, 3H), 5.35 (dd, J = 12.7, 5.3 Hz, 1H), 4.47 (dd, J = 234.5, 8.8 Hz, 1H), 3.80 (s, 1H), 3.61 (s, 3H), 3.48 (s, 3H), 3.09 (dd, J = 54.7, 10.4 Hz, 4H), 2.89 (t, J = 12.7 Hz, 4H), 2.78- 2.54 (m, 3H), 2.38-2.06 (m, 2H), 2.03-1.61 (m, 11H), 1.59-1.30 (m, 15H), 1.23 (s, 1H), 0.80 (dd, J = 56.8, 6.8 Hz, 4H). |
| 136 | 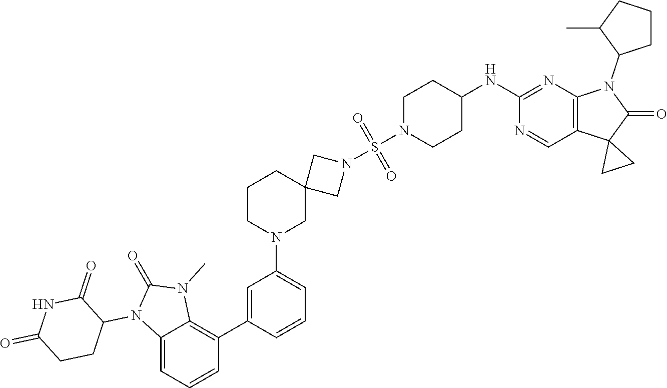 [SMILES: CC1CCCC1N1C(=O)C2(CC2)c2cnc(NC3CCN(S(=O)(=O)N4CC5(CCCN(c6cccc(-c7cccc8c7n(C)c(=O)n8C7CCC(=O)NC7=O)c6)C5)C4)CC3)nc21] | 863.3 | 1H NMR (400 MHz, DMSO-d6) δ 11.14 (s, 1H), 7.76 (d, J = 3.7 Hz, 1H), 7.31 (t, J = 7.8 Hz, 1H), 7.15 (d, J = 7.9 Hz, 1H), 7.07 (dd, J = 9.0, 6.6 Hz, 3H), 7.00 (s, 1H), 6.91 (d, J = 7.6 Hz, 1H), 6.86 (d, J = 7.4 Hz, 1H), 5.52-5.36 (m, 1H), 4.20 (d, J = 9.1 Hz, 1H), 3.81 (s, 1H), 3.61- 3.56 (m, 4H), 3.26 (s, 2H), 3.09 (d, J = 6.5 Hz, 2H), 2.97-2.72 (m, 7H), 2.65 (d, J = 4.2 Hz, 1H), 2.64-2.57 (m, 1H), 2.17 (dt, J = 16.1, 7.2 Hz, 1H), 2.05 (dd, J = 14.3, 7.8 Hz, 1H), 2.01- 1.75 (m, 6H), 1.75-1.66 (m, 3H), 1.66-1.53 (m, 5H), 1.52-1.40 (m, 4H), 1.23 (q, J = 10.8, 9.5 Hz, 1H), 0.80 (dd, J = 56.8, 6.8 Hz, 3H). |
| 137 | 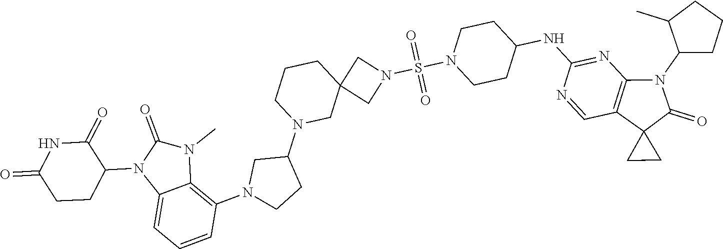 [SMILES: CC1CCCC1N1C(=O)C2(CC2)c2cnc(NC3CCN(S(=O)(=O)N4CC5(CCCN(C6CCN(c7cccc8c7n(C)c(=O)n8C7CCC(=O)NC7=O)C6)C5)C4)CC3)nc21] | 856.5 | 1H NMR (400 MHz, DMSO-d6) δ 11.10 (s, 1H), 7.76 (d, J = 3.6 Hz, 1H), 7.10 (s, 1H), 7.02- 6.93 (m, 2H), 6.86 (d, J = 4.1 Hz, 1H), 5.35 (dd, J = 12.6, 5.3 Hz, 1H), 4.47 (dd, J = 234.9, 8.9 Hz, 1H), 3.81 (s, 1H), 3.68-3.45 (m, 9H), 3.24 (t, J = 8.1 Hz, 1H), 3.08 (d, J = 11.2 Hz, 3H), 2.90 (q, J = 12.9, 10.8 Hz, 4H), 2.77-2.57 (m, 4H), 2.37 (d, J = 30.5 Hz, 2H), 2.25-2.05 (m, 2H), 2.05-1.73 (m, 8H), 1.67 (s, 1H), 1.63- 1.39 (m, 11H), 0.80 (dd, J = 57.3, 6.8 Hz, 3H). |
| 138 | 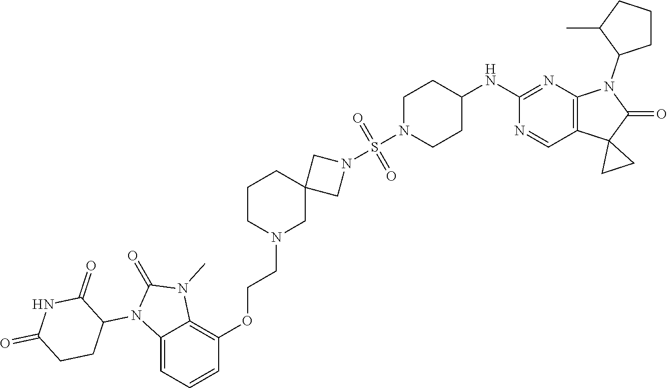 [SMILES: CC1CCCC1N1C(=O)C2(CC2)c2cnc(NC3CCN(S(=O)(=O)N4CC5(CCCN(CCOc6cccc7c6n(C)c(=O)n7C6CCC(=O)NC6=O)C5)C4)CC3)nc21] | 831.4 | 1H NMR (300 MHz, DMSO-d6) δ 11.07 (s, 1H), 7.76 (d, J = 2.7 Hz, 1H), 7.08 (s, 1H), 6.95 (t, J = 8.1 Hz, 1H), 6.77 (dd, J = 14.0, 8.1 Hz, 2H), 5.33 (dd, J = 12.6, 5.4 Hz, 1H), 4.76 (q, J = 9.1, 8.2 Hz, 1H), 4.28-4.09 (m, 2H), 3.89-3.69 (m, 1H), 3.61-3.41 (m, 9H), 2.95-2.74 (m, 5H), 2.71-2.54 (m, 4H), 2.46-2.31 (m, 2H), 2.31- 2.11 (m, 1H), 2.03-1.62 (m, 7H), 1.61-1.33 (m, 11H), 1.30-1.06 (m, 1H), 0.80 (dd, J = 42.6, 6.8 Hz, 3H). |
| 139 | 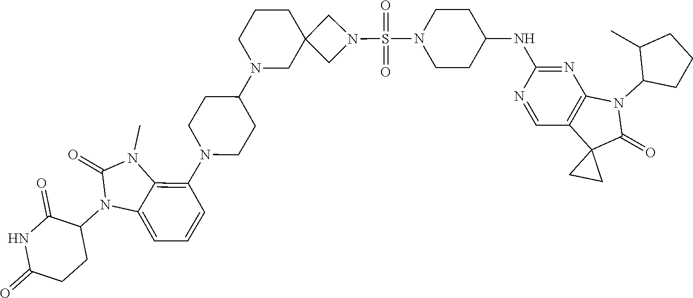 [SMILES: CC1CCCC1N1C(=O)C2(CC2)c2cnc(NC3CCN(S(=O)(=O)N4CC5(CCCN(C6CCN(c7cccc8c7n(C)c(=O)n8C7CCC(=O)NC7=O)CC6)C5)C4)CC3)nc21] | 870.5 | 1H NMR (400 MHz, DMSO-d6) δ 11.09 (s, 1H), 7.76 (d, J = 3.6 Hz, 1H), 7.09 (s, 1H), 7.00- 6.84 (m, 3H), 5.35 (dd, J = 12.6, 5.4 Hz, 1H), 4.47 (dd, J = 235.1, 8.3 Hz, 1H), 3.82 (s, 1H), 3.69-3.47 (m, H), 3.15 (d, J = 10.9 Hz, 2H), 2.91 (t, J = 11.7 Hz, 3H), 2.80-2.55 (m, 7H), 2.47 (s, 3H), 2.18 (dt, J = 15.8, 8.0 Hz, 1H), 2.04- 1.86 (m, 4H), 1.86-1.61(m, 6H), 1.56 (d, J = 4.0 Hz, 4H), 1.53-1.36 (m, 7H), 0.80 (dd, J = 57.1, 6.8 Hz, 3H). |
| 140 | 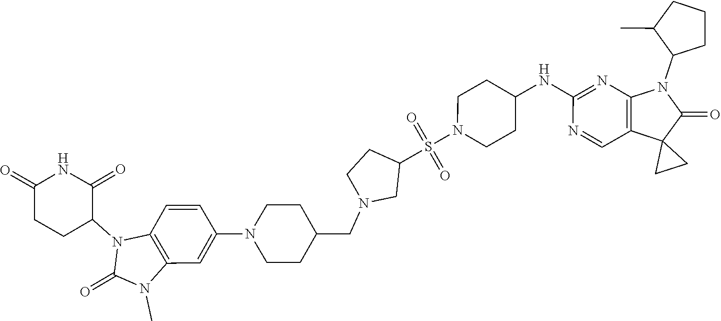 [SMILES: CC1CCCC1N1C(=O)C2(CC2)c2cnc(NC3CCN(S(=O)(=O)C4CCN(CC5CCN(c6ccc7c(c6)n(C)c(=O)n7C6CCC(=O)NC6=O)CC5)C4)CC3)nc21] | 829.4 | 1H NMR (400 MHz, DMSO-d6) δ 11.06 (s, 1H), 7.75 (d, J = 3.4 Hz, 1H), 7.20-6.98 (m, 1H), 6.92 (d, J = 8.6 Hz, 1H), 6.81 (d, J = 2.2 Hz, 1H), 6.62 (dd, J = 8.6, 2.2 Hz, 1H), 5.28 (dd, J = 12.9, 5.3 Hz, 1H), 4.82-4.05 (m, 1H), 3.86 (s, 2H), 3.71-3.53 (m, 4H), 3.29 (s, 3H), 3.04- 2.81 (m, 4H), 2.75-2.56 (m, 6H), 2.49-2.42 (m, 1H), 2.32 (d, J = 7.2 Hz, 2H), 2.24-2.07 (m, 2H), 2.07-1.74 (m, 9H), 1.73-1.37 (m, 9H), 1.25 (d, J = 12.4 Hz, 3H), 0.81 (dd, J = 56.8, 6.8 Hz, 3H). |
| 141 | 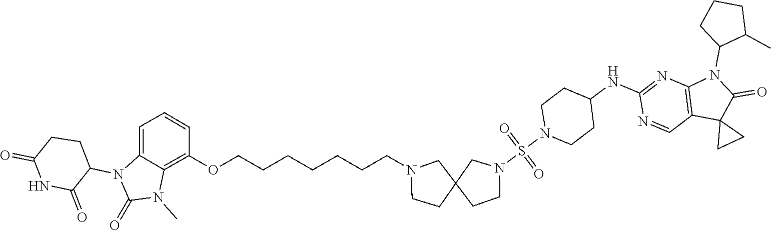 [SMILES: CC1CCCC1N1C(=O)C2(CC2)c2cnc(NC3CCN(S(=O)(=O)N4CCC5(CCN(CCCCCCCOc6cccc7c6n(C)c(=O)n7C6CCC(=O)NC6=O)C5)C4)CC3)nc21] | 901.4 | 1H NMR (400 MHz, DMSO-d6) δ 11.07 (s, 1H), 7.75 (d, J = 3.6 Hz, 1H), 7.13-6.89 (m, 2H), 6.74 (d, J = 8.2 Hz, 2H), 5.32 (dd, J = 12.6, 5.4 Hz, 2H), 4.86-4.68 (m, 2H), 4.26-3.95 (m, 4H), 3.75 (d, J = 40.8 Hz, 2H), 3.52 (s, 5H), 3.26 (t, J = 7.4 Hz, 2H), 3.17 (d, J = 9.2 Hz, 1H), 3.07 (d, J = 9.2 Hz, 1H), 2.88 (q, J = 11.7, 9.7 Hz, 3H), 2.59 (d, J = 34.0 Hz, 1H), 2.47-2.28 (m, 4H), 2.18 (ddd, J = 16.0, 9.1, 6.7 Hz, 1H), 2.03- 1.62 (m, 11H), 1.59-1.21 (m, 16H), 0.81 (dd, J = 57.5, 6.8 Hz, 3H). |
| 142 | 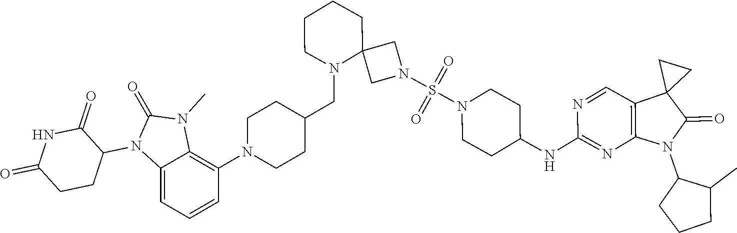 [SMILES: CC1CCCC1N1C(=O)C2(CC2)c2cnc(NC3CCN(S(=O)(=O)N4CC5(CCCCN5CC5CCN(c6cccc7c6n(C)c(=O)n7C6CCC(=O)NC6=O)CC5)C4)CC3)nc21] | 884.5 | 1H NMR (400 MHz, DMSO-d6) δ 11.07 (s, 1H), 7.75 (d, J = 3.3 Hz, 1H), 7.07 (s, 1H), 6.97 (t, J = 7.9 Hz, 1H), 6.87 (dd, J = 14.9, 7.8 Hz, 2H), 5.34 (dd, J = 12.7, 5.4 Hz, 1H), 4.48 (dt, J = 234.5, 8.8 Hz, 1H), 3.80 (d, J = 8.0 Hz, 3H), 3.62 (s, 5H), 3.51 (d, J = 8.0 Hz, 2H), 3.12 (d, J = 10.9 Hz, 2H), 2.99-2.82 (m, 3H), 2.78-2.57 (m, 5H), 2.43 (d, J = 6.3 Hz, 2H), 2.28-2.11 (m, 1H), 2.05-1.75 (m, 8H), 1.70 (d, J = 6.3 Hz, 4H), 1.59-1.39 (m, 12H), 1.39-1.32 (m, 3H), 0.80 (dd, J = 57.4, 6.8 Hz, 3H). |
| 143 | 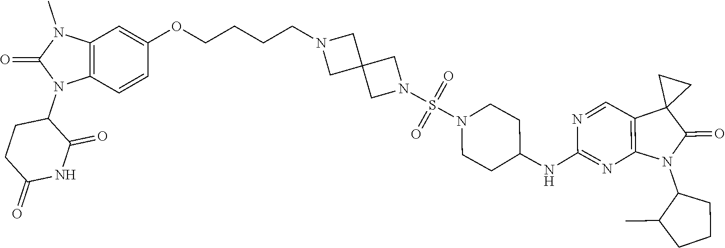 [SMILES: CC1CCCC1N1C(=O)C2(CC2)c2cnc(NC3CCN(S(=O)(=O)N4CC5(CN(CCCCOc6ccc7c(c6)n(C)c(=O)n7C6CCC(=O)NC6=O)C5)C4)CC3)nc21] | 831.5 | 1H NMR (300 MHz, DMSO-d6) δ 7.74 (d, J = 3.0 Hz, 1H), 7.03 (d, J = 8.5 Hz, 1H), 6.93 (d, J = 2.3 Hz, 1H), 6.71 (dd, J = 8.6, 2.3 Hz, 1H), 5.37-5.23 (m, 1H), 4.48 (dd, J = 168.2, 8.6 Hz, 1H), 4.15 (s, 2H), 3.89-3.75 (m, 4H), 3.69- 3.53 (m, 3H), 3.32 (s, 3H), 2.92 (t, J = 11.4 Hz, 3H), 2.80 (s, 2H), 2.73-2.57 (m, 3H), 2.48 (d, J = 5.7 Hz, 4H), 2.30-2.09 (m, 1H), 2.09-1.72 (m, 6H), 1.67 (d, J = 5.4 Hz, 5H), 1.62-1.39 (m, 7H), 0.79 (dd, J = 43.2, 6.7 Hz, 3H). |
| 144 | 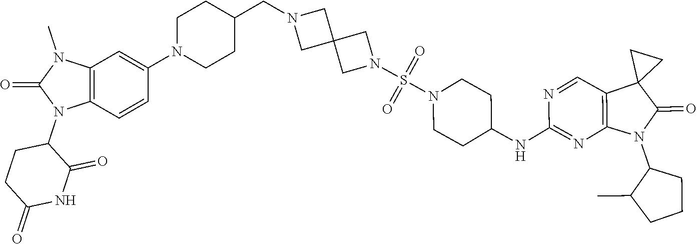 [SMILES: CC1CCCC1N1C(=O)C2(CC2)c2cnc(NC3CCN(S(=O)(=O)N4CC5(CN(CC6CCN(c7ccc8c(c7)n(C)c(=O)n8C7CCC(=O)NC7=O)CC6)C5)C4)CC3)nc21] | 856.3 | 1H NMR (400 MHz, DMSO-d6) δ 11.06 (s, 1H), 7.76 (d, J = 3.5 Hz, 1H), 7.08 (s, 1H), 6.92 (d, J = 8.6 Hz, 1H), 6.81 (d, J = 2.2 Hz, 1H), 6.61 (dd, J = 8.7, 2.2 Hz, 1H), 5.28 (dd, J = 12.9, 5.3 Hz, 1H), 4.48 (dd, J = 235.4, 9.0 Hz, 1H), 3.87 (s, 5H), 3.54 (d, J = 11.3 Hz, 4H), 3.30 (s, 3H), 3.24 (s, 4H), 2.89 (t, J = 11.4 Hz, 3H), 2.73-2.53 (m, 3H), 2.28-2.14 (m, 3H), 2.02-1.90 (m, 3H), 1.90-1.80 (m, 3H), 1.73 (d, J = 15.0 Hz, 3H), 1.61-1.54 (m, 2H), 1.54-1.41 (m, 5H), 1.33 (d, J = 8.6 Hz, 1H), 1.23 (q, J = 10.9, 9.8 Hz, 3H), 0.81 (dd, J = 57.3, 6.8 Hz, 3H). |
| 145 | 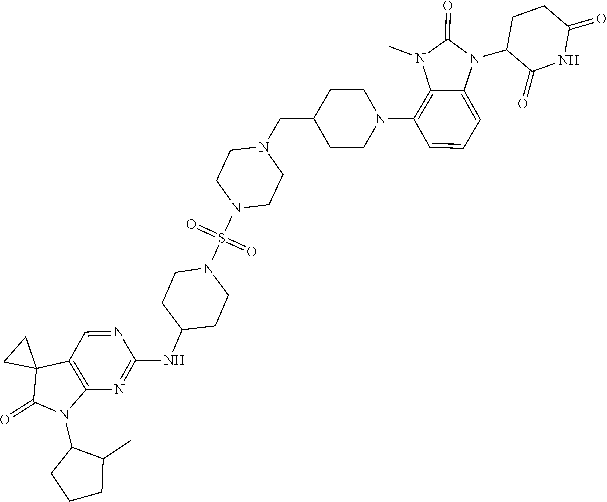 [SMILES: CC1CCCC1N1C(=O)C2(CC2)c2cnc(NC3CCN(S(=O)(=O)N4CCN(CC5CCN(c6cccc7c6n(C)c(=O)n7C6CCC(=O)NC6=O)CC5)CC4)CC3)nc21] | 844.5 | 1H NMR (400 MHz, DMSO-d6) δ 11.08 (s, 1H), 7.76 (d, J = 3.7 Hz, 1H), 7.08 (s, 1H), 6.97 (t, J = 7.9 Hz, 1H), 6.88 (dd, J = 12.1, 7.9 Hz, 2H), 5.34 (dd, J = 12.7, 5.4 Hz, 1H), 4.49 (dt, J = 235.2, 8.8 Hz, 1H), 3.81 (d, J = 14.5 Hz, 1H), 3.60 (d, J = 20.3 Hz, 5H), 3.23-3.05 (m, 6H), 3.02-2.82 (m, 3H), 2.76-2.57 (m, 5H), 2.43 (t, J = 4.8 Hz, 4H), 2.30-2.11 (m, 3H), 2.04-1.74 (m, 8H), 1.67 (d, J = 9.4 Hz, 2H), 1.60-1.38 (m, 7H), 1.38-1.15 (m, 2H), 0.88 (d, J = 6.6 Hz, 1H), 0.74 (d, J = 6.9 Hz, 2H). |
| 146 | 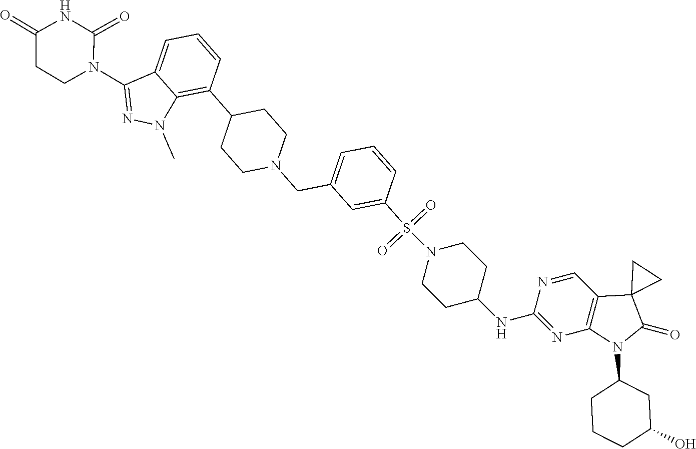 [SMILES: Cn1nc(N2CCC(=O)NC2=O)c2cccc(C3CCN(Cc4cccc(S(=O)(=O)N5CCC(Nc6ncc7c(n6)N([C@@H]6CCC[C@@H](O)C6)C(=O)C76CC6)CC5)c4)CC3)c21] | 837.4 | 1H NMR (400 MHz, DMSO-d6) δ 10.55 (s, 1H), 7.82-7.59 (m, 5H), 7.45 (d, J = 8.0 Hz, 1H), 7.26 (d, J = 7.2 Hz, 1H), 7.15-6.93 (m, 2H), 4.55 (d, J = 25.2 Hz, 2H), 4.19 (s, 3H), 4.03 (s, 1H), 3.86 (t, J = 6.6 Hz, 2H), 3.68 (s, 2H), 3.59 (d, J = 11.5 Hz, 3H), 2.97 (d, J = 10.8 Hz, 2H), 2.75 (t, J = 6.7 Hz, 2H), 2.44 (q, J = 12.2, 11.5 Hz, 3H), 2.30-2.07 (m, 4H), 1.99-1.66 (m, 7H), 1.66-1.43 (m, 8H), 1.39 (q, J = 4.0, 3.6 Hz, 3H). |
| 147 | 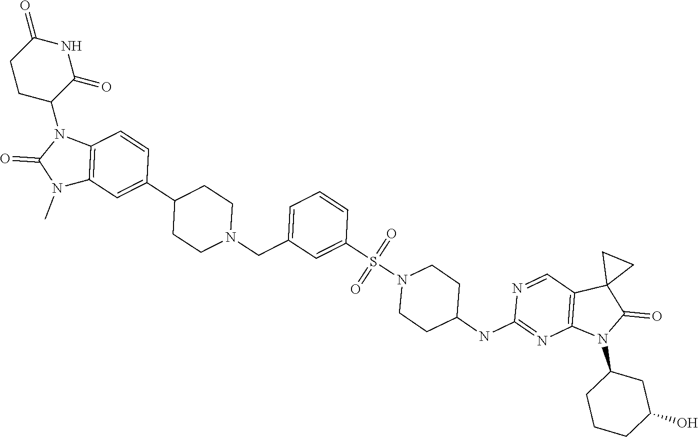 [SMILES: Cn1c(=O)n(C2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)c4)CC3)cc21] | 852.5 | 1H NMR (400 MHz, DMSO-d6) δ 11.06 (s, 1H), 7.85-7.49 (m, 5H), 7.18-6.87 (m, 4H), 5.32 (dd, J = 12.7, 5.4 Hz, 1H), 4.66-4.42 (m, 2H), 4.03 (s, 1H), 3.61 (d, J = 16.3 Hz, 6H), 3.32 (s, 2H), 2.99-2.82 (m, 3H), 2.76-2.53 (m, 3H), 2.44 (d, J = 22.9 Hz, 3H), 2.13 (td, J = 19.7, 17.8, 11.1 Hz, 3H), 2.04-1.84 (m, 3H), 1.74 (d, J = 9.0 Hz, 6H), 1.66-1.43 (m, 8H), 1.38 (q, J = 4.1, 3.6 Hz, 2H). |
| 148 | 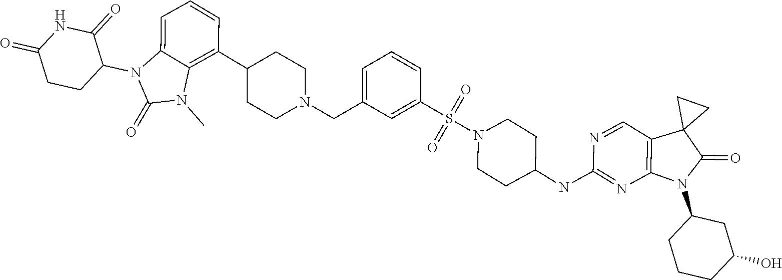 [SMILES: Cn1c(=O)n(C2CCC(=O)NC2=O)c2cccc(C3CCN(Cc4cccc(S(=O)(=O)N5CCC(Nc6ncc7c(n6)N([C@@H]6CCC[C@@H](O)C6)C(=O)C76CC6)CC5)c4)CC3)c21] | 852.5 | 1H NMR (400 MHz, DMSO-d6) δ 11.45-10.80 (m, 1H), 7.94-7.46 (m, 5H), 7.22-6.73 (m, 4H), 5.36 (dd, J = 12.6, 5.4 Hz, 1H), 4.66-4.47 (m, 1H), 4.03 (s, 1H), 3.65 (s, 2H), 3.58 (d, J = 12.8 Hz, 6H), 3.27-3.15 (m, 1H), 3.05-2.81 (m, 3H), 2.79-2.55 (m, 3H), 2.48-2.30 (m, 3H), 2.27-2.05 (m, 3H), 2.05-1.86 (m, 3H), 1.85-1.66 (m, 5H), 1.65-1.42 (m, 9H), 1.42- 1.35 (m, 2H). |
| 149 | 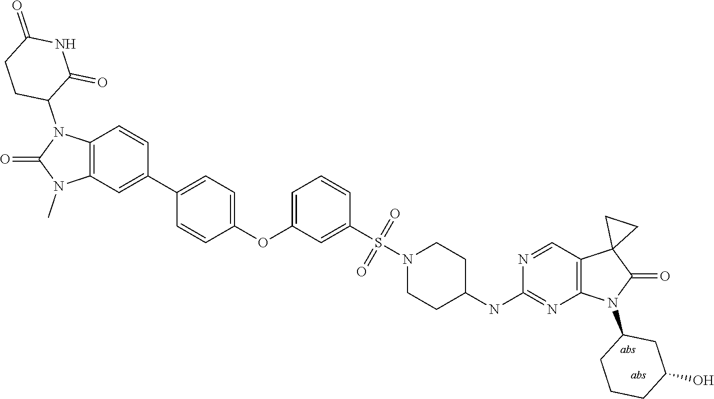 [SMILES: Cn1c(=O)n(C2CCC(=O)NC2=O)c2ccc(-c3ccc(Oc4cccc(S(=O)(=O)N5CCC(Nc6ncc7c(n6)N([C@@H]6CCC[C@@H](O)C6)C(=O)C76CC6)CC5)c4)cc3)cc21] | 847.3 | 1H NMR (300 MHz, DMSO-d6) δ 11.14 (s, 1H), 7.83-7.74 (m, 2H), 7.57-7.47 (m, 2H), 7.74- 7.62 (m, 2H), 7.42 (ddd, J = 8.1, 2.7, 0.9 Hz, 1H), 7.35 (dd, J = 8.2, 1.7 Hz, 1H), 7.31-7.17 (m, 4H), 7.11 (s, 1H), 5.42 (dd, J = 12.5, 5.4 Hz, 1H), 4.71-4.46 (m, 2H), 4.07 (s, 1H), 3.68 (s, 1H), 3.55 (d, J = 11.7 Hz, 2H), 3.42 (s, 3H), 3.01- 2.80 (m, 1H), 2.80-2.58 (m, 2H), 2.56 (s, 1H), 2.44 (d, J = 13.8 Hz, 1H), 2.25-2.00 (m, 2H), 1.93 (d, J = 11.8 Hz, 2H), 1.80-1.47 (m, 9H), 1.41 (q, J = 4.2, 3.6 Hz, 2H), 1.24 (s, 1H). |
| 150 | 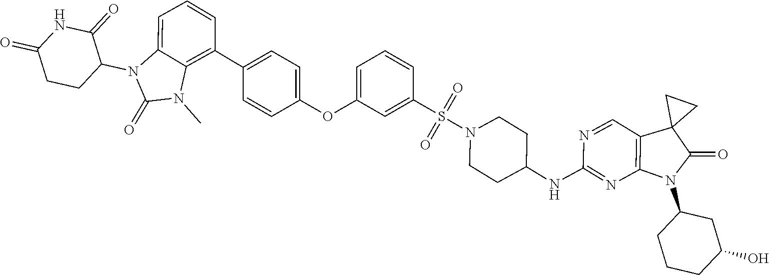 [SMILES: Cn1c(=O)n(C2CCC(=O)NC2=O)c2cccc(-c3ccc(Oc4cccc(S(=O)(=O)N5CCC(Nc6ncc7c(n6)N([C@@H]6CCC[C@@H](O)C6)C(=O)C76CC6)CC5)c4)cc3)c21] | 847.2 | 1H NMR (300 MHz, DMSO-d6) δ 11.04 (s, 1H), 7.81-7.60 (m, 2H), 7.59-7.40 (m, 4H), 7.35- 7.06 (m, 6H), 6.97-6.84 (m, 1H), 5.45 (dd, J = 12.4, 5.4 Hz, 1H), 4.69-4.49 (m, 2H), 4.06 (s, 1H), 3.69 (s, 1H), 3.54 (d, J = 11.8 Hz, 2H), 2.91 (s, 4H), 2.83-2.56 (m, 4H), 2.42 (d, J = 12.9 Hz, 1H), 2.24-2.01 (m, 2H), 1.90 (s, 2H), 1.78- 1.45 (m, 9H), 1.41 (t, J = 3.4 Hz, 2H), 1.24 (s, 1H) |
| 151 | 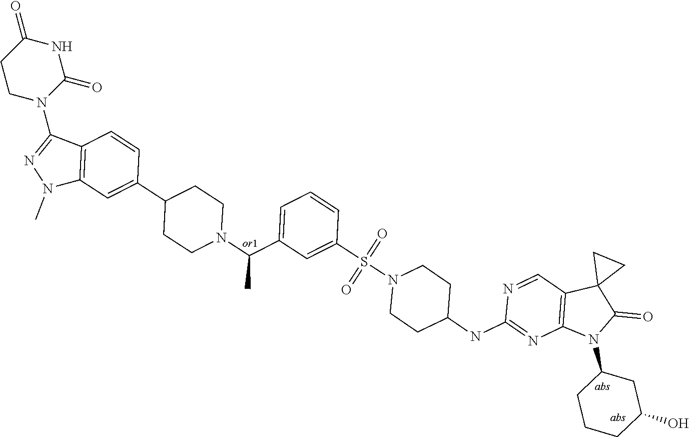 [SMILES: C[C@H](c1cccc(S(=O)(=O)N2CCC(Nc3ncc4c(n3)N([C@@H]3CCC[C@@H](O)C3)C(=O)C43CC3)CC2)c1)N1CCC(c2ccc3c(N4CCC(=O)NC4=O)nn(C)c3c2)CC1] | 851.4 | 1H NMR (400 MHz, DMSO-d6) δ 10.53 (s, 1H), 7.71 (d, J = 9.9 Hz, 2H), 7.63 (d, J = 4.6 Hz, 3H), 7.51 (d, J = 8.4 Hz, 1H), 7.40 (s, 1H), 7.12- 6.91 (m, 2H), 4.54 (d, J = 24.3 Hz, 2H), 4.11- 3.81 (m, 6H), 3.63 (dd, J = 35.9, 9.1 Hz, 4H), 3.10 (d, J = 10.8 Hz, 1H), 2.86 (d, J = 10.8 Hz, 1H), 2.75 (t, J = 6.6 Hz, 2H), 2.57 (d, J = 11.2 Hz, 1H), 2.42 (s, 3H), 2.07 (dt, J = 26.1, 10.0 Hz, 3H), 1.90 (s, 2H), 1.85-1.64 (m, 5H), 1.63- 1.26 (m, 13H). |
| 152 | 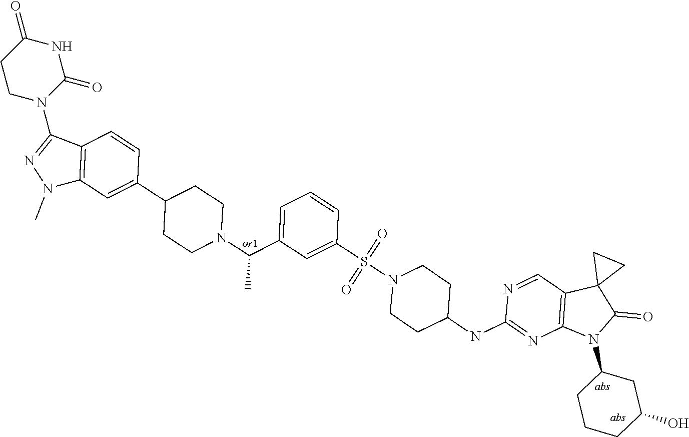 [SMILES: C[C@@H](c1cccc(S(=O)(=O)N2CCC(Nc3ncc4c(n3)N([C@@H]3CCC[C@@H](O)C3)C(=O)C43CC3)CC2)c1)N1CCC(c2ccc3c(N4CCC(=O)NC4=O)nn(C)c3c2)CC1] | 851.4 | 1H NMR (400 MHz, DMSO-d6) δ 10.52 (s, 1H), 7.71 (d, J = 6.3 Hz, 2H), 7.63 (d, J = 4.7 Hz, 3H), 7.51 (d, J = 8.4 Hz, 1H), 7.40 (s, 1H), 7.05 (s, 1H), 6.98 (d, J = 8.5 Hz, 1H), 4.65-4.47 (m, 2H), 4.03 (s, 1H), 3.97-3.83 (m, 5H), 3.67 (d, J = 6.7 Hz, 1H), 3.58 (d, J = 11.3 Hz, 3H), 3.11 (d, J = 10.7 Hz, 1H), 2.86 (d, J = 10.8 Hz, 1H), 2.75 (t, J = 6.6 Hz, 2H), 2.56 (d, J = 11.6 Hz, 1H), 2.43 (t, J = 10.9 Hz, 3H), 2.21-1.98 (m, 3H), 1.92 (s, 2H), 1.80 (d, J = 13.5 Hz, 2H), 1.77- 1.65 (m, 3H), 1.50 (dd, J = 49.7, 10.0 Hz, 8H), 1.37 (d, J = 6.2 Hz, 5H). |
| 153 | 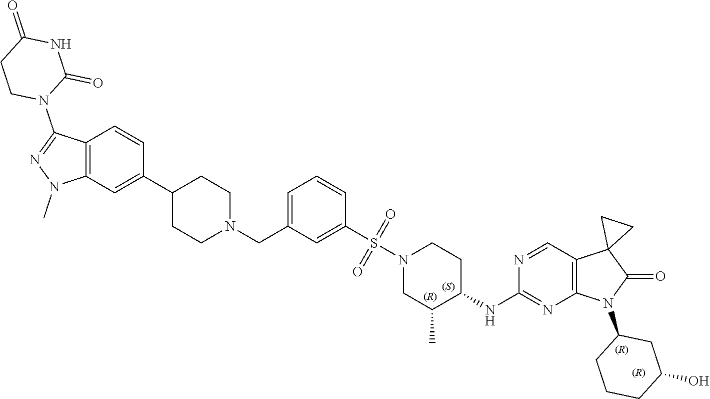 | 851.4 | 1H NMR (400 MHz, DMSO-d6) δ 10.53 (s, 1H), 7.73 (s, 1H), 7.71-7.59 (m, 4H), 7.52 (d, J = 8.4 Hz, 1H), 7.43 (s, 1H), 7.09 (s, 1H), 7.01 (d, J = 8.5 Hz, 1H), 4.59 (s, 1H), 4.50 (d, J = 2.7 Hz, 1H), 4.02 (s, 1H), 3.95 (s, 3H), 3.90 (t, J = 6.7 Hz, 3H), 3.65 (s, 2H), 3.18 (d, J = 33.4 Hz, 2H), 2.96 (d, J = 10.7 Hz, 2H), 2.87-2.71 (m, 4H), 2.66 (d, J = 6.9 Hz, 1H), 2.41 (t, J = 12.6 Hz, 1H), 2.15 (d, J = 14.2 Hz, 4H), 1.77 (d, J = 15.0 Hz, 5H), 1.73-1.53 (m, 5H), 1.47 (s, 3H), 1.36 (d, J = 4.2 Hz, 2H), 0.89 (d, J = 6.9 Hz, 3H). |
| 154 | 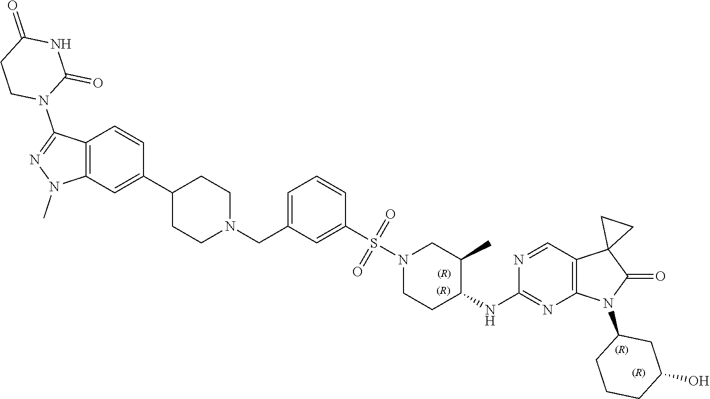 | 851.4 | 1H NMR (400 MHz, DMSO-d6) δ 10.53 (s, 1H), 7.80-7.49 (m, 6H), 7.43 (s, 1H), 7.02 (d, J = 8.6 Hz, 2H), 4.55 (d, J = 29.3 Hz, 2H), 3.92 (d, J = 17.7 Hz, 6H), 3.66 (q, J = 14.0 Hz, 4H), 3.54- 3.39 (m, 1H), 2.95 (t, J = 14.0 Hz, 2H), 2.74 (t, J = 6.6 Hz, 2H), 2.65 (dd, J = 14.3, 6.6 Hz, 1H), 2.43 (s, 1H), 2.30 (d, J = 18.7 Hz, 1H), 2.13 (dt, J = 20.8, 9.6 Hz, 3H), 2.02 (d, J = 11.3 Hz, 1H), 1.94 (d, J = 25.1 Hz, 1H), 1.79 (d, J = 9.9 Hz, 5H), 1.69 (d, J = 13.5 Hz, 1H), 1.48 (s, 7H), 1.40 1.31 (m, 3H), 0.84 (d, J = 6.4 Hz, 3H). |
| 155 | 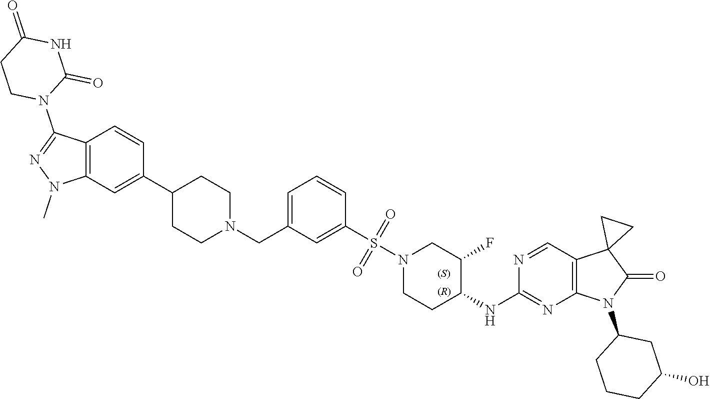 | 855.3 | 1H NMR (400 MHz, DMSO-d6) δ 10.54 (s, 1H), 7.76-7.67 (m, 2H), 7.69-7.58 (m, 3H), 7.54 (d, J = 8.4 Hz, 1H), 7.46 (s, 1H), 7.14 (s, 1H), 7.04 (d, J = 8.6 Hz, 1H), 4.90 (d, J = 49.0 Hz, 1H), 4.60 (s, 1H), 4.52 (d, J = 2.7 Hz, 1H), 4.05 (s, 1H), 3.96 (s, 3H), 3.90 (t, J = 6.7 Hz, 3H), 3.72 (d, J = 11.6 Hz, 1H), 3.64 (s, 2H), 2.96 (d, J = 10.7 Hz, 2H), 2.75 (t, J = 6.6 Hz, 2H), 2.72- 2.60 (m, 2H), 2.33 (t, J = 1.8 Hz, 1H), 2.25- 2.08 (m, 3H), 1.97-1.66 (m, 8H), 1.67-1.45 (m, 7H), 1.43-1.34 (m, 3H). |
| 156 | 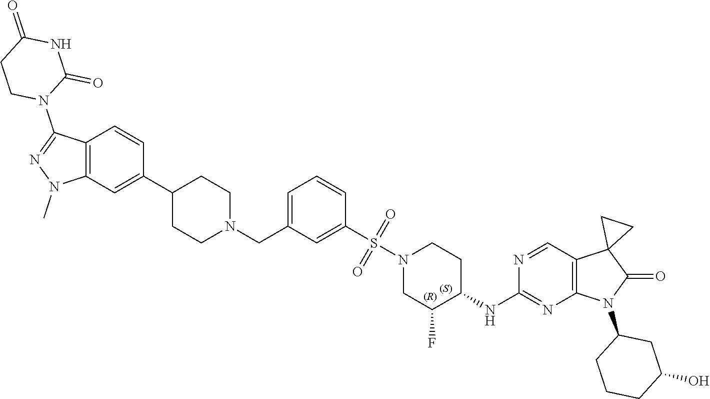 | 855.4 | 1H NMR (400 MHz, DMSO-d6) δ 10.54 (s, 1H), 7.79-7.58 (m, 5H), 7.54 (d, J = 8.5 Hz, 1H), 7.46 (s, 1H), 7.15 (s, 1H), 7.08-7.00 (m, 1H), 4.92 (d, J = 48.0 Hz, 1H), 4.60 (s, 1H), 4.52 (d, J = 2.7 Hz, 1H), 4.04 (s, 1H), 4.00-3.81 (m, 7H), 3.72 (d, J = 11.2 Hz, 1H), 3.64 (s, 2H), 3.28 (s, 2H), 2.96 (d, J = 10.7 Hz, 2H), 2.75 (t, J = 6.6 Hz, 2H), 2.67 (s, 2H), 2.14 (s, 3H), 1.96-1.67 (m, 7H), 1.68-1.45 (m, 6H), 1.41 (t, J = 3.5 Hz, 3H) |
| 157 | 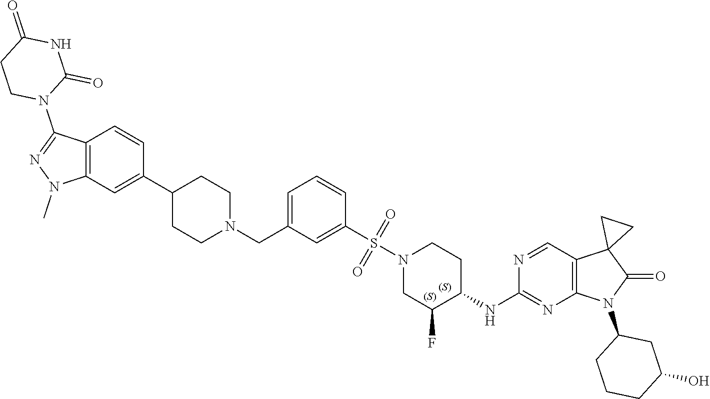 | 855.5 | 1H NMR (400 MHz, DMSO-d6) δ 10.54 (s, 1H), 7.80-7.60 (m, 5H), 7.52 (d, J = 8.5 Hz, 1H), 7.43 (s, 1H), 7.27 (d, J = 7.4 Hz, 1H), 7.00 (d, J = 8.5 Hz, 1H), 4.83-4.53 (m, 2H), 4.51 (d, J = 2.7 Hz, 1H), 4.05 (s, 1H), 3.98-3.84 (m, 5H), 3.65 (s, 2H), 3.46-3.42 (m, 2H)2.95 (d, J = 10.7 Hz, 3H), 2.75 (t, J = 6.7 Hz, 3H), 2.70-2.59 (m, 1H), 2.47-2.37 (m, 1H), 2.11 (d, J = 13.7 Hz, 3H), 2.00 (s, 1H), 1.87-1.54 (m, 9H), 1.45 (d, J = 30.2 Hz, 4H), 1.40-1.25 (m, 3H). |
| 158 | 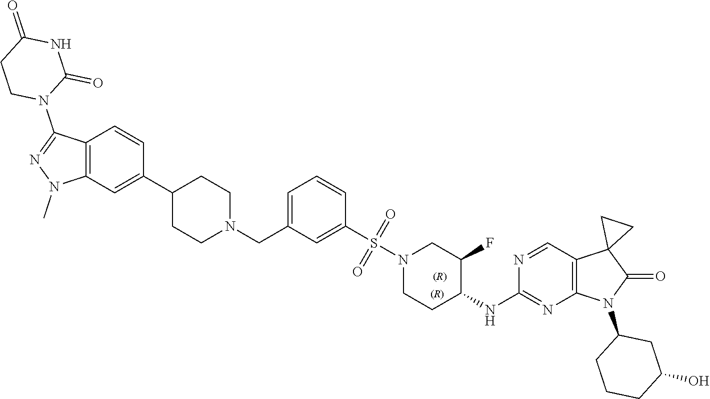 | 855.3 | 1H NMR (400 MHz, DMSO-d6) δ 10.54 (s, 1H), 7.83-7.59 (m, 5H), 7.52 (d, J = 8.4 Hz, 1H), 7.43 (s, 1H), 7.27 (d, J = 7.3 Hz, 1H), 7.01 (dd, J = 8.6, 1.3 Hz, 1H), 4.88-4.40 (m, 3H), 4.23- 3.82 (m, 7H), 3.65 (s, 3H), 2.95 (d, J = 10.8 Hz, 4H), 2.75 (t, J = 6.7 Hz, 2H), 2.69-2.57 (m, 1H), 2.47-2.35 (m, 1H), 2.24-2.08 (m, 3H), 2.01 (s, 1H), 1.85-1.44 (m, 12H), 1.38 (q, J = 3.6, 3.2 Hz, 2H), 1.30 (s, 1H). |
| 159 | 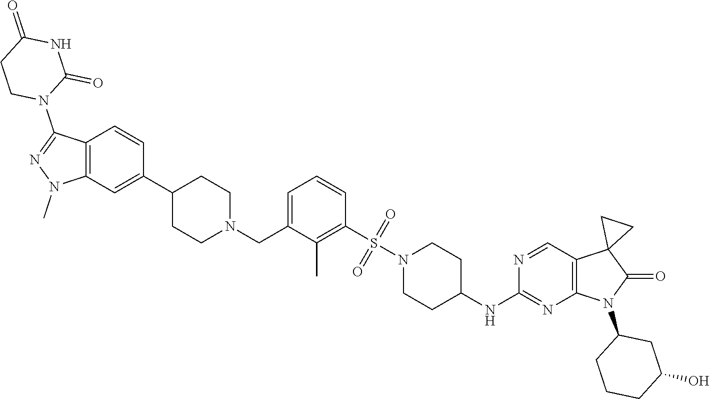 [SMILES: Cc1c(CN2CCC(c3ccc4c(N5CCC(=O)NC5=O)nn(C)c4c3)CC2)cccc1S(=O)(=O)N1CCC(Nc2ncc3c(n2)N([C@@H]2CCC[C@@H](O)C2)C(=O)C32CC2)CC1] | 851.3 | 1H NMR (400 MHz, DMSO-d6) δ 10.54 (s, 1H), 7.78 (d, J = 7.9 Hz, 1H), 7.71 (s, 1H), 7.63 (d, J = 7.5 Hz, 1H), 7.54 (d, J = 8.4 Hz, 1H), 7.45 (s, 1H), 7.39 (t, J = 7.8 Hz, 1H), 7.10 (d, J = 7.1 Hz, 1H), 7.04 (d, J = 8.5 Hz, 1H), 4.70-4.56 (m, 1H), 4.53 (s, 1H), 4.07 (s, 1H), 3.96 (s, 3H), 3.92 (s, 2H), 3.78 (s, 1H), 3.67-3.54 (m, 4H), 2.93 (d, J = 10.8 Hz, 2H), 2.84 (t, J = 11.5 Hz, 2H), 2.75 (t, J = 6.7 Hz, 2H), 2.63 (s, 4H), 2.45 (d, J = 13.6 Hz, 1H), 2.25-2.11 (m, 3H), 1.94 (s, 2H), 1.79 (d, J = 10.9 Hz, 5H), 1.75-1.44 (m, 8H), 1.41 (q, J = 4.1, 3.6 Hz, 2H), 1.33 (s, 1H). |
| 160 | 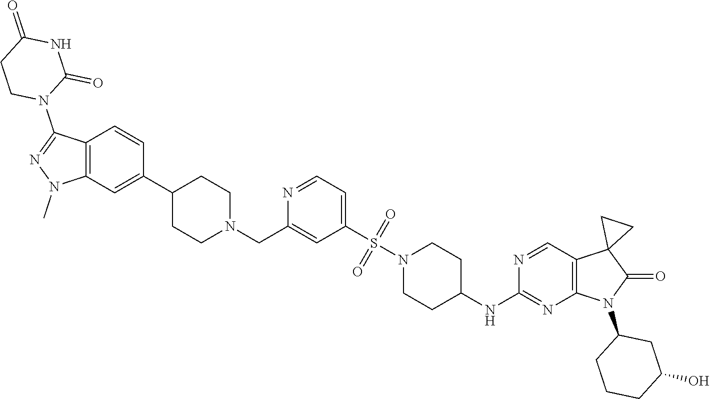 [SMILES: Cn1nc(N2CCC(=O)NC2=O)c2ccc(C3CCN(Cc4cc(S(=O)(=O)N5CCC(Nc6ncc7c(n6)N([C@@H]6CCC[C@@H](O)C6)C(=O)C76CC6)CC5)ccn4)CC3)cc21] | 838.3 | 1H NMR (400 MHz, DMSO-d6) δ 10.53 (s, 1H), 8.83 (d, J = 5.1 Hz, 1H), 7.77 (d, J = 1.8 Hz, 1H), 7.70-7.57 (m, 2H), 7.52 (d, J = 8.4 Hz, 1H), 7.44 (s, 1H), 7.03 (dd, J = 18.2, 7.8 Hz, 2H), 4.58 (s, 1H), 4.50 (d, J = 2.7 Hz, 1H), 4.04 (s, 1H), 3.95 (s, 3H), 3.90 (t, J = 6.7 Hz, 2H), 3.80 (s, 2H), 3.69 (s, 1H), 3.58 (d, J = 11.4 Hz, 2H), 2.98 (d, J = 10.8 Hz, 2H), 2.75 (t, J = 6.6 Hz, 2H), 2.65 (t, J = 10.1 Hz, 3H), 2.42 (t, J = 12.6 Hz, 1H), 2.22 (s, 2H), 2.11 (dd, J = 19.0, 8.6 Hz, 1H), 1.90 (d, J = 11.4 Hz, 2H), 1.80 (s, 4H), 1.72-1.41 (m, 10H), 1.37 (q, J = 4.1, 3.6 Hz, 2H). |
| 161 | 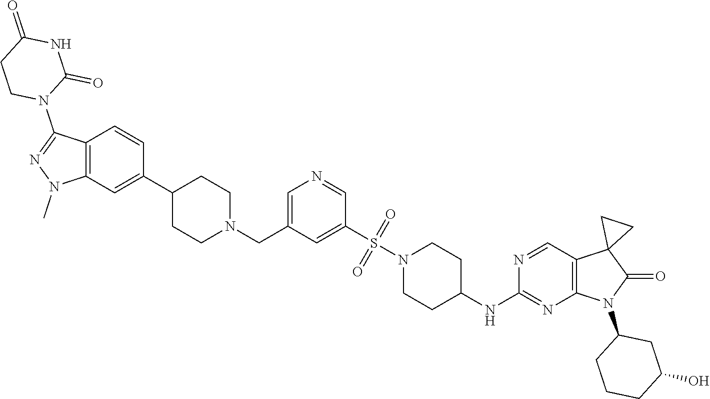 [SMILES: Cn1nc(N2CCC(=O)NC2=O)c2ccc(C3CCN(Cc4cncc(S(=O)(=O)N5CCC(Nc6ncc7c(n6)N([C@@H]6CCC[C@@H](O)C6)C(=O)C76CC6)CC5)c4)CC3)cc21] | 838.4 | 1H NMR (400 MHz, DMSO-d6) δ 10.55 (s, 1H), 8.84 (dd, J = 15.1, 2.0 Hz, 2H), 8.09 (s, 1H), 7.65 (s, 1H), 7.53 (d, J = 8.4 Hz, 1H), 7.44 (s, 1H), 7.08 (s, 1H), 7.02 (d, J = 8.5 Hz, 1H), 4.58 (t, J = 11.7 Hz, 2H), 4.53 (d, J = 2.7 Hz, 2H), 4.04 (s, 1H), 3.95 (s, 3H), 3.90 (t, J = 6.6 Hz, 2H), 3.71 (s, 3H), 3.60 (d, J = 11.5 Hz, 2H), 2.95 (d, J = 10.7 Hz, 2H), 2.75 (t, J = 6.7 Hz, 2H), 2.72-2.53 (m, 3H), 2.42 (t, J = 12.5 Hz, 1H), 2.26-2.06 (m, 3H), 1.99-1.85 (m, 2H), 1.83- 1.73 (m, 4H), 1.73-1.42 (m, 10H), 1.43-1.29 (m, 2H). |
| 162 | 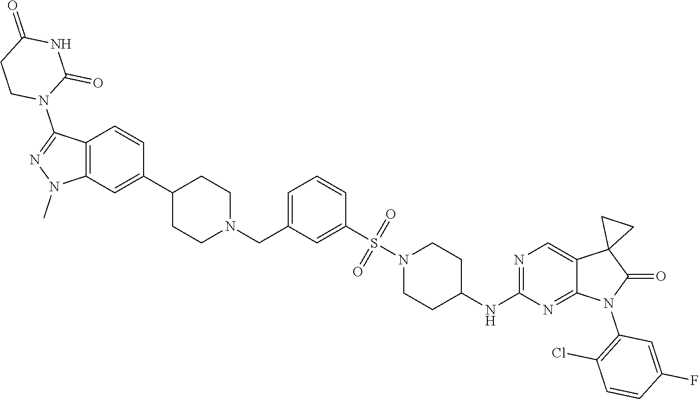 [SMILES: Cn1nc(N2CCC(=O)NC2=O)c2ccc(C3CCN(Cc4cccc(S(=O)(=O)N5CCC(Nc6ncc7c(n6)N(c6cc(F)ccc6Cl)C(=O)C76CC6)CC5)c4)CC3)cc21] | 867.3 | 1H NMR (400 MHz, DMSO-d6) δ 10.54 (s, 1H), 7.79 (s, 1H), 7.75-7.65 (m, 3H), 7.65-7.56 (m, 3H), 7.56-7.52 (m, 1H), 7.43 (d, J = 10.6 Hz, 2H), 7.16 (d, J = 7.4 Hz, 1H), 7.03 (dd, J = 8.5, 1.2 Hz, 1H), 3.95 (s, 3H), 3.90 (t, J = 6.7 Hz, 2H), 3.64 (s, 2H), 3.50 (s, 2H), 2.94 (d, J = 10.5 Hz, 2H), 2.75 (t, J = 6.7 Hz, 2H), 2.72-2.59 (m, 1H), 2.44 (t, J = 11.3 Hz, 2H), 2.20-2.08 (m, 2H), 1.90-1.71 (m, 7H), 1.64 (s, 2H), 1.59- 1.40 (m, 4H). |
| 163 | 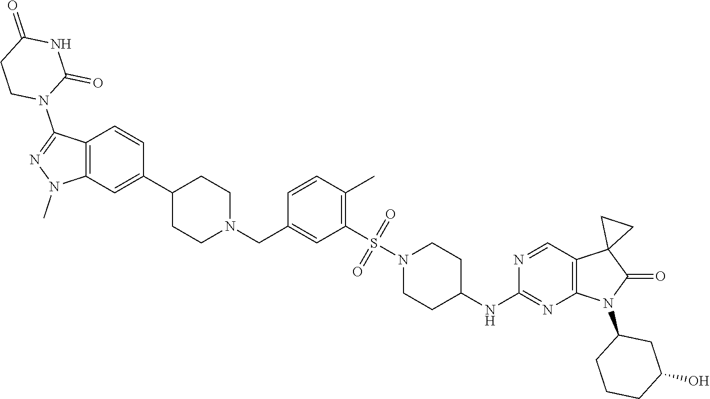 [SMILES: Cc1ccc(CN2CCC(c3ccc4c(N5CCC(=O)NC5=O)nn(C)c4c3)CC2)cc1S(=O)(=O)N1CCC(Nc2ncc3c(n2)N([C@@H]2CCC[C@@H](O)C2)C(=O)C32CC2)CC1] | 851.3 | 1H NMR (400 MHz, DMSO-d6) δ 10.53 (s, 1H), 7.79 (s, 1H), 7.67 (s, 1H), 7.53 (d, J = 8.1 Hz, 2H), 7.46-7.36 (m, 2H), 7.11-7.05 (m, 1H), 7.02 (d, J = 8.5 Hz, 1H), 4.60 (t, J = 9.3 Hz, 1H), 4.52 (d, J = 2.7 Hz, 1H), 4.06 (s, 1H), 3.95 (s, 3H), 3.90 (t, J = 6.7 Hz, 2H), 3.65-3.52 (m, 4H), 2.93 (d, J = 10.8 Hz, 2H), 2.82-2.70 (m, 4H), 2.70-2.54 (m, 4H), 2.47-2.37 (m, 1H), 2.24-2.03 (m, 4H), 2.00-1.85 (m, 2H), 1.84- 1.70 (m, 5H), 1.70-1.44 (m, 9H), 1.42-1.32 (m, 2H). |
| 164 | 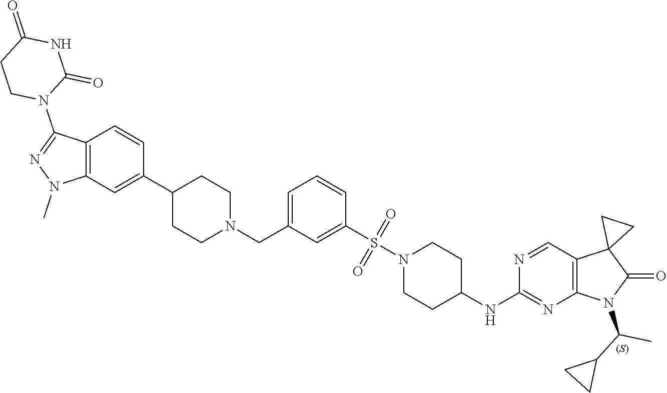 | 807.4 | 1H NMR (400 MHz, DMSO-d6) δ 10.54 (s, 1H), 7.77-7.59 (m, 5H), 7.53 (d, J = 8.5 Hz, 1H), 7.44 (s, 1H), 7.11-6.90 (m, 2H), 3.95 (s, 3H), 3.90 (t, J = 6.7 Hz, 2H), 3.65 (s, 3H), 3.54 (d, J = 10.9 Hz, 2H), 3.46 (dt, J = 10.0, 7.0 Hz, 1H), 2.95 (d, J = 10.7 Hz, 2H), 2.75 (t, J = 6.7 Hz, 2H), 2.66 (h, J = 8.5 Hz, 1H), 2.47 (s, 1H), 2.12 (dt, J = 16.0, 9.8 Hz, 2H), 1.91 (d, J = 12.6 Hz, 2H), 1.85-1.72 (m, 4H), 1.70-1.41 (m, 9H), 1.40 (q, J = 3.3 Hz, 2H), 0.53 (s, 1H), 0.38- 0.20 (m, 2H), 0.09-0.01 (m, 1H). |
| 165 | 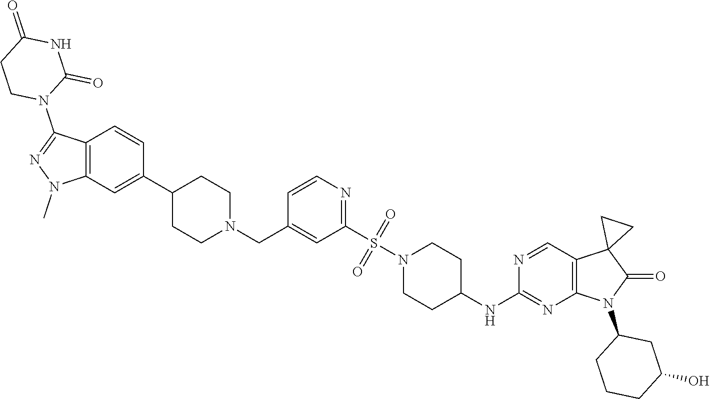 [SMILES: Cn1nc(N2CCC(=O)NC2=O)c2ccc(C3CCN(Cc4ccnc(S(=O)(=O)N5CCC(Nc6ncc7c(n6)N([C@@H]6CCC[C@@H](O)C6)C(=O)C76CC6)CC5)c4)CC3)cc21] | 838.3 | 1H NMR (400 MHz, DMSO-d6) δ 10.53 (s, 1H), 8.71 (d, J = 4.9 Hz, 1H), 7.91 (s, 1H), 7.69 (d, J = 5.8 Hz, 2H), 7.55 (d, J = 8.5 Hz, 1H), 7.47 (s, 1H), 7.20-6.79 (m, 2H), 4.60 (t, J = 11.6 Hz, 1H), 4.52 (d, J = 2.6 Hz, 1H), 4.05 (s, 1H), 3.97 (s, 3H), 3.90 (t, J = 6.6 Hz, 2H), 3.79-3.65 (m, 5H), 2.94 (d, J = 10.8 Hz, 2H), 2.84 (t, J = 11.7 Hz, 2H), 2.75 (t, J = 6.6 Hz, 2H), 2.71-2.61 (m, 3H), 2.47-2.39 (m, 1H), 2.25-2.09 (m, 3H), 1.96-1.86 (m, 2H), 1.86-1.75 (m, 3H), 1.77- 1.55 (m, 4H), 1.56-1.46 (m, 5H), 1.43-1.34 (m, 2H). |
| 166 | 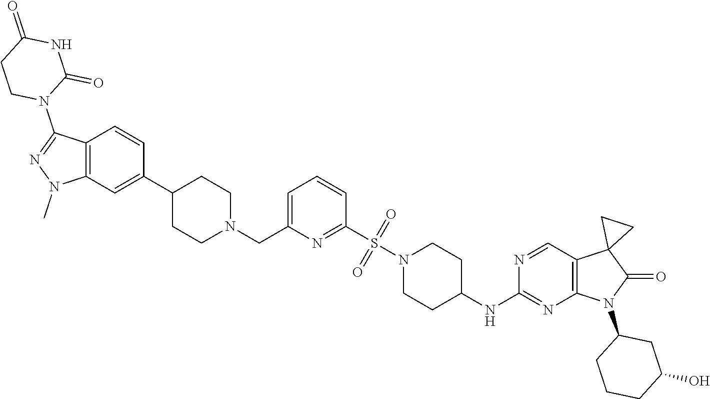 [SMILES: Cn1nc(N2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)n4)CC3)cc21] | 838.3 | 1H NMR (400 MHz, DMSO-d6) δ 10.55 (s, 1H), 8.11 (t, J = 7.8 Hz, 1H), 7.81 (t, J = 8.2 Hz, 2H), 7.72-7.56 (m, 1H), 7.53 (d, J = 8.5 Hz, 1H), 7.44 (s, 1H), 7.09 (s, 1H), 7.03 (dd, J = 8.6, 1.3 Hz, 1H), 4.69-4.46 (m, 2H), 4.07 (s, 1H), 3.99-3.85 (m, 5H), 3.80-3.62 (m, 5H), 2.98 (d, J = 10.7 Hz, 2H), 2.87 (t, J = 11.9 Hz, 2H), 2.75 (t, J = 6.7 Hz, 2H), 2.70-2.60 (m, 1H), 2.49-2.39 (m, 1H), 2.30-2.09 (m, 3H), 1.94 (s, 2H), 1.88-1.77 (m, 4H), 1.76-1.54 (m, 3H), 1.55-1.43 (m, 6H), 1.43-1.28 (m, 3H). |
| 167 | 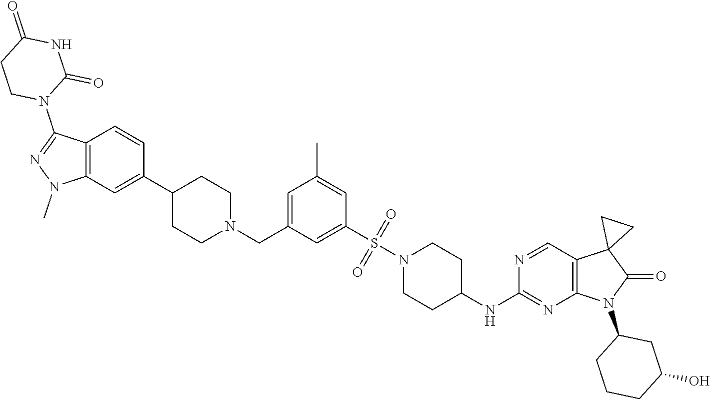 [SMILES: Cc1cc(CN2CCC(c3ccc4c(N5CCC(=O)NC5=O)nn(C)c4c3)CC2)cc(S(=O)(=O)N2CCC(Nc3ncc4c(n3)N([C@@H]3CCC[C@@H](O)C3)C(=O)C43CC3)CC2)c1] | 851.4 | 1H NMR (400 MHz, DMSO-d6) δ 10.48 (s, 1H), 7.64 (s, 1H), 7.52 (t, J = 8.1 Hz, 3H), 7.45 (d, J = 5.2 Hz, 2H), 7.16-6.74 (m, 2H), 4.55 (d, J = 23.9 Hz, 2H), 4.11-3.84 (m, 6H), 3.58 (d, J = 10.0 Hz, 5H), 2.94 (d, J = 10.7 Hz, 2H), 2.81- 2.57 (m, 3H), 2.43 (s, 6H), 2.13 (d, J = 12.9 Hz, 3H), 1.92 (s, 2H), 1.78 (s, 5H), 1.65-1.32 (m, 10H). |
| 168 | 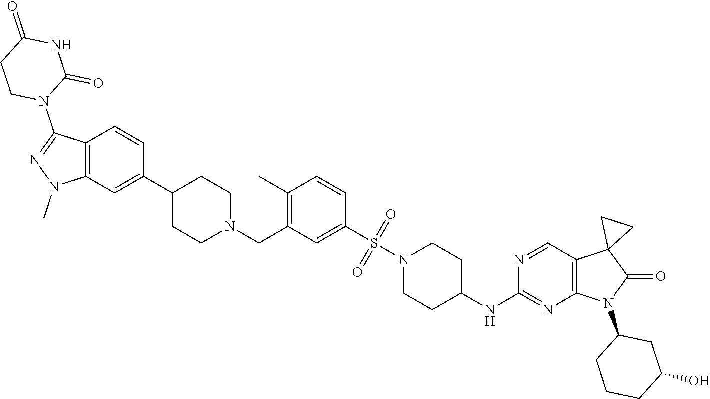 [SMILES: Cc1ccc(S(=O)(=O)N2CCC(Nc3ncc4c(n3)N([C@@H]3CCC[C@@H](O)C3)C(=O)C43CC3)CC2)cc1CN1CCC(c2ccc3c(N4CCC(=O)NC4=O)nn(C)c3c2)CC1] | 851.3 | 1H NMR (400 MHz, DMSO-d6) δ 10.55 (s, 1H), 7.69 (s, 1H), 7.63 (s, 1H), 7.58-7.49 (m, 2H), 7.48-7.34 (m, 2H), 7.12-6.95 (m, 2H), 4.65- 4.41 (m, 2H), 4.03 (s, 1H), 3.95 (s, 3H), 3.90 (t, J = 6.7 Hz, 2H), 3.65-3.44 (m, 5H), 2.94 (d, J = 10.6 Hz, 2H), 2.75 (t, J = 6.6 Hz, 2H), 2.70- 2.60 (m, 1H), 2.47-2.30 (m, 6H), 2.26-2.08 (m, 3H), 1.97-1.83 (m, 2H), 1.86-1.64 (m, 5H), 1.63-1.30 (m, 11H). |
| 169 | 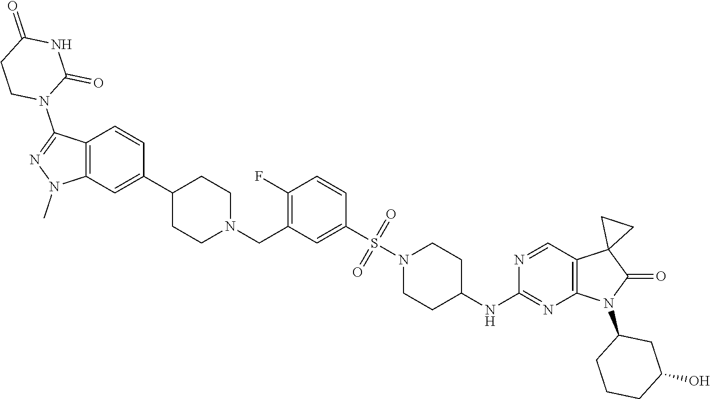 [SMILES: Cn1nc(N2CCC(=O)NC2=O)c2ccc(C3CCN(Cc4cc(S(=O)(=O)N5CCC(Nc6ncc7c(n6)N([C@@H]6CCC[C@@H](O)C6)C(=O)C76CC6)CC5)ccc4F)CC3)cc21] | 855.5 | 1H NMR (400 MHz, DMSO-d6) δ 10.53 (s, 1H), 7.87 (dd, J = 6.8, 2.4 Hz, 1H), 7.80-7.68 (m, 1H), 7.63 (s, 1H), 7.54-7.39 (m, 3H), 7.06 (s, 1H), 7.00 (d, J = 8.5 Hz, 1H), 4.58 (s, 1H), 4.51 (d, J = 2.7 Hz, 1H), 4.04 (s, 1H), 3.94 (s, 3H), 3.90 (t, J = 6.7 Hz, 2H), 3.68 (s, 3H), 3.55 (d, J = 11.4 Hz, 2H), 2.97 (d, J = 10.6 Hz, 2H), 2.75 (t, J = 6.6 Hz, 2H), 2.64 (td, J = 10.3, 9.0, 3.4 Hz, 1H), 2.58-2.52 (m, 2H), 2.41 (t, J = 12.7 Hz, 1H), 2.23-2.05 (m, 3H), 1.90 (d, J = 11.9 Hz, 2H), 1.84-1.62 (m, 6H), 1.61-1.41 (m, 8H), 1.37 (q, J = 4.1, 3.6 Hz, 2H). |
| 170 | 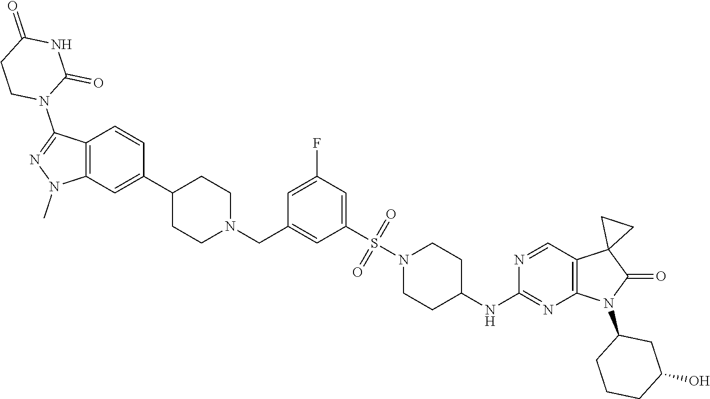 [SMILES: Cn1nc(N2CCC(=O)NC2=O)c2ccc(C3CCN(Cc4cc(F)cc(S(=O)(=O)N5CCC(Nc6ncc7c(n6)N([C@@H]6CCC[C@@H](O)C6)C(=O)C76CC6)CC5)c4)CC3)cc21] | 855.3 | 1H NMR (400 MHz, DMSO-d6) δ 10.54 (s, 1H), 7.80-7.32 (m, 6H), 7.25-6.89 (m, 2H), 4.58 (s, 1H), 4.52 (d, J = 2.7 Hz, 1H), 4.04 (s, 1H), 3.95 (s, 3H), 3.90 (t, J = 6.7 Hz, 2H), 3.67 (s, 3H), 3.58 (d, J = 11.4 Hz, 2H), 2.95 (d, J = 10.8 Hz, 2H), 2.75 (t, J = 6.7 Hz, 2H), 2.64 (dd, J = 15.3, 7.5 Hz, 1H), 2.56 (d, J = 11.9 Hz, 1H), 2.42 (t, J = 12.8 Hz, 2H), 2.11 (d, J = 30.1 Hz, 3H), 1.90 (s, 2H), 1.79 (s, 4H), 1.73-1.41 (m, 9H), 1.37 (d, J = 3.8 Hz, 2H). |
| 171 | 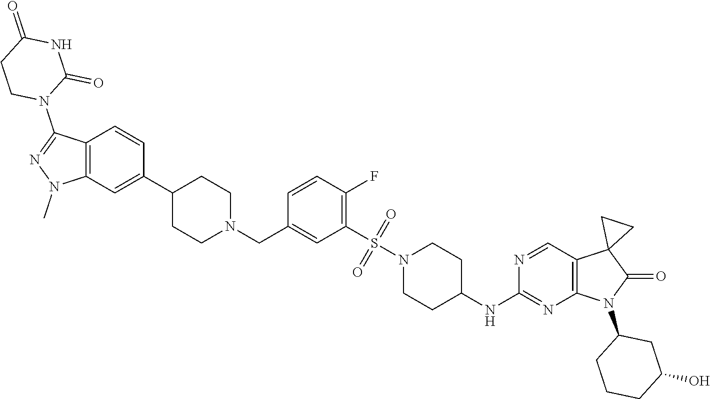 [SMILES: Cn1nc(N2CCC(=O)NC2=O)c2ccc(C3CCN(Cc4ccc(F)c(S(=O)(=O)N5CCC(Nc6ncc7c(n6)N([C@@H]6CCC[C@@H](O)C6)C(=O)C76CC6)CC5)c4)CC3)cc21] | 855.3 | 1H NMR (400 MHz, DMSO-d6) δ 10.54 (s, 1H), 7.80-7.61 (m, 3H), 7.53 (d, J = 8.5 Hz, 1H), 7.51-7.40 (m, 2H), 7.09 (s, 1H), 7.03 (d, J = 8.5 Hz, 1H), 4.58 (d, J = 12.9 Hz, 1H), 4.52 (d, J = 2.7 Hz, 1H), 4.05 (s, 1H), 3.96 (s, 3H), 3.90 (t, J = 6.6 Hz, 2H), 3.75 (d, J = 21.5 Hz, 1H), 3.69- 3.56 (m, 4H), 2.93 (d, J = 10.8 Hz, 2H), 2.80- 2.60 (m, 6H), 2.43 (d, J = 12.7 Hz, 1H), 2.24- 2.07 (m, 3H), 1.93 (s, 2H), 1.75 (d, J = 26.4 Hz, 13H), 1.39 (q, J = 4.1, 3.6 Hz, 2H). |
| 172 | 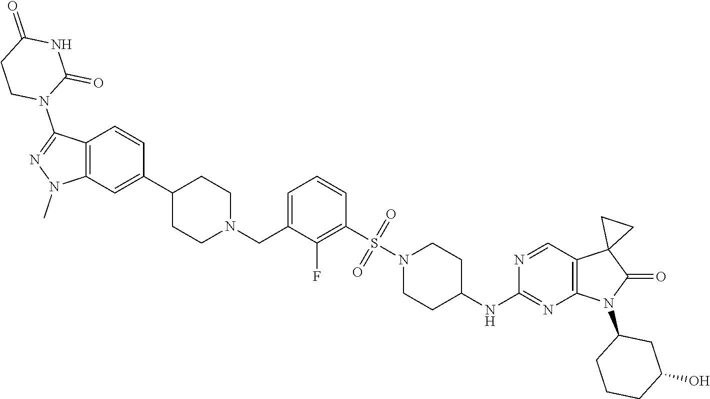 [SMILES: Cn1nc(N2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)c4F)CC3)cc21] | 855.4 | 1H NMR (400 MHz, DMSO-d6) δ 7.81 (t, J = 7.1 Hz, 1H), 7.71 (dd, J = 12.6, 5.6 Hz, 2H), 7.53 (d, J = 8.4 Hz, 1H), 7.48-7.38 (m, 2H), 7.10 (s, 1H), 7.03 (d, J = 8.5 Hz, 1H), 4.67-4.55 (m, 1H), 4.52 (d, J = 2.7 Hz, 1H), 4.05 (s, 1H), 3.95 (s, 3H), 3.90 (t, J = 6.7 Hz, 2H), 3.65 (d, J = 6.0 Hz, 5H), 2.96 (d, J = 10.6 Hz, 2H), 2.83-2.59 (m, 5H), 2.46-2.40 (m, 1H), 2.16 (d, J = 13.6 Hz, 3H), 1.94 (s, 2H), 1.85-1.67 (m, 5H), 1.67 -1.44 (m, 8H), 1.40 (q, J = 4.2, 3.6 Hz, 2H), 1.31 (s, 1H). |
| 174 | 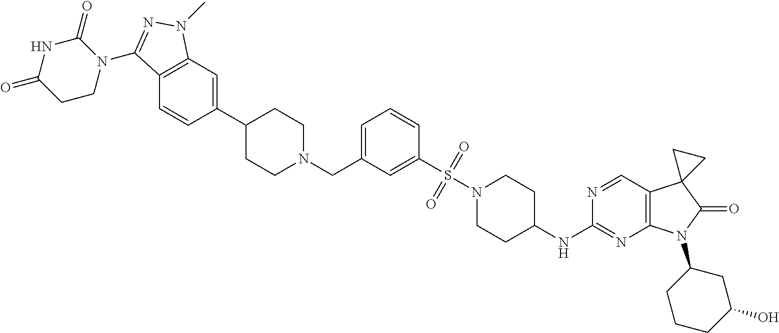 [SMILES: Cn1nc(N2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)c4)CC3)cc21] | 851.4 | 1H NMR (400 MHz, DMSO-d6) δ 10.54 (s, 1H), 8.05 (s, 1H), 7.79-7.59 (m, 4H), 7.53 (d, J = 8.4 Hz, 1H), 7.44 (s, 1H), 7.02 (d, J = 8.5 Hz, 1H), 6.37 (d, J = 7.3 Hz, 1H), 4.66 (s, 1H), 4.53 (d, J = 2.7 Hz, 1H), 4.36-4.18 (m, 2H), 4.04 (s, 1H), 4.00-3.82 (m, 5H), 3.65 (s, 2H), 3.55 (d, J = 11.8 Hz, 3H), 2.95 (d, J = 10.7 Hz, 2H), 2.74 (t, J = 6.7 Hz, 2H), 2.70-2.57 (m, 1H), 2.47-2.41 (m, 3H), 2.39-2.25 (m, 1H), 2.14 (s, 2H), 2.08 (s, 1H), 2.06-1.86 (m, 4H), 1.79 (s, 4H), 1.58 (tq, J = 27.8, 13.9, 12.4 Hz, 7H), 1.27 (d, J = 13.1 Hz, 1H). |
| 175 | 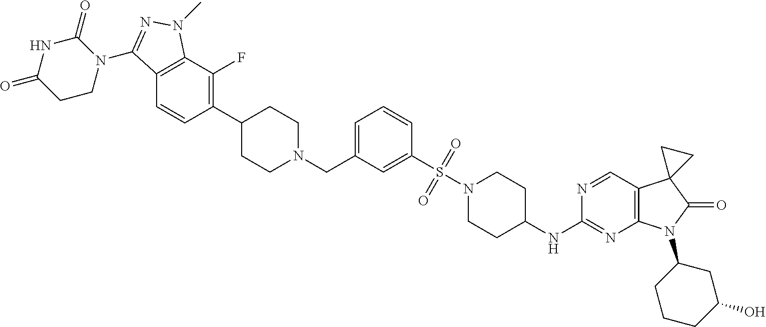 [SMILES: Cn1nc(N2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)c4)CC3)c(F)c21] | 855.3 | 1H NMR (400 MHz, DMSO-d6) δ 10.59 (s, 1H), 7.73 (s, 1H), 7.71-7.67 (m, 1H), 7.67-7.58 (m, 3H), 7.37 (d, J = 8.5 Hz, 1H), 7.05 (dd, J = 8.5, 5.5 Hz, 2H), 4.65-4.46 (m, 2H), 4.09 (d, J = 1.1 Hz, 4H), 3.90 (t, J = 6.7 Hz, 2H), 3.65 (s, 3H), 3.56 (d, J = 11.9 Hz, 3H), 2.94 (d, J = 11.3 Hz, 3H), 2.75 (t, J = 6.7 Hz, 2H), 2.41 (t, J = 10.3 Hz, 2H), 2.14 (q, J = 12.6, 12.0 Hz, 3H), 1.98-1.65 (m, 7H), 1.65-1.36 (m, 9H), 1.29 (d, J = 43.8 Hz, 2H). |
| 176 | 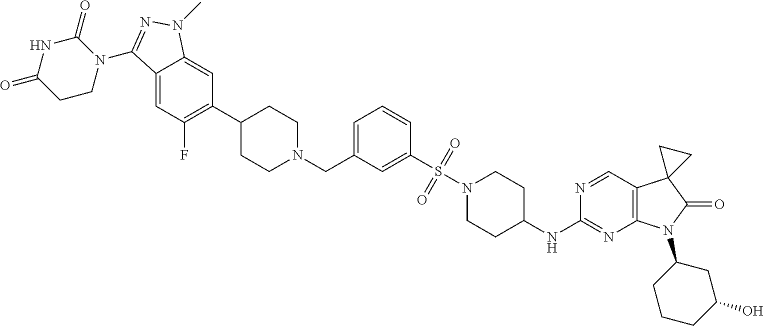 [SMILES: Cn1nc(N2CCC(=O)NC2=O)c2cc(F)c(C3CCN(Cc4cccc(S(=O)(=O)N5CCC(Nc6ncc7c(n6)N([C@@H]6CCC[C@@H](O)C6)C(=O)C76CC6)CC5)c4)CC3)cc21] | 855.4 | 1H NMR (400 MHz, DMSO-d6) δ 10.55 (s, 1H), 7.84-7.52 (m, 6H), 7.35 (d, J = 10.8 Hz, 1H), 7.05 (d, J = 7.1 Hz, 1H), 4.70-4.42 (m, 2H), 3.98 (s, 4H), 3.90 (t, J = 6.7 Hz, 2H), 3.76-3.47 (m, 5H), 3.07-2.82 (m, 3H), 2.75 (t, J = 6.7 Hz, 2H), 2.45 (d, J = 10.3 Hz, 3H), 2.13 (dt, J = 24.8, 13.2 Hz, 3H), 2.00-1.74 (m, 6H), 1.72-1.27 (m, 11H). |
| 177 | 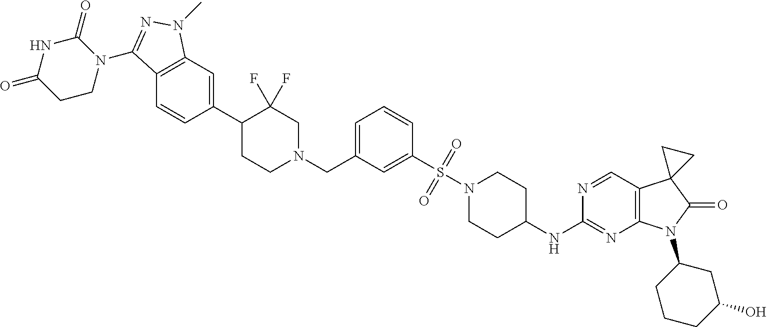 [SMILES: Cn1nc(N2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)c4)CC3(F)F)cc21] | 873.4 | 1H NMR (400 MHz, DMSO-d6) δ 10.56 (s, 1H), 7.81-7.61 (m, 5H), 7.60-7.44 (m, 2H), 7.07 (d, J = 8.5 Hz, 2H), 4.68-4.43 (m, 2H), 4.03 (s, 1H), 3.98 (s, 3H), 3.92 (t, J = 6.7 Hz, 2H), 3.87- 3.72 (m, 2H), 3.58 (d, J = 11.5 Hz, 3H), 3.30- 3.08 (m, 2H), 3.06-2.91 (m, 1H), 2.76 (t, J = 6.7 Hz, 2H), 2.58-2.51 (m, 1H), 2.47-2.37 (m, 3H), 2.39-2.22 (m, 2H), 2.19-2.04 (m, 1H), 1.97-1.82 (m, 3H), 1.73-1.42 (m, 9H), 1.43-1.33 (m, 2H), 1.34-1.26 (m, 1H). |
| 178 | 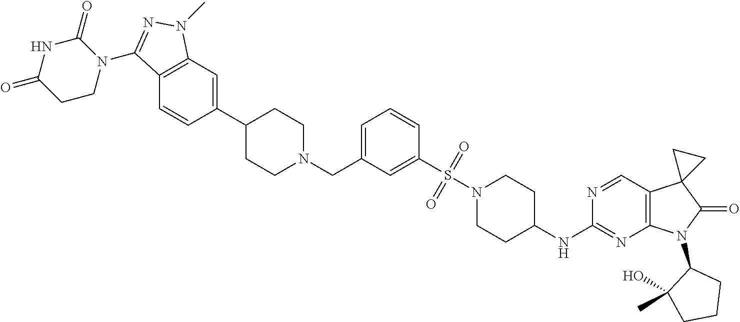 [SMILES: Cn1nc(N2CCC(=O)NC2=O)c2ccc(C3CCN(Cc4cccc(S(=O)(=O)N5CCC(Nc6ncc7c(n6)N([C@H]6CCC[C@]6(C)O)C(=O)C76CC6)CC5)c4)CC3)cc21] | 837.4 | 1H NMR (400 MHz, DMSO-d6) δ 10.54 (s, 1H), 7.78-7.59 (m, 5H), 7.53 (d, J = 8.5 Hz, 1H), 7.44 (s, 1H), 7.02 (dd, J = 8.5, 1.3 Hz, 1H), 4.52 (d, J = 11.8 Hz, 2H), 3.95 (s, 3H), 3.90 (t, J = 6.7 Hz, 2H), 3.65 (s, 2H), 3.57 (d, J = 11.5 Hz, 3H), 2.95 (d, J = 10.4 Hz, 2H), 2.75 (t, J = 6.7 Hz, 2H), 2.70-2.58 (m, 1H), 2.42 (t, J = 11.5 Hz, 2H), 2.34-2.20 (m, 1H), 2.14 (s, 2H), 1.92 (d, J = 12.4 Hz, 4H), 1.84-1.63 (m, 6H), 1.64- 1.45 (m, 5H), 1.39 (s, 2H). |
| 179 | 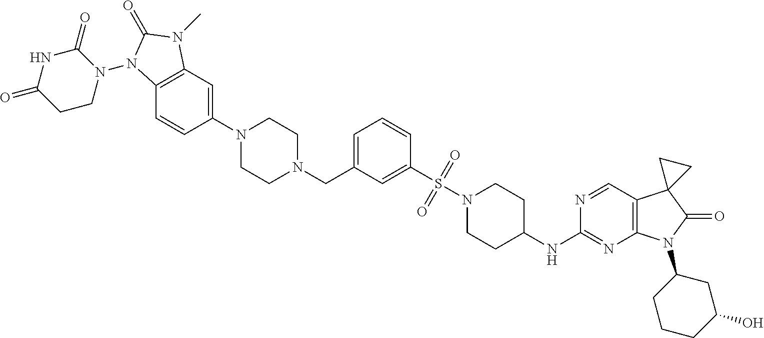 [SMILES: Cn1c(=O)n(N2CCC(=O)NC2=O)c2ccc(N3CCN(Cc4cccc(S(=O)(=O)N5CCC(Nc6ncc7c(n6)N([C@@H]6CCC[C@@H](O)C6)C(=O)C76CC6)CC5)c4)CC3)cc21] | 853.6 | 1H NMR (400 MHz, DMSO-d6) δ 11.05 (s, 1H), 7.84-7.54 (m, 5H), 7.07 (d, J = 7.0 Hz, 1H), 6.92 (d, J = 8.6 Hz, 1H), 6.83 (d, J = 2.2 Hz, 1H), 6.59 (dd, J = 8.6, 2.2 Hz, 1H), 5.28 (dd, J = 12.9, 5.4 Hz, 1H), 4.57 (d, J = 26.2 Hz, 2H), 4.04 (s, 1H), 3.74-3.51 (m, 5H), 3.29 (s, 3H), 3.18- 3.02 (m, 4H), 2.89 (ddd, J = 18.9, 15.0, 5.3 Hz, 1H), 2.74-2.54 (m, 6H), 2.44 (t, J = 12.0 Hz, 3H), 2.16 (q, J = 12.5 Hz, 1H), 2.03-1.85 (m, 3H), 1.78-1.45 (m, 10H), 1.40 (q, J = 4.0, 3.6 Hz, 2H). |
| 180 | 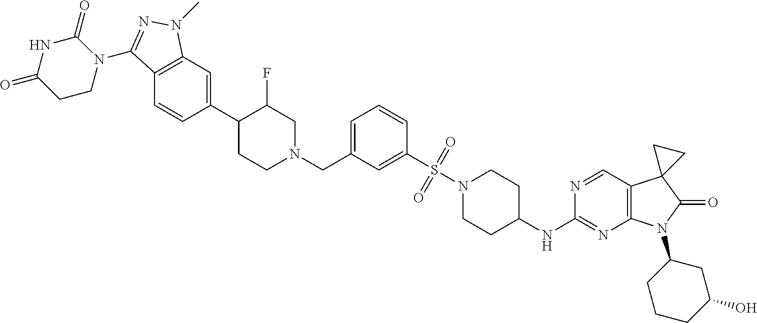 [SMILES: Cn1nc(N2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)c4)CC3F)cc21] | 855.4 | 1H NMR (400 MHz, DMSO-d6) δ 10.48 (s, 1H), 7.69 (s, 1H), 7.65-7.53 (m, 4H), 7.49 (dd, J = 8.5, 2.3 Hz, 1H), 7.39 (s, 1H), 7.02 (d, J = 8.6 Hz, 1H), 6.96 (s, 1H), 4.77 (d, J = 48.4 Hz, 1H), 4.50 (s, 2H), 3.97 (s, 1H), 3.89 (d, J = 1.9 Hz, 3H), 3.84 (t, J = 6.7 Hz, 2H), 3.71-3.56 (m, 5H), 3.09 (t, J = 11.5 Hz, 1H), 2.90 (d, J = 37.9 Hz, 2H), 2.69 (t, J = 6.6 Hz, 2H), 2.42- 2.30 (m, 4H), 2.30-2.15 (m, 2H), 2.05 (t, J = 12.5 Hz, 1H), 1.85 (s, 2H), 1.70-1.28 (m, 13H). |
| 181 | 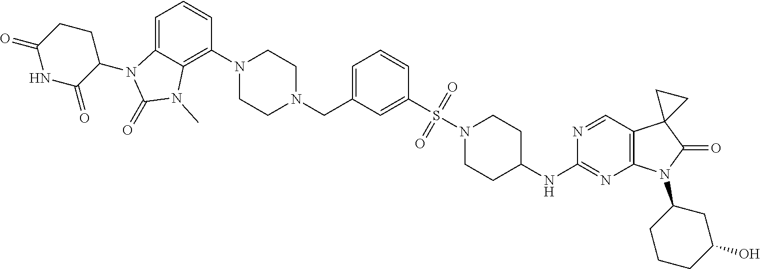 [SMILES: Cn1c(=O)n(C2CCC(=O)NC2=O)c2cccc(N3CCN(Cc4cccc(S(=O)(=O)N5CCC(Nc6ncc7c(n6)N([C@@H]6CCC[C@@H](O)C6)C(=O)C76CC6)CC5)c4)CC3)c21] | 853.4 | 1H NMR (400 MHz, DMSO-d6) δ 11.07 (s, 1H), 7.78-7.50 (m, 5H), 7.05 (s, 1H), 6.91 (dt, J = 15.6, 7.7 Hz, 3H), 5.34 (dd, J = 12.5, 5.3 Hz, 1H), 4.57 (d, J = 13.3 Hz, 1H), 4.50 (s, 1H), 4.03 (s, 1H), 3.69 (s, 2H), 3.62 (s, 3H), 3.57 (d, J = 12.3 Hz, 2H), 2.90 (dd, J = 31.7, 18.6 Hz, 7H), 2.75-2.56 (m, 3H), 2.47-2.27 (m, 4H), 2.14 (d, J = 12.5 Hz, 1H), 2.04-1.85 (m, 3H), 1.77- 1.66 (m, 2H), 1.66-1.43 (m, 8H), 1.39 (d, J = 3.9 Hz, 2H). |
| 182 | 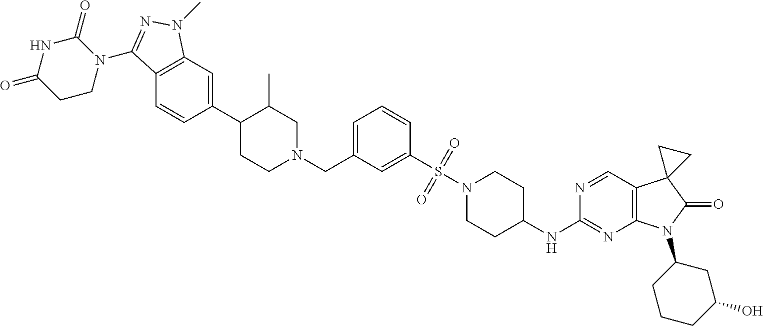 [SMILES: CC1CN(Cc2cccc(S(=O)(=O)N3CCC(Nc4ncc5c(n4)N([C@@H]4CCC[C@@H](O)C4)C(=O)C54CC4)CC3)c2)CCC1c1ccc2c(N3CCC(=O)NC3=O)nn(C)c2c1] | 851.3 | 1H NMR (400 MHz, DMSO-d6) δ 10.54 (s, 1H), 7.76 (s, 1H), 7.71-7.57 (m, 4H), 7.53 (d, J = 8.5 Hz, 1H), 7.33 (s, 1H), 7.06 (s, 1H), 6.99 (d, J = 8.6 Hz, 1H), 4.70-4.44 (m, 2H), 4.10-3.86 (m, 6H), 3.71 (d, J = 14.0 Hz, 1H), 3.53 (d, J = 14.2 Hz, 4H), 3.03 (d, J = 9.7 Hz, 1H), 2.95 (d, J = 12.2 Hz, 1H), 2.75 (t, J = 6.7 Hz, 3H), 2.42 (s, 3H), 2.35-2.04 (m, 6H), 1.91 (s, 2H), 1.59 (ddd, J = 44.2, 28.1, 14.0 Hz, 10H), 1.37 (d, J = 4.1 Hz, 2H), 0.74 (d, J = 6.9 Hz, 3H). |
| 183 | 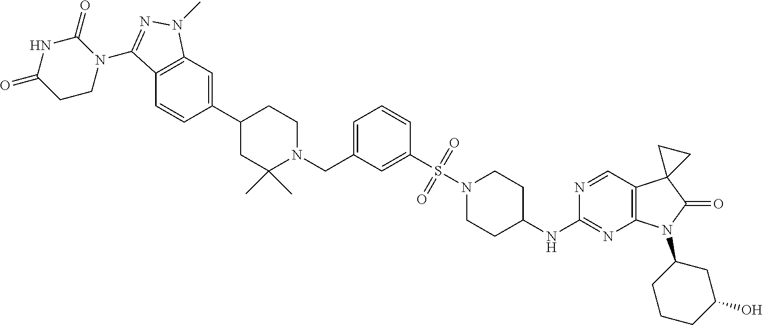 [SMILES: Cn1nc(N2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)c4)C(C)(C)C3)cc21] | 865.3 | 1H NMR (400 MHz, DMSO-d6) δ 10.54 (s, 1H), 7.74 (s, 2H), 7.69-7.55 (m, 3H), 7.53 (dd, J = 8.4, 1.9 Hz, 1H), 7.42 (s, 1H), 7.13-6.96 (m, 2H), 4.72-4.46 (m, 2H), 4.19 (d, J = 14.6 Hz, 1H), 4.03 (s, 1H), 3.95 (s, 3H), 3.90 (t, J = 6.7 Hz, 2H), 3.57 (s, 4H), 3.17 (d, J = 14.6 Hz, 1H), 2.96 (t, J = 12.5 Hz, 1H), 2.75 (t, J = 6.7 Hz, 2H), 2.49-2.36 (m, 4H), 2.20-2.05 (m, 1H), 1.90 (s, 2H), 1.81-1.65 (m, 4H), 1.52 (d, J = 44.0 Hz, 10H), 1.37 (d, J = 3.8 Hz, 2H), 1.26 (s, 3H), 1.16 (s, 3H). |
| 184 | 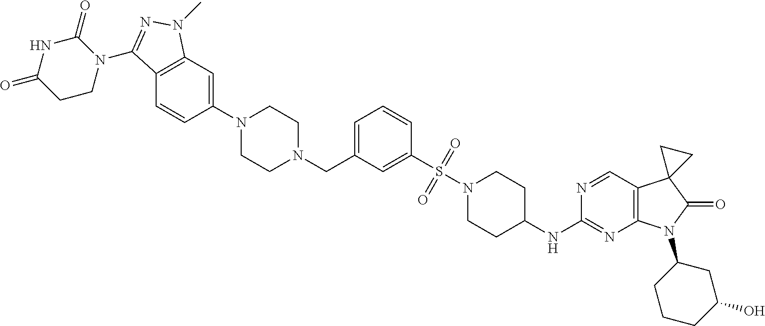 [SMILES: Cn1nc(N2CCC(=O)NC2=O)c2ccc(N3CCN(Cc4cccc(S(=O)(=O)N5CCC(Nc6ncc7c(n6)N([C@@H]6CCC[C@@H](O)C6)C(=O)C76CC6)CC5)c4)CC3)cc21] | 838.2 | 1H NMR (400 MHz, DMSO-d6) δ 10.50 (s, 1H), 7.68 (dt, J = 25.5, 7.9 Hz, 5H), 7.43 (d, J = 9.0 Hz, 1H), 7.07 (d, J = 7.2 Hz, 1H), 6.87 (d, J = 9.2 Hz, 1H), 6.82 (s, 1H), 4.69-4.47 (m, 2H), 4.04 (s, 1H), 3.94-3.81 (m, 5H), 3.68 (s, 2H), 3.57 (d, J = 11.5 Hz, 2H), 3.24 (d, J = 6.0 Hz, 4H), 2.73 (t, J = 6.6 Hz, 2H), 2.59 (d, J = 5.0 Hz, 4H), 2.44 (s, 2H), 2.15 (q, J = 12.4 Hz, 1H), 1.92 (s, 2H), 1.78-1.44 (m, 9H), 1.40 (q, J = 4.2, 3.7 Hz, 2H), 1.23 (s, 3H). |
| 185 | 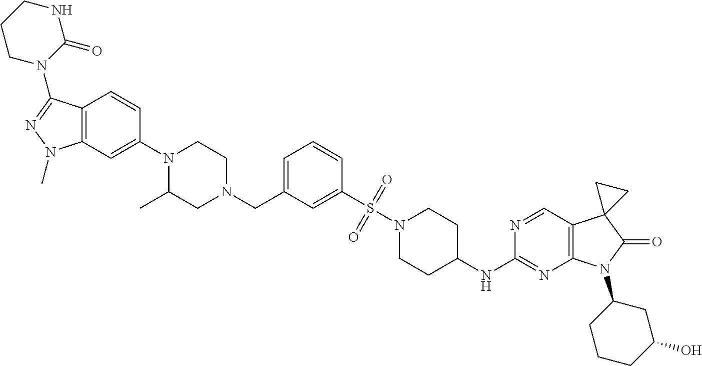 [SMILES: CC1CN(Cc2cccc(S(=O)(=O)N3CCC(Nc4ncc5c(n4)N([C@@H]4CCC[C@@H](O)C4)C(=O)C54CC4)CC3)c2)CCN1c1ccc2c(N3CCCNC3=O)nn(C)c2c1] | 852.5 | 1H NMR (400 MHz, DMSO-d6) δ 10.51 (s, 1H), 7.77 (s, 1H), 7.74-7.52 (m, 4H), 7.43 (d, J = 9.1 Hz, 1H), 7.07 (s, 1H), 6.87 (d, J = 9.2 Hz, 1H), 6.65 (d, J = 88.4 Hz, 1H), 4.59 (s, 1H), 4.53 (s, 1H), 4.13 (d, J = 7.1 Hz, 1H), 4.04 (s, 1H), 3.87 (d, J = 13.1 Hz, 5H), 3.81-3.73 (m, 2H), 3.56 (d, J = 13.7 Hz, 4H), 3.11 (t, J = 11.0 Hz, 1H), 2.92 (d, J = 10.8 Hz, 1H), 2.73 (t, J = 6.7 Hz, 2H), 2.70-2.63 (m, 2H), 2.41 (t, J = 11.6 Hz, 4H), 2.35-2.27 (m, 1H), 2.15 (d, J = 12.0 Hz, 1H), 1.92 (s, 2H), 1.78-1.44 (m, 9H), 1.40 (q, J = 4.0, 3.6 Hz, 2H), 1.03 (d, J = 6.3 Hz, 2H). |
| 186 | 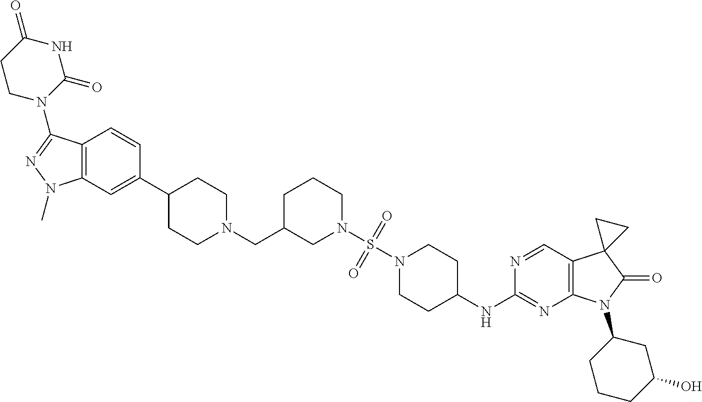 [SMILES: Cn1nc(N2CCC(=O)NC2=O)c2ccc(C3CCN(CC4CCCN(S(=O)(=O)N5CCC(Nc6ncc7c(n6)N([C@@H]6CCC[C@@H](O)C6)C(=O)C76CC6)CC5)C4)CC3)cc21] | 844.4 | 1H NMR (400 MHz, DMSO-d6) δ 10.54 (s, 1H), 7.71 (s, 1H), 7.54 (d, J = 8.4 Hz, 1H), 7.45 (s, 1H), 7.17-6.97 (m, 2H), 4.72-4.48 (m, 2H), 4.09 (s, 1H), 4.01-3.87 (m, 5H), 3.81 (s, 1H), 3.67-3.53 (m, 4H), 3.46 (d, J = 18.0 Hz, 3H), 3.03 (d, J = 10.6 Hz, 1H), 2.92 (d, J = 11.0 Hz, 3H), 2.88-2.71 (m, 3H), 2.69-2.55 (m, 3H), 2.21 (dd, J = 23.7, 7.5 Hz, 3H), 2.08 (s, 1H), 1.95 (d, J = 11.2 Hz, 3H), 1.88-1.58 (m, 9H), 1.53 (s, 6H), 1.38 (d, J = 27.9 Hz, 3H). |
| 187 | 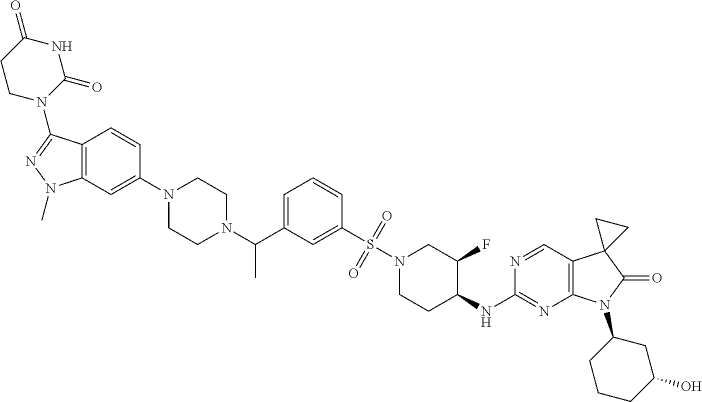 [SMILES: CC(c1cccc(S(=O)(=O)N2CC[C@H](Nc3ncc4c(n3)N([C@@H]3CCC[C@@H](O)C3)C(=O)C43CC3)[C@H](F)C2)c1)N1CCN(c2ccc3c(N4CCC(=O)NC4=O)nn(C)c3c2)CC1] | 870.3 | 1H NMR (400 MHz, DMSO-d6) δ 10.50 (s, 1H), 7.68 (dd, J = 31.7, 8.9 Hz, 5H), 7.43 (d, J = 9.1 Hz, 1H), 7.15 (s, 1H), 6.88 (d, J = 9.1 Hz, 1H), 6.79 (s, 1H), 4.92 (d, J = 48.8 Hz, 1H), 4.61 (s, 1H), 4.52 (d, J = 2.7 Hz, 1H), 4.12-3.79 (m, 8H), 3.72 (d, J = 11.2 Hz, 1H), 3.64 (dd, J = 6.7, 3.6 Hz, 1H), 3.50-3.38 (m, 2H), 3.22 (s, 4H), 2.79-2.69 (m, 3H), 2.69-2.53 (m, 4H), 2.16 (dd, J = 26.2, 12.9 Hz, 1H), 1.89 (dd, J = 25.8, 13.2 Hz, 1H), 1.80-1.67 (m, 2H), 1.66-1.45 (m, 6H), 1.39 (dd, J = 14.1, 7.1 Hz, 5H). |
| 188 | 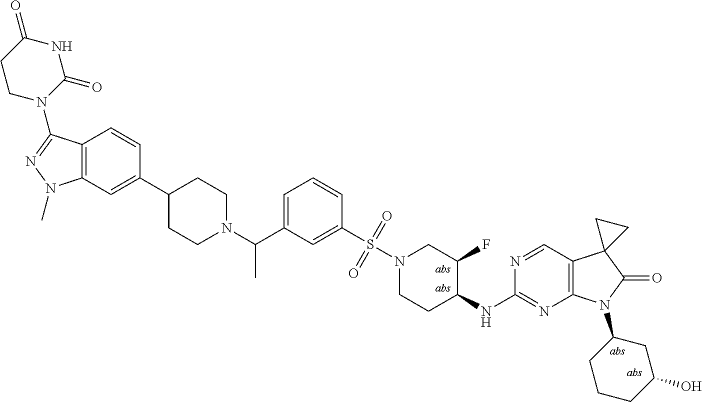 [SMILES: CC(c1cccc(S(=O)(=O)N2CC[C@H](Nc3ncc4c(n3)N([C@@H]3CCC[C@@H](O)C3)C(=O)C43CC3)[C@H](F)C2)c1)N1CCC(c2ccc3c(N4CCC(=O)NC4=O)nn(C)c3c2)CC1] | 869.4 | 1H NMR (400 MHz, DMSO-d6) δ 10.54 (s, 1H), 7.68 (dd, J = 31.9, 7.2 Hz, 5H), 7.53 (d, J = 8.4 Hz, 1H), 7.42 (s, 1H), 7.15 (d, J = 7.0 Hz, 1H), 7.01 (d, J = 8.5 Hz, 1H), 4.92 (d, J = 48.7 Hz, 1H), 4.60 (s, 1H), 4.52 (d, J = 2.7 Hz, 1H), 4.09- 3.80 (m, 8H), 3.78-3.70 (m, 1H), 3.67 (t, J = 6.2 Hz, 1H), 3.17-3.06 (m, 1H), 2.88 (d, J = 10.8 Hz, 1H), 2.75 (t, J = 6.8 Hz, 2H), 2.65- 2.54 (m, 2H), 2.54-2.52 (m, 1H), 2.48-2.38 (m, 1H), 2.21-1.97 (m, 3H), 1.97-1.67 (m, 7H), 1.66-1.25 (m, 12H). |
| 189 | 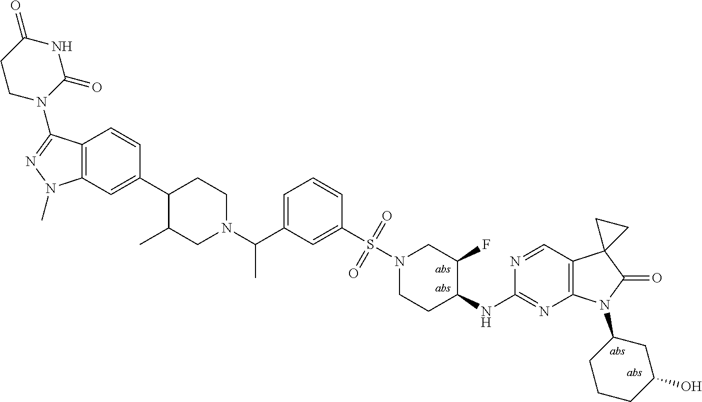 [SMILES: CC1CN(C(C)c2cccc(S(=O)(=O)N3CC[C@H](Nc4ncc5c(n4)N([C@@H]4CCC[C@@H](O)C4)C(=O)C54CC4)[C@H](F)C3)c2)CCC1c1ccc2c(N3CCC(=O)NC3=O)nn(C)c2c1] | 883.4 | 1H NMR (400 MHz, DMSO-d6) δ 10.53 (s, 1H), 7.84-7.57 (m, 5H), 7.53 (d, J = 8.4 Hz, 1H), 7.34-7.26 (m, 1H), 7.14 (s, 1H), 6.97 (d, J = 8.6 Hz, 1H), 4.92 (d, J = 48.5 Hz, 1H), 4.66-4.46 (m, 2H), 4.10-3.76 (m, 8H), 3.74-3.57 (m, 2H), 3.17 (s, 1H), 2.85 (dd, J = 28.7, 11.0 Hz, 2H), 2.74 (t, J = 6.7 Hz, 4H), 2.54 (s, 1H), 2.36 (s, 1H), 2.30-1.99 (m, 5H), 1.98-1.82 (m, 1H), 1.82-1.65 (m, 3H), 1.58 (d, J = 14.6 Hz, 3H), 1.51 (s, 3H), 1.43-1.38 (m, 2H), 1.35 (d, J = 6.6 Hz, 3H), 0.78-0.66 (m, 3H). |
| 190 | 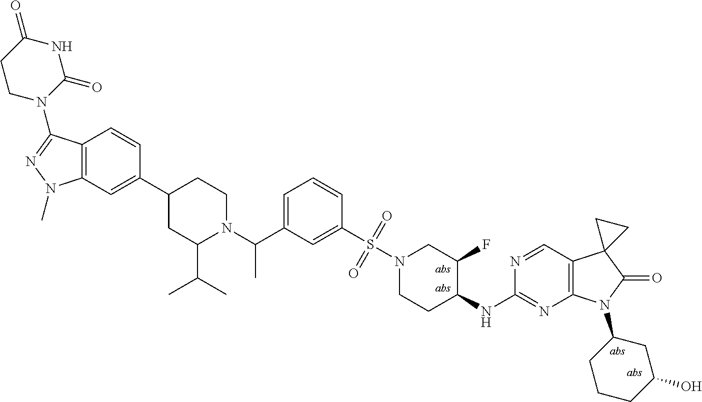 [SMILES: CC(C)C1CC(c2ccc3c(N4CCC(=O)NC4=O)nn(C)c3c2)CCN1C(C)c1cccc(S(=O)(=O)N2CC[C@H](Nc3ncc4c(n3)N([C@@H]3CCC[C@@H](O)C3)C(=O)C43CC3)[C@H](F)C2)c1] | 911.4 | 1H NMR (400 MHz, DMSO-d6) δ 10.54 (d, J = 3.0 Hz, 1H), 7.91-7.67 (m, 3H), 7.66-7.51 (m, 3H), 7.43 (dd, J = 18.8, 5.5 Hz, 1H), 7.21- 6.96 (m, 2H), 4.91 (d, J = 48.7 Hz, 1H), 4.67- 4.48 (m, 2H), 4.48-4.29 (m, 1H), 4.03 (s, 1H), 3.99 (d, J = 2.5 Hz, 2H), 3.97-3.82 (m, 3H), 3.72 (s, 1H), 3.22 (d, J = 14.5 Hz, 1H), 3.06- 2.90 (m, 1H), 2.89-2.62 (m, 4H), 2.60-2.52 (m, 1H), 2.44-2.25 (m, 3H), 2.15 (dd, J = 13.7, 6.4 Hz, 2H), 2.06-1.96 (m, 1H), 1.85 (dd, J = 24.6, 12.3 Hz, 3H), 1.77-1.67 (m, 3H), 1.64- 1.45 (m, 7H), 1.45-1.38 (m, 2H), 1.37-1.30 (m, 2H), 1.29 (d, J = 6.5 Hz, 1H), 1.12 (d, J = 6.3 Hz, 1H), 1.00-0.92 (m, 2H), 0.89-0.76 (m, 2H), 0.72 (d, J = 6.3 Hz, 1H). |
| 191 | 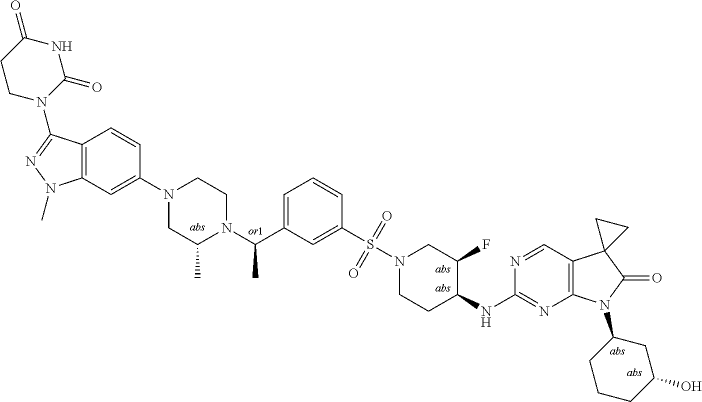 [SMILES: C[C@@H]1CN(c2ccc3c(N4CCC(=O)NC4=O)nn(C)c3c2)CCN1[C@H](C)c1cccc(S(=O)(=O)N2CC[C@H](Nc3ncc4c(n3)N([C@@H]3CCC[C@@H](O)C3)C(=O)C43CC3)[C@H](F)C2)c1] | 884.4 | 1H NMR (400 MHz, DMSO-d6) δ 10.50 (s, 1H), 7.67 (dd, J = 26.8, 6.0 Hz, 5H), 7.40 (d, J = 9.2 Hz, 1H), 7.16 (s, 1H), 6.85 (dd, J = 9.2, 2.0 Hz, 1H), 6.77 (d, J = 2.0 Hz, 1H), 4.92 (d, J = 48.8 Hz, 1H), 4.70-4.40 (m, 3H), 4.16 (q, J = 6.8 Hz, 1H), 4.12-3.92 (m, 3H), 3.86 (d, J = 9.0 Hz, 6H), 3.73 (d, J = 11.6 Hz, 1H), 3.21 (dd, J = 19.6, 9.1 Hz, 3H), 2.98 (d, J = 8.0 Hz, 1H), 2.89 (dd, J = 11.6, 6.8 Hz, 1H), 2.72 (t, J = 6.8 Hz, 2H), 2.15 (d, J = 13.6 Hz, 2H), 1.89 (q, J = 11.2 Hz, 1H), 1.74 (d, J = 13.2 Hz, 2H), 1.55 (d, J = 16.8 Hz, 7H), 1.44-1.31 (m, 5H), 1.24 (s, 2H), 1.13 (d, J = 6.0 Hz, 3H). |
| 192 | 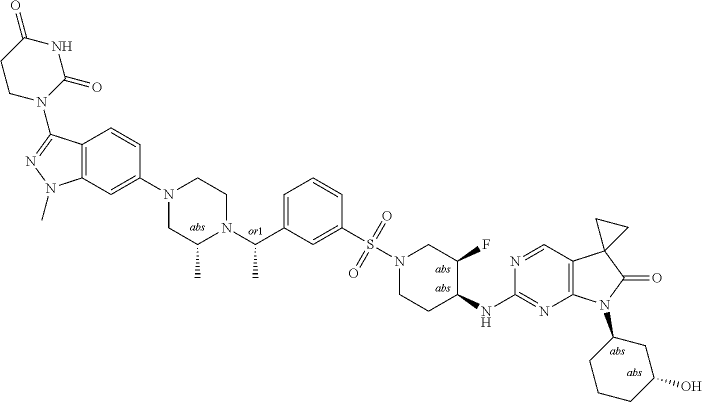 [SMILES: C[C@@H]1CN(c2ccc3c(N4CCC(=O)NC4=O)nn(C)c3c2)CCN1[C@@H](C)c1cccc(S(=O)(=O)N2CC[C@H](Nc3ncc4c(n3)N([C@@H]3CCC[C@@H](O)C3)C(=O)C43CC3)[C@H](F)C2)c1] | 884.4 | 1H NMR (400 MHz, DMSO-d6) δ 10.51 (s, 1H), 7.80 (d, J = 7.2 Hz, 2H), 7.64 (d, J = 5.2 Hz, 3H), 7.43 (d, J = 9.2 Hz, 1H), 7.16 (s, 1H), 6.89 (dd, J = 9.2, 2.0 Hz, 1H), 6.78 (s, 1H), 4.92 (d, J = 48.4 Hz, 1H), 4.61 (s, 1H), 4.54 (d, J = 2.8 Hz, 1H), 4.18 (d, J = 6.8 Hz, 1H), 4.00 (d, J = 43.2 Hz, 3H), 3.87 (d, J = 16.2 Hz, 6H), 3.71 (d, J = 11.2 Hz, 1H), 3.52 (s, 1H), 3.28 (s, 1H), 3.09 (s, 2H), 2.97-2.83 (m, 2H), 2.73 (t, J = 6.8 Hz, 3H), 2.40 (s, 2H), 2.16 (d, J = 12.8 Hz, 2H), 1.89 (q, J = 11.2 Hz, 1H), 1.71 (d, J = 12.4 Hz, 2H), 1.66-1.46 (m, 6H), 1.42 (t, J = 3.6 Hz, 2H), 1.32 (d, J = 6.4 Hz, 3H), 1.22 (d, J = 6.0 Hz, 3H). |
| 193 | 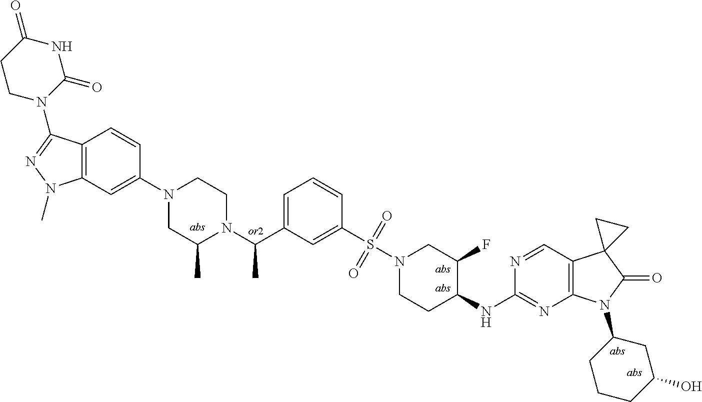 [SMILES: C[C@H](c1cccc(S(=O)(=O)N2CC[C@H](Nc3ncc4c(n3)N([C@@H]3CCC[C@@H](O)C3)C(=O)C43CC3)[C@H](F)C2)c1)N1CCN(c2ccc3c(N4CCC(=O)NC4=O)nn(C)c3c2)C[C@@H]1C] | 884.5 | 1H NMR (400 MHz, DMSO-d6) δ 10.51 (s, 1H), 7.74-7.61 (m, 5H), 7.40 (d, J = 9.0 Hz, 1H), 7.16 (s, 1H), 6.88-6.81 (m, 1H), 6.77 (s, 1H), 4.92 (d, J = 48.9 Hz, 2H), 4.68-4.49 (m, 3H), 4.17 (d, J = 6.8 Hz, 2H), 4.10-3.87 (m, 4H), 3.85 (s, 6H), 3.72 (d, J = 11.4 Hz, 3H), 3.25 (d, J = 9.8 Hz, 1H), 3.16 (s, 1H), 2.98 (s, 1H), 2.87 (dd, J = 11.3, 6.3 Hz, 1H), 2.72 (t, J = 6.7 Hz, 3H), 2.67 (t, J = 1.9 Hz, 1H), 2.15 (d, J = 12.5 Hz, 1H), 1.96-1.83 (m, 1H), 1.72 (s, 2H), 1.54 (d, J = 19.6 Hz, 5H), 1.44-1.40 (m, 4H), 1.23 (s, 3H). |
| 194 | 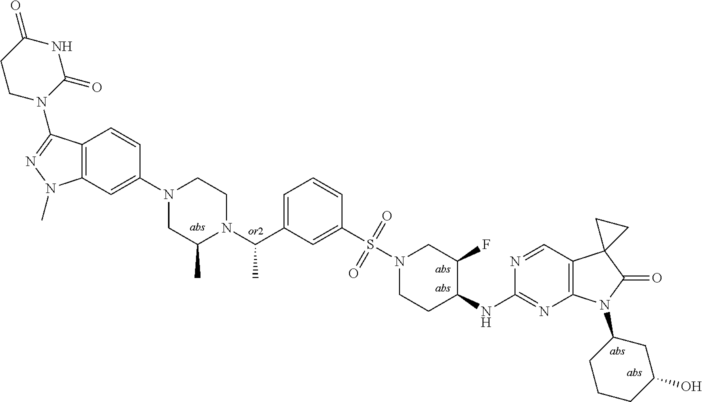 [SMILES: C[C@H]1CN(c2ccc3c(N4CCC(=O)NC4=O)nn(C)c3c2)CCN1[C@@H](C)c1cccc(S(=O)(=O)N2CC[C@H](Nc3ncc4c(n3)N([C@@H]3CCC[C@@H](O)C3)C(=O)C43CC3)[C@H](F)C2)c1] | 884.3 | 1H NMR (400 MHz, DMSO-d6) δ 10.51 (s, 1H), 7.79 (q, J = 3.8, 2.9 Hz, 2H), 7.74-7.53 (m, 3H), 7.43 (d, J = 9.1 Hz, 1H), 7.17 (s, 1H), 6.88 (dd, J = 9.1, 2.0 Hz, 1H), 6.78 (d, J = 1.9 Hz, 1H), 4.92 (d, J = 48.3 Hz, 1H), 4.68-4.47 (m, 2H), 4.17 (d, J = 6.7 Hz, 1H), 4.11-3.80 (m, 9H), 3.71 (d, J = 11.4 Hz, 1H), 3.57-3.45 (m, 1H), 3.25 (d, J = 11.4 Hz, 1H), 3.10 (s, 1H), 3.00 2.84 (m, 2H), 2.77-2.65 (m, 3H), 2.40 (s, 2H), 2.33 (p, J = 1.9 Hz, 2H), 2.16 (d, J = 12.7 Hz, 1H), 1.89 (q, J = 10.9, 10.1 Hz, 1H), 1.72 (t, J = 11.9 Hz, 2H), 1.66-1.46 (m, 6H), 1.42 (d, J = 3.8 Hz, 2H), 1.32 (d, J = 6.6 Hz, 3H), 1.22 (d, J = 6.2 Hz, 3H). |
| 195 | 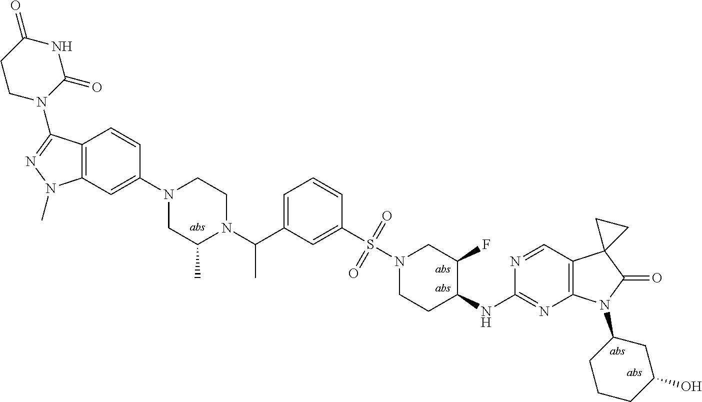 [SMILES: CC(c1cccc(S(=O)(=O)N2CC[C@H](Nc3ncc4c(n3)N([C@@H]3CCC[C@@H](O)C3)C(=O)C43CC3)[C@H](F)C2)c1)N1CCN(c2ccc3c(N4CCC(=O)NC4=O)nn(C)c3c2)C[C@H]1C] | 884.5 | 1H NMR (400 MHz, DMSO-d6) δ 10.50 (s, 1H), 7.80 (s, 1H), 7.76-7.56 (m, 4H), 7.42 (t, J = 9.2 Hz, 1H), 7.16 (s, 1H), 6.87 (dd, J = 16.0, 9.2 Hz, 1H), 6.77 (s, 1H), 4.92 (d, J = 48.8 Hz, 1H), 4.70-4.50 (m, 2H), 4.27-4.12 (m, 1H), 4.05 (s, 1H), 3.94 (s, 1H), 3.92-3.80 (m, 5H), 3.72 (s, 2H), 3.50 (d, J = 10.4 Hz, 1H), 3.27- 3.16 (m, 2H), 3.09 (s, 1H), 2.89 (dd, J = 12.0, 6.8 Hz, 3H), 2.73-2.70 (m, 2H), 2.40 (s, 1H), 2.16 (d, J = 13.2 Hz, 1H), 1.97-1.80 (m, 2H), 1.78-1.68 (m, 2H), 1.69-1.46 (m, 7H), 1.42 (d, J = 6.0 Hz, 3H), 1.32 (d, J = 6.4 Hz, 2H), 1.28-1.18 (m, 4H), 0.85 (t, J = 6.9 Hz, 1H). |
| 196 | 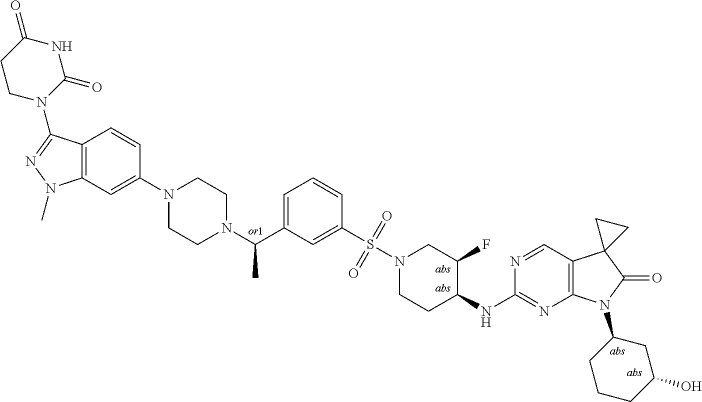 [SMILES: C[C@H](c1cccc(S(=O)(=O)N2CC[C@H](Nc3ncc4c(n3)N([C@@H]3CCC[C@@H](O)C3)C(=O)C43CC3)[C@H](F)C2)c1)N1CCN(c2ccc3c(N4CCC(=O)NC4=O)nn(C)c3c2)CC1] | 870.3 | 1H NMR (400 MHz, DMSO-d6) δ 10.51 (s, 1H), 7.80-7.58 (m, 5H), 7.43 (d, J = 9.0 Hz, 1H), 7.17 (s, 1H), 6.88 (dd, J = 9.1, 1.9 Hz, 1H), 6.79 (s, 1H), 4.92 (d, J = 48.4 Hz, 1H), 4.69-4.48 (m, 2H), 4.13-3.82 (m, 8H), 3.72 (d, J = 11.3 Hz, 1H), 3.68-3.58 (m, 1H), 3.32 (s, 2H), 3.22 (s, 4H), 2.73 (t, J = 6.7 Hz, 3H), 2.63 (d, J = 11.4 Hz, 4H), 2.16 (d, J = 12.5 Hz, 1H), 1.90 (q, J = 12.7, 12.3 Hz, 1H), 1.81-1.66 (m, 2H), 1.66-1.45 (m, 6H), 1.40 (dd, J = 16.5, 5.2 Hz, 5H). |
| 197 | 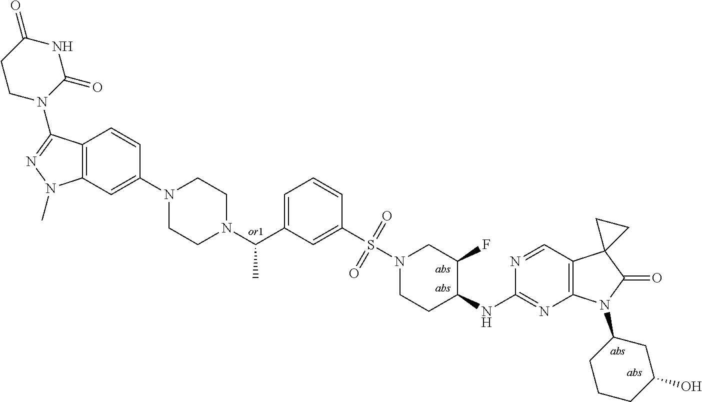 [SMILES: C[C@@H](c1cccc(S(=O)(=O)N2CC[C@H](Nc3ncc4c(n3)N([C@@H]3CCC[C@@H](O)C3)C(=O)C43CC3)[C@H](F)C2)c1)N1CCN(c2ccc3c(N4CCC(=O)NC4=O)nn(C)c3c2)CC1] | 870.3 | 1H NMR (400 MHz, DMSO-d6) δ 10.51 (s, 1H), 7.79-7.60 (m, 5H), 7.43 (d, J = 9.0 Hz, 1H), 7.17 (s, 1H), 6.88 (dd, J = 9.1, 1.9 Hz, 1H), 6.80 (d, J = 1.9 Hz, 1H), 4.92 (d, J = 48.7 Hz, 1H), 4.72-4.45 (m, 2H), 4.10-3.81 (m, 8H), 3.72 (d, J = 11.4 Hz, 1H), 3.64 (q, J = 6.6 Hz, 1H), 3.31 (s, 2H), 3.22 (d, J = 6.6 Hz, 4H), 2.73 (t, J = 6.7 Hz, 2H), 2.65-2.58 (m, 5H), 2.16 (d, J = 12.9 Hz, 1H), 1.98-1.82 (m, 1H), 1.80-1.66 (m, 2H), 1.66-1.45 (m, 6H), 1.40 (dd, J = 15.7, 5.2 Hz, 5H). |
| 198 | 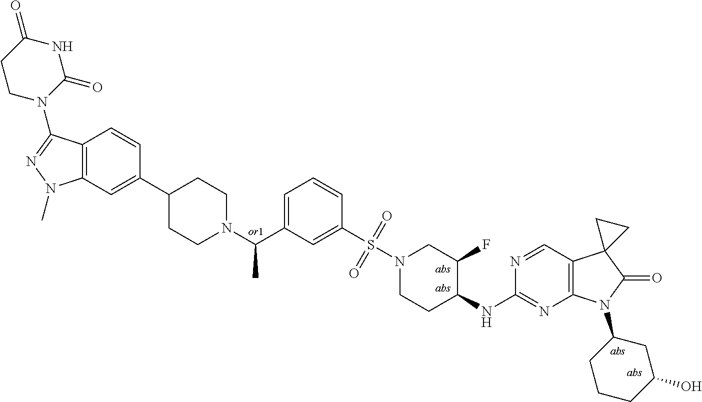 [SMILES: C[C@H](c1cccc(S(=O)(=O)N2CC[C@H](Nc3ncc4c(n3)N([C@@H]3CCC[C@@H](O)C3)C(=O)C43CC3)[C@H](F)C2)c1)N1CCC(c2ccc3c(N4CCC(=O)NC4=O)nn(C)c3c2)CC1] | 869.4 | 1H NMR (400 MHz, DMSO-d6) δ 10.53 (s, 1H), 7.67 (dd, J = 31.6, 6.9 Hz, 5H), 7.52 (d, J = 8.4 Hz, 1H), 7.42 (s, 1H), 7.13 (s, 1H), 7.01 (d, J = 8.5 Hz, 1H), 4.92 (d, J = 48.7 Hz, 1H), 4.60 (s, 1H), 4.51 (s, 1H), 4.12-3.78 (m, 8H), 3.78- 3.61 (m, 2H), 3.10 (d, J = 10.7 Hz, 1H), 2.88 (d, J = 10.7 Hz, 1H), 2.80-2.69 (m, 3H), 2.69- 2.55 (m, 2H), 2.24-1.98 (m, 3H), 1.95-1.66 (m, 8H), 1.54 (d, J = 43.5 Hz, 6H), 1.38 (t, J = 7.6 Hz, 5H). |
| 199 | 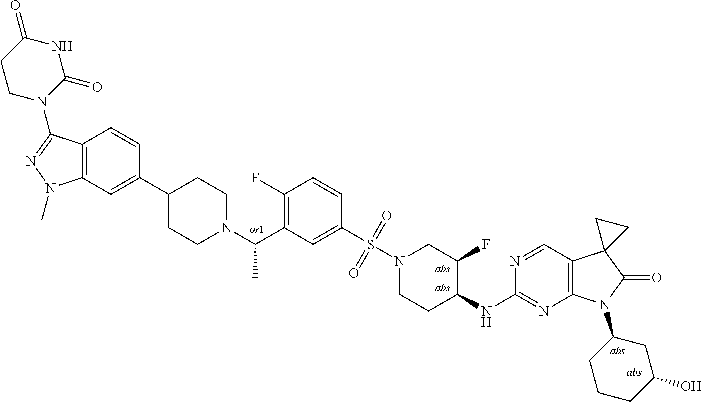 [SMILES: C[C@@H](c1cc(S(=O)(=O)N2CC[C@H](Nc3ncc4c(n3)N([C@@H]3CCC[C@@H](O)C3)C(=O)C43CC3)[C@H](F)C2)ccc1F)N1CCC(c2ccc3c(N4CCC(=O)NC4=O)nn(C)c3c2)CC1] | 869.4 | 1H NMR (400 MHz, DMSO-d6) δ 10.53 (s, 1H), 7.67 (dd, J = 31.7, 7.7 Hz, 5H), 7.53 (d, J = 8.4 Hz, 1H), 7.42 (s, 1H), 7.13 (d, J = 6.9 Hz, 1H), 7.01 (d, J = 8.5 Hz, 1H), 4.93 (d, J = 48.7 Hz, 1H), 4.60 (s, 1H), 4.51 (s, 1H), 3.92 (d, J = 18.5 Hz, 8H), 3.74 (d, J = 11.4 Hz, 1H), 3.66 (d, J = 6.9 Hz, 1H), 3.12 (d, J = 10.6 Hz, 1H), 2.87 (d, J = 10.7 Hz, 1H), 2.80-2.65 (m, 3H), 2.65-2.53 (m, 2H), 2.23-1.96 (m, 3H), 1.98- 1.66 (m, 8H), 1.66-1.44 (m, 6H), 1.39 (dd, J = 12.1, 5.5 Hz, 5H). |
| 200 | 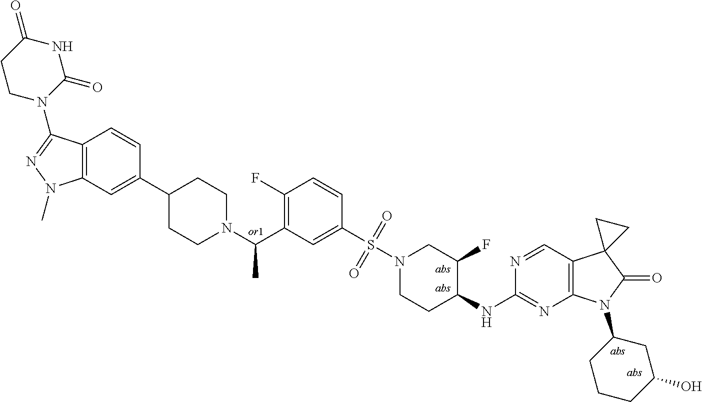 [SMILES: C[C@H](c1cc(S(=O)(=O)N2CC[C@H](Nc3ncc4c(n3)N([C@@H]3CCC[C@@H](O)C3)C(=O)C43CC3)[C@H](F)C2)ccc1F)N1CCC(c2ccc3c(N4CCC(=O)NC4=O)nn(C)c3c2)CC1] | 887.3 | 1H NMR (400 MHz, DMSO-d6) δ 10.54 (s, 1H), 7.91-7.81 (m, 1H), 7.73 (s, 1H), 7.57 (s, 1H), 7.54-7.44 (m, 2H), 7.42 (s, 1H), 7.13 (d, J = 7.3 Hz, 1H), 7.01 (d, J = 8.5 Hz, 1H), 4.91 (d, J = 48.7 Hz, 1H), 4.70-4.45 (m, 2H), 4.05 (s, 1H), 3.94 (s, 5H), 3.94-3.80 (m, 3H), 3.71 (d, J = 11.6 Hz, 1H), 3.20 (d, J = 10.9 Hz, 1H), 2.92 (d, J = 10.7 Hz, 1H), 2.83-2.67 (m, 3H), 2.65- 2.54 (m, 2H), 2.45 (s, 1H), 2.23-2.05 (m, 2H), 2.02-1.91 (m, 1H), 1.91-1.80 (m, 2H), 1.82- 1.62 (m, 5H), 1.62-1.40 (m, 6H), 1.39 (d, J = 6.1 Hz, 6H). |
| 201 | 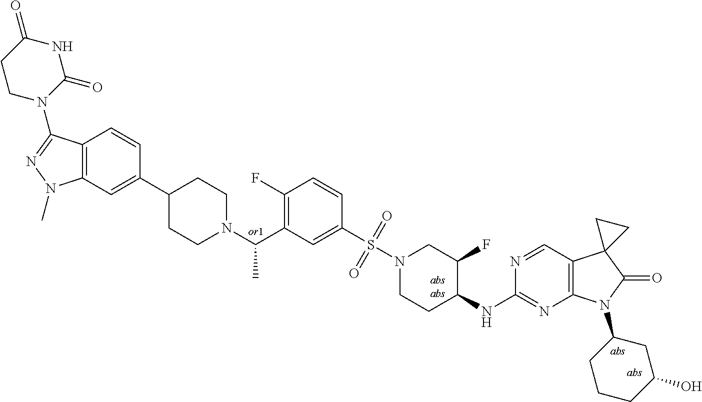 [SMILES: C[C@@H](c1cc(S(=O)(=O)N2CC[C@H](Nc3ncc4c(n3)N([C@@H]3CCC[C@@H](O)C3)C(=O)C43CC3)[C@H](F)C2)ccc1F)N1CCC(c2ccc3c(N4CCC(=O)NC4=O)nn(C)c3c2)CC1] | 887.3 | 1H NMR (400 MHz, DMSO-d6) δ 10.54 (s, 1H), 7.86 (d, J = 6.4 Hz, 1H), 7.69 (d, J = 26.0 Hz, 2H), 7.56-7.43 (m, 2H), 7.42 (s, 1H), 7.15 (s, 1H), 7.00 (d, J = 8.5 Hz, 1H), 4.91 (d, J = 47.8 Hz, 1H), 4.69-4.45 (m, 2H), 4.04 (s, 1H), 3.99- 3.79 (m, 8H), 3.75 (d, J = 11.6 Hz, 1H), 3.21 (d, J = 10.7 Hz, 1H), 2.91 (d, J = 10.8 Hz, 1H), 2.74 (t, J = 6.7 Hz, 3H), 2.69-2.54 (m, 2H), 2.47-2.32 (m, 1H), 2.13 (t, J = 11.0 Hz, 2H), 1.96 (t, J = 10.9 Hz, 1H), 1.93-1.80 (m, 3H), 1.74 (tdd, J = 16.4, 11.5, 2.7 Hz, 4H), 1.67- 1.44 (m, 6H), 1.39 (d, J = 6.8 Hz, 6H). |
| 202 | 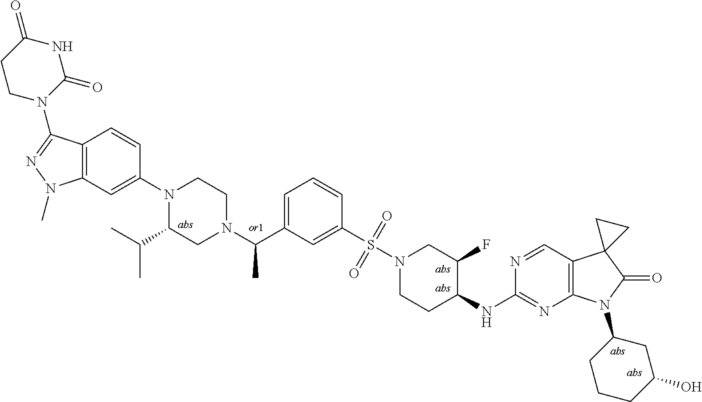 [SMILES: CC(C)[C@H]1CN([C@H](C)c2cccc(S(=O)(=O)N3CC[C@H](Nc4ncc5c(n4)N([C@@H]4CCC[C@@H](O)C4)C(=O)C54CC4)[C@H](F)C3)c2)CCN1c1ccc2c(N3CCC(=O)NC3=O)nn(C)c2c1] | 912.4 | 1H NMR (400 MHz, DMSO-d6) δ 10.49 (s, 1H), 7.84-7.56 (m, 5H), 7.36 (d, J = 9.0 Hz, 1H), 7.16 (s, 1H), 6.81 (d, J = 9.2 Hz, 1H), 6.66 (s, 1H), 4.93 (d, J = 48.6 Hz, 1H), 4.61 (s, 1H), 4.53 (s, 1H), 4.20-3.92 (m,2H), 3.87 (t, J = 6.7 Hz, 2H), 3.81 (s, 3H), 3.77-3.49 (m, 5H), 3.20 (t, J = 11.9 Hz, 2H), 2.97 (d, J = 11.1 Hz, 1H), 2.79- 2.61 (m, 4H), 2.31-2.04 (m, 3H), 1.92 (d, J = 12.8 Hz, 1H), 1.75 (d, J = 12.3 Hz, 2H), 1.67- 1.28 (m, 13H), 0.82 (d, J = 6.5 Hz, 3H), 0.72 (d, J = 6.7 Hz, 3H). |
| 203 | 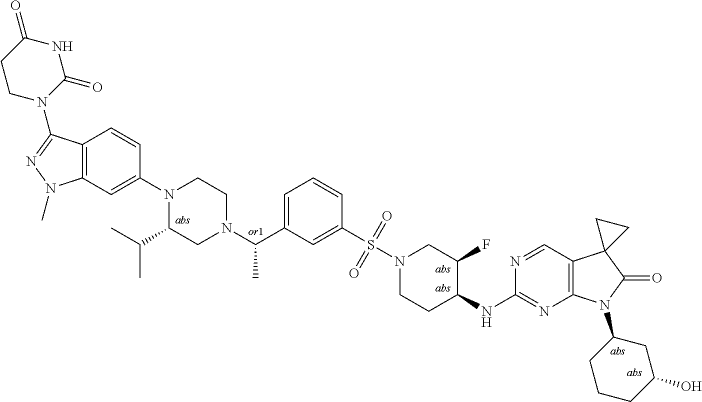 [SMILES: CC(C)[C@H]1CN([C@@H](C)c2cccc(S(=O)(=O)N3CC[C@H](Nc4ncc5c(n4)N([C@@H]4CCC[C@@H](O)C4)C(=O)C54CC4)[C@H](F)C3)c2)CCN1c1ccc2c(N3CCC(=O)NC3=O)nn(C)c2c1] | 912.4 | 1H NMR (400 MHz, DMSO-d6) δ 10.48 (s, 1H), 7.79-7.56 (m, 5H), 7.35 (d, J = 9.1 Hz, 1H), 7.15 (d, J = 7.2 Hz, 1H), 6.87-6.74 (m, 1H), 6.66 (s, 1H), 4.92 (d, J = 48.6 Hz, 1H), 4.71- 4.49 (m, 2H), 4.14-3.91 (m, 2H), 3.87 (t, J = 6.7 Hz, 2H), 3.82 (s, 3H), 3.74 (d, J = 11.4 Hz, 1H), 3.66 (d, J = 12.9 Hz, 1H), 3.56 (d, J = 9.5 Hz, 1H), 3.50 (d, J = 6.7 Hz, 1H), 3.31-3.19 (m, 2H), 3.00 (d, J = 10.3 Hz, 1H), 2.71 (t, J = 6.9 Hz, 3H), 2.66-2.53 (m, 1H), 2.53 (s, 1H), 2.48 (s, 1H), 2.15 (t, J = 10.8 Hz, 2H), 2.05 (d, J = 10.4 Hz, 1H), 1.88 (t, J = 12.0 Hz, 1H), 1.82- 1.67 (m, 2H), 1.67-1.46 (m, 7H), 1.42 (q, J = 4.0, 3.6 Hz, 2H), 1.35 (d, J = 6.7 Hz, 3H), 0.70 (dd, J = 12.1, 6.6 Hz, 6H). |
| 204 | 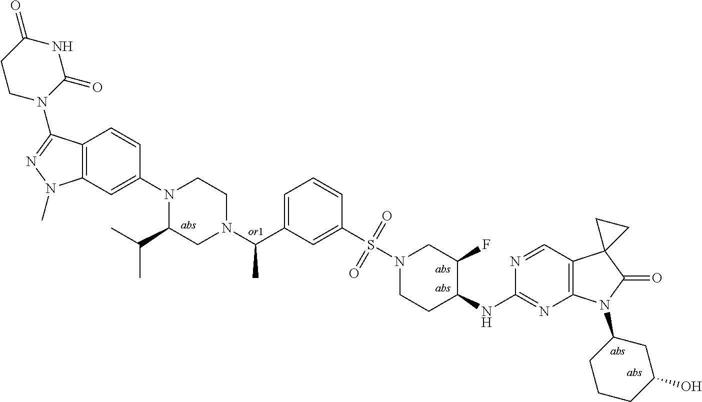 [SMILES: CC(C)[C@@H]1CN([C@H](C)c2cccc(S(=O)(=O)N3CC[C@H](Nc4ncc5c(n4)N([C@@H]4CCC[C@@H](O)C4)C(=O)C54CC4)[C@H](F)C3)c2)CCN1c1ccc2c(N3CCC(=O)NC3=O)nn(C)c2c1] | 912.4 | 1H NMR (400 MHz, DMSO-d6) δ 10.48 (s, 1H), 7.87-7.52 (m, 5H), 7.36 (d, J = 9.0 Hz, 1H), 7.13 (d, J = 7.4 Hz, 1H), 6.81 (dd, J = 9.3, 2.0 Hz, 1H), 6.66 (s, 1H), 4.93 (d, J = 48.8 Hz, 1H), 4.72-4.42 (m, 2H), 4.01 (d, J = 30.4 Hz, 2H), 3.87 (t, J = 6.7 Hz, 3H), 3.81 (s, 3H), 3.73 (d, J = 11.6 Hz, 1H), 3.63 (d, J = 7.3 Hz, 2H), 3.56 (d, J = 12.6 Hz, 1H), 3.20 (t, J = 11.8 Hz, 1H), 2.98 (d, J = 11.1 Hz, 1H), 2.78-2.56 (m, 5H), 2.28- 2.10 (m, 3H), 1.95-1.83 (m, 1H), 1.73 (t, J = 11.5 Hz, 2H), 1.65-1.46 (m, 6H), 1.38 (dd, J = 31.7, 5.1 Hz, 6H), 1.21 (d, J = 23.3 Hz, 2H), 0.83 (d, J = 6.5 Hz, 3H), 0.72 (d, J = 6.7 Hz, 3H). |
| 205 | 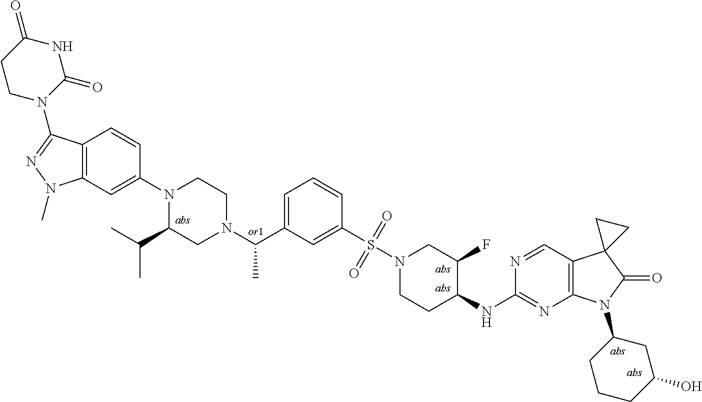 [SMILES: CC(C)[C@@H]1CN([C@@H](C)c2cccc(S(=O)(=O)N3CC[C@H](Nc4ncc5c(n4)N([C@@H]4CCC[C@@H](O)C4)C(=O)C54CC4)[C@H](F)C3)c2)CCN1c1ccc2c(N3CCC(=O)NC3=O)nn(C)c2c1] | 912.3 | 1H NMR (400 MHz, DMSO-d6) δ 10.47 (s, 1H), 7.87-7.53 (m, 5H), 7.35 (d, J = 9.0 Hz, 1H), 7.13 (d, J = 7.1 Hz, 1H), 6.82-6.68 (m, 1H), 6.66 (s, 1H), 4.93 (d, J = 49.0 Hz, 1H), 4.68- 4.43 (m, 2H), 4.01 (d, J = 30.9 Hz, 3H), 3.87 (t, J = 6.7 Hz, 2H), 3.82 (s, 3H), 3.76-3.65 (m, 2H), 3.57-3.47 (m, 2H), 3.00 (d, J = 10.3 Hz, 1H), 2.71 (t, J = 6.5 Hz, 4H), 2.20-2.03 (m, 3H), 1.90 (q, J = 11.3 Hz, 1H), 1.73 (s, 2H), 1.57 (d, J = 25.3 Hz, 6H), 1.42 (q, J = 3.7 Hz, 2H), 1.35 (d, J = 6.6 Hz, 3H), 1.24 (s, 2H), 0.84 (d, J = 7.2 Hz, 1H), 0.70 (dd, J = 13.6, 6.6 Hz, 6H). |
| 206 | 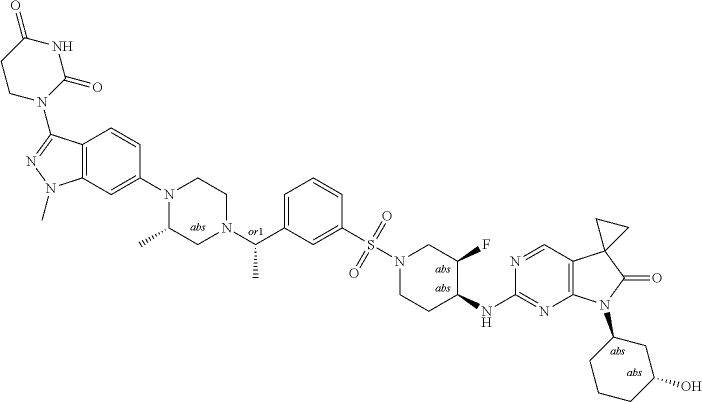 [SMILES: C[C@@H](c1cccc(S(=O)(=O)N2CC[C@H](Nc3ncc4c(n3)N([C@@H]3CCC[C@@H](O)C3)C(=O)C43CC3)[C@H](F)C2)c1)N1CCN(c2ccc3c(N4CCC(=O)NC4=O)nn(C)c3c2)[C@@H](C)C1] | 884.4 | 1H NMR (400 MHz, DMSO-d6) δ 10.50 (s, 1H), 7.92-7.57 (m, 5H), 7.43 (d, J = 9.0 Hz, 1H), 7.16 (s, 1H), 6.87 (dd, J = 9.3, 1.9 Hz, 1H), 6.73 (s, 1H), 4.92 (d, J = 48.8 Hz, 1H), 4.73-4.38 (m, 2H), 4.21-3.92 (m, 2H), 3.92-3.85 (m, 3H), 3.83 (s, 3H), 3.74 (d, J = 11.5 Hz, 1H), 3.66 (s, 1H), 3.29-3.21 (m, 1H), 3.05 (d, J = 11.8 Hz, 1H), 2.74 (q, J = 12.1, 6.7 Hz, 4H), 2.65- 2.53 (m, 2H), 2.48-2.38 (m, 2H), 2.31-2.21 (m, 1H), 2.21-2.07 (m, 1H), 1.98-1.83 (m, 1H), 1.80-1.67 (m, 2H), 1.67-1.45 (m, 6H), 1.45-1.29 (m, 5H), 1.05 (d, J = 6.3 Hz, 3H). |
| 207 | 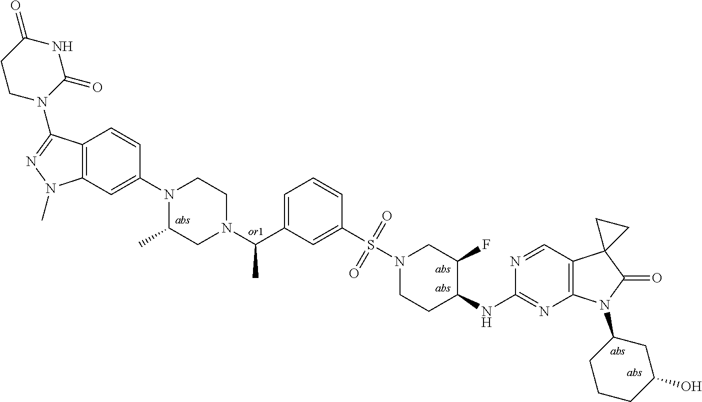 [SMILES: C[C@H](c1cccc(S(=O)(=O)N2CC[C@H](Nc3ncc4c(n3)N([C@@H]3CCC[C@@H](O)C3)C(=O)C43CC3)[C@H](F)C2)c1)N1CCN(c2ccc3c(N4CCC(=O)NC4=O)nn(C)c3c2)[C@@H](C)C1] | 884.4 | 1H NMR (400 MHz, DMSO-d6) δ 10.50 (s, 1H), 7.85-7.56 (m, 5H), 7.42 (d, J = 9.1 Hz, 1H), 7.13 (s, 1H), 6.86 (d, J = 9.2 Hz, 1H), 6.73 (s, 1H), 4.91 (d, J = 48.8 Hz, 1H), 4.59 (d, J = 15.9 Hz, 2H), 4.07 (s, 2H), 3.87 (d, J = 12.1 Hz, 4H), 3.72 (s, 3H), 3.58 (d, J = 7.0 Hz, 2H), 3.15- 3.03 (m, 3H), 2.73 (t, J = 6.7 Hz, 3H), 2.61- 2.52 (m, 2H), 2.33 (d, J = 9.8 Hz, 2H), 2.27- 2.04 (m, 3H), 1.89 (d, J = 12.9 Hz, 1H), 1.80- 1.66 (m, 1H), 1.55 (d, J = 14.0 Hz, 7H), 1.42 (q, J = 4.1, 3.7 Hz, 2H), 1.37 (d, J = 6.6 Hz, 3H), 1.00 (d, J = 6.3 Hz, 3H). |
| 208 | 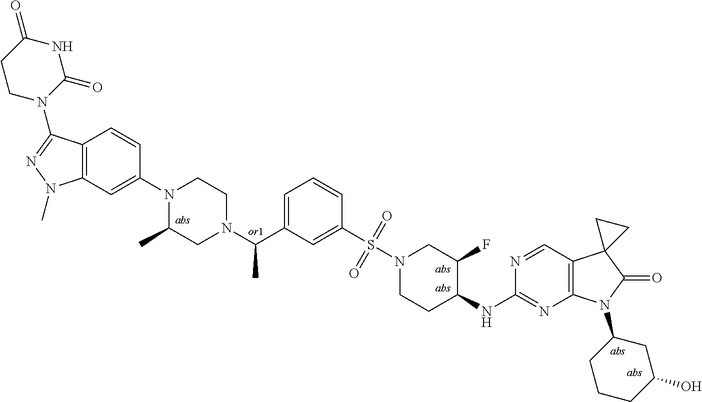 [SMILES: C[C@H](c1cccc(S(=O)(=O)N2CC[C@H](Nc3ncc4c(n3)N([C@@H]3CCC[C@@H](O)C3)C(=O)C43CC3)[C@H](F)C2)c1)N1CCN(c2ccc3c(N4CCC(=O)NC4=O)nn(C)c3c2)[C@H](C)C1] | 884.4 | 1H NMR (400 MHz, DMSO-d6) δ 10.50 (s, 1H), 7.85-7.59 (m, 5H), 7.43 (d, J = 9.1 Hz, 1H), 7.16 (d, J = 7.2 Hz, 1H), 6.87 (dd, J = 9.3, 1.9 Hz, 1H), 6.79-6.70 (m, 1H), 4.93 (d, J = 48.8 Hz, 1H), 4.61 (s, 1H), 4.52 (d, J = 2.7 Hz, 1H), 4.08 (dd, J = 52.6, 20.3 Hz, 3H), 3.92-3.78 (m, 6H), 3.69 (dd, J = 23.8, 8.9 Hz, 2H), 3.28 (d, J = 11.7 Hz, 1H), 3.04 (t, J = 11.0 Hz, 1H), 2.83- 2.70 (m, 5H), 2.61 (d, J = 47.4 Hz, 1H), 2.43 (d, J = 8.3 Hz, 1H), 2.31-2.06 (m, 2H), 1.88 (t, J = 11.7 Hz, 1H), 1.73 (t, J = 12.3 Hz, 2H), 1.67- 1.45 (m, 6H), 1.41 (d, J = 3.8 Hz, 2H), 1.36 (d, J = 6.6 Hz, 3H), 1.06 (d, J = 6.3 Hz, 3H). |
| 209 | 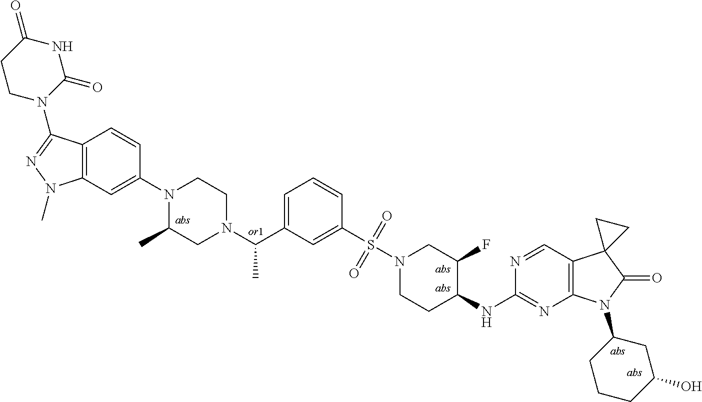 [SMILES: C[C@@H]1CN([C@@H](C)c2cccc(S(=O)(=O)N3CC[C@H](Nc4ncc5c(n4)N([C@@H]4CCC[C@@H](O)C4)C(=O)C54CC4)[C@H](F)C3)c2)CCN1c1ccc2c(N3CCC(=O)NC3=O)nn(C)c2c1] | 884.4 | 1H NMR (400 MHz, DMSO-d6) δ 10.50 (s, 1H), 7.86-7.56 (m, 5H), 7.43 (d, J = 9.0 Hz, 1H), 7.16 (s, 1H), 6.85 (dd, J = 9.4, 1.9 Hz, 1H), 6.74 (s, 1H), 5.12-4.78 (m, 1H), 4.61 (s, 1H), 4.53 (d, J = 2.7 Hz, 1H), 4.06 (t, J = 11.3 Hz, 2H), 4.02-3.79 (m, 7H), 3.71 (d, J = 11.6 Hz, 1H), 3.63-3.49 (m, 1H), 3.41 (d, J = 11.5 Hz, 1H), 3.12 (d, J = 10.5 Hz, 2H), 2.72 (t, J = 6.7 Hz, 3H), 2.56 (t, J = 8.8 Hz, 1H), 2.49-2.36 (m, 1H), 2.35-2.28 (m, 1H), 2.29-2.05 (m, 2H), 1.91 (q, J = 11.9 Hz, 1H), 1.71 (d, J = 13.1 Hz, 2H), 1.55 (q, J = 20.6, 16.3 Hz, 6H), 1.45-1.29 (m, 6H), 1.00 (d, J = 6.3 Hz, 3H). |
| 210 | 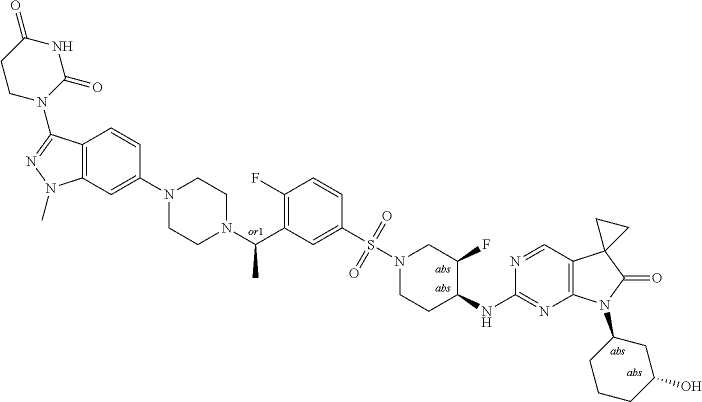 [SMILES: C[C@H](c1cc(S(=O)(=O)N2CC[C@H](Nc3ncc4c(n3)N([C@@H]3CCC[C@@H](O)C3)C(=O)C43CC3)[C@H](F)C2)ccc1F)N1CCN(c2ccc3c(N4CCC(=O)NC4=O)nn(C)c3c2)CC1] | 888.3 | 1H NMR (400 MHz, DMSO-d6) δ 10.48 (s, 1H), 7.87 (dd, J = 6.8, 2.4 Hz, 1H), 7.70 (d, J = 26.8 Hz, 2H), 7.58-7.37 (m, 2H), 7.14 (d, J = 7.3 Hz, 1H), 6.91-6.68 (m, 2H), 4.91 (d, J = 48.8 Hz, 1H), 4.56 (d, J = 38.8 Hz, 2H), 4.06 (s, 1H), 4.00-3.78 (m, 8H), 3.71 (d, J = 11.9 Hz, 1H), 3.23 (t, J = 5.0 Hz, 4H), 2.80 (d, J = 13.3 Hz, 1H), 2.72 (q, J = 6.1, 5.6 Hz, 2H), 2.69-2.59 (m, 3H), 2.55 (d, J = 5.6 Hz, 2H), 2.16 (q, J = 12.1 Hz, 1H), 1.92-1.67 (m, 3H), 1.58 (dt, J = 22.2, 17.1 Hz, 7H), 1.47-1.34 (m, 6H). |
| 211 | 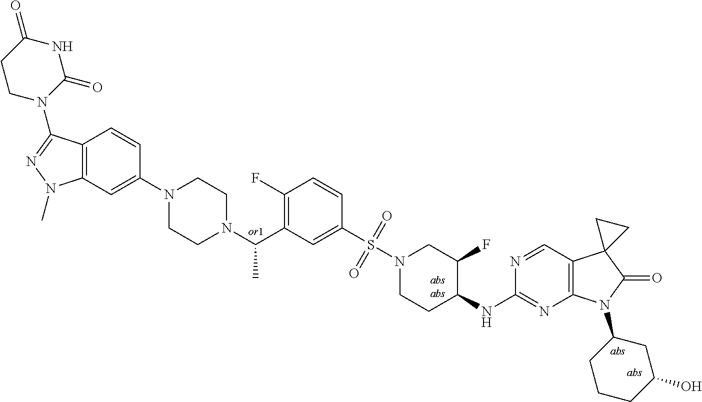 [SMILES: C[C@@H](c1cc(S(=O)(=O)N2CC[C@H](Nc3ncc4c(n3)N([C@@H]3CCC[C@@H](O)C3)C(=O)C43CC3)[C@H](F)C2)ccc1F)N1CCN(c2ccc3c(N4CCC(=O)NC4=O)nn(C)c3c2)CC1] | 888.3 | 1H NMR (400 MHz, DMSO-d6) δ 10.44 (s, 1H), 7.88 (dd, J = 6.5, 2.4 Hz, 1H), 7.71 (d, J = 11.8 Hz, 2H), 7.50 (dd, J = 9.8, 8.6 Hz, 1H), 7.42 (d, J = 9.0 Hz, 1H), 7.15 (s, 1H), 6.87 (dd, J = 9.2, 1.9 Hz, 1H), 6.79 (d, J = 1.9 Hz, 1H), 4.92 (d, J = 48.5 Hz, 1H), 4.57 (d, J = 37.1 Hz, 2H), 4.05 (s, 1H), 4.01-3.81 (m, 8H), 3.72 (d, J = 11.8 Hz, 1H), 3.23 (d, J = 5.2 Hz, 4H), 2.72 (t, J = 6.7 Hz, 2H), 2.69-2.60 (m, 3H), 2.56 (dd, J = 14.3, 3.7 Hz, 3H), 2.15 (d, J = 12.7 Hz, 1H), 1.88 (q, J = 12.1 Hz, 1H), 1.72 (dd, J = 13.8, 10.1 Hz, 2H), 1.68-1.45 (m, 7H), 1.41 (dd, J = 7.6, 5.2 Hz, 6H). |

### Biological Assays

### HiBit Assay Protocol for CDK2

### 1. Materials

1.1 Reagents

| Reagents | Vendor | Cat# |
|---|---|---|
| HEK293-CDK2-Hibit cell | Pharmaron | \ |
| DMEM | Hyclone | SH30022 01 |
| FBS | Gibco | 10099141C |
| Nano-Glo ® HiBiT Lytic Detection | Promega | N3040 |
| System | | |
| 0.25% Trypsin/EDTA | Invitrogen | #25300 |
| PBS | Solarbio | P1020-500 |
| Penicillin-Streptomycin Liquid | Solarbio | P1400 |

1.2 Instruments

| Instrument | Vendor | Cat# |
|---|---|---|
| Centrifuge | Eppendorf | 5810R |
| CO₂ Incubator | Thermo | Model: 371 |
| Vortex | IKA | MS3 digital |
| Microplate shaker | Yoning | WZ-4 |
| Echo Liquid Handler | Labcyte | 550 |
| TECAN | TECAN | Freedom EVO200 |
| 384 well plate | Corning | 3570 |
| 384 pp-plate | Labcyte | 001-14555 |
| 50 mL centrifuge tube | BD-Falcon | 352098 |
| 15 mL centrifuge tube | BD-Falcon | 352097 |
| EnVision | PerkinElmer | 2105-0020 |

### 2. Assay Procedure

### Day 1.

1. The culture medium for HEK293-CDK2-Hibit cells was prepared using DMEM with 10% FBS.
2. Cells were cultivated in T-75 flasks in a cell culture incubator set at 37° C., 5% CO2, 95% relative humidity. Cells were allowed to reach 80-90% confluence before detaching and splitting.
3. Cultivated cells were rinsed in T-75 flasks with 5 mL PBS. Aspirate off Added was 1.5 mL trypsin, and the cells were incubated at 37° C. for approximately 5 minutes or until the cells detached and floated.
4. Trypsin was inactivated by adding excess serum containing medium.
5. Cells were harvested from flask into cell culture medium and then the cell number was counted. HEK293-CDK2-Hibit cells were seeded into 384-well plate (cell density: 1.6×105 cells/well/50 uL medium) in DMEM medium according to the plate map below and incubated overnight at 37° C. and 5% CO2.
Note: Cell Cultured Medium: 90% DMEM+10% FBS+1% Penicillin-Streptomycin Liquid

Plate map

| Plate | | | | | | | | | | | | | |
|---|---|---|---|---|---|---|---|---|---|---|---|---|---|
| map | | | | | | | | | | | | | |
| Top(nM) | | 1 | 2 | 3 | 4 | 5 | 6 | 7 | 8 | 9 | 10 | 11 | 12 |
| | A | | | | | | | | | | | | |
| 10000 | B | NC | 10000 | 3333 | 1111 | 370 | 123 | 41.2 | 13.7 | 4.6 | 1.5 | 0.51 | PC |
| 10000 | C | NC | 10000 | 3333 | 1111 | 370 | 123 | 41.2 | 13.7 | 4.6 | 1.5 | 0.51 | PC |
| 10000 | D | NC | 10000 | 3333 | 1111 | 370 | 123 | 41.2 | 13.7 | 4.6 | 1.5 | 0.51 | PC |
| 10000 | E | NC | 10000 | 3333 | 1111 | 370 | 123 | 41.2 | 13.7 | 4.6 | 1.5 | 0.51 | PC |
| 10000 | F | NC | 10000 | 3333 | 1111 | 370 | 123 | 41.2 | 13.7 | 4.6 | 1.5 | 0.51 | PC |
| 10000 | G | NC | 10000 | 3333 | 1111 | 370 | 123 | 41.2 | 13.7 | 4.6 | 1.5 | 0.51 | PC |
| 10000 | H | NC | 10000 | 3333 | 1111 | 370 | 123 | 41.2 | 13.7 | 4.6 | 1.5 | 0.51 | PC |
| 10000 | I | NC | 10000 | 3333 | 1111 | 370 | 123 | 41.2 | 13.7 | 4.6 | 1.5 | 0.51 | PC |
| 10000 | J | NC | 10000 | 3333 | 1111 | 370 | 123 | 41.2 | 13.7 | 4.6 | 1.5 | 0.51 | PC |
| 10000 | K | NC | 10000 | 3333 | 1111 | 370 | 123 | 41.2 | 13.7 | 4.6 | 1.5 | 0.51 | PC |
| 10000 | L | NC | 10000 | 3333 | 1111 | 370 | 123 | 41.2 | 13.7 | 4.6 | 1.5 | 0.51 | PC |
| 10000 | M | NC | 10000 | 3333 | 1111 | 370 | 123 | 41.2 | 13.7 | 4.6 | 1.5 | 0.51 | PC |
| 10000 | N | NC | 10000 | 3333 | 1111 | 370 | 123 | 41.2 | 13.7 | 4.6 | 1.5 | 0.51 | PC |
| 10000 | O | NC | 10000 | 3333 | 1111 | 370 | 123 | 41.2 | 13.7 | 4.6 | 1.5 | 0.51 | PC |
| | P | | | | | | | | | | | | |
| Plate | | | | | | | | | | | | | |
| map | | | | | | | | | | | | | |
| Top(nM) | | 13 | 14 | 15 | 16 | 17 | 18 | 19 | 20 | 21 | 22 | 23 | 24 |
| | A | | | | | | | | | | | | |
| 10000 | B | PC | 10000 | 3333 | 1111 | 370 | 123 | 41.2 | 13.7 | 4.6 | 1.5 | 0.51 | NC |
| 10000 | C | PC | 10000 | 3333 | 1111 | 370 | 123 | 41.2 | 13.7 | 4.6 | 1.5 | 0.51 | NC |
| 10000 | D | PC | 10000 | 3333 | 1111 | 370 | 123 | 41.2 | 13.7 | 4.6 | 1.5 | 0.51 | NC |
| 10000 | E | PC | 10000 | 3333 | 1111 | 370 | 123 | 41.2 | 13.7 | 4.6 | 1.5 | 0.51 | NC |
| 10000 | F | PC | 10000 | 3333 | 1111 | 370 | 123 | 41.2 | 13.7 | 4.6 | 1.5 | 0.51 | NC |
| 10000 | G | PC | 10000 | 3333 | 1111 | 370 | 123 | 41.2 | 13.7 | 4.6 | 1.5 | 0.51 | NC |
| 10000 | H | PC | 10000 | 3333 | 1111 | 370 | 123 | 41.2 | 13.7 | 4.6 | 1.5 | 0.51 | NC |
| 10000 | I | PC | 10000 | 3333 | 1111 | 370 | 123 | 41.2 | 13.7 | 4.6 | 1.5 | 0.51 | NC |
| 10000 | J | PC | 10000 | 3333 | 1111 | 370 | 123 | 41.2 | 13.7 | 4.6 | 1.5 | 0.51 | NC |
| 10000 | K | PC | 10000 | 3333 | 1111 | 370 | 123 | 41.2 | 13.7 | 4.6 | 1.5 | 0.51 | NC |
| 10000 | L | PC | 10000 | 3333 | 1111 | 370 | 123 | 41.2 | 13.7 | 4.6 | 1.5 | 0.51 | NC |
| 10000 | M | PC | 10000 | 3333 | 1111 | 370 | 123 | 41.2 | 13.7 | 4.6 | 1.5 | 0.51 | NC |
| 10000 | N | PC | 10000 | 3333 | 1111 | 370 | 123 | 41.2 | 13.7 | 4.6 | 1.5 | 0.51 | NC |
| 10000 | O | PC | 10000 | 3333 | 1111 | 370 | 123 | 41.2 | 13.7 | 4.6 | 1.5 | 0.51 | NC |
| | P | | | | | | | | | | | | |

### Day 2.

1. Test compounds were dissolved at 10 mM DMS stock solution. 45 uL of stock solution was transferred to a 384 pp-plate. A 3-fold, 10-point dilution was performed via transferring 15 uL compound into 30 uL DMSO by using TECAN liquid handler.
2. The plates were spinned at room temperature at 1,000 RPM for 1 minute.
3. 50 nL of diluted compound was transferred from compound source plate into the cell plate by Echo.
4. After compound treatment for 6 hours, the plate was removed from incubators and equilibrated at room temperature for 15 minutes. The HiBit reagent was prepared, consisting of the following ratios: 10 mL of Nano-Glo®-HiBiT Lytic Buffer, 200 μL of Nano-Glo®-HiBiT Lytic Substrate and 100 uL of LgBiT Protein, mixed thoroughly. 20 μL of HiBit reagent was added into each well and the plates were spinned at room temperature at 1,000 RPM for 1 minute to be detected. Then the plates were shaken at 600 RPM at room temperature for 20 minutes, and read by EnVision.
5. The inhibition activity was calculated following the formula below:

$$\% Degrader = 100 \times ( LumHC - LumSample ) / ( LumHC - LumLC )$$

Note: Where HC is obtained from cells treated with 0.100 DMSO only; LC is obtained from culture medium only.
6. ABS DC₅₀ was calculated by fitting the Curve using Xlfit (v5.3.1.3), equation 201:

$$Y = Bottom + ( Top - Bottom ) / ( 1 + {10}^{∧} ( ( Log IC 50 - X ) * HillSlope ) )$$

The data from this assay is presented in Table 1, columns 3 and 4.

### HiBit Assay Protocol for CCNE1

### 3. Materials

3.1 Reagents

| Reagents | Vendor | Cat# |
|---|---|---|
| HEK293-CCNE1-Hibit cell | Pharmaron | \ |
| EMEM | ATCC | 30-2003 |
| FBS | Gibco | 10099141C |
| Nano-Glo ® HiBiT Lytic Detection | Promega | N3040 |
| System | | |
| 0.25% Trypsin/EDTA | Invitrogen | #25300 |
| PBS | Solarbio | P1020-500 |
| Penicillin-Streptomycin Liquid | Solarbio | P1400 |

1.2 Instruments

| Instrument | Vendor | Cat# |
|---|---|---|
| Centrifuge | Eppendorf | 5810R |
| CO₂ Incubator | Thermo | Model: 371 |
| Vortex | IKA | MS3 digital |
| Microplate shaker | Yoning | WZ-4 |
| Echo Liquid Handler | Labcyte | 550 |
| TECAN | TECAN | Freedom EVO200 |
| 384 well plate | Corning | 3570 |
| 384 pp-plate | Labcyte | 001-14555 |
| 50 mL centrifuge tube | BD-Falcon | 352098 |
| 15 mL centrifuge tube | BD-Falcon | 352097 |
| EnVision | PerkinElmer | 2105-0020 |

### 4. Assay Procedure

### Day 1.

1. The culture medium for HEK293-CCNE1-Hibit cell was prepared using EMEM with 1000 FBS.
2. Cells were cultivated in T-75 flasks in a cell culture incubator set at 37° C., 5% CO2, 95% relative humidity. Cells were allowed to reach 80-90% confluence before detaching and splitting.
3. Cultivated cells were rinsed in T-75 flasks with 5 mL PBS. Aspirate off Added was 1.5 mL trypsin, and the cells were incubated at 37° C. for approximately 5 minutes or until the cells detached and floated.
4. Trypsin was inactivated by adding excess serum containing medium.
5. Cells were harvested from flask into cell culture medium and then the cell number was counted. HEK293-CCNE1-Hibit cell were seeded into 384-well plate (cell density: 1.6×10⁵ cells/well/50 uL medium) in EMEM medium according to the plate map below and incubated overnight at 37° C. and 500 CO₂.
Note: Cell Cultured Medium: 90% EMEM+10% FBS+1% Penicillin-Streptomycin Liquid

Plate map

| Plate | | | | | | | | | | | | | |
|---|---|---|---|---|---|---|---|---|---|---|---|---|---|
| map | | | | | | | | | | | | | |
| Top(nM) | | 1 | 2 | 3 | 4 | 5 | 6 | 7 | 8 | 9 | 10 | 11 | 12 |
| | A | | | | | | | | | | | | |
| 10000 | B | NC | 10000 | 3333 | 1111 | 370 | 123 | 41.2 | 13.7 | 4.6 | 1.5 | 0.51 | PC |
| 10000 | C | NC | 10000 | 3333 | 1111 | 370 | 123 | 41.2 | 13.7 | 4.6 | 1.5 | 0.51 | PC |
| 10000 | D | NC | 10000 | 3333 | 1111 | 370 | 123 | 41.2 | 13.7 | 4.6 | 1.5 | 0.51 | PC |
| 10000 | E | NC | 10000 | 3333 | 1111 | 370 | 123 | 41.2 | 13.7 | 4.6 | 1.5 | 0.51 | PC |
| 10000 | F | NC | 10000 | 3333 | 1111 | 370 | 123 | 41.2 | 13.7 | 4.6 | 1.5 | 0.51 | PC |
| 10000 | G | NC | 10000 | 3333 | 1111 | 370 | 123 | 41.2 | 13.7 | 4.6 | 1.5 | 0.51 | PC |
| 10000 | H | NC | 10000 | 3333 | 1111 | 370 | 123 | 41.2 | 13.7 | 4.6 | 1.5 | 0.51 | PC |
| 10000 | I | NC | 10000 | 3333 | 1111 | 370 | 123 | 41.2 | 13.7 | 4.6 | 1.5 | 0.51 | PC |
| 10000 | J | NC | 10000 | 3333 | 1111 | 370 | 123 | 41.2 | 13.7 | 4.6 | 1.5 | 0.51 | PC |
| 10000 | K | NC | 10000 | 3333 | 1111 | 370 | 123 | 41.2 | 13.7 | 4.6 | 1.5 | 0.51 | PC |
| 10000 | L | NC | 10000 | 3333 | 1111 | 370 | 123 | 41.2 | 13.7 | 4.6 | 1.5 | 0.51 | PC |
| 10000 | M | NC | 10000 | 3333 | 1111 | 370 | 123 | 41.2 | 13.7 | 4.6 | 1.5 | 0.51 | PC |
| 10000 | N | NC | 10000 | 3333 | 1111 | 370 | 123 | 41.2 | 13.7 | 4.6 | 1.5 | 0.51 | PC |
| 10000 | O | NC | 10000 | 3333 | 1111 | 370 | 123 | 41.2 | 13.7 | 4.6 | 1.5 | 0.51 | PC |
| | P | | | | | | | | | | | | |
| Plate | | | | | | | | | | | | | |
| map | | | | | | | | | | | | | |
| Top(nM) | | 13 | 14 | 15 | 16 | 17 | 18 | 19 | 20 | 21 | 22 | 23 | 24 |
| | A | | | | | | | | | | | | |
| 10000 | B | PC | 10000 | 3333 | 1111 | 370 | 123 | 41.2 | 13.7 | 4.6 | 1.5 | 0.51 | NC |
| 10000 | C | PC | 10000 | 3333 | 1111 | 370 | 123 | 41.2 | 13.7 | 4.6 | 1.5 | 0.51 | NC |
| 10000 | D | PC | 10000 | 3333 | 1111 | 370 | 123 | 41.2 | 13.7 | 4.6 | 1.5 | 0.51 | NC |
| 10000 | E | PC | 10000 | 3333 | 1111 | 370 | 123 | 41.2 | 13.7 | 4.6 | 1.5 | 0.51 | NC |
| 10000 | F | PC | 10000 | 3333 | 1111 | 370 | 123 | 41.2 | 13.7 | 4.6 | 1.5 | 0.51 | NC |
| 10000 | G | PC | 10000 | 3333 | 1111 | 370 | 123 | 41.2 | 13.7 | 4.6 | 1.5 | 0.51 | NC |
| 10000 | H | PC | 10000 | 3333 | 1111 | 370 | 123 | 41.2 | 13.7 | 4.6 | 1.5 | 0.51 | NC |
| 10000 | I | PC | 10000 | 3333 | 1111 | 370 | 123 | 41.2 | 13.7 | 4.6 | 1.5 | 0.51 | NC |
| 10000 | J | PC | 10000 | 3333 | 1111 | 370 | 123 | 41.2 | 13.7 | 4.6 | 1.5 | 0.51 | NC |
| 10000 | K | PC | 10000 | 3333 | 1111 | 370 | 123 | 41.2 | 13.7 | 4.6 | 1.5 | 0.51 | NC |
| 10000 | L | PC | 10000 | 3333 | 1111 | 370 | 123 | 41.2 | 13.7 | 4.6 | 1.5 | 0.51 | NC |
| 10000 | M | PC | 10000 | 3333 | 1111 | 370 | 123 | 41.2 | 13.7 | 4.6 | 1.5 | 0.51 | NC |
| 10000 | N | PC | 10000 | 3333 | 1111 | 370 | 123 | 41.2 | 13.7 | 4.6 | 1.5 | 0.51 | NC |
| 10000 | O | PC | 10000 | 3333 | 1111 | 370 | 123 | 41.2 | 13.7 | 4.6 | 1.5 | 0.51 | NC |
| | P | | | | | | | | | | | | |

### Day 2.

1. Test compounds were dissolved at 10 mM DMSO stock solution. 45 μL of stock solution was transferred to a 384 pp-plate. A 3-fold, 10-point dilution was performed via transferring 15 uL compound into 30 uL DMSO by using TECAN liquid handler.
2. The plates were spinned at room temperature at 1,000 RPM for 1 minute.
3. 50 nL of diluted compound was transferred from compound source plate into the cell plate by Echo.
4. After compound treatment for 6 hours, the plate was removed from incubators and equilibrated at room temperature for 15 minutes. The HiBit reagent was prepared, consisting of the following ratios: 10 mL of Nano-Glo®-HiBiT Lytic Buffer, 200 μL of Nano-Glo®-HiBiT Lytic Substrate and 100 μL of LgBiT Protein, mix thoroughly. 20 μL of HiBit reagent was added into each well and the plates were spinned at room temperature at 1,000 RPM for 1 minute to be detected. Then the plates were shaken at 600 RPM at room temperature for 20 minutes, and read by EnVision.
5. The inhibition activity was calculated following the formula below:

$$\% Degrader = 100 \times ( LumHC - LumSample ) / ( LumHC - LumLC )$$

Note: Where HC is obtained from cells treated with 0.100 DMSO only; LC is obtained from culture medium only.
6. ABS DC₅₀ was calculated by fitting the Curve using Xlfit (v5.3.1.3), equation 201:

$$Y = Bottom + ( Top - Bottom ) / ( 1 + {10}^{∧} ( ( Log IC 50 - X ) * HillSlope ) )$$

The data from this assay is presented in Table 1, columns 5 and 6.

### HiBit Assay Protocol for CDK9

### 1. Materials

1.1 Reagents

| Reagents | Vendor | Cat# |
|---|---|---|
| HEK293-CDK9-Hibit cell | Pharmaron | \ |
| DMEM | Hyclone | SH30022 01 |
| FBS | Gibco | 10099141C |
| Nano-Glo ® HiBiT Lytic | Promega | N3040 |
| Detection System | | |
| 0.25% Trypsin/EDTA | Invitrogen | #25300 |
| PBS | Solarbio | P1020-500 |
| Penicillin-Streptomycin Liquid | Solarbio | P1400 |

1.2Instruments

| Instrument | Vendor | Cat# |
|---|---|---|
| Centrifuge | Eppendorf | 5810R |
| CO₂ Incubator | Thermo | Model: 371 |
| Vortex | IKA | MS3 digital |
| Microplate shaker | Yoning | WZ-4 |
| Echo Liquid Handler | Labcyte | 550 |
| TECAN | TECAN | Freedom EVO200 |
| 384 well plate | Corning | 3570 |
| 384 pp-plate | Labcyte | 001-14555 |
| 50 mL centrifuge tube | BD-Falcon | 352098 |
| 15 mL centrifuge tube | BD-Falcon | 352097 |
| EnVision | PerkinElmer | 2105-0020 |

### 2. Assay Procedure

### Day 1.

1. The culture medium for HEK293-CDK9-Hibit cell was prepared using DMEM with 10% FBS.
2. Cells were cultivated in T-75 flasks in a cell culture incubator set at 37° C., 5% CO2, 95% relative humidity. Cells were allowed to reach 80-90% confluence before detaching and splitting.
3. Cultivated cells were rinsed in T-75 flasks with 5 mL PBS. Aspirated off. Added 1.5 mL trypsin, and incubated the cells at 37° C. for approximately 5 minutes or until the cells detached and floated.
4. Trypsin was inactivated by adding excess serum containing medium.
5. Cells were harvested from flask into cell culture medium and then the cell number was counted. HEK293-CDK9-Hibit cells were seeded into 384-well plate (cell density: 1.6×105 cells/well/50 uL medium) in DMEM medium according to the plate map below and were incubated overnight at 37° C. and 5% CO2.
Note: Cell Cultured Medium: 90% DMEM+10% FBS+1% Penicillin-Streptomycin Liquid

FIG. 1 Plate map

| Plate | | | | | | | | | | | | | |
|---|---|---|---|---|---|---|---|---|---|---|---|---|---|
| map | | | | | | | | | | | | | |
| Top(nM) | | 1 | 2 | 3 | 4 | 5 | 6 | 7 | 8 | 9 | 10 | 11 | 12 |
| | A | | | | | | | | | | | | |
| 10000 | B | NC | 10000 | 3333 | 1111 | 370 | 123 | 41.2 | 13.7 | 4.6 | 1.5 | 0.51 | PC |
| 10000 | C | NC | 10000 | 3333 | 1111 | 370 | 123 | 41.2 | 13.7 | 4.6 | 1.5 | 0.51 | PC |
| 10000 | D | NC | 10000 | 3333 | 1111 | 370 | 123 | 41.2 | 13.7 | 4.6 | 1.5 | 0.51 | PC |
| 10000 | E | NC | 10000 | 3333 | 1111 | 370 | 123 | 41.2 | 13.7 | 4.6 | 1.5 | 0.51 | PC |
| 10000 | F | NC | 10000 | 3333 | 1111 | 370 | 123 | 41.2 | 13.7 | 4.6 | 1.5 | 0.51 | PC |
| 10000 | G | NC | 10000 | 3333 | 1111 | 370 | 123 | 41.2 | 13.7 | 4.6 | 1.5 | 0.51 | PC |
| 10000 | H | NC | 10000 | 3333 | 1111 | 370 | 123 | 41.2 | 13.7 | 4.6 | 1.5 | 0.51 | PC |
| 10000 | I | NC | 10000 | 3333 | 1111 | 370 | 123 | 41.2 | 13.7 | 4.6 | 1.5 | 0.51 | PC |
| 10000 | J | NC | 10000 | 3333 | 1111 | 370 | 123 | 41.2 | 13.7 | 4.6 | 1.5 | 0.51 | PC |
| 10000 | K | NC | 10000 | 3333 | 1111 | 370 | 123 | 41.2 | 13.7 | 4.6 | 1.5 | 0.51 | PC |
| 10000 | L | NC | 10000 | 3333 | 1111 | 370 | 123 | 41.2 | 13.7 | 4.6 | 1.5 | 0.51 | PC |
| 10000 | M | NC | 10000 | 3333 | 1111 | 370 | 123 | 41.2 | 13.7 | 4.6 | 1.5 | 0.51 | PC |
| 10000 | N | NC | 10000 | 3333 | 1111 | 370 | 123 | 41.2 | 13.7 | 4.6 | 1.5 | 0.51 | PC |
| 10000 | O | NC | 10000 | 3333 | 1111 | 370 | 123 | 41.2 | 13.7 | 4.6 | 1.5 | 0.51 | PC |
| | P | | | | | | | | | | | | |
| Plate | | | | | | | | | | | | | |
| map | | | | | | | | | | | | | |
| Top(nM) | | 13 | 14 | 15 | 16 | 17 | 18 | 19 | 20 | 21 | 22 | 23 | 24 |
| | A | | | | | | | | | | | | |
| 10000 | B | PC | 10000 | 3333 | 1111 | 370 | 123 | 41.2 | 13.7 | 4.6 | 1.5 | 0.51 | NC |
| 10000 | C | PC | 10000 | 3333 | 1111 | 370 | 123 | 41.2 | 13.7 | 4.6 | 1.5 | 0.51 | NC |
| 10000 | D | PC | 10000 | 3333 | 1111 | 370 | 123 | 41.2 | 13.7 | 4.6 | 1.5 | 0.51 | NC |
| 10000 | E | PC | 10000 | 3333 | 1111 | 370 | 123 | 41.2 | 13.7 | 4.6 | 1.5 | 0.51 | NC |
| 10000 | F | PC | 10000 | 3333 | 1111 | 370 | 123 | 41.2 | 13.7 | 4.6 | 1.5 | 0.51 | NC |
| 10000 | G | PC | 10000 | 3333 | 1111 | 370 | 123 | 41.2 | 13.7 | 4.6 | 1.5 | 0.51 | NC |
| 10000 | H | PC | 10000 | 3333 | 1111 | 370 | 123 | 41.2 | 13.7 | 4.6 | 1.5 | 0.51 | NC |
| 10000 | I | PC | 10000 | 3333 | 1111 | 370 | 123 | 41.2 | 13.7 | 4.6 | 1.5 | 0.51 | NC |
| 10000 | J | PC | 10000 | 3333 | 1111 | 370 | 123 | 41.2 | 13.7 | 4.6 | 1.5 | 0.51 | NC |
| 10000 | K | PC | 10000 | 3333 | 1111 | 370 | 123 | 41.2 | 13.7 | 4.6 | 1.5 | 0.51 | NC |
| 10000 | L | PC | 10000 | 3333 | 1111 | 370 | 123 | 41.2 | 13.7 | 4.6 | 1.5 | 0.51 | NC |
| 10000 | M | PC | 10000 | 3333 | 1111 | 370 | 123 | 41.2 | 13.7 | 4.6 | 1.5 | 0.51 | NC |
| 10000 | N | PC | 10000 | 3333 | 1111 | 370 | 123 | 41.2 | 13.7 | 4.6 | 1.5 | 0.51 | NC |
| 10000 | O | PC | 10000 | 3333 | 1111 | 370 | 123 | 41.2 | 13.7 | 4.6 | 1.5 | 0.51 | NC |
| | P | | | | | | | | | | | | |

### Day 2.

1. Test compounds were dissolved at 3 mM DMSO stock solution. Transferred 45 uL of stock solution to a 384 pp-plate. Perform 3-fold, 10-point dilution via transferring 15 uL compound into 30 uL DMSO by using TECAN liquid handler.
2. The plates were spinned at room temperature at 1,000 RPM for 1 minute.
3. Transferred 50 nL of diluted compound from compound source plate into the cell plate by Echo.
4. After compound treatment for 6 hours, removed the plate from incubators and equilibrated at room temperature for 15 minutes. Prepared the HiBit reagent consists of the following ratios: 10 mL of Nano-Glo®-HiBiT Lytic Buffer, 200 uL of Nano-Glo®-HiBiT Lytic Substrate and 100 uL of LgBiT Protein, mixed thoroughly. Added 20 uL of HiBit reagent into each well and spinned the plates at room temperature at 1,000 RPM for 1 minute to be detected. Plates were then shaken at 600 RPM at room temperature for 20 minutes, and read by EnVision.
5. The inhibition activity was calculated following the formula below:

$$\% Degrader = 100 \times ( LumHC - LumSample ) / ( LumHC - LumLC )$$

Note: Where HC was obtained from cells treated with 0.1% DMSO only; LC was obtained from culture medium only.
6. The ABS DC50 was calculated by fitting the Curve using Xlfit (v5.3.1.3), equation 201:

$$Y = Bottom + ( Top - Bottom ) / ( 1 + 10 \hat{} ( ( Log IC 50 - X ) * HillSlope ) )$$

The data from this assay is presented in Table 1, columns 7 and 8.

1. A compound of Formula A: 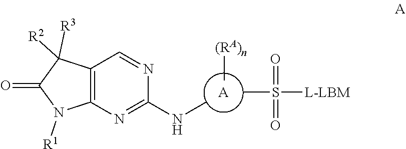
or a pharmaceutically acceptable salt thereof, wherein:
Ring A is phenyl, a 3-14 membered cycloalkyl or 4-14 membered heterocyclyl containing 1-3 heteroatoms independently selected from N, O and S;
R¹ is selected from —C₁₋₆ alkyl, —C₂₋₆ alkenyl, —C₂₋₆ alkynyl, —C₁₋₆ haloalkyl, —C₁₋₆ heteroalkyl, —C₃₋₁₄ cycloalkyl, 6-14 membered aryl, 4-14 membered heterocyclyl, 5-14 membered heteroaryl, —C₁₋₄ alkyl-C₃₋₁₄ cycloalkyl, —C₁₋₄ alkyl-(6-14 membered aryl), —C₁₋₄ alkyl-(4-14 membered heterocyclyl), and —C₁₋₄ alkyl (5-14 membered heteroaryl), wherein each heterocyclyl and heteroaryl contains 1-3 heteroatoms independently selected from N, O and S, and wherein each alkyl, alkenyl, alkynyl, cycloalkyl, aryl, heterocyclyl, and heteroaryl is substituted with 0, 1, 2, 3 or 4 instances of R⁶;
each R² and R³ is independently selected from —C₁₋₆ alkyl, —C₁₋₆ haloalkyl, —C₁₋₆ heteroalkyl, —C₂₋₆ alkenyl, —C₂₋₆ alkynyl, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, and wherein each alkyl, alkenyl, alkynyl, cycloalkyl, aryl, heterocyclyl, and heteroaryl is substituted with 0, 1, 2, 3 or 4 instances of R^{B};
or R² and R³, together with the carbon atom to which they are attached, form Ring B, wherein Ring B is a 3-7 membered cycloalkyl ring or a 4-7 membered heterocyclyl ring containing 1 or 3 heteroatoms independently selected from N, O and S, wherein Ring B is substituted with 0, 1, 2, 3, or 4 instances of R^{B};
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(═O)—, —C(═O)O—, —C(═O)—, —S(═O)—, —S(═O)₂—, —NRS(═O)₂—, —S(═O)₂NR—, —NRC(═O)—, —C(═O)NR—, —OC(═O)NR— or —NRC(═O)O—, wherein:
each -Cy- is independently a bivalent ring selected from phenylene, an 8-10 membered bicyclic arylene, a 4-7 membered monocyclic carbocyclylene, a 5-11 membered spiro carbocyclylene, a 4-10 membered bicyclic carbocyclylene, a 5-10 membered bridged carbocyclylene, a 4-7 membered monocyclic heterocyclylene having 1-2 heteroatoms independently selected from nitrogen, oxygen, and sulfur, a 5-11 membered spiro heterocyclylene having 1-2 heteroatoms independently selected from nitrogen, oxygen, and sulfur, an 4-10 membered bicyclic heterocyclylene having 1-2 heteroatoms independently selected from nitrogen, oxygen, and sulfur, a 5-10 membered bridged bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur, wherein each phenylene, arylene, carbocyclylene, heterocyclylene and heteroarylene is substituted with 0, 1, 2, 3, or 4 instances of R^{C};
LBM is selected from 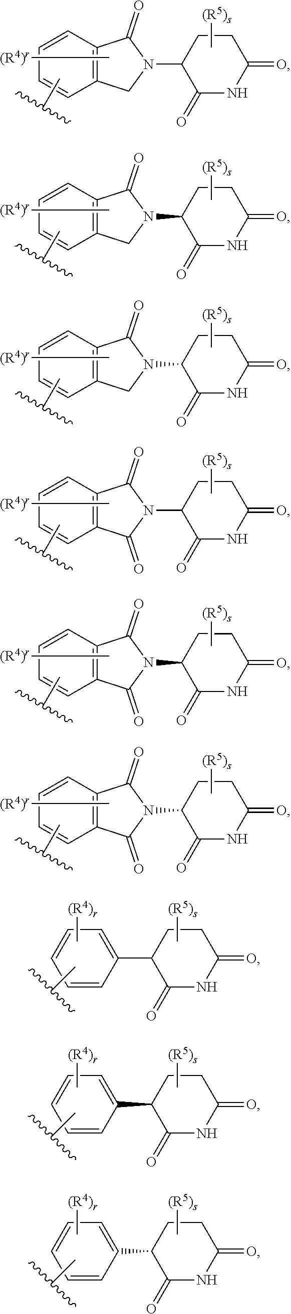 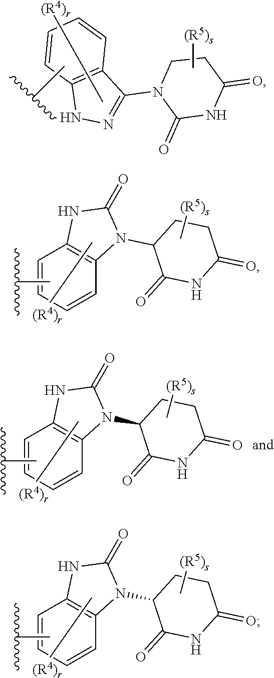
each instance of R^{A}, R^{B}, R^{C}, R⁴, R⁵ and R⁶ is independently selected from oxo, deuterium, halogen, —C₁₋₆ alkyl, —C₁₋₆ heteroalkyl, —C₁₋₆ haloalkyl, —C₂₋₆ alkenyl, —C₂₋₆ alkynyl, —CN, —NO₂, —OR, —SR, —NR₂, —S(O)₂R, —S(O)₂NR₂, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S;
each instance of R is independently hydrogen, —C₁₋₆ alkyl, —C₁₋₆ haloalkyl, —C₁₋₆ heteroalkyl, —C₂₋₆ alkenyl, —C₂₋₆ alkynyl, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, or two R groups attached to the same nitrogen are optionally taken together with nitrogen to which they are attached to form an optionally substituted 4-7 heterocyclyl having 0, 1 or 2 additional heteroatoms independently selected from N, O and S;
n is 0, 1, 2, 3, or 4;
r is 0, 1, 2, 3, or 4; and
s is 0, 1, 2, 3, or 4.

2. The compound of claim 1, or a pharmaceutically acceptable salt thereof, wherein the compound is of Formula B: 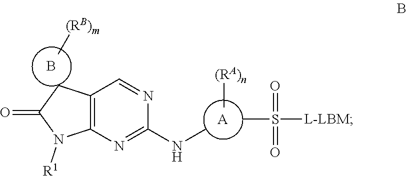
wherein Ring B is a 3-7 membered cycloalkyl ring or a 4-7 membered heterocyclyl ring containing 1 or 3 heteroatoms independently selected from N, O and S.

3. A compound of Formula I or Formula I-1: 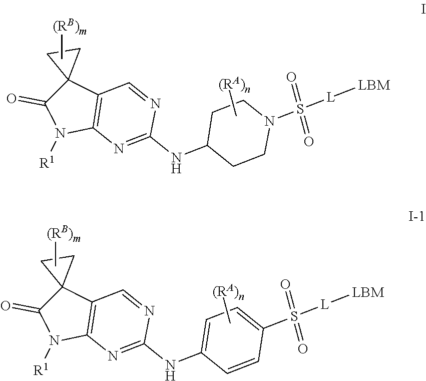
or a pharmaceutically acceptable salt thereof, wherein:
R¹ is selected from —C₁₋₆ alkyl, —C₂₋₆ alkenyl, —C₂₋₆ alkynyl, —C₁₋₆ haloalkyl, —C₁₋₆ heteroalkyl, —C₃₋₁₄ cycloalkyl, 6-14 membered aryl, 4-14 membered heterocyclyl, 5-14 membered heteroaryl, —C₁₋₄ alkyl-C₃₋₁₄ cycloalkyl, —C₁₋₄ alkyl-(6-14 membered aryl), —C₁₋₄ alkyl-(4-14 membered heterocyclyl), and —C₁₋₄ alkyl (5-14 membered heteroaryl), wherein each heterocyclyl and heteroaryl contains 1-3 heteroatoms independently selected from N, O and S, and wherein each alkyl, alkenyl, alkynyl, cycloalkyl, aryl, heterocyclyl, and heteroaryl is substituted with 0, 1, 2, 3 or 4 instances of R⁶;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(═O)—, —C(═O)O—, —C(═O)—, —S(═O)—, —S(═O)₂—, —NRS(═O)₂—, —S(═O)₂NR—, —NRC(═O)—, —C(═O)NR—, —OC(═O)NR— or —NRC(═O)O—, wherein:
each -Cy- is independently a bivalent ring selected from phenylene, an 8-10 membered bicyclic arylene, a 4-7 membered monocyclic carbocyclylene, a 5-11 membered spiro carbocyclylene, a 4-10 membered bicyclic carbocyclylene, a 5-10 membered bridged carbocyclylene, a 4-7 membered monocyclic heterocyclylene having 1-2 heteroatoms independently selected from nitrogen, oxygen, and sulfur, a 5-11 membered spiro heterocyclylene having 1-2 heteroatoms independently selected from nitrogen, oxygen, and sulfur, an 4-10 membered bicyclic heterocyclylene having 1-2 heteroatoms independently selected from nitrogen, oxygen, and sulfur, a 5-10 membered bridged bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur, wherein each phenylene, arylene, carbocyclylene, heterocyclylene and heteroarylene is substituted with 0, 1, 2, 3, or 4 instances of R^{C};
LBM is selected from 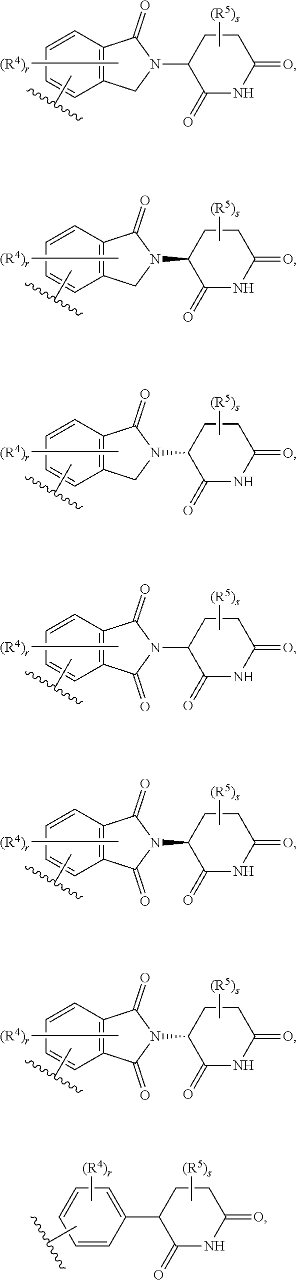 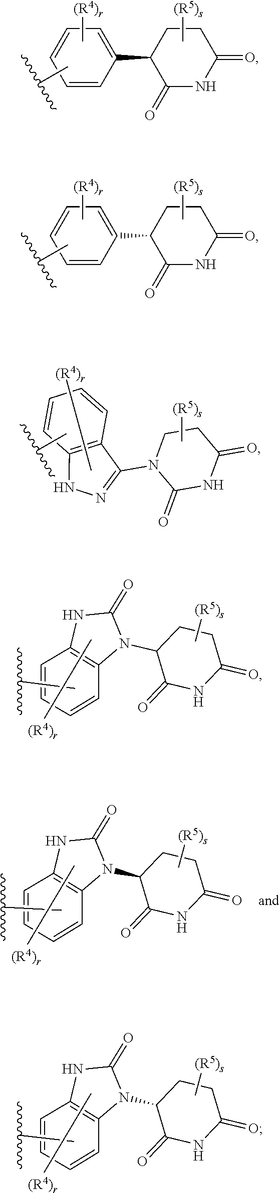
each instance of R^{A}, R^{B}, R^{C}, R⁴, R⁵ and R⁶ is independently selected from oxo, deuterium, halogen, —C₁₋₆ alkyl, —C₁₋₆ heteroalkyl, —C₁₋₆ haloalkyl, —C₂₋₆ alkenyl, —C₂₋₆ alkynyl, —CN, —NO₂, —OR, —SR, —NR₂, —S(O)₂R, —S(O)₂NR₂, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S;
each instance of R is independently hydrogen, —C₁₋₆ alkyl, —C₁₋₆ haloalkyl, —C₁₋₆ heteroalkyl, —C₂₋₆ alkenyl, —C₂₋₆ alkynyl, —C₃₋₇ cycloalkyl, phenyl, 4-7 membered heterocyclyl, and 5-6 membered heteroaryl, wherein said each heterocyclyl and heteroaryl contains 1-3 heteroatoms independently selected from N, O and S, or two R groups attached to the same nitrogen are optionally taken together with nitrogen to which they are attached to form an optionally substituted 4-7 heterocyclyl having 0, 1 or 2 additional heteroatoms independently selected from N, O and S;
m is 0, 1, 2, 3, or 4;
n is 0, 1, 2, 3, or 4;
r is 0, 1, 2, 3, or 4; and
s is 0, 1, 2, 3, or 4.

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. The compound of claim 3, or a pharmaceutically acceptable salt thereof, wherein the compound is of Formula II-A: 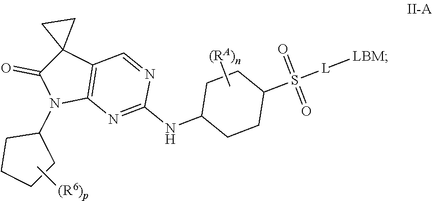
wherein p is 0, 1, 2, 3 or 4.

16. (canceled)

17. (canceled)

18. The compound of claim 3, or a pharmaceutically acceptable salt thereof, wherein the compound is of Formula II-1-B: 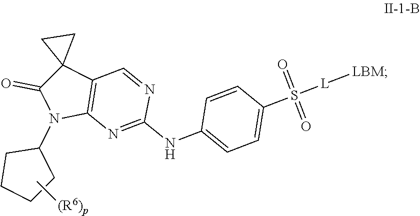
wherein p is 0, 1, 2, 3 or 4.

19. (canceled)

20. The compound of claim 1, or a pharmaceutically acceptable salt thereof, wherein the compound is of Formula K: 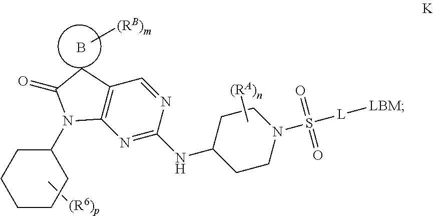
wherein p is 0, 1, 2, 3 or 4.

21. The compound of claim 3, or a pharmaceutically acceptable salt thereof, wherein the compound is of Formula IV-A: 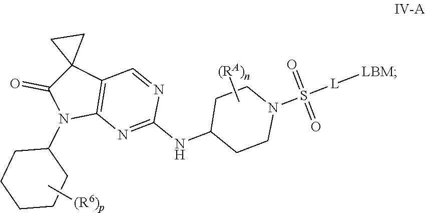
wherein p is 0, 1, 2, 3 or 4.

22. (canceled)

23. (canceled)

24. The compound of claim 3, or a pharmaceutically acceptable salt thereof, wherein the compound is of Formula IV-1-B: 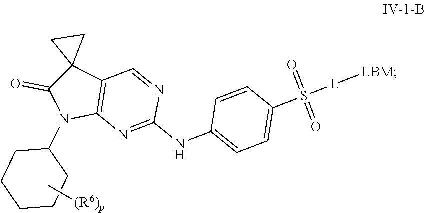
wherein p is 0, 1, 2, 3 or 4.

25. (canceled)

26. (canceled)

27. (canceled)

28. (canceled)

29. The compound of claim 1, or a pharmaceutically acceptable salt thereof, wherein R¹ is selected from 2-methylcyclopentyl 3-hydroxycyclohexyl,

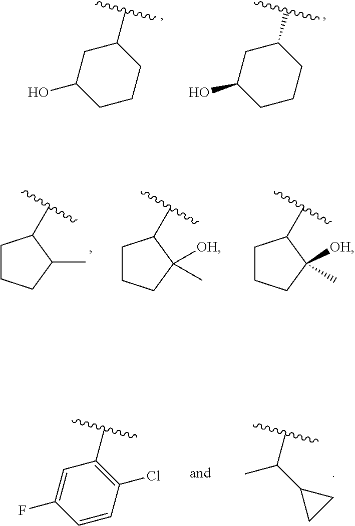

30. (canceled)

31. (canceled)

32. (canceled)

33. (canceled)

34. (canceled)

35. (canceled)

36. The compound of claim 1, or a pharmaceutically acceptable salt thereof, wherein the compound is of Formula M:

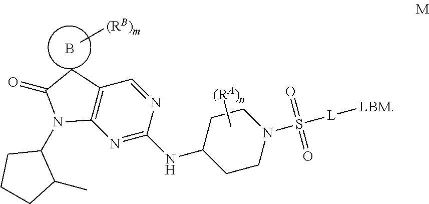

37. The compound of claim 3, or a pharmaceutically acceptable salt thereof, wherein the compound is of Formula III-A:

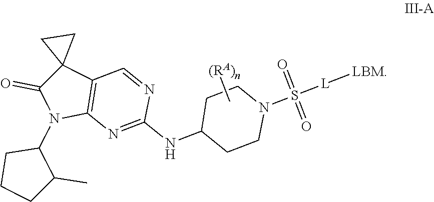

38. (canceled)

39. (canceled)

40. The compound of claim 3, or a pharmaceutically acceptable salt thereof, wherein the compound is of Formula III-1-B:

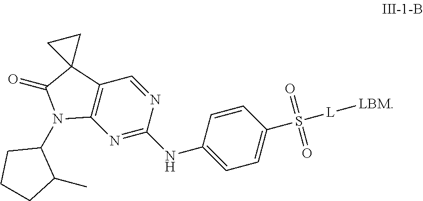

41. The compound of claim 3, or a pharmaceutically acceptable salt thereof, wherein the compound is of Formula V-D:

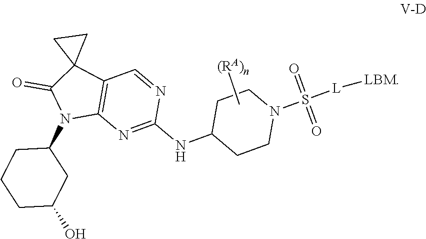

42. (canceled)

43. (canceled)

44. The compound of claim 3, or a pharmaceutically acceptable salt thereof, wherein the compound is of Formula V-1-E:

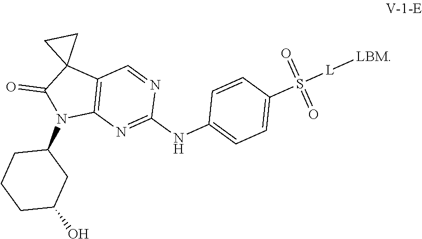

45. (canceled)

46. (canceled)

47. (canceled)

48. The compound of claim 1, or a pharmaceutically acceptable salt thereof, wherein the moiety represented by 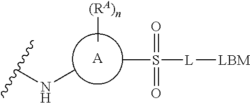
or by 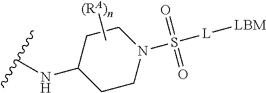
is selected from

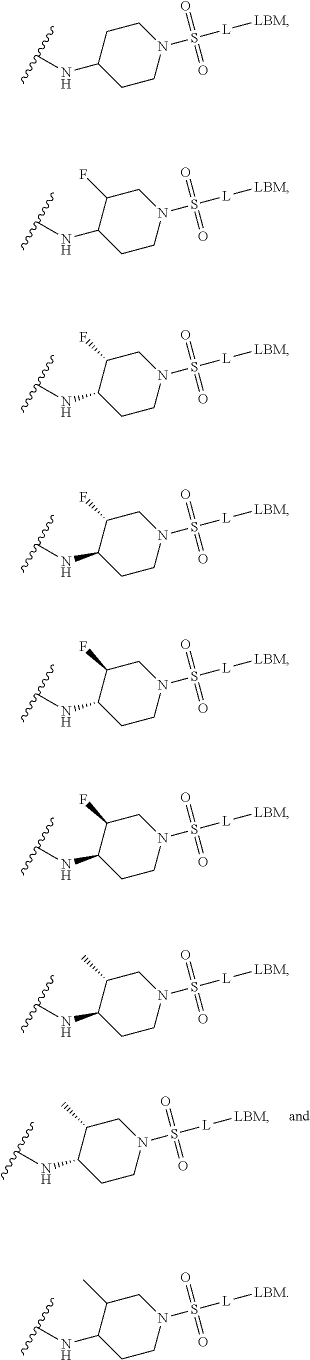

49. (canceled)

50. (canceled)

51. (canceled)

52. (canceled)

53. (canceled)

54. The compound of claim 1, or a pharmaceutically acceptable salt thereof, wherein L is selected from: 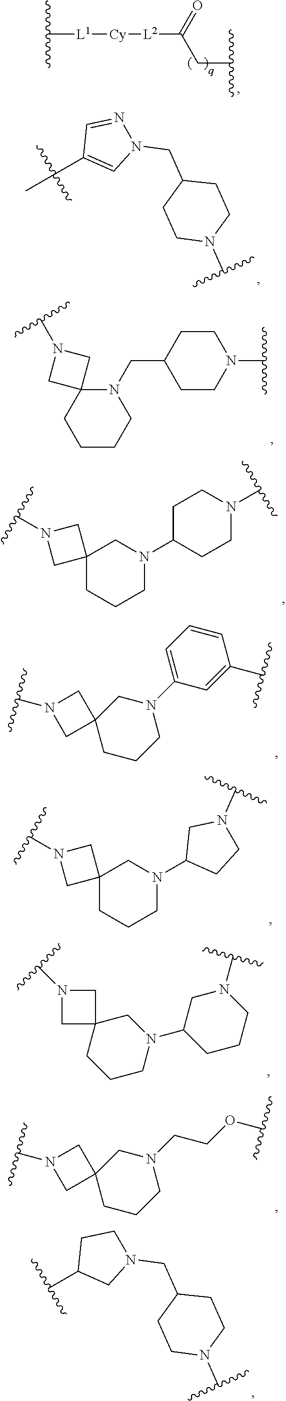 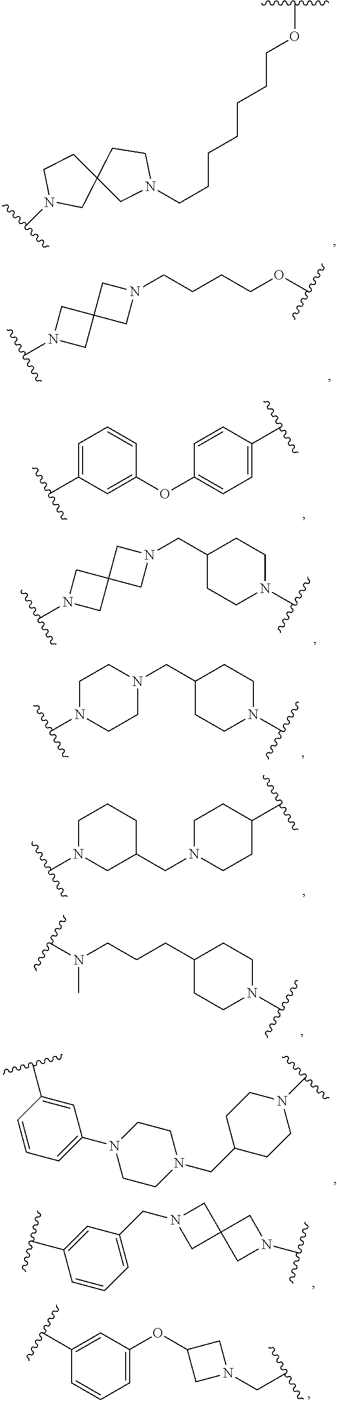 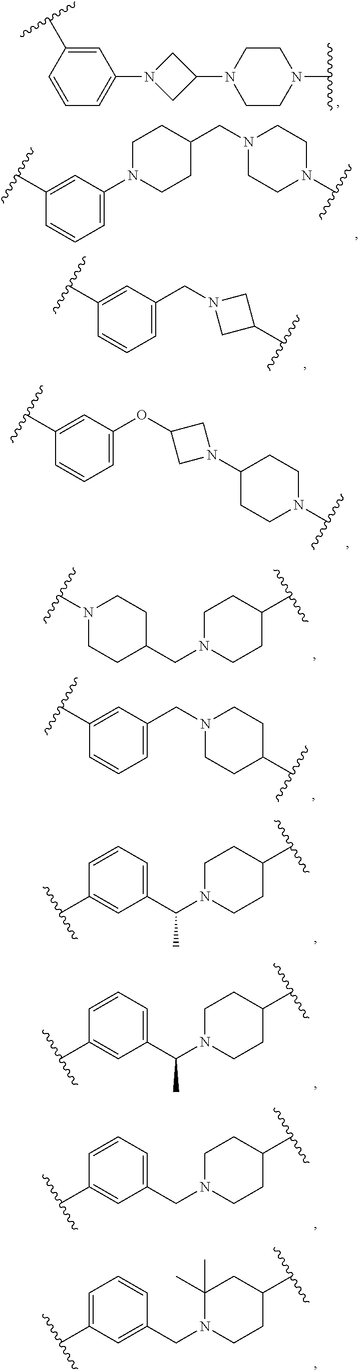 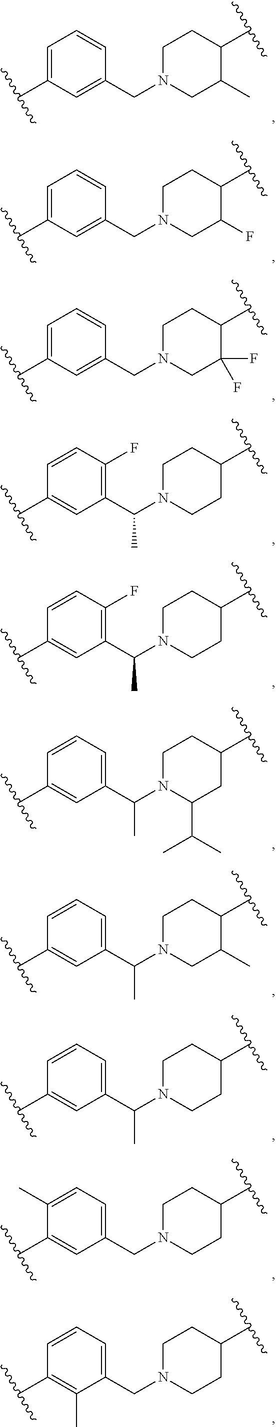 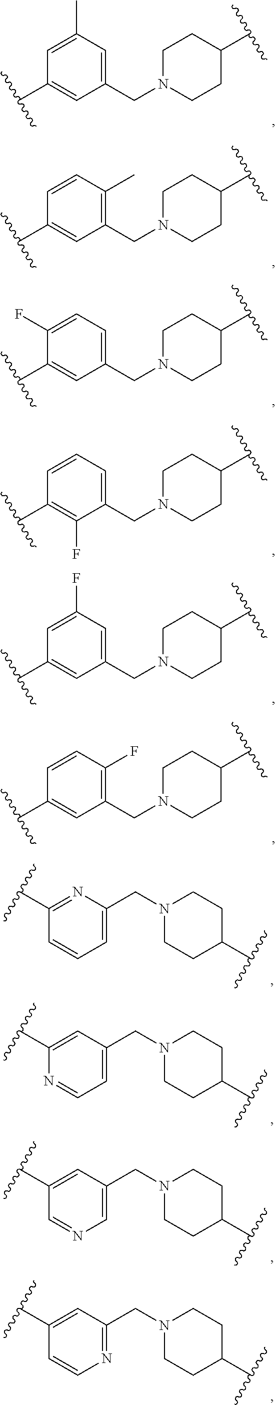 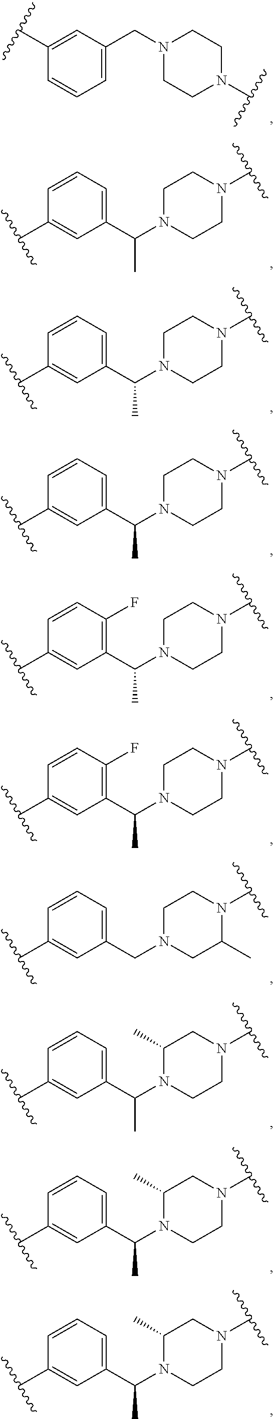 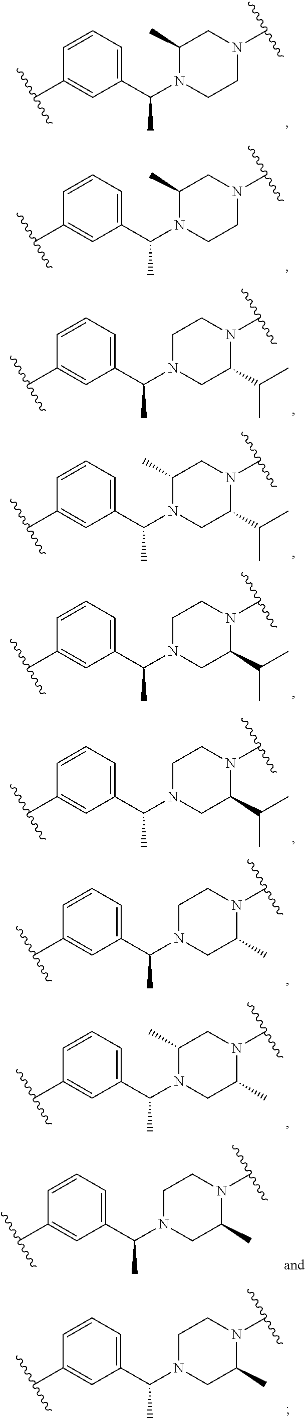
wherein the left side attachment point connects to the —S(O)₂— group and the right side attachment point connects to LBM;
L¹ and L² are each independently selected from a bond and —N(R′), wherein R′ is selected from H and C₁₋₆ alkyl; and
q is 0, 1, 2, 3, 4, 5, 6, 7, 8, 9 or 10.

55. (canceled)

56. (canceled)

57. The compound of claim 1, or a pharmaceutically acceptable salt thereof, wherein Cy is selected from: 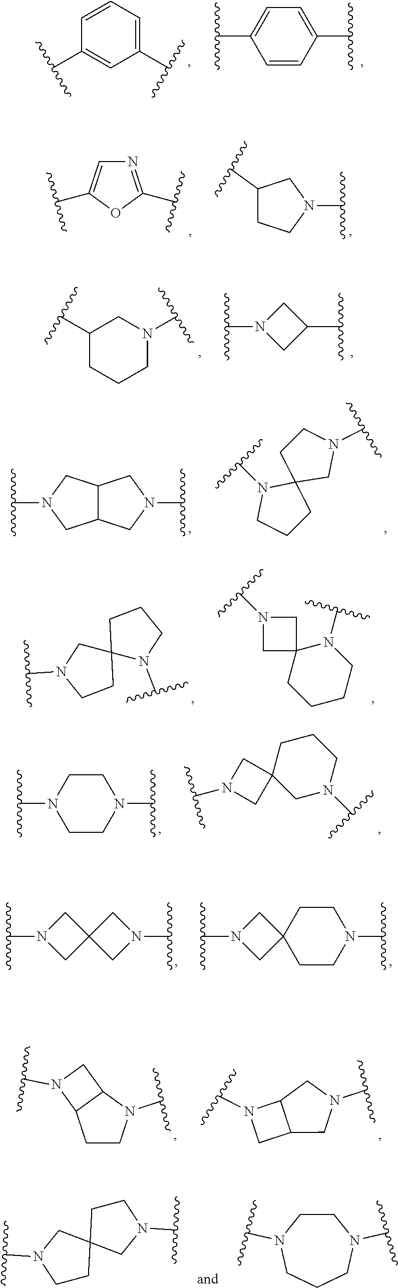
each not further substituted, wherein the left side attachment point connects to L¹ group and the right side attachment point connects to L².

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. The compound of claim 1, or a pharmaceutically acceptable salt thereof, wherein LBM is selected from

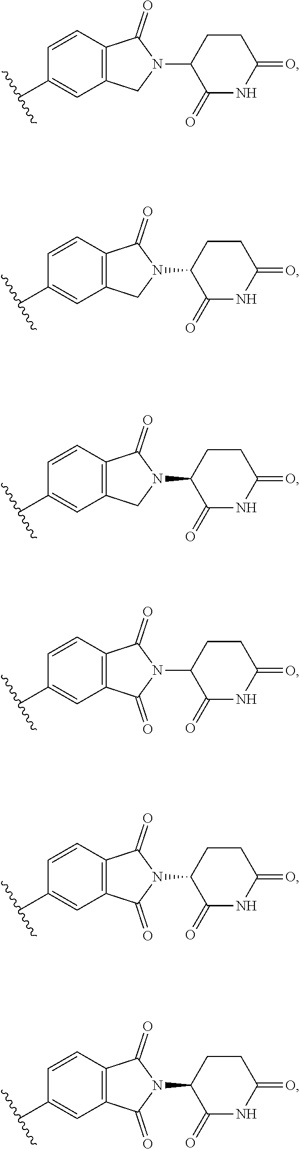 [SMILES: *c1ccc2c(c1)CN(C1CCC(=O)NC1=O)C2=O ; *c1ccc2c(c1)CN([C@@H]1CCC(=O)NC1=O)C2=O ; *c1ccc2c(c1)CN([C@H]1CCC(=O)NC1=O)C2=O ; *c1ccc2c(c1)C(=O)N(C1CCC(=O)NC1=O)C2=O ; *c1ccc2c(c1)C(=O)N([C@@H]1CCC(=O)NC1=O)C2=O ; *c1ccc2c(c1)C(=O)N([C@H]1CCC(=O)NC1=O)C2=O]

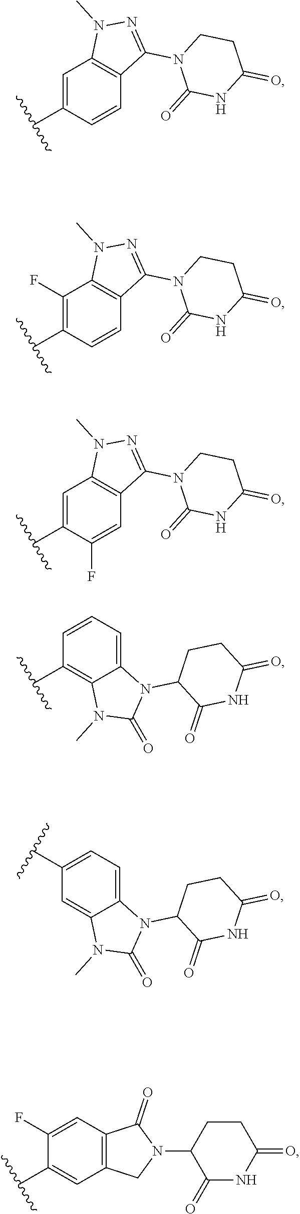

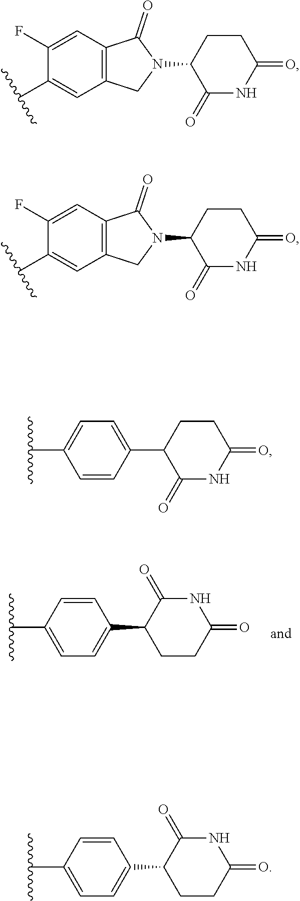 [SMILES: *c1cc2c(cc1F)C(=O)N([C@@H]1CCC(=O)NC1=O)C2 ; *c1cc2c(cc1F)C(=O)N([C@H]1CCC(=O)NC1=O)C2 ; *c1ccc(C2CCC(=O)NC2=O)cc1 ; *c1ccc([C@@H]2CCC(=O)NC2=O)cc1 ; *c1ccc([C@H]2CCC(=O)NC2=O)cc1]

74. (canceled)

75. The compound of claim 1, or a pharmaceutically acceptable salt thereof, wherein the compound is selected from

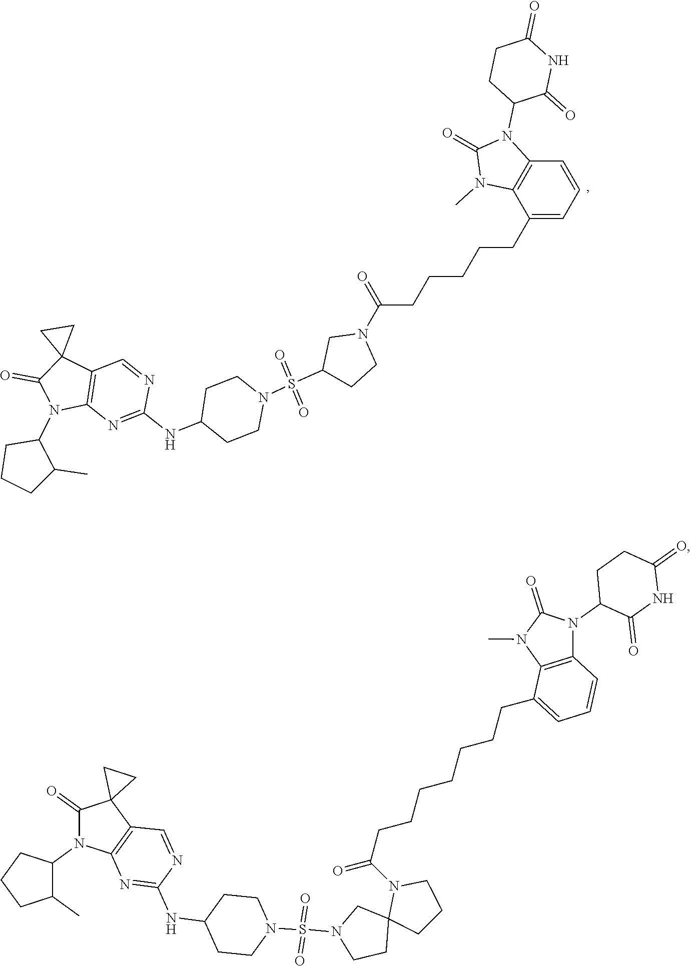 [SMILES: CC1CCCC1N1C(=O)C2(CC2)c2cnc(NC3CCN(S(=O)(=O)C4CCN(C(=O)CCCCCc5cccc6c5n(C)c(=O)n6C5CCC(=O)NC5=O)C4)CC3)nc21 ; CC1CCCC1N1C(=O)C2(CC2)c2cnc(NC3CCN(S(=O)(=O)N4CCC5(CCCN5C(=O)CCCCCCCc5cccc6c5n(C)c(=O)n6C5CCC(=O)NC5=O)C4)CC3)nc21]

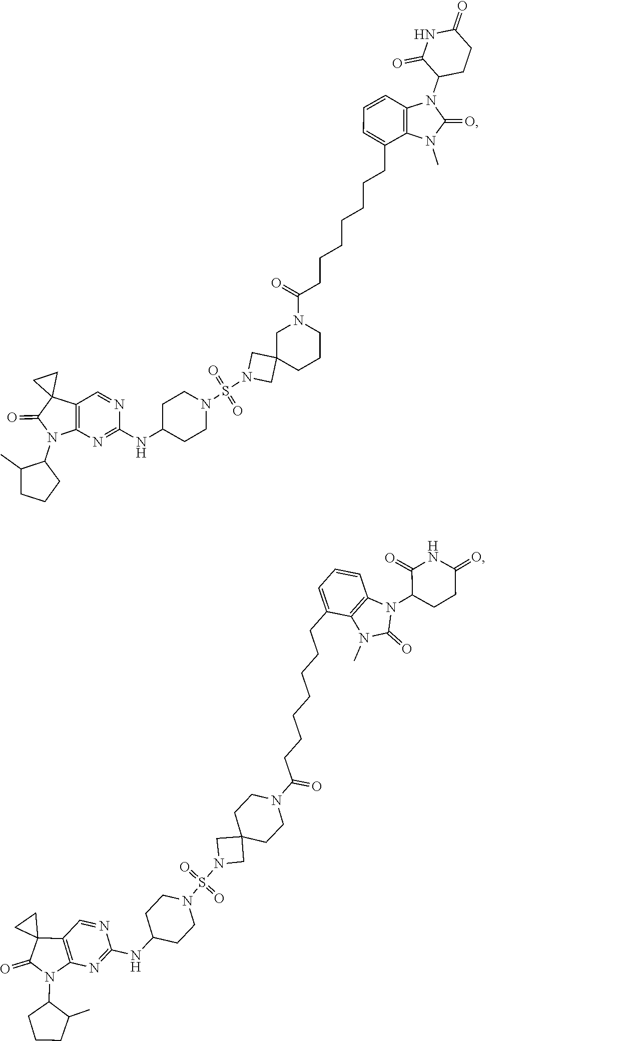 [SMILES: CC1CCCC1N1C(=O)C2(CC2)c2cnc(NC3CCN(S(=O)(=O)N4CC5(CCCN(C(=O)CCCCCCCc6cccc7c6n(C)c(=O)n7C6CCC(=O)NC6=O)C5)C4)CC3)nc21 ; CC1CCCC1N1C(=O)C2(CC2)c2cnc(NC3CCN(S(=O)(=O)N4CC5(CCN(C(=O)CCCCCCCc6cccc7c6n(C)c(=O)n7C6CCC(=O)NC6=O)CC5)C4)CC3)nc21]

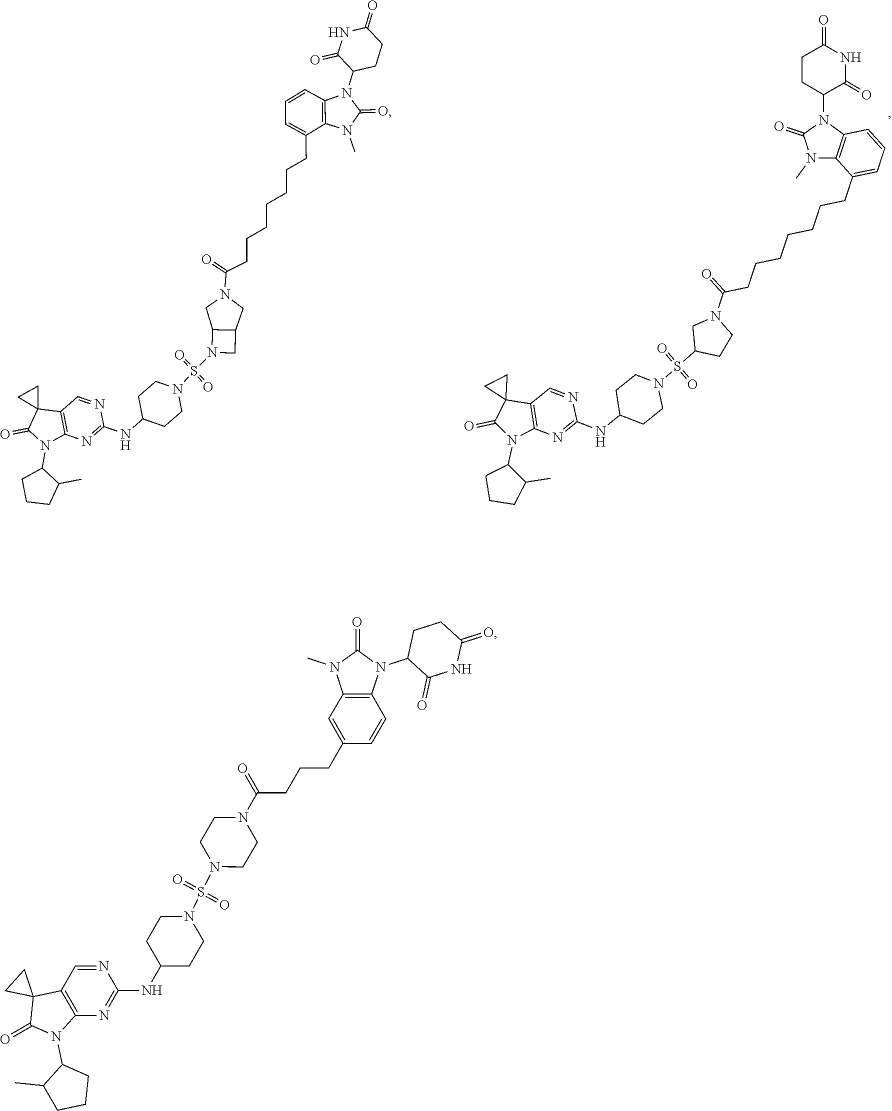 [SMILES: CC1CCCC1N1C(=O)C2(CC2)c2cnc(NC3CCN(S(=O)(=O)N4CC5CN(C(=O)CCCCCCCc6cccc7c6n(C)c(=O)n7C6CCC(=O)NC6=O)CC54)CC3)nc21 ; CC1CCCC1N1C(=O)C2(CC2)c2cnc(NC3CCN(S(=O)(=O)C4CCN(C(=O)CCCCCCCc5cccc6c5n(C)c(=O)n6C5CCC(=O)NC5=O)C4)CC3)nc21 ; CC1CCCC1N1C(=O)C2(CC2)c2cnc(NC3CCN(S(=O)(=O)N4CCN(C(=O)CCCc5ccc6c(c5)n(C)c(=O)n6C5CCC(=O)NC5=O)CC4)CC3)nc21]

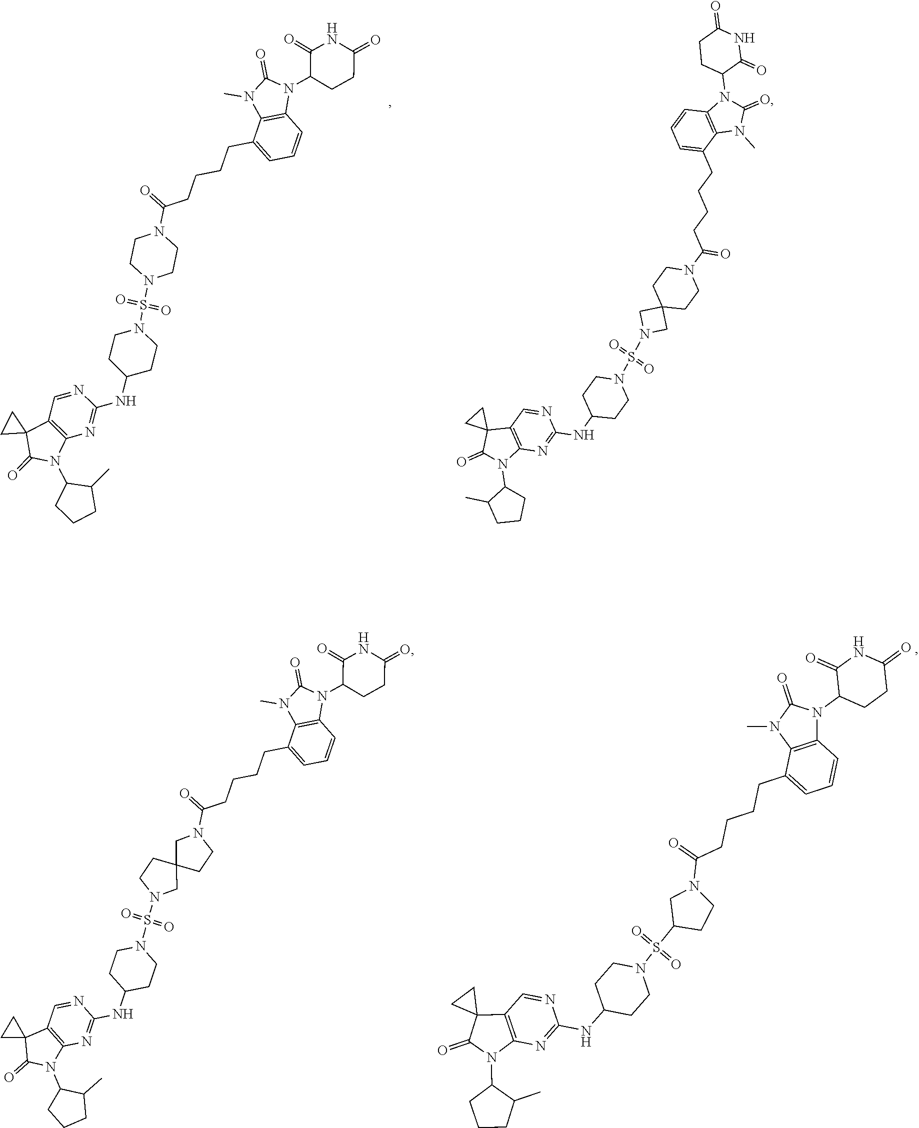 [SMILES: CC1CCCC1N1C(=O)C2(CC2)c2cnc(NC3CCN(S(=O)(=O)N4CCN(C(=O)CCCCc5cccc6c5n(C)c(=O)n6C5CCC(=O)NC5=O)CC4)CC3)nc21 ; CC1CCCC1N1C(=O)C2(CC2)c2cnc(NC3CCN(S(=O)(=O)N4CC5(CCN(C(=O)CCCCc6cccc7c6n(C)c(=O)n7C6CCC(=O)NC6=O)CC5)C4)CC3)nc21 ; CC1CCCC1N1C(=O)C2(CC2)c2cnc(NC3CCN(S(=O)(=O)N4CCC5(CCN(C(=O)CCCCc6cccc7c6n(C)c(=O)n7C6CCC(=O)NC6=O)C5)C4)CC3)nc21 ; CC1CCCC1N1C(=O)C2(CC2)c2cnc(NC3CCN(S(=O)(=O)C4CCN(C(=O)CCCCc5cccc6c5n(C)c(=O)n6C5CCC(=O)NC5=O)C4)CC3)nc21]

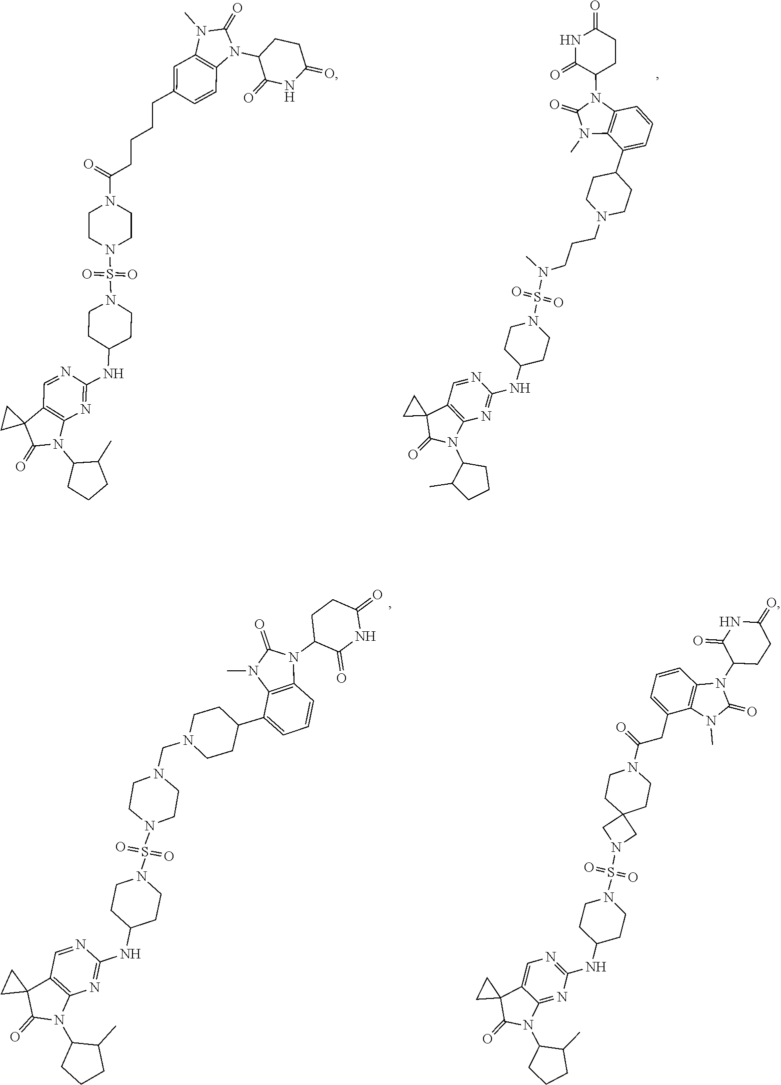 [SMILES: CC1CCCC1N1C(=O)C2(CC2)c2cnc(NC3CCN(S(=O)(=O)N4CCN(C(=O)CCCCc5ccc6c(c5)n(C)c(=O)n6C5CCC(=O)NC5=O)CC4)CC3)nc21 ; CC1CCCC1N1C(=O)C2(CC2)c2cnc(NC3CCN(S(=O)(=O)N(C)CCCN4CCC(c5cccc6c5n(C)c(=O)n6C5CCC(=O)NC5=O)CC4)CC3)nc21 ; CC1CCCC1N1C(=O)C2(CC2)c2cnc(NC3CCN(S(=O)(=O)N4CCN(CN5CCC(c6cccc7c6n(C)c(=O)n7C6CCC(=O)NC6=O)CC5)CC4)CC3)nc21 ; CC1CCCC1N1C(=O)C2(CC2)c2cnc(NC3CCN(S(=O)(=O)N4CC5(CCN(C(=O)Cc6cccc7c6n(C)c(=O)n7C6CCC(=O)NC6=O)CC5)C4)CC3)nc21]

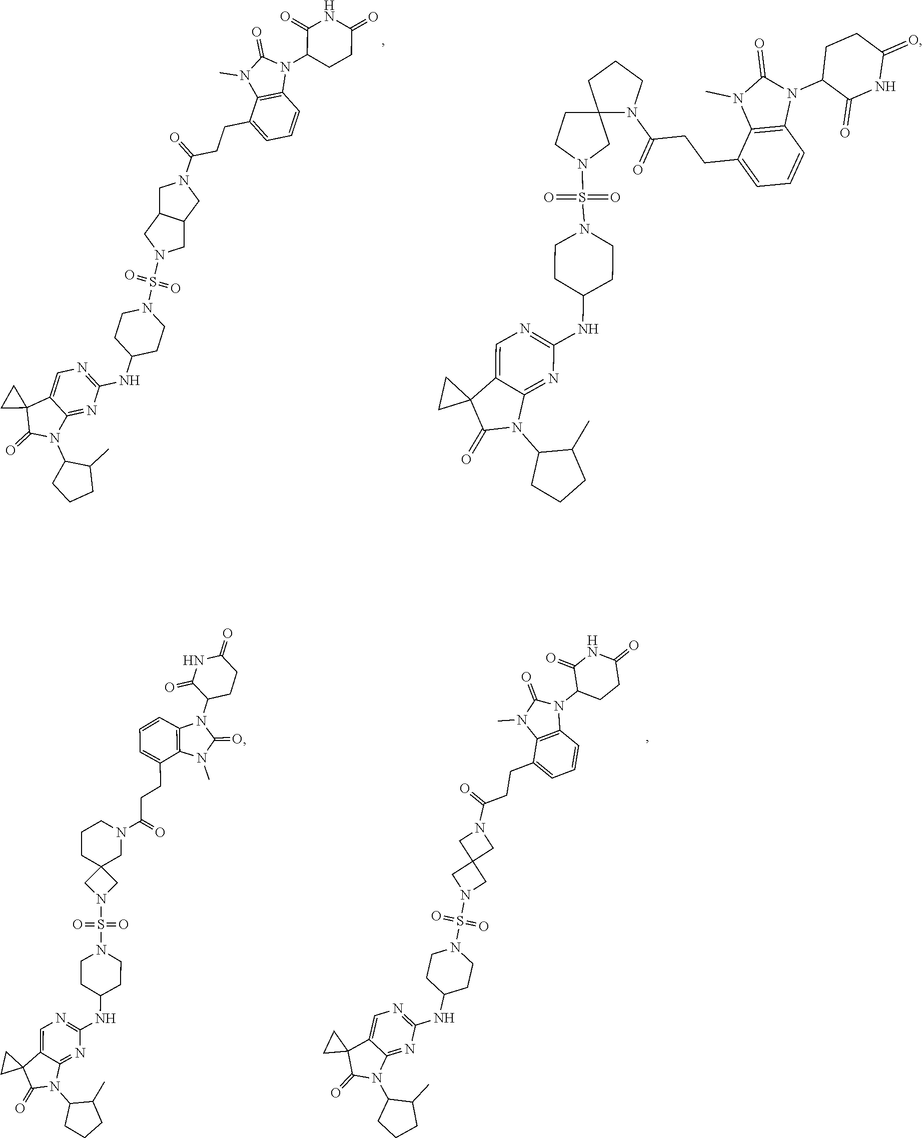 [SMILES: CC1CCCC1N1C(=O)C2(CC2)c2cnc(NC3CCN(S(=O)(=O)N4CC5CN(C(=O)CCc6cccc7c6n(C)c(=O)n7C6CCC(=O)NC6=O)CC5C4)CC3)nc21 ; CC1CCCC1N1C(=O)C2(CC2)c2cnc(NC3CCN(S(=O)(=O)N4CCC5(CCCN5C(=O)CCc5cccc6c5n(C)c(=O)n6C5CCC(=O)NC5=O)C4)CC3)nc21 ; CC1CCCC1N1C(=O)C2(CC2)c2cnc(NC3CCN(S(=O)(=O)N4CC5(CCCN(C(=O)CCc6cccc7c6n(C)c(=O)n7C6CCC(=O)NC6=O)C5)C4)CC3)nc21 ; CC1CCCC1N1C(=O)C2(CC2)c2cnc(NC3CCN(S(=O)(=O)N4CC5(CN(C(=O)CCc6cccc7c6n(C)c(=O)n7C6CCC(=O)NC6=O)C5)C4)CC3)nc21]

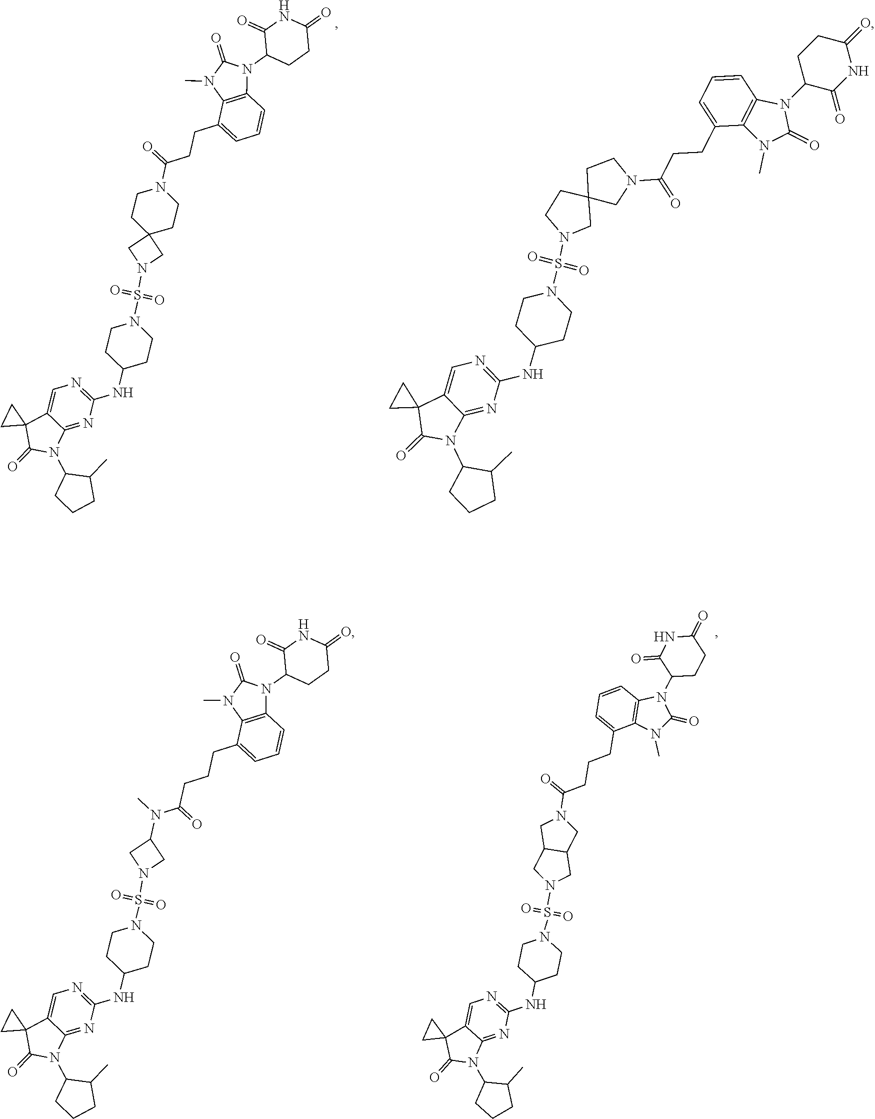 [SMILES: CC1CCCC1N1C(=O)C2(CC2)c2cnc(NC3CCN(S(=O)(=O)N4CC5(CCN(C(=O)CCc6cccc7c6n(C)c(=O)n7C6CCC(=O)NC6=O)CC5)C4)CC3)nc21 ; CC1CCCC1N1C(=O)C2(CC2)c2cnc(NC3CCN(S(=O)(=O)N4CCC5(CCN(C(=O)CCc6cccc7c6n(C)c(=O)n7C6CCC(=O)NC6=O)C5)C4)CC3)nc21 ; CC1CCCC1N1C(=O)C2(CC2)c2cnc(NC3CCN(S(=O)(=O)N4CC(N(C)C(=O)CCCc5cccc6c5n(C)c(=O)n6C5CCC(=O)NC5=O)C4)CC3)nc21 ; CC1CCCC1N1C(=O)C2(CC2)c2cnc(NC3CCN(S(=O)(=O)N4CC5CN(C(=O)CCCc6cccc7c6n(C)c(=O)n7C6CCC(=O)NC6=O)CC5C4)CC3)nc21]

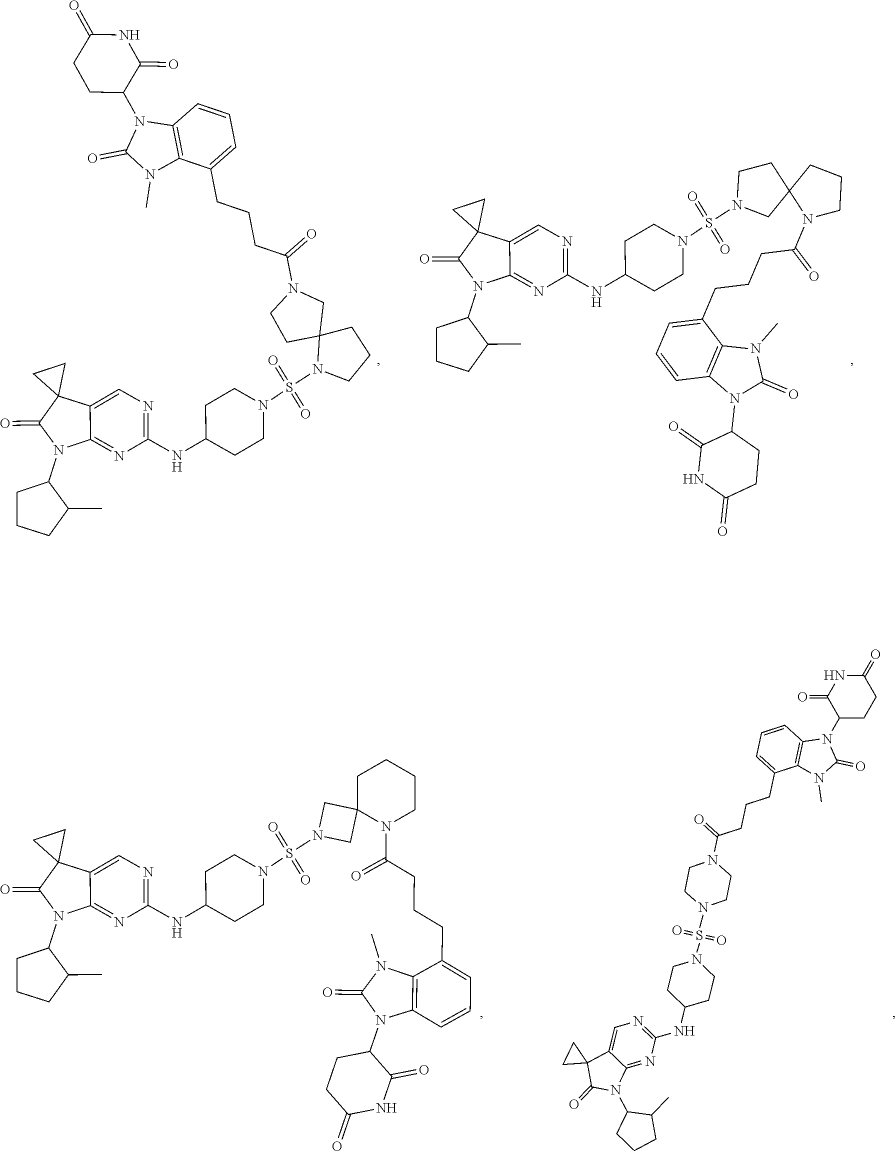 [SMILES: CC1CCCC1N1C(=O)C2(CC2)c2cnc(NC3CCN(S(=O)(=O)N4CCCC45CCN(C(=O)CCCc4cccc6c4n(C)c(=O)n6C4CCC(=O)NC4=O)C5)CC3)nc21 ; CC1CCCC1N1C(=O)C2(CC2)c2cnc(NC3CCN(S(=O)(=O)N4CCC5(CCCN5C(=O)CCCc5cccc6c5n(C)c(=O)n6C5CCC(=O)NC5=O)C4)CC3)nc21 ; CC1CCCC1N1C(=O)C2(CC2)c2cnc(NC3CCN(S(=O)(=O)N4CC5(CCCCN5C(=O)CCCc5cccc6c5n(C)c(=O)n6C5CCC(=O)NC5=O)C4)CC3)nc21 ; CC1CCCC1N1C(=O)C2(CC2)c2cnc(NC3CCN(S(=O)(=O)N4CCN(C(=O)CCCc5cccc6c5n(C)c(=O)n6C5CCC(=O)NC5=O)CC4)CC3)nc21]

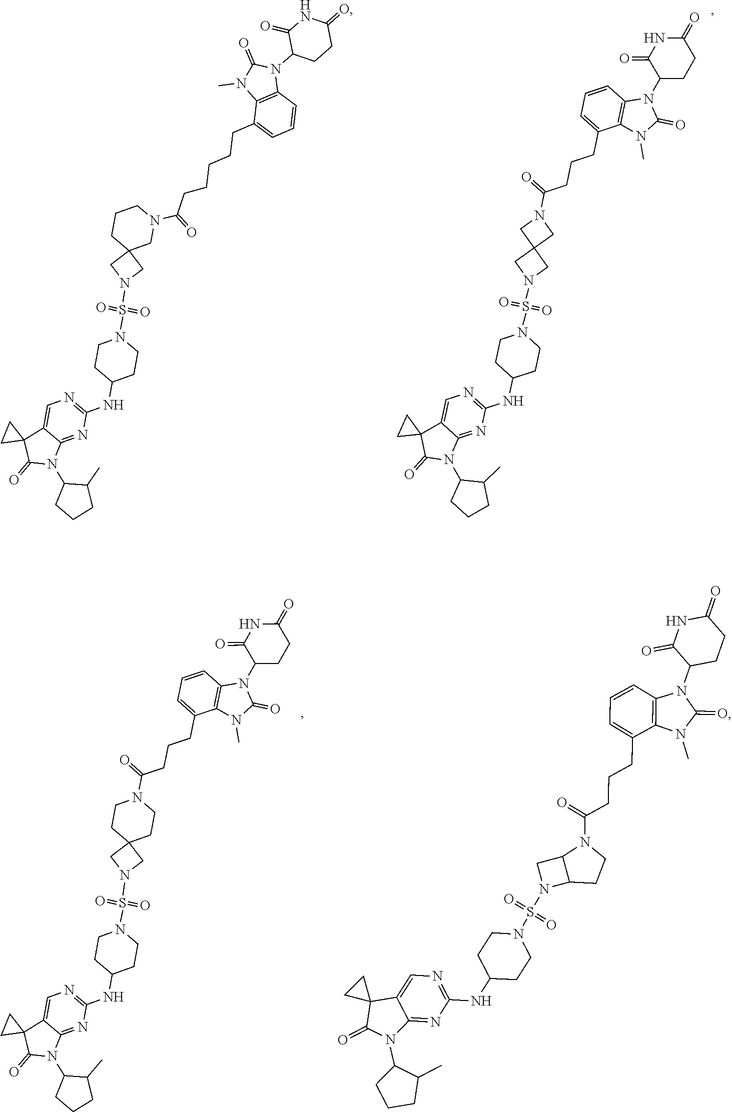 [SMILES: CC1CCCC1N1C(=O)C2(CC2)c2cnc(NC3CCN(S(=O)(=O)N4CC5(CCCN(C(=O)CCCCCc6cccc7c6n(C)c(=O)n7C6CCC(=O)NC6=O)C5)C4)CC3)nc21 ; CC1CCCC1N1C(=O)C2(CC2)c2cnc(NC3CCN(S(=O)(=O)N4CC5(CN(C(=O)CCCc6cccc7c6n(C)c(=O)n7C6CCC(=O)NC6=O)C5)C4)CC3)nc21 ; CC1CCCC1N1C(=O)C2(CC2)c2cnc(NC3CCN(S(=O)(=O)N4CC5(CCN(C(=O)CCCc6cccc7c6n(C)c(=O)n7C6CCC(=O)NC6=O)CC5)C4)CC3)nc21 ; CC1CCCC1N1C(=O)C2(CC2)c2cnc(NC3CCN(S(=O)(=O)N4CC5C4CCN5C(=O)CCCc4cccc5c4n(C)c(=O)n5C4CCC(=O)NC4=O)CC3)nc21]

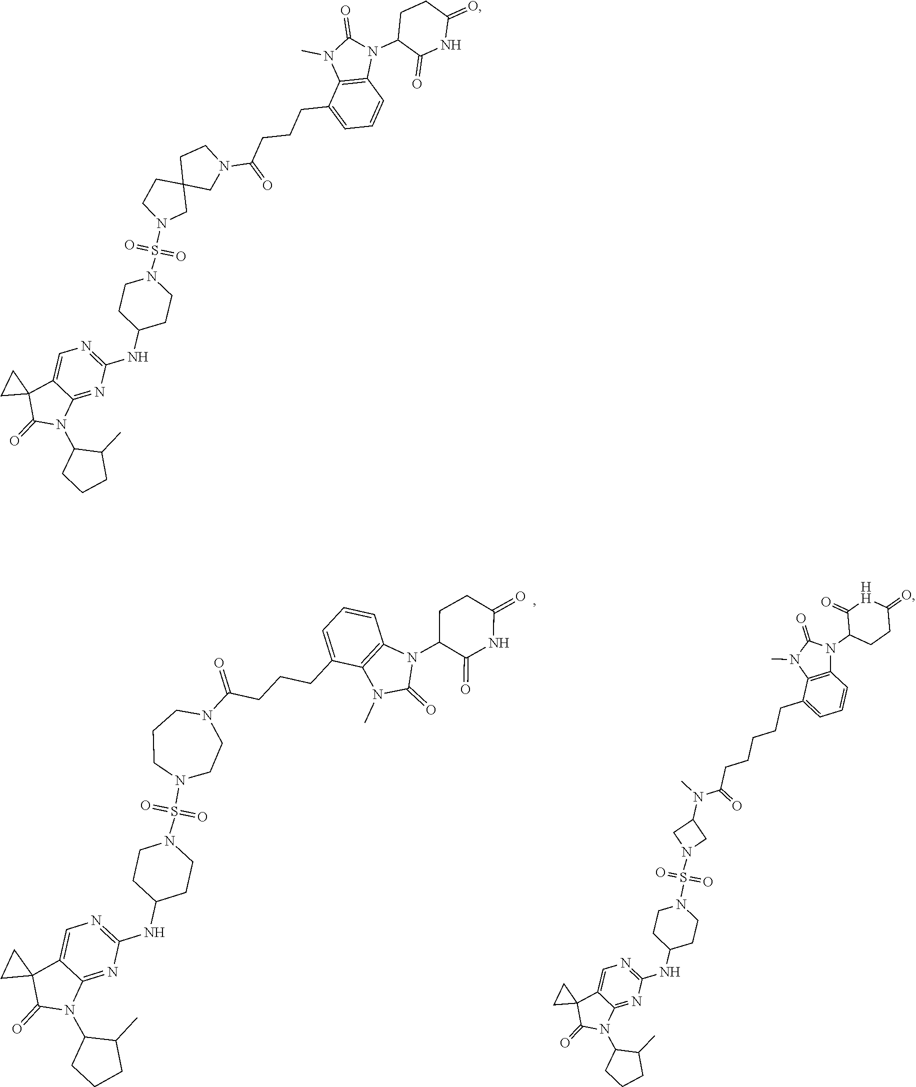

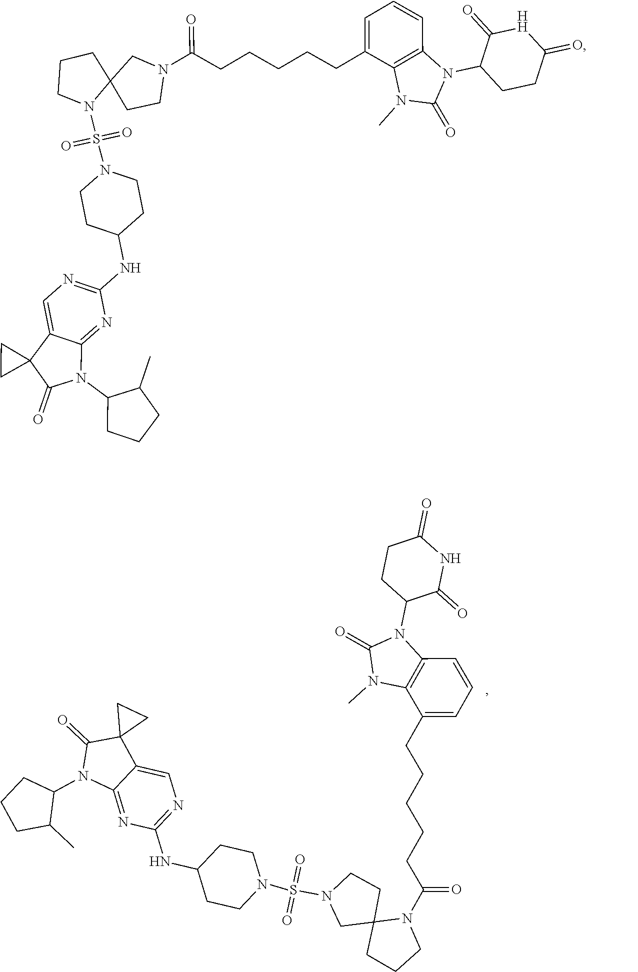

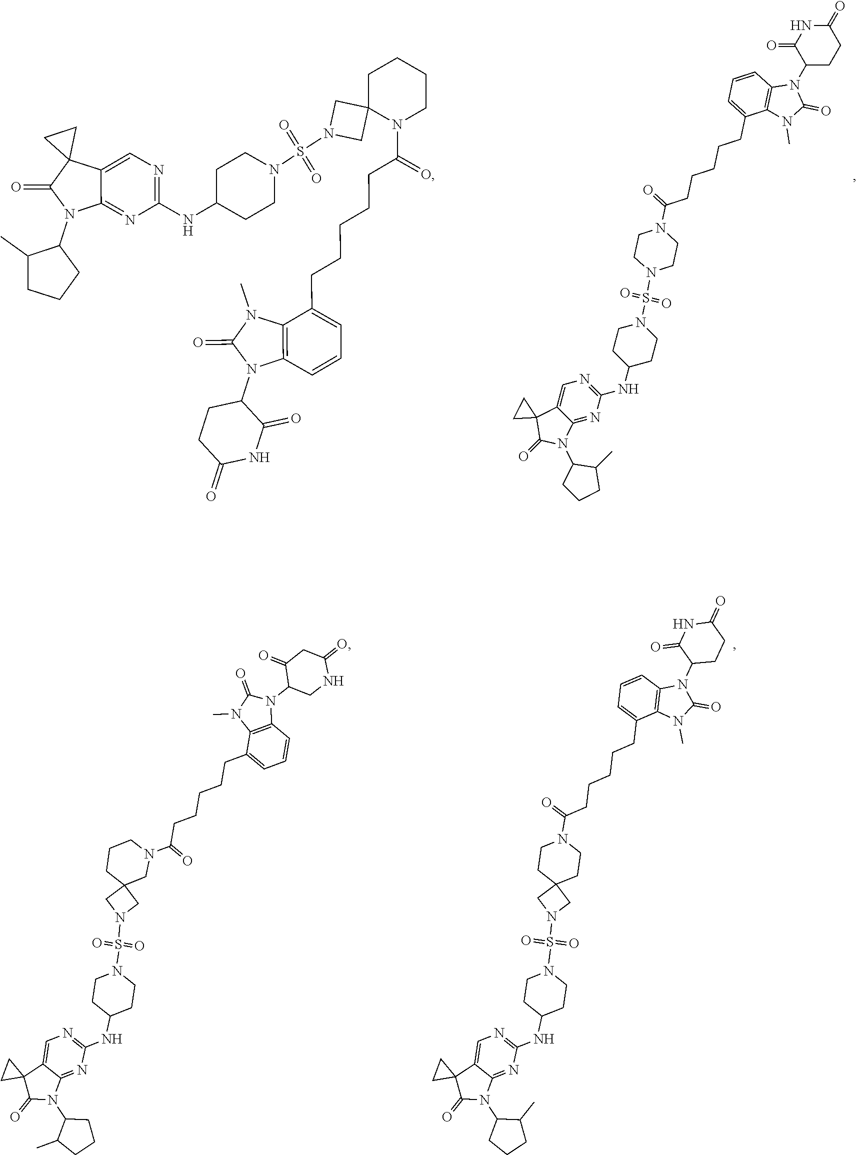 [SMILES: CC1CCCC1N1C(=O)C2(CC2)c2cnc(NC3CCN(S(=O)(=O)N4CC5(CCCCN5C(=O)CCCCCc5cccc6c5n(C)c(=O)n6C5CCC(=O)NC5=O)C4)CC3)nc21 ; CC1CCCC1N1C(=O)C2(CC2)c2cnc(NC3CCN(S(=O)(=O)N4CCN(C(=O)CCCCCc5cccc6c5n(C)c(=O)n6C5CCC(=O)NC5=O)CC4)CC3)nc21 ; CC1CCCC1N1C(=O)C2(CC2)c2cnc(NC3CCN(S(=O)(=O)N4CC5(CCCN(C(=O)CCCCCc6cccc7c6n(C)c(=O)n7C6CNC(=O)CC6=O)C5)C4)CC3)nc21 ; CC1CCCC1N1C(=O)C2(CC2)c2cnc(NC3CCN(S(=O)(=O)N4CC5(CCN(C(=O)CCCCCc6cccc7c6n(C)c(=O)n7C6CCC(=O)NC6=O)CC5)C4)CC3)nc21]

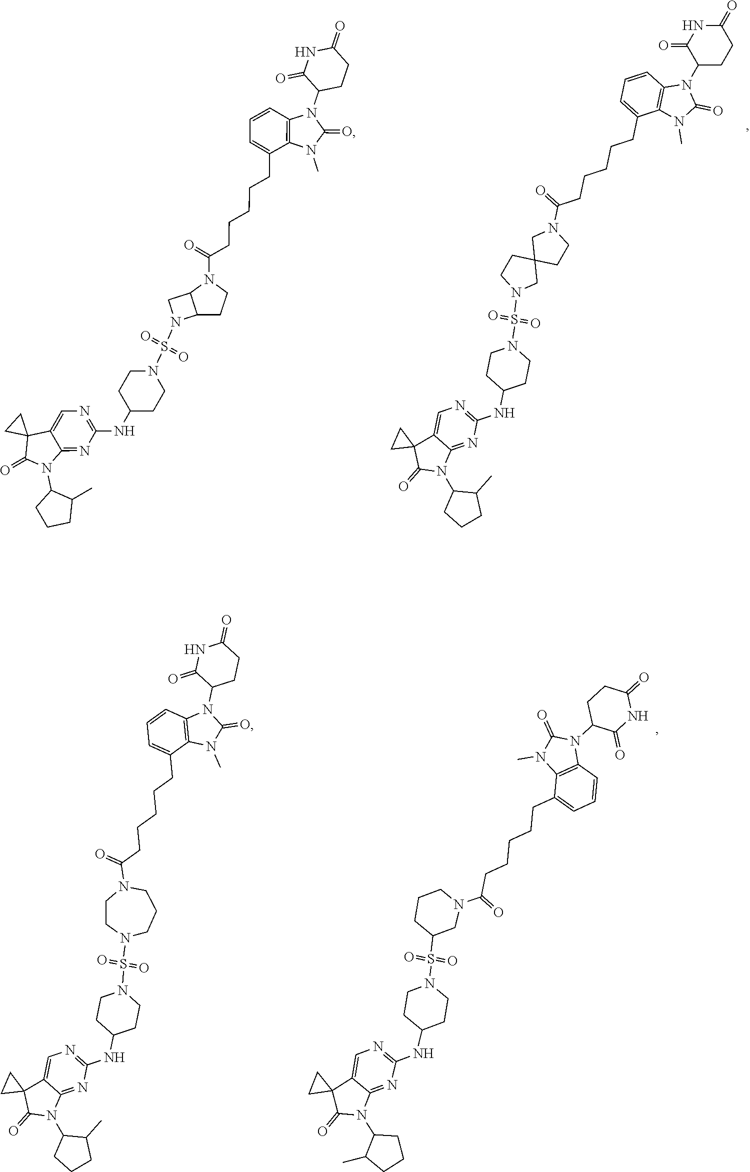 [SMILES: CC1CCCC1N1C(=O)C2(CC2)c2cnc(NC3CCN(S(=O)(=O)N4CC5C4CCN5C(=O)CCCCCc4cccc5c4n(C)c(=O)n5C4CCC(=O)NC4=O)CC3)nc21 ; CC1CCCC1N1C(=O)C2(CC2)c2cnc(NC3CCN(S(=O)(=O)N4CCC5(CCN(C(=O)CCCCCc6cccc7c6n(C)c(=O)n7C6CCC(=O)NC6=O)C5)C4)CC3)nc21 ; CC1CCCC1N1C(=O)C2(CC2)c2cnc(NC3CCN(S(=O)(=O)N4CCCN(C(=O)CCCCCc5cccc6c5n(C)c(=O)n6C5CCC(=O)NC5=O)CC4)CC3)nc21 ; CC1CCCC1N1C(=O)C2(CC2)c2cnc(NC3CCN(S(=O)(=O)C4CCCN(C(=O)CCCCCc5cccc6c5n(C)c(=O)n6C5CCC(=O)NC5=O)C4)CC3)nc21]

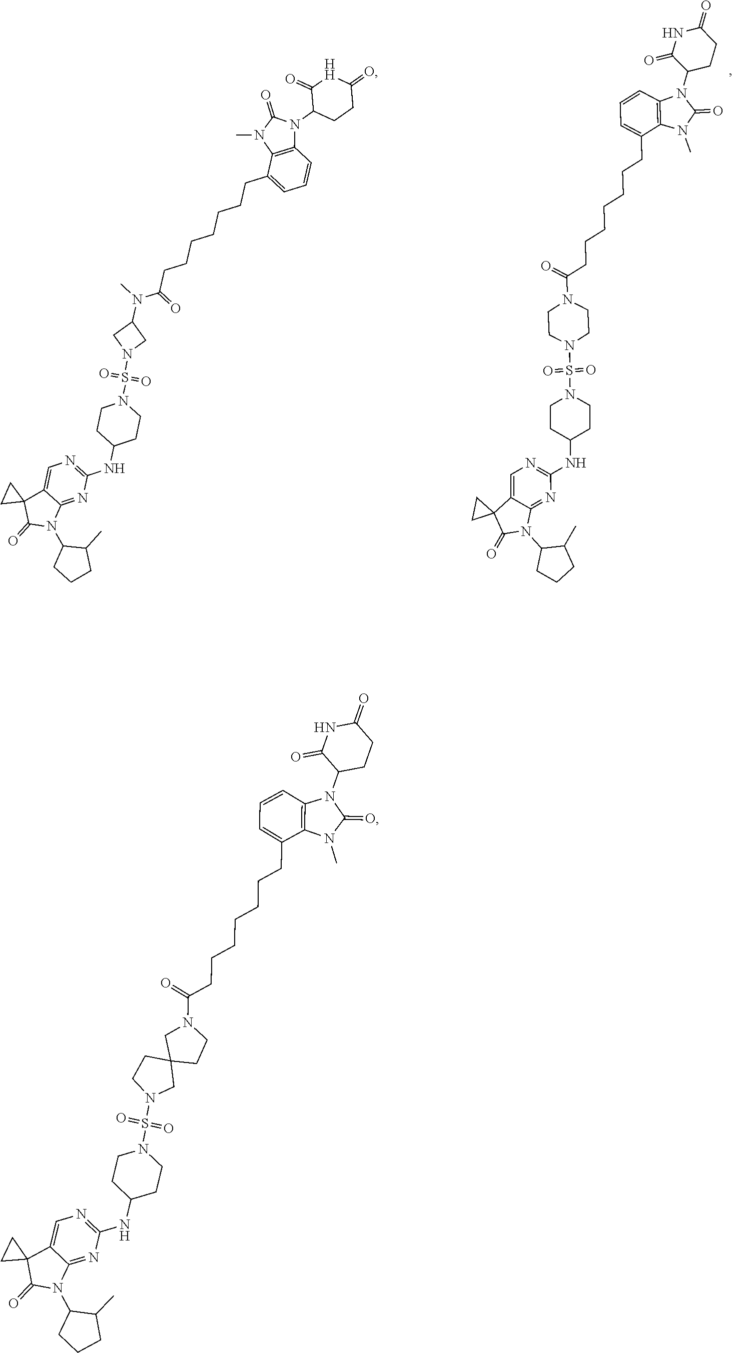

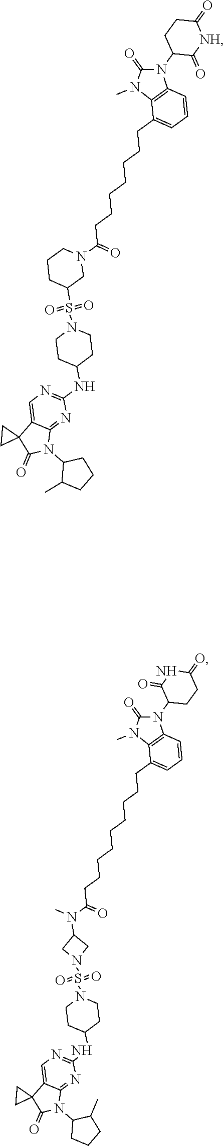 [SMILES: CC1CCCC1N1C(=O)C2(CC2)c2cnc(NC3CCN(S(=O)(=O)C4CCCN(C(=O)CCCCCCCc5cccc6c5n(C)c(=O)n6C5CCC(=O)NC5=O)C4)CC3)nc21 ; CC1CCCC1N1C(=O)C2(CC2)c2cnc(NC3CCN(S(=O)(=O)N4CC(N(C)C(=O)CCCCCCCCCc5cccc6c5n(C)c(=O)n6C5CCC(=O)NC5=O)C4)CC3)nc21]

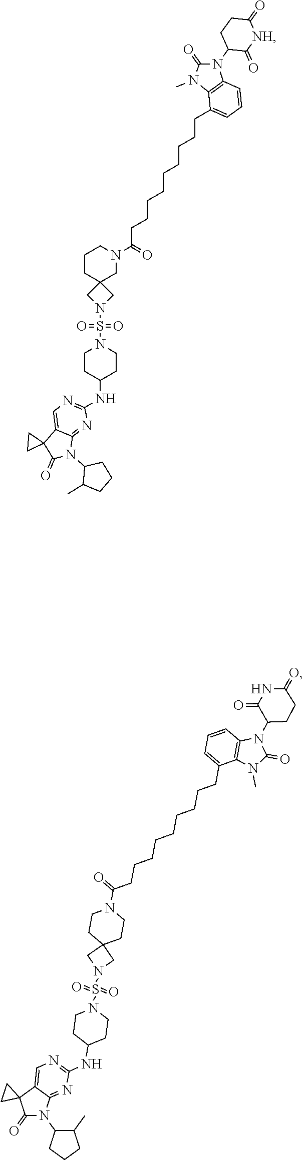 [SMILES: CC1CCCC1N1C(=O)C2(CC2)c2cnc(NC3CCN(S(=O)(=O)N4CC5(CCCN(C(=O)CCCCCCCCCc6cccc7c6n(C)c(=O)n7C6CCC(=O)NC6=O)C5)C4)CC3)nc21 ; CC1CCCC1N1C(=O)C2(CC2)c2cnc(NC3CCN(S(=O)(=O)N4CC5(CCN(C(=O)CCCCCCCCCc6cccc7c6n(C)c(=O)n7C6CCC(=O)NC6=O)CC5)C4)CC3)nc21]

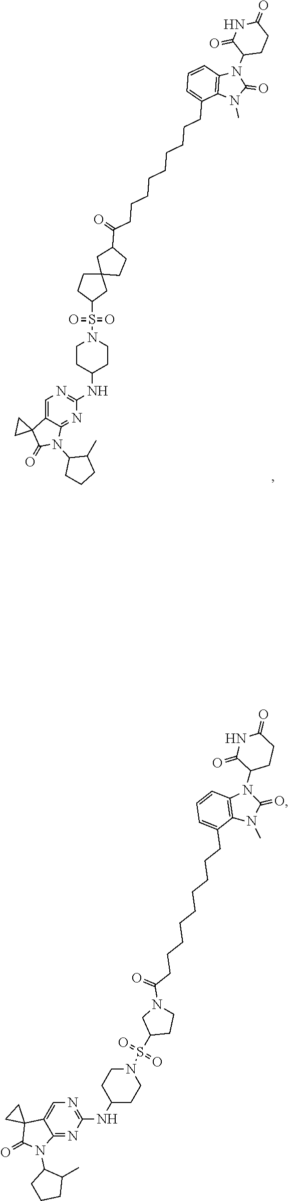 [SMILES: CC1CCCC1N1C(=O)C2(CC2)c2cnc(NC3CCN(S(=O)(=O)C4CCC5(CCC(C(=O)CCCCCCCCCc6cccc7c6n(C)c(=O)n7C6CCC(=O)NC6=O)C5)C4)CC3)nc21 ; CC1CCCC1N1C(=O)C2(CC2)c2cnc(NC3CCN(S(=O)(=O)C4CCN(C(=O)CCCCCCCCCc5cccc6c5n(C)c(=O)n6C5CCC(=O)NC5=O)C4)CC3)nc21]

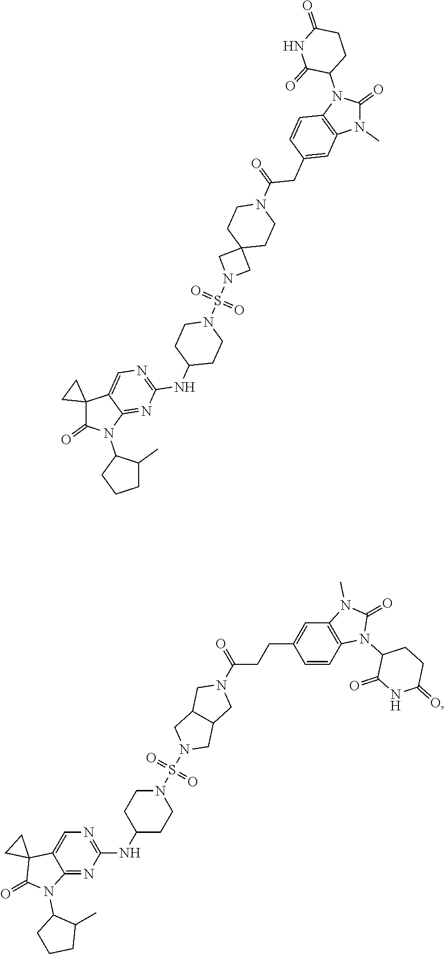 [SMILES: CC1CCCC1N1C(=O)C2(CC2)c2cnc(NC3CCN(S(=O)(=O)N4CC5(CCN(C(=O)Cc6ccc7c(c6)n(C)c(=O)n7C6CCC(=O)NC6=O)CC5)C4)CC3)nc21 ; CC1CCCC1N1C(=O)C2(CC2)c2cnc(NC3CCN(S(=O)(=O)N4CC5CN(C(=O)CCc6ccc7c(c6)n(C)c(=O)n7C6CCC(=O)NC6=O)CC5C4)CC3)nc21]

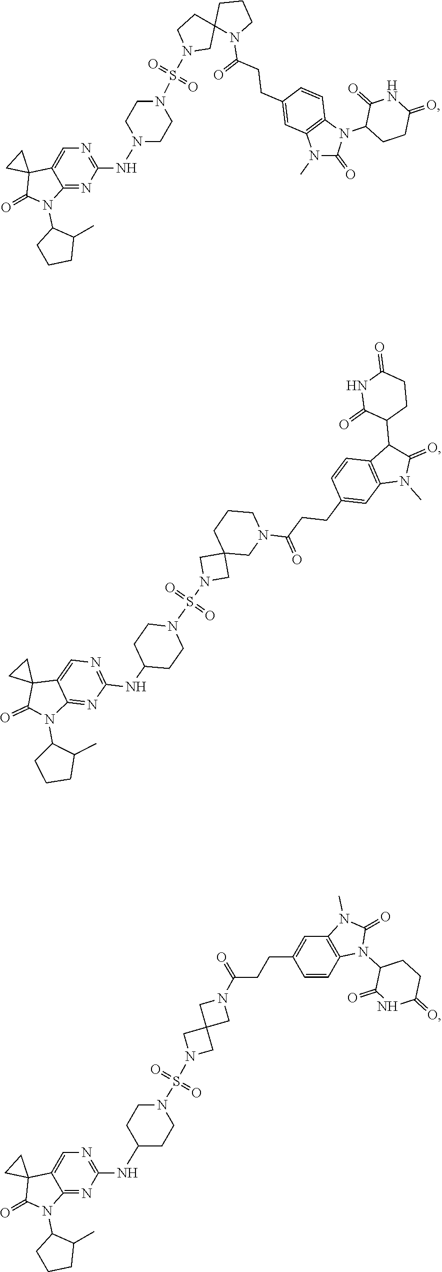 [SMILES: CC1CCCC1N1C(=O)C2(CC2)c2cnc(NN3CCN(S(=O)(=O)N4CCC5(CCCN5C(=O)CCc5ccc6c(c5)n(C)c(=O)n6C5CCC(=O)NC5=O)C4)CC3)nc21 ; CC1CCCC1N1C(=O)C2(CC2)c2cnc(NC3CCN(S(=O)(=O)N4CC5(CCCN(C(=O)CCc6ccc7c(c6)N(C)C(=O)C7C6CCC(=O)NC6=O)C5)C4)CC3)nc21 ; CC1CCCC1N1C(=O)C2(CC2)c2cnc(NC3CCN(S(=O)(=O)N4CC5(CN(C(=O)CCc6ccc7c(c6)n(C)c(=O)n7C6CCC(=O)NC6=O)C5)C4)CC3)nc21]

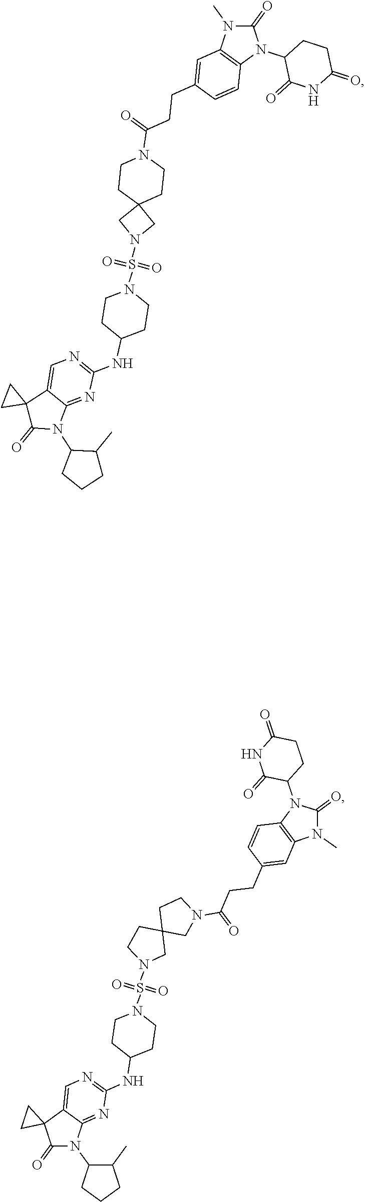 [SMILES: CC1CCCC1N1C(=O)C2(CC2)c2cnc(NC3CCN(S(=O)(=O)N4CC5(CCN(C(=O)CCc6ccc7c(c6)n(C)c(=O)n7C6CCC(=O)NC6=O)CC5)C4)CC3)nc21 ; CC1CCCC1N1C(=O)C2(CC2)c2cnc(NC3CCN(S(=O)(=O)N4CCC5(CCN(C(=O)CCc6ccc7c(c6)n(C)c(=O)n7C6CCC(=O)NC6=O)C5)C4)CC3)nc21]

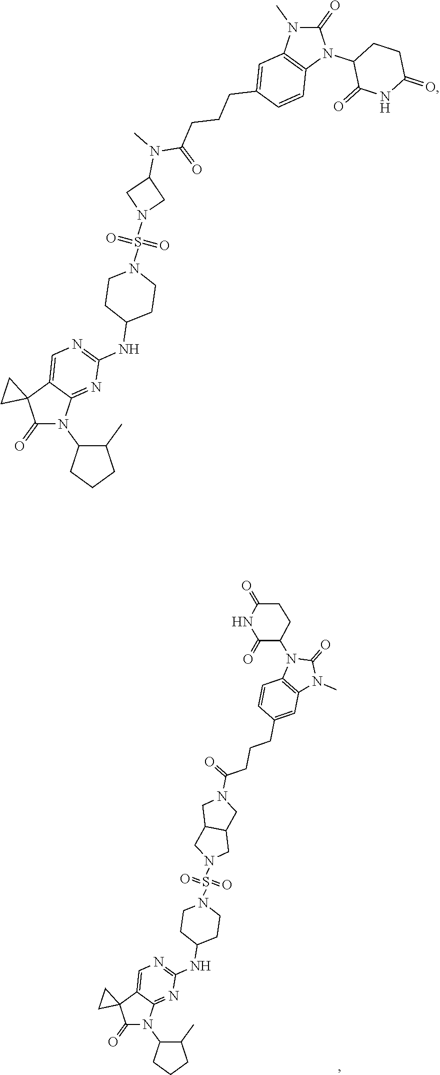 [SMILES: CC1CCCC1N1C(=O)C2(CC2)c2cnc(NC3CCN(S(=O)(=O)N4CC(N(C)C(=O)CCCc5ccc6c(c5)n(C)c(=O)n6C5CCC(=O)NC5=O)C4)CC3)nc21 ; CC1CCCC1N1C(=O)C2(CC2)c2cnc(NC3CCN(S(=O)(=O)N4CC5CN(C(=O)CCCc6ccc7c(c6)n(C)c(=O)n7C6CCC(=O)NC6=O)CC5C4)CC3)nc21]

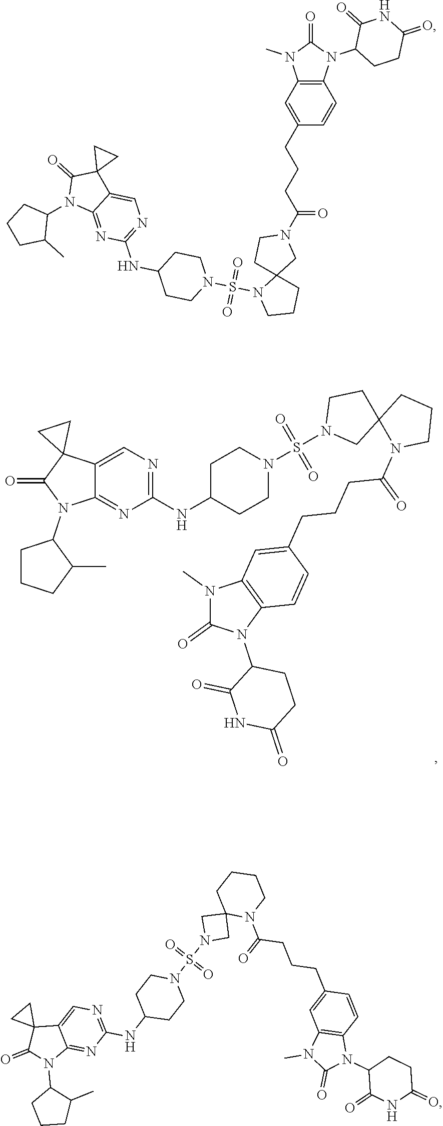 [SMILES: CC1CCCC1N1C(=O)C2(CC2)c2cnc(NC3CCN(S(=O)(=O)N4CCCC45CCN(C(=O)CCCc4ccc6c(c4)n(C)c(=O)n6C4CCC(=O)NC4=O)C5)CC3)nc21 ; CC1CCCC1N1C(=O)C2(CC2)c2cnc(NC3CCN(S(=O)(=O)N4CCC5(CCCN5C(=O)CCCc5ccc6c(c5)n(C)c(=O)n6C5CCC(=O)NC5=O)C4)CC3)nc21 ; CC1CCCC1N1C(=O)C2(CC2)c2cnc(NC3CCN(S(=O)(=O)N4CC5(CCCCN5C(=O)CCCc5ccc6c(c5)n(C)c(=O)n6C5CCC(=O)NC5=O)C4)CC3)nc21]

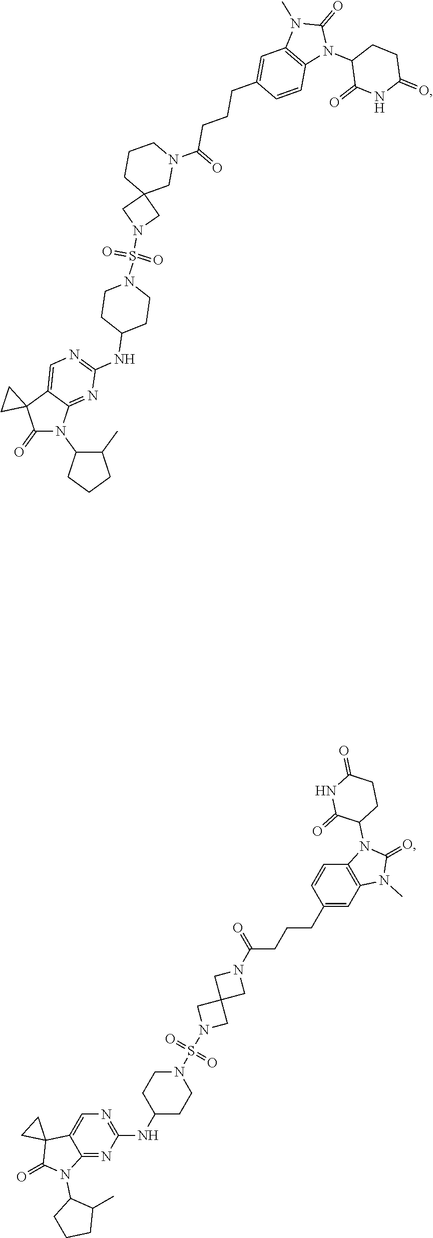 [SMILES: CC1CCCC1N1C(=O)C2(CC2)c2cnc(NC3CCN(S(=O)(=O)N4CC5(CCCN(C(=O)CCCc6ccc7c(c6)n(C)c(=O)n7C6CCC(=O)NC6=O)C5)C4)CC3)nc21 ; CC1CCCC1N1C(=O)C2(CC2)c2cnc(NC3CCN(S(=O)(=O)N4CC5(CN(C(=O)CCCc6ccc7c(c6)n(C)c(=O)n7C6CCC(=O)NC6=O)C5)C4)CC3)nc21]

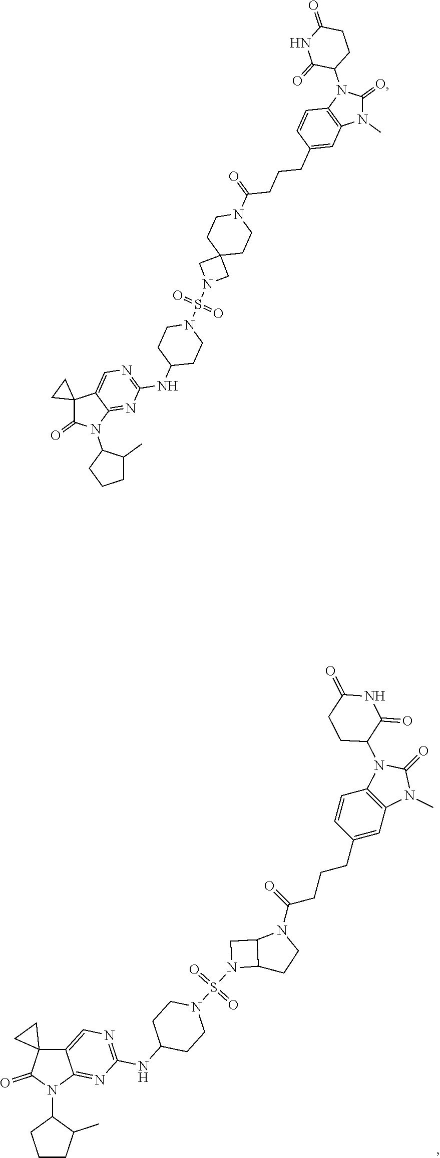 [SMILES: CC1CCCC1N1C(=O)C2(CC2)c2cnc(NC3CCN(S(=O)(=O)N4CC5(CCN(C(=O)CCCc6ccc7c(c6)n(C)c(=O)n7C6CCC(=O)NC6=O)CC5)C4)CC3)nc21 ; CC1CCCC1N1C(=O)C2(CC2)c2cnc(NC3CCN(S(=O)(=O)N4CC5C4CCN5C(=O)CCCc4ccc5c(c4)n(C)c(=O)n5C4CCC(=O)NC4=O)CC3)nc21]

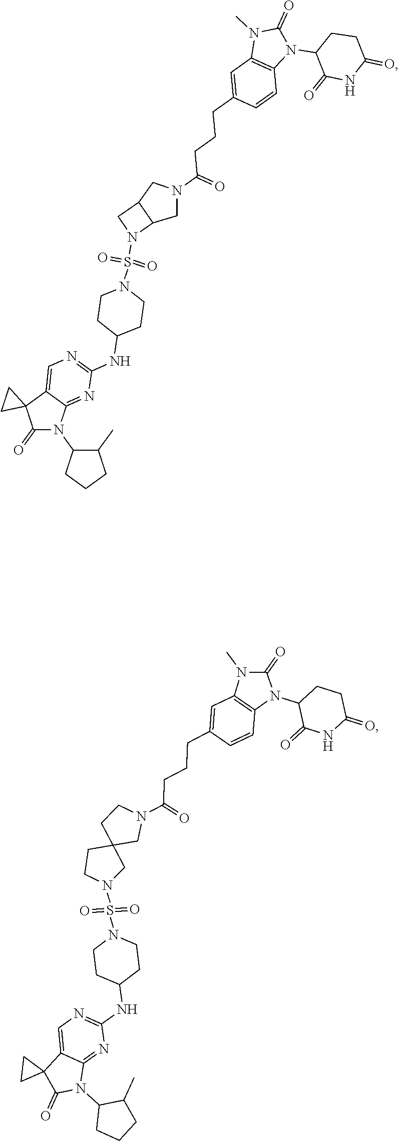 [SMILES: CC1CCCC1N1C(=O)C2(CC2)c2cnc(NC3CCN(S(=O)(=O)N4CC5CN(C(=O)CCCc6ccc7c(c6)n(C)c(=O)n7C6CCC(=O)NC6=O)CC54)CC3)nc21 ; CC1CCCC1N1C(=O)C2(CC2)c2cnc(NC3CCN(S(=O)(=O)N4CCC5(CCN(C(=O)CCCc6ccc7c(c6)n(C)c(=O)n7C6CCC(=O)NC6=O)C5)C4)CC3)nc21]

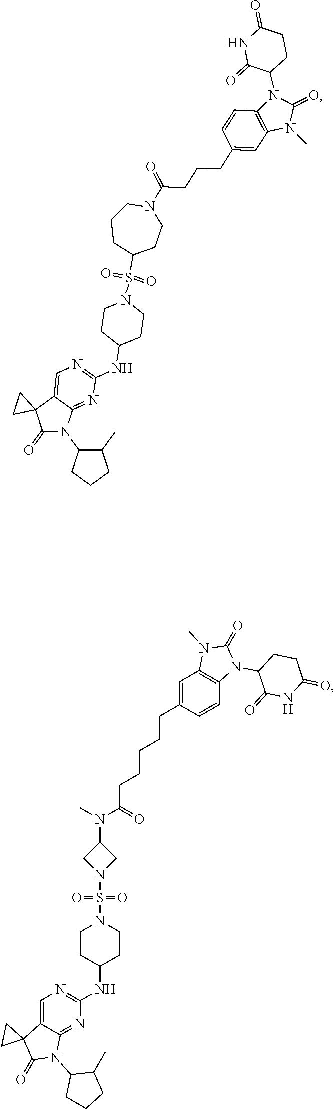 [SMILES: CC1CCCC1N1C(=O)C2(CC2)c2cnc(NC3CCN(S(=O)(=O)C4CCCN(C(=O)CCCc5ccc6c(c5)n(C)c(=O)n6C5CCC(=O)NC5=O)CC4)CC3)nc21 ; CC1CCCC1N1C(=O)C2(CC2)c2cnc(NC3CCN(S(=O)(=O)N4CC(N(C)C(=O)CCCCCc5ccc6c(c5)n(C)c(=O)n6C5CCC(=O)NC5=O)C4)CC3)nc21]

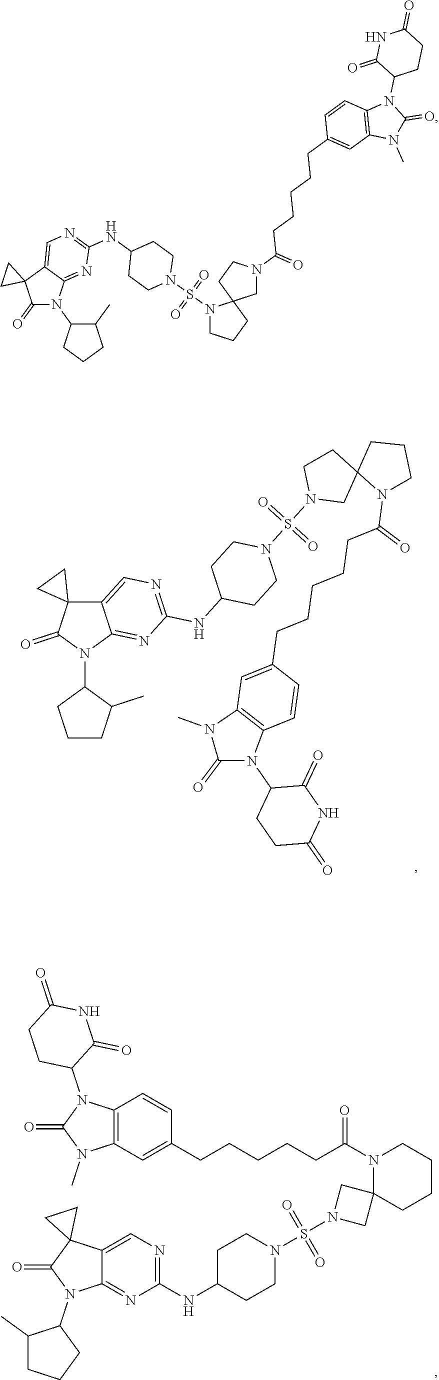 [SMILES: CC1CCCC1N1C(=O)C2(CC2)c2cnc(NC3CCN(S(=O)(=O)N4CCCC45CCN(C(=O)CCCCCc4ccc6c(c4)n(C)c(=O)n6C4CCC(=O)NC4=O)C5)CC3)nc21 ; CC1CCCC1N1C(=O)C2(CC2)c2cnc(NC3CCN(S(=O)(=O)N4CCC5(CCCN5C(=O)CCCCCc5ccc6c(c5)n(C)c(=O)n6C5CCC(=O)NC5=O)C4)CC3)nc21 ; CC1CCCC1N1C(=O)C2(CC2)c2cnc(NC3CCN(S(=O)(=O)N4CC5(CCCCN5C(=O)CCCCCc5ccc6c(c5)n(C)c(=O)n6C5CCC(=O)NC5=O)C4)CC3)nc21]

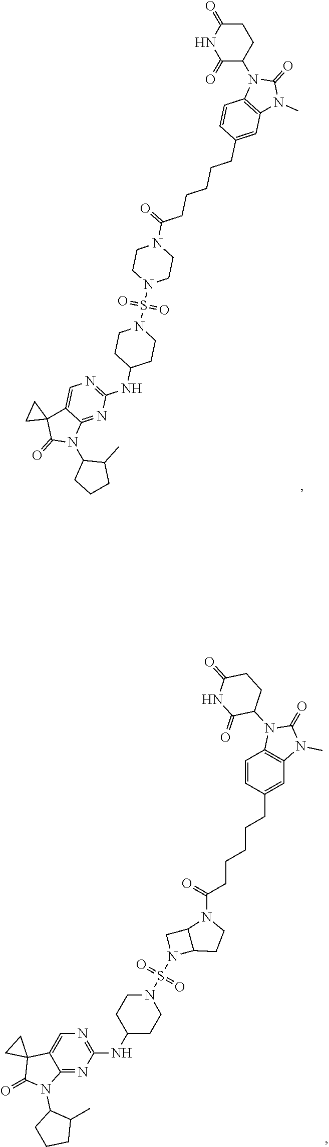 [SMILES: CC1CCCC1N1C(=O)C2(CC2)c2cnc(NC3CCN(S(=O)(=O)N4CCN(C(=O)CCCCCc5ccc6c(c5)n(C)c(=O)n6C5CCC(=O)NC5=O)CC4)CC3)nc21 ; CC1CCCC1N1C(=O)C2(CC2)c2cnc(NC3CCN(S(=O)(=O)N4CC5C4CCN5C(=O)CCCCCc4ccc5c(c4)n(C)c(=O)n5C4CCC(=O)NC4=O)CC3)nc21]

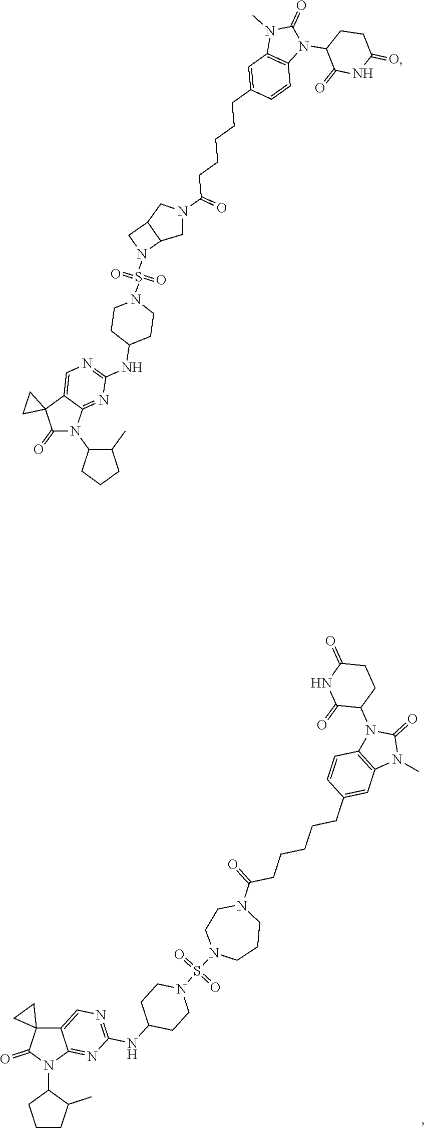 [SMILES: CC1CCCC1N1C(=O)C2(CC2)c2cnc(NC3CCN(S(=O)(=O)N4CC5CN(C(=O)CCCCCc6ccc7c(c6)n(C)c(=O)n7C6CCC(=O)NC6=O)CC54)CC3)nc21 ; CC1CCCC1N1C(=O)C2(CC2)c2cnc(NC3CCN(S(=O)(=O)N4CCCN(C(=O)CCCCCc5ccc6c(c5)n(C)c(=O)n6C5CCC(=O)NC5=O)CC4)CC3)nc21]

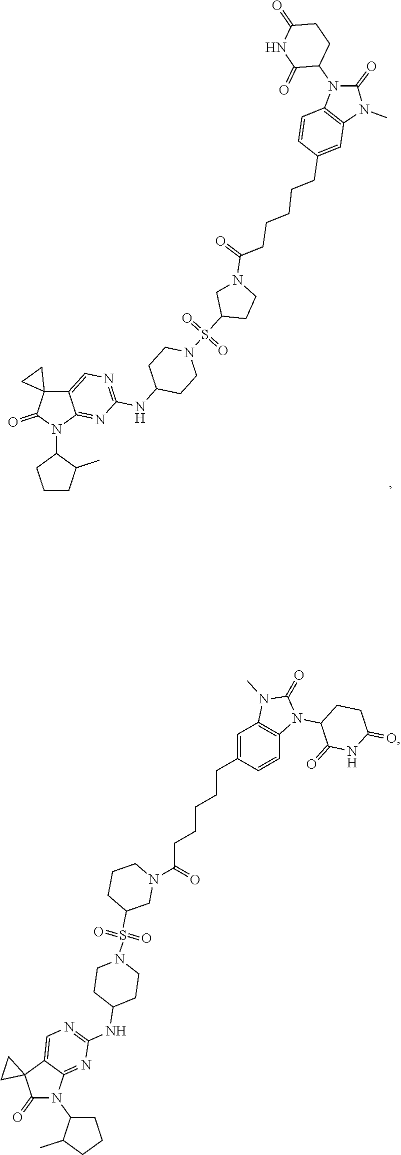 [SMILES: CC1CCCC1N1C(=O)C2(CC2)c2cnc(NC3CCN(S(=O)(=O)C4CCN(C(=O)CCCCCc5ccc6c(c5)n(C)c(=O)n6C5CCC(=O)NC5=O)C4)CC3)nc21 ; CC1CCCC1N1C(=O)C2(CC2)c2cnc(NC3CCN(S(=O)(=O)C4CCCN(C(=O)CCCCCc5ccc6c(c5)n(C)c(=O)n6C5CCC(=O)NC5=O)C4)CC3)nc21]

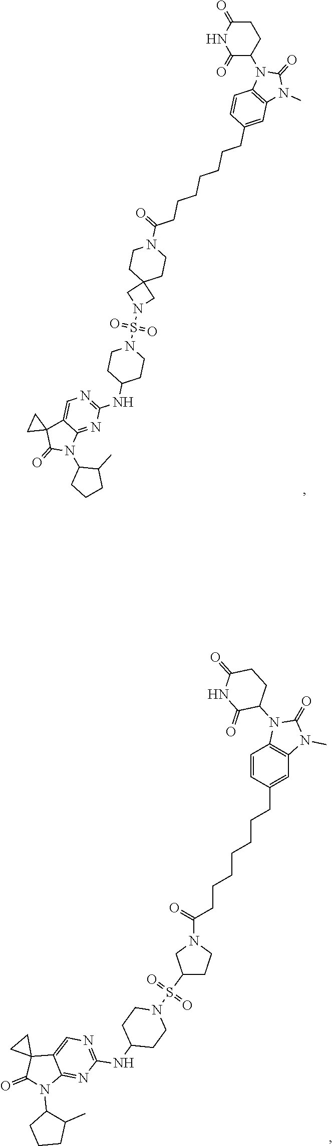 [SMILES: CC1CCCC1N1C(=O)C2(CC2)c2cnc(NC3CCN(S(=O)(=O)N4CC5(CCN(C(=O)CCCCCCCc6ccc7c(c6)n(C)c(=O)n7C6CCC(=O)NC6=O)CC5)C4)CC3)nc21 ; CC1CCCC1N1C(=O)C2(CC2)c2cnc(NC3CCN(S(=O)(=O)C4CCN(C(=O)CCCCCCCc5ccc6c(c5)n(C)c(=O)n6C5CCC(=O)NC5=O)C4)CC3)nc21]

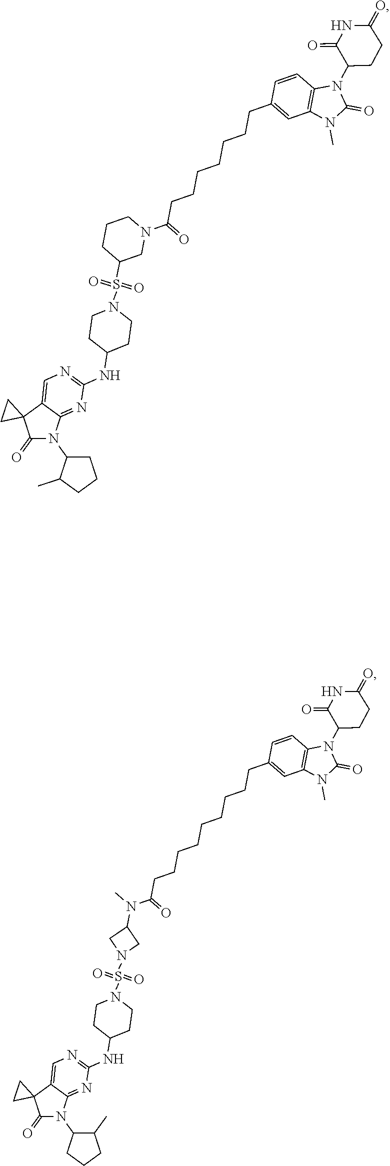 [SMILES: CC1CCCC1N1C(=O)C2(CC2)c2cnc(NC3CCN(S(=O)(=O)C4CCCN(C(=O)CCCCCCCc5ccc6c(c5)n(C)c(=O)n6C5CCC(=O)NC5=O)C4)CC3)nc21 ; CC1CCCC1N1C(=O)C2(CC2)c2cnc(NC3CCN(S(=O)(=O)N4CC(N(C)C(=O)CCCCCCCCCc5ccc6c(c5)n(C)c(=O)n6C5CCC(=O)NC5=O)C4)CC3)nc21]

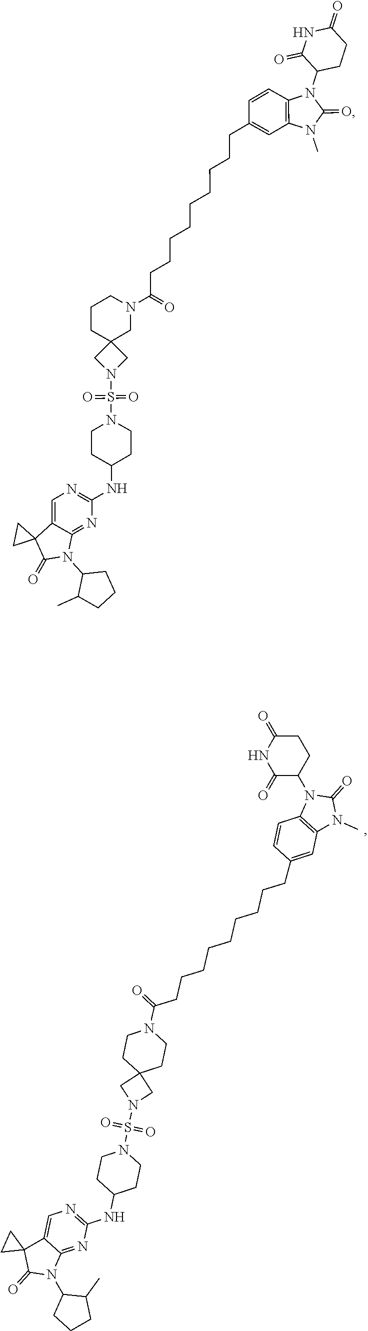 [SMILES: CC1CCCC1N1C(=O)C2(CC2)c2cnc(NC3CCN(S(=O)(=O)N4CC5(CCCN(C(=O)CCCCCCCCCc6ccc7c(c6)n(C)c(=O)n7C6CCC(=O)NC6=O)C5)C4)CC3)nc21 ; CC1CCCC1N1C(=O)C2(CC2)c2cnc(NC3CCN(S(=O)(=O)N4CC5(CCN(C(=O)CCCCCCCCCc6ccc7c(c6)n(C)c(=O)n7C6CCC(=O)NC6=O)CC5)C4)CC3)nc21]

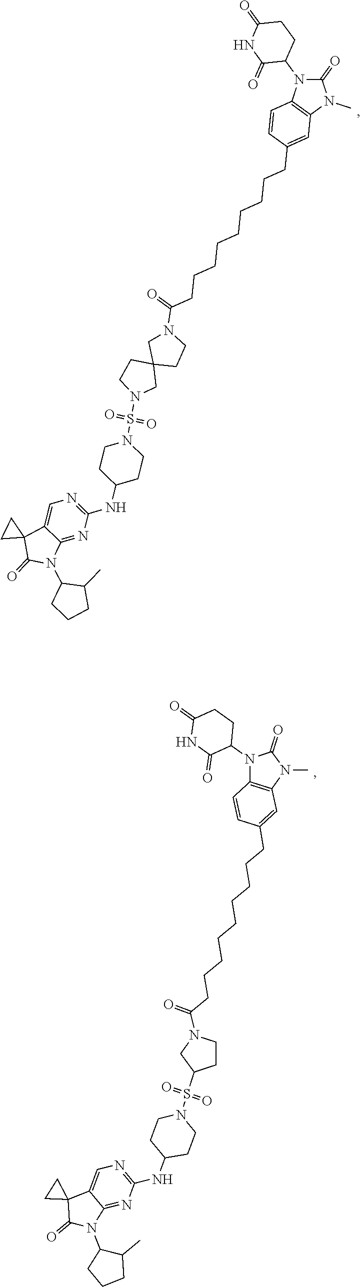 [SMILES: CC1CCCC1N1C(=O)C2(CC2)c2cnc(NC3CCN(S(=O)(=O)N4CCC5(CCN(C(=O)CCCCCCCCCc6ccc7c(c6)n(C)c(=O)n7C6CCC(=O)NC6=O)C5)C4)CC3)nc21 ; CC1CCCC1N1C(=O)C2(CC2)c2cnc(NC3CCN(S(=O)(=O)C4CCN(C(=O)CCCCCCCCCc5ccc6c(c5)n(C)c(=O)n6C5CCC(=O)NC5=O)C4)CC3)nc21]

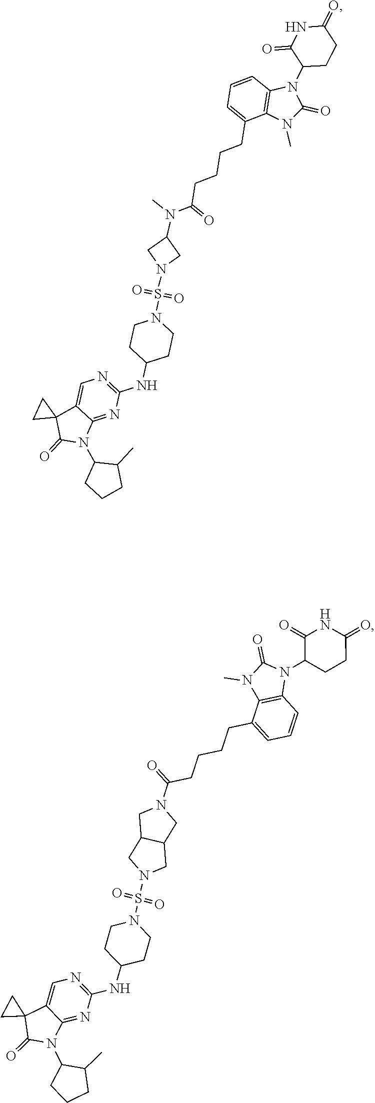 [SMILES: CC1CCCC1N1C(=O)C2(CC2)c2cnc(NC3CCN(S(=O)(=O)N4CC(N(C)C(=O)CCCCc5cccc6c5n(C)c(=O)n6C5CCC(=O)NC5=O)C4)CC3)nc21 ; CC1CCCC1N1C(=O)C2(CC2)c2cnc(NC3CCN(S(=O)(=O)N4CC5CN(C(=O)CCCCc6cccc7c6n(C)c(=O)n7C6CCC(=O)NC6=O)CC5C4)CC3)nc21]

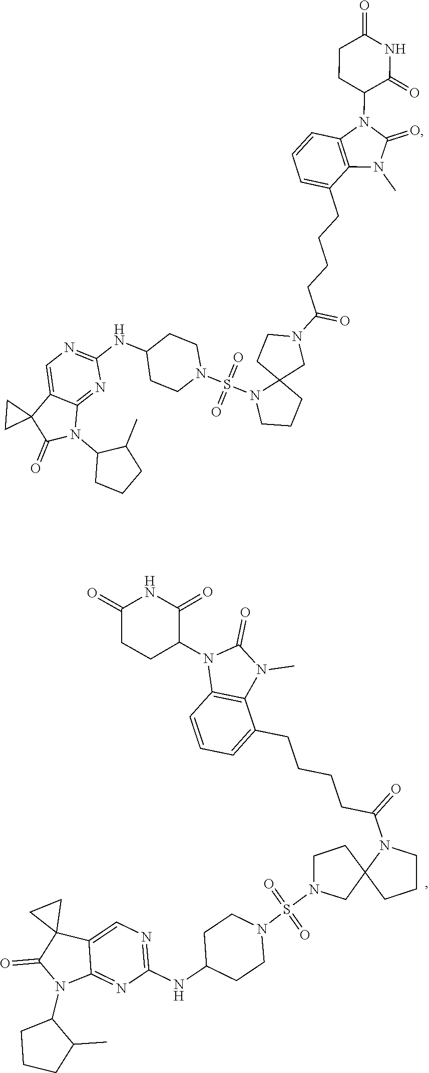 [SMILES: CC1CCCC1N1C(=O)C2(CC2)c2cnc(NC3CCN(S(=O)(=O)N4CCCC45CCN(C(=O)CCCCc4cccc6c4n(C)c(=O)n6C4CCC(=O)NC4=O)C5)CC3)nc21 ; CC1CCCC1N1C(=O)C2(CC2)c2cnc(NC3CCN(S(=O)(=O)N4CCC5(CCCN5C(=O)CCCCc5cccc6c5n(C)c(=O)n6C5CCC(=O)NC5=O)C4)CC3)nc21]

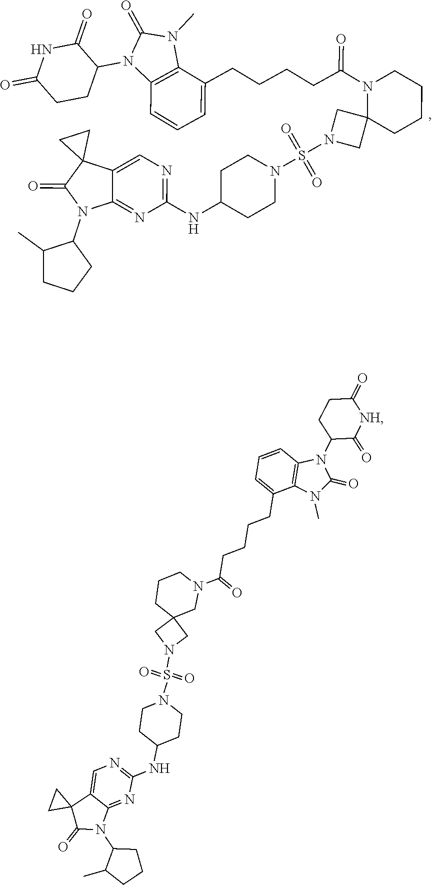 [SMILES: CC1CCCC1N1C(=O)C2(CC2)c2cnc(NC3CCN(S(=O)(=O)N4CC5(CCCCN5C(=O)CCCCc5cccc6c5n(C)c(=O)n6C5CCC(=O)NC5=O)C4)CC3)nc21 ; CC1CCCC1N1C(=O)C2(CC2)c2cnc(NC3CCN(S(=O)(=O)N4CC5(CCCN(C(=O)CCCCc6cccc7c6n(C)c(=O)n7C6CCC(=O)NC6=O)C5)C4)CC3)nc21]

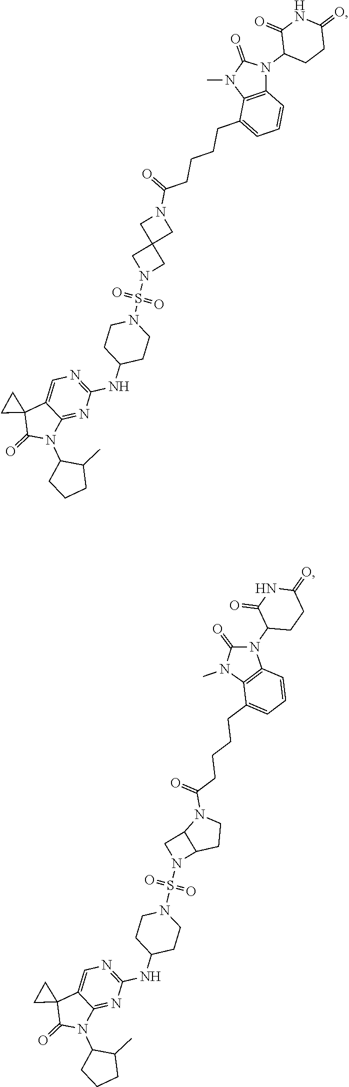 [SMILES: CC1CCCC1N1C(=O)C2(CC2)c2cnc(NC3CCN(S(=O)(=O)N4CC5(CN(C(=O)CCCCc6cccc7c6n(C)c(=O)n7C6CCC(=O)NC6=O)C5)C4)CC3)nc21 ; CC1CCCC1N1C(=O)C2(CC2)c2cnc(NC3CCN(S(=O)(=O)N4CC5C4CCN5C(=O)CCCCc4cccc5c4n(C)c(=O)n5C4CCC(=O)NC4=O)CC3)nc21]

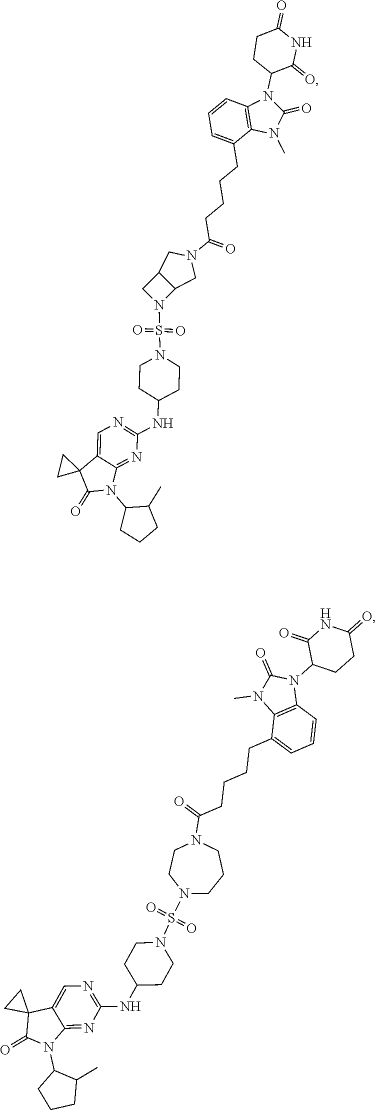 [SMILES: CC1CCCC1N1C(=O)C2(CC2)c2cnc(NC3CCN(S(=O)(=O)N4CC5CN(C(=O)CCCCc6cccc7c6n(C)c(=O)n7C6CCC(=O)NC6=O)CC54)CC3)nc21 ; CC1CCCC1N1C(=O)C2(CC2)c2cnc(NC3CCN(S(=O)(=O)N4CCCN(C(=O)CCCCc5cccc6c5n(C)c(=O)n6C5CCC(=O)NC5=O)CC4)CC3)nc21]

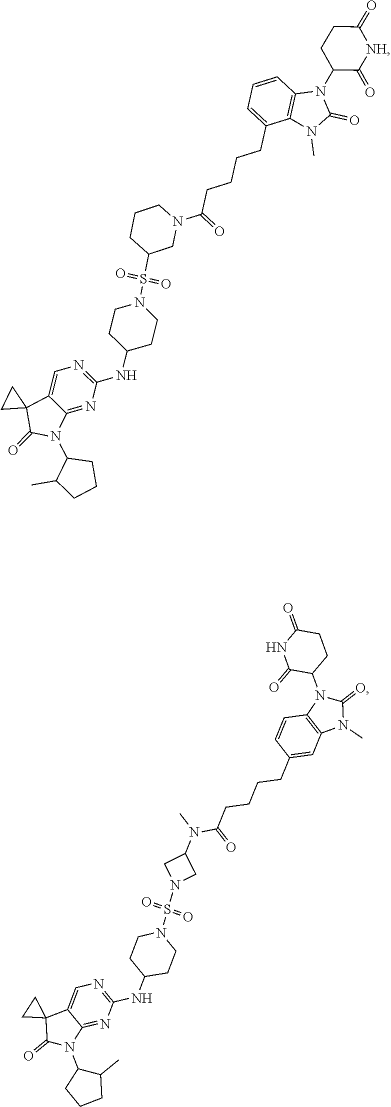 [SMILES: CC1CCCC1N1C(=O)C2(CC2)c2cnc(NC3CCN(S(=O)(=O)C4CCCN(C(=O)CCCCc5cccc6c5n(C)c(=O)n6C5CCC(=O)NC5=O)C4)CC3)nc21 ; CC1CCCC1N1C(=O)C2(CC2)c2cnc(NC3CCN(S(=O)(=O)N4CC(N(C)C(=O)CCCCc5ccc6c(c5)n(C)c(=O)n6C5CCC(=O)NC5=O)C4)CC3)nc21]

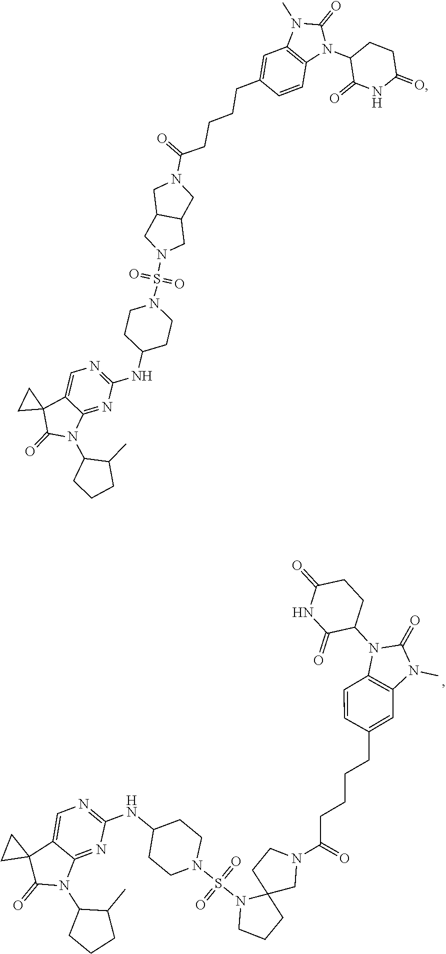 [SMILES: CC1CCCC1N1C(=O)C2(CC2)c2cnc(NC3CCN(S(=O)(=O)N4CC5CN(C(=O)CCCCc6ccc7c(c6)n(C)c(=O)n7C6CCC(=O)NC6=O)CC5C4)CC3)nc21 ; CC1CCCC1N1C(=O)C2(CC2)c2cnc(NC3CCN(S(=O)(=O)N4CCCC45CCN(C(=O)CCCCc4ccc6c(c4)n(C)c(=O)n6C4CCC(=O)NC4=O)C5)CC3)nc21]

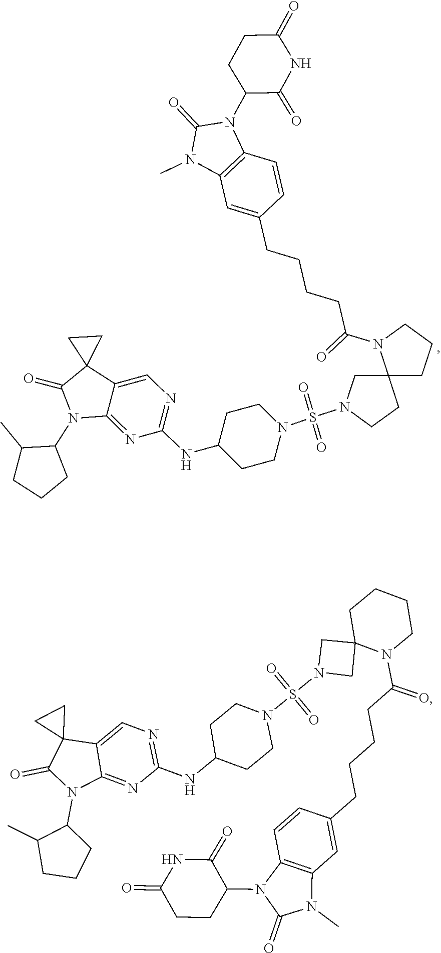 [SMILES: CC1CCCC1N1C(=O)C2(CC2)c2cnc(NC3CCN(S(=O)(=O)N4CCC5(CCCN5C(=O)CCCCc5ccc6c(c5)n(C)c(=O)n6C5CCC(=O)NC5=O)C4)CC3)nc21 ; CC1CCCC1N1C(=O)C2(CC2)c2cnc(NC3CCN(S(=O)(=O)N4CC5(CCCCN5C(=O)CCCCc5ccc6c(c5)n(C)c(=O)n6C5CCC(=O)NC5=O)C4)CC3)nc21]

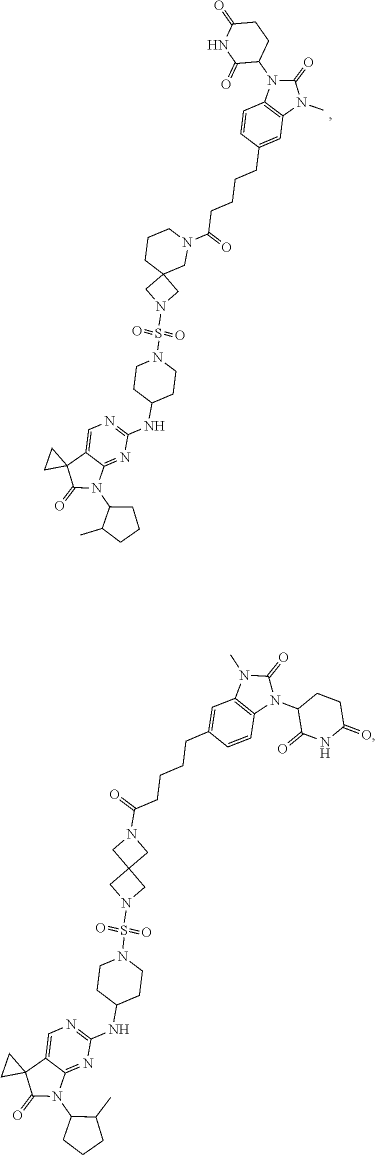 [SMILES: CC1CCCC1N1C(=O)C2(CC2)c2cnc(NC3CCN(S(=O)(=O)N4CC5(CCCN(C(=O)CCCCc6ccc7c(c6)n(C)c(=O)n7C6CCC(=O)NC6=O)C5)C4)CC3)nc21 ; CC1CCCC1N1C(=O)C2(CC2)c2cnc(NC3CCN(S(=O)(=O)N4CC5(CN(C(=O)CCCCc6ccc7c(c6)n(C)c(=O)n7C6CCC(=O)NC6=O)C5)C4)CC3)nc21]

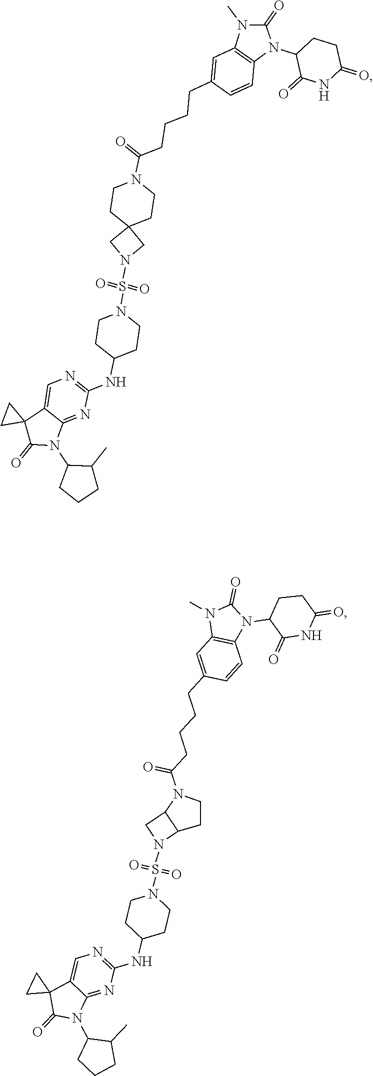 [SMILES: CC1CCCC1N1C(=O)C2(CC2)c2cnc(NC3CCN(S(=O)(=O)N4CC5(CCN(C(=O)CCCCc6ccc7c(c6)n(C)c(=O)n7C6CCC(=O)NC6=O)CC5)C4)CC3)nc21 ; CC1CCCC1N1C(=O)C2(CC2)c2cnc(NC3CCN(S(=O)(=O)N4CC5C4CCN5C(=O)CCCCc4ccc5c(c4)n(C)c(=O)n5C4CCC(=O)NC4=O)CC3)nc21]

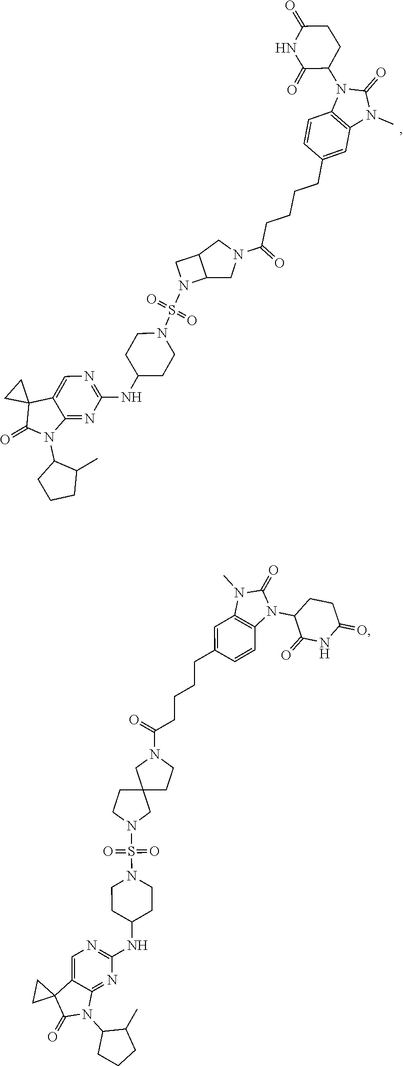 [SMILES: CC1CCCC1N1C(=O)C2(CC2)c2cnc(NC3CCN(S(=O)(=O)N4CC5CN(C(=O)CCCCc6ccc7c(c6)n(C)c(=O)n7C6CCC(=O)NC6=O)CC54)CC3)nc21 ; CC1CCCC1N1C(=O)C2(CC2)c2cnc(NC3CCN(S(=O)(=O)N4CCC5(CCN(C(=O)CCCCc6ccc7c(c6)n(C)c(=O)n7C6CCC(=O)NC6=O)C5)C4)CC3)nc21]

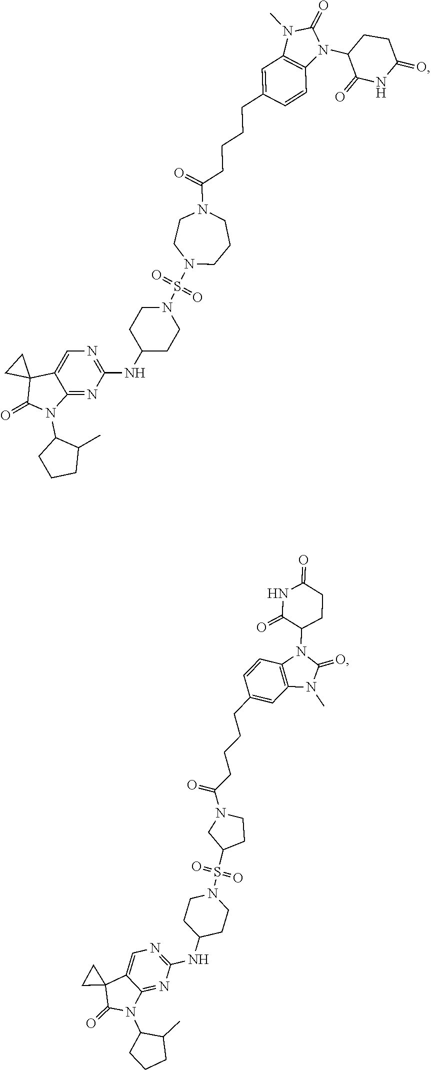 [SMILES: CC1CCCC1N1C(=O)C2(CC2)c2cnc(NC3CCN(S(=O)(=O)N4CCCN(C(=O)CCCCc5ccc6c(c5)n(C)c(=O)n6C5CCC(=O)NC5=O)CC4)CC3)nc21 ; CC1CCCC1N1C(=O)C2(CC2)c2cnc(NC3CCN(S(=O)(=O)C4CCN(C(=O)CCCCc5ccc6c(c5)n(C)c(=O)n6C5CCC(=O)NC5=O)C4)CC3)nc21]

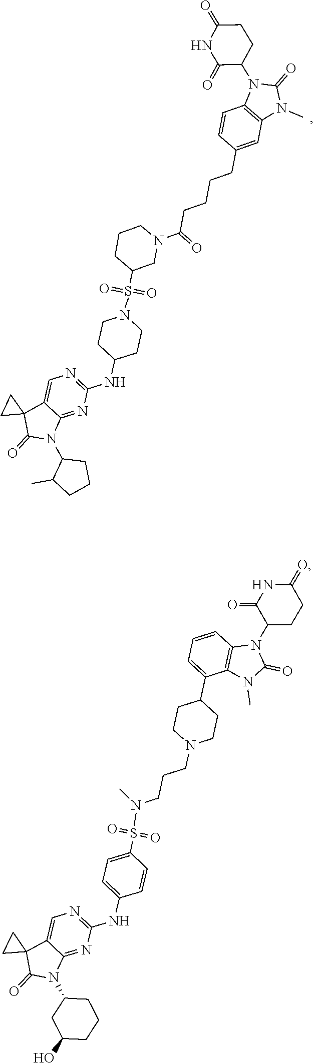 [SMILES: CC1CCCC1N1C(=O)C2(CC2)c2cnc(NC3CCN(S(=O)(=O)C4CCCN(C(=O)CCCCc5ccc6c(c5)n(C)c(=O)n6C5CCC(=O)NC5=O)C4)CC3)nc21 ; CN(CCCN1CCC(c2cccc3c2n(C)c(=O)n3C2CCC(=O)NC2=O)CC1)S(=O)(=O)c1ccc(Nc2ncc3c(n2)N([C@@H]2CCC[C@@H](O)C2)C(=O)C32CC2)cc1]

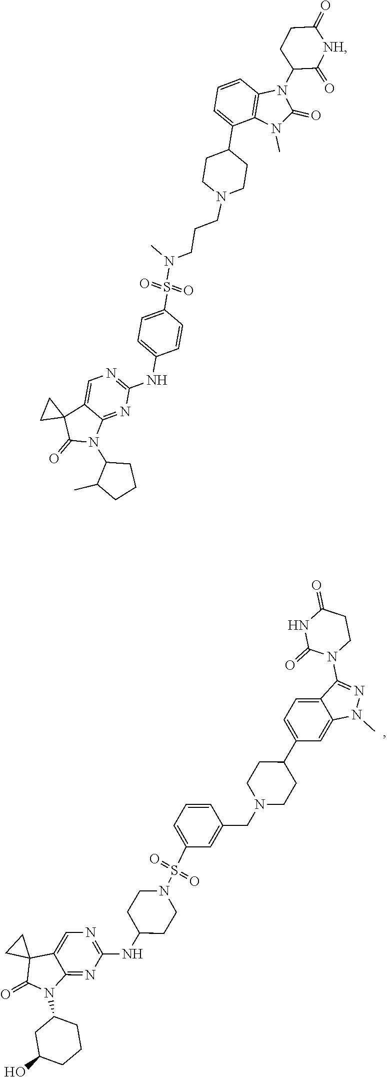 [SMILES: CC1CCCC1N1C(=O)C2(CC2)c2cnc(Nc3ccc(S(=O)(=O)N(C)CCCN4CCC(c5cccc6c5n(C)c(=O)n6C5CCC(=O)NC5=O)CC4)cc3)nc21 ; Cn1nc(N2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)c4)CC3)cc21]

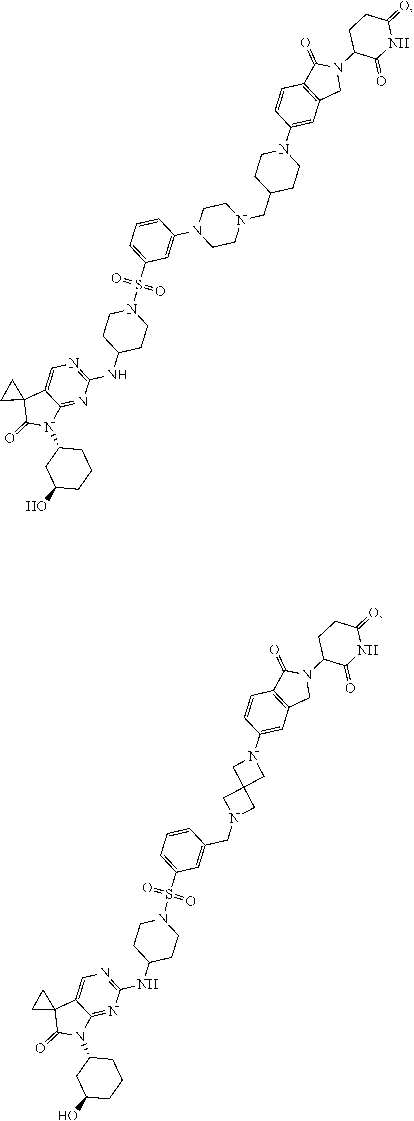 [SMILES: O=C1CCC(N2Cc3cc(N4CCC(CN5CCN(c6cccc(S(=O)(=O)N7CCC(Nc8ncc9c(n8)N([C@@H]8CCC[C@@H](O)C8)C(=O)C98CC8)CC7)c6)CC5)CC4)ccc3C2=O)C(=O)N1 ; O=C1CCC(N2Cc3cc(N4CC5(CN(Cc6cccc(S(=O)(=O)N7CCC(Nc8ncc9c(n8)N([C@@H]8CCC[C@@H](O)C8)C(=O)C98CC8)CC7)c6)C5)C4)ccc3C2=O)C(=O)N1]

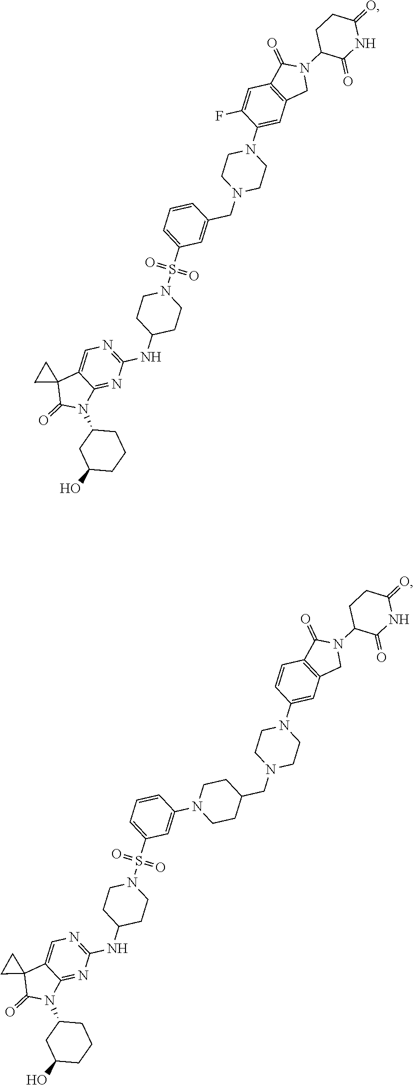 [SMILES: O=C1CCC(N2Cc3cc(N4CCN(Cc5cccc(S(=O)(=O)N6CCC(Nc7ncc8c(n7)N([C@@H]7CCC[C@@H](O)C7)C(=O)C87CC7)CC6)c5)CC4)c(F)cc3C2=O)C(=O)N1 ; O=C1CCC(N2Cc3cc(N4CCN(CC5CCN(c6cccc(S(=O)(=O)N7CCC(Nc8ncc9c(n8)N([C@@H]8CCC[C@@H](O)C8)C(=O)C98CC8)CC7)c6)CC5)CC4)ccc3C2=O)C(=O)N1]

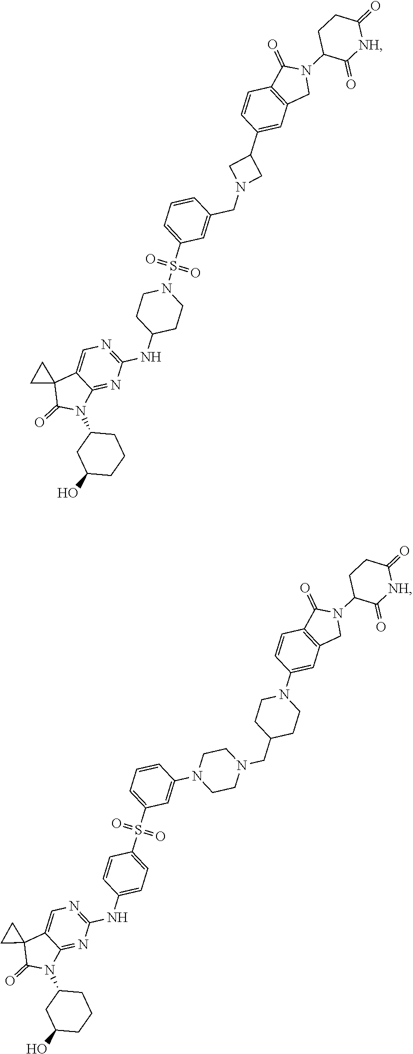 [SMILES: O=C1CCC(N2Cc3cc(C4CN(Cc5cccc(S(=O)(=O)N6CCC(Nc7ncc8c(n7)N([C@@H]7CCC[C@@H](O)C7)C(=O)C87CC7)CC6)c5)C4)ccc3C2=O)C(=O)N1 ; O=C1CCC(N2Cc3cc(N4CCC(CN5CCN(c6cccc(S(=O)(=O)c7ccc(Nc8ncc9c(n8)N([C@@H]8CCC[C@@H](O)C8)C(=O)C98CC8)cc7)c6)CC5)CC4)ccc3C2=O)C(=O)N1]

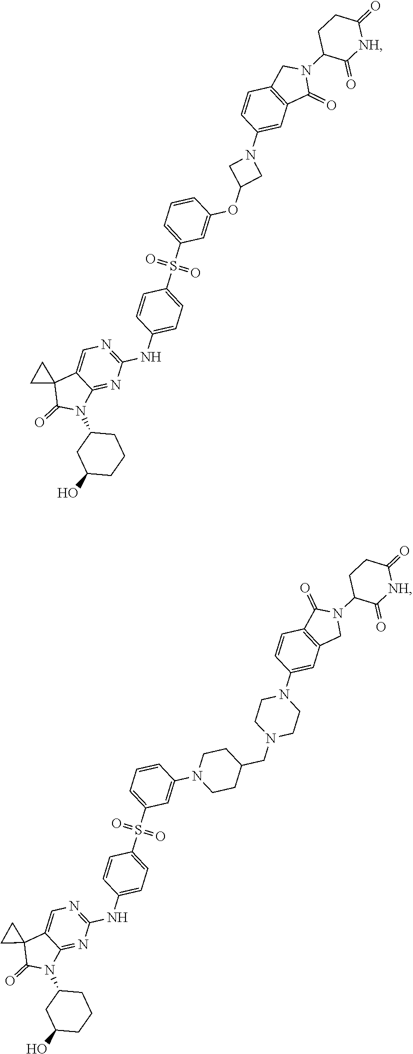 [SMILES: O=C1CCC(N2Cc3ccc(N4CC(Oc5cccc(S(=O)(=O)c6ccc(Nc7ncc8c(n7)N([C@@H]7CCC[C@@H](O)C7)C(=O)C87CC7)cc6)c5)C4)cc3C2=O)C(=O)N1 ; O=C1CCC(N2Cc3cc(N4CCN(CC5CCN(c6cccc(S(=O)(=O)c7ccc(Nc8ncc9c(n8)N([C@@H]8CCC[C@@H](O)C8)C(=O)C98CC8)cc7)c6)CC5)CC4)ccc3C2=O)C(=O)N1]

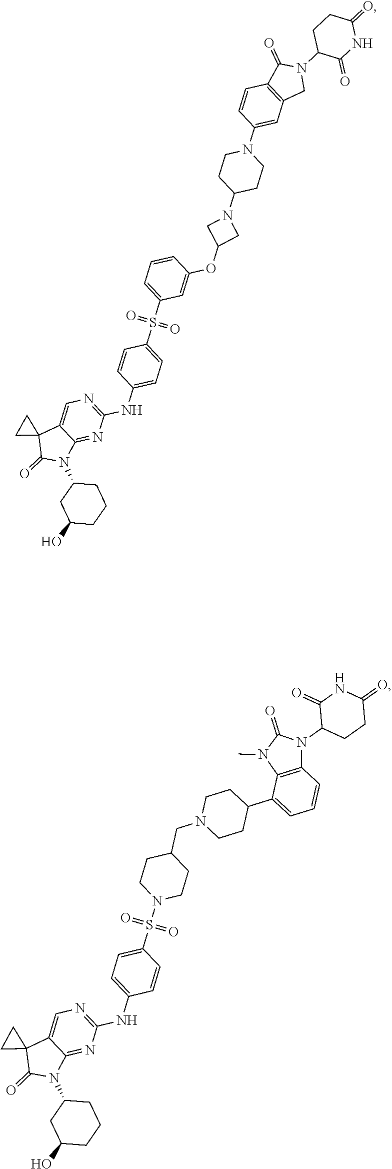 [SMILES: O=C1CCC(N2Cc3cc(N4CCC(N5CC(Oc6cccc(S(=O)(=O)c7ccc(Nc8ncc9c(n8)N([C@@H]8CCC[C@@H](O)C8)C(=O)C98CC8)cc7)c6)C5)CC4)ccc3C2=O)C(=O)N1 ; Cn1c(=O)n(C2CCC(=O)NC2=O)c2cccc(C3CCN(CC4CCN(S(=O)(=O)c5ccc(Nc6ncc7c(n6)N([C@@H]6CCC[C@@H](O)C6)C(=O)C76CC6)cc5)CC4)CC3)c21]

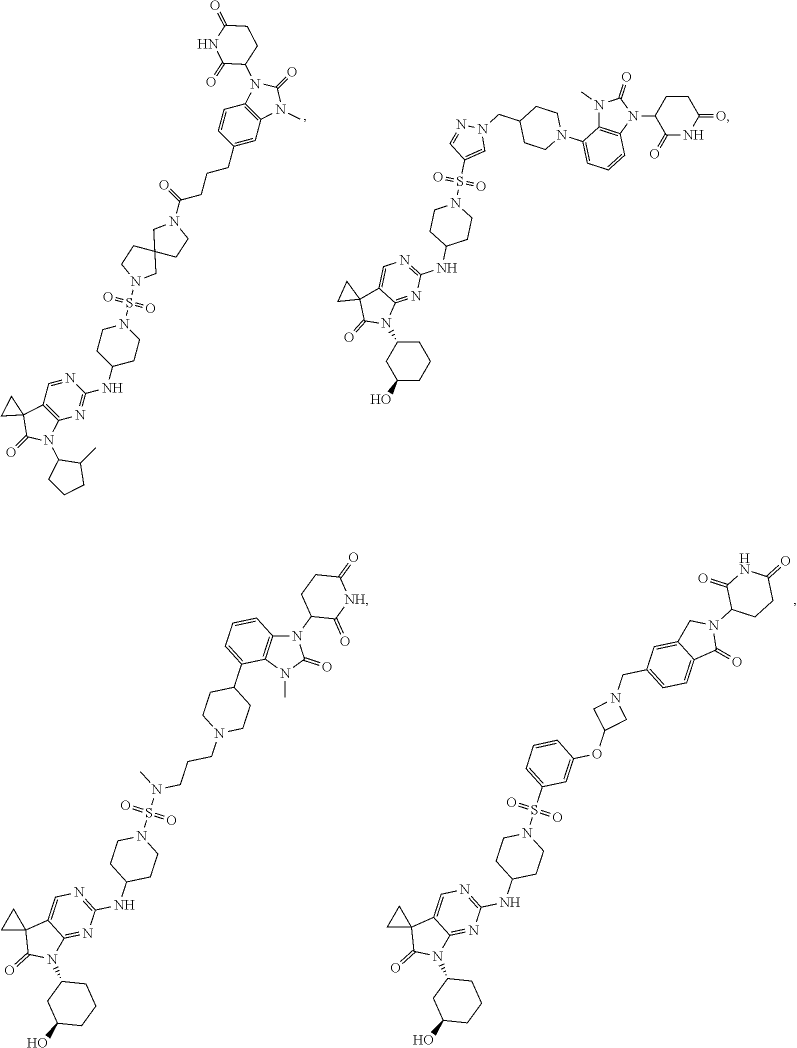 [SMILES: CC1CCCC1N1C(=O)C2(CC2)c2cnc(NC3CCN(S(=O)(=O)N4CCC5(CCN(C(=O)CCCc6ccc7c(c6)n(C)c(=O)n7C6CCC(=O)NC6=O)C5)C4)CC3)nc21 ; Cn1c(=O)n(C2CCC(=O)NC2=O)c2cccc(N3CCC(Cn4cc(S(=O)(=O)N5CCC(Nc6ncc7c(n6)N([C@@H]6CCC[C@@H](O)C6)C(=O)C76CC6)CC5)cn4)CC3)c21 ; CN(CCCN1CCC(c2cccc3c2n(C)c(=O)n3C2CCC(=O)NC2=O)CC1)S(=O)(=O)N1CCC(Nc2ncc3c(n2)N([C@@H]2CCC[C@@H](O)C2)C(=O)C32CC2)CC1 ; O=C1CCC(N2Cc3cc(CN4CC(Oc5cccc(S(=O)(=O)N6CCC(Nc7ncc8c(n7)N([C@@H]7CCC[C@@H](O)C7)C(=O)C87CC7)CC6)c5)C4)ccc3C2=O)C(=O)N1]

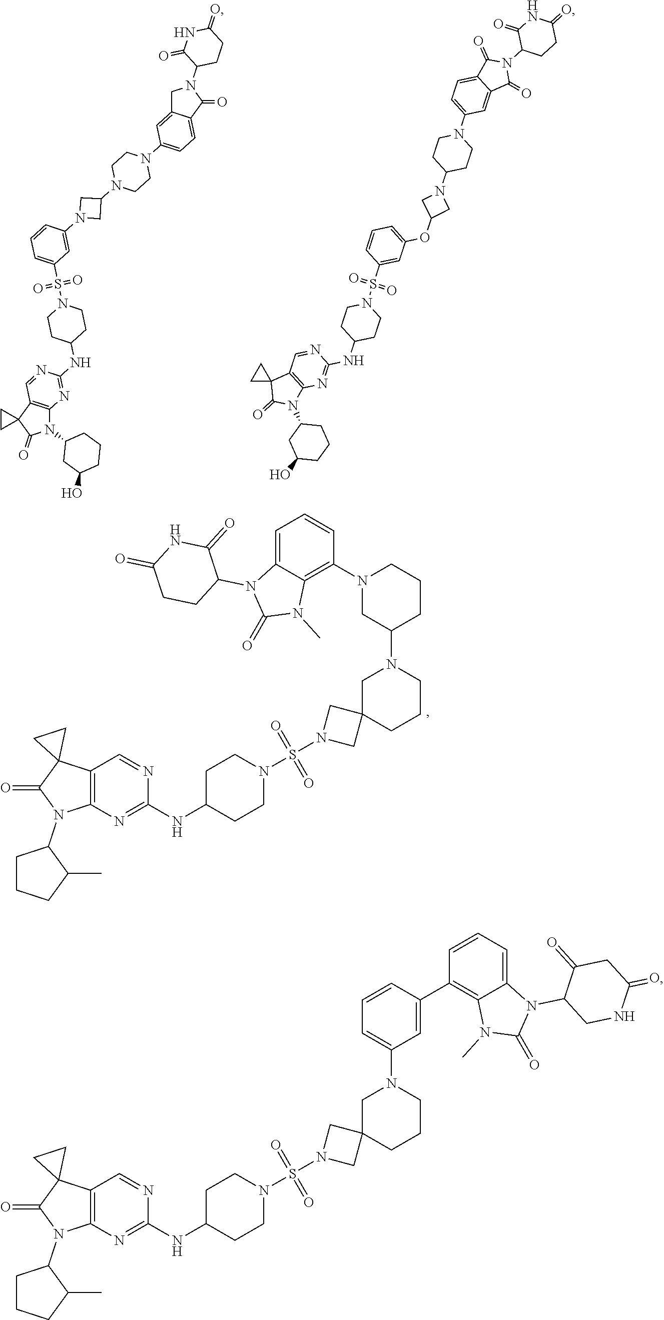 [SMILES: O=C1CCC(N2Cc3cc(N4CCN(C5CN(c6cccc(S(=O)(=O)N7CCC(Nc8ncc9c(n8)N([C@@H]8CCC[C@@H](O)C8)C(=O)C98CC8)CC7)c6)C5)CC4)ccc3C2=O)C(=O)N1 ; O=C1CCC(N2C(=O)c3ccc(N4CCC(N5CC(Oc6cccc(S(=O)(=O)N7CCC(Nc8ncc9c(n8)N([C@@H]8CCC[C@@H](O)C8)C(=O)C98CC8)CC7)c6)C5)CC4)cc3C2=O)C(=O)N1 ; CC1CCCC1N1C(=O)C2(CC2)c2cnc(NC3CCN(S(=O)(=O)N4CC5(CCCN(C6CCCN(c7cccc8c7n(C)c(=O)n8C7CCC(=O)NC7=O)C6)C5)C4)CC3)nc21 ; CC1CCCC1N1C(=O)C2(CC2)c2cnc(NC3CCN(S(=O)(=O)N4CC5(CCCN(c6cccc(-c7cccc8c7n(C)c(=O)n8C7CNC(=O)CC7=O)c6)C5)C4)CC3)nc21]

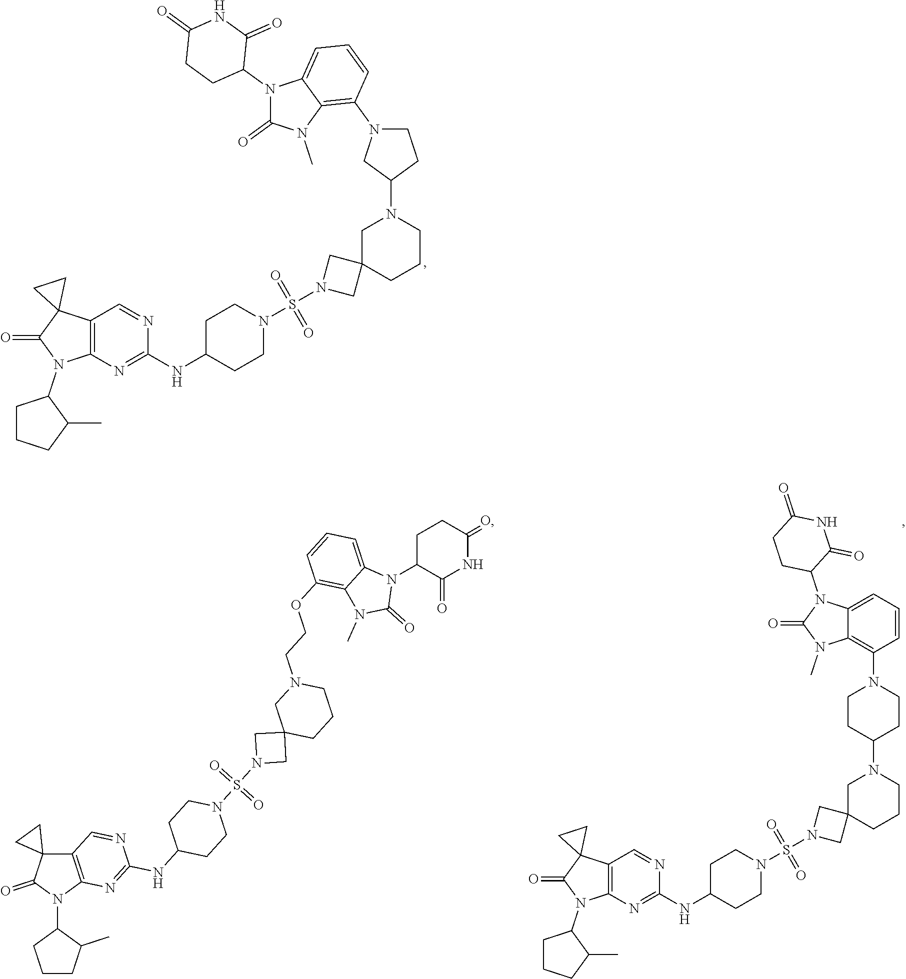 [SMILES: CC1CCCC1N1C(=O)C2(CC2)c2cnc(NC3CCN(S(=O)(=O)N4CC5(CCCN(C6CCN(c7cccc8c7n(C)c(=O)n8C7CCC(=O)NC7=O)C6)C5)C4)CC3)nc21 ; CC1CCCC1N1C(=O)C2(CC2)c2cnc(NC3CCN(S(=O)(=O)N4CC5(CCCN(CCOc6cccc7c6n(C)c(=O)n7C6CCC(=O)NC6=O)C5)C4)CC3)nc21 ; CC1CCCC1N1C(=O)C2(CC2)c2cnc(NC3CCN(S(=O)(=O)N4CC5(CCCN(C6CCN(c7cccc8c7n(C)c(=O)n8C7CCC(=O)NC7=O)CC6)C5)C4)CC3)nc21]

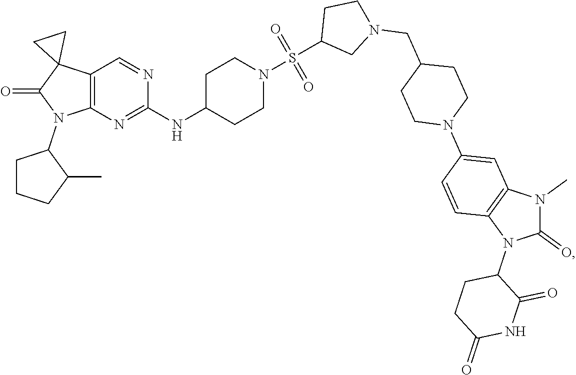 [SMILES: CC1CCCC1N1C(=O)C2(CC2)c2cnc(NC3CCN(S(=O)(=O)C4CCN(CC5CCN(c6ccc7c(c6)n(C)c(=O)n7C6CCC(=O)NC6=O)CC5)C4)CC3)nc21]

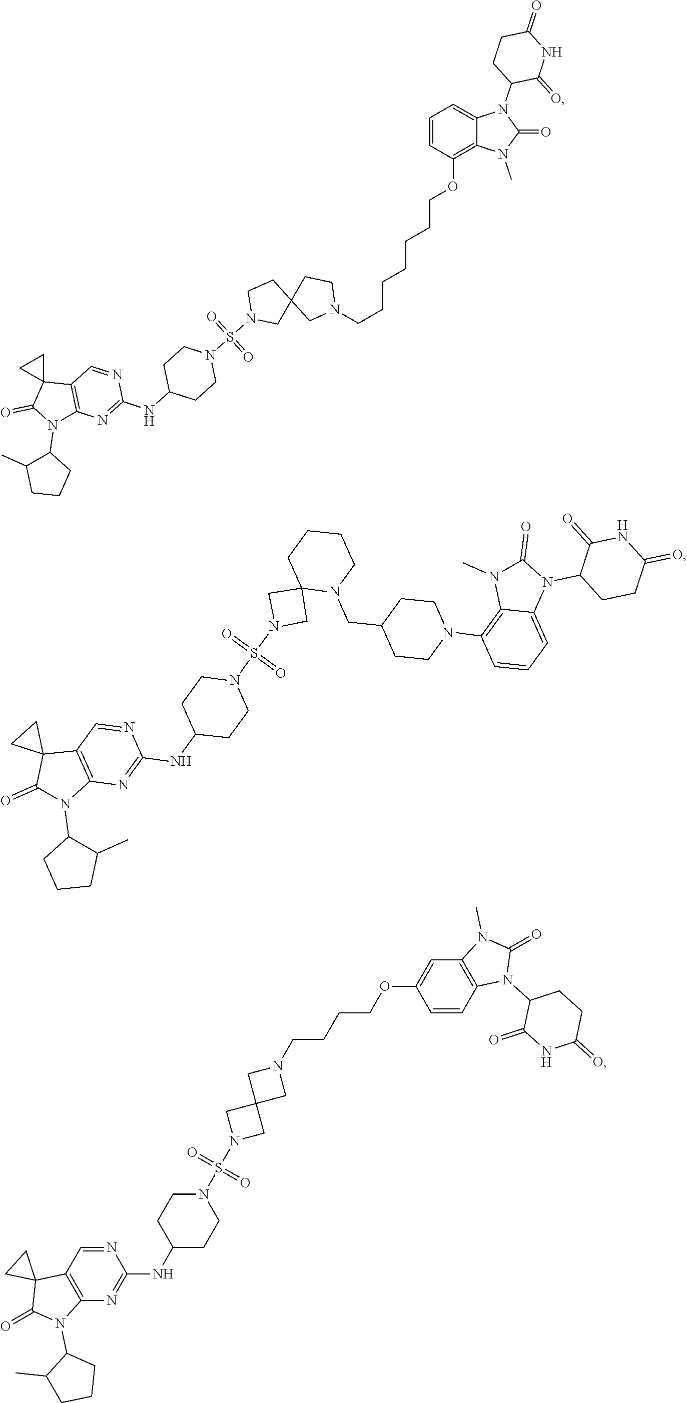 [SMILES: CC1CCCC1N1C(=O)C2(CC2)c2cnc(NC3CCN(S(=O)(=O)N4CCC5(CCN(CCCCCCCOc6cccc7c6n(C)c(=O)n7C6CCC(=O)NC6=O)C5)C4)CC3)nc21 ; CC1CCCC1N1C(=O)C2(CC2)c2cnc(NC3CCN(S(=O)(=O)N4CC5(CCCCN5CC5CCN(c6cccc7c6n(C)c(=O)n7C6CCC(=O)NC6=O)CC5)C4)CC3)nc21 ; CC1CCCC1N1C(=O)C2(CC2)c2cnc(NC3CCN(S(=O)(=O)N4CC5(CN(CCCCOc6ccc7c(c6)n(C)c(=O)n7C6CCC(=O)NC6=O)C5)C4)CC3)nc21]

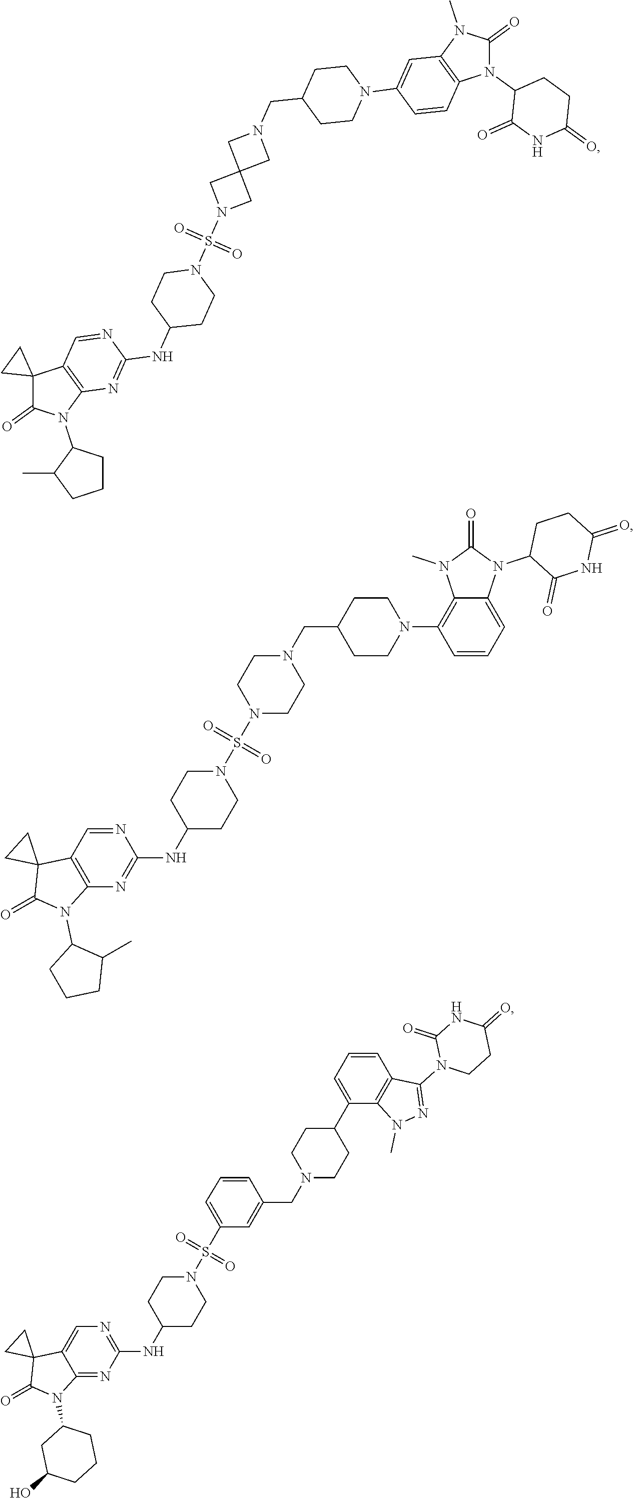 [SMILES: CC1CCCC1N1C(=O)C2(CC2)c2cnc(NC3CCN(S(=O)(=O)N4CC5(CN(CC6CCN(c7ccc8c(c7)n(C)c(=O)n8C7CCC(=O)NC7=O)CC6)C5)C4)CC3)nc21 ; CC1CCCC1N1C(=O)C2(CC2)c2cnc(NC3CCN(S(=O)(=O)N4CCN(CC5CCN(c6cccc7c6n(C)c(=O)n7C6CCC(=O)NC6=O)CC5)CC4)CC3)nc21 ; Cn1nc(N2CCC(=O)NC2=O)c2cccc(C3CCN(Cc4cccc(S(=O)(=O)N5CCC(Nc6ncc7c(n6)N([C@@H]6CCC[C@@H](O)C6)C(=O)C76CC6)CC5)c4)CC3)c21]

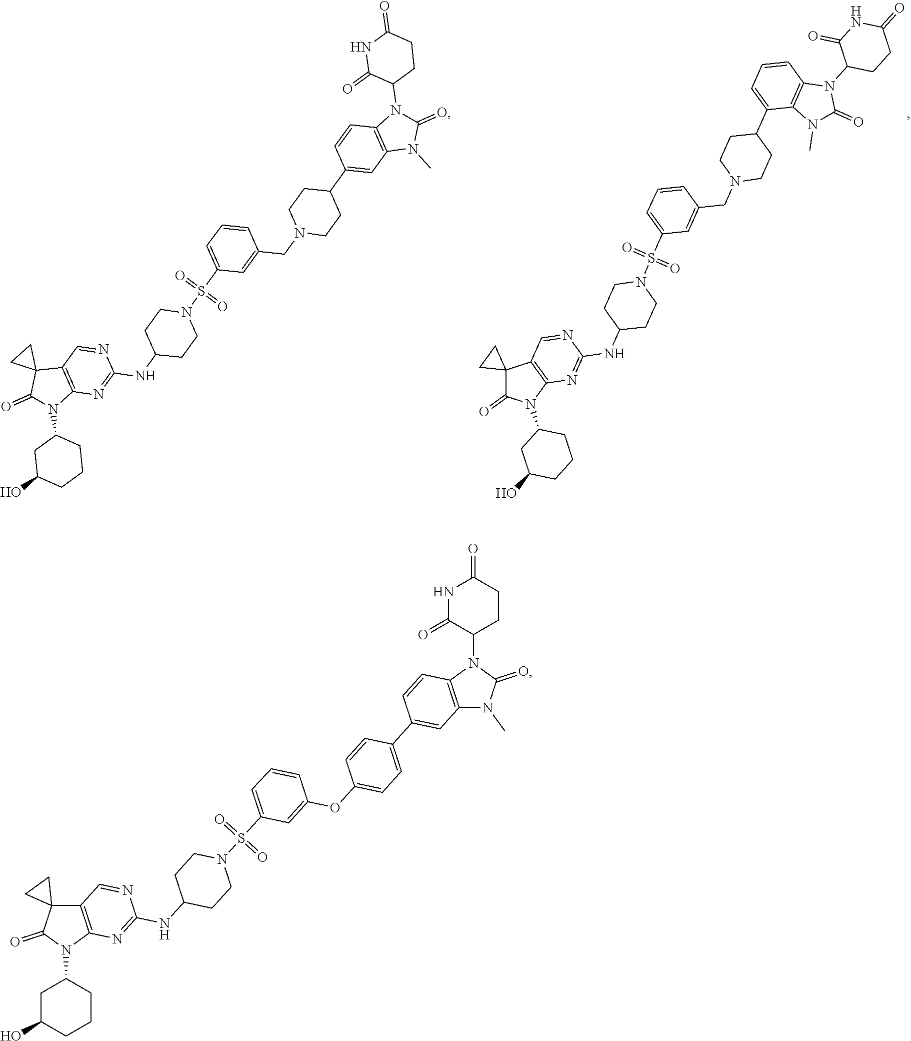 [SMILES: Cn1c(=O)n(C2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)c4)CC3)cc21 ; Cn1c(=O)n(C2CCC(=O)NC2=O)c2cccc(C3CCN(Cc4cccc(S(=O)(=O)N5CCC(Nc6ncc7c(n6)N([C@@H]6CCC[C@@H](O)C6)C(=O)C76CC6)CC5)c4)CC3)c21 ; Cn1c(=O)n(C2CCC(=O)NC2=O)c2ccc(-c3ccc(Oc4cccc(S(=O)(=O)N5CCC(Nc6ncc7c(n6)N([C@@H]6CCC[C@@H](O)C6)C(=O)C76CC6)CC5)c4)cc3)cc21]

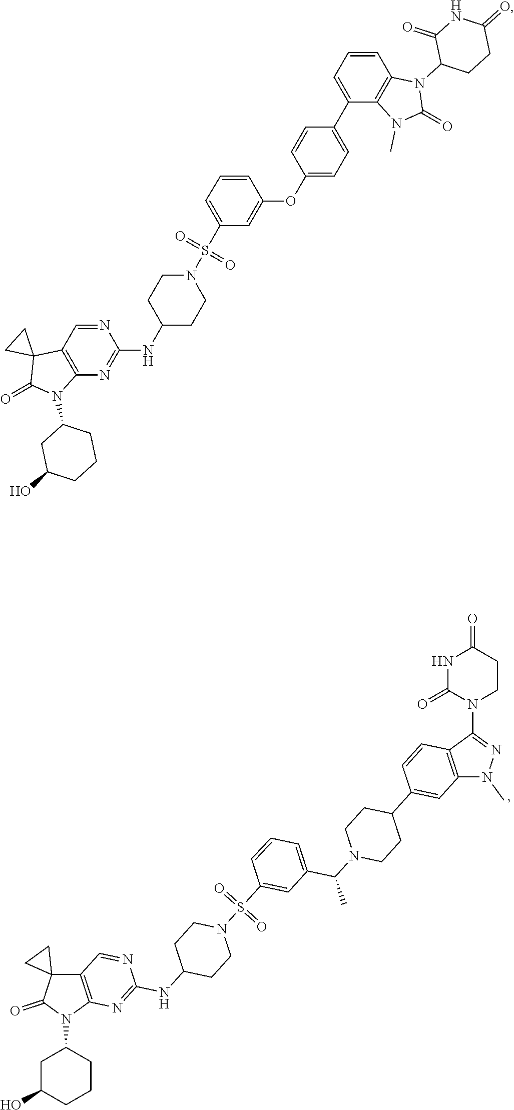 [SMILES: Cn1c(=O)n(C2CCC(=O)NC2=O)c2cccc(-c3ccc(Oc4cccc(S(=O)(=O)N5CCC(Nc6ncc7c(n6)N([C@@H]6CCC[C@@H](O)C6)C(=O)C76CC6)CC5)c4)cc3)c21 ; C[C@H](c1cccc(S(=O)(=O)N2CCC(Nc3ncc4c(n3)N([C@@H]3CCC[C@@H](O)C3)C(=O)C43CC3)CC2)c1)N1CCC(c2ccc3c(N4CCC(=O)NC4=O)nn(C)c3c2)CC1]

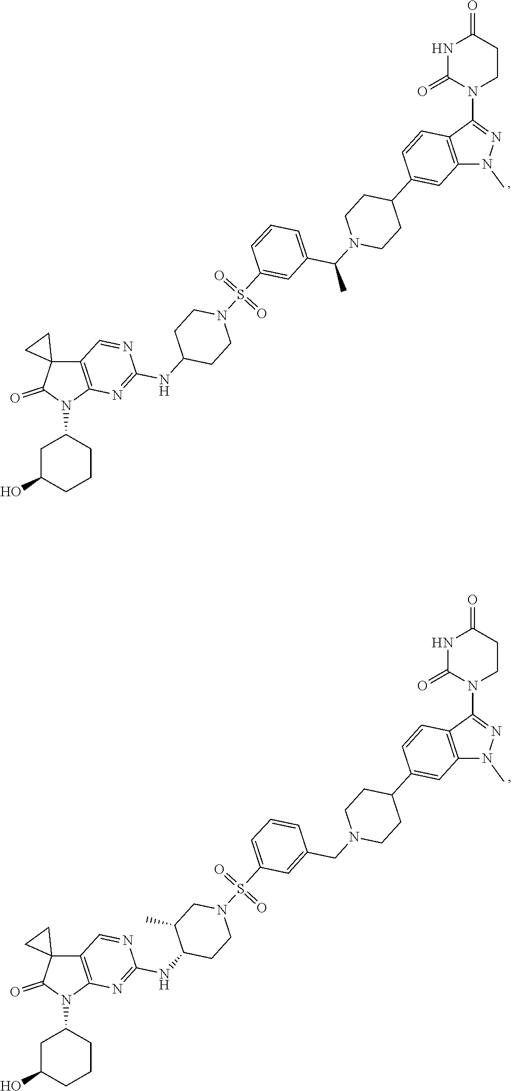 [SMILES: C[C@@H](c1cccc(S(=O)(=O)N2CCC(Nc3ncc4c(n3)N([C@@H]3CCC[C@@H](O)C3)C(=O)C43CC3)CC2)c1)N1CCC(c2ccc3c(N4CCC(=O)NC4=O)nn(C)c3c2)CC1 ; C[C@@H]1CN(S(=O)(=O)c2cccc(CN3CCC(c4ccc5c(N6CCC(=O)NC6=O)nn(C)c5c4)CC3)c2)CC[C@@H]1Nc1ncc2c(n1)N([C@@H]1CCC[C@@H](O)C1)C(=O)C21CC1]

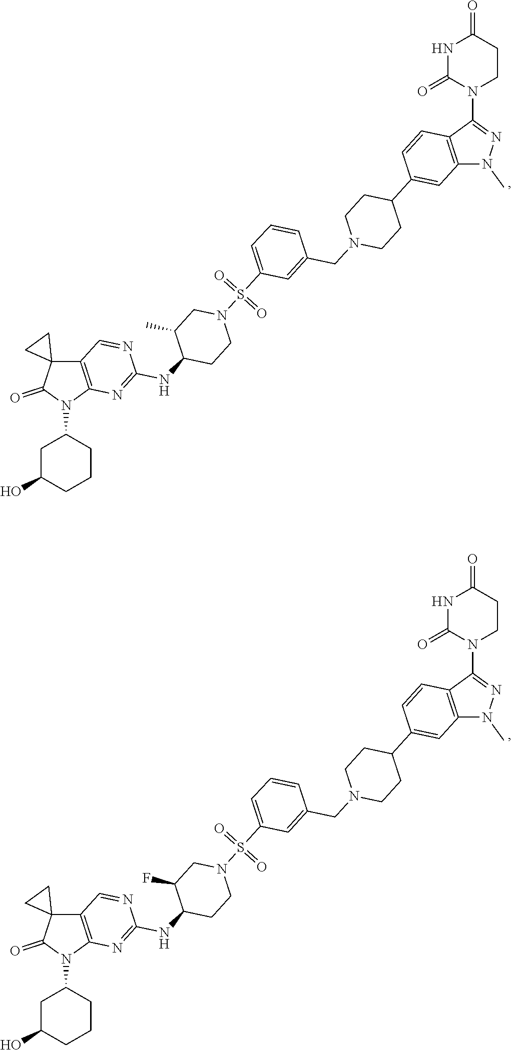 [SMILES: C[C@@H]1CN(S(=O)(=O)c2cccc(CN3CCC(c4ccc5c(N6CCC(=O)NC6=O)nn(C)c5c4)CC3)c2)CC[C@H]1Nc1ncc2c(n1)N([C@@H]1CCC[C@@H](O)C1)C(=O)C21CC1 ; Cn1nc(N2CCC(=O)NC2=O)c2ccc(C3CCN(Cc4cccc(S(=O)(=O)N5CC[C@@H](Nc6ncc7c(n6)N([C@@H]6CCC[C@@H](O)C6)C(=O)C76CC6)[C@@H](F)C5)c4)CC3)cc21]

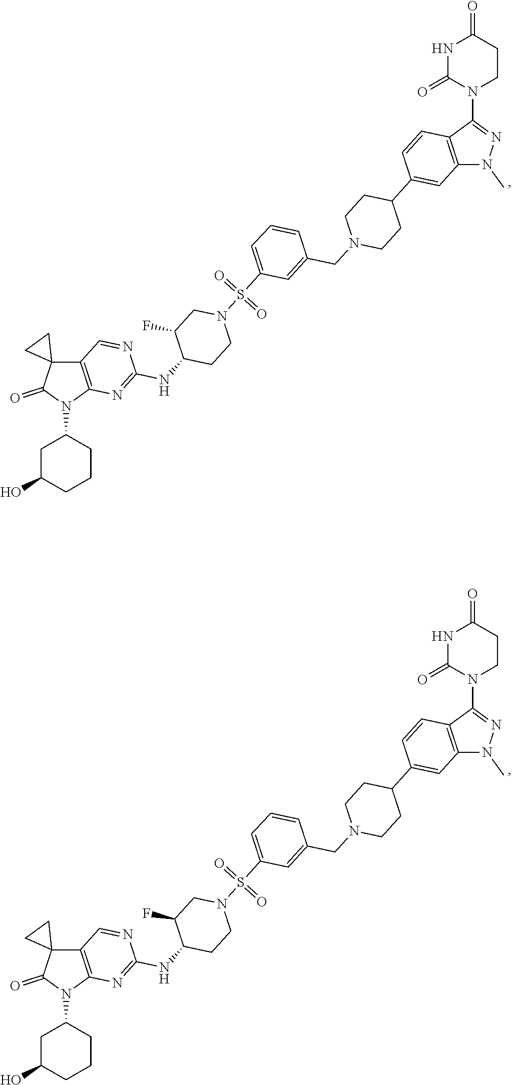 [SMILES: Cn1nc(N2CCC(=O)NC2=O)c2ccc(C3CCN(Cc4cccc(S(=O)(=O)N5CC[C@H](Nc6ncc7c(n6)N([C@@H]6CCC[C@@H](O)C6)C(=O)C76CC6)[C@H](F)C5)c4)CC3)cc21 ; Cn1nc(N2CCC(=O)NC2=O)c2ccc(C3CCN(Cc4cccc(S(=O)(=O)N5CC[C@H](Nc6ncc7c(n6)N([C@@H]6CCC[C@@H](O)C6)C(=O)C76CC6)[C@@H](F)C5)c4)CC3)cc21]

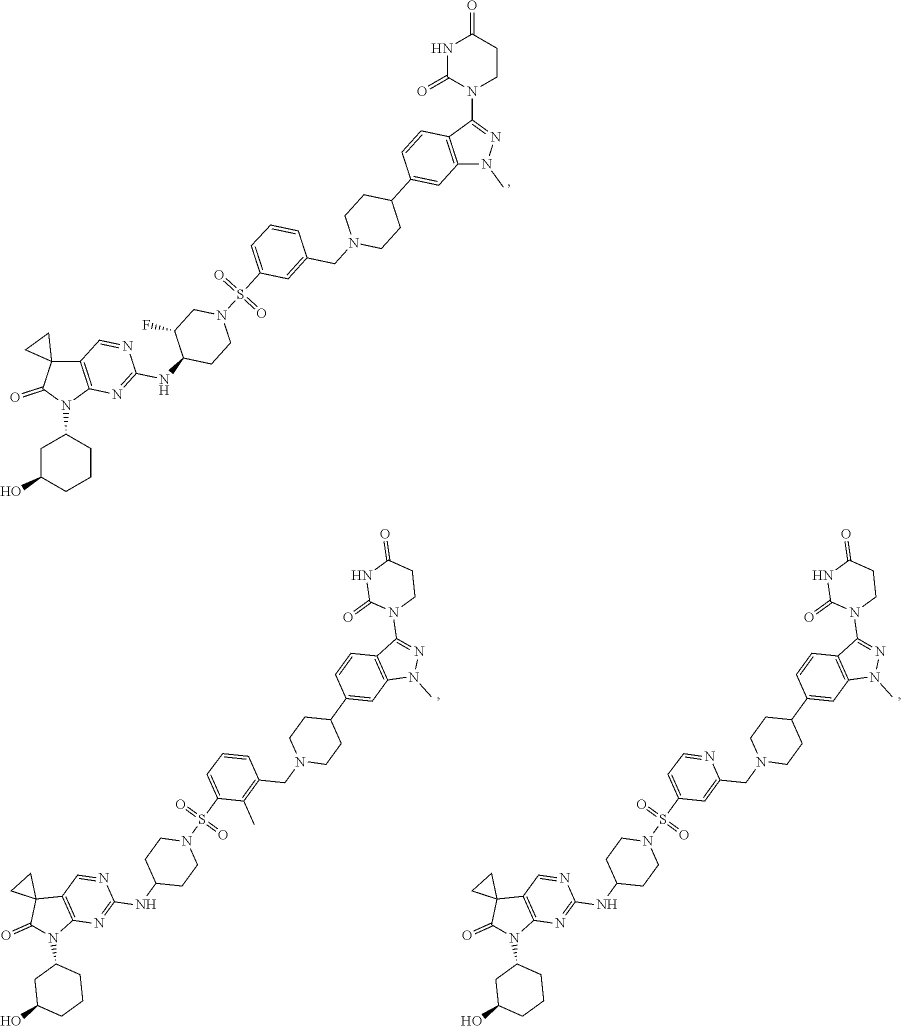 [SMILES: Cn1nc(N2CCC(=O)NC2=O)c2ccc(C3CCN(Cc4cccc(S(=O)(=O)N5CC[C@@H](Nc6ncc7c(n6)N([C@@H]6CCC[C@@H](O)C6)C(=O)C76CC6)[C@H](F)C5)c4)CC3)cc21 ; Cc1c(CN2CCC(c3ccc4c(N5CCC(=O)NC5=O)nn(C)c4c3)CC2)cccc1S(=O)(=O)N1CCC(Nc2ncc3c(n2)N([C@@H]2CCC[C@@H](O)C2)C(=O)C32CC2)CC1 ; Cn1nc(N2CCC(=O)NC2=O)c2ccc(C3CCN(Cc4cc(S(=O)(=O)N5CCC(Nc6ncc7c(n6)N([C@@H]6CCC[C@@H](O)C6)C(=O)C76CC6)CC5)ccn4)CC3)cc21]

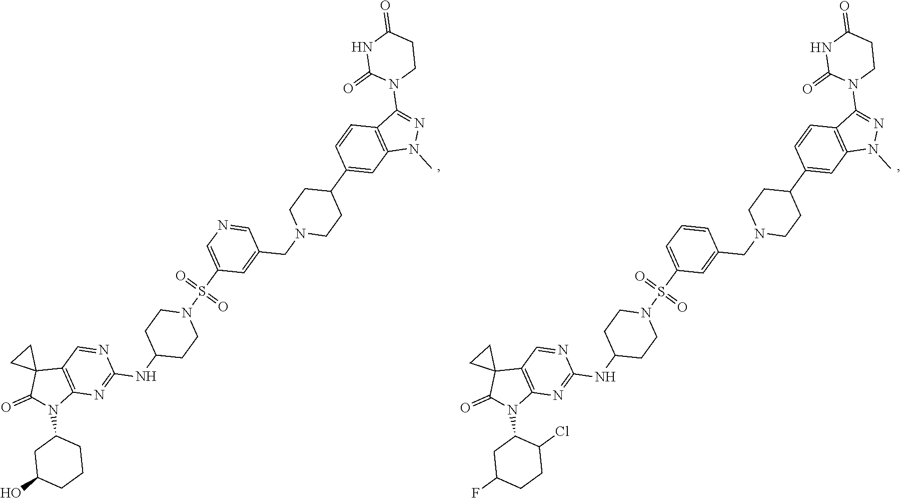 [SMILES: Cn1nc(N2CCC(=O)NC2=O)c2ccc(C3CCN(Cc4cncc(S(=O)(=O)N5CCC(Nc6ncc7c(n6)N([C@@H]6CCC[C@@H](O)C6)C(=O)C76CC6)CC5)c4)CC3)cc21 ; Cn1nc(N2CCC(=O)NC2=O)c2ccc(C3CCN(Cc4cccc(S(=O)(=O)N5CCC(Nc6ncc7c(n6)N([C@H]6CC(F)CCC6Cl)C(=O)C76CC6)CC5)c4)CC3)cc21]

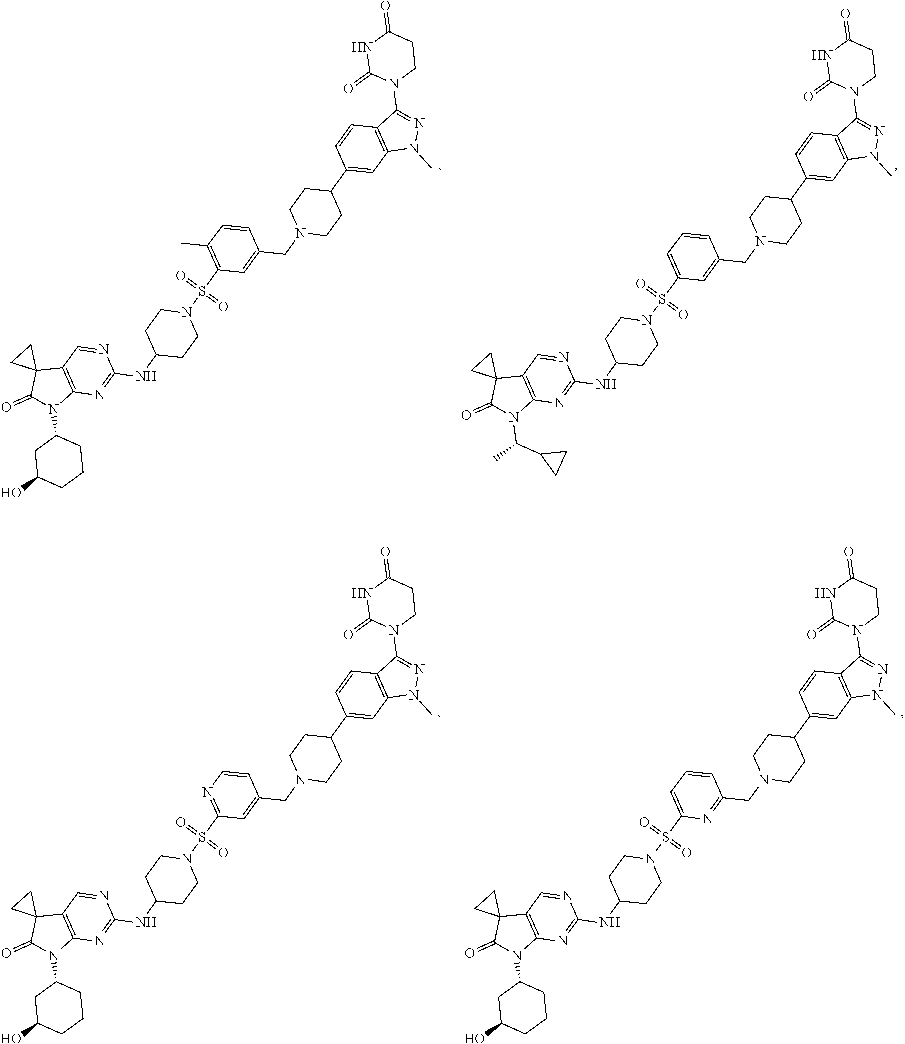 [SMILES: Cc1ccc(CN2CCC(c3ccc4c(N5CCC(=O)NC5=O)nn(C)c4c3)CC2)cc1S(=O)(=O)N1CCC(Nc2ncc3c(n2)N([C@@H]2CCC[C@@H](O)C2)C(=O)C32CC2)CC1 ; C[C@@H](C1CC1)N1C(=O)C2(CC2)c2cnc(NC3CCN(S(=O)(=O)c4cccc(CN5CCC(c6ccc7c(N8CCC(=O)NC8=O)nn(C)c7c6)CC5)c4)CC3)nc21 ; Cn1nc(N2CCC(=O)NC2=O)c2ccc(C3CCN(Cc4ccnc(S(=O)(=O)N5CCC(Nc6ncc7c(n6)N([C@@H]6CCC[C@@H](O)C6)C(=O)C76CC6)CC5)c4)CC3)cc21 ; Cn1nc(N2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)n4)CC3)cc21]

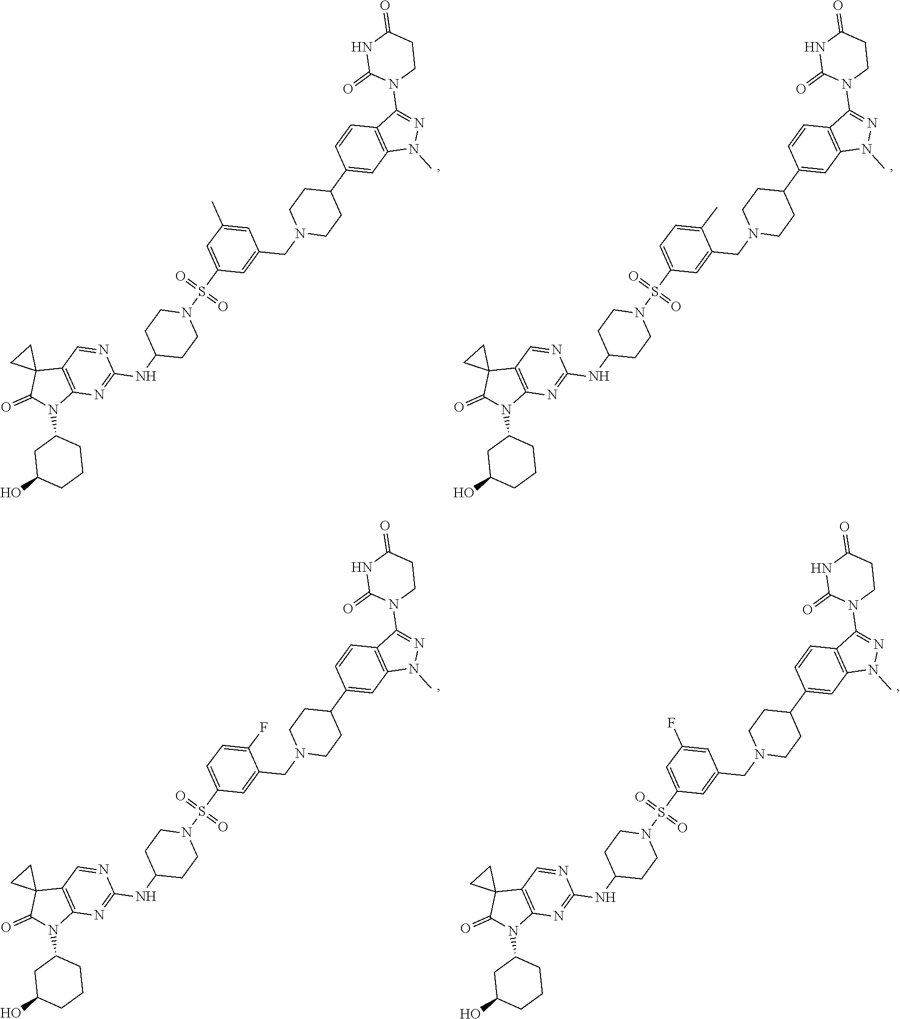 [SMILES: Cc1cc(CN2CCC(c3ccc4c(N5CCC(=O)NC5=O)nn(C)c4c3)CC2)cc(S(=O)(=O)N2CCC(Nc3ncc4c(n3)N([C@@H]3CCC[C@@H](O)C3)C(=O)C43CC3)CC2)c1 ; Cc1ccc(S(=O)(=O)N2CCC(Nc3ncc4c(n3)N([C@@H]3CCC[C@@H](O)C3)C(=O)C43CC3)CC2)cc1CN1CCC(c2ccc3c(N4CCC(=O)NC4=O)nn(C)c3c2)CC1 ; Cn1nc(N2CCC(=O)NC2=O)c2ccc(C3CCN(Cc4cc(S(=O)(=O)N5CCC(Nc6ncc7c(n6)N([C@@H]6CCC[C@@H](O)C6)C(=O)C76CC6)CC5)ccc4F)CC3)cc21 ; Cn1nc(N2CCC(=O)NC2=O)c2ccc(C3CCN(Cc4cc(F)cc(S(=O)(=O)N5CCC(Nc6ncc7c(n6)N([C@@H]6CCC[C@@H](O)C6)C(=O)C76CC6)CC5)c4)CC3)cc21]

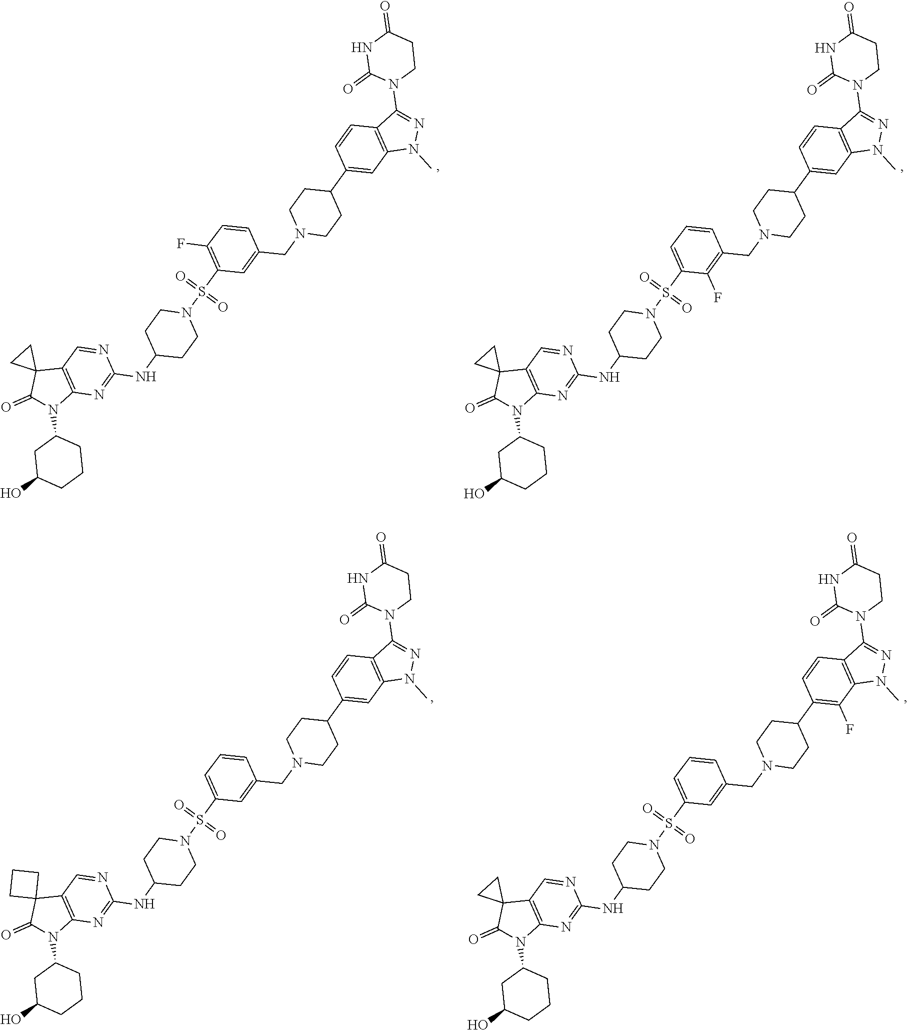 [SMILES: Cn1nc(N2CCC(=O)NC2=O)c2ccc(C3CCN(Cc4ccc(F)c(S(=O)(=O)N5CCC(Nc6ncc7c(n6)N([C@@H]6CCC[C@@H](O)C6)C(=O)C76CC6)CC5)c4)CC3)cc21 ; Cn1nc(N2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)c4F)CC3)cc21 ; Cn1nc(N2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CCC6)CC5)c4)CC3)cc21 ; Cn1nc(N2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)c4)CC3)c(F)c21]

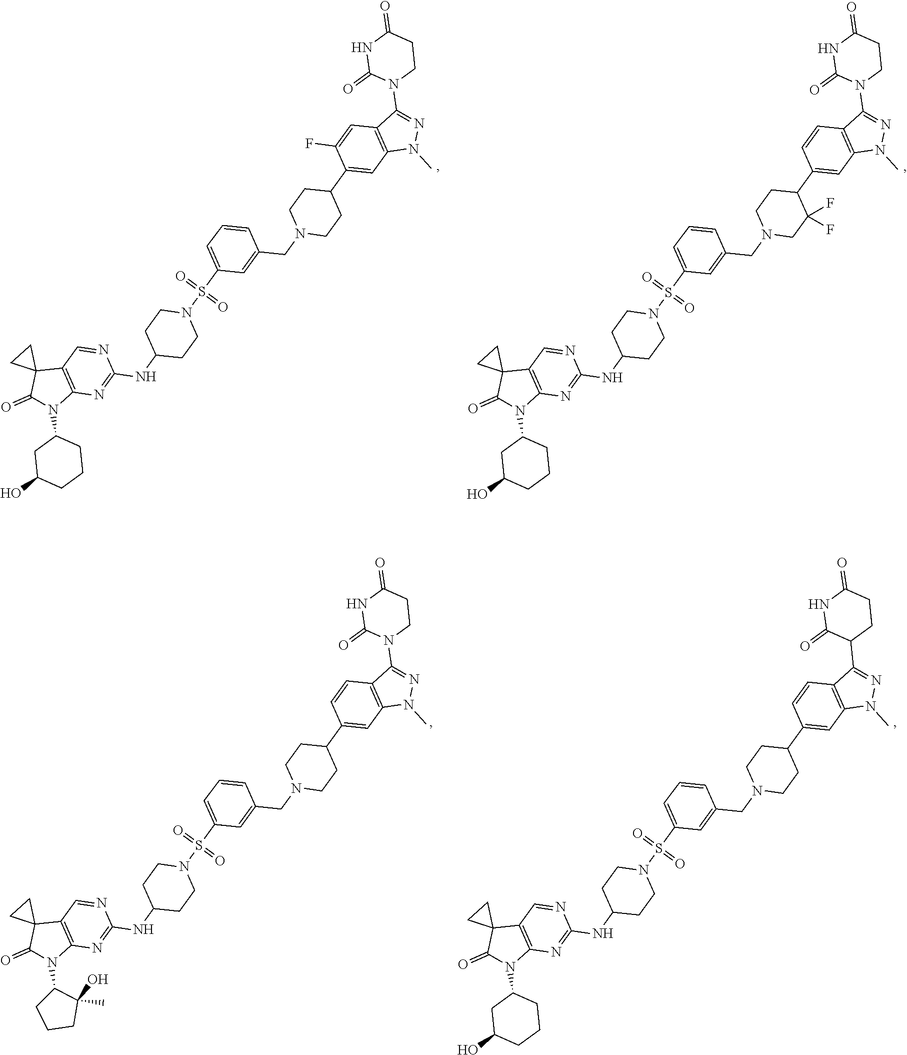 [SMILES: Cn1nc(N2CCC(=O)NC2=O)c2cc(F)c(C3CCN(Cc4cccc(S(=O)(=O)N5CCC(Nc6ncc7c(n6)N([C@@H]6CCC[C@@H](O)C6)C(=O)C76CC6)CC5)c4)CC3)cc21 ; Cn1nc(N2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)c4)CC3(F)F)cc21 ; Cn1nc(N2CCC(=O)NC2=O)c2ccc(C3CCN(Cc4cccc(S(=O)(=O)N5CCC(Nc6ncc7c(n6)N([C@H]6CCC[C@]6(C)O)C(=O)C76CC6)CC5)c4)CC3)cc21 ; Cn1nc(C2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)c4)CC3)cc21]

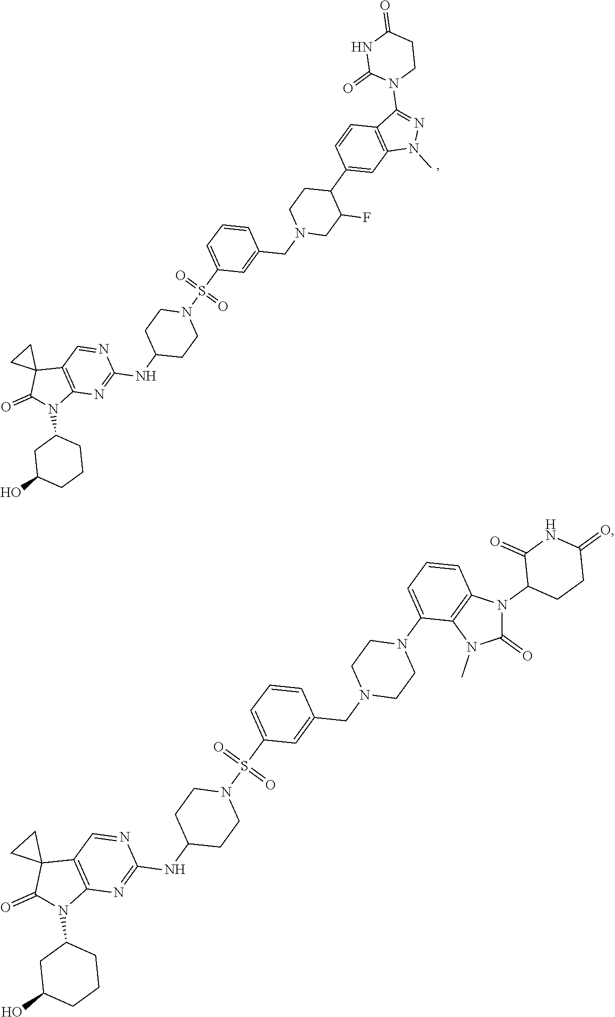 [SMILES: Cn1nc(N2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)c4)CC3F)cc21 ; Cn1c(=O)n(C2CCC(=O)NC2=O)c2cccc(N3CCN(Cc4cccc(S(=O)(=O)N5CCC(Nc6ncc7c(n6)N([C@@H]6CCC[C@@H](O)C6)C(=O)C76CC6)CC5)c4)CC3)c21]

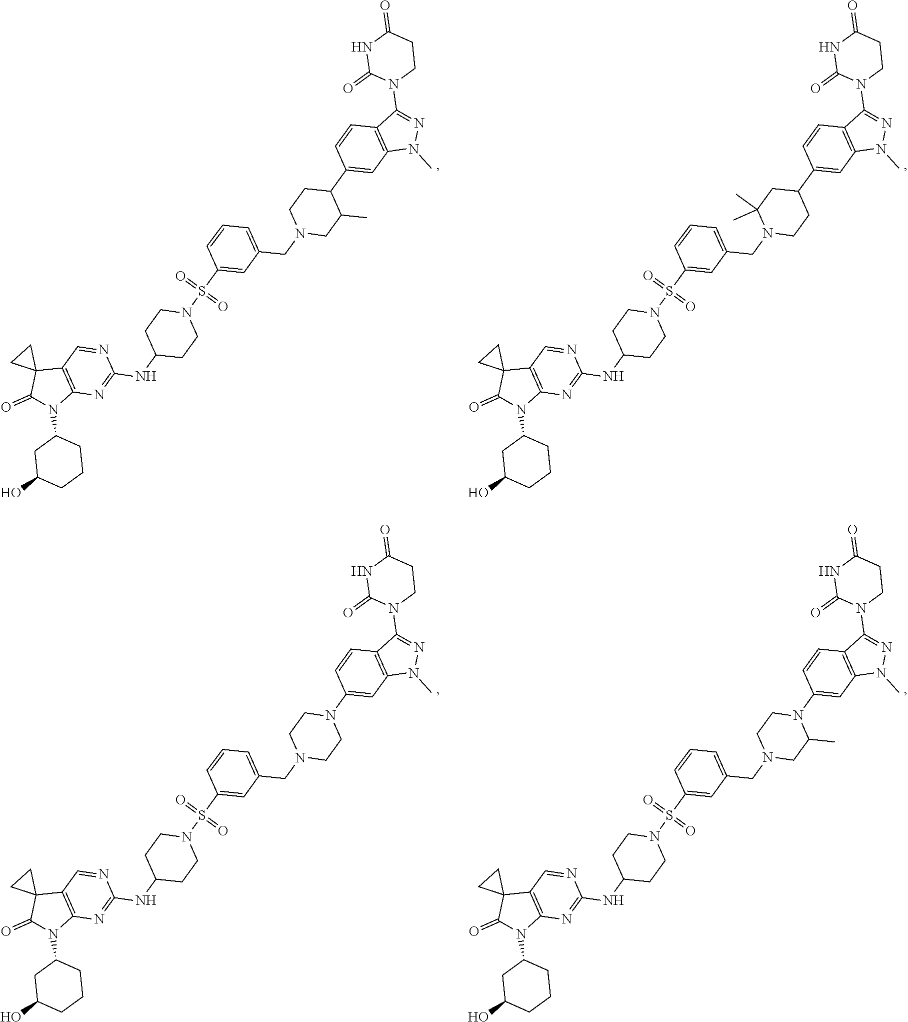 [SMILES: CC1CN(Cc2cccc(S(=O)(=O)N3CCC(Nc4ncc5c(n4)N([C@@H]4CCC[C@@H](O)C4)C(=O)C54CC4)CC3)c2)CCC1c1ccc2c(N3CCC(=O)NC3=O)nn(C)c2c1 ; Cn1nc(N2CCC(=O)NC2=O)c2ccc(C3CCN(Cc4cccc(S(=O)(=O)N5CCC(Nc6ncc7c(n6)N([C@@H]6CCC[C@@H](O)C6)C(=O)C76CC6)CC5)c4)C(C)(C)C3)cc21 ; Cn1nc(N2CCC(=O)NC2=O)c2ccc(N3CCN(Cc4cccc(S(=O)(=O)N5CCC(Nc6ncc7c(n6)N([C@@H]6CCC[C@@H](O)C6)C(=O)C76CC6)CC5)c4)CC3)cc21 ; CC1CN(Cc2cccc(S(=O)(=O)N3CCC(Nc4ncc5c(n4)N([C@@H]4CCC[C@@H](O)C4)C(=O)C54CC4)CC3)c2)CCN1c1ccc2c(N3CCC(=O)NC3=O)nn(C)c2c1]

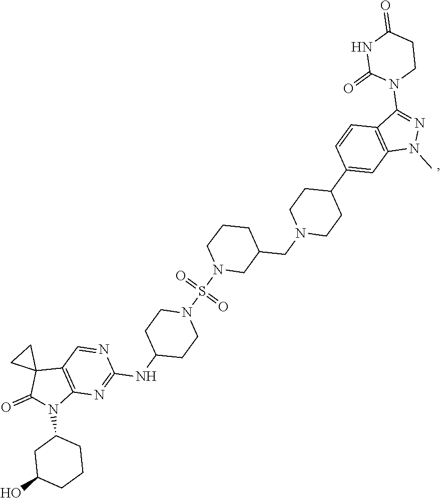 [SMILES: Cn1nc(N2CCC(=O)NC2=O)c2ccc(C3CCN(CC4CCCN(S(=O)(=O)N5CCC(Nc6ncc7c(n6)N([C@@H]6CCC[C@@H](O)C6)C(=O)C76CC6)CC5)C4)CC3)cc21]

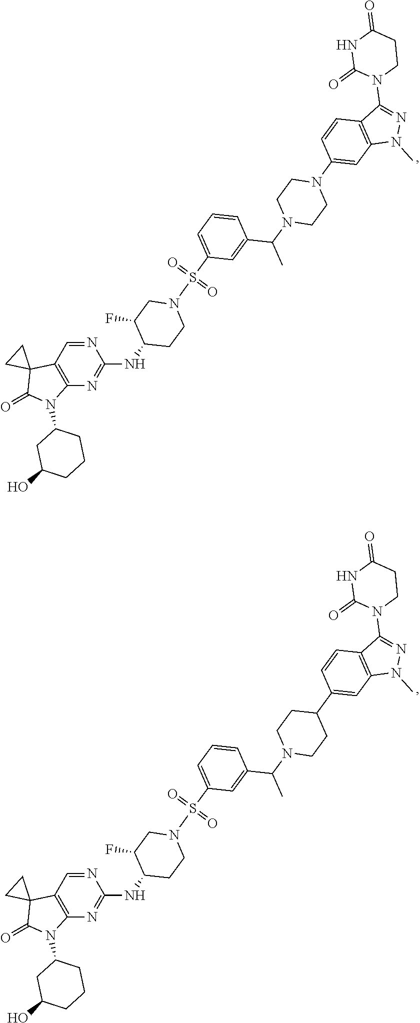 [SMILES: CC(c1cccc(S(=O)(=O)N2CC[C@H](Nc3ncc4c(n3)N([C@@H]3CCC[C@@H](O)C3)C(=O)C43CC3)[C@H](F)C2)c1)N1CCN(c2ccc3c(N4CCC(=O)NC4=O)nn(C)c3c2)CC1 ; CC(c1cccc(S(=O)(=O)N2CC[C@H](Nc3ncc4c(n3)N([C@@H]3CCC[C@@H](O)C3)C(=O)C43CC3)[C@H](F)C2)c1)N1CCC(c2ccc3c(N4CCC(=O)NC4=O)nn(C)c3c2)CC1]

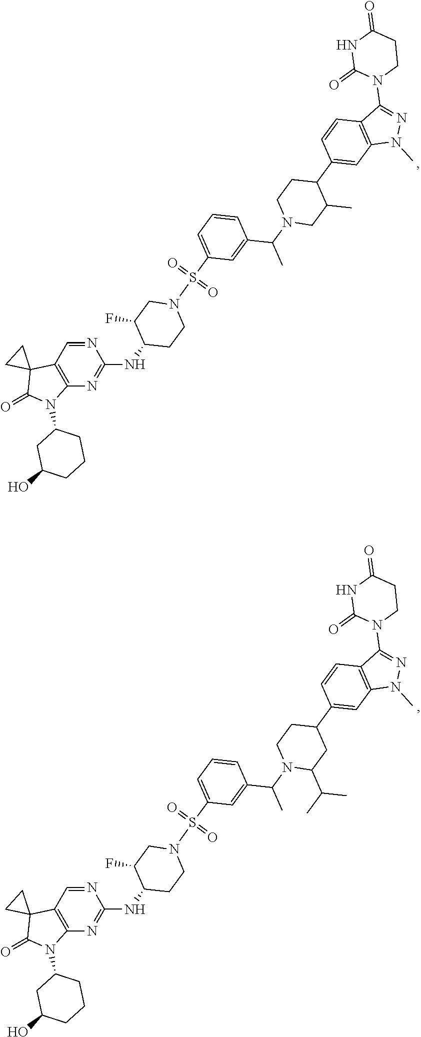 [SMILES: CC1CN(C(C)c2cccc(S(=O)(=O)N3CC[C@H](Nc4ncc5c(n4)N([C@@H]4CCC[C@@H](O)C4)C(=O)C54CC4)[C@H](F)C3)c2)CCC1c1ccc2c(N3CCC(=O)NC3=O)nn(C)c2c1 ; CC(C)C1CC(c2ccc3c(N4CCC(=O)NC4=O)nn(C)c3c2)CCN1C(C)c1cccc(S(=O)(=O)N2CC[C@H](Nc3ncc4c(n3)N([C@@H]3CCC[C@@H](O)C3)C(=O)C43CC3)[C@H](F)C2)c1]

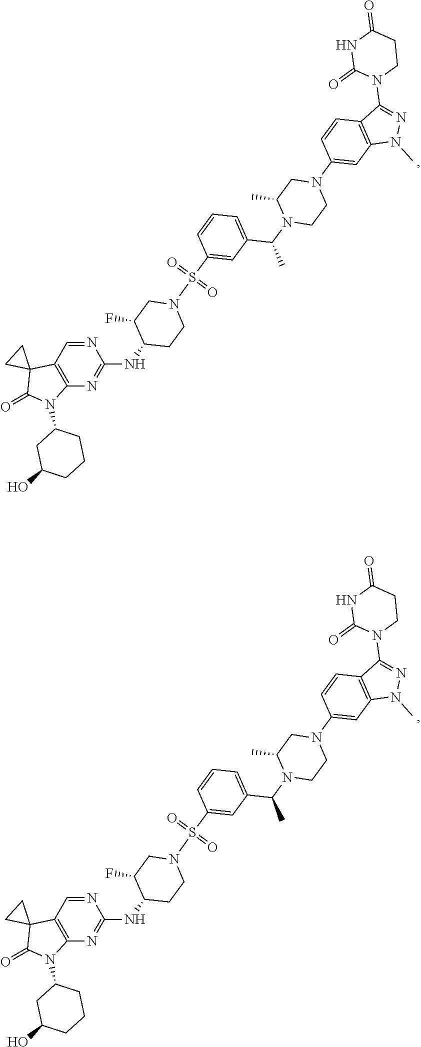 [SMILES: C[C@@H]1CN(c2ccc3c(N4CCC(=O)NC4=O)nn(C)c3c2)CCN1[C@H](C)c1cccc(S(=O)(=O)N2CC[C@H](Nc3ncc4c(n3)N([C@@H]3CCC[C@@H](O)C3)C(=O)C43CC3)[C@H](F)C2)c1 ; C[C@@H]1CN(c2ccc3c(N4CCC(=O)NC4=O)nn(C)c3c2)CCN1[C@@H](C)c1cccc(S(=O)(=O)N2CC[C@H](Nc3ncc4c(n3)N([C@@H]3CCC[C@@H](O)C3)C(=O)C43CC3)[C@H](F)C2)c1]

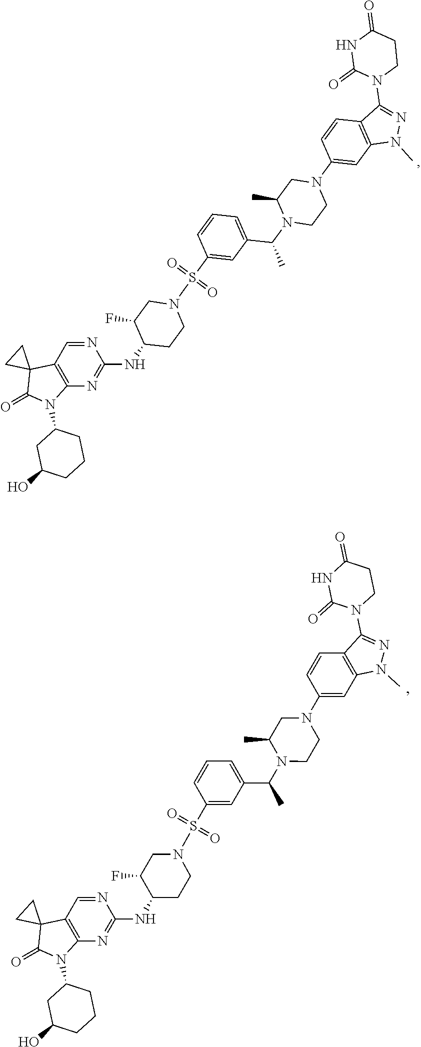 [SMILES: C[C@H](c1cccc(S(=O)(=O)N2CC[C@H](Nc3ncc4c(n3)N([C@@H]3CCC[C@@H](O)C3)C(=O)C43CC3)[C@H](F)C2)c1)N1CCN(c2ccc3c(N4CCC(=O)NC4=O)nn(C)c3c2)C[C@@H]1C ; C[C@H]1CN(c2ccc3c(N4CCC(=O)NC4=O)nn(C)c3c2)CCN1[C@@H](C)c1cccc(S(=O)(=O)N2CC[C@H](Nc3ncc4c(n3)N([C@@H]3CCC[C@@H](O)C3)C(=O)C43CC3)[C@H](F)C2)c1]

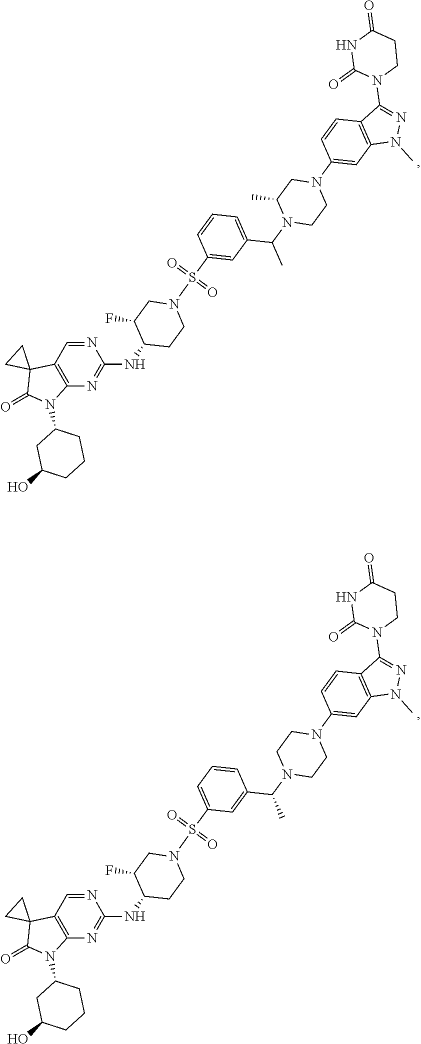 [SMILES: CC(c1cccc(S(=O)(=O)N2CC[C@H](Nc3ncc4c(n3)N([C@@H]3CCC[C@@H](O)C3)C(=O)C43CC3)[C@H](F)C2)c1)N1CCN(c2ccc3c(N4CCC(=O)NC4=O)nn(C)c3c2)C[C@H]1C ; C[C@H](c1cccc(S(=O)(=O)N2CC[C@H](Nc3ncc4c(n3)N([C@@H]3CCC[C@@H](O)C3)C(=O)C43CC3)[C@H](F)C2)c1)N1CCN(c2ccc3c(N4CCC(=O)NC4=O)nn(C)c3c2)CC1]

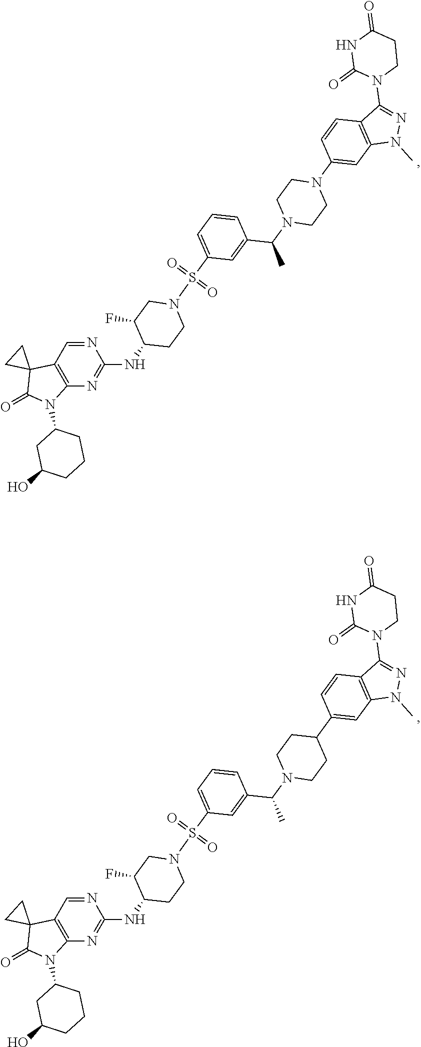 [SMILES: C[C@@H](c1cccc(S(=O)(=O)N2CC[C@H](Nc3ncc4c(n3)N([C@@H]3CCC[C@@H](O)C3)C(=O)C43CC3)[C@H](F)C2)c1)N1CCN(c2ccc3c(N4CCC(=O)NC4=O)nn(C)c3c2)CC1 ; C[C@H](c1cccc(S(=O)(=O)N2CC[C@H](Nc3ncc4c(n3)N([C@@H]3CCC[C@@H](O)C3)C(=O)C43CC3)[C@H](F)C2)c1)N1CCC(c2ccc3c(N4CCC(=O)NC4=O)nn(C)c3c2)CC1]

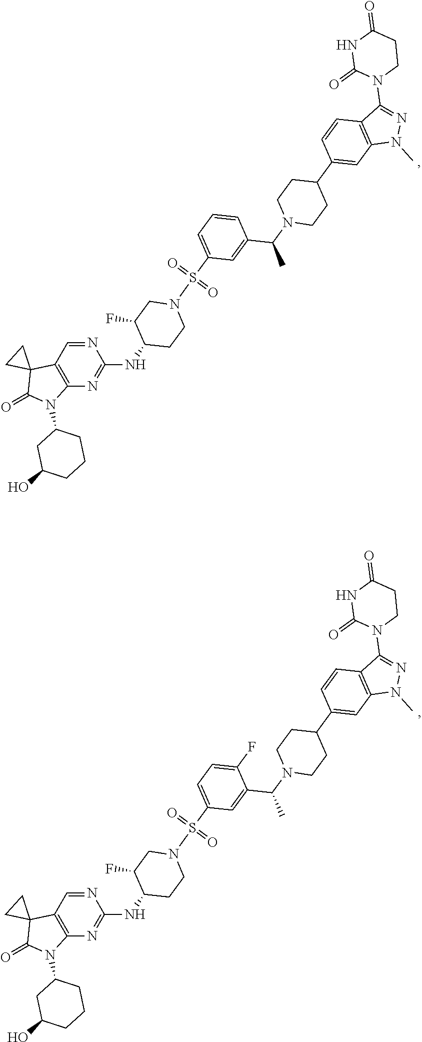 [SMILES: C[C@@H](c1cccc(S(=O)(=O)N2CC[C@H](Nc3ncc4c(n3)N([C@@H]3CCC[C@@H](O)C3)C(=O)C43CC3)[C@H](F)C2)c1)N1CCC(c2ccc3c(N4CCC(=O)NC4=O)nn(C)c3c2)CC1 ; C[C@H](c1cc(S(=O)(=O)N2CC[C@H](Nc3ncc4c(n3)N([C@@H]3CCC[C@@H](O)C3)C(=O)C43CC3)[C@H](F)C2)ccc1F)N1CCC(c2ccc3c(N4CCC(=O)NC4=O)nn(C)c3c2)CC1]

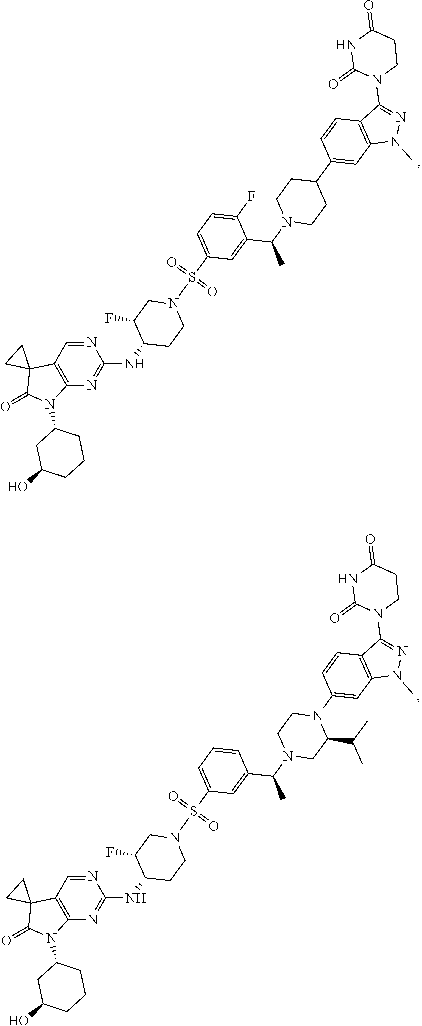 [SMILES: C[C@@H](c1cc(S(=O)(=O)N2CC[C@H](Nc3ncc4c(n3)N([C@@H]3CCC[C@@H](O)C3)C(=O)C43CC3)[C@H](F)C2)ccc1F)N1CCC(c2ccc3c(N4CCC(=O)NC4=O)nn(C)c3c2)CC1 ; CC(C)[C@H]1CN([C@@H](C)c2cccc(S(=O)(=O)N3CC[C@H](Nc4ncc5c(n4)N([C@@H]4CCC[C@@H](O)C4)C(=O)C54CC4)[C@H](F)C3)c2)CCN1c1ccc2c(N3CCC(=O)NC3=O)nn(C)c2c1]

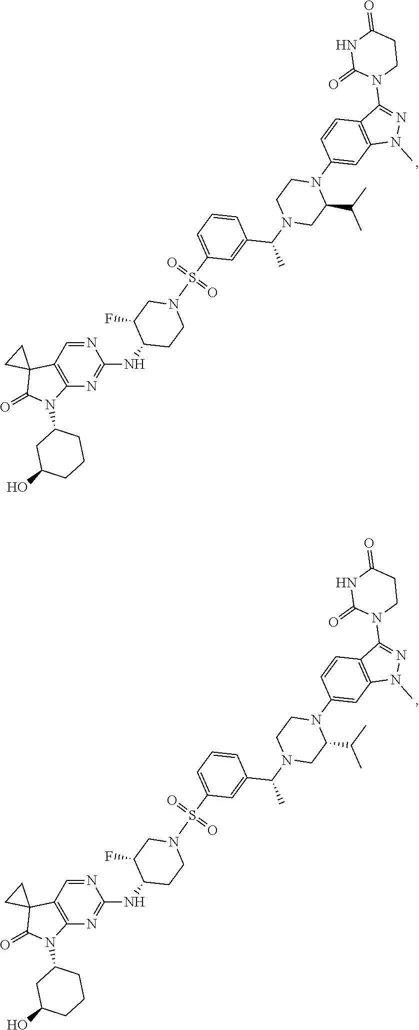 [SMILES: CC(C)[C@H]1CN([C@H](C)c2cccc(S(=O)(=O)N3CC[C@H](Nc4ncc5c(n4)N([C@@H]4CCC[C@@H](O)C4)C(=O)C54CC4)[C@H](F)C3)c2)CCN1c1ccc2c(N3CCC(=O)NC3=O)nn(C)c2c1 ; CC(C)[C@@H]1CN([C@H](C)c2cccc(S(=O)(=O)N3CC[C@H](Nc4ncc5c(n4)N([C@@H]4CCC[C@@H](O)C4)C(=O)C54CC4)[C@H](F)C3)c2)CCN1c1ccc2c(N3CCC(=O)NC3=O)nn(C)c2c1]

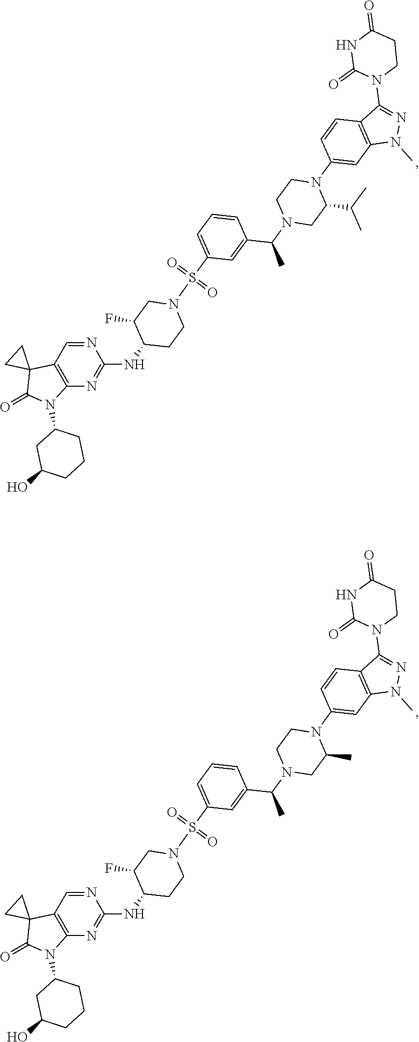 [SMILES: CC(C)[C@@H]1CN([C@@H](C)c2cccc(S(=O)(=O)N3CC[C@H](Nc4ncc5c(n4)N([C@@H]4CCC[C@@H](O)C4)C(=O)C54CC4)[C@H](F)C3)c2)CCN1c1ccc2c(N3CCC(=O)NC3=O)nn(C)c2c1 ; C[C@@H](c1cccc(S(=O)(=O)N2CC[C@H](Nc3ncc4c(n3)N([C@@H]3CCC[C@@H](O)C3)C(=O)C43CC3)[C@H](F)C2)c1)N1CCN(c2ccc3c(N4CCC(=O)NC4=O)nn(C)c3c2)[C@@H](C)C1]

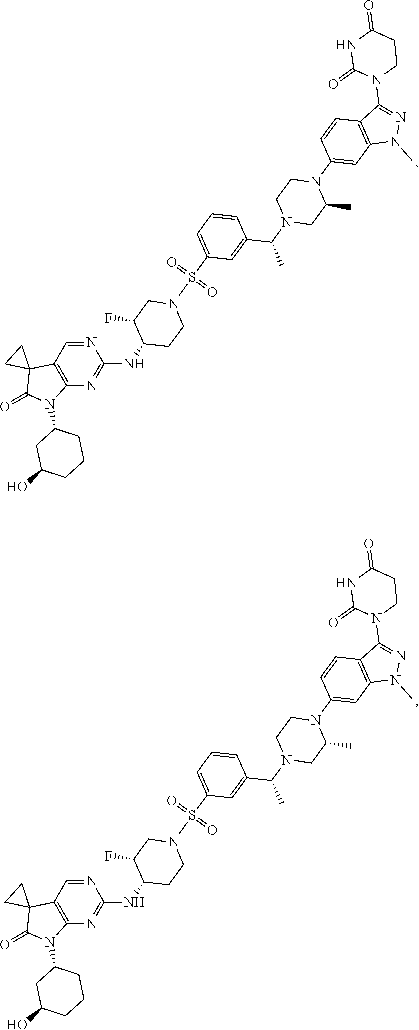 [SMILES: C[C@H](c1cccc(S(=O)(=O)N2CC[C@H](Nc3ncc4c(n3)N([C@@H]3CCC[C@@H](O)C3)C(=O)C43CC3)[C@H](F)C2)c1)N1CCN(c2ccc3c(N4CCC(=O)NC4=O)nn(C)c3c2)[C@@H](C)C1 ; C[C@H](c1cccc(S(=O)(=O)N2CC[C@H](Nc3ncc4c(n3)N([C@@H]3CCC[C@@H](O)C3)C(=O)C43CC3)[C@H](F)C2)c1)N1CCN(c2ccc3c(N4CCC(=O)NC4=O)nn(C)c3c2)[C@H](C)C1]

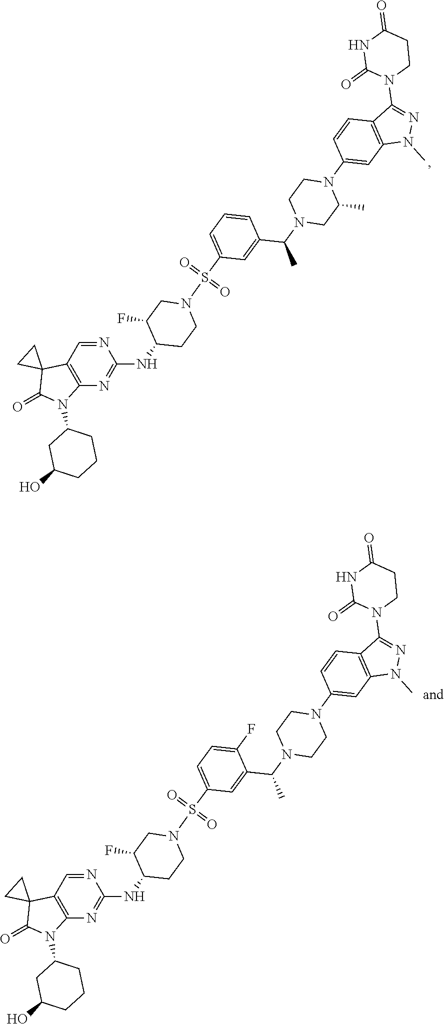 [SMILES: C[C@@H]1CN([C@@H](C)c2cccc(S(=O)(=O)N3CC[C@H](Nc4ncc5c(n4)N([C@@H]4CCC[C@@H](O)C4)C(=O)C54CC4)[C@H](F)C3)c2)CCN1c1ccc2c(N3CCC(=O)NC3=O)nn(C)c2c1 ; C[C@H](c1cc(S(=O)(=O)N2CC[C@H](Nc3ncc4c(n3)N([C@@H]3CCC[C@@H](O)C3)C(=O)C43CC3)[C@H](F)C2)ccc1F)N1CCN(c2ccc3c(N4CCC(=O)NC4=O)nn(C)c3c2)CC1]

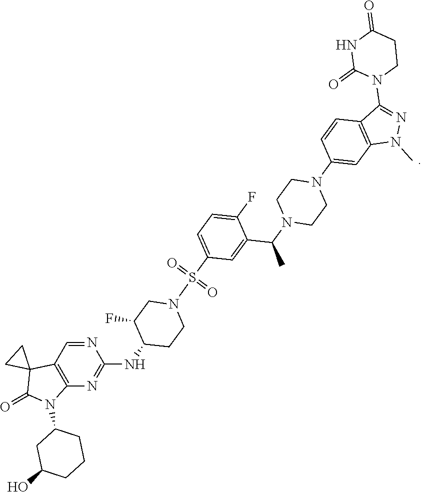 [SMILES: C[C@@H](c1cc(S(=O)(=O)N2CC[C@H](Nc3ncc4c(n3)N([C@@H]3CCC[C@@H](O)C3)C(=O)C43CC3)[C@H](F)C2)ccc1F)N1CCN(c2ccc3c(N4CCC(=O)NC4=O)nn(C)c3c2)CC1]

76. (canceled)

77. A method of inhibiting CDK2 and/or CCNE (CCNE1 and/or CCNE2) signaling in a sample by contacting CDK2 and/or CCNE (CCNE1 and/or CCNE2) with a compound of claim 1, or a pharmaceutically acceptable salt thereof.

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)
